(19) United States
(12) Patent Application Publication
(10) Pub. No.: US 2026/0274814 A1
(43) Pub. Date: Sep. 17, 2026

(54) THIENYL COMPOUNDS FOR THE TREATMENT OF A CENTRAL NERVOUS SYSTEM DISEASE OR DISORDER

(71) Applicant: Coreterra Therapeutics, Inc., Rochelle Park, NJ (US)

(72) Inventors: Daniel Wintner GOODMAN, Old Greenwich, CT (US); Leslie Joseph STREET, Laguna Niguel, CA (US); Gang ZHOU, Bridgewater, NJ (US); Lee Apostle MCDERMOTT, Pittsburgh, PA (US); Larry Wendell HARDY, North Grafton, MA (US)

(21) Appl. No.: 19/665,285

(22) Filed: May 1, 2026

Related U.S. Application Data
(63) Continuation of application No. PCT/US2024/054334, filed on Nov. 4, 2024.
(60) Provisional application No. 63/595,716, filed on Nov. 2, 2023.

(51) Int. Cl. C07D 333/54; A61K 31/381; A61K 31/4025; A61K 31/5377; C07D 333/58; C07D 333/62; C07D 333/68; C07D 333/72; C07D 333/78; C07D 333/80; C07D 409/12; C07D 495/04

(52) U.S. Cl. CPC C07D 333/54; A61K 31/381; A61K 31/4025; A61K 31/5377; C07D 333/58; C07D 333/62; C07D 333/68; C07D 333/72; C07D 333/78; C07D 333/80; C07D 409/12; C07D 495/04

(57) ABSTRACT

Disclosed herein are thienyl compounds and salts, pharmaceutical compositions, and uses thereof.

This application is a bypass continuation of International Application No. PCT/US2024/054334, filed Nov. 4, 2024, which claims the benefit of priority of U.S. provisional application No. 63/595,716, filed Nov. 2, 2023, the contents of which are incorporated by reference as if written herein in their entireties.

Central nervous system (CNS) diseases and disorders affect a wide range of the population with differing severity. These diseases and disorders can affect a person's thoughts, mood, behavior, and social interactions and can significantly impair daily functioning. See, e.g., Diagnostic and Statistical Manual of Mental Disorders, 4th Ed., American Psychiatric Association (2000) (“DSM-IV-TR”); Diagnostic and Statistical Manual of Mental Disorders, 5th Ed., American Psychiatric Association (2013) (“DSM-5”).

There remains a need for effective treatments for CNS diseases and disorders, including depression, schizophrenia, and anxiety disorders. The present disclosure provides treatments to meet these critical needs.

### SUMMARY

Provided is a compound of Formula I:

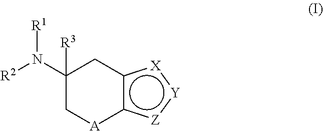

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H, methyl, and ethyl, and R² is chosen from H, C₁-C₃ alkyl optionally substituted by one C₃-C₅ saturated cycloalkyl, 3-5 membered saturated heterocycloalkyl, and C₃-C₅ saturated cycloalkyl optionally substituted by one or more halogen, or R¹ and R², taken together with the intervening nitrogen atom, form a 4-6 membered saturated heterocyclic ring;
   R³ is H or C₁-C₃ alkyl;
   R⁴, R⁵, and R⁶ are independently chosen from H, C₁-C₄ alkyl optionally substituted by one NR¹⁰R¹¹, C₁-C₄ haloalkyl, halogen, CN, phenyl, and C₃-C₅ saturated cycloalkyl; and
   R¹⁰ and R¹¹ are independently chosen from H and C₁-C₄ alkyl;
provided that:
   a) R² is H only if R¹ is H;
   b) if A is —CH₂—, X is S, and R¹, R², R³ and R⁵ are H, then R⁶ is not H, methyl, CN, cyclopentyl, isobutyl, t-butyl, phenyl, CF₃, or CH₂CH₂N(CH₃)₂;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
   f) the compound of Formula I is not

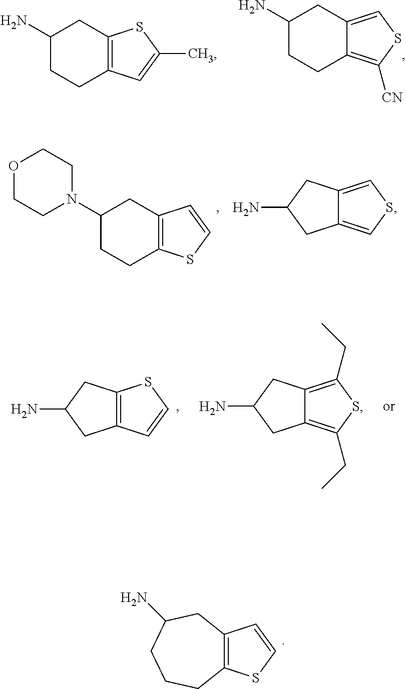

Also provided is a compound or pharmaceutically acceptable salt thereof described herein that is at least 90% enantiomerically pure.

Also provided is a pharmaceutical composition comprising a compound or pharmaceutically acceptable salt described herein together with at least one pharmaceutically acceptable excipient.

Also provided is a method of treatment of a central nervous system disease or disorder comprising the administration of a therapeutically effective amount of a compound or pharmaceutically acceptable salt described herein, or a pharmaceutical composition described herein, to a patient in need thereof, wherein the central nervous system disease or disorder is depression, schizophrenia, or an anxiety disorder.

These and other aspects of the disclosure will be apparent upon reference to the following description. To this end, various references are set forth herein which describe in more detail certain background information, procedures, compounds, and/or compositions, and are each hereby incorporated by reference in their entirety.

### DESCRIPTION

This specification is intended only to acquaint others skilled in the art with the present disclosure, its principles, and its practical application so that others skilled in the art may adapt and apply the disclosure in its numerous forms and is intended for purposes of illustration only. The scope of the invention is not limited to the specific embodiments or examples described.

### Definitions

The term “alkoxy” refers to an alkyl ether radical attached to a parent molecular moiety via the oxygen atom. Examples of suitable alkyl ether radicals include methoxy, ethoxy, n-propoxy, isopropoxy, n-butoxy, iso-butoxy, sec-butoxy, tert-butoxy, and the like.

The term “alkyl” refers to a straight-chain or branched-chain saturated hydrocarbon radical containing from 1 to 20 carbon atoms. In certain embodiments, said alkyl will comprise from 1 to 10 carbon atoms. In further embodiments, said alkyl will comprise from 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, nonyl and the like. An alkyl group can be substituted by other atom(s) or group(s), but unless otherwise specified herein is unsubstituted.

The term “alkylene” refers to a saturated aliphatic group derived from a straight or branched chain saturated hydrocarbon attached at two or more positions, such as methylene (—CH₂—). Unless otherwise specified, the term “alkyl” may include “alkylene” groups. An alkylene group can be substituted by other atom(s) or group(s), but unless otherwise specified herein is unsubstituted.

The term “alkylamino” refers to an alkyl group attached to a parent molecular moiety via a nitrogen atom. Suitable alkylamino groups may be mono- or dialkylated, forming groups such as, for example, N-methylamino, N-ethylamino, N,N-dimethylamino, N,N-ethylmethylamino and the like.

The term “aryl” refers to a monocyclic, fused bicyclic, or fused tricyclic ring system in which all of the ring atoms are carbon atoms and at least one of the rings is aromatic. The term “aryl” embraces aromatic groups such as phenyl, naphthyl, anthracenyl, and phenanthryl. An aryl group can be substituted by other atom(s) or group(s), but unless otherwise specified herein is unsubstituted.

The term “carboxyl” or “carboxy” refers to —C(O)OH or the corresponding “carboxylate” anion, such as is in a carboxylic acid salt.

The term “cyano” refers to —CN.

The term “cycloalkyl” or “carbocycle” refers to a non-aromatic saturated or partially unsaturated (if not defined as “saturated”) monocyclic, or fused, bridged, and/or spiro bicyclic or tricyclic, group in which all of the ring atoms are carbon atoms. In certain embodiments, said cycloalkyl will comprise from 3 to 7 carbon atoms. Examples of such cycloalkyl groups include cyclopropyl, cyclobutyl, cyclopentyl, cyclohexyl, cycloheptyl and the like. Examples also include decahydronaphthalene, octahydronaphthalene, bicyclo[1.1.1]pentane, adamantane, and bicyclo[3.2.1]octane. A cycloalkyl group can be substituted by other atom(s) or group(s), but unless otherwise specified herein is unsubstituted.

The term “halo” or “halogen” refers to fluorine, chlorine, bromine, or iodine.

The term “haloalkoxy” refers to a haloalkyl group attached to a parent molecular moiety via an oxygen atom.

The term “haloalkyl” refers to an alkyl radical wherein one or more hydrogens is replaced with a halogen. Specifically embraced are monohaloalkyl, dihaloalkyl, trihaloalkyl, and polyhaloalkyl radicals. A monohaloalkyl radical, for one example, may have an iodo, bromo, chloro or fluoro atom within the radical. Dihalo, trihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” or “heteroaryl group” refers to (a) a 5 or 6 membered monocyclic aromatic ring in which the ring contains at least one heteroatom, such as nitrogen, oxygen or sulfur, and (b) 7-15 membered bicyclic and tricyclic rings in which at least one of the rings is aromatic, and at least one of the rings contains at least one heteroatom, such as nitrogen, oxygen or sulfur. Heteroaryl groups may be bridged, spiro, and/or fused. Examples include, but are not limited to, 2,3-dihydrobenzofuranyl, 1,2-dihydroquinolinyl, 3,4-dihydroisoquinolinyl, 1,2,3,4-tetrahydroisoquinolinyl, 1,2,3,4-tetrahydroquinolinyl, benzoxazinyl, benzthiazinyl, chromanyl, furanyl, 2-furanyl, 3-furanyl, imidazolyl, isoxazolyl, isothiazolyl, oxadiazolyl, oxazolyl, pyridinyl, 2-, 3-, or 4-pyridinyl, pyrimidinyl, 2-, 4-, or 5-pyrimidinyl, pyrazolyl, pyrrolyl, 2- or 3-pyrrolyl, pyrazinyl, pyridazinyl, 3- or 4-pyridazinyl, 2-pyrazinyl, thienyl, 2-thienyl, 3-thienyl, tetrazolyl, thiazolyl, thiadiazolyl, triazinyl, triazolyl, pyridin-2-yl, pyridin-4-yl, pyrimidin-2-yl, pyridazin-4-yl, pyrazin-2-yl, naphthyridinyl, pteridinyl, phthalazinyl, purinyl, alloxazinyl, benzimidazolyl, benzofuranyl, benzofurazanyl, 2H-1-benzopyranyl, benzothiadiazine, benzothiazinyl, benzothiazolyl, benzothiophenyl, benzoxazolyl, cinnolinyl, furopyridinyl, indolinyl, indolizinyl, indolyl, or 2-, 3-, 4-, 5-, 6-, or 7-indolyl, 3H-indolyl, quinazolinyl, quinoxalinyl, isoindolyl, isoquinolinyl, and 2,3,4,5-tetrahydro-benzo[f][1,4]oxazepinyl. A heteroaryl group may contain 5, 6, or 8-15 ring atoms. A heteroaryl group may contain 5 to 10 ring atoms, such as 5, 6, 9, or 10 ring atoms. A heteroaryl group can be substituted by other atom(s) or group(s), but unless otherwise specified herein is unsubstituted.

The term “heterocycloalkyl” or “heterocycloalkyl group” or “heterocycle” or “heterocyclic” or “heterocyclyl” refers to 3-15 membered monocyclic, bicyclic, and tricyclic non-aromatic, saturated or partially unsaturated (if not defined as “saturated”) rings containing at least one heteroatom, such as nitrogen, oxygen, sulfur or phosphorus. Heterocycloalkyl groups may be bridged, spiro, and/or fused. Examples include, but are not limited to, tetrahydrofuranyl, pyrrolidinyl, pyrrolinyl, imidazolidinyl, imidazolinyl, pyrazolidinyl, pyrazolinyl, piperidyl, piperazinyl, indolinyl, isoindolinyl, morpholinyl, thiomorpholinyl, homomorpholinyl, homopiperidyl, homopiperazinyl, pyrrolidinyl, tetrahydropyranyl, piperidinyl, tetrahydrothienyl, homopiperidinyl, oxazolidinonyl, dihydropyrazolyl, dihydropyrrolyl, dihydropyrazinyl, dihydropyridinyl, dihydropyrimidinyl, dihydrofuryl, dihydropyranyl, 3,8-diaza-bicyclo[3.2.1]octanyl, 2,5-diaza-bicyclo[2.2.1]heptanyl, 3,8-diaza-bicyclo[3.2.1]octanyl, 3,9-diaza-bicyclo[4.2.1]nonanyl, and 2,6-diaza-bicyclo[3.2.2]nonanyl. A heterocycloalkyl group may contain, in addition to carbon ring atom(s), at least one nitrogen, oxygen, or sulfur ring atom. For example, a heterocycloalkyl group may contain, in addition to carbon ring atom(s), at least one nitrogen or oxygen ring atom. A heterocycloalkyl group may contain, in addition to carbon ring atom(s), at least one nitrogen ring atom. A heterocycloalkyl group may contain carbon ring atoms and 1 to 5 nitrogen, oxygen, and/or sulfur ring atoms. A heterocycloalkyl group may contain carbon ring atoms and at least one oxygen ring atom. A heterocycloalkyl group may contain carbon ring atoms and 1 to 3 nitrogen, oxygen, and/or sulfur ring atoms. A heterocycloalkyl group may contain carbon ring atoms and 1 to 3 nitrogen and oxygen ring atoms. A heterocycloalkyl group may contain carbon ring atoms and 1 to 4 nitrogen, oxygen, and/or sulfur ring atoms. A heterocycloalkyl group may contain from 3 to 10 ring atoms. A heterocycloalkyl group may contain from 3 to 7 ring atoms. A heterocycloalkyl group may contain from 5 to 7 ring atoms, such as 5 ring atoms, 6 ring atoms, or 7 ring atoms. Unless otherwise indicated, the foregoing heterocycloalkyl groups can be C-attached or N-attached where such is possible and results in the creation of a stable structure. For example, piperidinyl can be piperidin-1-yl (N-attached) or piperidin-4-yl (C-attached). A heterocycloalkyl group can be substituted by other atom(s) or group(s), but unless otherwise specified herein is unsubstituted.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The term “stable” or “chemically stable” refers to a compound that is sufficiently robust to be isolated to a useful degree of purity from a reaction mixture. The present disclosure is directed solely to stable compounds. When substituent definitions used herein include possibilities that, owing to valency requirements, number of available sites for substitution, or other reasons, would not result in a stable compound, the list is intended to be read in context to exclude those possibilities and to include only the options suitable for a stable compound.

Compounds described herein may exist in particular geometric or stereoisomeric forms. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure.

The term “bond” refers to a covalent linkage between two atoms. As used herein, if a variable is defined as a single bond it means that the two atoms to which the variable is attached are joined by a single bond. As used herein, if two variable positions are defined as together forming a bond it means that the two atoms to which the variables are attached are joined by a bond (if the atoms are already joined by a bond, it means that they are joined by an additional bond, e.g., to form a double bond). A dashed line between two atoms in a drawing of a molecule indicates that an additional bond may be present or absent at that position.

“Therapeutically effective amount” refers to an amount of a drug, alone or in combination with another therapy, that is sufficient to effectively treat a disorder, condition, or disease in a particular patient or patient population.

The phrase “pharmaceutically acceptable” refers to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

The term “pharmaceutical composition” refers to the combination of an active agent with a pharmaceutically acceptable excipient, in a form suitable for administration to a patient.

The term “treating” or “treatment” refers to any lessening, reducing, modulating, ameliorating, improving, stabilizing, inhibiting, slowing progression, or delaying onset of a condition, disease, disorder, or the like, or a symptom thereof. For example, treatment may include diminishment of a troublesome symptom of a disorder or disease. As another example, treatment may include slowing the progression of a disease, or preventing or delaying recurrence or symptoms, such as maintenance treatment to prevent or delay relapse. As another example, treatment may include delaying or preventing development or onset of a disease, disorder, or undesirable symptoms, such as prophylactic treatment of an at-risk patient or population.

The term “patient” is generally synonymous with the term “subject” and refers to all mammals including human beings. In preferred embodiments the patient is a human being.

The compounds disclosed herein can exist as salts. Suitable salts include those formed with both organic and inorganic acids or bases. Such salts will normally be pharmaceutically acceptable. However, salts of non-pharmaceutically acceptable salts may be of utility in the preparation and purification of the compound in question. For a more complete discussion of the preparation and selection of salts, refer to Pharmaceutical Salts: Properties, Selection, and Use (Stahl, P. Heinrich. Wiley-VCHA, Zurich, Switzerland, 2002). Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

Chemical names and/or structural formulas described herein that do not specifically recite the isotope ratios of the constituent atoms are intended to include compounds in which the constituent atoms are present in any ratio of isotopes. For example, unless otherwise specified, carbon atoms may be present in any ratio of ¹²C, ¹³C, and ¹⁴C, and hydrogen atoms may be present in any ratio of ¹H, ²H, and ³H. In some embodiments, the constituent atoms in the compounds of the present disclosure are present in their naturally occurring ratios of isotopes. In some embodiments, one or more of the constituent atoms in a compound of the present disclosure is enriched to contain a non-naturally occurring ratio of isotopes.

### DETAILED DESCRIPTION

Provided is a compound of Formula I:

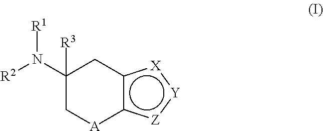

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H, methyl, and ethyl, and R² is chosen from H, C₁-C₃ alkyl optionally substituted by one C₃-C₅ saturated cycloalkyl, 3-5 membered saturated heterocycloalkyl, and C₃-C₅ saturated cycloalkyl optionally substituted by one or more halogen, or R¹ and R², taken together with the intervening nitrogen atom, form a 3-7 membered saturated heterocyclic ring;
   R³ is H or C₁-C₃ alkyl;
   R⁴, R⁵, and R⁶ are independently chosen from H, C₁-C₄ alkyl optionally substituted by one NR¹⁰R¹¹, C₁-C₄ haloalkyl, halogen, CN, phenyl, and C₃-C₅ saturated cycloalkyl; and
   R¹⁰ and R¹¹ are independently chosen from H and C₁-C₄ alkyl;
provided that:
   a) R² is H only if R¹ is H;
   b) if A is —CH₂—, X is S, and R¹, R², R³ and R⁵ are H, then R⁶ is not H, methyl, CN, cyclopentyl, isobutyl, t-butyl, phenyl, CF₃, or CH₂CH₂N(CH₃)₂;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
      f) the compound of Formula I is not

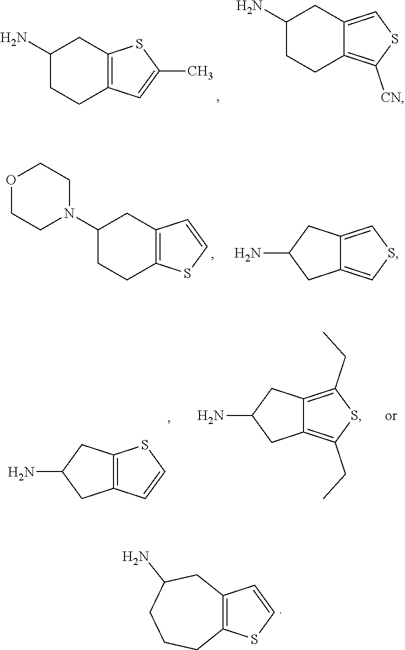

Also provided is a compound of Formula I:

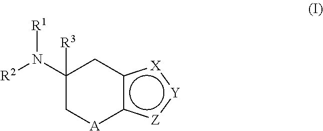

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H, methyl, and ethyl, and R² is chosen from H, C₃-C₅ saturated cycloalkyl, and C₁-C₂ alkyl optionally substituted by one C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₃ alkyl, C₁-C₃ haloalkyl, halogen, CN, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, or CF₃;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
   f) the compound of Formula I is not

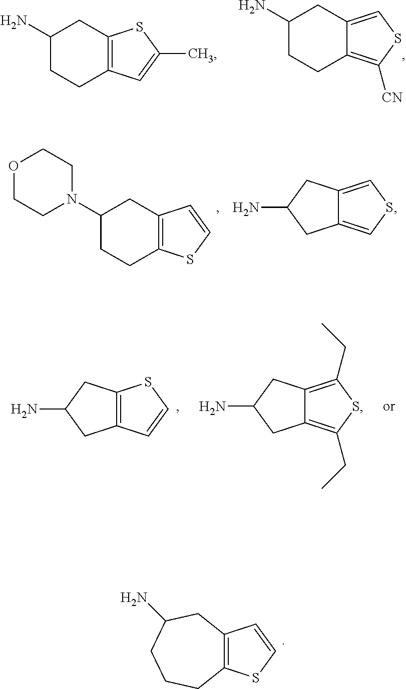

Also provided is a compound of Formula I:

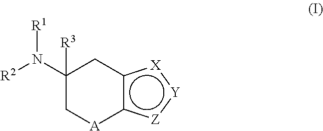

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₃ alkyl, C₁-C₃ haloalkyl, halogen, CN, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, or CF₃;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
   f) the compound of Formula I is not

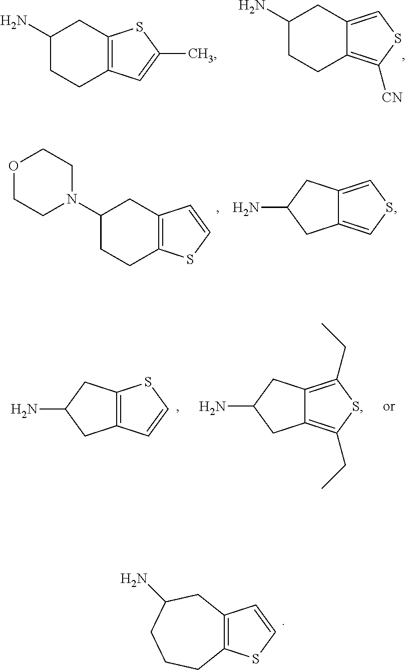

Also provided is a compound of Formula I:

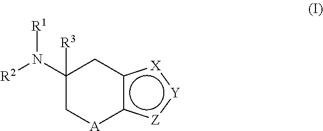

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹, R² and R³ are independently chosen from H and methyl;
   R⁴, R⁵, and R⁶ are independently chosen from H, C₁-C₄ alkyl optionally substituted by one NR¹⁰R¹¹, C₁-C₄ haloalkyl, halogen, CN, phenyl, and C₃-C₅ saturated cycloalkyl; and
   R¹⁰ and R¹¹ are independently chosen from H and C₁-C₄ alkyl;
provided that:
   a) R² is H only if R¹ is H;
   b) if A is —CH₂—, X is S, and R¹, R², R³ and R⁵ are H, then R⁶ is not H, methyl, CN, cyclopentyl, isobutyl, t-butyl, phenyl, CF₃, or CH₂CH₂N(CH₃)₂;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
   f) the compound of Formula I is not

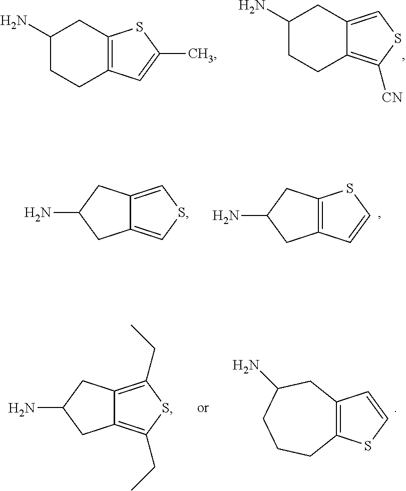

Also provided is a compound of Formula I:

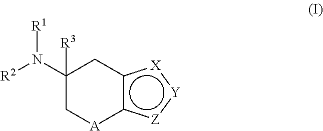

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹ is H or methyl, and R² is chosen from H, cyclopropyl, and methyl optionally substituted by one C₃-C₅ saturated cycloalkyl, or R¹ and R², taken together with the intervening nitrogen atom, form a pyrrolidinyl ring;
   R³ is H or C₁-C₃ alkyl; and
   R⁴, R⁵, and R⁶ are independently chosen from H, C₁-C₃ alkyl, C₁-C₃ haloalkyl, halogen, CN, and cyclopropyl;
provided that:
   a) R² is H only if R¹ is H;
   b) if A is —CH₂—, X is S, and R¹, R², R³ and R⁵ are H, then R⁶ is not H, methyl, CN, or CF₃;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
   f) the compound of Formula I is not

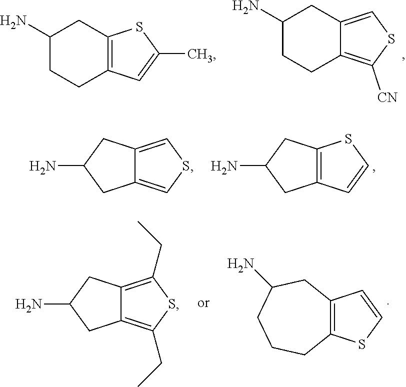

Also provided is a compound of Formula I:

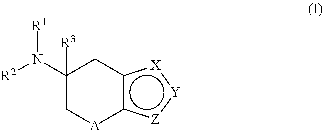

or a pharmaceutically acceptable salt thereof, wherein:
   X is S, Y is CR⁵, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, and cyclopropyl;
provided that:
   a) R² is H only if R¹ is H;
   b) if A is —CH₂—, X is S, and R¹, R², R³ and R⁵ are H, then R⁶ is not H, methyl, CN, isobutyl, t-butyl, or CF₃;
   c) if A is —CH₂—, Z is S, and R¹, R², R³ and R⁴ are H, then R⁵ is not H, CN, Cl, or Br;
   d) if A is —CH₂—, Z is S, and R¹, R³ and R⁴ are H, and R⁵ is H, Cl, or Br, then R² is not methyl; and
   e) the compound of Formula I is not

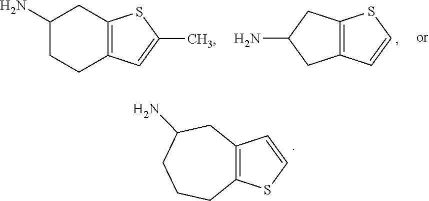

Also provided is a compound of Formula I:

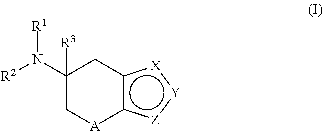

or a pharmaceutically acceptable salt thereof, wherein:
   X is S, Y is CR⁵, and Z is CR⁶; or X is CR⁴, Y is CR⁵, and Z is S;
   A is chosen from a single bond, —CH₂—, —O—, —CH₂—CH₂—, and —CH═CH—;
   R¹ is H or methyl, and R² is H, cyclopropyl, or methyl optionally substituted by one C₃-C₅ saturated cycloalkyl, or R¹ and R², taken together with the intervening nitrogen atom, form a pyrrolidinyl ring;
   R³ is H or C₁-C₃ alkyl; and
   R⁴, R⁵, and R⁶ are independently chosen from H, C₁-C₃ alkyl, C₁-C₃ haloalkyl, halogen, CN, and cyclopropyl;
provided that:
   a) R² is H only if R² is H;
   b) if A is —CH₂—, X is S, and R¹, R², R³ and R⁵ are H, then R⁶ is not H, methyl, CN, or CF₃;
   c) if A is —CH₂—, Z is S, and R¹, R², R³ and R⁴ are H, then R⁵ is not H, CN, Cl, or Br;
   d) if A is —CH₂—, Z is S, and R¹, R³ and R⁴ are H, and R⁵ is H, Cl, or Br, then R² is not methyl; and
   e) the compound of Formula I is not

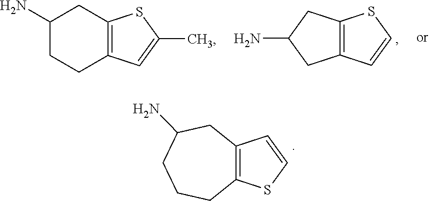

Also provided is a compound of Formula I:

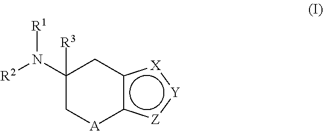

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from —CH₂— and —O—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 3-7 membered saturated heterocyclic ring;
   R³ is H or C₁-C₃ alkyl;
   R⁴, R⁵, and R⁶ are independently chosen from H, C₁-C₄ alkyl optionally substituted by one NR¹⁰R¹¹, C₁-C₄ haloalkyl, halogen, CN, phenyl, and C₃-C₅ saturated cycloalkyl; and
   R¹⁰ and R¹¹ are independently chosen from H and C₁-C₄ alkyl;
provided that:
   a) R² is H only if R¹ is H;
   b) if A is —CH₂—, X is S, and R¹, R², R³ and R⁵ are H, then R⁶ is not H, methyl, CN, cyclopentyl, isobutyl, t-butyl, phenyl, CF₃, or CH₂CH₂N(CH₃)₂;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
      f) the compound of Formula I is not

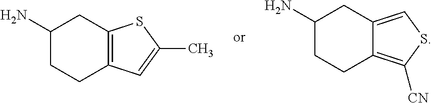

Also provided is a compound of Formula I:

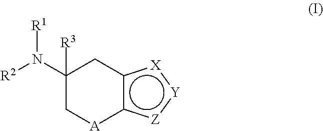

or a pharmaceutically acceptable salt thereof, wherein:
   X is S, Y is CR⁵, and Z is CR⁶; or X is CR⁴, Y is CR⁵, and Z is S;
   A is chosen from —CH₂— and —O—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, phenyl, and C₃-C₅ saturated cycloalkyl; and
provided that:
   a) R² is H only if R¹ is H;
   b) if A is —CH₂—, X is S, and R¹, R², R³ and R⁵ are H, then R⁶ is not H, methyl, CN, cyclopentyl, isobutyl, t-butyl, phenyl, or CF₃;
   c) if A is —CH₂—, Z is S, and R¹, R², R³ and R⁴ are H, then R⁵ is not H, CN, Cl, or Br;
   d) if A is —CH₂—, Z is S, and R¹, R³ and R⁴ are H, and R⁵ is H, Cl, or Br, then R² is not methyl; and
   e) the compound of Formula I is not

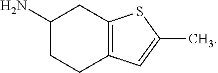 [SMILES: Cc1cc2c(s1)CC(N)CC2]

Also provided is a compound of Formula I:

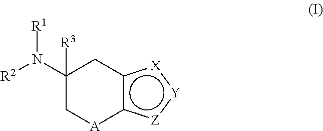

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H, methyl, and ethyl, and R² is chosen from H, C₁-C₃ alkyl optionally substituted by one C₃-C₅ saturated cycloalkyl, 3-5 membered saturated heterocycloalkyl, and C₃-C₅ saturated cycloalkyl optionally substituted by one or more halogen, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₃ alkyl optionally substituted by one NH₂, CF₃, halogen, CN, phenyl, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, phenyl, or CF₃;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
      f) the compound of Formula I is not

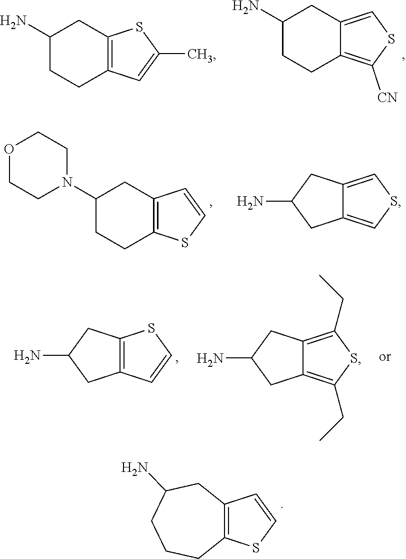

Also provided is a compound of Formula I:

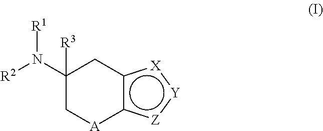

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₃ alkyl optionally substituted by one NH₂, CF₃, halogen, CN, phenyl, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, phenyl, or CF₃;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
   f) the compound of Formula I is not

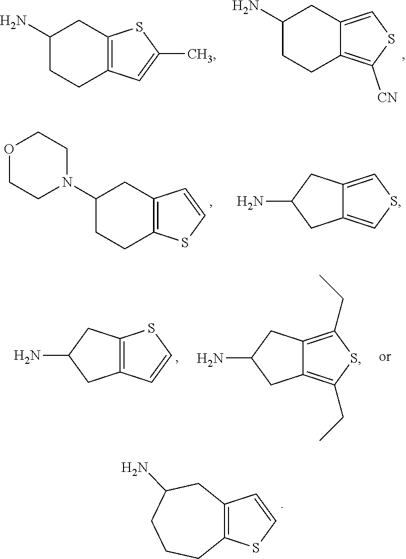

Also provided is a compound of Formula I:

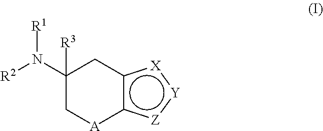

or a pharmaceutically acceptable salt thereof, wherein:
   X is S, Y is CR⁵, and Z is CR⁶; or X is CR⁴, Y is CR⁵, and Z is S;
   A is chosen from a single bond, —CH₂—, —O—, —CH₂—CH₂—, and —CH═CH—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₃ alkyl optionally substituted by one NH₂, CF₃, halogen, CN, phenyl, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, phenyl, or CF₃;
   c) if A is —CH₂—, Z is S, and R¹, R², R³ and R⁴ are H, then R⁵ is not H, CN, Cl, or Br;
   d) if A is —CH₂—, Z is S, and R¹, R³ and R⁴ are H, and R⁵ is H, Cl, or Br, then R² is not methyl; and
      e) the compound of Formula I is not

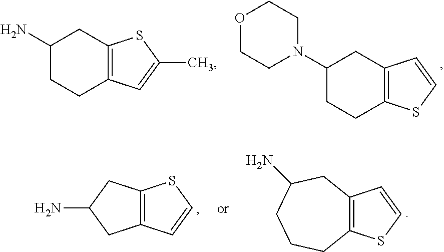 [SMILES: Cc1cc2c(s1)CC(N)CC2 ; c1cc2c(s1)CCC(N1CCOCC1)C2 ; NC1Cc2ccsc2C1 ; NC1CCCc2sccc2C1 ; "."]

Also provided is a compound of Formula I:

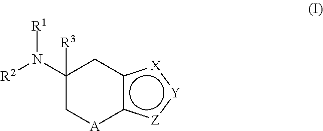

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is chosen from —CH₂— and —O—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₃ alkyl optionally substituted by one NH₂, CF₃, halogen, CN, phenyl, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, phenyl, or CF₃;
   c) if A is —CH₂—, Y is S, and R¹, R², R³ and R⁶ are H, then R⁴ is not H or Br;
   d) if A is —CH₂—, Z is S, and R¹, R², R³ and R⁴ are H, then R⁵ is not H, CN, Cl, or Br;
   e) if A is —CH₂—, Z is S, and R¹, R³ and R⁴ are H, and R⁵ is H, Cl, or Br, then R² is not methyl; and
   f) the compound of Formula I is not

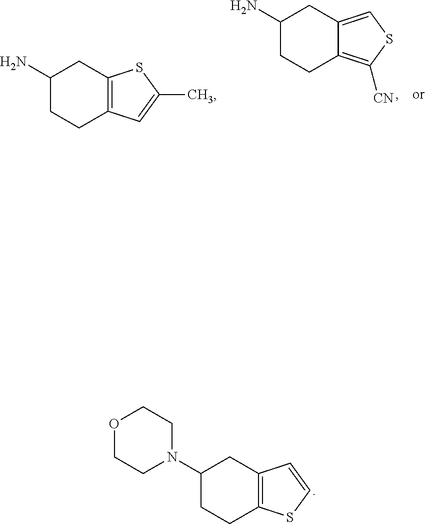 [SMILES: Cc1cc2c(s1)CC(N)CC2 ; N#Cc1scc2c1CCC(N)C2 ; c1cc2c(s1)CCC(N1CCOCC1)C2]

Also provided is a compound of Formula I:

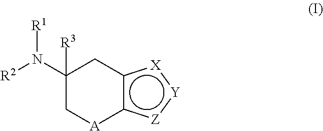

or a pharmaceutically acceptable salt thereof, wherein:
   X is S, Y is CR⁵, and Z is CR⁶; or X is CR⁴, Y is CR⁵, and Z is S;
   A is chosen from —CH₂— and —O—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₃ alkyl optionally substituted by one NH₂, CF₃, halogen, CN, phenyl, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, phenyl, or CF₃;
   c) if A is —CH₂—, Z is S, and R¹, R², R³ and R⁴ are H, then R⁵ is not H, CN, Cl, or Br;
   d) if A is —CH₂—, Z is S, and R¹, R³ and R⁴ are H, and R⁵ is H, Cl, or Br, then R² is not methyl; and
e) the compound of Formula I is not or

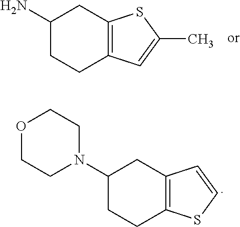

Also provided is a compound of Formula I:

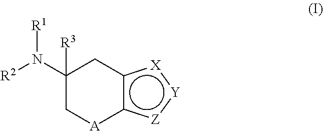

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —CH₂—;
   R¹ is chosen from H, methyl, and ethyl, and R² is chosen from H, C₁-C₃ alkyl optionally substituted by one C₃-C₅ saturated cycloalkyl, 3-5 membered saturated heterocycloalkyl, and C₃-C₅ saturated cycloalkyl optionally substituted by one or more halogen, or R¹ and R², taken together with the intervening nitrogen atom, form a 4-6 membered saturated heterocyclic ring;
   R³ is H or C₁-C₃ alkyl;
   R⁴, R⁵, and R⁶ are independently chosen from H, C₁-C₄ alkyl optionally substituted by one NR¹⁰R¹¹, C₁-C₄ haloalkyl, halogen, CN, phenyl, and C₃-C₅ saturated cycloalkyl; and R¹⁰ and R¹¹ are independently chosen from H and C₁-C₄ alkyl;
provided that:
   a) R² is H only if R¹ is H;
   b) if X is S, and R¹, R², R³ and R⁵ are H, then R⁶ is not H, methyl, CN, cyclopentyl, isobutyl, t-butyl, phenyl, CF₃, or CH₂CH₂N(CH₃)₂;
   c) if Y is S, and R¹, R², R³ and R⁶ are H, then R⁴ is not H or Br;
   d) if Z is S, and R¹, R², R³ and R⁴ are H, then R⁵ is not H, CN, Cl, or Br;
   e) if Z is S, and R¹, R³ and R⁴ are H, and R⁵ is H, Cl, or Br, then R² is not methyl; and
   f) the compound of Formula I is not

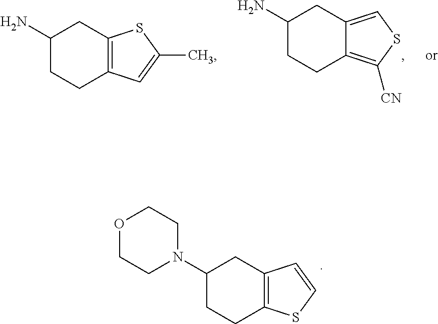 [SMILES: Cc1cc2c(s1)CC(N)CC2 ; N#Cc1scc2c1CCC(N)C2 ; c1cc2c(s1)CCC(N1CCOCC1)C2]

Also provided is a compound of Formula I:

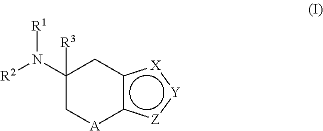

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —CH₂—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, phenyl, and C₃-C₅ saturated cycloalkyl;
provided that:
   a) R² is H only if R¹ is H;
   b) if X is S, and R¹, R², R³ and R⁵ are H, then R⁶ is not H, methyl, CN, cyclopentyl, isobutyl, t-butyl, phenyl, or CF₃;
   c) if Y is S, and R¹, R², R³ and R⁶ are H, then R⁴ is not H or Br;
d) if Z is S, and R¹, R², R³ and R⁴ are H, then R⁵ is not H, CN, Cl, or Br;
   e) if Z is S, and R¹, R³ and R⁴ are H, and R⁵ is H, Cl, or Br, then R² is not methyl; and
   f) the compound of Formula I is not

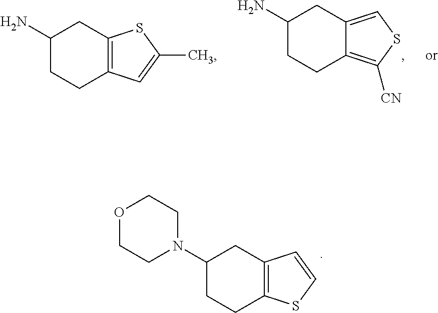 [SMILES: Cc1cc2c(s1)CC(N)CC2 ; N#Cc1scc2c1CCC(N)C2 ; c1cc2c(s1)CCC(N1CCOCC1)C2]

Also provided is a compound of Formula I:

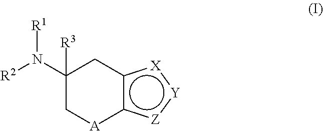

or a pharmaceutically acceptable salt thereof, wherein:
   X is S, Y is CR⁵, and Z is CR⁶; or X is CR⁴, Y is CR⁵, and Z is S;
   A is —CH₂—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, phenyl, and C₃-C₅ saturated cycloalkyl;
provided that:
   a) R² is H only if R¹ is H;
   b) if X is S, and R¹, R², R³ and R⁵ are H, then R⁶ is not H, methyl, CN, cyclopentyl, isobutyl, t-butyl, phenyl, or CF₃;
   c) if Z is S, and R¹, R², R³ and R⁴ are H, then R⁵ is not H, CN, Cl, or Br;
   d) if Z is S, and R¹, R³ and R⁴ are H, and R⁵ is H, Cl, or Br, then R² is not methyl; and
      e) the compound of Formula I is not or

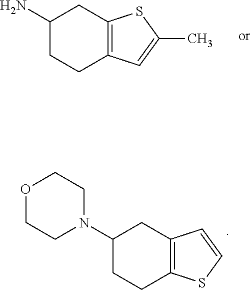

Provided is a compound of Formula I:

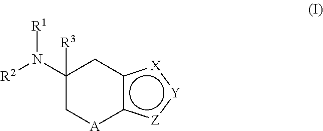

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —CH₂—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₃ alkyl, CF₃, halogen, CN, and C₃-C₅ saturated cycloalkyl;
provided that:
   a) R² is H only if R¹ is H;
   b) if A is —CH₂—, X is S, and R¹, R², R³ and R⁵ are H, then R⁶ is not H, methyl, CN, cyclopentyl, or CF₃;
   c) if Y is S, and R¹, R², R³ and R⁶ are H, then R⁴ is not H or Br;
d) if Z is S, and R¹, R², R³ and R⁴ are H, then R⁵ is not H, CN, Cl, or Br;
   e) if Z is S, and R¹, R³ and R⁴ are H, and R⁵ is H, Cl, or Br, then R² is not methyl; and
f) the compound of Formula I is not

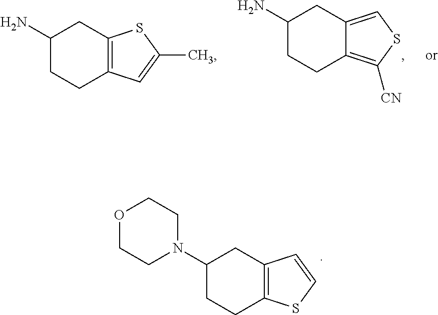 [SMILES: Cc1cc2c(s1)CC(N)CC2 ; N#Cc1scc2c1CCC(N)C2 ; c1cc2c(s1)CCC(N1CCOCC1)C2]

Also provided is a compound of Formula I:

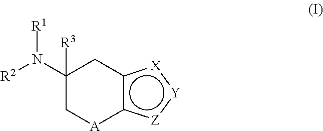

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —CH₂—;
   R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₃ alkyl, CF₃, halogen, CN, and cyclopropyl;
provided that:
   a) R² is H only if R¹ is H;
   b) if X is S, and R¹, R², R³ and R⁵ are H, then R⁶ is not H, methyl, CN, or CF₃;
   c) if Y is S, and R¹, R², R³ and R⁶ are H, then R⁴ is not H or Br;
   d) if Z is S, and R¹, R², R³ and R⁴ are H, then R⁵ is not H, CN, Cl, or Br;
   e) if Z is S, and R¹, R³ and R⁴ are H, and R⁵ is H, Cl, or Br, then R² is not methyl; and
   f) the compound of Formula I is not

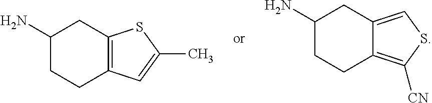

Also provided is a compound of Formula I:

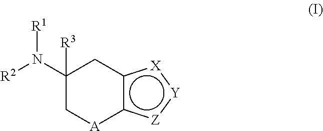

or a pharmaceutically acceptable salt thereof, wherein:
   X is S, Y is CR⁵, and Z is CR⁶; or X is CR⁴, Y is CR⁵, and Z is S;
   A is —CH₂—;
   R¹, R² and R³ are independently chosen from H and methyl; and
   R⁴, R⁵, and R⁶ are independently chosen from H, C₁-C₃ alkyl optionally substituted by one NH₂, CF₃, halogen, CN, phenyl, and C₃-C₅ saturated cycloalkyl;
provided that:
   a) R² is H only if R¹ is H;
   b) if X is S, and R¹, R², R³ and R⁵ are H, then R⁶ is not H, methyl, CN, cyclopentyl, isobutyl, t-butyl, phenyl, CF₃, or CH₂CH₂N(CH₃)₂;
   c) if Z is S, and R¹, R², R³ and R⁴ are H, then R⁵ is not H, CN, Cl, or Br;
   d) if Z is S, and R¹, R³ and R⁴ are H, and R⁵ is H, Cl, or Br, then R² is not methyl; and
e) the compound of Formula I is not

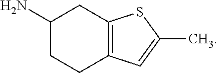 [SMILES: Cc1cc2c(s1)CC(N)CC2]

Also provided is a compound of Formula I:

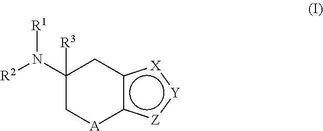

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —O—;
   R¹ is chosen from H, methyl, and ethyl, and R² is chosen from H, C₁-C₃ alkyl optionally substituted by one C₃-C₅ saturated cycloalkyl, 3-5 membered saturated heterocycloalkyl, and C₃-C₅ saturated cycloalkyl optionally substituted by one or more halogen, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, and C₃-C₅ saturated cycloalkyl;
provided that R² is H only if R¹ is H.

Also provided is a compound of Formula I:

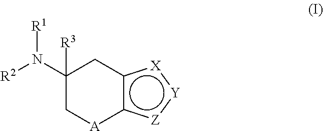

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —O—;
   R¹ is chosen from H, methyl, and ethyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, and C₃-C₅ saturated cycloalkyl;
provided that R² is H only if R¹ is H.

Also provided is a compound of Formula I:

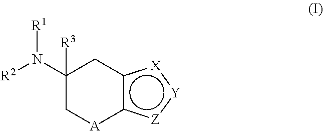

or a pharmaceutically acceptable salt thereof, wherein:
   X is S, Y is CR⁵, and Z is CR⁶; or X is CR⁴, Y is CR⁵, and Z is S;
   A is —O—;
   R¹ is chosen from H, methyl, and ethyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, and C₃-C₅ saturated cycloalkyl;
provided that R² is H only if R¹ is H.

Also provided is a compound of Formula I:

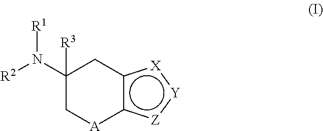

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —O—;
   R¹ is chosen from H, methyl, and ethyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring;
   R³ is H or C₁-C₃ alkyl; and
   R⁴, R⁵, and R⁶ are independently chosen from H, C₁-C₃ alkyl, CF₃, halogen, CN, and C₃-C₅ saturated cycloalkyl;
provided that R² is H only if R¹ is H.

Also provided is a compound of Formula I:

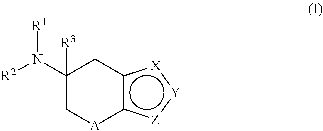

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   A is —O—;
   R¹ is chosen from H, methyl, and ethyl, and R² is chosen from H, cyclopropyl, and C₁-C₂ alkyl optionally substituted by one C₃-C₅ saturated cycloalkyl, or R¹ and R², taken together with the intervening nitrogen atom, form a pyrrolidinyl ring;
   R³ is H or C₁-C₃ alkyl; and
   R⁴, R⁵, and R⁶ are independently chosen from H, C₁-C₃ alkyl, CF₃, halogen, CN, and C₃-C₅ saturated cycloalkyl;
provided that R² is H only if R¹ is H.

Also provided is a compound of Formula I:

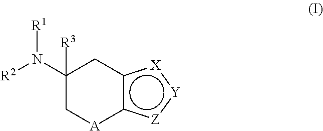

or a pharmaceutically acceptable salt thereof, wherein:
   X is S, Y is CR⁵, and Z is CR⁶; or X is CR⁴, Y is CR⁵, and Z is S;
   A is —O—;
   R¹ is chosen from H, methyl, and ethyl, and R² is chosen from H, cyclopropyl, and C₁-C₂ alkyl optionally substituted by one C₃-C₅ saturated cycloalkyl, or R¹ and R², taken together with the intervening nitrogen atom, form a pyrrolidinyl ring;
   R³ is H or C₁-C₃ alkyl; and
   R⁴, R⁵, and R⁶ are independently chosen from H, C₁-C₃ alkyl, CF₃, halogen, CN, and C₃-C₅ saturated cycloalkyl;
provided that R² is H only if R¹ is H.

In some embodiments,
1. if A is —CH₂—, X is S, and R² is C₁-C₃ alkyl optionally substituted by C₃-C₅ saturated cycloalkyl, then at least one of R¹, R³, R⁵ and R⁶ is not H; and
2. if A is —CH₂—, Z is S, and R² is C₁-C₃ alkyl optionally substituted by C₃-C₅ saturated cycloalkyl, then at least one of R¹, R³, R⁴ and R⁵ is not H.

In some embodiments, if A is —CH₂— and X is S, then at least one of R¹, R³, R⁵ and R⁶ is not H.

In some embodiments, if A is —CH₂— and Z is S, then at least one of R¹, R³, R⁴ and R⁵ is not H.

In some embodiments, X is S.

In some embodiments, X is S and R⁶ is not H.

In some embodiments, X is S, R⁵ is H, and R⁶ is not H.

In some embodiments, Y is S.

In some embodiments, Y is S and R⁴ is not H.

In some embodiments, Y is S and R⁶ is not H.

In some embodiments, Z is S.

In some embodiments, Z is S and R⁴ is not H.

In some embodiments, Z is S, R⁵ is H, and R⁴ is not H.

In some embodiments, A is chosen from —CH₂— and —O—.

In some embodiments, A is —CH₂—.

In some embodiments, A is —O—.

In some embodiments, A is a single bond.

In some embodiments, A is CH₂—CH₂.

In some embodiments, A is CH═CH.

In some embodiments, R¹ is chosen from H and methyl, and R² is chosen from H, cyclopropyl, methyl optionally substituted by cyclopropyl, ethyl,

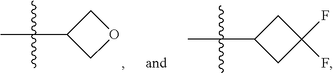 [SMILES: *C1COC1 ; *C1CC(F)(F)C1]

or R¹ and R², taken together with the intervening nitrogen atom, form a pyrrolidinyl or morpholinyl ring.

In some embodiments, R¹ is chosen from H and methyl, and R² is chosen from H, cyclopropyl, methyl optionally substituted by cyclopropyl, and ethyl, or R¹ and R², taken together with the intervening nitrogen atom, form a 4-6 membered saturated heterocyclic ring.

In some embodiments, R¹ is chosen from H and methyl, and R² is chosen from H, cyclopropyl, methyl optionally substituted by cyclopropyl, and ethyl, or R¹ and R², taken together with the intervening nitrogen atom, form a pyrrolidinyl ring.

In some embodiments, R¹ is chosen from H and methyl, and R² is chosen from H, cyclopropyl, and methyl optionally substituted by cyclopropyl, or R¹ and R², taken together with the intervening nitrogen atom, form a 4-6 membered saturated heterocyclic ring.

In some embodiments, R¹ is chosen from H and methyl, and R² is chosen from H, cyclopropyl, and methyl optionally substituted by cyclopropyl, or R¹ and R², taken together with the intervening nitrogen atom, form a pyrrolidinyl ring.

In some embodiments, R¹ and R² are chosen from H and methyl, or R¹ and R², taken together with the intervening nitrogen atom, form a 4-6 membered saturated heterocyclic ring.

In some embodiments, R¹ and R² are chosen from H and methyl, or R¹ and R², taken together with the intervening nitrogen atom, form a pyrrolidinyl ring.

In some embodiments, R¹ is H or methyl.

In some embodiments, R¹ is H.

In some embodiments, R¹ and R² are H.

In some embodiments, R² is H or methyl.

In some embodiments, R² is methyl.

In some embodiments, R¹ is H, and R² is methyl.

In some embodiments, R¹ is methyl, and R² is methyl.

In some embodiments, R³ is H or CH₃.

In some embodiments, R³ is H.

In some embodiments, R³ is C₁-C₃ alkyl.

In some embodiments, R³ is CH₃.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a 4-6 membered saturated heterocyclic ring, and R³ is C₁-C₃ alkyl.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a pyrrolidine ring, and R³ is C₁-C₃ alkyl.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a 4-6 membered saturated heterocyclic ring, and R³ is CH₃.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a pyrrolidine ring, and R³ is CH₃.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a 4-6 membered saturated heterocyclic ring, and R³ is H.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a pyrrolidine ring, and R³ is H.

In some embodiments, R¹ is chosen from H and methyl, R² is chosen from H, cyclopropyl, and methyl optionally substituted by cyclopropyl, or R¹ and R², taken together with the intervening nitrogen atom, form a 4-6 membered saturated heterocyclic ring, and R³ is C₁-C₃ alkyl.

In some embodiments, R¹ is chosen from H and methyl, R² is chosen from H, cyclopropyl, and methyl optionally substituted by cyclopropyl, or R¹ and R², taken together with the intervening nitrogen atom, form a pyrrolidine ring, and R³ is C₁-C₃ alkyl.

In some embodiments, R¹ is chosen from H and methyl, R² is chosen from H, cyclopropyl, and methyl optionally substituted by cyclopropyl, or R¹ and R², taken together with the intervening nitrogen atom, form a 4-6 membered saturated heterocyclic ring, and R³ is CH₃.

In some embodiments, R¹ is chosen from H and methyl, R² is chosen from H, cyclopropyl, and methyl optionally substituted by cyclopropyl, or R¹ and R², taken together with the intervening nitrogen atom, form a pyrrolidine ring, and R³ is CH₃.

In some embodiments, R¹ is chosen from H and methyl, R² is chosen from H, cyclopropyl, and methyl optionally substituted by cyclopropyl, or R¹ and R², taken together with the intervening nitrogen atom, form a 4-6 membered saturated heterocyclic ring, and R³ is H.

In some embodiments, R¹ is chosen from H and methyl, R² is chosen from H, cyclopropyl, and methyl optionally substituted by cyclopropyl, or R¹ and R², taken together with the intervening nitrogen atom, form a pyrrolidine ring, and R³ is H.

In some embodiments, R¹ and R² are chosen from H and methyl, or R¹ and R², taken together with the intervening nitrogen atom, form a 4-6 membered saturated heterocyclic ring, and R³ is C₁-C₃ alkyl.

In some embodiments, R¹ and R² are chosen from H and methyl, or R¹ and R², taken together with the intervening nitrogen atom, form a pyrrolidine ring, and R³ is C₁-C₃ alkyl.

In some embodiments, R¹ and R² are chosen from H and methyl, or R¹ and R², taken together with the intervening nitrogen atom, form a 4-6 membered saturated heterocyclic ring, and R³ is CH₃.

In some embodiments, R¹ and R² are chosen from H and methyl, or R¹ and R², taken together with the intervening nitrogen atom, form a pyrrolidine ring, and R³ is CH₃.

In some embodiments, R¹ and R² are chosen from H and methyl, or R¹ and R², taken together with the intervening nitrogen atom, form a 4-6 membered saturated heterocyclic ring, and R³ is H.

In some embodiments, R¹ and R² are chosen from H and methyl, or R¹ and R², taken together with the intervening nitrogen atom, form a pyrrolidine ring, and R³ is H.

In some embodiments, R¹ is H, and R³ is H.

In some embodiments, R¹ is H, and R³ is methyl.

In some embodiments, R¹ is H, R² is methyl, and R³ is H.

In some embodiments, R¹, R², and R³ are H.

In some embodiments, R¹ is H, R² is methyl, and R³ is methyl.

In some embodiments, R¹ is H, R² is H, and R³ is methyl.

In some embodiments, R⁴, R⁵, and R⁶ are independently chosen from H, methyl, ethyl, aminomethyl, CF₃, halogen, CN, phenyl, cyclopropyl, and cyclobutyl.

In some embodiments, R⁴, R⁵, and R⁶ are independently chosen from H, methyl, ethyl, aminomethyl, CF₃, F, Cl, Br, CN, phenyl, cyclopropyl, and cyclobutyl.

In some embodiments, R⁴, R⁵, and R⁶ are independently chosen from H, methyl, ethyl, CF₃, halogen, CN, cyclopropyl, and cyclobutyl.

In some embodiments, R⁴, R⁵, and R⁶ are independently chosen from H, methyl, ethyl, aminomethyl, CF₃, CN, phenyl, cyclopropyl, and cyclobutyl.

In some embodiments, R⁴ is chosen from H, methyl, ethyl, aminomethyl, CF₃, halogen, CN, phenyl, cyclopropyl, and cyclobutyl; R⁵ is chosen from H, methyl, halogen, and cyclopropyl; and R⁶ is chosen from H, methyl, CF₃, halogen, and cyclopropyl.

In some embodiments, R⁴ is chosen from H, methyl, ethyl, aminomethyl, CF₃, F, Cl, Br, CN, phenyl, cyclopropyl, and cyclobutyl; R⁵ is chosen from H, methyl, F, Cl, and cyclopropyl; and R⁶ is chosen from H, methyl, CF₃, F, Cl, and cyclopropyl.

In some embodiments, R⁴ is chosen from H, methyl, ethyl, CF₃, halogen, CN, and cyclopropyl; R⁵ is chosen from H, methyl, halogen, and cyclopropyl; and R⁶ is chosen from H, methyl, CF₃, halogen, and cyclopropyl.

In some embodiments, R⁴ is chosen from H, methyl, ethyl, aminomethyl, CF₃, CN, phenyl, cyclopropyl, and cyclobutyl; R⁵ is chosen from H, methyl, and cyclopropyl; and R⁶ is chosen from H, methyl, CF₃, and cyclopropyl.

In some embodiments, R⁴ is chosen from H, methyl, ethyl, aminomethyl, CF₃, halogen, CN, phenyl, cyclopropyl, and cyclobutyl.

In some embodiments, R⁴ is chosen from H, methyl, ethyl, CF₃, halogen, CN, cyclopropyl, and cyclobutyl.

In some embodiments, R⁴ is chosen from H, methyl, ethyl, CF₃, halogen, and cyclopropyl.

In some embodiments, R⁴ is chosen from H, methyl, CF₃, halogen, and cyclopropyl.

In some embodiments, R⁴ is chosen from H, methyl, CF₃, and cyclopropyl.

In some embodiments, R⁵ is chosen from H, C₁-C₃ alkyl, halogen, and cyclopropyl.

In some embodiments, R⁵ is chosen from H, methyl, halogen, and cyclopropyl.

In some embodiments, R⁵ is chosen from H, methyl, F, Cl, and cyclopropyl.

In some embodiments, R⁵ is chosen from H, C₁-C₃ alkyl, and cyclopropyl.

In some embodiments, R⁵ is chosen from H, methyl, and cyclopropyl.

In some embodiments, R⁵ is chosen from H and methyl.

In some embodiments, R⁵ is H.

In some embodiments, R⁵ is methyl.

In some embodiments, R⁶ is chosen from H, methyl, CF₃, halogen, and cyclopropyl.

In some embodiments, R⁶ is chosen from H, methyl, CF₃, and cyclopropyl.

In some embodiments, R⁴, R⁵, and R⁶ are independently chosen from H, C₁-C₃ alkyl, C₁-C₂ fluoroalkyl, and cyclopropyl.

In some embodiments, R⁴, R⁵, and R⁶ are independently chosen from H, C₁-C₃ alkyl, CF₃, and cyclopropyl.

In some embodiments, at least one of R⁴, R⁵, and R⁶ is H.

In some embodiments, at least one of R⁴, R⁵, and R⁶ is F.

In some embodiments, R⁴ is F.

In some embodiments, R⁵ is F.

In some embodiments, R⁶ is F.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a 4-6 membered saturated heterocyclic ring, R³ is C₁-C₃ alkyl, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a pyrrolidine ring, R³ is C₁-C₃ alkyl, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a 4-6 membered saturated heterocyclic ring, R³ is CH₃, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a pyrrolidine ring, R³ is CH₃, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a 4-6 membered saturated heterocyclic ring, R³ is H, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, methyl optionally substituted by cyclopropyl, and ethyl, or R¹ and R², taken together with the intervening nitrogen atom, form a pyrrolidine ring, R³ is H, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, and methyl optionally substituted by cyclopropyl, or R¹ and R², taken together with the intervening nitrogen atom, form a 4-6 membered saturated heterocyclic ring, R³ is C₁-C₃ alkyl, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, and methyl optionally substituted by cyclopropyl, or R¹ and R², taken together with the intervening nitrogen atom, form a pyrrolidine ring, R³ is C₁-C₃ alkyl, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, and methyl optionally substituted by cyclopropyl, or R¹ and R², taken together with the intervening nitrogen atom, form a 4-6 membered saturated heterocyclic ring, R³ is CH₃, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, and methyl optionally substituted by cyclopropyl, or R¹ and R², taken together with the intervening nitrogen atom, form a pyrrolidine ring, R³ is CH₃, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, and methyl optionally substituted by cyclopropyl, or R¹ and R², taken together with the intervening nitrogen atom, form a 4-6 membered saturated heterocyclic ring, R³ is H, and R⁴, R⁵, and R⁶ are independently chosen from H, C₁-C₃ alkyl, C₁-C₃ haloalkyl, halogen, CN, and cyclopropyl.

In some embodiments, R¹ is chosen from H and methyl, R² is chosen from H, cyclopropyl, and methyl optionally substituted by cyclopropyl, or R¹ and R², taken together with the intervening nitrogen atom, form a pyrrolidine ring, R³ is H, and R⁴, R⁵, and R⁶ are independently chosen from H, C₁-C₃ alkyl, C₁-C₃ haloalkyl, halogen, CN, and cyclopropyl.

In some embodiments, R¹ and R² are chosen from H and methyl, or R¹ and R², taken together with the intervening nitrogen atom, form a 4-6 membered saturated heterocyclic ring, R³ is C₁-C₃ alkyl, and R⁴, R⁵, and R⁶ are independently chosen from H, C₁-C₃ alkyl, C₁-C₃ haloalkyl, halogen, CN, and cyclopropyl.

In some embodiments, R¹ and R² are chosen from H and methyl, or R¹ and R², taken together with the intervening nitrogen atom, form a pyrrolidine ring, R³ is C₁-C₃ alkyl, and R⁴, R⁵, and R⁶ are independently chosen from H, C₁-C₃ alkyl, C₁-C₃ haloalkyl, halogen, CN, and cyclopropyl.

In some embodiments, R¹ and R² are chosen from H and methyl, or R¹ and R², taken together with the intervening nitrogen atom, form a 4-6 membered saturated heterocyclic ring, R³ is CH₃, and R⁴, R⁵, and R⁶ are independently chosen from H, C₁-C₃ alkyl, C₁-C₃ haloalkyl, halogen, CN, and cyclopropyl.

In some embodiments, R¹ and R² are chosen from H and methyl, or R¹ and R², taken together with the intervening nitrogen atom, form a pyrrolidine ring, R³ is CH₃, and R⁴, R⁵, and R⁶ are independently chosen from H, C₁-C₃ alkyl, C₁-C₃ haloalkyl, halogen, CN, and cyclopropyl.

In some embodiments, R¹ and R² are chosen from H and methyl, or R¹ and R², taken together with the intervening nitrogen atom, form a 4-6 membered saturated heterocyclic ring, R³ is H, and R⁴, R⁵, and R⁶ are independently chosen from H, C₁-C₃ alkyl, C₁-C₃ haloalkyl, halogen, CN, and cyclopropyl.

In some embodiments, R¹ and R² are chosen from H and methyl, or R¹ and R², taken together with the intervening nitrogen atom, form a pyrrolidine ring, R³ is H, and R⁴, R⁵, and R⁶ are independently chosen from H, C₁-C₃ alkyl, C₁-C₃ haloalkyl, halogen, CN, and cyclopropyl.

In some embodiments, R¹⁰ and R¹¹ are independently chosen from H and C₁-C₃ alkyl.

In some embodiments, R¹⁰ and R¹¹ are independently chosen from H and methyl.

In some embodiments, R¹⁰ is H and R¹¹ is chosen from H and C₁-C₄ alkyl.

In some embodiments, R¹⁰ is H and R¹¹ is chosen from H and C₁-C₃ alkyl.

In some embodiments, R¹⁰ is H and R¹¹ is chosen from H and methyl.

In some embodiments, R¹⁰ and R¹¹ are H.

Also provided is a compound of Formula I, wherein the compound is a compound of Formula Ia:

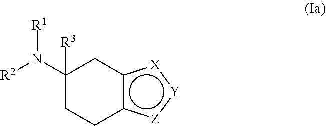

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, and C₃-C₅ saturated cycloalkyl;
provided that:
   a) if X is S, and R¹, R², R³ and R⁵ are H, then R⁶ is not H, methyl, CN, cyclopentyl, isobutyl, t-butyl, or CF₃;
   b) if Y is S, and R¹, R², R³ and R⁶ are H, then R⁴ is not H or Br;
   c) if Z is S, and R¹, R², R³ and R⁴ are H, then R⁵ is not H, CN, Cl, or Br;
   d) if Z is S, and R¹, R³ and R⁴ are H, and R⁵ is H, Cl, or Br, then R² is not methyl; and
   e) the compound of Formula Ia is not

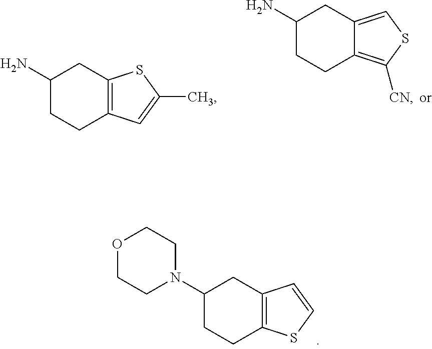 [SMILES: Cc1cc2c(s1)CC(N)CC2 ; N#Cc1scc2c1CCC(N)C2 ; c1cc2c(s1)CCC(N1CCOCC1)C2]

Also provided is a compound of Formula Ia or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula Ia or a pharmaceutically acceptable salt thereof, wherein R² and R³ are independently chosen from H and methyl.

Also provided is a compound of Formula Ia or a pharmaceutically acceptable salt thereof, wherein R⁴, R⁵, and R⁶ are independently chosen from H, C₁-C₂ alkyl, C₁-C₂ haloalkyl, halogen, CN, and C₃-C₅ saturated cycloalkyl.

Also provided is a compound of Formula I, wherein the compound is a compound of Formula Ib:

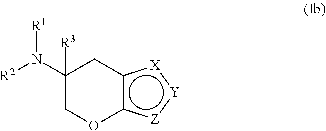

or a pharmaceutically acceptable salt thereof, wherein:
   X is S, Y is CR⁵, and Z is CR⁶; or X is CR⁴, Y is S, and Z is CR⁶; or X is CR⁴, Y is CR⁵, and Z is S;
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴, R⁵, and R⁶ are independently chosen from H, C₁-C₄ alkyl, C₁-C₄ haloalkyl, halogen, CN, and C₃-C₅ saturated cycloalkyl.

Also provided is a compound of Formula Ib or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula Ib or a pharmaceutically acceptable salt thereof, wherein R² and R³ are independently chosen from H and methyl.

Also provided is a compound of Formula Ib or a pharmaceutically acceptable salt thereof, wherein R⁴, R⁵, and R⁶ are independently chosen from H, C₁-C₂ alkyl, C₁-C₂ haloalkyl, halogen, CN, and C₃-C₅ saturated cycloalkyl.

Also provided is a compound of Formula I, wherein the compound is a compound of Formula Ic:

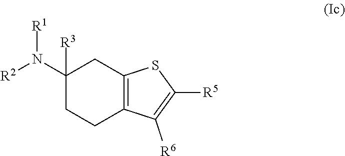

or a pharmaceutically acceptable salt thereof, wherein:
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁵ and R⁶ are independently chosen from H, C₁-C₄ alkyl, C₁-C₄ haloalkyl, halogen, CN, and C₃-C₅ saturated cycloalkyl;
provided that:
   a) if R¹, R², R³ and R⁵ are H, then R⁶ is not H, methyl, CN, cyclopentyl, isobutyl, t-butyl, or CF₃; and
   b) the compound of Formula Ic is not

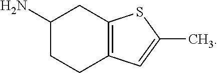 [SMILES: Cc1cc2c(s1)CC(N)CC2]

Also provided is a compound of Formula Ic or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula Ic or a pharmaceutically acceptable salt thereof, wherein R² and R³ are independently chosen from H and methyl.

Also provided is a compound of Formula Ic or a pharmaceutically acceptable salt thereof, wherein R⁵ and R⁶ are independently chosen from H, C₁-C₂ alkyl, C₁-C₂ haloalkyl, halogen, CN, and C₃-C₅ saturated cycloalkyl.

Also provided is a compound of Formula I, wherein the compound is a compound of Formula Id:

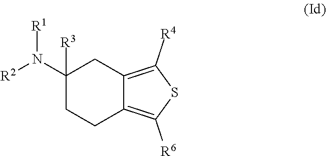

or a pharmaceutically acceptable salt thereof, wherein:
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴ and R⁶ are independently chosen from H, C₁-C₄ alkyl, C₁-C₄ haloalkyl, halogen, CN, and C₃-C₅ saturated cycloalkyl;
provided that:
   a) if R¹, R², R³ and R⁶ are H, then R⁴ is not H or Br; and
   b) the compound of Formula Id is not

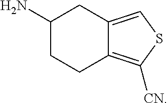 [SMILES: N#Cc1scc2c1CCC(N)C2]

Also provided is a compound of Formula Id or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula Id or a pharmaceutically acceptable salt thereof, wherein R² and R³ are independently chosen from H and methyl.

Also provided is a compound of Formula Id or a pharmaceutically acceptable salt thereof, wherein R⁴ and R⁶ are independently chosen from H, C₁-C₂ alkyl, C₁-C₂ haloalkyl, halogen, CN, and C₃-C₅ saturated cycloalkyl.

Also provided is a compound of Formula I, wherein the compound is a compound of Formula Ie:

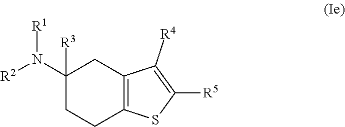

or a pharmaceutically acceptable salt thereof, wherein:
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴ and R⁵ are independently chosen from H, C₁-C₄ alkyl, C₁-C₄ haloalkyl, halogen, CN, and C₃-C₅ saturated cycloalkyl;
provided that:
   a) if R¹, R², R³ and R⁴ are H, then R⁵ is not H, CN, Cl, or Br;
   b) if R¹, R³ and R⁴ are H, and R⁵ is H, Cl, or Br, then R² is not methyl; and
   c) the compound of Formula Ie is not

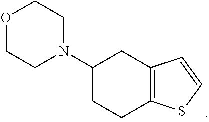 [SMILES: c1cc2c(s1)CCC(N1CCOCC1)C2]

Also provided is a compound of Formula Ie or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula Ie or a pharmaceutically acceptable salt thereof, wherein R² and R³ are independently chosen from H and methyl.

Also provided is a compound of Formula Ie or a pharmaceutically acceptable salt thereof, wherein R⁴ and R⁵ are independently chosen from H, C₁-C₂ alkyl, C₁-C₂ haloalkyl, halogen, CN, and C₃-C₅ saturated cycloalkyl.

Also provided is a compound of Formula I, wherein the compound is a compound of Formula If:

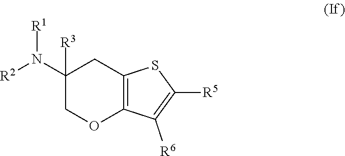

or a pharmaceutically acceptable salt thereof, wherein:
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁵ and R⁶ are independently chosen from H, C₁-C₄ alkyl, C₁-C₄ haloalkyl, halogen, CN, and C₃-C₅ saturated cycloalkyl.

Also provided is a compound of Formula If or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula If or a pharmaceutically acceptable salt thereof, wherein R² and R³ are independently chosen from H and methyl.

Also provided is a compound of Formula If or a pharmaceutically acceptable salt thereof, wherein R⁵ and R⁶ are independently chosen from H, C₁-C₂ alkyl, C₁-C₂ haloalkyl, halogen, CN, and C₃-C₅ saturated cycloalkyl.

Also provided is a compound of Formula I, wherein the compound is a compound of Formula Ig:

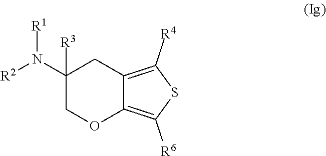

or a pharmaceutically acceptable salt thereof, wherein:
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴ and R⁶ are independently chosen from H, C₁-C₄ alkyl, C₁-C₄ haloalkyl, halogen, CN, and C₃-C₅ saturated cycloalkyl.

Also provided is a compound of Formula Ig or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula Ig or a pharmaceutically acceptable salt thereof, wherein R² and R³ are independently chosen from H and methyl.

Also provided is a compound of Formula Ig or a pharmaceutically acceptable salt thereof, wherein R⁴ and R⁶ are independently chosen from H, C₁-C₂ alkyl, C₁-C₂ haloalkyl, halogen, CN, and C₃-C₅ saturated cycloalkyl.

Also provided is a compound of Formula I, wherein the compound is a compound of Formula Ih:

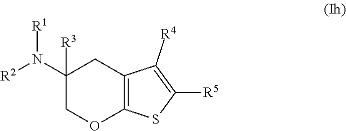

or a pharmaceutically acceptable salt thereof, wherein:
   R¹ is chosen from H and methyl, and R² is chosen from H, C₁-C₂ alkyl optionally substituted by one C₃-C₅ saturated cycloalkyl, and C₃-C₅ saturated cycloalkyl, or R¹ and R², taken together with the intervening nitrogen atom, form a 3-7 membered saturated heterocyclic ring;
   R³ is H or C₁-C₃ alkyl; and
   R⁴ and R⁵ are independently chosen from H, C₁-C₄ alkyl, C₁-C₄ haloalkyl, halogen, CN, and C₃-C₅ saturated cycloalkyl.

Also provided is a compound of Formula Ih or a pharmaceutically acceptable salt thereof, wherein R¹ is chosen from H and methyl, and R² is chosen from H, cyclopropyl, and C₁-C₂ alkyl optionally substituted by one C₃-C₅ saturated cycloalkyl, or R¹ and R², taken together with the intervening nitrogen atom, form a 4-6 membered saturated heterocyclic ring; and R³ is H or methyl.

Also provided is a compound of Formula Ih or a pharmaceutically acceptable salt thereof, wherein R² and R³ are independently chosen from H and methyl.

Also provided is a compound of Formula Ih or a pharmaceutically acceptable salt thereof, wherein R⁴ and R⁵ are independently chosen from H, C₁-C₂ alkyl, C₁-C₂ haloalkyl, halogen, CN, and C₃-C₅ saturated cycloalkyl.

In some embodiments, the compound of Formula I is chosen from

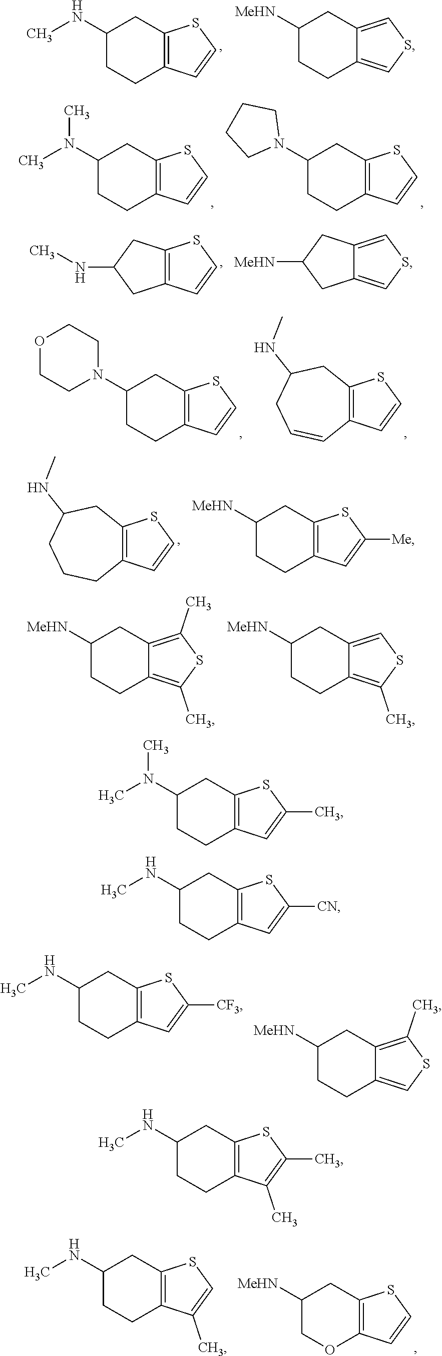

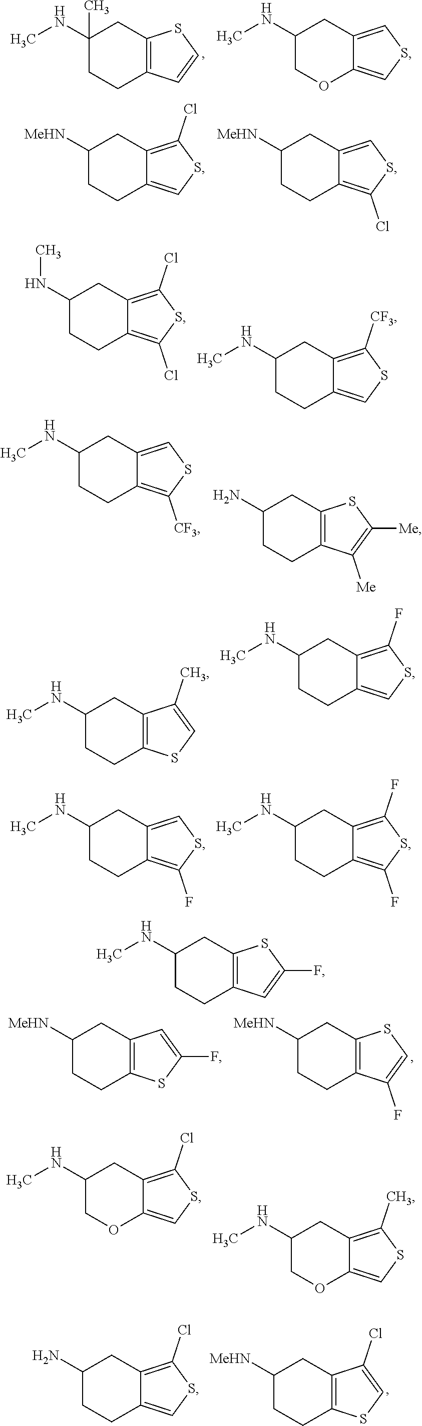

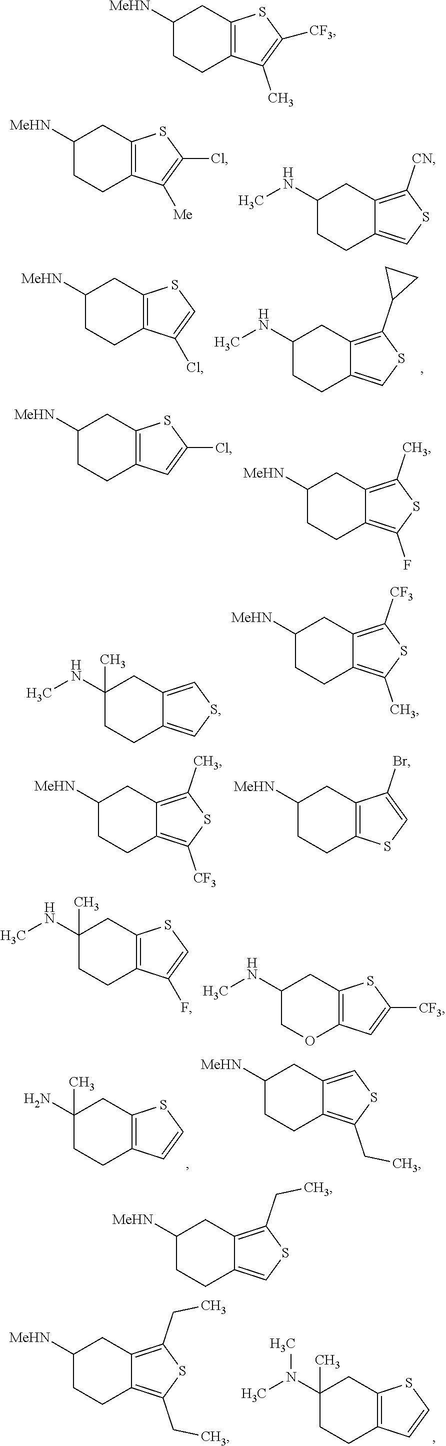

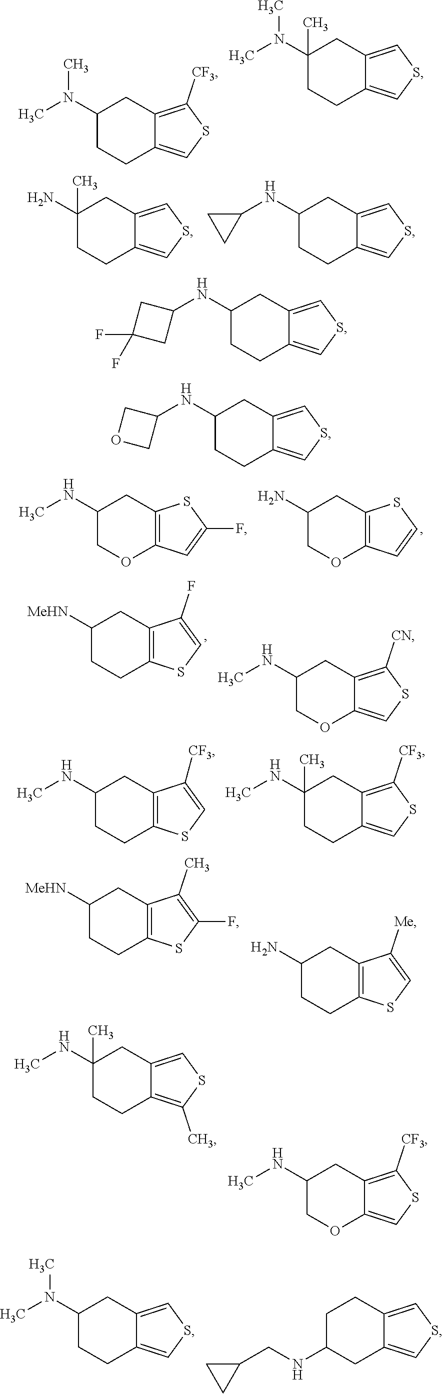

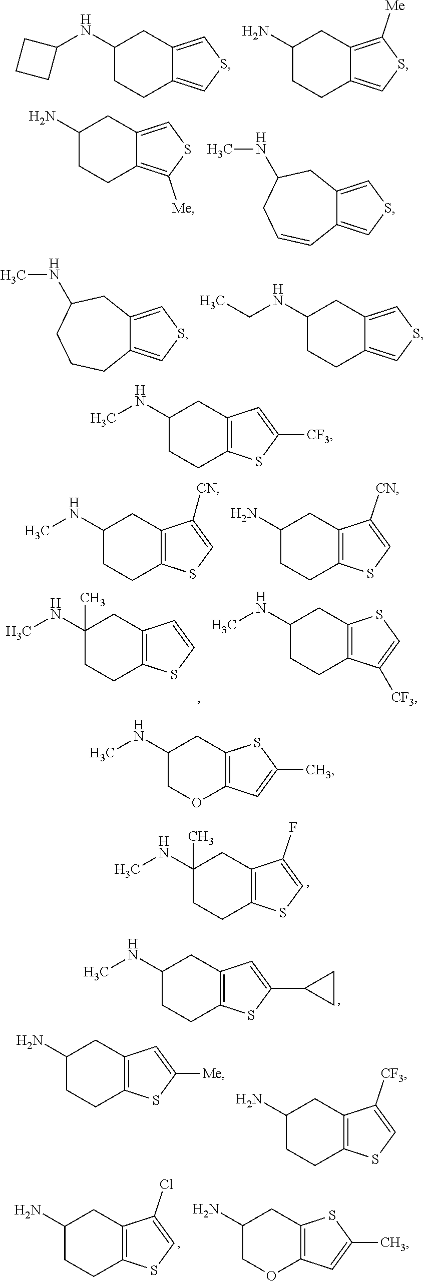

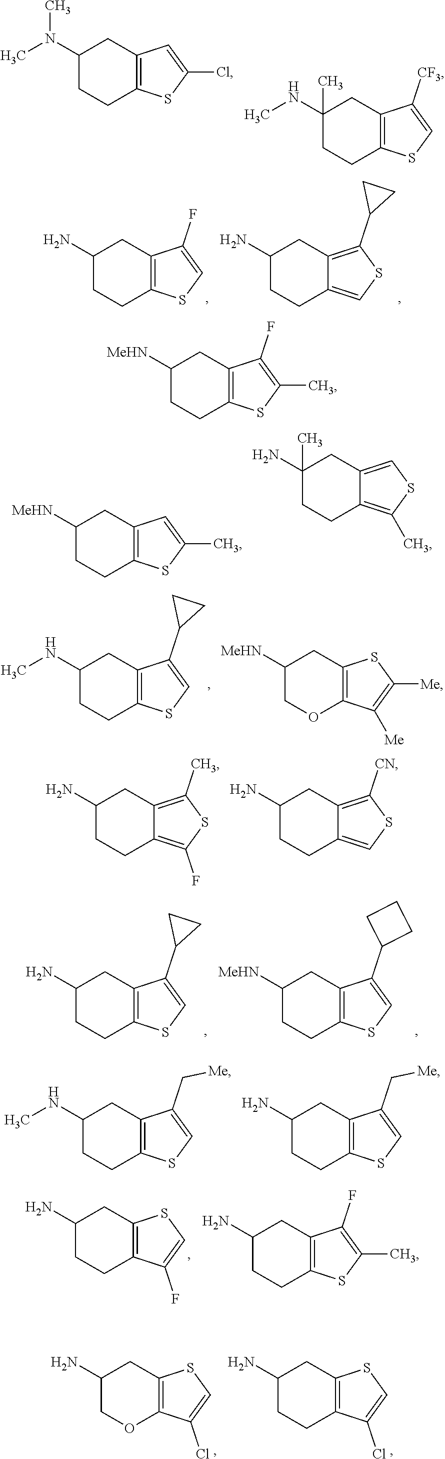

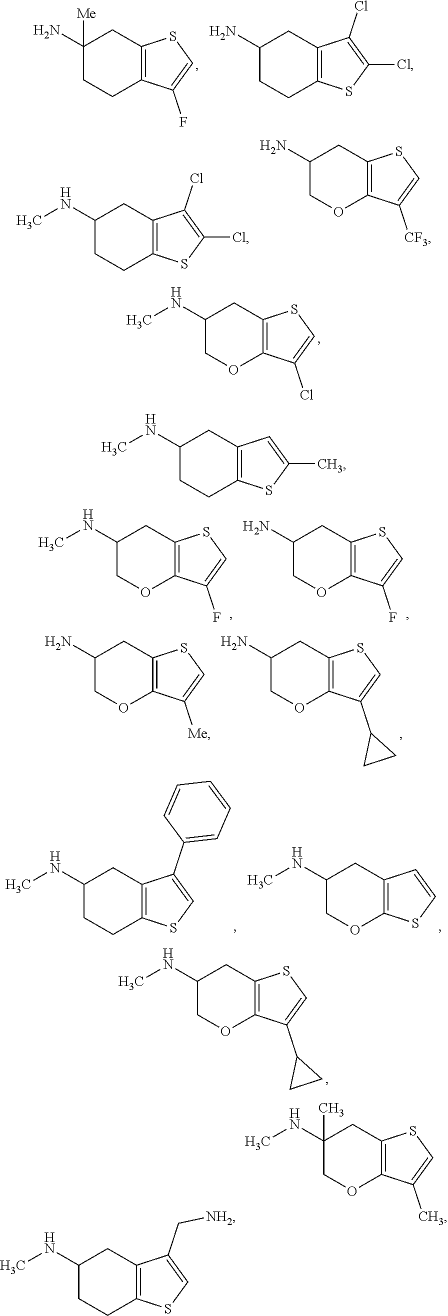

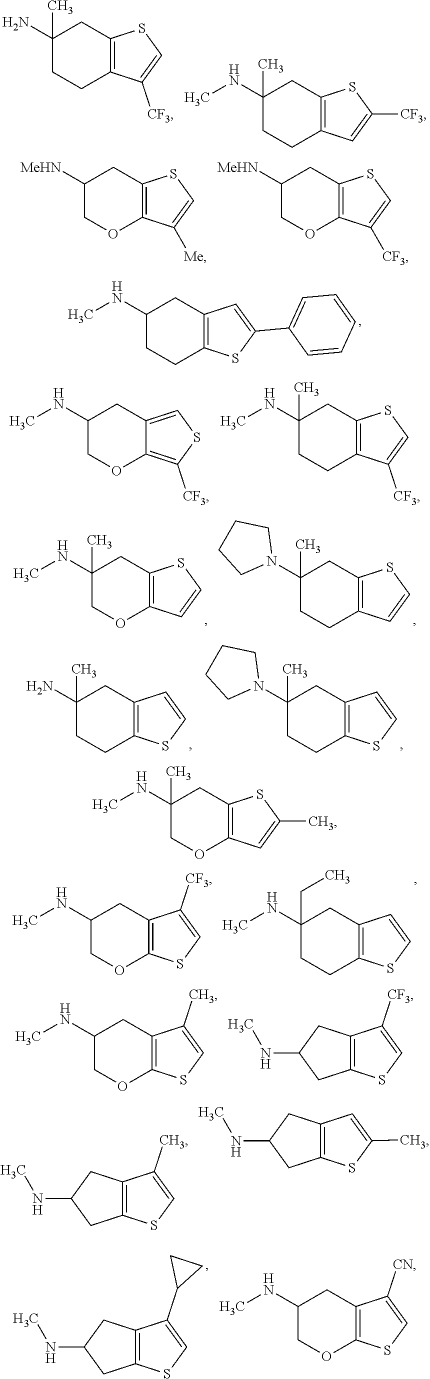

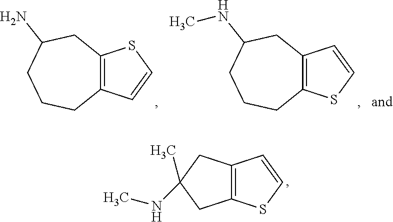

or a pharmaceutically acceptable salt thereof.

Also provided is a compound of Formula I, Formula Ia, Formula Ib, Formula Ic, Formula Id, Formula Ie, Formula If, or Formula Ig, Formula Ih, or pharmaceutically acceptable salt thereof, enriched in one enantiomer. Also provided is an isolated enantiomer of a compound, or pharmaceutically acceptable salt described herein. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 80% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 85% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 90% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 95% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 96% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 97% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 98% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 99% enantiomerically pure. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 99.5% enantiomerically pure.

In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 80% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 80% enriched in the (R) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 85% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 85% enriched in the (R) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 90% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 90% enriched in the (R) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 95% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 95% enriched in the (R) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 96% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 96% enriched in the (R) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 97% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 97% enriched in the (R) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 98% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 98% enriched in the (R) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 99% enriched in the (S) enantiomer. In some embodiments, the compound of Formula I, Formula Ia, Formula Ib, Formula Ic, Formula Id, Formula Ie, Formula If, or Formula Ig, Formula Ih, or a pharmaceutically acceptable salt thereof, is at least 99% enriched in the (R) enantiomer.

Also provided is a compound chosen from

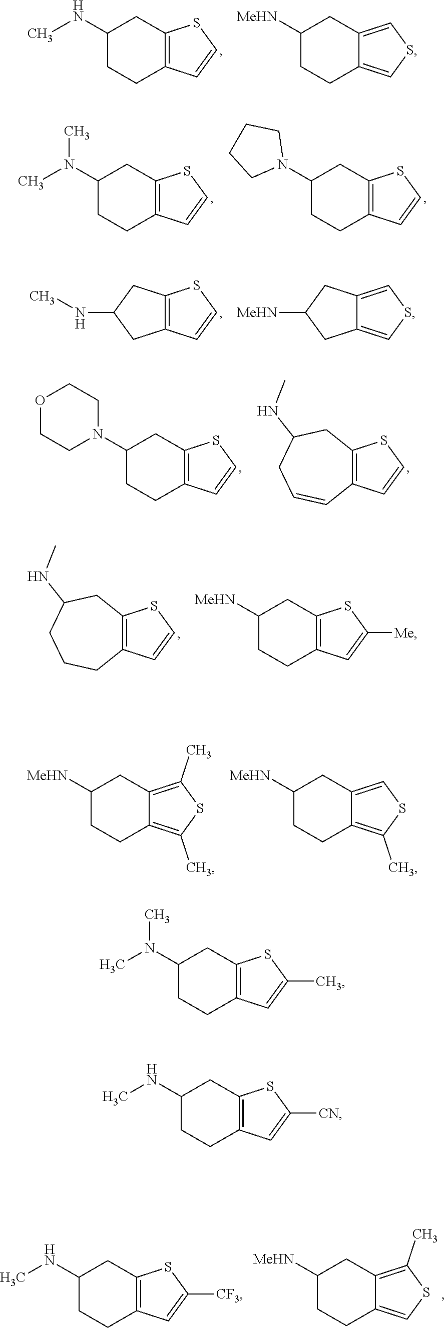

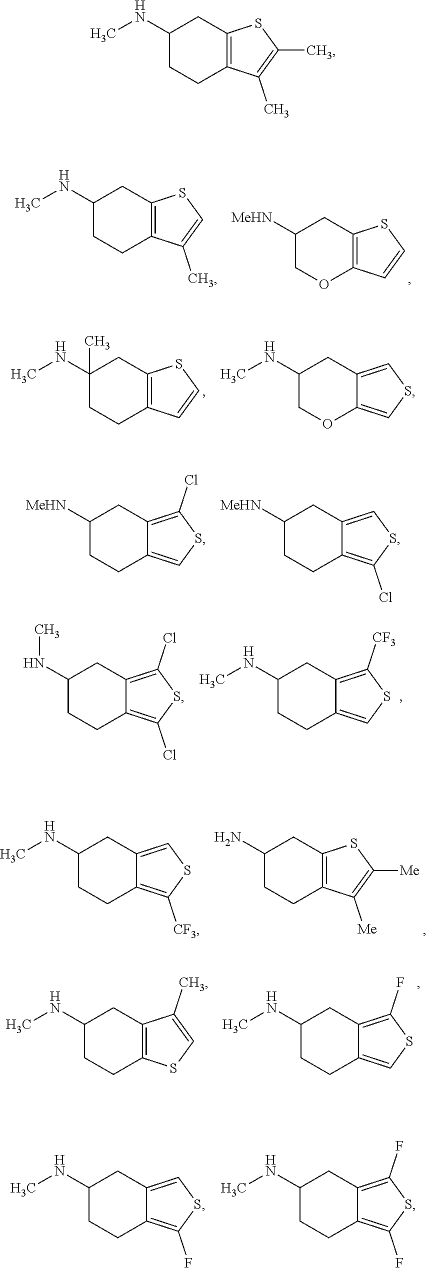

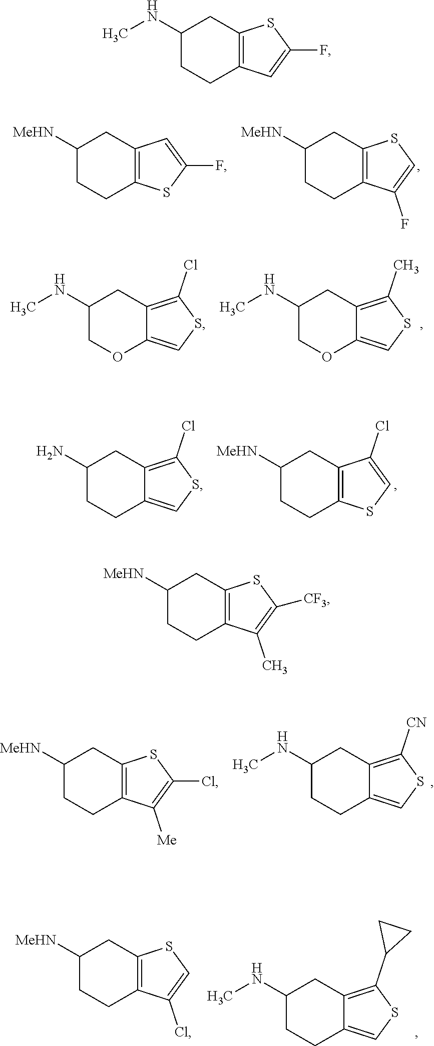

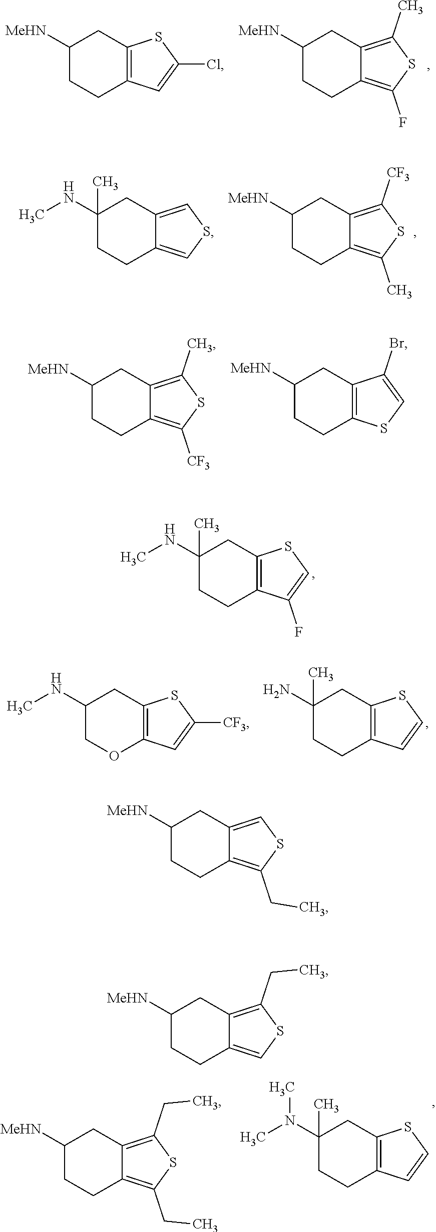

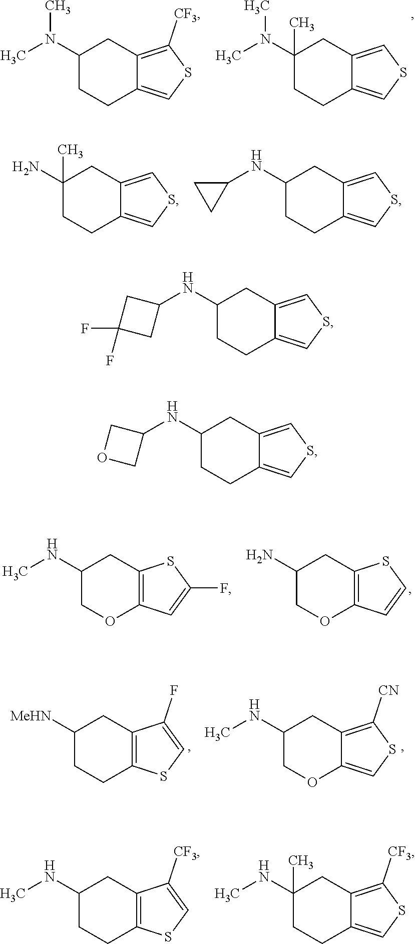

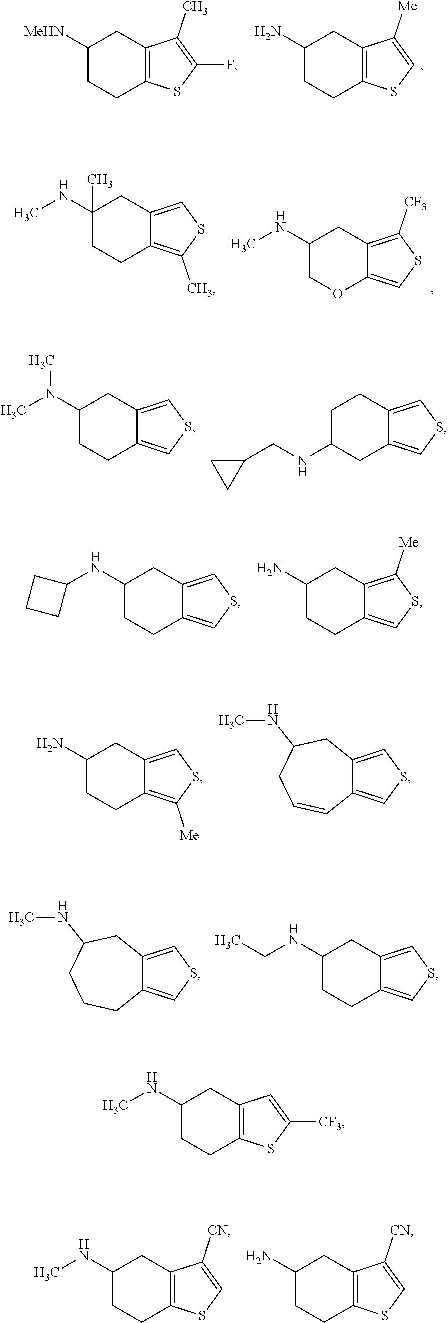

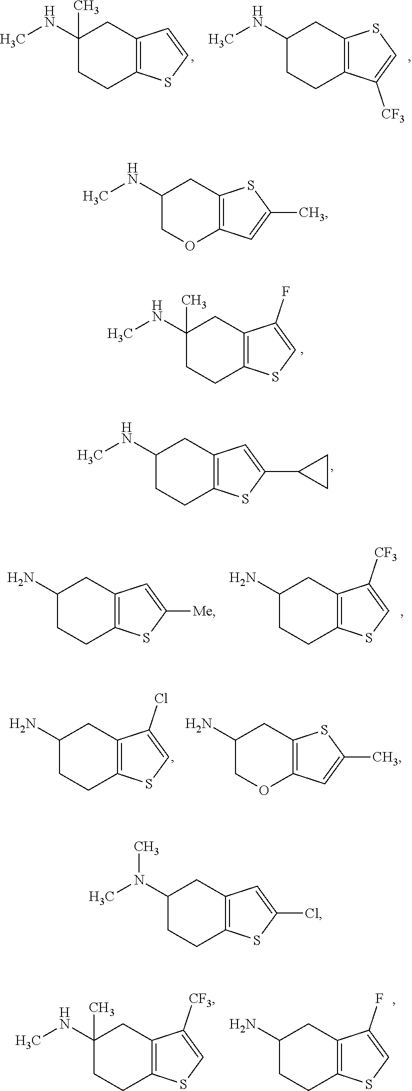

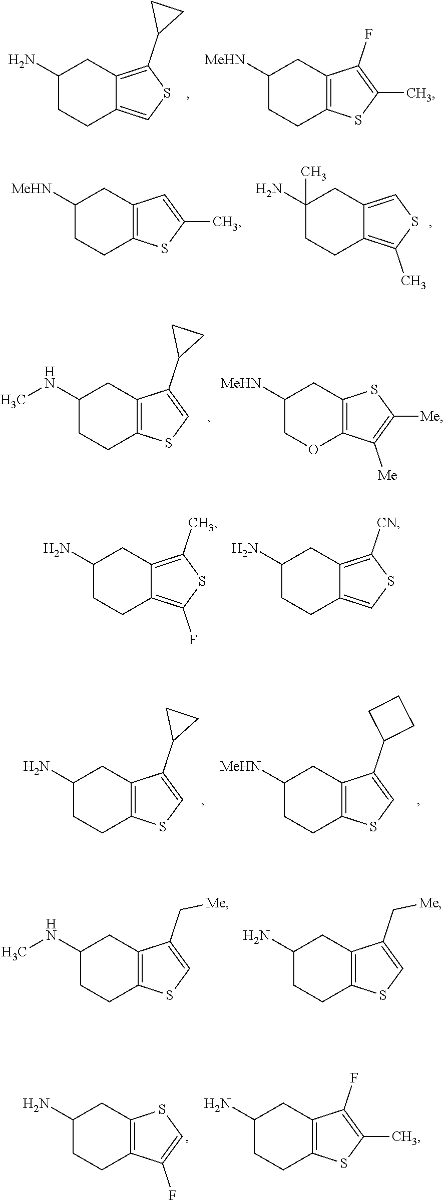

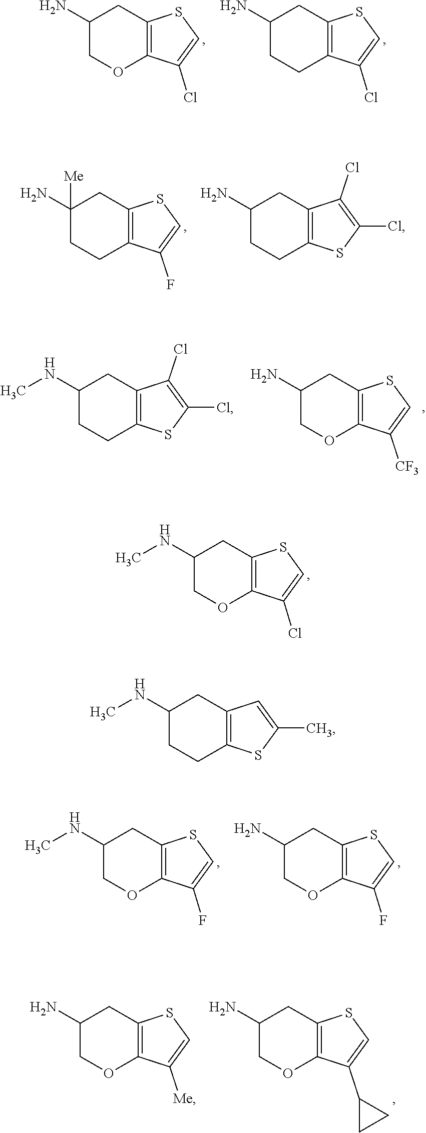

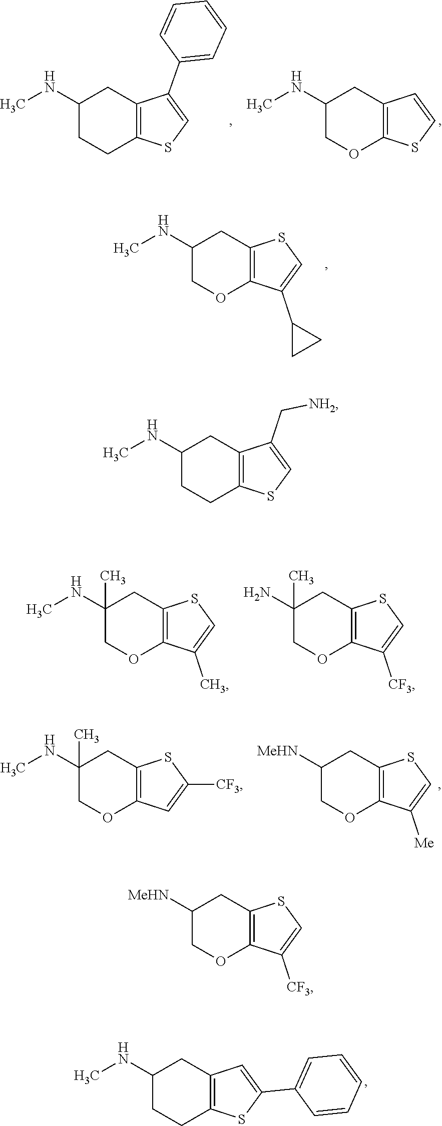

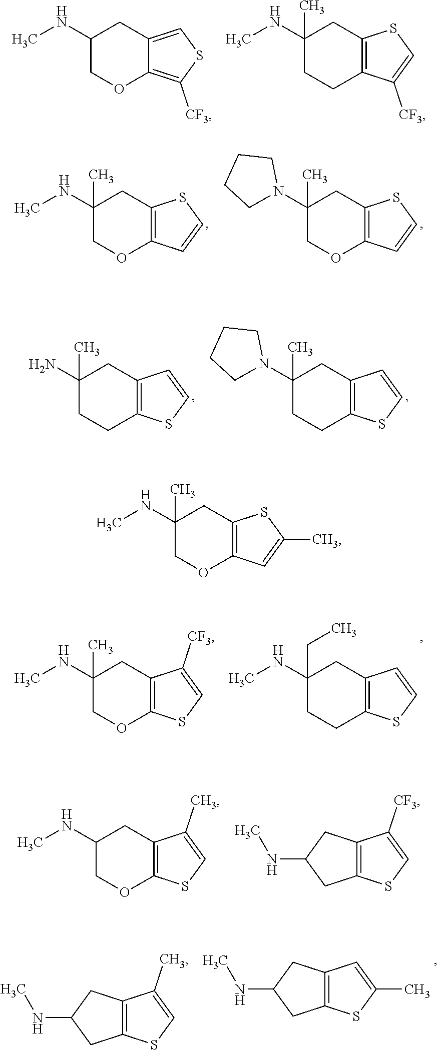

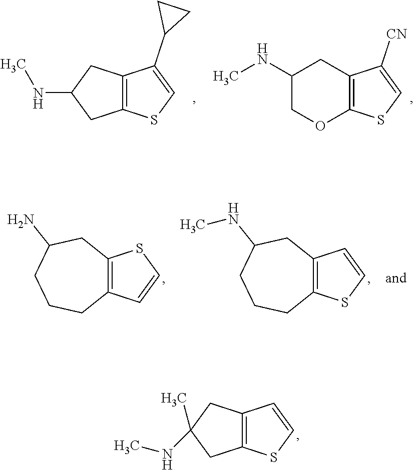

or a pharmaceutically acceptable salt thereof, wherein the compound is enriched in the (R) or (S) enantiomer. In some embodiments, the compound, or a pharmaceutically acceptable salt thereof, is at least 80% enriched in the (S) enantiomer. In some embodiments, the compound, or a pharmaceutically acceptable salt thereof, is at least 80% enriched in the (R) enantiomer. In some embodiments, the compound, or a pharmaceutically acceptable salt thereof, is at least 85% enriched in the (S) enantiomer. In some embodiments, the compound, or a pharmaceutically acceptable salt thereof, is at least 85% enriched in the (R) enantiomer. In some embodiments, the compound, or a pharmaceutically acceptable salt thereof, is at least 90% enriched in the (S) enantiomer. In some embodiments, the compound, or a pharmaceutically acceptable salt thereof, is at least 90% enriched in the (R) enantiomer. In some embodiments, the compound, or a pharmaceutically acceptable salt thereof, is at least 95% enriched in the (S) enantiomer. In some embodiments, the compound, or a pharmaceutically acceptable salt thereof, is at least 95% enriched in the (R) enantiomer. In some embodiments, the compound, or a pharmaceutically acceptable salt thereof, is at least 96% enriched in the (S) enantiomer. In some embodiments, the compound, or a pharmaceutically acceptable salt thereof, is at least 96% enriched in the (R) enantiomer. In some embodiments, the compound, or a pharmaceutically acceptable salt thereof, is at least 97% enriched in the (S) enantiomer. In some embodiments, the compound, or a pharmaceutically acceptable salt thereof, is at least 97% enriched in the (R) enantiomer. In some embodiments, the compound, or a pharmaceutically acceptable salt thereof, is at least 98% enriched in the (S) enantiomer. In some embodiments, the compound, or a pharmaceutically acceptable salt thereof, is at least 98% enriched in the (R) enantiomer. In some embodiments, the compound, or a pharmaceutically acceptable salt thereof, is at least 99% enriched in the (S) enantiomer. In some embodiments, the compound, or a pharmaceutically acceptable salt thereof, is at least 99% enriched in the (R) enantiomer.

Also provided is a composition comprising a compound described herein, or a pharmaceutically acceptable salt thereof. Also provided is a composition comprising a compound described herein, or a pharmaceutically acceptable salt thereof, wherein the compound, or a pharmaceutically acceptable salt thereof, is at least 90% enantiomerically pure.

Also provided is N-methyl-4,5,6,7-tetrahydrobenzothiophen-5-amine

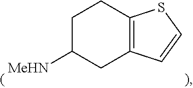 [SMILES: CNC1CCc2sccc2C1]

or pharmaceutically acceptable salt thereof, enriched in the (R) enantiomer. In some embodiments, the (R)—N-methyl-4,5,6,7-tetrahydrobenzothiophen-5-amine, or a pharmaceutically acceptable salt thereof, is at least 80% enantiomerically pure. In some embodiments, the (R)—N-methyl-4,5,6,7-tetrahydrobenzothiophen-5-amine, or a pharmaceutically acceptable salt thereof, is at least 85% enantiomerically pure. In some embodiments, the (R)—N-methyl-4,5,6,7-tetrahydrobenzothiophen-5-amine, or a pharmaceutically acceptable salt thereof, is at least 90% enantiomerically pure. In some embodiments, the (R)—N-methyl-4,5,6,7-tetrahydrobenzothiophen-5-amine, or a pharmaceutically acceptable salt thereof, is at least 95% enantiomerically pure. In some embodiments, the (R)—N-methyl-4,5,6,7-tetrahydrobenzothiophen-5-amine, or a pharmaceutically acceptable salt thereof, is at least 96% enantiomerically pure. In some embodiments, the (R)—N-methyl-4,5,6,7-tetrahydrobenzothiophen-5-amine, or a pharmaceutically acceptable salt thereof, is at least 97% enantiomerically pure. In some embodiments, the (R)—N-methyl-4,5,6,7-tetrahydrobenzothiophen-5-amine, or a pharmaceutically acceptable salt thereof, is at least 98% enantiomerically pure. In some embodiments, the (R)—N-methyl-4,5,6,7-tetrahydrobenzothiophen-5-amine, or a pharmaceutically acceptable salt thereof, is at least 99% enantiomerically pure. In some embodiments, the (R)—N-methyl-4,5,6,7-tetrahydrobenzothiophen-5-amine, or a pharmaceutically acceptable salt thereof, is at least 99.5% enantiomerically pure.

Also provided is 3-methyl-4,5,6,7-tetrahydrobenzothiophen-6-amine

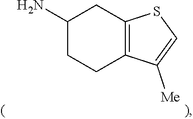 [SMILES: Cc1csc2c1CCC(N)C2]

or pharmaceutically acceptable salt thereof, enriched in the (R) enantiomer. In some embodiments, the (R)-3-methyl-4,5,6,7-tetrahydrobenzothiophen-6-amine, or a pharmaceutically acceptable salt thereof, is at least 80% enantiomerically pure. In some embodiments, the (R)-3-methyl-4,5,6,7-tetrahydrobenzothiophen-6-amine, or a pharmaceutically acceptable salt thereof, is at least 85% enantiomerically pure. In some embodiments, the (R)-3-methyl-4,5,6,7-tetrahydrobenzothiophen-6-amine, or a pharmaceutically acceptable salt thereof, is at least 90% enantiomerically pure. In some embodiments, the (R)-3-methyl-4,5,6,7-tetrahydrobenzothiophen-6-amine, or a pharmaceutically acceptable salt thereof, is at least 95% enantiomerically pure. In some embodiments, the (R)-3-methyl-4,5,6,7-tetrahydrobenzothiophen-6-amine, or a pharmaceutically acceptable salt thereof, is at least 96% enantiomerically pure. In some embodiments, the (R)-3-methyl-4,5,6,7-tetrahydrobenzothiophen-6-amine, or a pharmaceutically acceptable salt thereof, is at least 97% enantiomerically pure. In some embodiments, the (R)-3-methyl-4,5,6,7-tetrahydrobenzothiophen-6-amine, or a pharmaceutically acceptable salt thereof, is at least 98% enantiomerically pure. In some embodiments, the (R)-3-methyl-4,5,6,7-tetrahydrobenzothiophen-6-amine, or a pharmaceutically acceptable salt thereof, is at least 99% enantiomerically pure. In some embodiments, the (R)-3-methyl-4,5,6,7-tetrahydrobenzothiophen-6-amine, or a pharmaceutically acceptable salt thereof, is at least 99.5% enantiomerically pure.

Also provided is 2-chloro-N-methyl-4,5,6,7-tetrahydrobenzothiophen-5-amine

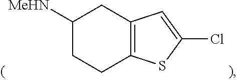 [SMILES: CNC1CCc2sc(Cl)cc2C1]

or a pharmaceutically acceptable salt thereof, enriched in the (R) enantiomer. In some embodiments, the (R)-2-chloro-N-methyl-4,5,6,7-tetrahydrobenzothiophen-5-amine, or a pharmaceutically acceptable salt thereof, is at least 80% enantiomerically pure. In some embodiments, the (R)-2-chloro-N-methyl-4,5,6,7-tetrahydrobenzothiophen-5-amine, or a pharmaceutically acceptable salt thereof, is at least 85% enantiomerically pure. In some embodiments, the (R)-2-chloro-N-methyl-4,5,6,7-tetrahydrobenzothiophen-5-amine, or a pharmaceutically acceptable salt thereof, is at least 90% enantiomerically pure. In some embodiments, the (R)-2-chloro-N-methyl-4,5,6,7-tetrahydrobenzothiophen-5-amine, or a pharmaceutically acceptable salt thereof, is at least 95% enantiomerically pure. In some embodiments, the (R)-2-chloro-N-methyl-4,5,6,7-tetrahydrobenzothiophen-5-amine, or a pharmaceutically acceptable salt thereof, is at least 96% enantiomerically pure. In some embodiments, the (R)-2-chloro-N-methyl-4,5,6,7-tetrahydrobenzothiophen-5-amine, or a pharmaceutically acceptable salt thereof, is at least 97% enantiomerically pure. In some embodiments, the (R)-2-chloro-N-methyl-4,5,6,7-tetrahydrobenzothiophen-5-amine, or a pharmaceutically acceptable salt thereof, is at least 98% enantiomerically pure. In some embodiments, the (R)-2-chloro-N-methyl-4,5,6,7-tetrahydrobenzothiophen-5-amine, or a pharmaceutically acceptable salt thereof, is at least 99% enantiomerically pure. In some embodiments, the (R)-2-chloro-N-methyl-4,5,6,7-tetrahydrobenzothiophen-5-amine, or a pharmaceutically acceptable salt thereof, is at least 99.5% enantiomerically pure.

Also provided is 4,5,6,7-tetrahydro-2-benzothiophen-5-amine

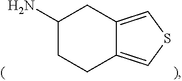

or a pharmaceutically acceptable salt thereof, enriched in the (R) enantiomer. In some embodiments, the (R)-4,5,6,7-tetrahydro-2-benzothiophen-5-amine, or a pharmaceutically acceptable salt thereof, is at least 80% enantiomerically pure. In some embodiments, the (R)-4,5,6,7-tetrahydro-2-benzothiophen-5-amine, or a pharmaceutically acceptable salt thereof, is at least 85% enantiomerically pure. In some embodiments, the (R)-4,5,6,7-tetrahydro-2-benzothiophen-5-amine, or a pharmaceutically acceptable salt thereof, is at least 90% enantiomerically pure. In some embodiments, the (R)-4,5,6,7-tetrahydro-2-benzothiophen-5-amine, or a pharmaceutically acceptable salt thereof, is at least 95% enantiomerically pure. In some embodiments, the (R)-4,5,6,7-tetrahydro-2-benzothiophen-5-amine, or a pharmaceutically acceptable salt thereof, is at least 96% enantiomerically pure. In some embodiments, the (R)-4,5,6,7-tetrahydro-2-benzothiophen-5-amine, or a pharmaceutically acceptable salt thereof, is at least 97% enantiomerically pure. In some embodiments, the (R)-4,5,6,7-tetrahydro-2-benzothiophen-5-amine, or a pharmaceutically acceptable salt thereof, is at least 98% enantiomerically pure. In some embodiments, the (R)-4,5,6,7-tetrahydro-2-benzothiophen-5-amine, or a pharmaceutically acceptable salt thereof, is at least 99% enantiomerically pure. In some embodiments, the (R)-4,5,6,7-tetrahydro-2-benzothiophen-5-amine, or a pharmaceutically acceptable salt thereof, is at least 99.5% enantiomerically pure.

Also provided is 4,5,6,7-tetrahydrobenzothiophen-5-amine

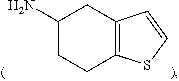

or a pharmaceutically acceptable salt thereof, enriched in the (R) enantiomer. In some embodiments, the (R)-4,5,6,7-tetrahydrobenzothiophen-5-amine, or a pharmaceutically acceptable salt thereof, is at least 80% enantiomerically pure. In some embodiments, the (R)-4,5,6,7-tetrahydrobenzothiophen-5-amine, or a pharmaceutically acceptable salt thereof, is at least 85% enantiomerically pure. In some embodiments, the (R)-4,5,6,7-tetrahydrobenzothiophen-5-amine, or a pharmaceutically acceptable salt thereof, is at least 90% enantiomerically pure. In some embodiments, the (R)-4,5,6,7-tetrahydrobenzothiophen-5-amine, or a pharmaceutically acceptable salt thereof, is at least 95% enantiomerically pure. In some embodiments, the (R)-4,5,6,7-tetrahydrobenzothiophen-5-amine, or a pharmaceutically acceptable salt thereof, is at least 96% enantiomerically pure. In some embodiments, the (R)-4,5,6,7-tetrahydrobenzothiophen-5-amine, or a pharmaceutically acceptable salt thereof, is at least 97% enantiomerically pure. In some embodiments, the (R)-4,5,6,7-tetrahydrobenzothiophen-5-amine, or a pharmaceutically acceptable salt thereof, is at least 98% enantiomerically pure. In some embodiments, the (R)-4,5,6,7-tetrahydrobenzothiophen-5-amine, or a pharmaceutically acceptable salt thereof, is at least 99% enantiomerically pure. In some embodiments, the (R)-4,5,6,7-tetrahydrobenzothiophen-5-amine, or a pharmaceutically acceptable salt thereof, is at least 99.5% enantiomerically pure.

Also provided is 2-chloro-4,5,6,7-tetrahydrobenzothiophen-5-amine

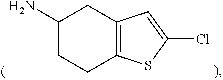 [SMILES: NC1CCc2sc(Cl)cc2C1]

or a pharmaceutically acceptable salt thereof, enriched in the (R) enantiomer. In some embodiments, the (R)-2-chloro-4,5,6,7-tetrahydrobenzothiophen-5-amine, or a pharmaceutically acceptable salt thereof, is at least 80% enantiomerically pure. In some embodiments, the (R)-2-chloro-4,5,6,7-tetrahydrobenzothiophen-5-amine, or a pharmaceutically acceptable salt thereof, is at least 85% enantiomerically pure. In some embodiments, the (R)-2-chloro-4,5,6,7-tetrahydrobenzothiophen-5-amine, or a pharmaceutically acceptable salt thereof, is at least 90% enantiomerically pure. In some embodiments, the (R)-2-chloro-4,5,6,7-tetrahydrobenzothiophen-5-amine, or a pharmaceutically acceptable salt thereof, is at least 95% enantiomerically pure. In some embodiments, the (R)-2-chloro-4,5,6,7-tetrahydrobenzothiophen-5-amine, or a pharmaceutically acceptable salt thereof, is at least 96% enantiomerically pure. In some embodiments, the (R)-2-chloro-4,5,6,7-tetrahydrobenzothiophen-5-amine, or a pharmaceutically acceptable salt thereof, is at least 97% enantiomerically pure. In some embodiments, the (R)-2-chloro-4,5,6,7-tetrahydrobenzothiophen-5-amine, or a pharmaceutically acceptable salt thereof, is at least 98% enantiomerically pure. In some embodiments, the (R)-2-chloro-4,5,6,7-tetrahydrobenzothiophen-5-amine, or a pharmaceutically acceptable salt thereof, is at least 99% enantiomerically pure. In some embodiments, the (R)-2-chloro-4,5,6,7-tetrahydrobenzothiophen-5-amine, or a pharmaceutically acceptable salt thereof, is at least 99.5% enantiomerically pure.

Also provided is 3-(trifluoromethyl)-4,5,6,7-tetrahydrobenzothiophen-6-amine

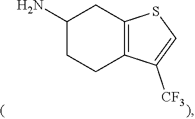 [SMILES: NC1CCc2c(C(F)(F)F)csc2C1]

or a pharmaceutically acceptable salt thereof, enriched in the (R) enantiomer. In some embodiments, the (R)-3-(trifluoromethyl)-4,5,6,7-tetrahydrobenzothiophen-6-amine, or a pharmaceutically acceptable salt thereof, is at least 80% enantiomerically pure. In some embodiments, the (R)-3-(trifluoromethyl)-4,5,6,7-tetrahydrobenzothiophen-6-amine, or a pharmaceutically acceptable salt thereof, is at least 85% enantiomerically pure. In some embodiments, the (R)-3-(trifluoromethyl)-4,5,6,7-tetrahydrobenzothiophen-6-amine, or a pharmaceutically acceptable salt thereof, is at least 90% enantiomerically pure. In some embodiments, the (R)-3-(trifluoromethyl)-4,5,6,7-tetrahydrobenzothiophen-6-amine, or a pharmaceutically acceptable salt thereof, is at least 95% enantiomerically pure. In some embodiments, the (R)-3-(trifluoromethyl)-4,5,6,7-tetrahydrobenzothiophen-6-amine, or a pharmaceutically acceptable salt thereof, is at least 96% enantiomerically pure. In some embodiments, the (R)-3-(trifluoromethyl)-4,5,6,7-tetrahydrobenzothiophen-6-amine, or a pharmaceutically acceptable salt thereof, is at least 97% enantiomerically pure. In some embodiments, the (R)-3-(trifluoromethyl)-4,5,6,7-tetrahydrobenzothiophen-6-amine, or a pharmaceutically acceptable salt thereof, is at least 98% enantiomerically pure. In some embodiments, the (R)-3-(trifluoromethyl)-4,5,6,7-tetrahydrobenzothiophen-6-amine, or a pharmaceutically acceptable salt thereof, is at least 99% enantiomerically pure. In some embodiments, the (R)-3-(trifluoromethyl)-4,5,6,7-tetrahydrobenzothiophen-6-amine, or a pharmaceutically acceptable salt thereof, is at least 99.5% enantiomerically pure.

Also provided is 5,6-dihydro-4H-cyclopenta[b]thiophen-5-amine

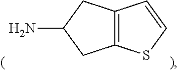

or a pharmaceutically acceptable salt thereof, enriched in the (R) enantiomer. In some embodiments, the (R)-5,6-dihydro-4H-cyclopenta[b]thiophen-5-amine, or a pharmaceutically acceptable salt thereof, is at least 80% enantiomerically pure. In some embodiments, the (R)-5,6-dihydro-4H-cyclopenta[b]thiophen-5-amine, or a pharmaceutically acceptable salt thereof, is at least 85% enantiomerically pure. In some embodiments, the (R)-5,6-dihydro-4H-cyclopenta[b]thiophen-5-amine, or a pharmaceutically acceptable salt thereof, is at least 90% enantiomerically pure. In some embodiments, the (R)-5,6-dihydro-4H-cyclopenta[b]thiophen-5-amine, or a pharmaceutically acceptable salt thereof, is at least 95% enantiomerically pure. In some embodiments, the (R)-5,6-dihydro-4H-cyclopenta[b]thiophen-5-amine, or a pharmaceutically acceptable salt thereof, is at least 96% enantiomerically pure. In some embodiments, the (R)-5,6-dihydro-4H-cyclopenta[b]thiophen-5-amine, or a pharmaceutically acceptable salt thereof, is at least 97% enantiomerically pure. In some embodiments, the (R)-5,6-dihydro-4H-cyclopenta[b]thiophen-5-amine, or a pharmaceutically acceptable salt thereof, is at least 98% enantiomerically pure. In some embodiments, the (R)-5,6-dihydro-4H-cyclopenta[b]thiophen-5-amine, or a pharmaceutically acceptable salt thereof, is at least 99% enantiomerically pure. In some embodiments, the (R)-5,6-dihydro-4H-cyclopenta[b]thiophen-5-amine, or a pharmaceutically acceptable salt thereof, is at least 99.5% enantiomerically pure.

Also provided is N-methyl-5,6,7,8-tetrahydro-4H-cyclohepta[b]thiophen-6-amine

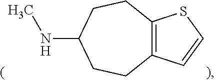

or a pharmaceutically acceptable salt thereof, enriched in the (R) enantiomer. In some embodiments, the (R)—N-methyl-5,6,7,8-tetrahydro-4H-cyclohepta[b]thiophen-6-amine, or a pharmaceutically acceptable salt thereof, is at least 80% enantiomerically pure. In some embodiments, the (R)—N-methyl-5,6,7,8-tetrahydro-4H-cyclohepta[b]thiophen-6-amine, or a pharmaceutically acceptable salt thereof, is at least 85% enantiomerically pure. In some embodiments, the (R)—N-methyl-5,6,7,8-tetrahydro-4H-cyclohepta[b]thiophen-6-amine, or a pharmaceutically acceptable salt thereof, is at least 90% enantiomerically pure. In some embodiments, the (R)—N-methyl-5,6,7,8-tetrahydro-4H-cyclohepta[b]thiophen-6-amine, or a pharmaceutically acceptable salt thereof, is at least 95% enantiomerically pure. In some embodiments, the (R)—N-methyl-5,6,7,8-tetrahydro-4H-cyclohepta[b]thiophen-6-amine, or a pharmaceutically acceptable salt thereof, is at least 96% enantiomerically pure. In some embodiments, the (R)—N-methyl-5,6,7,8-tetrahydro-4H-cyclohepta[b]thiophen-6-amine, or a pharmaceutically acceptable salt thereof, is at least 97% enantiomerically pure. In some embodiments, the (R)—N-methyl-5,6,7,8-tetrahydro-4H-cyclohepta[b]thiophen-6-amine, or a pharmaceutically acceptable salt thereof, is at least 98% enantiomerically pure. In some embodiments, the (R)—N-methyl-5,6,7,8-tetrahydro-4H-cyclohepta[b]thiophen-6-amine, or a pharmaceutically acceptable salt thereof, is at least 99% enantiomerically pure. In some embodiments, the (R)—N-methyl-5,6,7,8-tetrahydro-4H-cyclohepta[b]thiophen-6-amine, or a pharmaceutically acceptable salt thereof, is at least 99.5% enantiomerically pure.

Also provided is N-methyl-4,5,6,7-tetrahydrobenzothiophen-5-amine

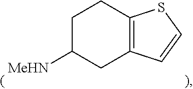 [SMILES: CNC1CCc2sccc2C1]

or pharmaceutically acceptable salt thereof, enriched in the (S) enantiomer. In some embodiments, the (S)—N-methyl-4,5,6,7-tetrahydrobenzothiophen-5-amine, or a pharmaceutically acceptable salt thereof, is at least 80% enantiomerically pure. In some embodiments, the (S)—N-methyl-4,5,6,7-tetrahydrobenzothiophen-5-amine, or a pharmaceutically acceptable salt thereof, is at least 85% enantiomerically pure. In some embodiments, the (S)—N-methyl-4,5,6,7-tetrahydrobenzothiophen-5-amine, or a pharmaceutically acceptable salt thereof, is at least 90% enantiomerically pure. In some embodiments, the (S)—N-methyl-4,5,6,7-tetrahydrobenzothiophen-5-amine, or a pharmaceutically acceptable salt thereof, is at least 95% enantiomerically pure. In some embodiments, the (S)—N-methyl-4,5,6,7-tetrahydrobenzothiophen-5-amine, or a pharmaceutically acceptable salt thereof, is at least 96% enantiomerically pure. In some embodiments, the (S)—N-methyl-4,5,6,7-tetrahydrobenzothiophen-5-amine, or a pharmaceutically acceptable salt thereof, is at least 97% enantiomerically pure. In some embodiments, the (S)—N-methyl-4,5,6,7-tetrahydrobenzothiophen-5-amine, or a pharmaceutically acceptable salt thereof, is at least 98% enantiomerically pure. In some embodiments, the (S)—N-methyl-4,5,6,7-tetrahydrobenzothiophen-5-amine, or a pharmaceutically acceptable salt thereof, is at least 99% enantiomerically pure. In some embodiments, the (S)—N-methyl-4,5,6,7-tetrahydrobenzothiophen-5-amine, or a pharmaceutically acceptable salt thereof, is at least 99.5% enantiomerically pure.

Also provided is 3-methyl-4,5,6,7-tetrahydrobenzothiophen-6-amine

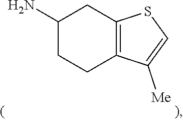 [SMILES: Cc1csc2c1CCC(N)C2]

or pharmaceutically acceptable salt thereof, enriched in the (S) enantiomer. In some embodiments, the (S)-3-methyl-4,5,6,7-tetrahydrobenzothiophen-6-amine, or a pharmaceutically acceptable salt thereof, is at least 80% enantiomerically pure. In some embodiments, the (S)-3-methyl-4,5,6,7-tetrahydrobenzothiophen-6-amine, or a pharmaceutically acceptable salt thereof, is at least 85% enantiomerically pure. In some embodiments, the (S)-3-methyl-4,5,6,7-tetrahydrobenzothiophen-6-amine, or a pharmaceutically acceptable salt thereof, is at least 90% enantiomerically pure. In some embodiments, the (S)-3-methyl-4,5,6,7-tetrahydrobenzothiophen-6-amine, or a pharmaceutically acceptable salt thereof, is at least 95% enantiomerically pure. In some embodiments, the (S)-3-methyl-4,5,6,7-tetrahydrobenzothiophen-6-amine, or a pharmaceutically acceptable salt thereof, is at least 96% enantiomerically pure. In some embodiments, the (S)-3-methyl-4,5,6,7-tetrahydrobenzothiophen-6-amine, or a pharmaceutically acceptable salt thereof, is at least 97% enantiomerically pure. In some embodiments, the (S)-3-methyl-4,5,6,7-tetrahydrobenzothiophen-6-amine, or a pharmaceutically acceptable salt thereof, is at least 98% enantiomerically pure. In some embodiments, the (S)-3-methyl-4,5,6,7-tetrahydrobenzothiophen-6-amine, or a pharmaceutically acceptable salt thereof, is at least 99% enantiomerically pure. In some embodiments, the (S)-3-methyl-4,5,6,7-tetrahydrobenzothiophen-6-amine, or a pharmaceutically acceptable salt thereof, is at least 99.5% enantiomerically pure.

Also provided is 2-chloro-N-methyl-4,5,6,7-tetrahydrobenzothiophen-5-amine

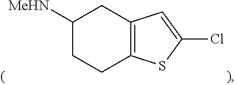 [SMILES: CNC1CCc2sc(Cl)cc2C1]

or a pharmaceutically acceptable salt thereof, enriched in the (S) enantiomer. In some embodiments, the (S)-2-chloro-N-methyl-4,5,6,7-tetrahydrobenzothiophen-5-amine, or a pharmaceutically acceptable salt thereof, is at least 80% enantiomerically pure. In some embodiments, the (S)-2-chloro-N-methyl-4,5,6,7-tetrahydrobenzothiophen-5-amine, or a pharmaceutically acceptable salt thereof, is at least 85% enantiomerically pure. In some embodiments, the (S)-2-chloro-N-methyl-4,5,6,7-tetrahydrobenzothiophen-5-amine, or a pharmaceutically acceptable salt thereof, is at least 90% enantiomerically pure. In some embodiments, the (S)-2-chloro-N-methyl-4,5,6,7-tetrahydrobenzothiophen-5-amine, or a pharmaceutically acceptable salt thereof, is at least 95% enantiomerically pure. In some embodiments, the (S)-2-chloro-N-methyl-4,5,6,7-tetrahydrobenzothiophen-5-amine, or a pharmaceutically acceptable salt thereof, is at least 96% enantiomerically pure. In some embodiments, the (S)-2-chloro-N-methyl-4,5,6,7-tetrahydrobenzothiophen-5-amine, or a pharmaceutically acceptable salt thereof, is at least 97% enantiomerically pure. In some embodiments, the (S)-2-chloro-N-methyl-4,5,6,7-tetrahydrobenzothiophen-5-amine, or a pharmaceutically acceptable salt thereof, is at least 98% enantiomerically pure. In some embodiments, the (S)-2-chloro-N-methyl-4,5,6,7-tetrahydrobenzothiophen-5-amine, or a pharmaceutically acceptable salt thereof, is at least 99% enantiomerically pure. In some embodiments, the (S)-2-chloro-N-methyl-4,5,6,7-tetrahydrobenzothiophen-5-amine, or a pharmaceutically acceptable salt thereof, is at least 99.5% enantiomerically pure.

Also provided is 4,5,6,7-tetrahydro-2-benzothiophen-5-amine

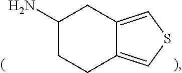

or a pharmaceutically acceptable salt thereof, enriched in the (S) enantiomer. In some embodiments, the (S)-4,5,6,7-tetrahydro-2-benzothiophen-5-amine, or a pharmaceutically acceptable salt thereof, is at least 80% enantiomerically pure. In some embodiments, the (S)-4,5,6,7-tetrahydro-2-benzothiophen-5-amine, or a pharmaceutically acceptable salt thereof, is at least 85% enantiomerically pure. In some embodiments, the (S)-4,5,6,7-tetrahydro-2-benzothiophen-5-amine, or a pharmaceutically acceptable salt thereof, is at least 90% enantiomerically pure. In some embodiments, the (S)-4,5,6,7-tetrahydro-2-benzothiophen-5-amine, or a pharmaceutically acceptable salt thereof, is at least 95% enantiomerically pure. In some embodiments, the (S)-4,5,6,7-tetrahydro-2-benzothiophen-5-amine, or a pharmaceutically acceptable salt thereof, is at least 96% enantiomerically pure. In some embodiments, the (S)-4,5,6,7-tetrahydro-2-benzothiophen-5-amine, or a pharmaceutically acceptable salt thereof, is at least 97% enantiomerically pure. In some embodiments, the (S)-4,5,6,7-tetrahydro-2-benzothiophen-5-amine, or a pharmaceutically acceptable salt thereof, is at least 98% enantiomerically pure. In some embodiments, the (S)-4,5,6,7-tetrahydro-2-benzothiophen-5-amine, or a pharmaceutically acceptable salt thereof, is at least 99% enantiomerically pure. In some embodiments, the (S)-4,5,6,7-tetrahydro-2-benzothiophen-5-amine, or a pharmaceutically acceptable salt thereof, is at least 99.5% enantiomerically pure.

Also provided is 4,5,6,7-tetrahydrobenzothiophen-5-amine

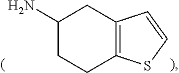

or a pharmaceutically acceptable salt thereof, enriched in the (S) enantiomer. In some embodiments, the (S)-4,5,6,7-tetrahydrobenzothiophen-5-amine, or a pharmaceutically acceptable salt thereof, is at least 80% enantiomerically pure. In some embodiments, the (S)-4,5,6,7-tetrahydrobenzothiophen-5-amine, or a pharmaceutically acceptable salt thereof, is at least 85% enantiomerically pure. In some embodiments, the (S)-4,5,6,7-tetrahydrobenzothiophen-5-amine, or a pharmaceutically acceptable salt thereof, is at least 90% enantiomerically pure. In some embodiments, the (S)-4,5,6,7-tetrahydrobenzothiophen-5-amine, or a pharmaceutically acceptable salt thereof, is at least 95% enantiomerically pure. In some embodiments, the (S)-4,5,6,7-tetrahydrobenzothiophen-5-amine, or a pharmaceutically acceptable salt thereof, is at least 96% enantiomerically pure. In some embodiments, the (S)-4,5,6,7-tetrahydrobenzothiophen-5-amine, or a pharmaceutically acceptable salt thereof, is at least 97% enantiomerically pure. In some embodiments, the (S)-4,5,6,7-tetrahydrobenzothiophen-5-amine, or a pharmaceutically acceptable salt thereof, is at least 98% enantiomerically pure. In some embodiments, the (S)-4,5,6,7-tetrahydrobenzothiophen-5-amine, or a pharmaceutically acceptable salt thereof, is at least 99% enantiomerically pure. In some embodiments, the (S)-4,5,6,7-tetrahydrobenzothiophen-5-amine, or a pharmaceutically acceptable salt thereof, is at least 99.5% enantiomerically pure.

Also provided is 2-chloro-4,5,6,7-tetrahydrobenzothiophen-5-amine

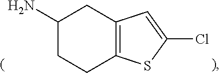 [SMILES: NC1CCc2sc(Cl)cc2C1]

or a pharmaceutically acceptable salt thereof, enriched in the (S) enantiomer. In some embodiments, the (S)-2-chloro-4,5,6,7-tetrahydrobenzothiophen-5-amine, or a pharmaceutically acceptable salt thereof, is at least 80% enantiomerically pure. In some embodiments, the (S)-2-chloro-4,5,6,7-tetrahydrobenzothiophen-5-amine, or a pharmaceutically acceptable salt thereof, is at least 85% enantiomerically pure. In some embodiments, the (S)-2-chloro-4,5,6,7-tetrahydrobenzothiophen-5-amine, or a pharmaceutically acceptable salt thereof, is at least 90% enantiomerically pure. In some embodiments, the (S)-2-chloro-4,5,6,7-tetrahydrobenzothiophen-5-amine, or a pharmaceutically acceptable salt thereof, is at least 95% enantiomerically pure. In some embodiments, the (S)-2-chloro-4,5,6,7-tetrahydrobenzothiophen-5-amine, or a pharmaceutically acceptable salt thereof, is at least 96% enantiomerically pure. In some embodiments, the (S)-2-chloro-4,5,6,7-tetrahydrobenzothiophen-5-amine, or a pharmaceutically acceptable salt thereof, is at least 97% enantiomerically pure. In some embodiments, the (S)-2-chloro-4,5,6,7-tetrahydrobenzothiophen-5-amine, or a pharmaceutically acceptable salt thereof, is at least 98% enantiomerically pure. In some embodiments, the (S)-2-chloro-4,5,6,7-tetrahydrobenzothiophen-5-amine, or a pharmaceutically acceptable salt thereof, is at least 99% enantiomerically pure. In some embodiments, the (S)-2-chloro-4,5,6,7-tetrahydrobenzothiophen-5-amine, or a pharmaceutically acceptable salt thereof, is at least 99.5% enantiomerically pure.

Also provided is 3-(trifluoromethyl)-4,5,6,7-tetrahydrobenzothiophen-6-amine

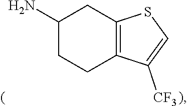 [SMILES: NC1CCc2c(C(F)(F)F)csc2C1]

or a pharmaceutically acceptable salt thereof, enriched in the (S) enantiomer. In some embodiments, the (S)-3-(trifluoromethyl)-4,5,6,7-tetrahydrobenzothiophen-6-amine, or a pharmaceutically acceptable salt thereof, is at least 80% enantiomerically pure. In some embodiments, the (S)-3-(trifluoromethyl)-4,5,6,7-tetrahydrobenzothiophen-6-amine, or a pharmaceutically acceptable salt thereof, is at least 85% enantiomerically pure. In some embodiments, the (S)-3-(trifluoromethyl)-4,5,6,7-tetrahydrobenzothiophen-6-amine, or a pharmaceutically acceptable salt thereof, is at least 90% enantiomerically pure. In some embodiments, the (S)-3-(trifluoromethyl)-4,5,6,7-tetrahydrobenzothiophen-6-amine, or a pharmaceutically acceptable salt thereof, is at least 95% enantiomerically pure. In some embodiments, the (S)-3-(trifluoromethyl)-4,5,6,7-tetrahydrobenzothiophen-6-amine, or a pharmaceutically acceptable salt thereof, is at least 96% enantiomerically pure. In some embodiments, the (S)-3-(trifluoromethyl)-4,5,6,7-tetrahydrobenzothiophen-6-amine, or a pharmaceutically acceptable salt thereof, is at least 97% enantiomerically pure. In some embodiments, the (S)-3-(trifluoromethyl)-4,5,6,7-tetrahydrobenzothiophen-6-amine, or a pharmaceutically acceptable salt thereof, is at least 98% enantiomerically pure. In some embodiments, the (S)-3-(trifluoromethyl)-4,5,6,7-tetrahydrobenzothiophen-6-amine, or a pharmaceutically acceptable salt thereof, is at least 99% enantiomerically pure. In some embodiments, the (S)-3-(trifluoromethyl)-4,5,6,7-tetrahydrobenzothiophen-6-amine, or a pharmaceutically acceptable salt thereof, is at least 99.5% enantiomerically pure.

Also provided is 5,6-dihydro-4H-cyclopenta[b]thiophen-5-amine

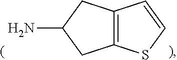

or a pharmaceutically acceptable salt thereof, enriched in the (S) enantiomer. In some embodiments, the (S)-5,6-dihydro-4H-cyclopenta[b]thiophen-5-amine, or a pharmaceutically acceptable salt thereof, is at least 80% enantiomerically pure. In some embodiments, the (S)-5,6-dihydro-4H-cyclopenta[b]thiophen-5-amine, or a pharmaceutically acceptable salt thereof, is at least 85% enantiomerically pure. In some embodiments, the (S)-5,6-dihydro-4H-cyclopenta[b]thiophen-5-amine, or a pharmaceutically acceptable salt thereof, is at least 90% enantiomerically pure. In some embodiments, the (S)-5,6-dihydro-4H-cyclopenta[b]thiophen-5-amine, or a pharmaceutically acceptable salt thereof, is at least 95% enantiomerically pure. In some embodiments, the (S)-5,6-dihydro-4H-cyclopenta[b]thiophen-5-amine, or a pharmaceutically acceptable salt thereof, is at least 96% enantiomerically pure. In some embodiments, the (S)-5,6-dihydro-4H-cyclopenta[b]thiophen-5-amine, or a pharmaceutically acceptable salt thereof, is at least 97% enantiomerically pure. In some embodiments, the (S)-5,6-dihydro-4H-cyclopenta[b]thiophen-5-amine, or a pharmaceutically acceptable salt thereof, is at least 98% enantiomerically pure. In some embodiments, the (S)-5,6-dihydro-4H-cyclopenta[b]thiophen-5-amine, or a pharmaceutically acceptable salt thereof, is at least 99% enantiomerically pure. In some embodiments, the (S)-5,6-dihydro-4H-cyclopenta[b]thiophen-5-amine, or a pharmaceutically acceptable salt thereof, is at least 99.5% enantiomerically pure.

Also provided is N-methyl-5,6,7,8-tetrahydro-4H-cyclohepta[b]thiophen-6-amine

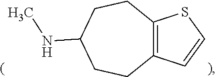

or a pharmaceutically acceptable salt thereof, enriched in the (S) enantiomer. In some embodiments, the (S)—N-methyl-5,6,7,8-tetrahydro-4H-cyclohepta[b]thiophen-6-amine, or a pharmaceutically acceptable salt thereof, is at least 80% enantiomerically pure. In some embodiments, the (S)—N-methyl-5,6,7,8-tetrahydro-4H-cyclohepta[b]thiophen-6-amine, or a pharmaceutically acceptable salt thereof, is at least 85% enantiomerically pure. In some embodiments, the (S)—N-methyl-5,6,7,8-tetrahydro-4H-cyclohepta[b]thiophen-6-amine, or a pharmaceutically acceptable salt thereof, is at least 90% enantiomerically pure. In some embodiments, the (S)—N-methyl-5,6,7,8-tetrahydro-4H-cyclohepta[b]thiophen-6-amine, or a pharmaceutically acceptable salt thereof, is at least 95% enantiomerically pure. In some embodiments, the (S)—N-methyl-5,6,7,8-tetrahydro-4H-cyclohepta[b]thiophen-6-amine, or a pharmaceutically acceptable salt thereof, is at least 96% enantiomerically pure. In some embodiments, the (S)—N-methyl-5,6,7,8-tetrahydro-4H-cyclohepta[b]thiophen-6-amine, or a pharmaceutically acceptable salt thereof, is at least 97% enantiomerically pure. In some embodiments, the (S)—N-methyl-5,6,7,8-tetrahydro-4H-cyclohepta[b]thiophen-6-amine, or a pharmaceutically acceptable salt thereof, is at least 98% enantiomerically pure. In some embodiments, the (S)—N-methyl-5,6,7,8-tetrahydro-4H-cyclohepta[b]thiophen-6-amine, or a pharmaceutically acceptable salt thereof, is at least 99% enantiomerically pure. In some embodiments, the (S)—N-methyl-5,6,7,8-tetrahydro-4H-cyclohepta[b]thiophen-6-amine, or a pharmaceutically acceptable salt thereof, is at least 99.5% enantiomerically pure.

In some embodiments, the compound of Formula I, Formula Ia, Formula Ib, Formula Ic, Formula Id, Formula Ie, Formula If, or Formula Ig, Formula Ih, or pharmaceutically acceptable salt thereof, is crystalline. In some embodiments, the crystalline compound, or pharmaceutically acceptable salt thereof, is crystalline N-methyl-4,5,6,7-tetrahydro-2-benzothiophen-5-amine hydrochloride. In some embodiments, the crystalline compound, or pharmaceutically acceptable salt thereof, is crystalline 3-methyl-4,5,6,7-tetrahydrobenzothiophen-6-amine hydrochloride. In some embodiments, the crystalline compound, or pharmaceutically acceptable salt thereof, is crystalline 2-chloro-N-methyl-4,5,6,7-tetrahydrobenzothiophen-5-amine hydrochloride. In some embodiments, the crystalline compound, or pharmaceutically acceptable salt thereof, is crystalline 4,5,6,7-tetrahydro-2-benzothiophen-5-amine hydrochloride. In some embodiments, the crystalline compound, or pharmaceutically acceptable salt thereof, is crystalline 4,5,6,7-tetrahydrobenzothiophen-5-amine hydrochloride. In some embodiments, the crystalline compound, or pharmaceutically acceptable salt thereof, is crystalline 2-chloro-4,5,6,7-tetrahydrobenzothiophen-5-amine hydrochloride. In some embodiments, the crystalline compound, or pharmaceutically acceptable salt thereof, is crystalline 3-(trifluoromethyl)-4,5,6,7-tetrahydrobenzothiophen-6-amine hydrochloride. In some embodiments, the crystalline compound, or pharmaceutically acceptable salt thereof, is crystalline 5,6-dihydro-4H-cyclopenta[b]thiophen-5-amine hydrochloride. In some embodiments, the crystalline compound, or pharmaceutically acceptable salt thereof, is crystalline N-methyl-5,6,7,8-tetrahydro-4H-cyclohepta[b]thiophen-6-amine hydrochloride.

Also provided is a pharmaceutically acceptable salt of a compound of Formula I, Formula Ia, Formula Ib, Formula Ic, Formula Id, Formula Ie, Formula If, or Formula Ig, Formula Ih. In some embodiments, the pharmaceutically acceptable salt is a hydrochloride salt. In some embodiments, the pharmaceutically acceptable salt is N-methyl-4,5,6,7-tetrahydro-2-benzothiophen-5-amine hydrochloride. In some embodiments, the pharmaceutically acceptable salt is 3-methyl-4,5,6,7-tetrahydrobenzothiophen-6-amine hydrochloride. In some embodiments, the pharmaceutically acceptable salt is 2-chloro-N-methyl-4,5,6,7-tetrahydrobenzothiophen-5-amine hydrochloride. In some embodiments, the pharmaceutically acceptable salt is 4,5,6,7-tetrahydro-2-benzothiophen-5-amine hydrochloride. In some embodiments, the pharmaceutically acceptable salt is 4,5,6,7-tetrahydrobenzothiophen-5-amine hydrochloride. In some embodiments, the pharmaceutically acceptable salt is 2-chloro-4,5,6,7-tetrahydrobenzothiophen-5-amine hydrochloride. In some embodiments, the pharmaceutically acceptable salt is 3-(trifluoromethyl)-4,5,6,7-tetrahydrobenzothiophen-6-amine hydrochloride. In some embodiments, the pharmaceutically acceptable salt is 5,6-dihydro-4H-cyclopenta[b]thiophen-5-amine hydrochloride. In some embodiments, the pharmaceutically acceptable salt is N-methyl-5,6,7,8-tetrahydro-4H-cyclohepta[b]thiophen-6-amine hydrochloride.

Also provided is a pharmaceutical composition comprising a compound or pharmaceutically acceptable salt described herein, together with at least one pharmaceutically acceptable excipient.

Also provided is a method of treating a CNS disease or disorder in a patient in need thereof, comprising administering to the patient a therapeutically effective amount of a compound or pharmaceutically acceptable salt thereof described herein, or a pharmaceutical composition described herein, wherein the CNS disease or disorder is depression, schizophrenia, or an anxiety disorder. In some embodiments, the patient is administered a therapeutically effective amount of N-methyl-4,5,6,7-tetrahydrobenzothiophen-5-amine

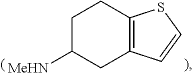 [SMILES: CNC1CCc2sccc2C1]

or a pharmaceutically acceptable salt thereof. In some embodiments, the patient is administered a therapeutically effective amount of 3-methyl-4,5,6,7-tetrahydrobenzothiophen-6-amine

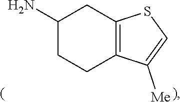 [SMILES: Cc1csc2c1CCC(N)C2]

or a pharmaceutically acceptable salt thereof. In some embodiments, the patient is administered a therapeutically effective amount of 2-chloro-N-methyl-4,5,6,7-tetrahydrobenzothiophen-5-amine

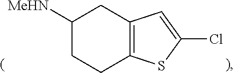 [SMILES: CNC1CCc2sc(Cl)cc2C1]

or a pharmaceutically acceptable salt thereof. In some embodiments, the patient is administered a therapeutically effective amount of 4,5,6,7-tetrahydro-2-benzothiophen-5-amine

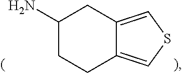

or a pharmaceutically acceptable salt thereof. In some embodiments, the patient is administered a therapeutically effective amount of 4,5,6,7-tetrahydrobenzothiophen-5-amine

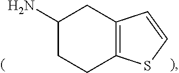

or a pharmaceutically acceptable salt thereof. In some embodiments, the patient is administered a therapeutically effective amount of 2-chloro-4,5,6,7-tetrahydrobenzothiophen-5-amine

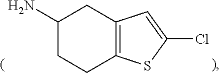 [SMILES: NC1CCc2sc(Cl)cc2C1]

or a pharmaceutically acceptable salt thereof. In some embodiments, the patient is administered a therapeutically effective amount of 3-(trifluoromethyl)-4,5,6,7-tetrahydrobenzothiophen-6-amine

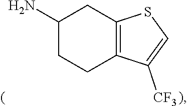 [SMILES: NC1CCc2c(C(F)(F)F)csc2C1]

or a pharmaceutically acceptable salt thereof. In some embodiments, the patient is administered a therapeutically effective amount of 5,6-dihydro-4H-cyclopenta[b]thiophen-5-amine

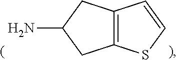

or a pharmaceutically acceptable salt thereof. In some embodiments, the patient is administered a therapeutically effective amount of N-methyl-5,6,7,8-tetrahydro-4H-cyclohepta[b]thiophen-6-amine

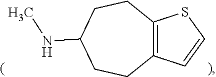

or a pharmaceutically acceptable salt thereof. In some embodiments, the N-methyl-4,5,6,7-tetrahydrobenzothiophen-5-amine, 3-methyl-4,5,6,7-tetrahydrobenzothiophen-6-amine, 2-chloro-N-methyl-4,5,6,7-tetrahydrobenzothiophen-5-amine, 4,5,6,7-tetrahydro-2-benzothiophen-5-amine, 4,5,6,7-tetrahydrobenzothiophen-5-amine, 2-chloro-4,5,6,7-tetrahydrobenzothiophen-5-amine, 3-(trifluoromethyl)-4,5,6,7-tetrahydrobenzothiophen-6-amine, 5,6-dihydro-4H-cyclopenta[b]thiophen-5-amine, N-methyl-5,6,7,8-tetrahydro-4H-cyclohepta[b]thiophen-6-amine, or pharmaceutically acceptable salt thereof, is enriched in the (R) enantiomer. In some embodiments, the N-methyl-4,5,6,7-tetrahydrobenzothiophen-5-amine, 3-methyl-4,5,6,7-tetrahydrobenzothiophen-6-amine, 2-chloro-N-methyl-4,5,6,7-tetrahydrobenzothiophen-5-amine, 4,5,6,7-tetrahydro-2-benzothiophen-5-amine, 4,5,6,7-tetrahydrobenzothiophen-5-amine, 2-chloro-4,5,6,7-tetrahydrobenzothiophen-5-amine, 3-(trifluoromethyl)-4,5,6,7-tetrahydrobenzothiophen-6-amine, 5,6-dihydro-4H-cyclopenta[b]thiophen-5-amine, N-methyl-5,6,7,8-tetrahydro-4H-cyclohepta[b]thiophen-6-amine, or pharmaceutically acceptable salt thereof, is enriched in the (S) enantiomer. In some embodiments, the (R)—N-methyl-4,5,6,7-tetrahydrobenzothiophen-5-amine, (S)—N-methyl-4,5,6,7-tetrahydrobenzothiophen-5-amine, (R)-3-methyl-4,5,6,7-tetrahydrobenzothiophen-6-amine, (S)-3-methyl-4,5,6,7-tetrahydrobenzothiophen-6-amine, (R)-2-chloro-N-methyl-4,5,6,7-tetrahydrobenzothiophen-5-amine, (S)-2-chloro-N-methyl-4,5,6,7-tetrahydrobenzothiophen-5-amine, (R)-4,5,6,7-tetrahydro-2-benzothiophen-5-amine, (S)-4,5,6,7-tetrahydro-2-benzothiophen-5-amine, (R)-4,5,6,7-tetrahydrobenzothiophen-5-amine, (S)-4,5,6,7-tetrahydrobenzothiophen-5-amine, (R)-2-chloro-4,5,6,7-tetrahydrobenzothiophen-5-amine, (S)-2-chloro-4,5,6,7-tetrahydrobenzothiophen-5-amine, (R)-3-(trifluoromethyl)-4,5,6,7-tetrahydrobenzothiophen-6-amine, (S)-3-(trifluoromethyl)-4,5,6,7-tetrahydrobenzothiophen-6-amine, (R)-5,6-dihydro-4H-cyclopenta[b]thiophen-5-amine, (S)-5,6-dihydro-4H-cyclopenta[b]thiophen-5-amine, (R)—N-methyl-5,6,7,8-tetrahydro-4H-cyclohepta[b]thiophen-6-amine, (S)—N-methyl-5,6,7,8-tetrahydro-4H-cyclohepta[b]thiophen-6-amine, or pharmaceutically acceptable salt thereof, has an enantiomeric purity as described above.

In some embodiments, the CNS disease or disorder is depression. Depressive disorders are often associated with depressed mood (sadness), poor concentration, insomnia, fatigue, appetite disturbances, excessive guilt and thoughts of suicide. In some embodiments, provided are methods of treating one or more symptoms of depression.

In some embodiments, the CNS disease or disorder is an anxiety disorder.

In some embodiments, the CNS disease or disorder is schizophrenia. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the subject suffers from a lack of normal experiences, such as anhedonia and lack of social interaction. The cognitive symptoms relate to cognitive impairment in schizophrenics, such as lack of sustained attention and deficits in decision making.

### Pharmaceutical Compositions

While it may be possible for the compounds and salts described herein to be administered to a patient as the raw chemical, they are preferably administered as a pharmaceutical composition. Accordingly, provided herein are pharmaceutical compositions which comprise a compound described herein, or a pharmaceutically acceptable salt thereof, together with one or more pharmaceutically acceptable excipients. Any of the well-known techniques and excipients may be used as suitable and as understood in the art (see, e.g., Remington: The Science and Practice of Pharmacy, 20th ed., Gennaro et al. Eds., Lippincott Williams and Wilkins, 2000).

In certain embodiments, the pharmaceutical composition contains about 0.1 mg to about 2000 mg of a compound, or pharmaceutically acceptable salt thereof, described herein. In certain embodiments, the pharmaceutical composition contains about 0.5 mg to about 1500 mg of a compound, or pharmaceutically acceptable salt thereof, described herein. In certain embodiments, the pharmaceutical composition contains about 1 mg to about 1000 mg of a compound, or pharmaceutically acceptable salt thereof, described herein. In certain embodiments, the pharmaceutical composition contains about 0.25 mg, 0.5 mg, 1 mg, 5 mg, 10 mg, 50 mg, 100 mg, 200 mg, 300 mg, 400 mg, 500 mg, or 1000 mg of a compound, or pharmaceutically acceptable salt thereof, described herein. In certain embodiments, the pharmaceutical composition contains about 2 mg, 25 mg, 75 mg, or 150 mg of a compound, or pharmaceutically acceptable salt thereof, described herein.

Compounds and pharmaceutically acceptable salts thereof described herein may be administered at a dose of from 0.01 to 300 mg/kg per day, such as 0.03 to 100 mg/kg per day. The dose range for adult humans is generally from 0.05 mg to 2 g/day, such as for example 0.1 mg to 1 g/day, 0.1 to 500 mg/day, or 0.5 to 400 mg/day. Dosage forms provided in discrete units may conveniently contain an amount of one or more compounds or pharmaceutically acceptable salts thereof described herein which is effective at such dosage or as a multiple of the same, for instance, units containing 0.1 mg to 500 mg, usually around 0.25 mg to 100 mg.

Compressed oral tablets may comprise the following ingredients:

| Ingredient | | mg/tablet | | | |
|---|---|---|---|---|---|
| 1. | Compound or pharmaceutically | 5 | 25 | 100 | 500 |
| | acceptable salt thereof described | | | | |
| | herein | | | | |
| 2. | Anhydrous Lactose Direct Tableting | 125 | 105 | 30 | 150 |
| 3. | Starch 1500 (partially pregelatinized | 6 | 6 | 6 | 60 |
| | maize starch) | | | | |
| 4. | Microcrystalline Cellulose | 30 | 30 | 30 | 450 |
| 5. | Magnesium Stearate | 1 | 1 | 1 | 1 |

| | | | | |
|---|---|---|---|---|
| Total | 167 | 167 | 167 | 831 |

To make the compressed oral tablets, first thoroughly mix ingredients 1-4 and granulate with purified water; then dry the granules, mill the dried granules to desired particle size, and finally add ingredient 5, mix thoroughly, and compress.

Powder filled oral capsules may comprise the following ingredients:

| Ingredient | | mg/capsule | | | |
|---|---|---|---|---|---|
| 1. | Compound or pharmaceutically | 5 | 25 | 100 | 500 |
| | acceptable salt thereof | | | | |
| | described herein | | | | |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |

| | | | | |
|---|---|---|---|---|
| Total | 200 | 200 | 300 | 600 |

To make the powder filled oral capsules, first thoroughly mix ingredients 1-3 and mill to desired particle size; then add ingredients 4 and 5, mix thoroughly, and fill into capsules (e.g., hard gelatin capsules).

Sterile injection pharmaceutical composition may comprise the following ingredients:

| | | mg/sterile injection |
|---|---|---|
| Ingredient | | composition |
| 1. | Compound or pharmaceutically acceptable | 3 |
| | salt thereof described herein | |
| 2. | PEG 400 | 150 |
| 3. | Acetic Acid | to pH 5 |
| 4. | Water for Injection | to 1 mL |

To make the sterile injection pharmaceutical composition, mix thoroughly mix ingredients 1 and 2 and a portion of ingredient 4 to dissolve ingredient 1; then adjust to pH 5 with ingredient 3 and add ingredient 4 to 1 mL total volume; and finally filter, fill into vials, and sterilize.

### EXAMPLES

### Synthetic Scheme for the preparation of N-methyl-4,5,6,7-tetrahydrobenzothiophen-6-amine hydrochloride enantiomer 1 (9) and enantiomer 2 (10)

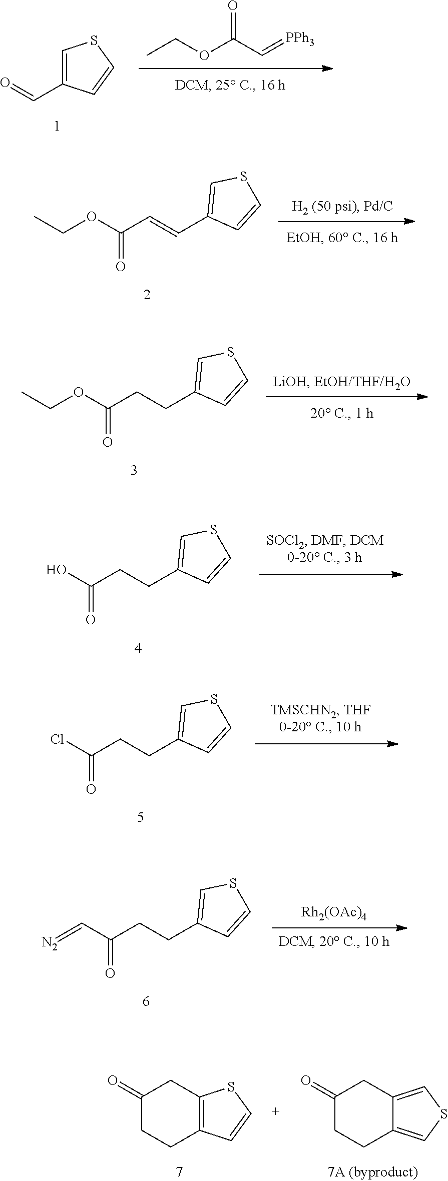

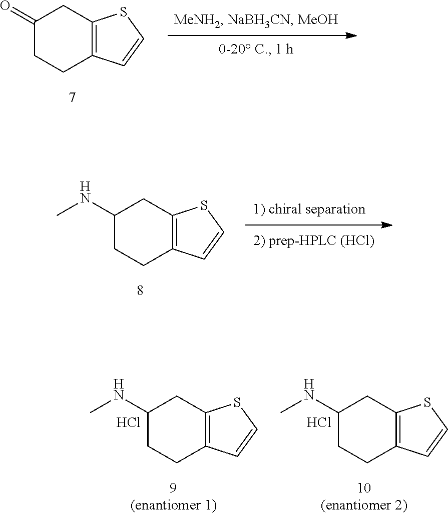

### Procedure for the preparation of ethyl (E)-3-(3-thienyl)prop-2-enoate (2)

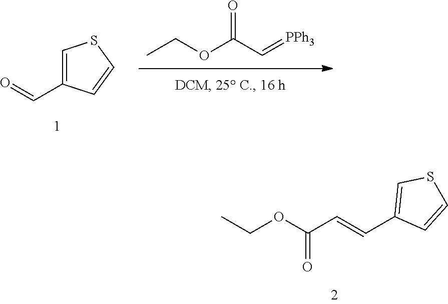

To a solution of thiophene-3-carbaldehyde (100 g, 892 mmol, 81.3 mL, 1 eq) in DCM (1.5 L) was added ethyl 2-(triphenyl-λ⁵-phosphanylidene)acetate (326 g, 936 mmol, 1.05 eq). The mixture was stirred at 25° C. for 16 hr. The reaction mixture was concentrated under reduced pressure. The residue was triturated with EtOAc (300 mL), and the precipitate (Ph₃P═O) was removed by filtration. The filtrate was concentrated under reduced pressure. The residue was purified by flash silica gel chromatography (eluting with 1:20 Ethyl acetate/Petroleum ether) to afford ethyl (E)-3-(3-thienyl)prop-2-enoate (2) (154 g, 845 mmol, 94.77% yield as a yellow oil. ¹H NMR (400 MHz, CHLOROFORM-d) δ 7.68 (d, J=15.9 Hz, 1H), 7.52-7.47 (m, 1H), 7.37-7.28 (m, 2H), 6.27 (d, J=15.9 Hz, 1H), 4.26 (q, J=7.1 Hz, 2H), 1.34 (t, J=7.1 Hz, 3H). LCMS: m/z [M+H]⁺=183.2.

### Procedure for the preparation of ethyl 3-(3-thienyl)propanoate (3)

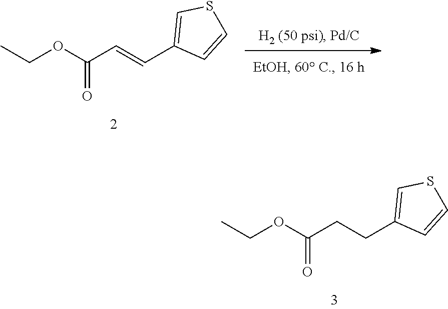

To a solution of ethyl (E)-3-(3-thienyl)prop-2-enoate (2) (150 g, 823 mmol, 1 eq) in EtOH (1.2 L) was added 10% Pd/C (40 g) under N₂. The suspension was degassed under vacuum and purged with H₂ several times. The mixture was stirred under H₂ (50 psi) at 60° C. for 16 hr. The reaction mixture was filtered, and the filtrate was concentrated to dryness to give ethyl 3-(3-thienyl)propanoate (3) (152 g, crude) as a yellow oil. ¹H NMR (400 MHz, CHLOROFORM-d) δ 7.27-7.24 (m, 1H), 7.00-6.97 (m, 1H), 6.97-6.94 (m, 1H), 4.18-4.09 (m, 2H), 2.98 (t, J=7.6 Hz, 2H), 2.63 (t, J=7.7 Hz, 2H), 1.25 (t, J=7.2 Hz, 3H).

### Procedure for the preparation of 3-(3-thienyl)propanoic acid (4)

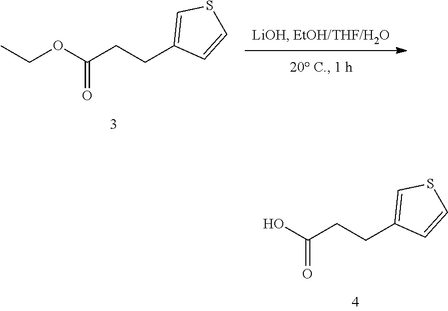

To a solution of ethyl 3-(3-thienyl)propanoate (3) (152 g, 825 mmol, crude, 1 eq) in THF (800 mL), H₂O (400 mL) and EtOH (50 mL) was added LiOH·H₂O (51.9 g, 1.24 μmol, 1.5 eq). The mixture was stirred at 20° C. for 1 hr. The reaction mixture was concentrated under reduced pressure to remove THF and EtOH, then diluted with H₂O (400 mL). The resulting mixture was treated with conc. HCl to pH 6. The precipitate was collected by filtration, washed with H₂O, and then dried under reduced pressure to afford 3-(3-thienyl)propanoic acid (4) (122 g, 94.9% over 2 steps) as a white solid. ¹H NMR (400 MHz, CHLOROFORM-d) δ 7.29 (dd, J=3.1, 4.8 Hz, 1H), 7.03 (d, J=1.6 Hz, 1H), 6.99 (d, J=4.9 Hz, 1H), 3.01 (t, J=7.6 Hz, 2H), 2.80-2.66 (m, 2H).

### Procedure for the preparation of 3-(3-thienyl)propanoyl chloride (5)

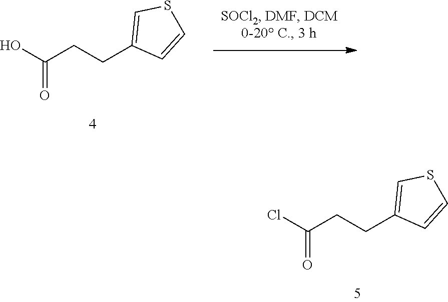 [SMILES: O=C(O)CCc1ccsc1 ; O=C(Cl)CCc1ccsc1]

To a solution of 3-(3-thienyl)propanoic acid (4) (109 g, 698 mmol, 1 eq) and DMF (941 mg, 12.9 mmol, 991 uL) in DCM (1 L) was added SOCl₂ (124.5 g, 1.05 μmol, 75.9 mL, 1.5 eq) slowly at 0° C. The mixture was stirred at 20° C. for 3 hr, and then concentrated under reduced pressure to give 3-(3-thienyl)propanoyl chloride (5) (120 g, crude) as a yellow oil.

### Procedure for the preparation of 1-diazo-4-(3-thienyl)butan-2-one (6) and 1-diazo-4-(3-thienyl)-1-trimethylsilyl-butan-2-one (6A)

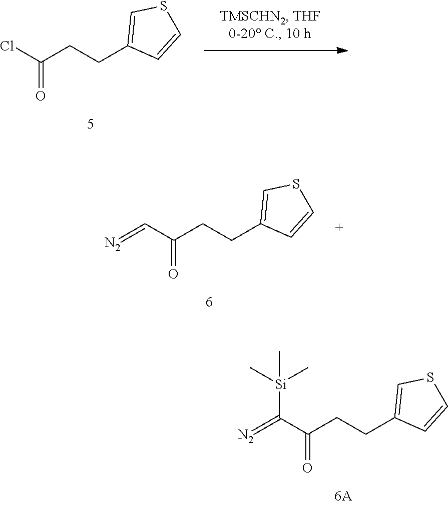

To a solution of 3-(3-thienyl)propanoyl chloride (5) (120 g, crude, 1 eq) in THF (200 mL) was added to TMSCHN₂ (2 M in hexane, 1.03 μL, 3 eq) dropwise at 0° C. The mixture was stirred at 20° C. for 10 hr. The reaction mixture was concentrated under reduced pressure. The residue was purified by flash silica gel chromatography (eluting with 1:50 Ethyl acetate/Petroleum ether) to afford 1-diazo-4-(3-thienyl)butan-2-one (6) (81.4 g, 451.65 mmol, 65.73% yield) as a yellow oil and 1-diazo-4-(3-thienyl)-1-trimethylsilyl-butan-2-one (6A) (36.9 g, 146.19 mmol, 21.28% yield) a yellow oil.

1-diazo-4-(3-thienyl)butan-2-one (6): ¹H NMR (400 MHz, CHLOROFORM-d) δ 7.30-7.25 (m, 1H), 7.00 (d, J=1.7 Hz, 1H), 6.96 (dd, J=1.2, 5.0 Hz, 1H), 5.24 (br s, 1H), 3.00 (t, J=7.6 Hz, 2H), 2.65 (br s, 2H).

1-diazo-4-(3-thienyl)-1-trimethylsilyl-butan-2-one (6A): ¹H NMR (400 MHz, CHLOROFORM-d) δ 7.26-7.24 (m, 1H), 6.98-6.93 (m, 2H), 3.03-2.98 (m, 2H), 2.85-2.78 (m, 2H), 0.27 (s, 9H).

### Procedure for the preparation of 1-diazo-4-(3-thienyl)butan-2-one (6)

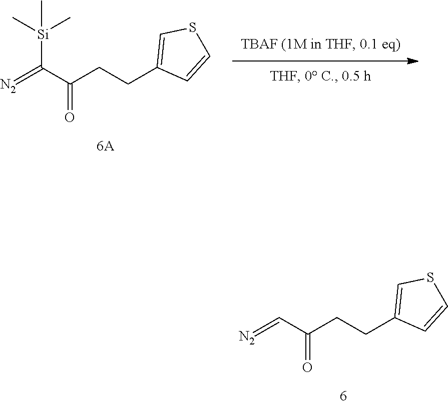

To a solution of 1-diazo-4-(3-thienyl)-1-trimethylsilyl-butan-2-one (6A) (41.0 g, 162 mmol, 1 eq) in THF (40 mL) was added TBAF (1.0 M in THF, 16.2 mL, 16.2 mmol, 0.1 eq) at 0° C. dropwise. The mixture was stirred at 0° C. for 0.5 hr. The reaction mixture was concentrated under reduced pressure. The residue was purified by flash silica gel chromatography (eluting with 1:5 Ethyl acetate/Petroleum ether) to afford 1-diazo-4-(3-thienyl)butan-2-one (6) (20 g, 111 mmol, 68.32% yield) as a yellow oil.

### Procedure for the preparation of 5,7-dihydro-4H-benzothiophen-6-one (7) and 6,7-dihydro-4H-2-benzothiophen-5-one (7A)

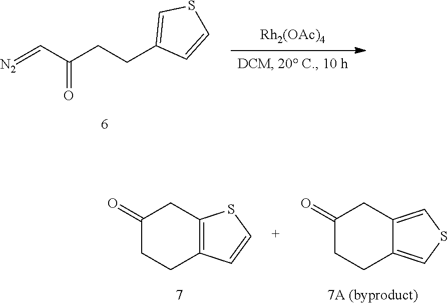

To a solution of 1-diazo-4-(3-thienyl)butan-2-one (100 g, 555 mmol, 277 mL, 1 eq) in DCM (4 L) was added Rh₂(OAc)₄ (200 mg, 453 μmol, 8.16 e-4 eq) under N₂. The mixture was stirred at 20° C. for 20 hr under N₂. The reaction mixture was concentrated under reduced pressure. The residue was purified by flash silica gel chromatography (eluting with 1:50 Ethyl acetate/Petroleum ether) to afford 5,7-dihydro-4H-benzothiophen-6-one (7) (37 g, 233 mmol, 42.06% yield) as a yellow oil and 6,7-dihydro-4H-2-benzothiophen-5-one (7A) (13 g, 78.57 mmol, 14.16% yield) as a yellow oil.

5,7-dihydro-4H-benzothiophen-6-one (7): 1H NMR (400 MHz, CHLOROFORM-d) δ 7.18 (d, J=5.1 Hz, 1H), 6.85 (d, J=5.0 Hz, 1H), 3.64 (s, 2H), 3.02 (t, J=6.8 Hz, 2H), 2.68 (t, J=6.8 Hz, 2H). LCMS: m/z [M+H]⁺=153.6.

6,7-dihydro-4H-2-benzothiophen-5-one (7A): 1H NMR (400 MHz, CHLOROFORM-d) δ 7.03 (d, J=2.8 Hz, 1H), 6.99 (d, J=14.2 Hz, 1H), 3.59 (s, 2H), 3.03 (t, J=6.7 Hz, 2H), 2.64-2.53 (m, 2H). LCMS: m/z [M+H]⁺=153.6.

### Procedure for the preparation of N-methyl-4,5,6,7-tetrahydrobenzothiophen-6-amine hydrochloride enantiomer 1 (9) and enantiomer 2 (10)

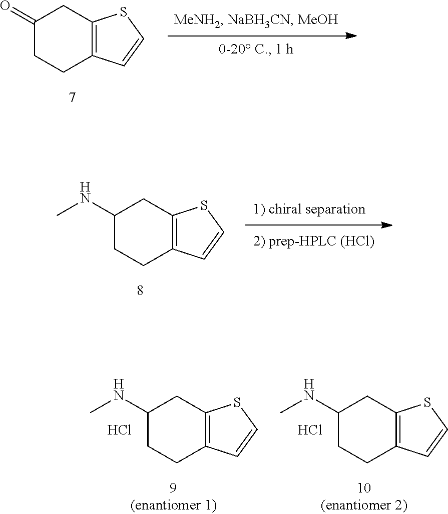

To a mixture of 5, 7-dihydro-4H-benzothiophen-6-one (7) (1.30 g, 8.54 mmol, 1 eq) and methylamine hydrochloride (2.88 g, 42.7 mmol, 5 eq) in MeOH (20 mL) was added NaBH₃CN (1.07 g, 17.1 mmol, 2 eq) at 0° C., the mixture was stirred at 20° C. for 1 hr. The reaction mixture was quenched by pouring into H₂O (10 mL). MeOH was removed through concentration under reduced pressure, and the resulting solution was extracted with EtOAc (10 mL*3). The combined organic extracts were dried over Na₂SO₄, filtered and concentrated under reduced pressure. The crude product was purified by reversed-phase column chromatography (C18 silica gel, 0.1% NH₃·H₂O) to give the racemate (8), which was separated by prep-Chiral-SFC (column: Phenomenex-Cellulose-2 (250 mm*30 mm, 10 m); Mobile phase A: Supercritical CO₂; Mobile phase B: 0.1% NH₃H₂O in MeOH; Gradient: isocratic 30% B; 4.9 min). The two enantiomers were separately further purified by prep-HPLC (column: 3_Phenomenex Luna C18 75*30 mm*3 μm; mobile phase: [water (HCl)-ACN]; B %: 1%-20%, 6 min) to give N-methyl-4, 5, 6, 7-tetrahydrobenzothiophen-6-amine hydrochloride salt (9) (81.46 mg, 399.84 μmol, 4.68% yield) as a yellow solid and N-methyl-4, 5, 6, 7-tetrahydrobenzothiophen-6-amine hydrochloride salt (10) (169.91 mg, 819.06 μmol, 9.59% yield) as an off-white solid.

Enantiomer 1 (9): ¹H NMR (400 MHz, METHANOL-d₄) δ 7.23 (d, J=5.1 Hz, 1H), 6.80 (d, J=5.1 Hz, 1H), 3.61-3.52 (m, 1H), 3.36-3.32 (m, 1H), 2.94-2.72 (m, 6H), 2.32-2.24 (m, 1H), 2.01-1.88 (m, 1H). LCMS: m/z [M+H]⁺=168.1.

Enantiomer 2 (10): ¹H NMR (400 MHz, METHANOL-d₄) δ 7.23 (d, J=5.1 Hz, 1H), 6.80 (d, J=5.1 Hz, 1H), 3.56 (dddd, J=2.9, 5.4, 8.2, 10.8 Hz, 1H), 3.35-3.32 (m, 1H), 2.94-2.73 (m, 6H), 2.33-2.23 (m, 1H), 2.01-1.89 (m, 1H). LCMS: m/z [M+H]⁺=168.1.

Synthetic Scheme for the preparation of N-methyl-4,5,6,7-tetrahydrobenzothiophen-5-amine hydrochloride enantiomer 1 (11) and enantiomer 2 (12)

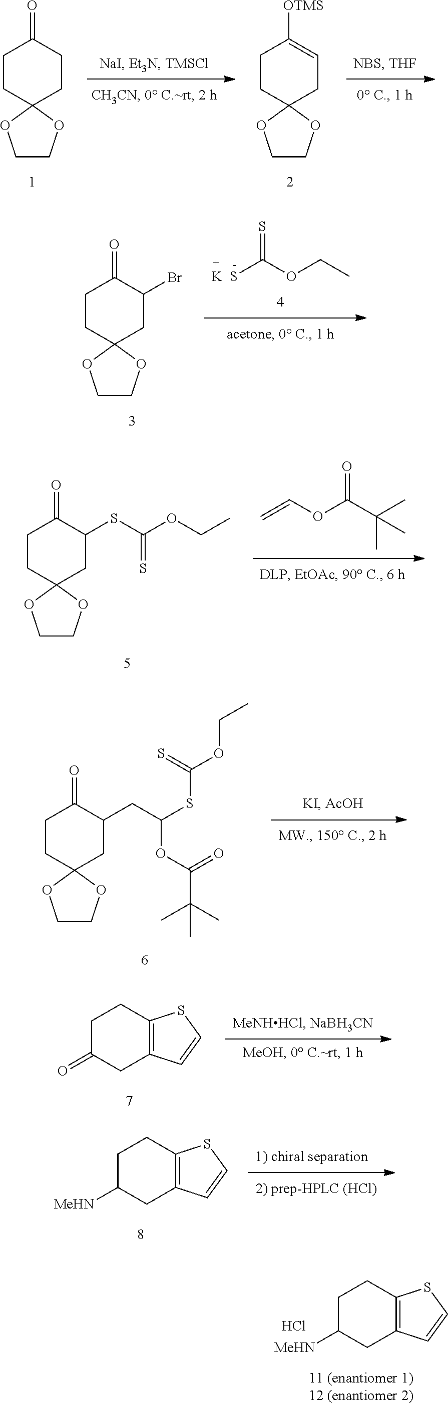

### Procedure for the preparation of 1,4-dioxaspiro[4.5]dec-7-en-8-yloxy(trimethyl)silane (2)

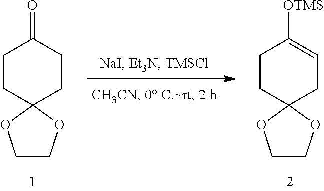

A solution of 1,4-dioxaspiro[4.5]decan-8-one (1) (5.0 g, 32.0 mmol, 1 eq) and NaI (5.76 g, 38.4 mmol, 1.2 eq) in CH₃CN (40 mL) was stirred at 20° C. for 10 min, then Et₃N (3.89 g, 38.4 mmol, 5.35 mL, 1.2 eq) and TMSCl (4.17 g, 38.4 mmol, 4.88 mL, 1.2 eq) were added at 0° C. The resulting mixture was stirred at 20° C. for 2 hr. The reaction mixture was quenched by adding sat. NaHCO₃ (50 mL), followed by extracting with EtOAc (40 mL*2). The combined organic layers were washed with brine (50 mL), dried over anhydrous Na₂SO₄, filtered, and concentrated under reduced pressure. The residue was purified by flash silica gel chromatography (eluting with 1:10 Ethyl acetate/Petroleum ether) to afford 1,4-dioxaspiro[4.5]dec-7-en-8-yloxy(trimethyl)silane (2) (6.8 g, 29.78 mmol, 93.01% yield) as a colorless liquid.

### Procedure for the preparation of 7-bromo-1,4-dioxaspiro[4.5]decan-8-one (3)

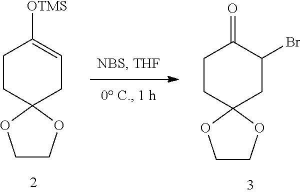

To a solution of 1,4-dioxaspiro[4.5]dec-7-en-8-yloxy(trimethyl)silane (2) (3.50 g, 15.3 mmol, 1 eq) in THF (30 mL) was added NBS (3.00 g, 16.9 mmol, 1.1 eq) batch-wise at 0° C. The resulting mixture was stirred at 0° C. for 1 hr. The reaction mixture was quenched by sat. NaHCO₃ (30 mL), followed by extracting with EtOAc (30 mL*2). The combined organic layers were washed with brine (30 mL), dried over anhydrous Na₂SO₄, filtered, and concentrated under reduced pressure to afford 7-bromo-1,4-dioxaspiro[4.5]decan-8-one (3) (3.6 g, crude) as a yellow oil.

### Procedure for the preparation of O-ethyl (8-oxo-1,4-dioxaspiro[4.5]decan-7-yl)sulfanyl methanethioate (5)

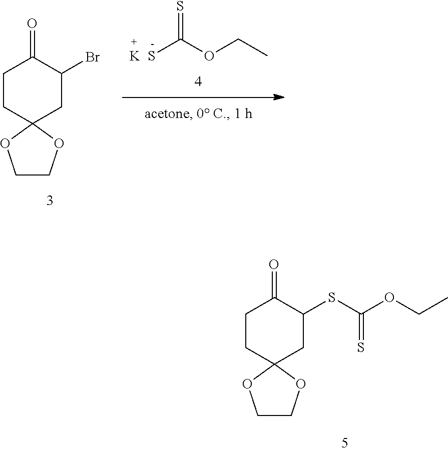

To a solution of 7-bromo-1,4-dioxaspiro[4.5]decan-8-one (3) (3.6 g, crude, 1 eq) in acetone (30 mL) was added potassium O-ethyl carbonodithioate (4) (2.5 g, 15.6 mmol, 1.02 eq) slowly at 0° C. The resulting mixture was stirred at 0° C. for 1 hr. The reaction mixture was filtered, and the filtrate was concentrated under reduced pressure. The residue was purified by flash silica gel chromatography (eluting with 1:5 Ethyl acetate/Petroleum ether) to afford O-ethyl (8-oxo-1,4-dioxaspiro[4.5]decan-7-yl)sulfanylmethanethioate (5) (3.3 g, 11.94 mmol, 77.97% yield) as a yellow oil. ¹H NMR (400 MHz, CHLOROFORM-d) δ 4.82 (ddd, J=0.7, 6.0, 13.1 Hz, 1H), 4.70-4.56 (m, 2H), 4.19-4.01 (m, 4H), 2.87-2.75 (m, 1H), 2.66-2.50 (m, 2H), 2.18-2.05 (m, 3H), 1.41 (t, J=7.1 Hz, 3H). LCMS: m/z [M+H]⁺=277.4.

### Procedure for the preparation of [1-ethoxycarbothioylsulfanyl-2-(8-oxo-1,4-dioxaspiro [4.5]decan-7-yl)ethyl]2,2-dimethylpropanoate (6)

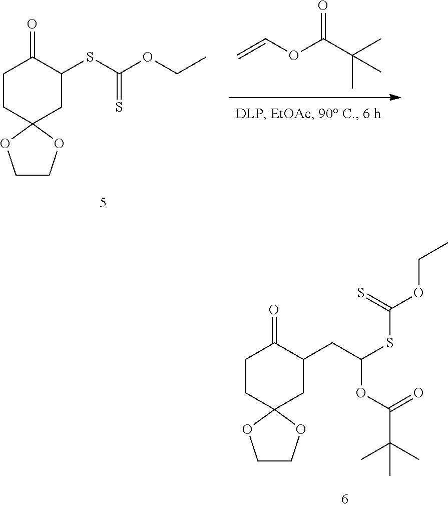

To a solution of O-ethyl (8-oxo-1,4-dioxaspiro[4.5]decan-7-yl)sulfanylmethanethioate (5) (3.3 g, 11.9 mmol, 1 eq) and vinyl 2,2-dimethylpropanoate (3.06 g, 23.9 mmol, 3.53 mL, 2 eq) in EtOAc (30 mL) was added lauroylperoxide (DLP) (476 mg, 1.19 mmol, 0.1 eq) under N₂. The mixture was stirred at 90° C. for 2 hr under N₂ before the addition of another batch of DLP (476 mg, 1.19 mmol, 0.1 eq), and the stirring was continued at 90° C. for another 4 hr. The reaction mixture was concentrated under reduced pressure. The residue was purified by flash silica gel chromatography (eluting with 1:5 Ethyl acetate/Petroleum ether) to afford [1-ethoxycarbothioylsulfanyl-2-(8-oxo-1,4-dioxaspiro[4.5]decan-7-yl)ethyl]2,2-dimethylpropanoate (6) (4.2 g, 7.79 mmol, 65.21% yield) as a yellow oil. ¹H NMR (400 MHz, CHLOROFORM-d) δ 6.70-6.62 (m, 1H), 4.68-4.58 (m, 2H), 4.11-3.97 (m, 4H), 2.84-2.49 (m, 3H), 2.44-2.19 (m, 2H), 2.08-1.92 (m, 2H), 1.82-1.66 (m, 2H), 1.46-1.39 (m, 3H), 1.20 (d, J=5.0 Hz, 9H).

### Procedure for the preparation of 6,7-dihydro-4H-benzothiophen-5-one (7)

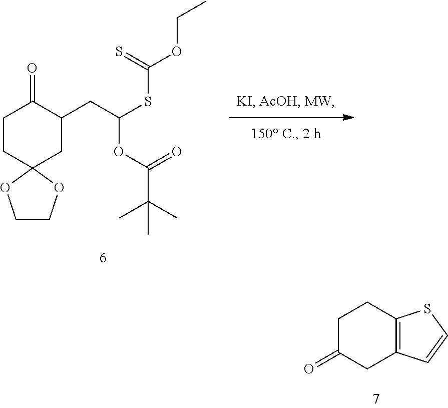 [SMILES: CCOC(=S)SC(CC1CC2(CCC1=O)OCCO2)OC(=O)C(C)(C)C ; "KI, AcOH, MW," ; O=C1CCc2sccc2C1]

A mixture of [1-ethoxycarbothioylsulfanyl-2-(8-oxo-1,4-dioxaspiro[4.5]decan-7-yl)ethyl]2,2-dimethylpropanoate (6) (4.0 g, 7.42 mmol, 1 eq), KI (2.46 g, 14.8 mmol, 2 eq) and AcOH (10 mL) was stirred at 150° C. for 2 hr under microwave irradiation. The reaction mixture was cooled to rt and concentrated under reduced pressure. The residue was treated with sat. aq. NaHCO₃ (10 mL) and extracted with EtOAc (10 mL*2). The combined organic layers were washed with brine (10 mL), dried over anhydrous Na₂SO₄, filtered, and concentrated under reduced pressure. The residue was purified by flash silica gel chromatography (eluting with 1:10 Ethyl acetate/Petroleum ether) to afford 6,7-dihydro-4H-benzothiophen-5-one (7) (1.0 g, 6.57 mmol, 88.59% yield) as a yellow oil. ¹H NMR (400 MHz, CHLOROFORM-d) δ 7.17 (d, J=5.1 Hz, 1H), 6.78 (d, J=5.0 Hz, 1H), 3.51 (s, 2H), 3.16 (t, J=6.8 Hz, 2H), 2.72 (t, J=6.8 Hz, 2H). LCMS: m/z [M+H]⁺=153.6.

### Procedure for the preparation of N-methyl-4,5,6,7-tetrahydrobenzothiophen-5-amine enantiomer hydrochloride enantiomer 1 (11) and enantiomer 2 (12)

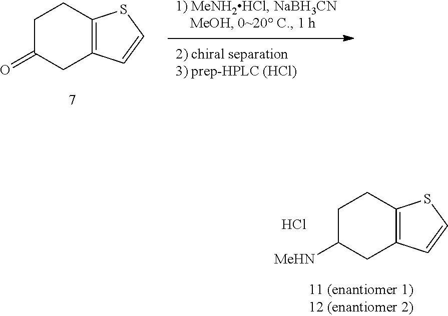

To a solution of 6,7-dihydro-4H-benzothiophen-5-one (7) (950 mg, 6.24 mmol, 1 eq) in MeOH (10 mL) was added methylamine hydrochloride (1.26 g, 18.7 mmol, 3 eq, HCl). The resulting mixture was stirred at 20° C. for 10 min before being treated with NaBH₃CN (1.96 g, 31.2 mmol, 5 eq) at 0° C. The reaction mixture was stirred at 0° C. for 1 hr. The reaction mixture was quenched with sat. NaHCO₃ (30 mL) and extracted with EtOAc (30 mL*3). The combined organic layers were washed with brine, dried over anhydrous Na₂SO₄, filtered, and concentrated under reduced pressure. The crude product was purified by flash silica gel chromatography (eluting with Ethyl acetate). The racemic product was subjected to prep-Chiral-SFC (Column: Phenomenex-Cellulose-2 (250 mm*30 mm, 10 μm); Mobile phase A: supercritical CO₂; Mobile phase B: 0.1% NH₃H₂O in MeOH; Gradient: isocratic 40% B, 2.5 min). The two enantiomers were separately further purified by prep-HPLC (HCl condition) to afford enantiomer 1 (11) (61 mg, 296.66 μmol, 4.75% yield; SFC purity=99.4% ee) and enantiomer 2 (12) (74 mg, 367.30 μmol, 5.88% yield; SFC purity=99.2% ee), both as white solids.

Enantiomer 1 (11): ¹H NMR (400 MHz, METHANOL-d4) δ 7.22 (d, J=5.1 Hz, 1H), 6.80 (d, J=5.1 Hz, 1H), 3.55-3.45 (m, 1H), 3.18 (dd, J=5.1, 15.8 Hz, 1H), 3.03-2.90 (m, 2H), 2.79 (s, 3H), 2.72 (dd, J=9.1, 15.7 Hz, 1H), 2.40-2.29 (m, 1H), 2.08-1.90 (m, 1H). LCMS: m/z [M+H]⁺=168.1.

Enantiomer 2 (12): ¹H NMR (400 MHz, METHANOL-d4) δ 7.22 (d, J=5.3 Hz, 1H), 6.81 (d, J=5.3 Hz, 1H), 3.57-3.46 (m, 1H), 3.19 (dd, J=5.0, 15.5 Hz, 1H), 3.05-2.88 (m, 2H), 2.79 (s, 3H), 2.72 (dd, J=9.2, 15.7 Hz, 1H), 2.38-2.29 (m, 1H), 2.04-1.92 (m, 1H). LCMS: m/z [M+H]⁺=168.1.

### Alternative synthetic scheme for preparation of N-methyl-4,5,6,7-tetrahydrobenzothiophen-5-amine enantiomer hydrochloride enantiomer 1 (11)

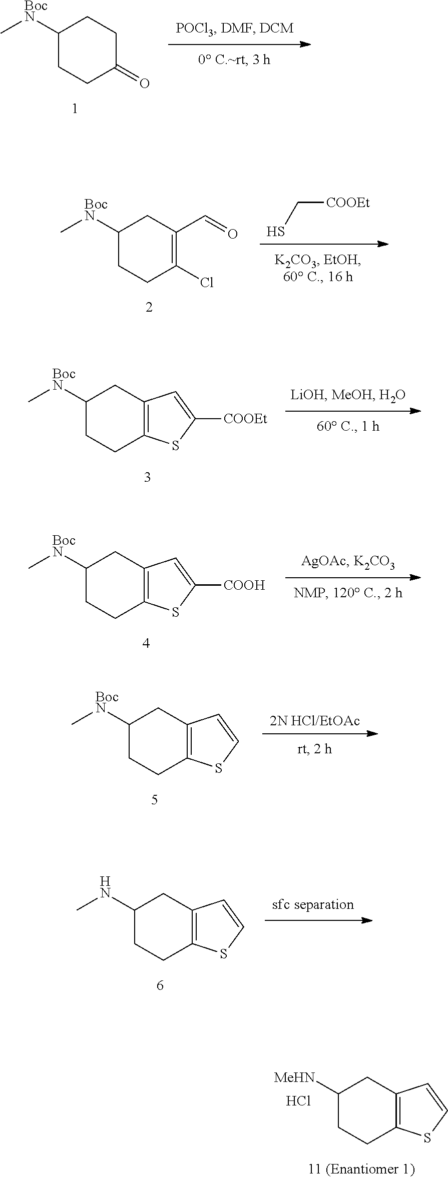

### Procedure for preparation of tert-butyl N-(4-chloro-3-formyl-cyclohex-3-en-1-yl)-N-methyl-carbamate (2)

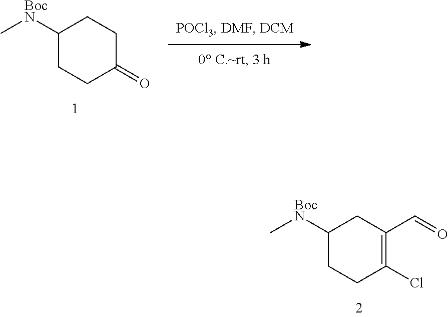

To a solution of DMF (48.2 g, 659 mmol, 50.7 mL, 6 eq) in DCM (300 mL) was added POCl₃ (84.3 g, 549.9 mmol, 51.2 mL, 5 eq) at 0° C. The mixture was stirred at 20° C. for 1 h, then was added tert-butyl N-methyl-N-(4-oxocyclohexyl)carbamate (1) (25 g, 109.99 mmol, 1 eq) in DCM (100 mL). The mixture was stirred at 0° C. for 2 h. The reaction mixture was quenched by addition of aq. sat. NaHCO₃ 1000 mL at 0° C., and extracted with EtOAc (500 mL*2). The combined organic layers were washed with aq. sat. NaCl (500 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~15% Ethyl acetate/Petroleum ether gradient @80 mL/min, short column). Compound tert-butyl N-(4-chloro-3-formyl-cyclohex-3-en-1-yl)-N-methyl-carbamate (2) (25 g, 91.32 mmol, 83.03% yield) was obtained as a white solid. ¹H NMR (400 MHz, CHLOROFORM-d) δ 10.13 (d, J=1.5 Hz, 1H), 4.29-4.13 (m, 1H), 2.78-2.63 (m, 5H), 2.55 (br dd, J=4.5, 17.0 Hz, 1H), 2.26-2.12 (m, 1H), 1.85 (br dd, J=4.6, 8.0 Hz, 2H), 1.45 (d, J=1.4 Hz, 9H)

### Procedure for preparation of ethyl 5-[tert-butoxycarbonyl(methyl)amino]-4,5,6,7-tetrahydrobenzothiophene-2-carboxylate (3)

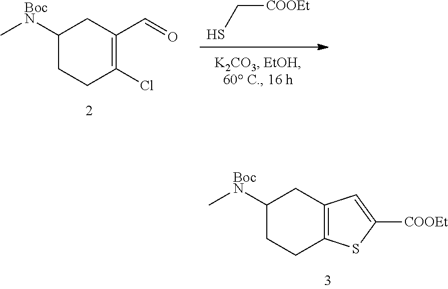 [SMILES: CN(C(=O)OC(C)(C)C)C1CCC(Cl)=C(C=O)C1 ; CCOC(=O)CS ; CCOC(=O)c1cc2c(s1)CCC(N(C)C(=O)OC(C)(C)C)C2]

To a solution of tert-butyl N-(4-chloro-3-formyl-cyclohex-3-en-1-yl)-N-methyl-carbamate (2) (25 g, 91.3 mmol, 1 eq) and K₂CO₃ (18.9 g, 136 mmol, 1.5 eq) in EtOH (200 mL) was added ethyl 2-sulfanylacetate (12.28 g, 102.19 mmol, 11.20 mL, 1.12 eq). The mixture was stirred at 60° C. for 16 hr. The reaction mixture was then filtered and the filtrate was concentrated. The residue was purified by flash silica gel chromatography (ISCO; 80 g SepaFlash Silica Flash Column, eluent 0~20% Ethyl acetate/Petroleum ether gradient @40 mL/min). Compound ethyl 5-[tert-butoxycarbonyl(methyl)amino]-4,5,6,7-tetrahydrobenzothiophene-2-carboxylate (3) (25 g, 73.65 mmol, 80.65% yield) was obtained as a yellow oil. LCMS: m/z [M+H-tBu]⁺=284.0. ¹H NMR (400 MHz, CHLOROFORM-d) δ 7.44 (s, 1H), 4.50-4.25 (m, 3H), 2.99-2.90 (m, 2H), 2.81 (s, 3H), 2.76-2.62 (m, 2H), 2.01-1.89 (m, 2H), 1.48 (s, 9H), 1.36 (t, J=7.1 Hz, 3H).

### Procedure for preparation of 5-[tert-butoxycarbonyl(methyl)amino]-4,5,6,7-tetrahydrobenzothiophene-2-carboxylic acid (4)

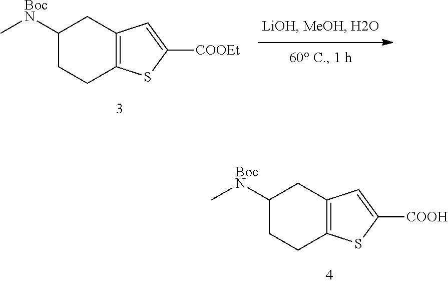 [SMILES: CCOC(=O)c1cc2c(s1)CCC(N(C)C(=O)OC(C)(C)C)C2 ; "LiOH, MeOH, H2O" ; CN(C(=O)OC(C)(C)C)C1CCc2sc(C(=O)O)cc2C1]

To a solution of ethyl 5-[tert-butoxycarbonyl(methyl)amino]-4,5,6,7-tetrahydrobenzothiophene-2-carboxylate (3) (25 g, 73.65 mmol, 1 eq) in MeOH (150 mL) and H₂O (150 mL) was added LiOH·H₂O (12.36 g, 294.60 mmol, 4 eq). The mixture was stirred at 60° C. for 1 hr. The reaction mixture was concentrated under vacuum to remove MeOH. The aqueous phase pH was adjusted to ~7 with HCl (1 mol/L in water), then adjusted pH to 3-4 with aq. sat. citric acid and filtered. The filter cake was washed with water (50 mL) and dried under vacuum. The crude product 5-[tert-butoxycarbonyl(methyl)amino]-4,5,6,7-tetrahydrobenzothiophene-2-carboxylic acid (4) (20 g, 64.23 mmol, 87.21% yield) was isolated as a white solid and was used into the next step without further purification. LCMS: m/z [M+H-tBu]⁺=256.0. ¹H NMR (400 MHz, CHLOROFORM-d) δ 7.54-7.46 (m, 1H), 4.57-4.14 (m, 1H), 3.06-2.90 (m, 2H), 2.87-2.62 (m, 5H), 2.06-1.86 (m, 2H), 1.49 (s, 9H).

### Procedure for preparation of tert-butyl N-methyl-N-(4,5,6,7-tetrahydrobenzothiophen-5-yl)carbamate (5)

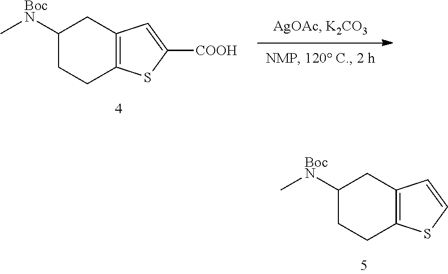

To a solution of 5-[tert-butoxycarbonyl(methyl)amino]-4,5,6,7-tetrahydrobenzothiophene-2-carboxylic acid (4) (10 g, 32.11 mmol, 1 eq) in NMP (100 mL) was added AgOAc (533 mg, 3.21 mmol, 0.1 eq) and K₂CO₃ (13.31 g, 96.3 mmol, 3 eq). The mixture was stirred at 120° C. for 2 hr. The reaction mixture was diluted with H₂O 200 mL and extracted with EtOAc (100 mL*3). The combined organic layers were washed with aq. sat.NaCl (100 mL*3), dried over anhydrous Na₂SO₄, filtered and concentrated under reduced pressure to give a residue. The residue was purified by flash silica gel chromatography (ISCO®; 40 g SepaFlash® Silica Flash Column, eluent 0~15% Ethyl acetate/Petroleum ether gradient @40 mL/min). Compound tert-butyl N-methyl-N-(4,5,6,7-tetrahydrobenzothiophen-5-yl)carbamate (5) (7.3 g, 27.30 mmol, 85.02% yield) was obtained as a white solid. ¹H NMR (400 MHz, CHLOROFORM-d) δ 7.09 (br d, J=5.0 Hz, 1H), 6.74 (d, J=5.0 Hz, 1H), 4.56-4.19 (m, 1H), 3.00-2.88 (m, 2H), 2.85-2.64 (m, 5H), 2.01-1.88 (m, 2H), 1.48 (s, 9H)

### Procedure for preparation of N-methyl-4,5,6,7-tetrahydrobenzothiophen-5-amine hydrochloride enantiomer 1 (11)

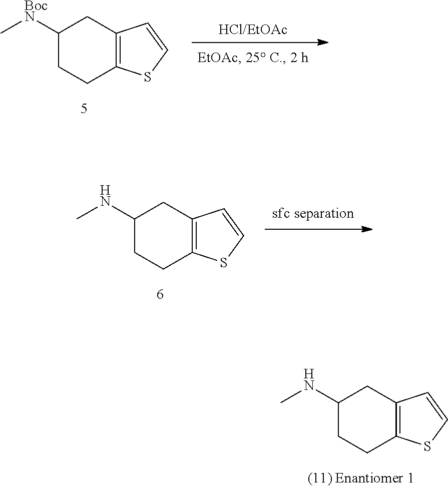 [SMILES: CN(C(=O)OC(C)(C)C)C1CCc2sccc2C1 ; "HCl/EtOAc" ; CNC1CCc2sccc2C1 ; "sfc separation" ; CNC1CCc2sccc2C1]

To a solution of tert-butyl N-methyl-N-(4,5,6,7-tetrahydrobenzothiophen-5-yl)carbamate (5) (2.5 g, 9.35 mmol, 1 eq) in EtOAc (5 mL) was added HCl/EtOAc (4 M, 5 mL, 2.14 eq). The mixture was stirred at 25° C. for 2 hr. The mixture was concentrated in vacuo. The reaction mixture was diluted with H₂O (20 mL), then the mixture was adjusted pH to 8~9 by aq. sat. NaHCO₃. and extracted with DCM:MeOH=10:1 (10 mL*5). The combined organic layers were washed with aq. sat. NaCl (20 mL), dried over anhydrous Na₂SO₄, filtered and concentrated under reduced pressure to give racemic N-methyl-4,5,6,7-tetrahydrobenzothiophen-5-amine (6) as a free base This material was separated by SFC (column: Phenomenex-Cellulose-2 (250 mm*50 mm, 10 μm); mobile phase: [CO₂-MeOH (0.1% NH₃H₂O)]; B %:25%, isocratic elution mode). The fractions contained desired enantiomer (peak 1 in SFC) were concentrated. The residue was dissolved with EtOAc (5 ml), followed addition of 4N HCl/EtOAc (5 ml). That resulted in the formation of a solid suspension. The mixture was filtered and the cake was washed with EtOAc (10 mL) to afford N-methyl-4,5,6,7-tetrahydrobenzothiophen-5-amine hydrochloride enantiomer 1 (11) (669.24 mg, 3.28 mmol, 35.08% yield) as a white solid. LCMS: m/z [M+H]⁺=168.0. ¹H NMR (400 MHz, DMSO-d6) δ 7.32 (d, J=5.1 Hz, 1H), 6.83 (d, J=5.1 Hz, 1H), 3.43-3.36 (m, 1H), 3.09 (dd, J=5.3, 15.6 Hz, 1H), 2.96-2.87 (m, 1H), 2.83-2.65 (m, 2H), 2.60 (s, 3H), 2.31-2.23 (m, 1H), 1.86 (tdd, J=5.7, 11.5, 17.7 Hz, 1H)

Alternative work-up to obtain racemic N-methyl-4,5,6,7-tetrahydrobenzothiophen-5-amine (6) as free base: Racemic N-methyl-4,5,6,7-tetrahydrobenzothiophen-5-amine hydrochloride (1.2 g) obtained from the HCl boc deprotection step was added in MeOH (10 mL) to form a suspension. Then 3 g of anion exchange resin (AMBERLITE(R)IRA-400 (CL, CAS: 122560-63-8) was added under stirring. The solution became clear immediately. The pH was about 8~9. Then the mixture was filtered and the filtrate was concentrated to give racemic N-methyl-4,5,6,7-tetrahydrobenzothiophen-5-amine (6) as a light yellow oil. (1.0 g).

### Synthetic Scheme for the preparation of N-methyl-4, 5, 6, 7-tetrahydro-2-benzothiophen-5-amine hydrochloride enantiomer 1 (13) and enantiomer 2 (14)

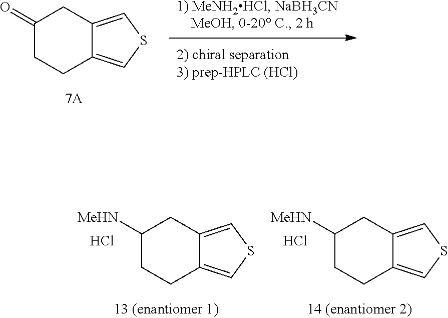

### Procedure for the preparation of N-methyl-4, 5, 6, 7-tetrahydro-2-benzothiophen-5-amine hydrochloride enantiomer 1 (13) and enantiomer 2 (14)

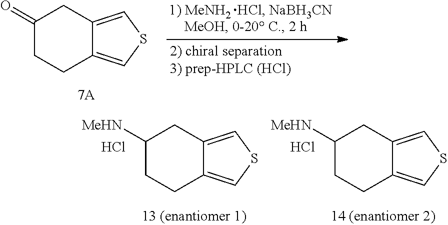 [SMILES: O=C1CCc2cscc2C1 ; Cl ; CNC1CCc2cscc2C1 ; Cl ; CNC1CCc2cscc2C1]

To a solution of 6, 7-dihydro-4H-2-benzothiophen-5-one (7A) (1.72 g, 11.3 mmol, 1 eq) and methylamine hydrochloride (3.81 g, 56.5 mmol, 5 eq) in MeOH (20 mL) was added NaBH₃CN (1.42 g, 22.6 mmol, 2 eq) at 0° C. The mixture was stirred at 20° C. for 2 hr. The reaction mixture was quenched by pouring into aq. sat. NaHCO₃ (20 mL), followed by extracting with EtOAc (10 mL*3). The combined organic layers were dried over Na₂SO₄, filtered, and concentrated in vacuo. The residue was purified by flash silica gel chromatography (ISCO®; 20 g SepaFlash® Silica Flash Column, eluent 0~90% Ethyl acetate/Petroleum ether) to give a racemate, which was separated by prep-Chiral-SFC (column: Phenomenex-Cellulose-2 (250 mm*30 mm, 10 μm); mobile phase A: supercritical CO₂; mobile phase B: 0.1% NH₃H₂O in EtOH; Gradient: isocratic 45% B; 3.6 min). The two enantiomers were further purified by prep-HPLC (HCl) to afford N-methyl-4,5,6,7-tetrahydro-2-benzothiophen-5-amine hydrochloride enantiomer 1 (13) (93.53 mg, 553.56 μmol, 46.13% yield) as a white solid and N-methyl-4, 5, 6, 7-tetrahydro-2-benzothiophen-5-amine hydrochloride enantiomer 2 (14) (67.26 mg, 398.08 μmol, 33.17% yield) as a white solid.

Enantiomer 1 (13): ¹H NMR (400 MHz, METHANOL-d₄) δ 7.19-7.09 (m, 1H), 7.05 (br s, 1H), 3.53-3.40 (m, 1H), 3.34-3.33 (m, 1H), 3.05-2.94 (m, 1H), 2.88-2.71 (m, 5H), 2.34-2.21 (m, 1H), 1.95-1.79 (m, 1H). LCMS: m/z [M+H]⁺=168.0.

Enantiomer 2 (14): ¹H NMR (400 MHz, METHANOL-d₄) δ 7.16-7.09 (m, 1H), 7.05 (br s, 1H), 3.53-3.39 (m, 1H), 3.37-3.32 (m, 1H), 3.05-2.94 (m, 1H), 2.86-2.71 (m, 5H), 2.32-2.21 (m, 1H), 1.93-1.78 (m, 1H). LCMS: m/z [M+H]⁺=168.0.

### Synthetic scheme for the synthesis of N,N-dimethyl-4,5,6,7-tetrahydrobenzothiophen-6-amine hydrochloride enantiomer 1 (15) and enantiomer 2 (16)

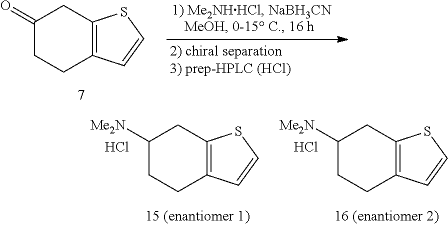 [SMILES: O=C1CCc2ccsc2C1 ; Cl ; CN(C)C1CCc2ccsc2C1 ; Cl ; CN(C)C1CCc2ccsc2C1]

### Procedure for the preparation of N,N-dimethyl-4,5,6,7-tetrahydrobenzothiophen-6-amine hydrochloride enantiomer 1 (15) and enantiomer 2 (16)

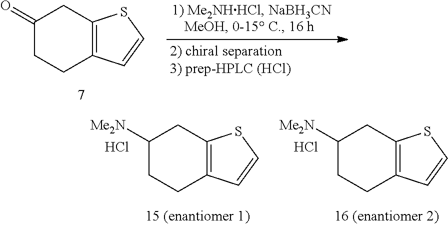 [SMILES: O=C1CCc2ccsc2C1 ; Cl ; CN(C)C1CCc2ccsc2C1 ; Cl ; CN(C)C1CCc2ccsc2C1]

To a mixture of 5,7-dihydro-4H-benzothiophen-6-one (7) (1.0 g, 6.57 mmol, 1 eq) in MeOH (20 mL) was added dimethylamine hydrochloride (2.14 g, 26.3 mmol, 4 eq). The resulting mixture was stirred at 15° C. for 1 hr before being treated with NaBH₃CN (826 mg, 13.1 mmol, 2 eq) at 0° C., and the stirring was continued at 15° C. for 16 hr. The reaction mixture was poured into sat. aq. NaHCO₃ solution (30 mL), followed by extracting with ethyl acetate (30 mL*3). The combined organic layers were dried over Na₂SO₄, filtered, and concentrated under reduced pressure. The crude product was purified by reversed-phase column chromatography (0.1% HCl condition) to give the racemate, which was separated by prep-Chiral-SFC (Column: Phenomenex-Cellulose-2 (250 mm*30 mm, 10 μm); Mobile phase A: supercritical CO₂; Mobile phase B: 0.1% NH₃H₂O in MeOH; Gradient: isocratic 35% B; 4.4 min). The two enantiomers were further purified by prep-HPLC (water(HCl)-ACN) to afford N,N-dimethyl-4,5,6,7-tetrahydrobenzothiophen-6-amine hydrochloride (enantiomer 1: 15) (113.63 mg, 521.82 μmol, 7.94% yield) as an off-white solid and N,N-dimethyl-4,5,6,7-tetrahydrobenzothiophen-6-amine hydrochloride (enantiomer 2: 16) (116.87 mg, 536.70 μmol, 8.17% yield) as an off-white solid.

Enantiomer 1 (15): ¹H NMR (400 MHz, METHANOL-d₄) δ 7.22 (d, J=5.1 Hz, 1H), 6.79 (d, J=5.3 Hz, 1H), 3.80-3.65 (m, 1H), 3.30-3.24 (m, 1H), 3.12-3.03 (m, 1H), 2.96 (s, 6H), 2.92-2.90 (m, 1H), 2.83-2.72 (m, 1H), 2.39-2.30 (m, 1H), 2.03-1.92 (m, 1H). LCMS: m/z [M+H]⁺=182.0.

Enantiomer 2 (16): ¹H NMR (400 MHz, METHANOL-d₄) δ 7.22 (d, J=5.1 Hz, 1H), 6.79 (d, J=5.1 Hz, 1H), 3.77-3.64 (m, 1H), 3.30-3.25 (m, 1H), 3.11-3.03 (m, 1H), 2.96 (s, 6H), 2.92-2.90 (m, 1H), 2.83-2.72 (m, 1H), 2.39-2.31 (m, 1H), 2.01-1.92 (m, 1H). LCMS: m/z [M+H]⁺=182.1.

### Synthetic scheme for the synthesis of 1-(4,5,6,7-tetrahydrobenzothiophen-6-yl)pyrrolidine hydrochloride enantiomer 1 (17) and enantiomer 2 (18)

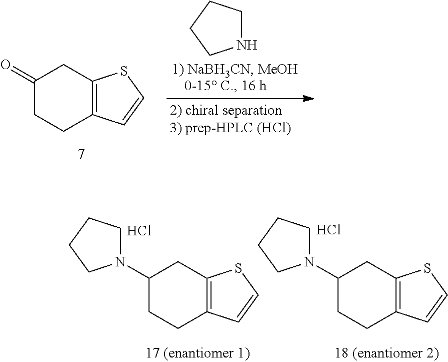

### Procedure for the preparation of 1-(4,5,6,7-tetrahydrobenzothiophen-6-yl)pyrrolidine hydrochloride enantiomer 1 (17) and enantiomer 2 (18)

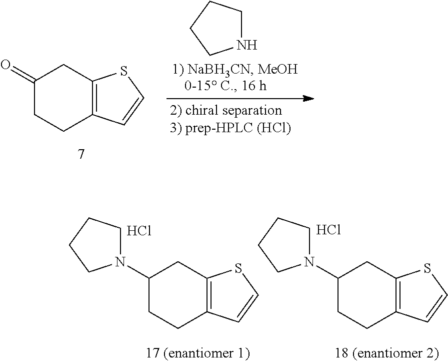

To a mixture of 5,7-dihydro-4H-benzothiophen-6-one (7) (670 mg, 4.40 mmol, 1 eq) in MeOH (20 mL) was added pyrrolidine (939 mg, 13.2 mmol, 1.10 mL, 3.00 eq), the mixture was stirred at 15° C. for 1 hr. Then NaBH₃CN (553 mg, 8.80 mmol, 2 eq) was added at 0° C., the resulting mixture was stirred at 15° C. for 16 hr. The reaction mixture was poured into sat.NH₄Cl (30 mL), and the resulting mixture was extracted with ethyl acetate (30 mL*4). The combined organic layers were washed with brine (20 mL*2), dried over Na₂SO₄, filtered, and concentrated under reduced pressure. The crude product was purified by reversed-phase column chromatography (0.1% HCl condition) to give a racemate, which was separated by prep-Chiral-SFC (Column: DAICEL CHIRALPAK AD (250 mm*30 mm, 10 μm); Mobile phase A: supercritical CO₂; Mobile phase B: 0.1% NH₃H₂O in IPA; Gradient: isocratic 30% B; 3 min). The two enantiomers were separately further purified by prep-HPLC (water(HCl)-ACN) to afford 1-(4,5,6,7-tetrahydrobenzothiophen-6-yl)pyrrolidine hydrochloride salt (enantiomer 1: 17) (134.64 mg, 552.27 μmol, 12.55% yield) as an off-white solid and 1-(4,5,6,7-tetrahydrobenzothiophen-6-yl)pyrrolidine hydrochloride salt (enantiomer 2: 18) (125.27 mg, 513.83 μmol, 11.67% yield) as an off-white solid.

Enantiomer 1 (17): ¹H NMR (400 MHz, METHANOL-d₄) δ 7.22 (d, J=5.1 Hz, 1H), 6.80 (d, J=5.1 Hz, 1H), 3.84-3.70 (m, 2H), 3.68-3.57 (m, 1H), 3.39 (dd, J=5.1, 15.7 Hz, 1H), 3.29-3.20 (m, 2H), 3.09-2.98 (m, 1H), 2.94-2.86 (m, 1H), 2.85-2.70 (m, 1H), 2.45-2.34 (m, 1H), 2.25-2.15 (m, 2H), 2.10-1.93 (m, 3H). LCMS: m/z [M+H]⁺=208.0.

Enantiomer 2 (18): ¹H NMR (400 MHz, METHANOL-d₄) δ 7.23 (d, J=5.0 Hz, 1H), 6.80 (d, J=5.1 Hz, 1H), 3.79-3.70 (m, 2H), 3.68-3.57 (m, 1H), 3.39 (dd, J=5.0, 15.8 Hz, 1H), 3.29-3.20 (m, 2H), 3.02 (dd, J=9.7, 15.7 Hz, 1H), 2.94-2.86 (m, 1H), 2.84-2.72 (m, 1H), 2.45-2.35 (m, 1H), 2.25-2.15 (m, 2H), 2.09-1.94 (m, 3H). LCMS: m/z [M+H]⁺=208.1.

### Synthetic Scheme for the synthesis of N-methyl-5,6-dihydro-4H-cyclopenta[b]thiophen-5-amine hydrochloride enantiomer 1 (19) and enantiomer 2 (20)

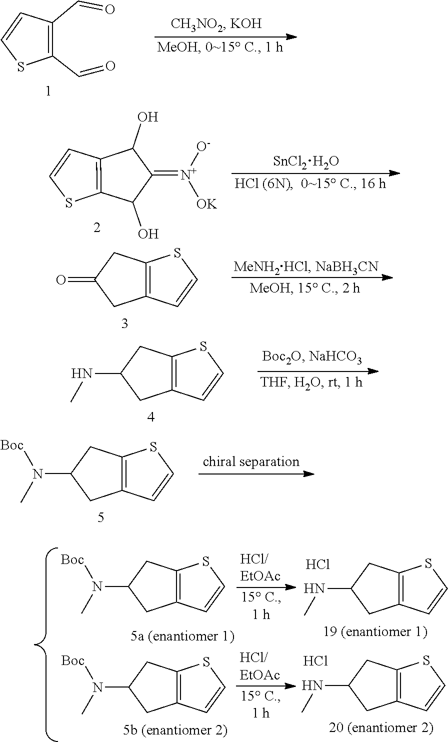

### Procedure for the preparation of [(4,6-dihydroxy-4,6-dihydrocyclopenta[b]thiophen-5-ylidene)-oxido-ammonio]oxypotassium (2)

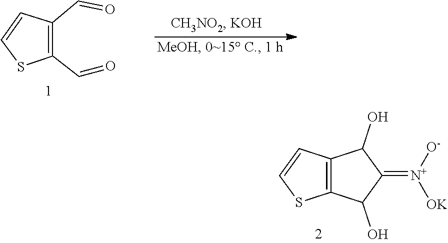

The solution of thiophene-2,3-dicarbaldehyde (1) (10.0 g, 71.3 mmol, 1 eq) in MeOH (40 mL) was added to a solution of KOH (10.0 g, 178 mmol, 2.5 eq) in MeOH (140 mL) at 0° C. Then a solution of nitromethane (6.10 g, 99.9 mmol, 5.40 mL, 1.4 eq) in MeOH (20 mL) was added dropwise over 0.2 h. The mixture was stirred at 15° C. for 0.5 h. The precipitate was collected by filtration, washed with cold MeOH, and dried to give [(4,6-dihydroxy-4,6-dihydro cyclopenta[b]thiophen-5-ylidene)-oxido-ammonio]oxypotassium (2) (17.0 g, 39.9%) as a gray solid.

### Procedure for the preparation of 4,6-dihydrocyclopenta[b]thiophen-5-one (3)

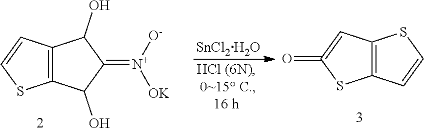

To a solution of SnCl₂·2H₂O (70.7 g, 313 mmol, 5 eq) in 6 N HCl (300 mL) was added [(4,6-dihydroxy-4,6-dihydrocyclopenta[b]thiophen-5-ylidene)-oxido-ammonio]oxypotassium (2) (15.0 g, 62.7 mmol, 1 eq) at 0° C. portion-wise, and the resulting mixture was stirred at 15° C. for 16 hr. The reaction mixture was diluted with H₂O (100 mL) and extracted with EtOAc (100 mL*2). The combined organic layers were washed with brine (100 mL), dried over anhydrous Na₂SO₄, filtered, and concentrated under reduced pressure. The residue was purified by flash silica gel chromatography (ISCO®; 80 g SepaFlash® Silica Flash Column, Eluting with 0~15% Ethyl acetate/Petroleum ether) to give 4,6-dihydrocyclopenta [b]thiophen-5-one (2.41 g, 17.44 mmol, 27.82% yield) as a yellow solid. ¹H NMR (400 MHz, CDCl₃) δ 7.27 (d, J=4.8 Hz, 1H), 6.98 (d, J=4.8 Hz, 1H), 3.56 (s, 2H), 3.43 (s, 2H).

### Procedure for the preparation of thiophen-5-one (3)N-methyl-5,6-dihydro-4H-cyclopenta[b]thiophen-5-amine (4)

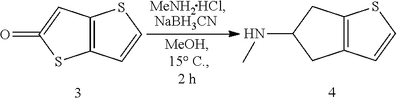

To a solution of 4,6-dihydrocyclopenta[b]thiophen-5-one (3) (1.0 g, 7.24 mmol, 1 eq) and methylamine hydrochloride (2.44 g, 36.2 mmol, 5 eq) in MeOH (10 mL) was added NaBH₃CN (2.27 g, 36.2 mmol, 5 eq). The reaction mixture was stirred at 15° C. for 2 hr before being quenched with H₂O (20 mL), followed by extracting with EtOAc (10 mL*2). The combined organic layers were washed with brine (10 mL), dried over anhydrous Na₂SO₄, filtered, and concentrated under reduced pressure. The residue was purified by flash silica gel chromatography to provide N-methyl-5,6-dihydro-4H-cyclopenta[b]thiophen-5-amine (4) (430 mg, 2.74 mmol, 37.92% yield) as a yellow oil. ¹H NMR (400 MHz, CDCl₃) δ 7.25 (d, J=4.8 Hz, 1H), 6.83 (d, J=4.8 Hz, 1H), 4.30-4.22 (m, 1H), 3.41 (dd, J=8.0, 16.4 Hz, 1H), 3.26 (dd, J=8.0, 15.6 Hz, 1H), 3.16 (dd, J=5.6, 16.0 Hz, 1H), 3.00 (dd, J=5.6, 15.6 Hz, 1H), 2.73 (s, 3H).

### Procedure for the preparation of N-methyl-5,6-dihydro-4H-cyclopenta[b]thiophen-5-amine (4) and tert-butyl N-(5,6-dihydro-4H-cyclopenta[b]thiophen-5-yl)-N-methyl-carbamate enantiomers 1 and 2 (5a, 5b)

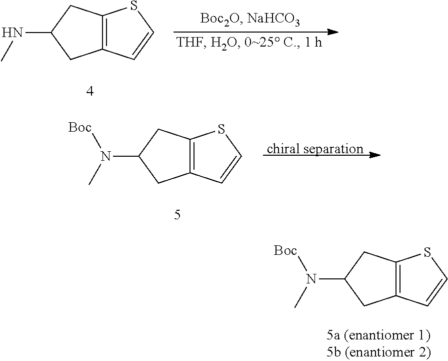

To a solution of N-methyl-5,6-dihydro-4H-cyclopenta[b]thiophen-5-amine (4) (430 mg, 2.81 mmol, 1 eq) in THF (2 mL) and H₂O (1 mL) was added NaHCO₃ (55 mg, 0.65 mmol, 2 eq) and Boc₂O (920 mg, 4.22 mmol, 1.5 eq). The reaction mixture was stirred at rt for 1 hr. The reaction mixture was poured into water (5 mL) and extracted with EtOAc (10 mL*3). The combined organic phase was washed with brine, dried over anhydrous sodium sulfate, filtered, and concentrated under vacuum. The crude product was subjected to prep-chiral-SFC (Column: DAICEL CHIRALPAK IF (250 mm*30 mm, 10 μm); Mobile phase A: supercritical CO₂; Mobile phase B: MeCN; Gradient: isocratic 15% B, 3.3 min) to afford the 1^{st} enantiomer (5a) (180 mg, 0.71 mmol, 25.3% yield) and the 2^{nd} enantiomer (5b) (780 mg, crude), both as white solids.

5a: ¹H NMR (400 MHz, CDCl₃) δ 7.18 (d, J=5.2 Hz, 1H), 6.81 (d, J=4.8 Hz, 1H), 5.58-5.27 (m, 1H), 3.17 (dd, J=8.8, 16.0 Hz, 1H), 3.02 (dd, J=8.8, 15.2 Hz, 1H), 2.89 (dd, J=5.6, 15.6 Hz, 1H), 2.77-2.69 (m, 4H), 1.48 (s, 9H).

5b: ¹H NMR (400 MHz, CDCl₃) δ 7.18 (d, J=4.8 Hz, 1H), 6.81 (d, J=5.2 Hz, 1H), 5.56-5.29 (m, 1H), 3.17 (dd, J=8.4, 16.0 Hz, 1H), 3.02 (dd, J=7.6, 16.0 Hz, 1H), 2.89 (dd, J=6.4, 16.4 Hz, 1H), 2.77-2.71 (m, 4H), 1.47 (s, 9H).

### Procedure for the preparation of N-methyl-5,6-dihydro-4H-cyclopenta[b]thiophen-5-amine hydrochloride enantiomer 1 (19)

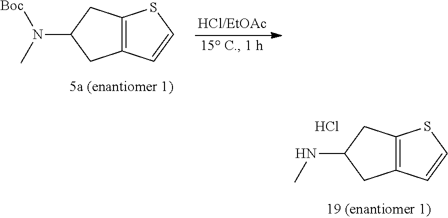

To a solution of tert-butyl N-(5,6-dihydro-4H-cyclopenta[b]thiophen-5-yl)-N-methyl-carbamate enantiomer 1 (5a) (180 mg, 710 μmol, 1 eq) in EtOAc (2 mL) was added HCl/EtOAc (4 M, 2 mL, 11.26 eq). The mixture was stirred at 15° C. for 1 hr. The precipitate was collected by filtration to afford N-methyl-5,6-dihydro-4H-cyclopenta[b]thiophen-5-amine hydrochloride enantiomer 1 (19) (104 mg, 548.22 μmol, 77.16% yield) as a yellow solid. ¹H NMR (400 MHz, Methanol-d4) δ 7.35 (d, J=5.6 Hz, 1H), 6.87 (d, J=5.2 Hz, 1H), 4.41-4.31 (m, 1H), 3.49-3.38 (m, 1H), 3.28-3.20 (m, 1H), 3.09-3.05 (m, 1H), 2.94-2.84 (m, 1H), 2.76 (s, 3H). LCMS: m/z [M+H]⁺=153.9.

### Procedure for preparation of the N-methyl-5,6-dihydro-4H-cyclopenta[b]thiophen-5-amine hydrochloride enantiomer 2 (20)

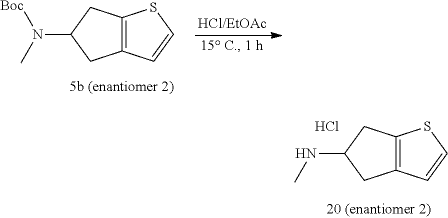

To a solution of tert-butyl N-(5,6-dihydro-4H-cyclopenta[b]thiophen-5-yl)-N-methyl-carbamate enantiomer 2 (5b) (780 mg, crude, 1 eq) in EtOAc (2 mL) was added HCl/EtOAc (4 M, 2 mL). The mixture was stirred at 15° C. for 1 hr. The precipitate was collected by filtration to afford N-methyl-5,6-dihydro-4H-cyclopenta[b]thiophen-5-amine hydrochloride enantiomer 2 (20) (125 mg, 658.92 μmol, 21.40% yield) a yellow solid. ¹H NMR (400 MHz, Methanol-d4) δ 7.35 (d, J=5.2 Hz, 1H), 6.87 (d, J=4.8 Hz, 1H), 4.39-4.31 (m, 1H), 3.42 (dd, J=8.4, 12.4 Hz, 1H), 3.29-3.24 (m, 1H), 3.05 (dd, J=5.2, 16.0 Hz, 1H), 2.89 (dd, J=4.8, 16.0 Hz, 1H), 2.76 (s, 3H). LCMS: m/z [M+H]⁺=153.9.

### Synthetic scheme for the preparation of N-methyl-5,6-dihydro-4H-cyclopenta[c]thiophen-5-amine hydrochloride (21)

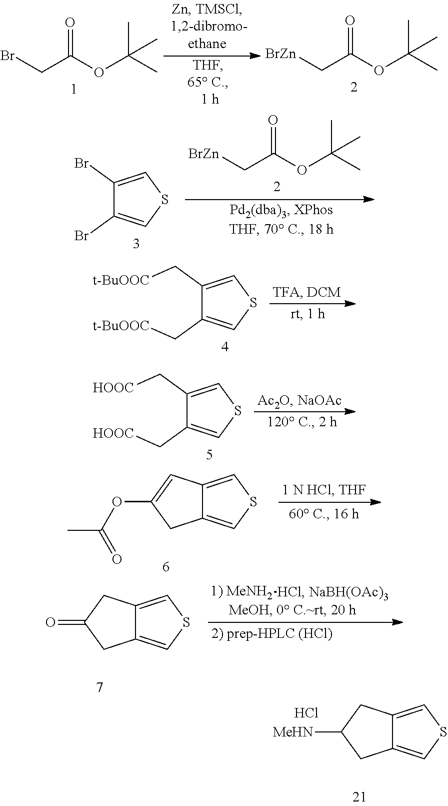

### Procedure for the preparation of bromo-(2-tert-butoxy-2-oxo-ethyl)zinc (2)

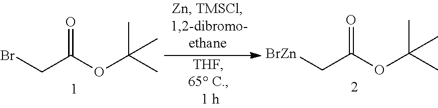

To a three-necked round-bottomed flask were added zinc (20.76 g, 317 mmol, 1.55 eq) and THF (600 mL). Then 1,2-dichloroethane (4.06 g, 41.0 mmol, 3.25 mL, 0.2 eq) and TMSCl (2.23 g, 20.5 mmol, 2.60 mL, 0.1 eq) were added. The resulting mixture was stirred at 15-20° C. for 0.5 hr under N₂ atmosphere before being heated to 65° C. tert-Butyl 2-bromoacetate (40.0 g, 205 mmol, 30.3 mL, 1 eq) was added drop-wise, and the stirring was continued at 65° C. for 1 hr. The mixture was cooled to ambient temperature to afford bromo-(2-tert-butoxy-2-oxo-ethyl) zinc in THF.

### Procedure for the preparation of tert-butyl 2-[4-(2-tert-butoxy-2-oxo-ethyl)-3-thienyl]acetate (4)

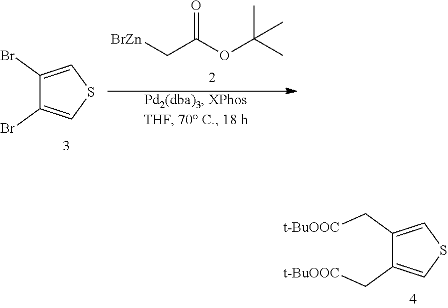

To the mixture of 3,4-dibromothiophene (3) (10.0 g, 41.33 mmol, 1 eq) and 400 mL supernatant of the above prepared bromo-(2-tert-butoxy-2-oxo-ethyl) zinc in THF was added Pd₂(dba)₃ (3.79 g, 4.13 mmol, 0.1 eq) and XPhos (3.94 g, 8.27 mmol, 0.2 eq). The reaction vessel was evacuated/backfilled with N₂ for three times. The resulting mixture was stirred at 70° C. for 18 hr. The reaction mixture was cooled to ambient temperature, quenched with sat. NH₄Cl (500 mL), and extracted with EtOAc (3*200 mL). The combined organic extracts were dried over Na₂SO₄, filtered, and concentrated under vacuum. The residue was purified by flash silica gel chromatography (eluting with 1:10 Ethyl acetate/Petroleum ether) to afford tert-butyl 2-[4-(2-tert-butoxy-2-oxo-ethyl)-3-thienyl]acetate (4) (9.7 g, 31.05 mmol, 75.11% yield) as a yellow oil. ¹H NMR (400 MHz, CHLOROFORM-d) δ 7.15 (s, 2H), 3.53 (s, 4H), 1.46 (s, 18H).

### Procedure for the preparation of 2-[4-(carboxymethyl)-3-thienyl]acetic acid (5)

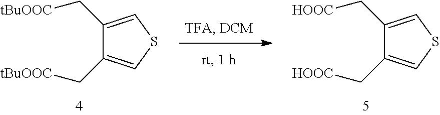

To the mixture of tert-butyl 2-[4-(2-tert-butoxy-2-oxo-ethyl)-3-thienyl]acetate (4) (9.20 g, 29.45 mmol, 1 eq) in DCM (20 mL) was added TFA (20 mL). The resulting mixture was stirred at rt for 1 hr. The reaction mixture was concentrated under vacuum to give 2-[4-(carboxymethyl)-3-thienyl]acetic acid (5) (6.5 g, crude) as a yellow solid.

### Procedure for the preparation of 6H-cyclopenta[c]thiophen-5-yl acetate (6)

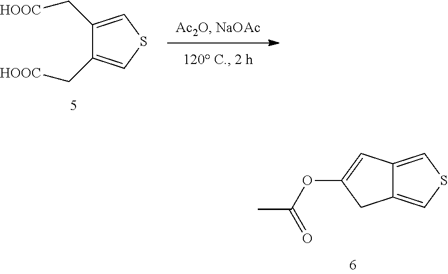

To a solution of 2-[4-(carboxymethyl)-3-thienyl]acetic acid (5) (1.00 g, 4.99 mmol, 1 eq) in Ac₂O (20 mL) was added NaOAc (1.23 g, 14.98 mmol, 3 eq), and the resulting mixture was stirred at 120° C. for 2 hr. The reaction mixture was cooled to ambient temperature, quenched by pouring into NaHCO₃ saturated solution (100 mL), and extracted with EtOAc (20 mL*3). The combined organic phase was washed with brine (10 mL), dried over anhydrous sodium sulfate, filtered and concentrated under vacuum. The residue was purified by flash silica gel chromatography (eluting with 1:10 Ethyl acetate/Petroleum ether) to afford 6H-cyclopenta[c]thiophen-5-yl acetate (6) (320 mg, 1.78 mmol, 35.55% yield) as a yellow solid. ¹H NMR (400 MHz, CHLOROFORM-d) δ 6.97 (d, J=0.8 Hz, 1H), 6.83 (d, J=2.1 Hz, 1H), 6.50 (s, 1H), 3.40 (s, 2H), 2.24 (s, 3H). LCMS: m/z [M+H]⁺=181.5.

### Procedure for the preparation of 4,6-dihydrocyclopenta[c]thiophen-5-one (7)

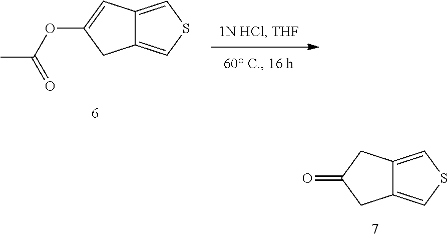

To a solution of 6H-cyclopenta[c]thiophen-5-yl acetate (6) (350 mg, 1.94 mmol, 1 eq) in THF (2 mL) was added 1 M HCl (2 mL), and the resulting mixture was stirred at 60° C. for 16 hr. The reaction mixture was diluted with H₂O (10 mL) and extracted with EtOAc (10 mL*3). The combined organic layer was washed with sat.NaHCO₃ (10 mL), dried over Na₂SO₄, filtered and concentrated in vacuo. The residue was purified by flash silica gel chromatography (eluting with 1:5 Ethyl acetate/Petroleum ether) to afford 4,6-dihydrocyclopenta[c]thiophen-5-one (7) (220 mg, 1.59 mmol, 81.98% yield) as a yellow solid. ¹H NMR (400 MHz, CHLOROFORM-d) δ 7.09 (s, 2H), 3.44 (s, 4H)

### Procedure for the preparation of N-methyl-5,6-dihydro-4H-cyclopenta[c]thiophen-5-amine hydrochloride (21)

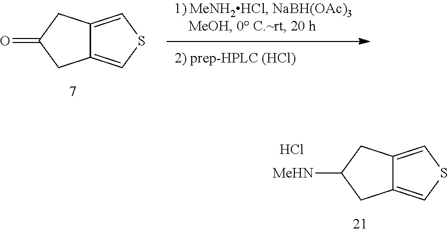 [SMILES: O=C1Cc2cscc2C1 ; Cl ; CNC1Cc2cscc2C1]

To a solution of 4,6-dihydrocyclopenta[c]thiophen-5-one (7) (200 mg, 1.45 mmol, 1 eq) in MeOH (6 mL) was added methylamine hydrochloride (293 mg, 4.34 mmol, 3 eq). The resulting mixture was stirred at 25° C. for 18 hr before being treated with NaBH(OAc)₃ (1.84 g, 8.68 mmol, 6 eq) at 0° C., and the stirring was continued at 25° C. for 2 hr. The reaction mixture was quenched with H₂O (20 mL) and extracted with EtOAc (3*10 mL). The combined organic extracts were dried over Na₂SO₄, filtered, and concentrated under vacuum. The residue was purified by prep-HPLC (column: 3_Phenomenex Luna C18 75*30 mm*3 um; mobile phase: [water(HCl)-ACN]; B %: 0%-8%, 8 min) to afford N-methyl-5,6-dihydro-4H-cyclopenta[c]thiophen-5-amine hydrochloride (21) (55.13 mg, 359.75 μmol, 24.86% yield) as a white solid. ¹H NMR (400 MHz, METHANOL-d₄) δ 7.05 (s, 2H), 4.27-4.16 (m, 1H), 3.25 (dd, J=7.5, 16.3 Hz, 2H), 2.85 (dd, J=6.1, 16.1 Hz, 2H), 2.75 (s, 3H). LCMS: m/z [M+H]⁺=154.0.

### Synthetic scheme for the preparation of 4-(4,5,6,7-tetrahydrobenzo[b]thiophen-6-yl)morpholine hydrochloride, enantiomer 1 (22) and enantiomer 2 (23)

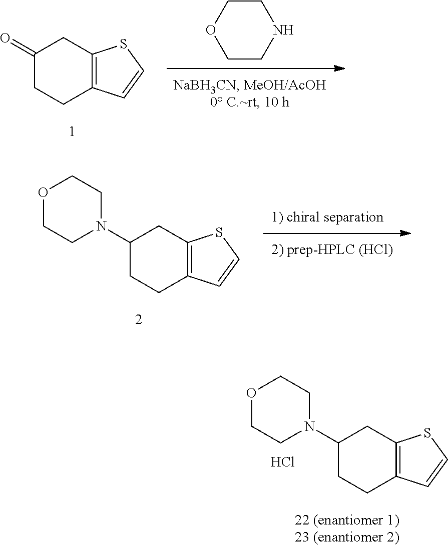 [SMILES: O=C1CCc2ccsc2C1 ; C1COCCN1 ; c1cc2c(s1)CC(N1CCOCC1)CC2 ; c1cc2c(s1)CC(N1CCOCC1)CC2 ; Cl]

### Procedure for the preparation of 4-(4,5,6,7-tetrahydrobenzo[b]thiophen-6-yl)morpholine enantiomer 1 (22) and enantiomer 2 (23)

To a solution of 5,7-dihydro-4H-benzothiophen-6-one (1) (2.00 g, 13.14 mmol, 1 eq) and morpholine (1.49 g, 17.08 mmol, 1.50 mL, 1.3 eq) in MeOH (30 mL) was added AcOH (1.18 g, 19.71 mmol, 1.13 mL, 1.5 eq). NaBH₃CN (2.06 g, 32.85 mmol, 2.5 eq) was then added at 0° C. The resulting mixture was stirred at 20° C. for 10 hr. The reaction mixture was diluted with H₂O (50 mL) and extracted with EtOAc (30 mL*2). The combined organic layers were washed with brine (20 mL), dried over anhydrous Na₂SO₄, filtered, and concentrated under reduced pressure. The residue was purified by flash silica gel chromatography to give the racemic 4-(4,5,6,7-tetrahydrobenzo[b]thiophen-6-yl)morpholine (2) (340 mg, 1.40 mmol, 10.66% yield) as a yellow oil, which was subjected to prep-chiral-SFC (column: DAICEL CHIRALCEL OD-H (250 mm*30 mm, 5 μm); mobile phase A: supercritical CO₂; mobile phase B: nHeptane-IPA (0.1% NH₃—H₂O)]; B %: isocratic 15% B for 3.4 min) to afford two enantiomers. Both enantiomers were further purified by prep-HPLC (HCl condition).

22 (enantiomer 1): 94.38 mg, 352.39 μmol, HCl salt, white solid. ¹H NMR (400 MHz, METHANOL-d₄) δ 7.23 (d, J=5.0 Hz, 1H), 6.79 (d, J=5.1 Hz, 1H), 4.12 (dd, J=2.6, 13.1 Hz, 2H), 3.93-3.80 (m, 2H), 3.77-3.66 (m, 1H), 3.65-3.52 (m, 2H), 3.42-3.34 (m, 2H), 3.31-3.26 (m, 1H), 3.13-3.04 (m, 1H), 3.01-2.91 (m, 1H), 2.84-2.71 (m, 1H), 2.49-2.40 (m, 1H), 2.00-1.90 (m, 1H). LCMS: m/z [M+H]⁺=224.0.

23 (enantiomer 2): 87.81 mg, 334.62 μmol, HCl salt, white solid. ¹H NMR (400 MHz, METHANOL-d₄) δ 7.23 (d, J=5.1 Hz, 1H), 6.79 (d, J=5.1 Hz, 1H), 4.12 (dd, J=2.8, 13.0 Hz, 2H), 3.92-3.81 (m, 2H), 3.77-3.66 (m, 1H), 3.66-3.52 (m, 2H), 3.42-3.34 (m, 2H), 3.31-3.27 (m, 1H), 3.14-3.04 (m, 1H), 3.00-2.90 (m, 1H), 2.83-2.71 (m, 1H), 2.48-2.39 (m, 1H), 2.00-1.90 (m, 1H). LCMS: m/z [M+H]⁺=224.1.

### Synthetic scheme for the preparation of N-methyl-7,8-dihydro-6H-cyclohepta[b]thiophen-7-amine hydrochloride (24), and N-methyl-5,6,7,8-tetrahydro-4H-cyclohepta[b]thiophen-7-amine hydrochloride, enantiomer 1 (25) and enantiomer 2 (26)

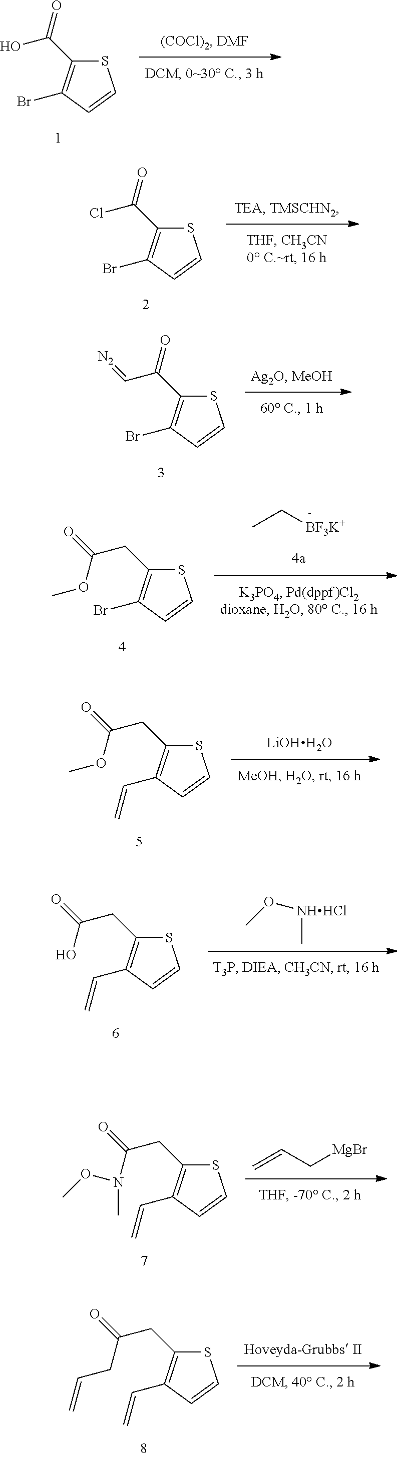

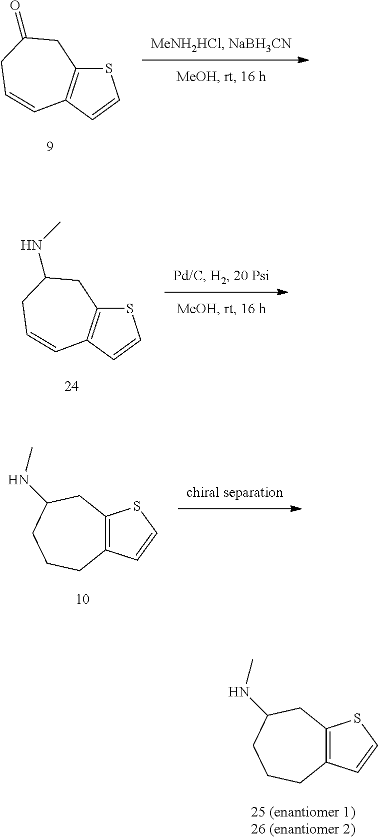

### Procedure for the preparation of 3-bromothiophene-2-carbonyl chloride (2)

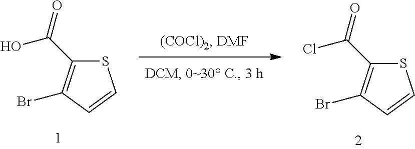 [SMILES: O=C(O)c1sccc1Br ; O=C(Cl)c1sccc1Br]

To the mixture of 3-bromothiophene-2-carboxylic acid (1) (20.0 g, 96.6 mmol, 1.00 eq) in DCM (50 mL) and DMF (141 mg, 1.93 mmol, 0.02 eq) was added (COCl)₂ (16.5 g, 130 mmol, 11.4 mL, 1.35 eq) dropwise at 0-5° C., and the resulting mixture was stirred at 30° C. for 3 hr. The reaction mixture was concentrated under vacuum to give 3-bromothiophene-2-carbonyl chloride (2) (22.4 g, crude) as a light-yellow solid.

### Procedure for the preparation of 1-(3-bromo-2-thienyl)-2-diazo-ethanone (3)

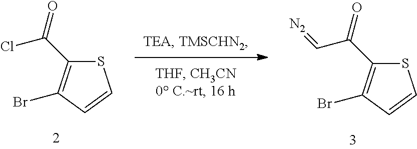

To the mixture of 3-bromothiophene-2-carbonyl chloride (2) (22.4 g crude) in CH₃CN (200 mL) and THF (200 mL) was added drop-wise TEA (9.77 g, 96.6 mmol, 13.4 mL, 1 eq) and diazomethyl(trimethyl)silane (2 M in hexane, 48.3 mL, 966 mmol, 1 eq) at 0° C. under N₂ atmosphere, and the resulting mixture was stirred at ambient temperature for 16 h. The reaction mixture was concentrated under vacuum. The residue was purified by flash silica gel chromatography (ISCO®; 80 g SepaFlash® Silica Flash Column, eluent 0~15% Ethyl acetate/Petroleum ether) to provide 1-(3-bromo-2-thienyl)-2-diazo-ethanone (3) (14 g, 60.6 mmol, 62.7% yield) as a yellow solid. ¹H NMR (400 MHz, CDCl₃) δ 7.54 (d, J=5.2 Hz, 1H), 7.10 (d, J=5.2 Hz, 1H), 6.55 (s, 1H).

### Procedure for the preparation of methyl 2-(3-bromo-2-thienyl)acetate (4)

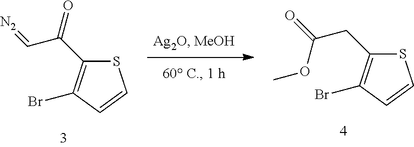

To the solution of 1-(3-bromo-2-thienyl)-2-diazo-ethanone (3) (14 g, 60.6 mmol, 1 eq) in MeOH (200 mL) was added Ag₂O (14.04 g, 60.59 mmol, 1 eq). The resulting mixture was stirred at 60° C. for 1 h. The reaction mixture was filtered, and the filtrate was concentrated under vacuum to give a residue which was purified by column chromatography (SiO₂, Petroleum ether/Ethyl acetate=100/0 to 15/1) to give methyl 2-(3-bromo-2-thienyl)acetate (4) (12.1 g, 51.5 mmol, 84.9% yield) as a yellow oil. ¹H NMR (400 MHz, CDCl₃) δ 7.25 (d, J=5.6 Hz, 1H), 6.97 (d, J=5.6 Hz, 1H), 3.85 (s, 2H), 3.76 (s, 3H).

### Procedure for the preparation of methyl 2-(3-vinyl-2-thienyl)acetate (5)

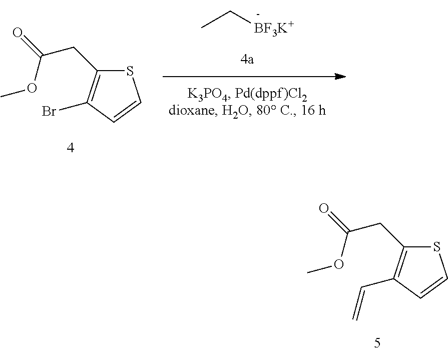

To the mixture of methyl 2-(3-bromo-2-thienyl)acetate (4) (12.1 g, 51.5 mmol, 1 eq), potassium vinyltrifluoroborate (15.2 g, 113 mmol, 2.2 eq) and K₃PO₄ (32.8 g, 154 mmol, 3 eq) in dioxane (120 mL) and H₂O (6 mL) was added Pd(dppf)Cl₂ (1.88 g, 2.57 mmol, 0.05 eq). The resulting mixture was stirred at 80° C. for 16 h under N₂ atmosphere. The reaction mixture was cooled to rt, filtered, and the filtrate was concentrated under vacuum to give a residue which was purified by column chromatography (silica gel, Petroleum ether/Ethyl acetate=100/0 to 20/1) to give methyl 2-(3-vinyl-2-thienyl)acetate (5) (6 g, 32.9 mmol, 63.9% yield). ¹H NMR (400 MHz, CDCl₃) δ 7.22-7.14 (m, 2H), 6.71 (dd, J=11.2, 17.2 Hz, 1H), 5.58 (dd, J=1.2, 17.2 Hz, 1H), 5.28 (dd, J=1.2, 11.2 Hz, 1H), 3.84 (s, 2H), 3.73 (s, 3H).

### Procedure for the preparation of 2-(3-vinyl-2-thienyl)acetic acid (6)

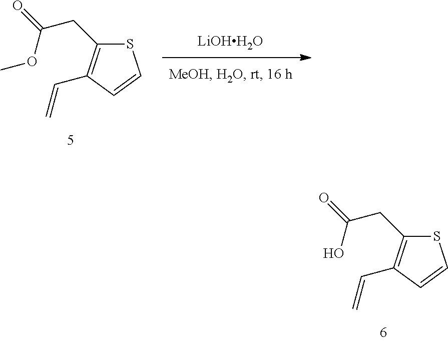

To the mixture of methyl 2-(3-vinyl-2-thienyl)acetate (5) (6 g, 32.9 mmol, 1 eq) in MeOH (15 mL) and H₂O (15 mL) was added LiOH·H₂O (2.76 g, 65.8 mmol, 2 eq). The resulting mixture was stirred at room temperature for 16 hr. The reaction mixture was concentrated under vacuum to remove MeOH, treated with 6 N HCl until pH 2~3, and extracted with EtOAc (15 mL*3). The combined organic phase was washed with brine (20 mL), dried over anhydrous sodium sulfate, filtered and concentrated under vacuum to give 2-(3-vinyl-2-thienyl)acetic acid (6) (5.5 g, 32.70 mmol, 99.31% yield) as a yellow solid. ¹H NMR (400 MHz, DMSO-d₆) δ 12.44 (br s, 1H), 7.37 (d, J=5.6 Hz, 1H), 7.29 (d, J=5.6 Hz, 1H), 6.73 (dd, J=11.2, 17.6 Hz, 1H), 5.62 (dd, J=1.2, 17.6 Hz, 1H), 5.22 (dd, J=1.2, 10.8 Hz, 1H), 3.82 (s, 2H).

### Procedure for the preparation of N-methoxy-N-methyl-2-(3-vinyl-2-thienyl)acetamide (7)

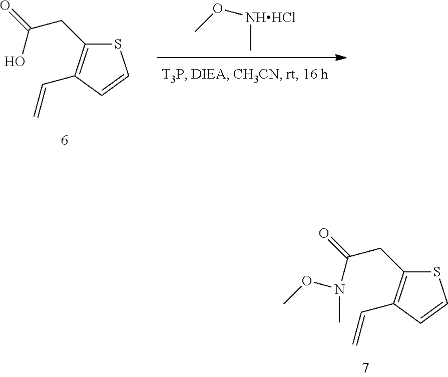

To the mixture of 2-(3-vinyl-2-thienyl)acetic acid (6) (5.5 g, 32.7 mmol, 1 eq), N-methoxymethanamine hydrochloride (4.78 g, 49.1 mmol, 1.5 eq) and DIEA (12.7 g, 98.1 mmol, 3 eq) in CH₃CN (50 mL) was added T₃P (33.3 g, 52.3 mmol, 50% in EtOAc, 1.6 eq), and the resulting mixture was stirred at 15° C. for 16 h. The reaction mixture was diluted with water (200 mL) and extracted with EtOAc (50 mL*3). The combined organic phase was washed with brine (100 mL), dried over anhydrous sodium sulfate, filtered and concentrated under vacuum. The residue was purified by column chromatography (SiO₂, Petroleum ether/Ethyl acetate=1/0 to 10/1) to give N-methoxy-N-methyl-2-(3-vinyl-2-thienyl)acetamide (7) (5 g, 23.7 mmol, 72.4% yield) as a yellow oil. ¹H NMR (400 MHz, CDCl₃) δ 7.21 (d, J=5.2 Hz, 1H), 7.17 (d, J=5.2 Hz, 1H), 6.79 (dd, J=10.8, 17.2 Hz, 1H), 5.59 (dd, J=1.2, 17.6 Hz, 1H), 5.27 (dd, J=1.2, 10.8 Hz, 1H), 4.00 (s, 2H), 3.69 (s, 3H), 3.23 (s, 3H).

### Procedure for the preparation of 1-(3-vinyl-2-thienyl)pent-4-en-2-one (8)

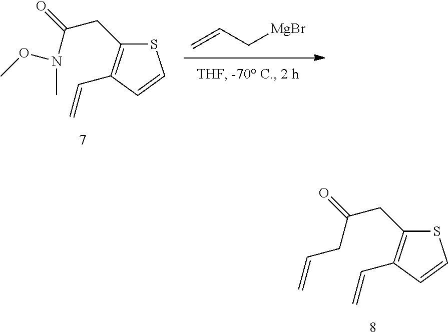

To the mixture of N-methoxy-N-methyl-2-(3-vinyl-2-thienyl)acetamide (7) (5.0 g, 23.7 mmol, 1 eq) in THF (150 mL) was added allylmagnesium bromide (1 M in THF, 24.9 mL, 24.9 mmol, 1.05 eq) at −70° C. under N₂ atmosphere, and the resulting mixture was stirred at −70° C. for 2 h. The reaction mixture was quenched by slowly adding into sat. aq. NH₄Cl (100 mL), and extracted with EtOAc (50 mL*2). The combined organic phase was washed with brine (100 mL), dried over anhydrous sodium sulfate, filtered and concentrated under vacuum. The residue was purified by column chromatography (SiO₂, Petroleum ether/Ethyl acetate=100/0 to 20/1) to give 1-(3-vinyl-2-thienyl)pent-4-en-2-one (8) (4.5 g, 23.4 mmol, 98.9% yield) as a yellow oil. ¹H NMR (400 MHz, CDCl₃) δ 7.24-7.20 (m, 1H), 7.20-7.16 (m, 1H), 6.63 (dd, J=10.8, 17.6 Hz, 1H), 5.96-5.84 (m, 1H), 5.58 (dd, J=1.2, 17.6 Hz, 1H), 5.28 (dd, J=0.8, 10.8 Hz, 1H), 5.24-5.10 (m, 2H), 3.91 (s, 2H), 3.23 (d, J=7.2 Hz, 2H).

### Procedure for the preparation of 6,8-dihydro-7H-cyclohepta[b]thiophen-7-one (9)

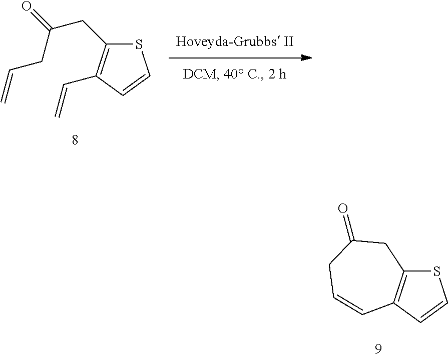

To the solution of 1-(3-vinyl-2-thienyl)pent-4-en-2-one (8) (4.5 g, 23.4 mmol, 1 eq) in DCM (150 mL) was added [1,3-bis(2,4,6-trimethylphenyl)imidazolidin-2-ylidene]-dichloro-[(2-isopropoxyphenyl)methylene]ruthenium (733 mg, 1.17 mmol, 0.05 eq). The resulting mixture was stirred at 40° C. for 2 h. The reaction mixture was concentrated under vacuum. The residue was purified by flash silica gel chromatography (ISCO®; 20 g SepaFlash® Silica Flash Column, eluent 0~5% Ethyl acetate/Petroleum ether) to give 6,8-dihydrocyclohepta [b]thiophen-7-one (9) (2.2 g, 13.4 mmol, 57.2% yield) as a yellow oil. ¹H NMR (400 MHz, CDCl₃) δ 7.08 (d, J=5.2 Hz, 1H), 6.89 (d, J=5.2 Hz, 1H), 6.73 (d, J=10.4 Hz, 1H), 5.77 (td, J=6.0, 10.4 Hz, 1H), 3.65 (s, 2H), 3.10 (d, J=6.0 Hz, 2H).

### Procedure for the preparation of N-methyl-7,8-dihydro-6H-cyclohepta[b]thiophen-7-amine hydrochloride (24)

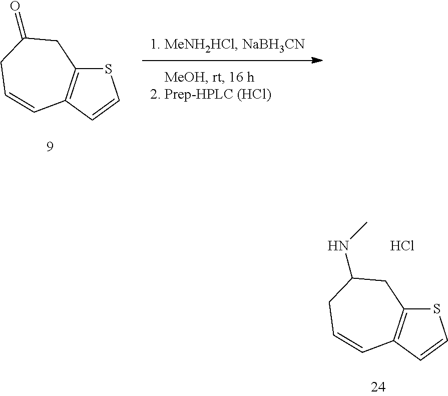

To the mixture of 6,8-dihydrocyclohepta[b]thiophen-7-one (9) (150 mg, 913 μmol, 1 eq) and methylamine hydrochloride (246 mg, 3.65 mmol, 4 eq) in MeOH (5 mL) was added NaBH₃CN (344 mg, 5.48 mmol, 6 eq), and the resulting mixture was stirred at 15° C. for 16 h. The reaction mixture was poured into water (30 mL) and extracted with EtOAc (15 mL*2). The combined organic phase was washed with brine (20 mL), dried over anhydrous sodium sulfate, filtered, and concentrated under vacuum. The residue was purified by prep-HPLC (column: 3_Phenomenex Luna C18 75*30 mm*3 μm; mobile phase: [water (HCl)-ACN]; B %: 0%-16%, 8 min) to give N-methyl-7,8-dihydro-6H-cyclohepta[b]thiophen-7-amine hydrochloride salt (24) (66.80 mg, 306 μmol, 33.6% yield) as an off-white solid. ¹H NMR (400 MHz, MeOD-d₄) δ 7.23 (d, J=5.2 Hz, 1H), 6.93 (d, J=5.2 Hz, 1H), 6.57 (td, J=2.0, 11.6 Hz, 1H), 5.71 (td, J=5.2, 11.6 Hz, 1H), 3.81-3.73 (m, 1H), 3.47-3.36 (m, 2H), 2.92-2.82 (m, 1H), 2.80-2.74 (m, 1H), 2.73 (s, 3H). LCMS: m/z [M+H]⁺=180.2.

### Procedure for the preparation of N-methyl-5,6,7,8-tetrahydro-4H-cyclohepta[b]thiophen-7-amine hydrochloride, enantiomer 1 (25) and enantiomer 2 (26)

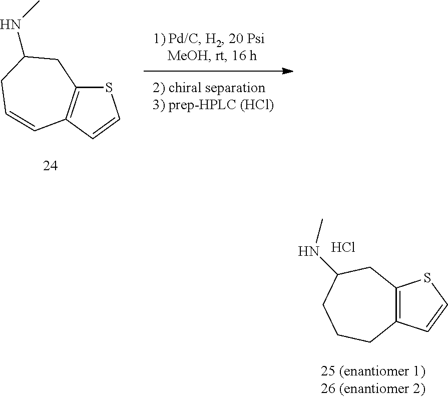

To a solution of N-methyl-7,8-dihydro-6H-cyclohepta[b]thiophen-7-amine (24) (0.5 g, 2.79 mmol, 1 eq) in MeOH (25 mL) was added 10% Pd/C (0.5 g) under N₂ atmosphere. The suspension was degassed and purged with H₂ 3 times. The mixture was stirred under H₂ (20 Psi) at 20-25° C. for 16 h. The reaction mixture was filtered, and the filtrate was concentrated under vacuum to give a racemate, which was separated by prep-Chiral-SFC (column: DAICEL CHIRALPAK AY-H (250 mm*30 mm, 10 μm); mobile phase: [ACN/EtOH (0.1% NH₃H₂O)]; B %: 20%-20%, 11.2 min). The two enantiomers were further subjected to prep-HPLC (column: 3_Phenomenex Luna C18 75*30 mm*3 μm; mobile phase: [water (HCl)-ACN]; B %: 0%-23%, 8 min) to convert to the hydrochloride salts.

Enantiomer 1 (25): 90.12 mg, 410.09 μmol, 14.30% yield, HCl salt, white solid. ¹H NMR (400 MHz, MeOD-d₄) δ 7.09 (d, J=5.2 Hz, 1H), 6.84 (d, J=5.2 Hz, 1H), 3.30-3.23 (m, 2H), 3.18-3.10 (m, 1H), 2.88-2.80 (m, 1H), 2.78-2.69 (m, 4H), 2.34-2.24 (m, 1H), 2.10-1.99 (m, 1H), 1.94-1.83 (m, 1H), 1.72-1.61 (m, 1H). LCMS: m/z [M+H]⁺=182.2.

Enantiomer 2 (26): 122.48 mg, 535.57 μmol, 18.67% yield, HCl salt, white solid. ¹H NMR (400 MHz, MeOD-d₄) δ 7.09 (d, J=5.2 Hz, 1H), 6.84 (d, J=4.8 Hz, 1H), 3.30-3.23 (m, 2H), 3.19-3.11 (m, 1H), 2.92-2.80 (m, 1H), 2.79-2.66 (m, 4H), 2.35-2.24 (m, 1H), 2.10-1.98 (m, 1H), 1.94-1.83 (m, 1H), 1.72-1.60 (m, 1H). LCMS: m/z [M+H]⁺=182.2.

### Synthetic Scheme for the preparation of N, 2-dimethyl-4,5,6,7-tetrahydrobenzothiophen-6-amine hydrochloride, enantiomer 1 (28) and enantiomer 2 (29)

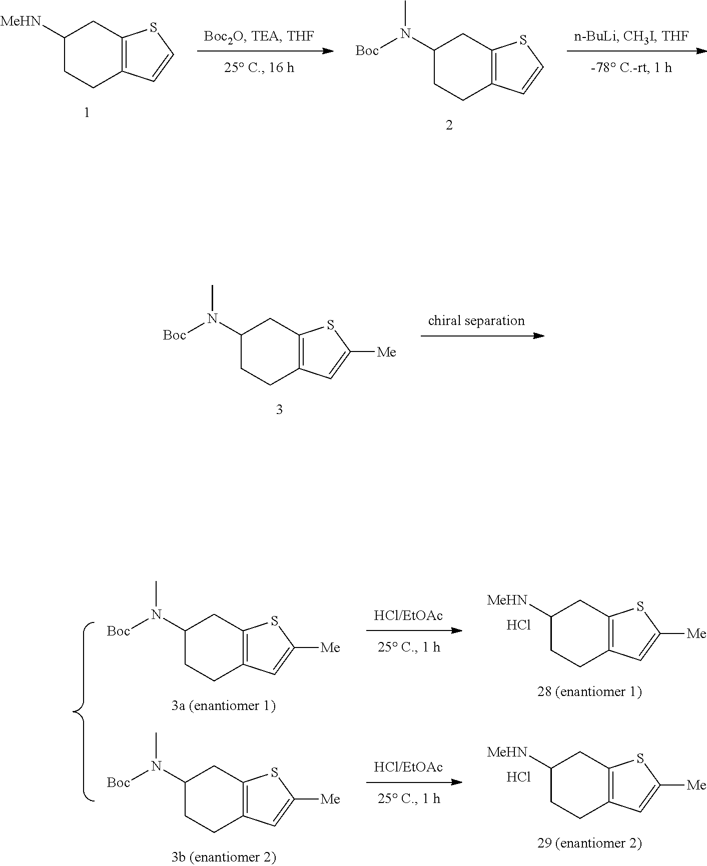

### Procedure for the preparation of tert-butyl N-methyl-N-(4,5,6,7-tetrahydrobenzothiophen-6-yl) carbamate (2)

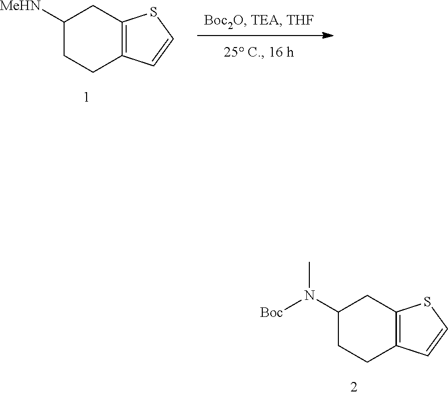 [SMILES: CNC1CCc2ccsc2C1 ; CN(C(=O)OC(C)(C)C)C1CCc2ccsc2C1]

To a solution of (Boc)₂O (2.22 g, 10.16 mmol, 2.34 mL, 2 eq) in THF (10 mL) was added N-methyl-4,5,6,7-tetrahydrobenzothiophen-6-amine (1) (850 mg, 5.08 mmol, 1 eq) and TEA (1.03 g, 10.16 mmol, 1.41 mL, 2 eq), and the resulting mixture was stirred at 25° C. for 16 hr. The mixture was poured into H₂O (20 mL) and extracted with EtOAc (20 mL*3). The combined organic layer was dried over anhydrous Na₂SO₄, filtered, and concentrated in vacuo. The residue was purified by flash silica gel chromatography (ISCO®; 12 g SepaFlash® Silica Flash Column, eluent 0~20% Ethyl acetate/Petroleum ether) to afford tert-butyl N-methyl-N-(4,5,6,7-tetrahydrobenzothiophen-6-yl) carbamate (2) (900 mg, 3.37 mmol, 66.2% yield) as a white solid. ¹H NMR (400 MHz, CHLOROFORM-d) δ 7.08 (d, J=4.9 Hz, 1H), 6.75 (d, J=5.0 Hz, 1H), 4.62-4.21 (m, 1H), 3.03-2.61 (m, 7H), 2.00-1.80 (m, 2H), 1.48 (s, 9H).

### Procedure for the preparation of tert-butyl N-methyl-N-(2-methyl-4,5,6,7-tetrahydro benzo thiophen-6-yl) carbamate (3a) and (3b)

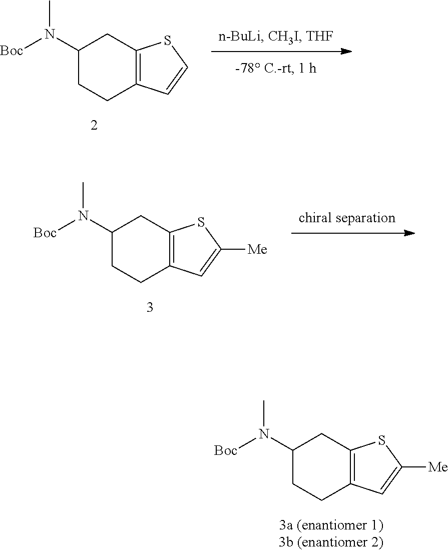 [SMILES: CN(C(=O)OC(C)(C)C)C1CCc2ccsc2C1 ; Cc1cc2c(s1)CC(N(C)C(=O)OC(C)(C)C)CC2 ; Cc1cc2c(s1)CC(N(C)C(=O)OC(C)(C)C)CC2]

To a solution of tert-butyl N-methyl-N-(4,5,6,7-tetrahydrobenzothiophen-6-yl) carbamate (2) (900 mg, 3.37 mmol, 1 eq) in THF (10 mL) was added drop-wise n-BuLi (2.5 M in hexanes, 1.62 mL, 2.43 mmol, 1.2 eq) at −78° C. under N₂ atmosphere. The resulting mixture was stirred at this temperature for 0.5 hr before being treated with MeI (717 mg, 5.05 mmol, 314 μL, 1.5 eq) drop-wise. The reaction mixture was warmed to 25° C. and stirred for 1.5 hr. The mixture was quenched with H₂O (30 mL) and extracted with EtOAc (30 mL*3). The combined organic layer was dried over anhydrous Na₂SO₄, filtered and concentrated in vacuo. The residue was purified by prep-HPLC (column: Waters Xbridge C18 150*50 mm*10 μm; mobile phase: [water (NH₄HCO₃)-ACN]; B %: 57%-87%, 10 min) to give tert-butyl N-methyl-N-(2-methyl-4, 5,6,7-tetrahydrobenzothiophen-6-yl)carbamate (3) as a racemate, which was further separated by prep-Chiral-SFC (column: DAICEL CHIRALPAK AD (250 mm*30 mm, 10 μm); mobile phase: [0.1% NH₃H₂O in MEOH]; B %: 15%-15%, 4.8 min) to successively afford enantiomer 1 (3a) (250 mg, 888.37 μmol, 26.39% yield) and enantiomer 2 (3b) (250 mg, 888.37 μmol, 26.39% yield), both as yellow solids. LCMS: m/z [M+H-tBu]⁺=226.2.

### Procedure for the preparation of N, 2-dimethyl-4,5,6,7-tetrahydrobenzothiophen-6-amine hydrochoride enantiomer 1 (28)

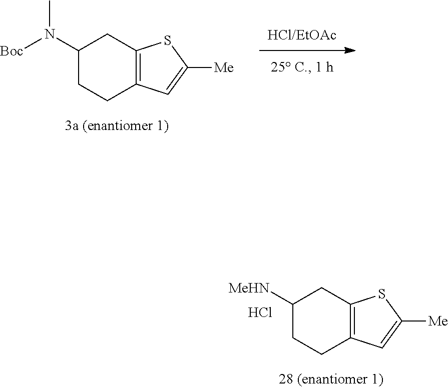 [SMILES: Cc1cc2c(s1)CC(N(C)C(=O)OC(C)(C)C)CC2 ; Cl ; CNC1CCc2cc(C)sc2C1]

To a solution of tert-butyl N-methyl-N-(2-methyl-4,5,6,7-tetrahydrobenzothiophen-6-yl) carbamate (3a) (250 mg, 888.37 μmol, 1 eq) in EtOAc (2 mL) was added HCl/EtOAc (4 M, 2 mL), and the resulting mixture was stirred at 25° C. for 1 hr. The precipitate was collected by filtration, washed with EtOAc, and dried in vacuo to afford N, 2-dimethyl-4,5,6,7-tetrahydrobenzothiophen-6-amine hydrochloride enantiomer 1 (28) (136.33 mg, 626.06 μmol, 70.47% yield) as a white solid. ¹H NMR (400 MHz, METHANOL-d₄) δ ppm 6.46 (s, 1H), 3.46-3.58 (m, 1H), 3.21 (dd, J=16.02, 4.40 Hz, 1H), 2.63-2.84 (m, 6H), 2.39 (s, 3H), 2.18-2.29, (m, 1H), 1.84-1.97 (m, 1H). LCMS: m/z [M+H]⁺=182.1.

### Procedure for the preparation of N, 2-dimethyl-4,5,6,7-tetrahydrobenzothiophen-6-amine hydrochloride enantiomer 2 (29)

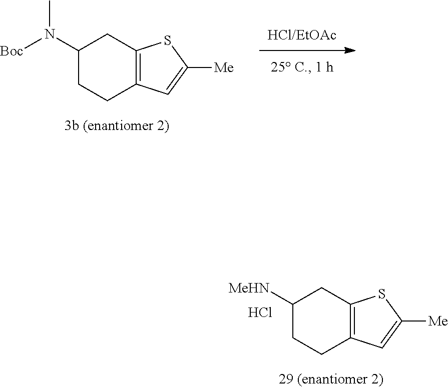 [SMILES: Cc1cc2c(s1)CC(N(C)C(=O)OC(C)(C)C)CC2 ; Cl ; CNC1CCc2cc(C)sc2C1]

To a solution of tert-butyl N-methyl-N-(2-methyl-4,5,6,7-tetrahydrobenzothiophen-6-yl) carbamate enantiomer 2 (3b) (250 mg, 888 μmol, 1 eq) in EtOAc (2 mL) was added HCl/EtOAc (4 M, 2 mL). The mixture was stirred at 25° C. for 1 hr. The precipitate was collected by filtration, washed with EtOAc, and dried in vacuo to afford N, 2-dimethyl-4,5,6,7-tetrahydrobenzothiophen-6-amine hydrochloride salt enantiomer 2 (29) (183.40 mg, 842.22 μmol, 94.80% yield) as a white solid. ¹H NMR (400 MHz, METHANOL-d₄) δ ppm 6.46 (s, 1H), 3.46-3.57 (m, 1H), 3.21 (dd, J=15.53, 4.89 Hz, 1H), 2.65-2.84 (m, 6H), 2.38 (m, 3H), 2.18-2.29 (m, 1H), 1.83-1.96 (m, 1H). LCMS: m/z [M+H]⁺=182.1.

### Synthetic scheme for the preparation of N, 1,3-trimethyl-4,5,6,7-tetrahydro-2-benzothiophen-5-amine hydrochloride salt enantiomer 1 (30) and enantiomer 2 (31)

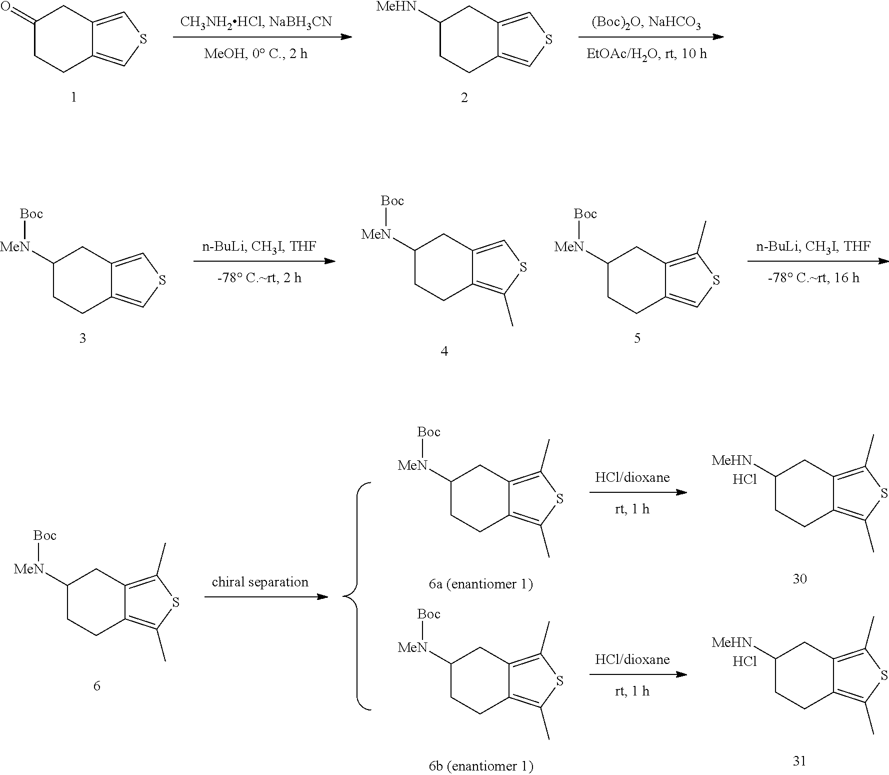

### Procedure for the preparation of N-methyl-4,5,6,7-tetrahydro-2-benzothiophen-5-amine (2)

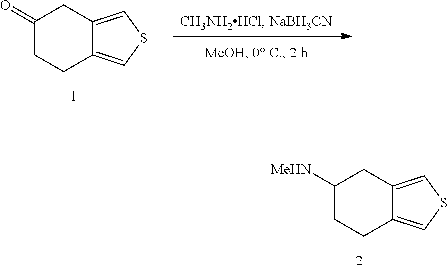 [SMILES: O=C1CCc2cscc2C1 ; CNC1CCc2cscc2C1]

To a mixture of 6,7-dihydro-4H-2-benzothiophen-5-one (1) (2 g, 13.14 mmol, 1 eq) and methylamine hydrochloride (1.77 g, 26.28 mmol, 2 eq, HCl) in MeOH (30 mL) was added NaBH₃CN (4.95 g, 78.84 mmol, 6 eq) batch-wise at 0° C., and the resulting mixture was stirred at 0° C. for 2 hr. The reaction mixture was quenched with aq. Na₂CO₃ (50 mL) and extracted with EtOAc (30 mL*2). The combined organic layers were washed with brine, dried over anhydrous sodium sulfate, filtered and concentrated under reduced pressure to give N-methyl-4,5,6,7-tetrahydro-2-benzothiophen-5-amine (2) (2.2 g, crude) as a brown oil.

### Procedure for the preparation of tert-butyl N-methyl-N-(4,5,6,7-tetrahydro-2-benzothiophen-5-yl) carbamate (3)

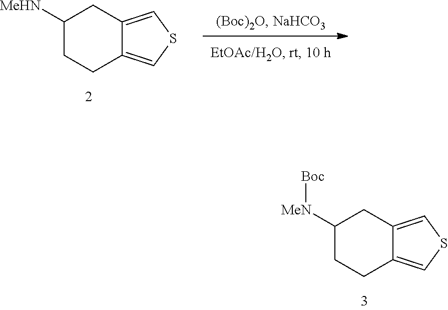 [SMILES: CNC1CCc2cscc2C1 ; CN(C(=O)OC(C)(C)C)C1CCc2cscc2C1]

To a solution of N-methyl-4,5,6,7-tetrahydro-2-benzothiophen-5-amine (2) (2 g, crude) in EtOAc (40 mL) and H₂O (20 mL) was added NaHCO₃ (3.01 g, 35.87 mmol, 1.40 mL). Boc₂O (5.22 g, 23.91 mmol, 5.49 mL) was then added, and the mixture was stirred at 20° C. for 10 hr. The reaction mixture was diluted with H₂O (20 mL) and extracted with EtOAc (20 mL*2). The combined organic layers were washed with brine, dried over anhydrous sodium sulfate, filtered, and concentrated under reduced pressure. The residue was purified by flash silica gel chromatography (ISCO®; 20 g SepaFlash® Silica Flash Column, eluent 0~15% Ethyl acetate/Petroleum ether) to give tert-butyl N-methyl-N-(4,5,6,7-tetrahydro-2-benzothiophen-5-yl) carbamate (3) (1.7 g, 6.10 mmol, 46.4% yield over two steps) as a colorless oil. ¹H NMR (400 MHz, CHLOROFORM-d) δ 6.89 (s, 2H), 4.54-4.13 (m, 1H), 3.01-2.87 (m, 2H), 2.85-2.67 (m, 5H), 1.94-1.76 (m, 2H), 1.48 (s, 9H). LCMS: m/z [M+H-tBu]⁺=212.0.

### Procedure for the preparation of tert-butyl methyl(1-methyl-4,5,6,7-tetrahydrobenzo [c]thiophen-5-yl)carbamate (4) and tert-butyl methyl(3-methyl-4,5,6,7-tetrahydrobenzo [c]thiophen-5-yl)carbamate (5)

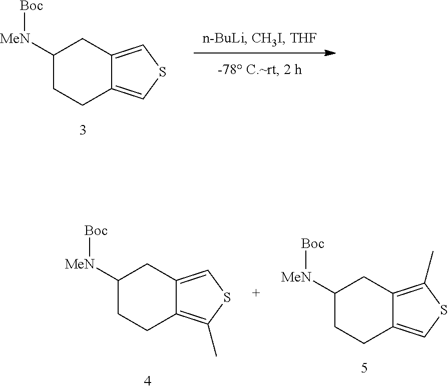 [SMILES: CN(C(=O)OC(C)(C)C)C1CCc2cscc2C1 ; Cc1scc2c1CCC(N(C)C(=O)OC(C)(C)C)C2 ; Cc1scc2c1CC(N(C)C(=O)OC(C)(C)C)CC2]

To a solution of tert-butyl N-methyl-N-(4,5,6,7-tetrahydro-2-benzothiophen-5-yl)carbamate (3) (1.6 g, 5.98 mmol, 1 eq) in THF (15 mL) was added drop-wise n-BuLi (2.5 M in hexanes, 2.87 mL, 1.2 eq) at −78° C. under N₂ atmosphere, and the resulting mixture was stirred at this temperature for 0.5 hr. A solution of CH₃I (1.02 g, 7.18 mmol, 447 μL, 1.2 eq) in THF (5 mL) was then added dropwise at −78° C., and the resulting mixture was warmed to 25° C. and stirred for 1.5 hr. The mixture was quenched with aq. sat. NH₄Cl solution (20 mL), and extracted with EtOAc (20 mL*3). The combined organic was dried over anhydrous Na₂SO₄, filtered and concentrated in vacuo. The residue was purified by flash silica gel chromatography (ISCO®; 24 g SepaFlash® Silica Flash Column, eluent 0~20% Ethyl acetate/Petroleum ether) to give a mixture of tert-butyl methyl(1-methyl-4,5,6,7-tetrahydrobenzo[c]thiophen-5-yl)carbamate (4) and tert-butyl methyl(3-methyl-4,5,6,7-tetrahydrobenzo[c]thiophen-5-yl)carbamate (5) (1.6 g, 5.69 mmol, 95.02% yield) as a yellow oil. LCMS: m/z [M+H-tBu]⁺=226.4.

### Procedure for the preparation of tert-butyl N-(1,3-dimethyl-4,5,6,7-tetrahydro-2-benzothiophen-5-yl)-N-methyl-carbamate enantiomer 1 (6a) and enantiomer 2 (6b)

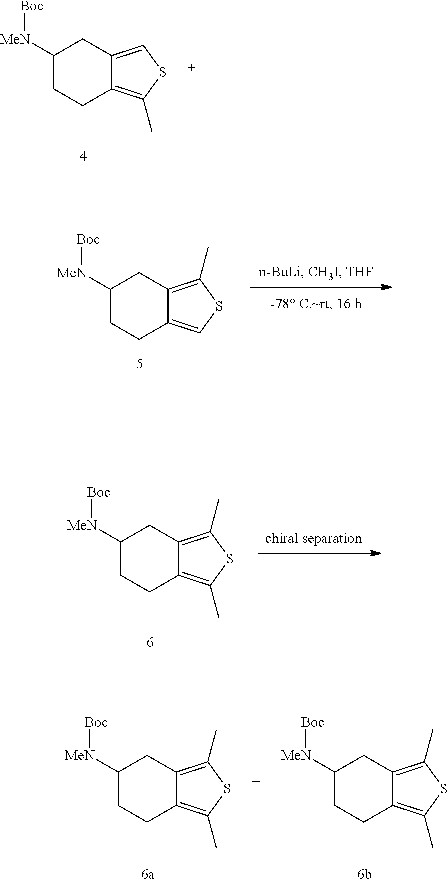 [SMILES: Cc1scc2c1CCC(N(C)C(=O)OC(C)(C)C)C2 ; Cc1scc2c1CC(N(C)C(=O)OC(C)(C)C)CC2 ; Cc1sc(C)c2c1CCC(N(C)C(=O)OC(C)(C)C)C2 ; Cc1sc(C)c2c1CCC(N(C)C(=O)OC(C)(C)C)C2 ; Cc1sc(C)c2c1CCC(N(C)C(=O)OC(C)(C)C)C2]

To a solution of the mixture of tert-butyl methyl(1-methyl-4,5,6,7-tetrahydrobenzo[c]thiophen-5-yl)carbamate (4) and tert-butyl methyl(3-methyl-4,5,6,7-tetrahydrobenzo[c]thiophen-5-yl)carbamate (5) (1.4 g, 4.97 mmol, 1 eq) in THF (20 mL) was added drop-wise n-BuLi (2.5 M in hexanes, 2.39 mL, 5.98 mmol, 1.2 eq) at −78° C. under N₂ atmosphere, and the resulting mixture was stirred at this temperature for 0.5 hr before being treated with CH₃I (1.06 g, 7.46 mmol, 464 μL, 1.5 eq) drop-wise. The reaction mixture was warmed to 25° C. and stirred for 16 hr. The reaction mixture was quenched with sat.NH₄Cl solution (30 mL), and extracted with EtOAc (30 mL*3). The combined organic layer was dried over anhydrous Na₂SO₄, filtered and concentrated in vacuo. The residue was purified by prep-HPLC (column: Waters Xbridge C18 150*50 mm*10 μm; mobile phase: [water (NH₄HCO₃)-ACN]; B %: 55%-85%, 10 min) to give the racemic tert-butyl N-(1,3-dimethyl-4,5,6,7-tetrahydro-2-benzothiophen-5-yl)-N-methyl-carbamate (6) (370 mg, 1.25 mmol, 25.17% yield), which was further separated by prep-Chiral-SFC (column: DAICEL CHIRALPAK IG 250 mm*30 mm*5μm; mobile phase: [0.1% NH₃H₂O MEOH]; B %: 15%-15%, 1.55 min) to successively give enantiomer 1 (6a) (130 mg, 440.02 μmol, 9.3% yield) and enantiomer 2 (6b) (150 mg, 507.72 μmol, 10.8% yield), both as yellow oil.

### Procedure for the preparation of N, 1,3-trimethyl-4,5,6,7-tetrahydro-2-benzothiophen-5-amine hydrochloride enantiomer 1 (30)

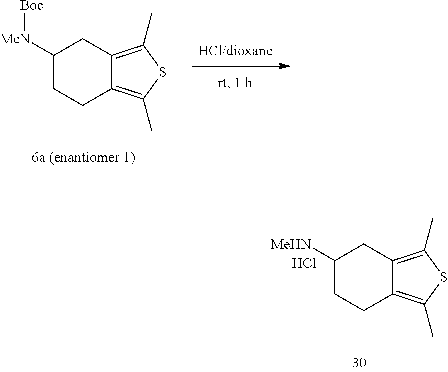 [SMILES: Cc1sc(C)c2c1CCC(N(C)C(=O)OC(C)(C)C)C2 ; Cl ; CNC1CCc2c(C)sc(C)c2C1]

To a solution of tert-butyl N-(1,3-dimethyl-4,5,6,7-tetrahydro-2-benzothiophen-5-yl)-N-methyl-carbamate enantiomer 1 (6a) (130 mg, 440.02 μmol, 1 eq) in EtOAc (4 mL) was added HCl/dioxane (4 M, 4 mL). The mixture was stirred at 25° C. for 1 hr. The reaction mixture was concentrated to dryness under reduced pressure. The crude product was purified by trituration with EtOAc to give N, 1,3-trimethyl-4,5,6,7-tetrahydro-2-benzothiophen-5-amine hydrochloride enantiomer 1 (30) (77 mg, 328.88 μmol, 74.74% yield) as a white solid. ¹H NMR (400 MHz, METHANOL-d₄) δ 3.40 (ddt, J=3.0, 5.3, 10.1 Hz, 1H), 3.10 (dd, J=5.0, 15.5 Hz, 1H), 2.84-2.72 (m, 4H), 2.63-2.48 (m, 2H), 2.27-2.18 (m, 7H), 1.88-1.74 (m, 1H). LCMS: m/z [M+H]⁺=196.1.

### Procedure for the preparation of N, 1,3-trimethyl-4,5,6,7-tetrahydro-2-benzothiophen-5-amine hydrochloride enantiomer 2 (31)

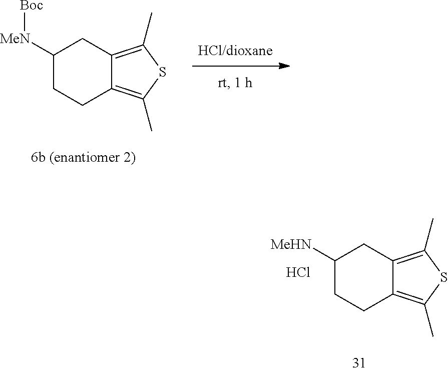 [SMILES: Cc1sc(C)c2c1CCC(N(C)C(=O)OC(C)(C)C)C2 ; Cl ; CNC1CCc2c(C)sc(C)c2C1]

To a solution of tert-butyl N-(1,3-dimethyl-4,5,6,7-tetrahydro-2-benzothiophen-5-yl)-N-methyl-carbamate enantiomer 2 (6b) (150 mg, 507.72 μmol, 1 eq) in EtOAc (5 mL) was added HCl/dioxane (4 M, 3 mL). The mixture was stirred at 25° C. for 1 hr. The reaction mixture was concentrated to dryness under reduced pressure. The crude product was purified by trituration with EtOAc (2 mL) to give N, 1,3-trimethyl-4,5,6,7-tetrahydro-2-benzothiophen-5-amine hydrochloride enantiomer 2 (31) (95 mg, 409.86 μmol, 80.73% yield) as a white solid. ¹H NMR (400 MHz, METHANOL-d₄) δ 3.40 (ddt, J=2.5, 5.3, 10.1 Hz, 1H), 3.09 (dd, J=5.5, 15.5 Hz, 1H), 2.84-2.74 (m, 4H), 2.63-2.47 (m, 2H), 2.27-2.18 (m, 7H), 1.85-1.75 (m, 1H). LCMS: m/z [M+H]⁺=196.1.

### Synthetic scheme for the preparation of N, 1-dimethyl-4,5,6,7-tetrahydro-2-benzothiophen-5-amine hydrochloride enantiomer 1 (32) and enantiomer 2 (33)

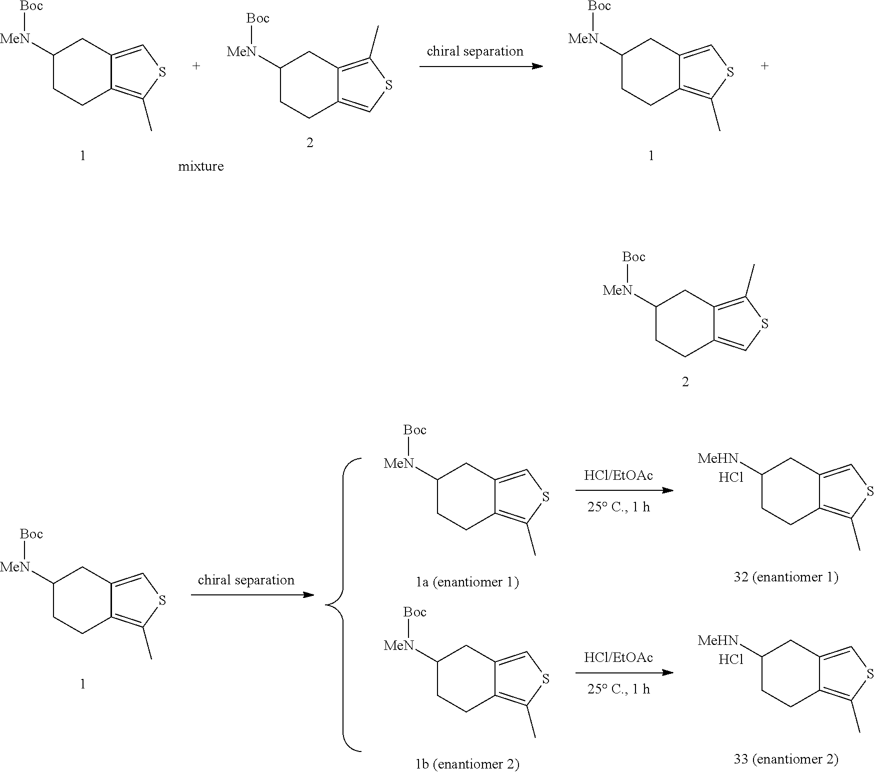

The synthesis of the regioisomeric mixture of tert-butyl methyl(1-methyl-4,5,6,7-tetrahydrobenzo[c]thiophen-5-yl)carbamate (1) and tert-butyl methyl(3-methyl-4,5,6,7-tetrahydrobenzo[c]thiophen-5-yl)carbamate (2) was described for the synthesis of compounds 30 and 31. This mixture was separated by prep-Chiral-SFC (column: DAICEL CHIRALPAK AD-H (250 mm*30 mm, 5 μm); mobile phase: [0.1% NH₃H₂O MEOH]; B %: 15%-15%, 4.5 min).

Racemic tert-butyl N-methyl-N-(1-methyl-4,5,6,7-tetrahydro-2-benzothiophen-5-yl)carbamate (1) (250 mg) was further separated by prep-Chiral-SFC (column: DAICEL CHIRALPAK IC (250 mm*30 mm, 5 μm); mobile phase: [0.1% NH₃H₂O IPA]; B %: 20%-20%, 2.6 min) to successively give enantiomer 1 (1a) (100 mg) and enantiomer 2 (1b) (120 mg).

### Procedure for the preparation of N, 1-dimethyl-4,5,6,7-tetrahydro-2-benzothiophen-5-amine hydrochloride enantiomer 1 (32)

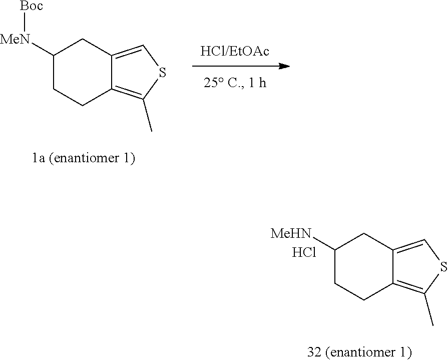 [SMILES: Cc1scc2c1CCC(N(C)C(=O)OC(C)(C)C)C2 ; Cl ; CNC1CCc2c(csc2C)C1]

A mixture of tert-butyl N-methyl-N-(1-methyl-4,5,6,7-tetrahydro-2-benzothiophen-5-yl)carbamate enantiomer 1 (1a) (100 mg, 355 μmol, 1 eq) in 2 N HCl/EtOAc (4 mL) was stirred at room temperature for 1 h. The reaction mixture was concentrated to dryness under reduced pressure. The crude product was purified by trituration with EtOAc to give N, 1-dimethyl-4,5,6,7-tetrahydro-2-benzothiophen-5-amine hydrochloride salt enantiomer 1 (32) (60.78 mg, 279 μmol, 78.6% yield) as a white solid. ¹H NMR (400 MHz, CDCl₃) δ (ppm)=9.80 (br s, 2H), 6.72 (s, 1H), 3.41-3.21 (m, 2H), 3.01 (dd, J=11.2, 14.0 Hz, 1H), 2.92-2.82 (m, 1H), 2.75 (t, J=5.2 Hz, 3H), 2.61-2.44 (m, 2H), 2.31 (s, 3H), 2.16-1.98 (m, 1H). LCMS: m/z [M+H]⁺=182.1.

### Procedure for the preparation of N, 1-dimethyl-4,5,6,7-tetrahydro-2-benzothiophen-5-amine hydrochloride enantiomer 2 (33)

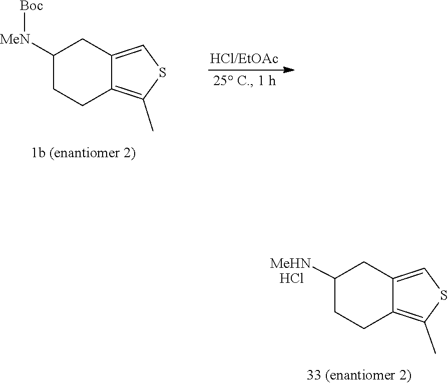 [SMILES: Cc1scc2c1CCC(N(C)C(=O)OC(C)(C)C)C2 ; Cl ; CNC1CCc2c(csc2C)C1]

A mixture of tert-butyl N-methyl-N-(1-methyl-4,5,6,7-tetrahydro-2-benzothiophen-5-yl)carbamate enantiomer 2 (1b) (120 mg, 426 μmol, 1 eq) in 2 N HCl/EtOAc (4 mL) was stirred at room temperature for 1 h. The reaction mixture was concentrated to dryness under reduced pressure. The crude product was purified by trituration with EtOAc to give N, 1-dimethyl-4,5,6,7-tetrahydro-2-benzothiophen-5-amine hydrochloride salt enantiomer 2 (33) (77.54 mg, 356 μmol, 83.5% yield) as a white solid. ¹H NMR (400 MHz, CDCl₃) δ (ppm)=9.80 (br s, 2H), 6.72 (s, 1H), 3.40-3.23 (m, 2H), 3.01 (dd, J=11.2, 14.0 Hz, 1H), 2.93-2.82 (m, 1H), 2.75 (t, J=5.2 Hz, 3H), 2.59-2.44 (m, 2H), 2.31 (s, 3H), 2.15-2.03 (m, 1H). LCMS: m/z [M+H]⁺=182.1.

### Synthetic scheme for the preparation of N,N, 2-trimethyl-4,5,6,7-tetrahydrobenzo thiophen-6-amine hydrochloride enantiomer 1 (34) and enantiomer 2 (35)

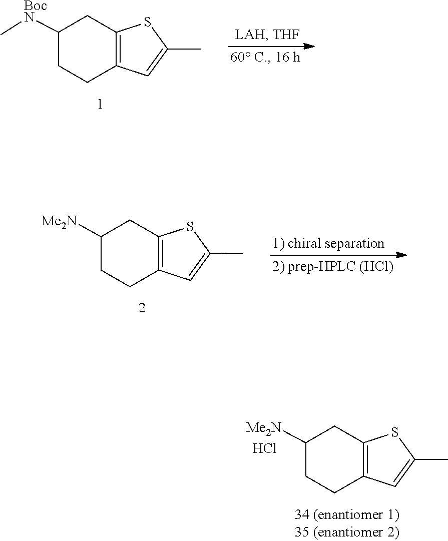

The synthesis of intermediate 1, tert-butyl N-methyl-N-(2-methyl-4,5,6,7-tetrahydrobenzothiophen-6-yl) carbamate, is as described in the synthesis of compounds 42 and 43.

### Procedure for the preparation of N,N, 2-trimethyl-4,5,6,7-tetrahydrobenzothiophen-6-amine hydrochloride enantiomer 1 (34) and enantiomer 2 (35)

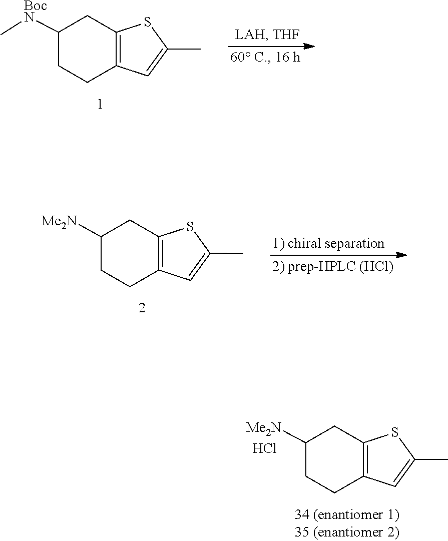

To a solution of tert-butyl N-methyl-N-(2-methyl-4,5,6,7-tetrahydrobenzothiophen-6-yl) carbamate (1) (1.50 g, 5.33 mmol, 1 eq) in THF (20 mL) was added LAH (404 mg, 10.66 mmol, 2 eq) batch-wise at 0° C., and the resulting mixture was stirred at 60° C. for 16 hr. The reaction mixture was cooled to 0° C. and quenched by slowly adding Na₂SO₄·10H₂O. The quenched reaction mixture was stirred for 30 min before being filtered, and the filtrate was concentrated under reduced pressure. The residue was purified by prep-HPLC (column: Waters Xbridge C18 150*50 mm*10 μm; mobile phase: [water (NH₄HCO₃)-ACN]; B %: 22%-52%, 10 min) to give the racemic product (2), which was further separated by prep-Chiral-SFC (column: DAICEL CHIRALPAK AY (250 mm*30 mm, 20 μm); mobile phase: [0.1% NH₃H₂O in ETOH]; B %: 30%-30%, 4.4 min) to successively give two enantiomers.

Enantiomer 1 was subjected to prep-HPLC (column: 3_Phenomenex Luna C18 75*30 mm*3 μm; mobile phase: [water (HCl)-ACN]; B %: 6%-26%, 8 min) and lyophilized to afford N,N, 2-trimethyl-4,5,6,7-tetrahydrobenzothiophen-6-amine hydrochloride salt enantiomer 1 (34) (130.76 mg, 669.45 μmol, 12.56% yield) as an off-white gum. ¹H NMR (400 MHz, METHANOL-d₄) δ ppm 6.45 (s, 1H), 3.71-3.61 (m, 1H), 3.17 (dd, J=5.20, 5.07 Hz, 1H), 3.00-2.92 (m, 7H), 2.86-2.78 (m, 1H), 2.74-2.63 (m, 1H), 2.38 (s, 3H), 2.32-2.23 (m, 1H), 1.93 (qd, J=11.76, 5.75 Hz, 1H). LCMS: m/z [M+H]⁺=196.5.

Enantiomer 2 was subjected to prep-HPLC (column: 3_Phenomenex Luna C18 75*30 mm*3 μm; mobile phase: [water (HCl)-ACN]; B %: 6%-26%, 8 min) and lyophilized to afford N,N, 2-trimethyl-4,5,6,7-tetrahydrobenzothiophen-6-amine hydrochloride salt enantiomer 2 (35) (139.15 mg, 712.41 μmol, 13.37% yield) as an off-white gum. ¹H NMR (400 MHz, METHANOL-d₄) δ ppm 6.45 (s, 1H), 3.59-3.73 (m, 1H), 3.17 (dd, J=15.20, 5.07 Hz, 1H), 3.00-2.92 (d, J=4.13 Hz, 7H), 2.86-2.76 (m, 1H), 2.73-2.62 (m, 1H), 2.38 (s, 3H), 2.32-2.28 (m, 1H), 1.93 (qd, J=11.78, 5.69 Hz, 1H). LCMS: m/z [M+H]⁺=196.5.

### Synthetic scheme for the preparation of 6-(methylamino)-4,5,6,7-tetrahydrobenzo thiophene-2-carbonitrile hydrochloride enantiomer 1 (36) and enantiomer 2 (37)

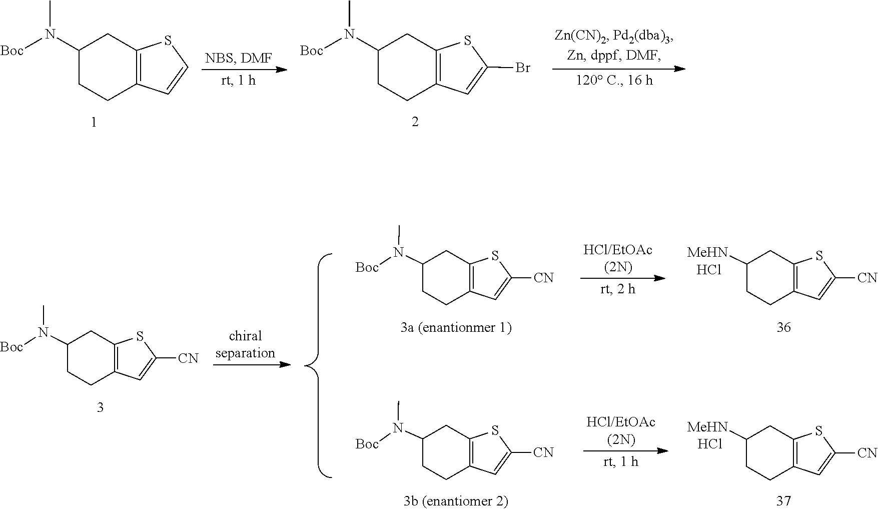

### Procedure for the preparation of tert-butyl N-(2-bromo-4,5,6,7-tetrahydrobenzothiophen-6-yl)-N-methylcarbamate (2)

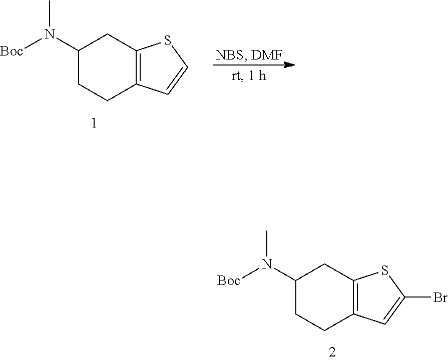

To a solution of tert-butyl N-methyl-N-(4,5,6,7-tetrahydrobenzothiophen-6-yl) carbamate (1) (1.0 g, 3.74 mmol, 1 eq) in DMF (10 mL) was added NBS (732 mg, 4.11 mmol, 1.1 eq) batch-wise, and the resulting mixture was stirred at 25° C. for 1 hr. The mixture was poured into H₂O (30 mL) and extracted with EtOAc (30 mL*3). The combined organic layer was dried over Na₂SO₄, filtered, and concentrated to dryness in vacuo. The residue was purified by flash silica gel chromatography (ISCO®; 12 g SepaFlash® Silica Flash Column, eluent 0~10% Ethyl acetate/Petroleum ether) to afford tert-butyl N-(2-bromo-4,5,6,7-tetrahydrobenzothiophen-6-yl)-N-methylcarbamate (2) (1.2 g, 3.47 mmol, 92.66% yield) as a white solid. ¹H NMR (400 MHz, METHANOL-d₄) δ ppm 6.75 (s, 1H), 4.35-4.20 (m, 1H), 2.88-2.60 (m, 7H), 1.97-1.78 (m, 2H), 1.47 (s, 9H).

### Procedure for the preparation of tert-butyl N-(2-cyano-4,5,6,7-tetrahydrobenzothiophen-6-yl)-N-methyl-carbamate (3)

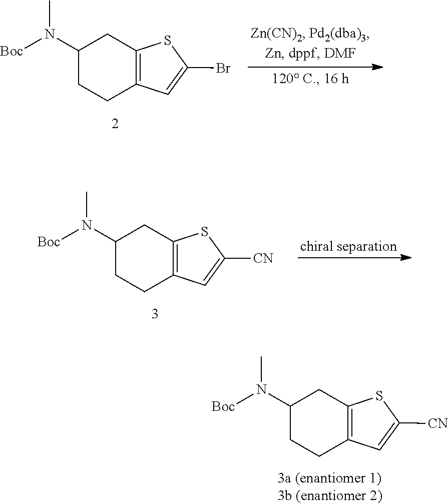 [SMILES: CN(C(=O)OC(C)(C)C)C1CCc2cc(Br)sc2C1 ; CN(C(=O)OC(C)(C)C)C1CCc2cc(C#N)sc2C1 ; CN(C(=O)OC(C)(C)C)C1CCc2cc(C#N)sc2C1]

To a solution of tert-butyl N-(2-bromo-4,5,6,7-tetrahydrobenzothiophen-6-yl)-N-methyl-carbamate (2) (1.1 g, 3.18 mmol, 1 eq) in DMF (15 mL) were added Zn(CN)₂ (895 mg, 7.62 mmol, 2.4 eq), Zn dust (52 mg, 794 μmol, 0.25 eq), Pd₂(dba)₃ (291 mg, 318 μmol, 0.1 eq) and DPPF (352 mg, 635 μmol, 0.2 eq), and the resulting mixture was stirred at 120° C. for 16 h. The reaction mixture was cooled to rt, poured into H₂O (10 mL), and extracted with EtOAc (10 mL*3). The combined organic layers were washed with brine (20 mL*2), dried over Na₂SO₄, filtered and concentrated to dryness in vacuo. The residue was purified by flash silica gel chromatography (ISCO®; 20 g SepaFlash® Silica Flash Column, eluent 0-20% Ethyl acetate/Petroleum ether) to afford tert-butyl N-(2-cyano-4,5,6,7-tetrahydrobenzothiophen-6-yl)-N-methyl-carbamate (3) (720 mg, 2.09 mmol, 65.89% yield) as a yellow oil. LCMS: m/z [M+H-tBu]⁺=237.4.

The racemate was further purified by prep-HPLC (column: Waters Xbridge C18 150*50 mm*10 μm; mobile phase: [water (NH₄HCO₃)-ACN]; B %: 45%-75%, 10 min), and then separated by prep-Chiral-SFC (column: DAICEL CHIRALPAK AD (250 mm*30 mm, 10 μm); mobile phase: [0.1% NH₃H₂O in MeOH]; B %: 30%-30%, 5.4 min) to afford the 1^{st} enantiomer (3a) and the 2^{nd} enantiomer (3b), both as white solids.

### Procedure for the preparation of 6-(methylamino)-4,5,6,7-tetrahydrobenzothiophene-2-carbonitrile hydrochloride enantiomer 1 (36)

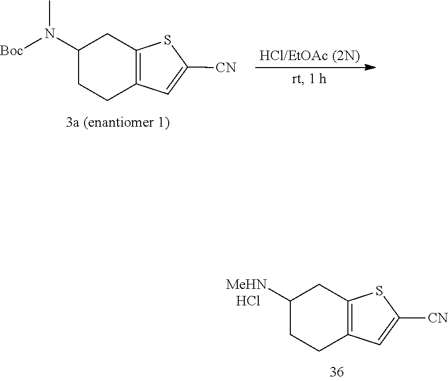 [SMILES: CN(C(=O)OC(C)(C)C)C1CCc2cc(C#N)sc2C1 ; Cl ; CNC1CCc2cc(C#N)sc2C1]

To a solution of tert-butyl N-(2-cyano-4,5,6,7-tetrahydrobenzothiophen-6-yl)-N-methylcarbamate enantiomer 1 (3a) (170 mg, 581 μmol, 1 eq) in EtOAc (5 mL) was added 4 M HCl/EtOAc (5 mL), and the resulting mixture was stirred at room temperature for 1 h. The reaction mixture was concentrated to dryness under reduced pressure. The crude product was purified by trituration with EtOAc to give 6-(methylamino)-4,5,6,7-tetrahydrobenzothiophene-2-carbonitrile hydrochloride salt enantiomer 1 (36) (110.09 mg, 481 μmol, 82.8% yield) as a white solid. ¹H NMR (400 MHz, Methanol-d4) δ (ppm)=7.51 (s, 1H), 3.66-3.56 (m, 1H), 3.40 (dd, J=5.2, 16.8 Hz, 1H), 3.00-2.85 (m, 2H), 2.82-2.73 (m, 4H), 2.38-2.23 (m, 1H), 2.01-1.89 (m, 1H). LCMS: m/z [M+H]⁺=193.0.

### Procedure for the preparation of 6-(methylamino)-4,5,6,7-tetrahydrobenzothiophene-2-carbonitrile hydrochloride enantiomer 2 (37)

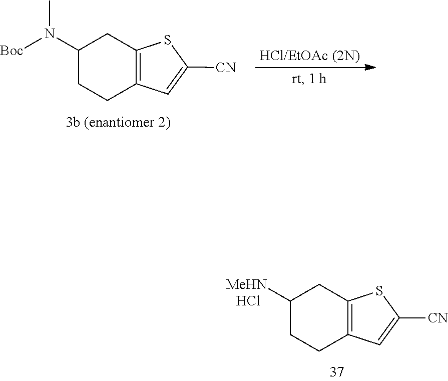 [SMILES: CN(C(=O)OC(C)(C)C)C1CCc2cc(C#N)sc2C1 ; Cl ; CNC1CCc2cc(C#N)sc2C1]

To a solution of tert-butyl N-(2-cyano-4,5,6,7-tetrahydrobenzothiophen-6-yl)-N-methylcarbamate enantiomer 2 (3b) (160 mg, 547 μmol, 1 eq) in EtOAc (5 mL) was added HCl/EtOAc (4 M, 5 mL). The mixture was stirred at room temperature for 1 h. The reaction mixture was concentrated to dryness under reduced pressure. The crude product was purified by trituration with EtOAc to give 6-(methylamino)-4,5,6,7-tetrahydrobenzothiophene-2-carbonitrile hydrochloride salt enantiomer 2 (37) (117.57 mg, 546 μmol, 99.8% yield) as a white solid. ¹H NMR (400 MHz, Methanol-d4) δ (ppm)=7.50 (s, 1H), 3.67-3.56 (m, 1H), 3.40 (dd, J=5.2, 16.8 Hz, 1H), 3.00-2.74 (m, 6H), 2.36-2.26 (m, 1H), 2.01-1.88 (m, 1H). LCMS: m/z [M+H]⁺=193.0.

### Synthetic scheme for the preparation of N-methyl-2-(trifluoromethyl)-4,5,6,7-tetrahydrobenzothiophen-6-amine hydrochloride enantiomer 1 (38) and enantiomer 2 (39)

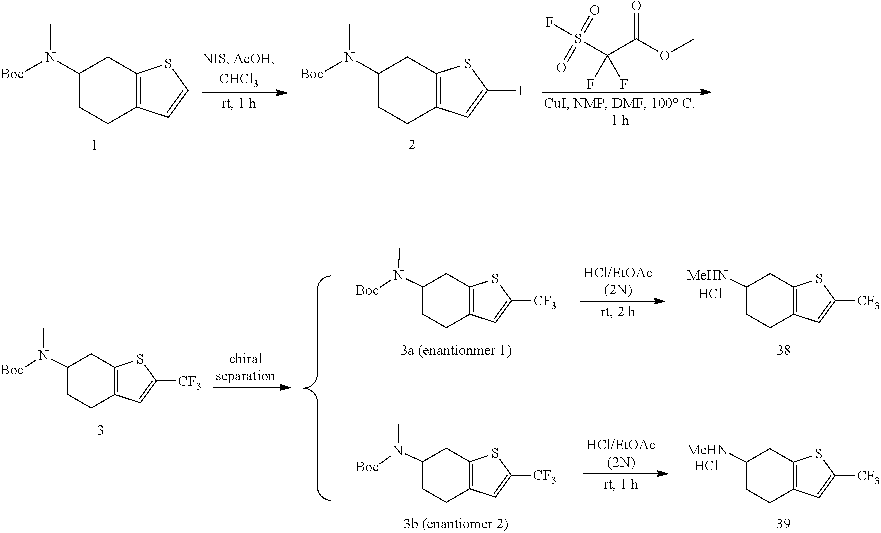

### Procedure for the preparation of tert-butyl N-(2-iodo-4,5,6,7tetrahydrobenzothiophen-6-yl)-N-methyl-carbamate (2)

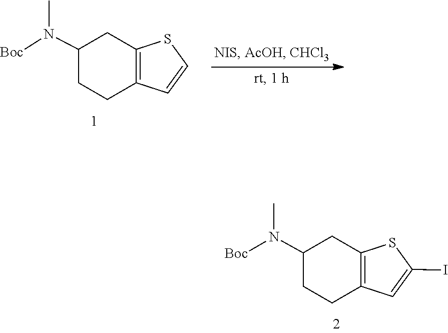 [SMILES: CN(C(=O)OC(C)(C)C)C1CCc2ccsc2C1 ; CN(C(=O)OC(C)(C)C)C1CCc2cc(I)sc2C1]

To a solution of tert-butyl N-methyl-N-(4,5,6,7-tetrahydrobenzothiophen-6-yl)carbamate (1) (1.0 g, 3.74 mmol, 1 eq) in CHCl₃ (5 mL) and AcOH (5 mL) was added NIS (925 mg, 4.11 mmol, 1.1 eq) batch-wise, and the resulting mixture was stirred at 25° C. for 1 hr. The mixture was poured into H₂O (30 mL) and extracted with EtOAc (30 mL*3). The combined organic was washed with sat.NaHCO₃ (30 mL*2), dried over Na₂SO₄, filtered and concentrated in vacuo. The residue was purified by flash silica gel chromatography (ISCO®; 12 g SepaFlash® Silica Flash Column, eluent 0~10% Ethyl acetate/Petroleum ether) to afford tert-butyl N-(2-iodo-4,5,6,7tetrahydrobenzothiophen-6-yl)-N-methylcarbamate (2) (1.4 g, 3.56 mmol, 95.18% yield) as a white solid. ¹HNMR (400 MHz, METHANOL-d₄) δ ppm 6.91 (s, 1H), 4.18-4.39 (m, 1H), 2.62-2.90 (m, 7H), 1.77-2.03 (m, 2H), 1.47 (s, 9H).

### Procedure for the preparation of tert-butyl N-methyl-N-[2-(trifluoromethyl)-4,5,6,7-tetrahydrobenzothiophen-6-yl]carbamate enantiomer 1 (3a) and enantiomer 2 (3b)

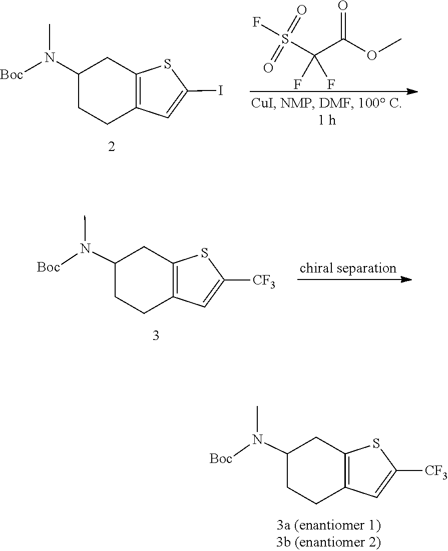 [SMILES: CN(C(=O)OC(C)(C)C)C1CCc2cc(I)sc2C1 ; COC(=O)C(F)(F)S(=O)(=O)F ; CN(C(=O)OC(C)(C)C)C1CCc2cc(C(F)(F)F)sc2C1 ; CN(C(=O)OC(C)(C)C)C1CCc2cc(C(F)(F)F)sc2C1]

To a solution of tert-butyl N-(2-iodo-4,5,6,7-tetrahydrobenzothiophen-6-yl)-N-methyl-carbamate (2) (1.3 g, 3.31 mmol, 1 eq) in DMF (40 mL) was added CuI (692 mg, 3.64 mmol, 1.1 eq) and NMP (1.64 g, 16.5 mmol, 5 eq). The resulting mixture was stirred at room temperature for 10 min before the addition of methyl 2,2-difluoro-2-fluorosulfonyl-acetate (3.18 g, 16.5 mmol, 5 eq), and the stirring was continued at 100° C. for 1 hr. The reaction mixture was cooled to rt, poured into H₂O (200 mL) and extracted with EtOAc (100 mL*3). The combined organic layers were washed with brine (50 mL), dried over anhydrous Na₂SO₄, filtered, and concentrated under reduced pressure. The residue was purified by flash silica gel chromatography (ISCO®; 12 g SepaFlash® Silica Flash Column, eluent 0~20% Ethyl acetate/Petroleum ether) to give the racemic tert-butyl N-methyl-N-[2-(trifluoromethyl)-4,5,6,7-tetrahydrobenzothiophen-6-yl]carbamate (3) (600 mg, 50.3% yield).

The racemate (600 mg) was further separated by prep-Chiral-SFC (column: DAICEL CHIRALCEL OD (250 mm*30 mm, 10 μm); mobile phase: [0.1% NH₃H₂O in IPA]; B %: 30%-30%, 4.8 min) to successively afford enantiomer 1 (3a) (230 mg) and enantiomer 2 (3b) (240 mg), both as yellow oils.

### Procedure for the preparation of N-methyl-2-(trifluoromethyl)-4,5,6,7-tetrahydrobenzo thiophen-6-amine hydrochloride (38)

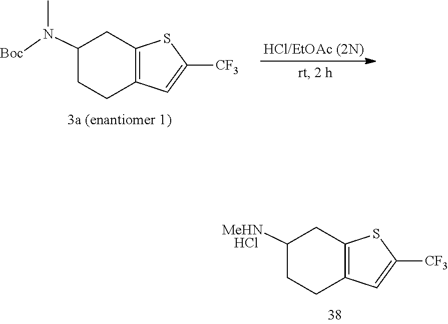 [SMILES: CN(C(=O)OC(C)(C)C)C1CCc2cc(C(F)(F)F)sc2C1 ; Cl ; CNC1CCc2cc(C(F)(F)F)sc2C1]

To a solution of tert-butyl N-methyl-N-[2-(trifluoromethyl)-4,5,6,7-tetrahydrobenzothiophen-6-yl]carbamate (3a, enantiomer 1) (230 mg, 686 μmol, 1 eq) in EtOAc (5 mL) was added HCl/EtOAc (4 M, 5 mL). The mixture was stirred at room temperature for 2 h. The reaction mixture was concentrated to dryness under reduced pressure. The crude product was purified by trituration with EtOAc to give N-methyl-2-(trifluoromethyl)-4,5,6,7-tetrahydrobenzothiophen-6-amine (38) (146.21 mg, 538 μmol, 78.5% yield, HCl salt) as a white solid. ¹H NMR (400 MHz, Methanol-d4) δ (ppm) 7.30 (s, 1H), 3.69-3.56 (m, 1H), 3.40 (dd, J=5.2, 16.4 Hz, 1H), 3.01-2.74 (m, 6H), 2.37-2.26 (m, 1H), 2.03-1.89 (m, 1H). LCMS: m/z [M+H]⁺=236.0.

### Procedure for preparation of N-methyl-2-(trifluoromethyl)-4,5,6,7-tetrahydrobenzo thiophen-6-amine hydrochloride (39)

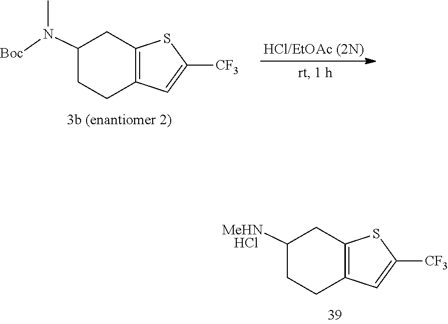 [SMILES: CN(C(=O)OC(C)(C)C)C1CCc2cc(C(F)(F)F)sc2C1 ; Cl ; CNC1CCc2cc(C(F)(F)F)sc2C1]

To a solution of tert-butyl N-methyl-N-[2-(trifluoromethyl)-4,5,6,7-tetrahydrobenzothiophen-6-yl]carbamate (3b, enantiomer 2) (240 mg, 715 μmol, 1 eq) in EtOAc (5 mL) was added HCl/EtOAc (4 M, 5 mL). The mixture was stirred at room temperature for 2 h. The reaction mixture was concentrated to dryness under reduced pressure. The crude product was purified by trituration with EtOAc to give N-methyl-2-(trifluoromethyl)-4,5,6,7-tetrahydrobenzothiophen-6-amine (39) (159.46 mg, 587 μmol, 82.0% yield, HCl salt) as a white solid. ¹H NMR (400 MHz, Methanol-d4) δ (ppm)=7.28 (s, 1H), 3.66-3.55 (m, 1H), 3.38 (dd, J=4.8, 16.0 Hz, 1H), 2.99-2.73 (m, 6H), 2.36-2.24 (m, 1H), 2.02-1.89 (m, 1H). LCMS: m/z [M+H]⁺=236.0.

### Synthetic scheme for the preparation of N, 3-dimethyl-4,5,6,7-tetrahydro-2-benzo thiophen-5-amine hydrochloride enantiomer 1 (40) and enantiomer 2 (41)

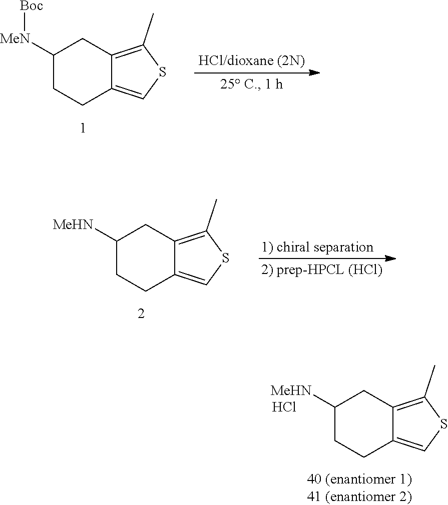 [SMILES: Cc1scc2c1CC(N(C)C(=O)OC(C)(C)C)CC2 ; CNC1CCc2csc(C)c2C1 ; Cl ; CNC1CCc2csc(C)c2C1]

The synthesis of tert-butyl methyl(1-methyl-4,5,6,7-tetrahydrobenzo[c]thiophen-5-yl)carbamate (1) was as described for the synthesis of 32 and 33.

### Procedure for the preparation of N, 3-dimethyl-4,5,6,7-tetrahydro-2-benzothiophen-5-amine hydrochloride enantiomer 1 (40) and enantiomer 2 (41)

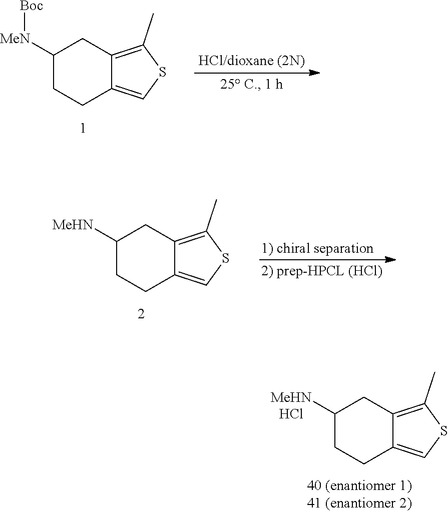 [SMILES: Cc1scc2c1CC(N(C)C(=O)OC(C)(C)C)CC2 ; CNC1CCc2csc(C)c2C1 ; Cl ; CNC1CCc2csc(C)c2C1]

To a solution of tert-butyl N-methyl-N-(3-methyl-4,5,6,7-tetrahydro-2-benzothiophen-5-yl) carbamate (1) (300 mg, 1.07 mmol, 1 eq) in EtOAc (4 mL) was added HCl/dioxane (4 M, 4 mL). The mixture was stirred at 25° C. for 1 hr. The reaction mixture was concentrated under reduced pressure to afford the racemate, which was separated by prep-Chiral-SFC (Column: Phenomenex-Cellulose-2 (250 mm*30 mm, 10 μm); mobile phase: [0.1% NH₃H₂O in EtOH]; B %: 45%-45%, 4.5 min) to give enantiomer 1 (1^{st} peak in SFC) and enantiomer 2 (2^{nd} peak in SFC).

Enantiomer 1 was subjected to prep-HPLC (Column: 3_Phenomenex Luna C18 75*30 mm*3 μm; mobile phase: [water (HCl)-ACN]; B %: 4%-24%, 8 min) and lyophilized to afford N, 3-dimethyl-4,5,6,7-tetrahydro-2-benzothiophen-5-amine hydrochloride salt (40) (97.49 mg, 443.22 μmol, 41.58% yield) as a white solid. ¹H NMR (400 MHz, METHANOL-d₄) δ 6.77 (s, 1H), 3.45 (tdd, J=2.7, 10.4, 13.0 Hz, 1H), 3.17 (dd, J=5.3, 15.4 Hz, 1H), 2.93 (td, J=4.8, 16.6 Hz, 1H), 2.82-2.68 (m, 4H), 2.55 (dd, J=9.9, 15.5 Hz, 1H), 2.34 (s, 3H), 2.27-2.19 (m, 1H), 1.86-1.74 (m, 1H). LCMS: m/z [M+H]⁺=182.0.

Enantiomer 2 was subjected to prep-HPLC (Column: 3_Phenomenex Luna C18 75*30 mm*3 μm; mobile phase: [water (HCl)-ACN]; B %: 4%-24%, 8 min) to afford N, 3-dimethyl-4,5,6,7-tetrahydro-2-benzothiophen-5-amine hydrochloride salt (41) (94.78 mg, 430.90 μmol, 40.42% yield) as a white solid. ¹H NMR (400 MHz, METHANOL-d₄) δ 6.78 (s, 1H), 3.45 (ddt, J=2.8, 5.4, 10.3 Hz, 1H), 3.17 (dd, J=5.4, 15.4 Hz, 1H), 2.93 (td, J=4.7, 16.8 Hz, 1H), 2.85-2.68 (m, 4H), 2.55 (dd, J=9.9, 15.6 Hz, 1H), 2.34 (s, 3H), 2.27-2.18 (m, 1H), 1.80 (dtd, J=5.3, 11.0, 12.7 Hz, 1H). LCMS: m/z [M+H]⁺=182.0.

### Synthetic scheme for the preparation of N, 2,3-trimethyl-4,5,6,7-tetrahydrobenzothiophen-6-amine hydrochloride enantiomer 1 (42) and enantiomer 2 (43)

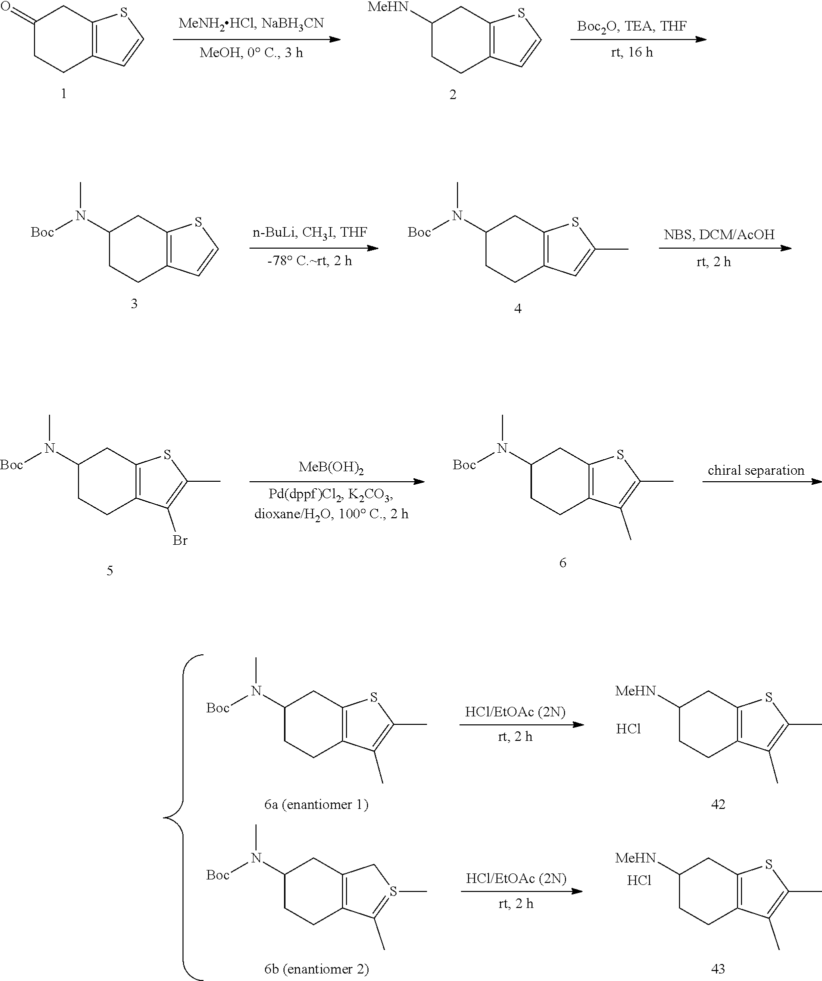

### Procedure for the preparation of N-methyl-4,5,6,7-tetrahydrobenzothiophen-6-amine (2)

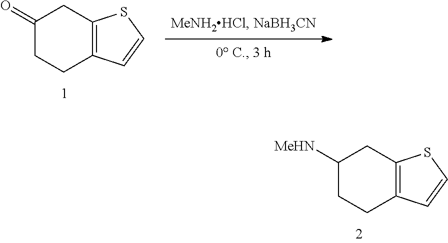 [SMILES: O=C1CCc2ccsc2C1 ; CNC1CCc2ccsc2C1]

A mixture of 5,7-dihydro-4H-benzothiophen-6-one (1) (9.0 g, 59.13 mmol, 1 eq) and methanamine hydrochloride salt (11.98 g, 177.38 mmol, 3 eq, HCl) in MeOH (300 mL) was stirred at 0° C. for 0.5 hr before being treated with NaBH₃CN (22.3 g, 354.77 mmol, 6 eq) batch-wise at 0° C., and the resulting mixture was stirred at 0° C. for 2.5 hr. The reaction mixture was concentrated under reduced pressure, diluted with H₂O (200 mL), and extracted with EtOAc (200 mL*2). The combined organic layers were washed with washed with brine, dried over anhydrous sodium sulfate, filtered, and concentrated under reduced pressure to give N-methyl-4,5,6,7-tetrahydrobenzothiophen-6-amine (2) (11 g, crude) as a brown oil.

### Procedure for the preparation of tert-butyl N-methyl-N-(4,5,6,7-tetrahydrobenzothiophen-6-yl) carbamate (3)

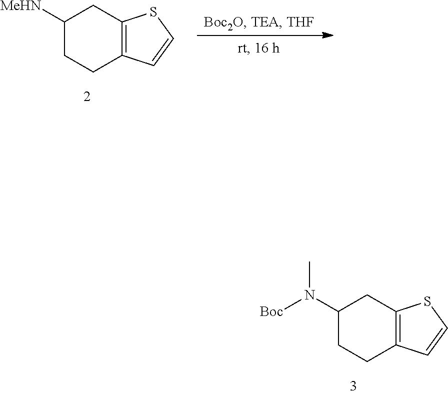 [SMILES: CNC1CCc2ccsc2C1 ; CN(C(=O)OC(C)(C)C)C1CCc2ccsc2C1]

To a solution of N-methyl-4,5,6,7-tetrahydrobenzothiophen-6-amine (2) (11 g crude) and TEA (13.3 g, 131.52 mmol, 18.3 mL, 2 eq) in THF (200 mL) was added Boc₂O (28.70 g, 131.52 mmol, 30.2 mL, 2 eq) drop-wise at 25° C., and the resulting mixture was stirred for 16 hr at that temperature. The mixture was poured into H₂O (200 mL) and extracted with EtOAc (300 mL*3). The combined organic layer was dried over Na₂SO₄, filtered, and concentrated in vacuo. The residue was purified by flash silica gel chromatography (Silica Flash Column, eluent 0~20% Ethyl acetate/Petroleum ether) to afford tert-butyl N-methyl-N-(4,5,6,7-tetrahydrobenzothiophen-6-yl)carbamate (3) (11.2 g, 41.89 mmol, 70.8% yield over two steps) as a white solid. ¹H NMR (400 MHz, DMSO-d₆) δ ppm 7.26 (d, J=5.01 Hz, 1H), 6.78 (d, J=5.01 Hz, 1H), 4.07-4.36 (m, 1H), 2.57-2.93 (m, 7H), 1.72-1.94 (m, 2H), 1.41 (s, 9H).

### Procedure for the preparation of tert-butyl N-methyl-N-(2-methyl-4,5,6,7-tetrahydrobenzo thiophen-6-yl) carbamate (4)

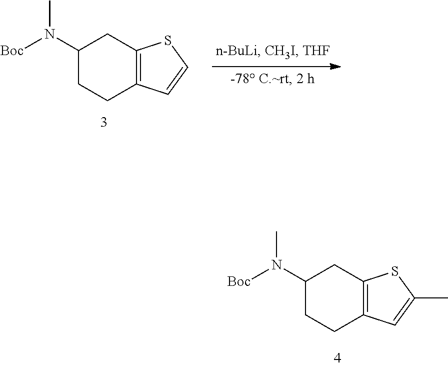 [SMILES: CN(C(=O)OC(C)(C)C)C1CCc2ccsc2C1 ; Cc1cc2c(s1)CC(N(C)C(=O)OC(C)(C)C)CC2]

To a solution of tert-butyl N-methyl-N-(4,5,6,7-tetrahydrobenzothiophen-6-yl)carbamate (3) (3.0 g, 11.22 mmol, 1 eq) in THF (30 mL) was added dropwise n-BuLi (2.5 M in hexanes, 5.39 mL, 13.48 mmol, 1.2 eq) at −78° C. under N₂ atmosphere. The resulting mixture was stirred at −78° C. for 0.5 hr before being treated with CH₃I (2.39 g, 16.83 mmol, 1.05 mL, 1.5 eq) drop-wise, and the reaction mixture was warmed to ambient temperature and stirred for 1.5 hr. The reaction mixture quenched with sat.NH₄Cl solution (50 mL) and extracted with EtOAc (50 mL*3). The combined organic was dried over Na₂SO₄, filtered, and concentrated in vacuo to afford tert-butyl N-methyl-N-(2-methyl-4,5,6,7-tetrahydrobenzothiophen-6-yl)carbamate (4) (3.2 g, crude) as yellow oil. ¹H NMR (400 MHz, CHLOROFORM-d) δ ppm 6.40 (s, 1H), 2.57-2.92 (m, 8H), 2.41 (s, 3H), 1.82-1.93 (m, 2H), 1.47 (s, 10H).

### Procedure for the preparation of tert-butyl N-(3-bromo-2-methyl-4,5,6,7-tetrahydrobenzo thiophen-6-yl)-N-methyl-carbamate (5)

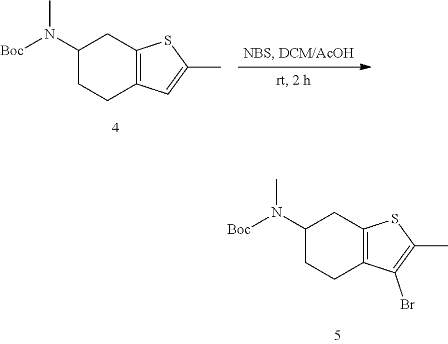 [SMILES: Cc1cc2c(s1)CC(N(C)C(=O)OC(C)(C)C)CC2 ; Cc1sc2c(c1Br)CCC(N(C)C(=O)OC(C)(C)C)C2]

To a solution of tert-butyl N-methyl-N-(2-methyl-4,5,6,7-tetrahydrobenzothiophen-6-yl)carbamate (4) (1.0 g, 3.55 mmol, 1 eq) in DCM (5 mL) and AcOH (5 mL) was added NBS (696 mg, 3.91 mmol, 1.1 eq), and the resulting mixture was stirred at room temperature for 2 h. The reaction mixture was poured into H₂O (50 mL) and extracted with EtOAc (50 mL*3). The combined organic layers were washed with aq. sat. NaCl (20 mL), dried over anhydrous Na₂SO₄, filtered and concentrated under reduced pressure. The residue was purified by flash silica gel chromatography (ISCO®; 12 g SepaFlash® Silica Flash Column, eluent 0~10% Ethyl acetate/Petroleum ether) to give tert-butyl N-(3-bromo-2-methyl-4,5,6,7-tetrahydrobenzothiophen-6-yl)-N-methylcarbamate (5) (800 mg, 1.87 mmol, 52.5% yield) as a yellow solid.

### Procedure for the preparation of tert-butyl N-(2,3-dimethyl-4,5,6,7-tetrahydrobenzothio phen-6-yl)-N-methyl-carbamate enantiomer 1 (6a) and enantiomer 2 (6b)

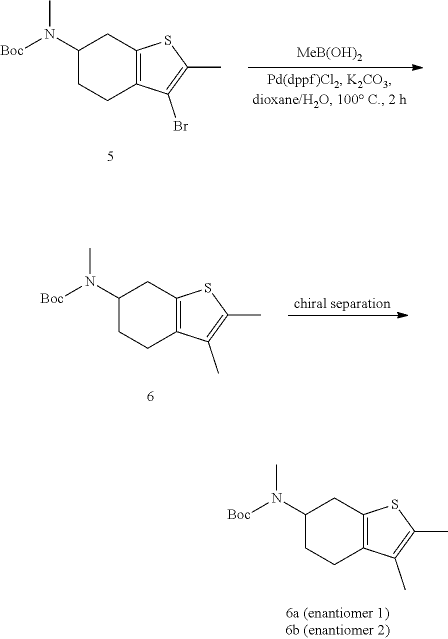

To a solution of tert-butyl N-(3-bromo-2-methyl-4,5,6,7-tetrahydrobenzothiophen-6-yl)-N-methyl-carbamate (5) (750 mg, 2.08 mmol, 1 eq) in dioxane (5 mL) and H₂O (1 mL) were added Pd(dppf)Cl₂ (152 mg, 208 μmol, 0.1 eq), MeB(OH)₂ (374 mg, 6.24 mmol, 3 eq) and K₂CO₃ (575 mg, 4.16 mmol, 2 eq), and the resulting mixture was stirred at 100° C. for 2 h under N₂ atmosphere. The reaction mixture cooled to rt, diluted with H₂O (10 mL) and extracted with EtOAc (10 mL*3). The combined organic layers were washed with sat. NaCl (10 mL), dried over anhydrous Na₂SO₄, filtered and concentrated under reduced pressure. The residue was purified by prep-HPLC (column: Waters Xbridge 150*25 mm*5 μm; mobile phase: [water (NH₄HCO₃)-ACN]; B %: 60%-90%, 8 min) to give the racemic tert-butyl N-(2,3-dimethyl-4,5,6,7-tetrahydrobenzothiophen-6-yl)-N-methyl-carbamate (6), which was further separated by prep-Chiral-SFC (column: DAICEL CHIRALPAK AD (250 mm*30 mm, 10 μm); mobile phase: [0.1% NH₃H₂O in MEOH]; B %: 30%-30%, 2.6 min) to successively afford enantiomer 1 (6a) (170 mg, 572 μmol, 27.5% yield) and enantiomer 2 (6b) (150 mg, 508 μmol, 24.4% yield), both as white solids. LCMS: m/z [M+H-Boc]⁺=195.9.

### Procedure for the preparation of N, 2,3-trimethyl-4,5,6,7-tetrahydrobenzothiophen-6-amine hydrochloride enantiomer 1 (42)

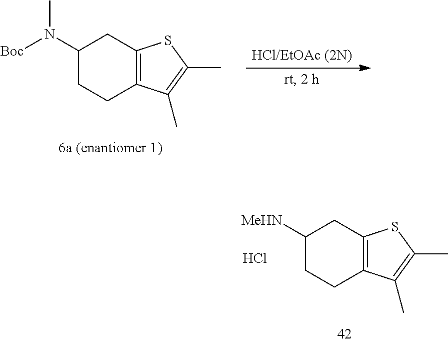 [SMILES: Cc1sc2c(c1C)CCC(N(C)C(=O)OC(C)(C)C)C2 ; Cl ; CNC1CCc2c(sc(C)c2C)C1]

To a solution of tert-butyl N-(2,3-dimethyl-4,5,6,7-tetrahydrobenzothiophen-6-yl)-N-methyl-carbamate (6a) (170 mg, 575 μmol, 1 eq) in EtOAc (5 mL) was added HCl/EtOAc (4 M, 5 mL). The mixture was stirred at room temperature for 2 h. The reaction mixture was concentrated to dryness under reduced pressure. The crude product was purified by trituration with EtOAc to give N, 2,3-trimethyl-4,5,6,7-tetrahydrobenzothiophen-6-amine hydrochloride salt enantiomer 1 (42) (112 mg, 483 μmol, 84.0% yield) as a white solid. ¹H NMR (400 MHz, Methanol-d4) δ (ppm)=3.58-3.45 (m, 1H), 3.19 (dd, J=4.8, 15.6 Hz, 1H), 2.86-2.75 (m, 4H), 2.71-2.52 (m, 2H), 2.33-2.19 (m, 4H), 2.01-1.87 (m, 4H). LCMS: m/z [M+H]⁺=196.1.

### Procedure for the preparation of N, 2,3-trimethyl-4,5,6,7-tetrahydrobenzothiophen-6-amine hydrochloride enantiomer 2 (43)

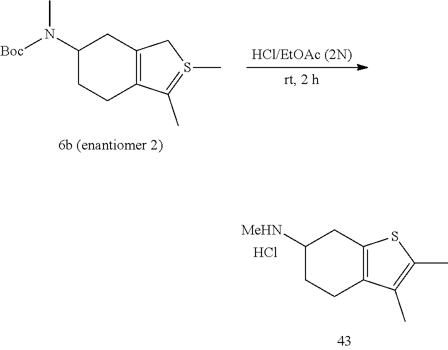 [SMILES: CC1=S(C)CC2=C1CCC(N(C)C(=O)OC(C)(C)C)C2 ; Cl ; CNC1CCc2c(sc(C)c2C)C1]

To a solution of tert-butyl N-(2,3-dimethyl-4,5,6,7-tetrahydrobenzothiophen-6-yl)-N-methyl-carbamate (6b) (150 mg, 508 μmol, 1 eq) in EtOAc (5 mL) was added HCl/EtOAc (4 M, 5 mL). The mixture was stirred at 25° C. for 2 h. The reaction mixture was concentrated to dryness under reduced pressure. The crude product was purified by trituration with EtOAc to give N, 2,3-trimethyl-4,5,6,7-tetrahydrobenzothiophen-6-amine hydrochloride salt enantiomer 2 (43) (114.20 mg, 493 μmol, 97.0% yield) as a white solid. ¹H NMR (400 MHz, Methanol-d4) δ (ppm)=3.57-3.46 (m, 1H), 3.19 (dd, J=4.8, 16.0 Hz, 1H), 2.85-2.74 (m, 4H), 2.71-2.52 (m, 2H), 2.28-2.20 (m, 4H), 2.01-1.85 (m, 4H). LCMS: m/z [M+H]⁺=196.1.

### Synthetic scheme for the preparation of N, 3-dimethyl-4,5,6,7-tetrahydrobenzothiophen-6-amine hydrochloride enantiomer 1 (44) and enantiomer 2 (45)

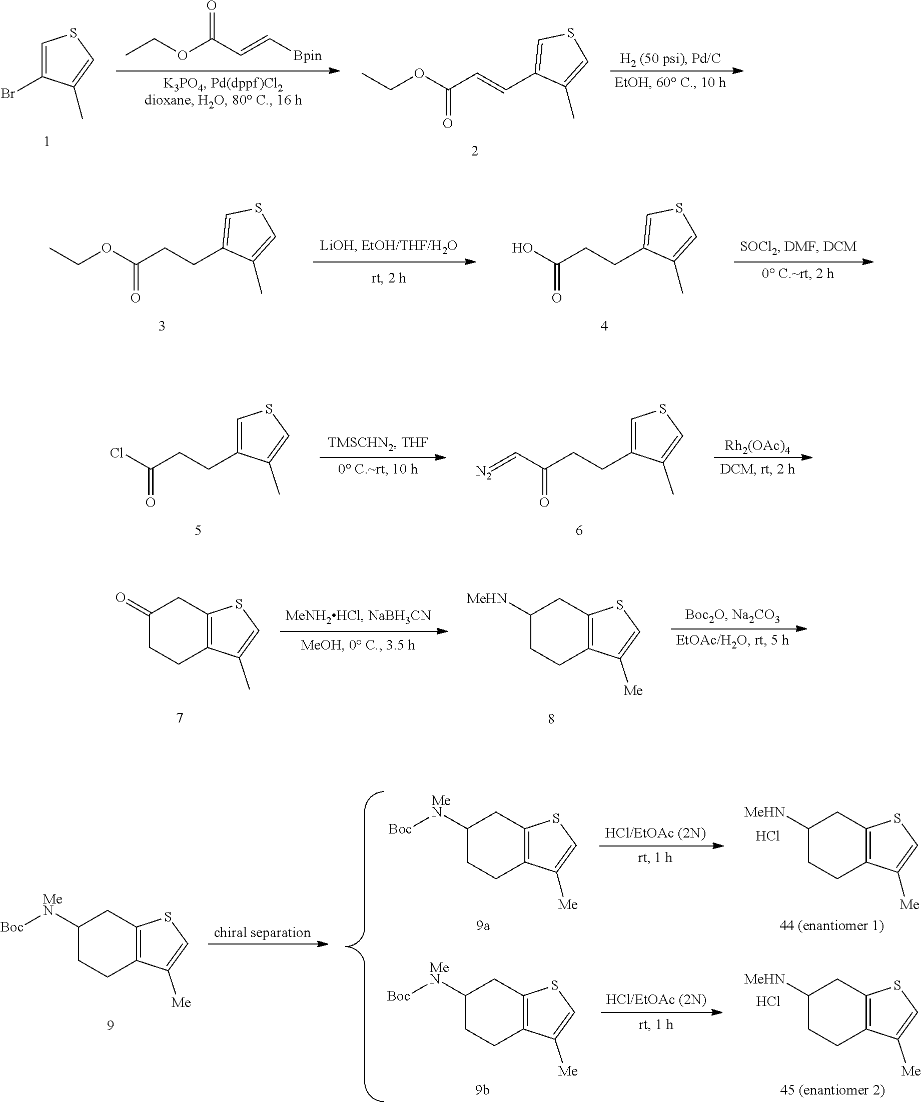

### Procedure for preparation of ethyl (E)-3-(4-methyl-3-thienyl) prop-2-enoate (2)

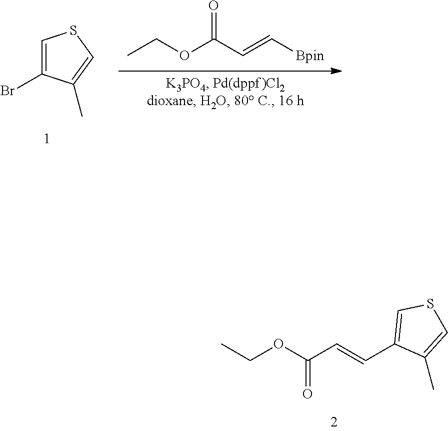

A mixture of 3-bromo-4-methyl-thiophene (1) (11 g, 62.13 mmol, 1 eq), ethyl (E)-3-(4,4,5,5-tetramethyl-1,3,2-dioxaborolan-2-yl)prop-2-enoate (22.47 g, 99.40 mmol, 1.6 eq), K₃PO₄ (32.97 g, 155.31 mmol, 2.5 eq) and Pd(dppf)Cl₂ (2.27 g, 3.11 mmol, 0.05 eq) in dioxane (200 mL) and H₂O (20 mL) was degassed and purged with N₂ for 3 times, and then the mixture was stirred at 80° C. for 16 h under N₂ atmosphere. The reaction mixture was cooled to rt, filtered, and the filtrate was diluted with H₂O (100 mL) and extracted with EtOAc (100 mL*2). The combined organic layers were washed with brine, dried over anhydrous sodium sulfate, filtered, and concentrated under reduced pressure. The residue was purified by flash silica gel chromatography (ISCO®; 80 g SepaFlash® Silica Flash Column, eluent 0~5% Ethyl acetate/Petroleum ether) to give ethyl (E)-3-(4-methyl-3-thienyl) prop-2-enoate (2) (11.3 g, 57.58 mmol, 92.68% yield) as a yellow oil. ¹H NMR (400 MHz, CHLOROFORM-d) δ 7.67 (d, J=16.0 Hz, 1H), 7.55 (d, J=3.2 Hz, 1H), 6.97-6.96 (m, 1H), 6.30 (d, J=16.0 Hz, 1H), 4.27 (q, J=7.2 Hz, 2H), 2.34 (s, 3H), 1.34 (t, J=7.2 Hz, 3H).

### Procedure for the Preparation of Ethyl 3-(4-Methyl-3-Thienyl) Propanoate (3)

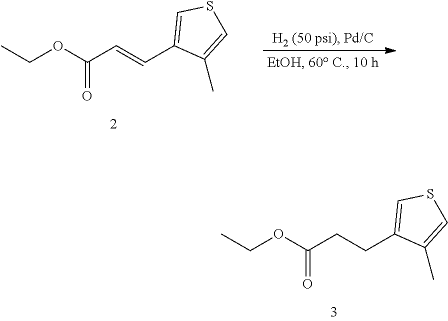

To a solution of ethyl (E)-3-(4-methyl-3-thienyl) prop-2-enoate (2) (11.3 g, 57.58 mmol, 1 eq) in EtOH (100 mL) was added 10% Pd/C (5 g) under N₂. The suspension was degassed under vacuum and purged with H₂ several times. The mixture was stirred under H₂ (50 psi) at 60° C. for 10 hours. The reaction mixture was cooled to rt, filtered through a pad of Celite, and the filtrate was concentrated to give ethyl 3-(4-methyl-3-thienyl) propanoate (3) (11.3 g, quant.) as a yellow oil. LCMS: m/z [M+H]⁺=199.2.

### Procedure for preparation of 3-(4-methyl-3-thienyl)propanoic acid (4)

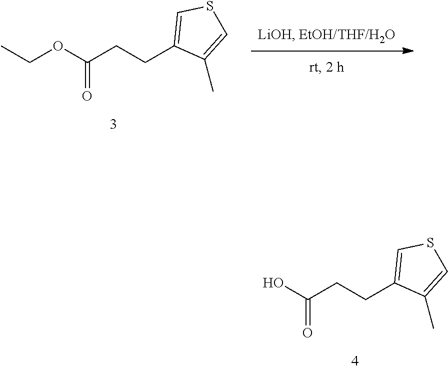

To a solution of ethyl 3-(4-methyl-3-thienyl) propanoate (3) (11.3 g, 56.99 mmol, 1 eq) in THF (80 mL), H₂O (80 mL) and EtOH (10 mL) was added LiOH·H₂O (4.78 g, 113.98 mmol, 2 eq). The mixture was stirred at 20° C. for 2 hr. The reaction mixture was concentrated under reduced pressure to remove organic solvents, treated with 2 N HCl until pH 6, and extracted with EtOAc (50 mL*3). The combined organic layers were washed with brine (50 mL), dried over anhydrous Na₂SO₄, filtered, and concentrated under reduced pressure to give 3-(4-methyl-3-thienyl) propanoic acid (4) (8.4 g, 86.6%) as a yellow solid.

### Procedure for the preparation of 1-diazo-4-(4-methyl-3-thienyl) butan-2-one (6) and 1-diazo-4-(4-methyl-3-thienyl)-1-trimethylsilyl-butan-2-one (6a)

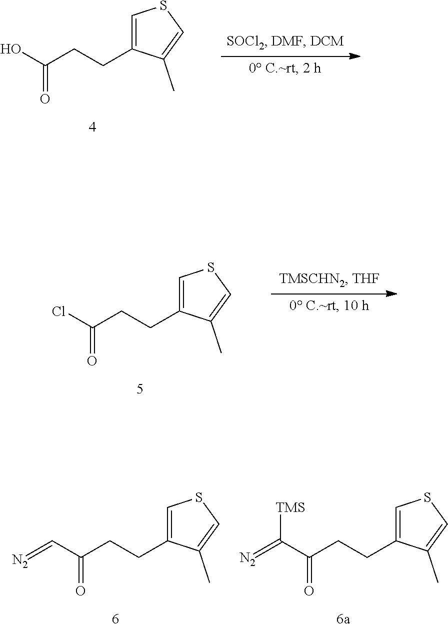

To a solution of 3-(4-methyl-3-thienyl) propanoic acid (4) (6.4 g, 37.60 mmol, 1 eq) and DMF (50 mg, 752 μmol, 58 μL, 0.02 eq) in DCM (60 mL) was added and SOCl₂ (8.95 g, 75.19 mmol, 5.45 mL, 2 eq) drop-wise at 0° C., and the resulting mixture was stirred at 20° C. for 2 hr. The reaction mixture was concentrated under reduced pressure to give 3-(4-methyl-3-thienyl)propanoyl chloride (5) as a brown oil.

To a solution of 3-(4-methyl-3-thienyl) propanoyl chloride (5) in THF (60 mL) was added TMSCHN₂ (2 M in n-hexane, 47.70 mL, 95.4 mmol) drop-wise at 0° C., and the resulting mixture was stirred at 20° C. for 10 hr. The reaction mixture was quenched with sat.NaHCO₃ (60 mL), and extracted with EtOAc (40 mL*2). The combined organic layers were washed with brine (60 mL), dried over anhydrous Na₂SO₄, filtered and concentrated under reduced pressure. The residue was subjected to flash silica gel chromatography (ISCO®; 40 g SepaFlash® Silica Flash Column, eluent 0~10% Ethyl acetate/Petroleum ether) to give 1-diazo-4-(4-methyl-3-thienyl)butan-2-one (6) (2.5 g, 7.98 mmol, 25.09% yield) as a brown oil and 1-diazo-4-(4-methyl-3-thienyl)-1-trimethylsilyl-butan-2-one (6a) (5.52 g, 8.67 mmol, 27.26% yield) as a brown oil.

1-diazo-4-(4-methyl-3-thienyl)butan-2-one (6): ¹H NMR (400 MHz, CHLOROFORM-d) δ 6.98-6.86 (m, 2H), 5.25 (br s, 1H), 2.87 (t, J=7.7 Hz, 2H), 2.73-2.60 (m, 2H), 2.21 (s, 3H).

1-diazo-4-(4-methyl-3-thienyl)-1-trimethylsilyl-butan-2-one (6a): ¹H NMR (400 MHz, CHLOROFORM-d) δ 6.91 (s, 2H), 2.90-2.86 (m, 2H), 2.82-2.77 (m, 2H), 2.22 (s, 3H), 0.29 (s, 9H).

### Procedure for the preparation of 1-diazo-4-(4-methyl-3-thienyl)butan-2-one (6)

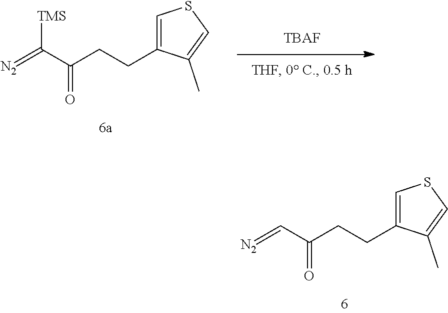

To a solution of 1-diazo-4-(4-methyl-3-thienyl)-1-trimethylsilyl-butan-2-one (6a) (5.5 g, 20.64 mmol, 1 eq) in THF (40 mL) was added TBAF (1 M in THF, 2.06 mL, 2.06 mmol, 0.1 eq) drop-wise at 0° C. The mixture was stirred at 0° C. for 0.5 hr before being concentrated under reduced pressure. The residue was purified by flash silica gel chromatography (ISCO®; 40 g SepaFlash® Silica Flash Column, eluent 0~10% Ethyl acetate/Petroleum ether) to give 1-diazo-4-(4-methyl-3-thienyl) butan-2-one (6) (1.7 g, 8.14 mmol, 39.43% yield) as a yellow oil.

### Procedure for the preparation of 3-methyl-5,7-dihydro-4H-benzothiophen-6-one (7)

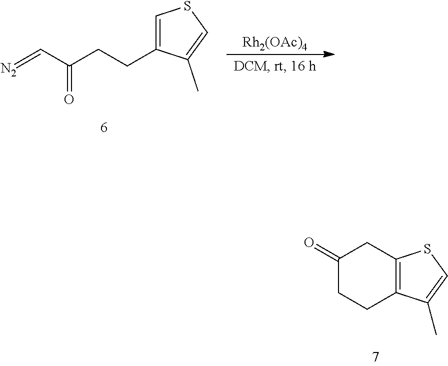

To a solution of 1-diazo-4-(4-methyl-3-thienyl) butan-2-one (6) (4.0 g, 20.59 mmol, 1 eq) in DCM (300 mL) was added Rh₂(OAc)₄ (50 mg, 113 μmol, 0.0055 eq). The resulting mixture was stirred at 20° C. for 10 h under N₂ before being concentrated under reduced pressure. The residue was purified by flash silica gel chromatography (ISCO®; 40 g SepaFlash® Silica Flash Column, eluent 0~10% Ethyl acetate/Petroleum ether) to give 3-methyl-5,7-dihydro-4H-benzothiophen-6-one (7) (1.5 g, 8.48 mmol, 41.19% yield) as a yellow oil. ¹H NMR (400 MHz, CHLOROFORM-d) δ 6.81 (s, 1H), 3.62 (s, 2H), 2.94-2.85 (m, 2H), 2.73-2.65 (m, 2H), 2.17 (s, 3H). LCMS: m/z [M+H]⁺=167.5.

### Procedure for the preparation of N, 3-dimethyl-4,5,6,7-tetrahydrobenzothiophen-6-amine (8)

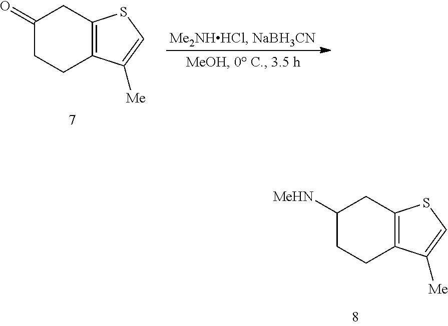

To a mixture of 3-methyl-5,7-dihydro-4H-benzothiophen-6-one (7) (1.0 g, 6.02 mmol, 1 eq) and methanamine hydrochloride (1.22 g, 18.05 mmol, 3 eq, HCl) in MeOH (30 mL) was added NaBH₃CN (2.27 g, 36.09 mmol, 6 eq) batch-wise at 0° C., the mixture was stirred at 0° C. for 3.5 hr. The reaction mixture was quenched with NaHCO₃ (50 mL) and extracted with EtOAc (20 mL*2). The combined organic layers were washed with brine (15 mL), dried over anhydrous sodium sulfate, filtered, and concentrated under reduced pressure to afford N, 3-dimethyl-4,5,6,7-tetrahydrobenzothiophen-6-amine (8) (1 g, crude) as a brown oil. LCMS: m/z [M-NHMe]⁺=151.6.

### Procedure for the preparation of tert-butyl N-methyl-N-(3-methyl-4,5,6,7-tetrahydrobenzo thiophen-6-yl)carbamate (9)

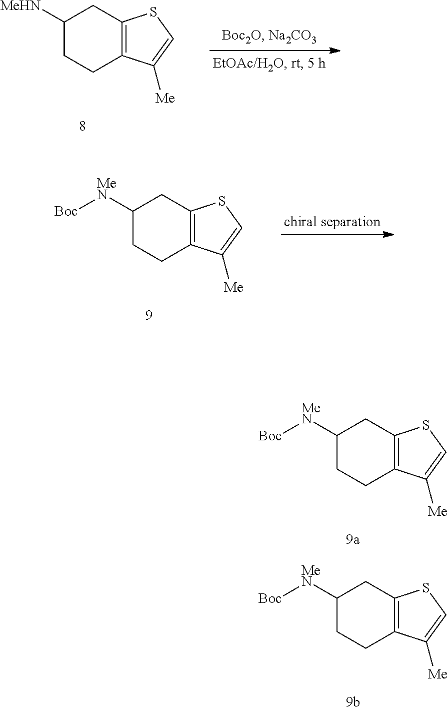 [SMILES: CNC1CCc2c(C)csc2C1 ; Cc1csc2c1CCC(N(C)C(=O)OC(C)(C)C)C2 ; Cc1csc2c1CCC(N(C)C(=O)OC(C)(C)C)C2 ; Cc1csc2c1CCC(N(C)C(=O)OC(C)(C)C)C2]

To a mixture of N, 3-dimethyl-4,5,6,7-tetrahydrobenzothiophen-6-amine (8) (1 g, crude) and Na₂CO₃ (1.2 g, 11.03 mmol, 2 eq) in EtOAc (10 mL) and H₂O (10 mL) was added Boc₂O (2.4 g, 11.03 mmol, 2.53 mL, 2 eq) drop-wise, and the resulting mixture was stirred at 20° C. for 5 hr. The reaction mixture was diluted with H₂O (20 mL) and extracted with EtOAc (20 mL*2). The combined organic layers were washed with brine, dried over anhydrous sodium sulfate, filtered, and concentrated under reduced pressure. The residue was purified by flash silica gel chromatography (ISCO®; 12 g SepaFlash® Silica Flash Column, eluent 0~20% Ethyl acetate/Petroleum ether) to give tert-butyl N-methyl-N-(3-methyl-4,5,6,7-tetrahydrobenzothiophen-6-yl)carbamate (9) (1.1 g, 3.91 mmol, 70.87% yield) as a colorless oil. LCMS: m/z [M+H-tBu]⁺=226.1.

The racemate was separated by prep-Chiral-SFC (Column: DAICEL CHIRALPAK AD (250 mm*30 mm, 10 μm); mobile phase: [0.1% NH₃H₂O in MEOH]; B %: 30%-30%, 4.2 min) to give Enantiomer1 (9a, 1^{st} peak in SFC) and Enantiomer 2 (9b, 2^{nd} peak in SFC).

### Procedure for the preparation of N, 3-dimethyl-4,5,6,7-tetrahydrobenzothiophen-6-amine hydrochloride (44)

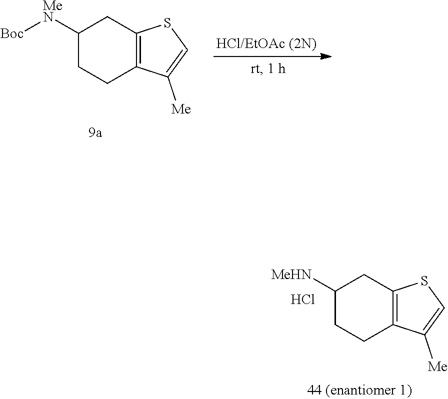 [SMILES: Cc1csc2c1CCC(N(C)C(=O)OC(C)(C)C)C2 ; Cl ; CNC1CCc2c(C)csc2C1]

A mixture of tert-butyl N-methyl-N-(3-methyl-4,5,6,7-tetrahydrobenzothiophen-6-yl) carbamate enantiomer 1 (9a) (200 mg, 710 μmol, 1 eq) in 2 N HCl/EtOAc (8 mL) was stirred at 25° C. for 1 hr. The reaction mixture was concentrated to dryness under reduced pressure, and the crude product was purified by trituration with EtOAc to give N, 3-dimethyl-4,5,6,7-tetrahydrobenzothiophen-6-amine hydrochloride salt enantiomer 1 (44) (154.04 mg, 700 μmol, 98.54% yield) as a white solid. ¹H NMR (400 MHz, METHANOL-d₄) δ 6.84 (s, 1H), 3.58-3.52 (m, 1H), 3.30-3.24 (m, 1H), 2.89 (dd, J=8.6, 16.0 Hz, 1H), 2.79 (s, 3H), 2.76-2.56 (m, 2H), 2.35-2.25 (m, 1H), 2.11 (s, 3H), 2.02-1.90 (m, 1H). LCMS: m/z [M+H]⁺=182.1.

### Procedure for preparation of N, 3-dimethyl-4,5,6,7-tetrahydrobenzothiophen-6-amine hydrochloride (45)

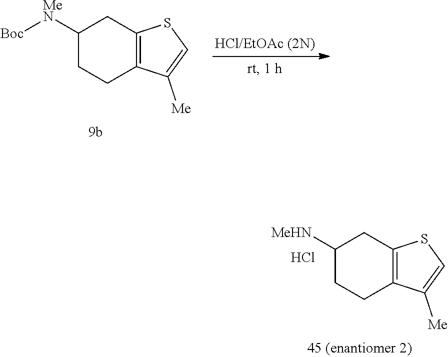 [SMILES: Cc1csc2c1CCC(N(C)C(=O)OC(C)(C)C)C2 ; Cl ; CNC1CCc2c(C)csc2C1]

A mixture of tert-butyl N-methyl-N-(3-methyl-4,5,6,7-tetrahydrobenzothiophen-6-yl) carbamate enantiomer 2 (9b) (250 mg, 888.37 μmol, 1 eq) in 2 N HCl/EtOAc (8 mL) was stirred at 25° C. for 1 hr. The reaction mixture was concentrated to dryness under reduced pressure, and the crude product was purified by trituration with EtOAc to give N, 3-dimethyl-4,5,6,7-tetrahydrobenzothiophen-6-amine hydrochloride salt enantiomer 2 (45) (155.75 mg, 715.24 μmol, 80.51% yield) as a white solid. ¹H NMR (400 MHz, METHANOL-d₄) δ 6.84 (s, 1H), 3.58-3.51 (m, 1H), 3.30-3.24 (m, 1H), 2.98-2.84 (m, 1H), 2.79 (s, 3H), 2.76-2.57 (m, 2H), 2.38-2.25 (m, 1H), 2.11 (s, 3H), 2.04-1.91 (m, 1H). LCMS: m/z [M+H]⁺=182.1.

### Synthetic scheme for the preparation of N-methyl-6,7-dihydro-5H-thieno[3,2-b]pyran-6-amine hydrochloride enantiomer 1 (46) and enantiomer 2 (47)

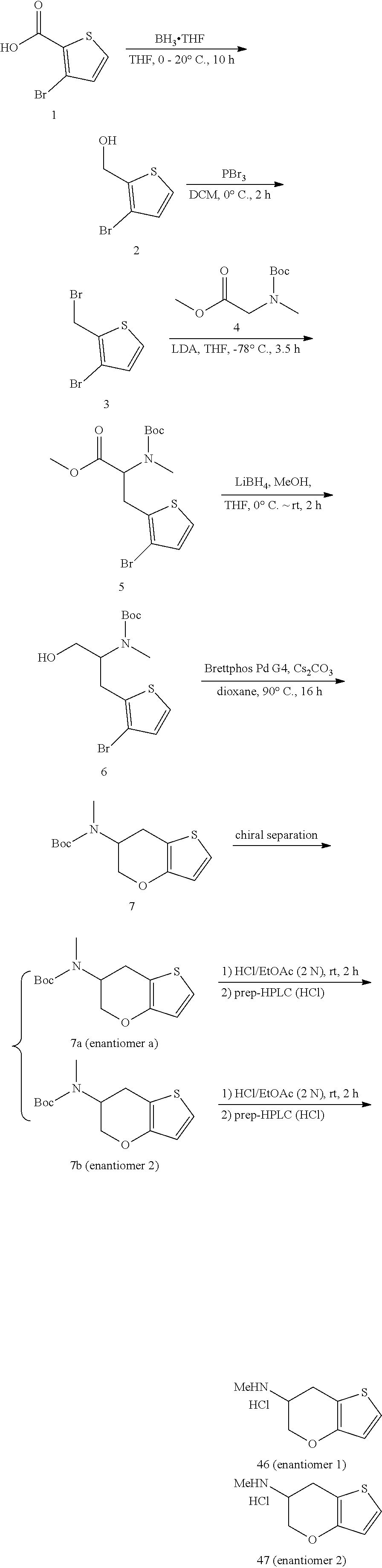

### Procedure for the preparation of (3-bromo-2-thienyl)methanol (2)

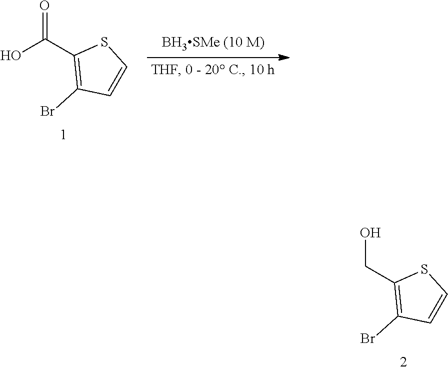 [SMILES: O=C(O)c1sccc1Br ; OCc1sccc1Br]

To a solution of 3-bromothiophene-2-carboxylic acid (17.0 g, 82.11 mmol, 1 eq) in anhydrous THF (200 mL) was added BH₃·SMe₂ (10.0 M, 16.42 mL, 2 eq) dropwise at 0° C. under N₂ atmosphere, and the resulting mixture was stirred at 20° C. for 10 hrs. The reaction mixture was cooled to 0° C., quenched by slowly adding MeOH (20 mL), and then concentrated under reduced pressure to give (3-bromo-2-thienyl)methanol (15.6 g, crude) as a yellow oil.

### Procedure for the preparation of 3-bromo-2-(bromomethyl)thiophene (3)

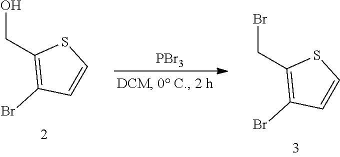 [SMILES: OCc1sccc1Br ; "PBr3" ; BrCc1sccc1Br]

To a solution of (3-bromo-2-thienyl)methanol (2) (15.0 g, 77.70 mmol, 1 eq) in DCM (150 mL) was added PBr₃ (25.24 g, 93.23 mmol, 8.8 mL, 1.2 eq) dropwise at 0° C., and the resulting mixture was stirred at 0° C. for 2 hr. The reaction mixture was quenched by adding into sat. NaHCO₃ (200 mL), and the quenched reaction mixture was extracted with EtOAc (200 mL*2). The combined organic layers were washed with brine (20 mL), dried over anhydrous Na₂SO₄, filtered and concentrated under reduced pressure. The residue was purified by flash silica gel chromatography (eluting with 1:5 Ethyl acetate/Petroleum ether) to afford 3-bromo-2-(bromomethyl)thiophene (3) (12 g, 46.88 mmol, 60.34% yield) as a yellowish oil. ¹H NMR (400 MHz, CDCl₃) δ=7.33 (d, J=5.4 Hz, 1H), 6.96 (d, J=5.3 Hz, 1H), 4.70 (s, 2H).

### Procedure for the preparation of methyl 3-(3-bromo-2-thienyl)-2-[tert-butoxycarbonyl (methyl) amino]propanoate (5)

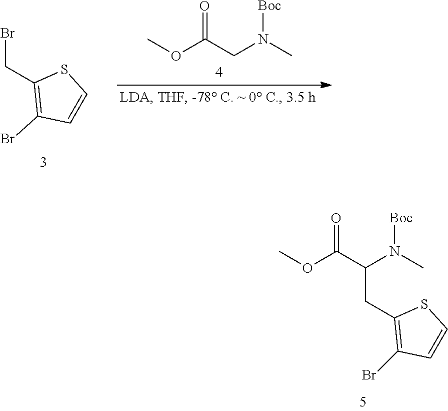

To a solution of LDA (2 M in THF, 28.13 mL, 1.2 eq) in THF (10 mL) was added a solution of methyl 2-[tert-butoxycarbonyl(methyl)amino]acetate (11.43 g, 56.26 mmol, 1.2 eq) in THF (20 mL) dropwise at −78° C. under N₂ atmosphere, and the resulting mixture was stirred at that temperature for 0.5 h. A solution of 3-bromo-2 (bromomethyl)thiophene (3) (12 g, 46.88 mmol, 1 eq) in THF (20 mL) was added dropwise, and the resulting mixture was stirred at −78° C. for 2 hr before being warmed to 0° C. and stirred for another 1 h. The reaction mixture was quenched with saturated NH₄Cl solution (100 mL) and extracted with EtOAc (50 mL*3). The combined organic phase was washed with brine (10 mL*3), dried over Na₂SO₄, filtered and concentrated under reduced pressure. The residue was purified by silica gel chromatography (eluting with 1:5 Ethyl acetate/Petroleum ether) to afford 3-(3-bromo-2-thienyl)-2-[tert-butoxycarbonyl(methyl)amino]propanoate (5) (8.9 g, 23.53 mmol, 50.18% yield) as a yellowish oil.

### Procedure for preparation of tert-butyl N-[1-[(3-bromo-2-thienyl)methyl]-2-hydroxy-ethyl]-N-methyl-carbamate (6)

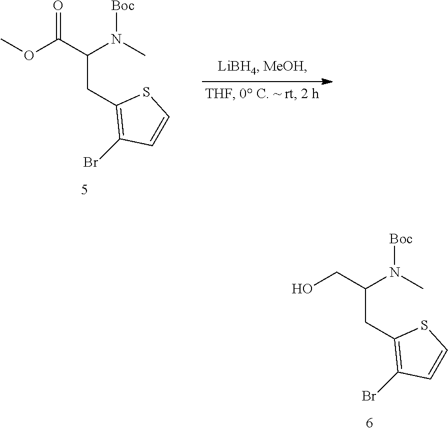 [SMILES: COC(=O)C(Cc1sccc1Br)N(C)C(=O)OC(C)(C)C ; CN(C(=O)OC(C)(C)C)C(CO)Cc1sccc1Br]

To a suspension of LiBH₄ (1.3 g, 59.69 mmol, 2.54 eq) in THF (20 mL) was added a solution of methyl 3-(3-bromo-2-thienyl)-2-[tert-butoxycarbonyl(methyl)amino]propanoate (5) (8.9 g, 23.53 mmol, 1 eq) in THF (80 mL) dropwise at 0° C. Then MeOH (1 mL) was added, and the resulting mixture was stirred at 20° C. for 2 hrs. The reaction mixture was quenched with sat. NH₄Cl solution (100 mL) and extracted with EtOAc (50 mL*3). The combined organic phase was washed with brine (10 mL*3), dried over Na₂SO₄, filtered, and concentrated under reduced pressure. The residue was purified by silica gel chromatography (eluting with 1:1 Ethyl acetate/Petroleum ether) to afford tert-butyl N-[1-[(3-bromo-2-thienyl) methyl]-2-hydroxy-ethyl]-N-methyl-carbamate (6) (5.9 g, 16.84 mmol, 71.59% yield) as a yellowish oil.

### Procedure for the preparation of tert-butyl N-[1-[(3-bromo-2-thienyl)methyl]-2-hydroxy-ethyl]-N-methyl-carbamate (7)

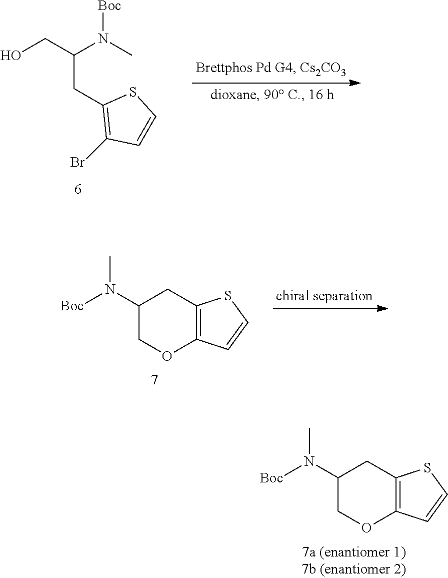 [SMILES: CN(C(=O)OC(C)(C)C)C(CO)Cc1sccc1Br ; CN(C(=O)OC(C)(C)C)C1COc2ccsc2C1 ; CN(C(=O)OC(C)(C)C)C1COc2ccsc2C1]

To a solution of tert-butyl N-[1-[(3-bromo-2-thienyl) methyl]-2-hydroxy-ethyl]-N-methyl-carbamate (6) (5.6 g, 15.99 mmol, 1 eq) in dioxane (1 mL) were added Brettphos Pd G4 (736 mg, 799 μmol, 0.05 eq) and Cs₂CO₃ (10.42 g, 31.98 mmol, 2 eq), and the resulting mixture was stirred at 90° C. for 16 hr under N₂. The reaction mixture was cooled to rt and concentrated in vacuo. The residue was purified by silica gel chromatography (100-200 mesh silica gel, Petroleum ether/Ethyl acetate=5/1) to afford the racemic tert-butyl N-(6, 7-dihydro-5H-thieno[3, 2-b]pyran-6-yl)-N-methyl-carbamate (7) (165 mg, 588.06 μmol, 3.81% yield, 96% purity), which was further separated by prep-Chiral-SFC (Column: Chiralpak AD-3 50×4.6 mm I. D., 3 um Mobile phase: Phase A: supercritical CO₂, and Phase B: MeOH (0.05% DEA); Gradient: 5~40% B) to give the 1^{st} enantiomer (7a) (165 mg, 588.06 μmol, 3.81% yield, 96% purity) and the 2^{nd} enantiomer (7b) (190 mg, 648.95 μmol, 4.21% yield, 92% purity), both as yellow oil.

### Procedure for the preparation of N-methyl-6,7-dihydro-5H-thieno[3,2-b]pyran-6-amine hydrochloride enantiomer 1 (46)

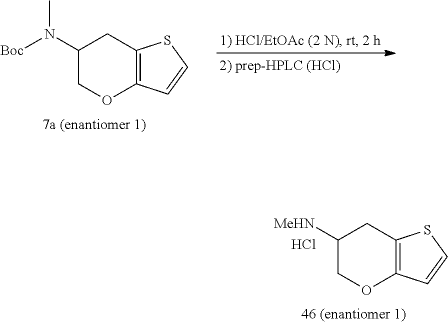 [SMILES: CN(C(=O)OC(C)(C)C)C1COc2ccsc2C1 ; Cl ; CNC1COc2ccsc2C1]

To a solution of tert-butyl N-(6,7-dihydro-5H-thieno[3,2-b]pyran-6-yl)-N-methyl-carbamate enantiomer 1 (7a) (165 mg, 613 μmol, 1 eq) in EtOAc (2 mL) was added HCl/EtOAc (4 M, 2 mL), and the resulting mixture was stirred at 25° C. for 2 hr. The reaction mixture was concentrated in vacuo and the residue was purified by prep-HPLC (Column: 3_Phenomenex Luna C18 75*30 mm*3 μm; mobile phase: [water (HCl)-ACN]; B %: 0%-6%, 8 min) to give N-methyl-6,7-dihydro-5H-thieno[3,2-b]pyran-6-amine hydrochloride salt enantiomer 1 (46) (65.47 mg, 382.97 μmol, 62.52% yield) as a white solid. ¹H NMR (400 MHz, CDCl₃) δ 10.00 (brs, 1H), 9.85 (brs, 1H), 7.06 (d, J=5.4 Hz, 1H), 6.64 (d, J=5.4 Hz, 1H), 4.52 (dd, J=6.3, 11.6 Hz, 1H), 4.38 (dd, J=1.5, 11.5 Hz, 1H), 3.68-3.64 (m, 1H), 3.26 (d, J=5.5 Hz, 2H), 2.78 (s, 3H). LCMS: m/z [M+H]⁺=170.0.

### Procedure for preparation of N-methyl-6,7-dihydro-5H-thieno[3,2-b]pyran-6-amine hydrochloride enantiomer 2 (47)

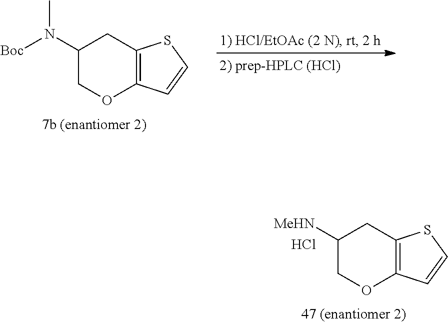 [SMILES: CN(C(=O)OC(C)(C)C)C1COc2ccsc2C1 ; Cl ; CNC1COc2ccsc2C1]

To a solution of tert-butyl N-(6,7-dihydro-5H-thieno[3,2-b]pyran-6-yl)-N-methyl-carbamate enantiomer 2 (7b) (190 mg, 705 umol, 1 eq) in EtOAc (2 mL) was added HCl/EtOAc (4 M, 2 mL), and the resulting mixture was stirred at 25° C. for 2 hr. The reaction mixture was concentrated in vacuo, and the residue was purified by prep-HPLC (column: 3_Phenomenex Luna C18 75*30 mm*3 μm; mobile phase: [water(HCl)-ACN]; B %: 0%-6%, 8 min) to give N-methyl-6,7-dihydro-5H-thieno[3,2-b]pyran-6-amine hydrochloride salt enantiomer 2 (47) (79.82 mg, 457.48 umol, 64.86% yield) as a white solid. ¹H NMR (400 MHz, CDCl3, 296 K) δ 9.96 (brs, 1H), 9.82 (brs, 1H), 7.06 (d, J=5.4 Hz, 1H), 6.63 (d, J=5.4 Hz, 1H), 4.51 (dd, J=6.3, 11.6 Hz, 1H), 4.44-4.29 (m, 1H), 3.68-3.64 (m, 1H), 3.25 (d, J=5.6 Hz, 2H), 2.78 (s, 3H). LCMS: m/z [M+H]⁺=170.0.

### Synthetic Scheme for the preparation of N, 6-dimethyl-5,7-dihydro-4H-benzothiophen-6-amine hydrochloride enantiomer 1 (49) and enantiomer 2 (50)

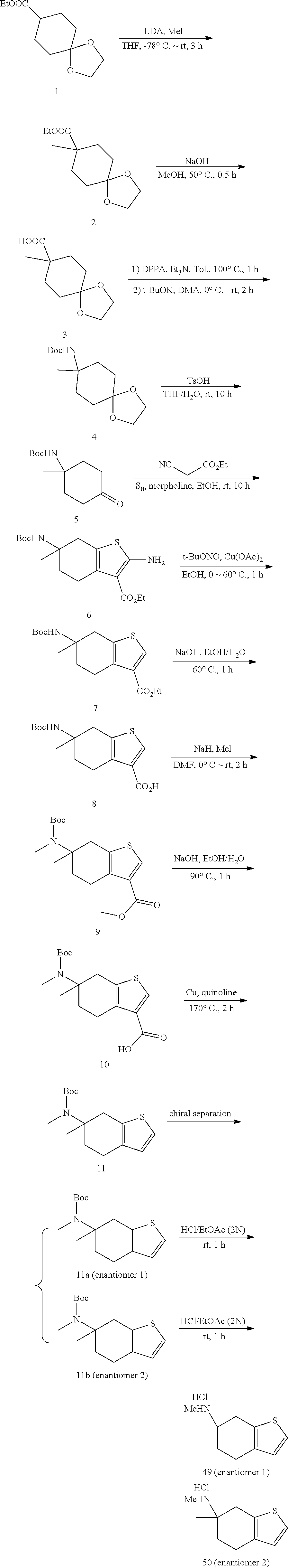

### Procedure for the preparation of ethyl 8-methyl-1,4-dioxaspiro[4.5]decane-8-carboxylate (2)

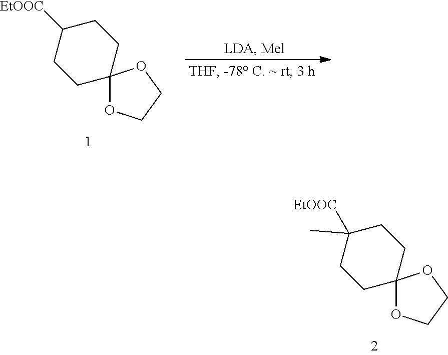 [SMILES: CCOC(=O)C1CCC2(CC1)OCCO2 ; "LDA, Mel" ; CCOC(=O)C1(C)CCC2(CC1)OCCO2]

To a solution of LDA (2 M in THF, 30.34 mL, 60.7 mmol, 1.3 eq) in THF (50 mL) was added a solution consisting of ethyl 1,4-dioxaspiro[4.5]decane-8-carboxylate (1) (10.0 g, 46.67 mmol, 1 eq) and THF (30 mL) dropwise at −78° C. under N₂ atmosphere, and the resulting mixture was stirred at that temperature for 0.5 h. A solution of CH₃I (13.25 g, 93.35 mmol, 5.81 mL, 2 eq) in THF (20 mL) was then added drop-wise, and the resultant reaction mixture was stirred at −78° C. for 1 h before being warmed slowly to −10° C. and stirred for another 1 hr. The reaction mixture was quenched with sat.NH₄Cl (100 mL) and extracted with EtOAc (100 mL*2). The combined organic layers were washed with brine (100 mL), dried over anhydrous Na₂SO₄, filtered and concentrated under reduced pressure to give ethyl 8-methyl-1,4-dioxaspiro[4.5]decane-8-carboxylate (2) (10 g, crude) as a yellowish oil. ¹H NMR (400 MHz, CHLOROFORM-d) δ 4.14 (q, J=7.1 Hz, 2H), 3.93 (s, 4H), 2.20-2.07 (m, 2H), 1.70-1.57 (m, 4H), 1.55-1.46 (m, 2H), 1.25 (t, J=7.1 Hz, 3H), 1.19 (s, 3H).

### Procedure for the preparation of 8-methyl-1, 4-dioxaspiro[4.5]decane-8-carboxylic acid (3)

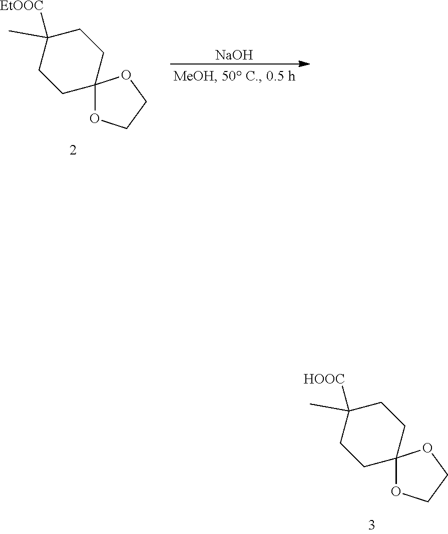 [SMILES: CCOC(=O)C1(C)CCC2(CC1)OCCO2 ; "NaOH" ; CC1(C(=O)O)CCC2(CC1)OCCO2]

To a solution of ethyl 8-methyl-1,4-dioxaspiro[4.5]decane-8-carboxylate (2) (10.0 g, 43.81 mmol, 1 eq) in MeOH (80 mL) was added 3 M aq. NaOH (44 mL), and the resulting mixture was stirred at 50° C. for 0.5 hr. The reaction mixture was cooled to rt, concentrated under reduced pressure to remove MeOH, diluted with H₂O (30 mL), treated with 3 M HCl until pH 6, and then extracted with EtOAc (100 mL*3). The combined organic layers were washed with brine (100 mL), dried over anhydrous Na₂SO₄, filtered, and concentrated under reduced pressure to give 8-methyl-1, 4-dioxaspiro[4.5]decane-8-carboxylic acid (3) (9 g, quant.) as a yellow oil. ¹H NMR (400 MHz, CDCl₃) δ 3.87 (s, 4H), 2.13-2.04 (m, 2H), 1.67-1.58 (m, 4H), 1.52-1.40 (m, 2H), 1.19 (s, 3H).

### Procedure for the preparation of tert-butyl N-(8-methyl-1,4-dioxaspiro[4.5]decan-8-yl) carbamate (4)

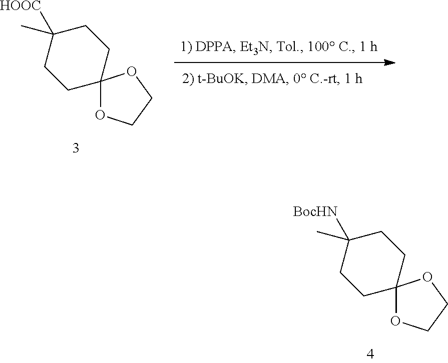

To a solution of 8-methyl-1,4-dioxaspiro[4.5]decane-8-carboxylic acid (3) (8.5 g, 42.45 mmol, 1 eq) in toluene (80 mL) were added DPPA (14.02 g, 50.94 mmol, 11.04 mL, 1.2 eq) and Et₃N (5.15 g, 50.94 mmol, 7.09 mL, 1.2 eq) under N₂ atmosphere. The resultant mixture was stirred at 100° C. for 1 hr. The mixture was cooled to rt, poured into sat.NaHCO₃ (100 mL), and extracted with EtOAc (100 mL*2). The combined organic layers were washed with brine (100 mL), dried over anhydrous Na₂SO₄, filtered and concentrated to dryness in vacuo. The residue was dissolved in DMA (60 mL), followed by the addition of t-BuOK (5.72 g, 50.94 mmol, 1.2 eq) batch-wise at 0° C., and the resulting mixture was stirred at 25° C. for 1 hr. The reaction mixture was poured into H₂O (200 mL) and extracted with EtOAc (100 mL*3). The combined organic layers were washed with brine (10 mL), dried over anhydrous Na₂SO₄, filtered and concentrated under reduced pressure to give tert-butyl N-(8-methyl-1,4-dioxaspiro[4.5]decan-8-yl)carbamate (4) (8.4 g, crude) as a yellow solid. ¹H NMR (400 MHz, CHLOROFORM-d) δ 4.39 (br s, 1H), 3.95 (s, 4H), 2.09-1.98 (m, 2H), 1.71-1.57 (m, 6H), 1.44 (s, 9H), 1.34 (s, 3H).

### Procedure for the preparation of tert-butyl N-(1-methyl-4-oxo-cyclohexyl) carbamate (5)

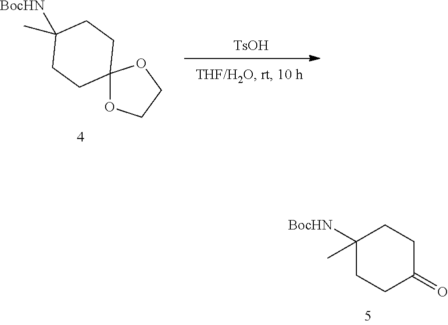 [SMILES: CC1(NC(=O)OC(C)(C)C)CCC2(CC1)OCCO2 ; "TsOH" ; CC1(NC(=O)OC(C)(C)C)CCC(=O)CC1]

To a solution of tert-butyl N-(8-methyl-1,4-dioxaspiro[4.5]decan-8-yl)carbamate (4) (8.4 g, crude) in THF (60 mL) and H₂O (60 mL) was added TsOH (10.66 g, 61.91 mmol). The resulting mixture was stirred at 25° C. for 10 hr. The reaction mixture was quenched with sat.NaHCO₃ (100 mL), and extracted with EtOAc (50 mL*3). The combined organic layers were washed with brine (50 mL), dried over anhydrous Na₂SO₄, filtered, and concentrated under reduced pressure. The residue was purified by flash silica gel chromatography (ISCO®; 40 g SepaFlash® Silica Flash Column, eluent 0~20% Ethyl acetate/Petroleum ether) to give tert-butyl N-(1-methyl-4-oxo-cyclohexyl) carbamate (5) (5.4 g, 23.76 mmol, 55.97% yield over 2 steps) as a yellow oil. ¹H NMR (400 MHz, CDCl₃) δ 4.53 (br s, 1H), 2.53-2.24 (m, 6H), 1.79 (dt, J=4.8, 12.7 Hz, 2H), 1.45 (s, 9H), 1.43 (s, 3H).

### Procedure for the preparation of ethyl 2-amino-6-(tert-butoxycarbonylamino)-6-methyl-5,7-dihydro-4H-benzothiophene-3-carboxylate (6)

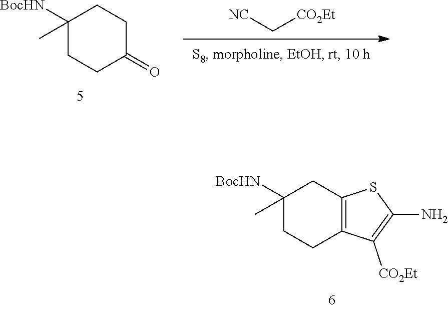 [SMILES: CC1(NC(=O)OC(C)(C)C)CCC(=O)CC1 ; CCOC(=O)CC#N ; CCOC(=O)c1c(N)sc2c1CCC(C)(NC(=O)OC(C)(C)C)C2]

To a solution of tert-butyl N-(1-methyl-4-oxo-cyclohexyl)carbamate (5) (4.4 g, 19.36 mmol, 1 eq), ethyl 2-cyanoacetate (2.41 g, 21.29 mmol, 2.27 mL, 1.1 eq) and morpholine (1.69 g, 19.36 mmol, 1.70 mL, 1 eq) in EtOH (60 mL) was added sulfur (890 mg, 27.76 mmol, 1.43 eq), and the resulting mixture was stirred at 25° C. for 10 hr. The reaction mixture was poured into H₂O (100 mL) and extracted with EtOAc (300 mL*2). The combined organic layers were washed with brine (100 mL), dried over anhydrous Na₂SO₄, filtered and concentrated under reduced pressure. The residue was purified by flash silica gel chromatography (ISCO®; 40 g SepaFlash® Silica Flash Column, eluent 0~80% Ethyl acetate/Petroleum ether) to give ethyl 2-amino-6-(tert-butoxycarbonylamino)-6-methyl-5,7-dihydro-4H-benzothiophene-3-carboxylate (6) (6 g, 16.93 mmol, 87.44% yield) as a white solid. LCMS: m/z [M+H]⁺=355.2.

### Procedure for the preparation of ethyl 6-(tert-butoxycarbonylamino)-6-methyl-5,7-dihydro-4H-benzothiophene-3-carboxylate (7)

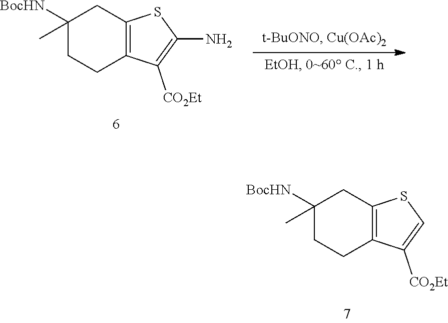 [SMILES: CCOC(=O)c1c(N)sc2c1CCC(C)(NC(=O)OC(C)(C)C)C2 ; CCOC(=O)c1csc2c1CCC(C)(NC(=O)OC(C)(C)C)C2]

To a solution of ethyl 2-amino-6-(tert-butoxycarbonylamino)-6-methyl-5,7-dihydro-4H-benzothiophene-3-carboxylate (6) (6.0 g, 16.93 mmol, 1 eq) and Cu(OAc)₂ (4.61 g, 25.39 mmol, 1.5 eq) in EtOH (80 mL) was added t-BuONO (1.92 g, 18.62 mmol, 2.21 mL, 1.1 eq) at 0° C., and the resulting mixture was stirred at 60° C. for 1 hr. The reaction mixture was cooled to rt, filtered, and the filtrate concentrated under reduced pressure. The residue was purified by flash silica gel chromatography (ISCO®; 40 g SepaFlash® Silica Flash Column, eluent 0~15% Ethyl acetate/Petroleum ether) to give ethyl 6-(tert-butoxycarbonylamino)-6-methyl-5,7-dihydro-4H-benzothiophene-3-carboxylate (7) (3 g, 8.84 mmol, 52.21% yield) as a white solid. LCMS: m/z [M+H-Boc]⁺=240.4.

### Procedure for the preparation of 6-(tert-butoxycarbonylamino)-6-methyl-5,7-dihydro-4H-benzothiophene-3-carboxylic acid (8)

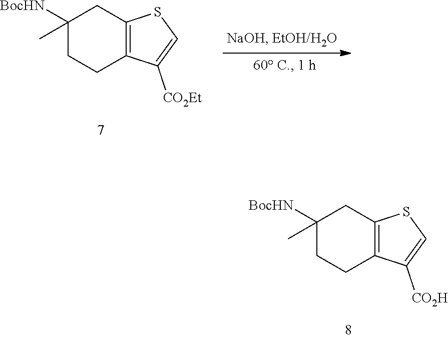 [SMILES: CCOC(=O)c1csc2c1CCC(C)(NC(=O)OC(C)(C)C)C2 ; CC1(NC(=O)OC(C)(C)C)CCc2c(C(=O)O)csc2C1]

To a solution of ethyl 6-(tert-butoxycarbonylamino)-6-methyl-5,7-dihydro-4H-benzothiophene-3-carboxylate (7) (3.0 g, 8.84 mmol, 1 eq) in EtOH (30 mL) was added a solution of NaOH (1.06 g, 26.51 mmol, 3 eq) in H₂O (15 mL), and the resulting mixture was stirred at 60° C. for 1 hr. The reaction mixture was concentrated under reduced pressure to remove EtOH, diluted with H₂O (50 mL), and treated with 1 M HCl until pH 7. The precipitate was collected by filtration, washed with H₂O, and dried under reduced pressure to give 6-(tert-butoxycarbonylamino)-6-methyl-5,7-dihydro-4H-benzothiophene-3-carboxylic acid (8) (2.7 g, 8.67 mmol, 98.11% yield) as a white solid.

### Procedure for the preparation of 6-[tert-butoxycarbonyl (methyl) amino]-6-methyl-5,7-dihydro-4H-benzothiophene-3-carboxylic acid (10)

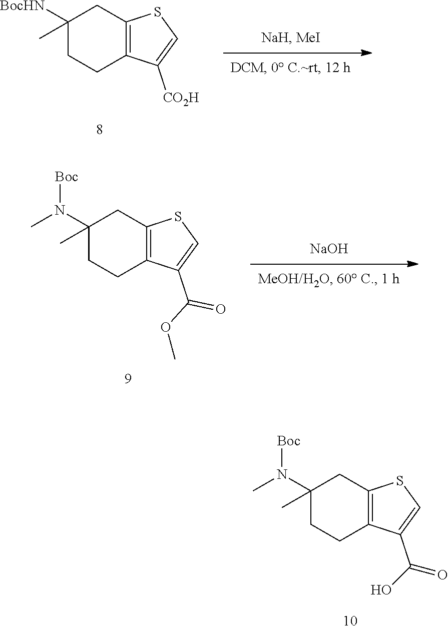 [SMILES: CC1(NC(=O)OC(C)(C)C)CCc2c(C(=O)O)csc2C1 ; COC(=O)c1csc2c1CCC(C)(N(C)C(=O)OC(C)(C)C)C2 ; "NaOH" ; CN(C(=O)OC(C)(C)C)C1(C)CCc2c(C(=O)O)csc2C1]

To a solution of 6-(tert-butoxycarbonylamino)-6-methyl-5,7-dihydro-4H-benzothiophene-3-carboxylic acid (8) (2 g, 6.42 mmol, 1 eq) in DMF (30 mL) was added NaH (1.03 g, 25.69 mmol, 60% in oil, 4 eq) batch-wise at 0° C., and the resulting mixture was stirred at 0° C. for 0.5 h. CH₃I (2.73 g, 19.27 mmol, 1.20 mL, 3 eq) was then added drop-wise, and the resulting mixture was stirred at 25° C. for 12 hr. The reaction mixture was quenched by slowly adding sat. NH₄Cl (50 mL) at 0° C., and then extracted with EtOAc (50 mL*2). The combined organic layers were washed with brine (50 mL), dried over anhydrous Na₂SO₄, filtered and concentrated under reduced pressure. The residue was dissolved in MeOH (20 mL) and H₂O (10 mL), followed by the addition of NaOH (270 mg, 6.75 mmol), and the resulting mixture was stirred at 60° C. for 1 h. The reaction mixture was cooled to rt, concentrated under reduced pressure to remove MeOH, diluted with H₂O (30 mL), treated with 1 N HCl until pH 7, and extracted with EtOAc (30 mL*2). The combined organic layers were washed with brine (40 mL), dried over anhydrous Na₂SO₄, filtered and concentrated under reduced pressure. The residue was purified by flash silica gel chromatography (ISCO®; 20 g SepaFlash® Silica Flash Column, eluent 0~25% Ethyl acetate/Petroleum ether) to give 6-[tert-butoxycarbonyl(methyl)amino]-6-methyl-5,7-dihydro-4H-benzothiophene-3-carboxylic acid (10) (1.3 g, 2.92 mmol, 45.40% yield) as a white solid. ¹H NMR (400 MHz, METHANOL-d₄) δ 8.00 (s, 1H), 3.37-3.31 (m, 1H), 2.99-2.71 (m, 7H), 1.84-1.73 (m, 1H), 1.46 (s, 9H), 1.43 (s, 3H).

### Procedure for the preparation of tert-butyl N-methyl-N-(6-methyl-5,7-dihydro-4H-benzo thiophen-6-yl) carbamate (11a, Jib)

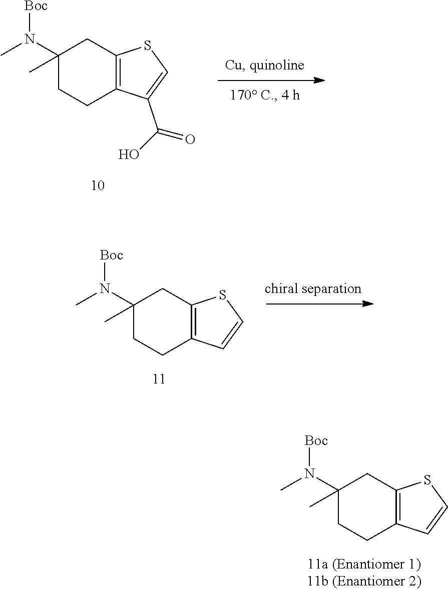 [SMILES: CN(C(=O)OC(C)(C)C)C1(C)CCc2c(C(=O)O)csc2C1 ; CN(C(=O)OC(C)(C)C)C1(C)CCc2ccsc2C1 ; CN(C(=O)OC(C)(C)C)C1(C)CCc2ccsc2C1]

To a solution of 6-[tert-butoxycarbonyl(methyl)amino]-6-methyl-5,7-dihydro-4H-benzothiophene-3-carboxylic acid (10) (0.6 g, 1.84 mmol, 1 eq) in quinoline (10 mL) was added Cu dust (234 mg, 3.69 mmol, 2 eq), and the resulting mixture was stirred at 170° C. for 4 hr. The reaction mixture was cooled to rt, diluted with EtOAc (30 mL), and washed with 0.5 M HCl (100 mL*2). The organic layer was washed with brine (10 mL), dried over anhydrous Na₂SO₄, filtered, and concentrated under reduced pressure. The residue was purified by flash silica gel chromatography (ISCO®; 40 g SepaFlash® Silica Flash Column, eluent 0~10% Ethyl acetate/Petroleum ether) to give the racemic tert-butyl N-methyl-N-(6-methyl-5,7-dihydro-4H-benzothiophen-6-yl)carbamate (11), which was further separated by prep-Chiral-SFC (column: DAICEL CHIRALPAK IC (250 mm*30 mm, 5 μm); mobile phase: [0.1% NH₃H₂O in EtOH]; B %: 15%-15%, 2.7 min) to successively give Enantiomer 1 (11a) (190 mg, 668.41 μmol, 18.13% yield) and Enantiomer 2 (11b) (190 mg, 668.41 μmol, 18.13% yield), both as yellow oil.

Enantiomer 1 (11a): ¹H NMR (400 MHz, CHLOROFORM-d) δ 7.06 (d, J=5.0 Hz, 1H), 6.76 (d, J=5.0 Hz, 1H), 3.23 (d, J=16.8 Hz, 1H), 2.90 (d, J=16.9 Hz, 1H), 2.85-2.76 (m, 4H), 2.63 (t, J=6.0 Hz, 2H), 1.77 (td, J=7.3, 13.2 Hz, 1H), 1.47 (s, 9H), 1.43 (s, 3H). LCMS: m/z [M+H-Boc]⁺=181.4.

Enantiomer 2 (11b): ¹H NMR (400 MHz, CHLOROFORM-d) δ 7.06 (d, J=5.0 Hz, 1H), 6.76 (d, J=5.0 Hz, 1H), 3.23 (d, J=16.9 Hz, 1H), 2.90 (d, J=16.8 Hz, 1H), 2.86-2.75 (m, 4H), 2.63 (t, J=6.0 Hz, 2H), 1.83-1.72 (m, 1H), 1.47 (s, 9H), 1.43 (s, 3H). LCMS: m/z [M+H-Boc]⁺=181.4.

### Procedure for the preparation of N, 6-dimethyl-5,7-dihydro-4H-benzothiophen-6-amine hydrochloride enantiomer 1 (49)

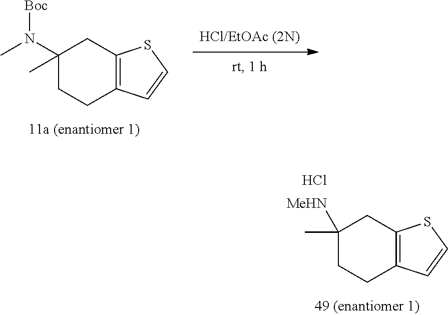 [SMILES: CN(C(=O)OC(C)(C)C)C1(C)CCc2ccsc2C1 ; Cl ; CNC1(C)CCc2ccsc2C1]

To a solution of tert-butyl N-methyl-N-(6-methyl-5,7-dihydro-4H-benzothiophen-6-yl) carbamate (11a, Enantiomer 1) (190 mg, 668 μmol, 99% purity, 1 eq) in EtOAc (2 mL) was added 4 N HCl/EtOAc (2 mL), and the resulting mixture was stirred at 25° C. for 1 hr. The precipitate was collected by filtration, washed with EtOAc, and then dried in vacuo to give N, 6-dimethyl-5,7-dihydro-4H-benzothiophen-6-amine hydrochloride salt enantiomer 1 (49) (116.77 mg, 530.87 μmol, 79.42% yield) as a white solid. ¹H NMR (400 MHz, METHANOL-d₄) δ 7.25 (d, J=5.1 Hz, 1H), 6.83 (d, J=5.1 Hz, 1H), 3.06 (s, 2H), 2.87-2.76 (m, 2H), 2.71 (s, 3H), 2.13-1.97 (m, 2H), 1.42 (s, 3H). LCMS: m/z [M+H]⁺=182.1.

### Procedure for the preparation of N, 6-dimethyl-5,7-dihydro-4H-benzothiophen-6-amine hydrochloride enantiomer 2 (50)

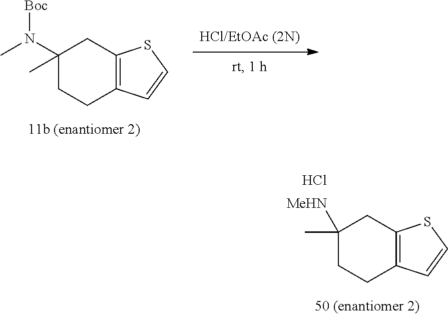 [SMILES: CN(C(=O)OC(C)(C)C)C1(C)CCc2ccsc2C1 ; Cl ; CNC1(C)CCc2ccsc2C1]

To a solution of tert-butyl N-methyl-N-(6-methyl-5,7-dihydro-4H-benzothiophen-6-yl) carbamate (11b, Enantiomer 2) (190 mg, 675 μmol, 1 eq) in EtOAc (2 mL) was added 4 N HCl/EtOAc (2 mL) and the resulting mixture was stirred at 25° C. for 1 hr. The precipitate was collected by filtration, washed with EtOAc, and then dried in vacuo to give N, 6-dimethyl-5,7-dihydro-4H-benzothiophen-6-amine hydrochloride salt enantiomer 2 (50) (109.34 mg, 497.10 μmol, 73.63% yield) as a white solid. ¹H NMR (400 MHz, METHANOL-d₄) δ 7.25 (d, J=5.1 Hz, 1H), 6.83 (d, J=5.1 Hz, 1H), 3.05 (s, 2H), 2.86-2.77 (m, 2H), 2.71 (s, 3H), 2.12-1.99 (m, 2H), 1.42 (s, 3H). LCMS: m/z [M+H]⁺=182.1.

### Synthetic Scheme for the preparation of N-methyl-3,4-dihydro-2H-thieno[3,4-b]pyran-3-amine hydrochloride enantiomer 1 (51) and enantiomer2 (52)

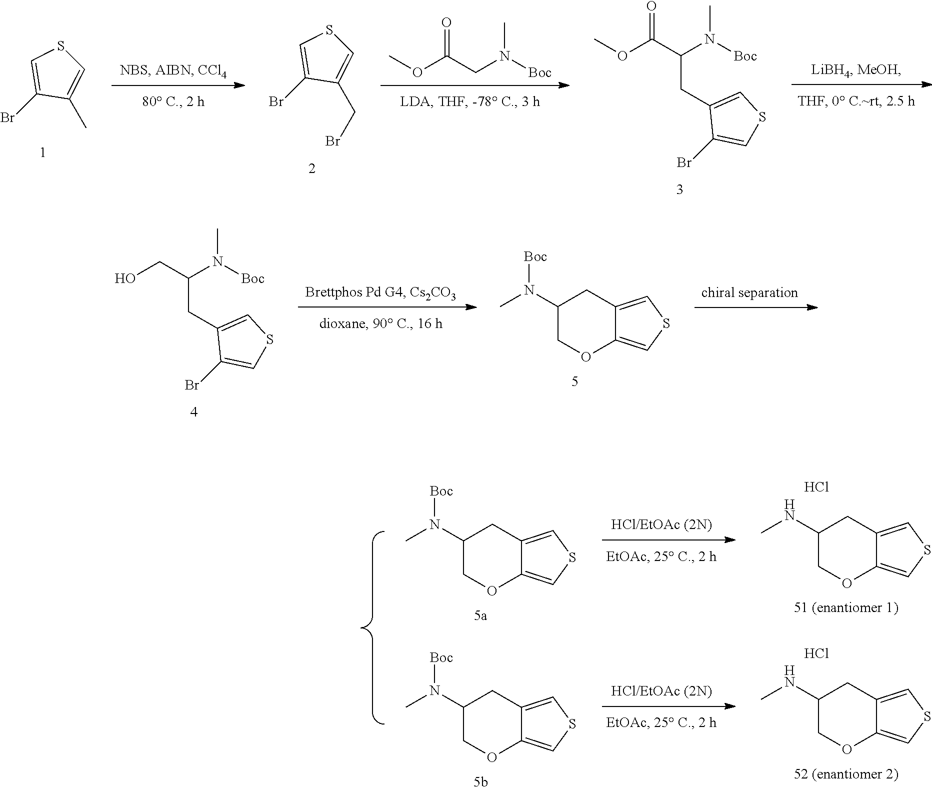

### Procedure for the preparation of 3-bromo-4-(bromomethyl)thiophene (2)

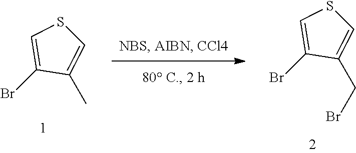

To a mixture of 3-bromo-4-methyl-thiophene (1) (10.0 g, 56.48 mmol, 1 eq) and AIBN (464 mg, 2.82 mmol, 0.05 eq) in CCl₄ (100 mL) was added NBS (10.55 g, 59.30 mmol, 1.05 eq) under N₂ atmosphere. The mixture was stirred at 80° C. for 2 hours under N₂. The reaction mixture was cooled to rt, poured into H₂O (100 mL), and extracted with DCM (50 mL*3). The combined organic phase was washed with brine, dried over Na₂SO₄, filtered, and concentrated under reduced pressure to give 3-bromo-4-(bromomethyl)thiophene (2) (14 g, crude) as a white solid.

### Procedure for the preparation of methyl 3-(4-bromo-3-thienyl)-2-[tert-butoxycarbonyl (methyl) amino]propanoate (3)

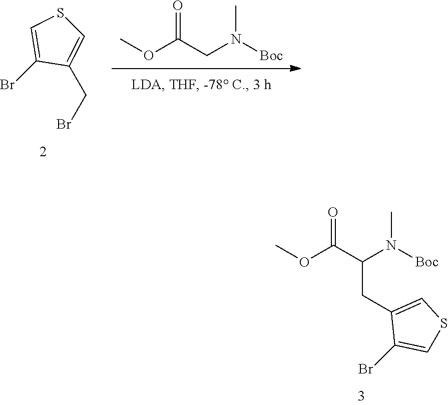

To a solution of LDA (2 M in THF, 32.8 mL, 65.6 mmol, 1.2 eq) in THF (50 mL) was added a solution of methyl 2-[tert-butoxycarbonyl(methyl)amino]acetate (13.34 g, 65.64 mmol, 1.2 eq) in THF (50 mL) drop-wise at −78° C. under N₂, and the resulting mixture was stirred for 0.5 h at that temperature. A solution consisting of 3-bromo-4-(bromomethyl)thiophene (2) (14 g, crude) and THF (50 mL) was added drop-wise, and the resulting mixture was stirred at −78° C. for 2.5 hr, slowly warmed to 0° C., and stirred for another 1 hr. The mixture was quenched with saturated ammonium chloride aqueous solution (200 mL), and extracted with EtOAc (200 mL*3). The combined organic phase was washed with brine (100 mL*3), dried over Na₂SO₄, filtered and concentrated under reduced pressure. The residue was purified by silica gel chromatography (100-200 mesh silica gel, Petroleum ether/Ethyl acetate=1/1~0/1) to afford methyl 3-(4-bromo-3-thienyl)-2-[tert-butoxycarbonyl(methyl)amino]propanoate (3) (10 g, 26.44 mmol, 46.8% yield over 2 steps) as a white solid. LCMS: m/z [M+H]⁺=278.0.

### Procedure for the preparation of tert-butyl N-[1-[(4-bromo-3-thienyl)methyl]-2-hydroxy-ethyl]-N-methyl-carbamate (4)

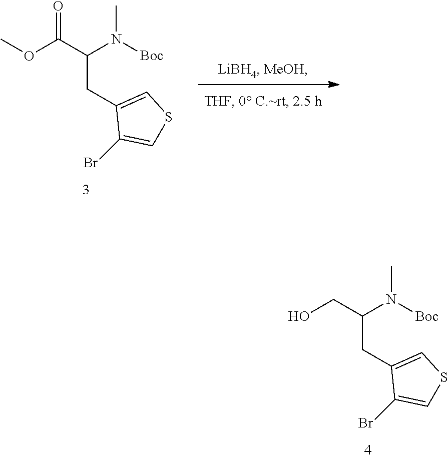 [SMILES: COC(=O)C(Cc1cscc1Br)N(C)C(=O)OC(C)(C)C ; CN(C(=O)OC(C)(C)C)C(CO)Cc1cscc1Br]

To a mixture of methyl 3-(4-bromo-3-thienyl)-2-[tert-butoxycarbonyl(methyl)amino]propanoate (3) (10 g, 26.44 mmol, 1 eq) in THF (100 mL) was added LiBH₄ (1.07 g, 49.12 mmol, 1.86 eq) at 0° C. under N₂, and the resulting mixture was stirred at 0° C. for 30 min. Then MeOH (5 mL) was added and the reaction mixture was stirred at 25° C. for 2 hours. The mixture was quenched with saturated ammonium chloride aqueous solution (100 mL) and extracted with EtOAc (100 mL*3). The combined organic phase was washed with brine (100 mL*3), dried over Na₂SO₄, filtered and concentrated under reduced pressure. The residue was purified by silica gel chromatography (100-200 mesh silica gel, Petroleum ether/Ethyl acetate=1/1) to afford tert-butyl N-[1-[(4-bromo-3-thienyl)methyl]-2-hydroxy-ethyl]-N-methyl-carbamate (4) (6.0 g, 17.13 mmol, 64.80% yield) as a yellow oil. LCMS: m/z [M+H]⁺=294.0.

### Procedure for the preparation of tert-butyl N-(3,4-dihydro-2H-thieno[3,4-b]pyran-3-yl)-N-methyl-carbamate enantiomer 1 (5a) and enantiomer 2 (5b)

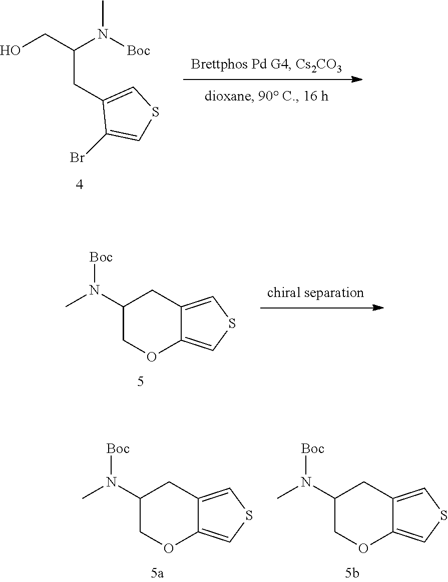

To a mixture of tert-butyl N-[1-[(4-bromo-3-thienyl)methyl]-2-hydroxy-ethyl]-N-methyl-carbamate (6.0 g, 17.13 mmol, 1 eq) and Cs₂CO₃ (16.74 g, 51.39 mmol, 3 eq) in dioxane (60 mL) was added Brettphos Pd G4 (788 mg, 856 μmol, 0.05 eq), and the resulting mixture was stirred at 90° C. for 16 hours under N₂ atmosphere. The reaction mixture was cooled to rt, filtered, and concentrated under reduced pressure. The residue was purified by silica gel chromatography (100-200 mesh silica gel, Petroleum ether/Ethyl acetate=0/1-1/1) to afford the racemic tert-butyl N-(3, 4-dihydro-2H-thieno[3,4-b]pyran-3-yl)-N-methyl-carbamate (5) (400 mg), which was further separated by prep-Chiral-SFC (column: DAICEL CHIRALPAK AD (250 mm*30 mm, 10 um); mobile phase: [0.1% NH₃H₂O in MeOH]; B %: 50%-50%, 6.3 min) to successively give the 1^{st} enantiomer (5a) (110 mg, 408.38 μmol, 2.38% yield) and the 2^{nd} enantiomer (5b) (120 mg, 445.50 μmol, 2.60% yield), both as white solids.

Enantiomer 1 (5a): ¹H NMR (400 MHz, CDCl₃) δ 6.87 (d, J=3.2 Hz, 1H), 6.35 (d, J=3.4 Hz, 1H), 4.61-4.32 (m, 1H), 4.20-4.04 (m, 2H), 3.06-2.87 (m, 2H), 2.80 (s, 3H), 1.48 (s, 9H). LCMS: m/z [M+H]⁺=214.4.

Enantiomer 2 (5b): ¹H NMR (400 MHz, CDCl₃) δ 6.87 (d, J=3.2 Hz, 1H), 6.35 (d, J=3.3 Hz, 1H), 4.61-4.29 (m, 1H), 4.22-4.02 (m, 2H), 3.09-2.88 (m, 2H), 2.80 (s, 3H), 1.48 (s, 9H). LCMS: m/z [M+H]⁺=214.4.

### Procedure for the preparation of N-methyl-3,4-dihydro-2H-thieno[3,4-b]pyran-3-amine hydrochloride enantiomer 1 (51)

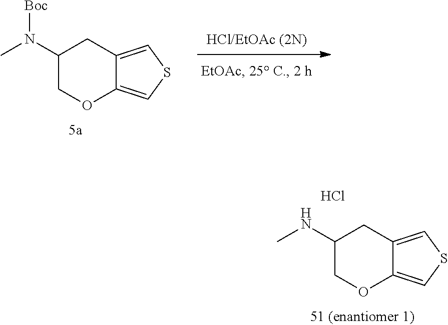 [SMILES: CN(C(=O)OC(C)(C)C)C1COc2cscc2C1 ; Cl ; CNC1COc2cscc2C1]

To a mixture of tert-butyl N-(3,4-dihydro-2H-thieno[3,4-b]pyran-3-yl)-N-methyl-carbamate enantiomer 1 (5a) (110 mg, 408 μmol, 1 eq) in EtOAc (1 mL) was added HCl/EtOAc (4 M, 1 mL), and the resulting mixture was stirred at 25° C. for 2 h. The reaction mixture was concentrated to dryness in vacuo, and the residue was purified by prep-HPLC (column: 3_Phenomenex Luna C18 75*30 mm*3 um; mobile phase: [water (HCl)-ACN]; B %: 0%-6%, 8 min) to afford N-methyl-3,4-dihydro-2H-thieno[3,4-b]pyran-3-amine hydrochloride salt enantiomer 1 (51) (52.89 mg, 312.51 μmol, 76.52% yield) as a white solid. ¹H NMR (400 MHz, Methanol-d4) δ 7.16-7.14 (m, 1H), 6.56 (d, J=3.4 Hz, 1H), 4.51-4.43 (m, 1H), 4.20 (dd, J=1.4, 12.5 Hz, 1H), 3.72-3.70 (m, 1H), 3.30-3.23 (m, 1H), 3.16-3.09 (m, 1H), 2.79 (s, 3H). LCMS: m/z [M+H]⁺=170.0.

### Procedure for the preparation of N-methyl-3,4-dihydro-2H-thieno[3,4-b]pyran-3-amine hydrochloride enantiomer 2 (52)

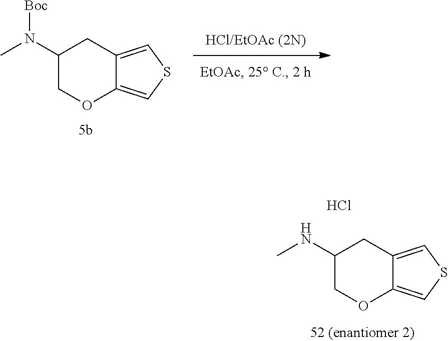 [SMILES: CN(C(=O)OC(C)(C)C)C1COc2cscc2C1 ; Cl ; CNC1COc2cscc2C1]

To a mixture of tert-butyl N-(3,4-dihydro-2H-thieno[3,4-b]pyran-3-yl)-N-methyl-carbamate enantiomer 2 (5b) (120 mg, 445.50 μmol, 1 eq) in EtOAc (1 mL) was added HCl/EtOAc (4 M, 1 mL), and the resulting mixture was stirred at 25° C. for 2 h. The reaction mixture was concentrated under reduced pressure. The residue was purified by prep-HPLC (column: 3_Phenomenex Luna C18 75*30 mm*3 um; mobile phase: [water (HCl)-ACN]; B %: 0%-6%, 8 min) to afford N-methyl-3,4-dihydro-2H-thieno[3,4-b]pyran-3-amine hydrochloride salt (52) (42.66 mg, 252.06 μmol, 56.58% yield) as a white solid. ¹H NMR (400 MHz, Methanol-d4) δ 7.18-7.13 (m, 1H), 6.56 (d, J=3.4 Hz, 1H), 4.48-4.42 (m, 1H), 4.19 (dd, J=1.4, 12.6 Hz, 1H), 3.72-3.70 (m, 1H), 3.30-3.23 (m, 1H), 3.17-3.08 (m, 1H), 2.79 (s, 3H). LCMS: m/z [M+H]⁺=170.0.

### Synthetic scheme for the preparation 3-chloro-N-methyl-4,5,6,7-tetrahydro-2-benzothiophen-5-amine hydrochloride enantiomer 1 (55) and enantiomer 2 (56) and, 1-chloro-N-methyl-4,5,6,7-tetrahydro-2-benzothiophen-5-amine hydrochloride enantiomer 1 (57) and enantiomer 2 (58)

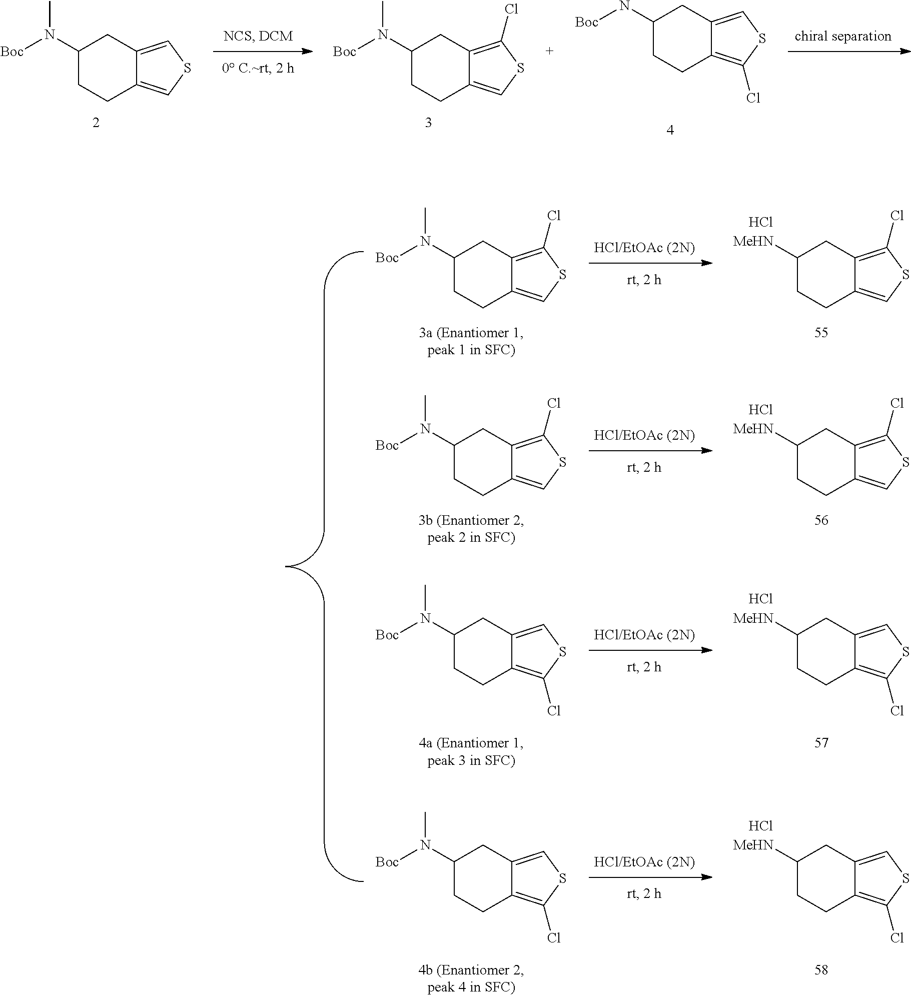

### Procedure for the preparation of tert-butyl N-(3-chloro-4,5,6,7-tetrahydro-2-benzo thiophen-5-yl)-N-methylcarbamate (3a, 3b), tert-butyl N-(1-chloro-4,5,6,7-tetrahydro-2-benzothiophen-5-yl)-N-methylcarbamate (4a, 4b)

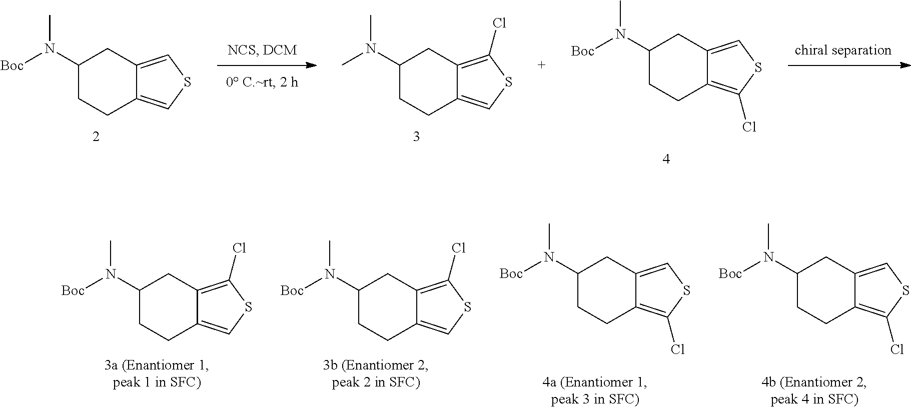

To a solution of tert-butyl N-methyl-N-(4,5,6,7-tetrahydro-2-benzothiophen-5-yl)carbamate (2) (1.8 g, 6.73 mmol, 1 eq) in DCM (20 mL) was added NCS (989 mg, 7.41 mmol, 1.1 eq) batch-wise at 0° C., and the resulting mixture was stirred at 25° C. for 2 h. The reaction mixture was poured into H₂O (10 mL) and extracted with EtOAc (40 mL*2). The combined organic phase was washed with brine (10 mL), dried over anhydrous sodium sulfate, filtered, and concentrated under vacuum. The residue was purified by prep-HPLC (column: Waters Xbridge C18 150*50 mm*10 μm; mobile phase: [water(NH₄HCO₃)-ACN]; B %: 57%-87%, 10 min) to give a racemic regioisomer mixture of N-(3-chloro-4,5,6,7-tetrahydro-2-benzothiophen-5-yl)-N-methyl-carbamate (3) and N-(1-chloro-4,5,6,7-tetrahydro-2-benzothiophen-5-yl)-N-methyl-carbamate (4). The mixture (500 mg) was subjected to prep-Chiral-SFC (column: DAICEL CHIRALPAK IG (250 mm*30 mm, 10 um); mobile phase: [0.1% NH₃H₂O in EtOH]; B %: isocratic 15% for 3.9 min) to successively afford tert-butyl N-(3-chloro-4,5,6,7-tetrahydro-2-benzothiophen-5-yl)-N-methyl-carbamate (3a, enantiomer 1, 1^{st} peak in SFC) (100 mg, 327 umol, 4.86% yield), N-(3-chloro-4,5,6,7-tetrahydro-2-benzothiophen-5-yl)-N-methyl-carbamate (3b, enantiomer 2, 2^{nd} peak in SFC) (100 mg, 328 umol, 4.87% yield), N-(1-chloro-4,5,6,7-tetrahydro-2-benzothiophen-5-yl)-N-methyl-carbamate (4a, enantiomer 1, 3^{rd} peak in SFC) (110 mg, 359 umol, 5.34% yield), N-(1-chloro-4,5,6,7-tetrahydro-2-benzothiophen-5-yl)-N-methyl-carbamate (4b, enantiomer 2, 4^{th} peak in SFC) (135 mg, 436 umol, 6.49% yield), all as yellow oil. The structures of the regioisomers were confirmed by 2D NMR.

3a (enantiomer 1, 1^{st} peak in SFC): ¹H NMR (400 MHz, CDCl₃) δ 6.68 (s, 1H), 4.57-4.05 (m, 1H), 2.99-2.63 (m, 6H), 2.50 (dd, J=12.0, 16.1 Hz, 1H), 1.99-1.69 (m, 2H), 1.49 (s, 9H).

3b (enantiomer 2, 2^{nd} peak in SFC): ¹H NMR (400 MHz, Methanol-d4) δ 6.84 (s, 1H), 4.32-4.14 (m, 1H), 2.91 (td, J=3.6, 16.5 Hz, 1H), 2.82 (s, 3H), 2.80-2.63 (m, 2H), 2.56 (dd, J=11.6, 16.0 Hz, 1H), 1.92-1.79 (m, 2H), 1.48 (s, 9H).

4a (enantiomer 1, 3^{rd} peak in SFC): ¹H NMR (400 MHz, CDCl₃) δ 6.69 (s, 1H), 4.53-4.03 (m, 1H), 2.95-2.75 (m, 5H), 2.73-2.48 (m, 2H), 1.95-1.91 (m, 1H), 1.88-1.71 (m, 1H), 1.48 (s, 9H).

4b (enantiomer 2, 4^{th} peak in SFC): ¹H NMR (400 MHz, Methanol-d4) δ 6.86 (s, 1H), 4.27-4.09 (m, 1H), 2.94-2.68 (m, 6H), 2.61-2.44 (m, 1H), 1.98-1.81 (m, 2H), 1.47 (s, 9H).

### Procedure for the preparation of 3-chloro-N-methyl-4,5,6,7-tetrahydro-2-benzothiophen-5-amine hydrochloride enantiomer 1 (55)

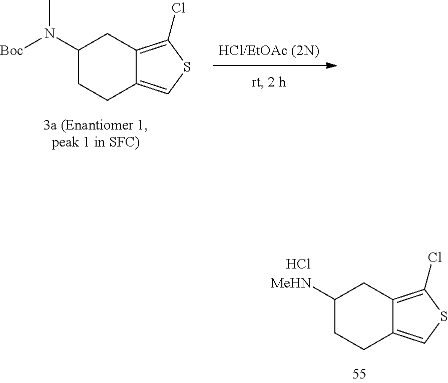 [SMILES: CN(C(=O)OC(C)(C)C)C1CCc2csc(Cl)c2C1 ; Cl ; CNC1CCc2csc(Cl)c2C1]

To a solution of tert-butyl N-(3-chloro-4,5,6,7-tetrahydro-2-benzothiophen-5-yl)-N-methyl-carbamate (3a, enantiomer 1) (100 mg, 331 eq) in EtOAc (2 mL) was added HCl/EtOAc (4 M, 2 mL). The mixture was stirred at 25° C. for 2 h. The reaction mixture was concentrated in vacuo and the residue was purified by trituration with EtOAc to give 3-chloro-N-methyl-4,5,6,7-tetrahydro-2-benzothiophen-5-amine hydrochloride enantiomer 1 (55) (66.43 mg, 84.2% yield) as a white solid. ¹H NMR (400 MHz, Methanol-d4) δ (ppm)=6.96 (s, 1H), 3.54-3.44 (m, 1H), 3.22 (ddd, J=1.6, 5.6, 16.1 Hz, 1H), 3.00-2.89 (m, 1H), 2.85-2.70 (m, 4H), 2.59 (dd, J=9.9, 16.2 Hz, 1H), 2.29-2.22 (m, 1H), 1.82 (dtd, J=5.3, 11.1, 12.8 Hz, 1H). LCMS: m/z [M+H]⁺=202.1.

### Procedure for the preparation of 3-chloro-N-methyl-4,5,6,7-tetrahydro-2-benzothiophen-5-amine hydrochloride enantiomer 2 (56)

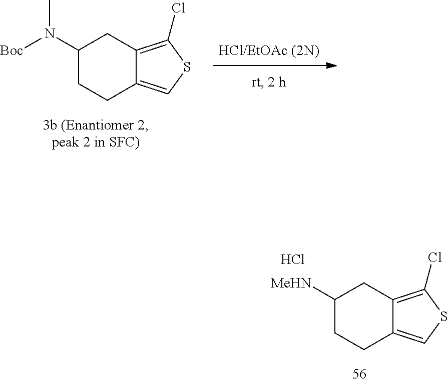 [SMILES: CN(C(=O)OC(C)(C)C)C1CCc2csc(Cl)c2C1 ; Cl ; CNC1CCc2csc(Cl)c2C1]

To a solution of tert-butyl N-(3-chloro-4,5,6,7-tetrahydro-2-benzothiophen-5-yl)-N-methyl-carbamate (3b enantiomer 2) (100 mg, 331 umol, 1 eq) in EtOAc (2 mL) was added HCl/EtOAc (4 M, 2 mL). The mixture was stirred at 25° C. for 2 h. The reaction mixture was concentrated in vacuo and the residue was purified by trituration with EtOAc to give 3-chloro-N-methyl-4,5,6,7-tetrahydro-2-benzothiophen-5-amine hydrochloride enantiomer 2 (56) (64.14 mg, 81.3% yield) as a white solid. ¹H NMR (400 MHz, Methanol-d4) δ (ppm)=6.98 (s, 1H), 3.51 (ddt, J=2.8, 5.6, 10.4 Hz, 1H), 3.24 (ddd, J=1.5, 5.5, 16.2 Hz, 1H), 2.96 (td, J=4.7, 16.9 Hz, 1H), 2.89-2.70 (m, 4H), 2.61 (dd, J=10.0, 16.1 Hz, 1H), 2.34-2.22 (m, 1H), 1.84 (dtd, J=5.3, 11.1, 12.8 Hz, 1H) LCMS: m/z [M+H]⁺=202.1.

### Procedure for the preparation of 1-chloro-N-methyl-4,5,6,7-tetrahydro-2-benzothiophen-5-amine hydrochloride enantiomer 1 (57)

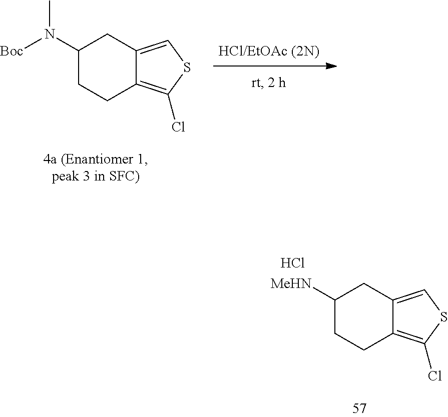 [SMILES: CN(C(=O)OC(C)(C)C)C1CCc2c(csc2Cl)C1 ; Cl ; CNC1CCc2c(csc2Cl)C1]

To a solution of tert-butyl N-(1-chloro-4,5,6,7-tetrahydro-2-benzothiophen-5-yl)-N-methyl-carbamate (4a enantiomer 1) (110 mg, 364 umol, 1 eq) in EtOAc (2 mL) was added HCl/EtOAc (4 M, 2 mL). The mixture was stirred at 25° C. for 2 h. The reaction mixture was concentrated in vacuo and the residue was purified by trituration with EtOAc to give 1-chloro-N-methyl-4,5,6,7-tetrahydro-2-benzothiophen-5-amine hydrochloride enantiomer 1 (57) (71.38 mg, 82.23% yield) as a white solid. ¹H NMR (400 MHz, Methanol-d4) δ (ppm)=7.05 (s, 1H), 3.49-3.41 (m, 1H), 3.25 (dd, J=5.2, 15.6 Hz, 1H), 2.92-2.83 (m, 1H), 2.82-2.63 (m, 5H), 2.40-2.27 (m, 1H), 1.90 (dtd, J=5.9, 10.6, 13.0 Hz, 1H). LCMS: m/z [M+H]⁺=202.1.

### Procedure for the preparation of 1-chloro-N-methyl-4,5,6,7-tetrahydro-2-benzothiophen-5-amine hydrochloride enantiomer 2 (58)

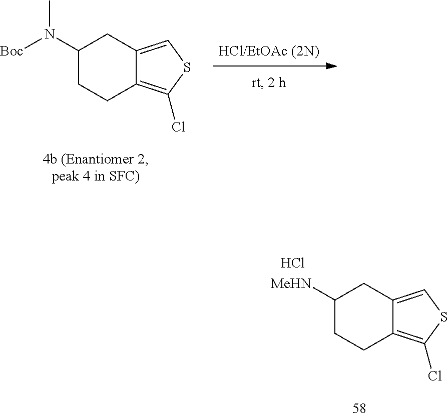 [SMILES: CN(C(=O)OC(C)(C)C)C1CCc2c(csc2Cl)C1 ; Cl ; CNC1CCc2c(csc2Cl)C1]

To a solution of tert-butyl N-(1-chloro-4,5,6,7-tetrahydro-2-benzothiophen-5-yl)-N-methyl-carbamate enantiomer 2 (4b) (135 mg, 447 umol, 1 eq) in EtOAc (2 mL) was added HCl/EtOAc (4 M, 2 mL). The mixture was stirred at 25° C. for 2 h. The reaction mixture was concentrated in vacuo and the residue was purified by trituration with EtOAc to give 1-chloro-N-methyl-4,5,6,7-tetrahydro-2-benzothiophen-5-amine hydrochloride enantiomer 2 (58) (87.54 mg, 82.17% yield) as a white solid. ¹H NMR (400 MHz, Methanol-d4) δ (ppm)=7.03 (s, 1H), 3.44 (ddt, J=2.8, 5.0, 10.2 Hz, 1H), 3.25 (dd, J=5.2, 15.6 Hz, 1H), 2.89-2.80 (m, 1H), 2.78 (s, 3H), 2.72-2.58 (m, 2H), 2.35-2.24 (m, 1H), 1.88 (dtd, J=5.9, 10.6, 13.0 Hz, 1H). LCMS: m/z [M+H]⁺=202.1.

### Synthetic scheme for the preparation of 1,3-dichloro-N-methyl-4,5,6,7-tetrahydro-2-benzothiophen-5-amine hydrochloride enantiomer 1 (59) and enantiomer 2 (60)

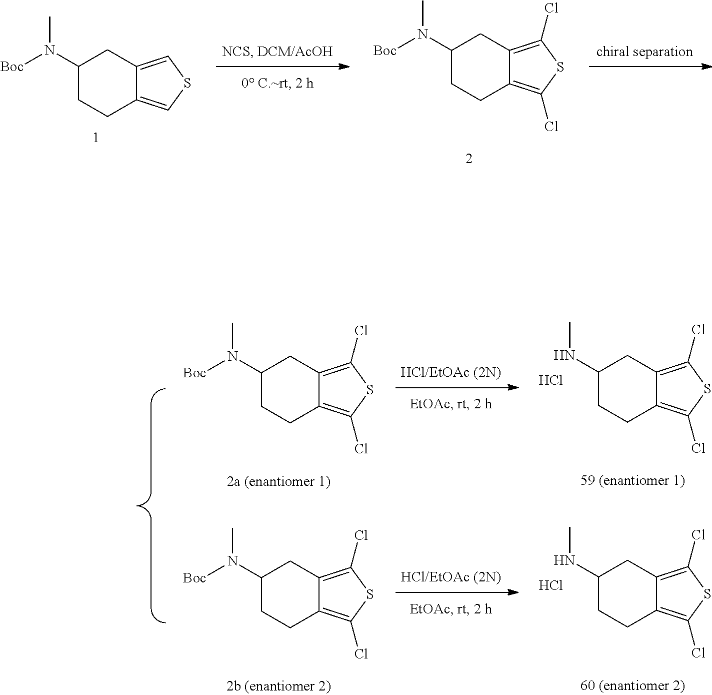

### Procedure for the preparation of tert-butyl N-(1,3-dichloro-4,5,6,7-tetrahydro-2-benzothiophen-5-yl)-N-methyl-carbamate (2a, 2b)

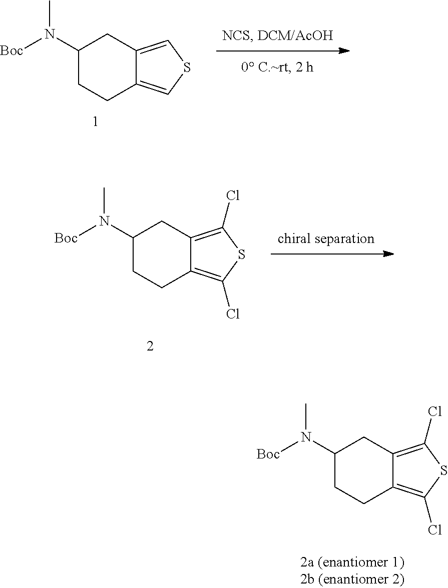 [SMILES: CN(C(=O)OC(C)(C)C)C1CCc2cscc2C1 ; CN(C(=O)OC(C)(C)C)C1CCc2c(Cl)sc(Cl)c2C1 ; CN(C(=O)OC(C)(C)C)C1CCc2c(Cl)sc(Cl)c2C1]

To a solution of tert-butyl N-methyl-N-(4,5,6,7-tetrahydro-2-benzothiophen-5-yl)carbamate (1) (500 mg, 1.87 mmol, 1 eq) in DCM (5 mL) and AcOH (1 mL) was added NCS (549 mg, 4.11 mmol, 2.2 eq) at 0° C., and the resulting mixture was stirred at 25° C. for 2 h. The reaction mixture was poured into sat. NaHCO₃ (20 mL) and extracted with EtOAc (10 mL*2). The combined organic phase was washed with brine (10 mL), dried over anhydrous sodium sulfate, filtered and concentrated under vacuum. The residue was purified by prep-HPLC (column: Waters Xbridge C18 150*50 mm*10 μm; mobile phase: [water (NH₄HCO₃)-ACN]; B %: 70%-100%, 10 min) to give the racemate tert-butyl N-(1,3-dichloro-4,5,6,7-tetrahydro-2-benzothiophen-5-yl)-N-methylcarbamate (2) (160 mg, 25.4% yield) as a yellow oil.

740 mg of the racemate was subjected to prep-Chiral-SFC (column: DAICEL CHIRALPAK AD (250 mm*30 mm, 10 μm); mobile phase: [0.1% NH₃H₂O in IPA]; B %: 15%-15%, 1.76 min) to successively afford enantiomer 1 (2a) (350 mg) and enantiomer 2 (2b) (390 mg).

### Procedure for the preparation of 1,3-dichloro-N-methyl-4,5,6,7-tetrahydro-2-benzothiophen-5-amine hydrochloride enantiomer 1 (59)

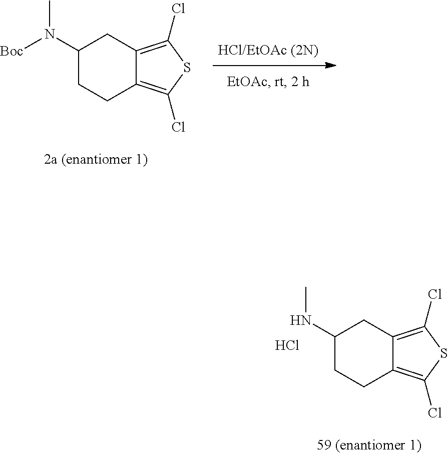 [SMILES: CN(C(=O)OC(C)(C)C)C1CCc2c(Cl)sc(Cl)c2C1 ; Cl ; CNC1CCc2c(Cl)sc(Cl)c2C1]

To a solution of tert-butyl N-(1,3-dichloro-4,5,6,7-tetrahydro-2-benzothiophen-5-yl)-N-methylcarbamate (2a, enantiomer 1) (350 mg, 1.04 mmol, 1 eq) in EtOAc (5 mL) was added HCl/EtOAc (4 M, 5 mL), and the resulting mixture was stirred at 25° C. for 2 h. The reaction mixture was concentrated under reduced pressure, and the residue was purified by trituration in EtOAc to afford 1,3-dichloro-N-methyl-4,5,6,7-tetrahydro-2-benzothiophen-5-amine hydrochloride enantiomer 1 (59) (230.34 mg, 81.18% yield) as a white solid. ¹H NMR (400 MHz, Methanol-d4) δ (ppm)=3.48 (dddd, J=2.9, 5.4, 7.8, 12.7 Hz, 1H), 3.19 (ddd, J=1.6, 5.3, 16.2 Hz, 1H), 2.92-2.83 (m, 1H), 2.80 (s, 3H), 2.68-2.54 (m, 2H), 2.34-2.24 (m, 1H), 1.84 (dtd, J=5.6, 10.8, 13.0 Hz, 1H). LCMS: m/z [M+H]⁺=236.0.

### Procedure for the preparation of 1,3-dichloro-N-methyl-4,5,6,7-tetrahydro-2-benzothiophen-5-amine hydrochloride enantiomer 2 (60)

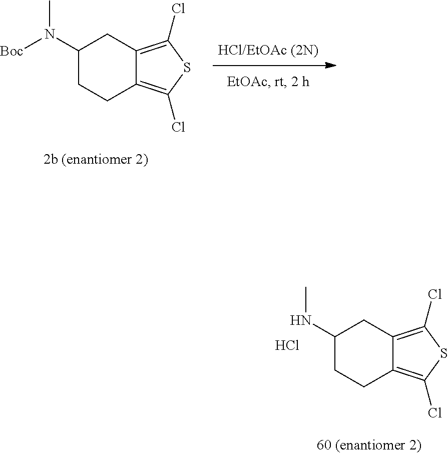 [SMILES: CN(C(=O)OC(C)(C)C)C1CCc2c(Cl)sc(Cl)c2C1 ; Cl ; CNC1CCc2c(Cl)sc(Cl)c2C1]

To a solution of tert-butyl N-(1,3-dichloro-4,5,6,7-tetrahydro-2-benzothiophen-5-yl)-N-methylcarbamate (2b, enantiomer 2) (390 mg, 1.16 mmol, 1 eq) in EtOAc (5 mL) was added HCl/EtOAc (4 M, 5 mL), and the resulting mixture was stirred at 25° C. for 2 h. The precipitate was collected by filtration, washed with EtOAc, and then dried in vacuo to give 1,3-dichloro-N-methyl-4,5,6,7-tetrahydro-2-benzothiophen-5-amine hydrochloride enantiomer 2 (60) (241.51 mg, 76.38% yield) as a white solid. ¹H NMR (400 MHz, Methanol-d4) δ (ppm)=3.53-3.43 (m, 1H), 3.20 (dd, J=4.8, 16.2 Hz, 1H), 2.88 (td, J=4.9, 17.2 Hz, 1H), 2.80 (s, 3H), 2.68-2.53 (m, 2H), 2.34-2.24 (m, 1H), 1.92-1.77 (m, 1H). LCMS: m/z [M+H]⁺=236.0.

### Synthetic scheme for the preparation of N-methyl-3-(trifluoromethyl)-4,5,6,7-tetrahydro-2-benzothiophen-5-amine hydrochloride enantiomer 1 (61) and enantiomer 2 (62), and N-methyl-1-(trifluoromethyl)-4,5,6,7-tetrahydro-2-benzothiophen-5-amine hydrochloride enantiomer 1 (63) and enantiomer 2 (64)

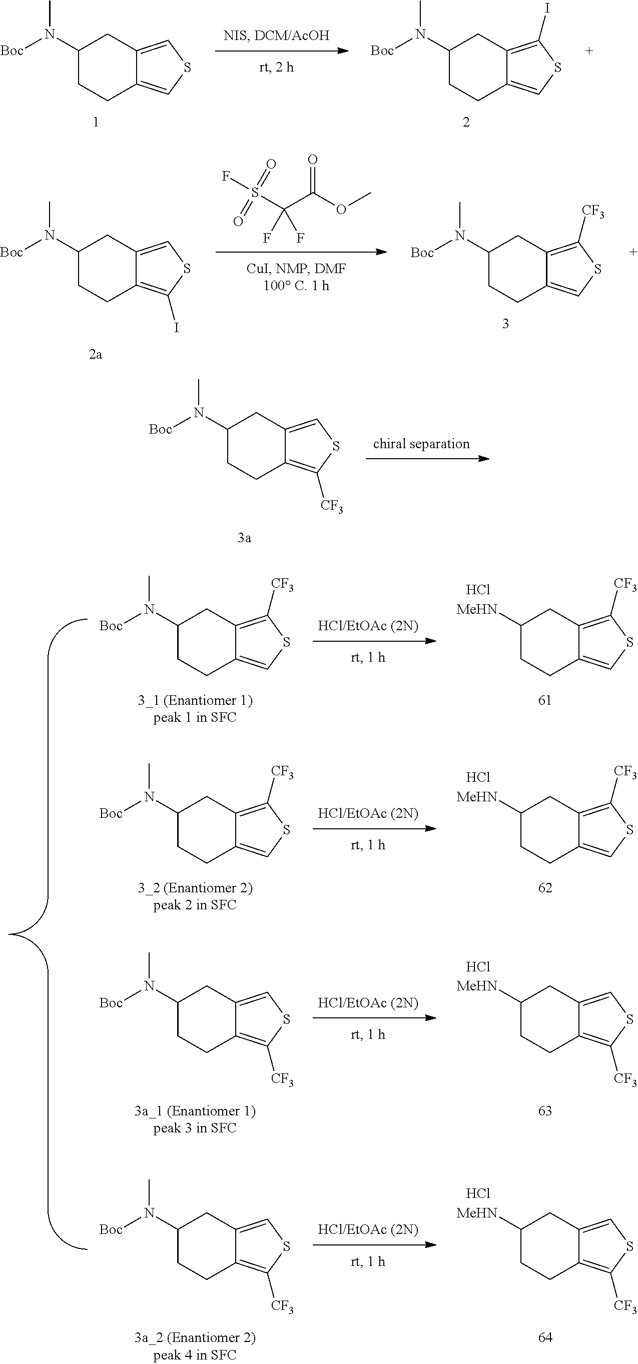

### Procedure for the preparation of tert-butyl N-(3-iodo-4,5,6,7-tetrahydro-2-benzothiophen-5-yl)-N-methylcarbamate (2) tert-butyl N-(1-iodo-4,5,6,7-tetrahydro-2-benzothiophen-5-yl)-N-methylcarbamate (2a)

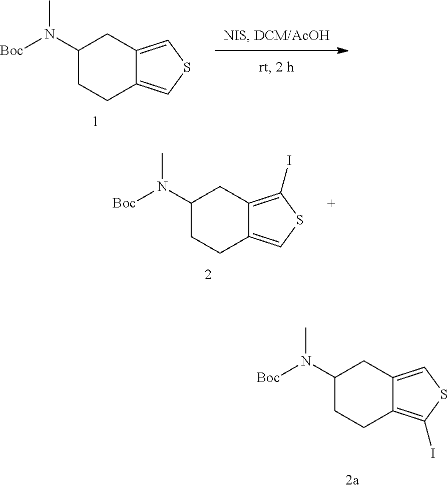 [SMILES: CN(C(=O)OC(C)(C)C)C1CCc2cscc2C1 ; CN(C(=O)OC(C)(C)C)C1CCc2csc(I)c2C1 ; CN(C(=O)OC(C)(C)C)C1CCc2c(csc2I)C1]

To a solution of tert-butyl N-methyl-N-(4,5,6,7-tetrahydro-2-benzothiophen-5-yl)carbamate (1) (2.0 g, 7.48 mmol, 1 eq) in DCM (10 mL) and AcOH (0.5 mL) was added NIS (1.51 g, 6.73 mmol, 0.9 eq) batch-wise at 0° C., and the resulting mixture was stirred at 25° C. for 2 h. The reaction mixture was poured into H₂O (10 mL) and extracted with EtOAc (30 mL*2). The combined organic phase was washed with brine (10 mL), dried over anhydrous sodium sulfate, filtered and concentrated under vacuum. The residue was purified by flash silica gel chromatography (ISCO®; 20 g SepaFlash® Silica Flash Column, eluent 0-20% Ethyl acetate/Petroleum ether) to give a mixture of tert-butyl N-(3-iodo-4,5,6,7-tetrahydro-2-benzothiophen-5-yl)-N-methyl-carbamate (2) and tert-butyl N-(1-iodo-4,5,6,7-tetrahydro-2-benzothiophen-5-yl)-N-methyl-carbamate (2a) (1.55 g, 2.33 mmol, 52.7% yield) as a yellow oil.

### Procedure for the preparation of tert-butyl N-methyl-N-[3-(trifluoromethyl)-4,5,6,7-tetrahydro-2-benzothiophen-5-yl]carbamate (3_1, 3_2), and tert-butyl N-methyl-N-[1-(trifluoromethyl)-4,5,6,7-tetrahydro-2-benzothiophen-5-yl]carbamate (3a_, 3a_2)

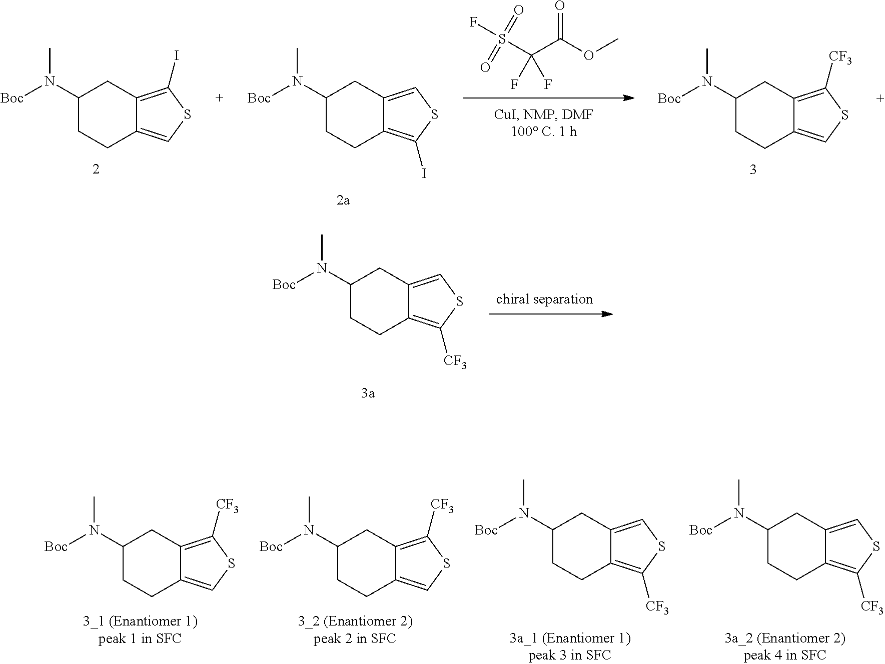

To a solution of the mixture (1.5 g, 3.81 mmol, 1 eq) of tert-butyl N-(3-iodo-4,5,6,7-tetrahydro-2-benzothiophen-5-yl)-N-methyl-carbamate (2) and tert-butyl N-(1-iodo-4,5,6,7-tetrahydro-2-benzothiophen-5-yl)-N-methyl-carbamate (2a) in DMF (10 mL) were added CuI (798 mg, 4.19 mmol, 1.1 eq) and NMP (1.89 g, 19.0 mmol, 1.85 mL, 5 eq). The resulting mixture was stirred for 10 min before the addition of methyl 2,2-difluoro-2-fluorosulfonyl-acetate (3.66 g, 19.0 mmol, 2.42 mL, 5 eq), and the stirring was continued at 100° C. for 1 h under N₂ atmosphere. The reaction mixture was cooled to rt, poured into H₂O (10 mL) and extracted with EtOAc (20 mL*2). The combined organic phase was washed with brine (10 mL), dried over anhydrous sodium sulfate, filtered and concentrated under vacuum. The residue was purified by prep-HPLC (column: Waters Xbridge C18 150*50 mm*10 μm; mobile phase: [water (NH₄HCO₃)-ACN]; B %: 57%-87%, 10 min) to give a racemic regioisomeric mixture of tert-butyl N-methyl-N-[3-(trifluoromethyl)-4,5,6,7-tetrahydro-2-benzothiophen-5-yl]carbamate (3) and N-methyl-N-[1-(trifluoromethyl)-4,5,6,7-tetrahydro-2-benzothiophen-5-yl]carbamate (3a). The mixture was subjected to prep-Chiral-SFC (column: DAICEL CHIRALPAK IG (column: DAICEL CHIRALPAK IG 250 mm*30 mm*5 μm; mobile phase: [0.1% NH₃H₂O in MeOH]; B %: 15%-15%, 1.7 min) to successively afford tert-butyl N-methyl-N-[3-(trifluoromethyl)-4,5,6,7-tetrahydro-2-benzothiophen-5-yl]carbamate (3_1 enantiomer 1) (100 mg, 7.8% yield), tert-butyl N-methyl-N-[3-(trifluoromethyl)-4,5,6,7-tetrahydro-2-benzothiophen-5-yl]carbamate (3_2 enantiomer 2) (100 mg, 7.8% yield) and tert-butyl N-methyl-N-[1-(trifluoromethyl)-4,5,6,7-tetrahydro-2-benzothiophen-5-yl]carbamate (3a_1 enantiomer 1) (100 mg, 7.8% yield), tert-butyl N-methyl-N-[1-(trifluoromethyl)-4,5,6,7-tetrahydro-2-benzothiophen-5-yl]carbamate (3a_2 enantiomer 2) (100 mg, 7.8% yield), all as yellow oil. The structures of the regioisomers were confirmed by 2D NMR of the final targets.

3_1 (enantiomer 1): ¹H NMR (400 MHz, CDCl₃) δ 7.02 (s, 1H), 4.59-4.12 (m, 1H), 3.09-3.03 (m, 1H), 2.97-2.91 (m, 1H), 2.87-2.63 (m, 5H), 1.99-1.74 (m, 2H), 1.49 (s, 9H).

3_2 (enantiomer 2): ¹H NMR (400 MHz, CDCl₃) δ 7.02 (s, 1H), 4.65-4.06 (m, 1H), 3.10-3.03 (m, 1H), 2.97-2.92 (m, 1H), 2.86-2.68 (m, 5H), 1.97-1.77 (m, 2H), 1.48 (s, 9H).

3a_1 (enantiomer 1): ¹H NMR (400 MHz, CDCl₃) δ 7.03 (s, 1H), 4.52-4.08 (m, 1H), 3.14 (td, J=2.4, 17.3 Hz, 1H), 2.97-2.64 (m, 6H), 2.07-1.76 (m, 2H), 1.48 (s, 9H).

3a_2 (enantiomer 2): ¹H NMR (400 MHz, CDCl₃) δ 7.03 (s, 1H), 4.56-4.04 (m, 1H), 3.22-3.08 (m, 1H), 2.98-2.62 (m, 6H), 2.02-1.74 (m, 2H), 1.48 (s, 9H).

### Procedure for the preparation of N-methyl-3-(trifluoromethyl)-4,5,6,7-tetrahydro-2-benzothiophen-5-amine hydrochloride enantiomer 1 (61)

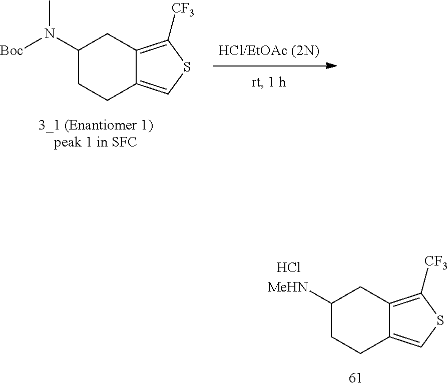 [SMILES: CN(C(=O)OC(C)(C)C)C1CCc2csc(C(F)(F)F)c2C1 ; Cl ; CNC1CCc2csc(C(F)(F)F)c2C1]

To a solution of tert-butyl N-methyl-N-[3-(trifluoromethyl)-4,5,6,7-tetrahydro-2-benzothiophen-5-yl]carbamate (3_1 enantiomer 1) (100 mg, 298 μmol, 1 eq) in EtOAc (2 mL) was added HCl/EtOAc (4 M, 2 mL), and the resulting mixture was stirred at 25° C. for 1 hr. The precipitate was collected by filtration, washed with EtOAc and dried in vacuo to give N-methyl-3-(trifluoromethyl)-4,5,6,7-tetrahydro-2-benzothiophen-5-amine hydrochloride enantiomer 1 (61) (43.67 mg, 53.90% yield) as a white solid. ¹H NMR (400 MHz, Methanol-d4) δ 7.39 (s, 1H), 3.59-3.49 (m, 1H), 3.47-3.42 (m, 1H), 3.07-2.97 (m, 1H), 2.92-2.76 (m, 5H), 2.36-2.26 (m, 1H), 1.86 (dtd, J=5.4, 11.0, 12.8 Hz, 1H). LCMS: m/z [M+H]⁺=236.0.

### Procedure for preparation of N-methyl-3-(trifluoromethyl)-4,5,6,7-tetrahydro-2-benzothiophen-5-amine hydrochloride enantiomer 2 (62)

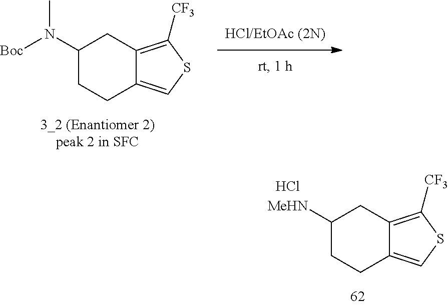 [SMILES: CN(C(=O)OC(C)(C)C)C1CCc2csc(C(F)(F)F)c2C1 ; Cl ; CNC1CCc2csc(C(F)(F)F)c2C1]

To a solution of tert-butyl N-methyl-N-[3-(trifluoromethyl)-4,5,6,7-tetrahydro-2-benzothiophen-5-yl]carbamate (3_2 enantiomer 2) (100 mg, 298 μmol, 1 eq) in EtOAc (2 mL) was added HCl/EtOAc (4 M, 2 mL). The mixture was stirred at 25° C. for 1 h. The precipitate was collected by filtration, washed with EtOAc and dried in vacuo to give N-methyl-3-(trifluoromethyl)-4,5,6,7-tetrahydro-2-benzothiophen-5-amine hydrochloride salt enantiomer 2 (62) (42.41 mg, 52.34% yield) as a white solid. ¹H NMR (400 MHz, Methanol-d4) δ 7.39 (s, 1H), 3.59-3.49 (m, 1H), 3.48-3.39 (m, 1H), 3.10-2.97 (m, 1H), 2.91-2.74 (m, 5H), 2.35-2.24 (m, 1H), 1.93-1.78 (m, 1H). LCMS: m/z [M+H]⁺=236.0.

### Procedure for the preparation of N-methyl-1-(trifluoromethyl)-4,5,6,7-tetrahydro-2-benzothiophen-5-amine hydrochloride enantiomer 1 (63)

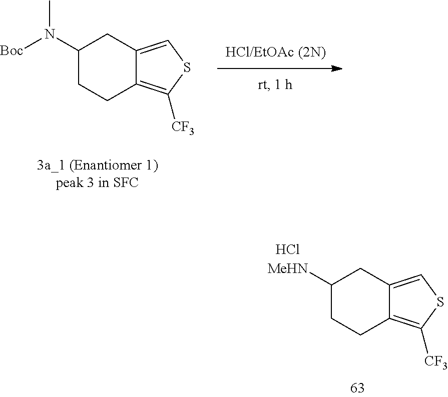 [SMILES: CN(C(=O)OC(C)(C)C)C1CCc2c(csc2C(F)(F)F)C1 ; Cl ; CNC1CCc2c(csc2C(F)(F)F)C1]

To a solution of tert-butyl N-methyl-N-[1-(trifluoromethyl)-4,5,6,7-tetrahydro-2-benzothiophen-5-yl]carbamate (3a_1 enantiomer 1) (100 mg, 297 μmol, 1 eq) in EtOAc (2 mL) was added HCl/EtOAc (4 M, 2 mL). The mixture was stirred at 25° C. for 1 h. The precipitate was collected by filtration, washed with EtOAc and dried in vacuo to give N-methyl-1-(trifluoromethyl)-4,5,6,7-tetrahydro-2-benzothiophen-5-amine hydrochloride salt enantiomer 1 (63) (34.92 mg, 43.24% yield) as a white solid. ¹H NMR (400 MHz, Methanol-d4) δ 7.46 (s, 1H), 3.57-3.50 (m, 1H), 3.42-3.34 (m, 1H), 3.21-3.07 (m, 1H), 2.98-2.76 (m, 5H), 2.41-2.26 (m, 1H), 1.92 (dtd, J=5.7, 11.0, 12.9 Hz, 1H). LCMS: m/z [M+H]⁺=236.0.

### Procedure for the preparation of N-methyl-1-(trifluoromethyl)-4,5,6,7-tetrahydro-2-benzothiophen-5-amine hydrochloride enantiomer 2 (64)

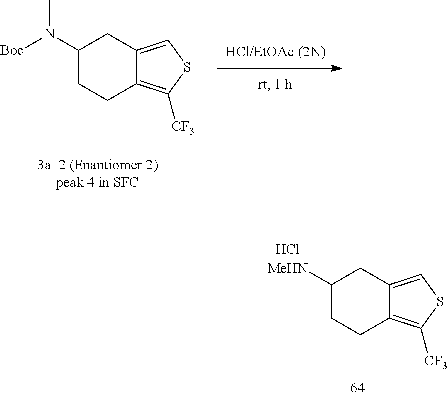 [SMILES: CN(C(=O)OC(C)(C)C)C1CCc2c(csc2C(F)(F)F)C1 ; Cl ; CNC1CCc2c(csc2C(F)(F)F)C1]

To a solution of tert-butyl N-methyl-N-[1-(trifluoromethyl)-4,5,6,7-tetrahydro-2-benzothiophen-5-yl]carbamate (3a_2 enantiomer 2) (100 mg, 298 μmol, 1 eq) in EtOAc (2 mL) was added HCl/EtOAc (4 M, 2 mL). The mixture was stirred at 25° C. for 1 h. The precipitate was collected by filtration, washed with EtOAc and dried in vacuo to give N-methyl-1-(trifluoromethyl)-4,5,6,7-tetrahydro-2-benzothiophen-5-amine hydrochloride salt enantiomer 2 (64) (49.42 mg, 61.00% yield) as a white solid. ¹H NMR (400 MHz, Methanol-d4) δ 7.44 (s, 1H), 3.55-3.43 (m, 1H), 3.36-3.33 (m, 1H), 3.20-3.08 (m, 1H), 2.94-2.85 (m, 1H), 2.83-2.73 (m, 4H), 2.37-2.27 (m, 1H), 1.98-1.83 (m, 1H). LCMS: m/z [M+H]⁺=236.0.

### Synthetic scheme for the preparation of 3-methyl-4,5,6,7-tetrahydrobenzothiophen-6-amine hydrochloride enantiomer 1 (65), and enantiomer 2 (66)

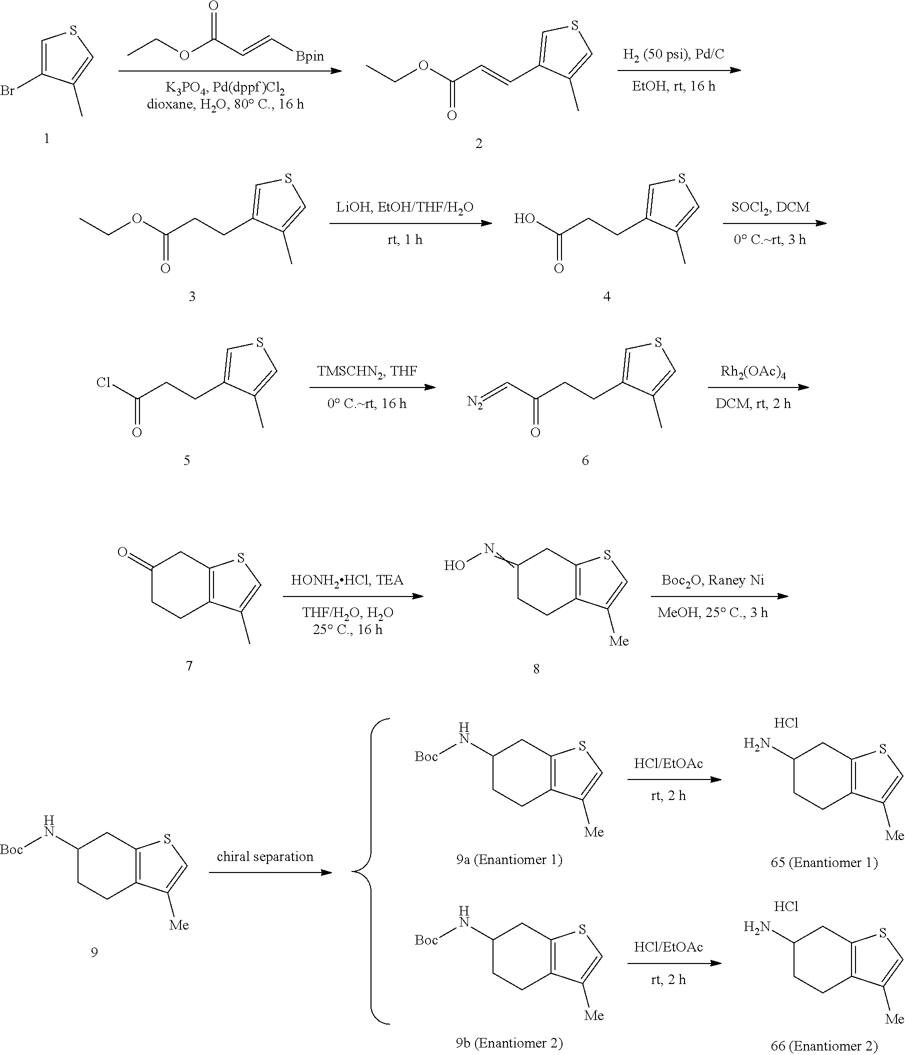

### Procedure for preparation of ethyl (E)-3-(4-methylthiophen-3-yl)acrylate (2)

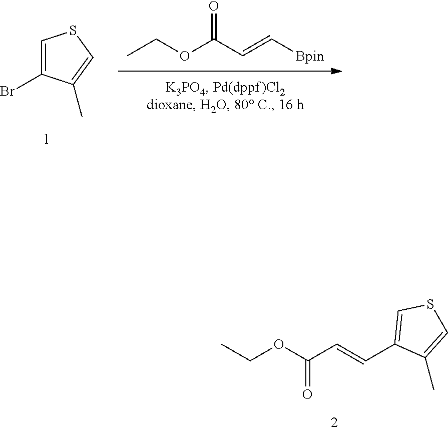

A mixture of 3-bromo-4-methyl-thiophene (1) (12.2 g, 68.90 mmol, 1 eq), ethyl (E)-3-(4,4,5,5-tetramethyl-1,3,2-dioxaborolan-2-yl)prop-2-enoate (24.92 g, 110.24 mmol, 16.41 mL, 1.6 eq) and K₃PO₄ (36.56 g, 172.26 mmol, 2.5 eq) in dioxane (110 mL) and H₂O (11 mL) was added Pd(dppf)Cl₂ (2.52 g, 3.45 mmol, 0.05 eq). The mixture was degassed and purged with N₂ for 3 times, and then the mixture was stirred at 80° C. for 16 hr under N₂ atmosphere. The reaction mixture was extracted with EtOAc 200 mL (100 mL*2). The combined organic layers were washed with NaCl 100 mL (50 mL*2), dried over Na₂SO₄, filtered and concentrated under reduced pressure to give a residue. The residue was purified by flash silica gel chromatography (ISCO®; 80 g SepaFlash® Silica Flash Column, eluent 0~5% Ethyl acetate/Petroleum ether gradient @60 mL/min). Compound ethyl (E)-3-(4-methylthiophen-3-yl)acrylate (2) (12 g, 41.58 mmol, 60.34% yield) was obtained as a yellow solid. ¹H NMR (400 MHz, CHLOROFORM-d) δ=7.66 (d, J=16.1 Hz, 1H), 7.54 (d, J=3.0 Hz, 1H), 6.99-6.94 (m, 1H), 6.29 (d, J=16.0 Hz, 1H), 4.26 (q, J=7.1 Hz, 2H), 2.33 (s, 3H), 1.33 (t, J=7.1 Hz, 3H)

### Procedure for preparation of ethyl 3-(4-methylthiophen-3-yl)propanoate (3)

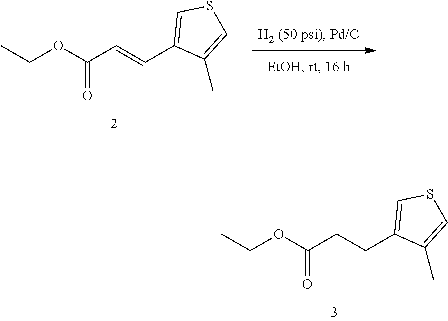

To a solution of ethyl (E)-3-(4-methylthiophen-3-yl)acrylate (2) (12 g, 61.14 mmol, 1 eq) in EtOH (100 mL) was added Pd/C (5 g, 10% Pd) under N₂. The suspension was degassed under vacuum and purged with H₂ several times. The mixture was stirred under H₂ (50 psi) at 60° C. for 16 hours. The reaction mixture was then filtered and the filtrate concentrated under reduced pressure to give a residue. The crude product ethyl 3-(4-methylthiophen-3-yl)propanoate (3) (12 g, crude) was obtained as a white solid and used into the next step without further purification.

### Procedure for preparation of 3-(4-methylthiophen-3-yl)propanoic acid (4)

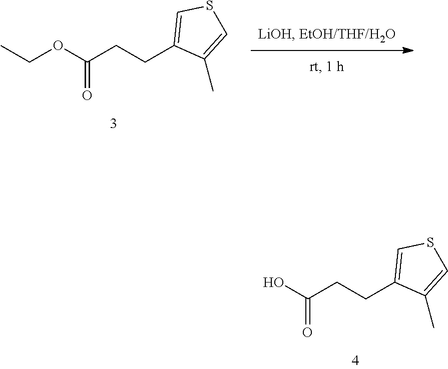

To a solution of ethyl 3-(4-methylthiophen-3-yl)propanoate (3) (12 g, 60.52 mmol, 1 eq) in THF (80 mL), H₂O (80 mL) and EtOH (10 mL) was added LiOH·H₂O (5.08 g, 121.04 mmol, 2 eq). The mixture was stirred at 25° C. for 1 hr. To the mixture was added aq. HCl (2N) to pH 7, and the mixture was then concentrated under reduced pressure to remove THF. Then to the mixture was added aq HCl (1 M) to pH 5-6. The mixture was filtered to yield 3-(4-methylthiophen-3-yl)propanoic acid (4) (6 g, 35.25 mmol, 58.24% yield) as a white solid. LCMS: m/z [M+H]⁺=171.2

### Procedure for preparation of 3-(4-methylthiophen-3-yl)propanoyl chloride (5)

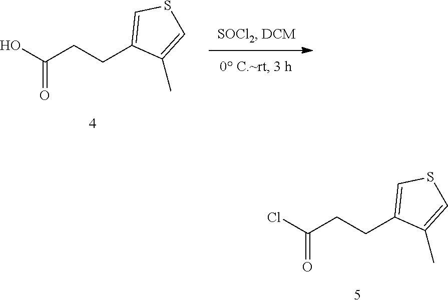

To a solution of 3-(4-methylthiophen-3-yl)propanoic acid (4) (6 g, 35.25 mmol, 1 eq) in DCM (60 mL) was added DMF (51.53 mg, 704.94 μmol, 54.24 μL, 0.02 eq) and then SOCl2 (8.39 g, 70.49 mmol, 5.11 mL, 2 eq) at 0° C. The mixture was then stirred at 20° C. for 3 hr. The reaction mixture was then concentrated under reduced pressure to remove DCM. The crude product 3-(4-methylthiophen-3-yl)propanoyl chloride (5) (6 g, crude) was used into the next step without further purification.

### Procedure for preparation of 1-diazo-4-(4-methylthiophen-3-yl)butan-2-one (6) and 1-diazo-4-(4-methylthiophen-3-yl)-1-(trimethylsilyl)butan-2-one (6A)

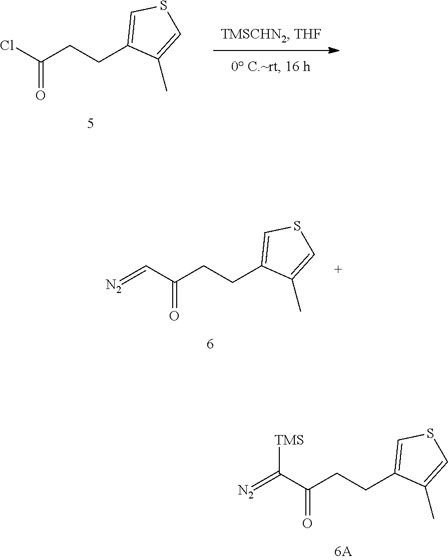

To a solution of 3-(4-methylthiophen-3-yl)propanoyl chloride (5) (6 g, 31.80 mmol, 1 eq) in THF (100 mL) was added TMSCHN₂ (2 M, 47.70 mL, 3 eq). The mixture was stirred at 20° C. for 16 hr. Then the reaction mixture was extracted with EtOAc 50 mL (25 mL*2). The combined organic layers were washed with aq. sat. NaCl (10 mL*2), dried over Na₂SO₄, filtered and concentrated under reduced pressure to give a residue. The residue was purified by flash silica gel chromatography (ISCO®; 80 g SepaFlash® Silica Flash Column, eluent 0~30% Ethyl acetate/Petroleum ether gradient@60 mL/min). Compound 1-diazo-4-(4-methylthiophen-3-yl)butan-2-one (6) (3.4 g, 17.50 mmol, 55.04% yield) was obtained as a yellow liquid and 1-diazo-4-(4-methylthiophen-3-yl)-1-(trimethylsilyl)butan-2-one (6A) (1.1 g, 4.13 mmol, 12.98% yield) was obtained as a yellow liquid. LCMS (6): m/z [M-N₂]⁺=166.1

### Procedure for preparation of 1-diazo-4-(4-methylthiophen-3-yl)butan-2-one (6) from 1-diazo-4-(4-methylthiophen-3-yl)-1-(trimethylsilyl)butan-2-one (6A)

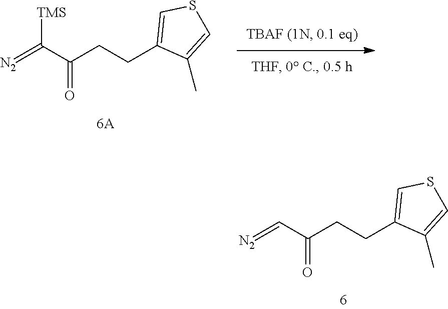

To a solution of 1-diazo-4-(4-methylthiophen-3-yl)-1-(trimethylsilyl)butan-2-one (6A) (1.10 g, 4.13 mmol, 1 eq) in THF (10 mL) was added TBAF (1.08 g, 4.13 mmol, 5 mL, 1 eq). The mixture was stirred at 0° C. for 0.5 hr. The reaction mixture was then concentrated under reduced pressure to remove THF. The residue was purified by flash silica gel chromatography (ISCO®; 40 g SepaFlash® Silica Flash Column, eluent 0~30% Ethyl acetate/Petroleum ether gradient@60 mL/min) to afford 1-diazo-4-(4-methyl-3-thienyl)butan-2-one (6) (600 mg, 3.09 mmol, 74.81% yield).

### Procedure for preparation of 3-methyl-5,7-dihydro-4H-benzothiophen-6-one (7)

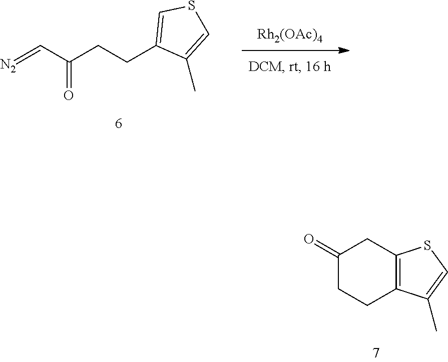

To a solution of 1-diazo-4-(4-methylthiophen-3-yl)butan-2-one (6) (4.00 g, 20.59 mmol, 1 eq) in DCM (1000 mL) was added Rh(OAc)₂ (24.98 mg, 113.05 mol). The mixture was stirred at 25° C. for 16 hr. The reaction mixture was concentrated under reduced pressure to remove DCM. The residue was purified by flash silica gel chromatography (ISCO®; 80 g SepaFlash® Silica Flash Column, eluenteluent 0~40% Ethyl acetate/Petroleum ether gradient=5/1 @60 mL/min) to afford 3-methyl-5,7-dihydro-4H-benzothiophen-6-one (7) (1.3 g, 7.82 mmol, 37.98% yield). LCMS: m/z [M+H]⁺=167.1

### Procedure for preparation of 3-methyl-5,7-dihydro-4H-benzothiophen-6-one oxime (8)

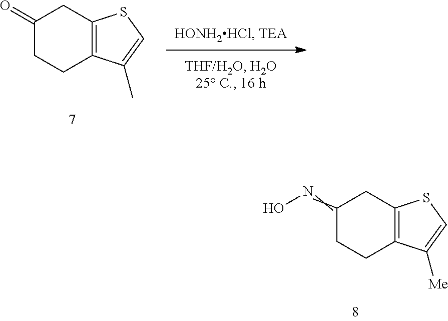

To a solution of 3-methyl-5,7-dihydro-4H-benzothiophen-6-one (7) (1.3 g, 7.82 mmol, 1 eq) in THF (13 mL) and H₂O (13 mL) was added hydroxylamine hydrochloride (1.09 g, 15.64 mmol, 2 eq) and TEA (1.98 g, 19.55 mmol, 2.72 mL, 2.5 eq). The mixture was stirred at 25° C. for 16 hr. The reaction mixture was extracted with EtOAc 10 mL (5 mL*2). The combined organic layers were washed with aq, sat. NaCl 10 mL (5 mL*2), dried over Na₂SO₄, filtered and concentrated under reduced pressure to give a residue. The residue was purified by flash silica gel chromatography (ISCO®; 40 g SepaFlash® Silica Flash Column, eluent 0~20% Ethyl acetate/Petroleum ether gradient @40 mL/min). Compound 3-methyl-5,7-dihydro-4H-benzothiophen-6-one oxime (8) (1 g, 5.52 mmol, 70.55% yield) was obtained as a white solid. LCMS: m/z [M+H]⁺=182.2

### Procedure for preparation of tert-butyl N-(3-methyl-4,5,6,7-tetrahydrobenzothiophen-6-yl)carbamate (9)

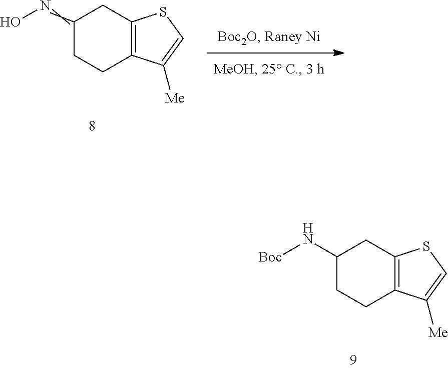 [SMILES: Cc1csc2c1CCC(=NO)C2 ; "Boc2O, Raney Ni" ; Cc1csc2c1CCC(NC(=O)OC(C)(C)C)C2]

To a solution of 3-methyl-5,7-dihydro-4H-benzothiophen-6-one oxime (8) (1 g, 5.52 mmol, 1 eq) and tert-butoxycarbonyl tert-butyl carbonate (1.32 g, 6.07 mmol, 1.39 mL, 1.1 eq) in MeOH (10 mL) was added Raney-Ni (472.68 mg, 5.52 mmol, 1 eq) under N₂ atmosphere. The suspension was degassed and purged with H₂ for 3 times. The mixture was stirred under H₂ (15 psi) at 25° C. for 3 hr. The reaction mixture was then filtered and concentrated under reduced pressure to give a residue. The residue was purified by prep-HPLC (FA condition column: YMC Triart C18 150*25 mm*5 μm; mobile phase: [water(FA)-ACN]; B %: 61%-91%, 8.5 min). Compound tert-butyl N-(3-methyl-4,5,6,7-tetrahydrobenzothiophen-6-yl)carbamate (9) (400 mg, 1.50 mmol, 27.11% yield) was obtained as a white solid.

### Procedure for preparation of tert-butyl N-(3-methyl-4,5,6,7-tetrahydrobenzothiophen-6-yl)carbamate enantiomer 1 (9a) and enantiomer 2 (9b)

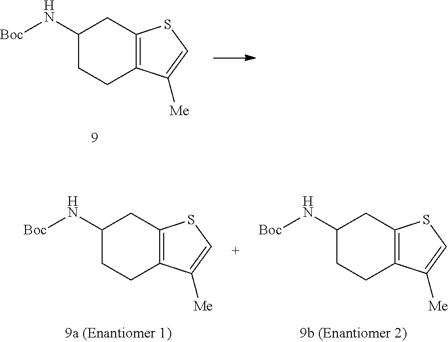 [SMILES: Cc1csc2c1CCC(NC(=O)OC(C)(C)C)C2 ; Cc1csc2c1CCC(NC(=O)OC(C)(C)C)C2 ; Cc1csc2c1CCC(NC(=O)OC(C)(C)C)C2]

Racemic tert-butyl N-(3-methyl-4,5,6,7-tetrahydrobenzothiophen-6-yl)carbamate (9) was chiraly separated (column: DAICEL CHIRALPAK IG (250 mm*30 mm, 10 μm); mobile phase: [0.1% NH₃H₂O MEOH]; B %: 25%-25%, 3.8 min), to give enantiomer 1 (9a, 200 mg, 1^{st} peak in SFC) as a white solid and enantiomer 2 (9b, 250 mg, 2^{nd} peak in SFC) as a white solid.

### Procedure for preparation of 3-methyl-4,5,6,7-tetrahydrobenzothiophen-6-amine hydrochloride enantiomer 1 (65)

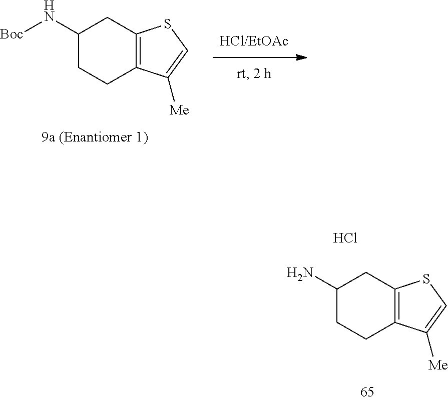 [SMILES: Cc1csc2c1CCC(NC(=O)OC(C)(C)C)C2 ; Cl ; Cc1csc2c1CCC(N)C2]

To a solution of tert-butyl N-(3-methyl-4,5,6,7-tetrahydrobenzothiophen-6-yl)carbamate (9a) (150 mg, 560.99 μmol, 1 eq) in EtOAc (1 mL) was added HCl/EtOAc (4 M, 2 mL, 14.26 eq). The mixture was stirred at 25° C. for 2 hr. The reaction mixture was concentrated under reduced pressure to remove EtOAc. Compound 3-methyl-4,5,6,7-tetrahydrobenzothiophen-6-amine hydrochloride enantiomer 1 (65) (92.25 mg, 452.80 mol 80.72% yield) was obtained as a white solid. ¹H NMR (400 MHz, METHANOL-d₄) δ=6.84 (s, 1H), 6.76-6.71 (m, 1H), 3.65-3.56 (m, 1H), 3.19 (br dd, J=4.8, 16.1 Hz, 1H), 2.89-2.78 (m, 1H), 2.75-2.57 (m, 2H), 2.27-2.16 (m, 1H), 2.10 (s, 3H), 1.93 (dt, J=3.6, 6.3 Hz, 1H). LCMS: m/z [M+H]⁺=168.08

### Procedure for preparation of 3-methyl-4,5,6,7-tetrahydrobenzothiophen-6-amine hydrochloride enantiomer 2 (66)

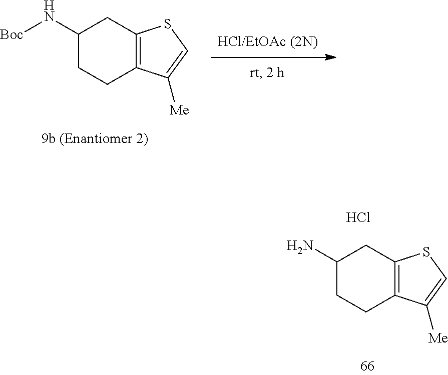 [SMILES: Cc1csc2c1CCC(NC(=O)OC(C)(C)C)C2 ; "HCl/EtOAc (2N)" ; Cl ; Cc1csc2c1CCC(N)C2]

To a solution of tert-butyl N-(3-methyl-4,5,6,7-tetrahydrobenzothiophen-6-yl)carbamate (9b) (220 mg, 822.78 μmol, 1 eq) in EtOAc (1 mL) was added HCl/EtOAc (4 M, 2.93 mL, 14.26 eq). The mixture was stirred at 25° C. for 2 hr. The reaction mixture was concentrated under reduced pressure to remove EtOAc. Compound 3-methyl-4,5,6,7-tetrahydrobenzothiophen-6-amine hydrochloride enantiomer 2 (66) (132.75 mg, 651.59 mol, 79.19% yield) was obtained as a white solid. ¹H NMR (400 MHz, METHANOL-d₄) δ=6.84 (s, 1H), 3.66-3.55 (m, 1H), 3.19 (br dd, J=4.2, 15.9 Hz, 1H), 2.92-2.78 (m, 1H), 2.74-2.59 (m, 2H), 2.22 (br dd, J=2.6, 8.9 Hz, 1H), 2.10 (s, 3H), 1.98-1.87 (m, 1H). LCMS: m/z [M+H]⁺=168.08

### Synthetic scheme for preparation of 2,3-dimethyl-4,5,6,7-tetrahydrobenzo[b]thiophen-6-amine hydrochloride enantiomer 1 (67) and enantiomer 2 (68)

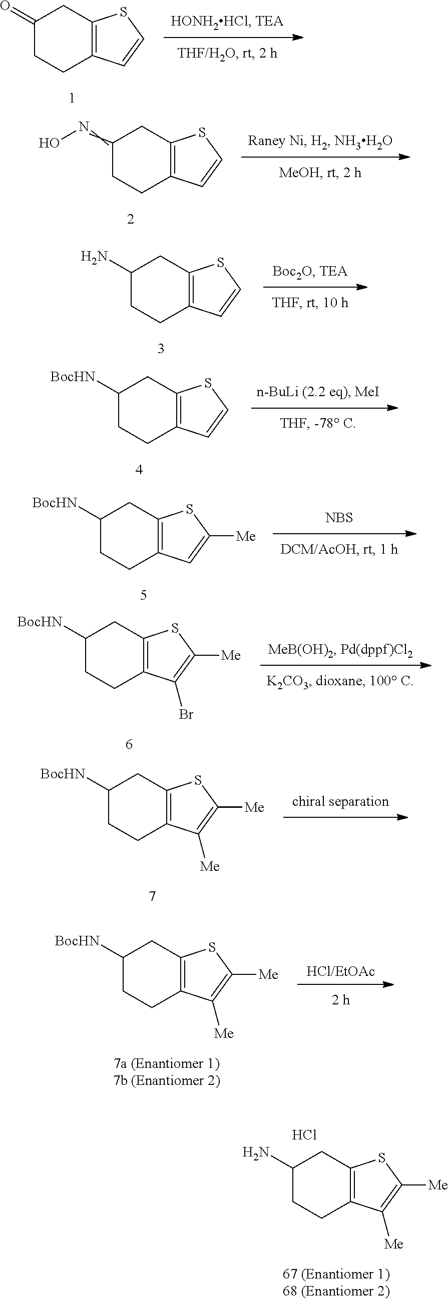

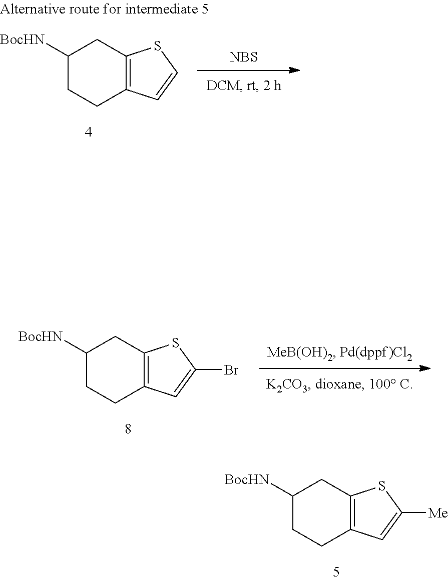

### Procedure for preparation of 5,7-dihydro-4H-benzothiophen-6-one oxime (2)

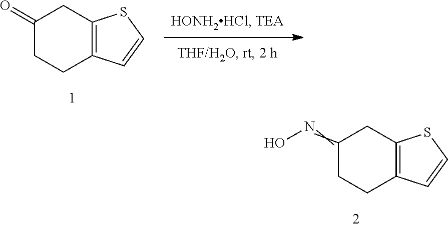 [SMILES: O=C1CCc2ccsc2C1 ; ON=C1CCc2ccsc2C1]

To a solution of 5, 7-dihydro-4H-benzothiophen-6-one (1) (4 g, 26.3 mmol, 1 eq) in THF (20 mL) was added NH₂OH·HCl (3.65 g, 52.6 mmol, 2 eq) and TEA (7.98 g, 78.8 mmol, 10.9 mL, 3 eq). The mixture was stirred at 25° C. for 2 hr. The reaction mixture was extracted with EtOAc (20 mL*2). The combined organic layers were washed with aq. sat. NaCl (20 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~30% Ethyl acetate/Petroleum ether gradient @40 mL/min) to yield 5, 7-dihydro-4H-benzothiophen-6-one oxime (2) (4.2 g, 25.12 mmol, 95.57% yield) as a yellow solid. ¹H NMR (400 MHz, CHLOROFORM-d) δ=7.21-7.07 (m, 1H), 6.80 (d, J=5.1 Hz, 1H), 3.90 (s, 2H), 2.91-2.79 (m, 2H), 2.68-2.57 (m, 2H)

### Procedure for preparation of 4,5,6,7-tetrahydrobenzothiophen-6-amine (3)

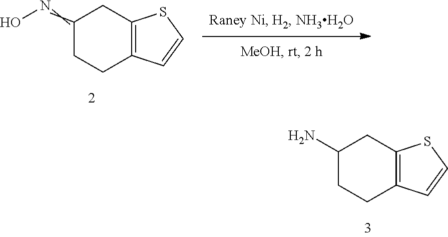 [SMILES: ON=C1CCc2ccsc2C1 ; NC1CCc2ccsc2C1]

To a solution of 5, 7-dihydro-4H-benzothiophen-6-one oxime (2) (4.1 g, 24.5 mmol, 1 eq) in MeOH (40 mL) was added Raney-Ni (2.10 g, 24.5 mmol, 1 eq) and NH₃H₂O (2.46 g, 49.0 mmol, 2.70 mL, 2 eq) under N₂ atmosphere. The suspension was degassed and purged with H₂ for 3 times. The mixture was stirred under H₂ (15 Psi) at 25° C. for 2 hr. The reaction solution was diluted with MeOH (10 mL) and the supernatant was collected and concentrated to yield 4, 5, 6, 7-tetrahydrobenzothiophen-6-amine (3) (3 g, crude) as a yellow oil. LCMS: m/z [M-NH₂]⁺=137.0

### Procedure for preparation of tert-butyl N-(4,5,6,7-tetrahydrobenzothiophen-6-yl)carbamate (4)

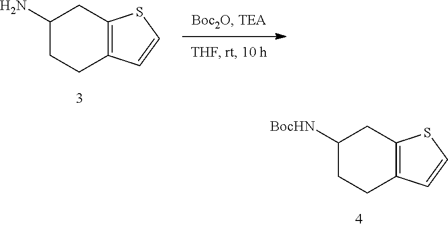 [SMILES: NC1CCc2ccsc2C1 ; CC(C)(C)OC(=O)NC1CCc2ccsc2C1]

To a solution of 4, 5, 6, 7-tetrahydrobenzothiophen-6-amine (3) (3 g, 19.6 mmol, 1 eq) in THF (30 mL) was added TEA (3.96 g, 39.2 mmol, 5.45 mL, 2 eq) and (Boc)₂O (8.55 g, 39.2 mmol, 8.99 mL, 2 eq). The mixture was stirred at 25° C. for 10 hr. The reaction mixture was concentrated under reduced pressure to give a residue. The residue was purified by flash silica gel chromatography (ISCO®; 12 g SepaFlash® Silica Flash Column, eluent 0~5% Ethyl acetate/Petroleum ether gradient @40 mL/min) to yield tert-butyl N-(4, 5, 6, 7-tetrahydrobenzothiophen-6-yl) carbamate (4) (2.4 g, 9.47 mmol, 48.39% yield) as a yellow oil. ¹H NMR (400 MHz, CHLOROFORM-d) δ=7.02 (d, J=5.0 Hz, 1H), 6.69 (d, J=5.1 Hz, 1H), 4.65-4.52 (m, 1H), 4.11-3.91 (m, 1H), 3.07 (br dd, J=4.8, 16.1 Hz, 1H), 2.73-2.51 (m, 3H), 2.02-1.85 (m, 1H), 1.75 (td, J=6.9, 13.9 Hz, 1H), 1.38 (s, 9H)

### Procedure for preparation of tert-butyl N-(2-methyl-4,5,6,7-tetrahydrobenzothiophen-6-yl)carbamate (5)

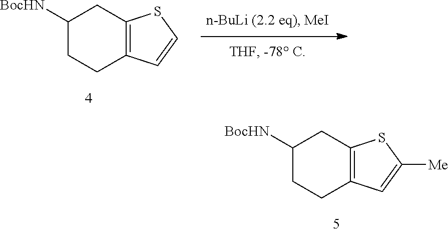 [SMILES: CC(C)(C)OC(=O)NC1CCc2ccsc2C1 ; Cc1cc2c(s1)CC(NC(=O)OC(C)(C)C)CC2]

To a solution of tert-butyl N-(4, 5, 6, 7-tetrahydrobenzothiophen-6-yl) carbamate (4) (2.3 g, 9.08 mmol, 1 eq) in THF (25 mL) was added dropwise n-BuLi (2.5 M, 7.99 mL, 2.2 eq) at −78° C. After addition, the mixture was stirred at this temperature for 0.5 hr, and then MeI (1.55 g, 10.9 mmol, 678 μL, 1.2 eq) was added dropwise at −78° C. The reaction mixture was poured into aq. NH₄Cl 10 mL and extracted with EtOAc (20 mL*2). The combined organic layers were washed with aq. sat. NaCl (20 mL), dried over anhydrous Na₂SO₄, filtered and concentrated under reduced pressure to give a residue. The residue was purified by flash silica gel chromatography (ISCO®; 4 g SepaFlash® Silica Flash Column, eluent 0~10% Ethyl acetate/Petroleum ether gradient @40 mL/min). The residue was purified by prep-HPLC (column: Waters Xbridge 150*25 mm*5 μm; mobile phase: [water (NH₄HCO₃)-ACN]; B %: 53%-83%, 8 min) to yield tert-butyl N-(2-methyl-4, 5, 6, 7-tetrahydrobenzothiophen-6-yl) carbamate (5) (140 mg, 523.59 μmol, 5.77% yield) as a yellow oil. ¹H NMR (400 MHz, CHLOROFORM-d) δ=6.41 (s, 1H), 4.66 (br d, J=2.6 Hz, 1H), 4.06 (br d, J=2.4 Hz, 1H), 3.06 (br dd, J=4.8, 16.0 Hz, 1H), 2.82 (s, 1H), 2.67-2.53 (m, 3H), 2.41 (s, 3H), 2.01-1.89 (m, 1H), 1.84-1.72 (m, 1H), 1.46 (s, 9H)

### Procedure for preparation of tert-butyl N-(2-bromo-4,5,6,7-tetrahydrobenzothiophen-6-yl)carbamate (8)

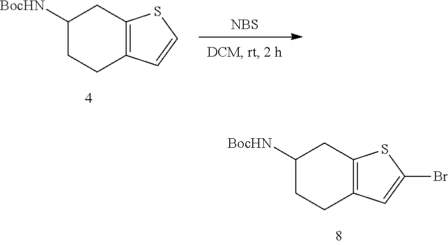

To a solution of tert-butyl N-(4, 5, 6, 7-tetrahydrobenzothiophen-6-yl) carbamate (4) (240 mg, 947 μmol, 1 eq) in DCM (5 mL) was added NBS (185 mg, 1.04 mmol, 1.1 eq). The mixture was stirred at 25° C. for 2 hr. The reaction mixture was extracted with EtOAc (10 mL*3). The combined organic layers were washed with aq. sat. NaCl (20 mL), dried over anhydrous Na₂SO₄, filtered and concentrated under reduced pressure to give a residue. The residue was purified by flash silica gel chromatography (ISCO®; 4 g SepaFlash® Silica Flash Column, eluent 0~17% Petroleum ether gradient/Ethyl acetate @40 mL/min) to afford tert-butyl N-(2-bromo-4, 5, 6, 7-tetrahydrobenzothiophen-6-yl) carbamate (8) (260 mg, 782.53 μmol, 82.61% yield) as a yellow oil. ¹H NMR (400 MHz, CHLOROFORM-d) δ=6.73 (s, 1H), 4.68-4.61 (m, 1H), 4.10-4.00 (m, 1H), 3.06 (br dd, J=4.7, 16.1 Hz, 1H), 2.68 (br t, J=6.3 Hz, 2H), 2.55 (br dd, J=7.1, 16.3 Hz, 1H), 2.05-1.94 (m, 1H), 1.86-1.73 (m, 1H), 1.47 (s, 9H)

### Procedure for preparation of tert-butyl N-(2-methyl-4,5,6,7-tetrahydrobenzothiophen-6-yl)carbamate (5)

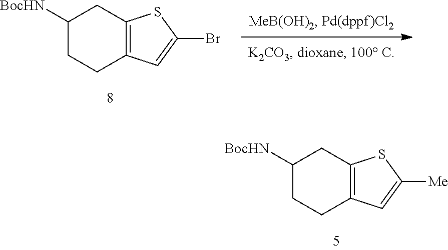 [SMILES: CC(C)(C)OC(=O)NC1CCc2cc(Br)sc2C1 ; "MeB(OH)2, Pd(dppf)Cl2" ; Cc1cc2c(s1)CC(NC(=O)OC(C)(C)C)CC2]

A mixture of tert-butyl N-(2-bromo-4, 5, 6, 7-tetrahydrobenzothiophen-6-yl) carbamate (8) (260 mg, 782 μmol, 1 eq), Pd(dppf)Cl₂ (57.3 mg, 78.3 μmol, 0.1 eq), MeB(OH)₂ (140 mg, 2.35 mmol, 3 eq) and K₂CO₃ (216 mg, 1.57 mmol, 2 eq) in dioxane (5 mL) and H₂O (1 mL) was degassed and purged with N₂ for 3 times, and then the mixture was stirred at 100° C. for 2 hr under N₂ atmosphere. The reaction mixture was extracted with EtOAc (20 mL*3). The combined organic layers were washed with aq. sat. NaCl (20 mL), dried over anhydrous Na₂SO₄, filtered and concentrated under reduced pressure to give a residue. The residue was purified by flash silica gel chromatography (ISCO®; 4 g SepaFlash® Silica Flash Column, eluent 0~20% Petroleum ether gradient/Ethyl acetate @40 mL/min) to yield tert-butyl N-(2-methyl-4, 5, 6, 7-tetrahydrobenzothiophen-6-yl) carbamate (5) (120 mg, 448 mol) as a yellow solid. ¹H NMR (400 MHz, CHLOROFORM-d) 6=6.34 (s, 1H), 4.59 (br d, J=5.0 Hz, 1H), 4.03-3.91 (m, 1H), 2.98 (br dd, J=4.8, 15.8 Hz, 1H), 2.60-2.45 (m, 3H), 2.33 (s, 3H), 1.88 (dtd, J=2.8, 6.2, 12.6 Hz, 1H), 1.78-1.65 (m, 1H), 1.43-1.33 (m, 9H)

### Procedure for preparation of tert-butyl N-(3-bromo-2-methyl-4,5,6,7-tetrahydrobenzothiophen-6-yl)carbamate (6)

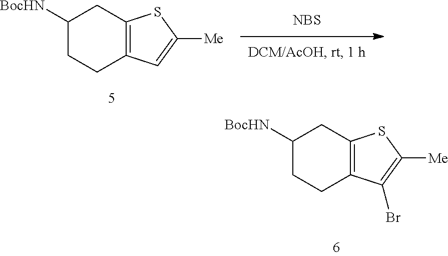 [SMILES: Cc1cc2c(s1)CC(NC(=O)OC(C)(C)C)CC2 ; "NBS" ; Cc1sc2c(c1Br)CCC(NC(=O)OC(C)(C)C)C2]

To a solution of tert-butyl N-(2-methyl-4, 5, 6, 7-tetrahydrobenzothiophen-6-yl) carbamate (5) (560 mg, 2.09 mmol, 1 eq) in DCM (5 mL) and AcOH (1 mL) was added NBS (410 mg, 2.30 mmol, 1.1 eq). The mixture was stirred at 25° C. for 1 hr. The reaction mixture was extracted with EtOAc (10 mL*3). The combined organic layers were washed with sat. NaCl (20 mL), dried over anhydrous Na₂SO₄, filtered and concentrated under reduced pressure to give a residue. The residue was purified by flash silica gel chromatography (ISCO®; 4 g SepaFlash® Silica Flash Column, eluent 0~17% Petroleum ether/Ethyl acetate gradient @40 mL/min) to yield tert-butyl N-(3-bromo-2-methyl-4, 5, 6, 7-tetrahydrobenzothiophen-6-yl) carbamate (6) (370 mg, 1.07 mmol, 51.02% yield) as a yellow solid. ¹H NMR (400 MHz, CHLOROFORM-d) δ=4.62-4.50 (m, 1H), 4.02-3.93 (m, 1H), 3.05-2.91 (m, 1H), 2.58-2.44 (m, 3H), 2.28 (s, 3H), 1.91 (dtd, J=2.6, 6.3, 12.6 Hz, 1H), 1.83-1.70 (m, 1H), 1.38 (s, 9H)

### Procedure for preparation of tert-butyl N-(2,3-dimethyl-4,5,6,7-tetrahydrobenzothiophen-6-yl)carbamate enantiomer 1 (7a) and enantiomer 2 (7b)

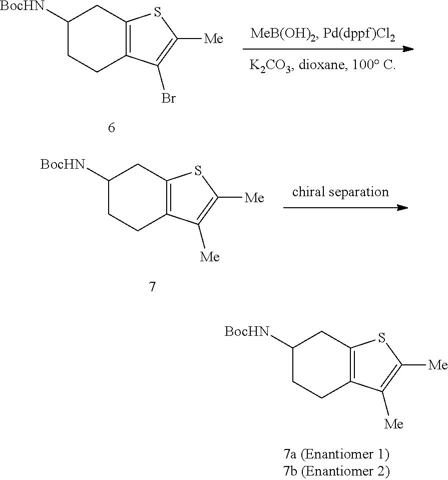 [SMILES: Cc1sc2c(c1Br)CCC(NC(=O)OC(C)(C)C)C2 ; "MeB(OH)2, Pd(dppf)Cl2" ; Cc1sc2c(c1C)CCC(NC(=O)OC(C)(C)C)C2 ; "chiral separation" ; Cc1sc2c(c1C)CCC(NC(=O)OC(C)(C)C)C2]

A mixture of tert-butyl N-(3-bromo-2-methyl-4,5,6,7-tetrahydrobenzothiophen-6-yl)carbamate (6) (370 mg, 1.07 mmol, 1 eq), Pd(dppf)Cl₂ (78.2 mg, 107 μmol, 0.1 eq), MeB(OH)₂ (192 mg, 3.21 mmol, 3 eq) and K₂CO₃ (295 mg, 2.14 mmol, 2 eq) in dioxane (5 mL) and H₂O (1 mL) was degassed and purged with N₂ for 3 times, and then the mixture was stirred at 100° C. for 16 hr under N₂ atmosphere. The reaction mixture was extracted with EtOAc (10 mL*3). The combined organic layers were washed with aq. sat. NaCl (10 mL), dried over anhydrous Na₂SO₄, filtered and concentrated under reduced pressure to give a residue. The residue was purified by flash silica gel chromatography (ISCO®; 4 g SepaFlash® Silica Flash Column, eluent 0~20% Petroleum ether/Ethyl acetate gradient @40 mL/min) to yield racemic tert-butyl N-(2,3-dimethyl-4,5,6,7-tetrahydrobenzothiophen-6-yl)carbamate (7), (230 mg, 817.30 mol) as a yellow solid. LCMS: m/z [M+H-Boc]=226.1.

The racemic product was subjected to prep-Chiral-SFC separation (column: DAICEL CHIRALPAK AD (250 mm*30 mm, 10 μm); mobile phase: [0.1% NH₃H₂O MEOH]; B %: 20%-20%, 3.5 min) to afford the 1^{st} enantiomer (7a) (RT=0.780 min, 85 mg) and the 2^{nd} enantiomer (7b) (RT=0.872 min, 90 mg), both as white solids.

### Procedure for preparation of 2, 3-dimethyl-4, 5, 6, 7-tetrahydrobenzothiophen-6-amine hydrochloride enantiomer 1 (67)

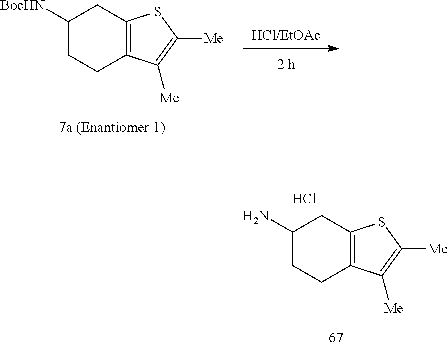 [SMILES: Cc1sc2c(c1C)CCC(NC(=O)OC(C)(C)C)C2 ; "HCl/EtOAc" ; Cl ; Cc1sc2c(c1C)CCC(N)C2]

To a solution of tert-butyl N-(2,3-dimethyl-4,5,6,7-tetrahydrobenzothiophen-6-yl)carbamate enantiomer 1 (7a) (85 mg, 302 μmol, 1 eq) in EtOAc (2 mL) was added HCl/EtOAc (4 M, 5 mL, 66.2 eq). The mixture was stirred at 25° C. for 2 hr. The reaction mixture was filtered and the cake was washed with EtOAc (10 mL) to afford 2, 3-dimethyl-4, 5, 6, 7-tetrahydrobenzothiophen-6-amine hydrochloride enantiomer 1 (67) (33.32 mg, 150.11 μmol, 49.70% yield) was obtained as an off-white solid. ¹H NMR (400 MHz, METHANOL-d₄) δ=3.64-3.54 (m, 1H), 3.10 (br dd, J=5.3, 15.7 Hz, 1H), 2.75 (br dd, J=8.3, 16.3 Hz, 1H), 2.68-2.53 (m, 2H), 2.28 (s, 3H), 2.23-2.15 (m, 1H), 1.97 (s, 3H), 1.94-1.84 (m, 1H). LCMS: m/z [M-NH2]=165.1

### Procedure for preparation of 2, 3-dimethyl-4, 5, 6, 7-tetrahydrobenzothiophen-6-amine hydrochloride enantiomer 2 (68)

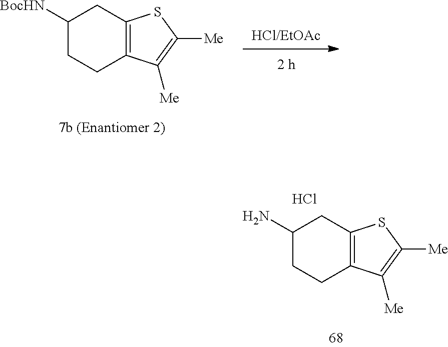 [SMILES: Cc1sc2c(c1C)CCC(NC(=O)OC(C)(C)C)C2 ; "HCl/EtOAc" ; Cl ; Cc1sc2c(c1C)CCC(N)C2]

To a solution of tert-butyl N-(2,3-dimethyl-4,5,6,7-tetrahydrobenzothiophen-6-yl)carbamate enantiomer 2 (7b) (90 mg, 319 μmol, 1 eq) in EtOAc (2 mL) was added HCl/EtOAc (4 M, 5 mL, 62.54 eq). The mixture was stirred at 25° C. for 2 hr. The reaction mixture was filtered and the cake was washed with EtOAc (10 mL) to afford 2,3-dimethyl-4,5,6,7-tetrahydrobenzothiophen-6-amine hydrochloride enantiomer 2 (68) (23.32 mg, 105.06 μmol, 32.85% yield) was obtained as an off-white solid. ¹H NMR (400 MHz, METHANOL-d4) δ=3.64-3.53 (m, 1H), 3.10 (br dd, J=5.1, 15.8 Hz, 1H), 2.75 (br dd, J=8.8, 16.0 Hz, 1H), 2.70-2.51 (m, 2H), 2.28 (s, 3H), 2.24-2.15 (m, 1H), 1.97 (s, 3H), 1.95-1.84 (m, 1H). LCMS: m/z [M-NH₂]=165.1

### Synthetic scheme for preparation of N, 3-dimethyl-4,5,6,7-tetrahydrobenzo[b]thiophen-5-amine hydrochloride enantiomer 1 (69), and enantiomer 2 (70)

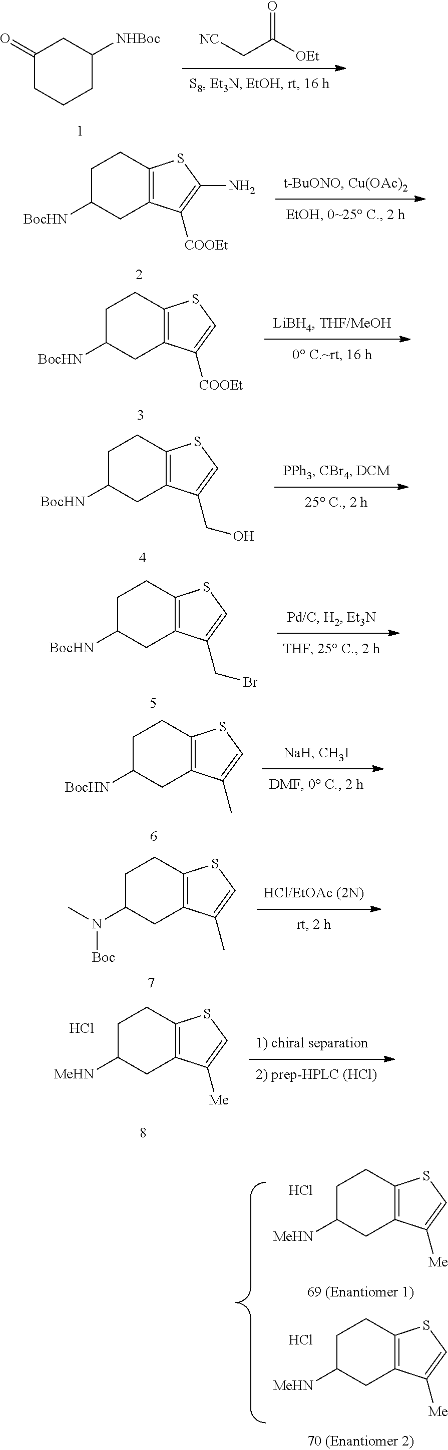

### Procedure for preparation of ethyl 2-amino-5-(tert-butoxycarbonylamino)-4,5,6,7-tetrahydrobenzothiophene-3-carboxylate (2)

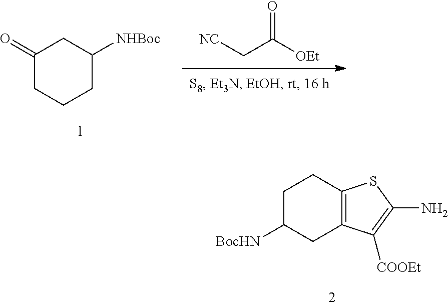 [SMILES: CC(C)(C)OC(=O)NC1CCCC(=O)C1 ; CCOC(=O)CC#N ; CCOC(=O)c1c(N)sc2c1CC(NC(=O)OC(C)(C)C)CC2]

To a mixture of tert-butyl N-(3-oxocyclohexyl) carbamate (1) (10 g, 46.89 mmol, 1 eq), S₈ (2.41 g, 75.16 mmol, 1.60 eq) and ethyl 2-cyanoacetate (6.36 g, 56.27 mmol, 6.00 mL, 1.2 eq) in EtOH (100 mL) was added TEA (9.49 g, 93.78 mmol, 13.05 mL, 2 eq) in one portion at 10° C. under N₂. The mixture was stirred at 25° C. for 16 hours. The mixture was quenched with H₂O (100 mL). The mixture was extracted with EtOAc (300 mL*3). The combined organic phase was washed with brine (200 mL*3), dried over Na₂SO₄, filtered and concentrated at reduced pressure to give a residue. The residue was purified by silica gel chromatography (100-200 mesh silica gel, Petroleum ether/Ethyl acetate=1/0~3/1, Rf=0.42) to afford ethyl 2-amino-5-(tert-butoxycarbonylamino)-4,5,6,7-tetrahydrobenzothiophene-3-carboxylate (2) (13 g, 38.19 mmol, 81.44% yield) as a white solid. ¹H NMR (400 MHz, CDCl3, 296 K) δ 6.10 (s, 2H), 4.24-4.18 (m, 2H), 3.90 (br d, J=7.0 Hz, 1H), 3.04 (br dd, J=5.1, 17.1 Hz, 1H), 2.59-2.52 (m, 2H), 2.48 (br d, J=7.1 Hz, 1H), 1.96 (ddd, J=3.0, 6.1, 9.0 Hz, 1H), 1.74 (br dd, J=7.6, 12.6 Hz, 1H), 1.42 (s, 9H), 1.29 (t, J=7.1 Hz, 3H). LCMS: m/z [M+H]⁺=341.1.

### Procedure for preparation of ethyl 5-(tert-butoxycarbonylamino)-4,5,6,7-tetrahydrobenzothiophene-3-carboxylate (3)

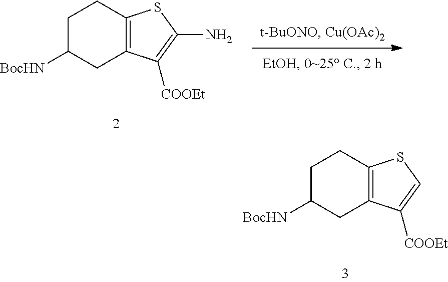

To a mixture of ethyl 2-amino-5-(tert-butoxycarbonylamino)-4,5,6,7-tetrahydrobenzothiophene-3-carboxylate (2) (13 g, 38.19 mmol, 1 eq) and Cu(OAc)₂ (13.87 g, 76.37 mmol, 2 eq) in EtOH (150 mL) was added tert-butyl nitrite (3.94 g, 38.19 mmol, 4.54 mL, 1 eq) at 0° C. under N₂. The mixture was stirred at 25° C. for 2 hours. The mixture was extracted with EtOAc (300 mL*3). The combined organic phase was washed with brine (200 mL*3), dried over Na₂SO₄, filtered and concentrated at reduced pressure to give a residue. The residue was purified by silica gel chromatography (100-200 mesh silica gel, Petroleum ether/Ethyl acetate=1/0~5/1, Rf=0.79) to afford ethyl 5-(tert-butoxycarbonylamino)-4,5,6,7-tetrahydrobenzothiophene-3-carboxylate (3) (5.4 g, 16.59 mmol, 43.45% yield) as a white solid. LCMS: m/z [M+H]⁺=226.2. ¹H NMR (400 MHz, CDCl3, 298 K) δ (ppm)=7.94 (s, 1H), 4.32 (s, 2H), 4.00 (br s, 1H), 3.28 (dd, J=4.8, 17.1 Hz, 1H), 2.86 (br t, J=6.4 Hz, 2H), 2.71 (dd, J=7.6, 17.1 Hz, 1H), 2.15-2.06 (m, 1H), 1.90-1.78 (m, 1H), 1.49-1.45 (m, 9H), 1.35 (t, J=7.1 Hz, 3H)

### Procedure for preparation of tert-butyl N-[3-(hydroxymethyl)-4,5,6,7-tetrahydrobenzothiophen-5-yl]carbamate (4)

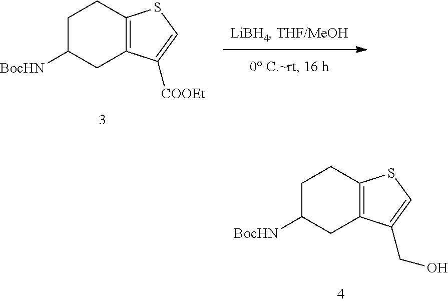 [SMILES: CCOC(=O)c1csc2c1CC(NC(=O)OC(C)(C)C)CC2 ; CC(C)(C)OC(=O)NC1CCc2scc(CO)c2C1]

To a mixture of ethyl 5-(tert-butoxycarbonylamino)-4,5,6,7-tetrahydrobenzothiophene-3-carboxylate (3) (2 g, 6.15 mmol, 1 eq) in THF (20 mL) was added LiBH₄ (535.52 mg, 24.58 mmol, 4 eq) and MeOH (2 mL) in one portion at 0° C. under N₂. The mixture was stirred at 25° C. for 16 hours. The mixture was quenched with saturated aq. ammonium chloride solution (20 mL). The mixture was extracted with DCM (50 mL*3). The combined organic phase was washed with brine (20 mL*3), dried over Na₂SO₄, filtered and concentrated at reduced pressure to give a residue. The residue was purified by silica gel chromatography (100-200 mesh silica gel, Petroleum ether/Ethyl acetate=1/0~1/1, Rf=0.13) to afford tert-butyl N-[3-(hydroxymethyl)-4,5,6,7-tetrahydrobenzothiophen-5-yl]carbamate (4) (1.45 g, 5.12 mmol, 83.25% yield) as a white solid.

### Alternative procedure for preparation of tert-butyl N-[3-(hydroxymethyl)-4,5,6,7-tetrahydrobenzothiophen-5-yl]carbamate (4)

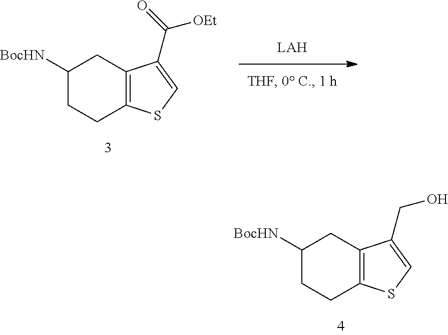 [SMILES: CCOC(=O)c1csc2c1CC(NC(=O)OC(C)(C)C)CC2 ; CC(C)(C)OC(=O)NC1CCc2scc(CO)c2C1]

To a solution of ethyl 5-(tert-butoxycarbonylamino)-4,5,6,7-tetrahydrobenzothiophene-3-carboxylate (3) (3 g, 9.22 mmol, 1 eq) in THF (20 mL) was added LiAlH₄ (700 mg, 18.4 mmol, 2 eq) at 0° C. The mixture was stirred at 0° C. for 1 hr. The reaction mixture was diluted with water (30 mL) and concentrated under vacuum to remove THF. The aqueous phase was extracted with EtOAc (30 mL*3). The combined organic phase was washed with brine (30 mL), dried over anhydrous sodium sulfate, filtered and concentrated under vacuum to give a residue. The residue was purified by flash silica gel chromatography (ISCO®; 12 g SepaFlash® Silica Flash Column, eluent 0~65% Ethyl acetate/Petroleum ether gradient @40 mL/min) to yield tert-butyl N-[3-(hydroxymethyl)-4,5,6,7-tetrahydrobenzothiophen-5-yl]carbamate (4) (2.1 g, 7.41 mmol) as a white solid. ¹H NMR (400 MHz, CHLOROFORM-d) δ 7.03 (s, 1H), 4.56-4.52 (m, 2H), 4.01 (br s, 1H), 3.05-2.90 (m, 1H), 2.86 (br t, J=5.9 Hz, 2H), 2.43 (br dd, J=7.5, 16.0 Hz, 1H), 2.12-2.02 (m, 1H), 1.90-1.79 (m, 1H), 1.46 (s, 9H). LCMS: m/z [M-OH]⁺=266.3

### Procedure for preparation of tert-butyl N-[3-(bromomethyl)-4,5,6,7-tetrahydrobenzothiophen-5-yl]carbamate (5)

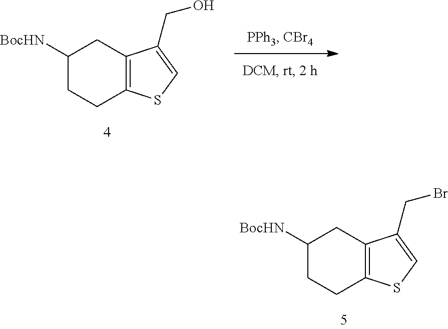 [SMILES: CC(C)(C)OC(=O)NC1CCc2scc(CO)c2C1 ; CC(C)(C)OC(=O)NC1CCc2scc(CBr)c2C1]

To a solution of tert-butyl N-[3-(hydroxymethyl)-4,5,6,7-tetrahydrobenzothiophen-5-yl]carbamate (4) (1.7 g, 6.00 mmol, 1 eq) in DCM (20 mL) was added CBr₄ (2.39 g, 7.20 mmol, 1.2 eq) and PPh₃ (2.05 g, 7.80 mmol, 1.3 eq) at 0° C. The mixture was stirred at 20° C. for 2 hrs. The reaction mixture was concentrated under vacuum to give a residue. The residue was purified by flash silica gel chromatography (ISCO®; 4 g SepaFlash® Silica Flash Column, eluent 0~6% Ethyl acetate/Petroleum ether gradient @40 mL/min) to afford tert-butyl N-[3-(bromomethyl)-4,5,6,7-tetrahydrobenzothiophen-5-yl]carbamate (5) (1.4 g, 4.04 mmol) as a white solid. ¹H NMR (400 MHz, CHLOROFORM-d) δ 7.13 (s, 1H), 4.72-4.56 (m, 1H), 4.40 (s, 2H), 4.13-3.99 (m, 1H), 2.99 (br dd, J=4.6, 15.4 Hz, 1H), 2.91-2.81 (m, 2H), 2.49 (br dd, J=7.7, 15.8 Hz, 1H), 2.17-2.03 (m, 1H), 1.93-1.78 (m, 1H), 1.47 (s, 9H)

### Procedure for preparation of tert-butyl N-(3-methyl-4,5,6,7-tetrahydrobenzothiophen-5-yl)carbamate (6)

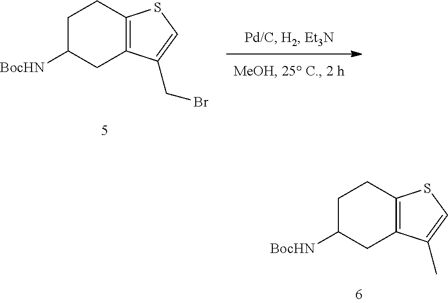 [SMILES: CC(C)(C)OC(=O)NC1CCc2scc(CBr)c2C1 ; Cc1csc2c1CC(NC(=O)OC(C)(C)C)CC2]

To a solution of tert-butyl N-[3-(bromomethyl)-4,5,6,7-tetrahydrobenzothiophen-5-yl]carbamate (5) (700 mg, 2.02 mmol, 1 eq) and TEA (409.11 mg, 4.04 mmol, 562.73 μL, 2 eq) in MeOH (10 mL) was added Pd/C (200 mg, 10% Pd) under N₂. The suspension was degassed under vacuum and purged with H₂ several times. The mixture was stirred under H₂ (15 psi, 1.5 L) at 25° C. for 2 hours. The reaction mixture was filtered and the filtrate was concentrated. The residue was purified by silica gel chromatography (100-200 mesh silica gel, Petroleum ether/Ethyl acetate=1/0~5/1, Rf=0.64) to afford tert-butyl N-(3-methyl-4,5,6,7-tetrahydrobenzothiophen-5-yl)carbamate (6) (460 mg, 1.72 mmol, 85.10% yield) as a white solid. LCMS: m/z [M+H]⁺=212.1.

### Alternative procedure for preparation of tert-butyl N-(3-methyl-4,5,6,7-tetrahydrobenzothiophen-5-yl)carbamate (6)

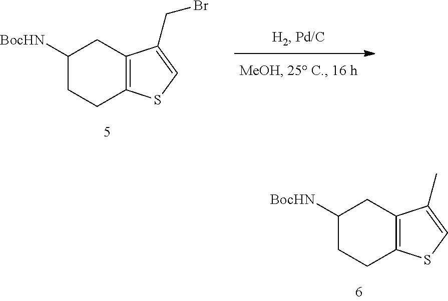 [SMILES: CC(C)(C)OC(=O)NC1CCc2scc(CBr)c2C1 ; Cc1csc2c1CC(NC(=O)OC(C)(C)C)CC2]

To a solution of tert-butyl N-[3-(bromomethyl)-4,5,6,7-tetrahydrobenzothiophen-5-yl]carbamate (5) (1.4 g, 4.04 mmol, 1 eq) in MeOH (15 mL) was added Pd/C (500 mg, 10% Pd) under N₂ atmosphere. The suspension was degassed and purged with H₂ for 3 times. The mixture was stirred under H₂ (15 Psi) at 25° C. for 16 h. The reaction mixture was filtered and the cake was washed with MeOH (20 mL). The combined organic phases were concentrated under vacuum to give a residue. The residue was purified by flash silica gel chromatography (ISCO®; 4 g SepaFlash® Silica Flash Column, eluent 0~6% Ethyl acetate/Petroleum ether gradient @40 mL/min) to yield tert-butyl N-(3-methyl-4,5,6,7-tetrahydrobenzothiophen-5-yl)carbamate (6) (1 g, 3.74 mmol) as a white solid. ¹H NMR (400 MHz, CHLOROFORM-d) δ 6.75 (s, 1H), 4.73-4.62 (m, 1H), 4.07 (br d, J=2.4 Hz, 1H), 2.91-2.86 (m, 2H), 2.35 (br dd, J=7.2, 16.2 Hz, 1H), 2.12 (s, 3H), 2.06 (br d, J=2.8 Hz, 1H), 1.92-1.84 (m, 1H), 1.49 (s, 9H). LCMS: m/z [M+H-tBu]⁺=212.0

### Procedure for preparation of tert-butyl N-methyl-N-(3-methyl-4,5,6,7-tetrahydrobenzothiophen-5-yl) carbamate (7)

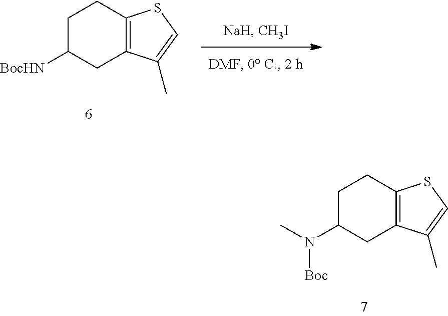 [SMILES: Cc1csc2c1CC(NC(=O)OC(C)(C)C)CC2 ; Cc1csc2c1CC(N(C)C(=O)OC(C)(C)C)CC2]

To a mixture of tert-butyl N-(3-methyl-4,5,6,7-tetrahydrobenzothiophen-5-yl) carbamate (6) (460 mg, 1.72 mmol, 1 eq) in DMF (5 mL) was added NaH (103.19 mg, 2.58 mmol, 60% suspension in mineral oil, 1.5 eq) in one portion at 0° C. under N₂. The mixture was stirred at 0° C. for 1 hour. Then to the mixture was added MeI (366.28 mg, 2.58 mmol, 160.65 μL, 1.5 eq) at 0° C. and stirred at 0° C. for 1 hours. The mixture was quenched with saturated ammonium chloride aqueous solution (10 mL). The mixture was extracted with EtOAc (20 mL*3). The combined organic phase was washed with brine (20 mL*3), dried over Na₂SO₄, filtered and concentrated at reduced pressure to give a residue. The residue was purified by prep-HPLC (column: YMC-Actus Triart C18 150*30 mm*7 μm; mobile phase: [water (FA)-can]; B %: 68%-98%, 10 min) to afford tert-butyl N-methyl-N-(3-methyl-4,5,6,7-tetrahydrobenzothiophen-5-yl) carbamate (7) (240 mg, 852.84 μmol, 49.58% yield) as a white solid. LCMS: m/z [M+H]⁺=226.2.

### Procedure for preparation of N, 3-dimethyl-4,5,6,7-tetrahydrobenzothiophen-5-amine hydrochloride (8)

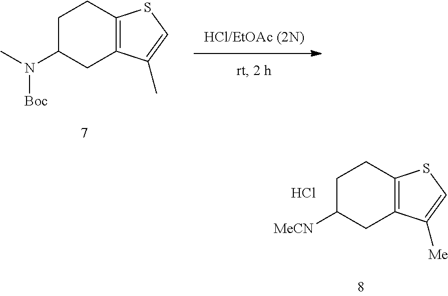

To a mixture of tert-butyl N-methyl-N-(3-methyl-4,5,6,7-tetrahydrobenzothiophen-5-yl) carbamate (7) (210 mg, 746.23 μmol, 1 eq) in EtOAc (1 mL) was added HCl/EtOAc (1 mL) under N₂. The mixture was stirred at 25° C. for 2 hours. The reaction mixture was concentrated under reduced pressure to give a residue. The crude product was triturated with ethyl acetate at 25° C. for 30 min. Compound racemic N, 3-dimethyl-4,5,6,7-tetrahydrobenzothiophen-5-amine hydrochloride (8) (150 mg, 688.84 μmol, 92.31% yield) was obtained as a white solid. LCMS: m/z [M+H]⁺=182.2.

### Procedure for preparation of N, 3-dimethyl-4,5,6,7-tetrahydrobenzothiophen-5-amine hydrochloride enantiomer 1 (69) and enantiomer 2 (70)

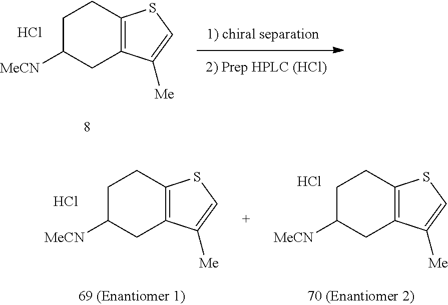

Racemic N, 3-dimethyl-4, 5, 6, 7-tetrahydrobenzothiophen-5-amine hydrochloride (8) was separated to enantiomers (column: ChiralPak IH, 250*50 mm, 10 μm; mobile phase: [0.1% NH₃H₂O MEOH]; B %: 30%-30%, 3.5 min) and each enantiomer was further purified by prep-HPLC (column: Welch Xtimate C18 150*25 mm*5 μm; mobile phase: [water (HCl)-ACN]; B %: 3%-33%, 8 min). N, 3-dimethyl-4,5,6,7-tetrahydrobenzothiophen-5-amine hydrochloride enantiomer 1 (69) (35.9 mg, 164.86 μmol, 37.36% yield) was obtained as a white solid. LCMS: m/z [M+H]⁺=182.2. H NMR (400 MHz, Methanol-d4, 299 K) δ (ppm)=6.83 (s, 1H), 3.57-3.47 (m, 1H), 3.05 (dd, J=5.3, 15.6 Hz, 1H), 3.00-2.85 (m, 2H), 2.81 (s, 3H), 2.57 (br dd, J=9.2, 15.7 Hz, 1H), 2.38-2.28 (m, 1H), 2.13 (s, 3H), 2.05-1.90 (m, 1H). Compound N, 3-dimethyl-4,5,6,7-tetrahydrobenzothiophen-5-amine hydrochloride enantiomer 2 (70) (41.7 mg, 43.40% yield) was obtained also as a white solid. LCMS: m/z [M+H]⁺=182.2. ¹H NMR (400 MHz, Methanol-d4, 299 K) δ (ppm)=6.83 (s, 1H), 3.57-3.47 (m, 1H), 3.05 (dd, J=5.3, 15.7 Hz, 1H), 3.00-2.85 (m, 2H), 2.81 (s, 3H), 2.63-2.52 (m, 1H), 2.38-2.27 (m, 1H), 2.12 (s, 3H), 2.04-1.89 (m, 1H)

### Synthetic scheme for preparation of 3-fluoro-N-methyl-4,5,6,7-tetrahydro-2-benzothiophen-5-amine hydrochloride enantiomer 1 (73) and enantiomer 2 (74); 1-fluoro-N-methyl-4,5,6,7-tetrahydro-2-benzothiophen-5-amine hydrochloride enantiomer 1 (75) and enantiomer 2 (76); 1,3-difluoro-N-methyl-4,5,6,7-tetrahydro-2-benzothiophen-5-amine hydrochloride enantiomer 1 (77) and enantiomer 2 (78)

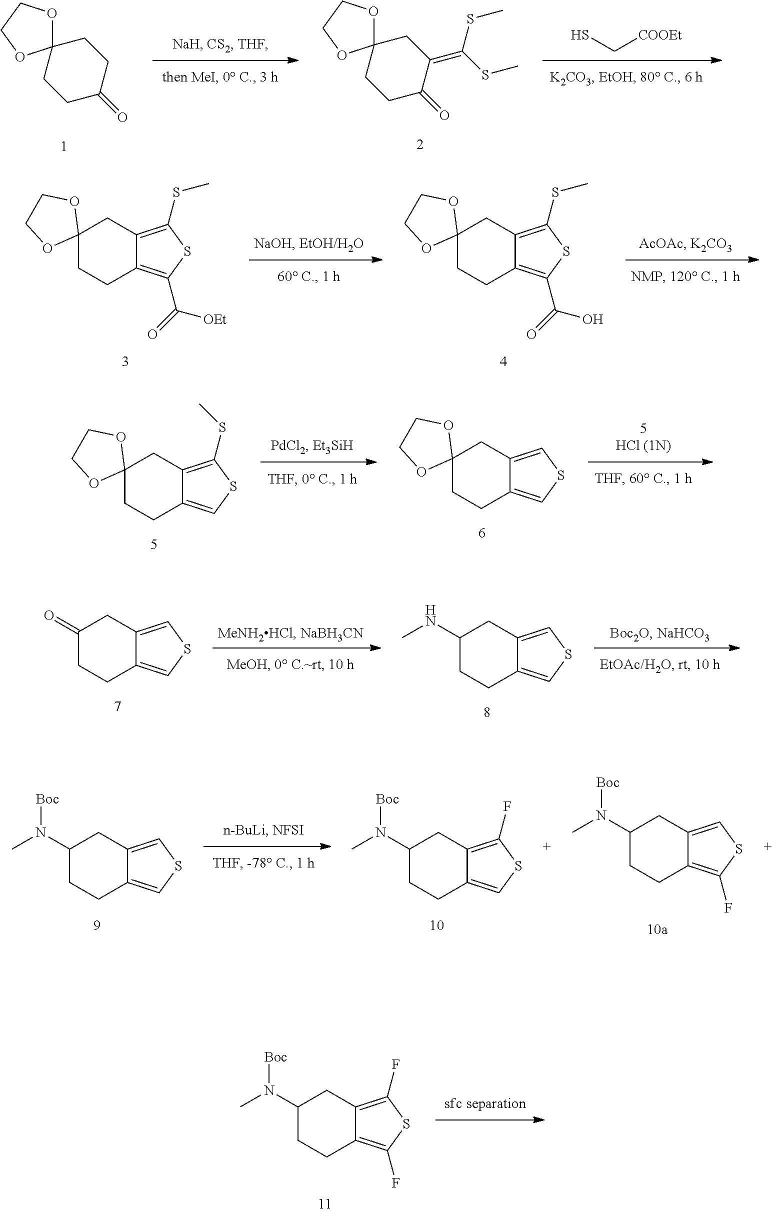

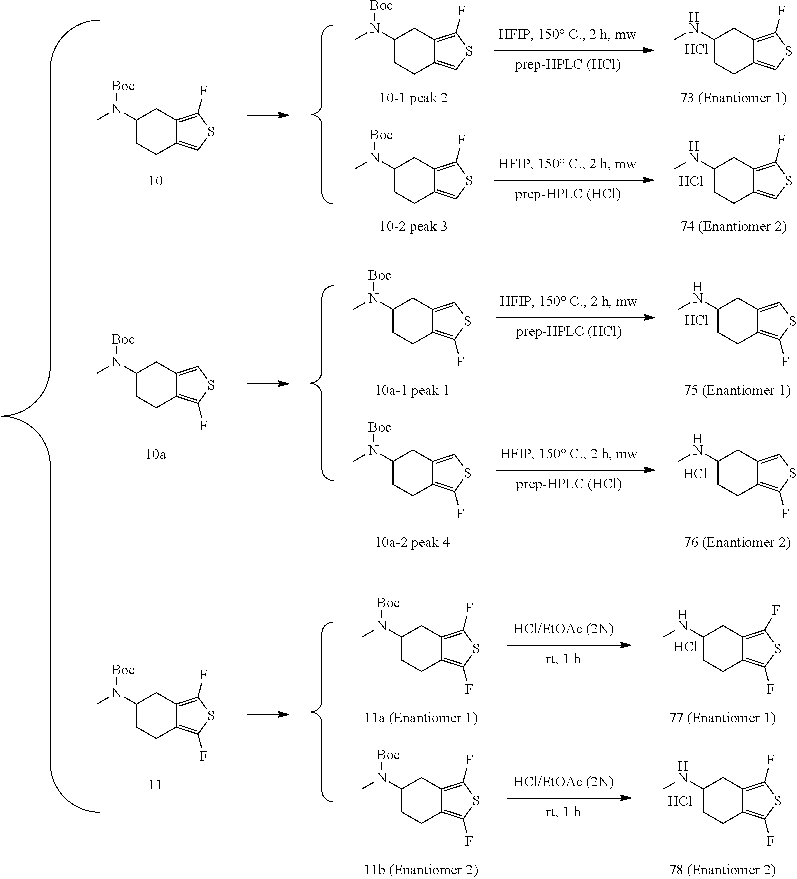

### Procedure for preparation of 7-[bis(methylsulfanyl)methylene]-1,4-dioxaspiro[4.5]decan-8-one (2)

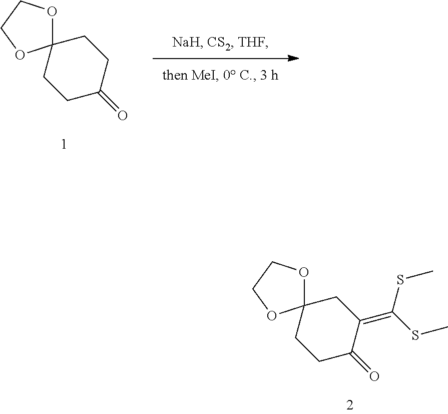 [SMILES: O=C1CCC2(CC1)OCCO2 ; CSC(SC)=C1CC2(CCC1=O)OCCO2]

NaH (30.7 g, 768, mmol, 60% dispersion in mineral oil, 1.2 eq) was added slowly to THF (600 mL) under N₂. The reaction was cooled to 0° C., and added 1,4-dioxaspiro[4.5]decan-8-one (1) (100 g, 640 mmol, 1 eq) slowly under N₂. The mixture was stirred at 0° C. for 0.5 h under N₂, then was added a solution of CS₂ (51.2 g, 672 mmol, 40.6 mL, 1.05 eq) in THF (100 mL) dropwise under N₂. The mixture was stirred at 0° C. for 0.5 h, a solution of MeI (199 g, 1.41 μmol, 87.6 mL, 2.2 eq) in THF (100 mL) was added to the mixture at 0° C. The mixture was stirred at 0° C. for 2 h. The reaction mixture was added to aq. sat. NH₄Cl (1 L) at 0° C., and then diluted with H₂O 500 mL and extracted with EtOAc (800 mL*2). The combined organic layers were washed with brine (800 mL), dried over anhydrous Na₂SO₄, filtered and concentrated under reduced pressure to give a residue. The residue was purified by reverse-phase HPLC (0.1% FA condition) to give a yellow oil, then was purified by flash silica gel chromatography (ISCO®; 220 g SepaFlash® Silica Flash Column, eluent 0~30% Ethyl acetate/Petroleum ether gradient @90 mL/min). Compound 7-[bis(methylsulfanyl)methylene]-1,4-dioxaspiro[4.5]decan-8-one (2) (66.8 g, 256.56 mmol, 40.07% yield) was obtained as a yellow solid. ¹H NMR (400 MHz, CHLOROFORM-d) δ=4.08-3.97 (m, 4H), 3.11 (s, 2H), 2.60 (t, J=7.2 Hz, 2H), 2.38 (d, J=16.0 Hz, 6H), 2.04 (t, J=7.1 Hz, 2H)

### Procedure for preparation of ethyl 3′-methylsulfanylspiro[1,3-dioxolane-2,5′-6,7-dihydro-4H-2-benzothiophene]-1′-carboxylate (3)

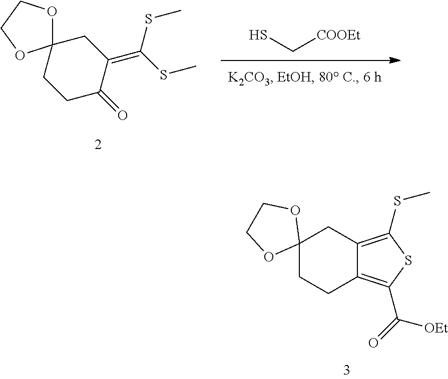 [SMILES: CSC(SC)=C1CC2(CCC1=O)OCCO2 ; CCOC(=O)CS ; CCOC(=O)c1sc(SC)c2c1CCC1(C2)OCCO1]

To a solution of 7-[bis(methylsulfanyl)methylene]-1,4-dioxaspiro[4.5]decan-8-one (2) (66.8 g, 256 mmol, 1 eq) in EtOH (700 mL) was added K₂CO₃ (42.55 g, 307.87 mmol, 1.2 eq) and ethyl 2-sulfanylacetate (37.9 g, 315 mmol, 34.5 mL, 1.23 eq). The mixture was stirred at 80° C. for 6 hr. The reaction mixture was concentrated under reduced pressure to remove EtOH. The residue was diluted with H₂O (600 mL) and extracted with EtOAc (600 mL*2). The combined organic layers were washed with aq. sat. NaCl (400 mL), dried over anhydrous Na₂SO₄, filtered and concentrated under reduced pressure to give a residue. The residue was purified by flash silica gel chromatography (ISCO®; 330 g SepaFlash® Silica Flash Column, eluent 0~20% Ethyl acetate/Petroleum ether gradient @90 mL/min). Compound ethyl 3′-methylsulfanylspiro[1,3-dioxolane-2,5′-6,7-dihydro-4H-2-benzothiophene]-1′-carboxylate (3) (59.4 g, 188.92 mmol, 73.64% yield) was obtained as a yellow solid. ¹H NMR (400 MHz, CHLOROFORM-d) δ=4.30 (q, J=7.1 Hz, 2H), 4.08-3.99 (m, 4H), 3.20 (t, J=6.8 Hz, 2H), 2.78 (s, 2H), 2.51 (s, 3H), 1.93 (t, J=6.8 Hz, 2H), 1.35 (t, J=7.2 Hz, 3H)

### Procedure for preparation of 3′-methylsulfanylspiro[1,3-dioxolane-2,5′-6,7-dihydro-4H-2-benzothiophene]-1′-carboxylic acid (4)

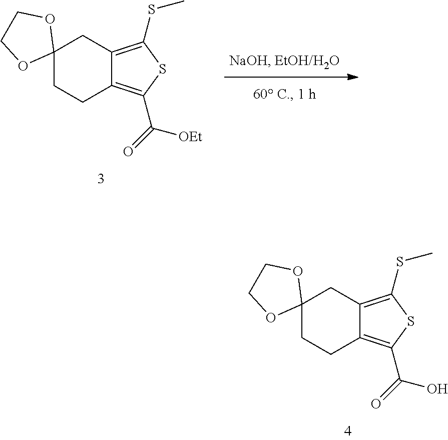 [SMILES: CCOC(=O)c1sc(SC)c2c1CCC1(C2)OCCO1 ; CSc1sc(C(=O)O)c2c1CC1(CC2)OCCO1]

To a solution of ethyl 3′-methylsulfanylspiro[1,3-dioxolane-2,5′-6,7-dihydro-4H-2-benzothiophene]-1′-carboxylate (3) (40.7 g, 129.44 mmol, 1 eq) in EtOH (400 mL) and H₂O (100 mL) was added NaOH (15.53 g, 388.33 mmol, 3 eq). The mixture was stirred at 60° C. for 1 hr. The reaction mixture was concentrated under reduced pressure to remove EtOH. The residue was diluted with H₂O 500 mL. Added aq. HCl (3 N) to adjust pH=6 at 0° C. The solid formed filtered and triturated with H₂O (200 mL) at 25° C. for 10 mins. The mixture was filtered and washed with Petroleum ether (200 mL), dried to give 3′-methylsulfanylspiro[1,3-dioxolane-2,5′-6,7-dihydro-4H-2-benzothiophene]-1′-carboxylic acid (4) (35 g, 122.22 mmol, 94.42% yield) as an off-white solid. ¹H NMR (400 MHz, DMSO-d6) δ=3.98-3.89 (m, 4H), 3.00 (br t, J=6.7 Hz, 2H), 2.65 (s, 2H), 2.51 (s, 3H), 1.83 (br t, J=6.7 Hz, 2H).

### Procedure for preparation of 1′-methylsulfanylspiro[1,3-dioxolane-2,6′-5,7-dihydro-4H-2-benzothiophene](5)

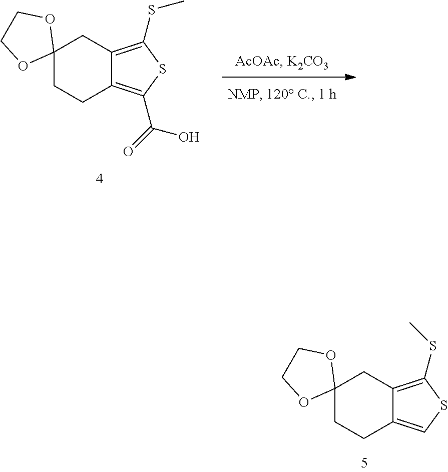 [SMILES: CSc1sc(C(=O)O)c2c1CC1(CC2)OCCO1 ; CSc1scc2c1CC1(CC2)OCCO1]

To a solution of 3′-methylsulfanylspiro[1,3-dioxolane-2,5′-6,7-dihydro-4H-2-benzothiophene]-1′-carboxylic acid (4) (26.1 g, 91.14 mmol, 1 eq) in NMP (250 mL) was added AgOAc (1.52 g, 9.11 mmol, 466.65 uL, 0.1 eq) and K₂CO₃ (37.79 g, 273.43 mmol, 3 eq). The mixture was stirred at 120° C. for 1 hr under N₂. The reaction mixture was filtered, then diluted with H₂O 800 mL and extracted with EtOAc (300 mL*2). The combined organic layers were washed with brine (300 mL), dried over anhydrous Na₂SO₄, filtered and concentrated under reduced pressure to give a residue. The residue was purified by flash silica gel chromatography (ISCO®; 120 g SepaFlash® Silica Flash Column, eluent 0~20% Ethyl acetate/Petroleum ether gradient @70 mL/min) to give 1′-methylsulfanylspiro[1,3-dioxolane-2,6′-5,7-dihydro-4H-2-benzothiophene](5) (21.2 g, 87.47 mmol, 95.98% yield) as a yellowish oil. ¹H NMR (400 MHz, CHLOROFORM-d) δ=6.93 (s, 1H), 4.10-4.00 (m, 4H), 2.95-2.84 (m, 4H), 2.37 (s, 3H), 1.92 (t, J=6.7 Hz, 2H)

### Procedure for preparation of spiro[1,3-dioxolane-2,6′-5,7-dihydro-4H-2-benzothiophene](6)

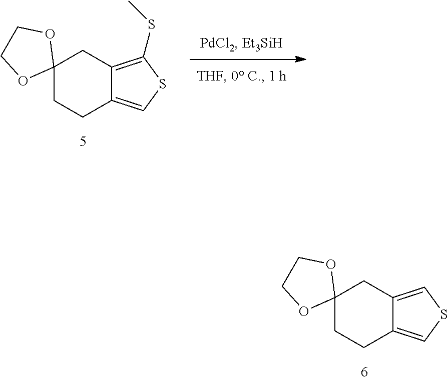

To a solution of 1′-methylsulfanylspiro[1,3-dioxolane-2,6′-5,7-dihydro-4H-2-benzothiophene](5) (10 g, 41.3 mmol, 1 eq) in THF (100 mL) was added PdCl₂ (585 mg, 3.30 mmol, 0.08 eq) and Et₃SiH (7.20 g, 61.9 mmol, 9.89 mL, 1.5 eq) at 0° C. The mixture was stirred at 0° C. for 1 hr. The reaction mixture was quenched with H₂O (50 ml) and extracted with EtOAc (50 mL*2). The combined organic phase was washed with brine (50 mL), dried over anhydrous sodium sulfate, filtered and concentrated under vacuum to give a residue. The residue was purified by flash silica gel chromatography (ISCO®; 40 g SepaFlash® Silica Flash Column, eluent 0~15% Ethyl acetate/Petroleum ether gradient @40 mL/min) to afford spiro[1,3-dioxolane-2,6′-5,7-dihydro-4H-2-benzothiophene](6) (8.4 g, crude) as a yellow oil. ¹H NMR (400 MHz, CHLOROFORM-d) δ=6.96-6.85 (m, 2H), 4.03 (qd, J=1.9, 4.1 Hz, 4H), 2.98-2.88 (m, 4H), 1.95 (t, J=6.8 Hz, 2H) LCMS: m/z [M+H]⁺=197.0

### Procedure for preparation of 6,7-dihydro-4H-2-benzothiophen-5-one (7)

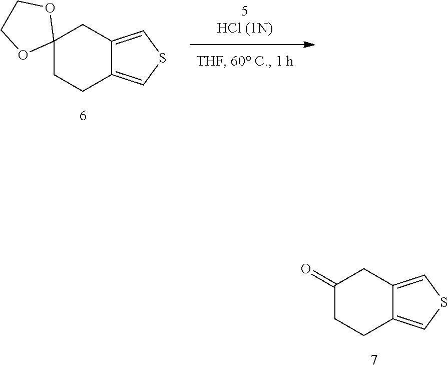 [SMILES: c1scc2c1CCC1(C2)OCCO1 ; O=C1CCc2cscc2C1]

To a solution of spiro[1,3-dioxolane-2,6′-5,7-dihydro-4H-2-benzothiophene](6) (8.4 g, 42.8 mmol, 1 eq) in THF (100 mL) was added HCl (1 M, 100 mL, 2.34 eq). The mixture was stirred at 60° C. for 1 hr. The reaction mixture was poured into aq. sat.Na₂CO₃ (50 mL) and extracted with EtOAc (50 mL*2). The combined organic layers were washed with brine (30 mL), dried over anhydrous Na₂SO₄, filtered and concentrated under reduced pressure to give 6,7-dihydro-4H-2-benzothiophen-5-one (7) (7.3 g, crude) as a yellowish oil. ¹H NMR (400 MHz, CHLOROFORM-d) δ=7.05-6.98 (m, 2H), 3.59 (s, 2H), 3.03 (t, J=6.8 Hz, 2H), 2.62-2.56 (m, 2H)

### Procedure for preparation of N-methyl-4,5,6,7-tetrahydro-2-benzothiophen-5-amine (8)

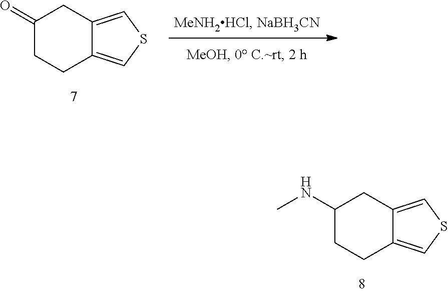

To the mixture of 6,7-dihydro-4H-2-benzothiophen-5-one (7) (7.3 g, 48.0 mmol, 1 eq, crude) and methanamine hydrochloride (12.9 g, 192 mmol, 4 eq) in MeOH (150 mL) was added NaBH₃CN (18.1 g, 287 mmol, 6 eq) at 0° C. The resulting mixture was stirred at 25° C. for 2 hr. The reaction mixture was poured into H₂O (10 mL) and was concentrated in vacuo to give N-methyl-4,5,6,7-tetrahydro-2-benzothiophen-5-amine (8) (7 g, crude). LCMS: m/z [M+H]⁺=168.2.

### Procedure for preparation of tert-butyl N-methyl-N-(4,5,6,7-tetrahydro-2-benzothiophen-5-yl)carbamate (9)

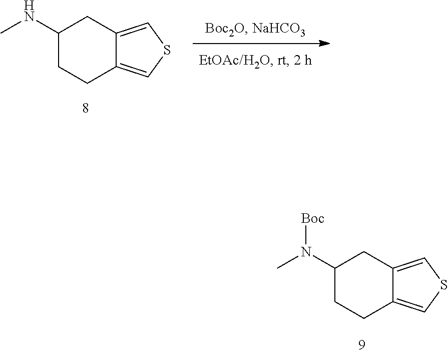 [SMILES: CNC1CCc2cscc2C1 ; CN(C(=O)OC(C)(C)C)C1CCc2cscc2C1]

To a mixture of N-methyl-4,5,6,7-tetrahydro-2-benzothiophen-5-amine (8) (7 g, 41.8 mmol, 1 eq, crude) in EtOAc (100 mL) and H₂O (100 mL) was added (Boc)₂O (18.2 g, 83.7 mmol, 19.2 mL, 2 eq) NaHCO₃ (17.6 g, 209 mmol, 8.14 mL, 5 eq). The mixture was stirred at 25° C. for 2 hr. The reaction mixture was extracted with EtOAc (100 mL*2). The combined organic layers were washed with brine (60 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0-5% Ethyl acetate/Petroleum ether gradient @40 mL/min) to give tert-butyl N-methyl-N-(4,5,6,7-tetrahydro-2-benzothiophen-5-yl)carbamate (9) (3 g, 11.2 mmol) as a light yellow oil. ¹H NMR (400 MHz, CHLOROFORM-d) δ=6.88 (br s, 2H), 4.49-4.19 (m, 1H), 3.03-2.86 (m, 2H), 2.84-2.71 (m, 5H), 1.94-1.78 (m, 2H), 1.48 (s, 9H). LCMS: m/z [M+H-Boc]⁺=168.1

### Procedure for preparation of tert-butyl N-(3-fluoro-4,5,6,7-tetrahydro-2-benzothiophen-5-yl)-N-methyl-carbamate (10); tert-butyl N-(1-fluoro-4,5,6,7-tetrahydro-2-benzothiophen-5-yl)-N-methyl-carbamate (10a) and tert-butyl N-(1,3-difluoro-4,5,6,7-tetrahydro-2-benzothiophen-5-yl)-N-methyl-carbamate (11)

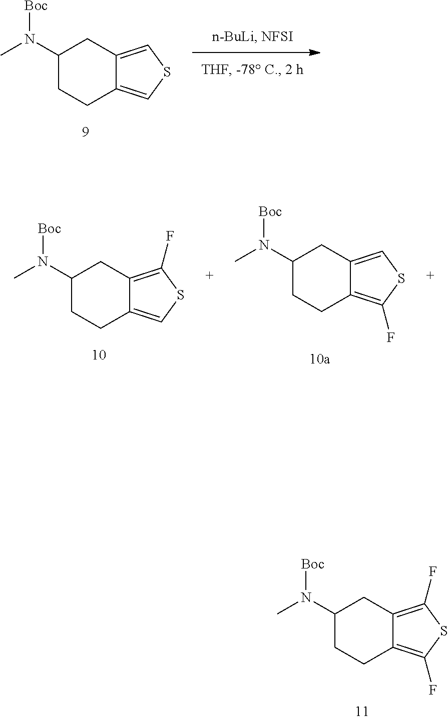 [SMILES: CN(C(=O)OC(C)(C)C)C1CCc2cscc2C1 ; CN(C(=O)OC(C)(C)C)C1CCc2csc(F)c2C1 ; CN(C(=O)OC(C)(C)C)C1CCc2c(csc2F)C1 ; CN(C(=O)OC(C)(C)C)C1CCc2c(F)sc(F)c2C1]

To a solution of tert-butyl N-methyl-N-(4,5,6,7-tetrahydro-2-benzothiophen-5-yl) carbamate (9) (3 g, 11.2 mmol, 1 eq) in THF (30 mL) was added n-BuLi (2.5 M, 6.73 mL, 1.5 eq) dropwise at −78° C. After addition, the mixture was stirred at −78° C. for 0.5 hr, and then NFSI (4.60 g, 14.6 mmol, 1.3 eq) in THF (10 mL) was added dropwise at −78° C. The resulting mixture was stirred at −78° C. for 1.5 hr. The reaction mixture was quenched by addition aq. sat.NH₄Cl 20 mL and extracted with EtOAc (30 mL*3). The combined organic layers were washed with brine (30 mL), dried over anhydrous Na₂SO₄, filtered and concentrated under reduced pressure to give a residue. The residue was purified by prep-HPLC (column: Kromasil Eternity XT 250*80 mm*10 μm; mobile phase: [water(NH₄HCO₃)-ACN]; gradient:55%-85% B over 20 min) to give a mixture (1.7 g, 5.96 mmol) of tert-butyl N-(3-fluoro-4,5,6,7-tetrahydro-2-benzothiophen-5-yl)-N-methyl-carbamate (10), tert-butyl N-(1-fluoro-4,5,6,7-tetrahydro-2-benzothiophen-5-yl)-N-methyl-carbamate (10a) as a white solid, and compound tert-butyl N-(1,3-difluoro-4,5,6,7-tetrahydro-2-benzothiophen-5-yl)-N-methyl-carbamate (11) (350 mg) as a white solid.

### Procedure for preparation of tert-butyl N-(3-fluoro-4,5,6,7-tetrahydro-2-benzothiophen-5-yl)-N-methyl-carbamate enantiomer 1 (10-1) and enantiomer 2 (10-2) and tert-butyl N-(1-fluoro-4,5,6,7-tetrahydro-2-benzothiophen-5-yl)-N-methyl-carbamate enantiomer 1 (10a-1) and enantiomer 2 (10a-2)

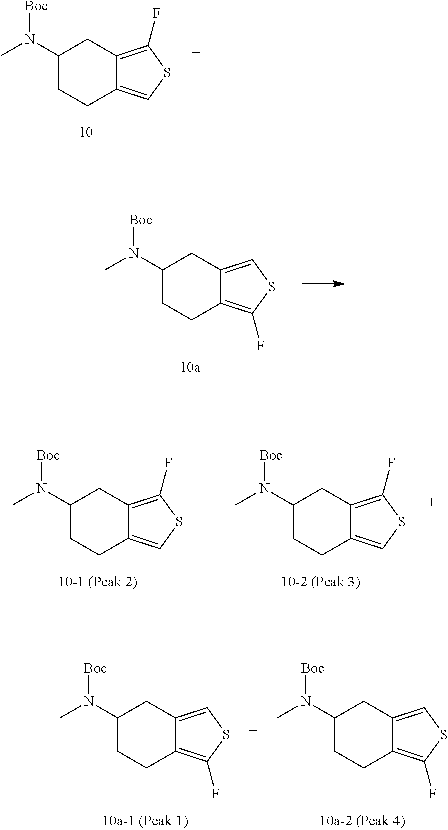 [SMILES: CN(C(=O)OC(C)(C)C)C1CCc2csc(F)c2C1 ; CN(C(=O)OC(C)(C)C)C1CCc2c(csc2F)C1 ; CN(C(=O)OC(C)(C)C)C1CCc2csc(F)c2C1 ; CN(C(=O)OC(C)(C)C)C1CCc2csc(F)c2C1 ; CN(C(=O)OC(C)(C)C)C1CCc2c(csc2F)C1 ; CN(C(=O)OC(C)(C)C)C1CCc2c(csc2F)C1]

1.6 g of the mixture of 10 and 10a was separated by SFC (column: DAICEL CHIRALPAK AD (250 mm*30 mm, 10 μm); mobile phase: [Hexane-EtOH (0.1% NH₃·H₂O)]; B %:10%, isocratic elution mode) to give part 1 (a mixture of 10a-1 and 10-1), part 2 (a mixture of 10-2 and 10a-2).

The mixture of 10a-1 and 10-1 was separated by SFC (column: DAICEL CHIRALPAK IG (250 mm*50 mm, 10 μm); mobile phase: [Hexane-EtOH (0.1% NH₃·H₂O)]; B %:10%, isocratic elution mode) to give 10a-1 (180 mg, 630.75 μmol, 11.25% yield) as a yellow oil and 10-1, (80 mg, 280.33 μmol, 5.00% yield) as a yellow oil. The mixture of 10-2 and 10a-2 was separated by SFC (column: DAICEL CHIRALPAK IG (250 mm*50 mm, 10 μm); mobile phase: [CO₂-EtOH hexane(0.1% NH₃H₂O)]; B %:10%, isocratic elution mode) to give compound 10-2 (160 mg, 560.66 μmol, 10.00% yield) as a yellow oil and 10a-2 (260 mg, 911.08 μmol, 16.25% yield) as a yellow oil.

### Procedure for preparation of 3-fluoro-N-methyl-4,5,6,7-tetrahydro-2-benzothiophen-5-amine hydrochloride enantiomer 1 (73)

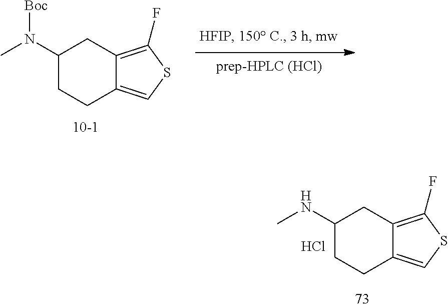 [SMILES: CN(C(=O)OC(C)(C)C)C1CCc2csc(F)c2C1 ; CNC1CCc2csc(F)c2C1 ; Cl]

A solution of tert-butyl N-(3-fluoro-4,5,6,7-tetrahydro-2-benzothiophen-5-yl)-N-methyl-carbamate peak 2 (10-1) (80 mg, 280.33 μmol, 1 eq) in 1,1,1,3,3,3-hexafluoropropan-2-ol (2 mL) was stirred at 150° C. under microwave heating for 3 hr. The reaction mixture was concentrated under reduced pressure to remove 1,1,1,3,3,3-hexafluoropropan-2-ol. The residue was purified by prep-HPLC (HCl condition:Welch Xtimate C18 150*25 mm*5 μm; mobile phase: [water(HCl)-ACN]; gradient:0%-30% B over 9 min). Compound 3-fluoro-N-methyl-4,5,6,7-tetrahydro-2-benzothiophen-5-amine hydrochloride enantiomer 1 (73) (9.59 mg, 43.25 μmol, 15.43% yield) was obtained as a white solid. ¹H NMR (400 MHz, METHANOL-d₄) δ=6.60-6.48 (m, 1H), 3.43 (ddt, J=2.8, 5.0, 10.3 Hz, 1H), 3.21 (dd, J=4.9, 15.7 Hz, 1H), 2.98-2.86 (m, 1H), 2.82-2.59 (m, 5H), 2.31-2.19 (m, 1H), 1.83 (dtd, J=5.8, 10.7, 13.0 Hz, 1H). LCMS: m/z [M+H]⁺=186.3

### Procedure for preparation of 3-fluoro-N-methyl-4,5,6,7-tetrahydro-2-benzothiophen-5-amine hydrochloride enantiomer 2 (74)

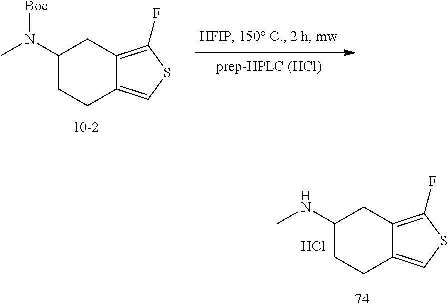 [SMILES: CN(C(=O)OC(C)(C)C)C1CCc2csc(F)c2C1 ; CNC1CCc2csc(F)c2C1 ; Cl]

A solution of tert-butyl N-(3-fluoro-4,5,6,7-tetrahydro-2-benzothiophen-5-yl)-N-methyl-carbamate (peak 3, 10-2) (160 mg, 560.66 μmol, 1 eq) in 1,1,1,3,3,3-hexafluoropropan-2-ol (3 mL) was stirred at 150° C. for 2 hr under microwave heating. The reaction mixture was concentrated under reduced pressure to remove 1,1,1,3,3,3-hexafluoropropan-2-ol. The residue was purified by prep-HPLC (HCl condition: Welch Xtimate C18 150*25 mm*5 μm; mobile phase: [water(HCl)-ACN]; gradient:0%-30% B over 9 min). Compound 3-fluoro-N-methyl-4,5,6,7-tetrahydro-2-benzothiophen-5-amine hydrochloride enantiomer 2 (74) (53.23 mg, 240.08 μmol, 42.82% yield) was obtained as a white solid. ¹H NMR (400 MHz, METHANOL-d₄) δ=6.58-6.51 (m, 1H), 3.42 (dddd, J=2.4, 5.1, 7.7, 10.2 Hz, 1H), 3.21 (br dd, J=4.9, 15.6 Hz, 1H), 2.96-2.86 (m, 1H), 2.81-2.59 (m, 5H), 2.30-2.20 (m, 1H), 1.83 (dtd, J=5.8, 10.7, 12.9 Hz, 1H), LCMS: m/z [M+H]⁺=186.3

### Procedure for preparation of 1-fluoro-N-methyl-4,5,6,7-tetrahydro-2-benzothiophen-5-amine hydrochloride enantiomer 1 (75)

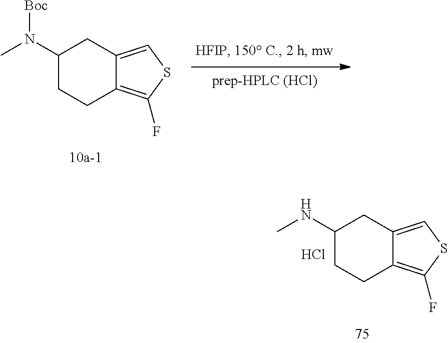 [SMILES: CN(C(=O)OC(C)(C)C)C1CCc2c(csc2F)C1 ; CNC1CCc2c(csc2F)C1 ; Cl]

A solution of tert-butyl N-(1-fluoro-4,5,6,7-tetrahydro-2-benzothiophen-5-yl)-N-methyl-carbamate (peak1, 10a-1) (180 mg, 630.75 μmol, 1 eq) in 1,1,1,3,3,3-hexafluoropropan-2-ol (3 mL) was stirred at 150° C. for 2 hr under microwave heating. The reaction mixture was concentrated under reduced pressure to remove 1,1,1,3,3,3-hexafluoropropan-2-ol. The residue was purified by prep-HPLC (HCl condition:Welch Xtimate C18 150*25 mm*5 μm; mobile phase: [water(HCl)-ACN]; gradient:0%-30% B over 9 min). Compound 1-fluoro-N-methyl-4,5,6,7-tetrahydro-2-benzothiophen-5-amine hydrochloride enantiomer 1 (75) (47.06 mg, 254.02 μmol, 40.27% yield) was obtained as a white solid. ¹H NMR (400 MHz, METHANOL-d₄) δ=6.51-6.43 (m, 1H), 3.45 (ddt, J=2.8, 5.5, 10.4 Hz, 1H), 3.25 (br dd, J=5.4, 15.8 Hz, 1H), 2.94-2.85 (m, 1H), 2.82-2.57 (m, 5H), 2.29-2.20 (m, 1H), 1.83 (dtd, J=5.4, 11.1, 12.8 Hz, 1H); LCMS: m/z [M+H]⁺=186.3

### Procedure for preparation of 1-fluoro-N-methyl-4,5,6,7-tetrahydro-2-benzothiophen-5-amine hydrochloride enantiomer 2 (76)

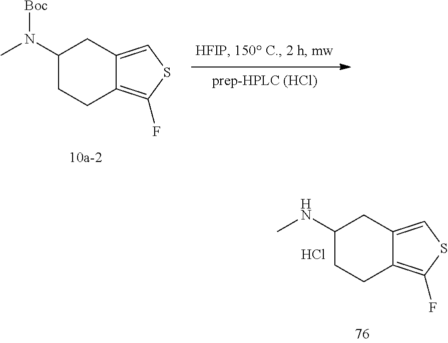 [SMILES: CN(C(=O)OC(C)(C)C)C1CCc2c(csc2F)C1 ; CNC1CCc2c(csc2F)C1 ; Cl]

The tert-butyl N-(1-fluoro-4,5,6,7-tetrahydro-2-benzothiophen-5-yl)-N-methyl-carbamate (peak4 10a-2) (100 mg, 350.41 μmol, 1 eq) was taken up into a microwave tube in 1,1,1,3,3,3-hexafluoropropan-2-ol (2 mL). The sealed tube was heated in a microwave at 150° C. for 2 hr. Mixture filtered and concentrated under vacuum to give a residue. The residue was purified by prep-HPLC (column: Welch Xtimate C18 150*25 mm*5 μm; mobile phase: [water(HCl)-ACN]; gradient:0%-30% B over 9 min). Compound 1-fluoro-N-methyl-4,5,6,7-tetrahydro-2-benzothiophen-5-amine hydrochloride enantiomer 2 (76) (30 mg, 135.30 μmol, 38.61% yield) was obtained as a white solid. ¹H NMR (400 MHz, METHANOL-d4) δ=6.54-6.41 (m, 1H), 3.45 (ddt, J=2.8, 5.4, 10.4 Hz, 1H), 3.25 (br dd, J=5.4, 15.8 Hz, 1H), 2.94-2.84 (m, 1H), 2.81-2.56 (m, 5H), 2.29-2.18 (m, 1H), 1.82 (dtd, J=5.3, 11.1, 12.8 Hz, 1H). LCMS: m/z [M+H]⁺=186.3

### Procedure for preparation of tert-butyl N-(1,3-difluoro-4,5,6,7-tetrahydro-2-benzothiophen-5-yl)-N-methyl-carbamate enantiomer 1 (11a) and enantiomer 2 (Jib)

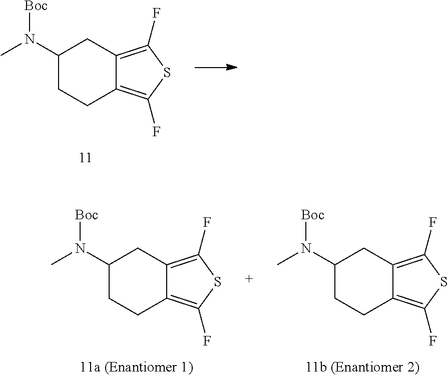 [SMILES: CN(C(=O)OC(C)(C)C)C1CCc2c(F)sc(F)c2C1 ; CN(C(=O)OC(C)(C)C)C1CCc2c(F)sc(F)c2C1 ; CN(C(=O)OC(C)(C)C)C1CCc2c(F)sc(F)c2C1]

Racemic tert-butyl N-(1,3-difluoro-4,5,6,7-tetrahydro-2-benzothiophen-5-yl)-N-methyl-carbamate 11 was subjected to prep-Chiral-SFC (column: DAICEL CHIRALPAK AD (250 mm*50 mm, 10 μm); mobile phase: [Hexane-EtOH (0.1% NH₃·H₂O)]; B %: 15%-15%, 3.6 min) to afford the 1^{st} enantiomer 11a (RT=0.444 min, 100 mg, 1.03 mmol) and the 2^{nd} enantiomer 11b (RT=0.660 min, 100 mg, 1.42 mmol, 24.01% yield), both as yellow oils.

### Procedure for preparation of 1,3-difluoro-N-methyl-4,5,6,7-tetrahydro-2-benzothiophen-5-amine hydrochloride enantiomer 1 (77)

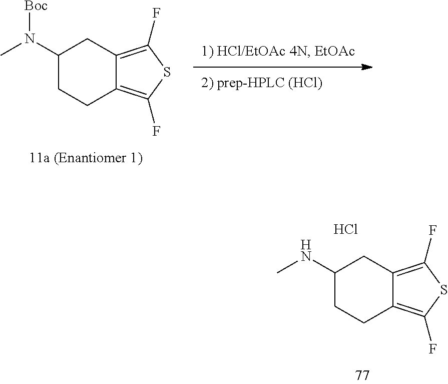 [SMILES: CN(C(=O)OC(C)(C)C)C1CCc2c(F)sc(F)c2C1 ; Cl ; CNC1CCc2c(F)sc(F)c2C1]

To a solution of tert-butyl N-(1,3-difluoro-4,5,6,7-tetrahydro-2-benzothiophen-5-yl)-N-methyl-carbamate enantiomer 1 (11a) (100 mg, 329 μmol, 1 eq) in EtOAc (2 mL) was added HCl/EtOAc (4 M, 2 mL, 24.27 eq). The mixture was stirred at 25° C. for 0.5 hr. The reaction mixture was filtered and the cake was washed with EtOAc (2 mL). The product was purified by prep-HPLC (column: Welch Xtimate C18 150*25 mm*5 μm; mobile phase: [water (HCl)-ACN]; B %: 5%-35%, 8 min) to yield 1, 3-difluoro-N-methyl-4, 5, 6, 7-tetrahydro-2-benzothiophen-5-amine hydrochloride enantiomer 1 (77) (50.7 mg, 209.28 μmol, 63.49% yield) as a white solid. ¹H NMR (400 MHz, METHANOL-d₄) δ=3.49-3.38 (m, 1H), 3.23 (br dd, J=2.9, 16.3 Hz, 1H), 2.97-2.86 (m, 1H), 2.79 (s, 3H), 2.70-2.53 (m, 2H), 2.30-2.17 (m, 1H), 1.87-1.74 (m, 1H). LCMS: m/z [M+H]⁺=204.2.

### Procedure for preparation of 1,3-difluoro-N-methyl-4,5,6,7-tetrahydro-2-benzothiophen-5-amine enantiomer 2 (78)

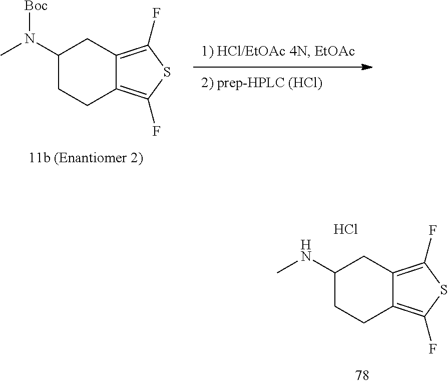 [SMILES: CN(C(=O)OC(C)(C)C)C1CCc2c(F)sc(F)c2C1 ; Cl ; CNC1CCc2c(F)sc(F)c2C1]

To a solution of tert-butyl N-(1,3-difluoro-4,5,6,7-tetrahydro-2-benzothiophen-5-yl)-N-methyl-carbamate enantiomer 2 (11b) (100 mg, 329 μmol, 1 eq) in EtOAc (2 mL) was added HCl/EtOAc (4 M, 2 mL, 24.3 eq). The mixture was stirred at 25° C. for 0.5 hr. The reaction mixture was filtered and the cake was washed with EtOAc (10 mL). The product was purified by prep-HPLC (column: Welch Xtimate C18 150*25 mm*5 μm; mobile phase: [water (HCl)-ACN]; B %: 5%-35%, 8 min) to yield 1, 3-difluoro-N-methyl-4, 5, 6, 7-tetrahydro-2-benzothiophen-5-amine hydrochloride enantiomer 2 (78) (44.2 mg, 183.85 μmol, 55.77% yield) as a white solid. ¹H NMR (400 MHz, METHANOL-d₄) 6=3.47-3.38 (m, 1H), 3.23 (br dd, J=3.1, 15.9 Hz, 1H), 2.97-2.85 (m, 1H), 2.79 (s, 3H), 2.69-2.53 (m, 2H), 2.28-2.18 (m, 1H), 1.87-1.73 (m, 1H). LCMS: m/z [M+H]⁺=204.2.

### Synthetic scheme for the preparation of racemic 2-fluoro-N-methyl-4,5,6,7-tetrahydrobenzothiophen-6-amine hydrochloride (79) and 2-fluoro-N-methyl-4,5,6,7-tetrahydrobenzothiophen-6-amine hydrochloride enantiomer 1 (80) and enantiomer 2 (81)

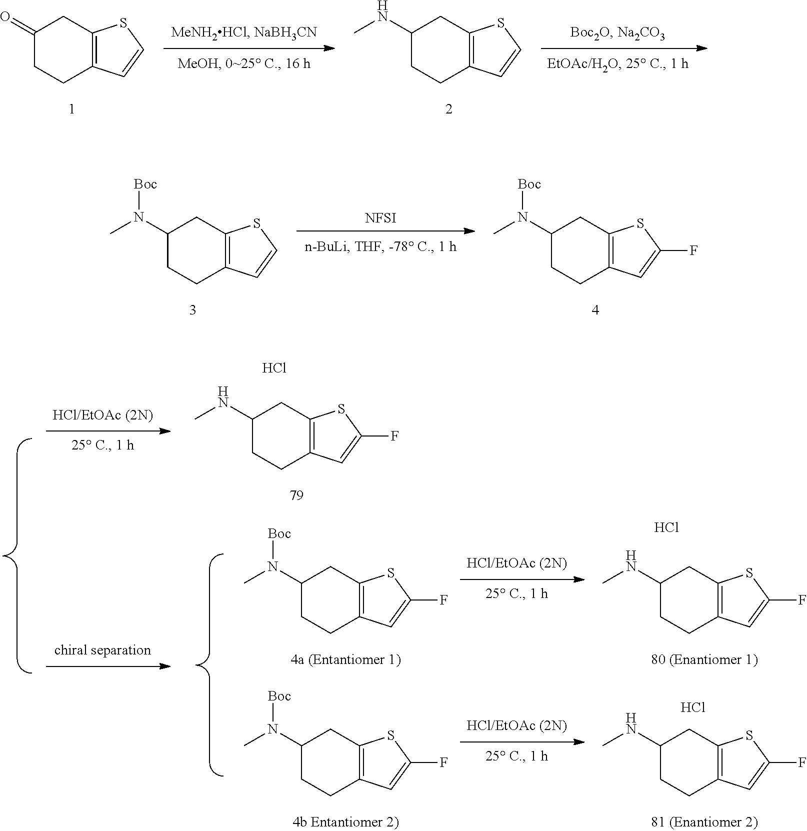

### Procedure for preparation of N-methyl-4,5,6,7-tetrahydrobenzothiophen-6-amine (2)

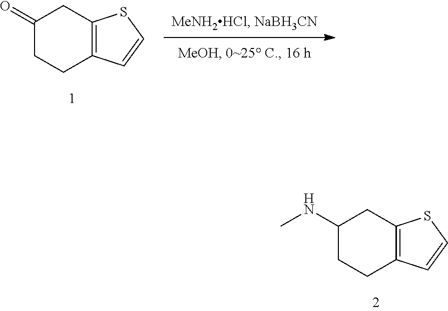

To a solution of 5,7-dihydro-4H-benzothiophen-6-one (1) (4.1 g, 26.94 mmol, 1 eq) and methanamine (7.27 g, 107.74 mmol, 429.90 μL, 4 eq, HCl) in MeOH (20 mL) was added NaBH₃CN (10.16 g, 161.62 mmol, 6 eq) at 0° C. The mixture was stirred at 25° C. for 16 hr. The reaction mixture was quenched by adding 20 mL H₂O. The reaction mixture was concentrated under reduced pressure to remove MeOH, and then diluted with EtOAc 30 mL and extracted with H₂O (20 mL*3). The organic phase was separated, washed with Brine 5 mL, dried over anhydrous sodium sulfate, filtered and concentrated under reduced pressure to give a N-methyl-4,5,6,7-tetrahydrobenzothiophen-6-amine (2) (4.2 g, crude) as a brown liquid.

### Procedure for preparation of tert-butyl N-methyl-N-(4,5,6,7-tetrahydrobenzothiophen-6-yl)carbamate (3)

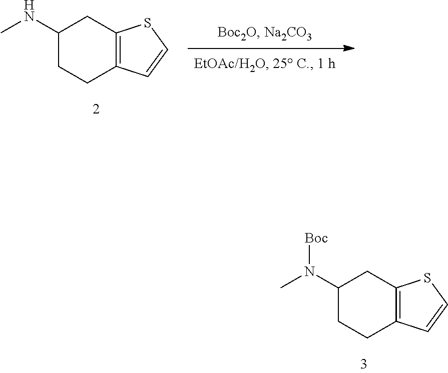 [SMILES: CNC1CCc2ccsc2C1 ; CN(C(=O)OC(C)(C)C)C1CCc2ccsc2C1]

To a solution of N-methyl-4,5,6,7-tetrahydrobenzothiophen-6-amine (2) (4.2 g, 25.11 mmol, 1 eq) in H₂O (10 mL) and EtOAc (10 mL) was added Na₂CO₃ (5.32 g, 50.22 mmol, 2 eq) and Boc₂O (10.96 g, 50.22 mmol, 11.54 mL, 2 eq). The mixture was stirred at 25° C. for 1 hr. To the reaction mixture was added H₂O 10 mL. Mixture then extracted with EtOAc 10 mL. The organic phase was separated, washed with brine 10 ml, dried over anhydrous sodium sulfate, filtered and concentrated under reduced pressure to give a residue. The residue was purified by column chromatography (SiO₂, Petroleum ether/Ethyl acetate=1/0~5/1). Compound tert-butyl N-methyl-N-(4,5,6,7-tetrahydrobenzothiophen-6-yl)carbamate (3) (5.13 g, 19.19 mmol, 76.41% yield) was obtained as a white solid. 1H NMR (400 MHz, CDCl3) δ 7.07 (d, J=5.00 Hz, 1H), 6.74 (d, J=5.13 Hz, 1H), 4.61-4.23 (m, 1H), 2.99-2.64 (m, 7H), 2.05 (s, 1H), 1.99-1.82 (m, 2H), 1.48 (s, 9H)

### Procedure for preparation of tert-butyl N-(2-fluoro-4,5,6,7-tetrahydrobenzothiophen-6-yl)-N-methyl-carbamate (4)

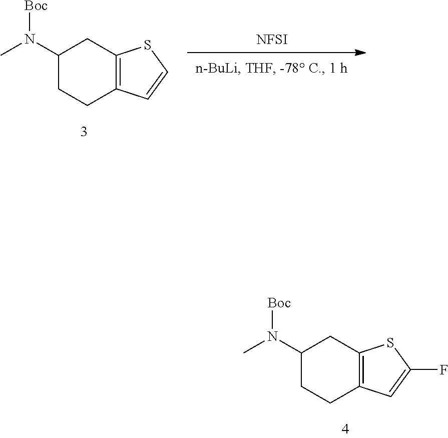 [SMILES: CN(C(=O)OC(C)(C)C)C1CCc2ccsc2C1 ; "NFSI" ; CN(C(=O)OC(C)(C)C)C1CCc2cc(F)sc2C1]

To a solution of tert-butyl N-methyl-N-(4,5,6,7-tetrahydrobenzothiophen-6-yl)carbamate (3) (3 g, 11.22 mmol, 1 eq) in THF (20 mL) was added n-BuLi (2.5 M, 5.39 mL, 1.2 eq) at −78° C. under N₂ stirred for 0.5 h, then was added a solution of NFSI (3.89 g, 12.34 mmol, 1.1 eq) in THF (40 mL). The mixture was stirred at −78° C. for 1 hr. The reaction mixture was quenched by addition aq. sat. NH₄Cl 100 mL at 0° C., and extracted with EtOAc (50 mL*2). The combined organic layers were washed with sat. NaCl (50 mL), dried over anhydrous Na₂SO₄, filtered and concentrated under reduced pressure to give a residue. The residue was purified by column chromatography (SiO₂, Petroleum ether/Ethyl acetate=1/0~5/1), concentrated under reduced pressure to give a residue. The residue was purified by prep-HPLC (basic condition). Compound tert-butyl N-(2-fluoro-4,5,6,7-tetrahydrobenzothiophen-6-yl)-N-methyl-carbamate (4)(1.87 g, 6.57 mmol, 69.41% yield) was obtained as a brown oil.

### Procedure for preparation of racemic 2-fluoro-N-methyl-4,5,6,7-tetrahydrobenzothiophen-6-amine hydrochloride (79)

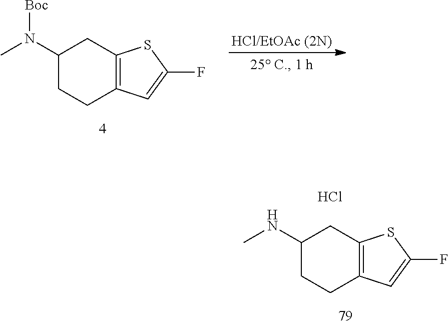 [SMILES: CN(C(=O)OC(C)(C)C)C1CCc2cc(F)sc2C1 ; "HCl/EtOAc (2N)" ; Cl ; CNC1CCc2cc(F)sc2C1]

To a solution of tert-butyl N-(2-fluoro-4,5,6,7-tetrahydrobenzothiophen-6-yl)-N-methyl-carbamate (4) (0.1 g, 350.41 μmol, 1 eq) in EtOAc (1 mL) was added HCl/EtOAc (1 mL). The mixture was stirred at 25° C. for 1 hr. The reaction mixture was filtered to give a cake. The cake was washed with ethyl acetate (3 mL) and dried under reduced pressure to give a white solid. Compound 2-fluoro-N-methyl-4,5,6,7-tetrahydrobenzothiophen-6-amine hydrochloride (79) (28.0 mg, 119.97 μmol, 34.24% yield). ¹H NMR (400 MHz, METHANOL-d₄) δ 6.27 (d, J=2.00 Hz, 1H), 3.59-3.50 (m, 1H), 3.15 (br dd, J=15.70, 5.19 Hz, 1H), 2.81-2.67 (m, 6H), 2.30-2.21 (m, 1H), 1.92 (dddd, J=13.01, 10.54, 9.10, 6.38 Hz, 1H). LCMS: m/z [M+H]⁺=186.2

### Procedure for preparation of tert-butyl N-(2-fluoro-4,5,6,7-tetrahydrobenzothiophen-6-yl)-N-methyl-carbamate enantiomer 1 (4a) and enantiomer 2 (4b)

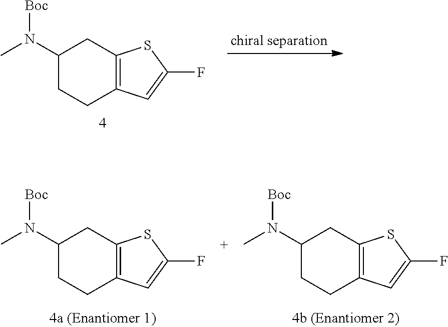

Racemic tert-butyl N-(2-fluoro-4,5,6,7-tetrahydrobenzothiophen-6-yl)-N-methyl-carbamate (4) (200 mg) was separated by SFC (column: DAICEL CHIRALPAK IG (250 mm*50 mm, 10 μm); mobile phase: [CO₂-EtOH (0.1% NH₃H₂O)]; B %: 10%-10%, 6 min), to give enantiomer 1 (4a, 77 mg, 1^{st} peak in SFC) as a yellow oil and enantiomer 2 (4b, 93 mg, 2^{nd} peak in SFC) as a yellow oil.

### Procedure for preparation of 2-fluoro-N-methyl-4,5,6,7-tetrahydrobenzothiophen-6-amine hydrochloride enantiomer 1 (80)

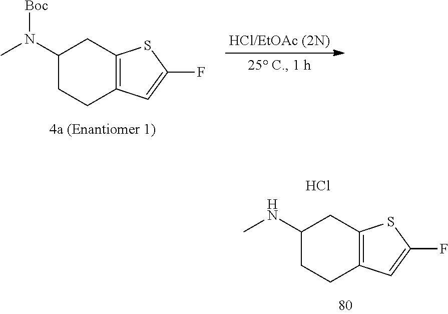 [SMILES: CN(C(=O)OC(C)(C)C)C1CCc2cc(F)sc2C1 ; Cl ; CNC1CCc2cc(F)sc2C1]

To a solution of tert-butyl N-(2-fluoro-4,5,6,7-tetrahydrobenzothiophen-6-yl)-N-methyl-carbamate enantiomer 1 (4a) (0.077 g, 269.82 μmol, 1 eq) in EtOAc (1 mL) was added 4N HCl/EtOAc (1 mL). The mixture was stirred at 25° C. for 1 hr. The reaction mixture was filtered to give a cake. The cake was washed with ethyl acetate (0.5 mL) and dried under reduced pressure to give a white solid. Compound 2-fluoro-N-methyl-4,5,6,7-tetrahydrobenzothiophen-6-amine hydrochloride enantiomer 1 (80) (44.89 mg, 192.34 μmol, 71.28% yield) was obtained as a white solid. ¹H NMR (400 MHz, METHANOL-d₄) δ 6.27 (d, J=2.00 Hz, 1H), 3.60-3.510 (m, 1H), 3.20-3.11 (m, 1H), 2.82-2.65 (m, 6H), 2.31-2.21 (m, 1H), 1.98-1.85 (m, 1H). LCMS: m/z [M+H]⁺=185.8

### Procedure for preparation of 2-fluoro-N-methyl-4,5,6,7-tetrahydrobenzothiophen-6-amine hydrochloride enantiomer 2 (81)

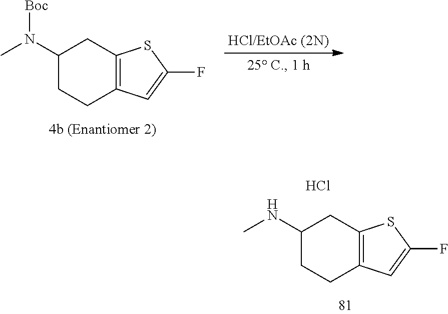 [SMILES: CN(C(=O)OC(C)(C)C)C1CCc2cc(F)sc2C1 ; Cl ; CNC1CCc2cc(F)sc2C1]

To a solution of tert-butyl N-(2-fluoro-4,5,6,7-tetrahydrobenzothiophen-6-yl)-N-methyl-carbamate enantiomer 2 (4b) (0.093 g, 325.89 μmol, 1 eq) in EtOAc (1 mL) was added HCl/EtOAc (1 mL). The mixture was stirred at 25° C. for 1 hr. The reaction mixture was filtered to give a cake. The cake was washed with ethyl acetate (0.5 mL) and dried under reduced pressure to give a white solid. Compound 2-fluoro-N-methyl-4,5,6,7-tetrahydrobenzothiophen-6-amine hydrochloride enantiomer 2 (81) (49.31 mg, 211.28 μmol, 64.83% yield) was obtained as a white solid. ¹H NMR (400 MHz, METHANOL-d₄) δ 6.27 (d, J=2.00 Hz, 1H), 3.55 (dddd, J=10.88, 8.25, 5.44, 2.81 Hz, 1H), 3.15 (br dd, J=15.70, 5.19 Hz, 1H), 2.86-2.62 (m, 6H), 2.34-2.20 (m, 1H), 1.92 (dddd, J=13.02, 10.49, 9.13, 6.38 Hz, 1H). LCMS: m/z [M+H]⁺=185.8

### Synthetic scheme for preparation of 2-fluoro-N-methyl-4,5,6,7-tetrahydrobenzothiophen-5-amine hydrochloride (82)

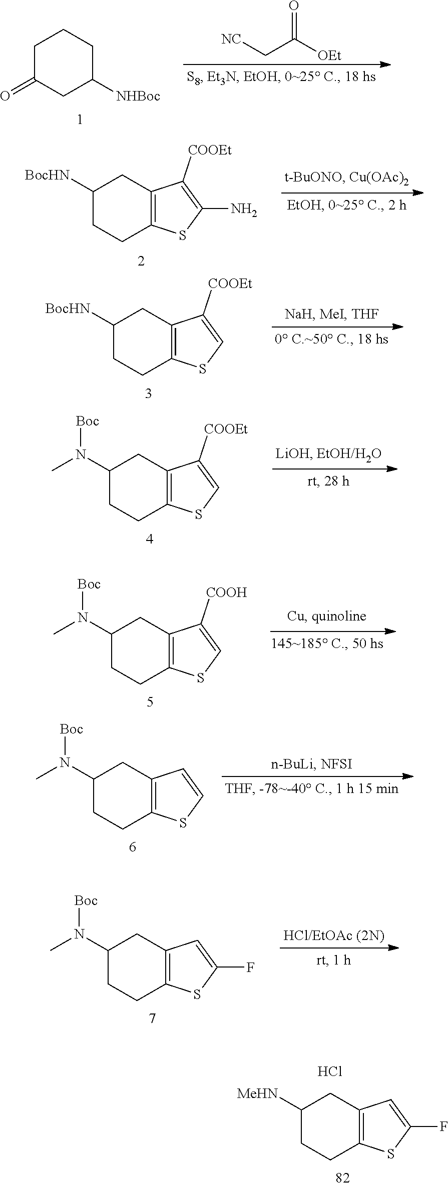

### Procedure for preparation of ethyl 2-amino-5-(tert-butoxycarbonylamino)-4,5,6,7-tetra hydrobenzothiophene-3-carboxylate (2)

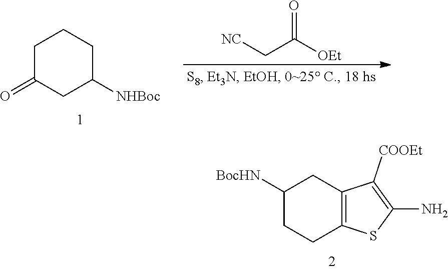 [SMILES: CC(C)(C)OC(=O)NC1CCCC(=O)C1 ; CCOC(=O)CC#N ; CCOC(=O)c1c(N)sc2c1CC(NC(=O)OC(C)(C)C)CC2]

To a solution of tert-butyl N-(3-oxocyclohexyl)carbamate (1) (50 g, 234.44 mmol, 1 eq) in EtOH (400 mL) was added S8 (12.03 g, 375.11 mmol, 1.6 eq) at 0° C., followed by ethyl 2-cyanoacetate (31.82 g, 281.33 mmol, 30.02 mL, 1.2 eq), the mixture was treated with TEA (47.45 g, 468.88 mmol, 65.26 mL, 2 eq) (maintaining internal temperature below 10° C.). The reaction was stirred at 25° C. for 18 hours. The reaction was filtered, the filtrate was concentrated to get a residue, to which was added PE/EtOAc (PE:EtOAc=3:1, 500 mL) and stirred at r.t. for 2 hours, the yellow solid was collected by filtration to obtain ethyl 2-amino-5-(tert-butoxycarbonylamino)-4,5,6,7-tetrahydrobenzothiophene-3-carboxylate (2) (80 g, crude).

### Procedure for preparation of ethyl 5-(tert-butoxycarbonylamino)-4,5,6,7-tetrahydro benzothiophene-3-carboxylate (3)

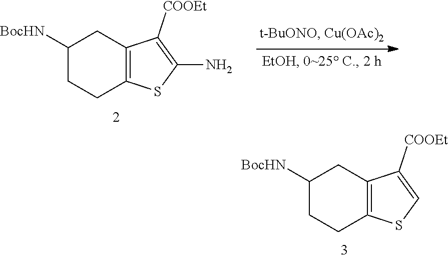 [SMILES: CCOC(=O)c1c(N)sc2c1CC(NC(=O)OC(C)(C)C)CC2 ; CCOC(=O)c1csc2c1CC(NC(=O)OC(C)(C)C)CC2]

To a solution of ethyl 2-amino-5-(tert-butoxycarbonylamino)-4,5,6,7-tetrahydrobenzothioph ene-3-carboxylate (2) (80 g, 234.99 mmol, 1 eq) in EtOH (800 mL) was added Cu(OAc)₂ (85.37 g, 469.98 mmol, 2 eq), then tert-butyl nitrite (24.23 g, 234.99 mmol, 27.95 mL, 1 eq) was added at 0° C. under N₂, the mixture was stirred at 0~25° C. for 2 hours. The reaction was quenched with sat. aq. NH₄Cl (500 mL), then concentrated to remove most of the MeOH, then diluted with EtOAc (1 L), the organic layer was separated and washed with brine (500 mL), dried over anhydrous Na₂SO₄, filtered and concentrated to get a residue. The residue was purified by silica gel column (PE:EtOAc=20:1 to 3:1) to yield ethyl 5-(tert-butoxycarbonylamino)-4,5,6,7-tetrahydrobenzothiophene-3-carboxylate (3) (50 g, 153.65 mmol, 65.38% yield) as yellow brown oil.

### Procedure for preparation of ethyl 5-[tert-butoxycarbonyl(methyl)amino]-4,5,6,7-tetrahydrobenzothiophene-3-carboxylate (4)

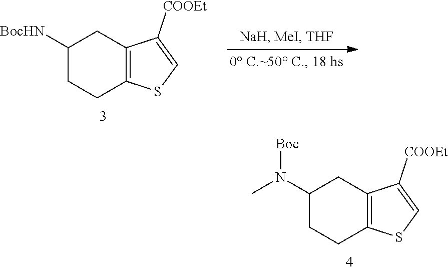 [SMILES: CCOC(=O)c1csc2c1CC(NC(=O)OC(C)(C)C)CC2 ; CCOC(=O)c1csc2c1CC(N(C)C(=O)OC(C)(C)C)CC2]

To a solution of ethyl 5-(tert-butoxycarbonylamino)-4,5,6,7-tetrahydrobenzothiophene-3-carboxylate (37.8 g, 116.16 mmol, 1 eq) in THF (400 mL) was added NaH (10.03 g, 250.90 mmol, 60% suspension in mineral oil, 2.16 eq) portionwise at 0° C., the mixture was stirred at 0~10° C. for 0.5 hour, then MeI (50.29 g, 354.28 mmol, 22.06 mL, 3.05 eq) was added, the mixture was stirred at 40-50° C. for 17.5 hours. The reaction was cooled to r.t, then quenched with sat. aq. NH₄Cl (200 mL) under ice bath, diluted with water (200 mL), the THF layer was separated and concentrated to get residue; the aqueous layer was extracted with EtOAc (200 mL), the organic layers were combined and washed with brine (200 mL), dried over anhydrous Na₂SO₄, filtered and concentrated to get a residue. The residue was purified by silica gel column (PE:EtOAc=20:1 to 10:1) to afford ethyl 5-[tert-butoxycarbonyl(methyl)amino]-4,5,6,7-tetrahydrobenzothiophene-3-carboxylate (4) (15 g, 44.19 mmol, 38.04% yield) as yellow oil.

### Procedure for preparation of 5-[tert-butoxycarbonyl(methyl)amino]-4,5,6,7-tetrahydro benzothiophene-3-carboxylic acid (5)

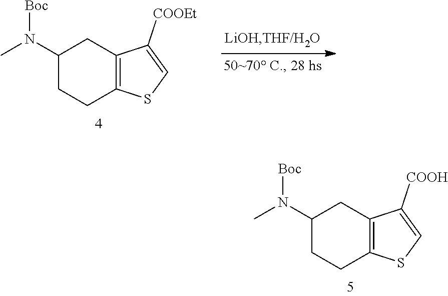 [SMILES: CCOC(=O)c1csc2c1CC(N(C)C(=O)OC(C)(C)C)CC2 ; CN(C(=O)OC(C)(C)C)C1CCc2scc(C(=O)O)c2C1]

To a solution of ethyl 5-[tert-butoxycarbonyl(methyl)amino]-4,5,6,7-tetrahydrobenzothio phene-3-carboxylate (4) (7.5 g, 22.09 mmol, 1 eq) in THF (80 mL) and H₂O (20 mL) was added LiOH·H₂O (6.03 g, 143.62 mmol, 6.5 eq), the mixture was stirred at 50~60° C. for 18 hours, additional LiOH·H₂O (5.10 g, 121.52 mmol, 5.5 eq) was added, the mixture was stirred at 50~60° C. for another 2 hours, then the mixture was stirred at 60~70° C. for another 8 hours. The reaction was cooled to rt, the THF layer was separated and concentrated to get a residue, which was dissolved in EtOAc (150 mL) and combined with the aqueous layer, then adjusted pH to 5~6 by 2N HCl, then extracted with EtOAc/MeOH (EtOAc:MeOH=10:1, 100 mL*3), the organic layers were combined and washed with brine (100 mL*2), dried over anhydrous Na₂SO₄, filtered and concentrated to afford 5-[tert-butoxycarbonyl(methyl)amino]-4,5,6,7-tetrahydrobenzothiophene-3-carboxylic acid (5) (6.8 g, 21.84 mmol, 98.83% yield) as yellow solid.

### Procedure for preparation of tert-butyl N-methyl-N-(4,5,6,7-tetrahydrobenzothiophen-5-yl)carbamate (6)

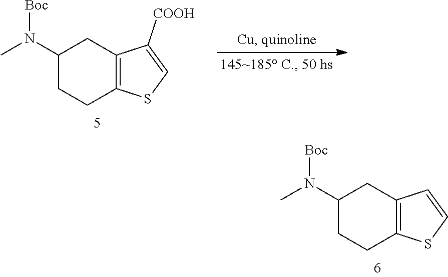 [SMILES: CN(C(=O)OC(C)(C)C)C1CCc2scc(C(=O)O)c2C1 ; CN(C(=O)OC(C)(C)C)C1CCc2sccc2C1]

To a solution of 5-[tert-butoxycarbonyl(methyl)amino]-4,5,6,7-tetrahydrobenzothiophene-3-carboxylic acid (5) (6.8 g, 21.84 mmol, 1 eq) in quinoline (50 mL) was added Cu (2.22 g, 34.94 mmol, 1.6 eq), the mixture was stirred at 185° C. for 3 hours and 145° C. for another 47 hours. The reaction was cooled to r.t., quenched with 2N HCl (150 mL*2), extracted with EA (100 mL*2), the organic layers were combined and washed with brine (100 mL), dried over anhydrous Na₂SO₄, filtered and then concentrated to get a residue. The residue was purified by silica gel column (PE:EtOAc=100:1 to 10:1) to afford tert-butyl N-methyl-N-(4,5,6,7-tetrahydrobenzothiophen-5-yl)carbamate (6) (3.5 g, 13.09 mmol, 59.94% yield) as yellow oil. ¹H NMR (400 MHz, CHLOROFORM-d) δ=7.08 (d, J=4.8 Hz, 1H), 6.74 (d, J=4.8 Hz, 1H), 4.61-4.20 (m, 1H), 3.00-2.89 (m, 2H), 2.81 (s, 3H), 2.73-2.62 (m, 2H), 2.00-1.92 (m, 2H), 1.48 (s, 9H)

### Procedure for preparation of tert-butyl N-(2-fluoro-4,5,6,7-tetrahydrobenzothiophen-5-yl)-N-methyl-carbamate (7)

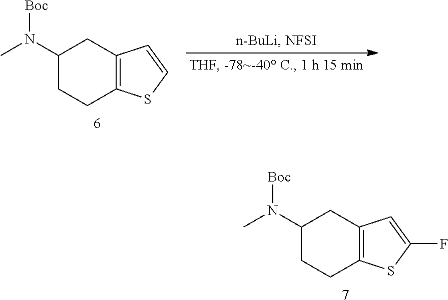

To a solution of tert-butyl N-methyl-N-(4,5,6,7-tetrahydrobenzothiophen-5-yl)carbamate (6) (2.5 g, 9.35 mmol, 1 eq) in THF (20 mL) was dropwise added n-BuLi (2.5 M, 5.05 mL, 1.35 eq) under N₂ at −78° C., the mixture was stirred at −78° C. for 15 min, then NFSI (2.95 g, 9.35 mmol, 1 eq) in THF (20 mL) was added dropwise, the mixture was stirred at −78~−40° C. for 1 hour. The reaction was quenched by sat. aq. NH₄Cl (40 mL) and 2 N HCl (10 mL), extracted by EtOAc (40 mL), the organic layer was separated and dried over anhydrous Na₂SO₄, filtered and then concentrated to get a residue. The residue was purified by reversed-phase HPLC with the following method: column: UniSil 10-120 C18 50×250 mm; mobile phase: [water (FA)-ACN]; B %: 52%-82%, 25 min. The product tert-butyl N-(2-fluoro-4,5,6,7-tetrahydrobenzothiophen-5-yl)-N-methyl-carbamate (7) (1.2 g, 3.78 mmol, 30.86% yield) was obtained as yellow oil.

### Procedure for preparation of 2-fluoro-N-methyl-4,5,6,7-tetrahydrobenzothiophen-5-amine hydrochloride (82)

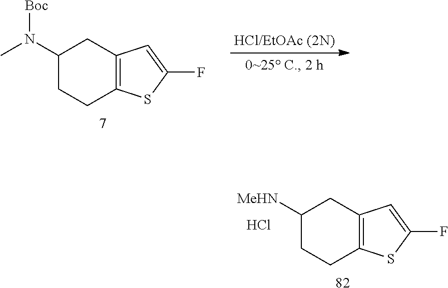

To a solution of tert-butyl N-(2-fluoro-4,5,6,7-tetrahydrobenzothiophen-5-yl)-N-methyl-carbamate (7) (200 mg, 700.83 μmol, 1 eq) in EtOAc (2 mL) was added HCl/EtOAc (4 M, 2 mL, 11.42 eq). The mixture was stirred at 0~25° C. for 2 hr. The reaction mixture was quenched by addition NaHCO₃ saturated solution 10 ml at 0° C., and then extracted with EtOAc 30 ml (10 mL*3). The combined organic layers were dried over Na₂SO₄, filtered and concentrated under reduced pressure to give a residue. The crude product was purified by prep-HPLC (column: Welch Xtimate C18 150*25 mm*5 μm; mobile phase: [water (HCl)-ACN]; B %: 3%-33%, 8 min) to afford 2-fluoro-N-methyl-4,5,6,7-tetrahydro benzothiophen-5-amine hydrochloride (82) (54.9 mg, 293.37 μmol, 41.86% yield) as a white solid. 1H NMR (400 MHz, METHANOL-d4) δ=6.28 (d, J=1.8 Hz, 1H), 3.54-3.47 (m, 1H), 3.05 (br dd, J=5.0, 16.0 Hz, 1H), 2.86-2.80 (m, 2H), 2.78 (s, 3H), 2.67-2.58 (m, 1H), 2.38-2.27 (m, 1H), 2.03-1.87 (m, 1H). LCMS: m/z [M+H]⁺=186.1.

### Synthetic scheme for preparation of 3-fluoro-N-methyl-4,5,6,7-tetrahydrobenzothiophen-6-amine hydrochloride enantiomer 1 (83) and enantiomer 2 (84)

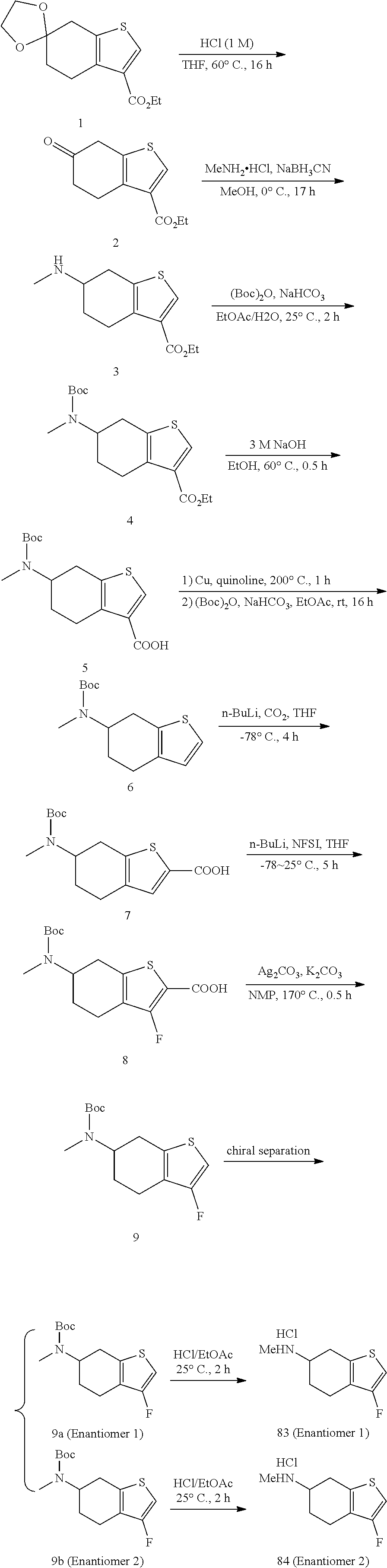

### Procedure for preparation of ethyl 6-oxo-5,7-dihydro-4H-benzothiophene-3-carboxylate (2)

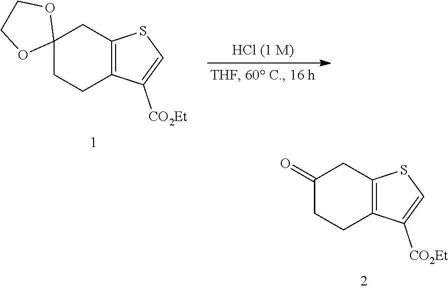 [SMILES: CCOC(=O)c1csc2c1CCC1(C2)OCCO1 ; CCOC(=O)c1csc2c1CCC(=O)C2]

To a solution of ethyl spiro[1,3-dioxolane-2,6′-5,7-dihydro-4H-benzothiophene]-3′-carboxylate (1) (22 g, 81.99 mmol, 1 eq) in THF (150 mL) was added HCl (1 M, 150 mL, 1.83 eq). The mixture was stirred at 60° C. for 16 hr. The mixture was then cooled and extracted with EtOAc (100 mL*3). The combined organic phase was washed with brine (100 mL*3), dried over Na₂SO₄, filtered and concentrated at reduced pressure to give a residue. The residue was purified by silica gel chromatography (100-200 mesh silica gel, Petroleum ether/Ethyl acetate=1/0~10/1, Rf=0.55) to afford ethyl 6-oxo-5,7-dihydro-4H-benzothiophene-3-carboxylate (2) (15.3 g, 68.22 mmol, 83.21% yield) as a white solid. LCMS: m/z [M+H]⁺=225.1.

### Procedure for preparation of ethyl 6-(methylamino)-4,5,6,7-tetrahydrobenzothiophene-3-carboxylate (3)

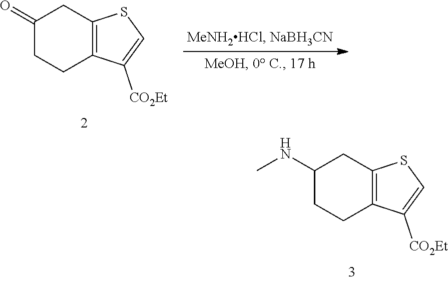 [SMILES: CCOC(=O)c1csc2c1CCC(=O)C2 ; CCOC(=O)c1csc2c1CCC(NC)C2]

To a solution of ethyl 6-oxo-5,7-dihydro-4H-benzothiophene-3-carboxylate (2) (15.3 g, 68.22 mmol, 1 eq) in MeOH (200 mL) was added methylamine hydrochloride (9.21 g, 136.44 mmol, 2 eq). The mixture was stirred at 0° C. for 1 h. Then to the mixture was added NaBH₃CN (12.86 g, 204.66 mmol, 3 eq) at 0° C. under N₂ and the mixture stirred at 25° C. for 16 h. The reaction mixture was then quenched with H₂O (200 mL). The mixture was extracted with EtOAc (200 mL*3). The combined organic phase was washed with brine (200 mL*3), dried over Na₂SO₄, filtered and concentrated at reduced pressure to give a residue. The crude product ethyl 6-(methylamino)-4,5,6,7-tetrahydrobenzothiophene-3-carboxylate (3) (16 g, crude) was obtained as a white solid and was used into the next step without further purification. LCMS: m/z [M+H]⁺=240.1.

### Procedure for preparation of ethyl 6-[tert-butoxycarbonyl(methyl)amino]-4,5,6,7-tetrahydrobenzothiophene-3-carboxylate (4)

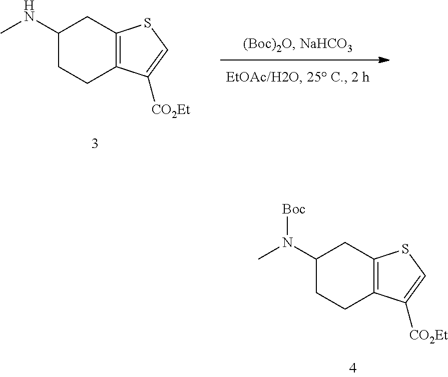 [SMILES: CCOC(=O)c1csc2c1CCC(NC)C2 ; CCOC(=O)c1csc2c1CCC(N(C)C(=O)OC(C)(C)C)C2]

To a solution of ethyl 6-(methylamino)-4,5,6,7-tetrahydrobenzothiophene-3-carboxylate (3) (16 g, 66.85 mmol, 1 eq) and (Boc)₂O (29.18 g, 133.70 mmol, 30.72 mL, 2 eq) in EtOAc (150 mL) and H₂O (150 mL) was added NaHCO₃ (28.08 g, 334.26 mmol, 13.01 mL, 5 eq). The mixture was stirred at 25° C. for 2 hr. The mixture was extracted with EtOAc (200 mL*3). The combined organic phase was washed with brine (200 mL*3), dried over Na₂SO₄, filtered and concentrated at reduced pressure to give a residue. The residue was purified by silica gel chromatography (100-200 mesh silica gel, Petroleum ether/Ethyl acetate=1/0~10/1, Rf=0.52) to afford ethyl 6-[tert-butoxycarbonyl(methyl)amino]-4,5,6,7-tetrahydrobenzothiophene-3-carboxylate (4) (12.7 g, 37.41 mmol, 55.96% yield) as a white solid. ¹H NMR (400 MHz, Methanol-d4, 298 K) 6=8.02 (s, 1H), 4.27 (q, J=7.1 Hz, 3H), 3.23 (td, J=3.2, 17.4 Hz, 1H), 2.97-2.74 (m, 6H), 1.98-1.85 (m, 2H), 1.48 (s, 9H), 1.35 (t, J=7.1 Hz, 3H)

### Procedure for preparation of 6-[tert-butoxycarbonyl(methyl)amino]-4,5,6,7-tetrahydrobenzothiophene-3-carboxylic acid (5)

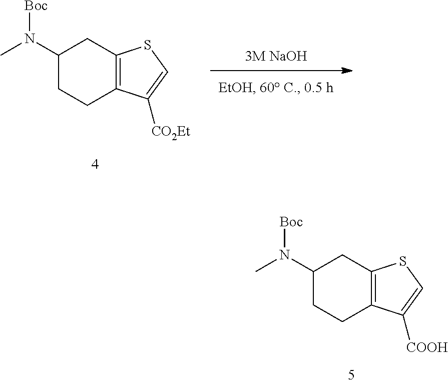 [SMILES: CCOC(=O)c1csc2c1CCC(N(C)C(=O)OC(C)(C)C)C2 ; "3M NaOH" ; CN(C(=O)OC(C)(C)C)C1CCc2c(C(=O)O)csc2C1]

To a solution of ethyl 6-[tert-butoxycarbonyl(methyl)amino]-4,5,6,7-tetrahydrobenzothiophene-3-carboxylate (4) (8.7 g, 25.63 mmol, 1 eq) in MeOH (20 mL) was added NaOH (3 M, 17.09 mL, 2 eq). The mixture was stirred at 60° C. for 0.5 hr. The mixture was concentrated at reduced pressure to give a residue and acidified to pH=7 by HCl (1 M) and then acidified to pH=5 by citric acid. The mixture was extracted with EtOAc (50 mL*3). The combined organic phase was washed with brine (10 mL*3), dried over Na₂SO₄, filtered and concentrated at reduced pressure. The crude product 6-[tert-butoxycarbonyl(methyl)amino]-4,5,6,7-tetrahydrobenzothiophene-3-carboxylic acid (5) (7.4 g, 23.76 mmol, 92.72% yield) was obtained as a white solid and used into the next step without further purification. LCMS: m/z [M+H-Boc]⁺=212.1.

### Procedure for preparation of tert-butyl N-methyl-N-(4,5,6,7-tetrahydrobenzothiophen-6-yl) carbamate (6)

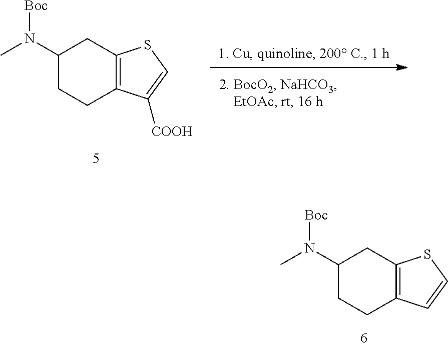 [SMILES: CN(C(=O)OC(C)(C)C)C1CCc2c(C(=O)O)csc2C1 ; "1. Cu, quinoline, 200° C., 1 h" ; CN(C(=O)OC(C)(C)C)C1CCc2ccsc2C1]

To a solution of 6-[tert-butoxycarbonyl(methyl)amino]-4,5,6,7-tetrahydrobenzothiophene-3-carboxylic acid (5) (7.2 g, 23.12 mmol, 1 eq) in quinoline (50 mL) was added Cu (2.94 g, 46.24 mmol 2 eq). The mixture was stirred at 200° C. for 1 h. Then the mixture was filtered and (Boc)₂O (10.09 g, 46.24 mmol, 10.62 mL, 2 eq) and aqueous NaHCO₃ (3.88 g, 46.24 mmol, 1.80 mL, 2 eq) was added. The mixture was stirred at 25° C. for 16 h. The reaction mixture was diluted with H₂O (50 mL) and citric acid to adjust pH=7 was added. The mixture was extracted with EtOAc (100 mL*3). The combined organic layers were washed with brine (30 mL), dried over anhydrous Na₂SO₄, filtered and concentrated under reduced pressure to give a residue. The residue was purified by silica gel chromatography (100-200 mesh silica gel, Petroleum ether/Ethyl acetate=1/0~511, Rf=0.68) to afford tert-butyl N-methyl-N-(4,5,6,7-tetrahydrobenzothiophen-6-yl)carbamate (6) (4.7 g, 17.58 mmol, 76.02% yield) as a white solid. LCMS: m/z [M+H-tBu]⁺=212.3. ¹H NMR (400 MHz, Methanol-d4, 298 K) 6=7.11 (d, J=5.1 Hz, 1H), 6.73 (d, J=5.1 Hz, 1H), 4.33 (br s, 1H), 2.94-2.77 (m, 6H), 2.76-2.62 (m, 1H), 2.03-1.81 (m, 2H), 1.47 (s, 9H).

### Procedure for preparation of 6-[tert-butoxycarbonyl(methyl)amino]-4,5,6,7-tetrahydrobenzothiophene-2-carboxylic acid (7)

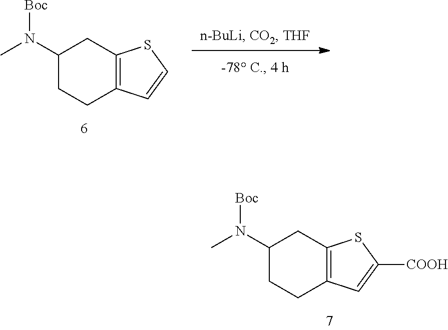

To a mixture of tert-butyl N-methyl-N-(4, 5, 6, 7-tetrahydrobenzothiophen-6-yl) carbamate (6) (4.2 g, 15.71 mmol, 1 eq) in THF (50 mL) was added n-BuLi (2.5 M, 6.91 mL, 1.1 eq) in one portion at −78° C. under N₂. The mixture was stirred at −78° C. for 0.5 hours. Then CO₂ (691.28 mg, 15.71 mmol, 1.5 μL, 1 eq) was bubbled into the mixture and purged with CO₂ (691.28 mg, 15.71 mmol, 1.5 μL, 1 eq) several times. The mixture was stirred under CO₂ (691.28 mg, 15.71 mmol, 1.5 μL, 1 eq) at −78° C. for 4 hours. The mixture was quenched with saturated ammonium chloride aqueous solution (20 mL). The mixture was extracted with EtOAc (20 mL*3). The combined organic phase was washed with brine (20 mL*3), dried over Na₂SO₄, filtered and concentrated at reduced pressure to give a residue. The residue was purified by silica gel chromatography (100-200 mesh silica gel, Petroleum ether/Ethyl acetate=1/1~0/1, Rf=0.27) to afford 6-[tert-butoxycarbonyl(methyl)amino]-4,5,6,7-tetrahydrobenzothiophene-2-carboxylic acid (7) (2.7 g, 8.67 mmol, 55.20% yield) as a white solid. LCMS: m/z [M+H-tBu]⁺=256.1. ¹H NMR (400 MHz, Methanol-d4) δ=7.43 (s, 1H), 4.33 (br s, 1H), 2.99-2.85 (m, 2H), 2.85-2.78 (m, 4H), 2.77-2.66 (m, 1H), 2.00-1.86 (m, 2H), 1.47 (s, 9H)

### Procedure for preparation of 6-[tert-butoxycarbonyl(methyl)amino]-3-fluoro-4,5,6,7-tetrahydrobenzothiophene-2-carboxylic acid (8)

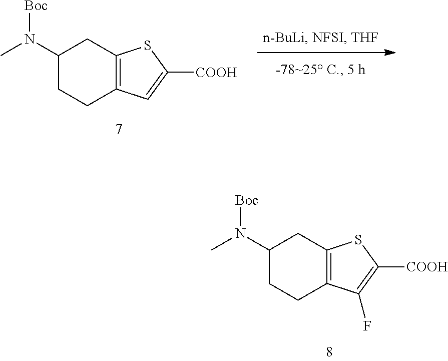

To a solution of 6-[tert-butoxycarbonyl(methyl)amino]-4,5,6,7-tetrahydrobenzothiophene-2-carboxylic acid (7) (2.7 g, 8.67 mmol, 1 eq) in THF (20 mL) was added n-BuLi (2.5 M, 7.63 mL, 2.2 eq) at −78° C. stirred for 0.5 h, then was added NFSI (3.01 g, 9.54 mmol, 1.1 eq) The mixture was stirred at −78° C. for 5 h. The mixture was then quenched with saturated ammonium chloride aqueous solution (50 mL). The mixture was extracted with EtOAc (50 mL*3). The combined organic phases were washed with brine (50 mL*3), dried over Na₂SO₄, filtered and concentrated at reduced pressure to give a residue. The residue was purified by silica gel chromatography (100-200 mesh silica gel, Petroleum ether/Ethyl acetate=1/1~0/1, Rf=0.26) to afford 6-[tert-butoxycarbonyl(methyl)amino]-3-fluoro-4,5,6,7-tetrahydrobenzothiophene-2-carboxylic acid (8) (1.9 g, 5.77 mmol, 66.53% yield) as a white solid. LCMS: m/z [M+H-tBu]⁺=274.1. ¹H NMR (400 MHz, Methanol-d4, 299 K) δ=4.42-4.21 (m, 1H), 2.98-2.87 (m, 1H), 2.83 (s, 3H), 2.76 (td, J=3.8, 16.6 Hz, 2H), 2.65-2.50 (m, 1H), 2.00-1.88 (m, 2H), 1.48 (s, 9H)

### Procedure for preparation of tert-butyl N-(3-fluoro-4,5,6,7-tetrahydrobenzothiophen-6-yl)-N-methyl-carbamate (9)

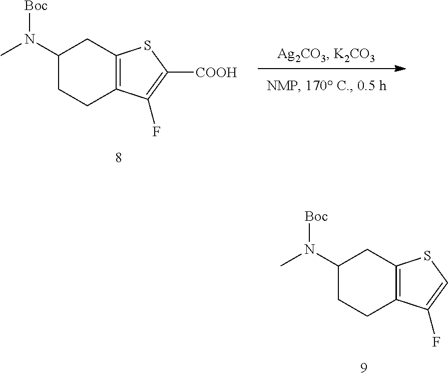

To a solution of 6-[tert-butoxycarbonyl(methyl)amino]-3-fluoro-4,5,6,7-tetrahydrobenzothiophene-2-carboxylic acid (8) (1.9 g, 5.77 mmol, 1 eq) in NMP (10 mL) was added K₂CO₃ (2.39 g, 17.30 mmol, 3 eq) and AgOAc (96.28 mg, 576.83 μmol, 29.54 L, 0.1 eq). The mixture was stirred at 170° C. for 0.5 hr. The mixture was extracted with EtOAc (50 mL*3). The combined organic phase was washed with brine (50 mL*3), dried over Na₂SO₄, filtered and concentrated at reduced pressure to give a residue. The residue was purified by silica gel chromatography (100-200 mesh silica gel, Petroleum ether/Ethyl acetate=1/1~0/1, Rf=0.34). The residue was further separated by prep-HPLC (column: Waters Xbridge Prep OBD C18 150*40 mm*10 μm; mobile phase: [water (NH₄HCO₃)-ACN]; B %: 50%-80%, 15 min) to afford tert-butyl N-(3-fluoro-4,5,6,7-tetrahydrobenzothiophen-6-yl)-N-methyl-carbamate (9) (0.9 g, 3.15 mmol, 54.67% yield) as a white solid. LCMS: m/z [M+H-tBu]⁺=230.1.

### Procedure for preparation of tert-butyl N-(3-fluoro-4,5,6,7-tetrahydrobenzothiophen-6-yl)-N-methyl-carbamate enantiomer 1 (9a) and enantiomer 2 (9b)

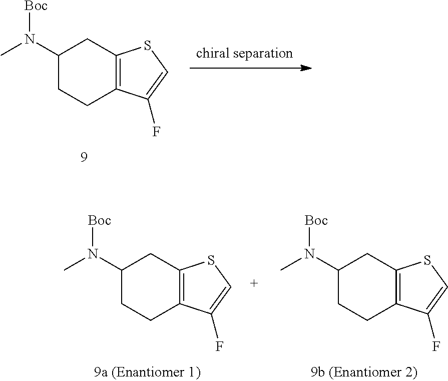 [SMILES: CN(C(=O)OC(C)(C)C)C1CCc2c(F)csc2C1 ; "chiral separation" ; CN(C(=O)OC(C)(C)C)C1CCc2c(F)csc2C1 ; CN(C(=O)OC(C)(C)C)C1CCc2c(F)csc2C1]

Racemic tert-butyl N-(3-fluoro-4,5,6,7-tetrahydrobenzothiophen-6-yl)-N-methyl-carbamate (9) was further separated by SFC (column: DAICEL CHIRALPAK AD (250 mm*30 mm, 10 μm); mobile phase: [CO₂-MeOH (0.1% NH₃H₂O)]; 15% B isocratic elution mode). Compound tert-butyl N-(3-fluoro-4,5,6,7-tetrahydrobenzothiophen-6-yl)-N-methyl-carbamate enantiomer 1 (9a) (200 mg, 700.83 μmol, 40.00% yield) was obtained as a white solid. LCMS: m/z [M+H-tBu]⁺=230.1. Compound tert-butyl N-(3-fluoro-4,5,6,7-tetrahydrobenzothiophen-6-yl)-N-methyl-carbamate enantiomer 2 (9b) (200 mg, 700.83 μmol, 40.00% yield) was obtained as a white solid. LCMS: m/z [M+H-tBu]⁺=230.1.

### Procedure for preparation of 3-fluoro-N-methyl-4,5,6,7-tetrahydrobenzothiophen-6-amine hydrochloride enantiomer 1 (83)

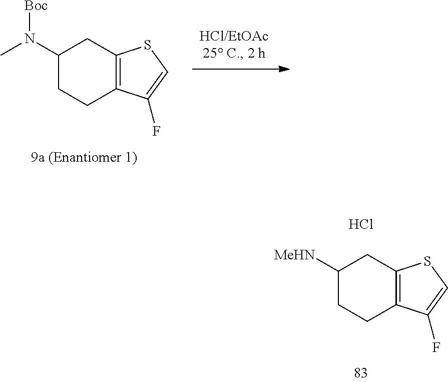 [SMILES: CN(C(=O)OC(C)(C)C)C1CCc2c(F)csc2C1 ; "HCl/EtOAc25° C., 2 h" ; Cl ; CNC1CCc2c(F)csc2C1]

To a solution of tert-butyl N-(3-fluoro-4,5,6,7-tetrahydrobenzothiophen-6-yl)-N-methyl-carbamate (9a) (200 mg, 700.83 μmol, 1 eq) in EtOAc (4 mL) was added HCl/EtOAc (2 mL). The mixture was stirred at 25° C. for 2 h. The reaction mixture was concentrated under reduced pressure to give a residue. The crude product was triturated with Ethyl acetate (EtOAc) at 25° C. for 30 min. Compound 3-fluoro-N-methyl-4, 5, 6, 7-tetrahydrobenzothiophen-6-amine hydrochloride enantiomer 1 (83) (122.31 mg, 551.64 μmol, 78.71% yield) was obtained as a white solid. LCMS: m/z [M+H]⁺=186.1. ¹H NMR (400 MHz, Methanol-d4, 298 K) 6=6.74 (s, 1H), 3.62-3.53 (m, 1H), 3.26 (br dd, J=5.2, 16.2 Hz, 1H), 2.86 (br dd, J=8.8, 16.1 Hz, 1H), 2.79 (s, 3H), 2.77-2.59 (m, 2H), 2.34-2.24 (m, 1H), 2.01-1.88 (m, 1H)

### Procedure for preparation of 3-fluoro-N-methyl-4,5,6,7-tetrahydrobenzothiophen-6-amine hydrochloride enantiomer 2 (84)

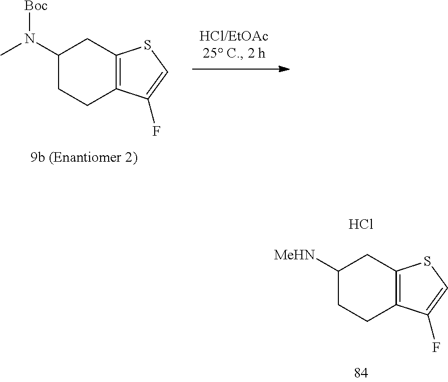 [SMILES: CN(C(=O)OC(C)(C)C)C1CCc2c(F)csc2C1 ; "HCl/EtOAc25° C., 2 h" ; Cl ; CNC1CCc2c(F)csc2C1]

To a solution of tert-butyl N-(3-fluoro-4, 5, 6, 7-tetrahydrobenzothiophen-6-yl)-N-methyl-carbamate (9b) (200 mg, 700.83 μmol, 1 eq) in EtOAc (4 mL) was added HCl/EtOAc (2 mL). The mixture was stirred at 25° C. for 2 h. The reaction mixture was concentrated under reduced pressure to give a residue. The crude product was triturated with Ethyl acetate at 25° C. for 30 min. Compound 3-fluoro-N-methyl-4,5,6,7-tetrahydrobenzothiophen-6-amine hydrochloride enantiomer 2 (84) (115.82 mg, 522.37 μmol, 74.54% yield) was obtained as a white solid. LCMS: m/z [M+H]⁺=186.1. ¹H NMR (400 MHz, Methanol-d4, 298 K) δ=6.74 (s, 1H), 3.57 (dddd, J=2.9, 5.4, 8.2, 10.8 Hz, 1H), 3.26 (br dd, J=5.1, 16.1 Hz, 1H), 2.86 (br dd, J=8.9, 15.8 Hz, 1H), 2.79 (s, 3H), 2.77-2.58 (m, 2H), 2.34-2.24 (m, 1H), 2.00-1.88 (m, 1H)

### Synthetic scheme for preparation of 5-chloro-N-methyl-3,4-dihydro-2H-thieno[3,4-b]pyran-3-amine hydrochloride (87)

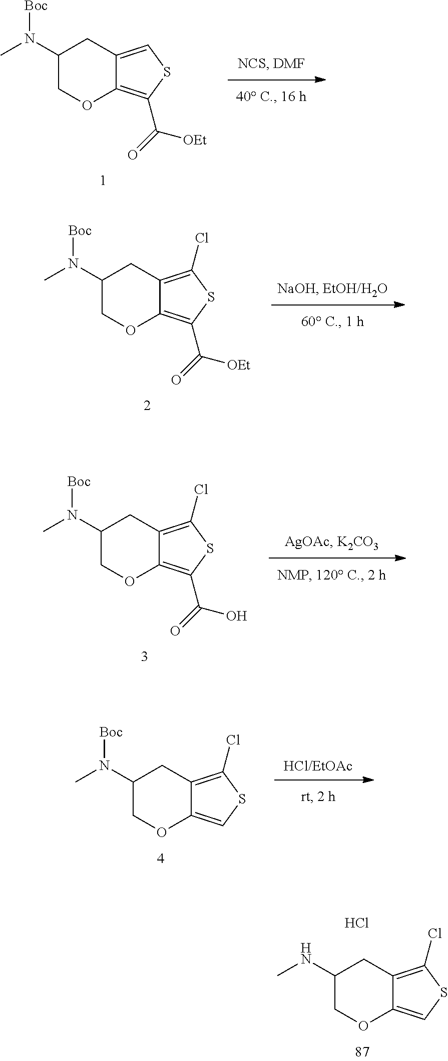

### Procedure for preparation of ethyl 3-[tert-butoxycarbonyl(methyl)amino]-5-chloro-3,4-dihydro-2H-thieno[3,4-b]pyran-7-carboxylate (2)

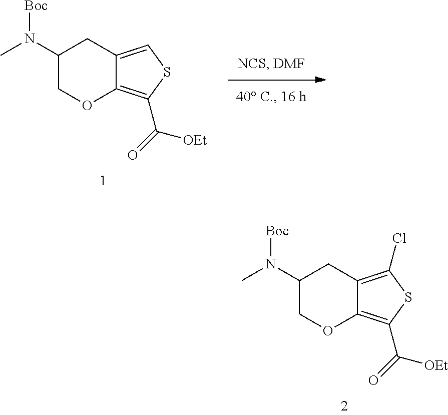 [SMILES: CCOC(=O)c1scc2c1OCC(N(C)C(=O)OC(C)(C)C)C2 ; CCOC(=O)c1sc(Cl)c2c1OCC(N(C)C(=O)OC(C)(C)C)C2]

To a solution of ethyl 3-[tert-butoxycarbonyl(methyl)amino]-3,4-dihydro-2H-thieno[3,4-b]pyran-7-carboxylate (1) (100 mg, 292.89 μmol, 1 eq) in DMF (1 mL) was added NCS (46.93 mg, 351.47 μmol, 1.2 eq). The mixture was stirred at 40° C. for 16 h. The mixture was quenched with H₂O (10 mL) and extracted with EtOAc (20 mL*2). The combined organic layers were washed with brine (10 mL), dried over anhydrous Na₂SO₄, filtered and concentrated under reduced pressure to give a residue. The residue was purified by silica gel chromatography (100-200 mesh silica gel, Petroleum ether/Ethyl acetate=1/0~3/1, Rf=0.57) to afford ethyl 3-[tert-butoxycarbonyl(methyl)amino]-5-chloro-3,4-dihydro-2H-thieno[3,4-b]pyran-7-carboxylate (2) (90 mg, 239.45 μmol, 81.75% yield) as a white gum. LCMS: m/z [M+H-tBu]⁺=320.1.

### Procedure for preparation of 3-[tert-butoxycarbonyl (methyl) amino]-5-chloro-3,4-dihydro-2H-thieno[3,4-b]pyran-7-carboxylic acid (3)

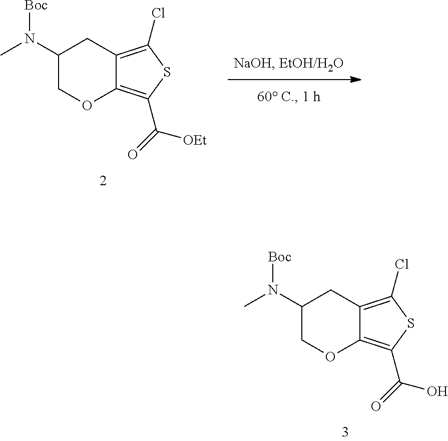 [SMILES: CCOC(=O)c1sc(Cl)c2c1OCC(N(C)C(=O)OC(C)(C)C)C2 ; CN(C(=O)OC(C)(C)C)C1COc2c(C(=O)O)sc(Cl)c2C1]

To a solution of ethyl 3-[tert-butoxycarbonyl(methyl)amino]-5-chloro-3,4-dihydro-2H-thieno[3,4-b]pyran-7-carboxylate (90 mg, 239.45 μmol, 1 eq) in EtOH (1 mL) was added NaOH (19.15 mg, 478.89 μmol, 159.63 μL, 2 eq) in H₂O (0.2 mL). The mixture was stirred at 60° C. for 1 hr. The mixture was concentrated at reduced pressure and acidified to pH=6 by HCl (1 M). The mixture was extracted with EtOAc (50 mL*3). The combined organic phases were washed with brine (10 mL*3), dried over Na₂SO₄, filtered and concentrated at reduced pressure to give a residue. The residue was purified by prep-TLC (SiO₂, Petroleum ether:Ethyl acetate=3:1, Rf=0.10) to afford 3-[tert-butoxycarbonyl(methyl)amino]-5-chloro-3,4-dihydro-2H-thieno[3,4-b]pyran-7-carboxylic acid (3) (70 mg, 201.26 μmol, 84.05% yield) as a white solid. LCMS: m/z [M+H-tBu]⁺=292.0.

### Procedure for preparation of tert-butyl N-(5-chloro-3,4-dihydro-2H-thieno[3,4-b]pyran-3-yl)-N-methyl-carbamate (4)

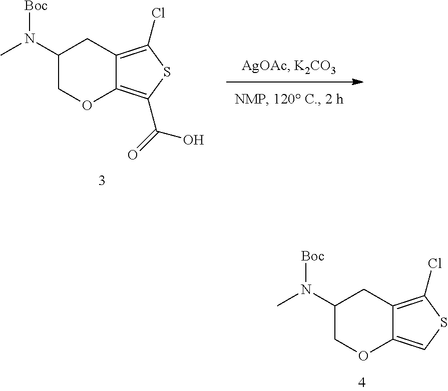

To a solution of 3-[tert-butoxycarbonyl(methyl)amino]-5-chloro-3,4-dihydro-2H-thieno[3, 4-b]pyran-7-carboxylic acid (3) (70 mg, 201.26 μmol, 1 eq) in NMP (1 mL) was added AgOAc (3.36 mg, 20.13 μmol, 1.03 μL, 0.1 eq) and K₂CO₃ (83.44 mg, 603.77 μmol, 3 eq). The mixture was stirred at 120° C. for 2 hr. The reaction mixture was filtered and concentrated under reduced pressure to give a residue. The residue was purified by prep-TLC (SiO₂, Petroleum ether:Ethyl acetate=3:1, Rf=0.65) to afford tert-butyl N-(5-chloro-3,4-dihydro-2H-thieno[3,4-b]pyran-3-yl)-N-methyl-carbamate (4) (50 mg, 164.58 μmol, 81.78% yield) as a white solid. LCMS: m/z [M+H-tBu]⁺=248.1.

### Procedure for preparation of 5-chloro-N-methyl-3,4-dihydro-2H-thieno[3,4-b]pyran-3-amine hydrochloride (87)

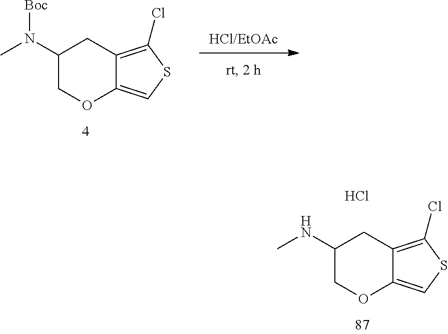 [SMILES: CN(C(=O)OC(C)(C)C)C1COc2csc(Cl)c2C1 ; "HCl/EtOAc" ; Cl ; CNC1COc2csc(Cl)c2C1]

To a solution of tert-butyl N-(5-chloro-3,4-dihydro-2H-thieno[3,4-b]pyran-3-yl)-N-methyl-carbamate (4) (50 mg, 164.58 μmol, 1 eq) in EtOAc (2 mL) was added HCl/EtOAc (1 mL). The mixture was stirred at 25° C. for 2 hr. The reaction mixture was concentrated under reduced pressure to give a residue. The crude product was purified by re-crystallization from EtOAc (10 mL) at 18° C. Compound 5-chloro-N-methyl-3,4-dihydro-2H-thieno[3,4-b]pyran-3-amine hydrochloride (87) (28 mg, 116.59 μmol, 70.84% yield) was obtained as a white solid. LCMS: m/z [M+H]⁺=204.0. ¹H NMR (400 MHz, Methanol-d4, 296 K) 6=6.49 (s, 1H), 4.52-4.43 (m, 1H), 4.17 (dd, J=1.4, 12.6 Hz, 1H), 3.76 (br d, J=1.6 Hz, 1H), 3.19-3.09 (m, 1H), 3.05-2.95 (m, 1H), 2.81 (s, 3H)

### Synthetic scheme for preparation of N, 5-dimethyl-3,4-dihydro-2H-thieno[3,4-b]pyran-3-amine hydrochloride (88)

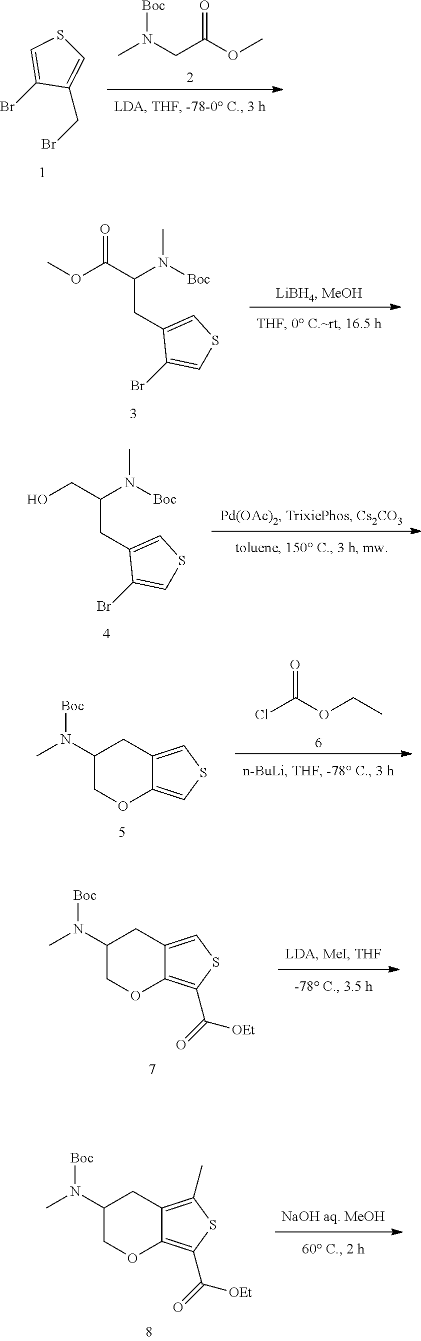

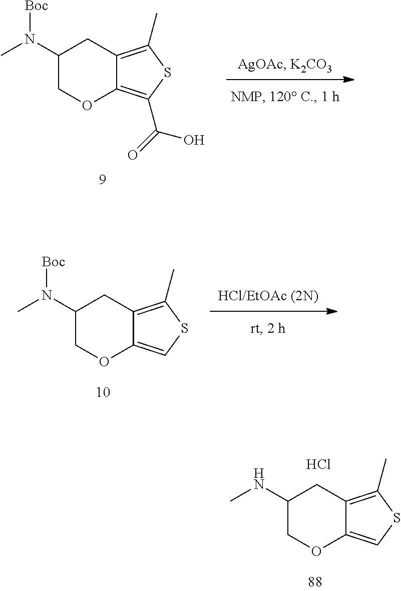

### Procedure for preparation of methyl 3-(4-bromothiophen-3-yl)-2-((tert-butoxycarbonyl)(methyl)amino)propanoate (3)

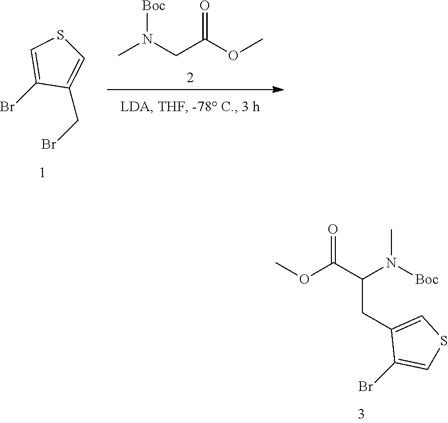

To a solution of LDA (2 M, 64.46 mL, 1.1 eq) in THF (200 mL) was added a solution of methyl 2-[tert-butoxycarbonyl (methyl) amino]acetate (22.87 g, 112.52 mmol, 0.96 eq) in THF (50 mL) at −78° C. for 0.5 h, then was added a solution of 3-bromo-4-(bromomethyl)thiophene (1) (30 g, 117.21 mmol, 1 eq) in THF (50 mL). The mixture was stirred at −78° C. for 2 hr under N₂. Then the mixture was warmed to 0° C. stirred for 1 hr under N₂. The mixture was quenched with saturated ammonium chloride aqueous solution (200 mL). The mixture was extracted with EtOAc (200 mL*3). The combined organic phases were washed with brine (100 mL*3), dried over Na₂SO₄, filtered and concentrated at reduced pressure to give a residue. The residue was purified by silica gel chromatography (100-200 mesh silica gel, Petroleum ether/Ethyl acetate=1/0~3/1, Rf=0.34) to afford methyl 3-(4-bromothiophen-3-yl)-2-((tert-butoxycarbonyl)(methyl)amino)propanoate (3) (18 g, 47.58 mmol, 40.60% yield) as a white solid. LCMS: m/z [M+H-Boc]⁺=280.0.

### Procedure for preparation of tert-butyl N-[1-[(4-bromo-3-thienyl) methyl]-2-hydroxy-ethyl]-N-methyl-carbamate (4)

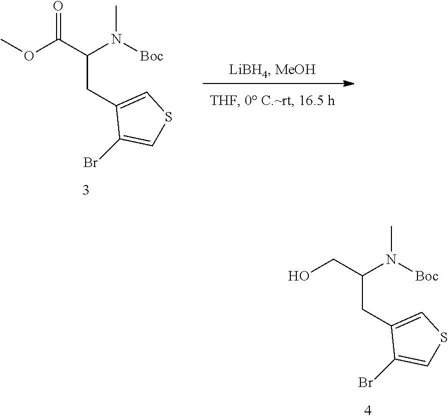

To a mixture of methyl 3-(4-bromothiophen-3-yl)-2-((tert-butoxycarbonyl)(methyl)amino)propanoate (3) (21 g, 55.51 mmol, 1 eq) in THF (200 mL) was added LiBH₄ (2 M, 33 mL, 1.19 eq) in one portion at 0° C. under N₂. The mixture was stirred at 0° C. for 30 min, then to the mixture was added MeOH (20 mL) and warmed to 25° C. and stirred for 16 hours. The mixture was quenched with aq. sat. NH₄Cl (200 mL). The mixture was extracted with EtOAc (200 mL*3). The combined organic phases were washed with brine (100 mL*3), dried over Na₂SO₄, filtered and concentrated at reduced pressure to give a residue. The residue was purified by silica gel chromatography (100-200 mesh silica gel, Petroleum ether/Ethyl acetate=1/1, Rf=0.37) to afford tert-butyl N-[1-[(4-bromo-3-thienyl) methyl]-2-hydroxy-ethyl]-N-methyl-carbamate (4) (17.1 g, 48.82 mmol, 87.94% yield) as a yellow oil. LCMS: m/z [M+H-tBu]⁺=294.0. ¹H NMR (400 MHz, Methanol-d4, 297 K) δ 7.43-7.36 (m, 1H), 7.19-7.13 (m, 1H), 4.49-4.36 (m, 1H), 3.70-3.59 (m, 2H), 2.79 (s, 3H), 2.73 (d, J=2.3 Hz, 2H), 1.39-1.27 (m, 9H)

### Procedure for preparation of tert-butyl (3,4-dihydro-2H-thieno[3,4-b]pyran-3-yl)(methyl)carbamate (5)

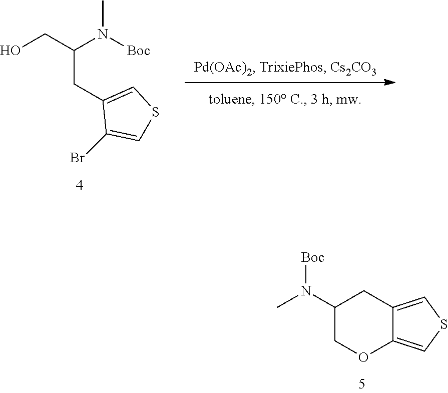 [SMILES: CN(C(=O)OC(C)(C)C)C(CO)Cc1cscc1Br ; "Pd(OAc)2, TrixiePhos, Cs2CO3" ; CN(C(=O)OC(C)(C)C)C1COc2cscc2C1]

Tert-butyl N-[1-[(4-bromo-3-thienyl) methyl]-2-hydroxy-ethyl]-N-methyl-carbamate (4) (1.5 g, 4.28 mmol, 1 eq), Cs₂CO₃ (2.09 g, 6.42 mmol, 1.5 eq), di-tert-butyl-[1-(1-naphthyl)-2-naphthyl]phosphane (85.33 mg, 214.12 μmol, 0.05 eq) and Pd(OAc)₂ (48.07 mg, 214.12 μmol, 0.05 eq) were taken up into a microwave tube in toluene (10 mL). The sealed tube was heated at 150° C. for 3 h under microwave. The reaction mixture was filtered and concentrated under reduced pressure to give a residue. The residue was purified by silica gel chromatography (100-200 mesh silica gel, Petroleum ether/Ethyl acetate=1/0~3/1, Rf=0.64) to afford tert-butyl (3,4-dihydro-2H-thieno[3,4-b]pyran-3-yl)(methyl)carbamate (5) (2.3 g, 8.54 mmol, 28.48% yield) as a white solid. LCMS: m/z [M+H-tBu]⁺=214.1.

### Procedure for preparation of ethyl 3-[tert-butoxycarbonyl (methyl)amino]-3,4-dihydro-2H-thieno[3, 4-b]pyran-7-carboxylate (7)

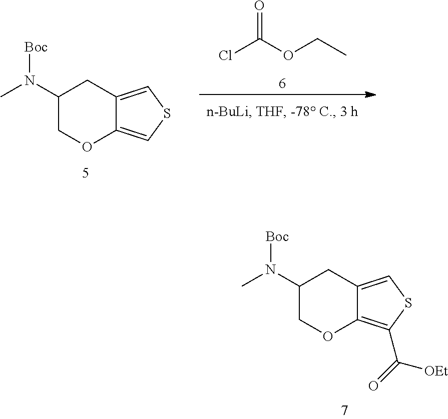

To a mixture of tert-butyl (3,4-dihydro-2H-thieno[3,4-b]pyran-3-yl)(methyl)carbamate (5) (500 mg, 1.86 mmol, 1 eq) in THF (6 mL) was added n-BuLi (2.5 M, 891.00 μL, 1.2 eq) in one portion at −78° C. under N₂. The mixture was stirred at −78° C. for 0.5 hour. Then ethyl carbonochloridate (420 mg, 3.87 mmol, 370.04 μL, 2.08 eq) was added. The mixture was stirred at −78° C. for 3 hours. The mixture was quenched with aq. sat. NH₄Cl (50 mL) and extracted with EtOAc (50 mL*3). The combined organic phase was washed with brine (50 mL*3), dried over Na₂SO₄, filtered and concentrated at reduced pressure to give a residue. The residue was purified by silica gel chromatography (100-200 mesh silica gel, Petroleum ether/Ethyl acetate=1/0~3/1, Rf=0.18) to afford ethyl 3-((tert-butoxycarbonyl)(methyl)amino)-3,4-dihydro-2H-thieno[3,4-b]pyran-7-carboxylate (7) (480 mg, 1.41 mmol, 75.74% yield) as a white solid. ¹H NMR (400 MHz, Methanol-d4, 296 K) δ (ppm)=7.33 (s, 1H), 4.38 (br d, J=1.1 Hz, 1H), 4.30-4.21 (m, 4H), 3.06-2.92 (m, 2H), 2.81 (s, 3H), 1.48 (s, 9H), 1.32 (t, J=7.1 Hz, 3H)

### Procedure for preparation of ethyl 3-((tert-butoxycarbonyl)(methyl)amino)-5-methyl-3,4-dihydro-2H-thieno[3,4-b]pyran-7-carboxylate (8)

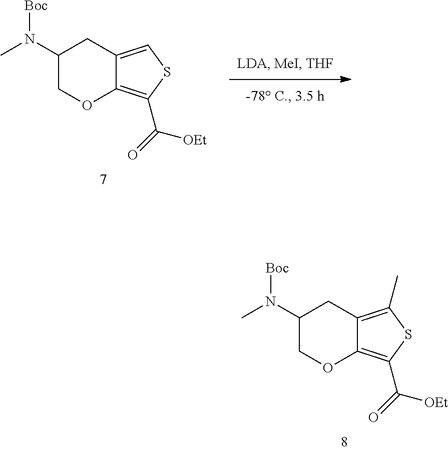 [SMILES: CCOC(=O)c1scc2c1OCC(N(C)C(=O)OC(C)(C)C)C2 ; "LDA, MeI, THF" ; CCOC(=O)c1sc(C)c2c1OCC(N(C)C(=O)OC(C)(C)C)C2]

To a mixture of ethyl 3-((tert-butoxycarbonyl)(methyl)amino)-3,4-dihydro-2H-thieno[3,4-b]pyran-7-carboxylate (7) (200 mg, 585.79 μmol, 1 eq) in THF (6 mL) was added LDA (2 M, 292.89 μL, 1 eq) in one portion at −78° C. under N₂. The mixture was stirred at −78° C. for 0.5 hours. Then MeI (108.09 mg, 761.52 μmol, 47.41 μL, 1.3 eq) was added. The mixture was stirred at −78° C. for 3 hours. The mixture was quenched with H₂O (10 mL). The mixture was extracted with EtOAc (20 mL*2). The combined organic layers were washed with brine (10 mL), dried over anhydrous Na₂SO₄, filtered and concentrated under reduced pressure to give a residue. The residue was purified by silica gel chromatography (100-200 mesh silica gel, Petroleum ether/Ethyl acetate=1/0~3/1, Rf=0.59) to afford ethyl 3-((tert-butoxycarbonyl)(methyl)amino)-5-methyl-3,4-dihydro-2H-thieno[3,4-b]pyran-7-carboxylate (8) (150 mg, 422.00 μmol, 72.04% yield) as a white solid. LCMS: m/z [M+H-tBu]⁺=300.2.

### Procedure for preparation of ethyl 3-((tert-butoxycarbonyl)(methyl)amino)-5-methyl-3,4-dihydro-2H-thieno[3,4-b]pyran-7-carboxylate (9)

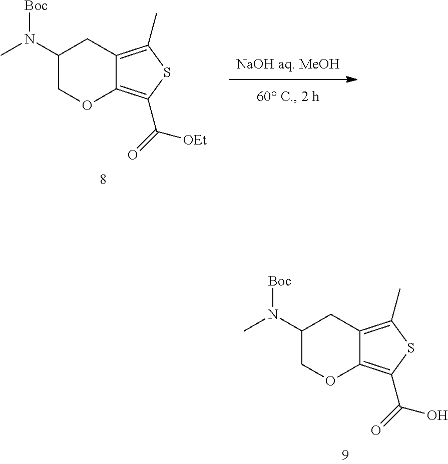 [SMILES: CCOC(=O)c1sc(C)c2c1OCC(N(C)C(=O)OC(C)(C)C)C2 ; "NaOH aq. MeOH" ; Cc1sc(C(=O)O)c2c1CC(N(C)C(=O)OC(C)(C)C)CO2]

To a solution of ethyl 3-((tert-butoxycarbonyl)(methyl)amino)-5-methyl-3,4-dihydro-2H-thieno[3,4-b]pyran-7-carboxylate (8) (150 mg, 422.00 μmol, 1 eq) in MeOH (3 mL) was added NaOH (3 M, 281.34 μL, 2 eq). The mixture was stirred at 60° C. for 2 hr. The mixture was concentrated at reduced pressure to give a residue, diluted with water and acidified to pH=6 by adding HCl (aq. 1 M). The mixture was extracted with EtOAc (50 mL*3). The combined organic phase was washed with brine (10 mL*3), dried over Na₂SO₄, filtered and concentrated at reduced pressure. The crude product 3-((tert-butoxycarbonyl)(methyl)amino)-5-methyl-3,4-dihydro-2H-thieno[3,4-b]pyran-7-carboxylic acid (9) (130 mg, 397.07 μmol, 94.09% yield) was obtained as a white solid and used into the next step without further purification. LCMS: m/z [M+H-tBu]⁺=272.1.

### Procedure for preparation of tert-butyl N-methyl-N-(5-methyl-3, 4-dihydro-2H-thieno[3, 4-b]pyran-3-yl) carbamate (10)

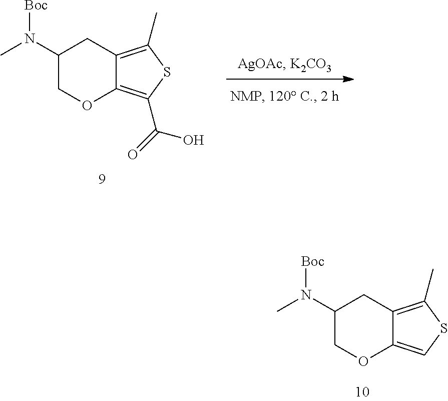

To a solution of 3-((tert-butoxycarbonyl)(methyl)amino)-5-methyl-3,4-dihydro-2H-thieno[3,4-b]pyran-7-carboxylic acid (9) (110 mg, 335.99 μmol, 1 eq) in NMP (1 mL) was added AgOAc (5.61 mg, 33.60 μmol, 1.72 μL, 0.1 eq) and K₂CO₃ (139.31 mg, 1.01 mmol, 3 eq). The mixture was stirred at 120° C. for 2 hr. The reaction mixture was filtered and concentrated under reduced pressure to give a residue. The residue was purified by silica gel chromatography (100-200 mesh silica gel, Petroleum ether/Ethyl acetate=1/0~511, Rf=0.44) to afford tert-butyl methyl(5-methyl-3,4-dihydro-2H-thieno[3,4-b]pyran-3-yl)carbamate (10) (95 mg, 335.23 μmol, 99.78% yield) as a white solid. LCMS: m/z [M+H-tBu]⁺=228.1.

### Procedure for preparation of N, 5-dimethyl-3,4-dihydro-2H-thieno[3,4-b]pyran-3-amine hydrochloride (88)

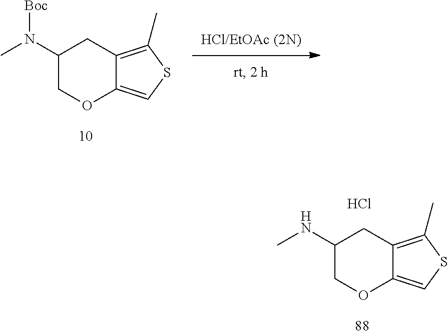 [SMILES: Cc1scc2c1CC(N(C)C(=O)OC(C)(C)C)CO2 ; "HCl/EtOAc (2N)" ; CNC1COc2csc(C)c2C1 ; Cl]

To a solution of tert-butyl methyl(5-methyl-3,4-dihydro-2H-thieno[3,4-b]pyran-3-yl)carbamate (10) (90 mg, 317.59 μmol, 1 eq) in EtOAc (1 mL) was added HCl/EtOAc (1 mL). The mixture was stirred at 25° C. for 2 hr. The reaction mixture was concentrated under reduced pressure to give a residue. The residue was purified by prep-HPLC (column: Welch Xtimate C18 150*25 mm*5 μm; mobile phase: [water (HCl)-ACN]; gradient: 0%-20% B over 9 min) to afford N, 5-dimethyl-3,4-dihydro-2H-thieno[3,4-b]pyran-3-amine hydrochloride (88) (34.77 mg, 158.24 μmol, 49.83% yieldl) as an orange solid. LCMS: m/z [M+H]⁺=184.3, ¹H NMR (400 MHz, Methanol-d4, 298 K) δ (ppm)=6.30 (s, 1H), 4.46-4.37 (m, 1H), 4.13 (d, J=12.5 Hz, 1H), 3.72 (br s, 1H), 3.15-3.06 (m, 1H), 3.02-2.93 (m, 1H), 2.80 (s, 3H), 2.34 (s, 3H).

### Synthetic Scheme for the preparation of 3-chloro-4,5,6,7-tetrahydro-2-benzothiophen-5-amine hydrochloride (89)

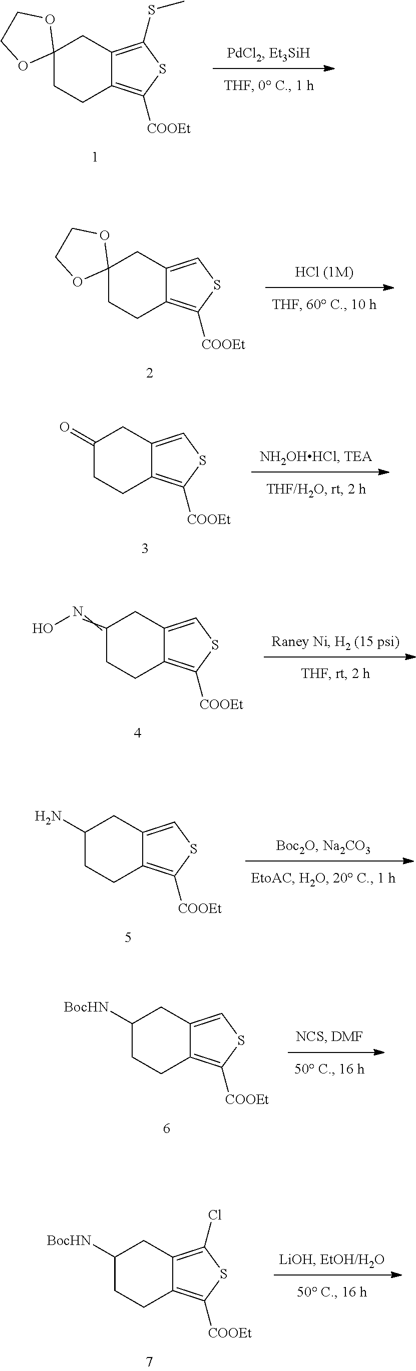

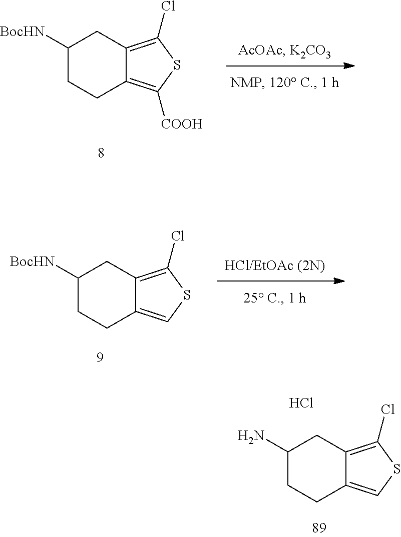

### Procedure for preparation of ethyl spiro[1,3-dioxolane-2,5′-6,7-dihydro-4H-2-benzothiophene]-1′-carboxylate (2)

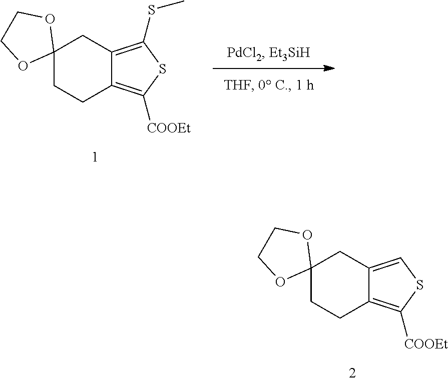

To a solution of ethyl 3′-methylsulfanylspiro[1,3-dioxolane-2,5′-6,7-dihydro-4H-2-benzothiophene]-1′-carboxylate (1) (20.13 g, 64.01 mmol, 1 eq) in THF (200 mL) was added PdCl₂ (908.06 mg, 5.12 mmol, 0.08 eq) and Et₃SiH (11.16 g, 96.02 mmol, 15.34 mL, 1.5 eq). The mixture was stirred at 0° C. for 1 hr. The mixture was filtered, then the filtrate was diluted with water and extracted with EtOAc 60 mL (30 mL*2). The combined organic layers were washed with aq. sat. NaCl 30 mL (15 mL*2), dried over Na₂SO₄, filtered and concentrated under reduced pressure to give a residue. The residue was purified by flash silica gel chromatography (ISCO®; 80 g SepaFlash® Silica Flash Column, eluent 0-10% Ethylacetate/Petroleum ether gradient @80 mL/min). Ethyl spiro[1,3-dioxolane-2,5′-6,7-dihydro-4H-2-benzothiophene]-1′-carboxylate (2) (14.7 g, 54.78 mmol, 85.58% yield) was obtained as yellow oil. ¹H NMR (400 MHz, CHLOROFORM-d) δ=7.09 (s, 1H), 4.32 (d, J=7.1 Hz, 2H), 4.03 (s, 4H), 3.23 (t, J=6.9 Hz, 2H), 2.92 (s, 2H), 1.96 (t, J=6.8 Hz, 2H), 1.36 (t, J=7.1 Hz, 3H)

### Procedure for preparation of ethyl 5-oxo-6,7-dihydro-4H-2-benzothiophene-1-carboxylate (3)

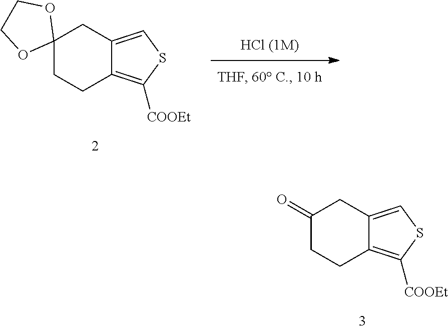 [SMILES: CCOC(=O)c1scc2c1CCC1(C2)OCCO1 ; "HCl (1M)" ; CCOC(=O)c1scc2c1CCC(=O)C2]

To a solution of ethyl spiro[1,3-dioxolane-2,5′-6,7-dihydro-4H-2-benzothiophene]-1′-carboxylate (2) (14.7 g, 54.78 mmol, 1 eq) in THF (150 mL) was added HCl (1 M, 150 mL, 2.74 eq). The mixture was stirred at 60° C. for 10 hr. The reaction mixture was then cooled and quenched by addition of aq. sat. NaHCO₃ (120 mL) and extracted with EtOAc (30 mL*3). The combined organic layers were washed with aq. sat. NaCl (30 mL*2), dried over Na₂SO₄, filtered and concentrated under reduced pressure to give a residue. The crude product ethyl 5-oxo-6,7-dihydro-4H-2-benzothiophene-1-carboxylate (3) (12.1 g, 53.95 mmol, 98.48% yield) was obtained as brown solid. ¹H NMR (400 MHz, CHLOROFORM-d) δ=7.18 (s, 1H), 4.36 (q, J=7.1 Hz, 2H), 3.58 (d, J=0.9 Hz, 2H), 3.46 (t, J=6.9 Hz, 2H), 2.64-2.55 (m, 2H), 1.39 (t, J=7.1 Hz, 3H)

### Procedure for preparation of ethyl 5-hydroxyimino-6,7-dihydro-4H-2-benzothiophene-1-carboxylate (4)

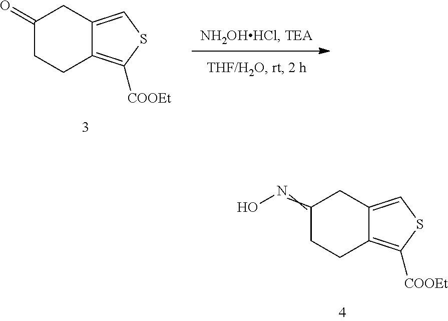 [SMILES: CCOC(=O)c1scc2c1CCC(=O)C2 ; "NH2OH·HCl, TEA" ; CCOC(=O)c1scc2c1CCC(=NO)C2]

To a solution of ethyl 5-oxo-6,7-dihydro-4H-2-benzothiophene-1-carboxylate (3) (1 g, 4.46 mmol, 1 eq) in THF (8 mL) and H₂O (4 mL) was added TEA (451.18 mg, 4.46 mmol, 620.61 μL, 1 eq) and NH₂OH·HCl (371.81 mg, 5.35 mmol, 1.2 eq). The mixture was stirred at 25° C. for 2 hr. The reaction mixture was extracted with ethyl acetate (20 mL*3). The combined organic layers were dried over Na₂SO₄, filtered and concentrated under reduced pressure to give a residue. The residue was purified by column chromatography (SiO₂, Petroleum ether/Ethyl acetate=10/1). Compound ethyl 5-hydroxyimino-6,7-dihydro-4H-2-benzothiophene-1-carboxylate (4) (0.856 g, 3.54 mmol, 79.43% yield) was obtained as a yellow solid. ¹H NMR (400 MHz, METHANOL-d₄) δ 7.40-7.35 (m, 1H), 4.35-4.25 (m, 2H), 3.77 (s, 1H), 3.50 (s, 1H), 3.26-3.16 (m, 2H), 2.76-2.69 (m, 1H), 2.51 (t, J=6.60 Hz, 1H) 1.39-1.26 (m, 3H)

### Procedure for preparation of ethyl 5-amino-4,5,6,7-tetrahydro-2-benzothiophene-1-carboxylate (5)

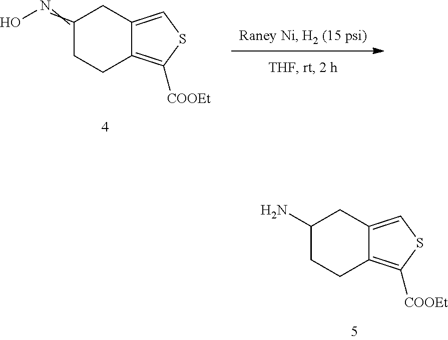 [SMILES: CCOC(=O)c1scc2c1CCC(=NO)C2 ; "Raney Ni, H2 (15 psi)" ; CCOC(=O)c1scc2c1CCC(N)C2]

To a solution of ethyl 5-hydroxyimino-6,7-dihydro-4H-2-benzothiophene-1-carboxylate (4) (0.45 g, 1.88 mmol, 1 eq) in THF (5 mL) was added Raney nickel (16.11 mg, 188.06 μmol, 0.1 eq) under N₂ atmosphere. The suspension was degassed and purged with H₂ for 3 times. The mixture was stirred under H₂ (15 Psi) at 25° C. for 2 hr. The reaction mixture was filtered and concentrated under reduced pressure. The crude product ethyl 5-amino-4,5,6,7-tetrahydro-2-benzothiophene-1-carboxylate (5) (0.29 g) was obtained as a yellow oil and was used into the next step without further purification.

### Procedure for preparation of ethyl 5-(tert-butoxycarbonylamino)-4,5,6,7-tetrahydro-2-benzothiophene-1-carboxylate (6)

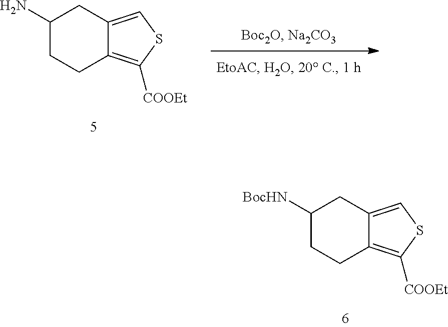

To a solution of ethyl 5-amino-4,5,6,7-tetrahydro-2-benzothiophene-1-carboxylate (5) (0.284 g, 1.26 mmol, 1 eq) in EtOAc (1 mL) and H₂O (1 mL) was added Na₂CO₃ (209.24 mg, 2.52 mmol, 2 eq) and (Boc)₂O (550.20 mg, 2.52 mmol, 579.16 L, 2 eq). The mixture was stirred at 20° C. for 1 hr. The reaction mixture was then diluted with H₂O 5 mL and extracted with EtOAc (5 mL*3). The combined organic layers were washed with aq. sat. NaCl (5 mL*3), dried over Na₂SO₄, filtered and concentrated under reduced pressure to give the crude product. The crude product ethyl 5-(tert-butoxycarbonylamino)-4,5,6,7-tetrahydro-2-benzothiophene-1-carboxylate (6) (0.487 g) was obtained as a brown oil, and used into the next step without further purification.

### Procedure for preparation of ethyl 5-(tert-butoxycarbonylamino)-3-chloro-4,5,6,7-tetrahydro-2-benzothiophene-1-carboxylate (7)

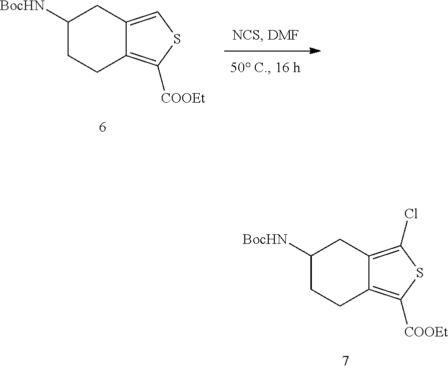

To a solution of ethyl 5-(tert-butoxycarbonylamino)-4,5,6,7-tetrahydro-2-benzothiophene-1-carboxylate (6) (0.34 g, 1.04 mmol, 1 eq) in DMF (3 mL) was added NCS (153.47 mg, 1.15 mmol, 1.1 eq). The mixture was stirred at 50° C. for 16 hr. The reaction mixture was then diluted with H₂O (10 mL) and extracted with EtOAc (10 mL*3). The combined organic layers were dried over Na₂SO₄, filtered and concentrated under reduced pressure to give a residue. The residue was purified by column chromatography (SiO₂, Petroleum ether/Ethyl acetate=5/1). Compound ethyl 5-(tert-butoxycarbonylamino)-3-chloro-4,5,6,7-tetrahydro-2-benzothiophene-1-carboxylate (7) (0.327 g, 763.28 μmol, 73.06% yield) was obtained as a yellow solid. ¹H NMR (400 MHz, CHLOROFORM-d) δ 4.62-4.47 (m, 1H), 4.36-4.25 (m, 2H), 4.03-3.88 (m, 1H), 3.22 (dt, J=18.89, 5.78 Hz, 1H), 3.06-2.93 (m, 2H), 2.41 (dd, J=16.44, 8.13 Hz, 1H), 2.07-1.94 (m, 1H), 1.82-1.68 (m, 1H), 1.49-1.40 (m, 9H), 1.36 (t, J=7.15 Hz, 3H)

### Procedure for preparation of 5-(tert-butoxycarbonylamino)-3-chloro-4,5,6,7-tetrahydro-2-benzothiophene-1-carboxylic acid (8)

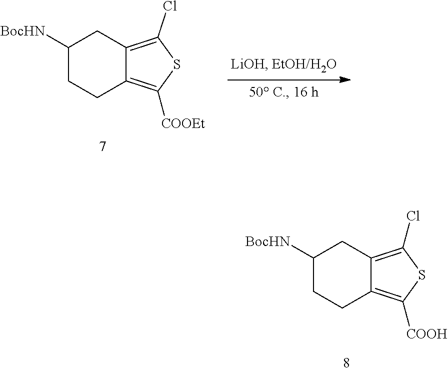 [SMILES: CCOC(=O)c1sc(Cl)c2c1CCC(NC(=O)OC(C)(C)C)C2 ; CC(C)(C)OC(=O)NC1CCc2c(C(=O)O)sc(Cl)c2C1]

To a solution of ethyl 5-(tert-butoxycarbonylamino)-3-chloro-4,5,6,7-tetrahydro-2-benzothiophene-1-carboxylate (7) (0.327 g, 908.67 μmol, 1 eq) in H₂O (3 mL) and EtOH (3 mL) was added lithium hydroxide hydrate (190.65 mg, 4.54 mmol, 5 eq). The mixture was stirred at 50° C. for 16 hr. The reaction mixture was diluted with H₂O (10 mL) and extracted with EtOAc (10 mL*3). The combined organic layers were washed with aq. sat. NaCl (10 mL*3), dried over Na₂SO₄, filtered and concentrated under reduced pressure to give a residue. The crude product 5-(tert-butoxycarbonylamino)-3-chloro-4,5,6,7-tetrahydro-2-benzothiophene-1-carboxylic acid (8) (0.251 g, crude) was obtained as a white solid, and used into the next step without further purification.

### Procedure for preparation of tert-butyl N-(3-chloro-4,5,6,7-tetrahydro-2-benzothiophen-5-yl)carbamate (9)

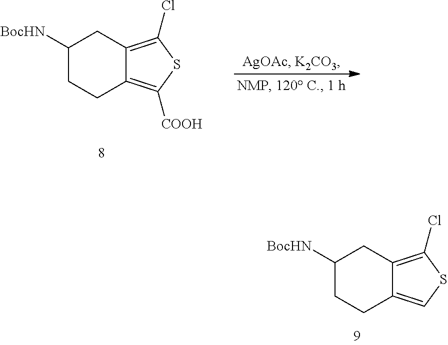 [SMILES: CC(C)(C)OC(=O)NC1CCc2c(C(=O)O)sc(Cl)c2C1 ; "AgOAc, K2CO3," ; CC(C)(C)OC(=O)NC1CCc2csc(Cl)c2C1]

A mixture of 5-(tert-butoxycarbonylamino)-3-chloro-4,5,6,7-tetrahydro-2-benzothiophene-1-carboxylic acid (8) (0.251 g, 756.45 μmol, 1 eq), AgOAc (12.63 mg, 75.64 μmol, 3.87 μL, 0.1 eq), and K₂CO₃ (209.10 mg, 1.51 mmol, 2 eq) in NMP (3 mL) was degassed and purged with N₂ for 3 times, and then the mixture was stirred at 120° C. for 1 hr under N₂ atmosphere. The reaction mixture was filtered, and the filtrate was partitioned between H₂O (10 mL) and EtOAc (10 mL). The organic phase was separated, washed with aq. sat. NaCl (5 mL*3), dried over Na₂SO₄, filtered and concentrated under reduced pressure to give a residue. The residue was purified by column chromatography (SiO2, Petroleum ether/Ethyl acetate=5/1). Compound tert-butyl N-(3-chloro-4,5,6,7-tetrahydro-2-benzothiophen-5-yl)carbamate (9) (0.157 g, 501.87 μmol, 66.35% yield) was obtained as a white solid. ¹H NMR (400 MHz, CHLOROFORM-d) δ 6.70 (s, 1H), 4.66-4.49 (m, 1H), 4.06-3.86 (m, 1H), 2.98 (dd, J=16.38, 5.25 Hz, 1H), 2.83-2.66 (m, 2H), 2.40-2.34 (m, 1H), 2.00-1.93 (m, 1H), 1.78-1.72 (m, 1H), 1.47 (s, 9H).

### Procedure for preparation of 3-chloro-4,5,6,7-tetrahydro-2-benzothiophen-5-amine hydrochloride (89)

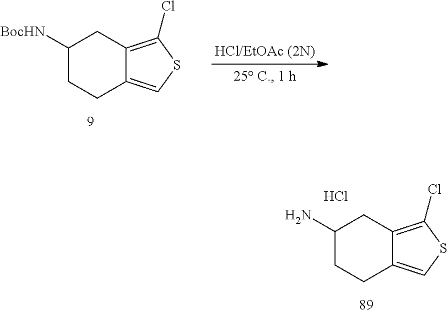 [SMILES: CC(C)(C)OC(=O)NC1CCc2csc(Cl)c2C1 ; NC1CCc2csc(Cl)c2C1 ; Cl]

To a solution of tert-butyl N-(3-chloro-4,5,6,7-tetrahydro-2-benzothiophen-5-yl)carbamate (9) (0.15 g, 521.19 μmol, 1 eq) in EtOAc (1 mL) was added HCl/EtOAc (1 mL). The mixture was stirred at 25° C. for 1 hr and then concentrated. The residue was purified by prep-HPLC (HCl condition, column: Welch Xtimate C18 150*25 mm*5 μm; mobile phase: [water(HCl)-ACN]; gradient:5%-35% B over 8 min). Compound 3-chloro-4,5,6,7-tetrahydro-2-benzothiophen-5-amine hydrochloride (89) (0.041 g, 179.25 μmol, 34.39% yield) was obtained as a white solid. LC-MS: m/z [M+H]⁺=188.1. ¹H NMR (400 MHz, METHANOL-d₄) δ 6.95 (s, 1H), 3.61-3.50 (m, 1H), 3.15 (ddd, J=16.23, 5.53, 1.38 Hz, 1H), 2.99-2.87 (m, 1H), 2.81-2.71 (m, 1H), 2.54 (dd, J=16.26, 10.01 Hz, 1H), 2.23-2.11 (m, 1H), 1.81 (dtd, J=12.85, 11.15, 11.15, 5.38 Hz, 1H).

### Synthetic scheme for preparation of 3-chloro-N-methyl-4,5,6,7-tetrahydrobenzothiophen-5-amine hydrochloride (90)

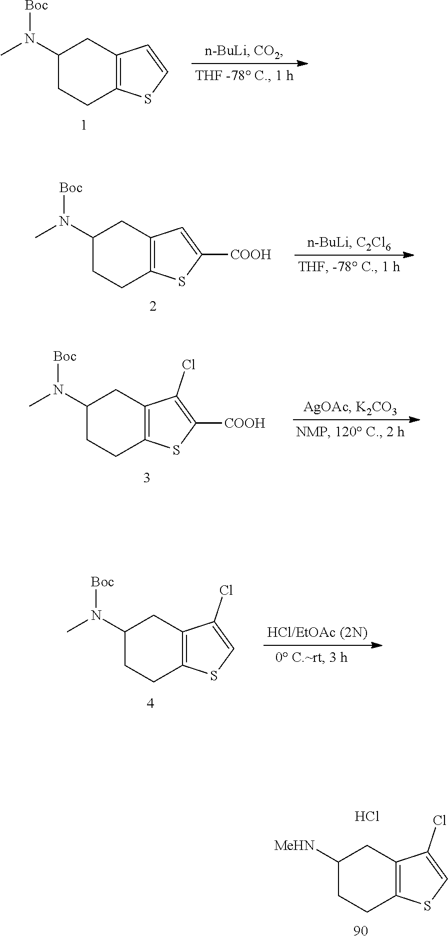 [SMILES: CN(C(=O)OC(C)(C)C)C1CCc2sccc2C1 ; CN(C(=O)OC(C)(C)C)C1CCc2sc(C(=O)O)cc2C1 ; CN(C(=O)OC(C)(C)C)C1CCc2sc(C(=O)O)c(Cl)c2C1 ; CN(C(=O)OC(C)(C)C)C1CCc2scc(Cl)c2C1 ; Cl ; CNC1CCc2scc(Cl)c2C1]

### Procedure for preparation of 5-[tert-butoxycarbonyl(methyl)amino]-4,5,6,7-tetrahydrobenzothiophene-2-carboxylic acid (2)

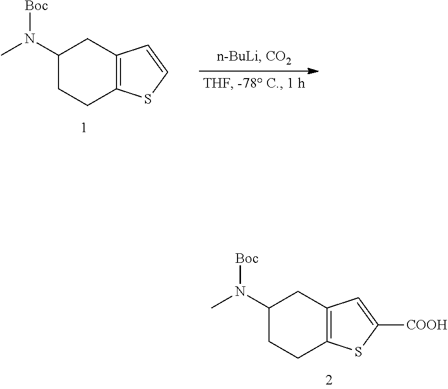 [SMILES: CN(C(=O)OC(C)(C)C)C1CCc2sccc2C1 ; CN(C(=O)OC(C)(C)C)C1CCc2sc(C(=O)O)cc2C1]

To a solution of tert-butyl N-methyl-N-(4,5,6,7-tetrahydrobenzothiophen-5-yl)carbamate (1) (4.2 g, 15.71 mmol, 1 eq) in THF (50 mL) was added n-BuLi (2.5 M, 10.05 mL, 1.6 eq) dropwise at −78° C., the mixture was stirred at −78~−60° C. for 30 min, then CO₂ (balloon, 15 psi) was bubbled to the solution for 30 min at −60° C. The reaction was quenched with water (50 mL) slowly, the aqueous layer was separated and acidified with 2N HCl to pH 5-6, then extracted with EA (30 mL*2). The combined organic layers were washed with brine (20 mL), dried over anhydrous Na₂SO₄, filtered and concentrated to get yellow solid, which was purified by silica gel column (PE:EtOAc=4:1 to 1:1) to afford 5-[tert-butoxycarbonyl(methyl)amino]-4,5,6,7-tetrahydro benzothiophene-2-carboxylic acid (2) (3 g, 9.63 mmol, 61.33% yield) as yellow solid.

### Procedure for preparation of 5-((tert-butoxycarbonyl)amino)-3-chloro-4,5,6,7-tetrahydrobenzo[b]thiophene-2-carboxylic acid (3)

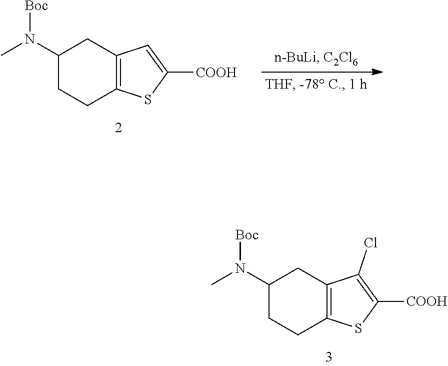 [SMILES: CN(C(=O)OC(C)(C)C)C1CCc2sc(C(=O)O)cc2C1 ; CN(C(=O)OC(C)(C)C)C1CCc2sc(C(=O)O)c(Cl)c2C1]

To a solution of 5-[tert-butoxycarbonyl(methyl)amino]-4,5,6,7-tetrahydrobenzothiophene-2-carboxylic acid (2) (2 g, 6.42 mmol, 1 eq) in THF (20 mL) was added n-BuLi (2.5 M, 6.01 mL, 2.34 eq), the mixture was stirred at −78° C. for 30 min, then 1,1,1,2,2,2-hexachloroethane (1.52 g, 6.42 mmol, 727.17 μL, 1 eq) in THF (4 mL) was added slowly, the mixture was stirred at −78-−60° C. for 30 min. The reaction was quenched with sat. aq. NH₄Cl (20 mL), H₂O (10 mL), sat. aq. citric acid (6 mL), extracted with EtOAc (20 mL). The organic layer was washed with brine (20 mL), dried over anhydrous Na₂SO₄, filtered and concentrated. The residue was purified by prep-HPLC (column: Phenomenex luna C18 (250*70 mm, 10 μm); mobile phase: [water(FA)-CAN]; gradient: 45%-75% B over 20 min) to afford the product 5-((tert-butoxycarbonyl)amino)-3-chloro-4,5,6,7-tetrahydrobenzo[b]thiophene-2-carboxylic acid (3) (1.8 g, 5.20 mmol) as a yellow solid.

### Procedure for preparation of tert-butyl N-(3-chloro-4,5,6,7-tetrahydrobenzothiophen-5-yl)-N-methyl-carbamate (4)

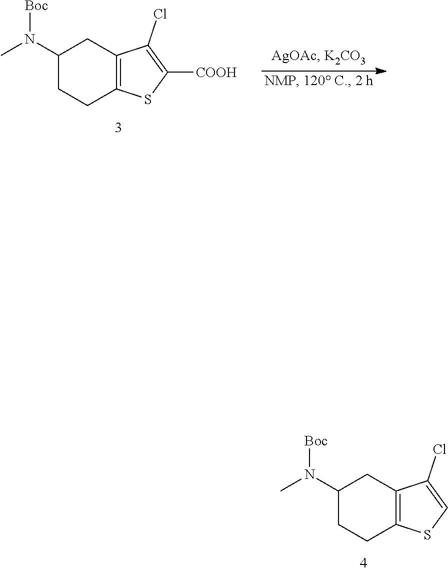 [SMILES: CN(C(=O)OC(C)(C)C)C1CCc2sc(C(=O)O)c(Cl)c2C1 ; "NMP, 120° C., 2 h" ; CN(C(=O)OC(C)(C)C)C1CCc2scc(Cl)c2C1]

A mixture of 5-[tert-butoxycarbonyl (methyl) amino]-3-chloro-4,5,6,7-tetrahydrobenzothiophene-2-carboxylic acid (3) (1.7 g, 4.92 mmol, 1 eq), AgOAc (205.12 mg, 1.23 mmol, 62.94 μL, 0.25 eq), K₂CO₃ (2.38 g, 17.20 mmol, 3.5 eq) in NMP (20 mL) was degassed and purged with N₂ for 3 times, and then the mixture was stirred at 120° C. for 2 hr under N₂ atmosphere. The reaction mixture was quenched by addition H₂O (15 mL) at 25° C., and then diluted with EtOAc (10 mL) and extracted with EtOAc (10 mL*3). The combined organic layers were washed with aq. sat. NaCl (10 mL*3), dried over Na₂SO₄, filtered and concentrated under reduced pressure to afford tert-butyl N-(3-chloro-4,5,6,7-tetrahydrobenzothiophen-5-yl)-N-methyl-carbamate (4) (460 mg, 1.52 mmol, 31.00% yield) as a yellow solid.

### Procedure for preparation of 3-chloro-N-methyl-4,5,6,7-tetrahydrobenzothiophen-5-amine hydrochloride (90)

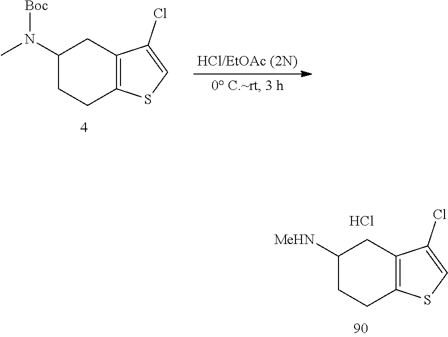 [SMILES: CN(C(=O)OC(C)(C)C)C1CCc2scc(Cl)c2C1 ; CNC1CCc2scc(Cl)c2C1 ; Cl]

To a solution of tert-butyl N-(3-chloro-4, 5, 6, 7-tetrahydrobenzothiophen-5-yl)-N-methyl-carbamate (4) (160 mg, 530.10 μmol, 1 eq) in EtOAc (2 mL) was added HCl/EtOAc (4 M, 2 mL, 15.09 eq) at 0° C. The mixture was stirred at 25° C. for 3 hr. The mixture was concentrated. The product was triturated with EtOAc at 25° C. for 10 min. The mixture was filtrered, the filter cake was dried to afford 3-chloro-N-methyl-4,5,6,7-tetrahydrobenzothiophen-5-amine hydrochloride (90) (54.65 mg, 227.16 μmol, 42.85% yield) as a white solid. ¹H NMR (400 MHz, METHANOL-d₄) δ=7.20 (s, 1H), 3.55 (dddd, J=2.9, 5.7, 8.5, 11.0 Hz, 1H), 3.11 (dd, J=5.4, 15.9 Hz, 1H), 3.03-2.88 (m, 2H), 2.82 (s, 3H), 2.64-2.54 (m, 1H), 2.39-2.31 (m, 1H), 1.96 (dddd, J=6.2, 9.7, 10.8, 12.9 Hz, 1H). LCMS: m/z [M+H]⁺=202.1.

### Synthetic scheme for preparation of N, 3-dimethyl-2-(trifluoromethyl)-4,5,6,7-tetrahydrobenzothiophen-6-amine hydrochloride (91)

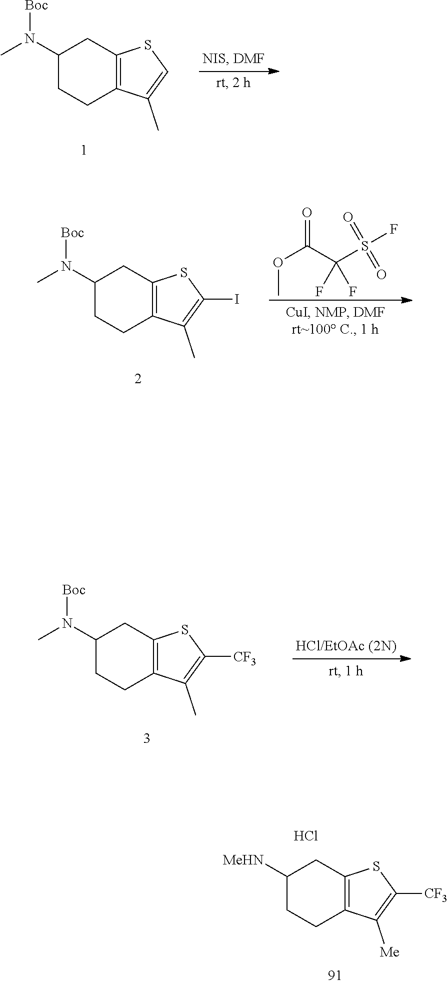

### Procedure for preparation of tert-butyl N-(2-iodo-3-methyl-4,5,6,7-tetrahydrobenzothiophen-6-yl)-N-methyl-carbamate (2)

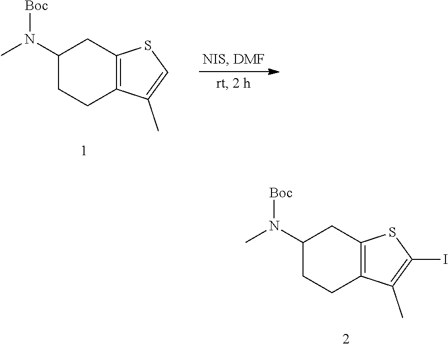 [SMILES: Cc1csc2c1CCC(N(C)C(=O)OC(C)(C)C)C2 ; Cc1c(I)sc2c1CCC(N(C)C(=O)OC(C)(C)C)C2]

To a solution of tert-butyl N-methyl-N-(3-methyl-4,5,6,7-tetrahydrobenzothiophen-6-yl) carbamate (1) (600 mg, 2.13 mmol, 1 eq) in DMF (10 mL) was added NIS (575.62 mg, 2.56 mmol, 1.2 eq). The mixture was stirred at 25° C. for 2 hr. The reaction mixture was poured into H₂O (10 mL) and extracted with EtOAc (10 mL*2). The combined organic phase was washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0-10% Ethyl acetate/Petroleum ether) to afford tert-butyl N-(2-iodo-3-methyl-4,5,6,7-tetrahydrobenzothiophen-6-yl)-N-methyl-carbamate (2) (860 mg, 2.11 mmol, 99.03% yield) as a yellow oil.

### Procedure for preparation of tert-butyl N-methyl-N-[3-methyl-2-(trifluoromethyl)-4,5,6,7-tetrahydrobenzothiophen-6-yl]carbamate (3)

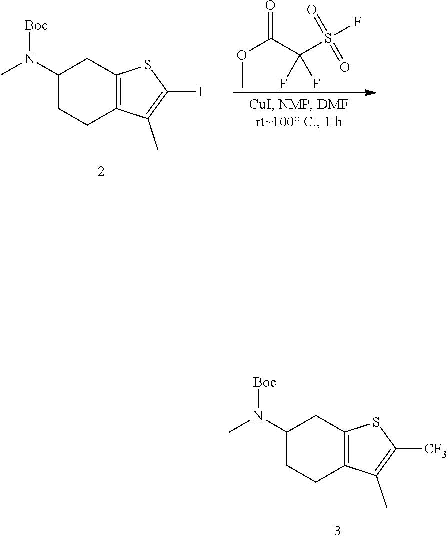

To a solution of tert-butyl N-(2-iodo-3-methyl-4,5,6,7-tetrahydrobenzothiophen-6-yl)-N-methyl-carbamate (2) (860 mg, 2.11 mmol, 1 eq) in DMF (10 mL) was added CuI (442.33 mg, 2.32 mmol, 1.1 eq) and NMP (1.05 g, 10.56 mmol, 1.03 mL, 5 eq) at 25° C. After addition, the mixture was stirred at this temperature for 10 min, and then methyl 2, 2-difluoro-2-fluorosulfonyl-acetate (2.03 g, 10.56 mmol, 1.34 mL, 5 eq) was added dropwise at 25° C. The resulting mixture was stirred at 100° C. for 1 hr. The reaction mixture was poured into H₂O (10 mL) and extracted with EtOAc (10 mL*2). The combined organic phase was washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~10% Ethyl acetate/Petroleum ether) to afford tert-butyl N-methyl-N-[3-methyl-2-(trifluoromethyl)-4,5,6,7-tetrahydrobenzothiophen-6-yl]carbamate (3) (660 mg, 1.89 mmol, 89.46% yield) as a red solid.

### Procedure for preparation of N, 3-dimethyl-2-(trifluoromethyl)-4,5,6,7-tetrahydrobenzothiophen-6-amine hydrochloride (91)

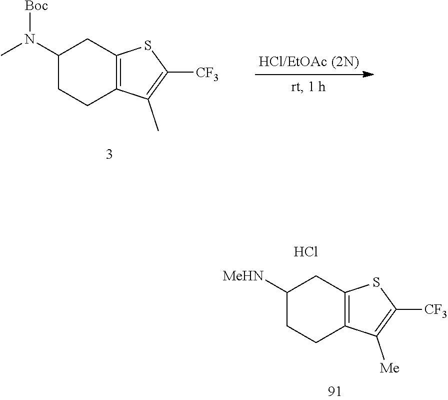 [SMILES: Cc1c(C(F)(F)F)sc2c1CCC(N(C)C(=O)OC(C)(C)C)C2 ; Cl ; CNC1CCc2c(sc(C(F)(F)F)c2C)C1]

To a solution of tert-butyl N-methyl-N-[3-methyl-2-(trifluoromethyl)-4,5,6,7-tetrahydrobenzothiophen-6-yl]carbamate (3) (100 mg, 286.20 μmol, 1 eq) in EtOAc (1 mL) was added HCl/EtOAc (4 M, 1 mL, 13.98 eq). The mixture was stirred at 25° C. for 1 hr. The reaction mixture was concentrated under reduced pressure to remove EtOAc. The residue was purified by prep-HPLC (column: Welch Xtimate C18 150*25 mm*5 μm; mobile phase: [water (HCl)-ACN]; gradient:14%-44% B over 8 min) to afford N, 3-dimethyl-2-(trifluoromethyl)-4,5,6,7-tetrahydrobenzothiophen-6-amine hydrochloride (91) (36.60 mg, 125.52 μmol, 43.86% yield) as a white solid. ¹H NMR (400 MHz, METHANOL-d₄) δ 3.65-3.52 (m, 1H), 3.35 (br s, 1H), 2.99-2.86 (m, 1H), 2.84-2.61 (m, 5H), 2.38-2.27 (m, 1H), 2.22 (d, J=1.5 Hz, 3H), 2.04-1.89 (m, 1H). LCMS: m/z [M+H]⁺=250.5.

### Synthetic scheme for preparation of 2-chloro-N, 3-dimethyl-4,5,6,7-tetrahydrobenzothiophen-6-amine hydrochloride (92)

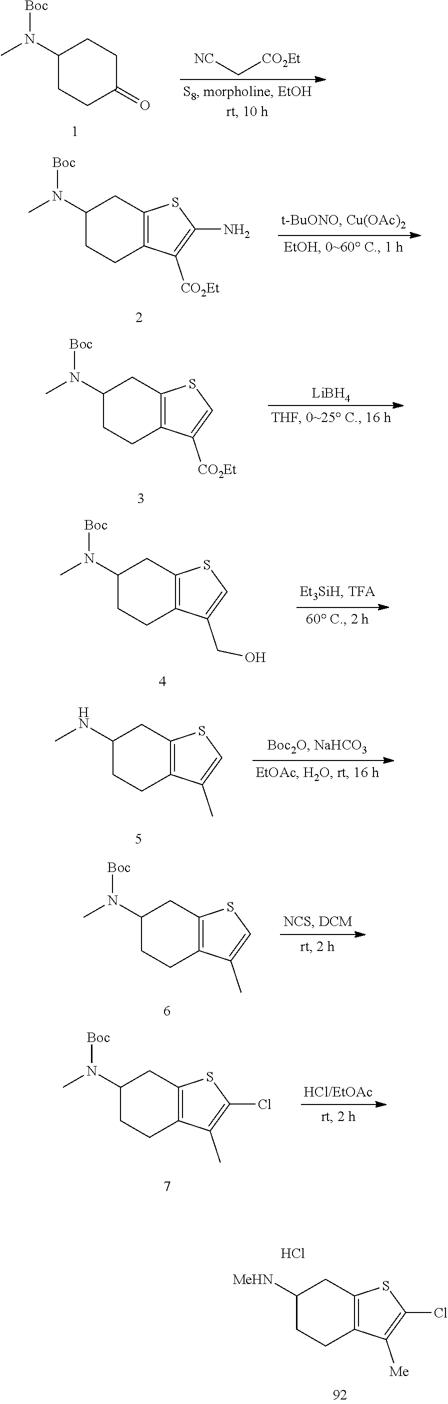

### Procedure for preparation of ethyl 2-amino-6-[tert-butoxycarbonyl (methyl)amino]-4,5,6,7-tetrahydrobenzothiophene-3-carboxylate (2)

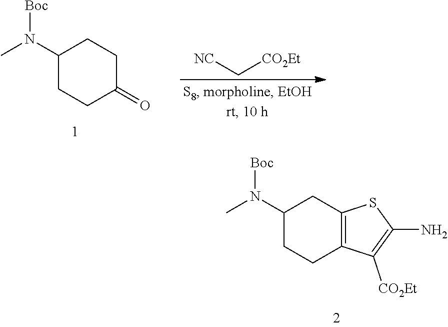 [SMILES: CN(C(=O)OC(C)(C)C)C1CCC(=O)CC1 ; CCOC(=O)CC#N ; CCOC(=O)c1c(N)sc2c1CCC(N(C)C(=O)OC(C)(C)C)C2]

To a solution of tert-butyl N-methyl-N-(4-oxocyclohexyl) carbamate (1) (10 g, 43.99 mmol, 1 eq) in EtOH (150 mL) was added ethyl 2-cyanoacetate (5.97 g, 52.79 mmol, 5.63 mL, 1.2 eq) and S (1.69 g, 52.79 mmol, 1.2 eq) and morpholine (4.22 g, 48.39 mmol, 4.26 mL, 1.1 eq). The mixture was stirred at 20° C. for 10 hr. The reaction was concentrated under reduced pressure to remove EtOH. Then the reaction mixture was added H₂O 100 mL at 0° C. and then extracted with EtOAc (300 mL*2), the combined organic layers were washed with brine (100 mL), dried over anhydrous Na₂SO₄, filtered and concentrated under reduced pressure. The residue was purified by column chromatography (SiO₂, Petroleum ether/Ethyl acetate=3/1 to 1/1) to afford ethyl 2-amino-6-[tert-butoxycarbonyl(methyl)amino]-4,5,6,7-tetrahydrobenzothiophene-3-carboxylate (2) (12.7 g, 35.83 mmol, 81.44% yield) as a red solid.

### Procedure for preparation of ethyl 6-[tert-butoxycarbonyl(methyl)amino]-4,5,6,7-tetrahydrobenzothiophene-3-carboxylate (3)

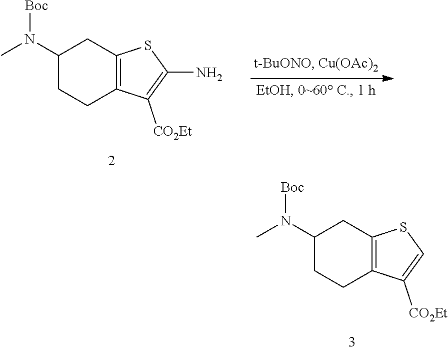 [SMILES: CCOC(=O)c1c(N)sc2c1CCC(N(C)C(=O)OC(C)(C)C)C2 ; CCOC(=O)c1csc2c1CCC(N(C)C(=O)OC(C)(C)C)C2]

To a solution of ethyl 2-amino-6-[tert-butoxycarbonyl(methyl)amino]-4,5,6,7-tetrahydrobenzothiophene-3-carboxylate (4.7 g, 13.26 mmol, 1 eq) and Cu(OAc)₂ (3.61 g, 19.89 mmol, 1.5 eq) in EtOH (60 mL) was added dropwise t-BuONO (1.50 g, 14.59 mmol, 1.73 mL, 1.1 eq) at 0° C. The resulting mixture was stirred at 60° C. for 1 hr. The mixture was followed by filtration, the filtrate was concentrated. The residue was purified by flash silica gel chromatography (ISCO®; 40 g SepaFlash® Silica Flash Column, eluent 0~20% Ethyl acetate/Petroleum ether) to afford ethyl 6-[tert-butoxycarbonyl (methyl) amino]-4,5,6,7-tetrahydrobenzothiophene-3-carboxylate (2.7 g, 7.95 mmol, 59.99% yield) as a white solid.

### Procedure for preparation of tert-butyl N-[3-(hydroxymethyl)-4,5,6,7-tetrahydrobenzothiophen-6-yl]-N-methyl-carbamate (4)

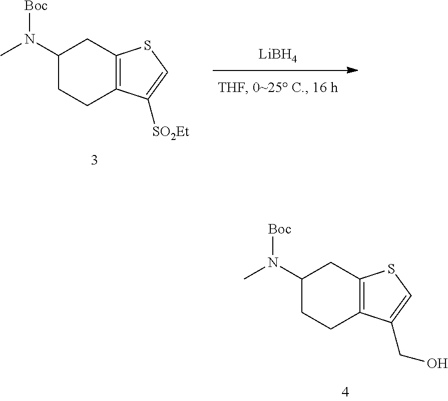

To a solution of ethyl 6-[tert-butoxycarbonyl(methyl)amino]-4,5,6,7-tetrahydrobenzothiophene-3-carboxylate (3) (2.7 g, 7.95 mmol, 1 eq) in THF (30 mL) was added LiBH₄ (2 M, 39.77 mL, 10 eq) at 0° C. The mixture was stirred at 0~25° C. for 16 hr. The reaction mixture was quenched with NH₄Cl aq. saturated solution 100 mL, extracted with EtOAc (50 mL*3). The combined organic layers were dried over Na₂SO₄, filtered and concentrated under reduced pressure. The crude product tert-butyl N-[3-(hydroxymethyl)-4,5,6,7-tetrahydrobenzothiophen-6-yl]-N-methyl-carbamate (4) (2.5 g, crude) was used into the next step without further purification.

### Procedure for preparation of N, 3-dimethyl-4,5,6,7-tetrahydrobenzothiophen-6-amine (5)

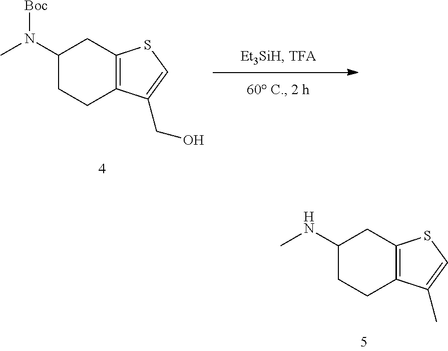

To a solution of tert-butyl N-[3-(hydroxymethyl)-4,5,6,7-tetrahydrobenzothiophen-6-yl]-N-methyl-carbamate (4) (3.3 g, 11.10 mmol, 1 eq) in DCM (10 mL) was added Et₃SiH (12.38 g, 106.43 mmol, 17 mL, 9.59 eq) and TFA (26.10 g, 228.86 mmol, 17 mL, 20.63 eq) at 25° C. Then the mixture was stirred at 60° C. for 2 hr. The reaction mixture was poured into NaHCO₃ saturated solution (50 mL). This crude mixture of N, 3-dimethyl-4,5,6,7-tetrahydrobenzothiophen-6-amine (5) was used into the next step without further purification.

### Procedure for preparation of tert-butyl N-methyl-N-(3-methyl-4,5,6,7-tetrahydrobenzothiophen-6-yl) carbamate (6)

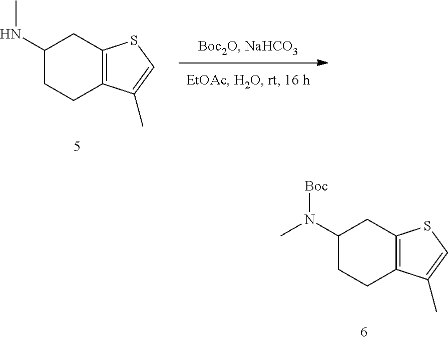

To the mixture of N, 3-dimethyl-4, 5, 6, 7-tetrahydrobenzothiophen-6-amine (5) obtained above was added EtOAc (30 mL), H₂O (15 mL) (Boc)₂O (4.82 g, 22.06 mmol, 5.07 mL, 2 eq) and NaHCO₃ (4.63 g, 55.16 mmol 5 eq). The mixture was stirred at 25° C. for 16 hr. The reaction mixture was poured into H₂O (30 mL) and extracted with EtOAc (30 mL*3). The combined organic phase was washed with brine (10 mL), dried over anhydrous sodium sulfate, filtered and concentrated under vacuum to give a residue. The residue was purified by flash silica gel chromatography (ISCO®; 40 g SepaFlash® Silica Flash Column, eluent 0~20% Ethyl acetate/Petroleum ether), and HPLC (column: Kromasil Eternity XT 250*80 mm*10 μm; mobile phase: [water (NH₄HCO₃)-ACN]; B %: 50%-80%, 20 min) to afford tert-butyl N-methyl-N-(3-methyl-4, 5, 6, 7-tetrahydrobenzothiophen-6-yl) carbamate (6) (950 mg, 3.38 mmol, 30.60% yield) as yellow oil.

### Procedure for preparation of tert-butyl N-(2-chloro-3-methyl-4,5,6,7-tetrahydrobenzothiophen-6-yl)-N-methyl-carbamate (7)

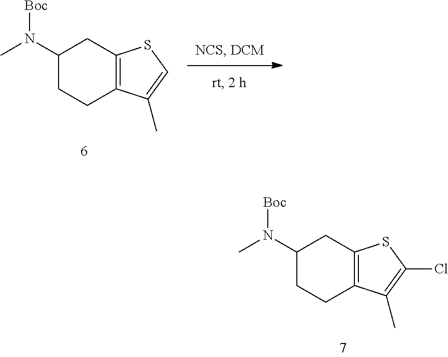

To a solution of tert-butyl N-methyl-N-(3-methyl-4,5,6,7-tetrahydrobenzothiophen-6-yl) carbamate (6) (300 mg, 1.07 mmol, 1 eq) in DCM (4 mL) was added NCS (170.82 mg, 1.28 mmol, 1.2 eq). The mixture was stirred at 25° C. for 2 hr. The reaction mixture was poured into H₂O (10 mL) and extracted with EtOAc (10 mL*2). The combined organic phase was washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~10% Ethyl acetate/Petroleum ether) to afford tert-butyl N-(2-chloro-3-methyl-4,5,6,7-tetrahydrobenzothiophen-6-yl)-N-methyl-carbamate (7) (240 mg, 759.84 μmol, 71.28% yield) as yellow oil.

### Procedure for preparation of 2-chloro-N, 3-dimethyl-4,5,6,7-tetrahydrobenzothiophen-6-amine hydrochloride (92)

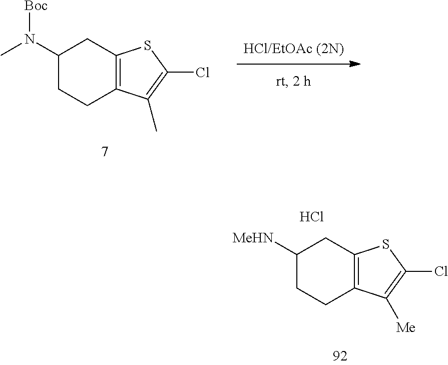 [SMILES: Cc1c(Cl)sc2c1CCC(N(C)C(=O)OC(C)(C)C)C2 ; "HCl/EtOAc (2N)" ; Cl ; CNC1CCc2c(sc(Cl)c2C)C1]

To a solution of tert-butyl N-(2-chloro-3-methyl-4,5,6,7-tetrahydrobenzothiophen-6-yl)-N-methyl-carbamate (7) (240 mg, 759.84 μmol, 1 eq) in EtOAc (1.5 mL) was added HCl/EtOAc (4 M, 2 mL, 10.53 eq). The mixture was stirred at 25° C. for 2 hr. The product was filtrered, the filter cake was collected and dried to afford 2-chloro-N, 3-dimethyl-4,5,6,7-tetrahydrobenzothiophen-6-amine hydrochloride (92) (156.61 mg, 602.34 μmol, 79.27% yield) as a white solid. ¹H NMR (400 MHz, METHANOL-d₄) δ=3.55 (dddd, J=2.9, 5.3, 8.1, 10.7 Hz, 1H), 3.24-3.15 (m, 1H), 2.85-2.76 (m, 4H), 2.74-2.55 (m, 2H), 2.34-2.24 (m, 1H), 2.05 (s, 3H), 2.00-1.86 (m, 1H) LCMS: m/z [M+H]⁺=216.1

### Synthetic scheme for preparation of 6-(methylamino)-4,5,6,7-tetrahydro-2-benzothiophene-1-carbonitrile hydrochloride (93)

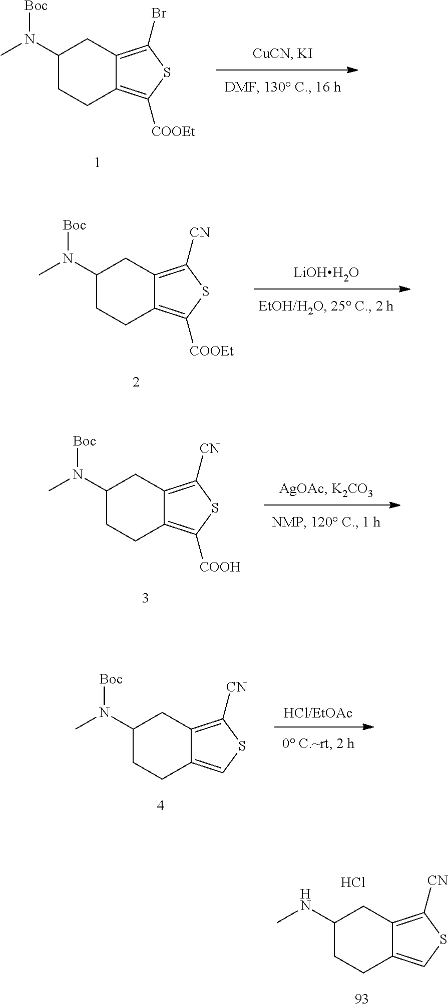

### Procedure for preparation of ethyl 5-[tert-butoxycarbonyl(methyl)amino]-3-cyano-4,5,6,7-tetrahydro-2-benzothiophene-1-carboxylate (2)

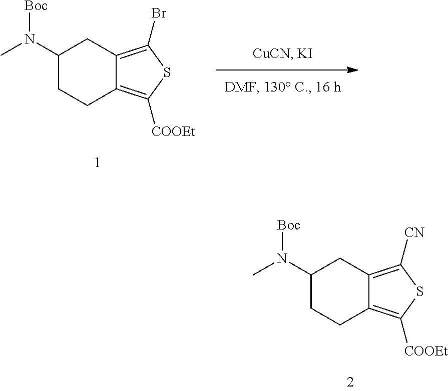 [SMILES: CCOC(=O)c1sc(Br)c2c1CCC(N(C)C(=O)OC(C)(C)C)C2 ; CCOC(=O)c1sc(C#N)c2c1CCC(N(C)C(=O)OC(C)(C)C)C2]

A mixture of ethyl 3-bromo-5-[tert-butoxycarbonyl(methyl)amino]-4,5,6,7-tetrahydro-2-benzothiophene-1-carboxylate (1) (1.7 g, 4.06 mmol, 1 eq), CuCN (1.09 g, 12.19 mmol, 2.66 mL, 3 eq), KI (134.91 mg, 812.73 μmol, 0.2 eq) in DMF (20 mL) was degassed and purged with N₂ for 3 times, and then the mixture was stirred at 130° C. for 16 hr under N₂ atmosphere. The reaction mixture was then concentrated under reduced pressure. Addition of H₂O (10 mL) at 25° C., and EtOAc (10 mL) followed. Aqueous layer extracted with EtOAc (10 mL*3). The combined organic layers were washed with aq. sat. NaCl 10 mL (5 mL*2), dried over Na₂SO₄, filtered and concentrated under reduced pressure. The residue was purified by flash silica gel chromatography (ISCO®; 20 g SepaFlash® Silica Flash Column, eluent 0~20% Ethyl acetate/Petroleum ether gradient @40 mL/min) to yield ethyl 5-[tert-butoxycarbonyl(methyl)amino]-3-cyano-4,5,6,7-tetrahydro-2-benzothiophene-1-carboxylate (2) (860 mg, 2.36 mmol, 58.07% yield) as a yellow solid. ¹H NMR (400 MHz, CHLOROFORM-d) δ 4.45-4.29 (m, 3H), 3.54-3.44 (m, 1H), 3.11-3.03 (m, 1H), 2.91-2.75 (m, 5H), 2.06-1.96 (m, 1H), 1.87-1.77 (m, 1H), 1.52-1.46 (m, 9H), 1.39 (t, J=7.1 Hz, 3H).

### Procedure for preparation of 5-[tert-butoxycarbonyl(methyl)amino]-3-cyano-4,5,6,7-tetrahydro-2-benzothiophene-1-carboxylic acid (3)

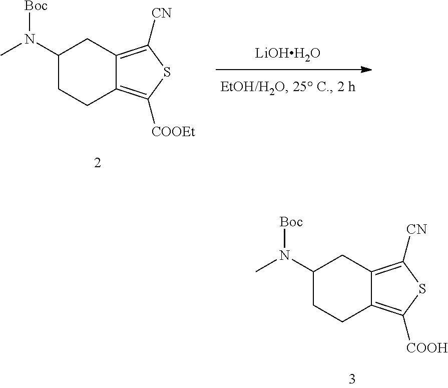 [SMILES: CCOC(=O)c1sc(C#N)c2c1CCC(N(C)C(=O)OC(C)(C)C)C2 ; "LiOH·H2O" ; CN(C(=O)OC(C)(C)C)C1CCc2c(C(=O)O)sc(C#N)c2C1]

A mixture of ethyl 5-[tert-butoxycarbonyl(methyl)amino]-3-cyano-4,5,6,7-tetrahydro-2-benzothiophene-1-carboxylate (2) (810 mg, 2.22 mmol, 1 eq), LiOH·H₂O (279.79 mg, 6.67 mmol, 3 eq) in EtOH (8 mL) and H₂O (0.8 mL) was stirred at 25° C. for 2 hr. The reaction mixture was quenched by addition H₂O (10 mL) and aq. sat. citric acid (8 ml) at 0° C., and then extracted with EtOAc (10 mL*3). The combined organic layers were washed with aq. sat. NaCl 10 mL (5 mL*2), dried over Na₂SO₄, filtered and concentrated under reduced pressure to give a residue. The crude product was purified by column chromatography (SiO₂, Petroleum ether/Ethyl acetate=1/0 to 5/1) to yield 5-[tert-butoxycarbonyl(methyl)amino]-3-cyano-4,5,6,7-tetrahydro-2-benzothiophene-1-carboxylic acid (3) as yellow oil. ¹H NMR (400 MHz, CHLOROFORM-d) δ 4.44-4.25 (m, 1H), 3.53-3.42 (m, 1H), 3.11-3.02 (m, 1H), 2.89-2.73 (m, 5H), 2.05-1.95 (m, 1H), 1.87-1.75 (m, 1H), 1.50 (s, 9H).

### Procedure for preparation of tert-butyl N-(3-cyano-4,5,6,7-tetrahydro-2-benzothiophen-5-yl)-N-methyl-carbamate (4)

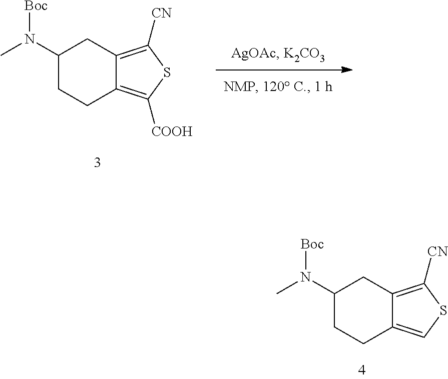

A mixture of 5-[tert-butoxycarbonyl(methyl)amino]-3-cyano-4,5,6,7-tetrahydro-2-benzothiophene-1-carboxylic acid (3) (400 mg, 1.19 mmol, 1 eq), AgOAc (39.69 mg, 237.81 μmol, 12.18 μL, 0.2 eq), K₂CO₃ (493.00 mg, 3.57 mmol, 3 eq) in NMP (8 mL) was degassed and purged with N₂ for 3 times, and then the mixture was stirred at 120° C. for 1 hr under N₂ atmosphere. The reaction mixture was quenched with H₂O 10 mL at 25° C., and then extracted with EtOAc (10 mL*3). The combined organic layers were washed with aq. sat. NaCl (10 mL*3), dried over Na₂SO₄, filtered and concentrated under reduced pressure. The crude product was purified by column chromatography (SiO2, Petroleum ether/Ethyl acetate=1/0 to 10/1) to obtain tert-butyl N-(3-cyano-4,5,6,7-tetrahydro-2-benzothiophen-5-yl)-N-methyl-carbamate (4) as a yellow solid. ¹H NMR (400 MHz, CHLOROFORM-d) δ 7.15 (s, 1H), 4.53-4.22 (m, 1H), 3.09 (dd, J=5.3, 16.9 Hz, 1H), 2.99-2.91 (m, 1H), 2.84-2.70 (m, 5H), 1.98-1.90 (m, 1H), 1.89-1.76 (m, 1H), 1.49 (s, 9H).

### Procedure for preparation of 6-(methylamino)-4,5,6,7-tetrahydro-2-benzothiophene-1-carbonitrile hydrochloride (93)

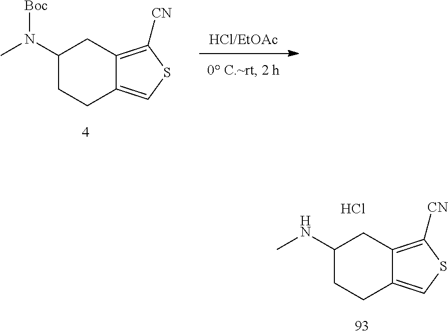 [SMILES: CN(C(=O)OC(C)(C)C)C1CCc2csc(C#N)c2C1 ; "HCl/EtOAc" ; CNC1CCc2csc(C#N)c2C1 ; Cl]

To a solution of tert-butyl N-(3-cyano-4,5,6,7-tetrahydro-2-benzothiophen-5-yl)-N-methyl-carbamate (4) (130 mg, 444.60 μmol, 1 eq) in EtOAc (1 mL) was added HCl/EtOAc (4 M, 1 mL, 9.00 eq) at 0° C. The mixture was stirred at 25° C. for 2 hr. The resulting product was concentrated. The crude product was triturated with acetonitrile at 25° C. for 2 min. Compound 6-(methylamino)-4,5,6,7-tetrahydro-2-benzothiophene-1-carbonitrile hydrochloride (93) (63.53 mg, 269.41 μmol, 60.59% yield) was obtained as a white solid. ¹H NMR (400 MHz, METHANOL-d4) δ 7.54 (s, 1H), 3.61-3.52 (m, 1H), 3.46 (ddd, J=1.4, 5.3, 16.6 Hz, 1H), 3.03 (td, J=4.6, 16.9 Hz, 1H), 2.93-2.77 (m, 5H), 2.39-2.27 (m, 1H), 1.89 (dtd, J=5.4, 11.1, 12.9 Hz, 1H). LCMS: m/z [M+H]⁺=193.2

### Synthetic scheme for preparation of 3-chloro-N-methyl-4,5,6,7-tetrahydrobenzothiophen-6-amine hydrochloride (94)

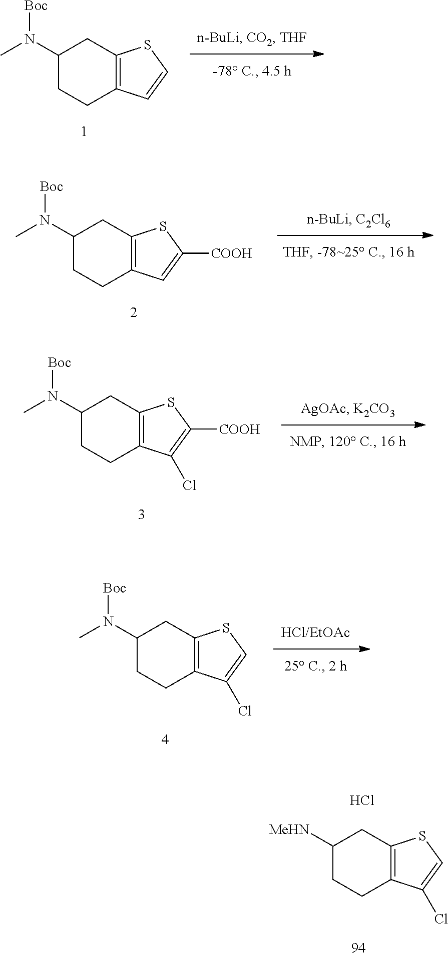 [SMILES: CN(C(=O)OC(C)(C)C)C1CCc2ccsc2C1 ; CN(C(=O)OC(C)(C)C)C1CCc2cc(C(=O)O)sc2C1 ; CN(C(=O)OC(C)(C)C)C1CCc2c(sc(C(=O)O)c2Cl)C1 ; CN(C(=O)OC(C)(C)C)C1CCc2c(Cl)csc2C1 ; Cl ; CNC1CCc2c(Cl)csc2C1]

### Procedure for preparation of 6-[tert-butoxycarbonyl(methyl)amino]-4,5,6,7-tetrahydrobenzothiophene-2-carboxylic acid (2)

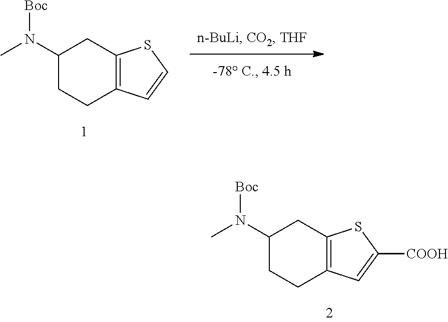 [SMILES: CN(C(=O)OC(C)(C)C)C1CCc2ccsc2C1 ; CN(C(=O)OC(C)(C)C)C1CCc2cc(C(=O)O)sc2C1]

To a mixture of tert-butyl N-methyl-N-(4,5,6,7-tetrahydrobenzothiophen-6-yl)carbamate (2) (2.3 g, 8.60 mmol, 1 eq) in THF (30 mL) was added n-BuLi (2.5 M, 4.13 mL, 1.2 eq) dropwise at −78° C. under N₂. The mixture was stirred at −78° C. for 0.5 hour. Then CO₂ (378.56 mg, 8.60 mmol, 1.5 μL, 1 eq) was bubbled into the mixture and purged with CO₂ (378.56 mg, 8.60 mmol, 1.5 μL, 1 eq) several times. The mixture was stirred under CO₂ (378.56 mg, 8.60 mmol, 1.5 μL, 1 eq) at −78° C. for 4 hours. The reaction mixture was poured into NH₄Cl saturated aq. solution (30 mL) and extracted with EtOAc (20 mL*2). The combined organic phases were washed with brine (10 mL), dried over anhydrous sodium sulfate, filtered and concentrated under vacuum. The residue was purified by flash silica gel chromatography (ISCO®; 40 g SepaFlash® Silica Flash Column, eluent 0~20% Ethyl acetate/Petroleum ether) to afford 6-[tert-butoxycarbonyl(methyl)amino]-4,5,6,7-tetrahydrobenzothiophene-2-carboxylic acid (2) (1.7 g, 5.46 mmol, 63.47% yield) as a yellow solid.

### Procedure for preparation of 6-[tert-butoxycarbonyl(methyl)amino]-3-chloro-4,5,6,7-tetrahydrobenzothiophene-2-carboxylic acid (3)

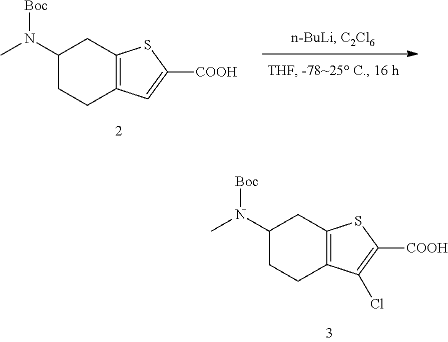 [SMILES: CN(C(=O)OC(C)(C)C)C1CCc2cc(C(=O)O)sc2C1 ; CN(C(=O)OC(C)(C)C)C1CCc2c(sc(C(=O)O)c2Cl)C1]

To a solution of 6-[tert-butoxycarbonyl(methyl)amino]-4,5,6,7-tetrahydrobenzothiophene-2-carboxylic acid (2) (910 mg, 2.92 mmol, 1 eq) in THF (10 mL) was added n-BuLi (2.5 M, 2.34 mL, 2 eq) dropwise over 15 min at −78° C. After addition, the mixture was stirred at this temperature for 15 min, and then 1,1,1,2,2,2-hexachloroethane (830.20 mg, 3.51 mmol, 397.03 μL, 1.2 eq) in THF (5 mL) was added dropwise at −78° C. The resulting mixture was stirred at −78−25° C. for 16 hr. The reaction mixture was poured into NH₄Cl saturated aq. solution (30 mL) and extracted with EtOAc (20 mL*2). The combined organic phase was washed with brine (10 mL), dried over anhydrous sodium sulfate, filtered and concentrated under vacuum. The residue was purified by prep-HPLC (column: Phenomenex Luna C18 150*40 mm*15 μm; mobile phase: [water (FA)-ACN]; gradient:45%-75% B over 11 min) to afford 6-[tert-butoxycarbonyl(methyl)amino]-3-chloro-4,5,6,7-tetrahydrobenzothiophene-2-carboxylic acid (3) (90 mg, 260.24 μmol, 8.91% yield) as a white solid.

### Procedure for preparation of tert-butyl N-(3-chloro-4,5,6,7-tetrahydrobenzothiophen-6-yl)-N-methyl-carbamate (4)

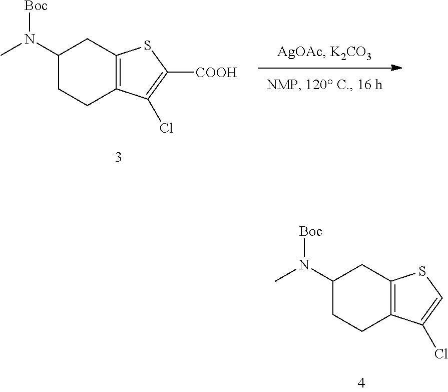 [SMILES: CN(C(=O)OC(C)(C)C)C1CCc2c(sc(C(=O)O)c2Cl)C1 ; CN(C(=O)OC(C)(C)C)C1CCc2c(Cl)csc2C1]

To a solution of 6-[tert-butoxycarbonyl(methyl)amino]-3-chloro-4,5,6,7-tetrahydrobenzothiophene-2-carboxylic acid (3) (160 mg, 462.64 μmol, 1 eq) in NMP (1 mL) was added AgOAc (15.44 mg, 92.53 μmol, 4.74 μL, 0.2 eq) and K₂CO₃ (191.82 mg, 1.39 mmol, 3 eq). The mixture was stirred at 120° C. for 16 hr. The mixture was followed by filtration, the filtrate was poured into H₂O (10 mL) and extracted with EtOAc (10 mL*3). The combined organic phases were washed with brine (10 mL), dried over anhydrous sodium sulfate, filtered and concentrated under vacuum to give a residue. The residue was purified by prep-HPLC (column: Phenomenex Luna C18 150*25 mm*10 μm; mobile phase: [water (FA)-ACN]; gradient:70%-100% B over 9 min) to afford tert-butyl N-(3-chloro-4,5,6,7-tetrahydrobenzothiophen-6-yl)-N-methyl-carbamate (4) (85 mg, 281.61 μmol, 60.87% yield) as a white solid.

### Procedure for preparation of 3-chloro-N-methyl-4,5,6,7-tetrahydrobenzothiophen-6-amine hydrochloride (94)

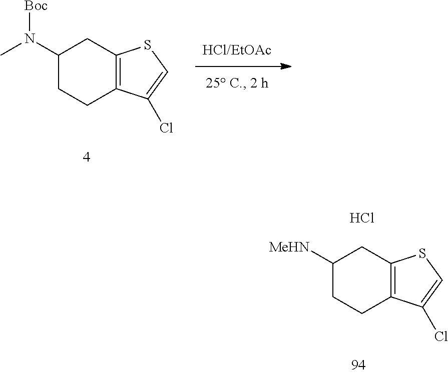 [SMILES: CN(C(=O)OC(C)(C)C)C1CCc2c(Cl)csc2C1 ; Cl ; CNC1CCc2c(Cl)csc2C1]

To a solution of tert-butyl N-(3-chloro-4,5,6,7-tetrahydrobenzothiophen-6-yl)-N-methyl-carbamate (4) (85 mg, 281.61 μmol, 1 eq) in EtOAc (1 mL) was added HCl/EtOAc (4 M, 1 mL, 14.20 eq). The mixture was stirred at 25° C. for 2 hr. The mixture was concentrated. The residue was triturated with EtOAc at 25° C. for 10 min. Followed filtration, the filter cake was dried to afford 3-chloro-N-methyl-4,5,6,7-tetrahydrobenzothiophen-6-amine hydrochloride (94) (58.47 mg, 240.58 μmol, 85.43% yield) as a white solid. ¹H NMR (400 MHz, METHANOL-d₄) δ=7.21 (s, 1H), 3.62-3.53 (m, 1H), 3.28 (br s, 1H), 2.96-2.61 (m, 6H), 2.38-2.25 (m, 1H), 2.06-1.90 (m, 1H). LCMS: m/z [M+H]⁺=202.2.

### Synthetic scheme for preparation of 3-cyclopropyl-N-methyl-4,5,6,7-tetrahydro-2-benzothiophen-5-amine hydrochloride (95)

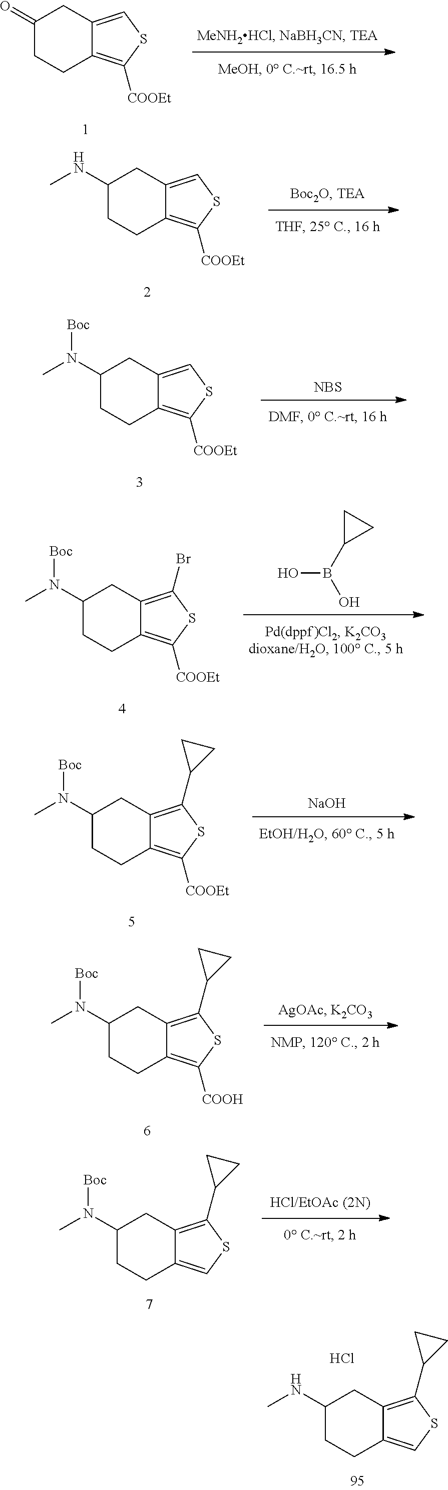

### Procedure for preparation of ethyl 5-(methylamino)-4,5,6,7-tetrahydro-2-benzothiophene-1-carboxylate (2)

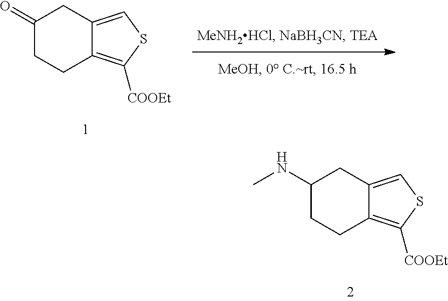 [SMILES: CCOC(=O)c1scc2c1CCC(=O)C2 ; CCOC(=O)c1scc2c1CCC(NC)C2]

To a solution of ethyl 5-oxo-6,7-dihydro-4H-2-benzothiophene-1-carboxylate (1) (4 g, 17.84 mmol, 1 eq), methylamine (3.61 g, 53.51 mmol, 3 eq, HCl), TEA (3.61 g, 35.67 mmol, 4.96 mL, 2 eq) in MeOH (100 mL) stirred at 0° C. for 0.5 h, then was added NaBH₃CN (5.60 g, 89.18 mmol, 5 eq) slowly at 0° C. The mixture was stirred at 0~25° C. for 16 hr. The reaction mixture was quenched by addition NaHCO₃ aq. saturated solution (40 mL) at 0° C., and then the mixture was concentrated under reduced pressure to remove MeOH, the mixture was extracted with EtOAc (30 mL*3). The combined organic layers were washed with NaCl saturated aq. solution (50 mL*2), dried over Na₂SO₄, filtered and concentrated under reduced pressure to give a residue. The crude product ethyl 5-(methylamino)-4,5,6,7-tetrahydro-2-benzothiophene-1-carboxylate (2) (4.7 g, crude) was obtained as a yellow solid.

### Procedure for preparation of ethyl 5-[tert-butoxycarbonyl(methyl)amino]-4,5,6,7-tetrahydro-2-benzothiophene-1-carboxylate (3)

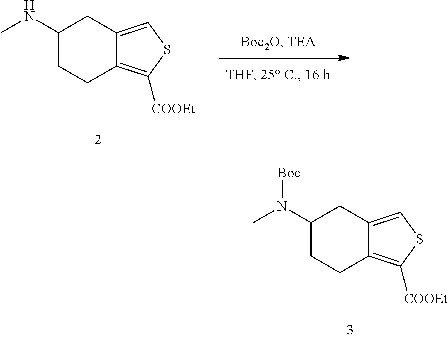 [SMILES: CCOC(=O)c1scc2c1CCC(NC)C2 ; CCOC(=O)c1scc2c1CCC(N(C)C(=O)OC(C)(C)C)C2]

To a solution of ethyl 5-(methylamino)-4,5,6,7-tetrahydro-2-benzothiophene-1-carboxylate (2) (4.7 g, 19.64 mmol, 1 eq) in THF (60 mL) was added Boc₂O (5.57 g, 25.53 mmol, 5.86 mL, 1.3 eq) and TEA (3.97 g, 39.28 mmol, 5.47 mL, 2 eq). The mixture was stirred at 25° C. for 16 hr. The reaction mixture was quenched by addition H₂O (50 mL) at 25° C., and extracted with EtOAc (40 mL*3). The combined organic layers were dried over Na₂SO₄, filtered and concentrated under reduced pressure. The crude product was purified by flash silica gel chromatography (ISCO®; 25 g SepaFlash® Silica Flash Column, eluent 0~25% Ethylacetate/Petroleum ether gradient @40 mL/min) to afford ethyl 5-[tert-butoxycarbonyl(methyl)amino]-4,5,6,7-tetrahydro-2-benzothiophene-1-carboxylate (3) (4.9 g, 14.44 mmol, 73.51% yield) as a yellow solid. ¹H NMR (400 MHz, CHLOROFORM-d) δ=7.09 (s, 1H), 4.32 (q, J=7.1 Hz, 3H), 3.54-3.46 (m, 1H), 2.92-2.79 (m, 5H), 2.76-2.67 (m, 1H), 1.99-1.92 (m, 1H), 1.81 (dq, J=5.4, 12.4 Hz, 1H), 1.48 (s, 9H), 1.37 (t, J=7.1 Hz, 3H)

### Procedure for preparation of ethyl 3-bromo-5-[tert-butoxycarbonyl(methyl)amino]-4,5,6,7-tetrahydro-2-benzothiophene-1-carboxylate (4)

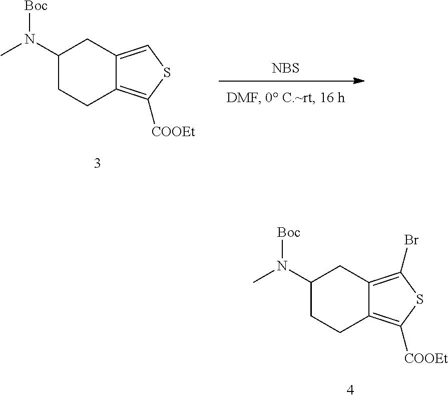 [SMILES: CCOC(=O)c1scc2c1CCC(N(C)C(=O)OC(C)(C)C)C2 ; "NBS" ; CCOC(=O)c1sc(Br)c2c1CCC(N(C)C(=O)OC(C)(C)C)C2]

To a solution of ethyl 5-[tert-butoxycarbonyl(methyl)amino]-4,5,6,7-tetrahydro-2-benzothiophene-1-carboxylate (3) (1 g, 2.95 mmol, 1 eq) in DMF (10 mL) was added NBS (629.20 mg, 3.54 mmol, 1.2 eq) slowly at 0° C., the mixture was stirred at 0-25° C. for 16 hr. The reaction mixture was quenched by addition of H₂O (10 mL) at 25° C., and then extracted with EtOAc (10 mL*3). The combined organic layers were washed with NaCl saturated aq. solution (10 mL*3), dried over Na₂SO₄, filtered and concentrated under reduced pressure to give a residue. The crude product was purified by column chromatography (SiO₂, Petroleum ether/Ethyl acetate=10/1 to 5/1) to yield ethyl 3-bromo-5-[tert-butoxycarbonyl(methyl)amino]-4,5,6,7-tetrahydro-2-benzothiophene-1-carboxylate (4) (1.1 g, 2.63 mmol, 89.25% yield) as a yellow solid. ¹H NMR (400 MHz, CHLOROFORM-d) δ=4.43-4.22 (m, 3H), 3.55-3.46 (m, 1H), 2.89-2.72 (m, 5H), 2.48 (br dd, J=11.7, 16.2 Hz, 1H), 1.99-1.88 (m, 1H), 1.81-1.68 (m, 1H), 1.49 (s, 9H), 1.39-1.32 (m, 3H)

### Procedure for preparation of ethyl 5-[tert-butoxycarbonyl(methyl)amino]-3-cyclopropyl-4,5,6,7-tetrahydro-2-benzothiophene-1-carboxylate (5)

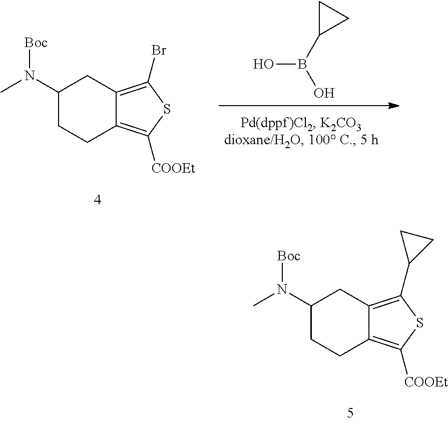 [SMILES: CCOC(=O)c1sc(Br)c2c1CCC(N(C)C(=O)OC(C)(C)C)C2 ; OB(O)C1CC1 ; CCOC(=O)c1sc(C2CC2)c2c1CCC(N(C)C(=O)OC(C)(C)C)C2]

To a solution of ethyl 3-bromo-5-[tert-butoxycarbonyl(methyl)amino]-4,5,6,7-tetrahydro-2-benzothiophene-1-carboxylate (4) (900 mg, 2.15 mmol, 1 eq) and cyclopropylboronic acid (923.97 mg, 10.76 mmol, 5 eq) in dioxane (20 mL) was added H₂O (2 mL), Pd(dppf)Cl₂ (157.41 mg, 215.13 μmol, 0.1 eq) and K₂CO₃ (594.65 mg, 4.30 mmol, 2 eq). The mixture was stirred at 100° C. for 5 hr under N₂ atmosphere. The reaction mixture was quenched by addition of H₂O (20 mL) at 25° C., and extracted with EtOAc (20 mL*3). The combined organic layers were dried over Na₂SO₄, filtered and concentrated under reduced pressure to give a residue. The crude product was purified by column chromatography (SiO₂, Petroleum ether/Ethyl acetate=1/0 to 10/1) to yield ethyl 5-[tert-butoxycarbonyl(methyl)amino]-3-cyclopropyl-4,5,6,7-tetrahydro-2-benzothiophene-1-carboxylate (5) (680 mg, 1.79 mmol, 83.29% yield) as yellow oil. ¹H NMR (400 MHz, CHLOROFORM-d) δ=4.48-4.20 (m, 3H), 3.52-3.41 (m, 1H), 2.94-2.75 (m, 5H), 2.65-2.56 (m, 1H), 1.98-1.89 (m, 2H), 1.79 (dq, J=5.3, 12.4 Hz, 1H), 1.52-1.47 (m, 9H), 1.05 (br dd, J=2.9, 7.6 Hz, 2H), 0.77-0.71 (m, 2H).

### Procedure for preparation of 5-[tert-butoxycarbonyl(methyl)amino]-3-cyclopropyl-4,5,6,7-tetrahydro-2-benzothiophene-1-carboxylic acid (6)

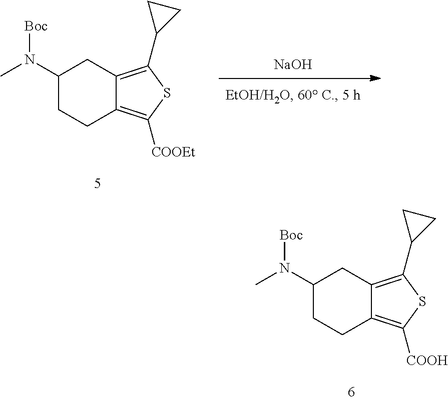 [SMILES: CCOC(=O)c1sc(C2CC2)c2c1CCC(N(C)C(=O)OC(C)(C)C)C2 ; "NaOH" ; CN(C(=O)OC(C)(C)C)C1CCc2c(C(=O)O)sc(C3CC3)c2C1]

To a solution of ethyl 5-[tert-butoxycarbonyl(methyl)amino]-3-cyclopropyl-4,5,6,7-tetrahydro-2-benzothiophene-1-carboxylate (5) (680 mg, 1.79 mmol, 1 eq) in EtOH (10 mL) was added NaOH (143.33 mg, 3.58 mmol, 2 eq) and H₂O (2 mL). The mixture was stirred at 60° C. for 3 hr. NaOH (71.67 mg, 1.79 mmol, 1 eq) was added, the mixture was stirred at 60° C. for 2 hr. The reaction mixture was quenched by addition of aq. 2N HCl (2 mL) and H₂O (5 mL) at 0° C., and then extracted with EtOAc (10 mL*3). The combined organic layers were washed with NaCl aq. saturated solution 10 mL (5 mL*2), dried over Na₂SO₄, filtered and concentrated under reduced pressure. 5-[tert-butoxycarbonyl(methyl)amino]-3-cyclopropyl-4,5,6,7-tetrahydro-2-benzothiophene-1-carboxylic acid (6) (460 mg, 1.31 mmol, 73.05% yield) was obtained as a yellow solid. ¹H NMR (400 MHz, CHLOROFORM-d) δ=4.37 (br s, 1H), 3.51-3.42 (m, 1H), 2.95-2.75 (m, 5H), 2.66-2.57 (m, 1H), 2.00-1.90 (m, 2H), 1.80 (dq, J=5.3, 12.5 Hz, 1H), 1.52-1.45 (m, 9H), 1.15-0.99 (m, 2H), 0.83-0.70 (m, 2H)

### Procedure for preparation of tert-butyl N-(3-cyclopropyl-4,5,6,7-tetrahydro-2-benzothiophen-5-yl)-N-methyl-carbamate (7)

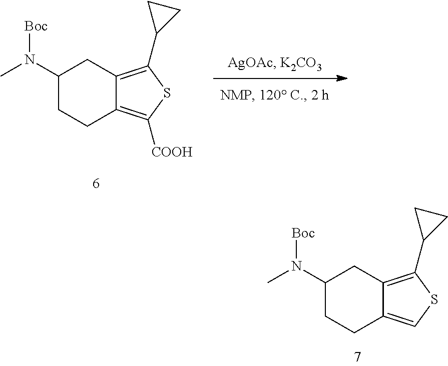

To a solution of 5-[tert-butoxycarbonyl(methyl)amino]-3-cyclopropyl-4,5,6,7-tetrahydro-2-benzothiophene-1-carboxylic acid (6) (410 mg, 1.17 mmol, 1 eq) in NMP (8 mL) was added AgOAc (38.94 mg, 233.31 μmol, 11.95 μL, 0.2 eq) and K₂CO₃ (483.68 mg, 3.50 mmol, 3 eq). The mixture was stirred at 120° C. for 2 hr. The reaction mixture was quenched by H₂O (10 mL) at 25° C., and then extracted with EtOAc (10 mL*3). The combined organic layers were washed with NaCl saturated aq. solution (5 mL*2), dried over Na₂SO₄, filtered and concentrated under reduced pressure to give a residue. The crude product was purified by column chromatography (SiO₂, Petroleum ether/Ethyl acetate=1/0 to 10/1) to yield tert-butyl N-(3-cyclopropyl-4,5,6,7-tetrahydro-2-benzothiophen-5-yl)-N-methyl-carbamate (7) (360 mg, crude) as yellow oil. ¹H NMR (400 MHz, CHLOROFORM-d) δ=6.58 (s, 1H), 4.55-4.21 (m, 1H), 2.95-2.78 (m, 5H), 2.78-2.68 (m, 1H), 2.65-2.48 (m, 1H), 1.95-1.75 (m, 3H), 1.49 (s, 9H), 1.02-0.91 (m, 2H), 0.70-0.62 (m, 2H)

### Procedure for preparation of 3-cyclopropyl-N-methyl-4,5,6,7-tetrahydro-2-benzothiophen-5-amine hydrochloride (95)

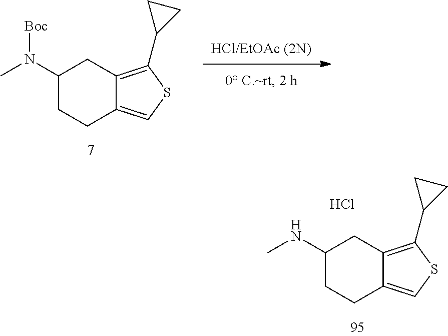 [SMILES: CN(C(=O)OC(C)(C)C)C1CCc2csc(C3CC3)c2C1 ; "HCl/EtOAc (2N)" ; CNC1CCc2csc(C3CC3)c2C1 ; Cl]

To a solution of tert-butyl N-(3-cyclopropyl-4,5,6,7-tetrahydro-2-benzothiophen-5-yl)-N-methyl-carbamate (7) (360 mg, 1.17 mmol, 1 eq) in EtOAc (3 mL) was added HCl/EtOAc (4 M, 3 mL, 10.25 eq). The mixture was stirred at 0~25° C. for 2 hr. The mixture was concentrated in vacuo. Compound 3-cyclopropyl-N-methyl-4,5,6,7-tetrahydro-2-benzothiophen-5-amine hydrochloride (95) (83.4 mg, 390.18 μmol, 33.32% yield) was obtained as a yellow solid. ¹H NMR (400 MHz, METHANOL-d4) δ=6.73 (s, 1H), 3.47 (tdd, J=2.8, 10.2, 13.0 Hz, 1H), 3.34 (br d, J=5.5 Hz, 1H), 2.95-2.87 (m, 1H), 2.81 (s, 3H), 2.77-2.63 (m, 2H), 2.27-2.20 (m, 1H), 2.02-1.94 (m, 1H), 1.88-1.77 (m, 1H), 1.02-0.97 (m, 2H), 0.65-0.61 (m, 2H). LCMS: m/z [M+H]⁺=207.9

### Synthetic scheme for preparation of 2-chloro-N-methyl-4,5,6,7-tetrahydrobenzothiophen-6-amine hydrochloride (96)

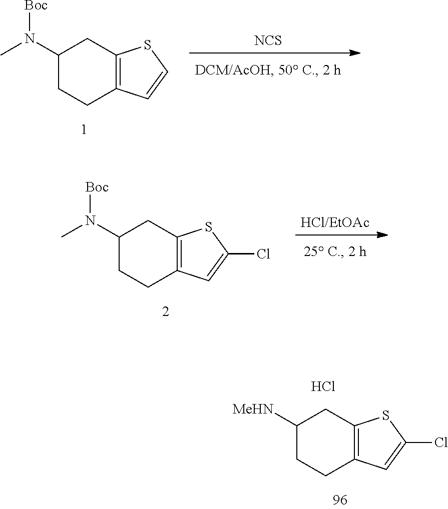 [SMILES: CN(C(=O)OC(C)(C)C)C1CCc2ccsc2C1 ; "NCS" ; CN(C(=O)OC(C)(C)C)C1CCc2cc(Cl)sc2C1 ; "HCl/EtOAc" ; Cl ; CNC1CCc2cc(Cl)sc2C1]

### Procedure for preparation of tert-butyl N-(2-chloro-4,5,6,7-tetrahydrobenzothiophen-6-yl)-N-methyl-carbamate (2)

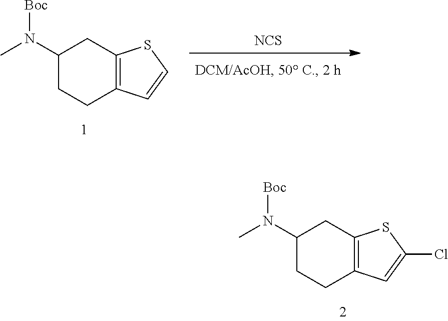 [SMILES: CN(C(=O)OC(C)(C)C)C1CCc2ccsc2C1 ; "NCS" ; CN(C(=O)OC(C)(C)C)C1CCc2cc(Cl)sc2C1]

To a mixture of tert-butyl N-methyl-N-(4,5,6,7-tetrahydrobenzothiophen-6-yl) carbamate (1) (400 mg, 1.50 mmol, 1 eq) in DCM (2 mL) and AcOH (1 mL) was added NCS (239.71 mg, 1.80 mmol, 1.2 eq) under N₂. The mixture was stirred at 50° C. for 2 hours. The reaction mixture was quenched by addition NaHCO₃ (aq, 100 mL), mixture was extracted with EtOAc (50 mL*3). The combined organic phases were washed with brine (50 mL*3), dried over Na₂SO₄, filtered and concentrated at reduced pressure. The residue was purified by silica gel chromatography (100-200 mesh silica gel, Petroleum ether/Ethyl acetate=1/0~10/1, Rf=0.55) to afford tert-butyl N-(2-chloro-4,5,6,7-tetrahydrobenzothiophen-6-yl)-N-methyl-carbamate (2) (350 mg, 1.16 mmol, 77.51% yield) as a white solid. LCMS: m/z [M+H-tBu]⁺=246.1.

### Procedure for preparation of 2-chloro-N-methyl-4,5,6,7-tetrahydrobenzothiophen-6-amine hydrochloride (96)

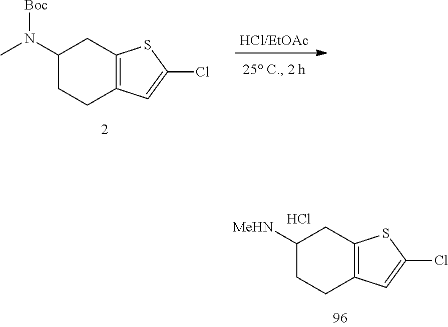 [SMILES: CN(C(=O)OC(C)(C)C)C1CCc2cc(Cl)sc2C1 ; "HCl/EtOAc" ; Cl ; CNC1CCc2cc(Cl)sc2C1]

To a solution of tert-butyl N-(2-chloro-4,5,6,7-tetrahydrobenzothiophen-6-yl)-N-methyl-carbamate (2) (100 mg, 331.31 μmol, 1 eq) in EtOAc (4 mL) was added HCl/EtOAc (4 mL). The mixture was stirred at 25° C. for 2 hr. The reaction mixture was concentrated under reduced pressure to give a residue. The residue was purified by prep-HPLC (column: Welch Xtimate C18 150*25 mm*5 μm; mobile phase: [water (HCl)-ACN]; gradient: 5%-35% B over 8 min) to afford 2-chloro-N-methyl-4,5,6,7-tetrahydrobenzothiophen-6-amine hydrochloride (96) (46.40 mg, 194.81 μmol, 58.80% yield) as a white solid. LCMS: m/z [M+H]⁺=202.1. ¹H NMR (400 MHz, Methanol-d4, 297 K) δ (ppm)=6.71 (s, 1H), 3.55 (dddd, J=2.9, 5.4, 8.3, 10.9 Hz, 1H), 3.22 (dd, J=5.2, 16.1 Hz, 1H), 2.86-2.66 (m, 6H), 2.31-2.21 (m, 1H), 1.98-1.85 (m, 1H)

### Synthetic scheme for preparation of 2-chloro-N-methyl-4,5,6,7-tetrahydrobenzothiophen-5-amine hydrochloride (97)

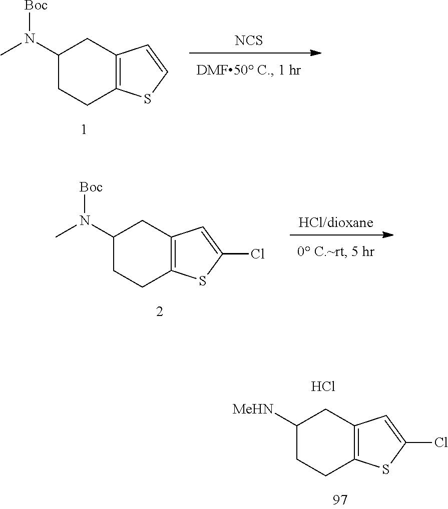 [SMILES: CN(C(=O)OC(C)(C)C)C1CCc2sccc2C1 ; "NCS" ; CN(C(=O)OC(C)(C)C)C1CCc2sc(Cl)cc2C1 ; "HCl/dioxane" ; Cl ; CNC1CCc2sc(Cl)cc2C1]

### Procedure for preparation of tert-butyl N-(2-chloro-4,5,6,7-tetrahydrobenzothiophen-5-yl)-N-methyl-carbamate (2)

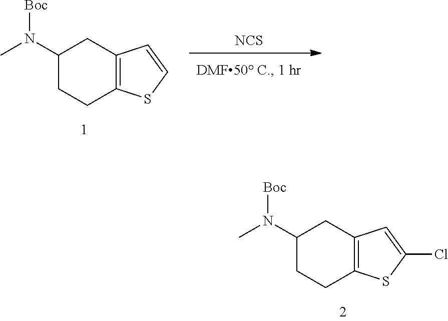 [SMILES: CN(C(=O)OC(C)(C)C)C1CCc2sccc2C1 ; "NCS" ; CN(C(=O)OC(C)(C)C)C1CCc2sc(Cl)cc2C1]

To a solution of tert-butyl N-methyl-N-(4,5,6,7-tetrahydrobenzothiophen-5-yl)carbamate (1) (0.9 g, 3.37 mmol, 1 eq) in DMF (10 mL) was added NCS (561.81 mg, 4.21 mmol, 1.25 eq). The mixture was stirred at 50° C. for 1 hr. The reaction mixture was poured into H₂O (50 mL). The reaction was diluted with NH₃·H₂O (60 mL) and extracted with EtOAc mL (30 mL*3). The combined organic layers were washed with brine (20 mL*3), dried over anhydrous sodium sulfate, filtered and concentrated under reduced pressure. The residue was purified by flash silica gel chromatography (ISCO®; 4 g SepaFlash® Silica Flash Column, eluent 0-20% Ethyl acetate/Petroleum ether gradient @60 mL/min) and prep-HPLC (column: Phenomenex luna C18 150*25 mm*10 μm; mobile phase: [water(HCl)-ACN]; gradient:60%-90% B over 10 min). Compound tert-butyl N-(2-chloro-4,5,6,7-tetrahydrobenzothiophen-5-yl)-N-methyl-carbamate (2) (0.365 g, 1.21 mmol, 35.93% yield) was obtained as a colorless oil.

### Procedure for preparation of 2-chloro-N-methyl-4,5,6,7-tetrahydrobenzothiophen-5-amine hydrochloride (97)

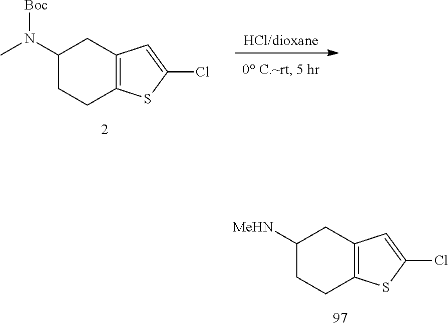 [SMILES: CN(C(=O)OC(C)(C)C)C1CCc2sc(Cl)cc2C1 ; "HCl/dioxane" ; CNC1CCc2sc(Cl)cc2C1]

A mixture of tert-butyl N-(2-chloro-4,5,6,7-tetrahydrobenzothiophen-5-yl)-N-methyl-carbamate (2) (66 mg, 218.67 μmol, 1 eq), HCl/dioxane (4 M, 1 mL, 18.29 eq) in dioxane (1 mL) was stirred at 0-25° C. for 5 hr under N₂ atmosphere. The mixture was then concentrated under reduced pressure. The crude product was triturated with acetonitrile at 25° C. for 2 min filtered and collected. Compound 2-chloro-N-methyl-4,5,6,7-tetrahydrobenzothiophen-5-amine hydrochloride (97) (35.95 mg, 147.92 μmol, 67.65% yield) was obtained as a white solid. ¹H NMR (400 MHz, METHANOL-d4) δ=6.71 (s, 1H), 3.57-3.45 (m, 1H), 3.31 (s, 14H), 3.10 (dd, J=5.3, 15.9 Hz, 1H), 2.90-2.85 (m, 2H), 2.78 (s, 3H), 2.70-2.57 (m, 1H), 2.42-2.25 (m, 1H), 2.05-1.86 (m, 1H). LCMS: m/z [M+H]⁺=202.0

### Synthetic scheme for preparation of 2-fluoro-N-methyl-4,5,6,7-tetrahydrobenzothiophen-5-amine hydrochloride enantiomer 1 (98) and enantiomer 2 (99)

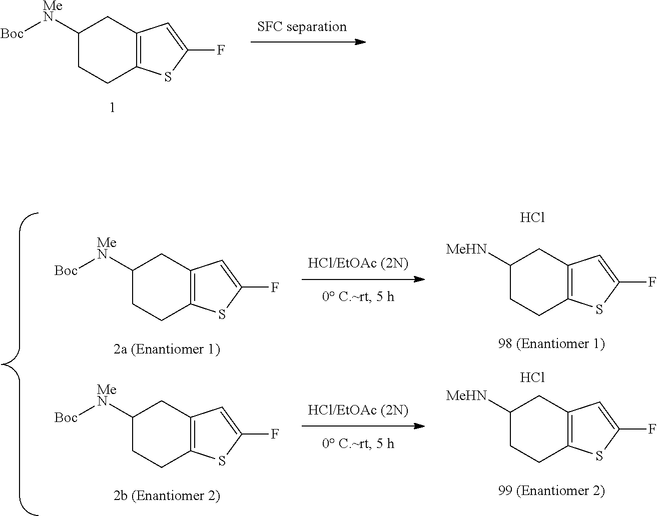

### Procedure for preparation of tert-butyl N-(2-fluoro-4,5,6,7-tetrahydrobenzothiophen-5-yl)-N-methyl-carbamate enantiomer 1 (2a) and enantiomer 2 (2b)

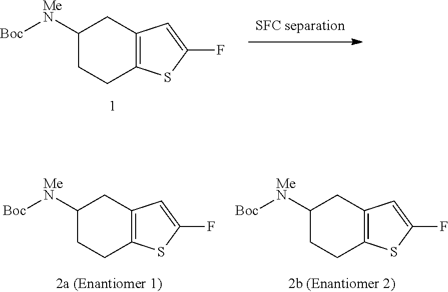 [SMILES: CN(C(=O)OC(C)(C)C)C1CCc2sc(F)cc2C1 ; CN(C(=O)OC(C)(C)C)C1CCc2sc(F)cc2C1 ; CN(C(=O)OC(C)(C)C)C1CCc2sc(F)cc2C1]

Racemic tert-butyl N-(2-fluoro-4,5,6,7-tetrahydrobenzothiophen-5-yl)-N-methyl-carbamate (1) was separated to its enantiomers by SFC (column: DAICEL CHIRALPAK IG (250 mm*50 mm, 10 μm); mobile phase: [CO₂-MeOH (0.1% NH₃H₂O)]; 10% B isocratic elution mode) to yield tert-butyl N-(2-fluoro-4,5,6,7-tetrahydrobenzothiophen-5-yl)-N-methyl-carbamate enantiomer 1 (2a), (210 mg) as yellow oil and tert-butyl N-(2-fluoro-4,5,6,7-tetrahydrobenzothiophen-5-yl)-N-methyl-carbamate enantiomer 2 (2b), (210 mg) as yellow oil.

### Procedure for preparation of 2-fluoro-N-methyl-4,5,6,7-tetrahydrobenzothiophen-5-amine hydrochloride enantiomer 1 (98)

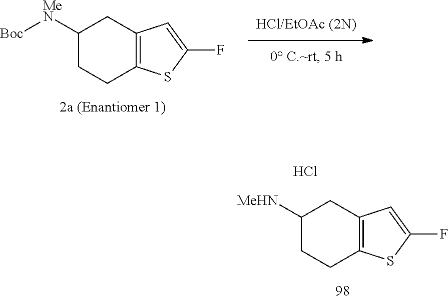 [SMILES: CN(C(=O)OC(C)(C)C)C1CCc2sc(F)cc2C1 ; Cl ; CNC1CCc2sc(F)cc2C1]

To a solution of tert-butyl N-(2-fluoro-4,5,6,7-tetrahydrobenzothiophen-5-yl)-N-methyl-carbamate enantiomer 1 (2a) (210 mg, 735.87 μmol, 1 eq) in EtOAc (2 mL) was added HCl/EtOAc (4 M, 2 mL, 10.87 eq) at 0° C. The mixture was stirred at 0~25° C. for 5 hr and concentrated and the residue was triturated with acetonitrile (2 mL) at 25° C. for 2 min. Compound 2-fluoro-N-methyl-4,5,6,7-tetrahydrobenzothiophen-5-amine hydrochloride (98) (113.92 mg, 608.77 μmol, 82.73% yield) was obtained as a white solid. ¹H NMR (400 MHz, METHANOL-d4) δ=6.28 (d, J=2.0 Hz, 1H), 3.53-3.44 (m, 1H), 3.04 (dd, J=4.9, 15.7 Hz, 1H), 2.85-2.80 (m, 2H), 2.78 (s, 3H), 2.60 (br dd, J=9.1, 15.8 Hz, 1H), 2.37-2.27 (m, 1H), 1.94 (tdd, J=7.8, 10.8, 12.8 Hz, 1H). LCMS: m/z [M+H]⁺=186.0

### Procedure for preparation of 2-fluoro-N-methyl-4,5,6,7-tetrahydrobenzothiophen-5-amine hydrochloride enantiomer 2 (99)

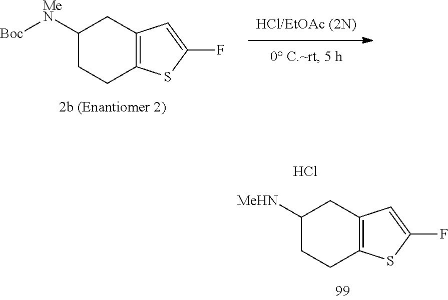 [SMILES: CN(C(=O)OC(C)(C)C)C1CCc2sc(F)cc2C1 ; Cl ; CNC1CCc2sc(F)cc2C1]

To a solution of tert-butyl N-(2-fluoro-4,5,6,7-tetrahydrobenzothiophen-5-yl)-N-methyl-carbamate enantiomer 2 (2b) (210 mg, 735.87 μmol, 1 eq) in EtOAc (2 mL) was added HCl/EtOAc (4 M, 2 mL, 10.87 eq) at 0° C. The mixture was stirred at 0~25° C. for 5hrand then concentrated to afford the crude product. The crude product was triturated with acetonitrile (2 mL) at 25° C. for 2 min. Compound 2-fluoro-N-methyl-4,5,6,7-tetrahydrobenzothiophen-5-amine hydrochloride (99) (122.90 mg, 656.75 μmol, 89.25% yield) was obtained as a white solid. ¹H NMR (400 MHz, METHANOL-d4) δ=6.27 (d, J=2.0 Hz, 1H), 3.53-3.44 (m, 1H), 3.11-3.00 (m, 1H), 2.85-2.79 (m, 2H), 2.77 (s, 3H), 2.60 (br dd, J=9.1, 15.8 Hz, 1H), 2.37-2.27 (m, 1H), 1.94 (tdd, J=7.8, 10.8, 12.8 Hz, 1H). LCMS: m/z [M+H]⁺=186.0

### Synthetic scheme for preparation of 1-fluoro-N, 3-dimethyl-4,5,6,7-tetrahydro-2-benzothiophen-5-amine hydrochloride (100)

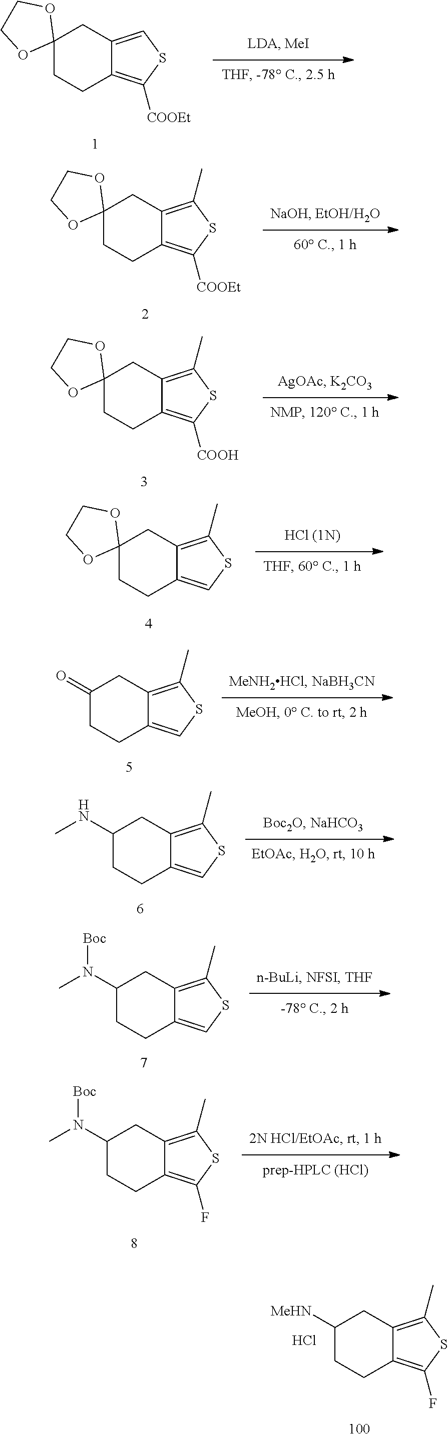

### Procedure for preparation of ethyl 3′-methylspiro[1,3-dioxolane-2,5′-6,7-dihydro-4H-2-benzothiophene]-1′-carboxylate (2)

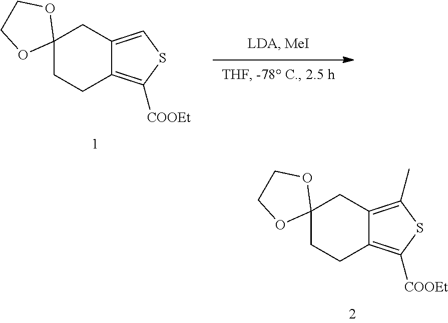 [SMILES: CCOC(=O)c1scc2c1CCC1(C2)OCCO1 ; CCOC(=O)c1sc(C)c2c1CCC1(C2)OCCO1]

To a solution of ethyl spiro[1,3-dioxolane-2,5′-6,7-dihydro-4H-2-benzothiophene]-1′-carboxylate (1) (10 g, 37.3 mmol, 1 eq) in THF (150 mL) was added dropwise LDA (2 M, 27.9 mL, 1.5 eq) at −78° C. After addition, the mixture was stirred at −78° C. for 0.5 hr, and then MeI (7.93 g, 55.9 mmol, 3.48 mL, 1.5 eq) was added dropwise at −78° C. The resulting mixture was stirred at −78° C. for 2 hr. The reaction mixture was diluted with sat. aq. NH₄Cl 100 mL and extracted with EtOAc (100 mL*2). The combined organic layers were washed with sat. aq. NaCl (100 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~15% Ethyl acetate/Petroleum ether gradient @40 mL/min). Compound ethyl 3′-methylspiro[1,3-dioxolane-2,5′-6,7-dihydro-4H-2-benzothiophene]-1′-carboxylate (2) (9.9 g, 35.06 mmol, 94.08% yield) was obtained as a yellow oil. ¹H NMR (400 MHz, CHLOROFORM-d) δ=4.29 (q, J=7.1 Hz, 2H), 4.06-3.99 (m, 4H), 3.19 (t, J=6.8 Hz, 2H), 2.74 (s, 2H), 2.32 (s, 3H), 1.92 (t, J=6.8 Hz, 2H), 1.34 (t, J=7.1 Hz, 3H) LCMS: m/z [M+1]⁺=283.1

### Procedure for preparation of 3′-methylspiro[1,3-dioxolane-2,5′-6,7-dihydro-4H-2-benzothiophene]-1′-carboxylic acid (3)

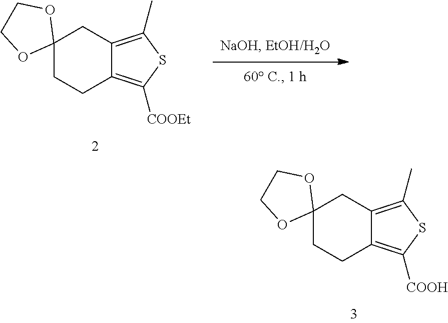 [SMILES: CCOC(=O)c1sc(C)c2c1CCC1(C2)OCCO1 ; Cc1sc(C(=O)O)c2c1CC1(CC2)OCCO1]

To a solution of ethyl 3′-methylspiro[1,3-dioxolane-2,5′-6,7-dihydro-4H-2-benzothiophene]-1′-carboxylate (2) (9.9 g, 35.1 mmol, 1 eq) in EtOH (50 mL) and H₂O (50 mL) was added NaOH (5.61 g, 140 mmol, 4 eq). The mixture was stirred at 60° C. for 1 hr. The reaction mixture was concentrated under vacuum to remove EtOH. The aqueous phase was diluted with water (20 mL), extracted with EtOAc (50 mL). The separated aqueous phase pH was adjusted with citric acid to 3-4 and then extracted with EtOAc (50 mL*3). The combined organic phases were washed with brine (30 mL), dried over anhydrous sodium sulfate, filtered and concentrated under vacuum to give the product. 3′-methylspiro[1,3-dioxolane-2,5′-6,7-dihydro-4H-2-benzothiophene]-1′-carboxylic acid (3) (8.2 g, 32.25 mmol) was obtained as a white solid. ¹H NMR (400 MHz, CHLOROFORM-d) δ=4.05 (s, 4H), 3.21 (t, J=6.8 Hz, 2H), 2.76 (s, 2H), 2.35 (s, 3H), 1.93 (t, J=6.7 Hz, 2H) LCMS: m/z [M+H]⁺=255.2

### Procedure for preparation of 1′-methylspiro[1,3-dioxolane-2,6′-5,7-dihydro-4H-2-benzothiophene](4)

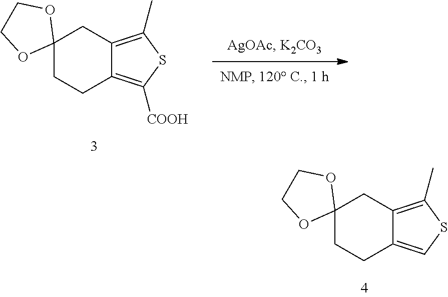 [SMILES: Cc1sc(C(=O)O)c2c1CC1(CC2)OCCO1 ; Cc1scc2c1CC1(CC2)OCCO1]

To a solution of 3′-methylspiro[1,3-dioxolane-2,5′-6,7-dihydro-4H-2-benzothiophene]-1′-carboxylic acid (3) (8.2 g, 32.25 mmol, 1 eq) in NMP (80 mL) was added AgOAc (889 mg, 3.22 mmol, 146.31 μL, 0.1 eq) and K₂CO₃ (13.4 g, 96.7 mmol, 3 eq). The mixture was stirred at 120° C. for 1 hr. To the reaction mixture was added H₂O 100 mL, and then extracted with EtOAc (300 mL*2), the combined organic layers were washed with brine (100 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~20% Ethyl acetate/Petroleum ether gradient @40 mL/min). The crude product 1′-methylspiro[1,3-dioxolane-2,6′-5,7-dihydro-4H-2-benzothiophene](4) (6 g, 28.53 mmol, 88.48% yield) was obtained as a white solid. ¹H NMR (400 MHz, CHLOROFORM-d) δ=6.67 (s, 1H), 4.08-4.00 (m, 4H), 2.86 (t, J=6.4 Hz, 2H), 2.77 (s, 2H), 2.30 (s, 3H), 1.91 (t, J=6.7 Hz, 2H). LCMS: m/z [M+H]⁺=211.1

### Procedure for preparation of 3-methyl-6,7-dihydro-4H-2-benzothiophen-5-one (5)

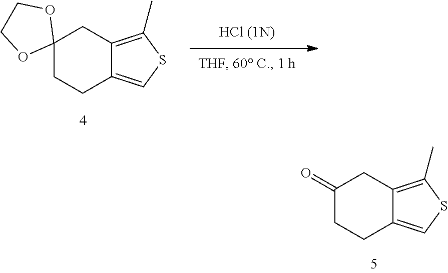

To a solution of 1′-methylspiro[1,3-dioxolane-2,6′-5,7-dihydro-4H-2-benzothiophene](4) (6 g, 28.5 mmol, 1 eq) in THF (100 mL) and aq HCl (100 mL). The mixture was stirred at 60° C. for 1 hr. The reaction mixture was quenched by addition sat. aq. NaHCO₃ (100 mL) at 0° C., and then extracted with EtOAc (50 mL*3). The combined organic layers were washed with brine (50 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~20% Ethyl acetate/Petroleum ether gradient @40 mL/min). Compound 3-methyl-6,7-dihydro-4H-2-benzothiophen-5-one (5) (4.3 g, 25.87 mmol, 90.66% yield) was obtained as a yellow oil. ¹H NMR (400 MHz, CHLOROFORM-d) δ=6.78 (s, 1H), 3.43 (s, 2H), 2.97 (t, J=6.8 Hz, 2H), 2.61-2.54 (m, 2H), 2.33 (s, 3H). LCMS: m/z [M+H]⁺=167.0

### Procedure for preparation of N, 3-dimethyl-4,5,6,7-tetrahydro-2-benzothiophen-5-amine (6)

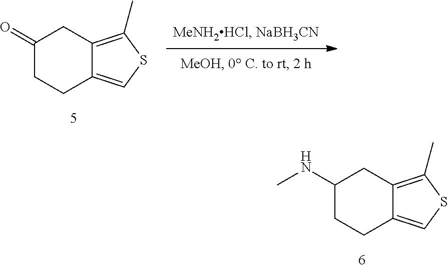

To the mixture of 3-methyl-6,7-dihydro-4H-2-benzothiophen-5-one (5) (2 g, 12.03 mmol, 1 eq) and methylamine hydrochloride (3.25 g, 48.12 mmol, 4 eq) in MeOH (50 mL) was added sodium cyanoborohydride (4.54 g, 72.18 mmol, 6 eq) at 0° C. The resulting mixture was stirred at 25° C. for 2 hr. Remove the solvent on a rotary evaporator to afford crude N, 3-dimethyl-4,5,6,7-tetrahydro-2-benzothiophen-5-amine (6) (2 g, crude). LCMS: m/z [M+H]⁺=182.2

### Procedure for preparation of tert-butyl N-methyl-N-(3-methyl-4,5,6,7-tetrahydro-2-benzothiophen-5-yl)carbamate (7)

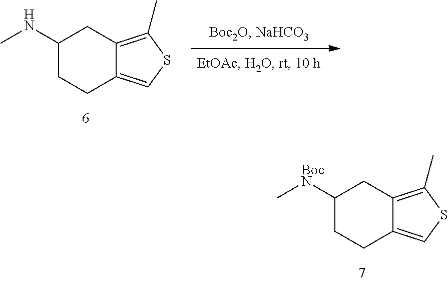 [SMILES: CNC1CCc2csc(C)c2C1 ; Cc1scc2c1CC(N(C)C(=O)OC(C)(C)C)CC2]

To a solution of N, 3-dimethyl-4,5,6,7-tetrahydro-2-benzothiophen-5-amine (6) (2 g, 11.03 mmol, 1 eq) in H₂O (10 mL) and EtOAc (10 mL) was added NaHCO₃ (4.63 g, 55.16 mmol, 2.15 mL, 5 eq) and (Boc)₂O (4.82 g, 22.06 mmol, 5.07 mL, 2 eq). The mixture was stirred at 25° C. for 10 hr. The reaction mixture was poured into H₂O (10 mL) and extracted with EtOAc (30 mL*2). The combined organic phases were washed with brine (10 mL), dried over anhydrous sodium sulfate, filtered and concentrated under vacuum to give a residue. The residue was purified by flash silica gel chromatography (ISCO®; 20 g SepaFlash® Silica Flash Column, eluent 0~20% Ethyl acetate/Petroleum ether gradient @40 mL/min) to give tert-butyl N-methyl-N-(3-methyl-4,5,6,7-tetrahydro-2-benzothiophen-5-yl)carbamate (7) (2 g, 7.11 mmol, 64.42% yield) as a yellow oil. ¹H NMR (400 MHz, CHLOROFORM-d) δ=6.64 (s, 1H), 4.42-4.24 (m, 1H), 2.97-2.65 (m, 6H), 2.49 (br dd, J=11.9, 15.2 Hz, 1H), 2.31 (s, 3H), 1.91-1.72 (m, 2H), 1.49 (s, 9H)

### Procedure for preparation of tert-butyl N-(1-fluoro-3-methyl-4,5,6,7-tetrahydro-2-benzothiophen-5-yl)-N-methyl-carbamate (8)

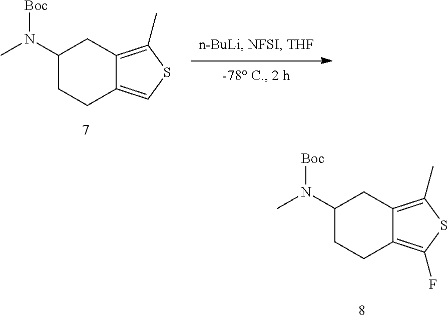 [SMILES: Cc1scc2c1CC(N(C)C(=O)OC(C)(C)C)CC2 ; Cc1sc(F)c2c1CC(N(C)C(=O)OC(C)(C)C)CC2]

To a solution of tert-butyl N-methyl-N-(3-methyl-4,5,6,7-tetrahydro-2-benzothiophen-5-yl)carbamate (7) (500 mg, 1.78 mmol, 1 eq) in THF (10 mL) was added n-BuLi (2.5 M, 1.07 mL, 1.5 eq) dropwise at −78° C. After addition, the mixture was stirred at −78° C. for 0.5 hr, and then NFSI (840.42 mg, 2.67 mmol, 1.5 eq) in THF (10 mL) was added dropwise at −78° C. The resulting mixture was stirred at −78° C. for 1.5 hr. The reaction mixture was quenched by addition aq. sat. NH₄Cl 20 mL and extracted with EtOAc (30 mL*3). The combined organic layers were washed with brine (30 mL), dried over anhydrous Na₂SO₄, filtered and concentrated under reduced pressure. The residue was purified by prep-HPLC (column: Phenomenex luna C18 150*25 mm*10 μm; mobile phase: [water(FA)-ACN]; gradient:70%-100% B over 10 min) to give tert-butyl N-(1-fluoro-3-methyl-4,5,6,7-tetrahydro-2-benzothiophen-5-yl)-N-methyl-carbamate (8) (200 mg, 668.00 μmol, 37.60% yield) as a yellow oil. ¹H NMR (400 MHz, METHANOL-d₄) δ=4.23-4.09 (m, 1H), 2.91-2.77 (m, 4H), 2.67 (br dd, J=4.9, 15.6 Hz, 1H), 2.56-2.43 (m, 2H), 2.19 (d, J=2.1 Hz, 3H), 1.87-1.75 (m, 2H), 1.48 (s, 9H)

### Procedure for preparation of 1-fluoro-N, 3-dimethyl-4,5,6,7-tetrahydro-2-benzothiophen-5-amine hydrochloride (100)

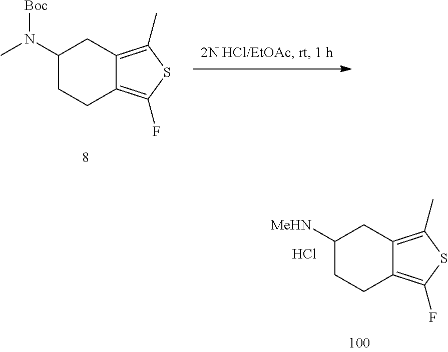 [SMILES: Cc1sc(F)c2c1CC(N(C)C(=O)OC(C)(C)C)CC2 ; Cl ; CNC1CCc2c(F)sc(C)c2C1]

To a solution of tert-butyl N-(1-fluoro-3-methyl-4,5,6,7-tetrahydro-2-benzothiophen-5-yl)-N-methyl-carbamate (8) (200 mg, 668.00 μmol, 1 eq) in EtOAc (2 mL) was added HCl/EtOAc (4 M, 2 mL, 11.98 eq). The mixture was stirred at 25° C. for 1 hr. The reaction mixture was concentrated under vacuum to remove the solvent. The residue was purified by prep-HPLC (column: Welch Xtimate C18 150*25 mm*5 μm; mobile phase: [water(HCl)-ACN]; gradient:5%-35% B over 9 min) to give 1-fluoro-N, 3-dimethyl-4,5,6,7-tetrahydro-2-benzothiophen-5-amine hydrochloride (100) (72.41 mg, 307.15 μmol, 40.81% yield) as a white solid. ¹H NMR (400 MHz, METHANOL-d₄) δ=3.41 (ddt, J=2.8, 5.3, 10.3 Hz, 1H), 3.12 (dd, J=4.9, 16.1 Hz, 1H), 2.90-2.82 (m, 1H), 2.79 (s, 3H), 2.66-2.45 (m, 2H), 2.27-2.16 (m, 4H), 1.77 (dtd, J=5.5, 10.9, 12.8 Hz, 1H). LCMS: m/z [M+H]⁺=200.1

### Synthetic scheme for preparation of N, 5-dimethyl-6,7-dihydro-4H-2-benzothiophen-5-amine hydrochloride enantiomer1 (101), and enantiomer 2 (102)

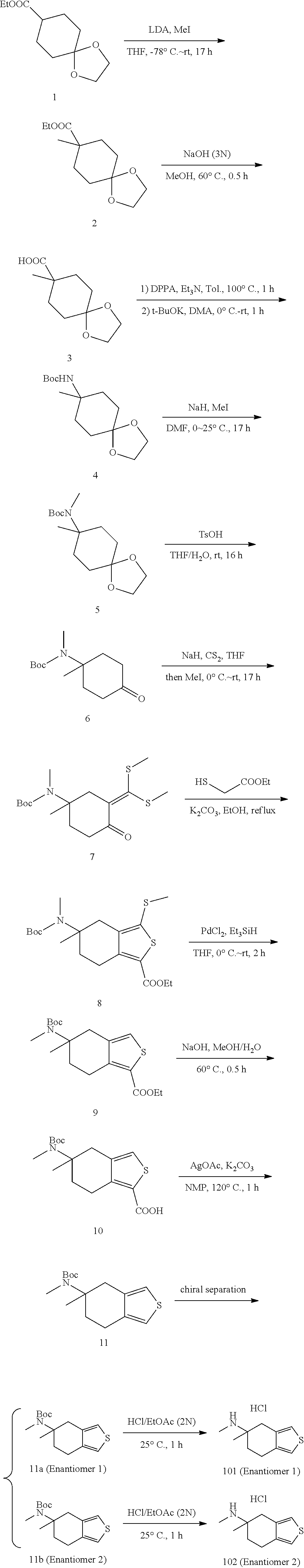

### Procedure for preparation of ethyl 8-methyl-1,4-dioxaspiro[4.5]decane-8-carboxylate (2)

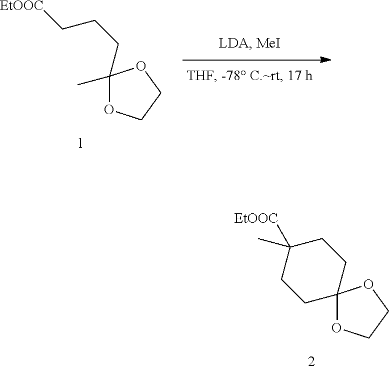 [SMILES: CCOC(=O)CCCC1(C)OCCO1 ; "LDA, MeI" ; CCOC(=O)C1(C)CCC2(CC1)OCCO2]

To a solution of LDA (2 M, 206.29 mL, 1.3 eq) in THF (500 mL) was added ethyl 1,4-dioxaspiro[4.5]decane-8-carboxylate (1) (68 g, 317.38 mmol, 1 eq) in THF (200 mL) dropwise at −78° C. for 1 h under N₂, then was added iodomethane (76.58 g, 539.54 mmol, 33.59 mL, 1.7 eq) dropwise at −78° C. The mixture was stirred at −78° C. for 1 h, then was warmed to 25° C. and stirred for 16 hr. The mixture was treated with NH₄Cl (aq, 300 mL) and extracted with EtOAc (500 mL*3). The combined organic phase was washed with brine (200 mL*3), dried over Na₂SO₄, filtered and concentrated at reduced pressure. The residue was purified by silica gel chromatography (100-200 mesh silica gel, Petroleum ether/Ethyl acetate=1/0~10/1, Rf=0.25) to afford ethyl 8-methyl-1,4-dioxaspiro[4.5]decane-8-carboxylate (2) (66.9 g, 293.06 mmol, 92.34% yield) as a white solid. ¹H NMR (400 MHz, METHANOL-d₄) δ 4.15 (q, J=7.0 Hz, 2H), 3.91 (s, 4H), 2.13-2.06 (m, 2H), 1.68-1.61 (m, 2H), 1.59-1.45 (m, 4H), 1.25 (t, J=7.1 Hz, 3H), 1.17 (s, 3H).

### Procedure for preparation of 8-methyl-1,4-dioxaspiro[4.5]decane-8-carboxylic acid (3)

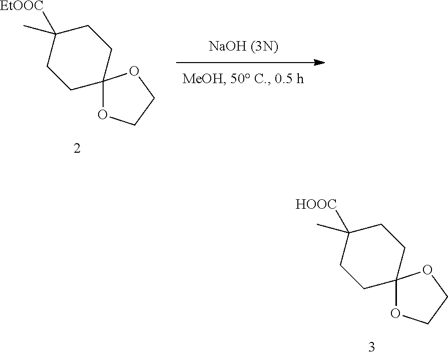 [SMILES: CCOC(=O)C1(C)CCC2(CC1)OCCO2 ; "NaOH (3N)" ; CC1(C(=O)O)CCC2(CC1)OCCO2]

To a solution of ethyl 8-methyl-1,4-dioxaspiro[4.5]decane-8-carboxylate (2) (66.9 g, 293.06 mmol, 1 eq) in MeOH (400 mL) was added NaOH (3 M, 293.06 mL, 3 eq). The mixture was stirred at 60° C. for 0.5 hr. The mixture was concentrated at reduced pressure to give a residue and acidified to pH=7 with aq. HCl (1 M) and then to pH=5 with citric acid (aq. 100 mL). The mixture was extracted with EtOAc (500 mL*3). The combined organic phase was washed with brine (200 mL*3), dried over Na₂SO₄, filtered and concentrated at reduced pressure. The crude product 8-methyl-1,4-dioxaspiro[4.5]decane-8-carboxylic acid (3) (58 g, crude) was obtained as a white solid and used into the next step without further purification. ¹H NMR (400 MHz, METHANOL-d₄) δ 3.93 (s, 4H), 2.16-2.05 (m, 2H), 1.71-1.60 (m, 4H), 1.56-1.47 (m, 2H), 1.21 (s, 3H)

### Procedure for preparation of tert-butyl N-(8-methyl-1,4-dioxaspiro[4.5]decan-8-yl) carbamate (4)

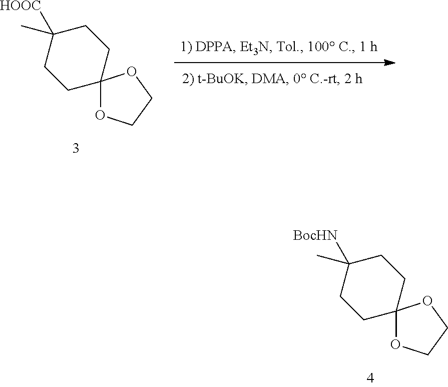

To a solution of 8-methyl-1,4-dioxaspiro[4.5]decane-8-carboxylic acid (3) (58 g, 289.67 mmol, 1 eq) in Tol. (500 mL) was added DPPA (95.66 g, 347.60 mmol, 75.32 mL, 1.2 eq) and TEA (35.17 g, 347.60 mmol, 48.38 mL, 1.2 eq). The mixture was stirred at 100° C. for 1 hr. The mixture was cooled on an ice bath, sat. NaHCO₃ (200 mL) was added to the mixture, extracted with EtOAc (200 mL*2). The combined organic layers were washed with brine (100 mL), dried over anhydrous Na₂SO₄, filtered and concentrated under reduced pressure. The residue was dissolved in DMA (300 mL) was added tBuOK (39.00 g, 347.60 mmol, 1.2 eq) at 0° C., then was stirred at 20° C. for 1 hr. The mixture was quenched with NH₄Cl (aq, 300 mL) and extracted with EtOAc (500 mL*3). The combined organic phase was washed with brine (200 mL*3), dried over Na₂SO₄, filtered and concentrated at reduced pressure to give a residue. The residue was purified by silica gel chromatography (100-200 mesh silica gel, Petroleum ether/Ethyl acetate=1/0~10/1, Rf=0.54) to afford tert-butyl N-(8-methyl-1,4-dioxaspiro[4.5]decan-8-yl)carbamate (4) (70 g, 257.97 mmol, 89.06% yield) as a white solid. ¹H NMR (400 MHz, METHANOL-d₄) δ 3.91 (s, 4H), 2.07 (br d, J=11.9 Hz, 2H), 1.73-1.62 (m, 2H), 1.58-1.50 (m, 4H), 1.43 (s, 9H), 1.27 (s, 3H).

### Procedure for preparation of tert-butyl N-methyl-N-(8-methyl-1,4-dioxaspiro[4.5]decan-8-yl)carbamate (5)

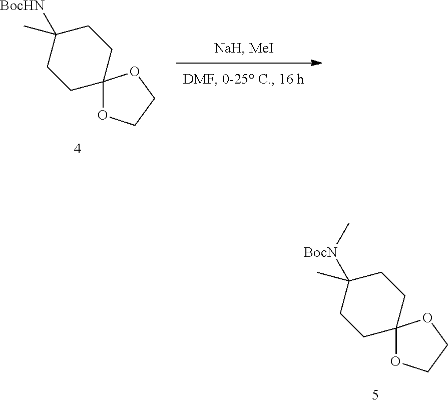

To a mixture of tert-butyl N-(8-methyl-1,4-dioxaspiro[4.5]decan-8-yl)carbamate (4) (25 g, 92.13 mmol, 1 eq) and MeI (19.62 g, 138.20 mmol, 8.60 mL, 1.5 eq) in DMF (500 mL) was added NaH (4.42 g, 110.56 mmol, 60% suspension in mineral oil, 1.2 eq) in one portion at 0° C. under N₂. The mixture was stirred at 0° C. for 1 hour. Then to the mixture was added MeI (19.62 g, 138.20 mmol, 8.60 mL, 1.5 eq) at 0° C. The mixture was stirred at 25° C. for 16 hours. The mixture was quenched with saturated ammonium chloride aqueous solution (50 mL). The mixture was extracted with EtOAc (50 mL*3). The combined organic phases were washed with brine (50 mL*3), dried over Na₂SO₄, filtered and concentrated at reduced pressure. The residue was purified by silica gel chromatography (100-200 mesh silica gel, Petroleum ether/Ethyl acetate=1/0~1/1, Rf=0.26) to afford tert-butyl N-methyl-N-(8-methyl-1,4-dioxaspiro[4.5]decan-8-yl)carbamate (5) (24 g, 84.10 mmol, 91.28% yield) as a white solid. ¹H NMR (400 MHz, METHANOL-d₄) δ 3.92 (s, 4H), 2.86 (s, 3H), 2.50-2.38 (m, 2H), 1.68-1.55 (m, 6H), 1.45 (s, 9H), 1.22 (s, 3H). LCMS: m/z [M+H-Boc]⁺=186.2

### Procedure for preparation of tert-butyl N-methyl-N-(1-methyl-4-oxo-cyclohexyl)carbamate (6)

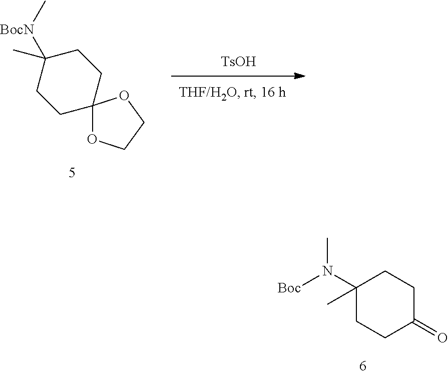 [SMILES: CN(C(=O)OC(C)(C)C)C1(C)CCC2(CC1)OCCO2 ; "TsOH" ; CN(C(=O)OC(C)(C)C)C1(C)CCC(=O)CC1]

To a mixture of tert-butyl N-methyl-N-(8-methyl-1,4-dioxaspiro[4.5]decan-8-yl)carbamate (5) (24 g, 84.10 mmol, 1 eq) in THF (100 mL) and H₂O (100 mL) was added TsOH (28.96 g, 168.20 mmol, 2 eq) under N₂. The mixture was stirred at 25° C. for 16 hours. The reaction mixture was quenched by addition NaHCO₃ (aq.) to adjust pH=7, and then diluted with H₂O (50 mL) and extracted with EtOAc (100 mL*3). The combined organic layers were washed with brine (30 mL), dried over anhydrous Na₂SO₄, filtered and concentrated under reduced pressure. The residue was purified by silica gel chromatography (100-200 mesh silica gel, Petroleum ether/Ethyl acetate=1/0~3/1, Rf=0.54) to afford tert-butyl N-methyl-N-(1-methyl-4-oxo-cyclohexyl)carbamate (6) (7.58 g, 31.41 mmol, 37.35% yield) as a white solid. ¹H NMR (400 MHz, METHANOL-d₄) δ 2.95 (s, 3H), 2.82-2.71 (m, 2H), 2.44-2.33 (m, 2H), 2.30-2.21 (m, 2H), 1.80 (dt, J=4.9, 13.1 Hz, 2H), 1.47 (s, 9H), 1.33 (s, 3H)

### Procedure for preparation of tert-butyl N-[3-[bis(methylsulfanyl)methylene]-1-methyl-4-oxo-cyclohexyl]-N-methyl-carbamate (7)

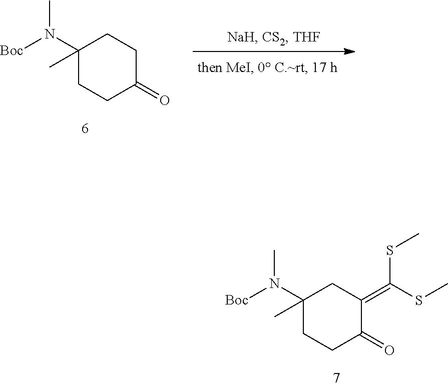

To a solution of tert-butyl N-methyl-N-(1-methyl-4-oxo-cyclohexyl)carbamate (6) (7.58 g, 31.41 mmol, 1 eq) in THF (150 mL) was added NaH (1.38 g, 34.55 mmol, 60% suspension in mineral oil, 1.1 eq) at 0° C. stirred for 0.5 h, then was added CS₂ (2.87 g, 37.69 mmol, 2.28 mL, 1.2 eq). The mixture was stirred at 0° C. for 0.5 h. The mixture was added MeI (13.37 g, 94.23 mmol, 5.87 mL, 3 eq) at 0° C. and stirred at 25° C. for 16 hr. The reaction mixture was quenched by addition NH₄Cl (aq, 100 mL), and then diluted with H₂O 100 mL and extracted with EtOAc (100 mL*3). The combined organic layers were washed with brine (100 mL), dried over anhydrous Na₂SO₄, filtered and concentrated under reduced pressure. The residue was purified by silica gel chromatography (100-200 mesh silica gel, Petroleum ether/Ethyl acetate=1/0~511, Rf=0.37) to afford tert-butyl N-[3-[bis(methylsulfanyl)methylene]-1-methyl-4-oxo-cyclohexyl]-N-methyl-carbamate (7) (9 g, 26.05 mmol, 82.93% yield) as a white solid. ¹H NMR (400 MHz, METHANOL-d₄) δ 2.95 (s, 3H), 2.91-2.83 (m, 2H), 2.42 (s, 3H), 2.40 (s, 3H), 2.30-2.22 (m, 2H), 1.80 (dt, J=4.6, 13.1 Hz, 2H), 1.47 (s, 9H), 1.33 (s, 3H). LCMS: m/z [M+H-tBu]⁺=290.1.

### Procedure for preparation of ethyl 5-[tert-butoxycarbonyl(methyl)amino]-5-methyl-3-methylsulfanyl-6,7-dihydro-4H-2-benzothiophene-1-carboxylate (8)

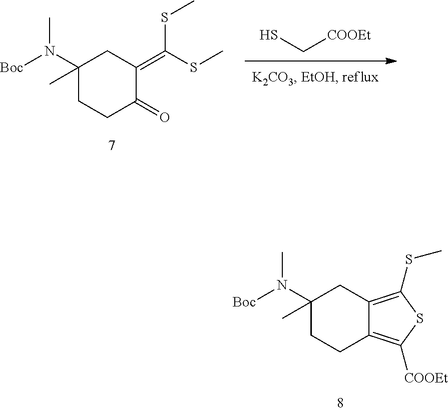 [SMILES: CSC(SC)=C1CC(C)(N(C)C(=O)OC(C)(C)C)CCC1=O ; CCOC(=O)CS ; CCOC(=O)c1sc(SC)c2c1CCC(C)(N(C)C(=O)OC(C)(C)C)C2]

To a mixture of tert-butyl N-[3-[bis(methylsulfanyl)methylene]-1-methyl-4-oxo-cyclohexyl]-N-methyl-carbamate (7) (9 g, 26.05 mmol, 1 eq) and K₂CO₃ (4.32 g, 31.26 mmol, 1.2 eq) in EtOH (100 mL) was added ethyl 2-sulfanylacetate (4.03 g, 33.54 mmol, 3.66 mL, 1.29 eq) under N₂. The mixture was stirred at 80° C. for 16 hours. The mixture was extracted with EtOAc (100 mL*3). The combined organic phases were washed with brine (100 mL*3), dried over Na₂SO₄, filtered and concentrated at reduced pressure. The residue was purified by silica gel chromatography (100-200 mesh silica gel, Petroleum ether/Ethyl acetate=1/0~5/1, Rf=0.48) to afford ethyl 5-[tert-butoxycarbonyl(methyl)amino]-5-methyl-3-methylsulfanyl-6,7-dihydro-4H-2-benzothiophene-1-carboxylate (8) (7.8 g, 19.52 mmol, 74.94% yield) as a white solid. ¹H NMR (400 MHz, METHANOL-d₄) δ 4.19 (q, J=7.1 Hz, 2H), 2.95 (s, 3H), 2.79-2.73 (m, 2H), 2.37 (br dd, J=5.0, 12.1 Hz, 2H), 2.30-2.23 (m, 2H), 1.80 (dt, J=4.8, 13.2 Hz, 2H), 1.47 (s, 9H), 1.32 (s, 3H), 1.28 (t, J=7.1 Hz, 3H). LCMS: m/z [M+H-Boc]⁺=300.1

### Procedure for preparation of ethyl 5-[tert-butoxycarbonyl(methyl)amino]-5-methyl-6,7-dihydro-4H-2-benzothiophene-1-carboxylate (9)

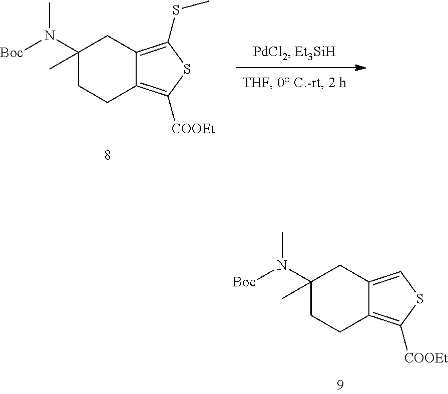 [SMILES: CCOC(=O)c1sc(SC)c2c1CCC(C)(N(C)C(=O)OC(C)(C)C)C2 ; CCOC(=O)c1scc2c1CCC(C)(N(C)C(=O)OC(C)(C)C)C2]

To a mixture of ethyl 5-[tert-butoxycarbonyl(methyl)amino]-5-methyl-3-methylsulfanyl-6,7-dihydro-4H-2-benzothiophene-1-carboxylate (8) (7.8 g, 19.52 mmol, 1 eq) and PdCl₂ (346.16 mg, 1.95 mmol, 0.1 eq) in THF (80 mL) was added triethylsilane (4.99 g, 42.95 mmol, 6.86 mL, 2.2 eq) at 0° C. under N₂. The mixture was stirred at 25° C. for 2 hours. The reaction mixture was quenched by addition H₂O (50 mL). The mixture was extracted with EtOAc (50 mL*3). The combined organic phases were washed with brine (50 mL*3), dried over Na₂SO₄, filtered and concentrated at reduced pressure. The residue was purified by silica gel chromatography (100-200 mesh silica gel, Petroleum ether/Ethyl acetate=1/0~5/1, Rf=0.50) to afford ethyl 5-[tert-butoxycarbonyl(methyl)amino]-5-methyl-6,7-dihydro-4H-2-benzothiophene-1-carboxylate (9) (4.5 g, 12.73 mmol, 65.22% yield) as a white solid. ¹H NMR (400 MHz, Methanol-d4, 298 K) δ 7.28 (s, 1H), 4.29 (q, J=7.1 Hz, 2H), 3.18-3.09 (m, 1H), 2.93 (tdd, J=2.9, 5.8, 13.6 Hz, 1H), 2.82-2.73 (m, 1H), 2.72 (s, 3H), 2.67 (d, J=16.8 Hz, 1H), 1.77-1.59 (m, 2H), 1.45 (s, 9H), 1.40 (s, 3H), 1.34 (t, J=7.1 Hz, 3H). LCMS: m/z [M+H-Boc]⁺=254.1.

### Procedure for preparation of 5-[tert-butoxycarbonyl (methyl)amino]-5-methyl-6,7-dihydro-4H-2-benzothiophene-1-carboxylic acid (10)

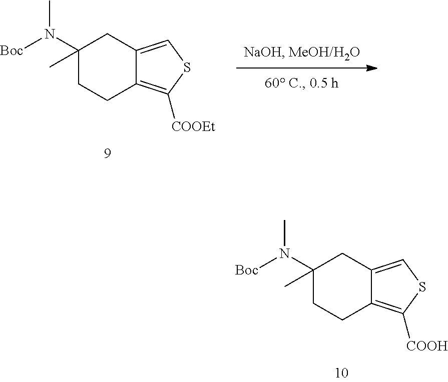 [SMILES: CCOC(=O)c1scc2c1CCC(C)(N(C)C(=O)OC(C)(C)C)C2 ; CN(C(=O)OC(C)(C)C)C1(C)CCc2c(csc2C(=O)O)C1]

To a solution of ethyl 5-[tert-butoxycarbonyl(methyl)amino]-5-methyl-6,7-dihydro-4H-2-benzothiophene-1-carboxylate (9) (4.5 g, 12.73 mmol, 1 eq) in MeOH (40 mL) was added NaOH (3 M, 12.73 mL, 3 eq). The mixture was stirred at 60° C. for 0.5 hr. The mixture was concentrated at reduced pressure to give a residue and acidified to pH=7 by HCl (1 M) and then acidified to pH=5 by citric acid (aq., 50 mL). The mixture was extracted with EtOAc (100 mL*3). The combined organic phases were washed with brine (100 mL*3), dried over Na₂SO₄, filtered and concentrated at reduced pressure. The crude product was triturated with Petroleum ether/Ethyl acetate=10/1 at 25° C. for 30 min. Compound 5-[tert-butoxycarbonyl(methyl)amino]-5-methyl-6,7-dihydro-4H-2-benzothiophene-1-carboxylic acid (10) (2.8 g, 8.60 mmol, 67.55% yield) was obtained as a white solid. LCMS: m/z [M+H-Boc]⁺=226.1

### Procedure for preparation of tert-butyl N-methyl-N-(5-methyl-6,7-dihydro-4H-2-benzothiophen-5-yl)carbamate (11)

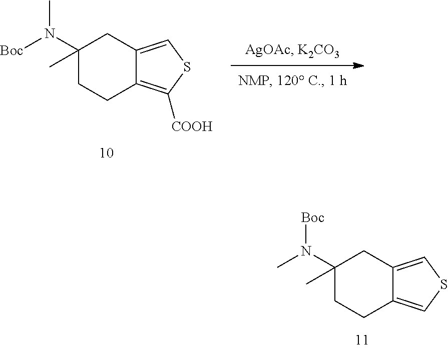 [SMILES: CN(C(=O)OC(C)(C)C)C1(C)CCc2c(csc2C(=O)O)C1 ; CN(C(=O)OC(C)(C)C)C1(C)CCc2cscc2C1]

To a solution of 5-[tert-butoxycarbonyl(methyl)amino]-5-methyl-6,7-dihydro-4H-2-benzothiophene-1-carboxylic acid (10) (2.7 g, 8.30 mmol, 1 eq) in NMP (20 mL) was added AgOAc (138.48 mg, 829.69 μmol, 42.49 μL, 0.1 eq) and K₂CO₃ (3.44 g, 24.89 mmol, 3 eq). The mixture was stirred at 120° C. for 1 hr. The mixture was extracted with EtOAc (100 mL*3). The combined organic phases were washed with brine (100 mL*3), dried over Na₂SO₄, filtered and concentrated at reduced pressure to give a residue. The residue was purified by silica gel chromatography (100-200 mesh silica gel, Petroleum ether/Ethyl acetate=1/0~5/1, Rf=0.50) to afford tert-butyl N-methyl-N-(5-methyl-6,7-dihydro-4H-2-benzothiophen-5-yl) carbamate (11) (1.3 g, 4.62 mmol, 55.68% yield) as a white solid. ¹H NMR (400 MHz, Methanol-d4, 298 K) δ 6.93 (s, 2H), 3.33 (br s, 1H), 3.29 (d, J=2.3 Hz, 1H), 2.94-2.87 (m, 1H), 2.80-2.76 (m, 1H), 2.75 (br s, 1H), 2.73 (s, 3H), 2.68-2.62 (m, 1H), 2.62-2.54 (m, 1H), 1.69 (dt, J=5.5, 12.6 Hz, 1H), 1.45 (s, 9H), 1.41 (s, 3H). LCMS: m/z [M+H-tBu]⁺=226.1

### Procedure for preparation of tert-butyl N-methyl-N-(5-methyl-6,7-dihydro-4H-2-benzothiophen-5-yl) carbamate enantiomer 1 (11a) and enantiomer 2 (11b)

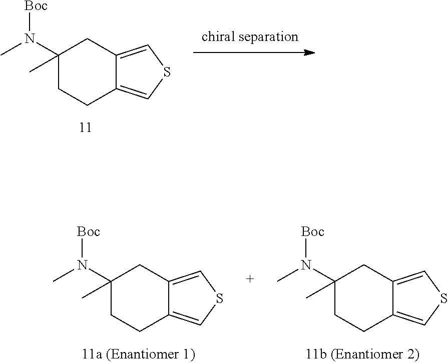 [SMILES: CN(C(=O)OC(C)(C)C)C1(C)CCc2cscc2C1 ; CN(C(=O)OC(C)(C)C)C1(C)CCc2cscc2C1 ; CN(C(=O)OC(C)(C)C)C1(C)CCc2cscc2C1]

Racemic tert-butyl N-methyl-N-(5-methyl-6,7-dihydro-4H-2-benzothiophen-5-yl) carbamate 11 was separated by SFC (column: DAICEL CHIRALPAK IC (250 mm*50 mm, 10 μm); mobile phase: [0.1% NH₃H₂O IPA]; B %: 10%-10%, 3.3 min). Compound tert-butyl N-methyl-N-(5-methyl-6, 7-dihydro-4H-2-benzothiophen-5-yl) carbamate enantiomer 1 (11a) (360 mg, 1.28 mmol, 36.00% yield) was obtained as a white solid. LCMS: m/z [M+H-tBu]⁺=226.2.

Compound tert-butyl N-methyl-N-(5-methyl-6, 7-dihydro-4H-2-benzothiophen-5-yl) carbamate enantiomer 2 (11b) (400 mg, 1.42 mmol, 40.00% yield) was obtained as a white solid. LCMS: m/z [M+H-tBu]⁺=226.2

### Procedure for preparation of N, 5-dimethyl-6,7-dihydro-4H-2-benzothiophen-5-amine hydrochloride enantiomer 1 (101)

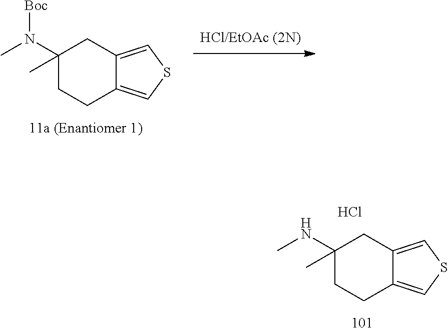 [SMILES: CN(C(=O)OC(C)(C)C)C1(C)CCc2cscc2C1 ; Cl ; CNC1(C)CCc2cscc2C1]

To a solution of tert-butyl N-methyl-N-(5-methyl-6,7-dihydro-4H-2-benzothiophen-5-yl) carbamate enantiomer 1 (11a) (20 mg, 71.07 μmol, 1 eq) in EtOAc (2 mL) was added HCl/EtOAc (2 mL). The mixture was stirred at 25° C. for 1 hr. The reaction mixture was concentrated under reduced pressure. The crude product was triturated with ethyl acetate at 25° C. for 30 min. Compound N, 5-dimethyl-6,7-dihydro-4H-2-benzothiophen-5-amine hydrochloride enantiomer 1 (101) (10 mg, 45.92 μmol, 64.62% yield) was obtained as a white solid. ¹H NMR (400 MHz, METHANOL-d₄) δ 7.11 (br d, J=10.1 Hz, 2H), 3.06-2.94 (m, 2H), 2.94-2.80 (m, 2H), 2.70 (s, 3H), 2.04 (t, J=6.9 Hz, 2H), 1.37 (s, 3H). LCMS: m/z [M+H]⁺=182.2.

### Procedure for preparation of N, 5-dimethyl-6, 7-dihydro-4H-2-benzothiophen-5-amine hydrochloride enantiomer 2 (102)

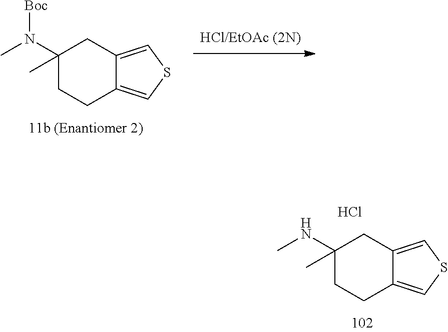 [SMILES: CN(C(=O)OC(C)(C)C)C1(C)CCc2cscc2C1 ; Cl ; CNC1(C)CCc2cscc2C1]

To a solution of tert-butyl N-methyl-N-(5-methyl-6,7-dihydro-4H-2-benzothiophen-5-yl)carbamate enantiomer 2 (11b) (400 mg, 1.42 mmol, 1 eq) in EtOAc (10 mL) was added HCl/EtOAc (4N, 10 mL). The mixture was stirred at 25° C. for 1 hr. The reaction mixture was concentrated under reduced pressure. The residue was purified by SFC (column: DAICEL CHIRALPAK AY (250 mm*50 mm, 10 μm); mobile phase: [CO₂-EtOH (0.1% NH₃H₂O)]; B %: 20%-20%, 3.2 min) and then further purified by prep-HPLC (column: Welch Xtimate C18 150*25 mm*5 μm; mobile phase: [water (HCl)-ACN]; B %: 2%-32%, 8 min) to afford N, 5-dimethyl-6, 7-dihydro-4H-2-benzothiophen-5-amine hydrochloride enantiomer 2 (102) (203.99 mg, 936.77 μmol, 65.90% yield) as a white solid. ¹H NMR (400 MHz, Methanol-d4, 298 K) δ 7.16-7.07 (m, 2H), 3.06-2.94 (m, 2H), 2.94-2.81 (m, 2H), 2.70 (s, 3H), 2.04 (dd, J=6.6, 7.3 Hz, 2H), 1.36 (s, 3H). LCMS: m/z [M+H]⁺=182.2.

### Synthetic scheme for preparation of racemic N, 5-dimethyl-6,7-dihydro-4H-2-benzothiophen-5-amine hydrochloride (103)

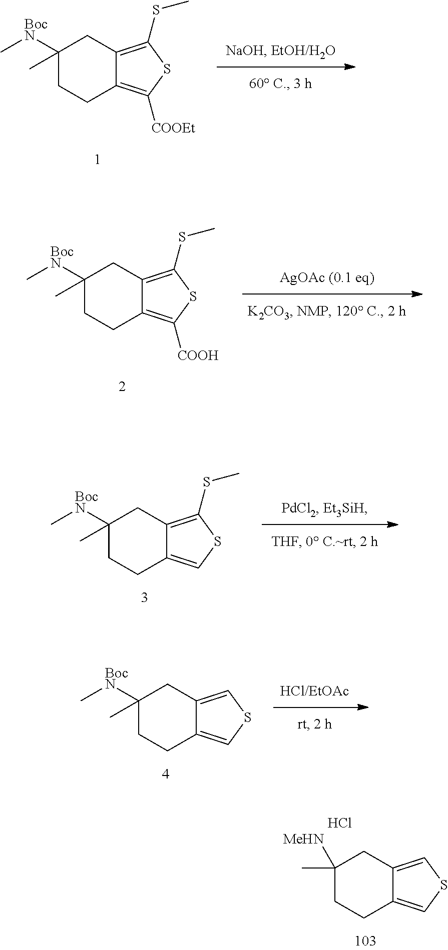

### Procedure for the synthesis of 5-[tert-butoxycarbonyl(methyl)amino]-5-methyl-3-methylsulfanyl-6,7-dihydro-4H-2-benzothiophene-1-carboxylic acid (2)

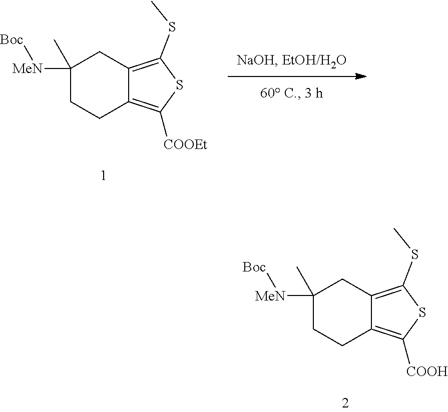 [SMILES: CCOC(=O)c1sc(SC)c2c1CCC(C)(N(C)C(=O)OC(C)(C)C)C2 ; CSc1sc(C(=O)O)c2c1CC(C)(N(C)C(=O)OC(C)(C)C)CC2]

To a solution of ethyl 5-[tert-butoxycarbonyl(methyl)amino]-5-methyl-3-methylsulfanyl-6,7-dihydro-4H-2-benzothiophene-1-carboxylate (1) (1.5 g, 3.75 mmol, 1 eq) in EtOH (8 mL) was added NaOH (300.30 mg, 7.51 mmol, 2 eq) and H₂O (8 mL). The mixture was stirred at 60° C. for 3 hr. The reaction mixture was concentrated under reduced pressure to remove EtOH. The residue was diluted with aq. NaHCO₃ saturated solution (10 mL), aq. citric acid was added into the mixture to adjust pH to 5, then extracted with EtOAc (10 mL*3). The combined organic layers were dried over Na₂SO₄, filtered and concentrated under reduced pressure to give the crude product 5-[tert-butoxycarbonyl(methyl)amino]-5-methyl-3-methylsulfanyl-6,7-dihydro-4H-2-benzothiophene-1-carboxylic acid (2) (1.28 g, crude). LCMS: m/z [M+H-Boc]⁺=272.1.

### Procedure for the synthesis of tert-butyl N-methyl-N-(5-methyl-3-methylsulfanyl-6,7-dihydro-4H-2-benzothiophen-5-yl)carbamate (3)

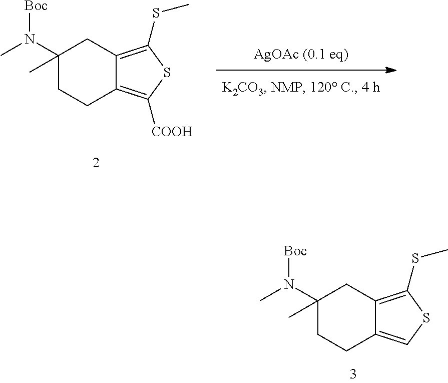

To a mixture of 5-[tert-butoxycarbonyl(methyl)amino]-5-methyl-3-methylsulfanyl-6,7-dihydro-4H-2-benzothiophene-1-carboxylic acid (2) (380 mg, 1.02 mmol, 1 eq) and K₂CO₃ (424.09 mg, 3.07 mmol, 3 eq) in NMP (4 mL) was added AgOAc (17.07 mg, 102.28 mmol, 5.24 mL, 0.1 eq) under N₂. The mixture was stirred at 120° C. for 4 hours. The mixture was extracted with EtOAc (50 mL*3). The combined organic phase was washed with brine (10 mL*3), dried over Na₂SO₄, filtered and concentrated at reduced pressure to give a residue. Compound tert-butyl N-methyl-N-(5-methyl-3-methylsulfanyl-6,7-dihydro-4H-2-benzothiophen-5-yl)carbamate (3) (300 mg, crude) was obtained as a yellow gum.

### Procedure for preparation of tert-butyl N-methyl-N-(5-methyl-3-methylsulfanyl-6,7-dihydro-4H-2-benzothiophen-5-yl)carbamate (3)

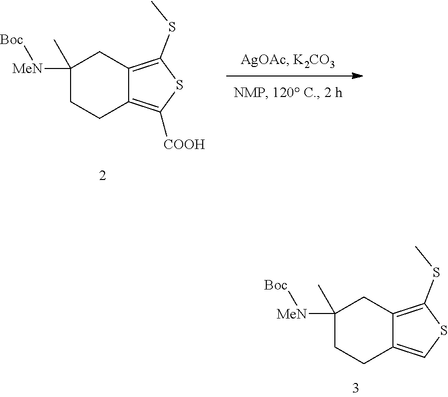 [SMILES: CSc1sc(C(=O)O)c2c1CC(C)(N(C)C(=O)OC(C)(C)C)CC2 ; CSc1scc2c1CC(C)(N(C)C(=O)OC(C)(C)C)CC2]

To a solution of 5-[tert-butoxycarbonyl(methyl)amino]-5-methyl-3-methylsulfanyl-6,7-dihydro-4H-2-benzothiophene-1-carboxylic acid (2) (440 mg, 1.18 mmol, 1 eq) in NMP (5 mL) was added AgOAc (19.77 mg, 118.43 μmol, 6.07 μL, 0.1 eq) and K₂CO₃ (491.05 mg, 3.55 mmol, 3 eq). The mixture was stirred at 120° C. for 2 hr. The reaction mixture was poured into H₂O (20 mL) and extracted with EtOAc (20 mL*3). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~50% Ethyl acetate/Petroleum ether gradient @40 mL/min) to give tert-butyl N-methyl-N-(5-methyl-3-methylsulfanyl-6,7-dihydro-4H-2-benzothiophen-5-yl)carbamate (3) (140 mg, 427.48 μmol, 36.09% yield) as yellow oil. ¹H NMR (400 MHz, CHLOROFORM-d) δ 6.90 (s, 1H), 3.36 (dd, J=2.4, 17.1 Hz, 1H), 3.02-2.87 (m, 1H), 2.77-2.56 (m, 5H), 2.37 (s, 3H), 1.69-1.57 (m, 2H), 1.47 (s, 9H), 1.44 (s, 3H).

### Procedure for the synthesis of tert-butyl N-methyl-N-(5-methyl-6,7-dihydro-4H-2-benzothiophen-5-yl)carbamate (4)

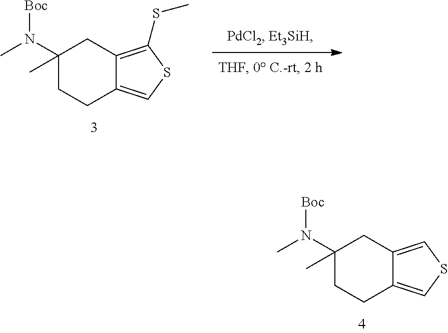

To a solution of tert-butyl N-methyl-N-(5-methyl-3-methylsulfanyl-6,7-dihydro-4H-2-benzothiophen-5-yl)carbamate (3) (300 mg, 916.02 μmol, 1 eq) in THF (5 mL) was added PdCl₂ (16.24 mg, 91.60 mmol, 0.1 eq) and Et₃SiH (213.02 mg, 1.83 mmol, 292.62 mL, 2 eq) at 0° C. The mixture was stirred at 20° C. for 2 hr. The reaction mixture was filtered and concentrated under reduced pressure to give a residue, which was purified by prep-HPLC (column: YMC Triart C18 150*25 mm*5 μm; mobile phase: [water(HCl)-ACN]; B %: 68%-98%, 10 min) to give tert-butyl N-methyl-N-(5-methyl-6,7-dihydro-4H-2-benzothiophen-5-yl)carbamate (4) (126 mg, 447.74 mmol, 48.88% yield) as a white solid. LCMS: m/z [M+H-tBu]⁺=226.2.

### Procedure for the synthesis of racemic N, 5-dimethyl-6,7-dihydro-4H-2-benzothiophen-5-amine hydrochloride (103)

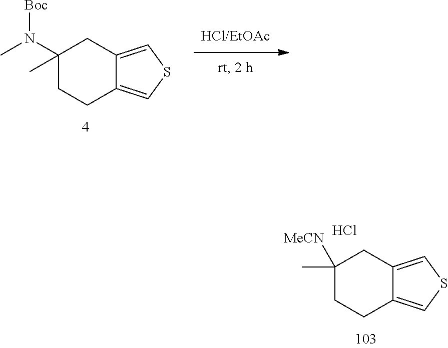

To a solution of tert-butyl N-methyl-N-(5-methyl-6,7-dihydro-4H-2-benzothiophen-5-yl)carbamate (4) (126 mg, 447.74 mmol, 1 eq) in EtOAc (0.5 mL) was added HCl/EtOAc (1 mL). The mixture was stirred at 20° C. for 2 hr. The reaction mixture was filtered to give racemic N, 5-dimethyl-6,7-dihydro-4H-2-benzothiophen-5-amine hydrochloride (103) (94.63 mg, 434.56 mmol, 97.06% yield) as an off-white solid. LCMS: m/z [M+H]⁺=182.1. ¹H NMR (400 MHz, METHANOL-d₄) δ 7.15 (s, 1H), 7.10 (s, 1H), 3.06-2.81 (m, 4H), 2.70 (s, 3H), 2.08-1.98 (m, 2H), 1.36 (s, 3H)

### Synthetic scheme for preparation of N, 1-dimethyl-3-(trifluoromethyl)-4,5,6,7-tetrahydro-2-benzothiophen-5-amine hydrochloride enantiomer 1 (104), and enantiomer 2 (105) and N, 3-dimethyl-1-(trifluoromethyl)-4,5,6,7-tetrahydro-2-benzothiophen-5-amine hydrochloride enantiomer 1 (106) and enantiomer 2 (107)

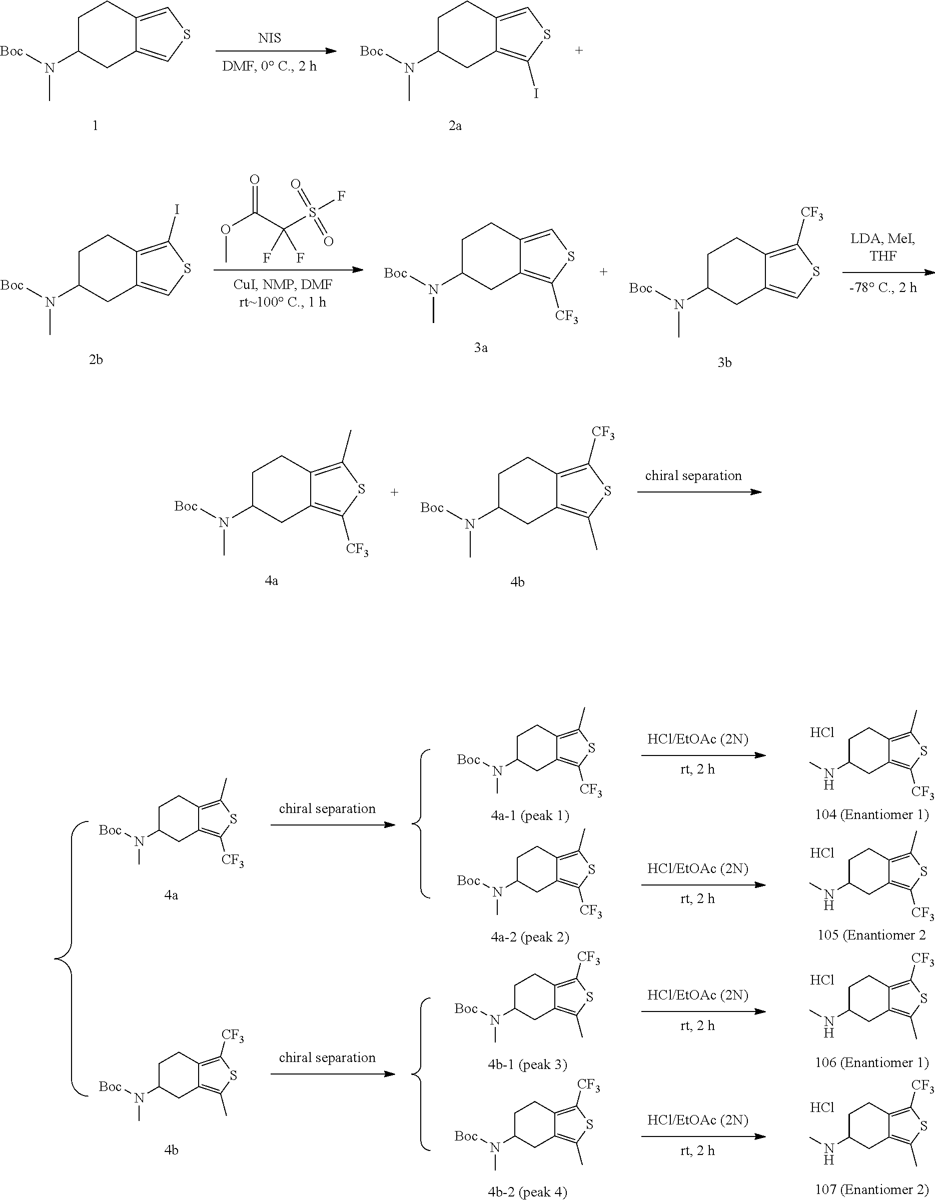

### Procedure for preparation of tert-butyl N-(3-iodo-4,5,6,7-tetrahydro-2-benzothiophen-5-yl)-N-methyl-carbamate (2a) and tert-butyl N-(-iodo-4,5,6,7-tetrahydro-2-benzothiophen-5-yl)-N-methyl-carbamate (2b)

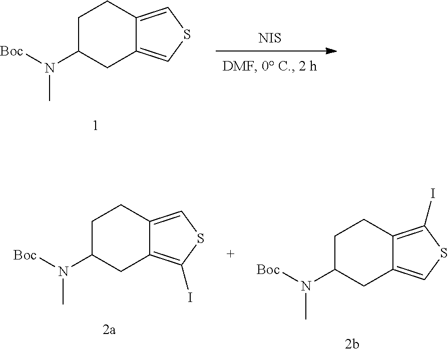 [SMILES: CN(C(=O)OC(C)(C)C)C1CCc2cscc2C1 ; "NIS" ; CN(C(=O)OC(C)(C)C)C1CCc2csc(I)c2C1 ; CN(C(=O)OC(C)(C)C)C1CCc2c(csc2I)C1]

To a solution of tert-butyl N-methyl-N-(4,5,6,7-tetrahydro-2-benzothiophen-5-yl) carbamate (1) (2 g, 7.48 mmol, 1 eq) in DMF (30 mL) was added NIS (2.02 g, 8.98 mmol, 1.2 eq). The mixture was stirred at 0° C. for 2 hr. The reaction mixture was quenched by addition H₂O (30 mL), and then extracted with EtOAc (30 mL*3). The combined organic layers were washed with NaCl saturated solution (30 mL*2), dried over Na₂SO₄, filtered and concentrated under reduced pressure. The residue was purified by flash silica gel chromatography (ISCO®; 20 SepaFlash® Silica Flash Column, eluent 0~10% Ethyl acetate/Petroleum) to give tert-butyl N-(3-iodo-4,5,6,7-tetrahydro-2-benzothiophen-5-yl)-N-methyl-carbamate (2a) and tert-butyl N-(1-iodo-4,5,6,7-tetrahydro-2-benzothiophen-5-yl)-N-methyl-carbamate (2b) (2.4 g, 6.10 mmol, 81.59% yield) as yellow oil.

### Procedure for preparation of tert-butyl N-methyl-N-[3-(trifluoromethyl)-4,5,6,7-tetrahydro-2-benzothiophen-5-yl]carbamate (3a) and tert-butyl N-methyl-N-[1-(trifluoromethyl)-4,5,6,7-tetrahydro-2-benzothiophen-5-yl]carbamate (3b)

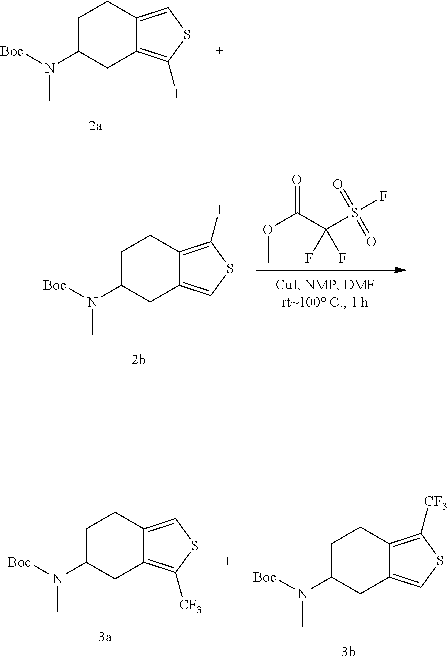

To a solution of tert-butyl N-(3-iodo-4,5,6,7-tetrahydro-2-benzothiophen-5-yl)-N-methyl-carbamate (2a) and tert-butyl N-(1-iodo-4,5,6,7-tetrahydro-2-benzothiophen-5-yl)-N-methyl-carbamate (2b) (2.30 g, 5.85 mmol, 1 eq) in DMF (20 mL) was added CuI (1.23 g, 6.43 mmol, 1.1 eq) and NMP (2.90 g, 29.24 mmol, 2.84 mL, 5 eq) at 25° C. After addition, the mixture was stirred at this temperature for 10 min, and then methyl 2,2-difluoro-2-fluorosulfonyl-acetate (5.62 g, 29.24 mmol, 3.72 mL, 5 eq) was added dropwise at 25° C. The resulting mixture was stirred at 100° C. for 1 hr. The reaction mixture was poured into H₂O (50 mL) and extracted with EtOAc (20 mL*2). The combined organic phase was washed with brine (10 mL), dried over anhydrous sodium sulfate, filtered and concentrated under vacuum to give a residue. The residue was purified by flash silica gel chromatography (ISCO®; 40 g SepaFlash® Silica Flash Column, eluent 0~10% Ethyl acetate/Petroleum) and then by prep-HPLC (neutral condition) to give tert-butyl N-methyl-N-[3-(trifluoromethyl)-4,5,6,7-tetrahydro-2-benzothiophen-5-yl]carbamate (3a) and tert-butyl N-methyl-N-[1-(trifluoromethyl)-4,5,6,7-tetrahydro-2-benzothiophen-5-yl]carbamate (3b) (1.3 g, 3.88 mmol, 66.28% yield) as yellow oil.

### Procedure for preparation of tert-butyl N-methyl-N-[1-methyl-3-(trifluoromethyl)-4,5,6,7-tetrahydro-2-benzothiophen-5-yl]carbamate (4a) and tert-butyl N-methyl-N-[3-methyl-1-(trifluoromethyl)-4,5,6,7-tetrahydro-2-benzothiophen-5-yl]carbamate (4b)

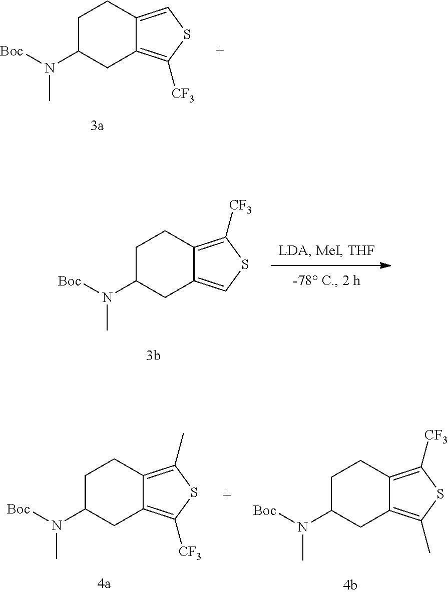 [SMILES: CN(C(=O)OC(C)(C)C)C1CCc2csc(C(F)(F)F)c2C1 ; CN(C(=O)OC(C)(C)C)C1CCc2c(csc2C(F)(F)F)C1 ; Cc1sc(C(F)(F)F)c2c1CCC(N(C)C(=O)OC(C)(C)C)C2 ; Cc1sc(C(F)(F)F)c2c1CC(N(C)C(=O)OC(C)(C)C)CC2]

To a solution of tert-butyl N-methyl-N-[3-(trifluoromethyl)-4,5,6,7-tetrahydro-2-benzothiophen-5-yl]carbamate (3a) and tert-butyl N-methyl-N-[1-(trifluoromethyl)-4,5,6,7-tetrahydro-2-benzothiophen-5-yl]carbamate (3b) (1.3 g, 3.88 mmol, 1 eq) in THF (20 mL) was added dropwise LDA (2 M, 2.91 mL, 1.5 eq) at −78° C. After addition, the mixture was stirred at this temperature for 0.5 hr, and then CH₃I (825.26 mg, 5.81 mmol, 361.96 μL, 1.5 eq) was added dropwise at −78° C. The resulting mixture was stirred at −78° C. for 2 hr. The reaction mixture was quenched by addition NH₄Cl saturated solution (50 ml) at 0° C., and then diluted with H₂O (20 mL) and extracted with EtOAc (50 mL*3). The combined organic layers were dried over Na₂SO₄, filtered and concentrated under reduced pressure to give a residue. The residue was purified by prep-HPLC (column: Kromasil Eternity XT 250*80 mm*10 μm; mobile phase: [water (NH₃H₂O+NH₄HCO₃)-MeOH]; B %: 60%-90%, 20 min) to give tert-butyl N-methyl-N-[1-methyl-3-(trifluoromethyl)-4,5,6,7-tetrahydro-2-benzothiophen-5-yl]carbamate (4a) and tert-butyl N-methyl-N-[3-methyl-1-(trifluoromethyl)-4,5,6,7-tetrahydro-2-benzothiophen-5-yl]carbamate (4b) (1.1 g, 3.15 mmol, 81.22% yield) as yellow oil. The mixture was separated by SFC (column: REGIS (s,s) WHELK-01 (250 mm*50 mm, 10 μm); mobile phase: [Hexane-IPA (0.1% NH₃)]; B %: 10%-10%, 4.5 min). Compound tert-butyl N-methyl-N-[1-methyl-3-(trifluoromethyl)-4,5,6,7-tetrahydro-2-benzothiophen-5-yl]carbamate (4a) (320 mg, 915.83 μmol, 29.09% yield) was obtained as a white solid. Compound tert-butyl N-methyl-N-[3-methyl-1-(trifluoromethyl)-4,5,6,7-tetrahydro-2-benzothiophen-5-yl]carbamate (4b) (290 mg, 829.97 μmol, 26.36% yield) was obtained as yellow oil.

### Procedure for preparation of tert-butyl N-methyl-N-[1-methyl-3-(trifluoromethyl)-4,5,6,7-tetrahydro-2-benzothiophen-5-yl]carbamate enantiomer 1 (peak 1, 4a-1) and enantiomer 2 (peak2, 4a-2) and tert-butyl N-methyl-N-[3-methyl-1-(trifluoromethyl)-4,5,6,7-tetrahydro-2-benzothiophen-5-yl]carbamate enantiomer 1 (peak 3, 4b-1) and enantiomer 2 (peak 4, 4b-2)

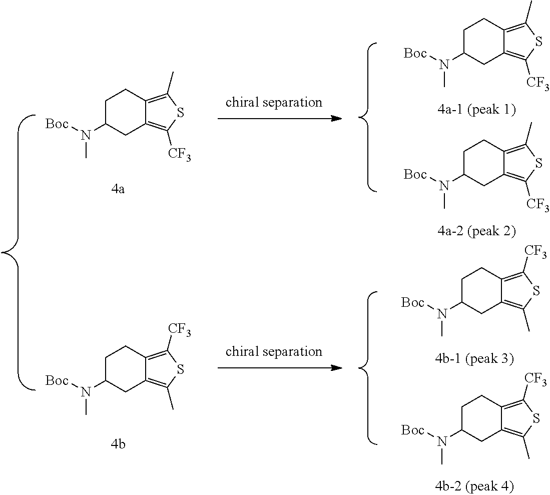 [SMILES: Cc1sc(C(F)(F)F)c2c1CCC(N(C)C(=O)OC(C)(C)C)C2 ; Cc1sc(C(F)(F)F)c2c1CCC(N(C)C(=O)OC(C)(C)C)C2 ; Cc1sc(C(F)(F)F)c2c1CCC(N(C)C(=O)OC(C)(C)C)C2 ; Cc1sc(C(F)(F)F)c2c1CC(N(C)C(=O)OC(C)(C)C)CC2 ; Cc1sc(C(F)(F)F)c2c1CC(N(C)C(=O)OC(C)(C)C)CC2 ; Cc1sc(C(F)(F)F)c2c1CC(N(C)C(=O)OC(C)(C)C)CC2]

Racemic 4a was separated by SFC (column: DAICEL CHIRALPAK IG (250 mm*50 mm, 10 μm); mobile phase: [CO₂-MeOH (0.1% NH₃H₂O)]; B %: 10%-10%, 4.1 min). Racemic 4b was separated by SFC (column: DAICEL CHIRALPAK IG (250 mm*50 mm, 10 μm); mobile phase: [CO₂-MeOH (0.1% NH₃H₂O)]; B %: 10%-10%, 3.5 min). Compound tert-butyl N-methyl-N-[1-methyl-3-(trifluoromethyl)-4,5,6,7-tetrahydro-2-benzothiophen-5-yl]carbamate enantiomer 1 (peak 1, 4a-1) (100 mg, 286.20 μmol, 31.25% yield) was obtained as a white solid. Compound tert-butyl N-methyl-N-[1-methyl-3-(trifluoromethyl)-4,5,6,7-tetrahydro-2-benzothiophen-5-yl]carbamate enantiomer 2 (peak 2, 4a-2) (120 mg, 343.44 μmol, 37.50% yield) was obtained as a white solid. Compound tert-butyl N-methyl-N-[3-methyl-1-(trifluoromethyl)-4,5,6,7-tetrahydro-2-benzothiophen-5-yl]carbamate enantiomer 1 (peak 3, 4b-1) (100 mg, 286.20 μmol, 31.25% yield) was obtained as a white solid. Compound tert-butyl N-methyl-N-[3-methyl-1-(trifluoromethyl)-4,5,6,7-tetrahydro-2-benzothiophen-5-yl]carbamate enantiomer 2 (peak 4, 4b-2) (100 mg, 286.20 μmol, 31.25% yield) was obtained as a white solid.

### Procedure for preparation of N, 1-dimethyl-3-(trifluoromethyl)-4,5,6,7-tetrahydro-2-benzothiophen-5-amine hydrochloride enantiomer 1 (104)

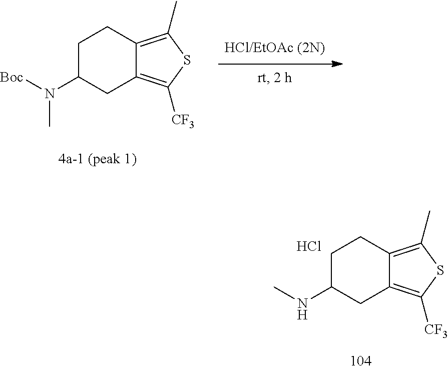 [SMILES: Cc1sc(C(F)(F)F)c2c1CCC(N(C)C(=O)OC(C)(C)C)C2 ; CNC1CCc2c(C)sc(C(F)(F)F)c2C1 ; Cl]

To a solution of tert-butyl N-methyl-N-[1-methyl-3-(trifluoromethyl)-4,5,6,7-tetrahydro-2-benzothiophen-5-yl]carbamate peak 1 (4a-1) (100 mg, 286.20 μmol, 1 eq) in EtOAc (1 mL) was added HCl/EtOAc (4 M, 1 mL, 13.98 eq). The mixture was stirred at 25° C. for 2 hr. The mixture was filtrered, the filter cake was dried. The product was triturated with EtOAc at 25° C. for 10 min filtrered, the filter cake was dried to afford N, 1-dimethyl-3-(trifluoromethyl)-4,5,6,7-tetrahydro-2-benzothiophen-5-amine enantiomer 1 (104) (74.21 mg, 254.50 μmol, 88.93% yield) as a white solid. ¹H NMR (400 MHz, METHANOL-d₄) δ 3.56-3.44 (m, 1H), 3.43-3.33 (m, 1H), 2.92-2.75 (m, 5H), 2.71-2.60 (m, 1H), 2.38 (s, 3H), 2.35-2.26 (m, 1H), 1.93-1.79 (m, 1H), LC-MS: m/z [M+H]⁺=250.1

### Procedure for preparation of N, 1-dimethyl-3-(trifluoromethyl)-4,5,6,7-tetrahydro-2-benzothiophen-5-amine hydrochloride enantiomer 2 (105)

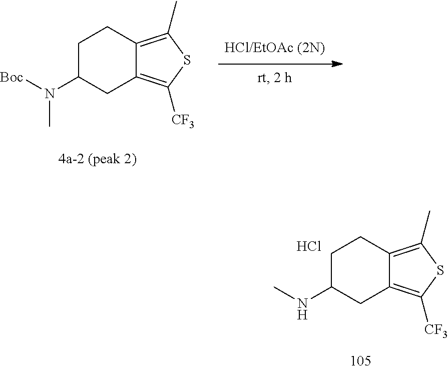 [SMILES: Cc1sc(C(F)(F)F)c2c1CCC(N(C)C(=O)OC(C)(C)C)C2 ; CNC1CCc2c(C)sc(C(F)(F)F)c2C1 ; Cl]

To a solution of tert-butyl N-methyl-N-[1-methyl-3-(trifluoromethyl)-4,5,6,7-tetrahydro-2-benzothiophen-5-yl]carbamate peak 2 (4a-2) (100 mg, 286.20 μmol, 1 eq) in EtOAc (1 mL) was added HCl/EtOAc (4 M, 1 mL, 13.98 eq). The mixture was stirred at 25° C. for 2 hr. The mixture was filtered, the filter cake was dried. The product was triturated with EtOAc at 25° C. for 10 min. The mixture was filtered, the filter cake was dried to afford N, 1-dimethyl-3-(trifluoromethyl)-4,5,6,7-tetrahydro-2-benzothiophen-5-amine hydrochloride enantiomer 2 (105) (69.98 mg, 235.10 μmol, 82.15% yield) as a white solid. ¹H NMR (400 MHz, METHANOL-d₄) δ 3.54-3.44 (m, 1H), 3.42-3.34 (m, 1H), 2.95-2.74 (m, 5H), 2.60-2.75 (m, 1H), 2.38 (s, 3H), 2.35-2.26 (m, 1H), 1.94-1.80 (m, 1H). LC-MS: m/z [M+H]⁺=250.2

### Procedure for preparation of N, 3-dimethyl-1-(trifluoromethyl)-4,5,6,7-tetrahydro-2-benzothiophen-5-amine hydrochloride enantiomer 1 (106)

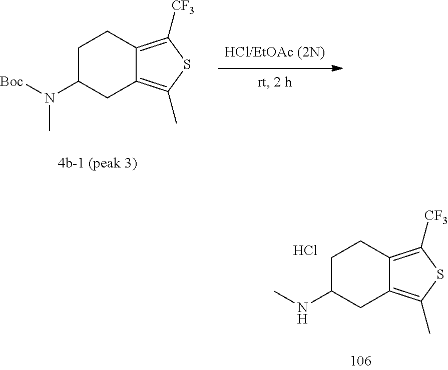 [SMILES: Cc1sc(C(F)(F)F)c2c1CC(N(C)C(=O)OC(C)(C)C)CC2 ; CNC1CCc2c(C(F)(F)F)sc(C)c2C1 ; Cl]

To a solution of tert-butyl N-methyl-N-[3-methyl-1-(trifluoromethyl)-4,5,6,7-tetrahydro-2-benzothiophen-5-yl]carbamate peak 3 (4b-1) (90.00 mg, 257.58 μmol, 1 eq) in EtOAc (1 mL) was added HCl/EtOAc (4 M, 1 mL, 15.53 eq). The mixture was stirred at 25° C. for 2 hr. The mixture was then filtered, the filter cake was dried. The product was triturated with EtOAc at 25° C. for 10 min. The mixture was filtered, the filter cake was dried to afford N, 3-dimethyl-1-(trifluoromethyl)-4,5,6,7-tetrahydro-2-benzothiophen-5-amine hydrochloride enantiomer 1 (106) (69.39 mg, 233.12 μmol, 90.50% yield) as a white solid. ¹H NMR (400 MHz, METHANOL-d₄) δ 3.54-3.41 (m, 1H), 3.26-3.03 (m, 2H), 2.90-2.76 (m, 4H), 2.64-2.53 (m, 1H), 2.40 (s, 3H), 2.33-2.22 (m, 1H), 1.91-1.77 (m, 1H). LC-MS: m/z [M+H]⁺=250.1

### Procedure for preparation of N, 3-dimethyl-1-(trifluoromethyl)-4,5,6,7-tetrahydro-2-benzothiophen-5-amine hydrochloride enantiomer 2 (107)

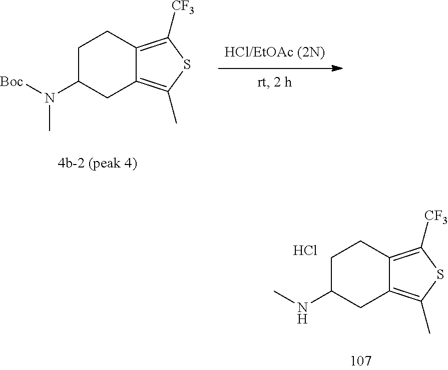 [SMILES: Cc1sc(C(F)(F)F)c2c1CC(N(C)C(=O)OC(C)(C)C)CC2 ; CNC1CCc2c(C(F)(F)F)sc(C)c2C1 ; Cl]

To a solution of tert-butyl N-methyl-N-[3-methyl-1-(trifluoromethyl)-4,5,6,7-tetrahydro-2-benzothiophen-5-yl]carbamate peak 4 (4b-2) (120 mg, 343.44 μmol, 1 eq) in EtOAc (1 mL) was added HCl/EtOAc (4 M, 1.20 mL, 13.98 eq). The mixture was stirred at 25° C. for 2 hr. The mixture was filtered, the filter cake was dried. The product was triturated with EtOAc at 25° C. for 10 min. The mixture was filtered, the filter cake was dried to afford N, 3-dimethyl-1-(trifluoromethyl)-4,5,6,7-tetrahydro-2-benzothiophen-5-amine hydrochloride enantiomer 2 (107) (99.53 mg, 341.34 μmol, 99.39% yield) as a white solid. ¹H NMR (400 MHz, METHANOL-d₄) δ 3.54-3.42 (m, 1H), 3.24-3.03 (m, 2H), 2.92-2.75 (m, 4H), 2.64-2.53 (m, 1H), 2.40 (s, 3H), 2.33-2.23 (m, 1H), 1.92-1.77 (m, 1H). LC-MS: m/z [M+H]⁺=250.1

### Synthetic scheme for preparation of 3-bromo-N-methyl-4,5,6,7-tetrahydrobenzothiophen-5-amine hydrochloride (108)

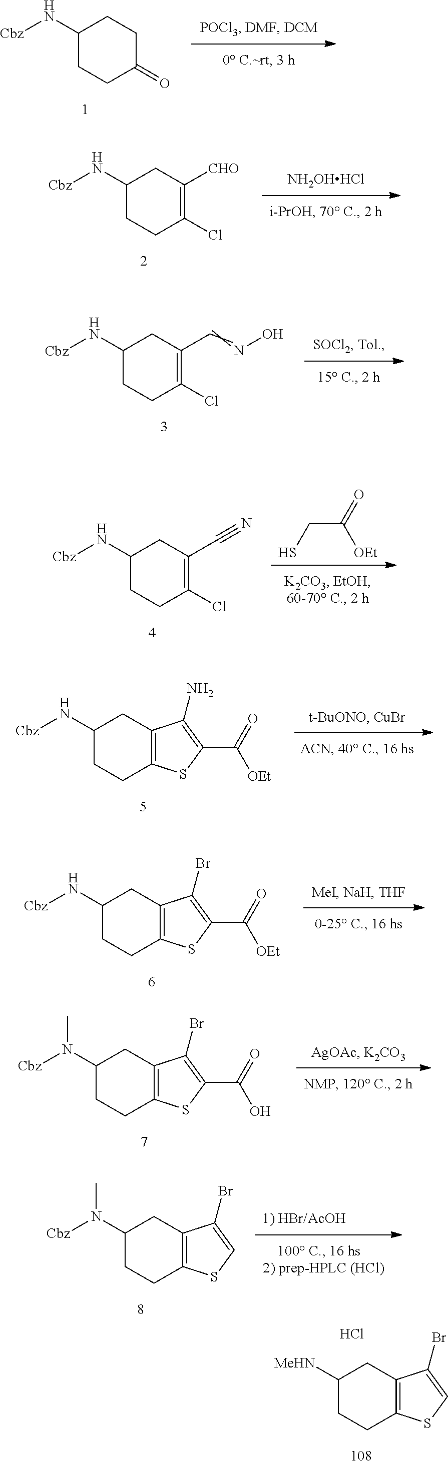

### Procedure for preparation of benzyl N-(4-chloro-3-formyl-cyclohex-3-en-1-yl)carbamate (2)

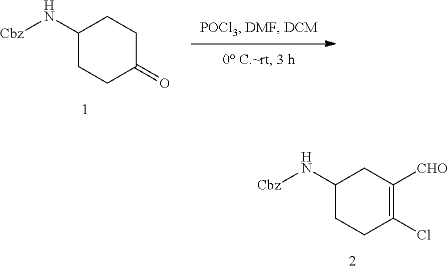 [SMILES: O=C1CCC(NC(=O)OCc2ccccc2)CC1 ; O=CC1=C(Cl)CCC(NC(=O)OCc2ccccc2)C1]

To a solution of DMF (26.60 g, 363.95 mmol, 28.00 mL, 4.5 eq) in DCM (100 mL) was added POCl₃ (49.60 g, 323.51 mmol, 30.15 mL, 4 eq) in DCM (50 mL) slowly at 0° C., the mixture was stirred at 0° C. for 1 hour, then benzyl N-(4-oxocyclohexyl)carbamate (1) (20 g, 80.88 mmol, 1 eq) in DCM (50 mL) was added, the mixture was stirred at 0~20° C. for 2 hours. The reaction solution was slowly added to 20% aq. KH₂PO₄ (1 L), then solid NaHCO₃ was added to adjust the pH to 7-8, then the mixture was extracted with DCM (200 mL*2), the organic layer was separated and washed with brine (200 mL), dried over anhydrous Na₂SO₄, filtered and concentrated to get a residue. The residue was purified by silica gel column (Petroleum ether/Ethyl acetate=3:1) to afford benzyl N-(4-chloro-3-formyl-cyclohex-3-en-1-yl)carbamate (2) (17 g, 57.87 mmol, 71.56% yield) as yellow oil.

### Procedure for preparation of benzyl N-[4-chloro-3-[hydroxyiminomethyl]cyclohex-3-en-1-yl]carbamate (3)

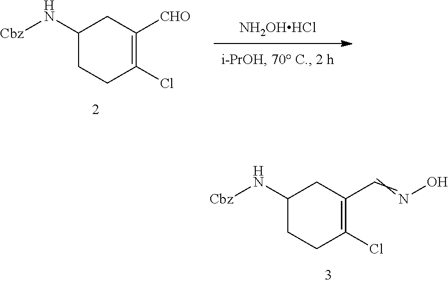 [SMILES: O=CC1=C(Cl)CCC(NC(=O)OCc2ccccc2)C1 ; "NH2OH·HCl" ; O=C(NC1CCC(Cl)=C(C=NO)C1)OCc1ccccc1]

To a solution of benzyl N-(4-chloro-3-formyl-cyclohex-3-en-1-yl)carbamate (2) (17 g, 57.87 mmol, 1 eq) in i-PrOH (120 mL) was added NH₂OH·HCl (4.02 g, 57.87 mmol, 1 eq), the mixture was stirred at 70° C. for 2 hours. The reaction was concentrated to afford benzyl N-[4-chloro-3-[hydroxyiminomethyl]cyclohex-3-en-1-yl]carbamate (3) (17 g, crude) as yellow solid and used directly into next step without further purification. LCMS: m/z [M+H]⁺=309.2

### Procedure for preparation of benzyl N-(4-chloro-3-cyano-cyclohex-3-en-1-yl)carbamate(4)

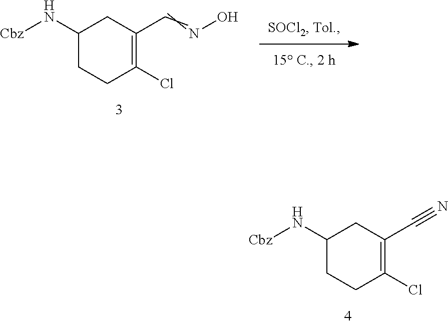 [SMILES: O=C(NC1CCC(Cl)=C(C=NO)C1)OCc1ccccc1 ; "SOCl2, Tol.," ; N#CC1=C(Cl)CCC(NC(=O)OCc2ccccc2)C1]

To a solution of benzyl N-[4-chloro-3-[hydroxyiminomethyl]cyclohex-3-en-1-yl]carbamate (3) (17 g, 55.06 mmol, 1 eq) in Tol. (100 mL) was added SOCl2 (19.65 g, 165.18 mmol, 12.00 mL, 3 eq), the mixture was stirred at 15° C. for 2 hours. The reaction was concentrated to afford benzyl N-(4-chloro-3-cyano-cyclohex-3-en-1-yl)carbamate (4) (16 g, crude) as yellow oil. LCMS: m/z [M+H]⁺=291.2.

### Procedure for preparation of ethyl 3-amino-5-(benzyloxycarbonylamino)-4,5,6,7-tetrahydrobenzothiophene-2-carboxylate(5)

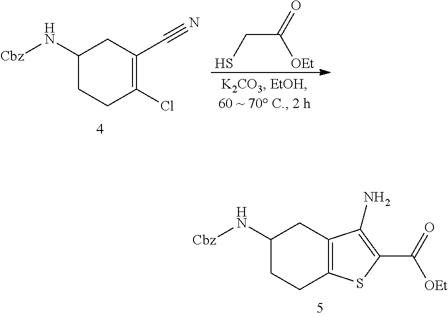 [SMILES: N#CC1=C(Cl)CCC(NC(=O)OCc2ccccc2)C1 ; CCOC(=O)CS ; CCOC(=O)c1sc2c(c1N)CC(NC(=O)OCc1ccccc1)CC2]

To a solution of benzyl N-(4-chloro-3-cyano-cyclohex-3-en-1-yl)carbamate (4) (16 g, 55.03 mmol, 1 eq) in EtOH (150 mL) was added K₂CO₃ (22.82 g, 165.09 mmol, 3 eq) and ethyl 2-sulfanylacetate (7.14 g, 59.43 mmol, 6.52 mL, 1.08 eq), the mixture was stirred at 60-70° C. for 2 hours. The reaction was cooled to rt, poured into water (400 mL), extracted with EtOAc (300 mL*2), the organic layers were combined and washed with brine (200 mL*2), dried over anhydrous Na₂SO₄, filtered and concentrated to get a residue. The residue was purified by silica gel column (Petroleum ether/Ethyl acetate=3:1 to 1:1) to afford ethyl 3-amino-5-(benzyloxycarbonylamino)-4,5,6,7-tetrahydrobenzothiophene-2-carboxylate (5) (16 g, 42.73 mmol, 77.65% yield) as yellow oil. LCMS: m/z [M+H]⁺=375.1.

### Procedure for preparation of ethyl 5-(benzyloxycarbonylamino)-3-bromo-4,5,6,7-tetrahydrobenzothiophene-2-carboxylate (6)

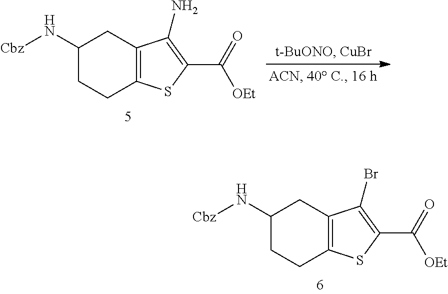 [SMILES: CCOC(=O)c1sc2c(c1N)CC(NC(=O)OCc1ccccc1)CC2 ; CCOC(=O)c1sc2c(c1Br)CC(NC(=O)OCc1ccccc1)CC2]

To a solution of ethyl 3-amino-5-(benzyloxycarbonylamino)-4,5,6,7-tetrahydrobenzothiophene-2-carboxylate (5) (1.5 g, 4.01 mmol, 1 eq) in can (15 mL) was added CuBr (1.02 g, 7.13 mmol, 1.78 eq) at 20° C., then t-BuONO (433.74 mg, 4.21 mmol, 500.27 μL, 1.05 eq) was added with stirring, and the solution was stirred at 40° C. for 16 hours. The reaction was cooled to rt., quenched with water (50 mL), diluted with Ethyl acetate (50 mL), filtered and the filtrate was extracted with Ethyl acetate (50 mL), the organic layers were combined and washed with brine (80 mL), dried over anhydrous Na₂SO₄, filtered and concentrated to get a residue. The residue was purified by prep-HPLC (column: Phenomenex luna C18 150*40 mm*15 μm; mobile phase: [water (TFA ACN]; gradient: 55%-85% B over 15 min). Compound ethyl 5-(benzyloxycarbonylamino)-3-bromo-4,5,6,7-tetrahydrobenzothiophene-2-carboxylate (6) (0.61 g, 1.39 mmol, 34.74% yield) was obtained as light red oil. LCMS: m/z [M+Na]⁺=460.1.

### Procedure for preparation of 5-[benzyloxycarbonyl(methyl)amino]-3-bromo-4,5,6,7-tetrahydrobenzothiophene-2-carboxylic acid (7)

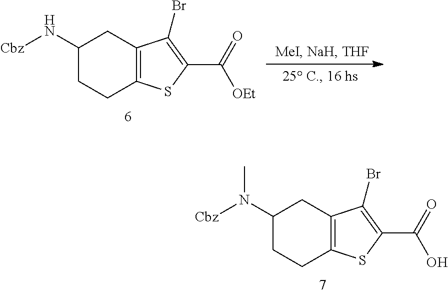 [SMILES: CCOC(=O)c1sc2c(c1Br)CC(NC(=O)OCc1ccccc1)CC2 ; CN(C(=O)OCc1ccccc1)C1CCc2sc(C(=O)O)c(Br)c2C1]

To a solution of ethyl 5-(benzyloxycarbonylamino)-3-bromo-4,5,6,7-tetrahydrobenzothiophene-2-carboxylate (6) (0.52 g, 1.19 mmol, 1 eq) in THF (6 mL) was added NaH (94.90 mg, 2.37 mmol, 60% suspension in mineral oil, 2 eq) at 0° C. under N₂, then MeI (336.77 mg, 2.37 mmol, 147.71 μL, 2 eq) was added, the mixture was stirred at 25° C. for 16 hours. The reaction mixture was quenched by addition aq. sat. NH₄Cl (50 mL) at 0° C., and then diluted with H₂O (40 mL) and extracted with EtOAc (40 mL*2). The combined organic layers were washed with aq. sat.NaCl (80 mL), dried over anhydrous Na₂SO₄, filtered and concentrated under reduced pressure to get compound 5-[benzyloxycarbonyl(methyl)amino]-3-bromo-4,5,6,7-tetrahydrobenzothiophene-2-carboxylic acid (7) (0.6 g, crude) as a red solid. LCMS: m/z [M+Na]⁺=446.1.

### Procedure for preparation of benzyl N-(3-bromo-4,5,6,7-tetrahydrobenzothiophen-5-yl)-N-methyl-carbamate (8)

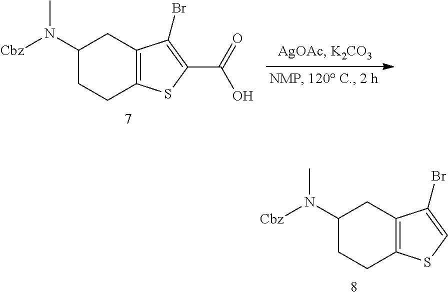 [SMILES: CN(C(=O)OCc1ccccc1)C1CCc2sc(C(=O)O)c(Br)c2C1 ; CN(C(=O)OCc1ccccc1)C1CCc2scc(Br)c2C1]

To a solution of 5-[benzyloxycarbonyl(methyl)amino]-3-bromo-4,5,6,7-tetrahydrobenzothiophene-2-carboxylic acid (7) (520 mg, 1.23 mmol, 1 eq) in NMP (6 mL) was added AgOAc (51.14 mg, 306.38 μmol, 0.25 eq) and K₂CO₃ (508.12 mg, 3.68 mmol, 3 eq). The mixture was stirred at 120° C. for 2 hrs. The residue was diluted with H₂O (30 mL) and extracted with EtOAc (10 mL*3). The combined organic layers were washed with brine (10 mL*3), dried over anhydrous sodium sulfate, filtered and concentrated under reduced pressure to give a residue. The residue was purified by flash silica gel chromatography (ISCO®; 12 g SepaFlash® Silica Flash Column, eluent 0~15% Ethyl acetate/Petroleum ether gradient @24 mL/min). Compound benzyl N-(3-bromo-4,5,6,7-tetrahydrobenzothiophen-5-yl)-N-methyl-carbamate (8) (0.3 g, 788.85 μmol, 64.37% yield) was obtained as a yellow oil. ¹H NMR (400 MHz, CHLOROFORM-d) δ=−7.48-7.29 (m, 5H), 7.08 (s, 1H), 5.18 (s, 2H), −4.66-4.26 (m, 1H), −3.05-2.83 (m, 5H), −2.81-2.65 (m, 1H), −2.63-2.45 (m, 1H), −2.03-1.85 (m, 2H).

### Procedure for preparation of 3-bromo-N-methyl-4,5,6,7-tetrahydrobenzothiophen-5-amine hydrochloride (108)

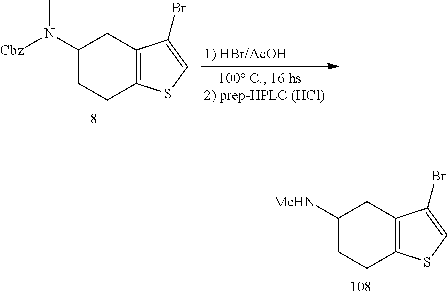 [SMILES: CN(C(=O)OCc1ccccc1)C1CCc2scc(Br)c2C1 ; CNC1CCc2scc(Br)c2C1]

To a solution of benzyl N-(3-bromo-4,5,6,7-tetrahydrobenzothiophen-5-yl)-N-methyl-carbamate (8) (0.1 g, 262.95 μmol, 1 eq) was added HBr in AcOH (2.98 g, 14.73 mmol, 2 mL, 33%, 56.03 eq). The mixture was stirred at 100° C. for 16 hours. The reaction mixture was concentrated under reduced pressure to remove solvent. Then sodium bicarbonate aq. sat. solution added (pH 8). The reaction mixture was concentrated under reduced pressure to get the residue. The residue was purified by pre-HPLC (column: Phenomenex luna C18 150*25 mm*10 μm; mobile phase: [water (HCl)-ACN]; gradient:1%-30% B over 10 min) Compound 3-bromo-N-methyl-4,5,6,7-tetrahydrobenzothiophen-5-amine hydrochloride (108) (12.88 mg, 49.18 μmol, 18.70% yield) was obtained as a white solid. ¹H NMR (400 MHz, METHANOL-d₄) δ=7.33 (s, 1H), 3.63-3.50 (m, 1H), 3.10 (br dd, J=4.6, 15.9 Hz, 1H), 3.03-2.89 (m, 2H), 2.82 (s, 3H), 2.63-2.50 (m, 1H), 2.33 (br dd, J=3.3, 8.7 Hz, 1H), 2.04-1.88 (m, 1H). LCMS: m/z [M+H]⁺=246.1.

### Synthetic scheme for preparation of 3-fluoro-N, 6-dimethyl-5,7-dihydro-4H-benzothiophen-6-amine hydrochloride (109)

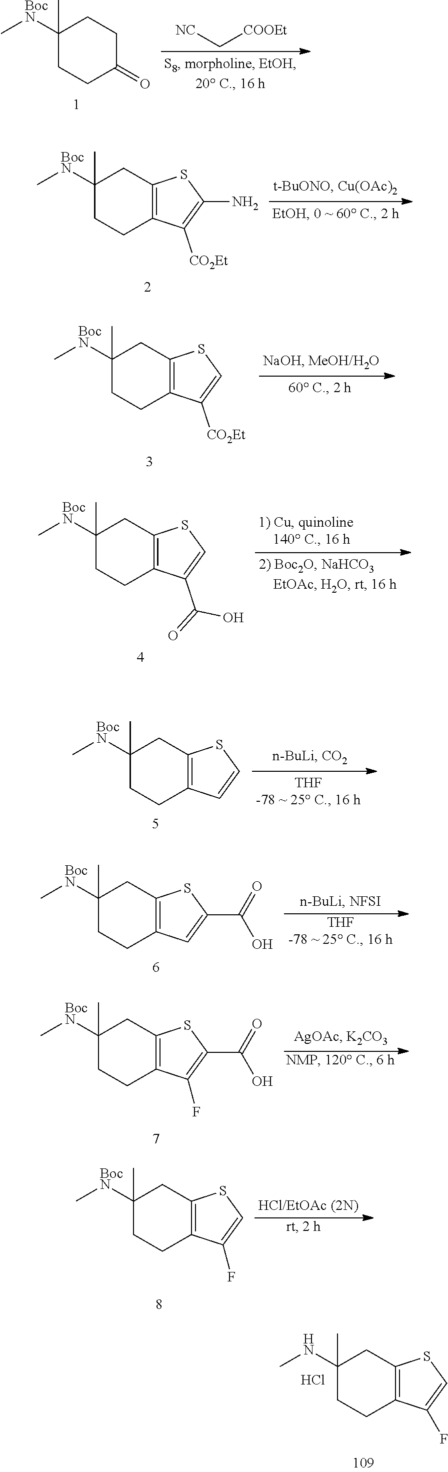

### Procedure for preparation of give ethyl 2-amino-6-[tert-butoxycarbonyl(methyl)amino]-6-methyl-5,7-dihydro-4H-benzothiophene-3-carboxylate (2)

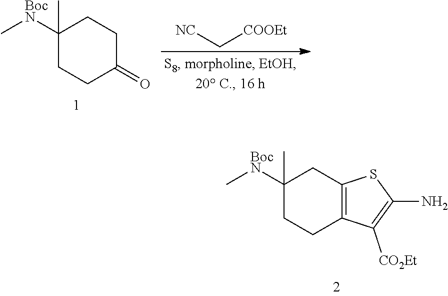 [SMILES: CN(C(=O)OC(C)(C)C)C1(C)CCC(=O)CC1 ; CCOC(=O)CC#N ; CCOC(=O)c1c(N)sc2c1CCC(C)(N(C)C(=O)OC(C)(C)C)C2]

To a solution of tert-butyl N-methyl-N-(1-methyl-4-oxo-cyclohexyl)carbamate (1) (5 g, 20.72 mmol, 1 eq) and ethyl 2-cyanoacetate (2.81 g, 24.86 mmol, 2.65 mL, 1.2 eq) in EtOH (80 mL) was added morpholine (2.17 g, 24.86 mmol, 2.19 mL, 1.2 eq) and S8 (1.01 g, 31.50 mmol, 1.52 eq). The mixture was stirred at 20° C. for 16 hr. The reaction mixture was concentrated under reduced pressure to remove EtOH. The residue was purified by flash silica gel chromatography (ISCO®; 40 g SepaFlash® Silica Flash Column, eluent 0-30% Ethyl acetate/Petroleum ether gradient @40 mL/min) to give ethyl 2-amino-6-[tert-butoxycarbonyl(methyl)amino]-6-methyl-5,7-dihydro-4H-benzothiophene-3-carboxylate (2) (6.5 g, 17.64 mmol, 85.14% yield) was obtained as a brown oil. LCMS: m/z [M+H]⁺=369.0

### Procedure for preparation of ethyl 6-[tert-butoxycarbonyl(methyl)amino]-6-methyl-5,7-dihydro-4H-benzothiophene-3-carboxylate (3)

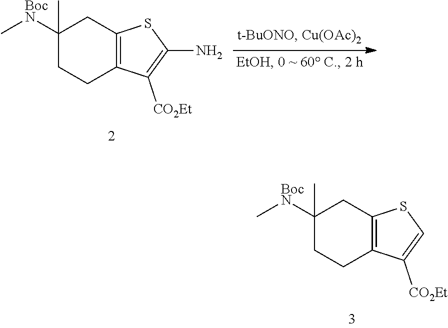 [SMILES: CCOC(=O)c1c(N)sc2c1CCC(C)(N(C)C(=O)OC(C)(C)C)C2 ; CCOC(=O)c1csc2c1CCC(C)(N(C)C(=O)OC(C)(C)C)C2]

To a solution of ethyl 2-amino-6-[tert-butoxycarbonyl(methyl)amino]-6-methyl-5,7-dihydro-4H-benzothiophene-3-carboxylate (2) (6.5 g, 17.64 mmol, 1 eq) in EtOH (70 mL) was added Cu(OAc)₂ (3.84 g, 21.17 mmol, 1.2 eq) then was added t-BuONO (2.18 g, 21.17 mmol, 2.52 mL, 1.2 eq) at 0° C. under N₂. The mixture was stirred at 60° C. for 2 hr. The reaction mixture was filtered and concentrated under reduced pressure to give a residue. The residue was purified by flash silica gel chromatography (ISCO®; 40 g SepaFlash® Silica Flash Column, eluent 0~20% Ethyl acetate/Petroleum ether gradient @35 mL/min) to give ethyl 6-[tert-butoxycarbonyl(methyl)amino]-6-methyl-5,7-dihydro-4H-benzothiophene-3-carboxylate (3) (3.6 g, 10.18 mmol, 57.74% yield) as a yellow oil.

### Procedure for preparation of 6-[tert-butoxycarbonyl(methyl)amino]-6-methyl-5,7-dihydro-4H-benzothiophene-3-carboxylic acid (4)

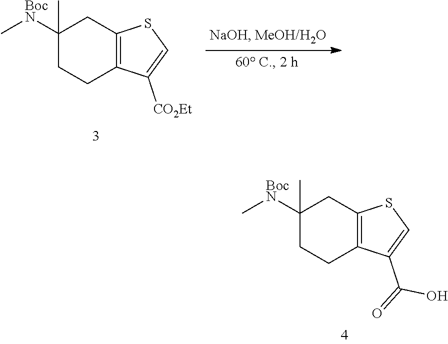 [SMILES: CCOC(=O)c1csc2c1CCC(C)(N(C)C(=O)OC(C)(C)C)C2 ; CN(C(=O)OC(C)(C)C)C1(C)CCc2c(C(=O)O)csc2C1]

To a solution of ethyl 6-[tert-butoxycarbonyl(methyl)amino]-6-methyl-5,7-dihydro-4H-benzothiophene-3-carboxylate (3) (3.6 g, 10.18 mmol, 1 eq) in MeOH (20 mL) was added NaOH (1.22 g, 30.55 mmol, 3 eq) in H₂O (20 mL). The mixture was stirred at 60° C. for 2 hr. The reaction mixture was concentrated under reduced pressure to remove MeOH. 1 M HCl aqueous solution was added into the mixture to adjust pH to 5, followed filtration, the filter cake was collected and dried to give 6-[tert-butoxycarbonyl(methyl)amino]-6-methyl-5,7-dihydro-4H-benzothiophene-3-carboxylic acid (4) (3.1 g, 9.53 mmol, 93.53% yield) as a yellow solid. ¹H NMR (400 MHz, METHANOL-d4) δ=7.96 (s, 1H), 3.38-3.33 (m, 1H), 3.01-2.86 (m, 2H), 2.78 (s, 5H), 1.85-1.73 (m, 1H), 1.46 (s, 9H), 1.43 (s, 3H)

### Procedure for preparation of tert-butyl N-methyl-N-(6-methyl-5,7-dihydro-4H-benzothiophen-6-yl)carbamate (5)

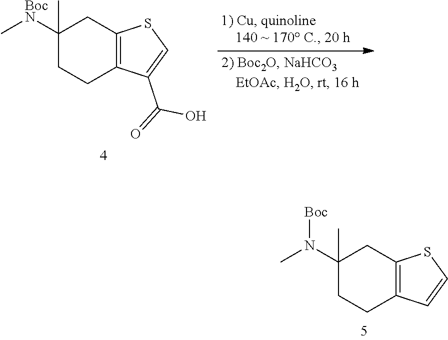 [SMILES: CN(C(=O)OC(C)(C)C)C1(C)CCc2c(C(=O)O)csc2C1 ; CN(C(=O)OC(C)(C)C)C1(C)CCc2ccsc2C1]

To a solution of 6-[tert-butoxycarbonyl(methyl)amino]-6-methyl-5,7-dihydro-4H-benzothiophene-3-carboxylic acid (4) (3.1 g, 9.53 mmol, 1 eq) in quinoline (20 mL) was added Cu (1.51 g, 23.82 mmol, 2.5 eq). The mixture was stirred at 140-170° C. for 20 hr. To the mixture was added EtOAc (50 mL) and H₂O (50 mL), then was added NaHCO₃ (2.32 g, 27.58 mmol, 5 eq) and (Boc)₂O (2.41 g, 11.03 mmol, 2.53 mL, 2 eq). The mixture was stirred at 25° C. for 16 hr. The reaction mixture was poured into H₂O (20 mL) and extracted with EtOAc (30 mL*3). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~20% Ethyl acetate/Petroleum ether gradient @40 mL/min) and then by prep-HPLC (column: Kromasil Eternity XT 250*80 mm*10 μm; mobile phase: [water(NH₄HCO₃)-ACN]; gradient:55%-85% B over 20 min) to afford tert-butyl N-methyl-N-(6-methyl-5,7-dihydro-4H-benzothiophen-6-yl)carbamate (5) (1.2 g, 4.26 mmol, 77.31% yield) as yellow oil. ¹H NMR (400 MHz, CHLOROFORM-d) δ=7.06 (d, J=5.0 Hz, 1H), 6.76 (d, J=5.0 Hz, 1H), 3.23 (d, J=16.9 Hz, 1H), 2.90 (br d, J=16.9 Hz, 1H), 2.85-2.76 (m, 4H), 2.63 (br t, J=6.1 Hz, 2H), 1.82-1.74 (m, 1H), 1.47 (s, 9H), 1.44 (s, 3H)

### Procedure for preparation of 6-[tert-butoxycarbonyl(methyl)amino]-6-methyl-5,7-dihydro-4H-benzothiophene-2-carboxylic acid (6)

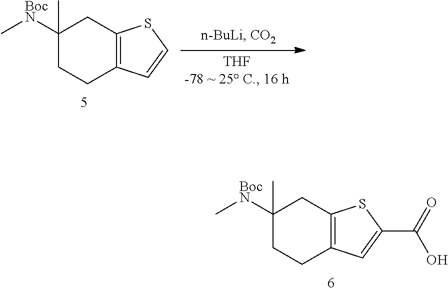

To a mixture of tert-butyl N-methyl-N-(6-methyl-5,7-dihydro-4H-benzothiophen-6-yl)carbamate (5) (1.2 g, 4.26 mmol, 1 eq) in THF (15 mL) was added n-BuLi (2.5 M, 2.56 mL, 1.5 eq) in one portion at −78° C. under N₂. The mixture was stirred at −78° C. for 0.5 hour. Then CO₂ was bubbled into the mixture and the mixture was purged with CO₂ several times. The mixture was warmed to 25° C., and stirred at 25° C. for 16 hours under CO₂. The reaction mixture was poured into NH₄Cl aq. saturated solution (50 mL), citric acid aqueous solution was added to adjust pH to 5, and the mixture was extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 25 g SepaFlash® Silica Flash Column, eluent 0~100% Ethyl acetate/Petroleum ether gradient @50 mL/min) to give 6-[tert-butoxycarbonyl(methyl)amino]-6-methyl-5,7-dihydro-4H-benzothiophene-2-carboxylic acid (6) (940 mg, 2.89 mmol, 67.74% yield) as a white solid. ¹H NMR (400 MHz, CHLOROFORM-d) δ=7.56 (s, 1H), 3.36-3.35 (m, 1H), 3.34 (br d, J=17.4 Hz, 1H), 2.93 (br d, J=17.5 Hz, 1H), 2.88-2.75 (m, 4H), 2.65 (br t, J=6.3 Hz, 2H), 1.83-1.71 (m, 1H), 1.47 (s, 9H), 1.44 (s, 3H)

### Procedure for preparation of N-methyl-3-(trifluoromethyl)-4,5,6,7-tetrahydro-2-benzothiophen-5-amine (7)

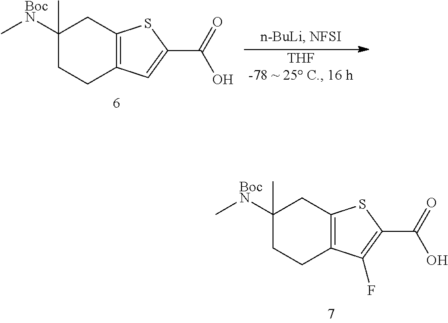

To a solution of 6-[tert-butoxycarbonyl(methyl)amino]-6-methyl-5,7-dihydro-4H-benzothiophene-2-carboxylic acid (6) (940 mg, 2.89 mmol, 1 eq) in THF (10 mL) was added dropwise n-BuLi (2.5 M, 2.89 mL, 2.5 eq) at −78° C. over 15 min. After addition, the mixture was stirred at this temperature for 0.5 hr, and then NFSI (1.37 g, 4.33 mmol, 1.5 eq) in THF (5 mL) was added dropwise at −78° C. over 15 min. The resulting mixture was warmed up to 25° C. slowly and stirred for 16 hr. The reaction mixture was poured into NH₄Cl aq. saturated solution (30 mL) and extracted with EtOAc (30 mL*3). The combined organic phase was washed with brine (10 mL), dried over anhydrous sodium sulfate, filtered and concentrated under vacuum. The crude product 6-[tert-butoxycarbonyl(methyl)amino]-3-fluoro-6-methyl-5,7-dihydro-4H-benzothiophene-2-carboxylic acid (7) was used into the next step without further purification.

### Procedure for preparation of tert-butyl N-(3-fluoro-6-methyl-5,7-dihydro-4H-benzothiophen-6-yl)-N-methyl-carbamate (8)

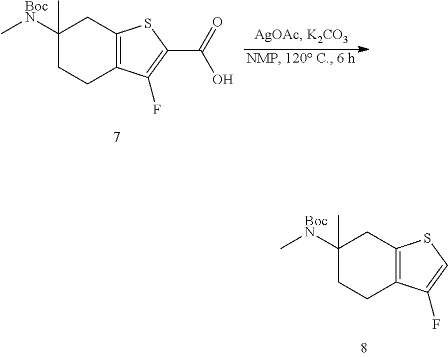 [SMILES: CN(C(=O)OC(C)(C)C)C1(C)CCc2c(sc(C(=O)O)c2F)C1 ; "NMP, 120° C., 6 h" ; CN(C(=O)OC(C)(C)C)C1(C)CCc2c(F)csc2C1]

To a solution of 6-[tert-butoxycarbonyl(methyl)amino]-3-fluoro-6-methyl-5,7-dihydro-4H-benzothiophene-2-carboxylic acid (7) (2 g, 5.82 mmol, 1 eq) in NMP (20 mL) was added AgOAc (194.42 mg, 1.16 mmol, 0.2 eq) and K₂CO₃ (2.41 g, 17.47 mmol, 3 eq). The mixture was stirred at 120° C. for 6 hrs. The reaction mixture was poured into H₂O (30 mL) and extracted with EtOAc (30 mL*3). The combined organic phases were washed with brine (10 mL), dried over anhydrous sodium sulfate, filtered and concentrated under vacuum to give a residue. The residue was purified by flash silica gel chromatography (ISCO®; 20 g SepaFlash® Silica Flash Column, eluent 0~20% Ethyl acetate/Petroleum ether gradient @50 mL/min) to give desired compound, prep-HPLC (column: Waters Xbridge Prep OBD C18 150*40 mm*10 μm; mobile phase: [water(NH₄HCO₃)-ACN]; gradient:55%-85% B over 15 min) and SFC (column: DAICEL CHIRALPAK IE (250 mm*30 mm, 10 μm); mobile phase: [CO₂-EtOH:HEX (0.1% NH₃H₂O)]; B %:10%, isocratic elution mode) to give tert-butyl N-(3-fluoro-6-methyl-5,7-dihydro-4H-benzothiophen-6-yl)-N-methyl-carbamate (8) (250 mg, 826.64 μmol, 82.50% yield) as a white solid. ¹HNMR. ¹H NMR (400 MHz, CHLOROFORM-d) 6.45 (s, 1H), 3.28-3.12 (m, 1H), 2.94-2.74 (m, 5H), 2.66-2.42 (m, 2H), 1.81-1.68 (m, 1H), 1.47 (s, 9H), 1.42 (s, 3H).

### Procedure for preparation of 3-fluoro-N, 6-dimethyl-5,7-dihydro-4H-benzothiophen-6-amine hydrochloride (109)

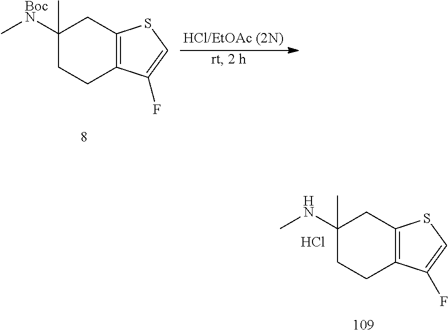 [SMILES: CN(C(=O)OC(C)(C)C)C1(C)CCc2c(F)csc2C1 ; "rt, 2 h" ; CNC1(C)CCc2c(F)csc2C1 ; Cl]

To a solution of tert-butyl N-(3-fluoro-6-methyl-5,7-dihydro-4H-benzothiophen-6-yl)-N-methyl-carbamate (8) (80 mg, 267.20 μmol, 1 eq) in EtOAc (1 mL) was added HCl/EtOAc (4 M, 1 mL, 14.97 eq). The mixture was stirred at 25° C. for 2 hr. The mixture was concentrated. The residue was triturated with EtOAc at 25° C. for 10 min. The mixture was filtered, the filter cake was collected and dried to give 3-fluoro-N, 6-dimethyl-5,7-dihydro-4H-benzothiophen-6-amine hydrochloride (109) (60.19 mg, 250.21 μmol, 93.64% yield) as a white solid. ¹H NMR (400 MHz, METHANOL-d4) δ=6.77 (s, 1H), 3.01 (s, 2H), 2.76-2.65 (m, 5H), 2.06 (t, J=6.5 Hz, 2H), 1.42 (s, 3H). LCMS: m/z [M+H]⁺=200.1

### Synthetic scheme for preparation of N-methyl-2-(trifluoromethyl)-6,7-dihydro-5H-thieno[3,2-b]pyran-6-amine hydrochloride (110)

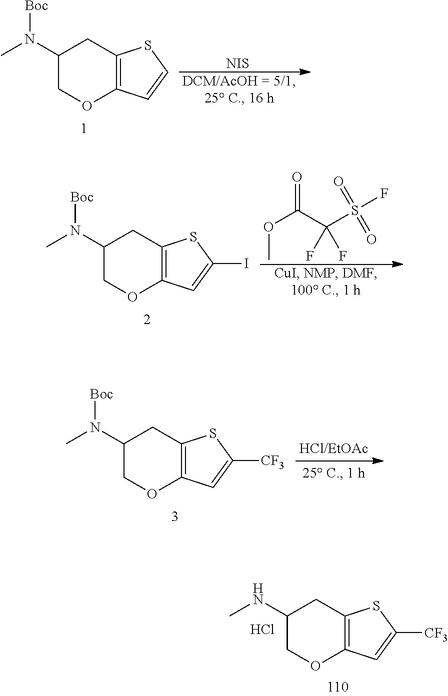

### Procedure for preparation of tert-butyl N-(2-iodo-6,7-dihydro-5H-thieno[3,2-b]pyran-6-yl)-N-methyl-carbamate (2)

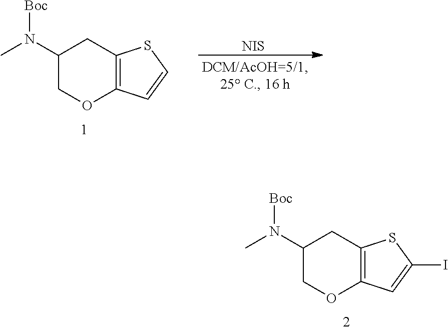 [SMILES: CN(C(=O)OC(C)(C)C)C1COc2ccsc2C1 ; "NIS" ; CN(C(=O)OC(C)(C)C)C1COc2cc(I)sc2C1]

To a solution of tert-butyl N-(6,7-dihydro-5H-thieno[3,2-b]pyran-6-yl)-N-methyl-carbamate (1) (500 mg, 1.86 mmol, 1 eq) in DCM (5 mL) was added NIS (417.63 mg, 1.86 mmol, 1 eq) and AcOH (1 mL). The mixture was stirred at 25° C. for 16 hr. The reaction mixture was quenched by addition aq. sat. NaHCO₃ (10 mL) and Na₂SO₃ (10 mL),and extracted with EtOAc (10 mL*2). The combined organic layers were washed with aq. sat. NaCl (10 mL), dried over Na₂SO₄, filtered and concentrated under reduced pressure to give a residue. The residue was purified by prep-HPLC (neutral condition: Waters Xbridge Prep OBD C18 150*40 mm*10 μm; mobile phase: [water (NH₄HCO₃)-ACN]; gradient:50%-80% B over 15 min). Compound tert-butyl N-(2-iodo-6,7-dihydro-5H-thieno[3,2-b]pyran-6-yl)-N-methyl-carbamate (2) (50 mg, 126.50 μmol, 6.81% yield) was obtained as a white solid. LCMS: m/z [M+H-tBu]⁺=339.8

### Procedure for preparation of tert-butyl N-methyl-N-[2-(trifluoromethyl)-6,7-dihydro-5H-thieno[3,2-b]pyran-6-yl]carbamate (3)

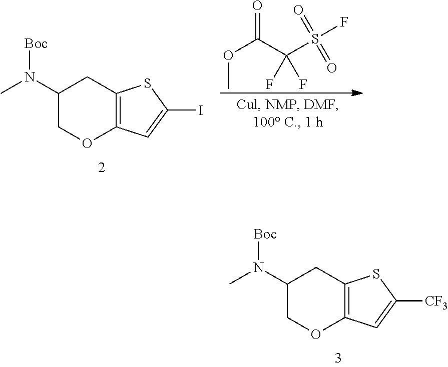

To a solution of tert-butyl N-(2-iodo-6,7-dihydro-5H-thieno[3,2-b]pyran-6-yl)-N-methyl-carbamate (2) (50 mg, 126.50 μmol, 1 eq) in DMF (1 mL) was added copper (I) iodide (24.09 mg, 126.50 μmol, 1 eq) and NMP (62.70 mg, 632.50 μmol, 61.47 μL, 5 eq). To the mixture was added methyl 2,2-difluoro-2-fluorosulfonyl-acetate (121.51 mg, 632.50 μmol, 80.47 μL, 5 eq). The mixture was stirred at 100° C. for 1 h. The reaction mixture was quenched by addition H₂O (10 mL) at 25° C., extracted with EtOAc (10 mL*2). The combined organic layers were washed with aq. sat. NaCl (15 mL*2), dried over Na₂SO₄, filtered and concentrated under reduced pressure to give a residue. The residue was purified by prep-HPLC (neutral condition: Waters Xbridge 150*25 mm*5 μm; mobile phase: [water (ammonia hydroxide v/v)-ACN]; gradient:58%-88% B).Compound tert-butyl N-methyl-N-[2-(trifluoromethyl)-6,7-dihydro-5H-thieno[3,2-b]pyran-6-yl]carbamate (3) (27 mg, 50.39 μmol, 39.84% yield) was obtained as a white solid. LCMS: m/z [M+H-Boc]⁺=237.8.

### Procedure for preparation of N-methyl-2-(trifluoromethyl)-6,7-dihydro-5H-thieno[3,2-b]pyran-6-amine hydrochloride (110)

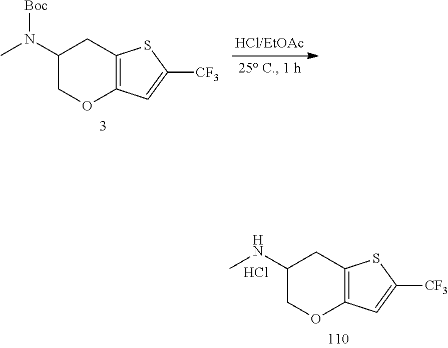 [SMILES: CN(C(=O)OC(C)(C)C)C1COc2cc(C(F)(F)F)sc2C1 ; CNC1COc2cc(C(F)(F)F)sc2C1 ; Cl]

To a solution of tert-butyl N-methyl-N-[2-(trifluoromethyl)-6,7-dihydro-5H-thieno[3,2-b]pyran-6-yl]carbamate (3) (27 mg, 80.03 μmol, 1 eq) in EtOAc (2 mL) was added HCl/EtOAc (4 M, 4 mL). The mixture was stirred at 25° C. for 1 hr. The reaction mixture was concentrated under reduced pressure to remove EtOAc. The product was triturated with EtOAc (2 mL) at 25° C. for 10 min. The mixture was filtered, the filter cake was dried. Compound N-methyl-2-(trifluoromethyl)-6,7-dihydro-5H-thieno[3,2-b]pyran-6-amine hydrochloride (110) (4.77 mg, 17.43 μmol, 21.78% yield) was obtained as a white solid. ¹H NMR (400 MHz, METHANOL-d₄) δ=7.18 (s, 1H), 4.64-4.48 (m, 1H), 4.28 (br d, J=12.9 Hz, 1H), 3.87 (br s, 1H), 3.46-3.38 (m, 1H), 3.16-3.03 (m, 1H), 2.83 (s, 3H). LCMS: m/z [M+H]⁺=238.1.

### Synthetic scheme for preparation of 6-methyl-5,7-dihydro-4H-benzothiophen-6-amine hydrochloride enantiomer 1 (111) and enantiomer 2 (112)

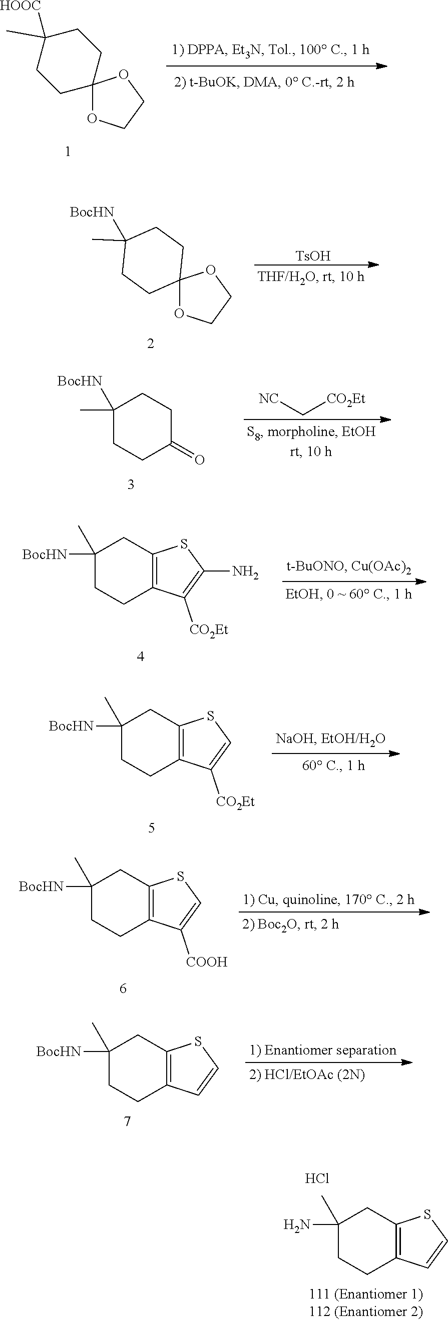

### Procedure for preparation of tert-butyl N-(8-methyl-1,4-dioxaspiro[4.5]decan-8-yl)carbamate (2)

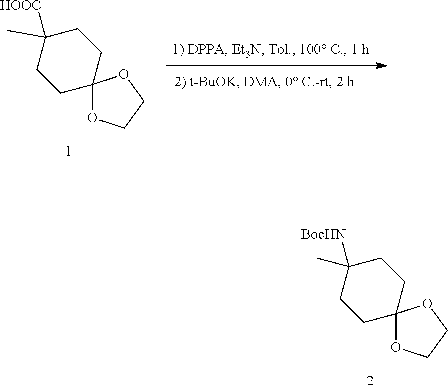

To a solution 8-methyl-1,4-dioxaspiro[4.5]decane-8-carboxylic acid (1) (10 g, 49.94 mmol, 1 eq) of in Tol. (150 mL) was added DPPA (16.49 g, 59.93 mmol, 12.94 mL, 1.2 eq) and TEA (6.06 g, 59.93 mmol, 8.34 mL, 1.2 eq). The mixture was stirred at 100° C. for 1 hr. The mixture was cooled on an ice bath, sat. NaHCO₃ (200 mL) was added to the mixture, extracted with EtOAc (200 mL*2). The combined organic layers were washed with brine (100 mL), dried over anhydrous Na₂SO₄, filtered and concentrated under reduced pressure to give a residue. The residue was dissolved in DMA (90 mL) then was added tBuOK (6.72 g, 59.93 mmol, 1.2 eq) at 0° C., then was stirred at 20° C. for 2 hr. The mixture was quenched with sat. NH₄Cl (300 mL) and extracted with EtOAc (500 mL*3). The combined organic phases were washed with brine (200 mL*3), dried over Na₂SO₄, filtered and concentrated at reduced pressure to give a residue. The residue was purified by silica gel chromatography (100-200 mesh silica gel, Petroleum ether/Ethyl acetate=1/0~10/1, Rf=0.5). Compound tert-butyl N-(8-methyl-1,4-dioxaspiro[4.5]decan-8-yl)carbamate (2) (12 g, 44.22 mmol, 88.55% yield) was obtained as a white solid. ¹H NMR (400 MHz, CHLOROFORM-d) δ=3.96-3.87 (m, 4H), 2.05-1.98 (m, 2H), 1.72-1.54 (m, 6H), 1.40 (s, 9H), 1.31 (s, 3H)

### Procedure for preparation of tert-butyl N-(1-methyl-4-oxo-cyclohexyl)carbamate (3)

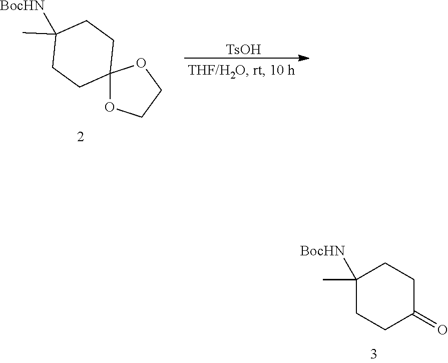 [SMILES: CC1(NC(=O)OC(C)(C)C)CCC2(CC1)OCCO2 ; "TsOH" ; CC1(NC(=O)OC(C)(C)C)CCC(=O)CC1]

To a solution of tert-butyl N-(8-methyl-1,4-dioxaspiro[4.5]decan-8-yl)carbamate (2) (12 g, 44.22 mmol, 1 eq) in THF (200 mL) and H₂O (200 mL) was added TsOH (15.23 g, 88.45 mmol, 2 eq). The mixture was stirred at 25° C. for 10 hr. The reaction mixture was quenched by addition aq. NaHCO₃100 mL, extracted with EtOAc (50 mL*2). The combined organic layers were washed with sat. NaCl (25 mL*2), dried over Na₂SO₄, filtered and concentrated under reduced pressure to give a residue. The residue was purified by flash silica gel chromatography (ISCO®; 80 g SepaFlash® Silica Flash Column, eluent 0~50% Ethyl acetate/Petroleum ether gradient @80 mL/min). Compound tert-butyl N-(1-methyl-4-oxo-cyclohexyl)carbamate (3) (8.2 g, 36.08 mmol, 81.58% yield) was obtained as a white solid. ¹H NMR (400 MHz, CHLOROFORM-d) δ=4.53 (br s, 1H), 2.52-2.24 (m, 6H), 1.79 (dt, J=4.7, 12.7 Hz, 2H), 1.46 (s, 9H), 1.43 (s, 3H).

### Procedure for preparation of ethyl 2-amino-6-(tert-butoxycarbonylamino)-6-methyl-5,7-dihydro-4H-benzothiophene-3-carboxylate (4)

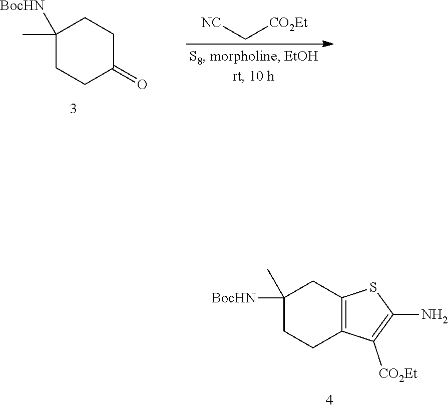 [SMILES: CC1(NC(=O)OC(C)(C)C)CCC(=O)CC1 ; CCOC(=O)CC#N ; CCOC(=O)c1c(N)sc2c1CCC(C)(NC(=O)OC(C)(C)C)C2]

To a solution of tert-butyl N-(1-methyl-4-oxo-cyclohexyl)carbamate (3) (8.2 g, 36.08 mmol, 1 eq), S (1.68 g, 52.31 mmol, 1.45 eq) and morpholine (3.14 g, 36.08 mmol, 3.17 mL, 1 eq) in EtOH (100 mL) was added ethyl 2-cyanoacetate (4.49 g, 39.68 mmol, 4.23 mL, 1.1 eq) at 0° C. The mixture was stirred at 25° C. for 10 hr. The reaction mixture was filtered and concentrated under reduced pressure to remove EtOH. The residue was purified by flash silica gel chromatography (ISCO®; 80 g SepaFlash® Silica Flash Column, eluent 0~30% Ethyl acetate/Petroleum ether gradient @80 mL/min). Compound ethyl 2-amino-6-(tert-butoxycarbonylamino)-6-methyl-5,7-dihydro-4H-benzothiophene-3-carboxylate (4) (12 g, 33.85 mmol, 93.84% yield) was obtained as a white solid. ¹H NMR (400 MHz, CHLOROFORM-d) δ=4.53 (br s, 1H), 4.34-4.22 (m, 3H), 3.46 (s, 1H), 2.85-2.62 (m, 4H), 2.28-2.17 (m, 1H), 1.44 (s, 12H), 1.37-1.30 (m, 4H). LCMS: m/z [M+H-Boc]⁺=299.1

### Procedure for preparation of ethyl 6-(tert-butoxycarbonylamino)-6-methyl-5,7-dihydro-4H-benzothiophene-3-carboxylate (5)

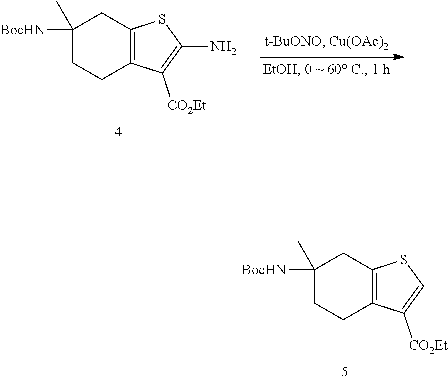 [SMILES: CCOC(=O)c1c(N)sc2c1CCC(C)(NC(=O)OC(C)(C)C)C2 ; CCOC(=O)c1csc2c1CCC(C)(NC(=O)OC(C)(C)C)C2]

To a solution of ethyl 2-amino-6-(tert-butoxycarbonylamino)-6-methyl-5,7-dihydro-4H-benzothiophene-3-carboxylate (4) (12 g, 33.85 mmol, 1 eq) and diacetoxycopper (9.22 g, 50.78 mmol, 1.5 eq) in EtOH (150 mL) was added dropwise tert-butyl nitrite (3.84 g, 37.24 mmol, 4.43 mL, 1.1 eq) at 0° C. The resulting mixture was stirred at 60° C. for 1 hr. The reaction mixture was filtered and concentrated under reduced pressure to remove EtOH. The residue was purified by flash silica gel chromatography (ISCO®; 80 g SepaFlash® Silica Flash Column, eluent 0~30% Ethyl acetate/Petroleum ether gradient @80 mL/min). Compound ethyl 6-(tert-butoxycarbonylamino)-6-methyl-5,7-dihydro-4H-benzothiophene-3-carboxylate (5) (7.5 g, 22.09 mmol, 65.26% yield) was obtained as a white solid.

### Procedure for preparation of 6-(tert-butoxycarbonylamino)-6-methyl-5,7-dihydro-4H-benzothiophene-3-carboxylic acid (6)

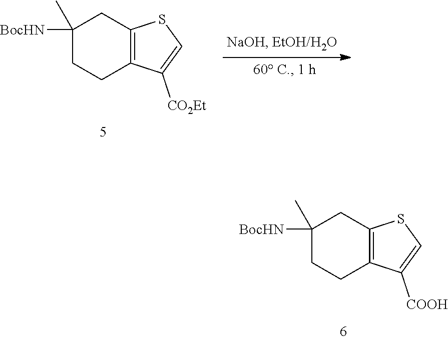 [SMILES: CCOC(=O)c1csc2c1CCC(C)(NC(=O)OC(C)(C)C)C2 ; CC1(NC(=O)OC(C)(C)C)CCc2c(C(=O)O)csc2C1]

To a solution of ethyl 6-(tert-butoxycarbonylamino)-6-methyl-5,7-dihydro-4H-benzothiophene-3-carboxylate (5) (6.5 g, 19.15 mmol, 1 eq) in EtOH (60 mL) was added NaOH (2 M, 38.30 mL, 4 eq) and H₂O (60 mL). The mixture was stirred at 60° C. for 1 hr. The reaction mixture was concentrated under reduced pressure to remove EtOH. Followed addition of aqueous HCl (1M) and pH adjusted to pH=6, then the mixture was filtered. The filter cake was collected. Compound 6-(tert-butoxycarbonylamino)-6-methyl-5,7-dihydro-4H-benzothiophene-3-carboxylic acid (6) (5 g, 16.06 mmol, 83.85% yield) was obtained as a white solid.

### Procedure for preparation of tert-butyl N-(6-methyl-5,7-dihydro-4H-benzothiophen-6-yl)carbamate (7)

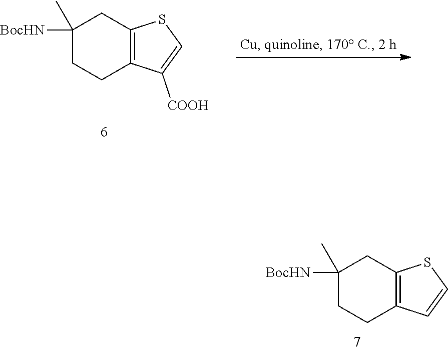 [SMILES: CC1(NC(=O)OC(C)(C)C)CCc2c(C(=O)O)csc2C1 ; CC1(NC(=O)OC(C)(C)C)CCc2ccsc2C1]

To a solution of 6-(tert-butoxycarbonylamino)-6-methyl-5,7-dihydro-4H-benzothiophene-3-carboxylic acid (6) (5 g, 16.06 mmol, 1 eq) in quinoline (50 mL) was added Cu (2.55 g, 40.14 mmol, 2.5 eq) The mixture was stirred at 170° C. for 2 hr. The mixture was filtered, then the filtrate was diluted with EtOAc (100 mL). The mixture was washed with aq. sat. NaCl (25 mL*2), dried over Na₂SO₄, filtered and concentrated under reduced pressure to give a residue. The residue was purified by flash silica gel chromatography (ISCO®; 120 g SepaFlash® Silica Flash Column, eluent 0~20% Ethyl acetate/Petroleum ether gradient @60 mL/min). Compound tert-butyl N-(6-methyl-5,7-dihydro-4H-benzothiophen-6-yl)carbamate (7) (800 mg, 2.99 mmol, 18.63% yield) was obtained as a white solid. ¹H NMR (400 MHz, CHLOROFORM-d) δ=7.09 (br d, J=5.1 Hz, 1H), 6.78 (br d, J=5.1 Hz, 1H), 4.65-4.44 (m, 1H), 2.97-2.89 (m, 2H), 2.70-2.61 (m, 2H), 2.47-2.31 (m, 2H), 1.53 (br d, J=1.6 Hz, 3H), 1.50-1.41 (m, 9H)

### Procedure for preparation of tert-butyl N-(6-methyl-5,7-dihydro-4H-benzothiophen-6-yl)carbamate enantiomer 1 (7a) and enantiomer 2 (7b)

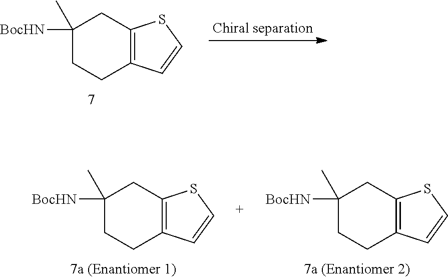 [SMILES: CC1(NC(=O)OC(C)(C)C)CCc2ccsc2C1 ; CC1(NC(=O)OC(C)(C)C)CCc2ccsc2C1 ; CC1(NC(=O)OC(C)(C)C)CCc2ccsc2C1]

Racemic tert-butyl N-(6-methyl-5,7-dihydro-4H-benzothiophen-6-yl)carbamate (7) was chirally separated using SFC (column: DAICEL CHIRALPAK IG (250 mm*50 mm, 10 μm); mobile phase: [CO₂-MeOH (0.1% NH₃H₂O)]; B %:10%, isocratic elution mode) to give enantiomer 1 (7a, 85 mg, 1^{st} peak in SFC) as a white solid and enantiomer 2 (7b, 110 mg, 2^{nd} peak in SFC) as a white solid.

### Procedure for preparation of 6-methyl-5,7-dihydro-4H-benzothiophen-6-amine hydrochloride enantiomer 1 (111)

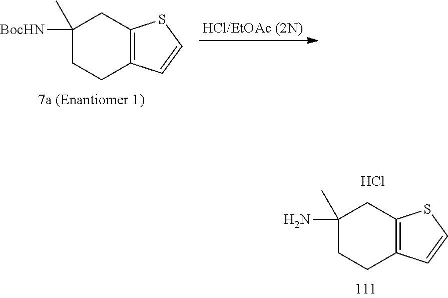 [SMILES: CC1(NC(=O)OC(C)(C)C)CCc2ccsc2C1 ; Cl ; CC1(N)CCc2ccsc2C1]

To a solution of tert-butyl N-(6-methyl-5,7-dihydro-4H-benzothiophen-6-yl)carbamate enantiomer 1 (7a) (85 mg, 317.89 μmol, 1 eq) in EtOAc (1 mL) was added HCl/EtOAc (4 M, 1 mL). The mixture was stirred at 25° C. for 1 hr. The reaction mixture was concentrated under reduced pressure to remove EtoAc. The crude product was triturated with EtOAc (2 mL) at 25° C. for 30 min. Compound 6-methyl-5,7-dihydro-4H-benzothiophen-6-amine hydrochloride enantiomer 1 (111) (58.2 mg, 285.67 μmol, 89.86% yield) was obtained as a white solid. ¹H NMR (400 MHz, METHANOL-d₄) δ=7.25 (d, J=5.1 Hz, 1H), 6.83 (d, J=5.1 Hz, 1H), 3.00 (s, 2H), 2.84-2.78 (m, 2H), 2.07-1.93 (m, 2H), 1.44 (s, 3H). LCMS: m/z [M-NH₂]⁺=152.2

### Procedure for preparation of 6-methyl-5,7-dihydro-4H-benzothiophen-6-amine hydrochloride enantiomer 2 (112)

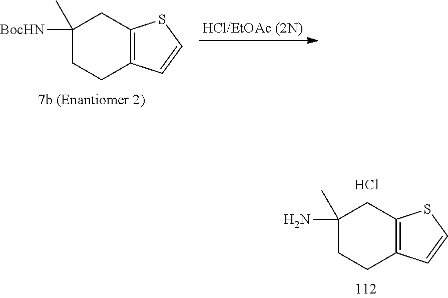 [SMILES: CC1(NC(=O)OC(C)(C)C)CCc2ccsc2C1 ; Cl ; CC1(N)CCc2ccsc2C1]

To a solution of tert-butyl N-(6-methyl-5,7-dihydro-4H-benzothiophen-6-yl)carbamate enantiomer 2 (7b) (110 mg, 411.39 μmol, 1 eq) in EtOAc (1 mL) was added HCl/EtOAc (4 M, 1 mL). The mixture was stirred at 25° C. for 1 hr. The reaction mixture was concentrated under reduced pressure to remove EtOAc. The crude product was triturated with EtOAc (2 mL) at 25° C. for 30 min. Compound 6-methyl-5,7-dihydro-4H-benzothiophen-6-amine hydrochloride enantiomer 2 (112) (63.57 mg, 380.04 μmol, 92.38% yield) was obtained as a white solid. ¹H NMR (400 MHz, METHANOL-d₄) δ=7.25 (d, J=5.1 Hz, 1H), 6.83 (d, J=5.1 Hz, 1H), 2.99 (s, 2H), 2.81 (s, 2H), 2.00 (q, J=7.0 Hz, 2H), 1.44 (s, 3H). LCMS: m/z [M-NH₂]⁺=152.2.

### Synthetic scheme for synthesis of 1-ethyl-N-methyl-4,5,6,7-tetrahydro-2-benzothiophen-5-amine hydrochloride enantiomer 1 (113) and enantiomer 2 (114), 3-ethyl-N-methyl-4,5,6,7-tetrahydro-2-benzothiophen-5-amine hydrochloride enantiomer 1 (115) and enantiomer 2 (116), 1,3-diethyl-N-methyl-4,5,6,7-tetrahydro-2-benzothiophen-5-amine hydrochloride enantiomer 1 (117) and enantiomer 2 (118)

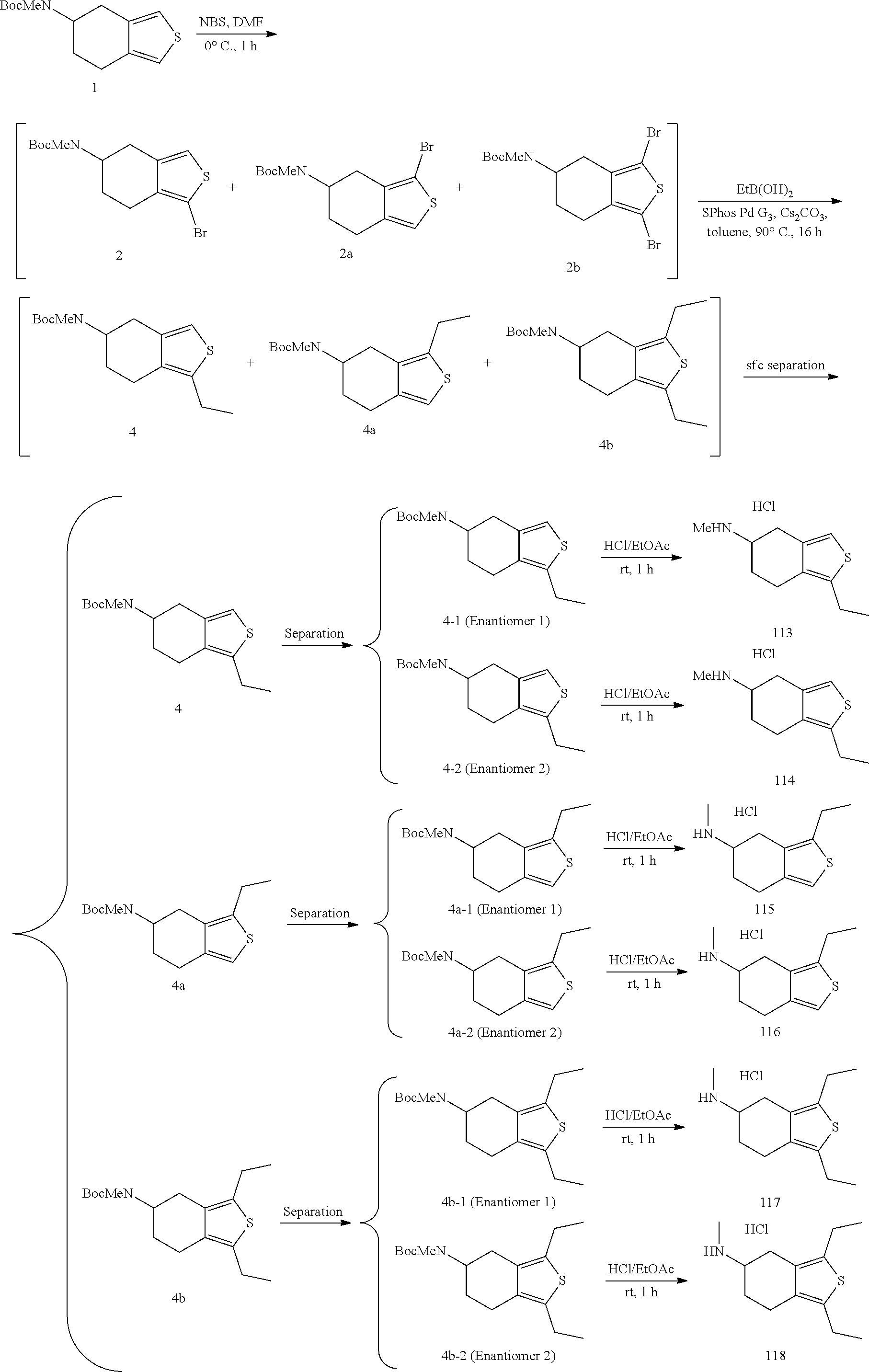

### Procedure for preparation of tert-butyl (1-bromo-4,5,6,7-tetrahydrobenzo[c]thiophen-5-yl)(methyl)carbamate(2), tert-butyl (3-bromo-4,5,6,7-tetrahydrobenzo[c]thiophen-5-yl)(methyl)carbamate (2a), tert-butyl (1,3-dibromo-4,5,6,7-tetrahydrobenzo[c]thiophen-5-yl)(methyl)carbamate(2b)

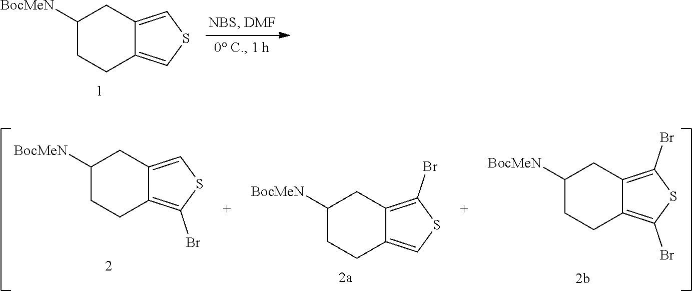

To a solution of tert-butyl N-methyl-N-(4,5,6,7-tetrahydro-2-benzothiophen-5-yl)carbamate (1) (5 g, 18.70 mmol, 1 eq) in DMF (20 mL) was added NBS (3.33 g, 18.70 mmol, 1 eq). The mixture was stirred at 0° C. for 1 hr. The reaction mixture was diluted with H₂O 20 mL and extracted with EtOAc (20 mL*3). The combined organic layers were washed with aqueous sat. NaCl (20 mL*3), dried over Na₂SO₄, filtered and concentrated under reduced pressure to give a residue. The residue was purified by column chromatography (SiO2, Petroleum ether/Ethyl acetate=1/0~10/1). Mixture of tert-butyl (1-bromo-4,5,6,7-tetrahydrobenzo[c]thiophen-5-yl)(methyl)carbamate (2), tert-butyl (3-bromo-4,5,6,7-tetrahydrobenzo[c]thiophen-5-yl)(methyl)carbamate (2a) and tert-butyl (1,3-dibromo-4,5,6,7-tetrahydrobenzo[c]thiophen-5-yl)(methyl)carbamate (2b) (5.5 g, crude) was obtained as a yellow oil.

### Procedure for preparation of tert-butyl (1-ethyl-4,5,6,7-tetrahydrobenzo[c]thiophen-5-yl)(methyl)carbamate (4), tert-butyl (3-ethyl-4,5,6,7-tetrahydrobenzo[c]thiophen-5-yl)(methyl)carbamate (4a), tert-butyl (1,3-diethyl-4,5,6,7-tetrahydrobenzo[c]thiophen-5-yl)(methyl)carbamate (4b)

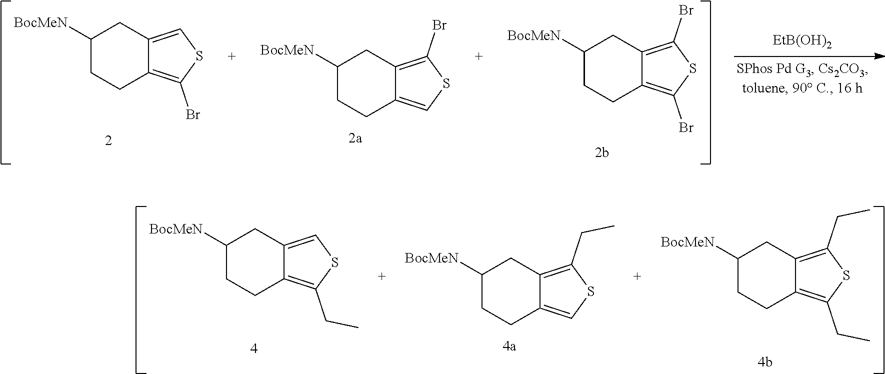

To a solution of 5 g mixture of tert-butyl (1-bromo-4,5,6,7-tetrahydrobenzo[c]thiophen-5-yl)(methyl)carbamate (2), tert-butyl (3-bromo-4,5,6,7-tetrahydrobenzo[c]thiophen-5-yl)(methyl)carbamate (2a), tert-butyl (1,3-dibromo-4,5,6,7-tetrahydrobenzo[c]thiophen-5-yl)(methyl)carbamate (2b) (14.44 mmol, 1 eq) in toluene (20 mL) was added ethylboronic acid (3.20 g, 43.32 mmol, 3 eq) Cs₂CO₃ (1.5 M, 28.88 mL, 3 eq) and SPhos Pd G3 (1.13 g, 1.44 mmol, 0.1 eq). The mixture was purged with N₂, and then stirred at 90° C. for 16 hr. The reaction mixture was diluted with H₂O (20 mL) and extracted with EtOAc (20 mL*3). The combined organic layers were washed with brine (10 mL*3), dried over Na₂SO₄, filtered and concentrated under reduced pressure to give a residue. The residue was purified by prep-HPLC (FA condition:column: Phenomenex luna C18 (250*70 mm, 10 μm); mobile phase: [water(FA)-ACN]; gradient:55%-85% B over 20 min) to give 3 g mixture of compound (4) and (4a) as white solid and 440 mg of compound (4b) as a white solid. 1 g of the mixture of compound (4) and (4a) was further separated by SFC (column: DAICEL CHIRALPAK AD (250 mm*30 mm, 10 μm); mobile phase: [CO₂-EtOH (0.1% NH₃H₂O)]; B %:10%, isocratic elution mode) to give 500 mg of compound (4) (peak 2) and 260 mg of compound (4a) (peak 1) as white solid. LCMS (4 and 4a): m/z [M+H-tBu]⁺=239.8, LCMS (4b): m/z [M+H-tBu]⁺=269.9

### Procedure for preparation of tert-butyl (1-ethyl-4,5,6,7-tetrahydrobenzo[c]thiophen-5-yl)(methyl)carbamate enantiomer 1 (4-1), and enantiomer 2 (4-2)

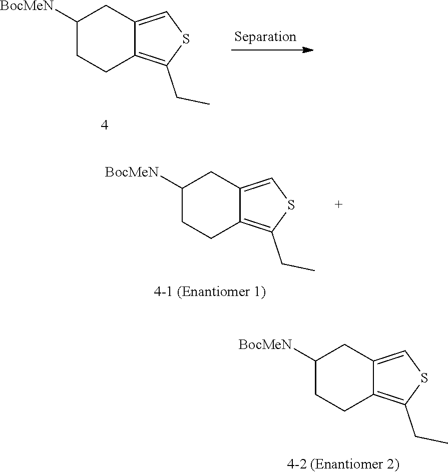

The 500 mg mixture of 4 was separated by SFC (column: DAICEL CHIRALPAK AY (250 mm*50 mm, 10 μm); mobile phase: [CO₂-EtOH (0.1% NH₃H₂O)]; B %:10%, isocratic elution mode) to yield compound tert-butyl (1-ethyl-4,5,6,7-tetrahydrobenzo[c]thiophen-5-yl)(methyl)carbamate enantiomer 1 (4-1) (150 mg, 507.72 μmol, 3.49% yield) as a white solid and tert-butyl (1-ethyl-4,5,6,7-tetrahydrobenzo[c]thiophen-5-yl)(methyl)carbamate enantiomer 2 (4-2) (160 mg, 541.57 μmol, 3.72% yield) as a white solid.

### Procedure for preparation of tert-butyl (3-ethyl-4,5,6,7-tetrahydrobenzo[c]thiophen-5-yl)(methyl)carbamate enantiomer 1 (4a-1), and enantiomer 2 (4a-2)

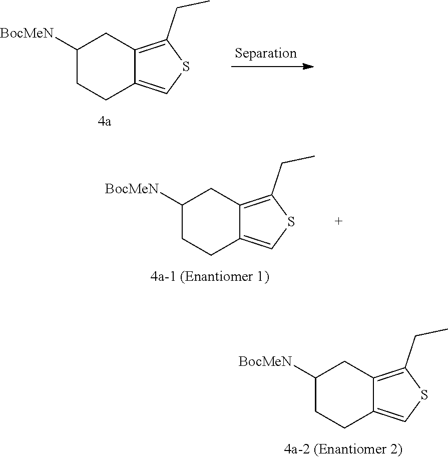

The 260 mg of racemic (4a) was separated by SFC (column: DAICEL CHIRALPAK AY (250 mm*50 mm, 1 0 m); mobile phase: [CO₂-EtOH (0.1% NH₃H₂O)]; B %:10%, isocratic elution mode) to yield compound tert-butyl (3-ethyl-4,5,6,7-tetrahydrobenzo[c]thiophen-5-yl)(methyl)carbamate enantiomer 1 (4a-1) (100 mg, 338.48 μmol, 2.33% yield) as a white solid and tert-butyl (3-ethyl-4,5,6,7-tetrahydrobenzo[c]thiophen-5-yl)(methyl)carbamate enantiomer 2 (4a-2) (140 mg, 473.87 μmol, 3.26% yield) as a white solid.

### Procedure for preparation of tert-butyl (1,3-diethyl-4,5,6,7-tetrahydrobenzo[c]thiophen-5-yl)(methyl)carbamate enantiomer 1 (4b-1) and enantiomer 2 (4b-2)

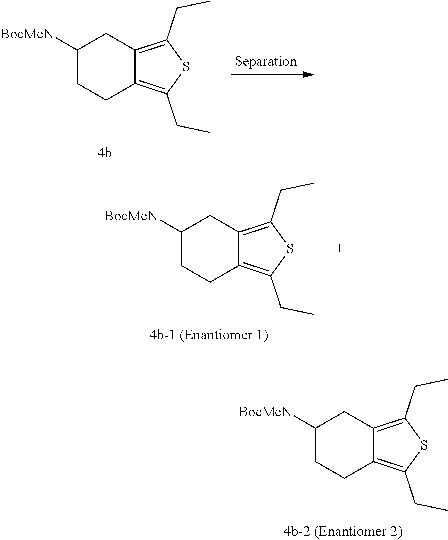

Racemic 4b was separated by SFC (column: DAICEL CHIRALPAK IG (250 mm*50 mm, 10 μm); mobile phase: [CO₂-iPrOH (0.1% NH₃H₂O)]; B %:10%, isocratic elution mode) to yield tert-butyl (1,3-diethyl-4,5,6,7-tetrahydrobenzo[c]thiophen-5-yl)(methyl)carbamate enantiomer 1 (4b-1) (116 mg, 358.59 μmol, 26.36% yield) as a white solid and tert-butyl (1,3-diethyl-4,5,6,7-tetrahydrobenzo[c]thiophen-5-yl)(methyl)carbamate enantiomer 2 (4b-2) (95 mg, 293.67 μmol, 21.59% yield) as a white solid.

### Procedure for preparation of 1-ethyl-N-methyl-4,5,6,7-tetrahydro-2-benzothiophen-5-amine hydrochloride enantiomer 1 (113)

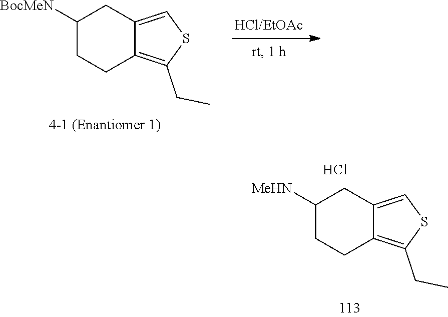

To a solution of tert-butyl (1-ethyl-4,5,6,7-tetrahydrobenzo[c]thiophen-5-yl)(methyl)carbamate enantiomer 1 (4-1) (150 mg, 507.72 μmol, 1 eq) in EtOAc (2 mL) was added HCl/EtOAc (4 M, 4 mL, 31.51 eq). The mixture was stirred at 25° C. for 1 hr. The reaction mixture was concentrated under reduced pressure to remove EtOAc. The crude product was triturated with EtOAc at 25° C. for 30 min. Compound 1-ethyl-N-methyl-4,5,6,7-tetrahydro-2-benzothiophen-5-amine hydrochloride enantiomer 1 (113) (87.05 mg, 445.67 μmol, 87.78% yield) was obtained as a white solid. ¹H NMR (400 MHz, METHANOL-d₄) δ 6.88 (s, 1H), 3.42 (ddt, J=2.8, 5.0, 10.2 Hz, 1H), 3.25 (br dd, J=4.9, 15.6 Hz, 1H), 2.86 (td, J=5.1, 16.6 Hz, 1H), 2.80-2.59 (m, 7H), 2.34-2.24 (m, 1H), 1.86 (dtd, J=5.7, 10.6, 12.9 Hz, 1H), 1.24 (t, J=7.5 Hz, 3H), LCMS: m/z [M+H]⁺=196.2

### Procedure for preparation of 1-ethyl-N-methyl-4,5,6,7-tetrahydro-2-benzothiophen-5-amine hydrochloride enantiomer 2 (114)

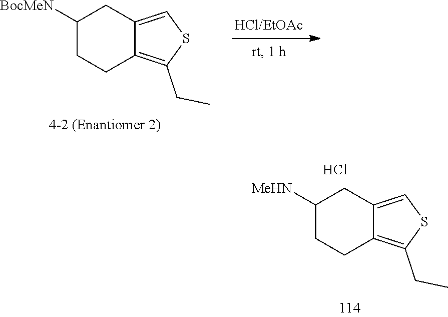

To a solution of tert-butyl (1-ethyl-4,5,6,7-tetrahydrobenzo[c]thiophen-5-yl)(methyl)carbamate enantiomer 2 (4-2) (160 mg, 541.57 μmol, 1 eq) in EtOAc (2 mL) was added HCl/EtOAc (4 M, 4.27 mL, 31.51 eq). The mixture was stirred at 25° C. for 1 hr. The reaction mixture was concentrated under reduced pressure to remove EtOAc. Compound 1-ethyl-N-methyl-4,5,6,7-tetrahydro-2-benzothiophen-5-amine hydrochloride enantiomer 2 (114) (101.29 mg, 518.58 μmol, 95.75% yield) was obtained as a white solid. ¹H NMR (400 MHz, METHANOL-d₄) δ 6.88 (s, 1H), 3.52-3.35 (m, 1H), 3.24 (dd, J=4.8, 15.4 Hz, 1H), 2.91-2.82 (m, 1H), 2.80-2.58 (m, 7H), 2.34-2.22 (m, 1H), 1.85 (dtd, J=5.7, 10.5, 12.9 Hz, 1H), 1.24 (t, J=7.5 Hz, 3H). LCMS: m/z [M+H]⁺=196.2

### Procedure for preparation of 3-ethyl-N-methyl-4,5,6,7-tetrahydro-2-benzothiophen-5-amine hydrochloride enantiomer 1 (115)

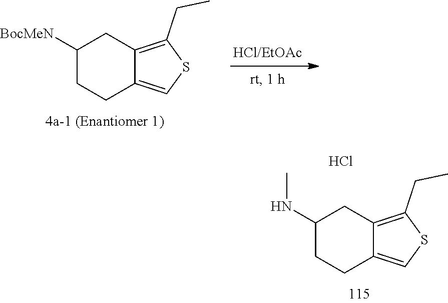

To a solution of tert-butyl (3-ethyl-4,5,6,7-tetrahydrobenzo[c]thiophen-5-yl)(methyl)carbamate enantiomer 1 (4a-1) (100 mg, 338.48 μmol, 1 eq) in EtOAc (2 mL) was added HCl/EtOAc (4 M, 4 mL). The mixture was stirred at 25° C. for 1 hr. The reaction mixture was concentrated under reduced pressure to remove EtOAc. The crude product was triturated with EtOAc at 25° C. for 30 min. Compound 3-ethyl-N-methyl-4,5,6,7-tetrahydro-2-benzothiophen-5-amine hydrochloride enantiomer 1 (115) (24.91 mg, 107.47 μmol, 31.75% yield) was obtained as a white solid. ¹H NMR (400 MHz, METHANOL-d₄) δ 6.81 (s, 1H), 3.51-3.39 (m, 1H), 3.19 (dd, J=5.1, 15.1 Hz, 1H), 2.98-2.86 (m, 1H), 2.82-2.71 (m, 6H), 2.58 (dd, J=9.8, 15.6 Hz, 1H), 2.29-2.18 (m, 1H), 1.89-1.75 (m, 1H), 1.30-1.22 (m, 3H). LCMS: m/z [M+H]⁺=196.2

### Procedure for preparation of 3-ethyl-N-methyl-4,5,6,7-tetrahydro-2-benzothiophen-5-amine hydrochloride enantiomer 2 (116)

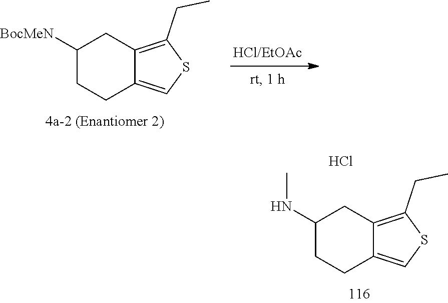

To a solution of tert-butyl (3-ethyl-4,5,6,7-tetrahydrobenzo[c]thiophen-5-yl)(methyl)carbamate (4a-2) (140 mg, 473.87 μmol, 1 eq) in EtOAc (2 mL) was added HCl/EtOAc (4 M, 4 mL). The mixture was stirred at 25° C. for 1 hr. The reaction mixture was concentrated under reduced pressure to remove EtOAc. The residue was purified by SFC (condition:column:Phenomenex-Cellulose-2 (250 mm*30 mm, 10 μm); mobile phase: [CO₂-EtOH (0.1% NH₃H₂O)]; B %:40%, isocratic elution mode), and then further purified by prep-HPLC (HCl condition:Welch Xtimate C18 150*25 mm*5 μm; mobile phase: [water(HCl)—CH₃CN]; gradient:5%-35% B over 9 min) to give Compound 3-ethyl-N-methyl-4,5,6,7-tetrahydro-2-benzothiophen-5-amine hydrochloride enantiomer 2 (116) (36.21 mg, 156.22 μmol, 32.97% yield) as a white solid. ¹H NMR (400 MHz, METHANOL-d₄) δ 6.81 (s, 1H), 3.51-3.39 (m, 1H), 3.19 (dd, J=5.1, 15.1 Hz, 1H), 2.98-2.86 (m, 1H), 2.82-2.71 (m, 6H), 2.58 (dd, J=9.8, 15.6 Hz, 1H), 2.29-2.18 (m, 1H), 1.89-1.75 (m, 1H), 1.30-1.22 (m, 3H) LCMS: m/z [M+H]⁺=196.0

### Procedure for preparation of 1,3-diethyl-N-methyl-4,5,6,7-tetrahydro-2-benzothiophen-5-amine hydrochloride enantiomer 1 (117)

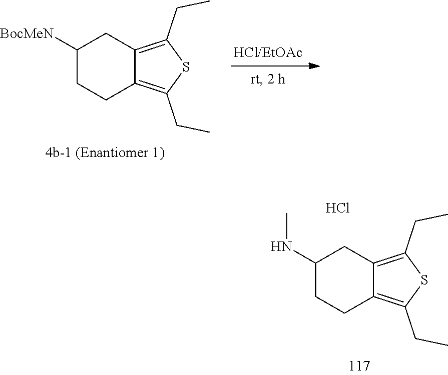

To a solution of tert-butyl (1,3-diethyl-4,5,6,7-tetrahydrobenzo[c]thiophen-5-yl)(methyl)carbamate enantiomer 1 (4b-1) (70 mg, 216.39 μmol, 1 eq) in EtOAc (2 mL) was added HCl/EtOAc (4 M, 4 mL, 73.94 eq). The mixture was stirred at 25° C. for 2 hr. The reaction mixture was concentrated under reduced pressure to remove EtOAc. The crude product was triturated with EtOAc at 25° C. for 30 min filtered and dried. Compound 1,3-diethyl-N-methyl-4,5,6,7-tetrahydro-2-benzothiophen-5-amine hydrochloride enantiomer 1 (117) (45.14 mg, 173.72 μmol, 80.28% yield) was obtained as a white solid. ¹H NMR (400 MHz, METHANOL-d₄) δ 3.40 (br d, J=2.9 Hz, 1H), 3.12 (dd, J=5.2, 15.6 Hz, 1H), 2.87-2.77 (m, 4H), 2.75-2.46 (m, 6H), 2.29-2.20 (m, 1H), 1.88-1.75 (m, 1H), 1.22 (q, J=7.4 Hz, 6H). LCMS: m/z [M+H]⁺=224.4

### Procedure for preparation of 1,3-diethyl-N-methyl-4,5,6,7-tetrahydro-2-benzothiophen-5-amine hydrochloride enantiomer 2 (118)

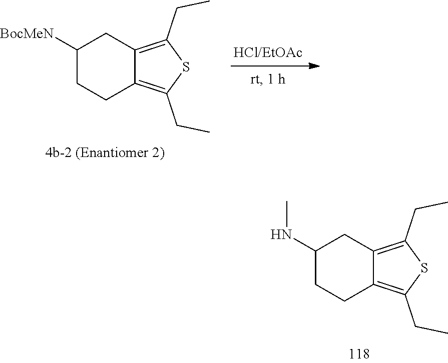

To a solution of of tert-butyl (1,3-diethyl-4,5,6,7-tetrahydrobenzo[c]thiophen-5-yl)(methyl)carbamate enantiomer 2 (4b-2) (80 mg, 247.30 μmol, 1 eq) in EtOAc (2 mL) was added HCl/EtOAc (4 M, 4.57 mL, 73.94 eq). The mixture was stirred at 25° C. for 1 hr. The reaction mixture was concentrated under reduced pressure to remove EtOAc. The crude product was triturated with EtOAc at 25° C. for 30 min. Compound 1,3-diethyl-N-methyl-4,5,6,7-tetrahydro-2-benzothiophen-5-amine hydrochloride enantiomer 2 (118) (45.14 mg, 173.72 μmol, 70.25% yield) was obtained as a white solid. ¹H NMR (400 MHz, METHANOL-d₄) δ 3.46-3.37 (m, 1H), 3.13 (dd, J=4.5, 15.5 Hz, 1H), 2.87-2.77 (m, 4H), 2.75-2.50 (m, 6H), 2.28-2.19 (m, 1H), 1.89-1.75 (m, 1H), 1.26-1.17 (m, 6H). LCMS: m/z [M+H]⁺=224.3

### Synthetic scheme for preparation of N, N, 6-trimethyl-5, 7-dihydro-4H-benzothiophen-6-amine hydrochloride enantiomer 1 (119)

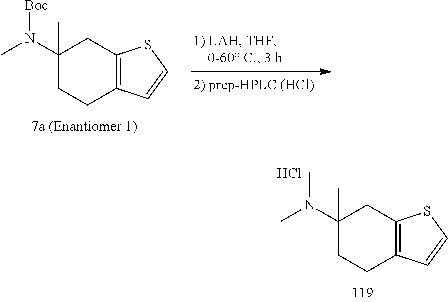 [SMILES: CN(C(=O)OC(C)(C)C)C1(C)CCc2ccsc2C1 ; CN(C)C1(C)CCc2ccsc2C1 ; Cl]

### Procedure for preparation of N, N, 6-trimethyl-5, 7-dihydro-4H-benzothiophen-6-amine hydrochloride enantiomer 1 (119)

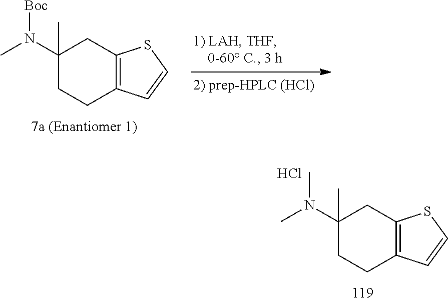 [SMILES: CN(C(=O)OC(C)(C)C)C1(C)CCc2ccsc2C1 ; CN(C)C1(C)CCc2ccsc2C1 ; Cl]

To a solution of tert-butyl N-methyl-N-(6-methyl-5, 7-dihydro-4H-benzothiophen-6-yl)carbamate enantiomer 1 (7a) (170 mg, 604 μmol, 1 eq) in THF (5 mL) was added LiAlH₄ (68.8 mg, 1.81 mmol, 3 eq) at 0° C. The resulting mixture was stirred at 60° C. for 3 hr. After the reaction mixture was cooled to 0° C., the reaction mixture was quenched by addition of 10 g of Na₂SO₄·10H₂O. After being stirred at room temperature for 0.5 h, the solid was removed by filtration. The filtrate was concentrated to dryness to give crude product. The residue was purified by prep-HPLC (column: Welch Xtimate C18 150*25 mm*5 μm; mobile phase: [water(HCl)-ACN]; gradient: 3%-33% B over 8 min) to afford N, N, 6-trimethyl-5, 7-dihydro-4H-benzothiophen-6-amine hydrochloride enantiomer 1 (119) (98.16 mg, 414.77 μmol, 68.66% yield) as a yellow solid ¹H NMR (400 MHz, METHANOL-d₄) δ 7.26 (d, J=5.3 Hz, 1H), 6.83 (d, J=5.1 Hz, 1H), 3.24-3.06 (m, 2H), 2.92 (d, J=5.9 Hz, 6H), 2.88 (t, J=5.3 Hz, 1H), 2.82-2.70 (m, 1H), 2.26-2.16 (m, 1H), 2.14-2.03 (m, 1H), 1.40 (s, 3H). LCMS: m/z [M+H]⁺=196.2

### Synthetic scheme for preparation of N,N-dimethyl-3-(trifluoromethyl)-4,5,6,7-tetrahydro-2-benzothiophen-5-amine hydrochloride enantiomer 1 (120)

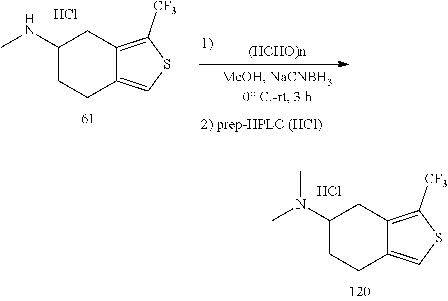 [SMILES: CNC1CCc2csc(C(F)(F)F)c2C1 ; Cl ; CN(C)C1CCc2csc(C(F)(F)F)c2C1 ; Cl]

### Procedure for preparation of N,N-dimethyl-3-(trifluoromethyl)-4,5,6,7-tetrahydro-2-benzothiophen-5-amine hydrochloride enantiomer 1 (120)

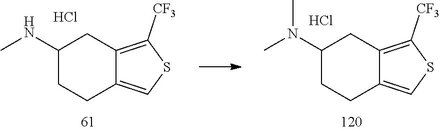 [SMILES: CNC1CCc2csc(C(F)(F)F)c2C1 ; Cl ; CN(C)C1CCc2csc(C(F)(F)F)c2C1 ; Cl]

To a solution of N-methyl-3-(trifluoromethyl)-4,5,6,7-tetrahydro-2-benzothiophen-5-amine hydrochloride enantiomer 1 (61) (80 mg, 294.41 μmol, 1.5 eq) in MeOH (2 mL) was added HCHO (5.89 mg, 196.27 μmol, 5.41 μL, 37% aq.) and the mixture was stirred at 0° C. for 0.5 hr, then sodium cyanoborohydride (18.50 mg, 294.41 μmol, 1.5 eq) was added. The mixture was stirred at 25° C. for 2.5 hr. The reaction mixture was quenched by addition aq. NH₄Cl (20 mL), extracted with EtOAc (30 mL*2). The combined organic layers were washed with aq. sat. NaCl (10 mL*2), dried over Na₂SO₄, filtered and concentrated under reduced pressure to give a residue. The residue was purified by prep-HPLC (HCl condition:column: Welch Xtimate C18 150*25 mm*5 μm; mobile phase: [water(HCl)-ACN]; B %: 8%-38%, 8 min). Compound N,N-dimethyl-3-(trifluoromethyl)-4,5,6,7-tetrahydro-2-benzothiophen-5-amine hydrochloride enantiomer 1 (120) (19 mg, 66.49 μmol, 33.88% yield) was obtained as a white solid. ¹H NMR (400 MHz, METHANOL-d₄) δ 7.38 (s, 1H), 3.71 (ddt, J=2.5, 5.3, 11.7 Hz, 1H), 3.40 (ddd, J=1.6, 3.4, 16.1 Hz, 1H), 3.15-2.93 (m, 8H), 2.87-2.76 (m, 1H), 2.37-2.26 (m, 1H), 1.99-1.85 (m, 1H). LCMS: m/z [M+H]⁺=250.2

### Synthetic scheme for preparation of N,N, 5-trimethyl-6,7-dihydro-4H-2-benzothiophen-5-amine hydrochloride enantiomer 1 (121)

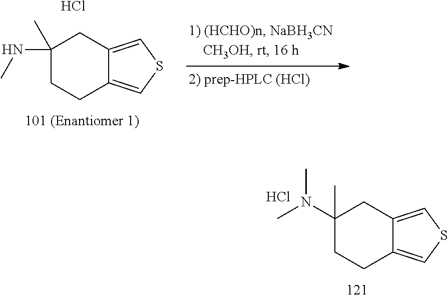

### Procedure for preparation of N,N, 5-trimethyl-6,7-dihydro-4H-2-benzothiophen-5-amine hydrochloride enantiomer 1 (121)

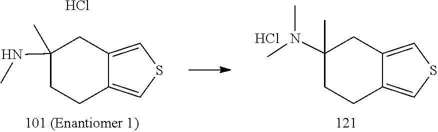

To a solution of N, 5-dimethyl-6, 7-dihydro-4H-2-benzothiophen-5-amine hydrochloride enantiomer 1 (101) (160 mg, 882 μmol, 1 eq) in MeOH (2 mL) was added HCHO (53.0 mg, 1.77 mmol, 48.6 μL, 37% aq 2 eq) and NaBH₃CN (222 mg, 3.53 mmol, 4 eq) at 0° C. The mixture was stirred at 25° C. for 16 hr. The reaction mixture was diluted with water (2 mL) extracted with EtoAc. Combined organic layer was dried over Na₂SO₄ filtered and concentrated. The residue was purified by prep-HPLC (column: Welch Xtimate C18 150*25 mm*5 μm; mobile phase: [water (HCl)-ACN]; gradient: 3%-33% B over 8 min) to afford N, N, 5-trimethyl-6, 7-dihydro-4H-2-benzothiophen-5-amine hydrochloride enantiomer 1 (121) (131.40 mg, 550.01 μmol, 62.32% yield) as a white solid. ¹H NMR (400 MHz, METHANOL-d₄) δ 7.12 (dd, J=2.8, 15.1 Hz, 2H), 3.02-3.14 (m, 2H), 3.02-2.80 (m, 8H), 2.24-2.14 (m, 1H), 2.11-2.01 (m, 1H), 1.36 (s, 3H). LCMS: m/z [M+H]⁺=196.1

### Synthetic scheme for preparation of 5-methyl-6,7-dihydro-4H-2-benzothiophen-5-amine hydrochloride enantiomer 1 (122) and enantiomer 2 (123)

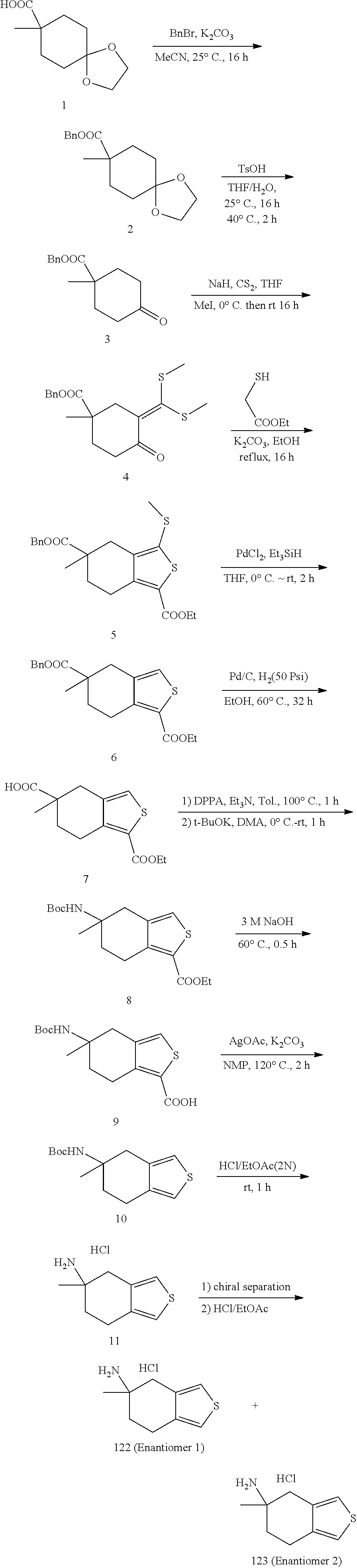

### Procedure for preparation of benzyl 8-methyl-1,4-dioxaspiro[4.5]decane-8-carboxylate (2)

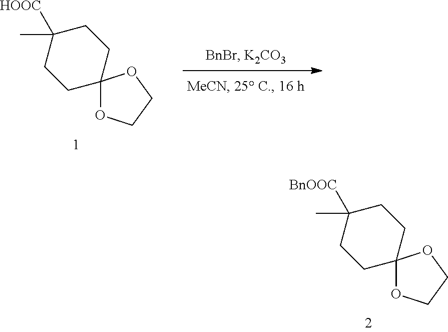

To a solution of 8-methyl-1,4-dioxaspiro[4.5]decane-8-carboxylic acid (1) (10 g, 49.94 mmol, 1 eq) and K₂CO₃ (10.35 g, 74.91 mmol, 1.5 eq) in ACN (100 mL) was added bromomethylbenzene (12.81 g, 74.91 mmol, 8.90 mL, 1.5 eq). The mixture was stirred at 25° C. for 16 hr. The mixture was quenched with saturated ammonium chloride aqueous solution (100 mL). The mixture was extracted with EtOAc (200 mL*3). The combined organic phases were washed with brine (100 mL*3), dried over Na₂SO₄, filtered and concentrated at reduced pressure to give a residue. The residue was purified by silica gel chromatography (100-200 mesh silica gel, Petroleum ether/Ethyl acetate=1/0~3/1, Rf=0.38) to afford benzyl 8-methyl-1,4-dioxaspiro[4.5]decane-8-carboxylate (2) (13 g, 44.77 mmol, 89.65% yield) as a white solid. ¹H NMR (400 MHz, Methanol-d4, 299 K) δ=7.39-7.25 (m, 5H), 5.13 (s, 2H), 3.93-3.81 (m, 4H), 2.16-2.03 (m, 2H), 1.65-1.57 (m, 2H), 1.57-1.44 (m, 4H), 1.18 (s, 3H)

### Procedure for preparation of benzyl 1-methyl-4-oxo-cyclohexanecarboxylate (3)

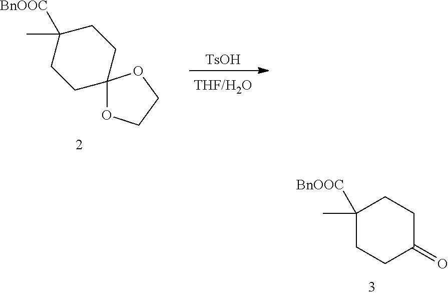

To a mixture of benzyl 8-methyl-1,4-dioxaspiro[4.5]decane-8-carboxylate (2) (13 g, 44.77 mmol, 1 eq) in THF (200 mL) and H₂O (200 mL) was added TsOH (15.42 g, 89.55 mmol, 2 eq) under N₂. The mixture was stirred at 25° C. for 16 hours. Then the mixture was stirred at 40° C. for 2 hours. The mixture was extracted with EtOAc (200 mL*3). The combined organic phases were washed with brine (100 mL*3), dried over Na₂SO₄, filtered and concentrated at reduced pressure to give a residue. The residue was purified by silica gel chromatography (100-200 mesh silica gel, Petroleum ether/Ethyl acetate=10-10/1, Rf=0.57) to afford benzyl 1-methyl-4-oxo-cyclohexanecarboxylate (3) (11 g, 44.66 mmol, 99.75% yield) as a white solid. ¹H NMR (400 MHz, Methanol-d4, 298 K) δ=7.39-7.34 (m, 5H), 5.20 (s, 2H), 2.43-2.34 (m, 4H), 2.29-2.20 (m, 2H), 1.76-1.70 (m, 2H), 1.30 (s, 3H)

### Procedure for preparation of benzyl 3-[bis (methylsulfanyl) methylene]-1-methyl-4-oxo-cyclohexanecarboxylate (4)

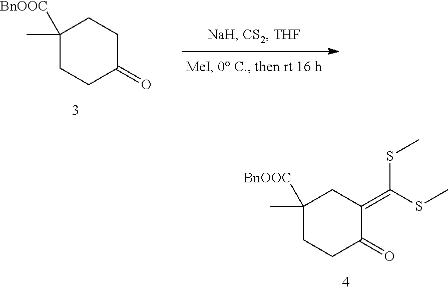

To a solution of benzyl 1-methyl-4-oxo-cyclohexanecarboxylate (3) (11 g, 44.66 mmol, 1 eq) in THF (100 mL) was added NaH (2.14 g, 53.59 mmol, 60% suspension in mineral oil, 1.2 eq) at 0° C. stirred for 0.5 h, then was added CS₂ (4.08 g, 53.59 mmol, 3.23 mL, 1.2 eq). The mixture was stirred at 0° C. for 0.5 h. The mixture was added MeI (13.95 g, 98.25 mmol, 6.12 mL, 2.2 eq) at 0° C. and stirred at 25° C. for 16 hr. The mixture was quenched with saturated ammonium chloride aqueous solution (200 mL). The mixture was extracted with EtOAc (200 mL*3). The combined organic phases were washed with brine (100 mL*3), dried over Na₂SO₄, filtered and concentrated at reduced pressure to give a residue. The residue was purified by silica gel chromatography (100-200 mesh silica gel, Petroleum ether/Ethyl acetate=1/0~10/1, Rf=0.34) to afford benzyl 3-[bis(methylsulfanyl)methylene]-1-methyl-4-oxo-cyclohexanecarboxylate (4) (11 g, 31.38 mmol, 70.27% yield) as a white solid. LCMS: m/z [M+H]⁺=351.3.

### Procedure for preparation of 5-benzyl 1-ethyl 5-methyl-3-(methylthio)-4,5,6,7-tetrahydrobenzo[c]thiophene-1,5-dicarboxylate (5)

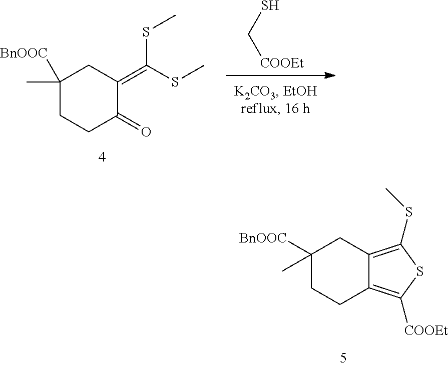

To a mixture of benzyl 3-[bis(methylsulfanyl)methylene]-1-methyl-4-oxo-cyclohexanecarboxylate (4) (11 g, 31.38 mmol, 1 eq) and K₂CO₃ (5.20 g, 37.66 mmol, 1.2 eq) in EtOH (100 mL) was added ethyl 2-sulfanylacetate (5.21 g, 43.36 mmol, 4.75 mL, 1.38 eq) under N₂. The mixture was stirred at 80° C. for 16 hours. Then the mixture cooled and was quenched with H₂O (200 mL). The mixture was extracted with EtOAc (200 mL*3). The combined organic phases were washed with brine (200 mL*3), dried over Na₂SO₄, filtered and concentrated at reduced pressure to give a residue. The residue was purified by silica gel chromatography (100-200 mesh silica gel, Petroleum ether/Ethyl acetate=1/0~10/1, Rf=0.26) to afford 5-benzyl 1-ethyl 5-methyl-3-(methylthio)-4,5,6,7-tetrahydrobenzo[c]thiophene-1,5-dicarboxylate (5) (3.7 g, 9.15 mmol, 29.14% yield) as a white solid. LCMS: m/z [M+H]⁺=405.2.

### Procedure for preparation of 5-benzyl 1-ethyl 5-methyl-4,5,6,7-tetrahydrobenzo[c]thiophene-1,5-dicarboxylate (6)

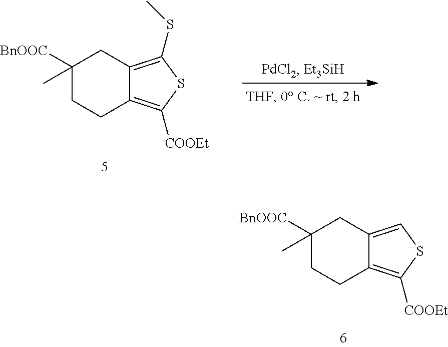

To a mixture of 5-benzyl 1-ethyl 5-methyl-3-(methylthio)-4,5,6,7-tetrahydrobenzo[c]thiophene-1,5-dicarboxylate (5) (3.7 g, 9.15 mmol, 1 eq) and PdCl₂ (162.18 mg, 914.61 μmol, 0.1 eq) in THF (80 mL) was added Et₃SiH (2.34 g, 20.12 mmol, 3.21 mL, 2.2 eq) at 0° C. under N₂. The mixture was stirred at 25° C. for 2 hours. The reaction mixture was quenched by addition H₂O (50 mL). The mixture was extracted with EtOAc (50 mL*3). The combined organic phases were washed with brine (50 mL*3), dried over Na₂SO₄, filtered and concentrated at reduced pressure to give a residue. The residue was purified by silica gel chromatography (100-200 mesh silica gel, Petroleum ether/Ethyl acetate=1/0~10/1, Rf=0.55) to afford 5-benzyl 1-ethyl 5-methyl-4,5,6,7-tetrahydrobenzo[c]thiophene-1,5-dicarboxylate (6) (3.2 g, 8.93 mmol, 97.61% yield) as a white solid. LCMS: m/z [M+H]⁺=359.2.

### Procedure for preparation of 1-ethoxycarbonyl-5-methyl-6,7-dihydro-4H-2-benzothiophene-5-carboxylic acid (7)

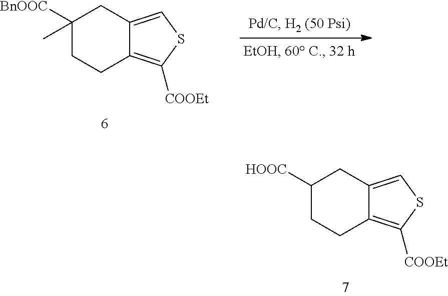

To a solution of 5-benzyl 1-ethyl 5-methyl-4,5,6,7-tetrahydrobenzo[c]thiophene-1,5-dicarboxylate (6) (3 g, 8.37 mmol, 1 eq) in EtOH (30 mL) was added Pd/C (1 g, 10% Pd) under N₂. The suspension was degassed under vacuum and purged with H₂ several times. The mixture was stirred under H₂ (50 psi) at 60° C. for 32 hours. The reaction mixture was then filtered and concentrated under reduced pressure to give a residue. The residue was purified by silica gel chromatography (100-200 mesh silica gel, Petroleum ether/Ethyl acetate=1/0~3/1, Rf=0.37) to afford 1-ethoxycarbonyl-5-methyl-6,7-dihydro-4H-2-benzothiophene-5-carboxylic acid (7) (1 g, 3.73 mmol, 44.53% yield) as a yellow oil. LCMS: m/z [M+H]⁺=269.2.

### Procedure for preparation of ethyl 5-(tert-butoxycarbonylamino)-5-methyl-6,7-dihydro-4H-2-benzothiophene-1-carboxylate (8)

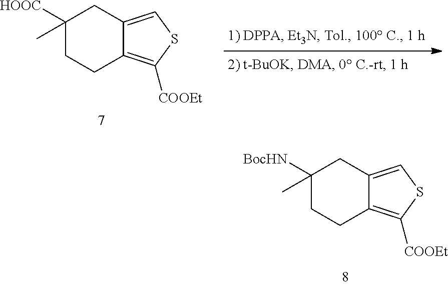 [SMILES: CCOC(=O)c1scc2c1CCC(C)(C(=O)O)C2 ; CCOC(=O)c1scc2c1CCC(C)(NC(=O)OC(C)(C)C)C2]

To a solution of 1-ethoxycarbonyl-5-methyl-6,7-dihydro-4H-2-benzothiophene-5-carboxylic acid (7) (1 g, 3.73 mmol, 1 eq) in Tol. (10 mL) was added DPPA (1.23 g, 4.47 mmol, 965.28 μL, 1.2 eq) and TEA (452.53 mg, 4.47 mmol, 622.46 μL, 1.2 eq). The mixture was stirred at 100° C. for 1 hr. The mixture was cooled on an ice bath, aq. sat. NaHCO₃ (20 mL) was added to the mixture, extracted with EtOAc (20 mL*2). The combined organic layers were washed with brine (10 mL), dried over anhydrous Na₂SO₄, filtered and concentrated under reduced pressure to give a residue. The residue was dissolved in DMA (5 mL) was added tBuOK (501.83 mg, 4.47 mmol, 1.2 eq) at 0° C., then was stirred at 20° C. for 1 hr. The reaction mixture was added to aq. sat. NH₄Cl (50 mL) at 0° C., and then diluted with H₂O (50 mL) and extracted with EtOAc (50 mL*2). The combined organic layers were washed with brine (50 mL), dried over anhydrous Na₂SO₄, filtered and concentrated under reduced pressure to give a residue. The residue was purified by silica gel chromatography (100-200 mesh silica gel, Petroleum ether/Ethyl acetate=1/0~3/1, Rf=0.54) to afford ethyl 5-(tert-butoxycarbonylamino)-5-methyl-6,7-dihydro-4H-2-benzothiophene-1-carboxylate (8) (300 mg, 883.79 μmol, 23.71% yield) as a white solid. LCMS: m/z [M+H-Boc]⁺=238.1. ¹H NMR (400 MHz, Methanol-d4298 K) δ=7.23-7.16 (m, 1H), 3.11-2.97 (m, 2H), 2.87 (ddd, J=6.3, 9.8, 18.9 Hz, 1H), 2.65 (d, J=16.1 Hz, 1H), 2.42-2.29 (m, 1H), 1.73-1.61 (m, 1H), 1.56 (s, 9H), 1.45-1.38 (m, 9H), 1.36 (s, 3H)

### Procedure for preparation of 5-(tert-butoxycarbonylamino)-5-methyl-6,7-dihydro-4H-2-benzothiophene-1-carboxylic acid (9)

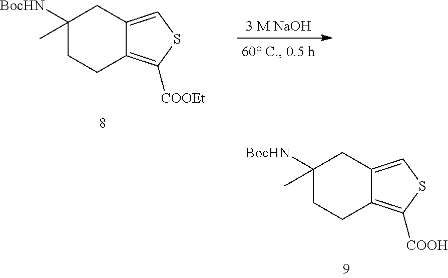 [SMILES: CCOC(=O)c1scc2c1CCC(C)(NC(=O)OC(C)(C)C)C2 ; "3 M NaOH" ; CC1(NC(=O)OC(C)(C)C)CCc2c(csc2C(=O)O)C1]

To a solution of ethyl 5-(tert-butoxycarbonylamino)-5-methyl-6,7-dihydro-4H-2-benzothiophene-1-carboxylate (8) (300 mg, 883.79 μmol, 1 eq) in MeOH (5 mL) was added NaOH (3 M, 883.79 μL, 3 eq). The mixture was stirred at 60° C. for 0.5 hr. The mixture was concentrated at reduced pressure to give a residue, water added and mixture acidified to pH=6 by HCl (1 M).Extracted with EtOAc (50 mL*2). The combined organic layers were washed with brine (50 mL), dried over anhydrous Na₂SO₄, filtered and concentrated under reduced pressure to give a residue. The residue was purified by silica gel chromatography (100-200 mesh silica gel, Petroleum ether/Ethyl acetate=1/0~3/1, Rf=0.32) to afford 5-(tert-butoxycarbonylamino)-5-methyl-6,7-dihydro-4H-2-benzothiophene-1-carboxylic acid (9) (230 mg, 738.61 μmol, 83.57% yield) as a white solid. LCMS: m/z [M+H-Boc]⁺=238.1. ¹H NMR (400 MHz, Methanol-d4, 298 K) δ=7.23 (s, 1H), 3.16-3.00 (m, 2H), 2.96-2.83 (m, 1H), 2.67 (d, J=16.1 Hz, 1H), 2.45-2.29 (m, 1H), 1.67 (ddd, J=6.2, 9.7, 13.5 Hz, 1H), 1.40 (br s, 9H), 1.37 (s, 3H)

### Procedure for preparation of tert-butyl N-(5-methyl-6,7-dihydro-4H-2-benzothiophen-5-yl)carbamate (10)

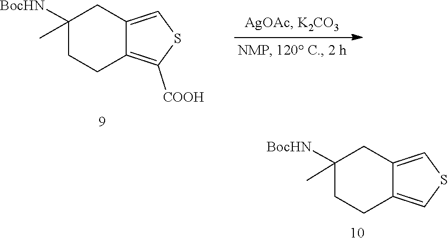 [SMILES: CC1(NC(=O)OC(C)(C)C)CCc2c(csc2C(=O)O)C1 ; CC1(NC(=O)OC(C)(C)C)CCc2cscc2C1]

To a solution of 5-(tert-butoxycarbonylamino)-5-methyl-6,7-dihydro-4H-2-benzothiophene-1-carboxylic acid (9) (230 mg, 738.61 μmol, 1 eq) in NMP (1 mL) was added AgOAc (12.33 mg, 73.86 μmol, 0.1 eq) and K₂CO₃ (306.24 mg, 2.22 mmol, 3 eq). The mixture was stirred at 120° C. for 2 hr. The mixture was diluted with water and extracted with EtOAc (30 mL*3). The combined organic phase was washed with brine (50 mL*3), dried over Na₂SO₄, filtered and concentrated at reduced pressure to give a residue. The residue was purified by silica gel chromatography (100-200 mesh silica gel, Petroleum ether/Ethyl acetate=1/0~3/1, Rf=0.74) to afford tert-butyl N-(5-methyl-6,7-dihydro-4H-2-benzothiophen-5-yl)carbamate (10) (100 mg, 373.99 μmol, 50.63% yield) as a white solid. LCMS: m/z [M+H-Boc]⁺=212.1.

### Procedure for preparation of 5-methyl-6,7-dihydro-4H-2-benzothiophen-5-amine hydrochloride (11)

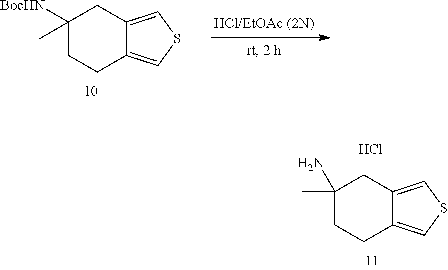 [SMILES: CC1(NC(=O)OC(C)(C)C)CCc2cscc2C1 ; Cl ; CC1(N)CCc2cscc2C1]

To a solution of tert-butyl N-(5-methyl-6,7-dihydro-4H-2-benzothiophen-5-yl) carbamate (10) (100 mg, 373.99 μmol, 1 eq) in EtOAc (1 mL) was added 4 N HCl/EtOAc (1 mL). The mixture was stirred at 25° C. for 2 h. The reaction mixture was concentrated under reduced pressure to give a residue. The crude product was purified by tirturation with EtOAc (10 mL) at 25° C. Compound 5-methyl-6,7-dihydro-4H-2-benzothiophen-5-amine hydrochloride (11) (75 mg, 368.13 μmol, 98.43% yield) was obtained as a white solid. LCMS: m/z [M+H-NH₂]⁺=151.1.

### Procedure for preparation of 5-methyl-6,7-dihydro-4H-2-benzothiophen-5-amine hydrochloride enantiomer 1 (122) and enantiomer 2 (123)

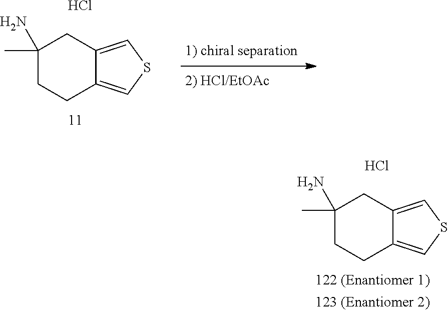

Racemic 5-methyl-6,7-dihydro-4H-2-benzothiophen-5-amine hydrochloride (11) was chirally separated by SFC (column: DAICEL CHIRALPAK AD (250 mm*50 mm, 10 μm); mobile phase: [CO₂-MeOH (0.1% NH₃H₂O)]; B %: 20%, isocratic elution mode). Each enantiomer obtained after chiral separation was treated with 4M HCl/EtOAc to yield the HCl product. Compound 5-methyl-6,7-dihydro-4H-2-benzothiophen-5-amine hydrochloride enantiomer 1 (122) (30.5 mg, 149.71 μmol, 33.39% yield) was obtained as a white solid. LCMS: m/z [M+H-NH₂]⁺=151.1. ¹H NMR (400 MHz, Methanol-d4, 296 K) δ=7.17-7.05 (m, 2H), 3.02-2.83 (m, 4H), 2.07-1.93 (m, 2H), 1.39 (s, 3H). Compound 5-methyl-6,7-dihydro-4H-2-benzothiophen-5-amine hydrochloride enantiomer 2 (123) (26.55 mg, 130.32 μmol, 29.06% yield) was obtained as a white solid. LCMS: m/z [M+H-NH2]⁺=151.1. ¹H NMR (400 MHz, Methanol-d4, 296 K) δ=7.16-7.05 (m, 2H), 3.01-2.84 (m, 4H), 2.07-1.91 (m, 2H), 1.39 (s, 3H)

### Synthetic scheme for preparation of N-cyclopropyl-4,5,6,7-tetrahydro-2-benzothiophen-5-amine hydrochloride enantiomer 1 (124) and enantiomer 2 (125)

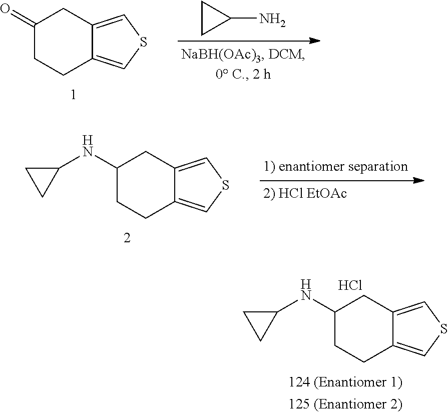

### Procedure for preparation of N-cyclopropyl-4,5,6,7-tetrahydro-2-benzothiophen-5-amine (2)

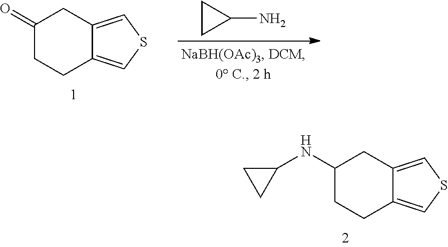

To a mixture of 6,7-dihydro-4H-2-benzothiophen-5-one (1) (500 mg, 3.28 mmol, 1 eq) and cyclopropanamine (225.06 mg, 3.94 mmol, 273.13 μL, 1.2 eq) in DCM (10 mL) was added NaBH(OAc)₃ (2.09 g, 9.85 mmol, 3 eq) in one portion at 0° C. under N₂. The mixture was stirred at 0° C. for 2 hours. The mixture was quenched with aq. saturated ammonium NH₄Cl (50 mL). The mixture was extracted with DCM (50 mL*3). The combined organic phases were washed with brine (50 mL*3), dried over Na₂SO₄, filtered and concentrated at reduced pressure to give a residue. The residue was purified by prep-TLC (SiO₂, Ethyl acetate, Rf=0.17) to afford racemic N-cyclopropyl-4,5,6,7-tetrahydro-2-benzothiophen-5-amine (2).

### Procedure for preparation of N-cyclopropyl-4,5,6,7-tetrahydro-2-benzothiophen-5-amine hydrochloride enantiomer 1 (124) and enantiomer 2 (125)

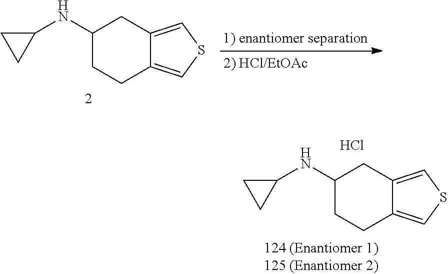 [SMILES: c1scc2c1CCC(NC1CC1)C2 ; Cl ; c1scc2c1CCC(NC1CC1)C2]

Racemic N-cyclopropyl-4,5,6,7-tetrahydro-2-benzothiophen-5-amine (2) was further separated by SFC (column: Phenomenex-Cellulose-2 (250 mm*30 mm, 10 μm); mobile phase: [0.1% NH₃H₂O EtOH]; B %: 30%-30%, 5.8 min). Each enantiomer obtained after chiral separation was treated with 4N HCl/EtOAc to obtain the corresponding hydrochloride salt. Compound N-cyclopropyl-4,5,6,7-tetrahydro-2-benzothiophen-5-amine hydrochloride enantiomer 1 (124) (109 mg, 474.39 μmol, 14.44% yield) was obtained as a white solid. LCMS: m/z [M+H]⁺=194.2. ¹H NMR (400 MHz, Methanol-d4, 298 K) δ 7.11 (br s, 1H), 7.04 (br s, 1H), 3.70-3.59 (m, 1H), 3.41 (br dd, J=5.0, 15.5 Hz, 1H), 3.05-2.96 (m, 1H), 2.91-2.78 (m, 3H), 2.40-2.31 (m, 1H), 1.97-1.84 (m, 1H), 1.04-0.90 (m, 4H). Compound N-cyclopropyl-4,5,6,7-tetrahydro-2-benzothiophen-5-amine hydrochloride enantiomer 2 (125) (155 mg, 674.59 μmol, 20.54% yield) was obtained as a white solid. LCMS: m/z [M+H]⁺=194.2. ¹H NMR (400 MHz, Methanol-d4, 298 K) δ 7.11 (br s, 1H), 7.04 (br s, 1H), 3.65 (ddt, J=2.9, 5.2, 10.6 Hz, 1H), 3.41 (dd, J=5.0, 15.7 Hz, 1H), 3.06-2.95 (m, 1H), 2.92-2.75 (m, 3H), 2.41-2.31 (m, 1H), 1.97-1.83 (m, 1H), 1.02-0.90 (m, 4H)

### Synthetic scheme for preparation of N-(3,3-difluorocyclobutyl)-4,5,6,7-tetrahydro-2-benzothiophen-5-amine hydrochloride enantiomer 1 (126) and enantiomer 2 (127)

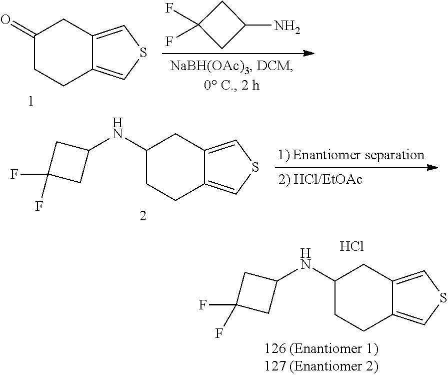

### Procedure for preparation of N-(3,3-difluorocyclobutyl)-4,5,6,7-tetrahydro-2-benzothiophen-5-amine (2)

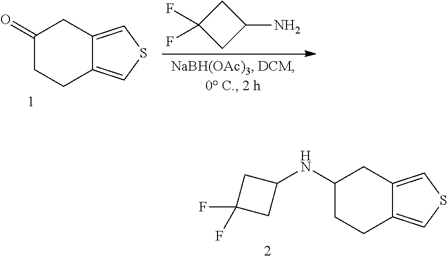

To a mixture of 6,7-dihydro-4H-2-benzothiophen-5-one (1) (500 mg, 3.28 mmol, 1 eq) and 3,3-difluorocyclobutanamine (422.18 mg, 3.94 mmol, 27.31 μL, 1.2 eq) in DCM (5 mL) was added NaBH(OAc)₃ (2.09 g, 9.85 mmol, 3 eq) in one portion at 0° C. under N₂. The mixture was stirred at 0° C. for 2 hours. The mixture was quenched with saturated ammonium NH₄Cl (50 mL). The mixture was extracted with DCM (50 mL*3). The combined organic phases were washed with brine (50 mL*3), dried over Na₂SO₄, filtered and concentrated at reduced pressure to give a residue. The residue was purified by silica gel chromatography (100-200 mesh silica gel, Petroleum ether/Ethyl acetate=1/1~0/1, Rf=0.26) to afford N-(3,3-difluorocyclobutyl)-4,5,6,7-tetrahydro-2-benzothiophen-5-amine (2)

### Procedure for preparation of N-(3,3-difluorocyclobutyl)-4,5,6,7-tetrahydro-2-benzothiophen-5-amine hydrochloride enantiomer 1 (126) and enantiomer 2 (127)

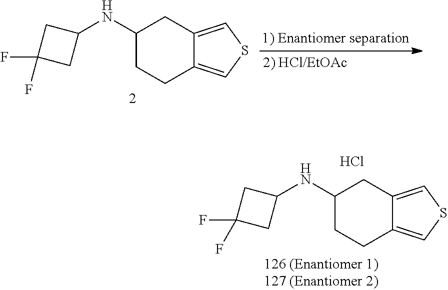 [SMILES: FC1(F)CC(NC2CCc3cscc3C2)C1 ; Cl ; FC1(F)CC(NC2CCc3cscc3C2)C1]

Racemic N-(3,3-difluorocyclobutyl)-4,5,6,7-tetrahydro-2-benzothiophen-5-amine (2) was separated to its enantiomers by SFC (column: DAICEL CHIRALPAK AD-H (250 mm*30 mm, 5 μm); mobile phase: [0.1% NH₃H₂O IPA]; B %: 15%-15%, 1.7 min). Each enantiomer was then treated with 4N HCl/EtOAc (5 mL) and stirred at 25° C. for 0.5 hour to afford the corresponding HCl salt which was collected by filtration. Compound N-(3,3-difluorocyclobutyl)-4,5,6,7-tetrahydro-2-benzothiophen-5-amine hydrochloride enantiomer 1 (126) (104.71 mg, 374.26 μmol, 11.39% yield) was obtained as an off-white solid. LCMS: m/z [M+H]⁺=244.1. ¹H NMR (400 MHz, Methanol-d4, 298 K) δ=7.11 (br s, 1H), 7.05 (br s, 1H), 4.08-3.93 (m, 1H), 3.56 (tdd, J=2.7, 10.4, 12.9 Hz, 1H), 3.34 (br s, 1H), 3.20-3.06 (m, 2H), 3.04-2.87 (m, 3H), 2.87-2.75 (m, 2H), 2.27 (br dd, J=5.0, 7.0 Hz, 1H), 1.95-1.79 (m, 1H). Compound N-(3,3-difluorocyclobutyl)-4,5,6,7-tetrahydro-2-benzothiophen-5-amine hydrochloride enantiomer 2 (127) (104.71 mg, 374.26 μmol, 11.39% yield) was obtained as a brown solid. LCMS: m/z [M+H]⁺=244.1. ¹H NMR (400 MHz, Methanol-d4, 298 K) δ=7.02-6.99 (m, 1H), 6.96-6.91 (m, 1H), 3.97-3.85 (m, 1H), 3.46 (ddt, J=2.9, 5.2, 10.4 Hz, 1H), 3.24 (d, J=5.0 Hz, 1H), 3.20-3.18 (m, 1H), 3.08-2.96 (m, 2H), 2.94-2.81 (m, 3H), 2.77-2.66 (m, 2H), 2.23-2.14 (m, 1H), 1.86-1.73 (m, 1H)

### Synthetic scheme for preparation of N-(4,5,6,7-tetrahydro-2-benzothiophen-5-vl)oxetan-3-amine enantiomer 1 (128) and enantiomer 2 (129)

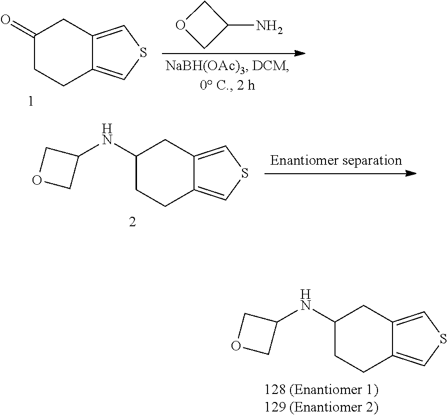

### Procedure for preparation of N-(4,5,6,7-tetrahydro-2-benzothiophen-5-yl)oxetan-3-amine (2)

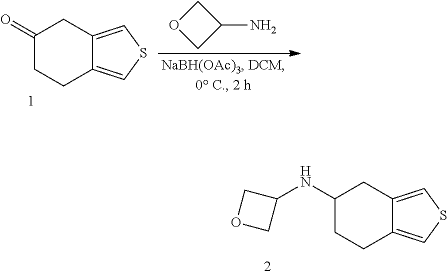

To a mixture of 6,7-dihydro-4H-2-benzothiophen-5-one (1) (500 mg, 3.28 mmol, 1 eq) and oxetan-3-amine (288.12 mg, 3.94 mmol, 27.31 μL, 1.2 eq) in DCM (10 mL) was added NaBH(OAc)₃ (2.09 g, 9.85 mmol, 3 eq) in one portion at 0° C. under N₂. The mixture was stirred at 0° C. for 2 hours. The mixture was quenched with saturated ammonium NH₄Cl (50 mL). Extracted with DCM (50 mL*3). The combined organic phases were washed with brine (50 mL*3), dried over Na₂SO₄, filtered and concentrated at reduced pressure to give a residue. The residue was purified by prep-TLC (SiO₂, Ethyl acetate, Rf=0.17) to afford N-(4,5,6,7-tetrahydro-2-benzothiophen-5-yl)oxetan-3-amine (2).

### Procedure for preparation of N-(4,5,6,7-tetrahydro-2-benzothiophen-5-yl)oxetan-3-amine enantiomer 1 (128) and enantiomer 2 (129)

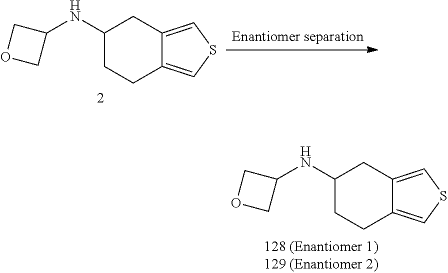 [SMILES: c1scc2c1CCC(NC1COC1)C2 ; c1scc2c1CCC(NC1COC1)C2]

Racemic N-(4,5,6,7-tetrahydro-2-benzothiophen-5-yl)oxetan-3-amine (2) was separated to enantiomers by SFC (column: DAICEL CHIRALPAK AD (250 mm*30 mm, 10 μm); mobile phase: [0.1% NH₃H₂O EtOH]; B %: 30%-30%, 4 min). Compound N-(4,5,6,7-tetrahydro-2-benzothiophen-5-yl) oxetan-3-amine enantiomer 1 (128) (174 mg, 831.31 μmol, 25.31% yield) was obtained as a white solid. LCMS: m/z [M+H]⁺=210.2. ¹H NMR (400 MHz, Methanol-d4, 298 K) δ=6.94-6.87 (m, 2H), 4.85-4.81 (m, 2H), 4.57-4.48 (m, 2H), 4.17 (quin, J=6.8 Hz, 1H), 2.98 (dd, J=4.9, 15.4 Hz, 1H), 2.93-2.81 (m, 2H), 2.65 (ddd, J=5.5, 11.1, 16.4 Hz, 1H), 2.40 (dd, J=9.8, 15.5 Hz, 1H), 1.99-1.89 (m, 1H), 1.61-1.48 (m, 1H). Compound N-(4,5,6,7-tetrahydro-2-benzothiophen-5-yl)oxetan-3-amine enantiomer 2 (129) (177 mg, 845.65 μmol, 25.74% yield) was obtained as a white solid. LCMS: m/z [M+H]⁺=210.2. ¹H NMR (400 MHz, Methanol-d4, 298 K) δ=6.94-6.88 (m, 2H), 4.85-4.81 (m, 2H), 4.57-4.50 (m, 2H), 4.17 (quin, J=6.8 Hz, 1H), 2.98 (dd, J=4.8, 15.4 Hz, 1H), 2.93-2.81 (m, 2H), 2.65 (ddd, J=5.8, 11.4, 16.8 Hz, 1H), 2.40 (dd, J=9.7, 15.5 Hz, 1H), 1.99-1.90 (m, 1H), 1.60-1.48 (m, 1H)

### Synthetic scheme for preparation of 2-fluoro-N-methyl-6,7-dihydro-5H-thieno[3,2-b]pyran-6-amine hydrochloride (130)

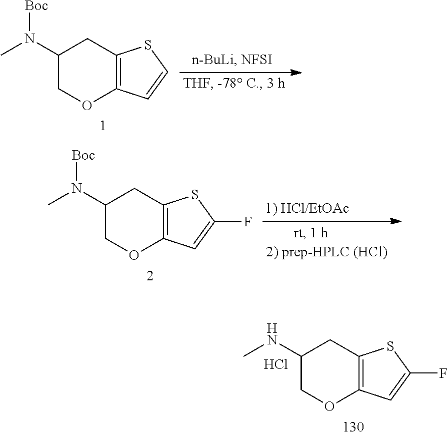 [SMILES: CN(C(=O)OC(C)(C)C)C1COc2ccsc2C1 ; CN(C(=O)OC(C)(C)C)C1COc2cc(F)sc2C1 ; CNC1COc2cc(F)sc2C1 ; Cl]

### Procedure for preparation of tert-butyl N-(2-fluoro-6,7-dihydro-5H-thieno[3,2-b]pyran-6-yl)-N-methyl-carbamate (2)

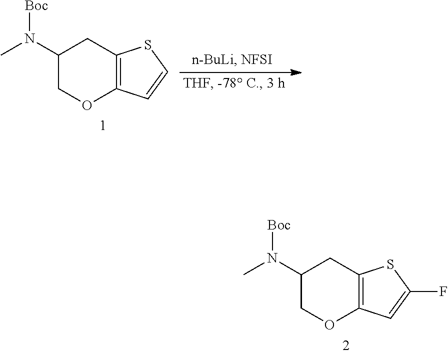 [SMILES: CN(C(=O)OC(C)(C)C)C1COc2ccsc2C1 ; CN(C(=O)OC(C)(C)C)C1COc2cc(F)sc2C1]

To the solution of tert-butyl N-(6,7-dihydro-5H-thieno[3,2-b]pyran-6-yl)-N-methyl-carbamate (1) (300 mg, 1.11 mmol, 1 eq) in THF (3 mL) was added n-BuLi (2.5 M, 1.07 mL, 2.4 eq) at −78° C. After stirring for 1 h, a solution of N-(benzenesulfonyl)-N-fluoro-benzenesulfonamide (456.57 mg, 1.45 mmol, 1.3 eq) in THF (2 mL) was added drop-wise at −78° C. and the resulting mixture was stirred for 2 h at −78° C.

The reaction mixture was quenched by addition of aq. NH₄Cl (10 mL) at 25° C. and extracted with EtOAc (10 mL*2). The combined organic layers were washed with aq. sat. NaCl (15 mL*2), dried over Na₂SO₄, filtered and concentrated under reduced pressure to give a residue. The residue was purified by prep-HPLC (neutral condition: Waters Xbridge 150*25 mm*5 μm; mobile phase: [water (ammonia hydroxide v/v)-ACN]; gradient:53%-83% B) to give tert-butyl N-(2-fluoro-6,7-dihydro-5H-thieno[3,2-b]pyran-6-yl)-N-methyl-carbamate (2) (135 mg, 469.81 μmol, 42.18% yield) as a white solid. LCMS: m/z [M+H-tBu]⁺=231.1.

### Procedure for preparation of 2-fluoro-N-methyl-6,7-dihydro-5H-thieno[3,2-b]pyran-6-amine hydrochloride (130)

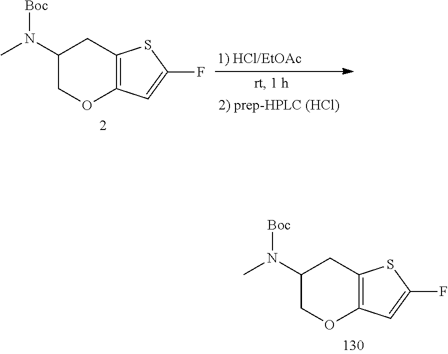 [SMILES: CN(C(=O)OC(C)(C)C)C1COc2cc(F)sc2C1 ; CN(C(=O)OC(C)(C)C)C1COc2cc(F)sc2C1]

To a solution of tert-butyl N-(2-fluoro-6,7-dihydro-5H-thieno[3,2-b]pyran-6-yl)-N-methyl-carbamate (2) (135 mg, 469.81 μmol, 1 eq) in EtOAc (2 mL) was added HCl/EtOAc (4 M, 4 mL, 34.06 eq). The mixture was stirred at 25° C. for 1 hr. The reaction mixture was then concentrated under reduced pressure to remove EtOAc. The residue was purified by prep-HPLC (HCl condition: Phenomenex luna C18 150*25 mm*10 μm; mobile phase: [water(HCl)-ACN]; gradient:1%-15% B over 10 min). Compound 2-fluoro-N-methyl-6,7-dihydro-5H-thieno[3,2-b]pyran-6-amine hydrochloride (130) (20.21 mg, 90.35 μmol, 19.23% yield) was obtained as a white solid. ¹H NMR (400 MHz, METHANOL-d₄) δ=6.25 (d, J=2.1 Hz, 1H), 4.53-4.40 (m, 1H), 4.24 (d, J=12.8 Hz, 1H), 3.81 (br d, J=1.6 Hz, 1H), 3.20 (ddd, J=2.3, 5.5, 17.4 Hz, 1H), 2.91-2.75 (m, 4H), LCMS: m/z [M+H]⁺=188.1

### Synthetic scheme for preparation of 3-chloro-N-methyl-4,5,6,7-tetrahydrobenzothiophen-5-amine hydrochloride enantiomer 1 (131) and enantiomer 2 (132)

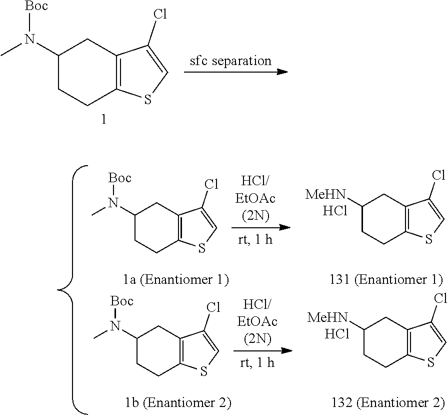

### Procedure for preparation tert-butyl N-(3-chloro-4,5,6,7-tetrahydrobenzothiophen-5-yl)-N-methyl-carbamate enantiomer 1 (1a) and enantiomer 2 (1b)

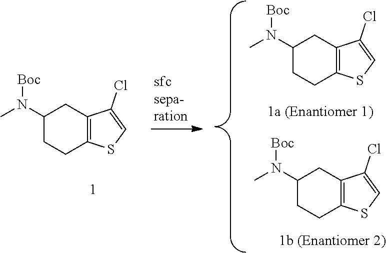 [SMILES: CN(C(=O)OC(C)(C)C)C1CCc2scc(Cl)c2C1 ; CN(C(=O)OC(C)(C)C)C1CCc2scc(Cl)c2C1 ; CN(C(=O)OC(C)(C)C)C1CCc2scc(Cl)c2C1]

Racemic tert-butyl N-(3-chloro-4,5,6,7-tetrahydrobenzothiophen-5-yl)-N-methyl-carbamate (1) was separated by SFC (column: DAICEL CHIRALPAK IG (250 mm*50 mm, 10 μm); mobile phase: [CO₂-EtOH (0.1% NH₃H₂O)]; B %:10%, isocratic elution mode) to afford the 1^{st} enantiomer (1a) (RT=0.918 min, 100 mg, 347.46 μmol) and the 2^{nd} enantiomer (1b) (RT=1.054 min, 110 mg, 382.20 μmol), both as colorless oils.

### Procedure for preparation of 3-chloro-N-methyl-4,5,6,7-tetrahydrobenzothiophen-5-amine hydrochloride enantiomer 1 (131)

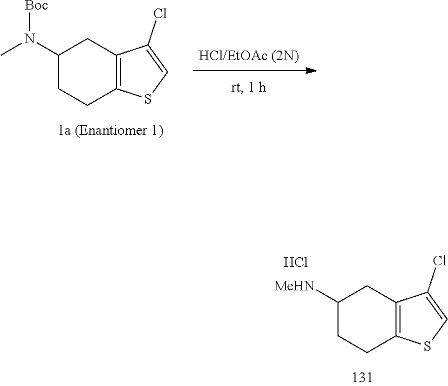 [SMILES: CN(C(=O)OC(C)(C)C)C1CCc2scc(Cl)c2C1 ; Cl ; CNC1CCc2scc(Cl)c2C1]

To a solution of tert-butyl N-(3-chloro-4,5,6,7-tetrahydrobenzothiophen-5-yl)-N-methyl-carbamate enantiomer 1 (1a) (100 mg, 347.46 μmol, 1 eq) in EtOAc (2 mL) was added HCl/EtOAc (4 M, 2 mL, 23.02 eq). The mixture was stirred at 25° C. for 1 hr. The reaction mixture was then filtered and the cake was washed with EtOAc (10 mL) to afford 3-chloro-N-methyl-4,5,6,7-tetrahydrobenzothiophen-5-amine hydrochloride enantiomer 1 (131) (59.27 mg, 280.08 μmol, 84.54% yield) as a white solid. ¹H NMR (400 MHz, Methanol-d4) δ=7.20 (s, 1H), 3.60-3.51 (m, 1H), 3.11 (dd, J=5.3, 16.0 Hz, 1H), 3.03-2.90 (m, 2H), 2.82 (s, 3H), 2.64-2.52 (m, 1H), 2.39-2.30 (m, 1H), 2.02-1.89 (m, 1H). LCMS: m/z [M+H]⁺=201.8.

### Procedure for preparation of 3-chloro-N-methyl-4,5,6,7-tetrahydrobenzothiophen-5-amine hydrochloride enantiomer2 (132)

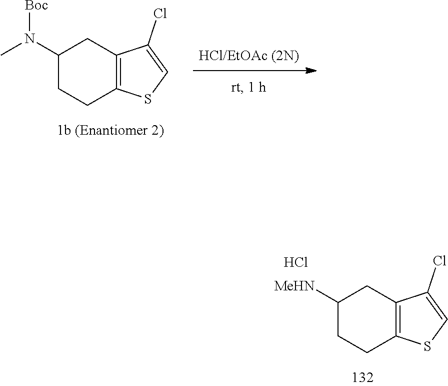 [SMILES: CN(C(=O)OC(C)(C)C)C1CCc2scc(Cl)c2C1 ; Cl ; CNC1CCc2scc(Cl)c2C1]

To a solution of tert-butyl N-(3-chloro-4,5,6,7-tetrahydrobenzothiophen-5-yl)-N-methyl-carbamate enantiomer 2 (1b) (110 mg, 364.44 μmol, 1 eq) in EtOAc (2 mL) was added HCl/EtOAc (4 M, 2 mL, 23.02 eq). The mixture was stirred at 25° C. for 1 hr. The reaction mixture was then filtered and the cake was washed with EtOAc (10 mL) to afford 3-chloro-N-methyl-4,5,6,7-tetrahydrobenzothiophen-5-amine hydrochloride enantiomer 2 (132) (56.37 mg, 233.22 μmol, 63.99% yield, 98.5) as a white solid. ¹H NMR (400 MHz, METHANOL-d₄) δ=7.20 (s, 1H), 3.55 (dddd, J=2.8, 5.6, 8.6, 11.3 Hz, 1H), 3.16-3.07 (m, 1H), 3.03-2.91 (m, 2H), 2.82 (s, 3H), 2.62-2.52 (m, 1H), 2.39-2.29 (m, 1H), 2.01-1.89 (m, 1H). LCMS: m/z [M+H]⁺=201.8.

### Synthetic scheme for preparation of 6-(methylamino)-4,5,6,7-tetrahydro-2-benzothiophene-1-carbonitrile hydrochloride enantiomer 1 (133) and enantiomer 2 (134)

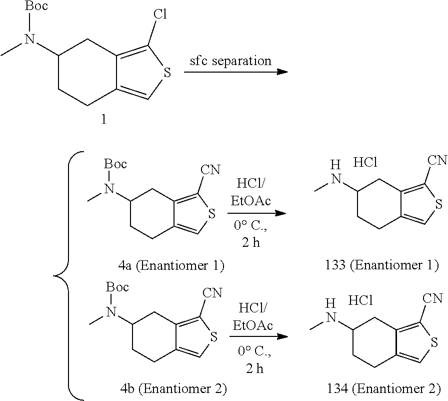

### Procedure for preparation of tert-butyl N-(3-cyano-4,5,6,7-tetrahydro-2-benzothiophen-5-yl)-N-methyl-carbamate enantiomer 1 (4a) and enantiomer 2 (4b)

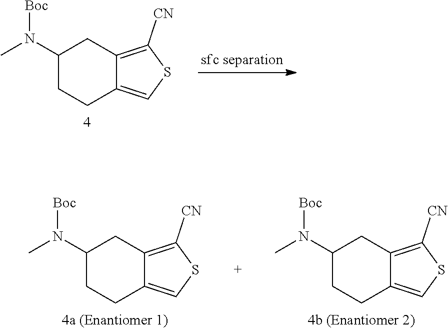

Racemic tert-butyl N-(3-cyano-4,5,6,7-tetrahydro-2-benzothiophen-5-yl)-N-methyl-carbamate (4) was separated by SFC (column: DAICEL CHIRALPAK IG (250 mm*30 mm, 10 μm); mobile phase: [CO₂-MeOH (0.1% NH₃H₂O)]; B %:15%, isocratic elution mode) to give tert-butyl N-(3-cyano-4,5,6,7-tetrahydro-2-benzothiophen-5-yl)-N-methyl-carbamate enantiomer 1 (4a) (1st peak in SFC, Rt=1.085 min) and enantiomer 2 (4b) (2nd peak in SFC, Rt=1.391 min).

### Procedure for preparation of 6-(methylamino)-4,5,6,7-tetrahydro-2-benzothiophene-1-carbonitrile hydrochloride enantiomer 1 (133)

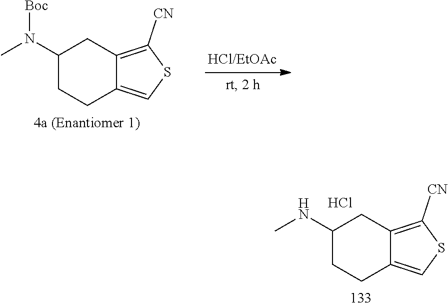

To a solution of tert-butyl N-(3-cyano-4,5,6,7-tetrahydro-2-benzothiophen-5-yl)-N-methyl-carbamate enantiomer 1 (4a) (74 mg, 253.08 μmol, 1 eq) in EtOAc (1 mL) was added HCl/EtOAc (4 M, 63.27 μL, 1 eq). The mixture was stirred at 25° C. for 2 hrs. The reaction mixture was concentrated under reduced pressure to remove EtOAc. The residue was purified by prep-HPLC (HCl condition: Phenomenex luna C18 150*25 mm*10 μm; mobile phase: [water(HCl)—CH₃CN]; gradient:1%-20% B over 10 min). Compound 6-(methylamino)-4,5,6,7-tetrahydro-2-benzothiophene-1-carbonitrile hydrochloride enantiomer 1 (133) (16.67 mg, 72.88 μmol, 28.80% yield) was obtained as a white solid. ¹H NMR (400 MHz, Methanol-d₄) δ 7.53 (s, 1H), 3.62-3.52 (m, 1H), 3.50-3.41 (m, 1H), 3.02 (td, J=4.6, 16.9 Hz, 1H), 2.94-2.75 (m, 5H), 2.36-2.27 (m, 1H), 1.89 (dtd, J=5.4, 11.1, 12.9 Hz, 1H). LCMS: m/z [M+H]⁺=193.2.

### Procedure for preparation of 6-(methylamino)-4,5,6,7-tetrahydro-2-benzothiophene-1-carbonitrile hydrochloride enantiomer 2 (134)

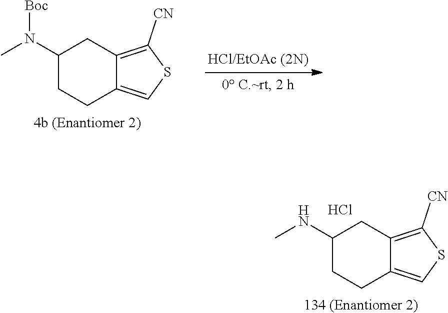

To a solution of tert-butyl N-(3-cyano-4,5,6,7-tetrahydro-2-benzothiophen-5-yl)-N-methyl-carbamate enantiomer 2 (4b) (65 mg, 222.30 μmol, 1 eq) in EtOAc (1 mL) was added HCl/EtOAc (4 M, 2.32 mL, 41.77 eq). The mixture was stirred at 25° C. for 2 hrs. The reaction mixture was concentrated under reduced pressure to remove EtOAc. The crude product was triturated with EtOAc at 25° C. for 30 min. Compound 6-(methylamino)-4,5,6,7-tetrahydro-2-benzothiophene-1-carbonitrile hydrochloride enantiomer 2 (134) (49.69 mg, 217.23 μmol, 97.72% yield) was obtained as a white solid. ¹H NMR (400 MHz, Methanol-d₄) δ 7.53 (s, 1H), 3.61-3.52 (m, 1H), 3.50-3.42 (m, 1H), 3.02 (td, J=4.8, 17.0 Hz, 1H), 2.92-2.77 (m, 5H), 2.37-2.26 (m, 1H), 1.88 (dtd, J=5.4, 11.0, 12.9 Hz, 1H). LCMS: m/z [M+H]⁺=193.1

### Synthetic scheme for preparation of 6,7-dihydro-5H-thieno[3,2-b]pyran-6-amine hydrochloride enantiomer 1 (135) and enantiomer 2 (136)

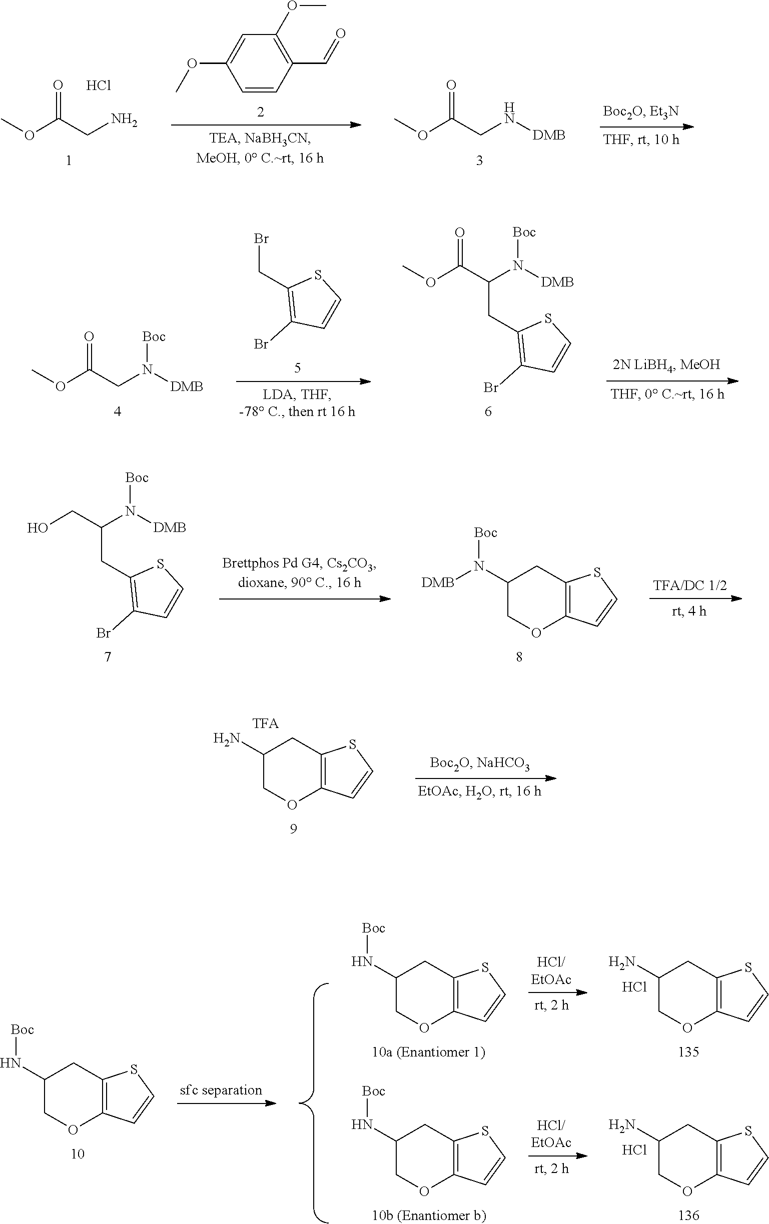

### Procedure for preparation of methyl (2,4-dimethoxybenzyl)glycinate (3)

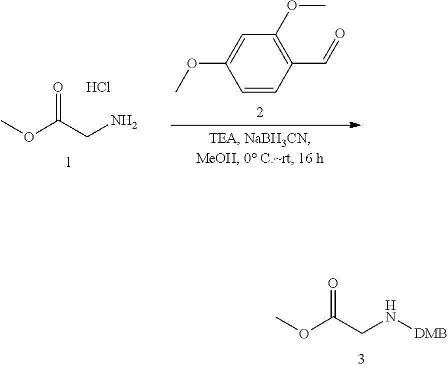

To a solution of methyl glycinate hydrochloride (10.08 g, 72.21 mmol, 1.2 eq) in MeOH (150 mL) was added TEA (6.09 g, 60.18 mmol, 8.38 mL, 1 eq) and AcOH (361.38 mg, 6.02 mmol, 344.50 μL, 0.1 eq), then was added 2,4-dimethoxybenzaldehyde (10 g, 60.18 mmol, 1 eq) and NaBH₃CN (5.67 g, 90.27 mmol, 1.5 eq) at 0° C. The mixture was stirred at 20° C. for 16 hr. The reaction mixture was quenched by addition H₂O 10 mL, concentrated under reduced pressure to give a residue. The residue was purified by flash silica gel chromatography (ISCO®; 80 g SepaFlash® Silica Flash Column, eluent 0~30% B (Ethyl acetate/NH₃·MeOH=50/1)/Petroleum ether) to afford methyl (2,4-dimethoxybenzyl)glycinate (3) (13.4 g, 52.90 mmol, 87.91% yield) as a colorless oil. ¹H NMR (400 MHz, CHLOROFORM-d) δ=7.12 (d, J=8.0 Hz, 1H), 6.48-6.41 (m, 2H), 3.81 (d, J=6.4 Hz, 6H), 3.75 (s, 2H), 3.70 (s, 3H), 3.39 (s, 2H)

### Procedure for preparation of methyl 2-[tert-butoxycarbonyl-[(2,4-dimethoxyphenyl)methyl]amino]acetate (4)

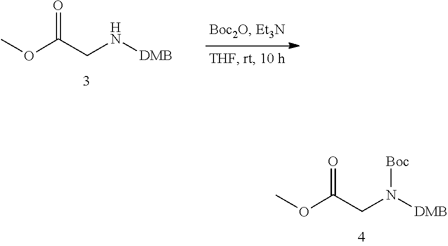

To a solution of methyl (2,4-dimethoxybenzyl)glycinate (3) (9 g, 37.61 mmol, 1 eq) and Boc₂O (9.85 g, 45.14 mmol, 10.37 mL, 1.2 eq) in THF (100 mL) was added TEA (5.71 g, 56.42 mmol, 7.85 mL, 1.5 eq). The mixture was stirred at 20° C. for 10 hr. The reaction mixture was concentrated under reduced pressure to give a residue. The residue was purified by flash silica gel chromatography (ISCO®; 80 g SepaFlash® Silica Flash Column, eluent 0~15% Ethyl acetate/Petroleum ether) to afford methyl 2-[tert-butoxycarbonyl-[(2,4-dimethoxyphenyl)methyl]amino]acetate (4) (9.3 g, 27.40 mmol, 72.85% yield) as a colorless oil. ¹H NMR (400 MHz, CHLOROFORM-d) δ=7.25-7.09 (m, 1H), 6.49-6.41 (m, 2H), 4.44 (br d, J=18.8 Hz, 2H), 4.03-3.86 (m, 2H), 3.79 (dd, J=2.8, 9.4 Hz, 6H), 3.70 (d, J=2.4 Hz, 3H), 1.46 (d, J=18.9 Hz, 9H)

### Procedure for preparation of methyl 3-(3-bromo-2-thienyl)-2-[tert-butoxycarbonyl-[(2,4-dimethoxyphenyl)methyl]amino]propanoate (6)

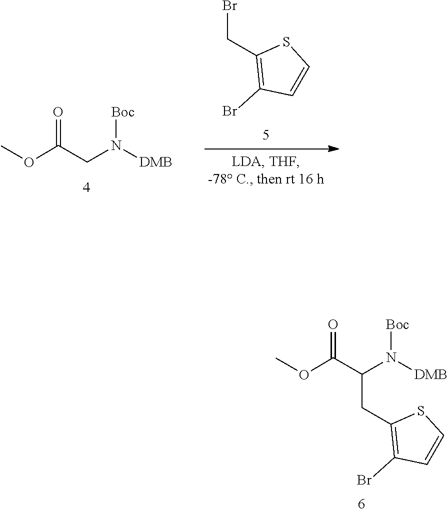

To a solution of LDA (2 M, 11.34 mL, 1.1 eq) in THF (50 mL) was added methyl 2-[tert-butoxycarbonyl-[(2,4-dimethoxyphenyl)methyl]amino]acetate (4) (7 g, 20.63 mmol, 1 eq) in THF (50 mL) at −78° C. under N₂ over 0.5 hr. After addition, the mixture was stirred at this temperature for 0.5 hr, then 3-bromo-2-(bromomethyl)thiophene (6.34 g, 24.75 mmol, 1.2 eq) in THF (50 mL) was added at −78° C. The mixture was allowed to slowly warm up to rt and stirred at −25° C. for 16 hr under N₂. The reaction mixture was quenched by addition aq. sat. NH₄Cl (100 mL), and extracted with EtOAc (80 mL*3). The combined organic layers were washed with aq. sat. NaCl (50 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~100% Ethyl acetate/Petroleum ether) to afford methyl 3-(3-bromo-2-thienyl)-2-[tert-butoxycarbonyl-[(2,4-dimethoxyphenyl)methyl]amino]propanoate (6) (7.5 g, 14.58 mmol, 70.69% yield) as yellow oil. ¹H NMR (400 MHz, CHLOROFORM-d) δ=7.22-7.06 (m, 2H), 6.86 (d, J=5.3 Hz, 1H), 6.40-6.32 (m, 2H), 4.50-4.30 (m, 1H), 4.26-4.16 (m, 1H), 4.02-3.92 (m, 1H), 3.84-3.77 (m, 4H), 3.73 (s, 3H), 3.63 (s, 3H), 3.51 (br s, 1H), 1.49 (s, 9H)

### Procedure for preparation of tert-butyl (1-(3-bromothiophen-2-yl)-3-hydroxypropan-2-yl)(2,4-dimethoxybenzyl)carbamate (7)

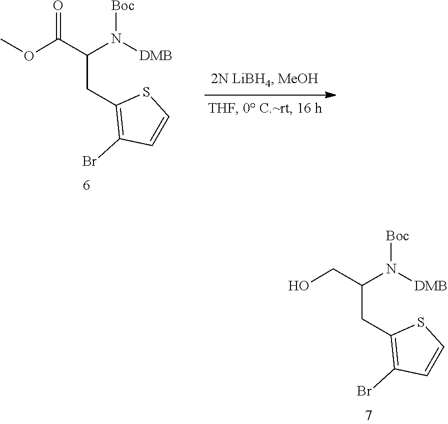

To a solution of methyl 3-(3-bromo-2-thienyl)-2-[tert-butoxycarbonyl-[(2,4-dimethoxyphenyl)methyl]amino]propanoate (6) (7.5 g, 14.58 mmol, 1 eq) in THF (80 mL) was added LiBH₄ (2 M, 29.16 mL, 4 eq) at 0° C., then MeOH (1 mL) was added. The mixture was stirred at 25° C. for 16 hr. The reaction mixture was quenched by addition aq. sat. NH₄Cl (100 mL) at 0° C., and extracted with EtOAc (100 mL*2). The combined organic layers were washed with aq. sat. NaCl (100 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~30% Ethyl acetate/Petroleum ether) to afford tert-butyl (1-(3-bromothiophen-2-yl)-3-hydroxypropan-2-yl)-(2,4-dimethoxybenzyl)carbamate (7) (6.2 g, 12.39 mmol, 84.98% yield) as yellow oil. ¹H NMR (400 MHz, CHLOROFORM-d) δ=7.16 (d, J=5.3 Hz, 1H), 7.13-7.02 (m, 1H), 6.91 (d, J=5.4 Hz, 1H), 6.42-6.41 (m, 1H), 6.42 (br s, 1H), 4.45 (br d, J=15.4 Hz, 1H), 3.80 (s, 6H), 3.70 (br s, 2H), 3.50-3.29 (m, 1H), 3.23-3.02 (m, 1H), 1.50 (s, 9H)

### Procedure for preparation of tert-butyl N-(6,7-dihydro-5H-thieno[3,2-b]pyran-6-yl)-N-[(2,4-dimethoxyphenyl)methyl]carbamate (8)

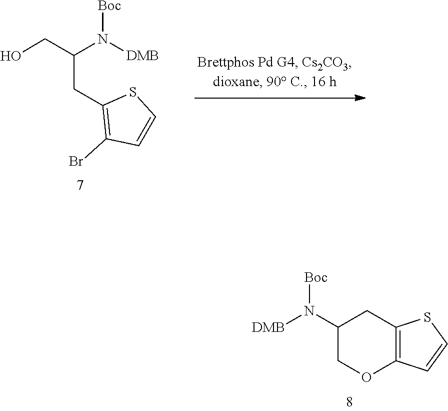

To a solution of tert-butyl (1-(3-bromothiophen-2-yl)-3-hydroxypropan-2-yl)(2,4-dimethoxybenzyl)carbamate (7) (5.2 g, 10.39 mmol, 1 eq) in dioxane (600 mL) was added BrettPhos Pd G4 (956.58 mg, 1.04 mmol, 0.1 eq) and Cs₂CO₃ (6.77 g, 20.78 mmol, 2 eq). The mixture was stirred at 90° C. for 16 hrs under N₂. The reaction mixture was filtered and concentrated under reduced pressure to give a residue. The residue was purified by flash silica gel chromatography (ISCO®; 80 g SepaFlash® Silica Flash Column, eluent 0~20% Ethyl acetate/Petroleum ether) to afford tert-butyl N-(6,7-dihydro-5H-thieno[3,2-b]pyran-6-yl)-N-[(2,4-dimethoxyphenyl)methyl]carbamate (8) (3.4 g, 8.38 mmol, 80.69% yield) as yellow oil. ¹H NMR (400 MHz, CHLOROFORM-d) δ=7.20-7.07 (m, 1H), 6.95 (br d, J=5.4 Hz, 1H), 6.55 (d, J=5.3 Hz, 1H), 6.51-6.41 (m, 2H), 4.58-4.20 (m, 3H), 4.19-4.01 (m, 2H), 3.80 (d, J=12.5 Hz, 6H), 3.08 (br dd, J=10.5, 15.2 Hz, 1H), 2.83-2.66 (m, 1H), 1.48 (br s, 9H)

### Procedure for preparation of 6,7-dihydro-5H-thieno [3,2-b]pyran-6-amine TFA salt (9)

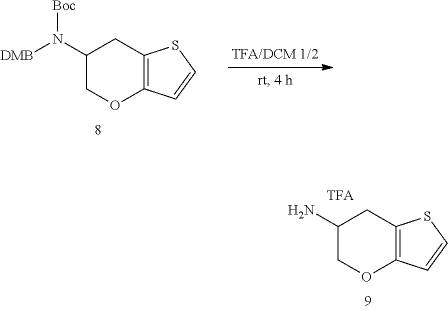

To a solution of tert-butyl N-(6,7-dihydro-5H-thieno[3,2-b]pyran-6-yl)-N-[(2,4-dimethoxyphenyl)methyl]carbamate (8) (2.9 g, 7.15 mmol, 1 eq) in DCM (20 mL) was added TFA (15.35 g, 134.62 mmol, 10 mL, 18.82 eq). The mixture was stirred at 25° C. for 4 hr. The reaction mixture was concentrated under reduced pressure to remove DCM/TFA. The crude product 6,7-dihydro-5H-thieno[3,2-b]pyran-6-amine TFA salt (9) (1.5 g, crude) was used into the next step without further purification. LCMS: m/z [M+H-NH₂]⁺=139.2.

### Procedure for preparation of tert-butyl N-(6,7-dihydro-5H-thieno[3,2-b]pyran-6-yl)carbamate (10)

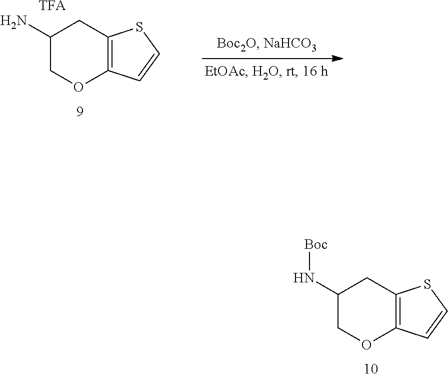 [SMILES: "TFA" ; NC1COc2ccsc2C1 ; CC(C)(C)OC(=O)NC1COc2ccsc2C1]

To a solution of 6,7-dihydro-5H-thieno[3,2-b]pyran-6-amine TFA salt (9) (1.5 g, 9.66 mmol, 1 eq) in EtOAc (10 mL) and H₂O (5 mL) was added NaHCO₃ (4.06 g, 48.32 mmol, 1.88 mL, 5 eq) and (Boc)₂O (4.22 g, 19.33 mmol, 4.44 mL, 2 eq). The mixture was stirred at 25° C. for 16 hr. The reaction mixture was poured into H₂O (30 mL) and extracted with EtOAc (20 mL*3). The combined organic phases were washed with brine (10 mL), dried over anhydrous sodium sulfate, filtered and concentrated under vacuum to give a residue. The residue was purified by column chromatography (SiO₂, Petroleum ether/Ethyl acetate=10/1 to 5/1) to give desired compound as yellow oil, which was further purified by prep-HPLC (column: Phenomenex Luna C18 (250*70 mm, 10 μm); mobile phase: [water (NH₄HCO₃)-ACN]; gradient:45%-75% B over 20 min) to afford tert-butyl N-(6,7-dihydro-5H-thieno[3,2-b]pyran-6-yl)carbamate (10) (1.1 g, 4.31 mmol, 44.58% yield) as yellow oil. LCMS: m/z [M+H-tBu]⁺=200.0.

### Procedure for preparation of tert-butyl N-(6,7-dihydro-5H-thieno[3,2-b]pyran-6-yl)carbamate enantiomer 1 (10a) and enantiomer 2 (10b)

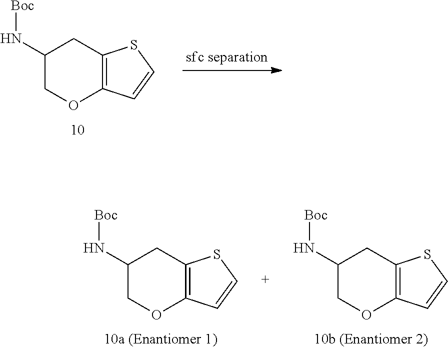

Racemic tert-butyl N-(6,7-dihydro-5H-thieno[3,2-b]pyran-6-yl)carbamate (10) (200 mg) was separated to enantiomers by SFC (column: DAICEL CHIRALPAK IC (250 mm*50 mm, 10 μm); mobile phase: [CO₂-iPrOH (0.1% NH₃H₂O)]; B %:10%, isocratic elution mode) to afford tert-butyl N-(6,7-dihydro-5H-thieno[3,2-b]pyran-6-yl)carbamate enantiomer 1 (10a) (70 mg, 274.15 μmol, 35.00% yield) and enantiomer 2 (10b) (80 mg, 313.32 μmol, 40.00% yield) both as a yellow oil.

### Procedure for preparation of 6,7-dihydro-5H-thieno[3,2-b]pyran-6-amine hydrochloride enantiomer 1 (135)

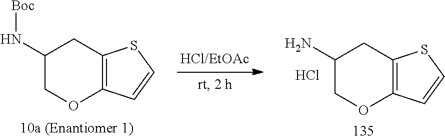

To a solution of tert-butyl N-(6,7-dihydro-5H-thieno[3,2-b]pyran-6-yl)carbamate enantiomer 1 (10a) (70 mg, 274.15 μmol, 1 eq) in EtOAc (1 mL) was added HCl/EtOAc (4 M, 1 mL, 14.59 eq). The mixture was stirred at 25° C. for 2 hr. The mixture was concentrated and triturated with EtOAc at 25° C. for 10 min to afford 6,7-dihydro-5H-thieno[3,2-b]pyran-6-amine hydrochloride enantiomer 1 (135) (44.22 mg, 228.39 μmol, 83.31% yield) as a white solid. ¹H NMR (400 MHz, Methanol-d₄) δ=7.21 (d, J=5.4 Hz, 1H), 6.67 (d, J=5.5 Hz, 1H), 4.34-4.26 (m, 1H), 4.22-4.15 (m, 1H), 3.90 (br d, J=2.6 Hz, 1H), 3.28-3.22 (m, 1H), 2.86 (br d, J=16.9 Hz, 1H)

### Procedure for preparation of 6,7-dihydro-5H-thieno[3,2-b]pyran-6-amine hydrochloride enantiomer 2 (136)

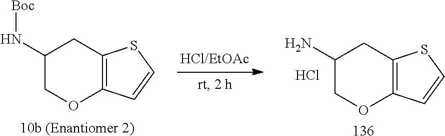

To a solution of tert-butyl N-(6,7-dihydro-5H-thieno[3,2-b]pyran-6-yl)carbamate enantiomer 2 (10b) (80 mg, 313.32 μmol, 1 eq) in EtOAc (1 mL) was added HCl/EtOAc (4 M, 1.14 mL, 14.59 eq). The mixture was stirred at 25° C. for 2 hr. The mixture was concentrated and triturated with EtOAc at 25° C. for 10 min. to afford 6,7-dihydro-5H-thieno[3,2-b]pyran-6-amine hydrochloride enantiomer 2 (136) (52.33 mg, 270.28 μmol, 86.26% yield) as a yellow solid. ¹H NMR (400 MHz, METHANOL-d₄) δ=7.21 (d, J=5.4 Hz, 1H), 6.67 (d, J=5.4 Hz, 1H), 4.36-4.26 (m, 1H), 4.23-4.14 (m, 1H), 3.95-3.88 (m, 1H), 3.30-3.26 (m, 1H), 2.87 (br d, J=17.3 Hz, 1H)

### Synthetic scheme for preparation of 3-fluoro-N-methyl-4,5,6,7-tetrahydrobenzothiophen-5-amine hydrochloride enantiomer 1 (137) and enantiomer 2 (138)

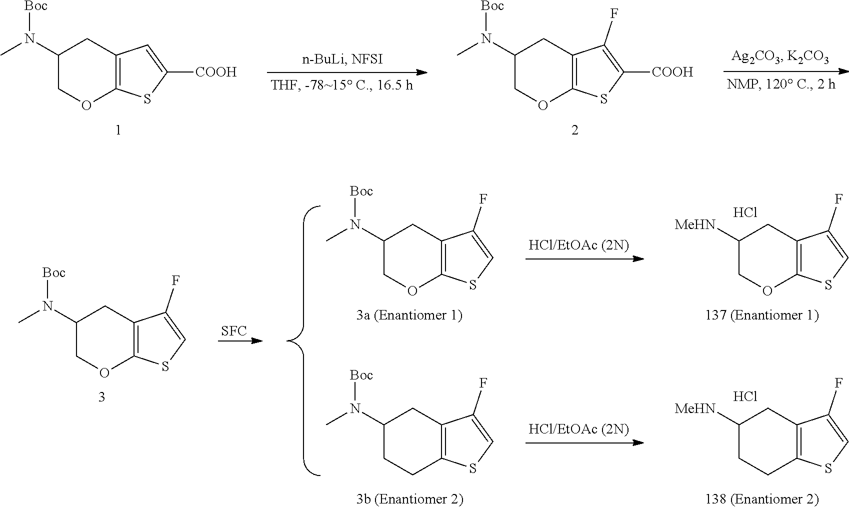

### Procedure for preparation of 5-[tert-butoxycarbonyl(methyl)amino]-3-fluoro-4,5,6,7-tetrahydrobenzothiophene-2-carboxylic acid (2)

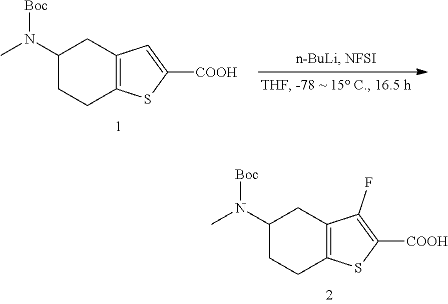 [SMILES: CN(C(=O)OC(C)(C)C)C1CCc2sc(C(=O)O)cc2C1 ; CN(C(=O)OC(C)(C)C)C1CCc2sc(C(=O)O)c(F)c2C1]

To a solution of 5-[tert-butoxycarbonyl(methyl)amino]-4,5,6,7-tetrahydrobenzothiophene-2-carboxylic acid (1) (1.9 g, 6.10 mmol, 1 eq) in THF (15 ml) was added n-BuLi (2.5 M, 5.13 mL, 2.1 eq) at −78° C. The mixture was stirred at −78° C. for 0.5 hr. Then NFSI (2.31 g, 7.32 mmol, 1.2 eq) in THF (10 mL) was added. The reaction mixture was allowed to warm up to 15° C. and stirred for 16 hr. The reaction mixture was quenched with H₂O (30 mL) and then pH adjusted to 5 with aq. citric acid. The aqueous phase was extracted with ethyl acetate (30 mL*3). The combined organic phases were dried with anhydrous Na₂SO₄, filtered and concentrated under vacuum to give a residue. The residue was purified by prep-HPLC (column: Phenomenex luna C18 150*40 mm*15 μm; mobile phase: [water(FA)-ACN]; gradient:48%-78% B over 11 min). 5-[tert-butoxycarbonyl(methyl)amino]-3-fluoro-4,5,6,7-tetrahydrobenzothiophene-2-carboxylic acid (2) (1.9 g, crude) was isolated as white solid. LCMS: m/z [M+Na]⁺=352.2

### Procedure for preparation of tert-butyl N-(3-fluoro-4,5,6,7-tetrahydrobenzothiophen-5-yl)-N-methyl-carbamate enantiomer 1 (3a) and enantiomer 2 (3b)

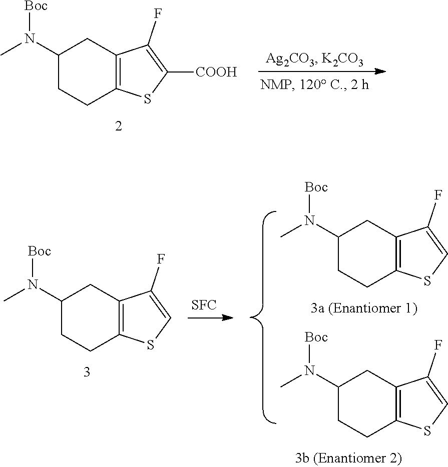

To a solution of 5-[tert-butoxycarbonyl(methyl)amino]-3-fluoro-4,5,6,7-tetrahydrobenzothiophene-2-carboxylic acid (2) (1.5 g, 4.55 mmol, 1 eq) in NMP (20 mL) was added K₂CO₃ (2.01 g, 14.57 mmol, 3.2 eq) and Ag₂CO₃ (313.93 mg, 1.14 mmol, 51.66 μL, 0.25 eq). The mixture was stirred at 120° C. for 2 hours. The reaction was quenched with water (40 mL), diluted with EtOAc (40 mL), filtered and extracted with EtOAc (40 mL). The organic layer was washed with brine (50 mL), dried over anhydrous Na₂SO₄, filtered and concentrated to get a residue. The residue was purified by prep-HPLC (column: Phenomenex luna C18 150*40 mm*15 μm; mobile phase: [water(TFA)-ACN]; gradient:69%-79% B over 15 min) to afford tert-butyl N-(3-fluoro-4,5,6,7-tetrahydrobenzothiophen-5-yl)-N-methyl-carbamate (3) (0.25 g, 876.04 μmol, 19.24% yield) as light yellow oil. This material was further separated to its enantiomers by SFC with the following method: column: DAICEL CHIRALPAK IG (250 mm*50 mm, 10 μm); mobile phase: [CO₂-MeOH (0.1% NH₃H₂O)]; B %:15%, isocratic elution mode. Tert-butyl N-(3-fluoro-4,5,6,7-tetrahydrobenzothiophen-5-yl)-N-methyl-carbamate enantiomer 1 (3a) (0.1 g, 350.41 mol) was obtained as colorless oil. Tert-butyl N-(3-fluoro-4,5,6,7-tetrahydrobenzothiophen-5-yl)-N-methyl-carbamate enantiomer 2 (3b) (0.07 g, 245.29 mol) was obtained as colorless oil. LCMS: m/z [M+H-tBu]⁺=229.8

### Procedure for preparation of 3-fluoro-N-methyl-4,5,6,7-tetrahydrobenzothiophen-5-amine hydrochloride enantiomer 1 (137)

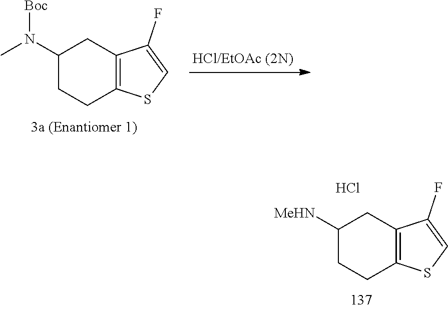 [SMILES: CN(C(=O)OC(C)(C)C)C1CCc2scc(F)c2C1 ; CNC1CCc2scc(F)c2C1 ; Cl]

To a solution of tert-butyl N-(3-fluoro-4,5,6,7-tetrahydrobenzothiophen-5-yl)-N-methyl-carbamate enantiomer 1 (3a) (0.1 g, 350.41 μmol, 1 eq) in EtOAc (2 mL) was added HCl/EtOAc (4 M, 2 mL, 22.83 eq) at 0° C. The mixture was stirred at 20° C. for 2 hours. The reaction was concentrated. Residue triturated with EtOAc to afford 3-fluoro-N-methyl-4,5,6,7-tetrahydrobenzothiophen-5-amine hydrochloride enantiomer 1 (137) (48.38 mg, 218.01 μmol, 62.22% yield) as white solid. ¹H NMR (400 MHz, METHANOL-d₄) δ=6.74 (s, 1H), 3.60-3.47 (m, 1H), 3.08 (dd, J=4.8, 15.6 Hz, 1H), 2.98-2.85 (m, 2H), 2.80 (s, 3H), 2.64-2.52 (m, 1H), 2.39-2.28 (m, 1H), 2.05-1.89 (m, 1H). LCMS: m/z [M+H]⁺=186.2

### Procedure for preparation of 3-fluoro-N-methyl-4,5,6,7-tetrahydrobenzothiophen-5-amine hydrochloride enantiomer 2 (138)

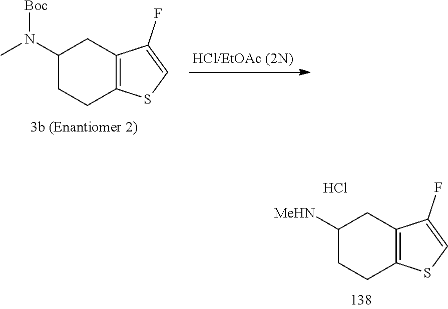 [SMILES: CN(C(=O)OC(C)(C)C)C1CCc2scc(F)c2C1 ; CNC1CCc2scc(F)c2C1 ; Cl]

To a solution of tert-butyl N-(3-fluoro-4,5,6,7-tetrahydrobenzothiophen-5-yl)-N-methyl-carbamate enantiomer 2 (3b) (0.07 g, 245.29 μmol, 1 eq) in EtOAc (2 mL) was added HCl/EtOAc (4 M, 2 mL, 32.61 eq) at 0° C. The mixture was stirred at 20° C. for 2 hours. The reaction was concentrated. To the residue was added 3 mL EA and the mixture was stirred at r.t. for 2 min, then filtered, the solid was collected and dried to afford 3-fluoro-N-methyl-4,5,6,7-tetrahydrobenzothiophen-5-amine hydrochloride enantiomer 2 (138) (50.02 mg, 225.60 μmol, 91.97% yield) as a white solid. ¹H NMR δ=6.74 (s, 1H), 3.61-3.45 (m, 1H), 3.08 (dd, J=5.2, 15.6 Hz, 1H), 2.98-2.86 (m, 2H), 2.80 (s, 3H), 2.64-2.52 (m, 1H), 2.39-2.28 (m, 1H), 2.05-1.89 (m, 1H). LCMS: m/z [M+H]⁺=186.2.

### Synthetic scheme for preparation of 3-(methylamino)-3,4-dihydro-2H-thieno[3,4-b]pyran-5-carbonitrile hydrochloride (139)

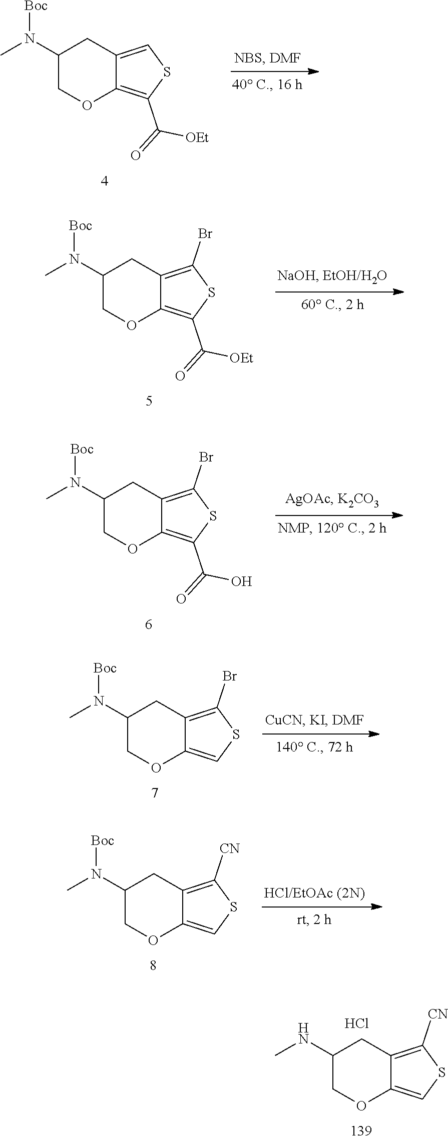

### Procedure for preparation of ethyl 5-bromo-3-[tert-butoxycarbonyl(methyl)amino]-3,4-dihydro-2H-thieno[3,4-b]pyran-7-carboxylate (5)

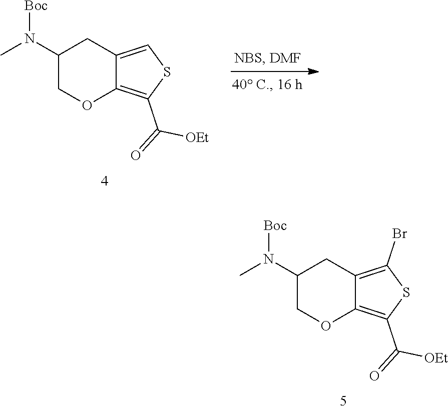 [SMILES: CCOC(=O)c1scc2c1OCC(N(C)C(=O)OC(C)(C)C)C2 ; CCOC(=O)c1sc(Br)c2c1OCC(N(C)C(=O)OC(C)(C)C)C2]

To a solution of ethyl 3-[tert-butoxycarbonyl(methyl)amino]-3,4-dihydro-2H-thieno[3,4-b]pyran-7-carboxylate (4) (790 mg, 2.31 mmol, 1 eq) in DMF (5 mL) was added NBS (494.18 mg, 2.78 mmol, 1.2 eq). The mixture was stirred at 40° C. for 16 h. The mixture was quenched with H₂O (10 mL), and then extracted with EtOAc (20 mL*2). The combined organic layers were washed with brine (10 mL), dried over anhydrous Na₂SO₄, filtered and concentrated under reduced pressure to give a residue. The residue was purified by silica gel chromatography (100-200 mesh silica gel, Petroleum ether/Ethyl acetate=110-511, Rf=0.34). Compound ethyl 5-bromo-3-[tert-butoxycarbonyl(methyl)amino]-3,4-dihydro-2H-thieno[3,4-b]pyran-7-carboxylate (5) (600 mg, 1.43 mmol, 61.69% yield) was obtained as a white solid. LCMS: m/z [M+H-tBu]⁺=366.0.

### Procedure for preparation of 5-bromo-3-[tert-butoxycarbonyl(methyl)amino]-3,4-dihydro-2H-thieno[3,4-b]pyran-7-carboxylic acid (6)

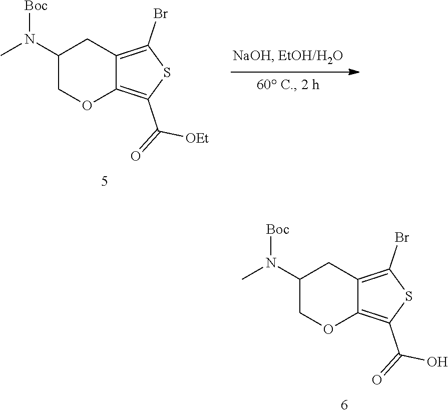 [SMILES: CCOC(=O)c1sc(Br)c2c1OCC(N(C)C(=O)OC(C)(C)C)C2 ; CN(C(=O)OC(C)(C)C)C1COc2c(C(=O)O)sc(Br)c2C1]

To a solution of ethyl 5-bromo-3-[tert-butoxycarbonyl(methyl)amino]-3,4-dihydro-2H-thieno[3,4-b]pyran-7-carboxylate (5) (450 mg, 1.07 mmol, 1 eq) in EtOH (5 mL) was added aq. NaOH (3 M, 1.07 mL, 3 eq). The mixture was stirred at 60° C. for 2 hrs. The mixture was then concentrated at reduced pressure to give a residue, water was added and mixture acidified to pH=7 with aq. HCl (1 M) and then acidified to pH=5 with aq. citric acid. The mixture was extracted with EtOAc (50 mL*3). The combined organic phases were washed with brine (10 mL*3), dried over Na₂SO₄, filtered and concentrated at reduced pressure. The crude product 5-bromo-3-[tert-butoxycarbonyl(methyl)amino]-3,4-dihydro-2H-thieno[3,4-b]pyran-7-carboxylic acid (6) (300 mg, 764.79 μmol, 71.43% yield) was obtained as a white solid and used in the next step without further purification. LCMS: m/z [M+H-tBu]⁺=337.9.

### Procedure for preparation of tert-butyl N-(5-bromo-3,4-dihydro-2H-thieno[3,4-b]pyran-3-yl)-N-methyl-carbamate (7)

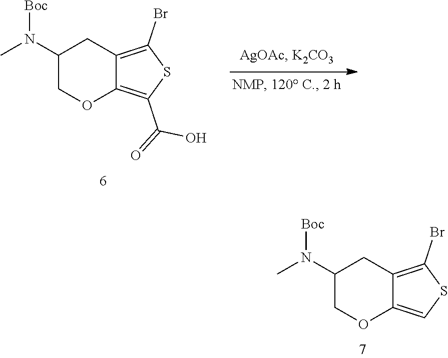 [SMILES: CN(C(=O)OC(C)(C)C)C1COc2c(C(=O)O)sc(Br)c2C1 ; CN(C(=O)OC(C)(C)C)C1COc2csc(Br)c2C1]

To a solution of 5-bromo-3-[tert-butoxycarbonyl(methyl)amino]-3,4-dihydro-2H-thieno[3,4-b]pyran-7-carboxylic acid (6) (300 mg, 764.79 μmol, 1 eq) in NMP (1 mL) were added AgOAc (12.77 mg, 76.48 μmol, 3.92 μL, 0.1 eq) and K₂CO₃ (317.10 mg, 2.29 mmol, 3 eq). The mixture was stirred at 120° C. for 2 hrs. The reaction mixture was then cooled, filtered and concentrated under reduced pressure to give a residue. The residue was purified by prep-TLC (SiO₂, Petroleum ether:Ethyl acetate=3:1, Rf=0.65). Compound tert-butyl N-(5-bromo-3,4-dihydro-2H-thieno[3,4-b]pyran-3-yl)-N-methyl-carbamate (7) (240 mg, 689.15 μmol, 90.11% yield) was obtained as a white solid. LCMS: m/z [M+H-tBu]⁺=293.9.

### Procedure for preparation of tert-butyl N-(5-cyano-3,4-dihydro-2H-thieno[3,4-b]pyran-3-yl)-N-methyl-carbamate (8)

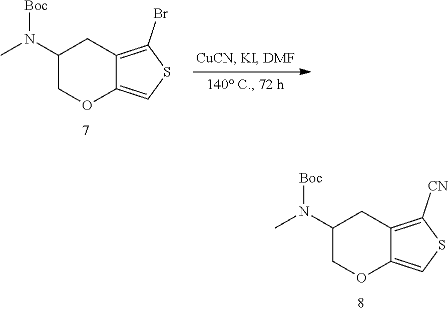 [SMILES: CN(C(=O)OC(C)(C)C)C1COc2csc(Br)c2C1 ; CN(C(=O)OC(C)(C)C)C1COc2csc(C#N)c2C1]

To a solution of tert-butyl N-(5-bromo-3,4-dihydro-2H-thieno[3,4-b]pyran-3-yl)-N-methyl-carbamate (7) (120 mg, 344.57 μmol, 1 eq) in DMF (5 mL) was added CuCN (92.58 mg, 1.03 mmol, 225.81 μL, 3 eq) and KI (5.72 mg, 34.46 μmol, 0.1 eq). The mixture was stirred at 140° C. for 72 h. The reaction mixture was filtered and concentrated under reduced pressure to give a residue. The residue was purified by prep-TLC (SiO2, Petroleum ether:Ethyl acetate=5:1, Rf=0.55). Compound tert-butyl N-(5-cyano-3,4-dihydro-2H-thieno[3,4-b]pyran-3-yl)-N-methyl-carbamate (8) (98 mg, 332.92 μmol, 96.62% yield) was obtained as a white solid. LCMS: m/z [M+H-tBu]⁺=239.0.

### Procedure for preparation of 3-(methylamino)-3,4-dihydro-2H-thieno[3,4-b]pyran-5-carbonitrile hydrochloride (139)

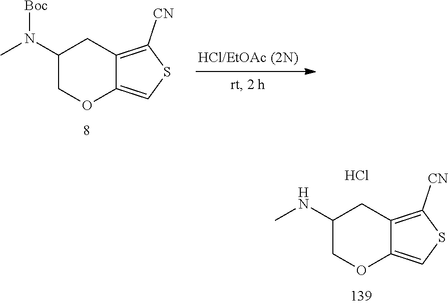 [SMILES: CN(C(=O)OC(C)(C)C)C1COc2csc(C#N)c2C1 ; Cl ; CNC1COc2csc(C#N)c2C1]

To a solution of tert-butyl N-(5-cyano-3,4-dihydro-2H-thieno[3,4-b]pyran-3-yl)-N-methyl-carbamate (8) (98 mg, 332.92 μmol, 1 eq) in EtOAc (1 mL) was added HCl/EtOAc (1 mL). The mixture was stirred at 25° C. for 2 hr. The reaction mixture was concentrated under reduced pressure to give a residue. The crude product was triturated with EtOAc (10 mL) at 25° C. for 30 min. Compound 3-(methylamino)-3,4-dihydro-2H-thieno[3,4-b]pyran-5-carbonitrile hydrochloride (139) (48.06 mg, 208.31 μmol, 62.57% yield) was obtained as a white solid. LCMS: m/z [M+H]⁺=194.1. ¹H NMR (400 MHz, Methanol-d4) δ=7.07 (s, 1H), 4.59-4.52 (m, 1H), 4.27 (dd, J=1.3, 12.6 Hz, 1H), 3.87-3.80 (m, 1H), 3.46-3.39 (m, 1H), 3.28-3.20 (m, 1H), 2.83 (s, 3H).

### Synthetic scheme for the preparation of N-methyl-3-(trifluoromethyl)-4,5,6,7-tetrahydrobenzothiophen-5-amine hydrochloride (140)

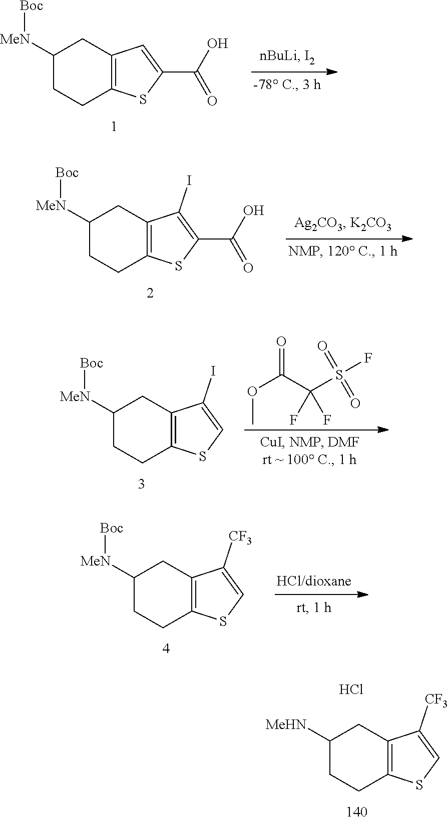

### Procedure for preparation of 5-[tert-butoxycarbonyl(methyl)amino]-3-iodo-4,5,6,7-tetrahydrobenzothiophene-2-carboxylic acid (2)

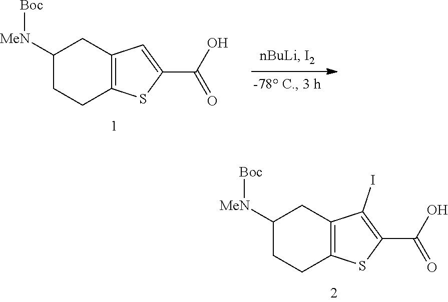 [SMILES: CN(C(=O)OC(C)(C)C)C1CCc2sc(C(=O)O)cc2C1 ; CN(C(=O)OC(C)(C)C)C1CCc2sc(C(=O)O)c(I)c2C1]

To a solution of 5-[tert-butoxycarbonyl(methyl)amino]-4,5,6,7-tetrahydrobenzothiophene-2-carboxylic acid (1) (1.7 g, 5.46 mmol, 1 eq) in THF (25 mL) was added dropwise n-BuLi (2.5 M, 5.46 mL, 2.5 eq) at −78° C. over 0.5 h. After addition, the mixture was stirred at this temperature for 0.5 h, and then I₂ (2.08 g, 8.19 mmol, 1.5 eq) in THF (15 mL) was added dropwise at −78° C. The resulting mixture was stirred at −78° C. for 2 hr. The reaction mixture was quenched by addition aq. sat. NH₄Cl 40 mL at 0° C., and then diluted with H₂O (20 mL) and extracted with EtOAc (50 mL*2). The combined organic layers were washed with aq. sat. NaCl (40 mL), dried over anhydrous Na₂SO₄, filtered and concentrated under reduced pressure to give 5-[tert-butoxycarbonyl(methyl)amino]-3-iodo-4,5,6,7-tetrahydrobenzothiophene-2-carboxylic acid (2) (0.32 g, 731.78 μmol, 13.40% yield) as a yellow solid. ¹H NMR (400 MHz, CHLOROFORM-d) δ 4.83-4.19 (m, 1H), 3.08-2.66 (m, 6H), 2.64-2.35 (m, 1H), 2.04-1.88 (m, 2H), 1.60-1.45 (m, 9H).

### Procedure for preparation of N-(3-iodo-4,5,6,7-tetrahydrobenzothiophen-5-yl)-N-methyl-carbamate (3)

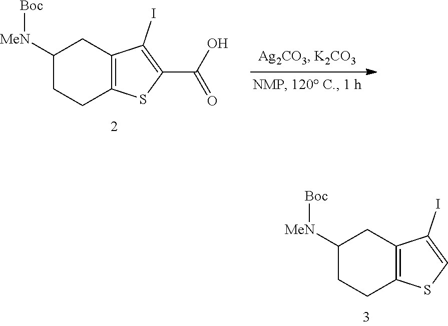 [SMILES: CN(C(=O)OC(C)(C)C)C1CCc2sc(C(=O)O)c(I)c2C1 ; CN(C(=O)OC(C)(C)C)C1CCc2scc(I)c2C1]

To a solution of 5-[tert-butoxycarbonyl(methyl)amino]-3-iodo-4,5,6,7-tetrahydrobenzothiophene-2-carboxylic acid (2) (0.27 g, 617.44 μmol, 1 eq) in NMP (5 mL) was added Ag₂CO₃ (42.56 mg, 154.36 μmol, 0.25 eq) and K₂CO₃ (256.00 mg, 1.85 mmol, 3 eq). The mixture was stirred at 120° C. for 1 hrs. The residue was diluted with EtOAc (20 mL*3) and extracted with H₂O, (10 mL*3). The combined organic layers were washed with brine (10 mL*3), dried over anhydrous sodium sulfate, filtered and concentrated under reduced pressure to give a residue. The residue was purified by flash silica gel chromatography (ISCO®; 4 g SepaFlash® Silica Flash Column, eluent 0-5% Ethyl acetate/Petroleum ether gradient @20 mL/min). Compound tert-butyl N-(3-iodo-4,5,6,7-tetrahydrobenzothiophen-5-yl)-N-methyl-carbamate (3) (0.2 g, 508.54 μmol, 82.36% yield) was obtained as a white solid. LCMS: m/z [M+H-tBu]⁺=293.9, [M+H-Boc]⁺=337.9.

### Procedure for preparation of tert-butyl N-methyl-N-[3-(trifluoromethyl)-4,5,6,7-tetrahydrobenzothiophen-5-yl]carbamate (4)

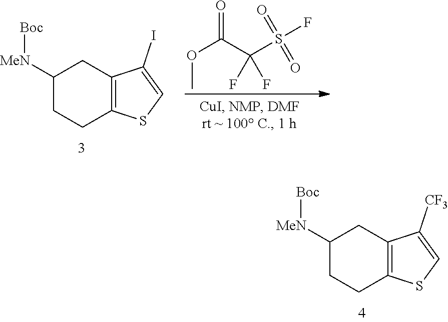

To a solution of tert-butyl N-(3-iodo-4,5,6,7-tetrahydrobenzothiophen-5-yl)-N-methyl-carbamate (3) (0.17 g, 432.26 μmol, 1 eq) in DMF (3 mL) was added CuI (411.62 mg, 2.16 mmol, 5 eq), NMP (64.28 mg, 648.39 μmol, 63.02 μL, 1.5 eq) and methyl 2,2-difluoro-2-fluorosulfonyl-acetate (415.22 mg, 2.16 mmol, 274.98 μL, 5 eq). The mixture was stirred at 100° C. for 1 hr. The reaction mixture was poured into H₂O (10 mL). The reaction was diluted with NH₃·H₂O (5 mL) and extracted with EtOAc mL (5 mL*3). The combined organic layers were washed with brine (5 mL*3), dried over anhydrous sodium sulfate, filtered and concentrated under reduced pressure to give a residue. The residue was purified by flash silica gel chromatography (ISCO®; 4 g SepaFlash® Silica Flash Column, eluent 0~10% Ethyl acetate/Petroleum ether gradient @15 mL/min) followed by prep-HPLC (column: YMC-Actus Triart C18 150*30 mm*7 μm; mobile phase: [water(FA)-ACN]; gradient:80%-100% B over 10 min). Compound tert-butyl N-methyl-N-[3-(trifluoromethyl)-4,5,6,7-tetrahydrobenzothiophen-5-yl]carbamate (4) (0.088 g, 262.39 μmol, 60.70% yield) was obtained as a yellow oil. ¹H NMR (400 MHz, CHLOROFORM-d) δ 7.51 (s, 1H), 4.74-4.18 (m, 1H), 3.01-2.78 (m, 6H), 2.76-2.66 (m, 1H), 2.00-1.89 (m, 2H), 1.49 (s, 9H).

### Procedure for preparation of N-methyl-3-(trifluoromethyl)-4,5,6,7-tetrahydrobenzothiophen-5-amine hydrochloride (140)

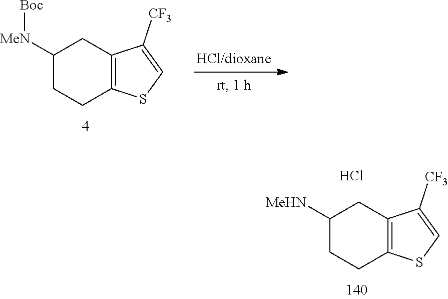 [SMILES: CN(C(=O)OC(C)(C)C)C1CCc2scc(C(F)(F)F)c2C1 ; Cl ; CNC1CCc2scc(C(F)(F)F)c2C1]

To a solution of tert-butyl N-methyl-N-[3-(trifluoromethyl)-4,5,6,7-tetrahydrobenzothiophen-5-yl]carbamate (4) (0.078 g, 232.57 μmol, 1 eq) in EtOAc (2 mL) was added HCl/EtOAc (4 M, 8.67 mL, 149.06 eq). The mixture was stirred at 25° C. for 1 hr. The reaction mixture was concentrated under vacuum to give the product. Compound N-methyl-3-(trifluoromethyl)-4,5,6,7-tetrahydrobenzothiophen-5-amine hydrochloride (140) (0.043 g, 155.60 μmol, 66.91% yield) was obtained as a white solid ¹H NMR (400 MHz, METHANOL-d4) δ 7.86 (s, 1H), 3.66-3.51 (m, 1H), 3.25 (br dd, J=5.4, 16.0 Hz, 1H), 3.10-2.89 (m, 2H), 2.86-2.67 (m, 4H), 2.43-2.32 (m, 1H), 1.98 (dddd, J=6.1, 9.8, 10.8, 12.9 Hz, 1H). LCMS: m/z [M+H]⁺=236.2.

### Synthetic scheme for preparation of N, 5-dimethyl-3-(trifluoromethyl)-6,7-dihydro-4H-2-benzothiophen-5-amine hydrochloride (141)

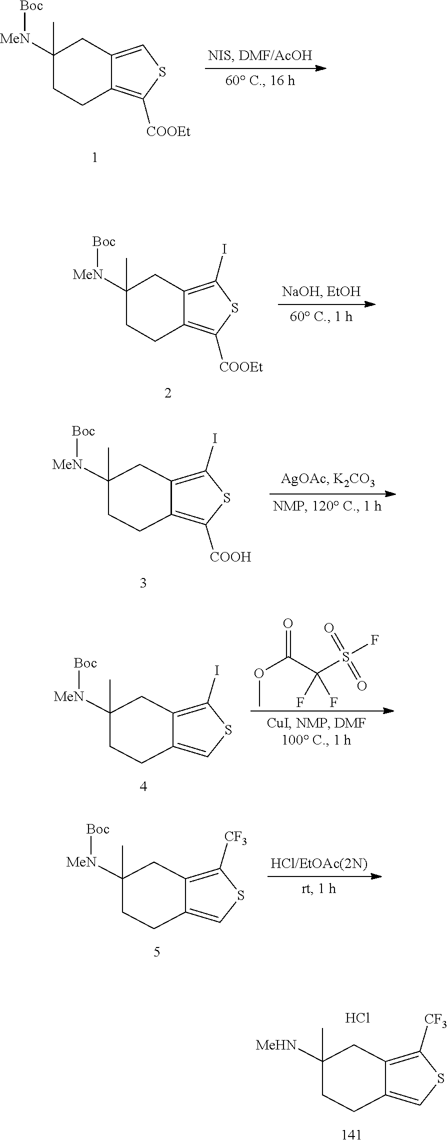

### Procedure for ethyl 5-[tert-butoxycarbonyl(methyl)amino]-3-iodo-5-methyl-6,7-dihydro-4H-2-benzothiophene-1-carboxylate (2)

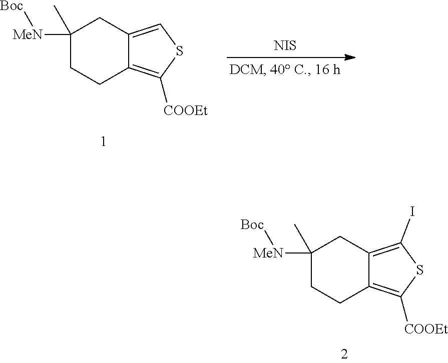 [SMILES: CCOC(=O)c1scc2c1CCC(C)(N(C)C(=O)OC(C)(C)C)C2 ; "NIS" ; CCOC(=O)c1sc(I)c2c1CCC(C)(N(C)C(=O)OC(C)(C)C)C2]

To a solution of ethyl 5-[tert-butoxycarbonyl(methyl)amino]-5-methyl-6,7-dihydro-4H-2-benzothiophene-1-carboxylate (1) (660 mg, 1.87 mmol, 1 eq) in DCM (5 mL) was added NIS (462.09 mg, 2.05 mmol, 1.1 eq). The mixture was stirred at 40° C. for 16 hr. The reaction mixture was diluted with water (10 mL) and concentrated under vacuum to remove DCM. The aqueous phase was extracted with EtOAc (10 mL*3). The combined organic phases were washed with brine (10 mL), dried over anhydrous sodium sulfate, filtered and concentrated under vacuum to give a residue. The residue was purified by flash silica gel chromatography (ISCO; 4 g SepaFlash Silica Flash Column, eluent 0~20% Ethyl acetate/Petroleum ether gradient @40 mL/min) to yield ethyl 5-[tert-butoxycarbonyl(methyl)amino]-3-iodo-5-methyl-6,7-dihydro-4H-2-benzothiophene-1-carboxylate (2) (600 mg, 1.25 mmol, 67.03% yield) as a yellow oil. ¹H NMR (400 MHz, METHANOL-d4) δ 4.29 (q, J=7.2 Hz, 2H), 3.19-3.07 (m, 2H), 2.97-2.86 (m, 1H), 2.84-2.71 (m, 1H), 2.65 (s, 3H), 2.49 (d, J=16.9 Hz, 1H), 1.74-1.61 (m, 1H), 1.50-1.41 (m, 12H), 1.34 (t, J=7.1 Hz, 3H)

### Procedure for preparation of 5-[tert-butoxycarbonyl(methyl)amino]-3-iodo-5-methyl-6,7-dihydro-4H-2-benzothiophene-1-carboxylic acid (3)

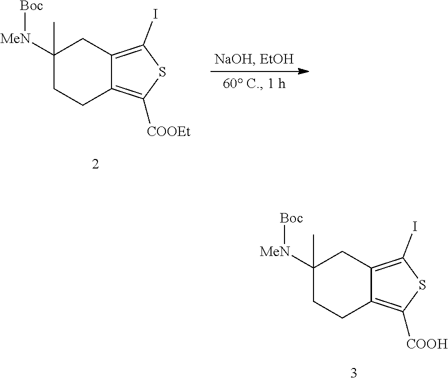

To a mixture of ethyl 5-[tert-butoxycarbonyl(methyl)amino]-3-iodo-5-methyl-6,7-dihydro-4H-2-benzothiophene-1-carboxylate (2) (500 mg, 1.04 mmol, 1 eq) in EtOH (10 mL) and H₂O (5 mL) was added NaOH (125 mg, 3.13 mmol, 3 eq). The resulting mixture was stirred at 60° C. for 1 h. The reaction mixture was adjusted pH to 5-6 with HCl (1 mol/L in water) and extracted with EtOAc (15 mL*2). The combined organic phases were washed with brine (15 mL), dried over anhydrous sodium sulfate, filtered and concentrated under vacuum to give 5-[tert-butoxycarbonyl(methyl)amino]-3-iodo-5-methyl-6,7-dihydro-4H-2-benzothiophene-1-carboxylic acid (3) (380 mg, 842 μmol, 80.7% yield) as a light yellow solid which was used into next step without further purification. ¹H NMR (400 MHz, CDCl₃-d) δ 3.23-3.12 (m, 1H), 3.08 (dd, J=1.6, 16.8 Hz, 1H), 2.97-2.78 (m, 2H), 2.68 (s, 3H), 2.44 (d, J=17.2 Hz, 1H), 1.69-1.56 (m, 1H), 1.48 (s, 9H), 1.44 (s, 3H). LCMS: m/z [M+H-Boc]⁺=352.1.

### Procedure for preparation of tert-butyl N-(3-iodo-5-methyl-6,7-dihydro-4H-2-benzothiophen-5-yl)-N-methyl-carbamate (4)

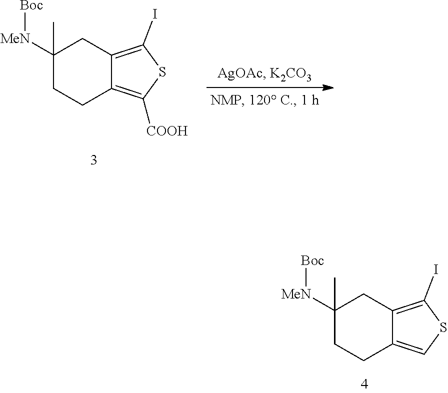

To a solution of 5-[tert-butoxycarbonyl(methyl)amino]-3-iodo-5-methyl-6,7-dihydro-4H-2-benzothiophene-1-carboxylic acid (3) (380 mg, 842 μmol, 1 eq) in NMP (5 mL) was added AgOAc (14 mg, 84.2 μmol, 0.1 eq) and K₂CO₃ (349 mg, 2.53 mmol, 3 eq). The resulting mixture was stirred at 120° C. for 1 h. The reaction mixture was diluted with water (50 mL) and extracted with EtOAc (10 mL*3). The combined organic phases were washed with brine (10 mL), dried over anhydrous sodium sulfate, filtered and concentrated under vacuum to give a residue. The residue was purified by flash silica gel chromatography (ISCO®; 8 g SepaFlash® Silica Flash Column, eluent 0~6% Ethyl acetate/Petroleum ether gradient @25 mL/min) to give tert-butyl N-(3-iodo-5-methyl-6,7-dihydro-4H-2-benzothiophen-5-yl)-N-methyl-carbamate (4) (300 mg, 736 μmol, 87.5% yield) as a white solid. ¹H NMR (400 MHz, CDCl₃-d) δ 7.06 (s, 1H), 3.02 (dd, J=2.0, 16.8 Hz, 1H), 2.97-2.86 (m, 1H), 2.80-2.58 (m, 5H), 2.39 (d, J=16.8 Hz, 1H), 1.65-1.58 (m, 1H), 1.47 (s, 9H), 1.44 (s, 3H). LCMS: m/z [M+H-Boc]⁺=308.1.

### Procedure for preparation of tert-butyl N-methyl-N-[5-methyl-3-(trifluoromethyl)-6,7-dihydro-4H-2-benzothiophen-5-yl]carbamate (5)

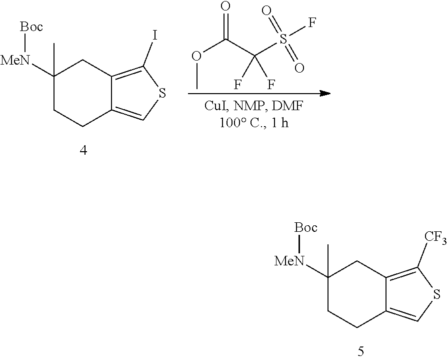

To a solution of tert-butyl N-(3-iodo-5-methyl-6,7-dihydro-4H-2-benzothiophen-5-yl)-N-methyl-carbamate (4) (300 mg, 736 μmol, 1 eq) in DMF (3 mL) was added CuI (154 mg, 810 μmol, 1.1 eq) and NMP (365 mg, 3.68 mmol, 5 eq) at 25° C. After addition, the mixture was stirred at this temperature for 10 min, then methyl 2,2-difluoro-2-fluorosulfonyl-acetate (707 mg, 3.68 mmol, 5 eq) was added dropwise at 25° C. The resulting mixture was stirred at 100° C. for 1 h. The reaction mixture coolded and was poured into water (40 mL) and extracted with EtOAc (20 mL*3). The combined organic phases were washed with brine (20 mL), dried over anhydrous sodium sulfate, filtered and concentrated under vacuum to give a residue. The residue was purified by flash silica gel chromatography (ISCO®; 4 g SepaFlash® Silica Flash Column, eluent 0~2% Ethyl acetate/Petroleum ether gradient @18 mL/min) to give tert-butyl N-methyl-N-[5-methyl-3-(trifluoromethyl)-6,7-dihydro-4H-2-benzothiophen-5-yl]carbamate (5) (250 mg, 715 μmol, 97.1% yield) as a white solid. ¹H NMR (400 MHz, CDCl₃-d) δ 7.02 (s, 1H), 3.42 (d, J=17.6 Hz, 1H), 3.06-2.94 (m, 1H), 2.80-2.59 (m, 6H), 1.68-1.58 (m, 1H), 1.47 (s, 9H), 1.44 (s, 3H). LCMS: m/z [M+H-Boc]⁺=250.2.

### Procedure for preparation of N, 5-dimethyl-3-(trifluoromethyl)-6,7-dihydro-4H-2-benzothiophen-5-amine hydrochloride (141)

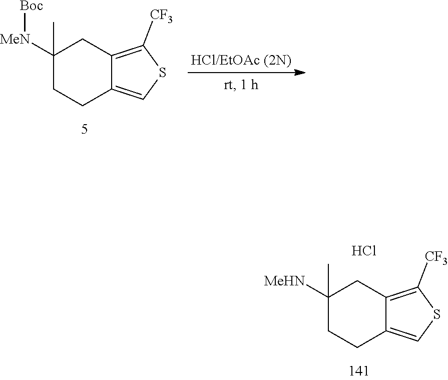 [SMILES: CN(C(=O)OC(C)(C)C)C1(C)CCc2csc(C(F)(F)F)c2C1 ; Cl ; CNC1(C)CCc2csc(C(F)(F)F)c2C1]

To the mixture of tert-butyl N-methyl-N-[5-methyl-3-(trifluoromethyl)-6,7-dihydro-4H-2-benzothiophen-5-yl]carbamate (5) (80 mg, 228.96 μmol, 1 eq) in EtOAc (2 mL) was added HCl/EtOAc (4 M, 2 mL). The resulting mixture was stirred at room temperature for 1 h. Then the reaction mixture was concentrated under vacuum to give a residue. The residue was purified by prep-HPLC (column: Phenomenex luna C18 150*25 mm*10 μm; mobile phase: [water (HCl)-ACN]; gradient: 5%-35% B over 10 min) to give N, 5-dimethyl-3-(trifluoromethyl)-6,7-dihydro-4H-2-benzothiophen-5-amine hydrochloride (141) (37.39 mg, 124.51 μmol, 54.4% yield) as a white solid. ¹H NMR (400 MHz, Methanol-d₄) δ 7.44 (s, 1H), 3.09 (q, J=16.8 Hz, 2H), 3.00-2.85 (m, 2H), 2.73 (s, 3H), 2.12-2.03 (m, 2H), 1.40 (s, 3H). LCMS: m/z [M+H]⁺=250.2.

### Synthetic scheme for preparation of 2-fluoro-N, 3-dimethyl-4,5,6,7-tetrahydrobenzothiophen-5-amine hydrochloride (142)

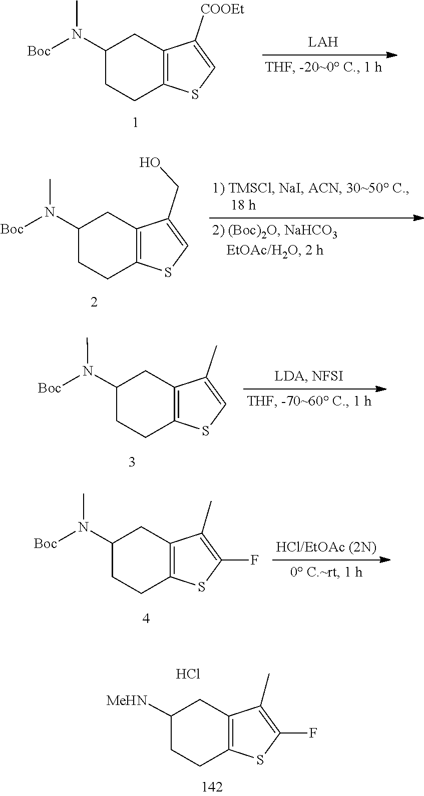 [SMILES: CCOC(=O)c1csc2c1CC(N(C)C(=O)OC(C)(C)C)CC2 ; CN(C(=O)OC(C)(C)C)C1CCc2scc(CO)c2C1 ; Cc1csc2c1CC(N(C)C(=O)OC(C)(C)C)CC2 ; Cc1c(F)sc2c1CC(N(C)C(=O)OC(C)(C)C)CC2 ; Cl ; CNC1CCc2sc(F)c(C)c2C1]

### Procedure for preparation of tert-butyl N-[3-(hydroxymethyl)-4,5,6,7-tetrahydrobenzothiophen-5-yl]-N-methyl-carbamate (2)

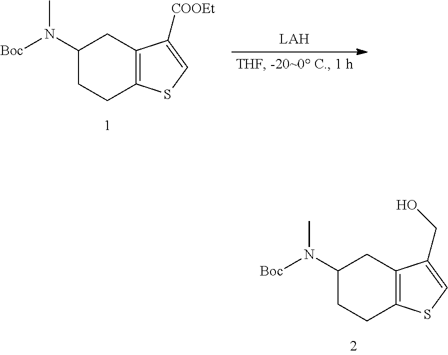 [SMILES: CCOC(=O)c1csc2c1CC(N(C)C(=O)OC(C)(C)C)CC2 ; CN(C(=O)OC(C)(C)C)C1CCc2scc(CO)c2C1]

To a solution of ethyl 5-[tert-butoxycarbonyl(methyl)amino]-4,5,6,7-tetrahydrobenzothiophene-3-carboxylate (1) (2.94 g, 8.66 mmol, 1 eq) in THF (30 mL) was added LAH (1 M in THF, 8.66 mL, 1 eq) dropwise at −20° C., the mixture was stirred at 0° C. for 1 hour. The reaction was quenched with water (0.4 mL), 10% aq. NaOH (0.4 mL) and H₂O (1.2 mL) at −10° C., then filtered. The filtrate was concentrated to afford tert-butyl N-[3-(hydroxymethyl)-4,5,6,7-tetrahydrobenzothiophen-5-yl]-N-methyl-carbamate (2) (2.2 g, crude) as white solid. LCMS: m/z [M+Na]⁺=320.1

### Procedure for preparation of tert-butyl N-methyl-N-(3-methyl-4,5,6,7-tetrahydrobenzothiophen-5-yl)carbamate (3)

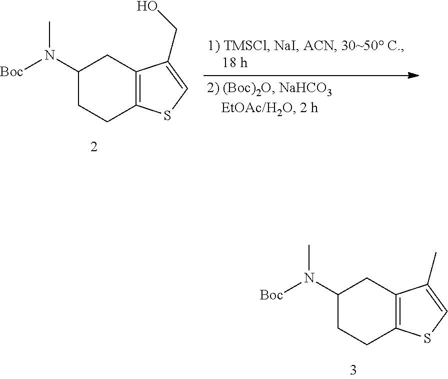 [SMILES: CN(C(=O)OC(C)(C)C)C1CCc2scc(CO)c2C1 ; Cc1csc2c1CC(N(C)C(=O)OC(C)(C)C)CC2]

To a solution of tert-butyl N-[3-(hydroxymethyl)-4,5,6,7-tetrahydrobenzothiophen-5-yl]-N-methyl-carbamate (2) (1.5 g, 5.04 mmol, 1 eq) in ACN (9 mL) was added NaI (3.02 g, 20.17 mmol, 4 eq). It was warmed to 30-35° C., then TMSCl (2.74 g, 25.22 mmol, 3.20 mL, 5 eq) was added to the mixture dropwise. The reaction was stirred at 40° C. for 16 hours. The mixture was stirred at 50° C. for another 2 hours. The reaction was cooled to rt., then quenched with 20%. aq. Na₂SO₃ (30 mL) and sat. aq. NaHCO₃ (30 mL), extracted with EtOAc (30 mL*2). The combined organic layers were separated and concentrated to get a residue. The residue was dissolved in H₂O (5 mL) and EtOAc (5 mL), (Boc)₂O (1.33 g, 6.09 mmol, 1.40 mL, 1.2 eq) and NaHCO₃ (852.59 mg, 10.15 mmol, 394.90 μL, 2 eq) was added. The mixture was stirred at 20° C. for 2 hours. The reaction was then extracted with EtOAc (50 mL*2), the organic layer was separated and washed with brine (50 mL), dried over anhydrous Na₂SO₄, filtered and concentrated to get a residue. The residue was purified by silica gel column (PE:EA=20:1 to 10:1). Compound tert-butyl N-methyl-N-(3-methyl-4,5,6,7-tetrahydrobenzothiophen-5-yl)carbamate (3) (1.4 g, crude) was obtained as off-white solid. LCMS: m/z [M+H-t-Bu]⁺=226.3. ¹H NMR (400 MHz, CHLOROFORM-d) δ=6.72 (s, 1H), 4.62-4.17 (br m, 1H), 2.91 (s, 2H), 2.82 (s, 3H), 2.67-2.43 (m, 2H), 2.10 (s, 3H), 2.00-1.84 (m, 2H), 1.49 (s, 9H)

### Procedure for preparation of tert-butyl N-(2-fluoro-3-methyl-4,5,6,7-tetrahydrobenzothiophen-5-yl)-N-methyl-carbamate (4)

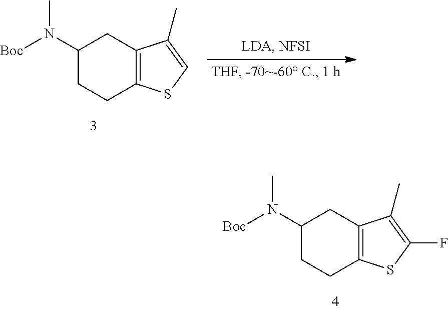 [SMILES: Cc1csc2c1CC(N(C)C(=O)OC(C)(C)C)CC2 ; Cc1c(F)sc2c1CC(N(C)C(=O)OC(C)(C)C)CC2]

To a solution of tert-butyl N-methyl-N-(3-methyl-4,5,6,7-tetrahydrobenzothiophen-5-yl)carbamate (3) (1.4 g, 4.97 mmol, 1 eq) in THF (20 mL) was added LDA (1 M, 6.47 mL, 1.3 eq) at −70° C. dropwise, the mixture was stirred at −70-−60° C. for 20 min, then NFSI (1.57 g, 4.97 mmol, 1 eq) in THF (10 mL) was added dropwise. The mixture was stirred at −70~−60° C. for 40 min. The reaction was quenched with sat. aq. NH₄Cl (30 mL) and extracted with EtOAc (20 mL*2). The organic layers were combined and washed with brine (20 mL), dried over anhydrous Na₂SO₄, filtered and concentrated to get a residue. The residue was purified by silica gel column (PE:EA=20:1 to 10:1). The crude product was further purified by prep-HPLC (column: Welch Xtimate C18 150*25 mm*5 μm; mobile phase: [water(TFA)-ACN]; gradient:65%-95% B over 9 min). Compound tert-butyl N-(2-fluoro-3-methyl-4,5,6,7-tetrahydrobenzothiophen-5-yl)-N-methyl-carbamate (4) (100 mg, 334.00 μmol, 6.71% yield) was obtained as colorless oil.

### Procedure for preparation of 2-fluoro-N, 3-dimethyl-4,5,6,7-tetrahydrobenzothiophen-5-amine hydrochloride (142)

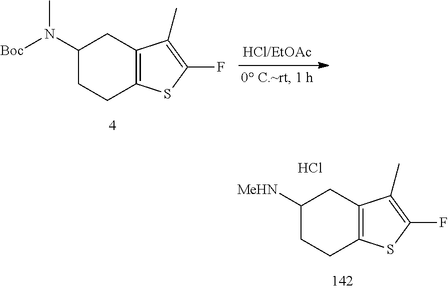 [SMILES: Cc1c(F)sc2c1CC(N(C)C(=O)OC(C)(C)C)CC2 ; CNC1CCc2sc(F)c(C)c2C1 ; Cl]

To a solution of tert-butyl N-(2-fluoro-3-methyl-4,5,6,7-tetrahydrobenzothiophen-5-yl)-N-methyl-carbamate (4) (0.085 g, 283.90 μmol, 1 eq) in EtOAc (2 mL) was added HCl/EtOAc (4 M, 4 mL, 56.36 eq) at 0° C. The mixture was stirred at 25° C. for 1 hr. The reaction mixture was concentrated under vacuum to afford 2-fluoro-N, 3-dimethyl-4,5,6,7-tetrahydrobenzothiophen-5-amine hydrochloride (142) (0.051 g, 243.96 μmol, 85.93% yield) as a white solid. ¹H NMR (400 MHz, METHANOL-d4) δ=3.55-3.45 (m, 1H), 2.97 (dd, J=5.6, 15.6 Hz, 1H), 2.82-2.81 (m, 2H), 2.80 (s, 3H), 2.48 (br dd, J=8.8, 15.6 Hz, 1H), 2.39-2.27 (m, 1H), 1.98 (d, J=2.1 Hz, 3H)), 1.86-1.97 (m, 1H). LCMS: m/z [M+H]⁺=200.2

### Synthetic scheme for preparation of 3-methyl-4,5,6,7-tetrahydrobenzothiophen-5-amine hydrochloride enantiomer 1 (143) and enantiomer 2 (144)

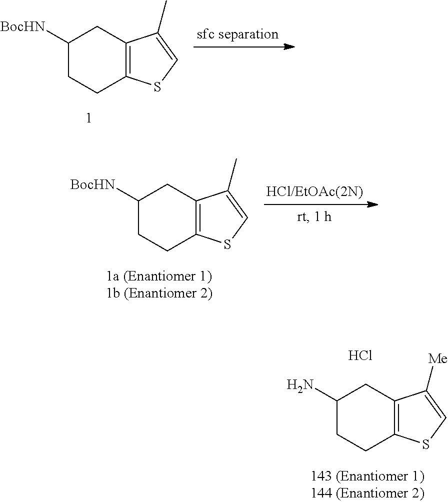 [SMILES: Cc1csc2c1CC(NC(=O)OC(C)(C)C)CC2 ; Cc1csc2c1CC(NC(=O)OC(C)(C)C)CC2 ; Cl ; Cc1csc2c1CC(N)CC2]

### Procedure for preparation of tert-butyl N-(3-methyl-4,5,6,7-tetrahydrobenzothiophen-5-yl)carbamate enantiomer 1 (1a) and enantiomer 2 (1b)

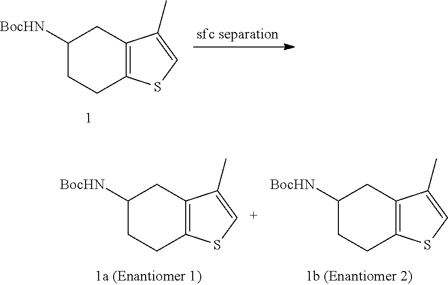 [SMILES: Cc1csc2c1CC(NC(=O)OC(C)(C)C)CC2 ; Cc1csc2c1CC(NC(=O)OC(C)(C)C)CC2 ; Cc1csc2c1CC(NC(=O)OC(C)(C)C)CC2]

Racemic tert-butyl N-(3-methyl-4,5,6,7-tetrahydrobenzothiophen-5-yl)carbamate (1) (300 mg) was separated by SFC (column: DAICEL CHIRALPAK AY (250 mm*50 mm, 10 μm); mobile phase: [CO₂-EtOH (0.1% NH₃H₂O)]; B %:12.5%, isocratic elution mode). Compound tert-butyl N-(3-methyl-4,5,6,7-tetrahydrobenzothiophen-5-yl)carbamate enantiomer 1 (1a) (130 mg, 486 μmol) was obtained as a white solid. LCMS: m/z [M+H-tBu]⁺=212.0. Compound tert-butyl N-(3-methyl-4,5,6,7-tetrahydrobenzothiophen-5-yl)carbamate enantiomer 2 (1b) (120 mg, 448 mol) was obtained as a white solid. LCMS: m/z [M+H-tBu]⁺=212.0

### Procedure for preparation of 3-methyl-4,5,6,7-tetrahydrobenzothiophen-5-amine hydrochloride enantiomer 1 (143)

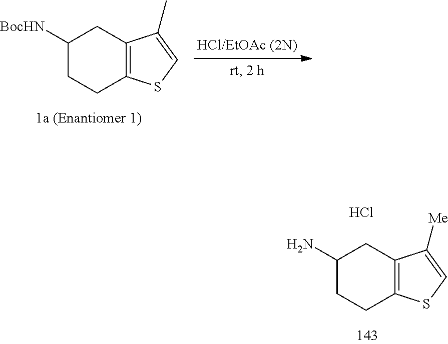 [SMILES: Cc1csc2c1CC(NC(=O)OC(C)(C)C)CC2 ; Cl ; Cc1csc2c1CC(N)CC2]

To a solution of tert-butyl N-(3-methyl-4,5,6,7-tetrahydrobenzothiophen-5-yl)carbamate enantiomer 1 (1a) (130 mg, 486.19 μmol, 1 eq) in EtOAc (2 mL) was added HCl/EtOAc (4 M, 2 mL, 16.45 eq). The mixture was stirred at 25° C. for 2 hr. The reaction mixture was filtered and the cake was washed with EtOAc (10 mL) to yield 3-methyl-4,5,6,7-tetrahydrobenzothiophen-5-amine hydrochloride enantiomer 1 (143) (92.41 mg, 451.77 μmol, 92.92% yield) as a white solid. ¹H NMR (400 MHz, METHANOL-d4) δ=6.84 (s, 1H), 3.65-3.53 (m, 1H), 3.02-2.88 (m, 3H), 2.51 (br dd, J=9.1, 15.7 Hz, 1H), 2.29-2.18 (m, 1H), 2.11 (d, J=0.9 Hz, 3H), 1.99-1.86 (m, 1H). LCMS: m/z [M+H]+=168.1

### Procedure for preparation of 3-methyl-4,5,6,7-tetrahydrobenzothiophen-5-amine hydrochloride enantiomer 2 (144)

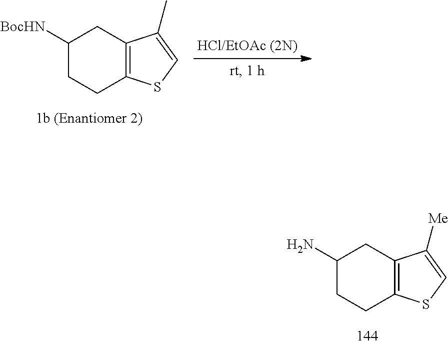 [SMILES: Cc1csc2c1CC(NC(=O)OC(C)(C)C)CC2 ; Cc1csc2c1CC(N)CC2]

To a solution of tert-butyl N-(3-methyl-4,5,6,7-tetrahydrobenzothiophen-5-yl)carbamate enanantiomer 2 (1b) (120.00 mg, 448.79 μmol, 1 eq) in EtOAc (2 mL) was added HCl/EtOAc (4 M, 2 mL, 17.83 eq). The mixture was stirred at 25° C. for 1 hr. The reaction mixture was filtered and the cake was washed with EtOAc (10 mL) to yield 3-methyl-4,5,6,7-tetrahydrobenzothiophen-5-amine hydrochloride enantiomer 2 (144) (86.6 mg, 414.57 μmol, 92.38% yield) as a white solid. ¹H NMR (400 MHz, METHANOL-d4) δ=6.84 (s, 1H), 3.65-3.54 (m, 1H), 3.01-2.88 (m, 3H), 2.56-2.45 (m, 1H), 2.28-2.17 (m, 1H), 2.11 (d, J=1.0 Hz, 3H), 1.99-1.86 (m, 1H). LCMS: m/z [M+H]⁺=168.1

### Synthetic scheme for preparation of 2-chloro-N-methyl-4,5,6,7-tetrahydrobenzothiophen-6-amine hydrochloride enantiomer 1 (145) and enantiomer 2 (146)

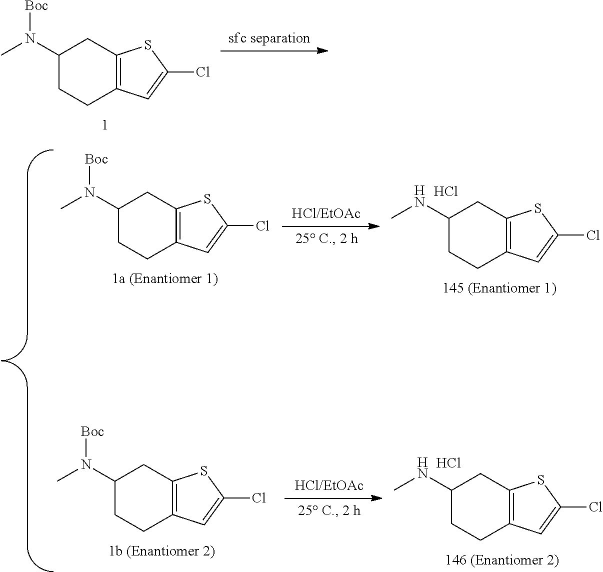

### Procedure for preparation of tert-butyl N-(2-chloro-4,5,6,7-tetrahydrobenzothiophen-6-yl)-N-methyl-carbamate enantiomer 1 (1a) and enantiomer 2 (1b)

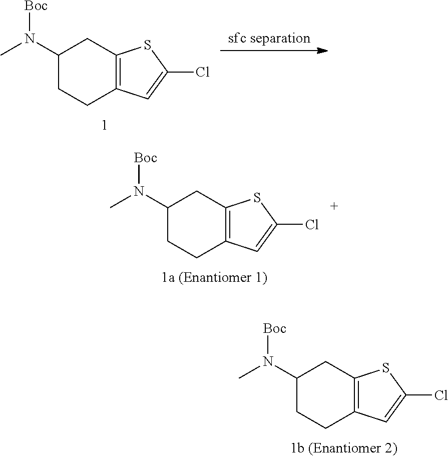 [SMILES: CN(C(=O)OC(C)(C)C)C1CCc2cc(Cl)sc2C1 ; CN(C(=O)OC(C)(C)C)C1CCc2cc(Cl)sc2C1 ; CN(C(=O)OC(C)(C)C)C1CCc2cc(Cl)sc2C1]

Racemic tert-butyl N-(2-chloro-4,5,6,7-tetrahydrobenzothiophen-6-yl)-N-methyl-carbamate (1) (200 mg) was separated by SFC (column: DAICEL CHIRALPAK IG (250 mm*50 mm, 10 μm); mobile phase: [CO₂-EtOH (0.1% NH₃H₂O)]; B %:12.5%, isocratic elution mode). Compound tert-butyl N-(2-chloro-4,5,6,7-tetrahydrobenzothiophen-6-yl)-N-methyl-carbamate enantiomer 1 (1a) (85 mg, 281.61 mol) was obtained as a white solid. LCMS: m/z [M+H-tBu]⁺=246.0. Compound tert-butyl N-(2-chloro-4,5,6,7-tetrahydrobenzothiophen-6-yl)-N-methyl-carbamate enantiomer 2 (1b) (92 mg, 304.81 mol) was obtained as a white solid. LCMS: m/z [M+H-tBu]⁺=246.0.

### Procedure for preparation of 2-chloro-N-methyl-4,5,6,7-tetrahydrobenzothiophen-6-amine hydrochloride enantiomer 1 (145)

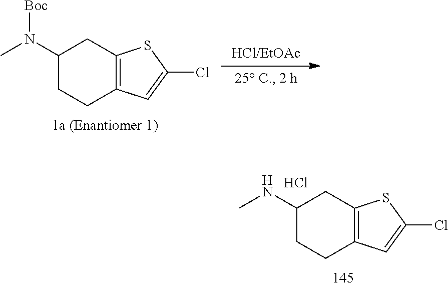 [SMILES: CN(C(=O)OC(C)(C)C)C1CCc2cc(Cl)sc2C1 ; Cl ; CNC1CCc2cc(Cl)sc2C1]

To a solution of tert-butyl N-(2-chloro-4,5,6,7-tetrahydrobenzothiophen-6-yl)-N-methyl-carbamate enantiomer 1 (1a) (85 mg, 281.61 μmol, 1 eq) in EtOAc (1 mL) was added HCl/EtOAc (4M, 1 mL). The mixture was stirred at 25° C. for 2 hr. The reaction mixture was concentrated under reduced pressure to give a residue. The crude product was triturated with EtOAc (4 mL) at 25° C. for 30 min. Compound 2-chloro-N-methyl-4,5,6,7-tetrahydrobenzothiophen-6-amine hydrochloride enantiomer 1 (145) (47.26 mg, 198.42 μmol, 70.46% yield) was obtained as a white solid. LCMS: m/z [M+H]⁺=202.2. ¹H NMR (400 MHz, METHANOL-d4) δ=6.70 (s, 1H), 3.62-3.50 (m, 1H), 3.22 (br dd, J=5.0, 16.1 Hz, 1H), 2.85-2.66 (m, 6H), 2.30-2.21 (m, 1H), 1.99-1.86 (m, 1H)

### Procedure for preparation of 2-chloro-N-methyl-4,5,6,7-tetrahydrobenzothiophen-6-amine hydrochloride enantiomer 2 (146)

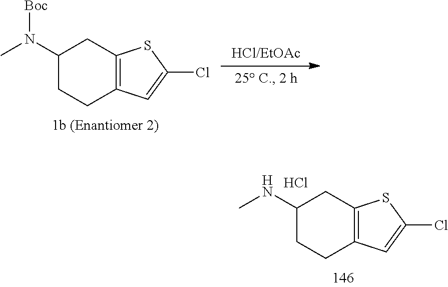 [SMILES: CN(C(=O)OC(C)(C)C)C1CCc2cc(Cl)sc2C1 ; Cl ; CNC1CCc2cc(Cl)sc2C1]

To a solution of tert-butyl N-(2-chloro-4,5,6,7-tetrahydrobenzothiophen-6-yl)-N-methyl-carbamate enantiomer 2 (1b) (92 mg, 304.81 μmol, 1 eq) in EtOAc (1 mL) was added HCl/EtOAc (4M, 1 mL). The mixture was stirred at 25° C. for 2 hrs. The reaction mixture was concentrated under reduced pressure to give a residue. The crude product was triturated with EtOAc (4 mL) at 25° C. for 30 min. Compound 2-chloro-N-methyl-4,5,6,7-tetrahydrobenzothiophen-6-amine hydrochloride enantiomer 2 (146) (53.50 mg, 224.62 μmol, 73.69% yield) was obtained as a white solid. LCMS: m/z [M+H]⁺=202.2. ¹H NMR (400 MHz, METHANOL-d₄) δ=6.70 (s, 1H), 3.61-3.50 (m, 1H), 3.22 (br dd, J=5.1, 16.0 Hz, 1H), 2.84-2.66 (m, 6H), 2.31-2.21 (m, 1H), 1.98-1.85 (m, 1H)

### Synthetic scheme for preparation of 2-chloro-N, 3-dimethyl-4,5,6,7-tetrahydrobenzothiophen-6-amine hydrochloride enantiomer 1 (147) and enantiomer 2 (148)

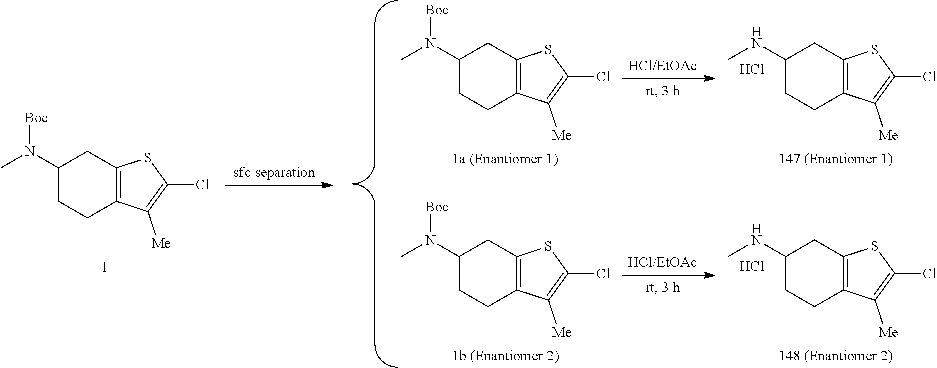

### Procedure for preparation of tert-butyl N-(2-chloro-3-methyl-4,5,6,7-tetrahydrobenzothiophen-6-yl)-N-methyl-carbamate enantiomer 1 (1a) and enantiomer 2 (1b)

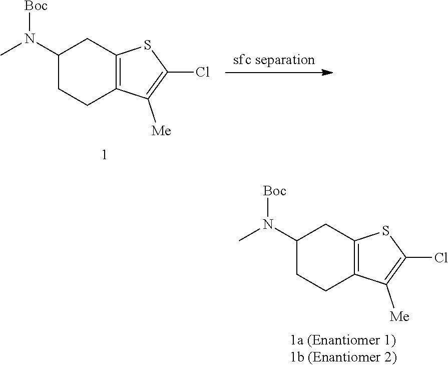 [SMILES: Cc1c(Cl)sc2c1CCC(N(C)C(=O)OC(C)(C)C)C2 ; Cc1c(Cl)sc2c1CCC(N(C)C(=O)OC(C)(C)C)C2]

Racemic tert-butyl N-(2-chloro-3-methyl-4,5,6,7-tetrahydrobenzothiophen-6-yl)-N-methyl-carbamate (1) (130 mg) was separated by SFC (column: DAICEL CHIRALPAK AD (250 mm*30 mm, 10 μm); mobile phase: [CO₂-MeOH (0.1% NH₃H₂O)]; B %:10%, isocratic elution mode) to give tert-butyl N-(2-chloro-3-methyl-4,5,6,7-tetrahydrobenzothiophen-6-yl)-N-methyl-carbamate enantiomer 1 (1a) (60 mg, 189.96 μmol, 46.15% yield) as yellow oil and tert-butyl N-(2-chloro-3-methyl-4,5,6,7-tetrahydrobenzothiophen-6-yl)-N-methyl-carbamate enantiomer 2 (1b) (55 mg, 174.13 μmol, 42.31% yield) as yellow oil.

### Procedure for preparation of 2-chloro-N, 3-dimethyl-4,5,6,7-tetrahydrobenzothiophen-6-amine hydrochloride enantiomer 1 (147)

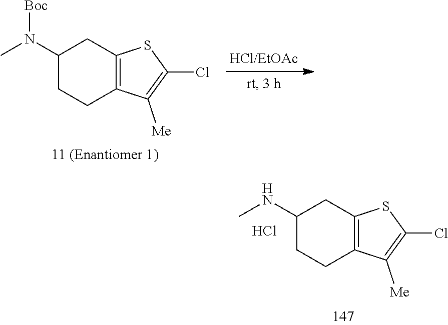 [SMILES: Cc1c(Cl)sc2c1CCC(N(C)C(=O)OC(C)(C)C)C2 ; CNC1CCc2c(sc(Cl)c2C)C1 ; Cl]

To a solution of tert-butyl N-(2-chloro-3-methyl-4,5,6,7-tetrahydrobenzothiophen-6-yl)-N-methyl-carbamate enantiomer 1 (1a) (60 mg, 189.96 μmol, 1 eq) in EtOAc (1 mL) was added HCl/EtOAc (4 M, 1.09 mL, 22.97 eq). The mixture was stirred at 25° C. for 3 hrs. The reaction mixture was concentrated under reduced pressure to remove HCl/EtOAc. The product was triturated with EtOAc at 25° C. for 30 min. The mixture was filtered and the filter cake was dried to afford 2-chloro-N, 3-dimethyl-4,5,6,7-tetrahydrobenzothiophen-6-amine hydrochloride enantiomer 1 (147) (45.31 mg, 177.86 μmol, 93.63% yield) as a white solid. ¹H NMR (400 MHz, METHANOL-d4) δ=3.55 (dddd, J=3.0, 5.4, 8.1, 10.6 Hz, 1H), 3.24-3.16 (m, 1H), 2.88-2.75 (m, 4H), 2.74-2.55 (m, 2H), 2.33-2.24 (m, 1H), 2.05 (s, 3H), 1.94 (dddd, J=6.2, 9.2, 10.2, 13.1 Hz, 1H). LCMS: m/z [M+H]⁺=216.2.

### Procedure for preparation of 2-chloro-N, 3-dimethyl-4,5,6,7-tetrahydrobenzothiophen-6-amine hydrochloride (148)

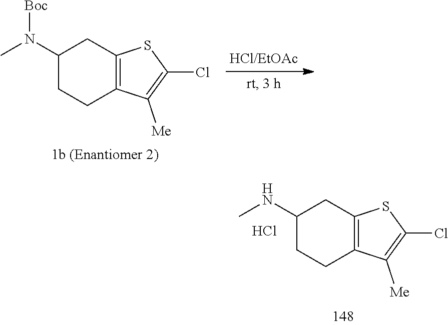 [SMILES: Cc1c(Cl)sc2c1CCC(N(C)C(=O)OC(C)(C)C)C2 ; CNC1CCc2c(sc(Cl)c2C)C1 ; Cl]

To a solution of tert-butyl N-(2-chloro-3-methyl-4,5,6,7-tetrahydrobenzothiophen-6-yl)-N-methyl-carbamate enantiomer 2 (1b) (55 mg, 174.13 μmol, 1 eq) in EtOAc (1 mL) was added HCl/EtOAc (4 M, 916.67 μL, 21.06 eq). The mixture was stirred at 25° C. for 3 hr. The reaction mixture was concentrated under reduced pressure to remove HCl/EtOAc. The product was triturated with EtOAc at 25° C. for 30 min. The mixture was filtered and the filter cake was dried to afford 2-chloro-N, 3-dimethyl-4,5,6,7-tetrahydrobenzothiophen-6-amine hydrochloride enantiomer 2 (148) (44.29 mg, 173.86 μmol, 99.84% yield) as a white solid. ¹H NMR (400 MHz, METHANOL-d4) δ=3.55 (dddd, J=2.8, 5.3, 8.1, 10.6 Hz, 1H), 3.20 (dd, J=4.6, 15.9 Hz, 1H), 2.86-2.75 (m, 4H), 2.74-2.55 (m, 2H), 2.34-2.24 (m, 1H), 2.04 (s, 3H), 2.01-1.87 (m, 1H). LCMS: m/z [M+H]⁺=216.1.

### Synthetic scheme for preparation N, 1,5-trimethyl-6,7-dihydro-4H-2-benzothiophen-5-amine hydrochloride enantiomer 1 (149) and enantiomer 2 (150)

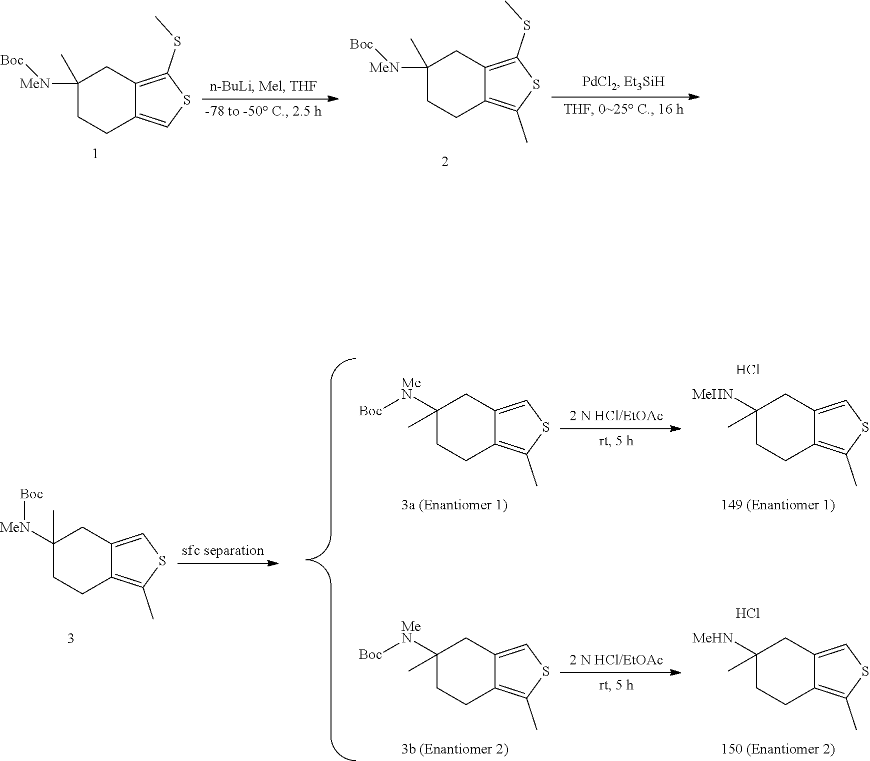

### Procedure for preparation of tert-butyl N-(1,5-dimethyl-3-methylsulfanyl-6,7-dihydro-4H-2-benzothiophen-5-yl)-N-methyl-carbamate (2)

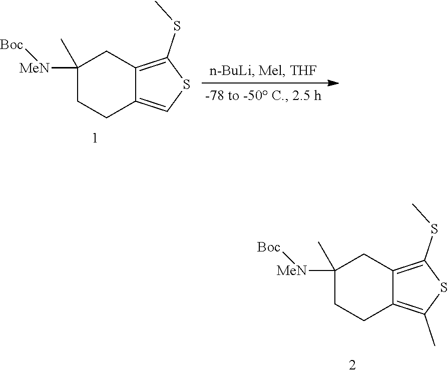 [SMILES: CSc1scc2c1CC(C)(N(C)C(=O)OC(C)(C)C)CC2 ; CSc1sc(C)c2c1CC(C)(N(C)C(=O)OC(C)(C)C)CC2]

To a solution of tert-butyl N-methyl-N-(5-methyl-3-methylsulfanyl-6,7-dihydro-4H-2-benzothiophen-5-yl)carbamate (1) (650 mg, 1.98 mmol, 1 eq) in THF (10 mL) was added dropwise n-BuLi (2.5 M, 1.59 mL, 2 eq) at −78° C. over 0.25 hr. After addition, the mixture was stirred at this temperature for 0.25 hr, and then CH₃I (422.56 mg, 2.98 mmol, 185.33 μL, 1.5 eq) was added dropwise at −78° C. The resulting mixture was stirred at −78~−50° C. for 2 hr. The reaction mixture was poured into NH₄Cl aq. saturated solution (20 mL) and extracted with EtOAc (20 mL*3). The combined organic phases were washed with brine (10 mL), dried over anhydrous sodium sulfate, filtered and concentrated under vacuum to give tert-butyl N-(1,5-dimethyl-3-methylsulfanyl-6,7-dihydro-4H-2-benzothiophen-5-yl)-N-methyl-carbamate (2) (610 mg, crude) as yellow oil. ¹H NMR (400 MHz, CHLOROFORM-d) δ 3.34 (dd, J=2.4, 17.1 Hz, 1H), 3.03-2.93 (m, 1H), 2.71 (s, 3H), 2.60-2.50 (m, 1H), 2.46-2.34 (m, 1H), 2.32 (s, 3H), 2.28 (s, 3H), 1.65-1.58 (m, 2H), 1.47 (s, 9H), 1.43 (s, 3H)

### Procedure for preparation of tert-butyl N-(1,5-dimethyl-6,7-dihydro-4H-2-benzothiophen-5-yl)-N-methyl-carbamate (3)

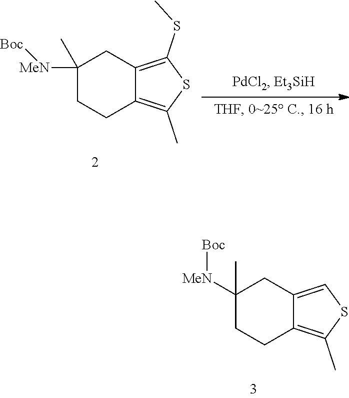 [SMILES: CSc1sc(C)c2c1CC(C)(N(C)C(=O)OC(C)(C)C)CC2 ; Cc1scc2c1CCC(C)(N(C)C(=O)OC(C)(C)C)C2]

To a solution of tert-butyl N-(1,5-dimethyl-3-methylsulfanyl-6,7-dihydro-4H-2-benzothiophen-5-yl)-N-methyl-carbamate (2) (610 mg, 1.79 mmol, 1 eq) in THF (8 mL) was added PdCl₂ (31.67 mg, 178.61 μmol, 0.1 eq) and Et₃SiH (311.52 mg, 2.68 mmol, 427.91 μL, 1.5 eq) at 0° C. The mixture was stirred at 25° C. for 16 hr. The mixture was filtered, the filtrate was poured into aq. sat. NaHCO₃ (10 mL) and extracted with EtOAc (10 mL*3). The combined organic phases was washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~20% Ethyl acetate/Petroleum ether gradient @40 mL/min) to give tert-butyl N-(1,5-dimethyl-6,7-dihydro-4H-2-benzothiophen-5-yl)-N-methyl-carbamate (3) (300 mg) as a yellow solid. LCMS: m/z [M-NMeBoc]⁺=165.2

### Procedure for preparation of tert-butyl N-(1,5-dimethyl-6,7-dihydro-4H-2-benzothiophen-5-yl)-N-methyl-carbamate enantiomer 1 (3a) and enantiomer 2 (3b)

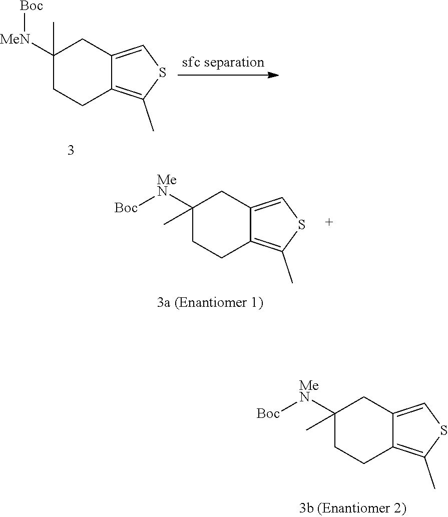 [SMILES: Cc1scc2c1CCC(C)(N(C)C(=O)OC(C)(C)C)C2 ; Cc1scc2c1CCC(C)(N(C)C(=O)OC(C)(C)C)C2 ; Cc1scc2c1CCC(C)(N(C)C(=O)OC(C)(C)C)C2]

Racemic tert-butyl N-(1,5-dimethyl-6,7-dihydro-4H-2-benzothiophen-5-yl)-N-methyl-carbamate (3) (200 mg) was separated by SFC (column: DAICEL CHIRALCEL OD (250 mm*50 mm, 10 μm); mobile phase: [Hexane-IPA (0.1% NH₃)]; B %:10%, isocratic elution mode) to give tert-butyl N-(1,5-dimethyl-6,7-dihydro-4H-2-benzothiophen-5-yl)-N-methyl-carbamate enantiomer 1 (3a) (75 mg) as a yellow oil and tert-butyl N-(1,5-dimethyl-6,7-dihydro-4H-2-benzothiophen-5-yl)-N-methyl-carbamate enantiomer 2 (3b) (85 mg) as a yellow oil.

### Procedure for preparation of N, 1,5-trimethyl-6,7-dihydro-4H-2-benzothiophen-5-amine hydrochloride enantiomer 1 (149)

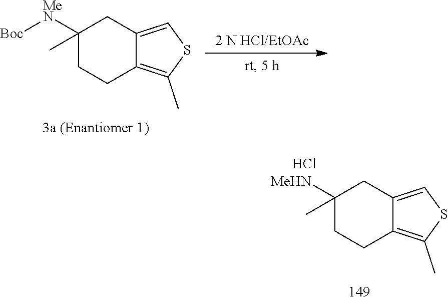 [SMILES: Cc1scc2c1CCC(C)(N(C)C(=O)OC(C)(C)C)C2 ; Cl ; CNC1(C)CCc2c(csc2C)C1]

To a solution of tert-butyl N-(1,5-dimethyl-6,7-dihydro-4H-2-benzothiophen-5-yl)-N-methyl-carbamate enantiomer 1 (3a) (75 mg, 253.86 μmol, 1 eq) in EtOAc (1 mL) was added HCl/EtOAc (4 M, 882.35 μL, 13.90 eq). The mixture was stirred at 25° C. for 5 hr. The reaction mixture was concentrated under reduced pressure to remove HCl/EtOAc. The product was triturated with EtOAc at 25° C. for 30 min. The mixture was filtred, the filter cake was dried to give N, 1,5-trimethyl-6,7-dihydro-4H-2-benzothiophen-5-amine hydrochloride enantiomer 1 (149) (53.91 mg, 230.26 μmol, 90.70% yield) as a white solid. ¹H NMR (400 MHz, METHANOL-d4) δ 6.86 (s, 1H), 2.98-2.86 (m, 2H), 2.80-2.64 (m, 5H), 2.33 (s, 3H), 2.08-1.99 (m, 2H), 1.35 (s, 3H). LCMS: m/z [M+H]⁺=196.3

### Procedure for preparation of N, 1,5-trimethyl-6,7-dihydro-4H-2-benzothiophen-5-amine hydrochloride enantiomer 2 (150)

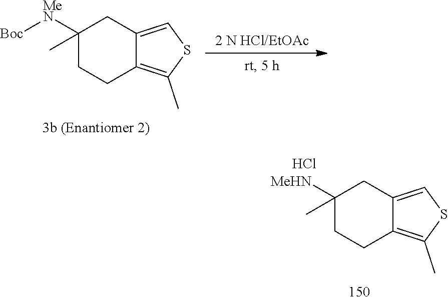 [SMILES: Cc1scc2c1CCC(C)(N(C)C(=O)OC(C)(C)C)C2 ; Cl ; CNC1(C)CCc2c(csc2C)C1]

To a solution of tert-butyl N-(1,5-dimethyl-6,7-dihydro-4H-2-benzothiophen-5-yl)-N-methyl-carbamate enantiomer 2 (3b) (85 mg, 287.71 μmol, 1 eq) in EtOAc (1 mL) was added HCl/EtOAc (4 M, 1.00 mL, 13.90 eq). The mixture was stirred at 25° C. for 5 hr. The reaction mixture was concentrated under reduced pressure to remove HCl/EtOAc. The product was triturated with EtOAc at 25° C. for 30 min. The mixture was filtered, the filter cake was dried to give N, 1,5-trimethyl-6,7-dihydro-4H-2-benzothiophen-5-amine hydrochloride enantiomer 2 (150) (44.58 mg, 192.33 μmol, 66.85% yield) as a white solid. ¹H NMR (400 MHz, METHANOL-d4) δ 6.86 (s, 1H), 2.99-2.84 (m, 2H), 2.80-2.62 (m, 5H), 2.33 (s, 3H), 2.10-1.97 (m, 2H), 1.35 (s, 3H). LCMS: m/z [M+H]⁺=196.2

### Synthetic scheme for the preparation of 2-chloro-N-methyl-4,5,6,7-tetrahydrobenzothiophen-5-amine hydrochloride enantiomer 1 (151) and enantiomer 2 (152)

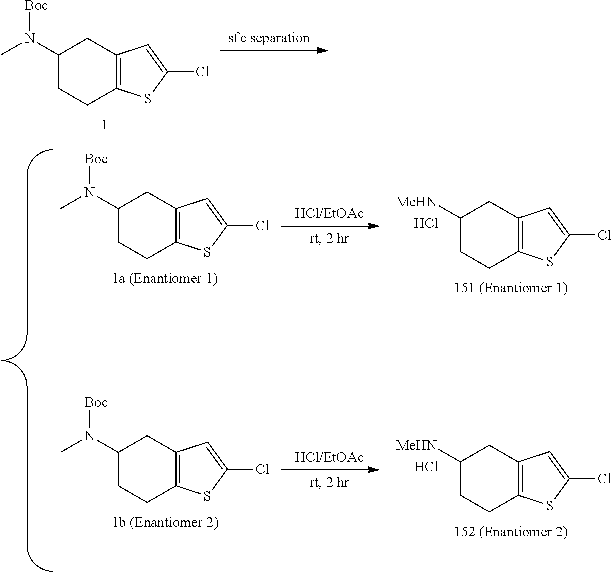

### Procedure for preparation of tert-butyl N-(2-chloro-4,5,6,7-tetrahydrobenzothiophen-5-yl)-N-methyl-carbamate enantiomer 1 (1a) and enantiomer 2 (1b)

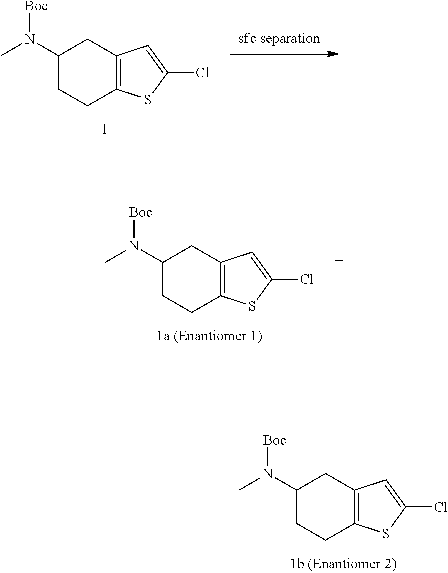 [SMILES: CN(C(=O)OC(C)(C)C)C1CCc2sc(Cl)cc2C1 ; CN(C(=O)OC(C)(C)C)C1CCc2sc(Cl)cc2C1 ; CN(C(=O)OC(C)(C)C)C1CCc2sc(Cl)cc2C1]

Racemic tert-butyl N-(2-chloro-4,5,6,7-tetrahydrobenzothiophen-5-yl)-N-methyl-carbamate (1) (194 mg) was seperated by SFC (column: DAICEL CHIRALPAK AD (250 mm*50 mm, 10 μm); mobile phase: [CO₂-MeOH (0.1% NH₃H₂O)]; B %:15%, isocratic elution mode) to give tert-butyl N-(2-chloro-4,5,6,7-tetrahydrobenzothiophen-5-yl)-N-methyl-carbamate enantiomer 1 (1a) (100 mg, 1st peak in SFC) and enantiomer 2 (1b) (83 mg, 2nd peak in SFC).

### Procedure for preparation of 2-chloro-N-methyl-4,5,6,7-tetrahydrobenzothiophen-5-amine hydrochloride enantiomer 1 (151)

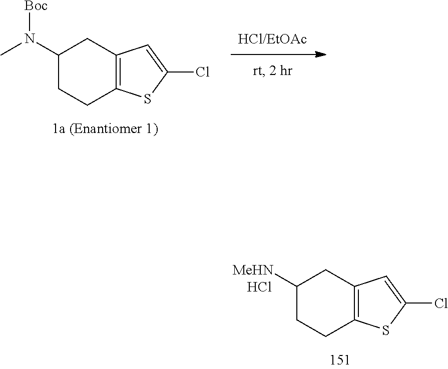 [SMILES: CN(C(=O)OC(C)(C)C)C1CCc2sc(Cl)cc2C1 ; Cl ; CNC1CCc2sc(Cl)cc2C1]

To a solution of tert-butyl N-(2-chloro-4,5,6,7-tetrahydrobenzothiophen-5-yl)-N-methyl-carbamate enantiomer 1 (1a) (100 mg, 331.31 μmol, 1 eq) in EtOAc (1 mL) was added HCl/EtOAc (4 M, 3.45 mL, 41.63 eq). The mixture was stirred at 25° C. for 2 hr. The reaction mixture was concentrated under reduced pressure to remove EtOAc. The crude product was triturated with EtOAc at 25° C. for 30 min. Compound 2-chloro-N-methyl-4,5,6,7-tetrahydrobenzothiophen-5-amine hydrochloride enantiomer 1 (151) (75.89 mg, 318.63 μmol, 96.17% yield) was obtained as a white solid. ¹H NMR (400 MHz, METHANOL-d₄) δ=6.71 (s, 1H), 3.56-3.45 (m, 1H), 3.10 (br dd, J=5.1, 15.6 Hz, 1H), 2.91-2.83 (m, 2H), 2.78 (s, 3H), 2.65 (br dd, J=9.7, 15.6 Hz, 1H), 2.36-2.27 (m, 1H), 2.03-1.88 (m, 1H). LCMS: m/z [M+H]⁺=202.1.

### Procedure for preparation of 2-chloro-N-methyl-4,5,6,7-tetrahydrobenzothiophen-5-amine hydrochloride enantiomer 2 (152)

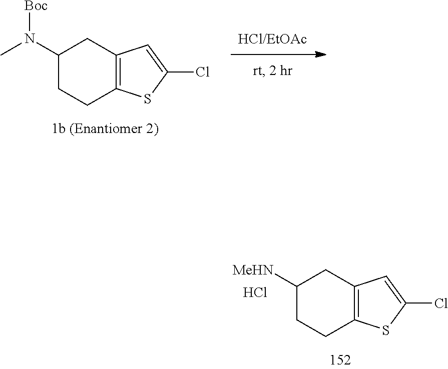 [SMILES: CN(C(=O)OC(C)(C)C)C1CCc2sc(Cl)cc2C1 ; Cl ; CNC1CCc2sc(Cl)cc2C1]

To a solution of tert-butyl N-(2-chloro-4,5,6,7-tetrahydrobenzothiophen-5-yl)-N-methyl-carbamate enantiomer 2 (1b) (83 mg, 274.99 μmol, 1 eq) in EtOAc (1 mL) was added HCl/EtOAc (4 M, 2.86 mL, 41.63 eq). The mixture was stirred at 25° C. for 2 hr. The reaction mixture was concentrated under reduced pressure to remove EtOAc. The crude product was triturated with EtOAc at 25° C. for 30 min. Compound 2-chloro-N-methyl-4,5,6,7-tetrahydrobenzothiophen-5-amine hydrochloride enantiomer 2 (152) (37.66 mg, 158.12 μmol, 57.50% yield) was obtained as a white solid. ¹H NMR (400 MHz, METHANOL-d₄) δ=6.71 (s, 1H), 3.56-3.44 (m, 1H), 3.10 (br dd, J=5.0, 16.0 Hz, 1H), 2.93-2.83 (m, 2H), 2.78 (s, 3H), 2.70-2.60 (m, 1H), 2.36-2.27 (m, 1H), 2.03-1.89 (m, 1H). LCMS: m/z [M+H]⁺=202.1

### Synthetic Scheme for preparation of N-methyl-5-(trifluoromethyl)-3,4-dihydro-2H-thieno[3,4-b]pyran-3-amine hydrochloride (153)

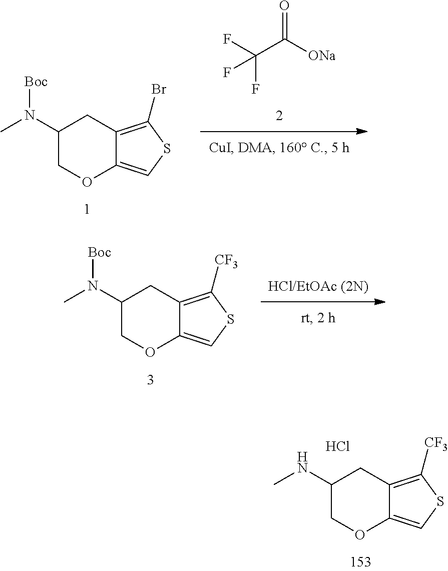

### Procedure for preparation of tert-butyl N-methyl-N-[5-(trifluoromethyl)-3,4-dihydro-2H-thieno[3,4-b]pyran-3-yl]carbamate (3)

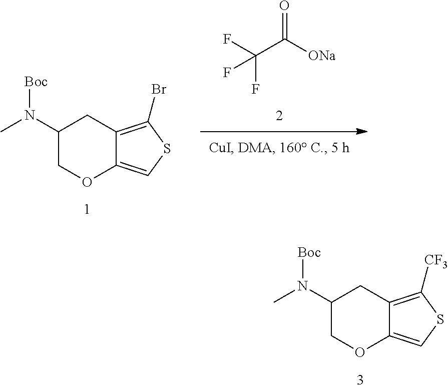

To a solution of tert-butyl N-(5-bromo-3,4-dihydro-2H-thieno[3,4-b]pyran-3-yl)-N-methyl-carbamate (1) (100 mg, 287.15 μmol, 1 eq) in DMA (1 mL) was added sodium 2,2,2-trifluoroacetate (390.53 mg, 2.87 mmol, 10 eq) and CuI (109.37 mg, 574.29 μmol, 2 eq). The mixture was stirred at 160° C. for 5 h. The reaction mixture was filtered and concentrated under reduced pressure to give a residue. The residue was purified by prep-TLC (SiO2, Petroleum ether:Ethyl acetate=5:1, Rf=0.45). Compound tert-butyl N-methyl-N-[5-(trifluoromethyl)-3,4-dihydro-2H-thieno[3,4-b]pyran-3-yl]carbamate (3) (45 mg, 133.39 μmol, 46.45% yield) was obtained as a white solid. LCMS: m/z [M+H-tBu]⁺=282.0.

### Procedure for preparation of N-methyl-5-(trifluoromethyl)-3,4-dihydro-2H-thieno[3,4-b]pyran-3-amine hydrochloride (153)

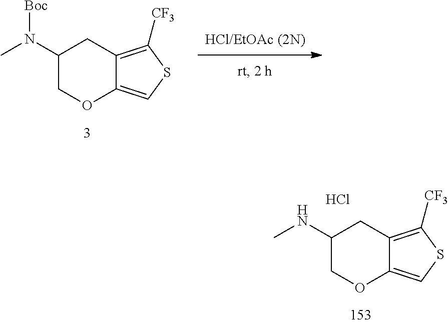 [SMILES: CN(C(=O)OC(C)(C)C)C1COc2csc(C(F)(F)F)c2C1 ; CNC1COc2csc(C(F)(F)F)c2C1 ; Cl]

To a solution of tert-butyl N-methyl-N-[5-(trifluoromethyl)-3,4-dihydro-2H-thieno[3,4-b]pyran-3-yl]carbamate (3) (45 mg, 133.39 μmol, 1 eq) in EtOAc (1 mL) was added HCl/EtOAc (4 M, 1 mL, 29.99 eq). The mixture was stirred at 25° C. for 2 hr. The reaction mixture was concentrated under reduced pressure to give a residue. The crude product was triturated with EtOAc (10 mL) at 25° C. for 30 min and filtered. Compound N-methyl-5-(trifluoromethyl)-3,4-dihydro-2H-thieno[3,4-b]pyran-3-amine hydrochloride (153) (10 mg, 36.54 μmol, 27.39% yield) was obtained as a white solid. LCMS: m/z [M+H]⁺=238.0. ¹H NMR (400 MHz, METHANOL-d4) δ=6.94 (s, 1H), 4.58-4.51 (m, 1H), 4.26 (dd, J=1.0, 12.6 Hz, 1H), 3.81 (br d, J=2.1 Hz, 1H), 3.46-3.37 (m, 1H), 3.28-3.19 (m, 1H), 2.82 (s, 3H).

### Synthetic scheme for the preparation of 3-cyclopropyl-N-methyl-4,5,6,7-tetrahydro-2-benzothiophen-5-amine hydrochloride enantiomer 1 (154) and enantiomer 2 (155)

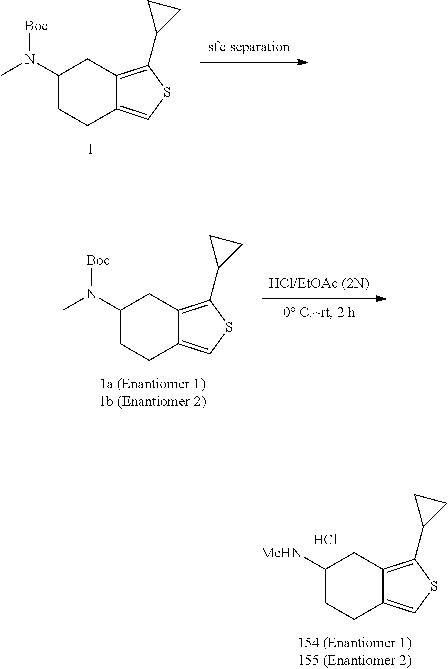 [SMILES: CN(C(=O)OC(C)(C)C)C1CCc2csc(C3CC3)c2C1 ; CN(C(=O)OC(C)(C)C)C1CCc2csc(C3CC3)c2C1 ; CNC1CCc2csc(C3CC3)c2C1 ; Cl]

### Procedure for preparation of tert-butyl N-(3-cyclopropyl-4,5,6,7-tetrahydro-2-benzothiophen-5-yl)-N-methyl-carbamate enantiomer 1 (1a) and enantiomer 2 (1b)

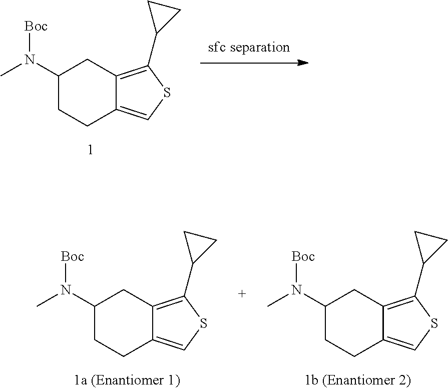 [SMILES: CN(C(=O)OC(C)(C)C)C1CCc2csc(C3CC3)c2C1 ; CN(C(=O)OC(C)(C)C)C1CCc2csc(C3CC3)c2C1 ; CN(C(=O)OC(C)(C)C)C1CCc2csc(C3CC3)c2C1]

Racemic tert-butyl N-(3-cyclopropyl-4,5,6,7-tetrahydro-2-benzothiophen-5-yl)-N-methyl-carbamate (1) (0.2 g) was separated by SFC (column: DAICEL CHIRALPAK AY (250 mm*50 mm, 10 μm); mobile phase: [Hexane-EtOH]; B %:10%, isocratic elution mode) to enantiomers (1a) and (1b). tert-Butyl N-(3-cyclopropyl-4,5,6,7-tetrahydro-2-benzothiophen-5-yl)-N-methyl-carbamate enantiomer 1 (1a) was further purified by prep-HPLC (column: Phenomenex Luna C18 150*25 mm*10 μm; mobile phase: [water(FA)-ACN]; gradient:67%-97% B over 10 min) to yield pure tert-butyl N-(3-cyclopropyl-4,5,6,7-tetrahydro-2-benzothiophen-5-yl)-N-methyl-carbamate enantiomer 1 (1a) (60 mg, 195.15 mol) as a colorless oil. tert-Butyl N-(3-cyclopropyl-4,5,6,7-tetrahydro-2-benzothiophen-5-yl)-N-methyl-carbamate enantiomer 2 (1b) was also further purified by prep-HPLC (column: Phenomenex Luna C18 150*25 mm*10 μm; mobile phase: [water(FA)-ACN]; gradient:67%-97% B over 10 min) to yield pure tert-butyl N-(3-cyclopropyl-4,5,6,7-tetrahydro-2-benzothiophen-5-yl)-N-methyl-carbamate enantiomer 2 (1b) (50 mg, 162.63 mol) as a colorless oil.

### Procedure for preparation of 3-cyclopropyl-N-methyl-4,5,6,7-tetrahydro-2-benzothiophen-5-amine hydrochloride enantiomer 1 (154)

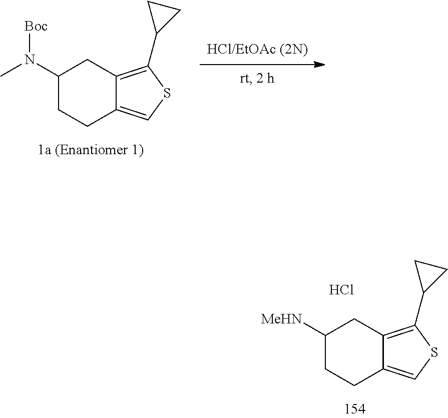 [SMILES: CN(C(=O)OC(C)(C)C)C1CCc2csc(C3CC3)c2C1 ; CNC1CCc2csc(C3CC3)c2C1 ; Cl]

To a solution of tert-butyl N-(3-cyclopropyl-4,5,6,7-tetrahydro-2-benzothiophen-5-yl)-N-methyl-carbamate enantiomer 1 (1a) (60 mg, 195 μmol, 1 eq) in EtOAc (2 mL) was added HCl/EtOAc (4 M, 2 mL, 40.9 eq). The mixture was stirred at 25° C. for 2 hr. The reaction mixture was filtered and the cake was washed with EtOAc (10 mL). Compound 3-cyclopropyl-N-methyl-4,5,6,7-tetrahydro-2-benzothiophen-5-amine hydrochloride enantiomer 1 (154) (33.7 mg, 132.62 μmol, 67.96% yield) was obtained as a white solid. ¹H NMR (400 MHz, METHANOL-d4) δ=6.73 (s, 1H), 3.53-3.42 (m, 1H), 3.34 (br d, J=6.4 Hz, 1H), 2.96-2.87 (m, 1H), 2.84-2.62 (m, 5H), 2.29-2.18 (m, 1H), 2.04-1.92 (m, 1H), 1.89-1.76 (m, 1H), 1.04-0.94 (m, 2H), 0.67-0.59 (m, 2H). LCMS: m/z [M+H]⁺=208.1

### Procedure for preparation of 3-cyclopropyl-N-methyl-4,5,6,7-tetrahydro-2-benzothiophen-5-amine hydrochloride enantiomer 2 (155)

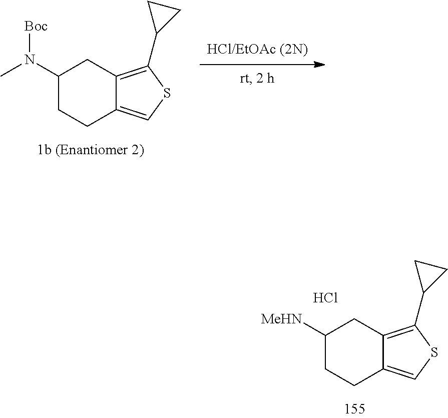 [SMILES: CN(C(=O)OC(C)(C)C)C1CCc2csc(C3CC3)c2C1 ; CNC1CCc2csc(C3CC3)c2C1 ; Cl]

To a solution of tert-butyl N-(3-cyclopropyl-4,5,6,7-tetrahydro-2-benzothiophen-5-yl)-N-methyl-carbamate enantiomer 2 (1b) (50 mg, 162.63 μmol, 1 eq) in EtOAc (2 mL) was added HCl/EtOAc (4 M, 2 mL, 49.19 eq). The mixture was stirred at 25° C. for 2 hr. The reaction mixture was filtered and the cake was washed with EtOAc (10 mL). Compound 3-cyclopropyl-N-methyl-4,5,6,7-tetrahydro-2-benzothiophen-5-amine hydrochloride enantiomer 2 (155) (37.12 mg, 145.29 μmol, 89.34% yield) was obtained as a white solid. ¹H NMR (400 MHz, METHANOL-d4) δ=6.73 (s, 1H), 3.52-3.42 (m, 1H), 3.34 (br d, J=5.9 Hz, 1H), 2.96-2.86 (m, 1H), 2.81 (s, 3H), 2.79-2.63 (m, 2H), 2.31-2.17 (m, 1H), 2.03-1.91 (m, 1H), 1.89-1.76 (m, 1H), 1.06-0.92 (m, 2H), 0.70-0.57 (m, 2H). LCMS: m/z [M+H]⁺=208.1

### Synthetic scheme for preparation of N, N-dimethyl-4, 5, 6, 7-tetrahydro-2-benzothiophen-5-amine hydrochloride enantiomer 1 (156) and enantiomer 2 (157)

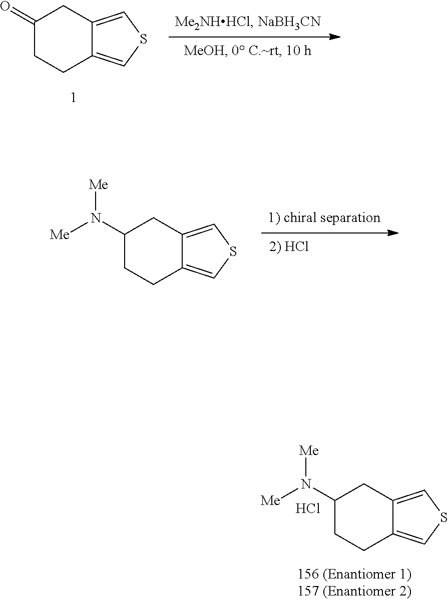 [SMILES: O=C1CCc2cscc2C1 ; CN(C)C1CCc2cscc2C1 ; CN(C)C1CCc2cscc2C1 ; Cl]

### Procedure for preparation of N,N-dimethyl-4,5,6,7-tetrahydro-2-benzothiophen-5-amine hydrochloride enantiomer 1 (156) and enantiomer 2 (157)

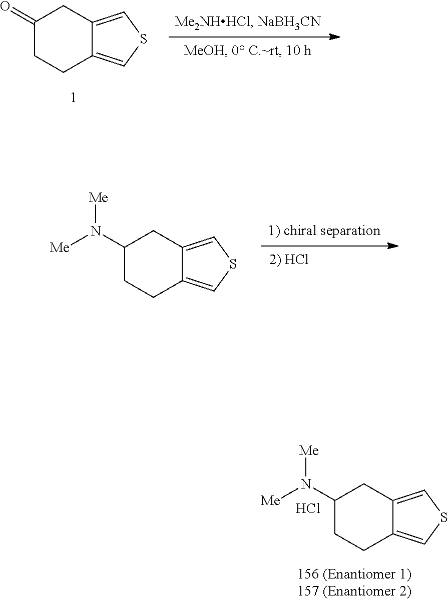 [SMILES: O=C1CCc2cscc2C1 ; CN(C)C1CCc2cscc2C1 ; CN(C)C1CCc2cscc2C1 ; Cl]

To a solution of 6, 7-dihydro-4H-2-benzothiophen-5-one (1) (700 mg, 4.60 mmol, 1 eq) in MeOH (20 mL) was added a solution of N-methylmethanamine hydrochloride (1.13 g, 13.8 mmol, 3 eq) at 0° C., then was added NaBH₃CN (1.44 g, 23.0 mmol, 5 eq) slowly at 0° C. The mixture was allowed to warm up to 25° C. and stirred for 10 hr. The reaction mixture was diluted with water (10 mL) and concentrated under vacuum to remove the organic solvent. The aqueous phase was extracted with EtOAc (30 mL*3). The combined organic phase was washed with brine (30 mL), dried over anhydrous sodium sulfate, filtered and concentrated under vacuum to give a residue. The residue was purified by flash silica gel chromatography (ISCO®; 12 g SepaFlash® Silica Flash Column, eluent 0-100% Ethyl acetate/Petroleum ether gradient @40 mL/min) to afford N, N-dimethyl-4, 5, 6, 7-tetrahydro-2-benzothiophen-5-amine. The racemic product was subjected to prep-Chiral-SFC (column: DAICEL CHIRALCEL OX (250 mm*30 mm, 10 μm); mobile phase: [0.1% NH₃H₂O EtOH]; B %:45%, isocratic elution mode) to afford the 1^{st} enantiomer (RT=1.851 min) and the 2^{nd} enantiomer (RT=2.115 min). Fractions contained enantiomer 1 concentrated and the residue treated with 4N HCl/EtOAc (1 mL). The mixture was stirred at 25° C. for 0.5 hour. The mixture was filtered and filter cake was collected. Compound N, N-dimethyl-4, 5, 6, 7-tetrahydro-2-benzothiophen-5-amine hydrochloride enantiomer 1 (156) (27.11 mg, 123.33 μmol, 2.68% yield) was obtained as a white solid. ¹H NMR (400 MHz, METHANOL-d₄) δ 7.11 (br s, 1H), 7.04 (d, J=1.2 Hz, 1H), 3.59 (ddt, J=2.6, 5.1, 11.5 Hz, 1H), 3.27 (br d, J=5.0 Hz, 1H), 3.11-3.02 (m, 1H), 2.97-2.87 (m, 7H), 2.84-2.74 (m, 1H), 2.35-2.24 (m, 1H), 1.91 (dq, J=5.3, 12.0 Hz, 1H). LCMS: m/z [M+H]⁺=182.2. Fractions contained enantiomer 2 concentrated and the residue treated with 4N HCl/EtOAc (1 mL). The mixture was stirred at 25° C. for 0.5 hour and evaporated. Compound N, N-dimethyl-4, 5, 6, 7-tetrahydro-2-benzothiophen-5-amine hydrochloride enantiomer 2 (157) (38 mg) was isolated as an oil. ¹H NMR (400 MHz, METHANOL-d₄) δ 7.15-7.12 (m, 1H), 7.08-7.04 (m, 1H), 3.63 (ddt, J=2.7, 5.0, 11.5 Hz, 1H), 3.29 (br d, J=5.1 Hz, 1H), 3.13-3.04 (m, 1H), 2.97 (d, J=2.0 Hz, 6H), 2.88-2.74 (m, 1H), 2.36-2.26 (m, 1H), 1.99-1.87 (m, 1H). LCMS: m/z [M+H]⁺=182.2.

### Synthetic scheme for preparation of N-(cyclopropylmethyl)-4,5,6,7-tetrahydro-2-benzothiophen-5-amine hydrochloride enantiomer 1 (158) and enantiomer 2 (159)

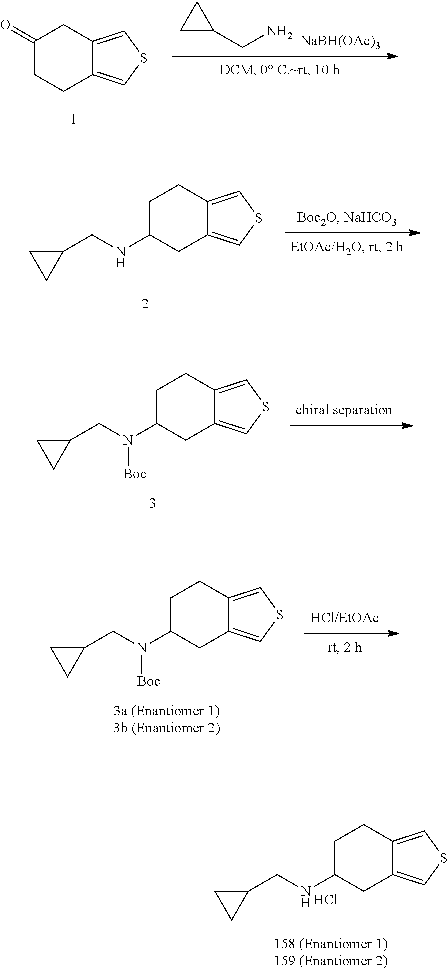

### Procedure for preparation of N-(cyclopropylmethyl)-4,5,6,7-tetrahydro-2-benzothiophen-5-amine (2)

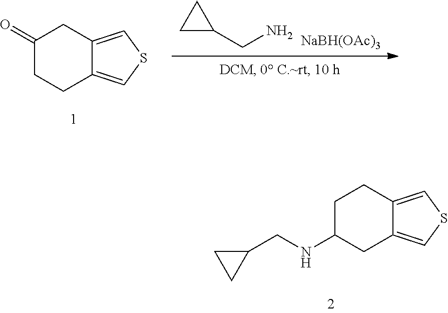

To a solution of 6,7-dihydro-4H-2-benzothiophen-5-one (1) (700 mg, 4.60 mmol, 1 eq) in DCM (10 mL) was added a solution of cyclopropylmethanamine (981.21 mg, 13.80 mmol, 3 eq) in DCM (10 mL) at 0° C., then was added NaBH(OAc)₃ (1.95 g, 9.20 mmol, 2 eq) slowly at 0° C. The mixture was stirred at 25° C. for 10 hr. The reaction mixture was diluted with H₂O (30 mL) and extracted with DCM (20 mL*2). The combined organic layers were washed with brine (30 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~100% A/Petroleum ether gradient (A: Ethyl acetate/NH₃·MeOH (7N)=100/1) @40 mL/min) to give N-(cyclopropylmethyl)-4,5,6,7-tetrahydro-2-benzothiophen-5-amine (2) (400 mg, 1.93 mmol, 41.95% yield) as a colorless oil.

### Procedure for preparation of tert-butyl N-(cyclopropylmethyl)-N-(4,5,6,7-tetrahydro-2-benzothiophen-5-yl) carbamate enantiomer 1 (3a) and enantiomer 2 (3b)

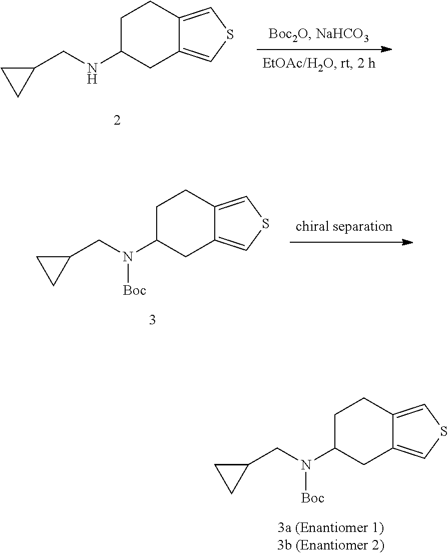 [SMILES: c1scc2c1CCC(NCC1CC1)C2 ; CC(C)(C)OC(=O)N(CC1CC1)C1CCc2cscc2C1 ; CC(C)(C)OC(=O)N(CC1CC1)C1CCc2cscc2C1]

To a solution of N-(cyclopropylmethyl)-4,5,6,7-tetrahydro-2-benzothiophen-5-amine (2) (400 mg, 1.93 mmol, 1 eq) in EtOAc (5 mL) and H₂O (5 mL) was added NaHCO₃ (324.14 mg, 3.86 mmol, 2 eq) and Boc₂O (842.10 mg, 3.86 mmol, 886.42 μL, 2 eq). The mixture was stirred at 25° C. for 2 hr. The reaction mixture was diluted with H₂O (10 mL) and extracted with EtOAc (10 mL*2). The combined organic layers were washed with brine (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~5% Ethyl acetate/Petroleum ether gradient @30 mL/min) to give tert-butyl N-(cyclopropylmethyl)-N-(4,5,6,7-tetrahydro-2-benzothiophen-5-yl) carbamate (3) (550 mg, 1.79 mmol, 92.73% yield) as a colorless oil. This product was separated to its enantiomers by SFC (column: DAICEL CHIRALPAK IC (250 mm*30 mm, 5 μm); mobile phase: [0.1% NH₃H₂O IPA]; B %: 15%-15%, 3.8 min) to afford 1^{st} enantiomer 3a (RT=0.747 min, 200 mg, 650 mol) and the 2^{nd} enantiomer 3b (RT=0.838 min, 230 mg, 748.09 mol) both as yellow oils.

### Procedure for preparation of N-(cyclopropylmethyl)-4,5,6,7-tetrahydro-2-benzothiophen-5-amine hydrochloride enantiomer 1 (158)

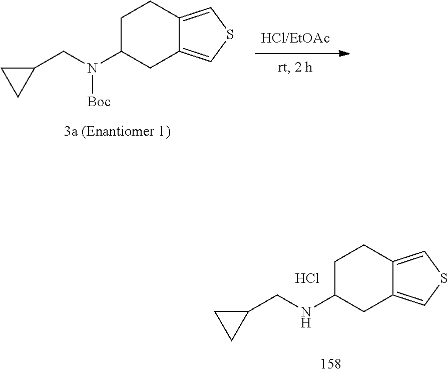 [SMILES: CC(C)(C)OC(=O)N(CC1CC1)C1CCc2cscc2C1 ; c1scc2c1CCC(NCC1CC1)C2 ; Cl]

To a solution of tert-butyl N-(cyclopropylmethyl)-N-(4,5,6,7-tetrahydro-2-benzothiophen-5-yl) carbamate enantiomer 1 (3a) (200 mg, 650 μmol, 1 eq) in EtOAc (2 mL) was added HCl/EtOAc (4 M, 5.26 mL, 32.36 eq). The mixture was stirred at 25° C. for 2 hr. The reaction mixture was filtered and the cake was washed with EtOAc (10 mL) to afford N-(cyclopropylmethyl)-4,5,6,7-tetrahydro-2-benzothiophen-5-amine hydrochloride enantiomer 1 (158) (151.82 mg, 619.62 μmol, 95.25% yield) as a white solid. ¹H NMR (400 MHz, METHANOL-d₄) δ=7.09 (br s, 1H), 7.03 (br s, 1H), 3.52 (ddt, J=2.8, 5.1, 10.9 Hz, 1H), 3.35 (br d, J=15.7 Hz, 1H), 3.08-2.96 (m, 3H), 2.85-2.69 (m, 2H), 2.35-2.24 (m, 1H), 1.84 (dq, J=5.4, 11.9 Hz, 1H), 1.22-1.07 (m, 1H), 0.80-0.71 (m, 2H), 0.51-0.41 (m, 2H). LCMS: m/z [M+H]⁺=208.5.

### Procedure for preparation of N-(cyclopropylmethyl)-4,5,6,7-tetrahydro-2-benzothiophen-5-amine hydrochloride enantiomer 2 (159)

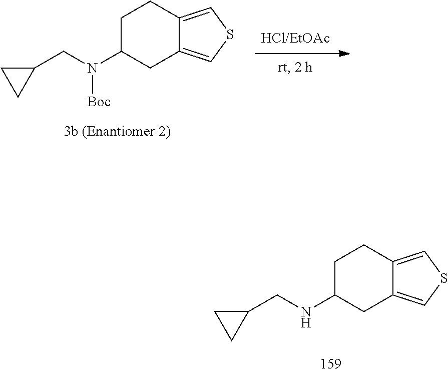 [SMILES: CC(C)(C)OC(=O)N(CC1CC1)C1CCc2cscc2C1 ; c1scc2c1CCC(NCC1CC1)C2]

To a solution of tert-butyl-N-(cyclopropylmethyl)-N-(4,5,6,7-tetrahydro-2-benzothiophen-5-yl) carbamate enantiomer 2 (3b) (230 mg, 748 μmol, 1 eq) in EtOAc (2 mL) was added HCl/EtOAc (4 M, 5 mL, 26.73 eq). The mixture was stirred at 25° C. for 2 hr. The reaction mixture was filtered and the cake was washed with EtOAc (10 mL) to afford N-(cyclopropylmethyl)-4,5,6,7-tetrahydro-2-benzothiophen-5-amine hydrochloride enantiomer 2 (159) (154.95 mg, 635.57 μmol, 84.96% yield) as a white solid. ¹H NMR (400 MHz, METHANOL-d₄) δ=7.09 (br s, 1H), 7.04 (br s, 1H), 3.52 (ddt, J=2.9, 5.2, 10.9 Hz, 1H), 3.36 (br d, J=5.3 Hz, 1H), 3.08-2.96 (m, 3H), 2.86-2.68 (m, 2H), 2.38-2.25 (m, 1H), 1.84 (dq, J=5.4, 11.9 Hz, 1H), 1.20-1.07 (m, 1H), 0.79-0.70 (m, 2H), 0.49-0.39 (m, 2H). LCMS: m/z [M+H]⁺=208.5.

### Synthetic scheme for the preparation of N-cyclobutyl-4,5,6,7-tetrahydro-2-benzothiophen-5-amine hydrochloride enantiomer 1 (160) and enantiomer 2 (161)

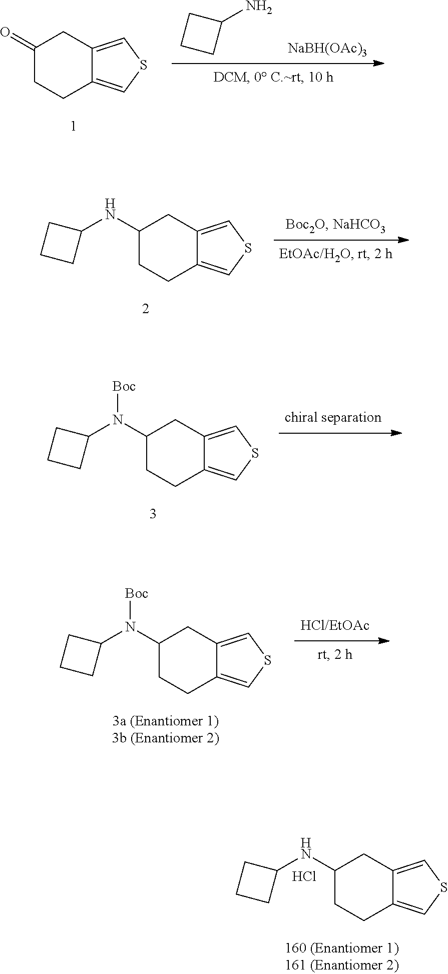

### Procedure for preparation of N-cyclobutyl-4,5,6,7-tetrahydro-2-benzothiophen-5-amine (2)

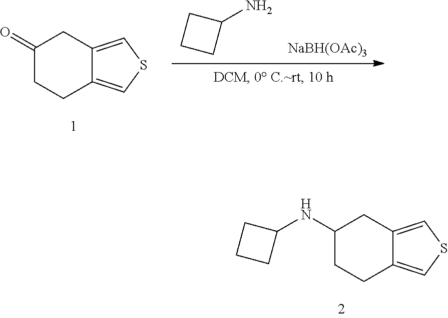

To a solution of 6,7-dihydro-4H-2-benzothiophen-5-one (700 mg, 4.60 mmol, 1 eq) in DCM (10 mL) was added a solution of cyclobutanamine (1) (981.46 mg, 13.80 mmol, 1.18 mL, 3 eq) in DCM (10 mL) at 0° C., then was added NaBH(OAc)₃ (1.95 g, 9.20 mmol, 2 eq) slowly at 0° C. The mixture was stirred at 25° C. for 10 hr. The reaction mixture was diluted with H₂O (30 mL) and extracted with DCM (20 mL*2). The combined organic layers were washed with brine (30 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~100% A/Petroleum ether gradient (A: Ethyl acetate/NH₃·MeOH (7N)=100/1) @40 mL/min) to give N-cyclobutyl-4,5,6,7-tetrahydro-2-benzothiophen-5-amine (2) (500 mg, 2.41 mmol, 52.43% yield) as a colorless oil.

### Procedure for preparation of tert-butyl N-cyclobutyl-N-(4,5,6,7-tetrahydro-2-benzothiophen-5-yl)carbamate enantiomer 1 (3a) and enantiomer 2 (3b)

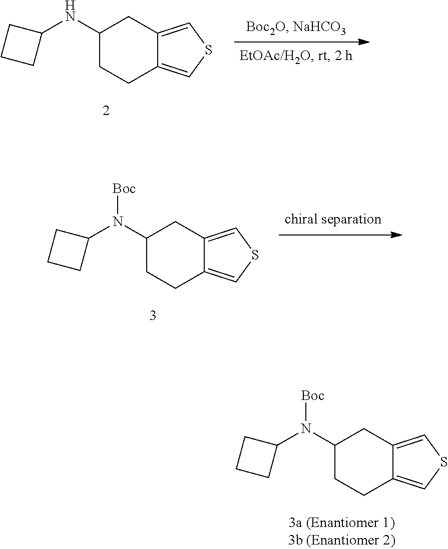 [SMILES: c1scc2c1CCC(NC1CCC1)C2 ; CC(C)(C)OC(=O)N(C1CCC1)C1CCc2cscc2C1 ; CC(C)(C)OC(=O)N(C1CCC1)C1CCc2cscc2C1]

To a solution of N-cyclobutyl-4,5,6,7-tetrahydro-2-benzothiophen-5-amine (2) (500 mg, 2.41 mmol, 1 eq) in EtOAc (5 mL) and H₂O (5 mL) was added NaHCO₃ (405.17 mg, 4.82 mmol, 2 eq) and Boc₂O (1.05 g, 4.82 mmol, 1.11 mL, 2 eq). The mixture was stirred at 25° C. for 2 hr. The reaction mixture was diluted with H₂O 10 mL and extracted with EtOAc (10 mL*2). The combined organic layers were washed with brine (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~5% Ethyl acetate/Petroleum ether gradient @30 mL/min) to give tert-butyl N-cyclobutyl-N-(4,5,6,7-tetrahydro-2-benzothiophen-5-yl) carbamate (3) (700 mg, 2.28 mmol, 94.41% yield) as a colorless oil. This product was separated by SFC (column: DAICEL CHIRALPAK IG 250 mm*30 mm*5 μm; mobile phase: [0.1% NH₃H₂O MeOH]; B %: 20%-20%, 2.8 min) to afford the 1^{st} enantiomer 3a (RT=0.819 min, 255 mg, 829.40 mol) and the 2^{nd} enantiomer 3b (RT=0.922 min, 230 mg, 748.09 mol) both as yellow oils.

### Procedure for preparation of N-cyclobutyl-4,5,6,7-tetrahydro-2-benzothiophen-5-amine hydrochloride enantiomer 1 (160)

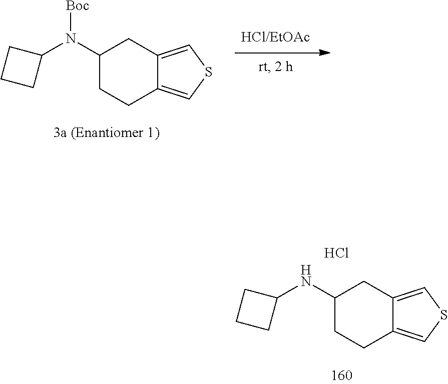 [SMILES: CC(C)(C)OC(=O)N(C1CCC1)C1CCc2cscc2C1 ; Cl ; c1scc2c1CCC(NC1CCC1)C2]

To a solution of tert-butyl N-cyclobutyl-N-(4,5,6,7-tetrahydro-2-benzothiophen-5-yl) carbamate enantiomer 1 (3a) (255 mg, 829 μmol, 1 eq) in EtOAc (2 mL) was added HCl/EtOAc (4 M, 207 μL, 1.00 eq). The mixture was stirred at 25° C. for 2 hr. The reaction mixture was filtered and the cake was washed with EtOAc (10 mL) to afford N-cyclobutyl-4,5,6,7-tetrahydro-2-benzothiophen-5-amine hydrochloride enantiomer 1 (160) (199.46 mg, 817.33 μmol, 98.54% yield) as a white solid. ¹H NMR (400 MHz, METHANOL-d₄) δ 7.11-7.07 (m, 1H), 7.05-7.01 (m, 1H), 4.00 (quin, J=8.2 Hz, 1H), 3.46 (ddt, J=2.8, 5.2, 10.4 Hz, 1H), 3.30-3.23 (m, 1H), 2.98 (td, J=4.8, 16.8 Hz, 1H), 2.88-2.71 (m, 2H), 2.49-2.34 (m, 2H), 2.34-2.19 (m, 3H), 2.02-1.92 (m, 2H), 1.92-1.76 (m, 1H) LCMS: m/z [M+H]⁺=208.1.

### Procedure for preparation of N-cyclobutyl-4,5,6,7-tetrahydro-2-benzothiophen-5-amine hydrochloride enantiomer 2 (161)

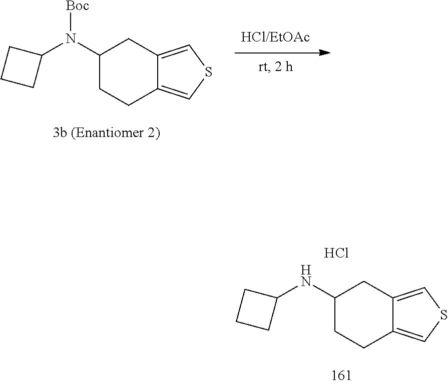 [SMILES: CC(C)(C)OC(=O)N(C1CCC1)C1CCc2cscc2C1 ; Cl ; c1scc2c1CCC(NC1CCC1)C2]

To a solution of tert-butyl N-cyclobutyl-N-(4,5,6,7-tetrahydro-2-benzothiophen-5-yl) carbamate enantiomer 2 (3b) (230 mg, 748 μmol, 1 eq) in EtOAc (2 mL) was added HCl/EtOAc (4 M, 187.02 μL, 1.00 eq). The mixture was stirred at 25° C. for 2 hr. The reaction mixture was filtered and the cake was washed with EtOAc (10 mL) to afford N-cyclobutyl-4,5,6,7-tetrahydro-2-benzothiophen-5-amine hydrochloride enantiomer 2 (161) (174.15 mg, 714.33 μmol, 95.49% yield) as a white solid. ¹H NMR (400 MHz, METHANOL-d₄) δ 7.10 (d, J=2.9 Hz, 1H), 7.05-7.02 (m, 1H), 3.99 (quin, J=8.2 Hz, 1H), 3.46 (ddt, J=2.8, 5.2, 10.4 Hz, 1H), 3.30-3.23 (m, 1H), 2.98 (td, J=4.9, 16.8 Hz, 1H), 2.89-2.69 (m, 2H), 2.47-2.33 (m, 2H), 2.31-2.18 (m, 3H), 2.05-1.91 (m, 2H), 1.83 (dtd, J=5.6, 10.9, 12.8 Hz, 1H). LCMS: m/z [M+H]⁺=208.0

### Synthetic scheme for preparation of 3-methyl-4,5,6,7-tetrahydro-2-benzothiophen-5-amine hydrochloride enantiomer 1 (162) and enantiomer 2 (163), 1-methyl-4,5,6,7-tetrahydro-2-benzothiophen-5-amine hydrochloride enantiomer 1 (164) and enantiomer 2 (165)

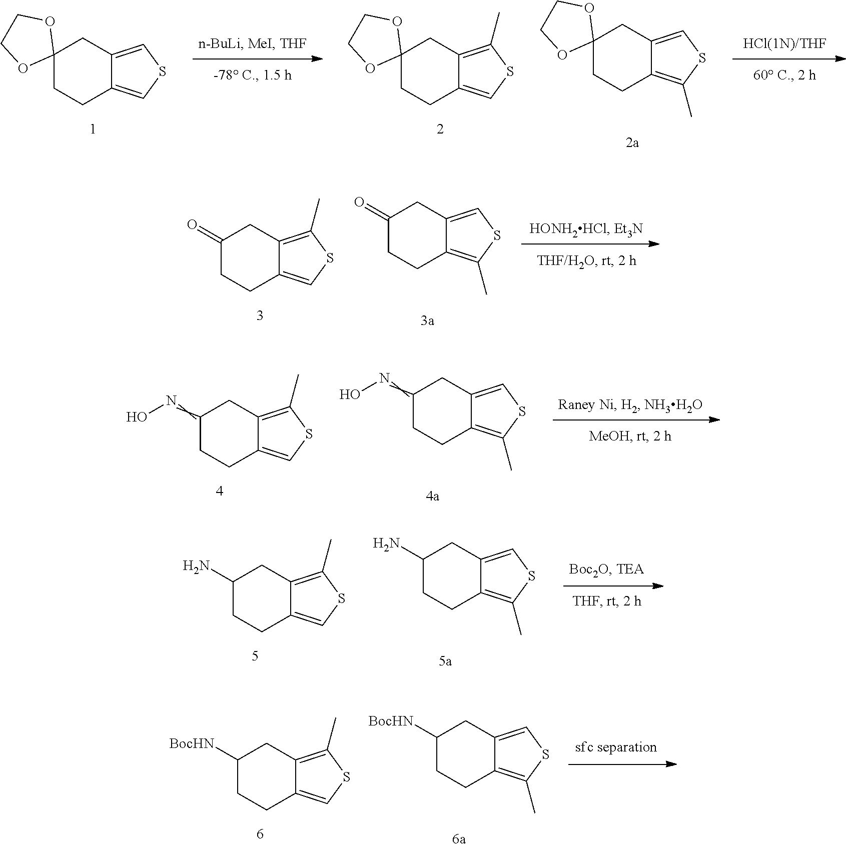

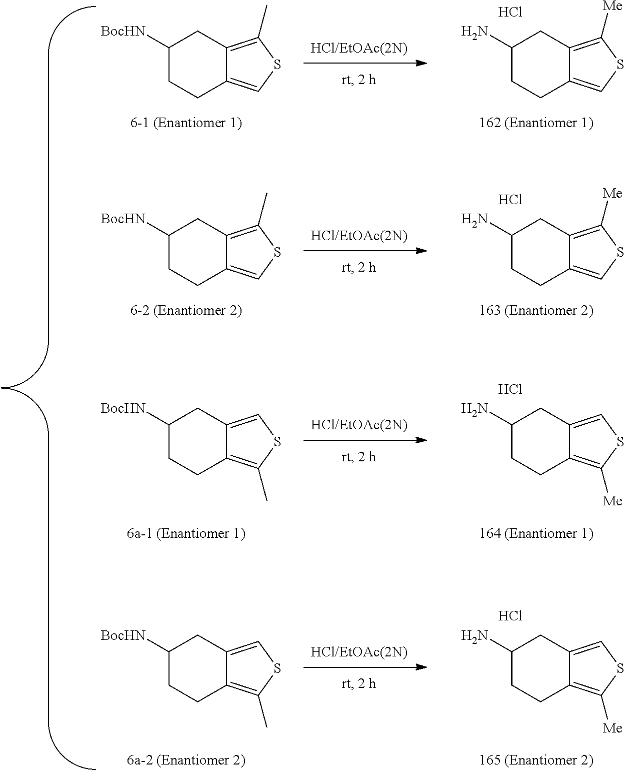

### Procedure for preparation of 3-methyl-6,7-dihydro-4H-spiro[benzo[c]thiophene-5,2′-[1,3]dioxolane](2) and 1-methyl-6,7-dihydro-4H-spiro[benzo[c]thiophene-5,2′-[1,3]dioxolane](2a)

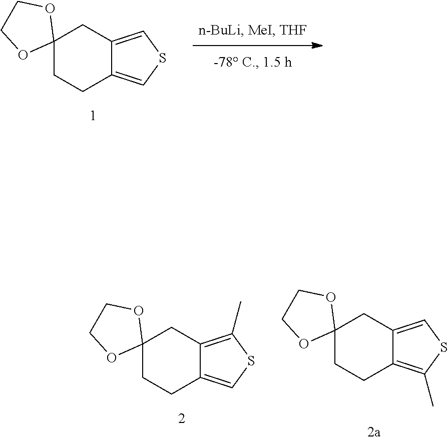

To a solution of spiro[1,3-dioxolane-2,6′-5,7-dihydro-4H-2-benzothiophene](1) (2.8 g, 14.3 mmol, 1 eq) in THF (30 mL) was added dropwise n-BuLi (2.5 M, 6.85 mL, 1.2 eq) at −78° C. After addition, the mixture was stirred at −78° C. for 0.5 hr, and then MeI (2.43 g, 17.1 mmol, 1.07 mL, 1.2 eq) was added dropwise at −78° C. The resulting mixture was stirred at −78° C. for 1 hr. The reaction mixture was poured into H₂O (20 mL) and extracted with EtOAc (20 mL*2). The combined organic phase was washed with brine (10 mL), dried over anhydrous sodium sulfate, filtered and concentrated under vacuum to give a residue. The residue was purified by flash silica gel chromatography (ISCO®; 20 g SepaFlash® Silica Flash Column, eluent 0~10% Ethyl acetate/Petroleum ether gradient @40 mL/min) to afford a mixture of 3-methyl-6,7-dihydro-4H-spiro[benzo[c]thiophene-5,2′-[1,3]dioxolane](2) and 1-methyl-6,7-dihydro-4H-spiro[benzo[c]thiophene-5,2′-[1,3]dioxolane](2a) (2.4 g, 11.4 mmol) as a yellow oil. ¹H NMR (400 MHz, CHLOROFORM-d) δ=6.69-6.65 (m, 1H), 4.07-4.01 (m, 4H), 2.90-2.83 (m, 2H), 2.79-2.70 (m, 2H), 2.33-2.29 (m, 3H), 2.01-1.86 (m, 2H). LCMS: m/z [M+H]⁺=211.0.

### Procedure for preparation of 3-methyl-6,7-dihydro-4H-2-benzothiophen-5-one (3) and 1-methyl-6,7-dihydro-4H-2-benzothiophen-5-one (3a)

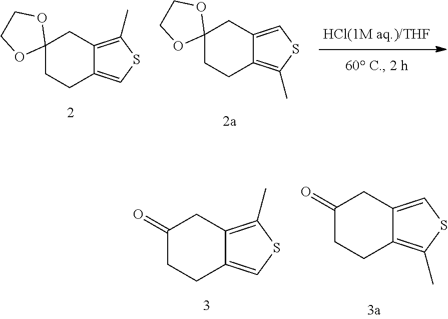

2.4 g (11.4 mmol, 1 eq) mixture of 3-methyl-6,7-dihydro-4H-spiro[benzo[c]thiophene-5,2′-[1,3]dioxolane](2) and 1-methyl-6,7-dihydro-4H-spiro[benzo[c]thiophene-5,2′-[1,3]dioxolane](2a) was added THF (30 mL) and HCl (1 M, 30 mL, 2.63 eq). The mixture was stirred at 60° C. for 2 hr. The reaction mixture was poured into H₂O (20 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 20 g SepaFlash® Silica Flash Column, eluent 0~10% Ethyl acetate/Petroleum ether gradient @40 mL/min) to afford a mixture of 3-methyl-6, 7-dihydro-4H-2-benzothiophen-5-one (3) and 1-methyl-6,7-dihydro-4H-2-benzothiophen-5-one (3a) (1.8 g, 10.8 mmol) as a yellow oil. ¹H NMR (400 MHz, CHLOROFORM-d) δ=6.78 (s, 1H), 3.43 (s, 2H), 2.97 (t, J=6.7 Hz, 2H), 2.63-2.54 (m, 4H), 2.33 (s, 3H).

### Procedure for preparation of 3-methyl-6,7-dihydro-4H-2-benzothiophen-5-one oxime (4) and 1-methyl-6,7-dihydro-4H-2-benzothiophen-5-one oxime (4a)

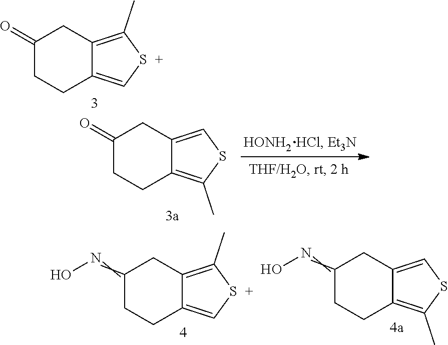

To a solution of 1.8 g (10.8 mmol, 1 eq) of 3-methyl-6,7-dihydro-4H-2-benzothiophen-5-one (3) and 1-methyl-6,7-dihydro-4H-2-benzothiophen-5-one (3a) in THF (20 mL) was added NH₂OH·HCl (1.50 g, 21.7 mmol, 2 eq) and TEA (3.29 g, 32.5 mmol, 4.52 mL, 3 eq). The mixture was stirred at 25° C. for 2 hr. The reaction mixture was added water and extracted with EtOAc (20 mL*2). The combined organic layers were washed with sat.NaCl (20 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~30% Ethyl acetate/Petroleum ether gradient @40 mL/min) to afford mixture of 3-methyl-6,7-dihydro-4H-2-benzothiophen-5-one oxime (4) and 1-methyl-6,7-dihydro-4H-2-benzothiophen-5-one oxime (4a) (1.65 g, 9.10 mmol, 84.07% yield) as a yellow solid.

### Procedure for preparation of 3-methyl-4,5,6,7-tetrahydro-2-benzothiophen-5-amine (5) and 1-methyl-4,5,6,7-tetrahydro-2-benzothiophen-5-amine (5a)

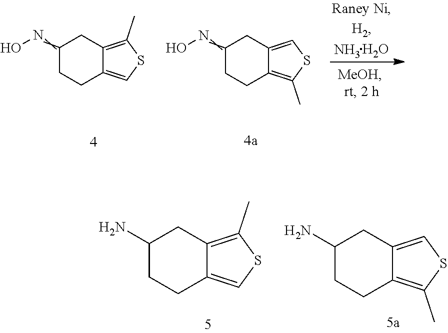

To a solution of 1.65 g (9.10 mmol, 1 eq) of 3-methyl-6,7-dihydro-4H-2-benzothiophen-5-one oxime (4) and 1-methyl-6, 7-dihydro-4H-2-benzothiophen-5-one oxime (4a) in MeOH (30 mL) was added Raney-Ni (800 mg, 9.10 mmol, 1 eq) and NH₃·H₂O (911.51 mg, 18.21 mmol, 1.00 mL, 2 eq) under N₂ atmosphere. The suspension was degassed and purged with H₂ for 3 times. The mixture was stirred under H₂ (15 psi) at 25° C. for 2 hr. Reaction mixture was diluted with MeOH. Mixture was filtered and filtrate was concentrated under vacuum to afford a mixture of 3-methyl-4,5,6,7-tetrahydro-2-benzothiophen-5-amine (5) and 1-methyl-4,5,6,7-tetrahydro-2-benzothiophen-5-amine (5a) (2.1 g, crude) as a yellow oil. LCMS: m/z [M+H]⁺=168.1.

### Procedure for preparation of tert-butyl N-(3-methyl-4,5,6,7-tetrahydro-2-benzothiophen-5-yl)carbamate (6) and tert-butyl N-(1-methyl-4,5,6,7-tetrahydro-2-benzothiophen-5-yl)carbamate (6a)

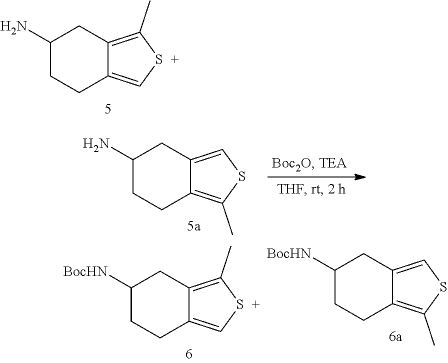

To a solution of a mixture (2.1 g, 12.6 mmol, 1 eq) of 3-methyl-4,5,6,7-tetrahydro-2-benzothiophen-5-amine (5) and 1-methyl-4,5,6,7-tetrahydro-2-benzothiophen-5-amine (5a) in THF (20 mL) was added TEA (2.54 g, 25.1 mmol, 3.49 mL, 2 eq) and (Boc)₂O (5.48 g, 25.1 mmol, 5.77 mL, 2 eq). The mixture was stirred at 25° C. for 2 hr. The reaction mixture was concentrated under reduced pressure to give a residue. The residue was purified by flash silica gel chromatography (ISCO®; 12 g SepaFlash® Silica Flash Column, eluent 0~5% Ethyl acetate/Petroleum ether gradient @40 mL/min). The residue was further purified by prep-HPLC (column: Waters Xbridge C18 150*50 mm*10 μm; mobile phase: [water (NH₄HCO₃)-ACN]; B %: 46%-76%, 10 min) to give 800 mg mixture of tert-butyl N-(3-methyl-4,5,6,7-tetrahydro-2-benzothiophen-5-yl)carbamate (6) and tert-butyl N-(1-methyl-4, 5, 6, 7-tetrahydro-2-benzothiophen-5-yl) carbamate (6a) as a yellow oil.

### Procedure for preparation of tert-butyl N-(3-methyl-4,5,6,7-tetrahydro-2-benzothiophen-5-yl)carbamate enantiomer 1 (6-1) and enantiomer 2 (6-2) and tert-butyl N-(1-methyl-4,5,6,7-tetrahydro-2-benzothiophen-5-yl)carbamate enantiomer 1 (6a-1) and enantiomer 2 (6a-2)

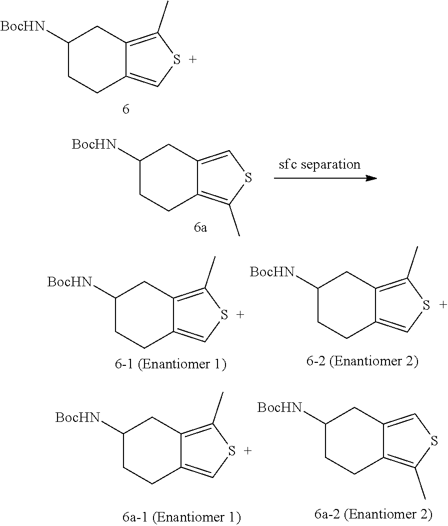

800 mg mixture of tert-butyl N-(3-methyl-4,5,6,7-tetrahydro-2-benzothiophen-5-yl)carbamate (6) and tert-butyl N-(1-methyl-4,5,6,7-tetrahydro-2-benzothiophen-5-yl)carbamate (6a) was separated by SFC (column: DAICEL CHIRALPAK IG 250 mm*30 mm*5 μm; mobile phase: [0.1% NH₃H₂O MEOH]; B %: 15%-15%, 4.2 min) to afford tert-butyl N-(3-methyl-4,5,6,7-tetrahydro-2-benzothiophen-5-yl)carbamate enantiomer 1, (6-1) (RT=0.909 min 210 mg, 785.38 mol) and tert-butyl N-(1-methyl-4,5,6,7-tetrahydro-2-benzothiophen-5-yl)carbamate enantiomer 2 (6a-2)) (RT=1.077 110 mg, 411.39 mol) both as off-white solids and a mixture of tert-butyl N-(3-methyl-4,5,6,7-tetrahydro-2-benzothiophen-5-yl)carbamate enantiomer 2 (6-2)) and tert-butyl N-(1-methyl-4,5,6,7-tetrahydro-2-benzothiophen-5-yl)carbamate enantiomer 1 (6a-1)).

The mixture of compounds 6-2, 6a-1 was separated by SFC (column: DAICEL CHIRALPAK IC (250 mm*30 mm, 5 μm); mobile phase: [0.1% NH₃H₂O IPA]; B %: 20%-20%, 3.2 min) to afford tert-butyl N-(3-methyl-4,5,6,7-tetrahydro-2-benzothiophen-5-yl)carbamate enantiomer 2 (6-2) (RT=0.978 min, 200 mg, 747.98 mol) and tert-butyl N-(1-methyl-4,5,6,7-tetrahydro-2-benzothiophen-5-yl)carbamate enantiomer 1 (6a-1) (RT=1.163 min, 90 mg, 336.59 mol). tert-Butyl N-(3-methyl-4,5,6,7-tetrahydro-2-benzothiophen-5-yl)carbamate enantiomer 1 (6-1): ¹H NMR (400 MHz, METHANOL-d₄) δ=6.65 (s, 1H), 3.78-3.63 (m, 1H), 2.91 (dd, J=5.0, 15.9 Hz, 1H), 2.86-2.77 (m, 1H), 2.72-2.60 (m, 1H), 2.34-2.24 (m, 4H), 2.01-1.92 (m, 1H), 1.65-1.53 (m, 1H), 1.46 (s, 9H). tert-Butyl N-(1-methyl-4,5,6,7-tetrahydro-2-benzothiophen-5-yl)carbamate enantiomer 2 (6a-2): ¹H NMR (400 MHz, METHANOL-d₄) δ=6.66 (s, 1H), 3.73-3.61 (m, 1H), 2.96 (dd, J=5.0, 15.6 Hz, 1H), 2.73 (td, J=4.7, 16.8 Hz, 1H), 2.61-2.39 (m, 2H), 2.28 (s, 3H), 2.08-1.96 (m, 1H), 1.72-1.58 (m, 1H), 1.45 (s, 9H). LCMS: m/z [M+H-Boc]⁺=212.2.

### Procedure for preparation of 3-methyl-4,5,6,7-tetrahydro-2-benzothiophen-5-amine hydrochloride enantiomer 1 (162)

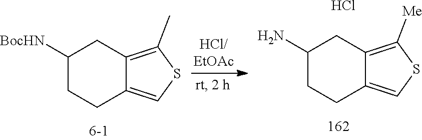 [SMILES: Cl ; Cc1scc2c1CC(NC(=O)OC(C)(C)C)CC2 ; Cc1scc2c1CC(N)CC2]

To a solution of tert-butyl N-(3-methyl-4,5,6,7-tetrahydro-2-benzothiophen-5-yl) carbamate enantiomer 1 (6-1) (210 mg, 785 μmol, 1 eq) in EtOAc (2 mL) was added HCl/EtOAc (4 M, 5 mL, 25.5 eq). The mixture was stirred at 25° C. for 2 hr. The reaction mixture was filtered and the cake was washed with EtOAc (10 mL) to afford 3-methyl-4,5,6,7-tetrahydro-2-benzothiophen-5-amine hydrochloride enantiomer 1 (162) (148.70 mg, 725.72 μmol, 92.40% yield) as a white solid. ¹H NMR (400 MHz, METHANOL-d4) δ=6.79 (s, 1H), 3.56-3.48 (m, 1H), 3.11 (dd, J=5.4, 15.5 Hz, 1H), 2.99-2.88 (m, 1H), 2.83-2.70 (m, 1H), 2.53 (dd, J=9.9, 15.4 Hz, 1H), 2.34 (s, 3H), 2.21-2.11 (m, 1H), 1.81 (dtd, J=5.4, 11.1, 12.7 Hz, 1H). LCMS: m/z [M+H]⁺=168.1

### Procedure for preparation of 3-methyl-4,5,6,7-tetrahydro-2-benzothiophen-5-amine hydrochloride enantiomer 2 (163)

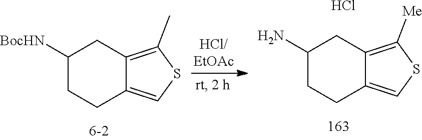 [SMILES: Cl ; Cc1scc2c1CC(NC(=O)OC(C)(C)C)CC2 ; Cc1scc2c1CC(N)CC2]

To a solution of tert-butyl N-(3-methyl-4,5,6,7-tetrahydro-2-benzothiophen-5-yl) carbamate enantiomer 2 (6-2) (200.00 mg, 748 μmol, 1 eq) in EtOAc (2 mL) was added HCl/EtOAc (4 M, 5 mL, 26.7 eq). The mixture was stirred at 25° C. for 2 hr. The reaction mixture was filtered and the cake was washed with EtOAc (10 mL) to afford 3-methyl-4,5,6,7-tetrahydro-2-benzothiophen-5-amine hydrochloride enantiomer 2 (163) (119.63 mg, 580.38 μmol, 77.59% yield) as a white solid. ¹H NMR (400 MHz, METHANOL-d₄) δ=6.77 (s, 1H), 3.51 (ddt, J=3.1, 5.5, 10.4 Hz, 1H), 3.08 (dd, J=5.4, 15.5 Hz, 1H), 2.97-2.87 (m, 1H), 2.81-2.69 (m, 1H), 2.51 (dd, J=9.9, 15.8 Hz, 1H), 2.32 (s, 3H), 2.19-2.09 (m, 1H), 1.85-1.73 (m, 1H). LCMS: m/z [M+H]⁺=168.1

### Procedure for preparation of 1-methyl-4,5,6,7-tetrahydro-2-benzothiophen-5-amine hydrochloride enantiomer 1 (164)

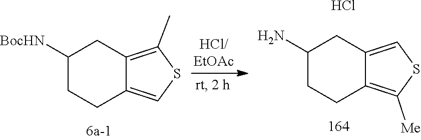 [SMILES: Cl ; Cc1scc2c1CC(NC(=O)OC(C)(C)C)CC2 ; Cc1scc2c1CCC(N)C2]

To a solution of tert-butyl N-(1-methyl-4,5,6,7-tetrahydro-2-benzothiophen-5-yl) carbamate enantiomer 1 (6a-1) (90 mg, 336 μmol, 1 eq) in EtOAc (2 mL) was added HCl/EtOAc (4 M, 5 mL, 59.4 eq). The mixture was stirred at 25° C. for 2 hr. The reaction mixture was filtered and the cake was washed with EtOAc (10 mL) to afford 1-methyl-4, 5, 6, 7-tetrahydro-2-benzothiophen-5-amine hydrochloride enantiomer 1 (164) (52.58 mg, 309.34 μmol, 91.90% yield) as an off-white solid. ¹H NMR (400 MHz, METHANOL-d₄) 6=6.82 (s, 1H), 3.53-3.42 (m, 1H), 3.20-3.11 (m, 1H), 2.89-2.78 (m, 1H), 2.74-2.56 (m, 2H), 2.31 (s, 3H), 2.25-2.16 (m, 1H), 1.91-1.77 (m, 1H). LCMS: m/z [M+H]⁺=168.1

### Procedure for preparation of 1-methyl-4,5,6,7-tetrahydro-2-benzothiophen-5-amine hydrochloride enantiomer 2 (165)

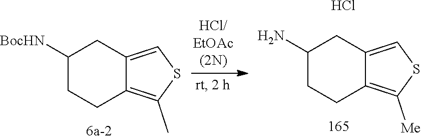 [SMILES: Cl ; Cc1scc2c1CCC(NC(=O)OC(C)(C)C)C2 ; Cc1scc2c1CCC(N)C2]

To a solution of tert-butyl N-(1-methyl-4,5,6,7-tetrahydro-2-benzothiophen-5-yl) carbamate enantiomer 2 (6a-2) (110.00 mg, 411 μmol, 1 eq) in EtOAc (2 mL) was added HCl/EtOAc (4 M, 5 mL, 48.6 eq). The mixture was stirred at 25° C. for 2 hr. The reaction mixture was filtered and the cake was washed with EtOAc (10 mL) to afford 1-methyl-4, 5, 6, 7-tetrahydro-2-benzothiophen-5-amine hydrochloride enantiomer 2 (165) (68.41 mg, 335.25 μmol, 81.49% yield) as an off-white solid. ¹H NMR (400 MHz, METHANOL-d₄) 6=6.82 (s, 1H), 3.53-3.42 (m, 1H), 3.15 (br dd, J=4.5, 15.7 Hz, 1H), 2.88-2.79 (m, 1H), 2.74-2.56 (m, 2H), 2.31 (s, 3H), 2.25-2.16 (m, 1H), 1.91-1.77 (m, 1H). LCMS: m/z [M+H]⁺=168.1

### Synthetic scheme for preparation of 4,5,6,7-tetrahydro-2-benzothiophen-5-amine hydrochloride enantiomer 1 (166) and enantiomer 2 (167)

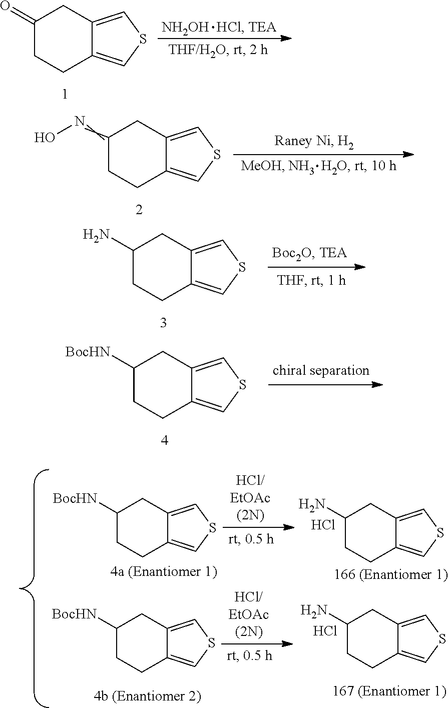

### Procedure for preparation of 6,7-dihydro-4H-2-benzothiophen-5-one oxime (2)

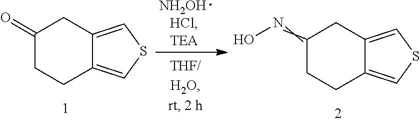

To a mixture of 6,7-dihydro-4H-2-benzothiophen-5-one (1) (3.0 g, 19.71 mmol, 1 eq), NH₂OH·HCl (2.74 g, 39.42 mmol, 2 eq) in THF (10 mL) and H₂O (10 mL) was added TEA (3.99 g, 39.42 mmol, 5.49 mL, 2 eq), and the resulting mixture was stirred at 25° C. for 2 hr. The reaction mixture was diluted with H₂O (30 mL) and extracted with EtOAc (30 mL*2). The combined organic layers were washed with brine (30 mL), dried over anhydrous sodium sulfate, filtered, and concentrated under reduced pressure. The residue was purified by flash silica gel chromatography (ISCO®; 40 g SepaFlash® Silica Flash Column, eluent 0~15% Ethyl acetate/Petroleum ether) to give 6,7-dihydro-4H-2-benzothiophen-5-one oxime (2) (3 g, 17.04 mmol, 86.47% yield) as a white solid. LCMS: m/z [M+H]⁺=168.1.

### Procedure for preparation of 4,5,6,7-tetrahydro-2-benzothiophen-5-amine (3)

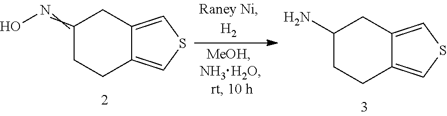

To a suspension of Raney-Ni (1.46 g) in MeOH (50 mL) was added a solution of 6,7-dihydro-4H-2-benzothiophen-5-one oxime (2) (3.00 g, 17.04 mmol, 95% purity, 1 eq) in MeOH (10 mL) and conc. aqueous NH₃ (0.5 mL). The suspension was degassed under vacuum and purged with H₂ several times. The mixture was stirred under H₂ (15 psi) at 20° C. for 10 hours. The reaction mixture was filtered and the filtrate was concentrated in vacuo. The residue was purified by flash silica gel chromatography (ISCO®; 40 g SepaFlash® Silica Flash Column, eluent 0~100% (Ethyl acetate/NH₃·MeOH=100/1)/Petroleum ether) to afford 4,5,6,7-tetrahydro-2-benzothiophen-5-amine (3) (900 mg, 5.81 mmol, 34.12% yield) as a brown oil. LCMS: m/z [M+H]⁺=154.0.

### Procedure for preparation of tert-butyl N-(4,5,6,7-tetrahydro-2-benzothiophen-5-yl) carbamate enantiomer 1 (4a) and enantiomer 2 (4b)

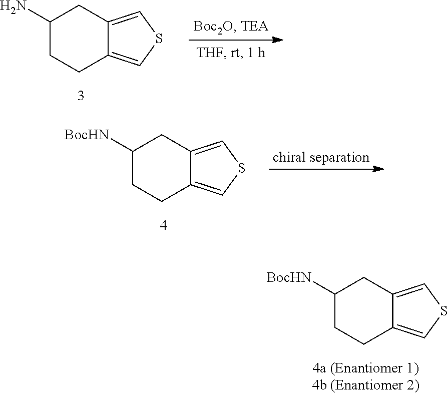 [SMILES: NC1CCc2cscc2C1 ; CC(C)(C)OC(=O)NC1CCc2cscc2C1 ; CC(C)(C)OC(=O)NC1CCc2cscc2C1]

To a solution of 4,5,6,7-tetrahydro-2-benzothiophen-5-amine (3) (850 mg, 5.49 mmol) and Boc₂O (1.80 g, 8.24 mmol, 1.89 mL, 1.5 eq) in THF (20 mL) was added TEA (1.11 g, 10.98 mmol, 1.53 mL, 2 eq), and the resulting mixture was stirred at 25° C. for 1 hr. The reaction mixture was concentrated under reduced pressure. The residue was purified by flash silica gel chromatography (ISCO®; 20 g SepaFlash® Silica Flash Column, eluent 0-10% Ethyl acetate/Petroleum ether) to afford the racemic tert-butyl N-(4,5,6,7-tetrahydro-2-benzothiophen-5-yl)carbamate (4), which was further separated by prep-chiral-SFC (column: DAICEL CHIRALPAK AD-H (250 mm*30 mm, 5 μm); mobile phase: [0.1% NH₃H₂O in MeOH]; B %: 15%-15%, 2.1 min) to enantiomer 1 (4a, 1^{st} peak in SFC) (500 mg, 1.97 mmol)) and enantiomer 2 (4b, 2^{nd} peak in SFC) (480 mg, 1.88 mmol), both as white solids. Enantiomer 1 (4a): ¹H NMR (400 MHz, CHLOROFORM-d) δ 6.94-6.87 (m, 2H), 4.63-4.51 (m, 1H), 4.03-3.92 (m, 1H), 3.09 (dd, J=5.1, 15.8 Hz, 1H), 2.91-2.74 (m, 2H), 2.57 (dd, J=8.0, 15.9 Hz, 1H), 2.08-1.95 (m, 1H), 1.83-1.71 (m, 1H), 1.46 (s, 9H). LCMS: m/z [M+H-tBu]⁺=198.5. Enantiomer 2 (4b): ¹H NMR (400 MHz, CHLOROFORM-d) δ=6.95-6.86 (m, 2H), 4.65-4.51 (m, 1H), 4.01-3.90 (m, 1H), 3.09 (dd, J=5.0, 15.8 Hz, 1H), 2.93-2.74 (m, 2H), 2.57 (dd, J=8.1, 15.8 Hz, 1H), 2.07-1.95 (m, 1H), 1.87-1.72 (m, 1H), 1.46 (s, 9H). LCMS: m/z [M+H-tBu]⁺=198.5.

### Procedure for preparation of 4,5,6,7-tetrahydro-2-benzothiophen-5-amine hydrochloride enantiomer 1 (166)

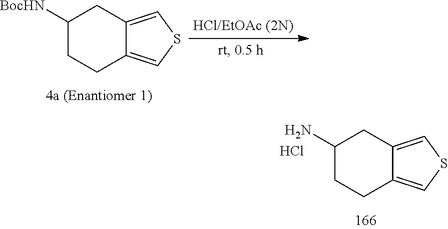 [SMILES: CC(C)(C)OC(=O)NC1CCc2cscc2C1 ; Cl ; NC1CCc2cscc2C1]

To a solution of tert-butyl N-(4,5,6,7-tetrahydro-2-benzothiophen-5-yl) carbamate enantiomer 1 (4a) (500 mg, 1.97 mmol) in EtOAc (4 mL) was added 4 N HCl/EtOAc (4 mL), and the resulting mixture was stirred at 25° C. for 0.5 hr. The precipitate was collected by filtration, purified by trituration with EtOAc (2 mL), and then dried under reduced pressure to give 4,5,6,7-tetrahydro-2-benzothiophen-5-amine hydrochloride enantiomer 1 (166) (370.87 mg, 1.95 mmol, 99.00% yield) as a white solid. ¹H NMR (400 MHz, METHANOL-d4) δ=7.08 (s, 1H), 7.04 (s, 1H), 3.52 (ddt, J=2.9, 5.2, 10.5 Hz, 1H), 3.24 (dd, J=5.1, 15.6 Hz, 1H), 2.99 (td, J=4.8, 16.7 Hz, 1H), 2.86-2.69 (m, 2H), 2.25-2.14 (m, 1H), 1.92-1.79 (m, 1H). LCMS: m/z [M+H]⁺=154.0.

### Procedure for preparation of 4,5,6,7-tetrahydro-2-benzothiophen-5-amine hydrochloride enantiomer 2 (167)

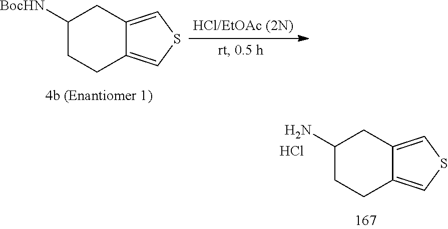 [SMILES: CC(C)(C)OC(=O)NC1CCc2cscc2C1 ; Cl ; NC1CCc2cscc2C1]

To a solution of tert-butyl N-(4,5,6,7-tetrahydro-2-benzothiophen-5-yl)carbamate enantiomer 2 (4b) (480 mg, 1.86 mmol) in EtOAc (4 mL) was added 4 N HCl/EtOAc (4 mL), and the resulting mixture was stirred at 25° C. for 0.5 hr. The precipitate was collected by filtration, purified by trituration with EtOAc (2 mL), and then dried under reduced pressure to give 4,5,6,7-tetrahydro-2-benzothiophen-5-amine hydrochloride enantiomer 2 (167) (345.4 mg, 1.82 mmol, 98.06% yield) as a white solid. ¹H NMR (400 MHz, METHANOL-d4) δ=7.08 (s, 1H), 7.04 (s, 2H), 3.52 (ddt, J=2.9, 5.2, 10.5 Hz, 1H), 3.24 (dd, J=4.6, 15.6 Hz, 1H), 2.99 (td, J=4.7, 16.7 Hz, 1H), 2.88-2.68 (m, 2H), 2.27-2.15 (m, 1H), 1.93-1.77 (m, 1H). LCMS: m/z [M+H]⁺=154.0.

### Synthetic scheme for preparation of N-methyl-5,6-dihydro-4H-cyclohepta[c]thiophen-5-amine hydrochloride (168), and N-methyl-5,6,7,8-tetrahydro-4H-cyclohepta[c]thiophen-5-amine hydrochloride enantiomer 1 (169) and enantiomer 2 (170)

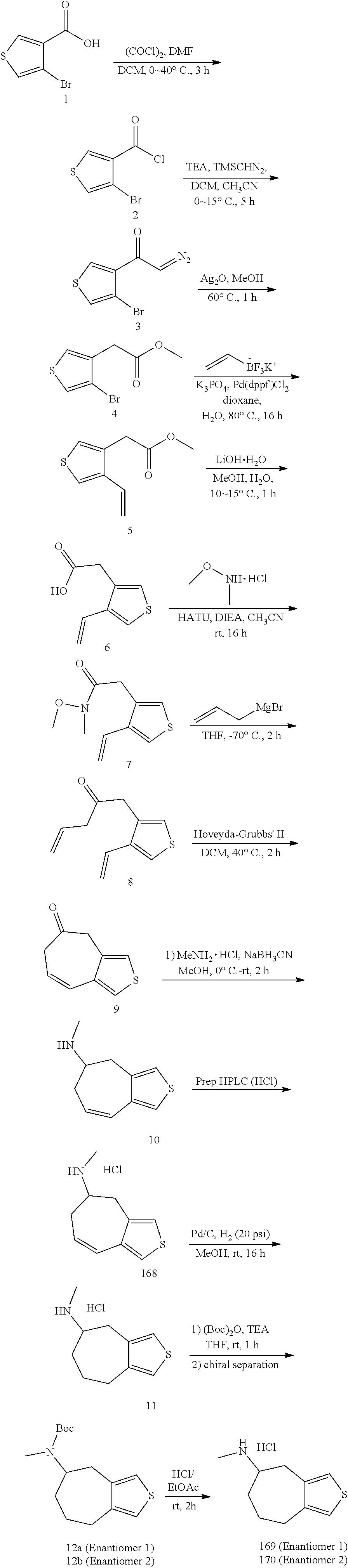

### Procedure for preparation of 4-bromothiophene-3-carbonyl chloride (2)

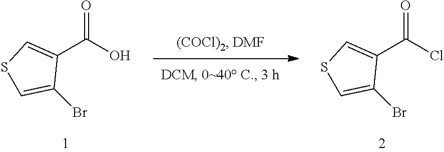 [SMILES: O=C(O)c1cscc1Br ; O=C(Cl)c1cscc1Br]

To the mixture of 4-bromothiophene-3-carboxylic acid (1) (40 g, 193.19 mmol, 1 eq) in DCM (400 mL) and DMF (282 mg, 3.86 mmol, 297 mL, 0.02 eq) was added (COCl)₂ (33.1 g, 261 mmol, 22.8 mL, 1.35 eq) drop-wise at 0-5° C. After addition, the mixture was allowed to warm to 40° C. and stirred for 3 hr. The reaction mixture was concentrated under vacuum to yield 4-bromothiophene-3-carbonyl chloride (2) (44 g, crude) as a light yellow solid.

### Procedure for preparation of 1-(4-bromo-3-thienyl)-2-diazo-ethanone (3)

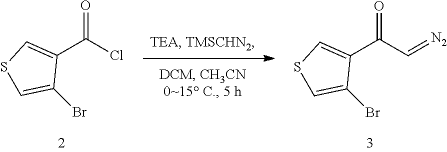

To the mixture of 4-bromothiophene-3-carbonyl chloride (2) (44 g, 195 mmol, 1 eq) in CH₃CN (600 mL) and THF (600 mL) was added TEA (39.49 g, 390.26 mmol, 54.32 mL, 2 eq) and diazomethyl (trimethyl) silane (2 M, 214.64 mL, 2.2 eq) at 0° C. After addition, the mixture was stirred at 10-15° C. for 5 hr. The reaction mixture was concentrated under vacuum to give a residue. The residue was purified by flash silica gel chromatography (ISCO®; 40 g SepaFlash® Silica Flash Column, eluent 0~15% Ethyl acetate/Petroleum ether gradient @40 mL/min) to yield 1-(4-bromo-3-thienyl)-2-diazo-ethanone (3) (40 g, 173.11 mmol, 88.71% yield) as a yellow solid. ¹H NMR (400 MHz, DMSO-d6, 298 K) δ 8.28 (d, J=3.2 Hz, 1H), 7.79 (d, J=3.3 Hz, 1H), 6.64 (br s, 1H)

### Procedure for preparation of methyl 2-(4-bromothiophen-3-yl)acetate (4)

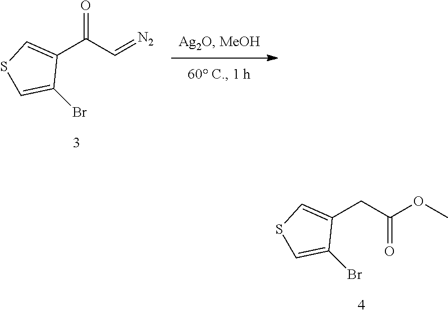

To the solution of 1-(4-bromo-3-thienyl)-2-diazo-ethanone (3) (40 g, 173 mmol, 1 eq) in MeOH (400 mL) was added Ag₂O (40.1 g, 173 mmol, 1 eq). The resulting mixture was stirred at 60° C. for 1 hr. The reaction mixture was filtered and the cake was washed with EtOAc (50 mL). The filtrate was concentrated under vacuum to give a residue which was purified by flash silica gel chromatography (ISCO®; 120 g SepaFlash® Silica Flash Column, eluent 0~10% Ethyl acetate/Petroleum ether gradient @40 mL/min) to yield methyl 2-(4-bromothiophen-3-yl)acetate (4) (19.4 g, 82.52 mmol, 47.67% yield) as a yellow oil. ¹H NMR (400 MHz, CDCl3, 298 K) δ 7.30-7.27 (m, 2H), 3.76 (s, 3H), 3.69 (s, 2H)

### Procedure for preparation of methyl 2-(4-vinylthiophen-3-yl)acetate (5)

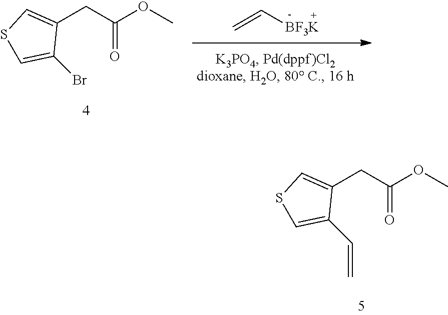

To the mixture of methyl 2-(4-bromothiophen-3-yl)acetate (4) (19.4 g, 82.5 mmol) potassium trifluoro(vinyl)borate (27.6 g, 206 mmol, 2.5 eq), K₃PO₄ (52.6 g, 248 mmol, 3 eq) in dioxane (250 mL) and H₂O (30 mL) was added Pd(dppf) Cl₂ (3.02 g, 4.13 mmol, 0.05 eq). The resulting mixture was purged with N₂ for three times and stirred at 80° C. for 16 hr. The reaction mixture was filtered and the cake was washed with EtOAc (100 mL). The filtrate was concentrated under vacuum to give a residue which was purified by flash silica gel chromatography (ISCO®; 120 g SepaFlash® Silica Flash Column, eluent 5-10% Ethyl acetate/Petroleum ether gradient @40 mL/min) to yield methyl 2-(4-vinylthiophen-3-yl)acetate (5) (10 g, 54.8 mmol, 66.5% yield) as a yellow oil ¹H NMR (400 MHz, Methanol-d4, 298 K) δ 7.43 (d, J=3.1 Hz, 1H), 7.20 (d, J=3.3 Hz, 1H), 6.66 (dd, J=11.1, 17.4 Hz, 1H), 5.59 (dd, J=1.4, 17.5 Hz, 1H), 5.19 (dd, J=1.4, 11.1 Hz, 1H), 3.72-3.63 (m, 5H)

### Procedure for preparation of 2-(4-vinylthiophen-3-yl)acetic acid (6)

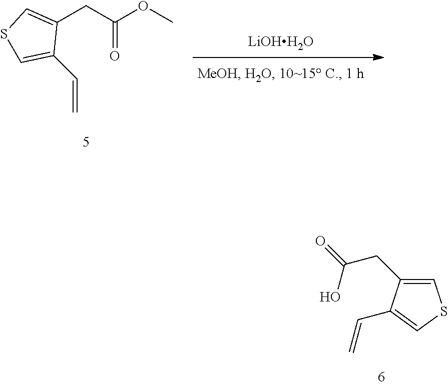

To the mixture of methyl 2-(4-vinylthiophen-3-yl)acetate (5) (10 g, 54.9 mmol, 1 eq) in MeOH (100 mL) and H₂O (100 mL) was added LiOH·H₂O (4.61 g, 110 mmol, 2 eq). The resulting mixture was stirred at 10-15° C. for 1 hr. The reaction mixture was concentrated under vacuum to remove MeOH. The aqueous phase was diluted with water (50 mL), extracted with EtOAc (50 mL). The aqueous phase pH was adjusted with HCl (6 mol/L) to 2-3 and extracted with EtOAc (50 mL*3). The combined organic phase was washed with brine (30 mL), dried over anhydrous sodium sulfate, filtered and concentrated under vacuum to give 2-(4-vinylthiophen-3-yl)acetic acid (6) (8.5 g, 50.5 mmol, 92.1% yield) as a yellow solid ¹H NMR (400 MHz, METHANOL-d₄) δ 7.44 (d, J=3.3 Hz, 1H), 7.21 (d, J=3.3 Hz, 1H), 6.69 (dd, J=11.1, 17.5 Hz, 1H), 5.60 (dd, J=1.4, 17.6 Hz, 1H), 5.20 (dd, J=1.5, 11.1 Hz, 1H), 3.64 (s, 2H).

### Procedure for preparation of N-methoxy-N-methyl-2-(4-vinylthiophen-3-yl)acetamide (7)

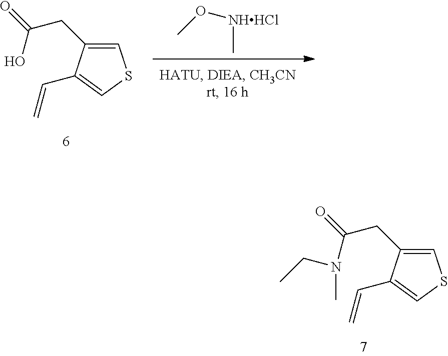

To the mixture of 2-(4-vinylthiophen-3-yl)acetic acid (6) (8.5 g, 50.5 mmol, 1 eq), N-methoxymethanamine hydrochloride (5.91 g, 60.6 mmol, 1.2 eq) and DIEA (19.6 g, 151 mmol, 26.4 mL, 3 eq) in ACN (160 mL) was added HATU (28.8 g, 75.8 mmol, 1.5 eq). After addition, the mixture was stirred at 25° C. for 16 hr. The reaction mixture was diluted with water (30 mL) and concentrated under vacuum to remove solvent. The aqueous phase was extracted with EtOAc (30 mL*3). The combined organic phases were washed with brine (30 mL), dried over anhydrous sodium sulfate, filtered and concentrated under vacuum to give a residue which was purified by flash silica gel chromatography (ISCO®; 120 g SepaFlash® Silica Flash Column, eluent 0~65% Ethyl acetate/Petroleum ether gradient @40 mL/min) to yield N-methoxy-N-methyl-2-(4-vinylthiophen-3-yl)acetamide (7) (9.1 g, 43.07 mmol, 85.24% yield) as a yellow oil. ¹H NMR (400 MHz, CHLOROFORM-d) δ 7.34 (d, J=3.2 Hz, 1H), 7.13 (d, J=3.2 Hz, 1H), 6.70 (dd, J=11.0, 17.5 Hz, 1H), 5.59 (dd, J=1.3, 17.4 Hz, 1H), 5.24 (dd, J=1.3, 10.9 Hz, 1H), 3.78 (s, 2H), 3.63 (s, 3H), 3.21 (s, 3H). LCMS: m/z [M+H]⁺=212.1

### Procedure for preparation of 1-(4-vinylthiophen-3-yl)pent-4-en-2-one (8)

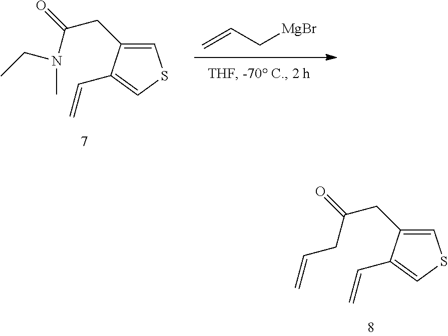

To the mixture of N-methoxy-N-methyl-2-(4-vinylthiophen-3-yl)acetamide (7) (9.1 g, 43.1 mmol, 1 eq) in THF (90 mL) was added allyl(bromo)magnesium (1 M, 45.2 mL, 1.05 eq) at −70° C. under N₂ atmosphere. After addition, the mixture was stirred at −70° C. for 2 hr. The reaction mixture was poured into aqueous sat. NH₄Cl (100 mL) and extracted with EtOAc (50 mL*2). The combined organic phase was washed with brine (100 mL), dried over anhydrous sodium sulfate, filtered and concentrated under vacuum to give a residue. The residue was purified by column chromatography (SiO₂, Petroleum ether/Ethyl acetate=100/0 to 20/1) to yield 1-(4-vinylthiophen-3-yl)pent-4-en-2-one (8) (7 g, 36.4 mmol, 84.5% yield) as a yellow oil. ¹H NMR (400 MHz, CHLOROFORM-d) δ 7.36 (d, J=3.1 Hz, 1H), 7.10 (d, J=2.8 Hz, 1H), 6.56 (dd, J=10.9, 17.4 Hz, 1H), 5.99-5.83 (m, 1H), 5.58 (d, J=17.4 Hz, 1H), 5.30-5.06 (m, 3H), 3.74 (s, 2H), 3.19 (d, J=7.0 Hz, 2H)

### Procedure for preparation of 4,6-dihydrocyclohepta[c]thiophen-5-one (9)

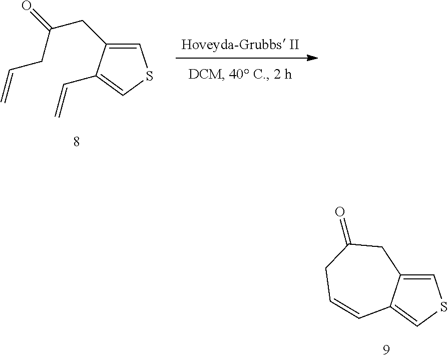

To the solution of 1-(4-vinylthiophen-3-yl)pent-4-en-2-one (8) (7 g, 36.4 mmol, 1 eq) in DCM (300 mL) was added [1,3-bis(2,4,6-trimethylphenyl)imidazolidin-2-ylidene]-dichloro-[(2-isopropoxyphenyl)methylene]ruthenium (1.14 g, 1.82 mmol, 0.05 eq). The resulting mixture was stirred at 40° C. for 2 hr. The reaction mixture was filtered and the cake was washed with EtOAc (20 mL). The reaction mixture was concentrated under vacuum to give a residue. The residue was purified by flash silica gel chromatography (ISCO®; 40 g SepaFlash® Silica Flash Column, eluent 0~6% Ethyl acetate/Petroleum ether gradient @40 mL/min) to yield 4,6-dihydrocyclohepta[c]thiophen-5-one (9) (3 g, 18.3 mmol, 50.2% yield) as a yellow oil. ¹H NMR (400 MHz, CHLOROFORM-d) δ 7.19 (d, J=3.1 Hz, 1H), 7.06 (dd, J=0.8, 3.1 Hz, 1H), 6.77 (d, J=11.0 Hz, 1H), 5.86-5.74 (m, 1H), 3.72 (s, 2H), 3.18 (dd, J=1.1, 5.8 Hz, 2H).

### Procedure for preparation of N-methyl-5,6-dihydro-4H-cyclohepta[c]thiophen-5-amine hydrolchloride (168)

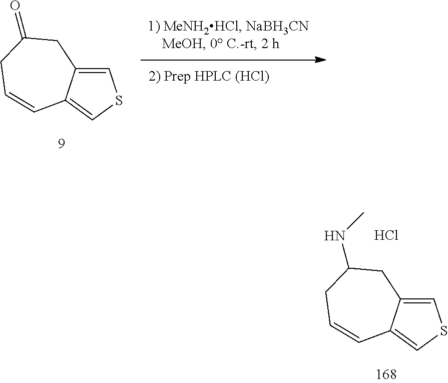

To a mixture of 4,6-dihydrocyclohepta[c]thiophen-5-one (9) (3 g, 18.27 mmol, 1 eq) and methanamine hydrochloride (4.93 g, 73.1 mmol, 4 eq) in MeOH (70 mL) was added NaBH₃CN (6.89 g, 110 mmol, 6 eq) at 0° C. The resulting mixture was stirred at 25° C. for 2 hr. The reaction mixture was diluted with water (10 mL) and concentrated under vacuum to remove the organic solvent. The aqueous phase was extracted with EtOAc (30 mL*3). The combined organic phases were washed with brine (30 mL), dried over anhydrous sodium sulfate, filtered and concentrated under vacuum to give a residue. The residue was purified by flash silica gel chromatography (ISCO®; 40 g SepaFlash® Silica Flash Column, eluent 0-100% Ethyl acetate/Petroleum ether gradient @40 mL/min). Compound N-methyl-5,6-dihydro-4H-cyclohepta[c]thiophen-5-amine (168) (3 g, 16.73 mmol, 91.60% yield) was obtained as a yellow oil.

The product (100 mg) was further purified by prep-HPLC (column: YMC Triart C18 150*25 mm*5 μm; mobile phase: [water (HCl)-ACN]; B %: 8%-38%, 10 min) to yield N-methyl-5, 6-dihydro-4H-cyclohepta[c]thiophen-5-amine hydrochloride (168) (69.07 mg, 317.27 mol) as a white solid. ¹H NMR (400 MHz, METHANOL-d₄) δ 7.31 (d, J=3.1 Hz, 1H), 7.23 (d, J=2.6 Hz, 1H), 6.59 (br d, J=12.0 Hz, 1H), 5.60 (td, J=4.9, 12.2 Hz, 1H), 3.64-3.53 (m, 1H), 3.29-3.18 (m, 2H), 2.93-2.79 (m, 1H), 2.75-2.63 (m, 4H). LCMS: m/z [M+H]⁺=180.0

### Procedure for preparation of N-methyl-5,6,7,8-tetrahydro-4H-cyclohepta[c]thiophen-5-amine hydrochloride (11)

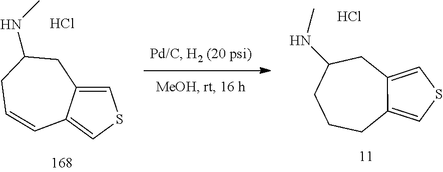

To a solution of N-methyl-5,6-dihydro-4H-cyclohepta[c]thiophen-5-amine hydrochloride (168) (3 g, 16.7 mmol, 1 eq) in MeOH (20 mL) was added Pd/C (3 g, 10% Pd) under N₂ atmosphere. The suspension was degassed and purged with H₂ for 3 times. The mixture was stirred under H₂ (20 psi) at 25° C. for 16 hr. The reaction mixture was filtered and the filtered cake was washed with MeOH (20 mL). The filtrate was concentrated under vacuum to yield N-methyl-5,6,7,8-tetrahydro-4H-cyclohepta[c]thiophen-5-amine hydrochloride (11) (2.4 g, 13.2 mmol, 79.1% yield) as a yellow gum. LCMS: m/z [M+H]⁺=182.4

### Procedure for preparation of tert-butyl N-methyl-N-(5,6,7,8-tetrahydro-4H-cyclohepta[c]thiophen-5-yl)carbamate enantiomer 1 (12a) and enantiomer 2 (12 b)

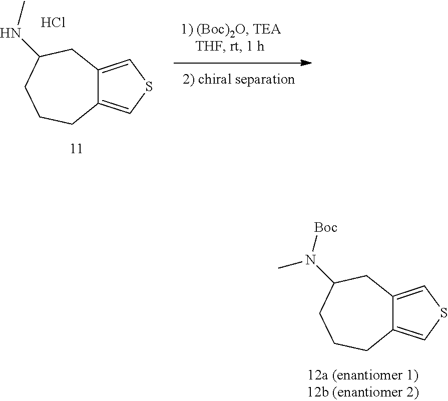 [SMILES: CNC1CCCc2cscc2C1 ; Cl ; CN(C(=O)OC(C)(C)C)C1CCCc2cscc2C1]

To a solution of N-methyl-5, 6, 7, 8-tetrahydro-4H-cyclohepta[c]thiophen-5-amine hydrochloride (11) (800 mg, 4.41 mmol, 1 eq) in THF (10 mL) was added TEA (893 mg, 8.83 mmol, 1.23 mL, 2 eq) and (Boc)₂O (1.93 g, 8.83 mmol, 2.03 mL, 2 eq). The mixture was stirred at 25° C. for 1 hr. The reaction mixture was concentrated under reduced pressure to give a residue. The residue was purified by flash silica gel chromatography (ISCO®; 4 g SepaFlash® Silica Flash Column, eluent 0~5% Ethyl acetate/Petroleum ether gradient @40 mL/min). The residue was separated by SFC (column: DAICEL CHIRALCEL OJ-H (250 mm*30 mm, 5 μm); mobile phase: [0.1% NH₃H₂O IPA]; B %: 15%-15%, 2.8 min) to yield tert-butyl N-methyl-N-(5,6,7,8-tetrahydro-4H-cyclohepta[c]thiophen-5-yl)carbamate enantiomer 1 (12a) (330 mg, 1.17 mmol) as a yellow oil and tert-butyl N-methyl-N-(5,6,7,8-tetrahydro-4H-cyclohepta[c]thiophen-5-yl)carbamate enantiomer 2 (12b) (330 mg, 1.17 mmol) as a yellow oil. LCMS: m/z [M+H-Boc]⁺=226.0

### Procedure for preparation of N-methyl-5,6,7,8-tetrahydro-4H-cyclohepta[c]thiophen-5-amine hydrochloride enantiomer 1 (169)

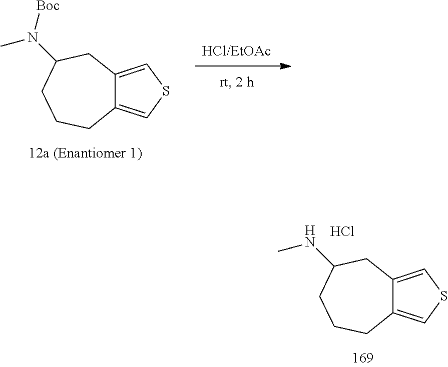 [SMILES: CN(C(=O)OC(C)(C)C)C1CCCc2cscc2C1 ; Cl ; CNC1CCCc2cscc2C1]

To a solution of tert-butyl N-methyl-N-(5, 6, 7, 8-tetrahydro-4H-cyclohepta[c]thiophen-5-yl) carbamate enantiomer 1 (12a) (330 mg, 1.17 mmol, 1 eq) in EtOAc (2 mL) was added HCl/EtOAc (4 M, 5 mL, 17.06 eq). The mixture was stirred at 25° C. for 2 hr. The reaction mixture was filtered and the cake was washed with EtOAc (10 mL) to yield N-methyl-5, 6, 7, 8-tetrahydro-4H-cyclohepta[c]thiophen-5-amine hydrochloride enantiomer 1 (169) (216.83 mg, 985.78 μmol, 84.06% yield) as an off-white solid. ¹H NMR (400 MHz, METHANOL-d4) δ 7.20 (d, J=3.2 Hz, 1H), 7.05 (d, J=3.1 Hz, 1H), 3.26-3.14 (m, 2H), 3.08-2.98 (m, 1H), 2.89-2.80 (m, 1H), 2.79-2.68 (m, 4H), 2.28-2.17 (m, 1H), 2.08-1.95 (m, 1H), 1.94-1.82 (m, 1H), 1.70-1.55 (m, 1H). LCMS: m/z [M+H]⁺=182.0

### Procedure for preparation of N-methyl-5,6,7,8-tetrahydro-4H-cyclohepta[c]thiophen-5-amine hydrochloride enantiomer 2 (170)

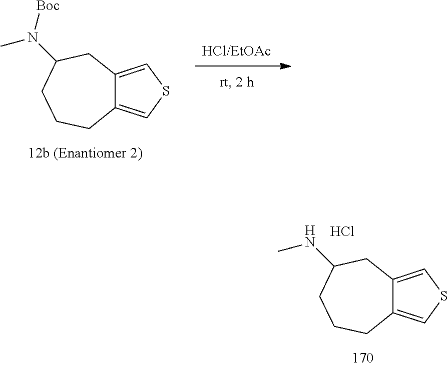 [SMILES: CN(C(=O)OC(C)(C)C)C1CCCc2cscc2C1 ; Cl ; CNC1CCCc2cscc2C1]

To a solution of tert-butyl N-methyl-N-(5, 6, 7, 8-tetrahydro-4H-cyclohepta[c]thiophen-5-yl) carbamate enantiomer 2 (12b) (330 mg, 1.17 mmol, 1 eq) in EtOAc (2 mL) was added HCl/EtOAc (4 M, 5 mL, 17.06 eq). The mixture was stirred at 25° C. for 2 hr. The reaction mixture was filtered and the cake was washed with EtOAc (10 mL) to yield N-methyl-5, 6, 7, 8-tetrahydro-4H-cyclohepta[c]thiophen-5-amine hydrochloride enantiomer 2 (170) (182.61 mg, 838.59 μmol, 71.51% yield) as a white solid. ¹H NMR (400 MHz, METHANOL-d4) δ 7.21 (d, J=3.2 Hz, 1H), 7.05 (d, J=3.2 Hz, 1H), 3.25-3.13 (m, 2H), 3.08-2.98 (m, 1H), 2.90-2.80 (m, 1H), 2.78-2.68 (m, 4H), 2.30-2.16 (m, 1H), 2.08-1.95 (m, 1H), 1.94-1.81 (m, 1H), 1.69-1.56 (m, 1H). LCMS: m/z [M+H]⁺=182.0 Synthetic scheme for synthesis of N-ethyl-4,5,6,7-tetrahydro-2-benzothiophen-5-amine hydrochloride enantiomer 1 (171) and enantiomer 2 (172)

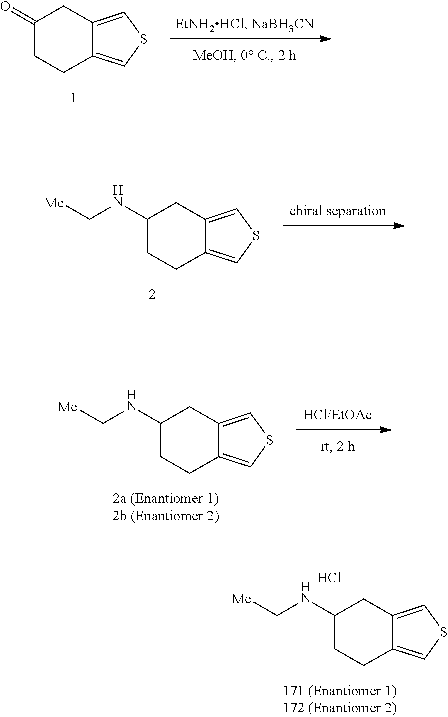 [SMILES: O=C1CCc2cscc2C1 ; CCNC1CCc2cscc2C1 ; CCNC1CCc2cscc2C1 ; Cl ; CCNC1CCc2cscc2C1]

### Procedure for preparation of N-ethyl-4,5,6,7-tetrahydro-2-benzothiophen-5-amine enantiomer 1 (2a) and enantiomer 2 (2b)

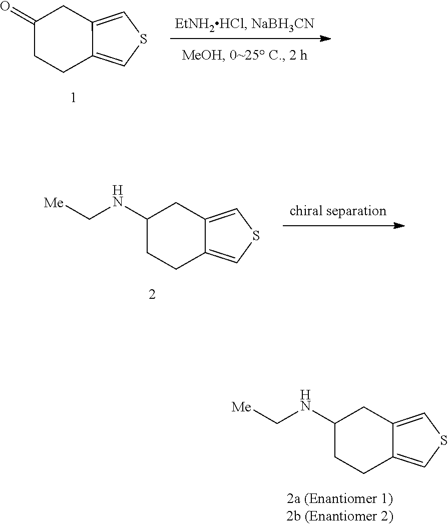 [SMILES: O=C1CCc2cscc2C1 ; CCNC1CCc2cscc2C1 ; CCNC1CCc2cscc2C1]

To a solution of 6,7-dihydro-4H-2-benzothiophen-5-one (1) (1 g, 6.57 mmol, 1 eq) and ethylamine hydrochloride (1.61 g, 19.7 mmol, 3 eq) in MeOH (20 mL) was added NaBH₃CN (2.06 g, 32.8 mmol, 5 eq) at 0° C. The mixture was stirred at 25° C. for 2 hr. The reaction mixture was poured into H₂O (20 mL) and was concentrated in vacuo to remove methanol. Extracted with EtOAc (50 mL*2). The combined organic phase was washed with brine (10 mL), dried over anhydrous sodium sulfate, filtered and concentrated under vacuum to give a residue. The residue was purified by flash silica gel chromatography (ISCO®; 40 g SepaFlash® Silica Flash Column, eluent 0~90% Petroleum ether:Ethyl acetate @40 mL/min) to afford racemic N-ethyl-4, 5, 6, 7-tetrahydro-2-benzothiophen-5-amine (2). LCMS: m/z [M+H]⁺=182.0. The racemic product was subjected to prep-Chiral-SFC (column: DAICEL CHIRALCEL OX (250 mm*30 mm, 10 μm); mobile phase: [0.1% NH₃H₂O EtOH]; B %: 35%-35%, 3.5 min) to afford the 1^{st} enantiomer 2a (RT=1.999 min, 220 mg, 1.21 mmol) and the 2^{nd} enantiomer 2b (RT=2.223 min, 200 mg, 1.10 mmol), both as yellow oils.

### Procedure for preparation of N-ethyl-4,5,6,7-tetrahydro-2-benzothiophen-5-amine hydrochloride enantiomer 1 (171)

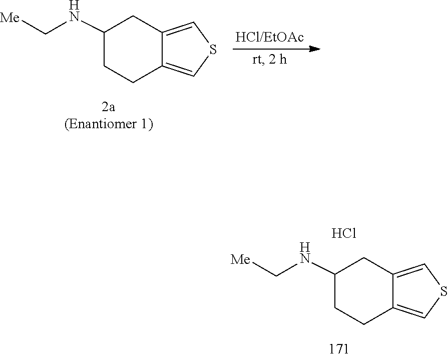 [SMILES: CCNC1CCc2cscc2C1 ; Cl ; CCNC1CCc2cscc2C1]

To a solution of N-ethyl-4,5,6,7-tetrahydro-2-benzothiophen-5-amine enantiomer 1 (2a) (220 mg, 1.21 mmol, 1 eq) in EtOAc (2 mL) was added HCl/EtOAc (4 M, 303 μL, 1.00 eq). The mixture was stirred at 25° C. for 2 hr. The mixture then filtered and the filter cake was dried under reduced pressure to afford N-ethyl-4,5,6,7-tetrahydro-2-benzothiophen-5-amine hydrochloride enantiomer 1 (171) (80.47 mg, 369.54 μmol, 30.45% yield as a white solid. LCMS: m/z [M+H]⁺=182.0. ¹H NMR (400 MHz, DEUTERIUM OXIDE) δ 7.12 (br s, 1H), 7.07 (br s, 1H), 3.59-3.48 (m, 1H), 3.28 (br dd, J=4.6, 16.3 Hz, 1H), 3.22-3.12 (m, 2H), 2.93 (td, J=5.1, 17.0 Hz, 1H), 2.82-2.69 (m, 2H), 2.27-2.15 (m, 1H), 1.90-1.77 (m, 1H), 1.28 (t, J=7.3 Hz, 3H)

### Procedure for preparation of N-ethyl-4,5,6,7-tetrahydro-2-benzothiophen-5-amine hydrochloride enantiomer 2 (172)

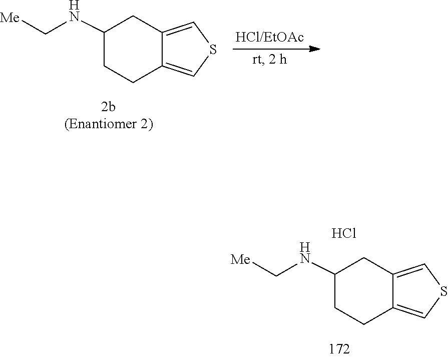 [SMILES: CCNC1CCc2cscc2C1 ; Cl ; CCNC1CCc2cscc2C1]

To a solution of N-ethyl-4,5,6,7-tetrahydro-2-benzothiophen-5-amine enantiomer 2 (2b) (200 mg, 1.10 mmol, 1 eq) in EtOAc (2 mL) was added HCl/EtOAc (4 M, 275.79 μL, 1.00 eq). The mixture was stirred at 25° C. for 2 hr and then filtered. The filter cake was dried under reduced pressure to afford N-ethyl-4, 5, 6, 7-tetrahydro-2-benzothiophen-5-amine hydrochloride enantiomer 2 (172) (171.76 mg, 776.93 μmol, 70.43% yield) as a white solid. LCMS: m/z [M+H]⁺=182.0. ¹H NMR (400 MHz, DEUTERIUM OXIDE) δ 7.10 (br s, 1H), 7.05 (s, 1H), 3.57-3.46 (m, 1H), 3.26 (dd, J=4.8, 15.5 Hz, 1H), 3.21-3.11 (m, 2H), 2.98-2.86 (m, 1H), 2.80-2.68 (m, 2H), 2.24-2.15 (m, 1H), 1.82 (dtd, J=5.6, 10.6, 13.0 Hz, 1H), 1.26 (t, J=7.3 Hz, 3H)

### Synthetic scheme for preparation of N-methyl-2-(trifluoromethyl)-4,5,6,7-tetrahydrobenzothiophen-5-amine hydrochloride (173)

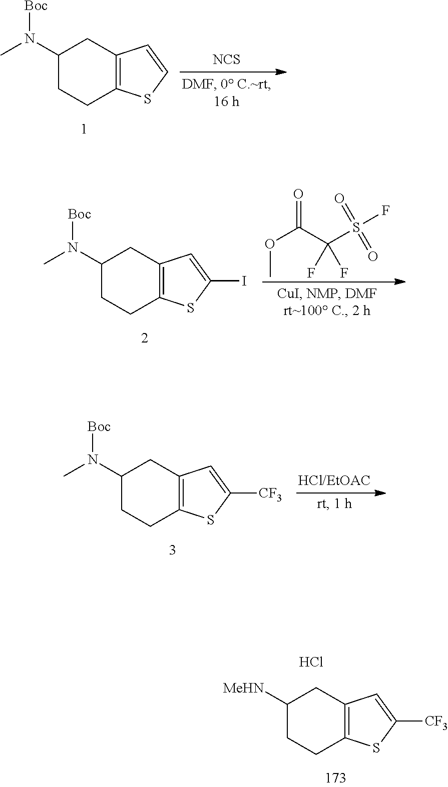

### Procedure for preparation of tert-butyl N-(2-iodo-4,5,6,7-tetrahydrobenzothiophen-5-yl)-N-methyl-carbamate (2)

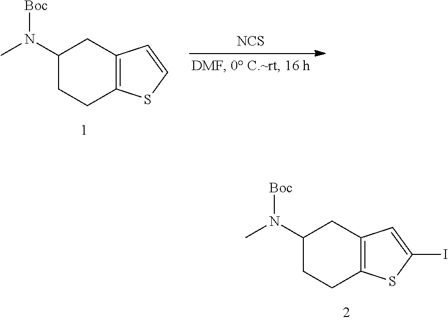 [SMILES: CN(C(=O)OC(C)(C)C)C1CCc2sccc2C1 ; "NCS" ; CN(C(=O)OC(C)(C)C)C1CCc2sc(I)cc2C1]

To a solution of tert-butyl N-methyl-N-(4,5,6,7-tetrahydrobenzothiophen-5-yl)carbamate (1) (0.75 g, 2.80 mmol, 1 eq) in DMF (20 mL) was added in portions NIS (1.89 g, 8.41 mmol, 3 eq) at 0° C. After addition, the mixture was stirred at 25° C. for 16 hr. The reaction mixture was poured into water (100 mL) and extracted with EtOAc (50 mL*3). The combined organic phases were washed with brine (50 mL), dried over anhydrous sodium sulfate, filtered and concentrated under vacuum. The residue was purified by flash silica gel chromatography (ISCO®; 20 g SepaFlash® Silica Flash Column, eluent 0-20% Ethyl acetate/Petroleum ether gradient @40 mL/min),to give tert-butyl N-(2-iodo-4,5,6,7-tetrahydrobenzothiophen-5-yl)-N-methyl-carbamate (2) (0.88 g, 2.24 mmol, 79.77% yield) as a brown oil. ¹H NMR (400 MHz, CHLOROFORM-d) δ 6.88 (s, 1H), 4.53-4.20 (m, 1H), 2.93-2.84 (m, 2H), 2.76-2.61 (m, 2H), 2.01-1.82 (m, 2H), 1.48 (s, 9H).

### Procedure for preparation of tert-butyl N-methyl-N-[2-(trifluoromethyl)-4,5,6,7-tetrahydrobenzothiophen-5-yl]carbamate (3)

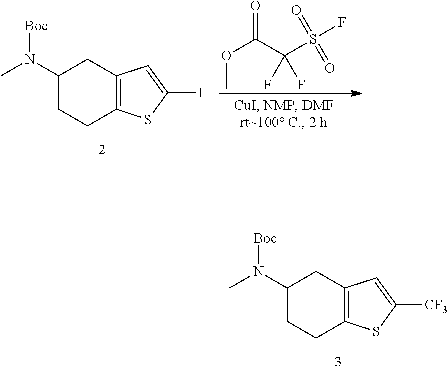

To a solution of tert-butyl N-(2-iodo-4,5,6,7-tetrahydrobenzothiophen-5-yl)-N-methyl-carbamate (2) (0.88 g, 2.24 mmol, 1 eq) in DMF (15 mL) was added methyl 2,2-difluoro-2-fluorosulfonyl-acetate (2.15 g, 11.19 mmol, 1.42 mL, 5 eq), NMP (1.11 g, 11.19 mmol, 1.09 mL, 5 eq) and CuI (639.22 mg, 3.36 mmol, 1.5 eq) at 25° C. The mixture was stirred at 100° C. for 2 hr. The reaction mixture was poured into H₂O (50 mL). The reaction was diluted with NH₃·H₂O (60 mL) and extracted with EtOAc mL (30 mL*3). The combined organic layers were washed with brine (20 mL*3), dried over anhydrous sodium sulfate, filtered and concentrated under reduced pressure. The residue was purified by flash silica gel chromatography (ISCO®; 12 g SepaFlash® Silica Flash Column, eluent 0~12% Ethyl acetate/Petroleum ether gradient @30 mL/min) to give tert-butyl N-methyl-N-[2-(trifluoromethyl)-4,5,6,7-tetrahydrobenzothiophen-5-yl]carbamate (3) (0.56 g, 1.67 mmol, 74.62% yield) as a light yellow oil. ¹H NMR (400 MHz, CHLOROFORM-d) δ 7.08 (s, 1H), 4.14 (s, 1H), 3.03-2.88 (m, 2H), 2.86-2.77 (m, 3H), 2.76-2.63 (m, 2H), 2.04-1.86 (m, 2H), 1.48 (s, 9H).

### Procedure for preparation of N-methyl-2-(trifluoromethyl)-4,5,6,7-tetrahydrobenzothiophen-5-amine hydrochloride (173)

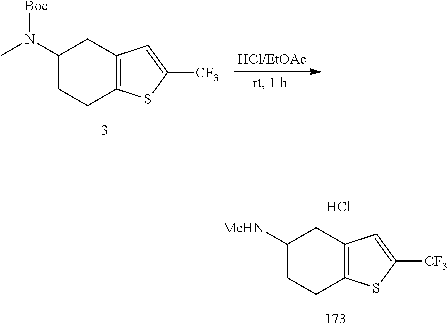 [SMILES: CN(C(=O)OC(C)(C)C)C1CCc2sc(C(F)(F)F)cc2C1 ; "HCl/EtOAc" ; Cl ; CNC1CCc2sc(C(F)(F)F)cc2C1]

To a solution of tert-butyl N-methyl-N-[2-(trifluoromethyl)-4,5,6,7-tetrahydrobenzothiophen-5-yl]carbamate (3) (0.1 g, 298.17 μmol, 1 eq) in EtOAc (2 mL) was added HCl/EtOAc (4 M, 2 mL, 26.83 eq). The mixture was stirred at 25° C. for 1 h. The reaction mixture was concentrated under vacuum to give N-methyl-2-(trifluoromethyl)-4,5,6,7-tetrahydrobenzothiophen-5-amine hydrochloride (173) (0.0645 g, 260.72 μmol, 87.44% yield) as a white solid. ¹H NMR (400 MHz, METHANOL-d₄) δ 7.29 (s, 1H), 3.69-3.43 (m, 1H), 3.27-3.15 (m, 1H), 3.10-2.93 (m, 2H), 2.86-2.72 (m, 4H), 2.45-2.28 (m, 1H), 2.03-1.89 (m, 1H). LCMS: m/z [M+H]⁺=236.1.

### Synthetic scheme for preparation of 5-(methylamino)-4,5,6,7-tetrahydrobenzothiophene-3-carbonitrile hydrochloride enantiomer 1 (174) and enantiomer 2 (175)

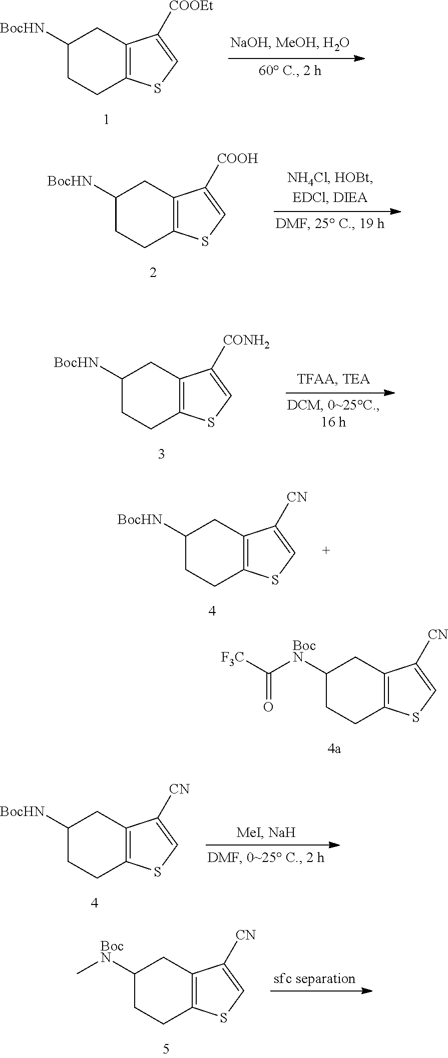

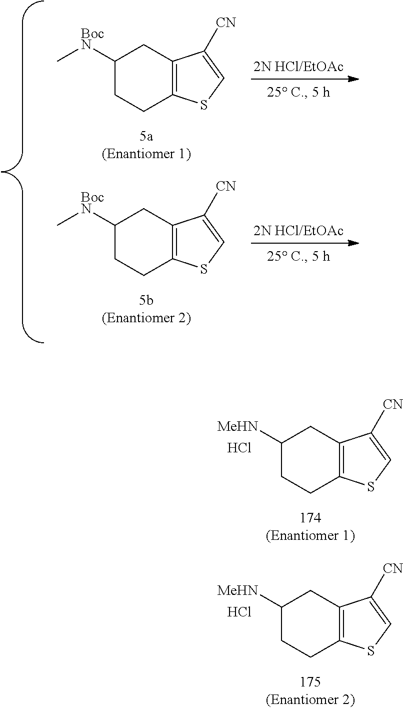 [SMILES: CN(C(=O)OC(C)(C)C)C1CCc2scc(C#N)c2C1 ; CN(C(=O)OC(C)(C)C)C1CCc2scc(C#N)c2C1 ; Cl ; CNC1CCc2scc(C#N)c2C1 ; Cl ; CNC1CCc2scc(C#N)c2C1]

### Procedure for preparation of 5-(tert-butoxycarbonylamino)-4,5,6,7-tetrahydrobenzothiophene-3-carboxylic acid (2)

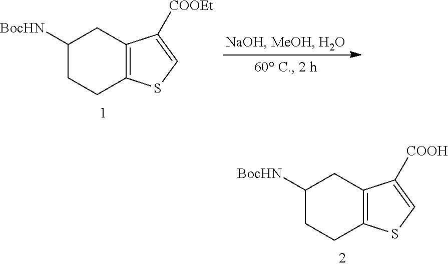 [SMILES: CCOC(=O)c1csc2c1CC(NC(=O)OC(C)(C)C)CC2 ; CC(C)(C)OC(=O)NC1CCc2scc(C(=O)O)c2C1]

To a solution of ethyl 5-(tert-butoxycarbonylamino)-4,5,6,7-tetrahydrobenzothiophene-3-carboxylate (1) (2.7 g, 8.30 mmol, 1 eq) in MeOH (20 mL) was added NaOH (995 mg, 24.89 mmol, 3 eq) and H₂O (10 mL). The mixture was stirred at 60° C. for 2 hr. The reaction mixture was concentrated under reduced pressure to remove MeOH. 1 M HCl aqueous solution was added into the mixture to adjust pH to 5, followed by filtration, the filter cake was dried to afford 5-(tert-butoxycarbonylamino)-4,5,6,7-tetrahydrobenzothiophene-3-carboxylic acid (2) (3.6 g, crude) as a yellow solid. ¹H NMR (400 MHz, METHANOL-d₄) δ 7.99 (s, 1H), 3.77 (dt, J=2.9, 11.0 Hz, 1H), 3.30-3.24 (m, 1H), 2.95-2.80 (m, 2H), 2.60 (br dd, J=9.3, 17.0 Hz, 1H), 2.11-2.02 (m, 1H), 1.81-1.68 (m, 1H), 1.46 (s, 9H)

### Procedure for preparation of tert-butyl N-(3-carbamoyl-4,5,6,7-tetrahydrobenzothiophen-5-yl)carbamate (3)

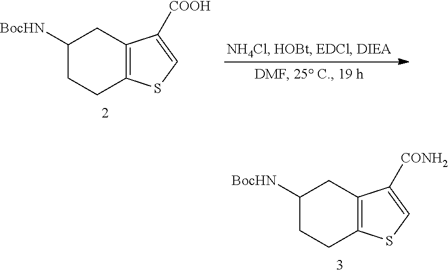 [SMILES: CC(C)(C)OC(=O)NC1CCc2scc(C(=O)O)c2C1 ; CC(C)(C)OC(=O)NC1CCc2scc(C(N)=O)c2C1]

To a solution of 5-(tert-butoxycarbonylamino)-4,5,6,7-tetrahydrobenzothiophene-3-carboxylic acid (2) (3.6 g, 12.11 mmol, 1 eq) in DMF (50 mL) was added EDCI (2.78 g, 14.53 mmol, 1.2 eq), HOBt (1.96 g, 14.53 mmol, 1.2 eq) and DIEA (4.69 g, 36.32 mmol, 6.33 mL, 3 eq) at 25° C. After addition, the mixture was stirred at this temperature for 10 min, and then NH₄Cl (1.94 g, 36.32 mmol, 3 eq) was added at 25° C. The resulting mixture was stirred at 25° C. for 19 hrs. The reaction mixture was poured into H₂O (300 mL) and extracted with EtOAc (100 mL*3). The combined organic phases were washed with brine (10 mL), dried over anhydrous sodium sulfate, filtered and concentrated under vacuum to give a residue. The residue was purified by flash silica gel chromatography (ISCO®; 25 g SepaFlash® Silica Flash Column, eluent 0~100% Ethyl acetate/Petroleum ether gradient @50 mL/min) to afford tert-butyl N-(3-carbamoyl-4,5,6,7-tetrahydrobenzothiophen-5-yl)carbamate (3) (2.6 g, 8.77 mmol, 72.46% yield) as yellow oil. ¹H NMR (400 MHz, CHLOROFORM-d) δ 7.55 (s, 1H), 5.78-5.40 (m, 1H), 4.81-4.47 (m, 1H), 4.06-3.92 (m, 1H), 3.23 (dd, J=5.0, 16.6 Hz, 1H), 2.89-2.84 (m, 2H), 2.71 (dd, J=7.9, 16.8 Hz, 1H), 2.18-2.06 (m, 1H), 1.92-1.81 (m, 1H), 1.46 (s, 9H)

### Procedure for preparation of tert-butyl N-(3-cyano-4,5,6,7-tetrahydrobenzothiophen-5-yl)carbamate (4) and tert-butyl N-(3-cyano-4,5,6,7-tetrahydrobenzothiophen-5-yl)-N-(2,2,2-trifluoroacetyl)carbamate (4a)

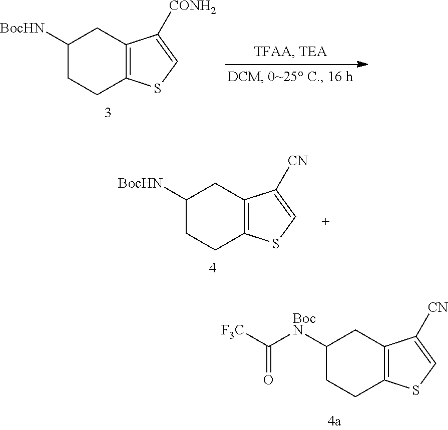 [SMILES: CC(C)(C)OC(=O)NC1CCc2scc(C(N)=O)c2C1 ; CC(C)(C)OC(=O)NC1CCc2scc(C#N)c2C1 ; CC(C)(C)OC(=O)N(C(=O)C(F)(F)F)C1CCc2scc(C#N)c2C1]

To a solution of tert-butyl N-(3-carbamoyl-4,5,6,7-tetrahydrobenzothiophen-5-yl)carbamate (3) (2.6 g, 8.77 mmol, 1 eq) in DCM (20 mL) was added dropwise TEA (2.66 g, 26.32 mmol, 3.66 mL, 3 eq) at 0° C. and then dropwise TFAA (3.68 g, 17.54 mmol, 2.44 mL, 2 eq) at 0° C. The resulting mixture was stirred at 25° C. for 16 hrs. The reaction mixture was poured into H₂O (50 mL) and extracted with EtOAc (20 mL*3). The combined organic phase was washed with brine (10 mL), dried over anhydrous sodium sulfate, filtered and concentrated under vacuum to give a residue. The residue was purified by flash silica gel chromatography (ISCO®; 25 g SepaFlash® Silica Flash Column, eluent 0~20% Ethyl acetate/Petroleum ether gradient @50 mL/min) to afford tert-butyl N-(3-cyano-4,5,6,7-tetrahydrobenzothiophen-5-yl)carbamate (4) (320 mg, 1.15 mmol, 13.10% yield) as a white solid and tert-butyl N-(3-cyano-4,5,6,7-tetrahydrobenzothiophen-5-yl)-N-(2,2,2-trifluoroacetyl)carbamate (4a) (1.2 g, 3.21 mmol, 36.54% yield) as a yellow oil. [4]¹H NMR (400 MHz, CHLOROFORM-d) δ 7.74 (s, 1H), 4.67-4.49 (m, 1H), 4.05 (br d, J=4.0 Hz, 1H), 3.11 (br dd, J=5.1, 16.3 Hz, 1H), 2.88 (br t, J=6.3 Hz, 2H), 2.56 (br dd, J=7.6, 16.4 Hz, 1H), 2.17-2.07 (m, 1H), 1.92-1.80 (m, 1H), 1.47 (s, 9H). [4a]¹H NMR (400 MHz, CHLOROFORM-d) δ 7.75 (s, 1H), 4.73-4.61 (m, 1H), 3.29-3.17 (m, 1H), 3.06-2.85 (m, 3H), 2.50 (dq, J=6.1, 12.2 Hz, 1H), 2.10-2.00 (m, 1H), 1.56 (s, 9H). ¹⁹F NMR (400 MHz, CHLOROFORM-d) δ-69.71

### Procedure for preparation of tert-butyl N-(3-cyano-4,5,6,7-tetrahydrobenzothiophen-5-yl)carbamate (4) from tert-butyl N-(3-cyano-4,5,6,7-tetrahydrobenzothiophen-5-yl)-N-(2,2,2-trifluoroacetyl)carbamate (4a)

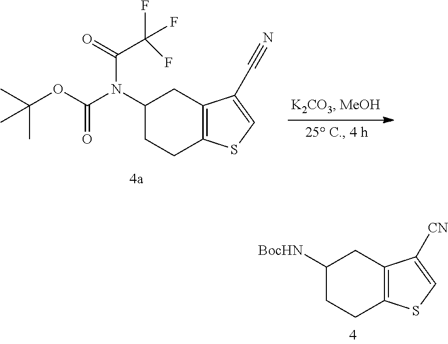 [SMILES: CC(C)(C)OC(=O)N(C(=O)C(F)(F)F)C1CCc2scc(C#N)c2C1 ; CC(C)(C)OC(=O)NC1CCc2scc(C#N)c2C1]

To a solution of tert-butyl N-(3-cyano-4,5,6,7-tetrahydrobenzothiophen-5-yl)-N-(2,2,2-trifluoroacetyl)carbamate (4a) (1.2 g, 3.21 mmol, 1 eq) in MeOH (10 mL) was added K₂CO₃ (886.02 mg, 6.41 mmol, 2 eq). The mixture was stirred at 25° C. for 4 hrs. The mixture was filtered and the filtrate was concentrated to give a residue. The residue was purified by flash silica gel chromatography (ISCO®; 12 g SepaFlash® Silica Flash Column, eluent 0~30% Ethyl acetate/Petroleum ether gradient @30 mL/min) to afford tert-butyl N-(3-cyano-4,5,6,7-tetrahydrobenzothiophen-5-yl)carbamate (4) (800 mg, 2.87 mmol, 89.66% yield).

### Procedure for preparation of tert-butyl N-(3-cyano-4,5,6,7-tetrahydrobenzothiophene-5-yl)-N-methyl-carbamate (5)

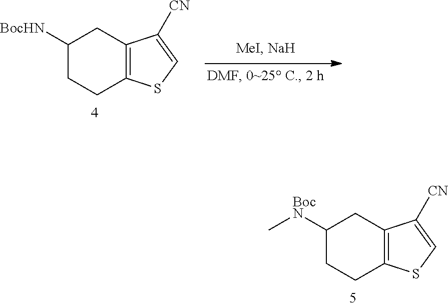 [SMILES: CC(C)(C)OC(=O)NC1CCc2scc(C#N)c2C1 ; CN(C(=O)OC(C)(C)C)C1CCc2scc(C#N)c2C1]

To a solution of tert-butyl N-(3-cyano-4,5,6,7-tetrahydrobenzothiophen-5-yl)carbamate (4) (320 mg, 1.15 mmol, 1 eq) in DMF (5 mL) was added NaH (91.96 mg, 2.30 mmol, 60% suspension in mineral oil, 2 eq) at 0° C. After addition, the mixture was stirred at this temperature for 0.5 hr, and then CH₃I (244.75 mg, 1.72 mmol, 107.35 μL, 1.5 eq) was added dropwise at 0° C. The resulting mixture was stirred at 25° C. for 1.5 hrs. The reaction mixture was poured into saturated solution NH₄Cl (20 mL) and extracted with EtOAc (20 mL*3). The combined organic phases were washed with brine (10 mL), dried over anhydrous sodium sulfate, filtered and concentrated under vacuum to give a residue. The residue was purified by prep-HPLC (column: Waters Xbridge 150*25 mm*5μm; mobile phase: [water(NH₄HCO₃)-ACN]; gradient:50%-80% B over 9 min) to afford tert-butyl N-(3-cyano-4,5,6,7-tetrahydrobenzothiophen-5-yl)-N-methyl-carbamate (5) (220 mg, 752.41 μmol, 65.45% yield) as a white solid. LCMS: m/z [M+H-tBu]⁺=237.2

### Procedure for preparation of tert-butyl N-(3-cyano-4,5,6,7-tetrahydrobenzothiophen-5-yl)-N-methyl-carbamate enantiomer 1 (5a) and enantiomer 2 (5b)

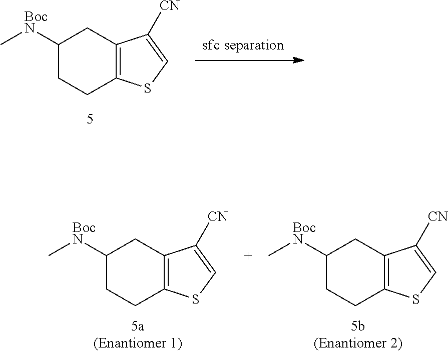 [SMILES: CN(C(=O)OC(C)(C)C)C1CCc2scc(C#N)c2C1 ; CN(C(=O)OC(C)(C)C)C1CCc2scc(C#N)c2C1 ; CN(C(=O)OC(C)(C)C)C1CCc2scc(C#N)c2C1]

Racemic tert-butyl N-(3-cyano-4,5,6,7-tetrahydrobenzothiophen-5-yl)-N-methyl-carbamate (5) (220 mg) was separated by SFC (column: DAICEL CHIRALPAK IG (250 mm*30 mm, 10 μm); mobile phase: [CO₂-EtOH (0.1% NH₃H₂O)]; B %:15%, isocratic elution mode) to afford tert-butyl N-(3-cyano-4,5,6,7-tetrahydrobenzothiophen-5-yl)-N-methyl-carbamate enantiomer 1 (5a) (100 mg, 342.00 μmol, 45.45% yield) as a white solid and tert-butyl N-(3-cyano-4,5,6,7-tetrahydrobenzothiophen-5-yl)-N-methyl-carbamate enantiomer 2 (5b) (90 mg, 307.80 μmol, 40.91% yield) also as a white solid.

### Procedure for preparation of 5-(methylamino)-4,5,6,7-tetrahydrobenzothiophene-3-carbonitrile hydrochloride enantiomer 1 (174)

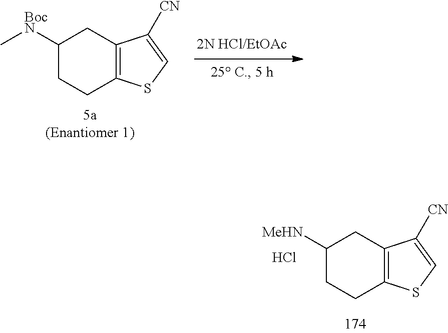 [SMILES: CN(C(=O)OC(C)(C)C)C1CCc2scc(C#N)c2C1 ; Cl ; CNC1CCc2scc(C#N)c2C1]

To a solution of tert-butyl N-(3-cyano-4,5,6,7-tetrahydrobenzothiophen-5-yl)-N-methyl-carbamate enantiomer 1 (5a) (100 mg, 342.00 μmol, 1 eq) in EtOAc (1 mL) was added HCl/EtOAc (4 M, 1 mL, 11.70 eq). The mixture was stirred at 25° C. for 5 hrs. The mixture was concentrated and residue was triturated with EtOAc (2 mL) at 25° C. for 10 min. The mixture was filtered and the filter cake was dried to afford 5-(methylamino)-4,5,6,7-tetrahydrobenzothiophene-3-carbonitrile hydrochloride enantiomer 1 (174) (63.75 mg, 275.91 μmol, 80.68% yield) as a white solid. ¹H NMR (400 MHz, METHANOL-d4) δ 8.14 (s, 1H), 3.63-3.54 (m, 1H), 3.25 (br d, J=5.4 Hz, 1H), 3.10-2.90 (m, 2H), 2.86-2.71 (m, 4H), 2.43-2.32 (m, 1H), 2.05-1.91 (m, 1H). LCMS: m/z [M+H]⁺=192.8.

### Procedure for preparation of 5-(methylamino)-4,5,6,7-tetrahydrobenzothiophene-3-carbonitrile hydrochloride enantiomer 2 (175)

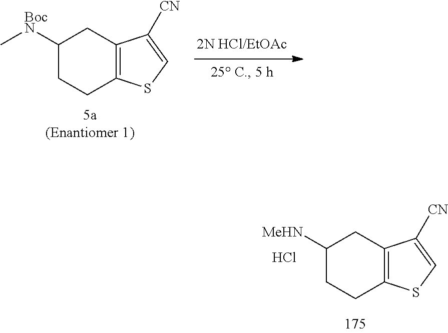 [SMILES: CN(C(=O)OC(C)(C)C)C1CCc2scc(C#N)c2C1 ; Cl ; CNC1CCc2scc(C#N)c2C1]

To a solution of tert-butyl N-(3-cyano-4,5,6,7-tetrahydrobenzothiophen-5-yl)-N-methyl-carbamate enantiomer 2 (5b) (90 mg, 307.80 μmol, 1 eq) in EtOAc (1 mL) was added HCl/EtOAc (4 M, 1 mL, 13.00 eq). The mixture was stirred at 25° C. for 5 hrs. The mixture was concentrated and the residue was triturated with EtOAc at 25° C. for 10 min. The mixture was filtered and the filter cake was dried to afford 5-(methylamino)-4,5,6,7-tetrahydrobenzothiophene-3-carbonitrile hydrochloride enantiomer 2 (175) (65.01 mg, 281.37 μmol, 91.41% yield) as a white solid. ¹H NMR (400 MHz, METHANOL-d4) δ 8.14 (s, 1H), 3.63-3.54 (m, 1H), 3.29-3.23 (m, 1H), 3.11-2.89 (m, 2H), 2.86-2.72 (m, 4H), 2.44-2.32 (m, 1H), 2.05-1.92 (m, 1H). LCMS: m/z [M+H]⁺=193.0

### Synthetic scheme for preparation of 5-amino-4,5,6,7-tetrahydrobenzo[b]thiophene-3-carbonitrile hydrochloride enantiomer 1 (176) and enantiomer 2 (177)

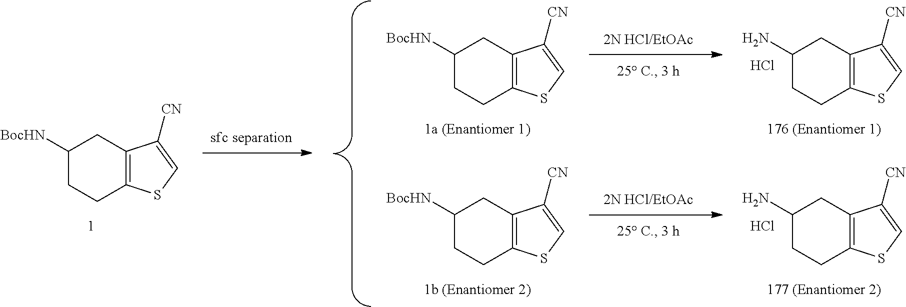

### Procedure for preparation of tert-butyl N-(3-cyano-4,5,6,7-tetrahydrobenzothiophen-5-yl)carbamate enantiomer 1 (1a) and enantiomer 2 (1b)

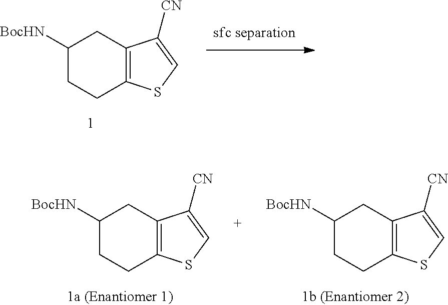 [SMILES: CC(C)(C)OC(=O)NC1CCc2scc(C#N)c2C1 ; CC(C)(C)OC(=O)NC1CCc2scc(C#N)c2C1 ; CC(C)(C)OC(=O)NC1CCc2scc(C#N)c2C1]

Racemic tert-butyl N-(3-cyano-4,5,6,7-tetrahydrobenzothiophen-5-yl)carbamate (1) (250 mg) was separated to its enantiomers 1a and 1b by SFC (column: DAICEL CHIRALPAK AD (250 mm*50 mm, 10 μm); mobile phase: [CO₂-EtOH (0.1% NH₃H₂O)]; B %:10%, isocratic elution mode). Enantiomer 1a was purified further by prep-HPLC (column: Waters Xbridge 150*25 mm*5 μm; mobile phase: [water(NH₄HCO₃)-ACN]; gradient:42%-72% B over 9 min). tert-butyl N-(3-cyano-4,5,6,7-tetrahydrobenzothiophen-5-yl)carbamate enantiomer 1 (1a) (90 mg, 320.08 μmol, 44.55% yield) was obtained as a white solid. Enantiomer 1b was also purified by prep-HPLC (column: Waters Xbridge 150*25 mm*5 μm; mobile phase: [water(NH₄HCO₃)-ACN]; gradient:42%-72% B over 9 min). tert-butyl N-(3-cyano-4,5,6,7-tetrahydrobenzothiophen-5-yl)carbamate enantiomer 2 (1b) (100 mg, 355.64 μmol, 49.50% yield) was obtained as a white solid.

### Procedure for preparation of 5-amino-4,5,6,7-tetrahydrobenzo[b]thiophene-3-carbonitrile hydrochloride enantiomer 1 (176)

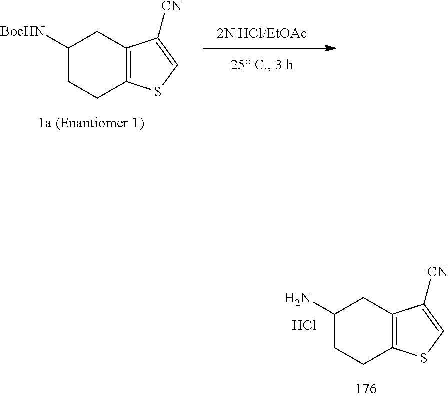 [SMILES: CC(C)(C)OC(=O)NC1CCc2scc(C#N)c2C1 ; Cl ; N#Cc1csc2c1CC(N)CC2]

To a solution of tert-butyl N-(3-cyano-4,5,6,7-tetrahydrobenzothiophen-5-yl)carbamate enantiomer 1 (1a) (90 mg, 323.31 μmol, 1 eq) in EtOAc (1 mL) was added HCl/EtOAc (4 M, 1 mL, 12.37 eq). The mixture was stirred at 25° C. for 3 hrs. The mixture was concentrated and the residue was triturated with EtOAc at 25° C. for 10 min. The mixture was filtered and the filter cake was dried to afford 5-amino-4,5,6,7-tetrahydrobenzo[b]thiophene-3-carbonitrile hydrochloride enantiomer 1 (176) (53.80 mg, 170.89 μmol, 52.85% yield) as a white solid. ¹H NMR (400 MHz, METHANOL-d4) δ=8.13 (s, 1H), 3.66 (dddd, J=3.1, 5.6, 8.8, 10.9 Hz, 1H), 3.20 (dd, J=5.5, 16.8 Hz, 1H), 3.08-2.88 (m, 2H), 2.79-2.67 (m, 1H), 2.35-2.23 (m, 1H), 2.05-1.89 (m, 1H). LCMS: m/z [M+H]⁺=179.0

### Procedure for preparation of 5-amino-4,5,6,7-tetrahydrobenzo[b]thiophene-3-carbonitrile hydrochloride enantiomer 2 (177)

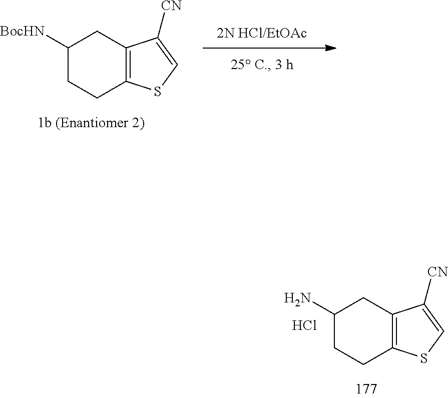 [SMILES: CC(C)(C)OC(=O)NC1CCc2scc(C#N)c2C1 ; Cl ; N#Cc1csc2c1CC(N)CC2]

To a solution of tert-butyl N-(3-cyano-4,5,6,7-tetrahydrobenzothiophen-5-yl)carbamate enantiomer 2 (1b) (100 mg, 359.24 μmol, 1 eq) in EtOAc (1 mL) was added HCl/EtOAc (4 M, 1.11 mL, 12.37 eq). The mixture was stirred at 25° C. for 3 hrs. The mixture was concentrated and the residue was triturated with EtOAc at 25° C. for 10 min. The mixture was filtered and the filter cake was dried to afford 5-amino-4,5,6,7-tetrahydrobenzo[b]thiophene-3-carbonitrile hydrochloride enantiomer 2 (177) (60.52 mg, 217.41 μmol, 60.52% yield) as a white solid. ¹H NMR (400 MHz, METHANOL-d4) δ=8.13 (s, 1H), 3.71-3.62 (m, 1H), 3.20 (dd, J=5.6, 16.2 Hz, 1H), 3.08-2.90 (m, 2H), 2.73 (tdd, J=1.8, 9.1, 16.0 Hz, 1H), 2.34-2.23 (m, 1H), 1.97 (dddd, J=6.2, 9.7, 10.7, 13.0 Hz, 1H). LCMS: m/z [M+H]⁺=179.1

### Synthetic scheme for preparation of N, 5-dimethyl-6,7-dihydro-4H-benzothiophen-5-amine hydrochloride enantiomer 1 (178) and enantiomer 2 (179)

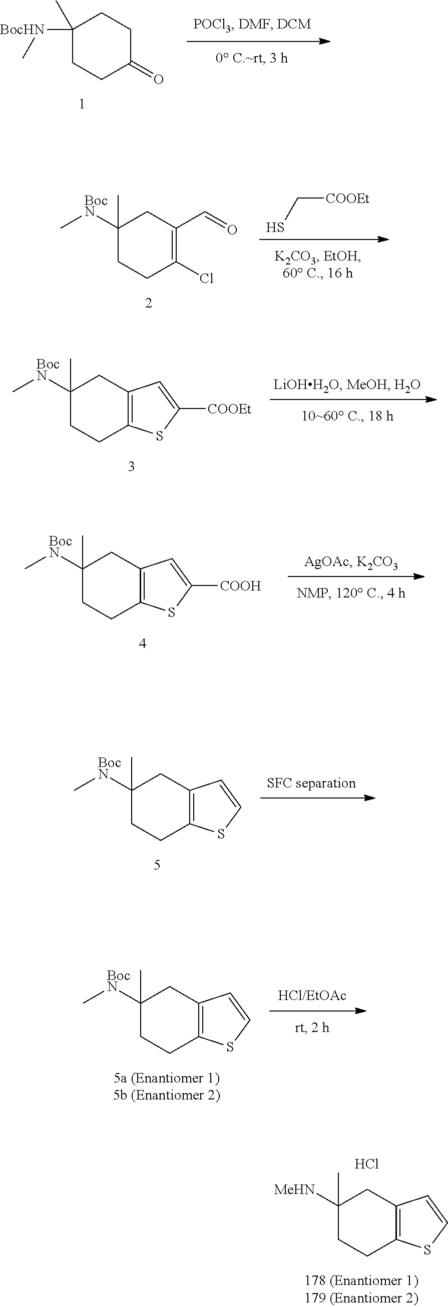

### Procedure for preparation of tert-butyl N-(4-chloro-3-formyl-1-methyl-cyclohex-3-en-1-yl)-N-methyl-carbamate (2)

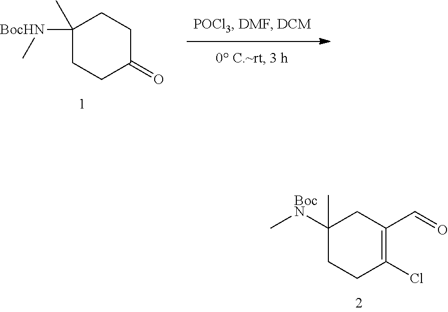

To a mixture of DMF (3.80 g, 52.0 mmol, 6.27 eq) in DCM (20 mL) was added POCl₃ (6.35 g, 41.4 mmol, 5.00 eq) at 0° C. The mixture was stirred at 10~15° C. for 1 h. Then a solution of tert-butyl N-methyl-N-(1-methyl-4-oxo-cyclohexyl)carbamate (1) (2.00 g, 8.29 mmol, 1.00 eq) in DCM (5 mL) was added at 0° C. and the mixture was stirred at 0° C. for 1 h. Then the reaction mixture was warmed to room temperature and stirred for 1 h and then poured into sat. NaHCO₃ (20 mL) and separated. The aqueous phase was extracted with EtOAc (10 mL*3). The combined organic layers were dried over anhydrous sodium sulfate, filtered and concentrated under vacuum to give a residue. The residue was purified by flash silica gel chromatography (ISCO®; 20 g SepaFlash® Silica Flash Column, eluent 0-13% Ethyl acetate/Petroleum ether gradient @40 mL/min) to give tert-butyl N-(4-chloro-3-formyl-1-methyl-cyclohex-3-en-1-yl)-N-methyl-carbamate (2) (1.60 g, 5.56 mmol, 67.1% yield) as a colorless oil. ¹H NMR (400 MHz, CDCl₃-d) δ 10.18 (s, 1H), 3.04-2.91 (m, 2H), 2.71 (s, 3H), 2.68-2.50 (m, 2H), 2.18-2.08 (m, 1H), 1.79-1.67 (m, 1H), 1.47 (s, 9H), 1.36 (s, 3H).

### Procedure for preparation of ethyl 5-[tert-butoxycarbonyl(methyl)amino]-5-methyl-6,7-dihydro-4H-benzothiophene-2-carboxylate (3)

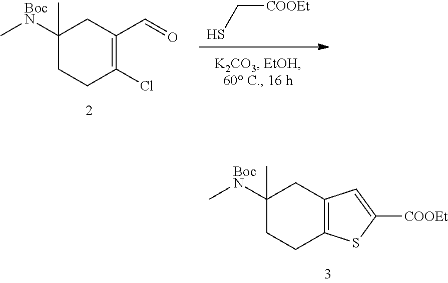 [SMILES: CN(C(=O)OC(C)(C)C)C1(C)CCC(Cl)=C(C=O)C1 ; CCOC(=O)CS ; CCOC(=O)c1cc2c(s1)CCC(C)(N(C)C(=O)OC(C)(C)C)C2]

To a mixture of tert-butyl N-(4-chloro-3-formyl-1-methyl-cyclohex-3-en-1-yl)-N-methyl-carbamate (2) (1.60 g, 5.56 mmol, 1.00 eq) and K₂CO₃ (1.15 g, 8.34 mmol, 1.50 eq) in EtOH (10 mL) was added ethyl 2-sulfanylacetate (0.8 g, 6.66 mmol, 1.20 eq). The resulting mixture was stirred at 60° C. for 16 h. The reaction mixture was filtered and the cake was washed with EtOAc (20 mL). The combined organic phases were concentrated under vacuum to give a residue. The residue was purified by flash silica gel chromatography (ISCO®; 12 g SepaFlash® Silica Flash Column, eluent 0~9% Ethyl acetate/Petroleum ether gradient @30 mL/min) to give ethyl 5-[tert-butoxycarbonyl(methyl)amino]-5-methyl-6,7-dihydro-4H-benzothiophene-2-carboxylate (3) (1.75 g, 4.95 mmol, 89.1% yield) as a colorless oil. ¹H NMR (400 MHz, CDCl₃-d) δ 7.44 (s, 1H), 4.32 (q, J=7.2 Hz, 2H), 3.12 (d, J=16.8 Hz, 1H), 3.03-2.92 (m, 1H), 2.79 (t, J=6.0 Hz, 2H), 2.73 (s, 3H), 2.65 (d, J=16.4 Hz, 1H), 1.80 (td, J=7.6, 13.2 Hz, 1H), 1.46 (s, 9H), 1.42 (s, 3H), 1.36 (t, J=7.2 Hz, 3H).

### Procedure for preparation of 5-[tert-butoxycarbonyl(methyl)amino]-5-methyl-6,7-dihydro-4H-benzothiophene-2-carboxylic acid (4)

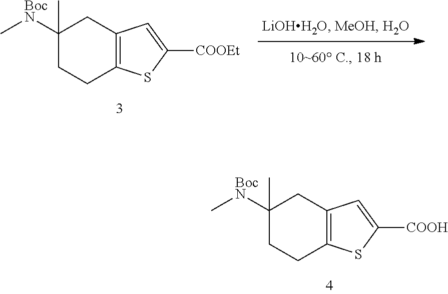 [SMILES: CCOC(=O)c1cc2c(s1)CCC(C)(N(C)C(=O)OC(C)(C)C)C2 ; CN(C(=O)OC(C)(C)C)C1(C)CCc2sc(C(=O)O)cc2C1]

To a solution of ethyl 5-[tert-butoxycarbonyl(methyl)amino]-5-methyl-6,7-dihydro-4H-benzothiophene-2-carboxylate (3) (1.75 g, 4.95 mmol, 1.00 eq) in MeOH (10 ml) and H₂O (10 ml) was added LiOH·H₂O (623 mg, 14.9 mmol, 3.00 eq). The resulting mixture was stirred at 10-15° C. for 16 h. The reaction mixture was heated to 60° C. and stirred for 2 h. The reaction mixture was concentrated under vacuum to remove MeOH, and extracted with EtOAc (15 mL). The aqueous phase was adjusted pH to ~7 with HCl (1 mol/L in water), then adjusted pH to 3-4 with aq. citric acid and filtered. The cake was washed with water (20 mL) and collected under vacuum to give 5-[tert-butoxycarbonyl(methyl)amino]-5-methyl-6,7-dihydro-4H-benzothiophene-2-carboxylic acid (4) (1.5 g, 4.61 mmol, 93.1% yield) as a white solid which was used into next step without further purification. ¹H NMR (400 MHz, CDCl₃-d) δ 7.53 (s, 1H), 3.16 (d, J=16.4 Hz, 1H), 3.04-2.94 (m, 1H), 2.82 (t, J=6.4 Hz, 2H), 2.74 (s, 3H), 2.67 (d, J=16.8 Hz, 1H), 1.81 (td, J=8.0, 13.2 Hz, 1H), 1.47 (s, 9H), 1.43 (s, 3H).

### Procedure for preparation of tert-butyl N-methyl-N-(5-methyl-6,7-dihydro-4H-benzothiophen-5-yl)carbamate (5)

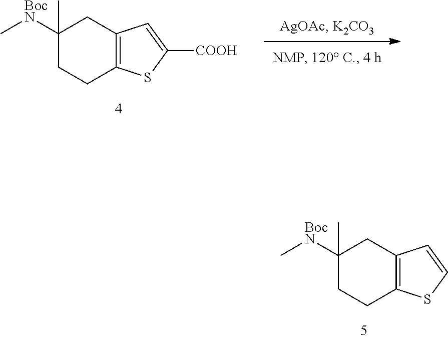 [SMILES: CN(C(=O)OC(C)(C)C)C1(C)CCc2sc(C(=O)O)cc2C1 ; CN(C(=O)OC(C)(C)C)C1(C)CCc2sccc2C1]

To a mixture of 5-[tert-butoxycarbonyl(methyl)amino]-5-methyl-6,7-dihydro-4H-benzothiophene-2-carboxylic acid (4) (700 mg, 2.15 mmol, 1.00 eq) and K₂CO₃ (892 mg, 6.45 mmol, 3.00 eq) in NMP (10 mL) was added AgOAc (54 mg, 323 μmol, 0.15 eq). The resulting mixture was stirred at 120° C. for 2 hr. Additional AgOAc (71.8 mg, 430.21 μmol, 0.2 eq) was added to the reaction mixture and the reaction mixture was stirred at 120° C. for 2 h. The reaction mixture was diluted with EtOAc (20 mL) and filtered. The filtrate was diluted with water (50 mL) and separated. The aqueous phase was extracted with EtOAc (15 mL*2). The combined organic phases were washed with brine (20 mL), dried over anhydrous sodium sulfate, filtered and concentrated under vacuum to give a residue. The residue was purified by flash silica gel chromatography (ISCO®; 12 g SepaFlash® Silica Flash Column, eluent 0~7% Ethyl acetate/Petroleum ether gradient @30 mL/min) to give tert-butyl N-methyl-N-(5-methyl-6,7-dihydro-4H-benzothiophen-5-yl)carbamate (5) (500 mg, 1.78 mmol, 82.6% yield) as a white solid. ¹H NMR (400 MHz, CDCl₃-d) δ 7.07 (d, J=5.2 Hz, 1H), 6.71 (d, J=5.2 Hz, 1H), 3.10 (d, J=16.4 Hz, 1H), 2.96-2.85 (m, 1H), 2.82-2.74 (m, 5H), 2.71 (d, J=16.4 Hz, 1H), 1.82 (td, J=7.2, 13.2 Hz, 1H), 1.47 (s, 9H), 1.43 (s, 3H). LCMS: m/z [M-t-Bu+H]⁺=226.0.

### Procedure for preparation of tert-butyl N-methyl-N-(5-methyl-6,7-dihydro-4H-benzothiophen-5-yl)carbamate enantiomer 1 (5a) and enantiomer 2 (5b)

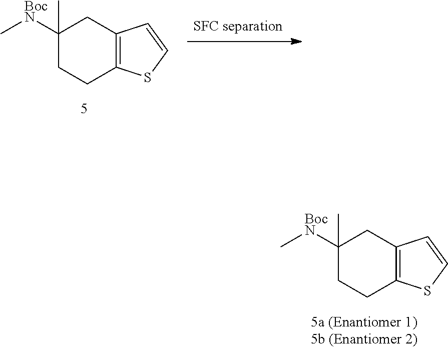 [SMILES: CN(C(=O)OC(C)(C)C)C1(C)CCc2sccc2C1 ; CN(C(=O)OC(C)(C)C)C1(C)CCc2sccc2C1]

Racemic tert-butyl N-methyl-N-(5-methyl-6,7-dihydro-4H-benzothiophen-5-yl)carbamate (5) (250 mg, 711 μmol, 1.00 eq) was separated by SFC (column: DAICEL CHIRALCEL OD (250 mm*50 mm, 1 0 m); mobile phase: [Hexane-EtOH (0.1% NH₃·H₂O)]; B %: 10%, isocratic elution mode) to give tert-butyl N-methyl-N-(5-methyl-6,7-dihydro-4H-benzothiophen-5-yl)carbamate enantiomer 1 (5a) (110 mg, 390.88 μmol, 55.0% yield) as a white solid and tert-butyl N-methyl-N-(5-methyl-6,7-dihydro-4H-benzothiophen-5-yl)carbamateenantiomer 2 (5b) (90 mg, 319.81 μmol, 45.0% yield) as a white solid.

### Procedure for preparation of N, 5-dimethyl-6,7-dihydro-4H-benzothiophen-5-amine hydrochloride enantiomer 1 (178)

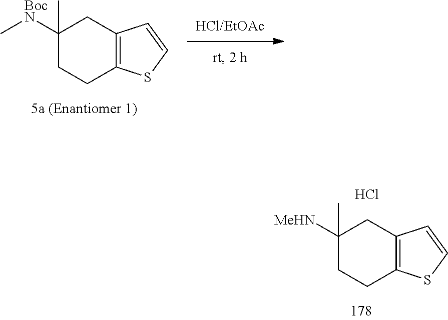 [SMILES: CN(C(=O)OC(C)(C)C)C1(C)CCc2sccc2C1 ; Cl ; CNC1(C)CCc2sccc2C1]

To a solution of tert-butyl N-methyl-N-(5-methyl-6,7-dihydro-4H-benzothiophen-5-yl)carbamate enantiomer 1 (5a) (110 mg, 390 μmol, 1.00 eq) in EtOAc (5 mL) was added HCl/EtOAc (4 M, 5 mL). The reaction mixture was stirred at rt for 2 h. The reaction mixture was filtered. The cake was washed with EtOAc (10 mL) and dried over vacuum to give N, 5-dimethyl-6,7-dihydro-4H-benzothiophen-5-amine hydrochloride enantiomer 1 (178) (45.47 mg, 209 μmol, 53.37% yield) as a white solid. ¹H NMR (400 MHz, MeOD-d₄) δ 7.28 (d, J=5.2 Hz, 1H), 6.82 (d, J=5.2 Hz, 1H), 3.02-2.94 (m, 2H), 2.92 (s, 2H), 2.73 (s, 3H), 2.11 (t, J=6.8 Hz, 2H), 1.42 (s, 3H). LCMS: m/z [M+H]⁺=182.0.

### Procedure for preparation of N, 5-dimethyl-6,7-dihydro-4H-benzothiophen-5-amine hydrochloride enantiomer 2 (179)

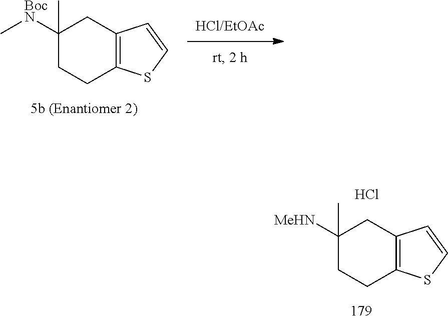 [SMILES: CN(C(=O)OC(C)(C)C)C1(C)CCc2sccc2C1 ; Cl ; CNC1(C)CCc2sccc2C1]

To a solution of tert-butyl N-methyl-N-(5-methyl-6,7-dihydro-4H-benzothiophen-5-yl)carbamate enantiomer 2 (5b) (90 mg, 320 μmol, 1.00 eq) in EtOAc (5 mL) was added HCl/EtOAc (4 M, 5 mL). The resulting mixture was stirred at rt for 2 h. The reaction mixture was filtered. The cake was washed with EtOAc (10 mL) and dried over vacuum to give N, 5-dimethyl-6,7-dihydro-4H-benzothiophen-5-amine hydrochloride enantiomer 2 (179) (51.12 mg, 234.00 μmol, 73.17% yield) as a white solid. ¹H NMR (400 MHz, MeOD-d₄) δ 7.28 (d, J=5.2 Hz, 1H), 6.82 (d, J=4.8 Hz, 1H), 3.02-2.94 (m, 2H), 2.92 (s, 2H), 2.73 (s, 3H), 2.12 (t, J=6.8 Hz, 2H), 1.42 (s, 3H). LCMS: m/z [M+H]⁺=182.0.

### Synthetic scheme for preparation of N-methyl-3-(trifluoromethyl)-4,5,6,7-tetrahydrobenzothiophen-6-amine hydrochloride (180)

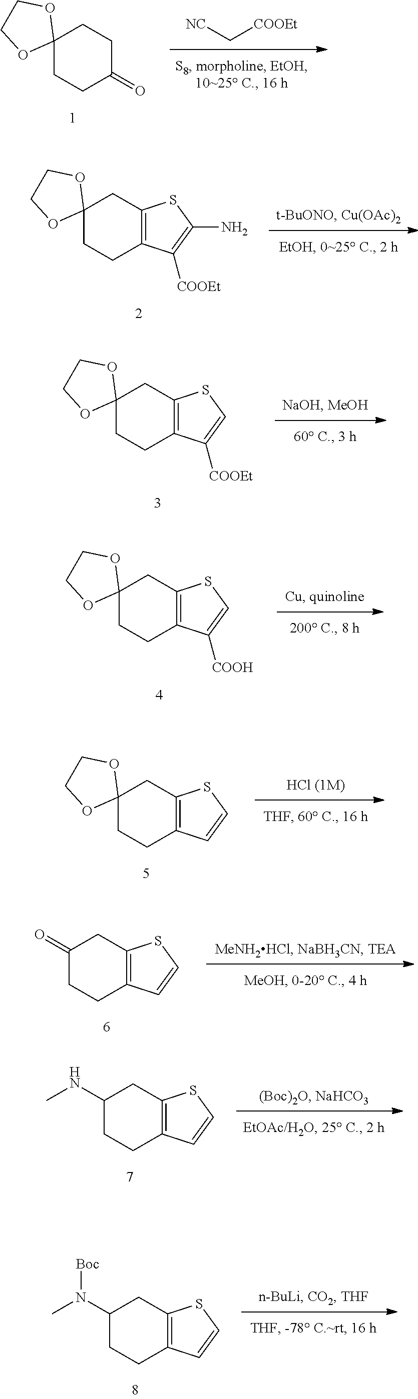

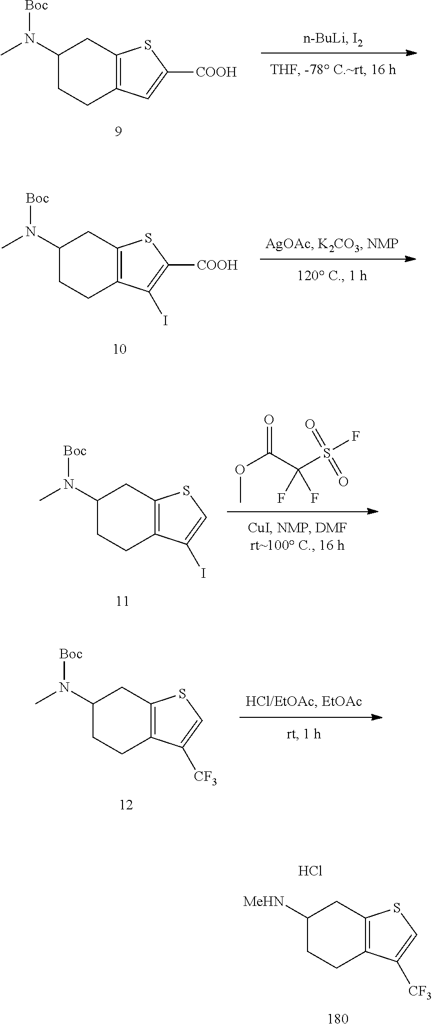

### Procedure for preparation of ethyl 2′-aminospiro[1,3-dioxolane-2,6′-5,7-dihydro-4H-benzothiophene]-3′-carboxylate (2)

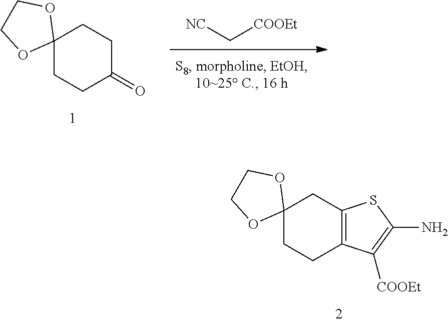 [SMILES: O=C1CCC2(CC1)OCCO2 ; CCOC(=O)CC#N ; CCOC(=O)c1c(N)sc2c1CCC1(C2)OCCO1]

To a mixture of 1,4-dioxaspiro[4.5]decan-8-one (1) (100 g, 640.29 mmol, 1 eq), S8 (22.59 g, 704.32 mmol, 1.1 eq) and ethyl cyanoformate (69.79 g, 704.32 mmol, 69.03 mL, 1.1 eq) in EtOH (1000 mL) was added morpholine (61.36 g, 704.32 mmol, 61.98 mL, 1.1 eq) in one portion at 25° C. under N₂. The mixture was stirred at 25° C. for 16 h. The mixture was quenched with H₂O (50 mL) and filtered and concentrated at reduced pressure to give a residue. The mixture was diluted with water 500 mL and then extracted with EtOAc (300 mL*3). The combined organic phases were washed with brine (200 mL*3), dried over Na₂SO₄, filtered and concentrated at reduced pressure to give a residue. The residue was purified by silica gel chromatography (100-200 mesh silica gel, Petroleum ether/Ethyl acetate=1/0~1/1, Rf=0.26). Compound ethyl 2′-aminospiro[1,3-dioxolane-2,6′-5,7-dihydro-4H-benzothiophene]-3′-carboxylate (2) (170.8 g, 602.80 mmol, 94.15% yield) was obtained as a yellow oil. ¹H NMR (400 MHz, CHLOROFORM-d) δ 4.26 (q, J=7.1 Hz, 2H), 4.02 (s, 4H), 2.93 (tt, J=1.6, 6.5 Hz, 2H), 2.74 (s, 2H), 1.90 (t, J=6.5 Hz, 2H), 1.33 (t, J=7.1 Hz, 3H).

### Procedure for preparation of ethyl spiro[1,3-dioxolane-2,6′-5,7-dihydro-4H-benzothiophene]-3′-carboxylate (3)

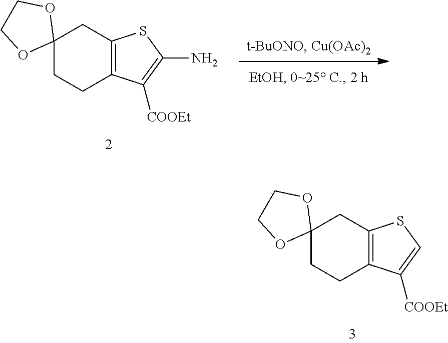 [SMILES: CCOC(=O)c1c(N)sc2c1CCC1(C2)OCCO1 ; CCOC(=O)c1csc2c1CCC1(C2)OCCO1]

To a mixture of ethyl 2′-aminospiro[1,3-dioxolane-2,6′-5,7-dihydro-4H-benzothiophene]-3′-carboxylate (2) (56.5*3 g, 199.41 mmol, 1 eq) and Cu(OAc)₂ (39.84 g, 219.35 mmol, 1.1 eq) in EtOH (1500 mL) was added t-BuONO (20.56 g, 199.41 mmol, 23.72 mL, 1 eq) at 0° C. under N₂. The mixture was stirred at 25° C. for 2 hours. The reaction mixture was concentrated under vacuum to give a residue. The residue was purified by flash silica gel chromatography (ISCO®; 3000 g SepaFlash® Silica Flash Column, eluent 0~33% Ethyl acetate/Petroleum ether gradient @500 mL/min). Compound ethyl spiro [1, 3-dioxolane-2,6′-5,7-dihydro-4H-benzothiophene]-3′-carboxylate (3) (112.53 g, 419.37 mmol, 70.10% yield) was obtained as a yellow solid. ¹H NMR (400 MHz, CHLOROFORM-d) δ 7.95 (s, 1H), 4.29 (q, J=7.1 Hz, 2H), 4.05 (s, 4H), 3.16-3.05 (m, 2H), 2.99 (s, 2H), 1.96 (t, J=6.6 Hz, 2H), 1.35 (t, J=7.1 Hz, 3H).

### Procedure for preparation of spiro [1,3-dioxolane-2,6′-5,7-dihydro-4H-benzothiophene]-3′-carboxylic acid (4)

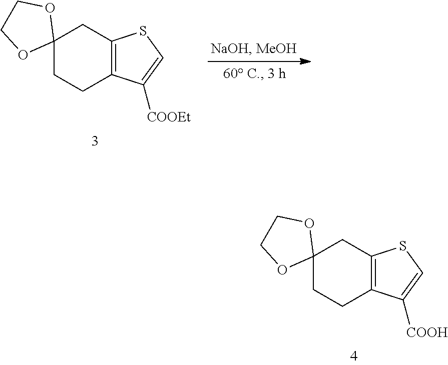 [SMILES: CCOC(=O)c1csc2c1CCC1(C2)OCCO1 ; O=C(O)c1csc2c1CCC1(C2)OCCO1]

To a solution of ethyl spiro[1,3-dioxolane-2,6′-5,7-dihydro-4H-benzothiophene]-3′-carboxylate (3) (56*2 g, 208.70 mmol, 1 eq) in MeOH (600 mL) was added aq. NaOH (3 M, 208.70 mL, 3 eq). The mixture was stirred at 60° C. for 3 hr. Most of the solvent was removed under reduced pressure. The pH value of the solution was adjusted to 5-6 by aq. 1 M HCl. The mixture was then diluted with H₂O mL 1000 mL and extracted with EtOAc (500 mL*3). The combined organic layers were washed with brine 500 mL, dried over anhydrous sodium sulfate, filtered and concentrated under reduced pressure to give compound spiro[1,3-dioxolane-2,6′-5,7-dihydro-4H-benzothiophene]-3′-carboxylic acid (4) (95 g, crude) as a white solid. ¹H NMR (400 MHz, CHLOROFORM-d) δ 8.08 (s, 1H), 4.05 (s, 4H), 3.19-2.91 (m, 4H), 1.97 (br t, J=6.5 Hz, 2H).

### Procedure for preparation of spiro[1,3-dioxolane-2,6′-5,7-dihydro-4H-benzothiophene](5)

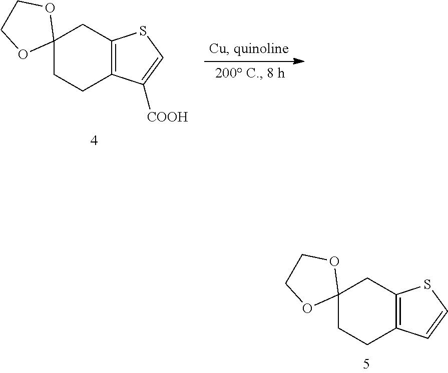 [SMILES: O=C(O)c1csc2c1CCC1(C2)OCCO1 ; c1cc2c(s1)CC1(CC2)OCCO1]

To a solution of spiro[1,3-dioxolane-2,6′-5,7-dihydro-4H-benzothiophene]-3′-carboxylic acid (4) (40 g, 166.48 mmol, 1 eq) in quinoline (300 mL) was added Cu (26.45 g, 416.19 mmol, 2.95 mL, 2.5 eq) at 25° C. The mixture was stirred at 200° C. for 8 hr under N₂. The mixture was filtered, the filtrate pH was adjusted to 6 with aq. 1M HCl, and extracted with EtOAc (300 mL*3). The combined organic phases were dried over anhydrous sodium sulfate, filtered and concentrated under vacuum to give a residue. The residue was purified by flash silica gel chromatography (ISCO®; 330 g SepaFlash® Silica Flash Column, eluent 0-10% Ethyl acetate/Petroleum ether gradient @120 mL/min). Compound spiro[1,3-dioxolane-2,6′-5,7-dihydro-4H-benzothiophene](5) (27.4 g, 139.61 mmol, 83.86% yield) was obtained as yellow oil. ¹H NMR (400 MHz, CHLOROFORM-d) δ 7.09 (d, J=5.0 Hz, 1H), 6.78 (d, J=5.0 Hz, 1H), 4.05 (s, 4H), 3.00 (s, 2H), 2.84 (br t, J=6.3 Hz, 2H), 1.97 (t, J=6.4 Hz, 2H).

### Procedure for preparation of 5, 7-dihydro-4H-benzothiophen-6-one (6)

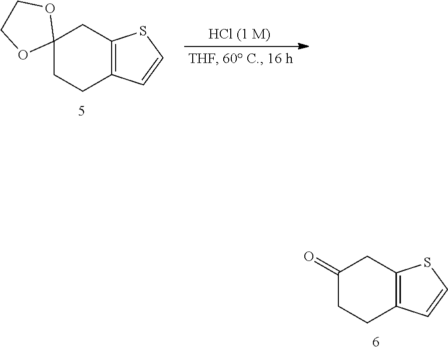 [SMILES: c1cc2c(s1)CC1(CC2)OCCO1 ; O=C1CCc2ccsc2C1]

To a solution of spiro[1,3-dioxolane-2,6′-5,7-dihydro-4H-benzothiophene](5) (25 g, 127.38 mmol, 1 eq) in THF (250 mL) was added aq. HCl (1 M, 242.02 mL, 1.9 eq). The mixture was stirred at 60° C. for 16 hr. The reaction mixture was concentrated under reduced pressure to remove most of the solvent. Then followed dilution with H₂O (150 mL) and the mixture was washed with EtOAc (50 mL*3). The combined organic layers were washed with brine (100 mL), dried over anhydrous sodium sulfate, filtered and concentrated under reduced pressure to give a residue. The residue was purified by flash silica gel chromatography (ISCO®; 80 g SepaFlash® Silica Flash Column, eluent 0~15% Ethyl acetate/Petroleum ether gradient @60 mL/min). Compound 5,7-dihydro-4H-benzothiophen-6-one (6) (20 g) was obtained as a yellow oil. ¹H NMR (400 MHz, CHLOROFORM-d) δ 7.17 (d, J=5.1 Hz, 1H), 6.84 (d, J=5.1 Hz, 1H), 3.63 (s, 2H), 3.02 (t, J=6.8 Hz, 2H), 2.68 (t, J=6.9 Hz, 2H).

### Procedure for preparation of N-methyl-4,5,6,7-tetrahydrobenzothiophen-6-amine (7)

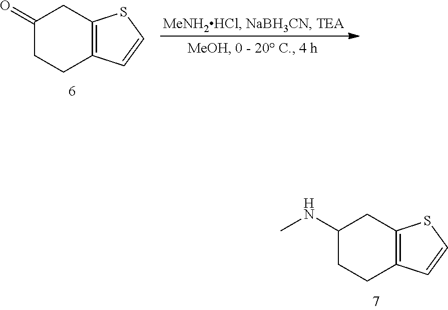

A mixture of 5,7-dihydro-4H-benzothiophen-6-one (6) (18 g, 118.26 mmol, 1 eq), methylamine hydrochloride (11.02 g, 354.77 mmol, 3 eq), TEA (23.93 g, 236.51 mmol, 32.92 mL, 2 eq) in MeOH (200 mL) stirred at 0° C. for 0.5 h. Followed addition of NaBH₃CN (37.16 g, 591.28 mmol, 5 eq) in portions slowly under nitrogen. The mixture was warmed up to 20° C. and stirred for 3.5 hr under N₂ atmosphere. The reaction mixture was quenched with water 10 mL dropwise at 0° C. Then the mixture was concentrated under reduced pressure to remove solvent. The residue was diluted with H₂O (200 mL) and extracted with EtOAc (100 mL*4). The combined organic layers were washed with brine (200 mL), dried over anhydrous sodium sulfate, filtered and concentrated under reduced pressure to give compound N-methyl-4,5,6,7-tetrahydrobenzothiophen-6-amine (7) (17.5 g, 82.13 mmol, 69.45% yield as a black brown oil. ¹H NMR (400 MHz, CHLOROFORM-d) δ 7.12 (d, J=5.1 Hz, 1H), 6.82-6.70 (m, 1H), 5.20 (br d, J=1.4 Hz, 2H), 3.34-3.20 (m, 2H), 2.95-2.79 (m, 2H), 2.78-2.60 (m, 4H), 2.29-2.17 (m, 1H), 1.98-1.85 (m, 1H). LCMS: m/z [M+H]⁺=168.2.

### Procedure for preparation of tert-butyl methyl(4,5,6,7-tetrahydrobenzo[b]thiophen-6-yl)carbamate (8)

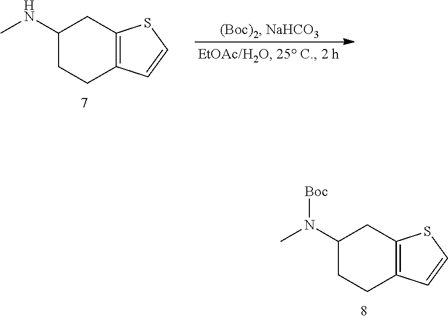

To a solution of N-methyl-4,5,6,7-tetrahydrobenzothiophen-6-amine (7) (15.9 g, 95.06 mmol, 1 eq) in EtOAc (80 ml) and H₂O (80 mL) was added (Boc)₂O (24.89 g, 114.07 mmol, 26.20 mL, 1.2 eq) and aq. NaHCO₃ (15.97 g, 190.11 mmol, 7.40 mL, 2 eq). The mixture was stirred at 25° C. for 2 hr. The organic phase was collected. The aqueous phase was extracted with EtOAc (100 mL*3). The combined organic layers were washed with brine (100 mL), dried over anhydrous sodium sulfate, filtered and concentrated under reduced pressure to give a residue. The residue was purified by flash silica gel chromatography (ISCO®; 120 g SepaFlash® Silica Flash Column, eluent 0~6% Ethyl acetate/Petroleum ether gradient @100 mL/min). Compound tert-butyl methyl(4,5,6,7-tetrahydrobenzo[b]thiophen-6-yl)carbamate (8) (25.76 g, crude) was obtained as a yellow solid. ¹H NMR (400 MHz, CHLOROFORM-d) δ 7.08 (br d, J=4.9 Hz, 1H), 6.75 (d, J=5.0 Hz, 1H), 4.70-4.17 (m, 1H), 2.95-2.55 (m, 7H), 2.02-1.80 (m, 2H), 1.48 (s, 9H).

### Procedure for preparation of 6-[tert-butoxycarbonyl (methyl) amino]-4,5,6,7-tetrahydrobenzothiophene-2-carboxylic acid (9)

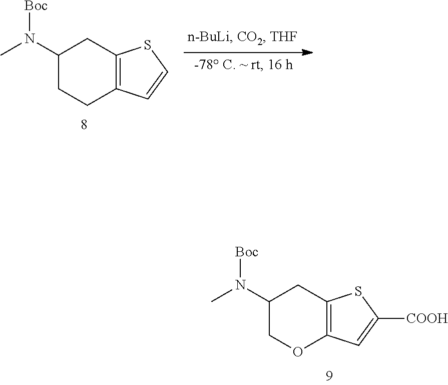 [SMILES: CN(C(=O)OC(C)(C)C)C1CCc2ccsc2C1 ; CN(C(=O)OC(C)(C)C)C1COc2cc(C(=O)O)sc2C1]

To a mixture of tert-butyl methyl(4,5,6,7-tetrahydrobenzo[b]thiophen-6-yl)carbamate (8) (10 g, 37.40 mmol, 1 eq) in THF (130 mL) was added n-BuLi (2.5 M, 16.46 mL, 1.1 eq) at −78° C. under N₂. The mixture was stirred at 78° C. for 0.5 h. Then CO₂ was bubbled into the mixture and purged with CO₂ several times. The mixture was stirred under CO₂ at −78° C. to 25° C. for 15.5 h. The reaction mixture was quenched by addition sat.NH₄Cl 150 mL at 0° C., and then diluted with H₂O 100 mL and extracted with EtOAc (100 mL*3). The combined organic layers were washed with sat.NaCl (120 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~50% Ethyl acetate/Petroleum ether gradient @40 mL/min). The compound was purified by prep-HPLC column: Kromasil Eternity XT 250*80 mm*10 μm; mobile phase: [water (NH₃H₂O)-ACN]; gradient: 0%-30% B over 20 min). Compound 6-[tert-butoxycarbonyl (methyl) amino]-4, 5, 6, 7-tetrahydrobenzothiophene-2-carboxylic acid (9) (10 g, 32.11 mmol, 85.87% yield) was obtained as a yellow solid. ¹H NMR (400 MHz, CHLOROFORM-d) δ 7.54 (s, 1H), 4.64-4.22 (m, 1H), 3.08-2.68 (m, 7H), 2.02-1.78 (m, 2H), 1.49 (s, 9H).

### Procedure for preparation of 6-[tert-butoxycarbonyl(methyl)amino]-3-iodo-4,5,6,7-tetrahydrobenzothiophene-2-carboxylic acid (10)

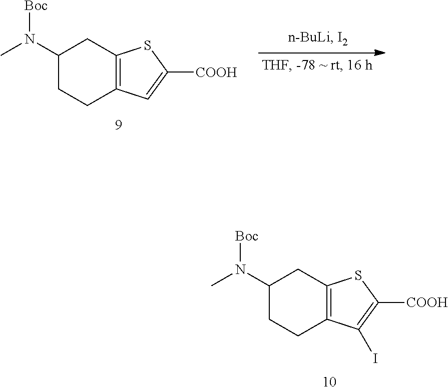 [SMILES: CN(C(=O)OC(C)(C)C)C1CCc2cc(C(=O)O)sc2C1 ; CN(C(=O)OC(C)(C)C)C1CCc2c(sc(C(=O)O)c2I)C1]

To a solution of 6-[tert-butoxycarbonyl(methyl)amino]-4,5,6,7-tetrahydrobenzothiophene-2-carboxylic acid (9) (2.2 g, 7.06 mmol, 1 eq) in THF (35 mL) was added n-BuLi (2.5 M, 6.8 mL, 2.4 eq) at −78° C. over 0.5 h. After addition, the mixture was stirred at this temperature for 0.5 hr, and then I₂ (1.79 g, 7.06 mmol, 1.42 mL, 1 eq) in THF (5 mL) was added dropwise at −78° C. The mixture was allowed to gradually warm up to 25° C. and stirred for 15 h. The reaction mixture was quenched by addition of aq. sat.NH₄Cl (60 mL) at 0° C., and then diluted with H₂O (40 mL) and extracted with EtOAc (40 mL*2). The combined organic layers were washed with aq. sat.NaCl (80 mL), dried over anhydrous Na₂SO₄, filtered and concentrated under reduced pressure to give a residue. The residue was purified by prep-HPLC (column: Phenomenex luna C18 (250*70 mm, 10 m); mobile phase: [water(FA)-ACN]; gradient:40%-70% B over 20 min). Compound 6-[tert-butoxycarbonyl(methyl)amino]-3-iodo-4,5,6,7-tetrahydrobenzothiophene-2-carboxylic acid (10) (0.5 g, 1.14 mmol, 16.18% yield) was obtained as a light yellow solid. ¹H NMR (400 MHz, METHANOL-d₄) δ 4.43-4.18 (m, 1H), 3.08-2.74 (m, 6H), 2.63-2.46 (m, 1H), 1.97 (br d, J=2.3 Hz, 2H), 1.48 (s, 9H).

### Procedure for preparation of tert-butyl N-(3-iodo-4,5,6,7-tetrahydrobenzothiophen-6-yl)-N-methyl-carbamate (11)

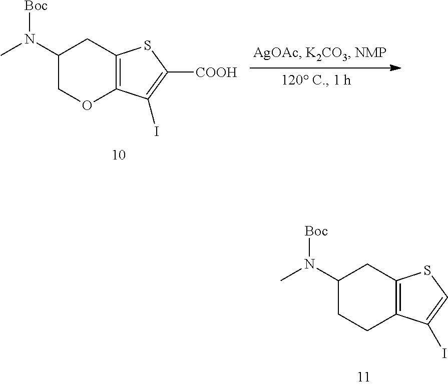 [SMILES: CN(C(=O)OC(C)(C)C)C1COc2c(sc(C(=O)O)c2I)C1 ; CN(C(=O)OC(C)(C)C)C1CCc2c(I)csc2C1]

To a solution of 6-[tert-butoxycarbonyl(methyl)amino]-3-iodo-4,5,6,7-tetrahydrobenzothiophene-2-carboxylic acid (10) (0.450 g, 1.03 mmol, 1 eq) in NMP (8 mL) was added AgOAc (42.94 mg, 257.27 μmol, 13.18 μL, 0.25 eq) and K₂CO₃ (426.67 mg, 3.09 mmol, 3 eq). The mixture was stirred at 120° C. for 1 hr. The reaction mixture was diluted with water (20 mL) and extracted with EtOAc (10 mL*3). The combined organic layers were washed with brine (10 mL*3), dried over anhydrous sodium sulfate, filtered and concentrated under reduced pressure to give a residue. The residue was purified by flash silica gel chromatography (ISCO®; 4 g SepaFlash® Silica Flash Column, eluent 0~5% Ethyl acetate/Petroleum ether gradient @24 mL/min). Compound tert-butyl N-(3-iodo-4,5,6,7-tetrahydrobenzothiophen-6-yl)-N-methyl-carbamate (11) (0.27 g, 686.53 μmol, 66.71% yield) was obtained as a white solid. ¹H NMR (400 MHz, METHANOL-d₄) δ 7.37 (s, 1H), 4.39-4.15 (m, 1H), 3.02-2.78 (m, 5H), 2.76-2.66 (m, 1H), 2.59-2.45 (m, 1H), 2.00-1.90 (m, 2H), 1.47 (s, 9H).

### Procedure for preparation of tert-butyl N-methyl-N-[3-(trifluoromethyl)-4,5,6,7-tetrahydrobenzothiophen-6-yl]carbamate (12)

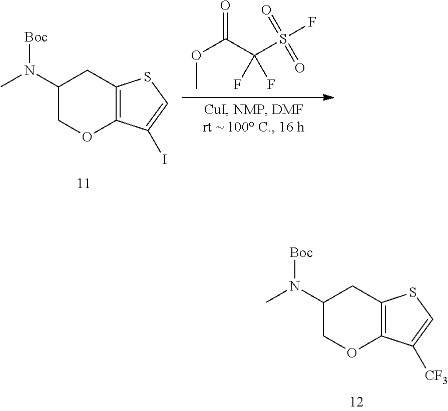

To a solution of tert-butyl N-(3-iodo-4,5,6,7-tetrahydrobenzothiophen-6-yl)-N-methyl-carbamate (11) (0.22 g, 559.39 μmol, 1 eq) in DMF (6 mL) was added CuI (532.68 mg, 2.80 mmol, 5 eq) and NMP (83.18 mg, 839.09 μmol, 81.55 μL, 1.5 eq), methyl 2,2-difluoro-2-fluorosulfonyl-acetate (537.34 mg, 2.80 mmol, 355.85 μL, 5 eq). The mixture was stirred at 100° C. for 16 hr. The reaction mixture was poured into H₂O (10 mL). The reaction was diluted with NH₃·H₂O 5 mL and extracted with EtOAc (5 mL*3). The combined organic layers were washed with brine (5 mL*3), dried over anhydrous sodium sulfate, filtered and concentrated under reduced pressure to give a residue. The residue was purified by flash silica gel chromatography (ISCO®; 12 SepaFlash® Silica Flash Column, eluent 0-25% Ethyl acetate/Petroleum ether gradient @24 mL/min). Compound tert-butyl N-methyl-N-[3-(trifluoromethyl)-4,5,6,7-tetrahydrobenzothiophen-6-yl]carbamate (12) (0.08 g) was obtained as a yellow oil. ¹H NMR (400 MHz, CHLOROFORM-d) δ 7.50 (s, 1H), 4.60-4.10 (m, 1H), 3.00-2.69 (m, 7H), 2.02-1.82 (m, 2H), 1.48 (s, 9H).

### Procedure for preparation of N-methyl-3-(trifluoromethyl)-4,5,6,7-tetrahydrobenzothiophen-6-amine hydrochloride (180)

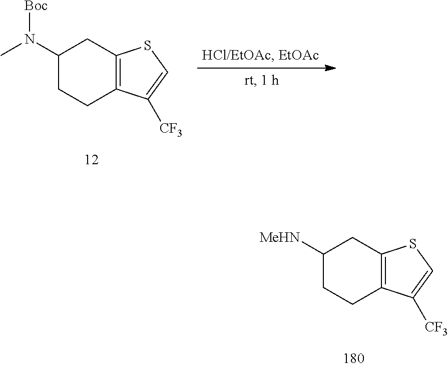 [SMILES: CN(C(=O)OC(C)(C)C)C1CCc2c(C(F)(F)F)csc2C1 ; CNC1CCc2c(C(F)(F)F)csc2C1]

To a solution of tert-butyl N-methyl-N-[3-(trifluoromethyl)-4,5,6,7-tetrahydrobenzothiophen-6-yl]carbamate (12) (0.08 g, 238.81 μmol, 1 eq) in EtOAc (2 mL) was added HCl/EtOAc (4 M, 2 mL). The mixture was stirred at 25° C. for 1 hr. The reaction mixture was concentrated under reduced pressure to give N-methyl-3-(trifluoromethyl)-4,5,6,7-tetrahydrobenzothiophen-6-amine hydrochloride (180) (57.23 mg, 233.52 μmol, 30.12% yield) as a white solid. ¹H NMR (400 MHz, METHANOL-d₄) δ 7.86 (s, 1H), 3.65-3.54 (m, 1H), 3.44-3.36 (m, 1H), 3.00-2.77 (m, 6H), 2.39-2.26 (m, 1H), 2.03-1.90 (m, 1H). LCMS: m/z [M+H]⁺=236.2.

### Synthetic scheme for preparation of N, 2-dimethyl-6,7-dihydro-5H-thieno[3,2-b]pyran-6-amine hydrochloride (181)

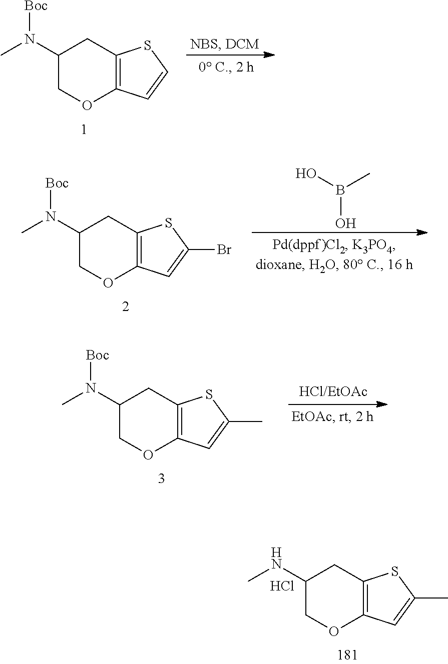

### Procedure for preparation of tert-butyl N-(2-bromo-6,7-dihydro-5H-thieno[3,2-b]pyran-6-yl)-N-methyl-carbamate (2)

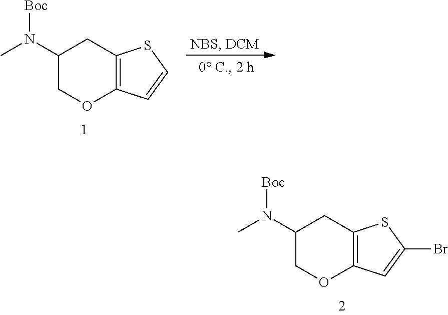

To a solution of tert-butyl N-(6,7-dihydro-5H-thieno[3,2-b]pyran-6-yl)-N-methyl-carbamate (1) (500 mg, 1.86 mmol, 1 eq) in DCM (5 mL) was added NBS (396.46 mg, 2.23 mmol, 1.2 eq) at 0° C. The mixture was stirred at 0° C. for 2 hr. The reaction mixture was quenched by addition aq. Na₂SO₃ (10 mL) and aq. sat. NaHCO₃ (10 ml) and extracted with EtOAc (15 mL*2). The combined organic layers were washed with aq. sat. NaCl (10 mL), dried over Na₂SO₄, filtered and concentrated under reduced pressure to give a residue. The residue was purified by column chromatography (SiO₂, Petroleum ether/Ethyl acetate=5/1). Compound tert-butyl N-(2-bromo-6,7-dihydro-5H-thieno[3,2-b]pyran-6-yl)-N-methyl-carbamate (2) (530 mg, 1.52 mmol, 81.99% yield) was obtained as a white solid. LCMS: m/z [M+H-tBu]⁺=294.1

### Procedure for preparation of tert-butyl N-methyl-N-(2-methyl-6,7-dihydro-5H-thieno[3,2-b]pyran-6-yl)carbamate (3)

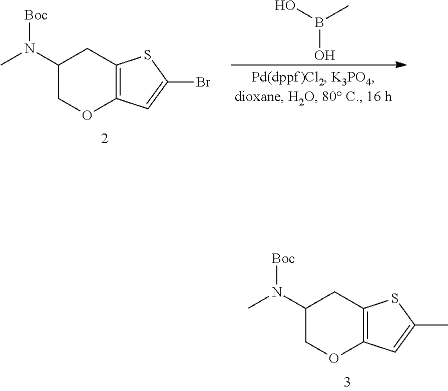

A mixture of tert-butyl N-(2-bromo-6,7-dihydro-5H-thieno[3,2-b]pyran-6-yl)-N-methyl-carbamate (2) (530 mg, 1.52 mmol, 1 eq), methylboronic acid (455.50 mg, 7.61 mmol, 5 eq), Pd(dppf)Cl₂ (55.68 mg, 76.09 μmol, 0.05 eq) and tripotassium phosphate (969.13 mg, 4.57 mmol, 3 eq) in dioxane (10 mL) and H₂O (2 mL) was degassed and purged with N₂ for 3 times, and then the mixture was stirred at 80° C. for 16 hr under N₂ atmosphere. The reaction mixture was then concentrated under reduced pressure to remove dioxane. Diluted with water (10 mL), extracted with EtOAc (10 mL*2). The combined organic layers were washed with aq. sat. NaCl (10 mL), dried over Na₂SO₄, filtered and concentrated under reduced pressure to give a residue. The residue was purified by prep-HPLC (neutral condition:column: Waters Xbridge 150*25 mm*5 μm; mobile phase: [water (ammonia hydroxide v/v)-ACN]; gradient:52%-82% B over min). Compound tert-butyl N-methyl-N-(2-methyl-6,7-dihydro-5H-thieno[3,2-b]pyran-6-yl)carbamate (3) (60 mg, 141.15 μmol, 9.27% yield) was obtained as a white solid. ¹H NMR (400 MHz, DMSO-d₆) δ=6.36 (d, J=1.0 Hz, 1H), 4.41-4.23 (m, 1H), 4.06-4.03 (m, 2H), 2.92-2.81 (m, 1H), 2.80-2.70 (m, 4H), 2.32 (s, 3H), 1.41 (s, 9H).

### Procedure for preparation of N, 2-dimethyl-6,7-dihydro-5H-thieno[3,2-b]pyran-6-amine hydrochloride (181)

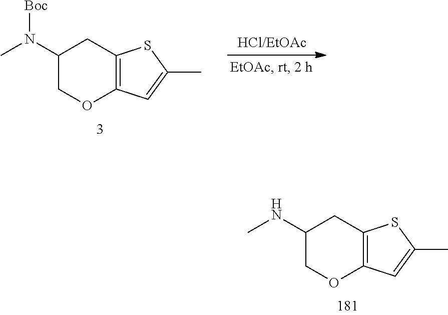

To a solution of tert-butyl N-methyl-N-(2-methyl-6,7-dihydro-5H-thieno[3,2-b]pyran-6-yl)carbamate (3) (40 mg, 141.15 μmol, 1 eq) in EtOAc (1 mL) was added HCl/EtOAc (4 M, 3.00 mL, 56.68 eq). The mixture was stirred at 25° C. for 2 hr. The reaction mixture was concentrated under reduced pressure to remove EtOAc. The crude product was triturated with EtOAc (1 mL) at 25° C. for 30 min. Compound N, 2-dimethyl-6,7-dihydro-5H-thieno[3,2-b]pyran-6-amine hydrochloride (181) (40.30 mg, 183.41 μmol, 86.62% yield) was obtained as a brown solid. ¹H NMR (400 MHz, METHANOL-d₄) 6=6.37 (s, 1H), 4.51-4.44 (m, 1H), 4.19 (d, J=12.3 Hz, 1H), 3.81 (br s, 1H), 3.24 (dd, J=5.6, 17.3 Hz, 1H), 2.96 (br d, J=17.3 Hz, 1H), 2.81 (s, 3H), 2.37 (s, 3H). LCMS: m/z [M+H]⁺=184.1

### Synthetic scheme for preparation of 3-fluoro-N, 5-dimethyl-6,7-dihydro-4H-benzothiophen-5-amine hydrochloride (182)

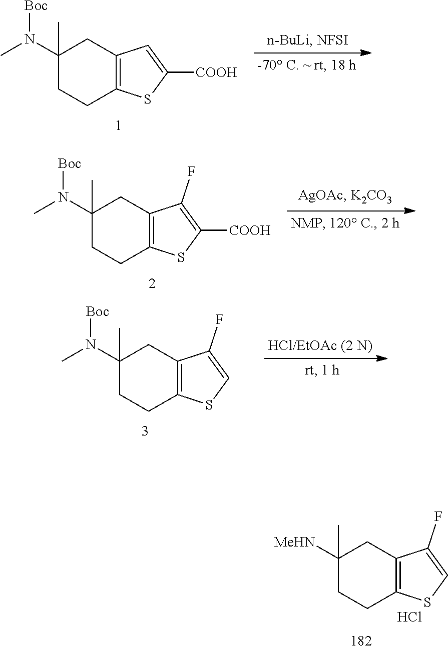 [SMILES: CN(C(=O)OC(C)(C)C)C1(C)CCc2sc(C(=O)O)cc2C1 ; CN(C(=O)OC(C)(C)C)C1(C)CCc2sc(C(=O)O)c(F)c2C1 ; CN(C(=O)OC(C)(C)C)C1(C)CCc2scc(F)c2C1 ; CNC1(C)CCc2scc(F)c2C1 ; Cl]

### Procedure for preparation of 5-[tert-butoxycarbonyl(methyl)amino]-3-fluoro-5-methyl-6,7-dihydro-4H-benzothiophene-2-carboxylic acid (2)

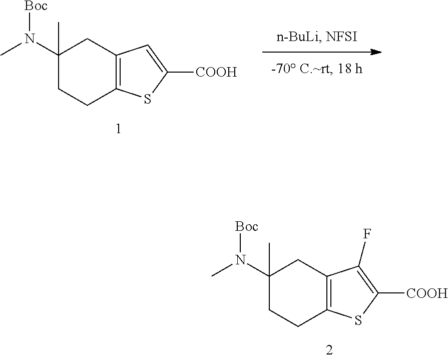 [SMILES: CN(C(=O)OC(C)(C)C)C1(C)CCc2sc(C(=O)O)cc2C1 ; CN(C(=O)OC(C)(C)C)C1(C)CCc2sc(C(=O)O)c(F)c2C1]

To the solution of 5-[tert-butoxycarbonyl(methyl)amino]-5-methyl-6,7-dihydro-4H-benzothiophene-2-carboxylic acid (1) (0.5 g, 1.54 mmol, 1.00 eq) in THF (10 mL) was added n-BuLi (2.5 M, 1.50 mL, 2.40 eq) at −70° C. under nitrogen atmosphere. After stirring for 1 h, a solution of NFSI (630 mg, 2.00 mmol, 1.30 eq) in THF (5 mL) was added drop-wise at −70° C. and the resulting mixture was stirred for 1 h at −70° C. Then the mixture was warmed to room temperature slowly and stirred for 16 h. The reaction mixture was added to water (15 mL) and aq. sat. NH₄Cl (15 mL) slowly, adjusted pH to 3-4 with sat. citric acid and extracted with EtOAc (10 mL*3). The combined organic layers were washed with brine (15 mL), dried over anhydrous sodium sulfate, filtered and concentrated under vacuum to give 5-[tert-butoxycarbonyl(methyl)amino]-3-fluoro-5-methyl-6,7-dihydro-4H-benzothiophene-2-carboxylic acid (2) (500 mg, crude) as a yellow solid which was used into next step without further purification. LCMS: m/z [M+H-tBu]⁺=228.0.

### Procedure for preparation of tert-butyl N-(3-fluoro-5-methyl-6,7-dihydro-4H-benzothiophen-5-yl)-N-methyl-carbamate (3)

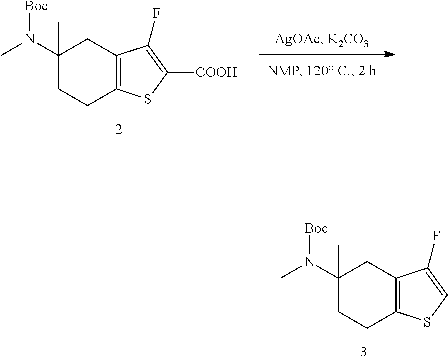 [SMILES: CN(C(=O)OC(C)(C)C)C1(C)CCc2sc(C(=O)O)c(F)c2C1 ; CN(C(=O)OC(C)(C)C)C1(C)CCc2scc(F)c2C1]

To the mixture of 5-[tert-butoxycarbonyl(methyl)amino]-3-fluoro-5-methyl-6,7-dihydro-4H-benzothiophene-2-carboxylic acid (2) (300 mg, 874 μmol, 1.00 eq) in NMP (2 mL) was added AgOAc (29 mg, 175 μmol, 0.20 eq) and K₂CO₃ (362 mg, 2.62 mmol, 3.00 eq). The resulting mixture was stirred at 120° C. for 2 h. The reaction mixture was diluted with EtOAc (10 mL) and filtered. The filtrate was concentrated under vacuum to give a residue. The residue was purified by flash silica gel chromatography (ISCO®; 4 g SepaFlash® Silica Flash Column, eluent 0~5% Ethyl acetate/Petroleum ether gradient @18 mL/min) to give tert-butyl N-(3-fluoro-5-methyl-6,7-dihydro-4H-benzothiophen-5-yl)-N-methyl-carbamate (3) (70 mg, 234 μmol, 26.7% yield) as a white solid. ¹H NMR (400 MHz, CDCl₃-d) 6=6.47 (s, 1H), 3.05 (d, J=16.4 Hz, 1H), 2.97-2.87 (m, 1H), 2.77 (s, 3H), 2.70 (t, J=6.4 Hz, 2H), 2.51 (d, J=16.8 Hz, 1H), 1.80 (td, J=8.0, 12.8 Hz, 1H), 1.47 (s, 9H), 1.43 (s, 3H). ¹⁹F NMR (400 MHz, CDCl₃-d) 6=132.97. LCMS: m/z [M+H-Boc]⁺=200.0.

### Procedure for preparation of 3-fluoro-N, 5-dimethyl-6,7-dihydro-4H-benzothiophen-5-amine hydrochloride (182)

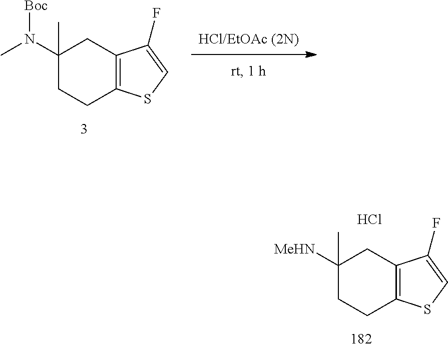 [SMILES: CN(C(=O)OC(C)(C)C)C1(C)CCc2scc(F)c2C1 ; Cl ; CNC1(C)CCc2scc(F)c2C1]

To the solution of tert-butyl N-(3-fluoro-5-methyl-6,7-dihydro-4H-benzothiophen-5-yl)-N-methyl-carbamate (3) (100 mg, 334.00 μmol, 1.00 eq) in EtOAc (2 mL) was added HCl/EtOAc (4 M, 2 mL). The resulting mixture was stirred at room temperature for 1 h. The reaction mixture was filtered. The cake was washed with EtOAc (2 mL) and dried over vacuum to give 3-fluoro-N, 5-dimethyl-6,7-dihydro-4H-benzothiophen-5-amine (182) (51.24 mg, 211.59 μmol, 63.35% yield) as a white solid. ¹H NMR (400 MHz, MeOD-d₄) δ=6.78 (s, 1H), 2.91 (t, J=6.4 Hz, 2H), 2.84-2.75 (m, 2H), 2.73 (s, 3H), 2.10 (t, J=6.4 Hz, 2H), 1.41 (s, 3H). ¹⁹F NMR (400 MHz, CDCl₃-d) 6=134.99. LCMS: m/z [M+H]⁺=200.0.

### Synthetic Scheme for preparation of 2-cyclopropyl-N-methyl-4,5,6,7-tetrahydrobenzothiophen-5-amine hydrochloride enantiomer 1 (183) and enantiomer 2 (184)

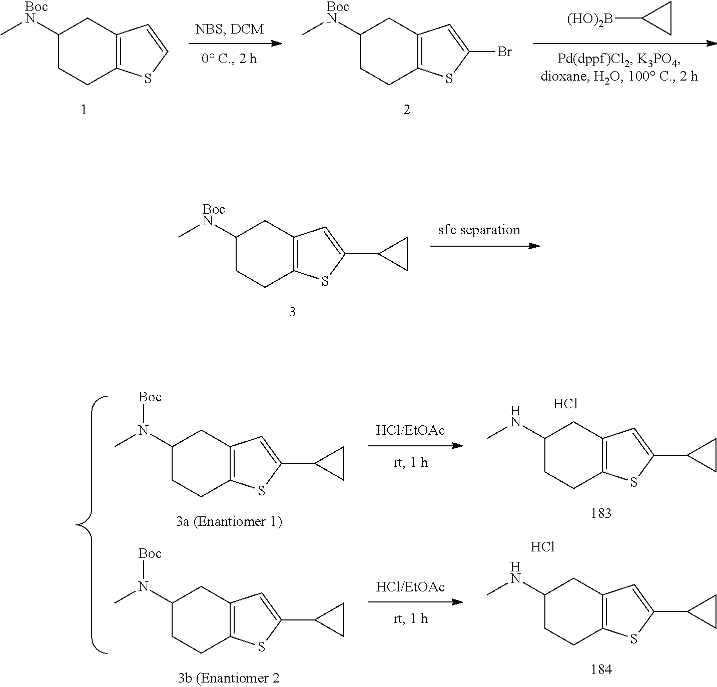

### Procedure for preparation of tert-butyl N-(2-bromo-4,5,6,7-tetrahydrobenzothiophen-5-yl)-N-methyl-carbamate (2)

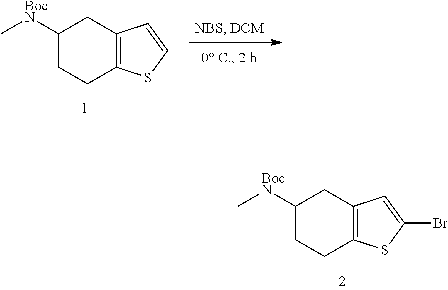 [SMILES: CN(C(=O)OC(C)(C)C)C1CCc2sccc2C1 ; CN(C(=O)OC(C)(C)C)C1CCc2sc(Br)cc2C1]

To a solution of tert-butyl N-methyl-N-(4,5,6,7-tetrahydrobenzothiophen-5-yl)carbamate (1) (1 g, 3.74 mmol, 1 eq) in DCM (10 mL) was added NBS (798 mg, 4.49 mmol, 1.2 eq) at 0° C. The mixture was stirred at 0° C. for 2 hr. The reaction mixture was concentrated and diluted with H₂O (20 mL) and extracted with EtOAc (20 mL*2). The combined organic layers were washed with aq. sat. NaCl (10 mL), dried over anhydrous Na₂SO₄, filtered and concentrated under reduced pressure to give a residue. The residue was purified by flash silica gel chromatography (ISCO; 12 g SepaFlash Silica Flash Column, eluent 0~12% Ethyl acetate/Petroleum ether gradient @40 mL/min). Compound tert-butyl N-(2-bromo-4,5,6,7-tetrahydrobenzothiophen-5-yl)-N-methyl-carbamate (2) (1 g, 2.89 mmol, 77.22% yield) was obtained as a yellow oil. ¹H NMR (400 MHz, CHLOROFORM-d) δ=6.69 (s, 1H), 4.53-4.18 (m, 1H), 2.84-2.78 (m, 5H), 2.70-2.62 (m, 2H), 1.99-1.84 (m, 2H), 1.48 (s, 9H)

### Procedure for preparation of tert-butyl N-(2-cyclopropyl-4,5,6,7-tetrahydrobenzothiophen-5-yl)-N-methyl-carbamate (3)

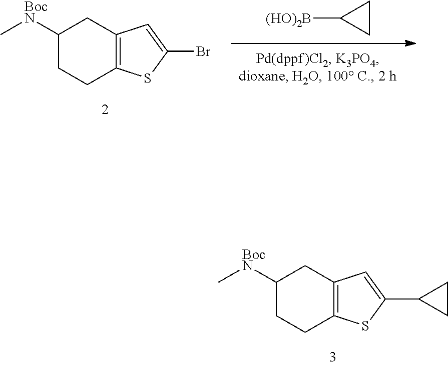 [SMILES: CN(C(=O)OC(C)(C)C)C1CCc2sc(Br)cc2C1 ; OB(O)C1CC1 ; CN(C(=O)OC(C)(C)C)C1CCc2sc(C3CC3)cc2C1]

A mixture of tert-butyl N-(2-bromo-4,5,6,7-tetrahydrobenzothiophen-5-yl)-N-methyl-carbamate (2) (900 mg, 2.60 mmol, 1 eq), cyclopropylboronic acid (334 mg, 3.90 mmol, 1.5 eq), Pd(dppf)Cl₂ (95.1 mg, 129 μmol, 0.05 eq), K₃PO₄ (1.66 g, 7.80 mmol, 3 eq) in dioxane (10 mL) and H₂O (1 mL) was degassed and purged with N₂ for 3 times, and then the mixture was stirred at 100° C. for 2 hr under N₂ atmosphere. The reaction mixture was diluted with H₂O (10 mL) and extracted with EtOAc (30 mL*2). The combined organic layers were washed with aq. sat. NaCl (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~15% Ethyl acetate/Petroleum ether gradient @40 mL/min) and then by prep-HPLC (column: YMC Triart C18 150*25 mm*5 μm; mobile phase: [water(FA)-ACN]; gradient:78%-98% B over 10 min). Compound tert-butyl N-(2-cyclopropyl-4,5,6,7-tetrahydrobenzothiophen-5-yl)-N-methyl-carbamate (3) (200 mg, 650.51 μmol, 25.03% yield) was obtained as a white solid. LCMS: m/z [M+H-tBu]⁺=252.1

### Procedure for preparation of tert-butyl N-(2-cyclopropyl-4,5,6,7-tetrahydrobenzothiophen-5-yl)-N-methyl-carbamate enantiomer 1 (3a) and enantiomer 2 (3b)

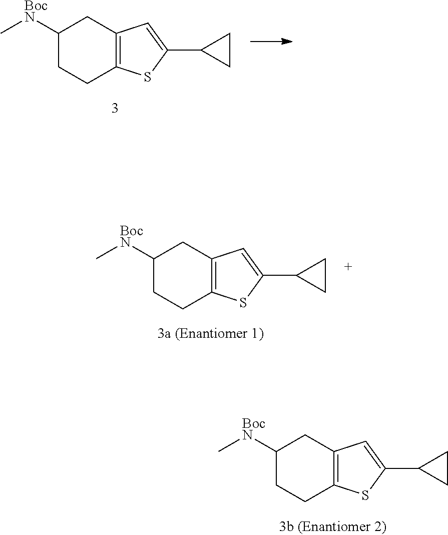 [SMILES: CN(C(=O)OC(C)(C)C)C1CCc2sc(C3CC3)cc2C1 ; CN(C(=O)OC(C)(C)C)C1CCc2sc(C3CC3)cc2C1 ; CN(C(=O)OC(C)(C)C)C1CCc2sc(C3CC3)cc2C1]

Racemic tert-butyl N-(2-cyclopropyl-4,5,6,7-tetrahydrobenzothiophen-5-yl)-N-methyl-carbamate (3) was separated by SFC (column: DAICEL CHIRALPAK IG (250 mm*50 mm, 10 μm); mobile phase: [CO₂-MeOH (0.1% NH₃H₂O)]; B %:10%, isocratic elution mode) to afford enantiomers. The 1st enantiomer 3a (RT=0.712 min, 70 mg) and the 2nd enantiomer 3b (RT=0.778 min, 80 mg), both isolated as white solids.

### Procedure for preparation of 2-cyclopropyl-N-methyl-4,5,6,7-tetrahydrobenzothiophen-5-amine hydrochloride enantiomer 1 (183)

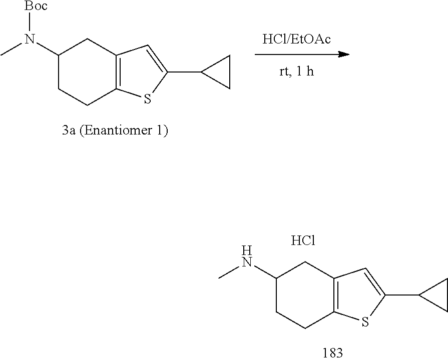 [SMILES: CN(C(=O)OC(C)(C)C)C1CCc2sc(C3CC3)cc2C1 ; Cl ; CNC1CCc2sc(C3CC3)cc2C1]

To a solution of tert-butyl N-(2-cyclopropyl-4,5,6,7-tetrahydrobenzothiophen-5-yl)-N-methyl-carbamate enantiomer 1 (3a) (70 mg, 227 μmol, 1 eq) in EtOAc (2 mL) was added HCl/EtOAc (4 M, 2 mL, 35.1 eq). The mixture was stirred at 25° C. for 2 hr. The reaction mixture was filtered and the cake was washed with EtOAc (10 mL). Compound 2-cyclopropyl-N-methyl-4,5,6,7-tetrahydrobenzothiophen-5-amine hydrochloride enantiomer 1 (183) (46.25 mg, 187.05 μmol, 82.16% yield) was obtained as a white solid. ¹H NMR (400 MHz, METHANOL-d4) δ=6.48 (s, 1H), 3.54-3.43 (m, 1H), 3.08 (dd, J=4.9, 16.1 Hz, 1H), 2.92-2.84 (m, 2H), 2.79 (s, 3H), 2.68-2.58 (m, 1H), 2.36-2.25 (m, 1H), 2.06-1.87 (m, 2H), 1.01-0.91 (m, 2H), 0.68-0.59 (m, 2H). LCMS: m/z [M+H]⁺=208.1

### Procedure for preparation of 3-cyclopropyl-N-methyl-4,5,6,7-tetrahydro-2-benzothiophen-5-amine hydrochloride enantiomer 2 (184)

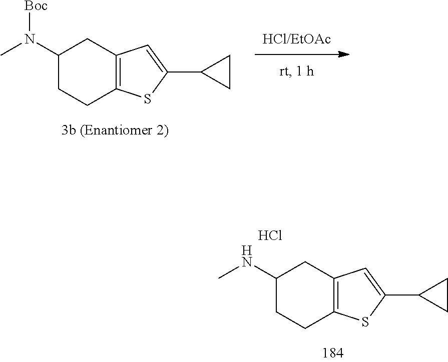 [SMILES: CN(C(=O)OC(C)(C)C)C1CCc2sc(C3CC3)cc2C1 ; Cl ; CNC1CCc2sc(C3CC3)cc2C1]

To a solution of tert-butyl N-(2-cyclopropyl-4,5,6,7-tetrahydrobenzothiophen-5-yl)-N-methyl-carbamate enantiomer 2 (3b) (80 mg, 260.20 μmol, 1 eq) in EtOAc (2 mL) was added HCl/EtOAc (4 M, 2 mL, 30.74 eq). The mixture was stirred at 25° C. for 1 hr. The reaction mixture was filtered and the cake was washed with EtOAc (10 mL). Compound 2-cyclopropyl-N-methyl-4,5,6,7-tetrahydrobenzothiophen-5-amine hydrochloride enantiomer 1 (184) (56.76 mg, 227.93 μmol, 87.60% yield) was obtained as a white solid. ¹H NMR (400 MHz, METHANOL-d4) δ=6.37 (s, 1H), 3.40-3.33 (m, 1H), 2.96 (dd, J=5.3, 15.6 Hz, 1H), 2.80-2.72 (m, 2H), 2.67 (s, 3H), 2.57-2.46 (m, 1H), 2.24-2.13 (m, 1H), 1.93-1.77 (m, 2H), 0.89-0.81 (m, 2H), 0.56-0.48 (m, 2H). LCMS: m/z [M+H]⁺=208.1

### Synthetic scheme for preparation of 2-methyl-4,5,6,7-tetrahydrobenzothiophen-5-amine hydrochloride (185)

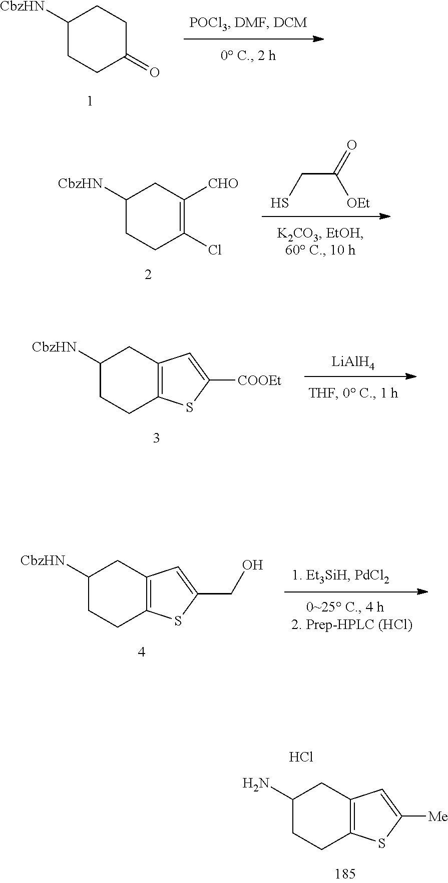

### Procedure for preparation of benzyl N-(4-chloro-3-formyl-cyclohex-3-en-1-yl)carbamate (2)

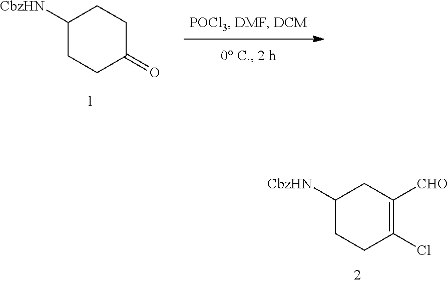 [SMILES: O=C1CCC(NC(=O)OCc2ccccc2)CC1 ; O=CC1=C(Cl)CCC(NC(=O)OCc2ccccc2)C1]

To a solution of DMF (2.66 g, 36.39 mmol, 2.80 mL, 4.5 eq) in DCM (10 mL) was added POCl₃ (4.96 g, 32.35 mmol, 3.02 mL, 4 eq) in DCM (5 mL) at 0° C., the reaction was stirred at 20 for 1 h, then was added benzyl N-(4-oxocyclohexyl)carbamate (1) (2 g, 8.09 mmol, 1 eq) in DCM (5 mL). The mixture was stirred at 0° C. for 2 hr. The reaction mixture was quenched by addition aq. sat. NaHCO₃ 300 mL at 0° C., and extracted with EtOAc (150 mL*2). The combined organic layers were washed with brine (100 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~25% Ethyl acetate/Petroleum ether gradient @40 mL/min). Compound benzyl N-(4-chloro-3-formyl-cyclohex-3-en-1-yl)carbamate (2) (2 g, 6.81 mmol, 84.18% yield) was obtained as a yellow oil. LCMS: m/z [M+H]⁺=293.8. 1H NMR (400 MHz, CHLOROFORM-d) δ=10.16 (s, 1H), 7.46-7.30 (m, 5H), 5.11 (s, 2H), 4.75 (br s, 1H), 3.91 (br s, 1H), 2.88-2.62 (m, 3H), 2.20-2.05 (m, 2H), 1.88-1.71 (m, 1H)

### Procedure for preparation of ethyl 5-(benzyloxycarbonylamino)-4,5,6,7-tetrahydrobenzothiophene-2-carboxylate (3)

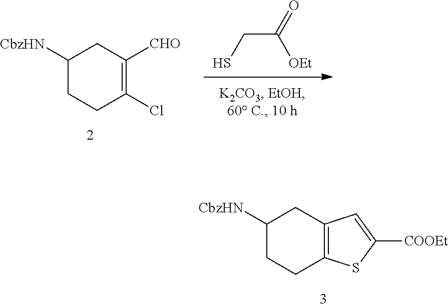 [SMILES: O=CC1=C(Cl)CCC(NC(=O)OCc2ccccc2)C1 ; CCOC(=O)CS ; CCOC(=O)c1cc2c(s1)CCC(NC(=O)OCc1ccccc1)C2]

To a solution of benzyl N-(4-chloro-3-formyl-cyclohex-3-en-1-yl)carbamate (2) (1 g, 3.40 mmol, 1 eq) and K₂CO₃ (705.74 mg, 5.11 mmol, 1.5 eq) in EtOH (15 mL) was added ethyl 2-sulfanylacetate (630 mg, 5.24 mmol, 574.82 μL, 1.54 eq). The mixture was stirred at 60° C. for 10 hr. The reaction mixture was concentrated under reduced pressure to remove EtOH. The residue was diluted with H₂O (20 mL) and extracted with EtOAc (20 mL*2). The combined organic layers were washed with aq. sat.NaCl (20 mL), dried over anhydrous Na₂SO₄, filtered and concentrated under reduced pressure to give a residue. The residue was purified by flash silica gel chromatography (ISCO®; 20 g SepaFlash® Silica Flash Column, eluent 0~20% Ethyl acetate/Petroleum ether gradient @35 mL/min). Compound ethyl 5-(benzyloxycarbonylamino)-4,5,6,7-tetrahydrobenzothiophene-2-carboxylate (3) (1.1 g, 3.06 mmol, 89.90% yield) was obtained as a yellow oil. LCMS: m/z [M+H]⁺=360.1. ¹H NMR (400 MHz, CHLOROFORM-d) δ=7.45 (s, 1H), 7.40-7.33 (m, 5H), 5.17-5.08 (m, 2H), 4.87-4.75 (m, 1H), 4.40-4.28 (m, 2H), 4.12-4.05 (m, 1H), 3.08-2.99 (m, 1H), 2.96-2.87 (m, 2H), 2.57-2.49 (m, 1H), 2.17-2.08 (m, 1H), 1.96-1.86 (m, 1H), 1.40-1.32 (m, 3H)

### Procedure for preparation of benzyl N-[2-(hydroxymethyl)-4,5,6,7-tetrahydrobenzothiophen-5-yl]carbamate (4)

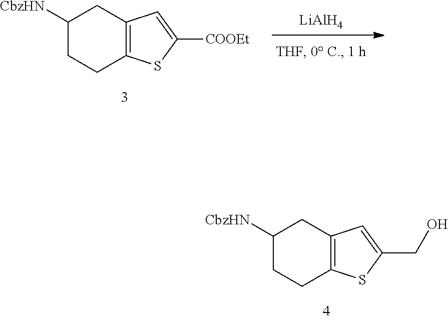 [SMILES: CCOC(=O)c1cc2c(s1)CCC(NC(=O)OCc1ccccc1)C2 ; "LiAlH4" ; O=C(NC1CCc2sc(CO)cc2C1)OCc1ccccc1]

To a solution of ethyl 5-(benzyloxycarbonylamino)-4,5,6,7-tetrahydrobenzothiophene-2-carboxylate (3) (480 mg, 1.34 mmol, 1 eq) in THF (4 mL) was added LiAlH₄ (101.37 mg, 2.67 mmol, 2 eq) at 0° C. The mixture was stirred at 0° C. for 1 hr. The mixture was quenched with Na₂SO₄·10H₂O (1 g). The mixture was diluted with EtOAc (50 mL) and filtered. The filtrate was diluted with H₂O (50 mL) and extracted with EtOAc (50 mL*3). The combined organic phases were washed with brine (50 mL*3), dried over Na₂SO₄, filtered and concentrated at reduced pressure to give a residue. The residue was purified by flash silica gel chromatography (ISCO®; 12 g SepaFlash® Silica Flash Column, eluent 0~35% Ethyl acetate/Petroleum ether gradient @35 mL/min). Compound benzyl N-[2-(hydroxymethyl)-4,5,6,7-tetrahydrobenzothiophen-5-yl]carbamate (4) (400 mg, 1.26 mmol, 94.37% yield) was obtained as a white solid. LCMS: m/z [M-OH]⁺=300.0.

### Procedure for preparation of 2-methyl-4,5,6,7-tetrahydrobenzothiophen-5-amine hydrochloride (185)

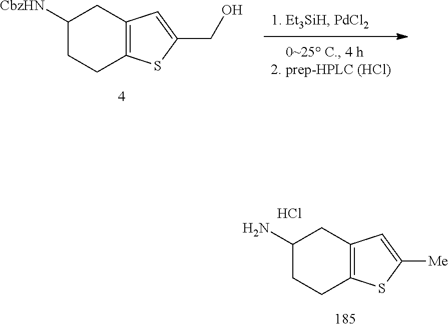 [SMILES: O=C(NC1CCc2sc(CO)cc2C1)OCc1ccccc1 ; Cl ; Cc1cc2c(s1)CCC(N)C2]

To a solution of benzyl N-[2-(hydroxymethyl)-4,5,6,7-tetrahydrobenzothiophen-5-yl]carbamate (4) (340 mg, 1.07 mmol, 1 eq) and PdCl₂ (19.00 mg, 107.12 μmol, 0.1 eq) in EtOH (10 mL) was added triethylsilane (249.11 mg, 2.14 mmol, 342.19 μL, 2 eq) at 0° C. The mixture was stirred at 25° C. for 4 hr. The reaction mixture was partitioned between EtOAc (50 mL) and H₂O (50 mL). The organic phase was separated, washed with brine (50 mL*3), dried over Na₂SO₄, filtered and concentrated under reduced pressure to give a residue. The residue was purified by prep-HPLC (column: Phenomenex luna C18 150*25 mm*10 μm; mobile phase: [water(HCl)-ACN]; gradient:1%-25% B over 10 min). Compound 2-methyl-4,5,6,7-tetrahydrobenzothiophen-5-amine hydrochloride (185) (13.81 mg, 67.79 μmol, 6.33% yield) was obtained as a white solid. LCMS: m/z [M-NH₂]⁺=151.0. ¹H NMR (400 MHz, METHANOL-d4) δ=6.44 (s, 1H), 3.59-3.49 (m, 1H), 2.99 (br dd, J=5.0, 15.8 Hz, 1H), 2.89-2.83 (m, 2H), 2.59 (br dd, J=9.2, 15.6 Hz, 1H), 2.38 (s, 3H), 2.22 (br dd, J=4.3, 8.0 Hz, 1H), 1.97-1.86 (m, 1H)

### Synthetic scheme for synthesis 3-(trifluoromethyl)-4,5,6,7-tetrahydrobenzothiophen-5-amine hydrochloride (186)

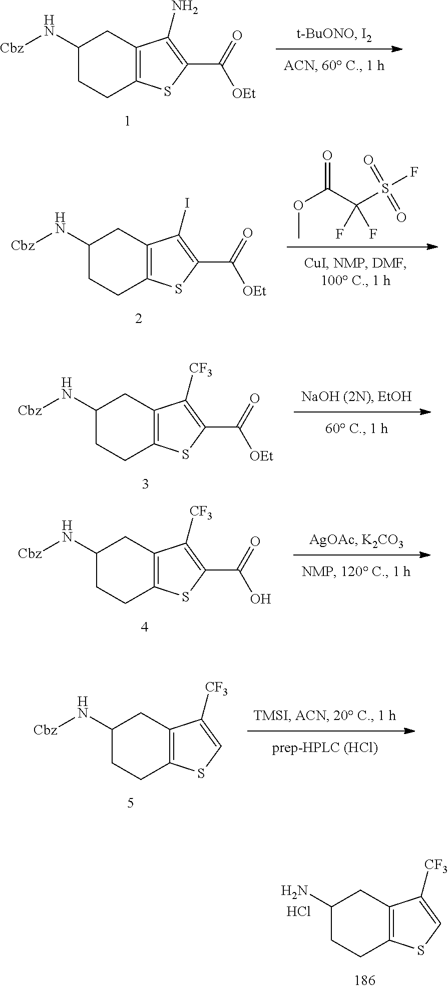

### Procedure for preparation of ethyl 5-(benzyloxycarbonylamino)-3-iodo-4,5,6,7-tetrahydrobenzo[b]thiophene-2-carboxylate (2)

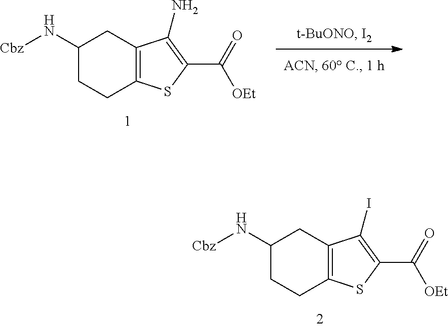 [SMILES: CCOC(=O)c1sc2c(c1N)CC(NC(=O)OCc1ccccc1)CC2 ; CCOC(=O)c1sc2c(c1I)CC(NC(=O)OCc1ccccc1)CC2]

To a solution of ethyl 3-amino-5-(benzyloxycarbonylamino)-4,5,6,7-tetrahydrobenzothiophene-2-carboxylate (1) (5 g, 13.35 mmol, 1 eq) in ACN (100 mL) was added t-BuONO (2. g, 20.03 mmol, 2.4 mL, 1.5 eq) and 12 (10.17 g, 40.06 mmol, 8.07 mL, 3 eq). The mixture was stirred at 60° C. for 1 hr. The reaction mixture was concentrated under reduced pressure to give a residue. The residue was purified by flash silica gel chromatography (ISCO®; 40 g SepaFlash® Silica Flash Column, eluent 0~25% Ethyl acetate/Petroleum ether gradient @40 mL/min) to give ethyl 5-(benzyloxycarbonylamino)-3-iodo-4,5,6,7-tetrahydrobenzothiophene-2-carboxylate (2) (5 g, 8.86 mmol, 66.35% yield) as a brown solid. ¹H NMR (400 MHz, CHLOROFORM-d) δ=7.44-7.31 (m, 5H), 5.13 (s, 2H), 4.85 (br d, J=6.8 Hz, 1H), 4.36 (q, J=7.1 Hz, 2H), 4.11 (br s, 1H), 3.02-2.85 (m, 3H), 2.41 (dd, J=7.6, 16.3 Hz, 1H), 2.18-2.07 (m, 1H), 1.98-1.80 (m, 1H), 1.38 (t, J=7.1 Hz, 3H). LCMS: m/z [M+H]⁺=485.9

### Procedure for preparation of ethyl 5-(benzyloxycarbonylamino)-3-(trifluoromethyl)-4,5,6,7-tetrahydrobenzothiophene-2-carboxylate (3)

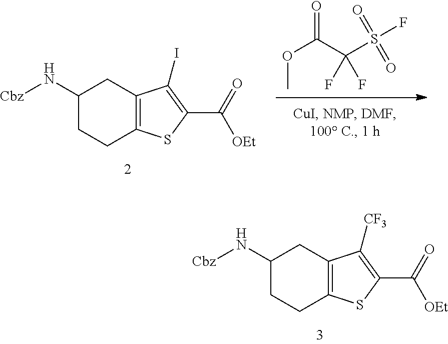 [SMILES: CCOC(=O)c1sc2c(c1I)CC(NC(=O)OCc1ccccc1)CC2 ; COC(=O)C(F)(F)S(=O)(=O)F ; CCOC(=O)c1sc2c(c1C(F)(F)F)CC(NC(=O)OCc1ccccc1)CC2]

To a solution of ethyl 5-(benzyloxycarbonylamino)-3-iodo-4,5,6,7-tetrahydrobenzothiophene-2-carboxylate (2) (3 g, 5.32 mmol, 1 eq) and methyl 2,2-difluoro-2-fluorosulfonyl-acetate (5.11 g, 26.58 mmol, 3.38 mL, 5 eq) in DMF (50 mL) was added CuI (1.11 g, 5.85 mmol, 1.1 eq) and NMP (2.63 g, 26.58 mmol, 2.58 mL, 5 eq). The mixture was stirred at 100° C. for 1 hr under N₂. The reaction mixture was diluted with H₂O 100 mL and extracted with EtOAc (40 mL*2). The combined organic layers were washed with brine (80 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~20% Ethyl acetate/Petroleum ether gradient @40 mL/min) to give ethyl 5-(benzyloxycarbonylamino)-3-(trifluoromethyl)-4,5,6,7-tetrahydrobenzothiophene-2-carboxylate (3) (1.6 g, 3.74 mmol, 70.42% yield) as a colorless oil. ¹H NMR (400 MHz, CHLOROFORM-d) δ=7.45-7.29 (m, 5H), 5.12 (s, 2H), 4.87 (br d, J=6.5 Hz, 1H), 4.45-4.28 (m, 2H), 4.17-3.93 (m, 1H), 3.15 (br dd, J=3.6, 16.4 Hz, 1H), 2.90 (br t, J=6.2 Hz, 2H), 2.60 (br dd, J=7.8, 16.4 Hz, 1H), 2.20-2.05 (m, 1H), 1.95-1.81 (m, 1H), 1.36 (t, J=7.1 Hz, 3H) LCMS: m/z [M+H]+=427.9.

### Procedure for preparation of 5-(benzyloxycarbonylamino)-3-(trifluoromethyl)-4,5,6,7-tetrahydrobenzothiophene-2-carboxylic acid (4)

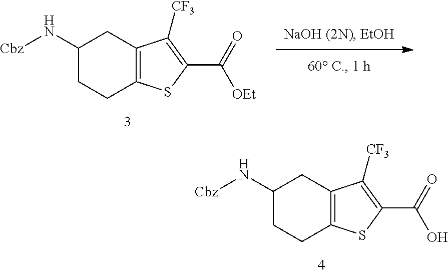 [SMILES: CCOC(=O)c1sc2c(c1C(F)(F)F)CC(NC(=O)OCc1ccccc1)CC2 ; O=C(NC1CCc2sc(C(=O)O)c(C(F)(F)F)c2C1)OCc1ccccc1]

To a solution of ethyl 5-(benzyloxycarbonylamino)-3-(trifluoromethyl)-4,5,6,7-tetrahydrobenzothiophene-2-carboxylate (3) (1.5 g, 3.51 mmol, 1 eq) in EtOH (15 mL) was added NaOH (2 M in H₂O, 5.26 mL, 3 eq). The mixture was stirred at 60° C. for 1 hr. The reaction mixture was concentrated under reduced pressure to remove ethanol and then diluted with H₂O (40 mL). Added HCl (aq. 1 M) to adjust pH=6, the solid formed, filtered and dried under reduced pressure to give 5-(benzyloxycarbonylamino)-3-(trifluoromethyl)-4,5,6,7-tetrahydrobenzothiophene-2-carboxylic acid (4) (1.4 g, crude) as an off-white solid. LCMS: m/z [M+H]⁺=399.9

### Procedure for preparation of benzyl N-[3-(trifluoromethyl)-4,5,6,7-tetrahydrobenzothiophen-5-yl]carbamate (5)

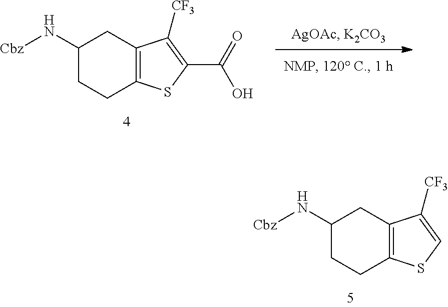 [SMILES: O=C(NC1CCc2sc(C(=O)O)c(C(F)(F)F)c2C1)OCc1ccccc1 ; O=C(NC1CCc2scc(C(F)(F)F)c2C1)OCc1ccccc1]

To a solution of 5-(benzyloxycarbonylamino)-3-(trifluoromethyl)-4,5,6,7-tetrahydrobenzothiophene-2-carboxylic acid (4) (1.4 g, 3.51 mmol, 1 eq) in NMP (15 mL) was added AgOAc (58.51 mg, 350.54 μmol, 0.1 eq) and K₂CO₃ (1.45 g, 10.52 mmol, 3 eq). The mixture was stirred at 120° C. for 1 hr. The reaction mixture was filtered, the filtrate was diluted with H₂O (60 mL), adjusted pH to 6 with HCl (aq. 1 M) and mixture extracted with EtOAc (30 mL*2). The combined organic layers were washed with brine (30 mL), dried over anhydrous Na₂SO₄, filtered and concentrated under reduced pressure. The residue was purified by flash silica gel chromatography (ISCO®; 12 g SepaFlash® Silica Flash Column, eluent 0~15% Ethyl acetate/Petroleum ether gradient @30 mL/min) to give benzyl N-[3-(trifluoromethyl)-4,5,6,7-tetrahydrobenzothiophen-5-yl]carbamate (5) (1.1 g, 3.10 mmol, 88.30% yield) as a white solid. ¹H NMR (400 MHz, CHLOROFORM-d) δ=7.52 (s, 1H), 7.42-7.31 (m, 5H), 5.12 (s, 2H), 4.88 (br d, J=6.1 Hz, 1H), 4.15-4.08 (m, 1H), 3.09 (br dd, J=4.9, 16.4 Hz, 1H), 2.94-2.82 (m, 2H), 2.57 (br dd, J=7.4, 16.4 Hz, 1H), 2.17-2.08 (m, 1H), 1.98-1.83 (m, 1H). LCMS: m/z [M+H]⁺=355.8

### Procedure for preparation of 3-(trifluoromethyl)-4,5,6,7-tetrahydrobenzothiophen-5-amine hydrochloride (186)

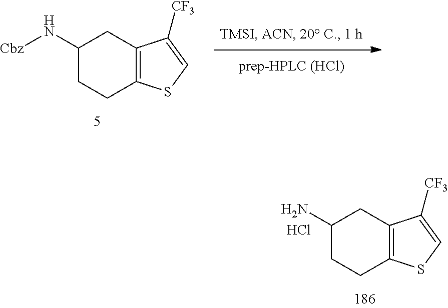 [SMILES: O=C(NC1CCc2scc(C(F)(F)F)c2C1)OCc1ccccc1 ; Cl ; NC1CCc2scc(C(F)(F)F)c2C1]

To a solution of benzyl N-[3-(trifluoromethyl)-4,5,6,7-tetrahydrobenzothiophen-5-yl]carbamate (5) (80 mg, 225.11 μmol, 1 eq) in CH₃CN (1 mL) was added TMSI (180.18 mg, 900.46 μmol, 122.57 μL, 4 eq). The mixture was stirred at 25° C. for 1 hr. The mixture was concentrated and aq. sat. NaHCO₃ (0.5 mL) was added. DMF (2 mL) was added insoluble substances were filtered off. The DMF solution was purified by prep-HPLC (column: Phenomenex luna C18 150*25 mm*10 μm; mobile phase: [water(HCl)-ACN]; gradient: 1%-30% B over 10 min) to afford 3-(trifluoromethyl)-4,5,6,7-tetrahydrobenzothiophen-5-amine hydrochloride (186) (17.78 mg, 68.30 μmol, 30.34% yield) as a white solid. ¹H NMR (400 MHz, METHANOL-d4) δ=7.85 (s, 1H), 3.73-3.58 (m, 1H), 3.20 (br dd, J=5.1, 16.0 Hz, 1H), 3.08-2.91 (m, 2H), 2.72 (br dd, J=8.9, 15.7 Hz, 1H), 2.33-2.20 (m, 1H), 2.04-1.89 (m, 1H). LCMS: m/z [M+H]⁺=222.1

### Synthetic scheme for preparation of 3-chloro-4,5,6,7-tetrahydrobenzothiophen-5-amine hydrochloride (187)

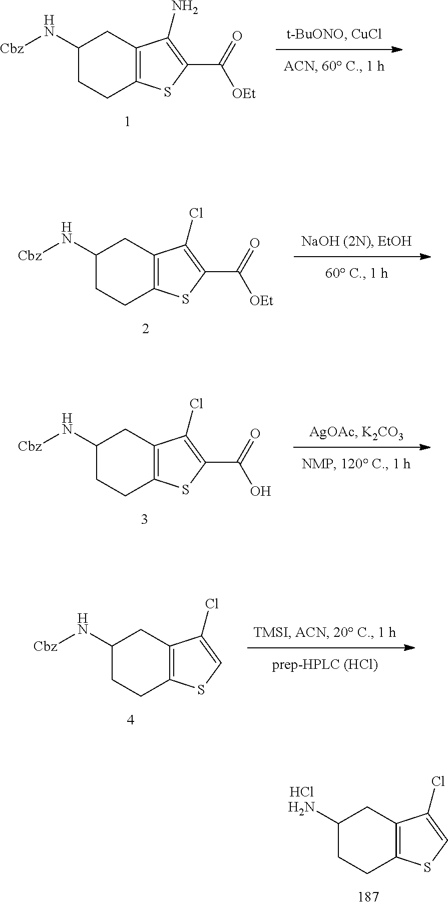 [SMILES: CCOC(=O)c1sc2c(c1N)CC(NC(=O)OCc1ccccc1)CC2 ; CCOC(=O)c1sc2c(c1Cl)CC(NC(=O)OCc1ccccc1)CC2 ; O=C(NC1CCc2sc(C(=O)O)c(Cl)c2C1)OCc1ccccc1 ; O=C(NC1CCc2scc(Cl)c2C1)OCc1ccccc1 ; Cl ; NC1CCc2scc(Cl)c2C1]

### Procedure for preparation of ethyl 5-(benzyloxycarbonylamino)-3-chloro-4,5,6,7-tetrahydrobenzothiophene-2-carboxylate (2)

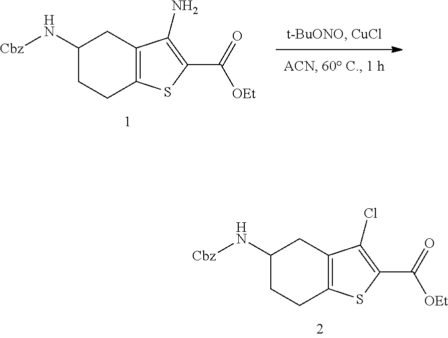 [SMILES: CCOC(=O)c1sc2c(c1N)CC(NC(=O)OCc1ccccc1)CC2 ; CCOC(=O)c1sc2c(c1Cl)CC(NC(=O)OCc1ccccc1)CC2]

To a solution of ethyl 3-amino-5-(benzyloxycarbonylamino)-4,5,6,7-tetrahydrobenzothiophene-2-carboxylate (1) (6 g, 16.02 mmol, 1 eq) in ACN (100 mL) was added CuCl (4.8 g, 48.1 mmol, 3 eq) and t-BuONO (2.5 g, 24 mmol, 2.9 mL, 1.5 eq). The mixture was stirred at 60° C. for 1 hr. The reaction mixture was concentrated under reduced pressure to give a residue. The residue was purified by flash silica gel chromatography (ISCO®; 40 g SepaFlash® Silica Flash Column, eluent 0~25% Ethyl acetate/Petroleum ether gradient @40 mL/min) to give ethyl 5-(benzyloxycarbonylamino)-3-chloro-4,5,6,7-tetrahydrobenzothiophene-2-carboxylate (187) (3.1 g, 4.7 mmol, 30.80% yield) as a yellow oil. ¹H NMR (400 MHz, CHLOROFORM-d) δ=7.39-7.32 (m, 5H), 5.12 (br s, 2H), 4.84 (br d, J=5.9 Hz, 1H), 4.38-4.30 (m, 2H), 4.12-4.05 (m, 1H), 3.03-2.81 (m, 3H), 2.42 (br dd, J=7.6, 16.4 Hz, 1H), 2.11 (br dd, J=4.8, 9.4 Hz, 1H), 1.96-1.83 (m, 1H), 1.40-1.33 (t, 3H). LCMS: m/z [M+H]⁺=393.9

### Procedure for preparation of 5-(benzyloxycarbonylamino)-3-chloro-4,5,6,7-tetrahydrobenzothiophene-2-carboxylic acid (3)

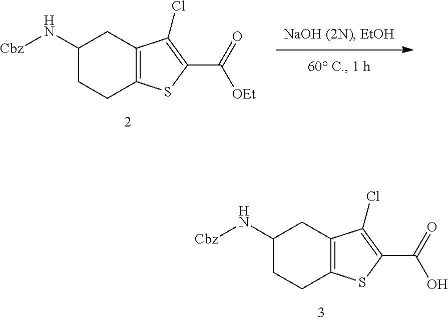 [SMILES: CCOC(=O)c1sc2c(c1Cl)CC(NC(=O)OCc1ccccc1)CC2 ; O=C(NC1CCc2sc(C(=O)O)c(Cl)c2C1)OCc1ccccc1]

To a solution of ethyl 5-(benzyloxycarbonylamino)-3-chloro-4,5,6,7-tetrahydrobenzothiophene-2-carboxylate (2) (3.1 g, 4.8 mmol, 1 eq, 61% purity) in EtOH (30 ml) was added NaOH (2 M in H₂O, 7.87 mL). The mixture was stirred at 60° C. for 1 hr. The reaction mixture was concentrated under reduced pressure to remove EtOH. The residue was diluted with H₂O (20 mL). Added HCl (aq. 1 M) to adjust pH to 5. The solid that separated out, was filtered and dried under reduced pressure to give 5-(benzyloxycarbonylamino)-3-chloro-4,5,6,7-tetrahydrobenzothiophene-2-carboxylic acid (3) (2.9 g, crude) as a white solid. LCMS: m/z [M-CO₂]⁺=321.8

### Procedure for preparation of benzyl N-(3-chloro-4,5,6,7-tetrahydrobenzothiophen-5-yl)carbamate (4)

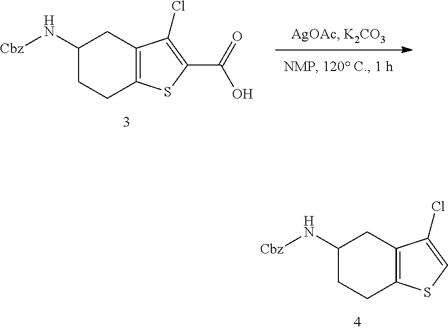 [SMILES: O=C(NC1CCc2sc(C(=O)O)c(Cl)c2C1)OCc1ccccc1 ; O=C(NC1CCc2scc(Cl)c2C1)OCc1ccccc1]

To a solution of 5-(benzyloxycarbonylamino)-3-chloro-4,5,6,7-tetrahydrobenzothiophene-2-carboxylic acid (3) (2.9 g, crude, 1 eq) in NMP (30 mL) was added AgOAc (132 mg, 793 μmol, 0.1 eq) and K₂CO₃ (3.3 g, 23.78 mmol, 3 eq). The mixture was stirred at 120° C. for 1 hr under N₂. The reaction mixture was filtered, the filtrate was diluted with H₂O (150 mL), then adjusted pH to 6 with HCl (aq. 1 M) and extracted with EtOAc (40 mL*2). The combined organic layers were washed with brine (30 mL), dried over anhydrous Na₂SO₄, filtered and concentrated under reduced pressure to give a residue. The residue was purified by flash silica gel chromatography (ISCO®; 24 g SepaFlash® Silica Flash Column, eluent 0~15% Ethyl acetate/Petroleum ether gradient @30 mL/min) to give benzyl N-(3-chloro-4,5,6,7-tetrahydrobenzothiophen-5-yl)carbamate (4) (1.2 g, 3.73 mmol, 47.04% yield) as a white solid. ¹H NMR (400 MHz, CHLOROFORM-d) δ=7.46-7.31 (m, 5H), 6.95 (s, 1H), 5.12 (s, 2H), 4.94-4.81 (m, 1H), 4.21-4.06 (m, 1H), 3.01-2.80 (m, 3H), 2.41 (br dd, J=7.1, 16.3 Hz, 1H), 2.16-2.03 (m, 1H), 1.97-1.81 (m, 1H). LCMS: m/z [M+H]⁺=321.8

### Procedure for preparation of 3-chloro-4,5,6,7-tetrahydrobenzothiophen-5-amine hydrochloride (187)

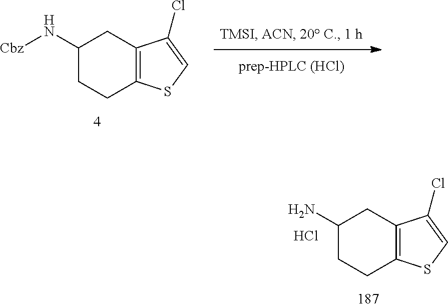 [SMILES: O=C(NC1CCc2scc(Cl)c2C1)OCc1ccccc1 ; Cl ; NC1CCc2scc(Cl)c2C1]

To a solution of benzyl N-(3-chloro-4,5,6,7-tetrahydrobenzothiophen-5-yl)carbamate (4) (80 mg, 248.59 μmol, 1 eq) in ACN (1 mL) was added TMSI (198.96 mg, 994.34 μmol, 135.35 μL, 4 eq). The mixture was stirred at 25° C. for 1 hr. The mixture was concentrated and aq. sat. NaHCO₃ (0.5 mL) was added. DMF (2 mL) was added insoluble substances were filtered off. The DMF solution was purified by prep-HPLC (column: Phenomenex luna C18 150*25 mm*10 μm; mobile phase: [water(HCl)-ACN]; gradient:1%-25% B over 10 min) to afford 3-chloro-4,5,6,7-tetrahydrobenzothiophen-5-amine hydrochloride (187) (27.37 mg, 144.37 μmol, 58.08% yield as a white solid. ¹H NMR (400 MHz, METHANOL-d4) δ=7.19 (s, 1H), 3.62 (dddd, J=2.9, 5.6, 8.4, 11.0 Hz, 1H), 3.11-2.90 (m, 3H), 2.54 (br dd, J=9.1, 16.0 Hz, 1H), 2.31-2.19 (m, 1H), 2.03-1.88 (m, 1H). LCMS: m/z [M+H]⁺=188.1 Synthetic scheme for preparation of 2-methyl-6,7-dihydro-5H-thieno[3,2-b]pyran-6-amine hydrochloride (188)

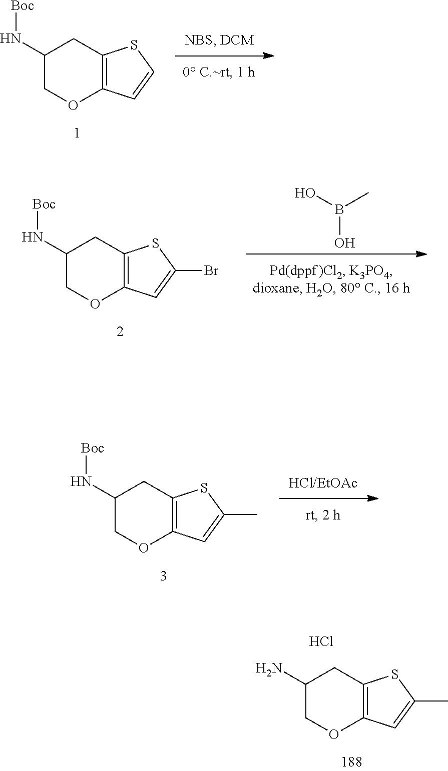

### Procedure for preparation of tert-butyl N-(2-bromo-6,7-dihydro-5H-thieno[3,2-b]pyran-6-yl)carbamate (2)

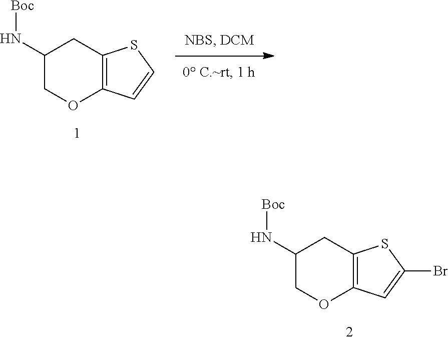 [SMILES: CC(C)(C)OC(=O)NC1COc2ccsc2C1 ; CC(C)(C)OC(=O)NC1COc2cc(Br)sc2C1]

To a solution of tert-butyl N-(6,7-dihydro-5H-thieno[3,2-b]pyran-6-yl)carbamate (1) (800 mg, 3.13 mmol, 1 eq) in DCM (10 mL) was added NBS (669.17 mg, 3.76 mmol, 1.2 eq) at 0° C. The mixture was allowed to warm to −25° C. and stirred for 1 hr. The reaction mixture was concentrated under reduced pressure to remove DCM. The residue was purified by flash silica gel chromatography (ISCO®; 12 g SepaFlash® Silica Flash Column, eluent 0~20% Ethyl acetate/Petroleum ether gradient @40 mL/min) to afford tert-butyl N-(2-bromo-6,7-dihydro-5H-thieno[3,2-b]pyran-6-yl)carbamate (2) (1 g, 2.99 mmol, 95.49% yield) as yellow oil. ¹H NMR (400 MHz, CHLOROFORM-d) δ=6.63 (s, 1H), 5.02-4.89 (m, 1H), 4.29-4.17 (m, 1H), 4.12-3.98 (m, 2H), 2.97 (dd, J=5.4, 16.4 Hz, 1H), 2.62 (br d, J=16.4 Hz, 1H), 1.45 (s, 9H)

### Procedure for preparation of tert-butyl N-(2-methyl-6,7-dihydro-5H-thieno[3,2-b]pyran-6-yl)carbamate (3)

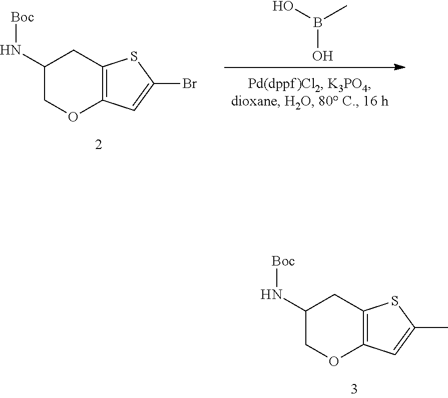

A mixture of tert-butyl N-(2-bromo-6,7-dihydro-5H-thieno[3,2-b]pyran-6-yl)carbamate (2) (1 g, 2.99 mmol, 1 eq) and methylboronic acid (895.49 mg, 14.96 mmol, 5 eq) was added Pd(dppf)Cl₂ (109.46 mg, 149.60 μmol, 0.05 eq) and K₃PO₄ (1.91 g, 8.98 mmol, 3 eq) in dioxane (10 mL) and H₂O (2 mL). Mixture was degassed and purged with N₂ for 3 times, and then the mixture was stirred at 80° C. for 16 hrs under N₂ atmosphere. The reaction mixture was poured into H₂O (30 mL) and extracted with EtOAc (20 mL*3). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0-20% Ethyl acetate/Petroleum ether gradient @40 mL/min). and then by prep-HPLC (column: Waters Xbridge 150*25 mm*5 μm; mobile phase: [water(NH₄HCO₃)-ACN]; gradient:48%-78% B over 9 min) to afford tert-butyl N-(2-methyl-6,7-dihydro-5H-thieno[3,2-b]pyran-6-yl)carbamate (3) (170 mg, 631.13 μmol, 21.09% yield) as a white solid. ¹H NMR (400 MHz, CHLOROFORM-d) δ=6.32 (s, 1H), 5.09-4.92 (m, 1H), 4.29-3.96 (m, 3H), 3.13-2.95 (m, 1H), 2.62 (br d, J=15.5 Hz, 1H), 2.39 (s, 3H), 1.45 (s, 9H).

### Procedure for preparation of 2-methyl-6,7-dihydro-5H-thieno[3,2-b]pyran-6-amine hydrochloride (188)

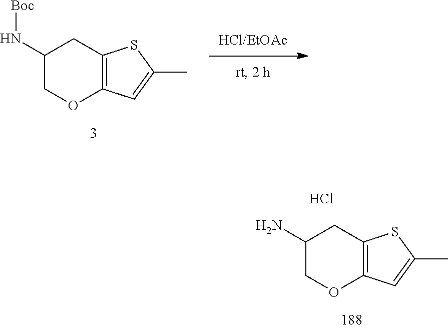 [SMILES: Cc1cc2c(s1)CC(NC(=O)OC(C)(C)C)CO2 ; Cl ; Cc1cc2c(s1)CC(N)CO2]

To a solution of tert-butyl N-(2-methyl-6,7-dihydro-5H-thieno[3,2-b]pyran-6-yl)carbamate (3) (70 mg, 259.88 μmol, 1 eq) in EtOAc (1 mL) was added HCl/EtOAc (4 M, 1.17 mL, 17.96 eq). The mixture was stirred at 25° C. for 2 hr. The mixture was then concentrated and the residue was triturated with EtOAc (2 mL) at 25° C. for 10 min. Followed filtration, the filter cake was dried and collected to afford 2-methyl-6,7-dihydro-5H-thieno[3,2-b]pyran-6-amine hydrochloride (188) (50.91 mg, 242.54 μmol, 93.33% yield) as a white solid. ¹H NMR (400 MHz, METHANOL-d4) δ=6.43-6.31 (m, 1H), 4.34-4.08 (m, 2H), 3.93-3.80 (m, 1H), 3.26-3.11 (m, 1H), 2.83-2.68 (m, 1H), 2.37 (s, 3H). LCMS: m/z [M+H]⁺=170.1

### Synthetic scheme for preparation of 2-chloro-N,N-dimethyl-4,5,6,7-tetrahydrobenzothiophen-5-amine hydrochloride (189)

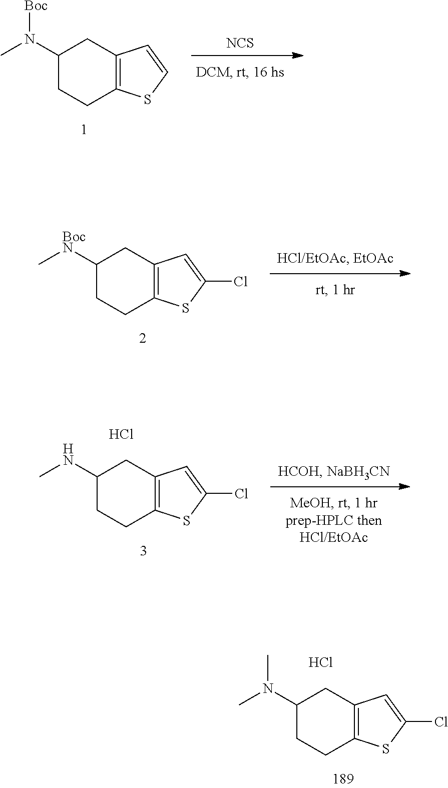

### Procedure for preparation of tert-butyl N-(2-chloro-4,5,6,7-tetrahydrobenzothiophen-5-yl)-N-methyl-carbamate (2)

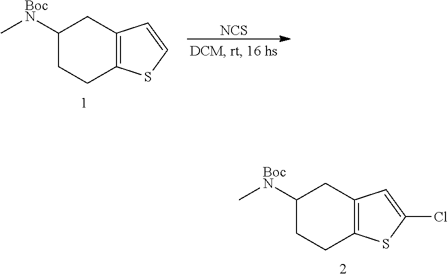 [SMILES: CN(C(=O)OC(C)(C)C)C1CCc2sccc2C1 ; "NCS" ; CN(C(=O)OC(C)(C)C)C1CCc2sc(Cl)cc2C1]

To a solution of tert-butyl N-methyl-N-(4,5,6,7-tetrahydrobenzothiophen-5-yl)carbamate (1) (1.8 g, 6.73 mmol, 1 eq) in DCM (20 mL) was added NCS (898.90 mg, 6.73 mmol, 1 eq). The mixture was stirred at 25° C. for 16 hr. The reaction mixture was concentrated under reduced pressure and then diluted with H₂O (30 mL). The mixture was extracted with EtOAc (10 mL*3). The combined organic layers were washed with brine (20 mL), dried over anhydrous sodium sulfate, filtered and concentrated under reduced pressure to give a residue. The residue was purified by flash silica gel chromatography (ISCO®; 10 g SepaFlash® Silica Flash Column, eluent 0~14% Ethyl acetate/Petroleum ether gradient @24 mL/min). Compound tert-butyl N-(2-chloro-4,5,6,7-tetrahydrobenzothiophen-5-yl)-N-methyl-carbamate (2) (1.6 g, 5.30 mmol, 78.74% yield) was obtained as a yellow oil. ¹H NMR (400 MHz, CHLOROFORM-d) δ=6.55 (s, 1H), 4.62-4.18 (m, 1H), 2.99-2.72 (m, 5H), 2.70-2.54 (m, 2H), 2.03-1.80 (m, 2H), 1.48 (s, 9H).

### Procedure for preparation of 2-chloro-N-methyl-4,5,6,7-tetrahydrobenzothiophen-5-amine (3)

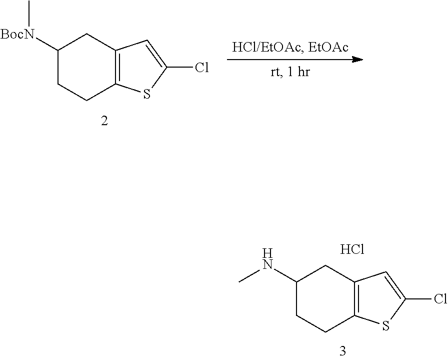 [SMILES: CN(C(=O)OC(C)(C)C)C1CCc2sc(Cl)cc2C1 ; Cl ; CNC1CCc2sc(Cl)cc2C1]

To a solution of tert-butyl N-(2-chloro-4,5,6,7-tetrahydrobenzothiophen-5-yl)-N-methyl-carbamate (2) (200 mg, 694 μmol, 1 eq) in EtOAc (2 mL) was added HCl/EtOAc (4 M, 2 mL). The mixture was stirred at 25° C. for 1 hr. The reaction mixture was then concentrated under vacuum to give the crude product. Compound 2-chloro-N-methyl-4,5,6,7-tetrahydrobenzothiophen-5-amine hydrochloride (3) (0.18 g, crude) was obtained as a white solid.

### Procedure for preparation of 2-chloro-N, N-dimethyl-4,5,6,7-tetrahydrobenzothiophen-5-amine hydrochloride (189)

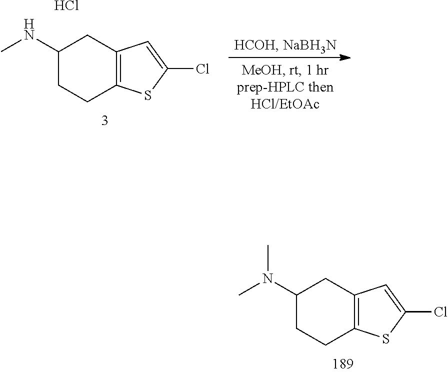

To a solution of 2-chloro-N-methyl-4,5,6,7-tetrahydrobenzothiophen-5-amine hydrochloride (3) (0.15 g, 743.62 μmol, 1 eq) in MeOH (2 mL) was added formaldehyde (44.66 mg, 1.49 mmol, 40.97 μL, 2 eq) and NaBH₃CN (186.92 mg, 2.97 mmol, 4 eq). The mixture was stirred at 25° C. for 1 hr. The reaction mixture was concentrated under reduced pressure to remove solvent. The residue was treated with H₂O (10 mL) and extracted with EtOAc (10 mL*3). The combined organic layers were washed with brine (10 mL), dried over anhydrous sodium sulfate, filtered and concentrated under reduced pressure to give a residue. The residue was purified by pre-HPLC (column: Waters Xbridge 150*25 mm*5 μm; mobile phase: [water (ammonia hydroxide v/v)-ACN]; gradient: 30%-60% B over min). Fractions containing desired product were concentrated. The residue was diluted with EtOAc (1 mL), and HCl/EtOAc (4M, 1 mL) was added. The mixture formed was concentrated under reduced pressure to give the product. Compound 2-chloro-N,N-dimethyl-4,5,6,7-tetrahydrobenzothiophen-5-amine hydrochloride (189) (59.39 mg, 274.54 μmol, 36.92% yield) was obtained as a white solid. ¹H NMR (400 MHz, METHANOL-d₄) δ =6.72 (s, 1H), 3.72-3.59 (m, 1H), 3.15-2.92 (m, 8H), 2.91-2.75 (m, 2H), 2.39-2.31 (m, 1H), 1.99 (dq, J=5.7, 11.8 Hz, 1H), LC-MS: m/z [M+H]⁺=216.1.

### Synthetic scheme for preparation of N, 5-dimethyl-3-(trifluoromethyl)-6,7-dihydro-4H-benzothiophen-5-amine (190)

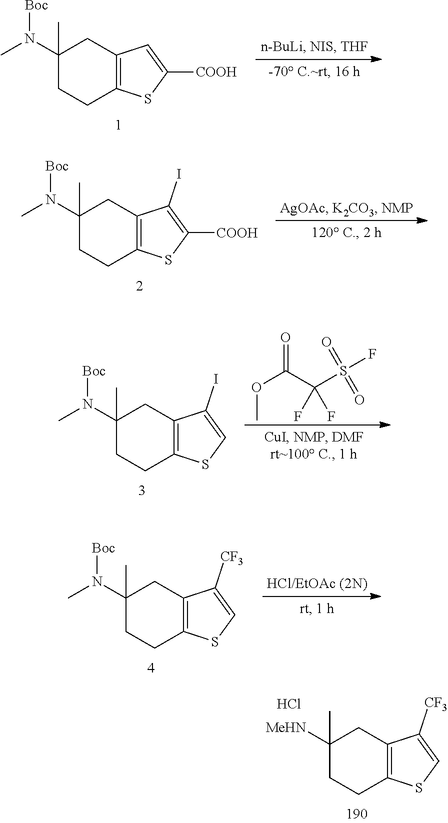

### Procedure for preparation of 5-[tert-butoxycarbonyl(methyl)amino]-3-iodo-5-methyl-6,7-dihydro-4H-benzothiophene-2-carboxylic acid (2)

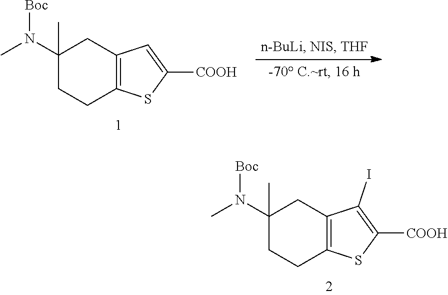 [SMILES: CN(C(=O)OC(C)(C)C)C1(C)CCc2sc(C(=O)O)cc2C1 ; CN(C(=O)OC(C)(C)C)C1(C)CCc2sc(C(=O)O)c(I)c2C1]

To the solution of 5-[tert-butoxycarbonyl(methyl)amino]-5-methyl-6,7-dihydro-4H-benzothiophene-2-carboxylic acid (1) (1 g, 3.07 mmol, 1.00 eq) in THF (15 mL) was added n-BuLi (2.5 M, 2.95 mL, 2.40 eq) at −70° C. under atmosphere. After stirring for 1 h, a solution of NIS (1.26 g, 5.60 mmol, 1.82 eq) in THF (5 mL) was added drop-wise at −70° C. and the resulting mixture was stirred for 1 h at −70° C. Then the mixture was warmed to room temperature slowly and stirred for 16 h. The reaction mixture was poured into sat. NH₄Cl (15 mL), diluted with water (15 mL), adjusted pH to 3-4 with citric acid and extracted with EtOAc (15 mL*3). The combined organic layers were washed with brine (15 mL), dried over anhydrous sodium sulfate, filtered and concentrated under vacuum to give 5-[tert-butoxycarbonyl(methyl)amino]-3-iodo-5-methyl-6,7-dihydro-4H-benzothiophene-2-carboxylic acid (2) (1.5 g, crude) which was used into next step without further purification. ¹H NMR (400 MHz, CDCl₃-d) δ 3.20-3.02 (m, 2H), 2.86-2.80 (m, 2H), 2.68 (s, 3H), 2.50 (d, J=17.2 Hz, 1H), 1.81-1.71 (m, 1H), 1.49-1.46 (m, 12H).

### Procedure for preparation of tert-butyl N-(3-iodo-5-methyl-6,7-dihydro-4H-benzothiophen-5-yl)-N-methyl-carbamate (3)

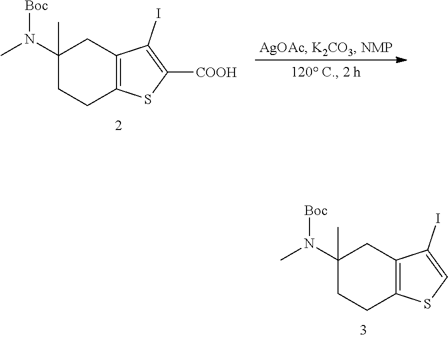 [SMILES: CN(C(=O)OC(C)(C)C)C1(C)CCc2sc(C(=O)O)c(I)c2C1 ; CN(C(=O)OC(C)(C)C)C1(C)CCc2scc(I)c2C1]

To the mixture of 5-[tert-butoxycarbonyl(methyl)amino]-3-iodo-5-methyl-6,7-dihydro-4H-benzothiophene-2-carboxylic acid (2) (500 mg, 1.11 mmol, 1.00 eq) in NMP (5 mL) was added AgOAc (37 mg, 221 μmol, 0.20 eq) and K₂CO₃ (459 mg, 3.32 mmol, 3.00 eq). The resulting mixture was stirred at 120° C. for 2 h. The reaction mixture was diluted with water (20 mL) and extracted with EtOAc (10 mL*3). The combined organic phases were washed with brine (15 mL), dried over anhydrous sodium sulfate, filtered and concentrated under vacuum to give a residue. The residue was purified by flash silica gel chromatography (ISCO®; 4 g SepaFlash® Silica Flash Column, eluent 0~15% Ethyl acetate/Petroleum ether gradient @20 mL/min) to give tert-butyl N-(3-iodo-5-methyl-6,7-dihydro-4H-benzothiophen-5-yl)-N-methyl-carbamate (3) (350 mg, 859 μmol, 77.6% yield) as a yellow solid. ¹H NMR (400 MHz, CDCl₃-d) δ 7.18 (s, 1H), 2.98-2.83 (m, 2H), 2.73-2.68 (m, 2H), 2.61 (s, 3H), 2.40 (d, J=16.8 Hz, 1H), 1.66 (td, J=7.6, 12.8 Hz, 1H), 1.41-1.39 (m, 9H), 1.37 (s, 3H). LCMS: m/z [M+H-Boc]⁺=307.9.

### Procedure for preparation of tert-butyl N-methyl-N-[5-methyl-3-(trifluoromethyl)-6,7-dihydro-4H-benzothiophen-5-yl]carbamate (4)

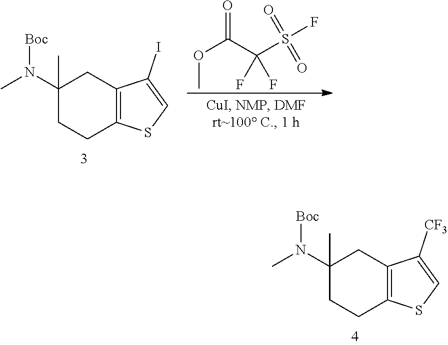

To a solution of tert-butyl N-(3-iodo-5-methyl-6,7-dihydro-4H-benzothiophen-5-yl)-N-methyl-carbamate (3) (300 mg, 737 μmol, 1.00 eq) in DMF (6 mL) was added CuI (154 mg, 810 μmol, 1.10 eq) and NMP (365 mg, 3.68 mmol, 5.00 eq) at room temperature. After addition, the mixture was stirred at this temperature for 15 min, and then methyl 2,2-difluoro-2-fluorosulfonyl-acetate (707 mg, 3.68 mmol, 5.00 eq) was added dropwise at room temperature. The resulting mixture was stirred at 100° C. for 1 hr. The reaction mixture was diluted with water (30 mL) and extracted with EtOAc (15 mL*3). The combined organic layers were washed with brine (20 mL), dried over anhydrous sodium sulfate, filtered and concentrated under vacuum to give a residue. The residue was purified by prep-HPLC (column: Phenomenex luna C18 250*50 mm*15 μm; mobile phase: [water(FA)-ACN]; gradient: 70%-100% B over 7 min) to give tert-butyl N-methyl-N-[5-methyl-3-(trifluoromethyl)-6,7-dihydro-4H-benzothiophen-5-yl]carbamate (4) (200 mg, 572 μmol, 77.7% yield) as a light yellow solid. ¹H NMR (400 MHz, CDCl₃-d) δ 7.51 (s, 1H), 3.21 (d, J=17.2 Hz, 1H), 3.12-2.98 (m, 1H), 2.83-2.74 (m, 2H), 2.72 (s, 3H), 2.63 (d, J=17.2 Hz, 1H), 1.75 (td, J=8.0, 13.2 Hz, 1H), 1.47 (s, 9H), 1.45 (s, 3H). LCMS: m/z [M+H-Boc]⁺=250.0.

### Procedure for preparation of N, 5-dimethyl-3-(trifluoromethyl)-6,7-dihydro-4H-benzothiophen-5-amine hydrochloride

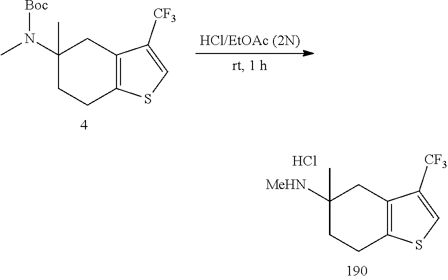 [SMILES: CN(C(=O)OC(C)(C)C)C1(C)CCc2scc(C(F)(F)F)c2C1 ; Cl ; CNC1(C)CCc2scc(C(F)(F)F)c2C1]

To the solution of tert-butyl N-methyl-N-[5-methyl-3-(trifluoromethyl)-6,7-dihydro-4H-benzothiophen-5-yl]carbamate (4) (200 mg, 572 μmol, 1.00 eq) in EtOAc (2 mL) was added HCl/EtOAc (4 M, 2 mL). The mixture was stirred at room temperature for 1 h. The reaction mixture was filtered. The filter cake was washed with EtOAc (10 mL) and dried under vacuum to give N, 5-dimethyl-3-(trifluoromethyl)-6,7-dihydro-4H-benzothiophen-5-amine hydrochloride (190) (105.49 mg, 406.69 μmol, 71.1% yield) as an off-white solid. ¹H NMR (400 MHz, MeOD-d₄) δ 7.89 (s, 1H), 3.04-2.94 (m, 4H), 2.72 (s, 3H), 2.14 (t, J=6.4 Hz, 2H), 1.43 (s, 3H). LCMS: m/z [M+H]⁺=250.0.

### Synthetic scheme for preparation of 3-fluoro-4,5,6,7-tetrahydrobenzothiophen-5-amine hydrochloride enantiomer 1 (191) and enantiomer 2 (192)

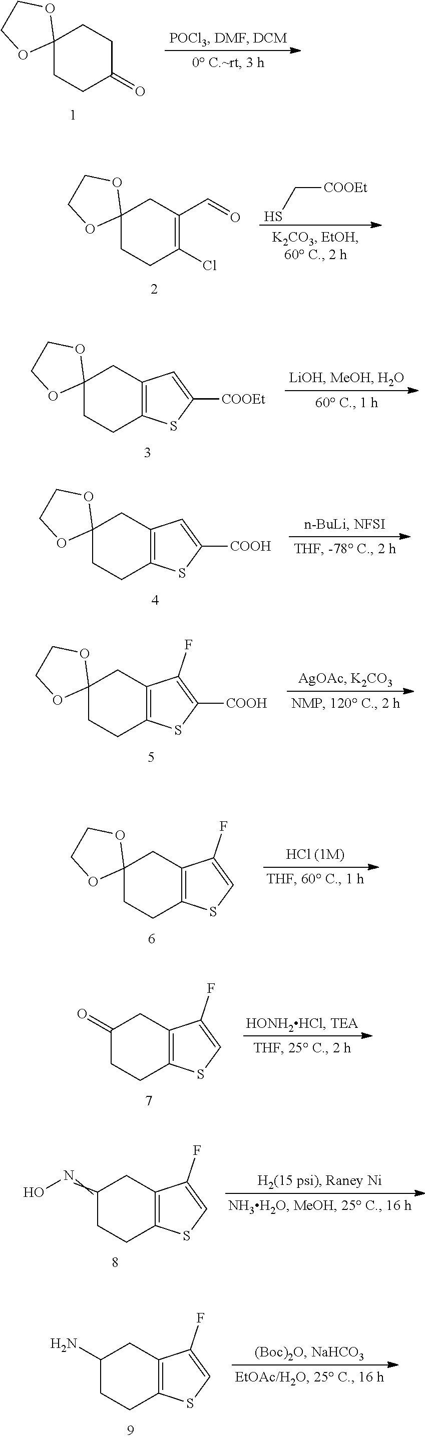

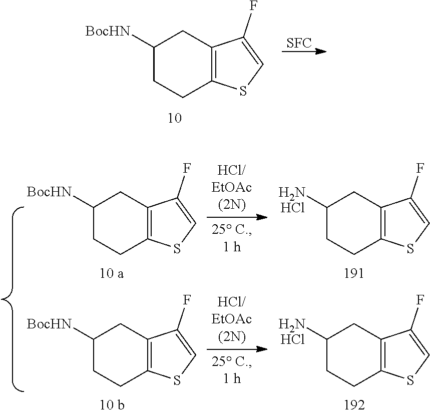

### Procedure for preparation of 8-chloro-1,4-dioxaspiro[4.5]dec-7-ene-7-carbaldehyde (2)

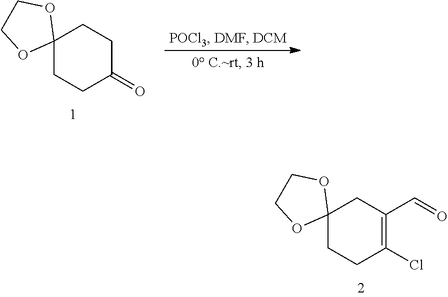 [SMILES: O=C1CCC2(CC1)OCCO2 ; O=CC1=C(Cl)CCC2(C1)OCCO2]

To a solution of DMF (70.20 g, 960.44 mmol, 73.90 mL, 6 eq) in DCM (300 ml) was added POCl₃ (122.72 g, 800.37 mmol, 74.60 mL, 5 eq) at 0° C. The mixture was, stirred at 20° C. for 1 h, then it was added 1,4-dioxaspiro[4.5]decan-8-one (1) (25 g, 160.07 mmol, 1 eq) in DCM (50 mL) at 0° C. The mixture was stirred at 25° C. for 2 h. The reaction mixture was diluted with aq. sat. NaHCO₃ (1000 mL) and extracted with DCM (200 mL*2). The combined organic layers were washed with aq. sat. NaCl (100 mL), dried over anhydrous Na₂SO₄, filtered and concentrated under reduced pressure. The residue was purified by flash silica gel chromatography (ISCO; 80 g SepaFlash Silica Flash Column, eluent 0~11% Ethyl acetate/Petroleum ether gradient @40 mL/min). Compound 8-chloro-1,4-dioxaspiro[4.5]dec-7-ene-7-carbaldehyde (2) (26 g, 128.31 mmol, 80.16% yield) was obtained as a yellow oil. ¹H NMR (400 MHz, CHLOROFORM-d) δ=10.21-10.05 (m, 1H), 3.99 (br s, 4H), 2.81 (br d, J=5.1 Hz, 2H), 2.49 (br s, 2H), 1.95-1.86 (m, 2H)

### Procedure for preparation of ethyl spiro[1,3-dioxolane-2,5′-6,7-dihydro-4H-benzothiophene]-2′-carboxylate (3)

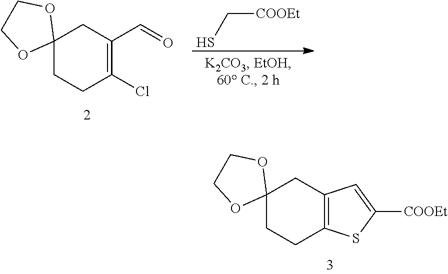

To a solution of 8-chloro-1,4-dioxaspiro[4.5]dec-7-ene-7-carbaldehyde (2) (25 g, 123.37 mmol, 1 eq) and K₂CO₃ (17.05 g, 123.37 mmol, 1 eq) in EtOH (200 mL) was added ethyl 2-sulfanylacetate (20.61 g, 171.51 mmol, 18.80 mL, 1.39 eq). The mixture was stirred at 60° C. for 2 hr. The reaction mixture was diluted with sat. NaHCO₃ 1000 mL and extracted with DCM (200 mL*2). The combined organic layers were washed with sat. NaCl (100 mL), dried over anhydrous Na₂SO₄, filtered and concentrated under reduced pressure to give a residue. The residue was purified by flash silica gel chromatography (ISCO; 80 g SepaFlash Silica Flash Column, eluent 0~11% Ethyl acetate/Petroleum ether gradient @40 mL/min). Compound ethyl spiro[1,3-dioxolane-2,5′-6,7-dihydro-4H-benzothiophene]-2′-carboxylate (3) (30 g, 111.80 mmol, 90.62% yield) was obtained as a colorless oil. LCMS: m/z [M+H]⁺=269.2. ¹H NMR (400 MHz, CHLOROFORM-d) δ=7.44 (s, 1H), 4.31 (q, J=7.1 Hz, 2H), 4.05-4.01 (m, 4H), 2.99 (t, J=6.6 Hz, 2H), 2.85 (s, 2H), 2.00 (t, J=6.6 Hz, 2H), 1.35 (t, J=7.1 Hz, 3H)

### Procedure for preparation of spiro[1,3-dioxolane-2,5′-6,7-dihydro-4H-benzothiophene]-2′-carboxylic acid (4)

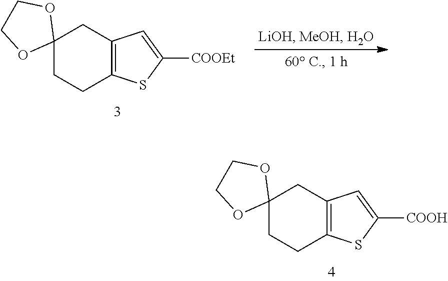 [SMILES: CCOC(=O)c1cc2c(s1)CCC1(C2)OCCO1 ; O=C(O)c1cc2c(s1)CCC1(C2)OCCO1]

To a solution of ethyl spiro[1,3-dioxolane-2,5′-6,7-dihydro-4H-benzothiophene]-2′-carboxylate (3) (15 g, 55.90 mmol, 1 eq) in MeOH (80 mL) and H₂O (80 mL) was added LiOH·H₂O (9.38 g, 223.61 mmol, 4 eq). The mixture was stirred at 60° C. for 1 hr. The reaction mixture was concentrated under vacuum to remove MeOH, and diluted with water (50 ml). The aqueous phase pH was adjusted to ~7 with HCl (1 mol/L in water), and then then to 3~4 with citric acid and then filtered. The cake was washed with water (50 mL) and collected under vacuum to give the product. Compound spiro[1,3-dioxolane-2,5′-6,7-dihydro-4H-benzothiophene]-2′-carboxylic acid (4) (10.6 g, 44.12 mmol, 78.92% yield) was obtained as a white solid. LCMS: m/z [M+H]⁺=241.0. ¹H NMR (400 MHz, CHLOROFORM-d) δ=7.53 (s, 1H), 4.09-4.00 (m, 4H), 3.03 (t, J=6.5 Hz, 2H), 2.88 (s, 2H), 2.02 (t, J=6.6 Hz, 2H)

### Procedure for preparation of 3′-fluorospiro[1,3-dioxolane-2,5′-6,7-dihydro-4H-benzothiophene]-2′-carboxylic acid (5)

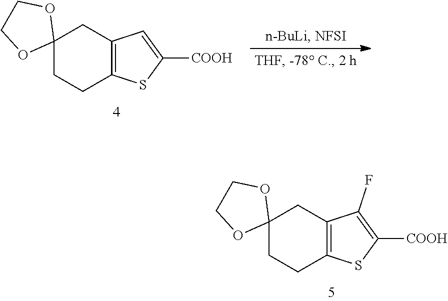 [SMILES: O=C(O)c1cc2c(s1)CCC1(C2)OCCO1 ; O=C(O)c1sc2c(c1F)CC1(CC2)OCCO1]

To a solution of spiro[1,3-dioxolane-2,5′-6,7-dihydro-4H-benzothiophene]-2′-carboxylic acid (4) (10 g, 41.62 mmol, 1 eq) in THF (100 mL) was added dropwise n-BuLi (2.5 M, 41.62 mL, 2.5 eq) at −78° C. After addition, the mixture was stirred at −78° C. for 1 hr, and then NFSI (18.37 g, 58.27 mmol, 1.4 eq) was added at −78° C. The resulting mixture was stirred at −78° C. for 1 hr. The reaction mixture was quenched with aq. sat. NH₄Cl (30 mL), and diluted with water (50 mL). The pH of mixture was about 8-9. This mixture was extracted with EtOAc (50 mL*2). Then the aqueous phase pH was adjusted to 5-6 with citric acid and extracted with EtOAc (50 mL*2). The combined organic layers were washed with aq. sat. NaCl (10 mL), dried over anhydrous Na₂SO₄, filtered and concentrated under reduced pressure. Compound 3′-fluorospiro[1,3-dioxolane-2,5′-6,7-dihydro-4H-benzothiophene]-2′-carboxylic acid (5) (11 g, crude) was obtained as a red solid. LCMS: m/z [M+H]⁺=259.0.

### Procedure for preparation of 3′-fluorospiro[1,3-dioxolane-2,5′-6,7-dihydro-4H-benzothiophene](6)

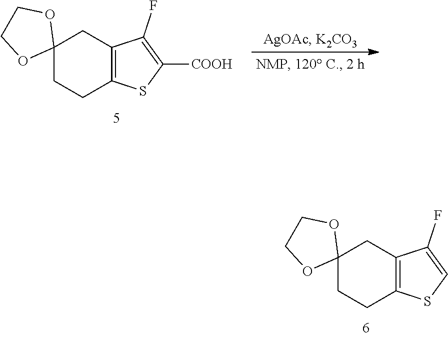 [SMILES: O=C(O)c1sc2c(c1F)CC1(CC2)OCCO1 ; Fc1csc2c1CC1(CC2)OCCO1]

To a solution of the mixture of 3′-fluorospiro[1,3-dioxolane-2,5′-6,7-dihydro-4H-benzothiophene]-2′-carboxylic acid (5) (11 g, 42.59 mmol, 1 eq) in NMP (100 mL) was added silver acetate (3.55 g, 21.30 mmol, 0.5 eq) and K₂CO₃ (17.66 g, 127.78 mmol, 3 eq). The mixture was stirred at 120° C. for 2 hr. The reaction mixture was diluted with H₂O (300 mL) and extracted with EtOAc (100 mL*2). The combined organic layers were washed with sat. NaCl (100 mL), dried over anhydrous Na₂SO₄, filtered and concentrated under reduced pressure. The residue was purified by flash silica gel chromatography (ISCO®; 4 g SepaFlash® Silica Flash Column, eluent 0~8% Ethyl acetate/Petroleum ether gradient @40 mL/min) to obtain 3′-fluorospiro[1,3-dioxolane-2,5′-6,7-dihydro-4H-benzothiophene](6) (1.2 g, 5.60 mmol, 13.15% yield) as a colorless oil. LCMS: m/z [M+H]⁺=215.0. ¹H NMR (400 MHz, CHLOROFORM-d) δ=6.49 (s, 1H), 4.08-4.02 (m, 4H), 2.91 (t, J=6.4 Hz, 2H), 2.74 (s, 2H), 1.99 (t, J=6.5 Hz, 2H)

### Procedure for preparation of 3-fluoro-6,7-dihydro-4H-benzothiophen-5-one (7)

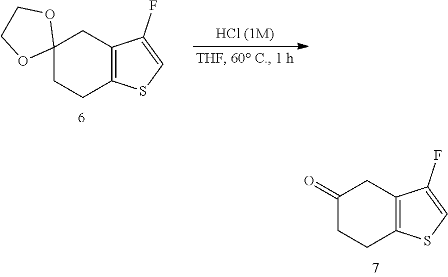 [SMILES: Fc1csc2c1CC1(CC2)OCCO1 ; O=C1CCc2scc(F)c2C1]

To a solution of 3′-fluorospiro[1,3-dioxolane-2,5′-6,7-dihydro-4H-benzothiophene](6) (1.2 g, 5.60 mmol, 1 eq) in THF (30 mL) was added HCl (1 M aq., 30 mL, 5.36 eq). The mixture was stirred at 60° C. for 1 hr. The reaction mixture was diluted with aq. sat. NaHCO₃ (20 mL) and extracted with EtOAc (30 mL*2). The combined organic layers were washed with aq. sat. NaCl (20 mL), dried over anhydrous Na₂SO₄, filtered and concentrated under reduced pressure to give a residue. Compound 3-fluoro-6,7-dihydro-4H-benzothiophen-5-one (7) (900 mg, 5.29 mmol, 94.41% yield) was obtained as a yellow oil. LCMS: m/z [M+H]⁺=171.0. ¹H NMR (400 MHz, CHLOROFORM-d) 8=6.57 (s, 1H), 3.39 (s, 2H), 3.11 (t, J=6.8 Hz, 2H), 2.72 (t, J=6.8 Hz, 2H)

### Procedure for preparation of 3-fluoro-6,7-dihydro-4H-benzothiophen-5-one oxime (8)

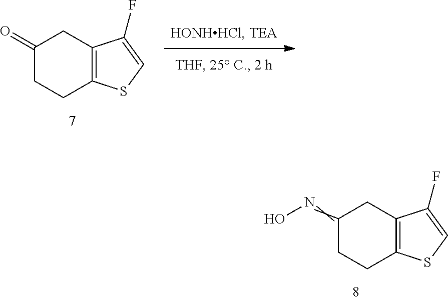 [SMILES: O=C1CCc2scc(F)c2C1 ; ON=C1CCc2scc(F)c2C1]

To a solution of 3-fluoro-6,7-dihydro-4H-benzothiophen-5-one (7) (900 mg, 5.29 mmol, 1 eq) in THF (5 mL) and H₂O (5 mL) was added NH₂OH·HCl (734.91 mg, 10.58 mmol, 2 eq) and TEA (1.61 g, 15.86 mmol, 2.21 mL, 3 eq). The mixture was stirred at 25° C. for 2 hr. The reaction mixture was diluted with H₂O (10 mL) and extracted with EtOAc (20 mL*2). The combined organic layers were washed with aq. sat. NaCl (10 mL), dried over anhydrous Na₂SO₄, filtered and concentrated under reduced pressure to give a residue. Compound 3-fluoro-6,7-dihydro-4H-benzothiophen-5-one oxime (8) (900 mg, crude) was obtained as a yellow solid. LCMS: m/z [M+H]⁺=185.8

### Procedure for preparation of 3-fluoro-4,5,6,7-tetrahydrobenzothiophen-5-amine (9)

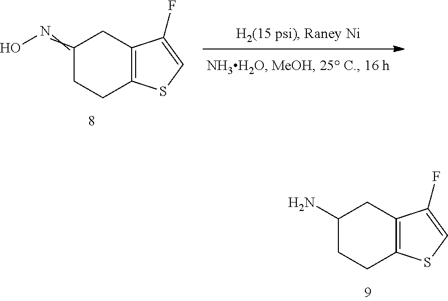 [SMILES: ON=C1CCc2scc(F)c2C1 ; NC1CCc2scc(F)c2C1]

To a solution of 3-fluoro-6,7-dihydro-4H-benzothiophen-5-one oxime (8) (1.05 g, 5.67 mmol, 1 eq) and NH₃·H₂O (91.00 mg, 1.04 mmol, 0.1 mL, 40% aq) in MeOH (15 mL) was added Raney-Ni (400 mg) under N₂. The suspension was degassed under vacuum and purged with H₂ several times. The mixture was stirred under H₂ (15 psi) at 25° C. for 16 hours. The reaction solution is diluted with methanol (10 ml) and the supernatant was removed and collected. This process was repeated 3 times. Combined solvent layer was evaporated. Compound 3-fluoro-4,5,6,7-tetrahydrobenzothiophen-5-amine (9) (1 g, crude) was obtained as a yellow solid. LCMS: m/z [M+H]⁺=171.9

### Procedure for preparation of tert-butyl N-(3-fluoro-4,5,6,7-tetrahydrobenzothiophen-5-yl)carbamate (10)

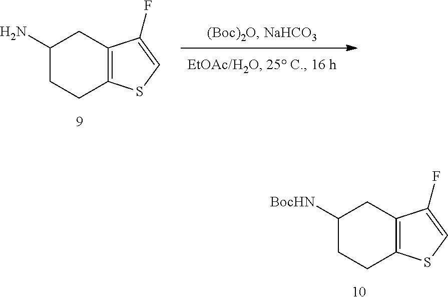 [SMILES: NC1CCc2scc(F)c2C1 ; CC(C)(C)OC(=O)NC1CCc2scc(F)c2C1]

To a solution of 3-fluoro-4,5,6,7-tetrahydrobenzothiophen-5-amine (9) (1 g, 5.84 mmol, 1 eq) and (Boc)₂O (2.55 g, 11.68 mmol, 2.68 mL, 2 eq) in EtOAc (10 mL) and H₂O (5 mL) was added NaHCO₃ (981.19 mg, 11.68 mmol, 2 eq). The mixture was stirred at 25° C. for 16 hr. The reaction mixture was diluted with H₂O (10 mL) and extracted with EtOAc (30 mL*2). The combined organic layers were washed with aq. sat. NaCl (10 mL), dried over anhydrous Na₂SO₄, filtered and concentrated under reduced pressure to give a residue. The residue was purified by flash silica gel chromatography (ISCO; 40 g SepaFlash Silica Flash Column, eluent 0~20% Ethyl acetate/Petroleum ether gradient @40 mL/min) and then with prep-HPLC (column: Phenomenex luna C18 150*25 mm*10 μm; mobile phase: [water(FA)-ACN]; gradient:55%-85% B over 7 min). Compound tert-butyl N-(3-fluoro-4,5,6,7-tetrahydrobenzothiophen-5-yl)carbamate (10) (300 mg, 1.11 mmol, 18.93% yield) was obtained as a yellow solid. LCMS: m/z [M+H-tBu]⁺=216.2. ¹H NMR (400 MHz, CHLOROFORM-d) δ=6.48 (s, 1H), 4.70-4.52 (m, 1H), 4.08-3.97 (m, 1H), 2.95-2.76 (m, 3H), 2.36 (br dd, J=7.3, 16.4 Hz, 1H), 2.12-2.01 (m, 1H), 1.92-1.79 (m, 1H), 1.57 (d, J=2.4 Hz, 1H), 1.47 (s, 9H)

### Procedure for preparation of tert-butyl N-(3-fluoro-4,5,6,7-tetrahydrobenzothiophen-5-yl)carbamate enantiomer 1 (10a) and enantiomer 2 (10b)

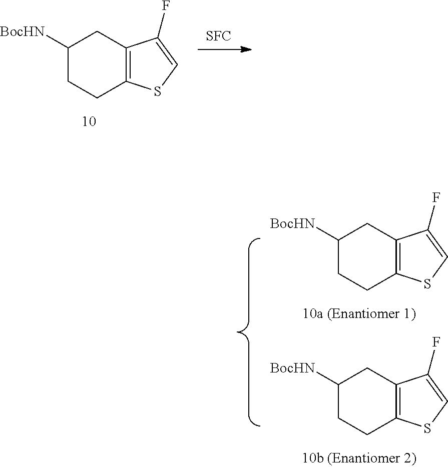

Racemic tert-butyl N-(3-fluoro-4,5,6,7-tetrahydrobenzothiophen-5-yl)carbamate (10) was separated to enantiomers by SFC (column: DAICEL CHIRALPAK IG (250 mm*30 mm, 10 um); mobile phase: [CO₂-MeOH (0.1% NH₃H₂O)]; B %:12%, isocratic elution mode). The 1^{st} enantiomer 10a (RT=1.018 min, 67 mg) and the 2nd enantiomer 10b (RT=1.262 min, 70 mg) were isolated both as yellow solids. Compound tert-butyl N-(3-fluoro-4,5,6,7-tetrahydrobenzothiophen-5-yl)carbamate enantiomer 1 (10a) (67 mg, 246.91 μmol, 44.67% yield). Compound tert-butyl N-(3-fluoro-4,5,6,7-tetrahydrobenzothiophen-5-yl)carbamate enantiomer 2 (10b) (70 mg, 257.97 μmol, 46.67% yield). LCMS: m/z [M+H-tBu]⁺=216.2.

### Procedure for preparation of 3-fluoro-4,5,6,7-tetrahydrobenzothiophen-5-amine hydrochloride enantiomer 1 (191)

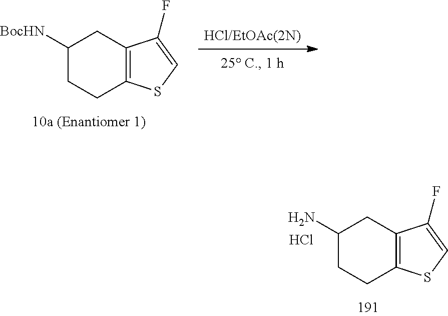 [SMILES: CC(C)(C)OC(=O)NC1CCc2scc(F)c2C1 ; "25° C., 1 h" ; "HCl/EtOAc(2N)" ; Cl ; NC1CCc2scc(F)c2C1]

To a solution of tert-butyl N-(3-fluoro-4,5,6,7-tetrahydrobenzothiophen-5-yl)carbamate enantiomer 1 (10a) (67 mg, 246.91 μmol, 1 eq) in EtOAc (2 mL) was added HCl/EtOAc (4 M, 2 mL, 32.40 eq). The mixture was stirred at 25° C. for 1 hr. The reaction mixture was filtered and the cake was washed with EtOAc (10 mL). Compound 3-fluoro-4,5,6,7-tetrahydrobenzothiophen-5-amine hydrochloride enantiomer 1 (191) (41.28 mg, 193.94 μmol, 78.55% yield) was obtained as a white solid. ¹H NMR (400 MHz, METHANOL-d4) δ=6.73 (s, 1H), 3.66-3.55 (m, 1H), 3.05-2.96 (m, 1H), 2.91 (br t, J=5.4 Hz, 2H), 2.60-2.48 (m, 1H), 2.31-2.20 (m, 1H), 2.05-1.89 (m, 1H). LCMS: m/z [M+H]⁺=172.1

### Procedure for preparation of 3-fluoro-4,5,6,7-tetrahydrobenzothiophen-hydrochloride enantiomer 2 (192)

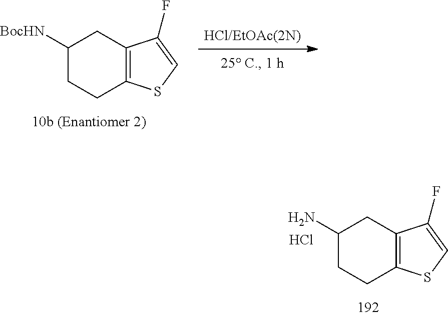 [SMILES: CC(C)(C)OC(=O)NC1CCc2scc(F)c2C1 ; "25° C., 1 h" ; "HCl/EtOAc(2N)" ; Cl ; NC1CCc2scc(F)c2C1]

To a solution of tert-butyl N-(3-fluoro-4,5,6,7-tetrahydrobenzothiophen-5-yl)carbamate enantiomer 2 (10b) (70 mg, 257.97 μmol, 1 eq) in EtOAc (2 mL) was added HCl/EtOAc (4 M, 2 mL, 31.01 eq). The mixture was stirred at 25° C. for 1 hr. The reaction mixture was filtered and the cake was washed with EtOAc (10 mL). Compound 3-fluoro-4,5,6,7-tetrahydrobenzothiophen-5-amine hydrochloride enantiomer 2 (192) (38.53 mg, 184.32 μmol, 71.45% yield) was obtained as a white solid. ¹H NMR (400 MHz, METHANOL-d4) δ=6.72 (s, 1H), 3.65-3.56 (m, 1H), 3.00 (br dd, J=4.9, 15.9 Hz, 1H), 2.91 (br t, J=5.9 Hz, 2H), 2.60-2.48 (m, 1H), 2.26 (br d, J=13.0 Hz, 1H), 2.03-1.89 (m, 1H). LCMS: m/z [M+H]⁺=172.1

### Synthetic scheme for preparation of 3-cyclopropyl-4,5,6,7-tetrahydro-2-benzothiophen-5-amine hydrochloride (193)

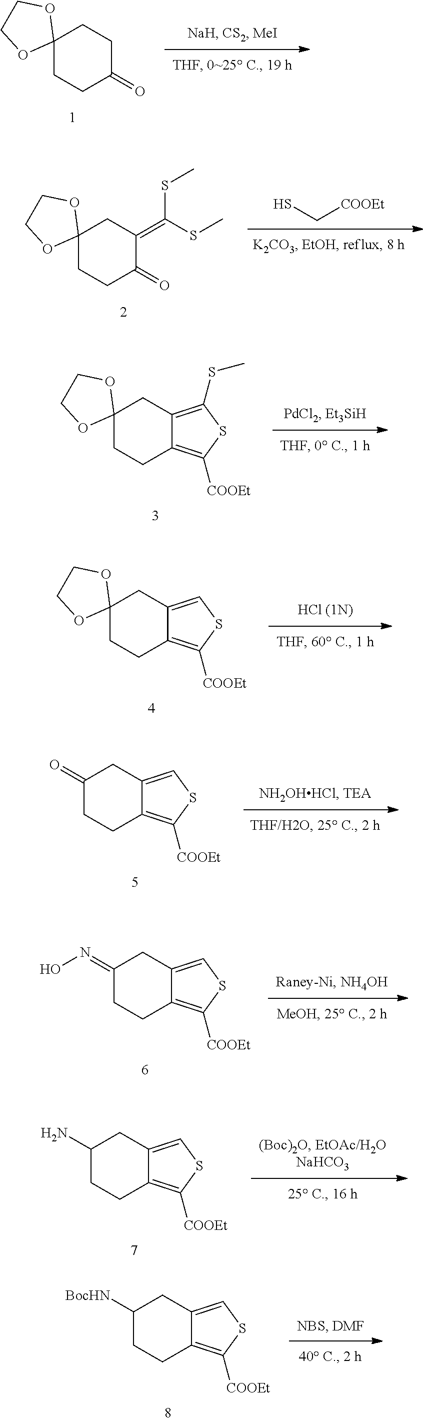

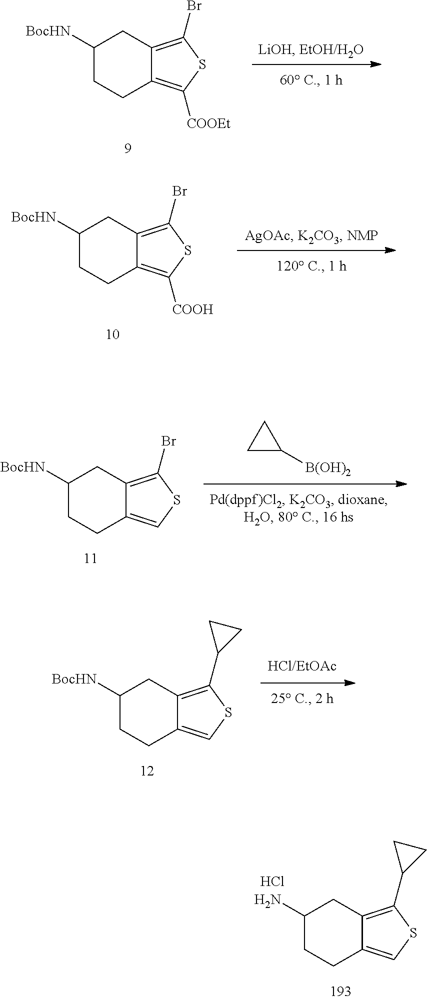

### Procedure for preparation of 7-[bis(methylsulfanyl)methylene]-1,4-dioxaspiro[4.5]decan-8-one (2)

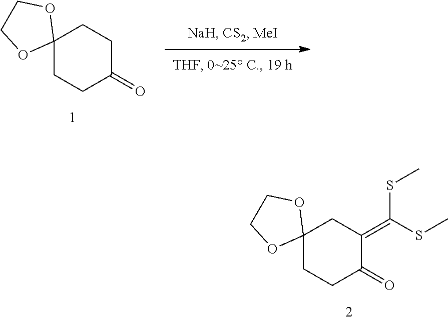 [SMILES: O=C1CCC2(CC1)OCCO2 ; CSC(SC)=C1CC2(CCC1=O)OCCO2]

NaH (28.17 g, 704.32 mmol, 60% suspension in mineral oil, 1.1 eq) was added slowly to THF (1 L) in three round bottom flask (3 L) under N₂. The reaction mixture was cooled to 0° C., and then 1,4-dioxaspiro[4.5]decan-8-one (1) (100 g, 640.29 mmol, 1 eq) was added slowly under N₂. The mixture was stirred at 0° C. for 0.5 h under N₂, then a solution of CS₂ (51.19 g, 672.31 mmol, 40.53 mL, 1.05 eq) in THF (0.5 L) was added dropwise under N₂. The mixture was stirred at 0° C. for 0.5 h, a solution of iodomethane (209.03 g, 1.47 μmol, 91.68 mL, 2.3 eq) in THF (0.5 L) was then added dropwise at 0° C. and the mixture was stirred at 0° C. for 2 h and then at 25° C. for 16 h. The reaction mixture was then added to sat.NH₄Cl (1 L) at 0° C., and diluted with H₂O 500 mL and the resulting mixture was extracted with EtOAc (800 mL*2). The combined organic layers were washed with brine (800 mL), dried over anhydrous Na₂SO₄, filtered and concentrated under reduced pressure to give a residue. The residue was purified by flash silica gel chromatography (ISCO®; 330 g SepaFlash® Silica Flash Column, eluent 0~20% Ethylacetate/Petroleum ether gradient @85 mL/min). Compound 7-[bis(methylsulfanyl)methylene]-1,4-dioxaspiro[4.5]decan-8-one (2) (60 g, 230.44 mmol, 35.99% yield) was obtained as a yellow solid. LCMS: m/z [M+H]+=261.1.

### Procedure for preparation of ethyl 3′-methylsulfanylspiro[1,3-dioxolane-2,5′-6,7-dihydro-4H-2-benzothiophene]-1′-carboxylate (3)

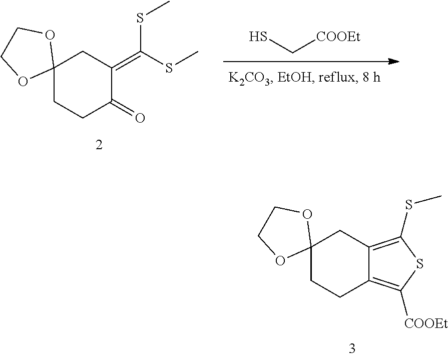 [SMILES: CSC(SC)=C1CC2(CCC1=O)OCCO2 ; CCOC(=O)CS ; CCOC(=O)c1sc(SC)c2c1CCC1(C2)OCCO1]

To a mixture of 7-[bis(methylsulfanyl)methylene]-1,4-dioxaspiro[4.5]decan-8-one (2) (60 g, 230.44 mmol, 1 eq) and K₂CO₃ (38.22 g, 276.53 mmol, 1.2 eq) in EtOH (1 L) was added ethyl 2-sulfanylacetate (30.38 g, 252.81 mmol, 27.62 mL, 1.10 eq) under N₂. The mixture was stirred at 80° C. for 8 hours. The mixture was evaporated and the residue was treated with water and extracted with EtOAc (100 mL*3). The combined organic phases washed with brine (100 mL*3), dried over Na₂SO₄, filtered and concentrated at reduced pressure. The residue was purified by flash silica gel chromatography (ISCO®; 220 g SepaFlash® Silica Flash Column, eluent 0~20% Ethylacetate/Petroleum ether gradient @65 mL/min). Compound ethyl 3′-methylsulfanylspiro[1,3-dioxolane-2,5′-6,7-dihydro-4H-2-benzothiophene]-1′-carboxylate (3) (40.7 g, 129.44 mmol, 56.17% yield) was obtained as a yellow solid. LCMS: m/z [M+H]⁺=314.8.

### Procedure for preparation of ethyl spiro[1,3-dioxolane-2,5′-6,7-dihydro-4H-2-benzothiophene]-1′-carboxylate (4)

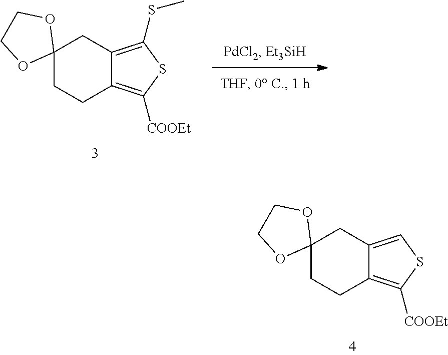 [SMILES: CCOC(=O)c1sc(SC)c2c1CCC1(C2)OCCO1 ; CCOC(=O)c1scc2c1CCC1(C2)OCCO1]

To a solution of ethyl 3′-methylsulfanylspiro[1,3-dioxolane-2,5′-6,7-dihydro-4H-2-benzothiophene]-1′-carboxylate (3) (40.7 g, 129.44 mmol, 1 eq) in THF (500 mL) was added PdCl₂ (2.30 g, 12.94 mmol, 0.1 eq) and Et₃SiH (30.10 g, 258.89 mmol, 41.35 mL, 2 eq) at 0° C. The mixture was stirred at 0° C. for 1 hr. The mixture was diluted with H₂O (500 mL), and extracted with EtOAc (200 mL*3). The combined organic phase was washed with brine (200 mL*3), dried over Na₂SO₄, filtered and concentrated at reduced pressure. The residue was purified by flash silica gel chromatography (ISCO®; 120 g SepaFlash® Silica Flash Column, eluent 0~20% Ethyl acetate/Petroleum ether gradient @65 mL/min). Compound ethyl spiro[1,3-dioxolane-2,5′-6,7-dihydro-4H-2-benzothiophene]-1′-carboxylate (4) (28 g, 104.35 mmol, 80.61% yield) was obtained as a yellow gum. LCMS: m/z [M+H]+=269.0.

### Procedure for preparation of ethyl 5-oxo-6,7-dihydro-4H-2-benzothiophene-1-carboxylate (5)

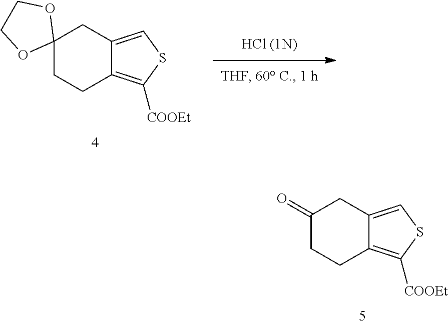 [SMILES: CCOC(=O)c1scc2c1CCC1(C2)OCCO1 ; CCOC(=O)c1scc2c1CCC(=O)C2]

To a solution of ethyl spiro[1,3-dioxolane-2,5′-6,7-dihydro-4H-2-benzothiophene]-1′-carboxylate (4) (14 g, 52.17 mmol, 1 eq) in THF (100 mL) was added HCl (1 M, 100 mL, 1.92 eq). The mixture was stirred at 60° C. for 1 hr. The mixture was extracted with EtOAc (200 mL*3). The combined organic phases were washed with brine (100 mL*3), dried over Na₂SO₄, filtered and concentrated at reduced pressure to give a residue. The crude product ethyl 5-oxo-6,7-dihydro-4H-2-benzothiophene-1-carboxylate (5) (10.2 g, 45.48 mmol, 87.17% yield) was obtained as a yellow gum and used into the next step without further purification. LCMS: m/z [M+H]⁺=225.0.

### Procedure for preparation of ethyl 5-hydroxyimino-6,7-dihydro-4H-2-benzothiophene-1-carboxylate (6)

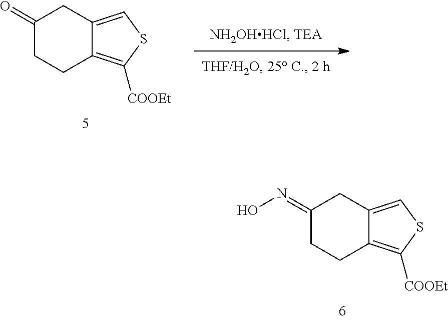 [SMILES: CCOC(=O)c1scc2c1CCC(=O)C2 ; CCOC(=O)c1scc2c1CC/C(=N\O)C2]

To a solution of ethyl 5-oxo-6,7-dihydro-4H-2-benzothiophene-1-carboxylate (5) (10.2 g, 45.48 mmol, 1 eq) and TEA (13.81 g, 136.44 mmol, 18.99 mL, 3 eq) in THF (100 mL) was added NH₂OH·HCl (6.32 g, 90.96 mmol, 2 eq) in H₂O (100 mL). The mixture was stirred at 25° C. for 2 hr. The reaction mixture was diluted with H₂O 100 mL and extracted with EtOAc (300 mL*2). The combined organic layers were washed with aq. sat.NaCl (100 mL), dried over anhydrous Na₂SO₄, filtered and concentrated under reduced pressure. The residue was purified by flash silica gel chromatography (ISCO®; 80 g SepaFlash® Silica Flash Column, eluent 0~50% Ethyl acetate/Petroleum ether gradient @65 mL/min). Compound ethyl 5-hydroxyimino-6,7-dihydro-4H-2-benzothiophene-1-carboxylate (6) (9.6 g, 40.12 mmol, 88.21% yield) was obtained as a yellow solid. LCMS: m/z [M+H]⁺=240.2.

### Procedure for preparation of ethyl 5-amino-4,5,6,7-tetrahydro-2-benzothiophene-1-carboxylate (7)

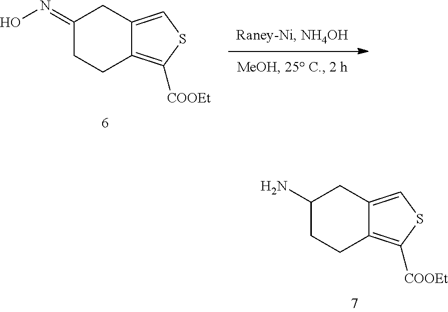 [SMILES: CCOC(=O)c1scc2c1CC/C(=N\O)C2 ; CCOC(=O)c1scc2c1CCC(N)C2]

To a solution of ethyl 5-hydroxyimino-6,7-dihydro-4H-2-benzothiophene-1-carboxylate (6) (9.6 g, 40.12 mmol, 1 eq) and NH₃·H₂O (9.10 g, 72.70 mmol, 10 mL, 28%, 1.81 eq) in MeOH (100 mL) was added Raney-Ni (3.44 g, 40.12 mmol, 1 eq) under N₂. The suspension was degassed under vacuum and purged with H₂ several times. The mixture was stirred under H₂ (15 psi) at 25° C. for 2 hours, filtered to remove insolubles and concentrated under reduced pressure to give the crude product. The crude product ethyl 5-amino-4,5,6,7-tetrahydro-2-benzothiophene-1-carboxylate (7) (9 g, 39.95 mmol, 99.57% yield) was obtained as a yellow solid and used into the next step without further purification. LCMS: m/z [M+H]⁺=226.2.

### Procedure for preparation of ethyl 5-(tert-butoxycarbonylamino)-4,5,6,7-tetrahydro-2-benzothiophene-1-carboxylate (8)

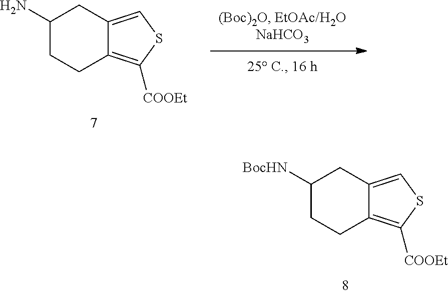 [SMILES: CCOC(=O)c1scc2c1CCC(N)C2 ; CCOC(=O)c1scc2c1CCC(NC(=O)OC(C)(C)C)C2]

To a solution of ethyl 5-amino-4,5,6,7-tetrahydro-2-benzothiophene-1-carboxylate (7) (9 g, 39.95 mmol, 1 eq) in EtOAc (100 mL) and H₂O (100 mL) was added (Boc)₂O (13.08 g, 59.92 mmol, 13.77 mL, 1.5 eq) and NaHCO₃ (10.07 g, 119.84 mmol, 4.66 mL, 3 eq). The mixture was stirred at 25° C. for 16 hr. The mixture was extracted with EtOAc (100 mL*3). The combined organic phases were washed with brine (100 mL), dried over Na₂SO₄, filtered and concentrated at reduced pressure. The residue was purified by flash silica gel chromatography (ISCO®; 80 g SepaFlash® Silica Flash Column, eluent 0-30% Ethyl acetate/Petroleum ether gradient @45 mL/min). Compound ethyl 5-(tert-butoxycarbonylamino)-4,5,6,7-tetrahydro-2-benzothiophene-1-carboxylate (8) (7.1 g, 21.82 mmol, 54.62% yield) was obtained as a white solid. LCMS: m/z [M-Boc+H]⁺=224.1.

### Procedure for preparation of ethyl 3-bromo-5-(tert-butoxycarbonylamino)-4,5,6,7-tetrahydro-2-benzothiophene-1-carboxylate (9)

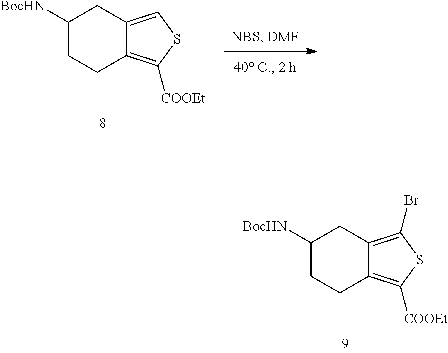 [SMILES: CCOC(=O)c1scc2c1CCC(NC(=O)OC(C)(C)C)C2 ; CCOC(=O)c1sc(Br)c2c1CCC(NC(=O)OC(C)(C)C)C2]

To a solution of ethyl 5-(tert-butoxycarbonylamino)-4,5,6,7-tetrahydro-2-benzothiophene-1-carboxylate (8) (2.5 g, 7.68 mmol, 1 eq) in DMF (10 mL) was added NBS (1.64 g, 9.22 mmol, 1.2 eq). The mixture was stirred at 40° C. for 2 hr. The mixture was then quenched with aq sat. Na₂SO₃ (40 mL) diluted with H₂O (20 mL) and extracted with EtOAc (50 mL*3). The combined organic layers were washed with brine (20 mL), dried over Na₂SO₄, filtered and concentrated under reduced pressure. The residue was purified by flash silica gel chromatography (ISCO®; 40 g SepaFlash® Silica Flash Column, eluent 0-30% Ethyl acetate/Petroleum ether gradient @35 mL/min). Compound ethyl 3-bromo-5-(tert-butoxycarbonylamino)-4,5,6,7-tetrahydro-2-benzothiophene-1-carboxylate (9) (2.4 g, 5.94 mmol, 77.27% yield) was obtained as a yellow solid. LCMS: m/z [M-Boc+H]⁺=303.7.

### Procedure for preparation of 3-bromo-5-(tert-butoxycarbonylamino)-4,5,6,7-tetrahydro-2-benzothiophene-1-carboxylic acid (10)

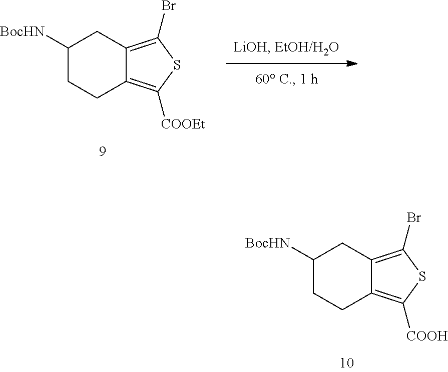 [SMILES: CCOC(=O)c1sc(Br)c2c1CCC(NC(=O)OC(C)(C)C)C2 ; "LiOH, EtOH/H2O" ; CC(C)(C)OC(=O)NC1CCc2c(C(=O)O)sc(Br)c2C1]

To a solution of ethyl 3-bromo-5-(tert-butoxycarbonylamino)-4,5,6,7-tetrahydro-2-benzothiophene-1-carboxylate (9) (2.8 g, 6.93 mmol, 1 eq) in EtOH (20 mL) was added LiOH·H₂O (581.22 mg, 13.85 mmol, 2 eq) in H₂O (5 mL). The mixture was stirred at 60° C. for 1 hr and then concentrated under reduced pressure to remove EtOH. The residue was diluted with H₂O (100 mL) and acidified to pH=6 with HCl (aq. 1 M). The mixture was extracted with EtOAc (100 mL*3). The combined organic phase was washed with brine (100 mL), dried over Na₂SO₄, filtered and concentrated at reduced pressure to give a residue. The crude product 3-bromo-5-(tert-butoxycarbonylamino)-4,5,6,7-tetrahydro-2-benzothiophene-1-carboxylic acid (10) (2.1 g, 5.58 mmol, 80.59% yield) was obtained as a white solid and used into the next step without further purification. LCMS: m/z [M-Boc+H]⁺=277.7.

### Procedure for preparation of tert-butyl N-(3-bromo-4,5,6,7-tetrahydro-2-benzothiophen-5-yl)carbamate (11)

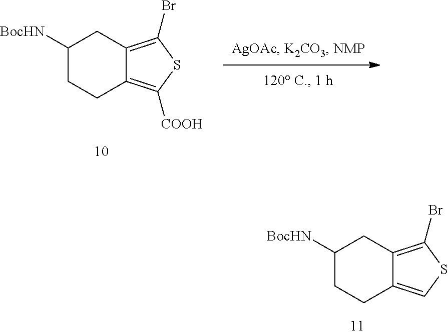

To a solution of 3-bromo-5-(tert-butoxycarbonylamino)-4,5,6,7-tetrahydro-2-benzothiophene-1-carboxylic acid (10) (2.1 g, 5.58 mmol, 1 eq) in NMP (10 mL) was added AgOAc (93.16 mg, 28.58 μL, 0.1 eq) and K₂CO₃ (2.31 g, 16.74 mmol, 3 eq). The mixture was stirred at 120° C. for 1 hr. The mixture was diluted with H₂O (100 mL) and extracted with EtOAc (100 mL*3). The combined organic phase was washed with brine (100 mL*3), dried over Na₂SO₄, filtered and concentrated at reduced pressure. The residue was purified by flash silica gel chromatography (ISCO®; 80 g SepaFlash® Silica Flash Column, eluent 0~25% Ethylacetate/Petroleum ether gradient @65 mL/min). Compound tert-butyl N-(3-bromo-4,5,6,7-tetrahydro-2-benzothiophen-5-yl)carbamate (11) (1.8 g, 5.42 mmol, 97.07% yield) was obtained as a white solid. LCMS: m/z [M-t-Bu+H]⁺=277.7.

### Procedure for preparation of tert-butyl N-(3-cyclopropyl-4,5,6,7-tetrahydro-2-benzothiophen-5-yl)carbamate (12)

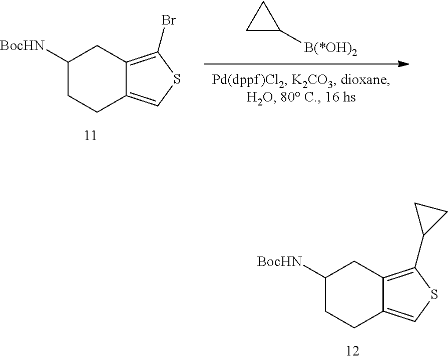

To a solution of tert-butyl N-(3-bromo-4,5,6,7-tetrahydro-2-benzothiophen-5-yl)carbamate (11) (200 mg, 601.95 μmol, 1 eq), cyclopropylboronic acid (129.26 mg, 1.50 mmol, 2.5 eq) and K₂CO₃ (249.58 mg, 1.81 mmol, 3 eq) in dioxane (1 mL) and H₂O (0.1 mL) was added Pd(dppf)Cl₂ (44.04 mg, 60.19 μmol, 0.1 eq) under N₂. The mixture was stirred at 80° C. for 16 hr. Then the reaction mixture was concentrated under reduced pressure to give a residue. The residue was purified by flash silica gel chromatography (ISCO®; 7 g SepaFlash® Silica Flash Column, eluent 0~25% Ethylacetate/Petroleum ether gradient @25 mL/min). Compound tert-butyl N-(3-cyclopropyl-4,5,6,7-tetrahydro-2-benzothiophen-5-yl)carbamate (12) (80 mg, 272.64 μmol, 45.29% yield) was obtained as a white solid. LCMS: m/z [M+H]⁺=294.0.

### Procedure for preparation of 3-cyclopropyl-4,5,6,7-tetrahydro-2-benzothiophen-5-amine hydrochloride (193)

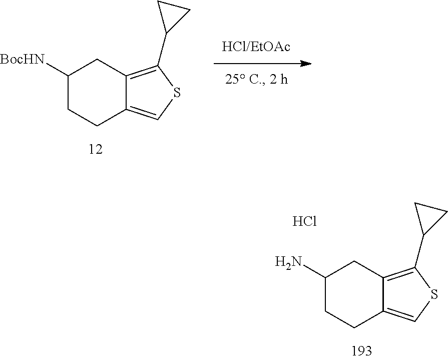 [SMILES: CC(C)(C)OC(=O)NC1CCc2csc(C3CC3)c2C1 ; NC1CCc2csc(C3CC3)c2C1 ; Cl]

To a solution of tert-butyl N-(3-cyclopropyl-4,5,6,7-tetrahydro-2-benzothiophen-5-yl)carbamate (12) (80 mg, 272.64 μmol, 1 eq) in EtOAc (1 mL) was added HCl/EtOAc (4 M, 1 mL, 14.67 eq). The mixture was stirred at 25° C. for 2 hr. The reaction mixture was concentrated under reduced pressure to give a residue. The residue was purified by prep-HPLC (column: Phenomenex luna C18 150*25 mm*10 μm; mobile phase: [water(HCl)-ACN]; gradient:5%-35% B over 10 min). Compound 3-cyclopropyl-4,5,6,7-tetrahydro-2-benzothiophen-5-amine hydrochloride (193) (33.7 mg, 146.67 μmol, 53.80% yield) was obtained as a white solid. LCMS: m/z [M+H]⁺=194.2. ¹H NMR (400 MHz, METHANOL-d₄) δ=6.72 (s, 1H), 3.59-3.48 (m, 1H), 3.30-3.23 (m, 1H), 2.96-2.86 (m, 1H), 2.81-2.70 (m, 1H), 2.65 (dd, J=9.9, 15.7 Hz, 1H), 2.20-2.11 (m, 1H), 1.99-1.89 (m, 1H), 1.82 (dtd, J=5.5, 11.1, 12.6 Hz, 1H), 1.02-0.96 (m, 2H), 0.66-0.59 (m, 2H)

### Synthetic scheme for preparation of N-methyl-3-(trifluoromethyl)-4,5,6,7-tetrahydrobenzothiophen-5-amine hydrochloride enantiomer 1 (194) and enantiomer 2 (195)

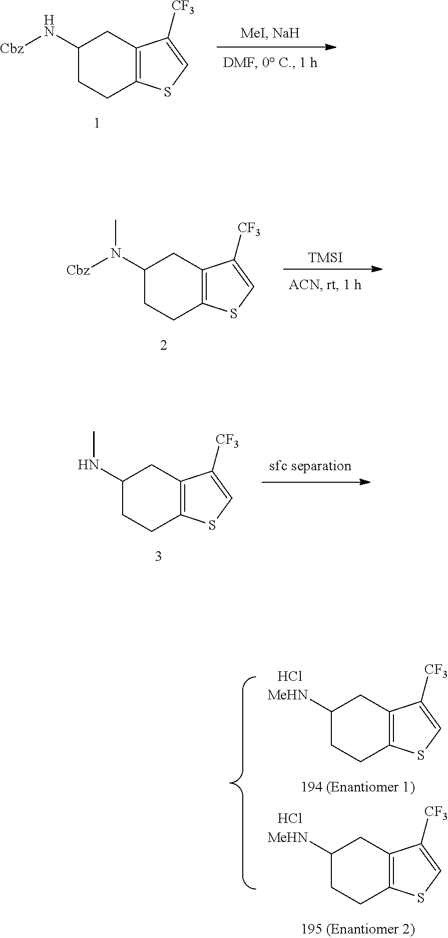

### Procedure for preparation of benzyl N-methyl-N-[3-(trifluoromethyl)-4,5,6,7-tetrahydrobenzothiophen-5-yl]carbamate (2)

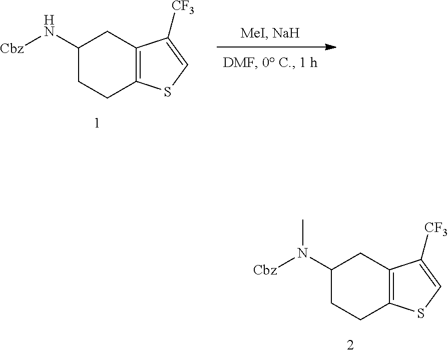

To a solution of benzyl N-[3-(trifluoromethyl)-4,5,6,7-tetrahydrobenzothiophen-5-yl]carbamate (1) (450 mg, 1.27 mmol, 1 eq) in DMF (10 mL) was added NaH (65.84 mg, 1.65 mmol, 60% suspension in mineral oil, 1.3 eq) at 0° C. Mixture stirred for 0.5 h, then MeI (233.65 mg, 1.65 mmol, 102.48 μL, 1.3 eq) was added. The mixture was stirred at 0° C. for 0.5 h. The reaction mixture was then diluted with aq. sat.NH₄Cl (50 mL) and extracted with EtOAc (30 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~8% Ethyl acetate/Petroleum ether gradient @40 mL/min). Compound benzyl N-methyl-N-[3-(trifluoromethyl)-4,5,6,7-tetrahydrobenzothiophen-5-yl]carbamate (2) (440 mg, 1.19 mmol, 94.07% yield) was obtained as a colorless oil. ¹H NMR (400 MHz, CHLOROFORM-d) δ 7.52 (s, 1H), 7.38 (br s, 5H), 5.23-5.11 (m, 2H), 4.66-4.29 (m, 1H), 3.03-2.82 (m, 6H), 2.79-2.64 (m, 1H), 2.03-1.91 (m, 2H).LCMS: m/z [M+H]⁺=369.9.

### Procedure for preparation of N-methyl-3-(trifluoromethyl)-4,5,6,7-tetrahydrobenzothiophen-5-amine (3)

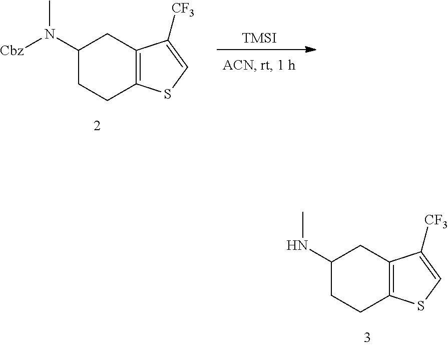 [SMILES: "TMSI" ; CN(C(=O)OCc1ccccc1)C1CCc2scc(C(F)(F)F)c2C1 ; CNC1CCc2scc(C(F)(F)F)c2C1]

To a solution of benzyl N-methyl-N-[3-(trifluoromethyl)-4,5,6,7-tetrahydrobenzothiophen-5-yl]carbamate (2) (440 mg, 1.19 mmol, 1 eq) in ACN (5 mL) was added TMSI (953.34 mg, 4.76 mmol, 648.53 μL, 4 eq). The mixture was stirred at 20° C. for 1 hr. The reaction mixture was diluted with H₂O (10 mL) and extracted with EtOAc (30 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~100% Ethyl acetate/Petroleum ether gradient @40 mL/min). Compound N-methyl-3-(trifluoromethyl)-4,5,6,7-tetrahydrobenzothiophen-5-amine (3) (200 mg, 850.09 μmol, 71.37% yield) was obtained as a yellow oil. LCMS: m/z [M+H]⁺=236.2

### Procedure for preparation of N-methyl-3-(trifluoromethyl)-4,5,6,7-tetrahydrobenzothiophen-5-amine hydrochloride enantiomer 1 (194) and enantiomer 2 (195)

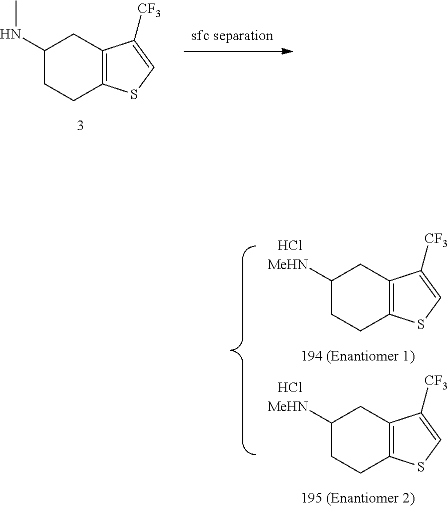

Racemic N-methyl-3-(trifluoromethyl)-4,5,6,7-tetrahydrobenzothiophen-5-amine (3) was separated to its enantiomers by SFC (column: DAICEL CHIRALPAK AS (250 mm*50 mm, 10 μm); mobile phase: [CO₂-EtOH (0.1% NH₃H₂O)]; B %:15%, isocratic elution mode). The 1^{st} enantiomer (RT=0.696 min) and the 2nd enantiomer (RT=0.818 min), were both obtained as colorless oil. Each enantiomer was dissolved in EtOAc (2 ml) and to the mixture was added HCl/EtOAc (4N, 2 ml). The solid formed was filtered and the cake was washed with EtOAc (10 mL). Compound N-methyl-3-(trifluoromethyl)-4,5,6,7-tetrahydrobenzothiophen-5-amine hydrochloride enantiomer 1 (194) (76.03 mg, 279.80 μmol, 32.91% yield) was obtained as a white solid. ¹H NMR (400 MHz, METHANOL-d4) δ 7.85 (s, 1H), 3.62-3.54 (m, 1H), 3.29-3.21 (m, 1H), 3.10-2.91 (m, 2H), 2.86-2.71 (m, 4H), 2.43-2.32 (m, 1H), 2.06-1.91 (m, 1H). LCMS: m/z [M+H]⁺=236.2. Compound N-methyl-3-(trifluoromethyl)-4,5,6,7-tetrahydrobenzothiophen-5-amine hydrochloride enantiomer 2 (195) (66.39 mg, 244.32 μmol, 28.74% yield) was also obtained as a white solid. ¹H NMR (400 MHz, METHANOL-d4) δ 7.85 (s, 1H), 3.62-3.54 (m, 1H), 3.25 (br dd, J=5.0, 16.0 Hz, 1H), 3.09-2.92 (m, 2H), 2.85-2.71 (m, 4H), 2.42-2.32 (m, 1H), 2.03-1.91 (m, 1H). LCMS: m/z [M+H]⁺=236.2.

### Synthetic scheme for preparation of 3-fluoro-N, 2-dimethyl-4,5,6,7-tetrahydrobenzothiophen-5-amine hydrochloride (196)

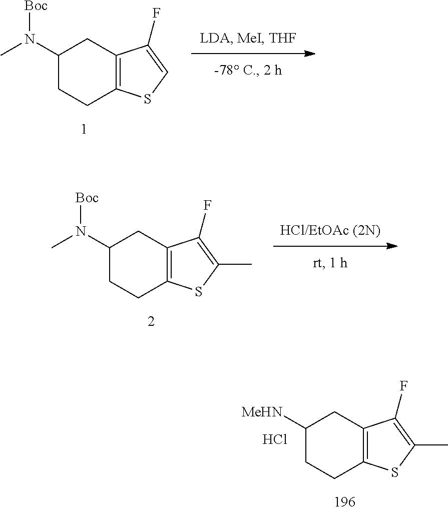

### Procedure for tert-butyl N-(3-fluoro-2-methyl-4,5,6,7-tetrahydrobenzothiophen-5-yl)-N-methyl-carbamate (2)

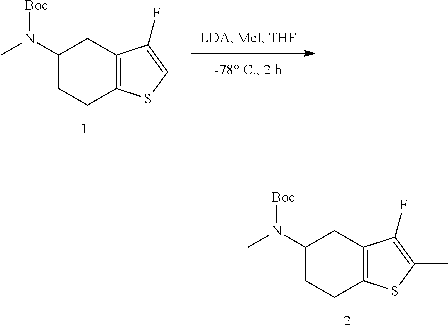

To a solution of tert-butyl N-(3-fluoro-4,5,6,7-tetrahydrobenzothiophen-5-yl)-N-methyl-carbamate (1) (300 mg, 1.05 mmol, 1 eq) in THF (10 mL) was added dropwise LDA (2 M, 630.75 μL, 1.2 eq) at −78° C. After addition, the mixture was stirred at −78° C. for 1 hr, and then CH₃I (179.05 mg, 1.26 mmol, 78.53 μL, 1.2 eq) was added dropwise at −78° C. The resulting mixture was stirred at −78° C. for 1 hr. The reaction mixture was diluted with H₂O (10 mL) and extracted with EtOAc (10 mL*2). The combined organic layers were washed with aq. sat.NaCl (10 mL), dried over anhydrous Na₂SO₄, filtered and concentrated under reduced pressure to give a residue. The residue was purified by prep-HPLC (column: Phenomenex luna C18 150*25 mm*10 μm; mobile phase: [water(FA)-ACN]; gradient:70%-100% B over 7 min). Compound tert-butyl N-(3-fluoro-2-methyl-4,5,6,7-tetrahydrobenzothiophen-5-yl)-N-methyl-carbamate (2) (280 mg, 935.19 μmol, 88.96% yield) was obtained as a yellow oil. LCMS: m/z [M+H-tBu]⁺=244.2.

### Procedure for preparation of 3-fluoro-N, 2-dimethyl-4,5,6,7-tetrahydrobenzothiophen-5-amine hydrochloride (196)

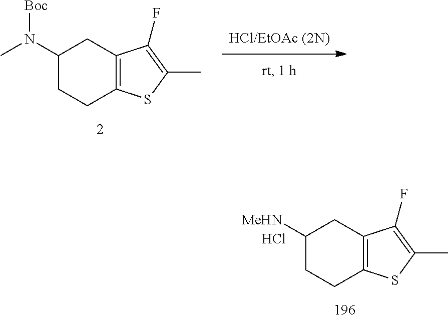

To a solution of tert-butyl N-(3-fluoro-2-methyl-4,5,6,7-tetrahydrobenzothiophen-5-yl)-N-methyl-carbamate (2) (280 mg, 935.19 μmol, 1 eq) in EtOAc (2 mL) was added HCl/EtOAc (4 M, 2 mL, 8.55 eq). The mixture was stirred at 25° C. for 1 hr. The reaction mixture was then filtered and the filter cake was washed with EtOAc (10 mL). Compound 3-fluoro-N, 2-dimethyl-4,5,6,7-tetrahydrobenzothiophen-5-amine hydrochloride (196) (214.66 mg, 908.54 μmol, 97.15% yield) was obtained as a white solid. ¹H NMR (400 MHz, METHANOL-d₄) δ=3.54-3.46 (m, 1H), 3.03 (dd, J=5.0, 16.3 Hz, 1H), 2.88-2.77 (m, 5H), 2.58-2.48 (m, 1H), 2.36-2.22 (m, 4H), 2.01-1.88 (m, 1H). LCMS: m/z [M+H]⁺=200.1.

### Synthetic scheme for preparation of N, 2-dimethyl-4,5,6,7-tetrahydrobenzothiophen-5-amine hydrochloride (197)

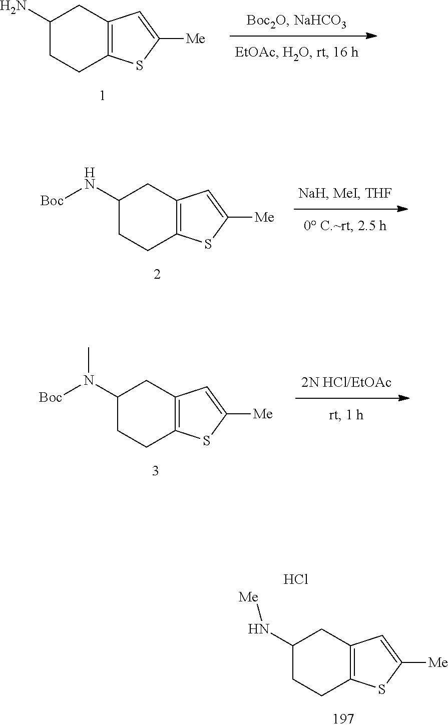 [SMILES: Cc1cc2c(s1)CCC(N)C2 ; Cc1cc2c(s1)CCC(NC(=O)OC(C)(C)C)C2 ; Cc1cc2c(s1)CCC(N(C)C(=O)OC(C)(C)C)C2 ; Cl ; CNC1CCc2sc(C)cc2C1]

### Procedure for preparation of tert-butyl N-(2-methyl-4,5,6,7-tetrahydrobenzothiophen-5-yl)carbamate (2)

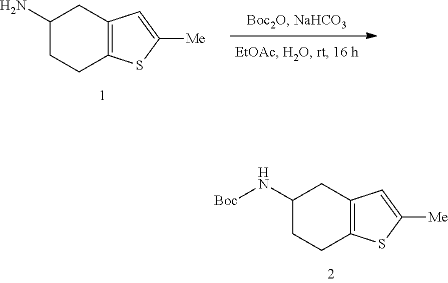 [SMILES: Cc1cc2c(s1)CCC(N)C2 ; Cc1cc2c(s1)CCC(NC(=O)OC(C)(C)C)C2]

To a solution of 2-methyl-4,5,6,7-tetrahydrobenzothiophen-5-amine (1) (120 mg, 717.40 μmol, 1 eq) in EtOAc (1 mL) and H₂O (1 mL) was added aq. NaHCO₃ (180.80 mg, 2.15 mmol, 83.74 μL, 3 eq) and (Boc)₂O (234.85 mg, 1.08 mmol, 247.22 μL, 1.5 eq). The mixture was stirred at 25° C. for 16 hr. The reaction mixture was diluted with water (10 mL) and then extracted with EtOAc 30 mL (10 mL*3). The combined organic layers were washed with brine (20 mL), dried over Na₂SO₄, filtered and concentrated under reduced pressure to give a residue. The residue was purified by flash silica gel chromatography (ISCO®; 12 g SepaFlash® Silica Flash Column, eluent 0~25% Ethyl acetate/Petroleum ether gradient @35 mL/min). Compound tert-butyl N-(2-methyl-4,5,6,7-tetrahydrobenzothiophen-5-yl)carbamate (2) (50 mg, 187.00 μmol, 26.07% yield) was obtained as a white solid. LCMS: m/z [M-tBu+H]⁺=211.8.

### Procedure for preparation of tert-butyl N-methyl-N-(2-methyl-4,5,6,7-tetrahydrobenzothiophen-5-yl)carbamate(3)

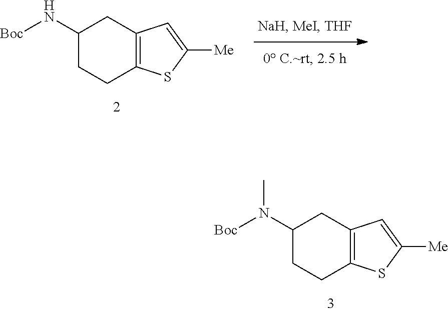 [SMILES: Cc1cc2c(s1)CCC(NC(=O)OC(C)(C)C)C2 ; Cc1cc2c(s1)CCC(N(C)C(=O)OC(C)(C)C)C2]

To a solution of tert-butyl N-(2-methyl-4,5,6,7-tetrahydrobenzothiophen-5-yl)carbamate (2) (50 mg, 187.00 μmol, 1 eq) in DMF (1 mL) was added NaH (9.72 mg, 243.09 μmol, 60% suspension in mineral oil, 1.3 eq) at 0° C. under N₂. The mixture was stirred at 0° C. for 0.5 h. Then to the mixture was added MeI (79.63 mg, 560.99 μmol, 34.92 L, 3 eq) at 0° C. The mixture was stirred at 25° C. for 2 h and then quenched with H₂O (10 mL). The reaction mixture was extracted with EtOAc (10 mL*3). The combined organic layers were washed with brine (20 mL), dried over Na₂SO₄, filtered and concentrated under reduced pressure to give a residue. The residue was purified by prep-TLC (SiO₂, Petroleum ether:Ethyl acetate=3:1). Compound tert-butyl N-methyl-N-(2-methyl-4,5,6,7-tetrahydrobenzothiophen-5-yl)carbamate (3) (47 mg, 167.01 μmol, 89.31% yield) was obtained as a white solid. LCMS: m/z [M-t-Bu+H]⁺=225.8.

### Procedure for preparation of N, 2-dimethyl-4,5,6,7-tetrahydrobenzothiophen-5-amine hydrochloride

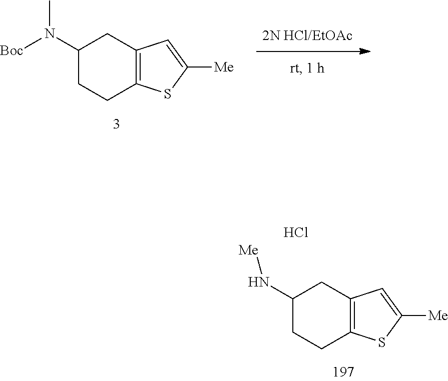 [SMILES: Cc1cc2c(s1)CCC(N(C)C(=O)OC(C)(C)C)C2 ; Cl ; CNC1CCc2sc(C)cc2C1]

To a solution of tert-butyl N-methyl-N-(2-methyl-4,5,6,7-tetrahydrobenzothiophen-5-yl)carbamate (3) (47 mg, 167.01 μmol, 1 eq) in EtOAc (1 mL) was added HCl/EtOAc (4 M, 1 mL, 23.95 eq). The mixture was stirred at 25° C. for 1 hr. The reaction mixture was concentrated under reduced pressure to give a residue. The residue was purified by prep-HPLC (column: Phenomenex luna C18 150*25 mm*10 μm; mobile phase: [water(HCl)-ACN]; gradient:1%-30% B over 10 min). Compound N, 2-dimethyl-4,5,6,7-tetrahydrobenzothiophen-5-amine hydrochloride (197) (7.53 mg, 34.58 μmol, 20.70% yield) was obtained as a white solid. LCMS: m/z [M+H]⁺=182.2. ¹H NMR (400 MHz, METHANOL-d4) δ 6.46 (s, 1H), 3.53-3.42 (m, 1H), 3.07 (dd, J=4.9, 15.7 Hz, 1H), 2.93-2.83 (m, 2H), 2.77 (s, 3H), 2.63 (br dd, J=9.1, 15.6 Hz, 1H), 2.38 (s, 3H), 2.34-2.26 (m, 1H), 1.94 (dddd, J=6.8, 9.0, 10.6, 12.7 Hz, 1H)

### Synthetic scheme for preparation of 3-chloro-N-methyl-4,5,6,7-tetrahydrobenzothiophen-6-amine hydrochloride enantiomer 1 (198) and enantiomer2 (199)

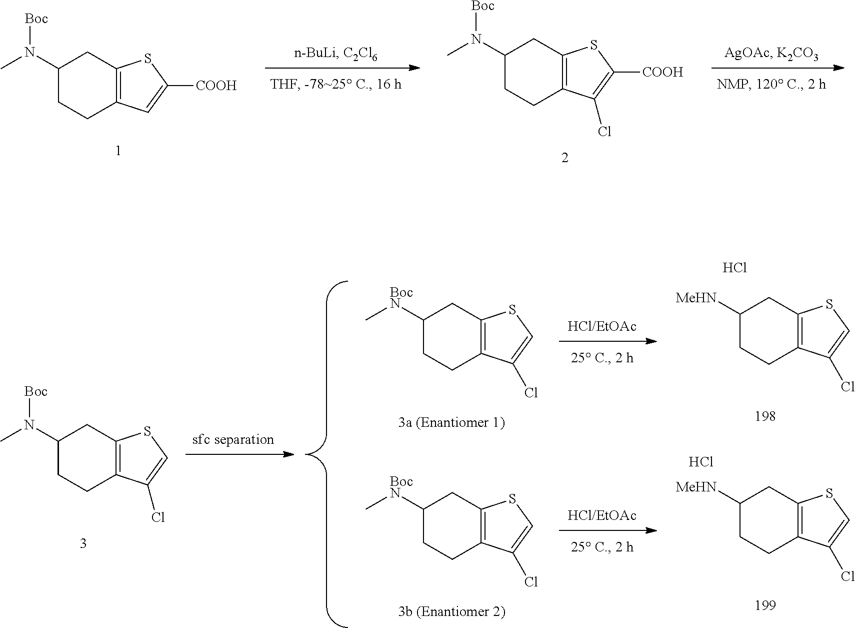

### Procedure for preparation of 6-[tert-butoxycarbonyl(methyl)amino]-3-chloro-4,5,6,7-tetrahydrobenzothiophene-2-carboxylic acid (2)

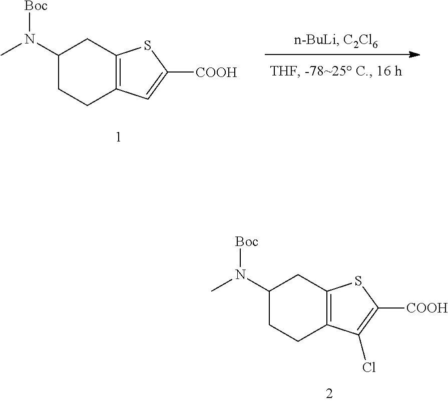 [SMILES: CN(C(=O)OC(C)(C)C)C1CCc2cc(C(=O)O)sc2C1 ; CN(C(=O)OC(C)(C)C)C1CCc2c(sc(C(=O)O)c2Cl)C1]

To a solution of 6-[tert-butoxycarbonyl(methyl)amino]-4,5,6,7-tetrahydrobenzothiophene-2-carboxylic acid (1) (1.5 g, 4.82 mmol, 1 eq) in THF (15 mL) was added n-BuLi (2.5 M, 3.85 mL, 2 eq) dropwise at −78° C. over 15 min. After addition, the mixture was stirred at this temperature for 15 min, and then 1,1,1,2,2,2-hexachloroethane (1.37 g, 5.78 mmol, 654.45 μL, 1.2 eq) in THF (5 mL) was added dropwise at −78° C. The resulting mixture was allowed to slowly warm up to 25° C. and stirred for 16 hr. The reaction mixture was poured into NH₄Cl aq. saturated solution (30 mL) and extracted with EtOAc (20 mL*2). The combined organic phases were washed with brine (10 mL), dried over anhydrous sodium sulfate, filtered and concentrated under vacuum to give a residue. The residue was purified by prep-HPLC (column: Phenomenex luna C18 (250*70 mm, 10 m); mobile phase: [water(FA)-ACN]; gradient:45%-75% B over 20 min) to afford 6-[tert-butoxycarbonyl(methyl)amino]-3-chloro-4,5,6,7-tetrahydrobenzothiophene-2-carboxylic acid (2) (500 mg, 1.45 mmol, 30.01% yield) as a yellow solid. ¹H NMR (400 MHz, CHLOROFORM-d) δ=4.63-4.16 (m, 1H), 3.01-2.72 (m, 6H), 2.69-2.51 (m, 1H), 2.09-1.79 (m, 2H), 1.49 (s, 9H)

### Procedure for preparation of tert-butyl N-(3-chloro-4,5,6,7-tetrahydrobenzothiophen-6-yl)-N-methyl-carbamate (3)

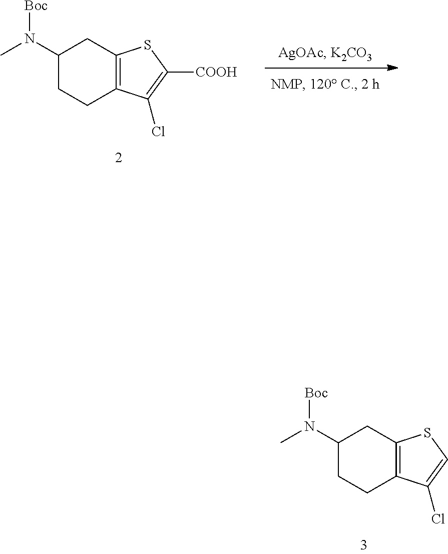 [SMILES: CN(C(=O)OC(C)(C)C)C1CCc2c(sc(C(=O)O)c2Cl)C1 ; CN(C(=O)OC(C)(C)C)C1CCc2c(Cl)csc2C1]

To a solution of 6-[tert-butoxycarbonyl(methyl)amino]-3-chloro-4,5,6,7-tetrahydrobenzothiophene-2-carboxylic acid (2) (500 mg, 1.45 mmol, 1 eq) in NMP (7 mL) was added AgOAc (48.26 mg, 289.15 μmol, 0.2 eq) and K₂CO₃ (599.45 mg, 4.34 mmol, 3 eq). The mixture was stirred at 120° C. for 2 hr then filtered and the filtrate was poured into H₂O (20 mL) and extracted with EtOAc (20 mL*3). The combined organic phases were washed with brine (10 mL), dried over anhydrous sodium sulfate, filtered and concentrated under vacuum to give a residue. The residue was purified by prep-HPLC (column: Phenomenex luna C18 150*40 mm*15 μm; mobile phase: [water(FA)-ACN]; gradient:65%-95% B over 15 min) to afford tert-butyl N-(3-chloro-4,5,6,7-tetrahydrobenzothiophen-6-yl)-N-methyl-carbamate (3) (400 mg, 1.33 mmol, 91.66% yield) as a brown solid. ¹H NMR (400 MHz, CHLOROFORM-d) δ=6.93 (s, 1H), 4.60-4.13 (m, 1H), 2.92-2.70 (m, 6H), 2.66-2.50 (m, 1H), 2.06-1.75 (m, 2H), 1.48 (s, 9H)

### Procedure for preparation of tert-butyl N-(3-chloro-4,5,6,7-tetrahydrobenzothiophen-6-yl)-N-methyl-carbamate enantiomer 1 (3a) and enantiomer 2 (3b)

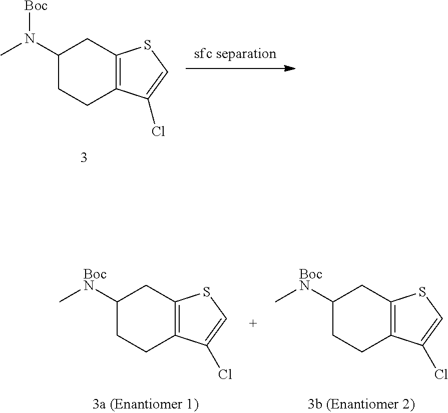 [SMILES: CN(C(=O)OC(C)(C)C)C1CCc2c(Cl)csc2C1 ; CN(C(=O)OC(C)(C)C)C1CCc2c(Cl)csc2C1 ; CN(C(=O)OC(C)(C)C)C1CCc2c(Cl)csc2C1]

Racemic tert-butyl N-(3-chloro-4,5,6,7-tetrahydrobenzothiophen-6-yl)-N-methyl-carbamate (3) was separated to individual enantiomers by SFC (column: DAICEL CHIRALPAK IG (250 mm*50 mm, 10 μm); mobile phase: [CO₂, i-PrOH (0.1% NH₃H₂O)]; B %:12%, isocratic elution mode). Enantiomer 2 (Peak2) was further purified by SFC (column: DAICEL CHIRALPAK IG (250 mm*30 mm, 10 μm); mobile phase: [CO₂, i-PrOH (0.1% NH₃H₂O)]; B %:10%, isocratic elution mode). Compound tert-butyl N-(3-chloro-4,5,6,7-tetrahydrobenzothiophen-6-yl)-N-methyl-carbamate enantiomer 1 (3a) (100 mg, 331.31 μmol, 45.45% yield) was obtained as yellow oil. Compound tert-butyl N-(3-chloro-4,5,6,7-tetrahydrobenzothiophen-6-yl)-N-methyl-carbamate enantiomer 2 (3b) (70 mg, 231.92 μmol, 31.82% yield) was obtained as yellow oil.

### Procedure for preparation of 3-chloro-N-methyl-4,5,6,7-tetrahydrobenzothiophen-6-amine hydrochloride enantiomer 1 (198)

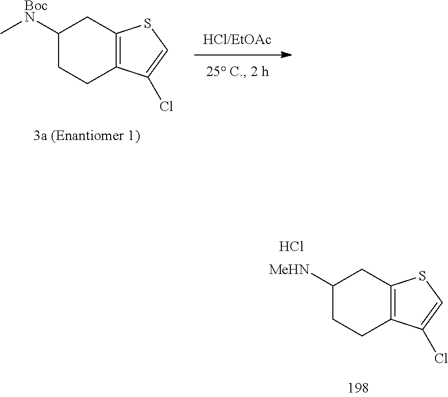 [SMILES: CN(C(=O)OC(C)(C)C)C1CCc2c(Cl)csc2C1 ; Cl ; CNC1CCc2c(Cl)csc2C1]

To a solution of tert-butyl N-(3-chloro-4,5,6,7-tetrahydrobenzothiophen-6-yl)-N-methyl-carbamate enantiomer 1 (3a) (100 mg, 331.31 μmol, 1 eq) in EtOAc (1 mL) was added HCl/EtOAc (4 M, 1 mL, 12.07 eq). The mixture was stirred at 25° C. for 2 hr. The mixture was concentrated and the residue was triturated with EtOAc at 25° C. for 10 min. The mixture was then filtered and, the filter cake was dried to afford 3-chloro-N-methyl-4,5,6,7-tetrahydrobenzothiophen-6-amine hydrochloride enantiomer 1 (198) (65.76 mg, 273.34 μmol, 82.50% yield) as a white solid. LCMS: m/z [M+H]⁺=202.1. ¹H NMR (400 MHz, METHANOL-d4) δ=7.21 (s, 1H), 3.63-3.50 (m, 1H), 2.97-2.83 (m, 1H), 2.82-2.70 (m, 4H), 2.70-2.60 (m, 1H), 2.36-2.25 (m, 1H), 2.02-1.89 (m, 1H)

### Procedure for preparation of 3-chloro-N-methyl-4,5,6,7-tetrahydrobenzothiophen-6-amine hydrochloride enantiomer 2 (199)

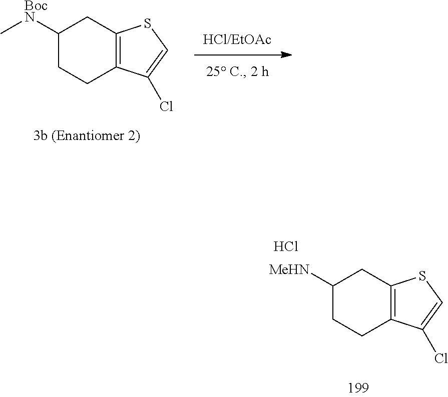 [SMILES: CN(C(=O)OC(C)(C)C)C1CCc2c(Cl)csc2C1 ; Cl ; CNC1CCc2c(Cl)csc2C1]

To a solution of tert-butyl N-(3-chloro-4,5,6,7-tetrahydrobenzothiophen-6-yl)-N-methyl-carbamate enantiomer 2 (3b) (70 mg, 231.92 μmol, 1 eq) in EtOAc (1 mL) was added HCl/EtOAc (4 M, 57.98 μL, 1 eq). The mixture was stirred at 25° C. for 2 hr. The mixture was concentrated and the residue was triturated with EtOAc at 25° C. for 10 min. The mixture was then filtered and the filter cake was dried to afford 3-chloro-N-methyl-4,5,6,7-tetrahydrobenzothiophen-6-amine hydrochloride enantiomer 2 (199) (54.69 mg, 222.73 μmol, 96.04% yield) as a white solid. LCMS: m/z [M+H]⁺=202.1. ¹H NMR (400 MHz, METHANOL-d4) δ=7.21 (s, 1H), 3.62-3.52 (m, 1H), 2.89 (br dd, J=8.1, 15.7 Hz, 1H), 2.83-2.72 (m, 4H), 2.71-2.60 (m, 1H), 2.37-2.26 (m, 1H), 2.02-1.90 (m, 1H)

### Synthetic scheme for preparation of 1,5-dimethyl-6,7-dihydro-4H-2-benzothiophen-5-amine hydrochloride (200)

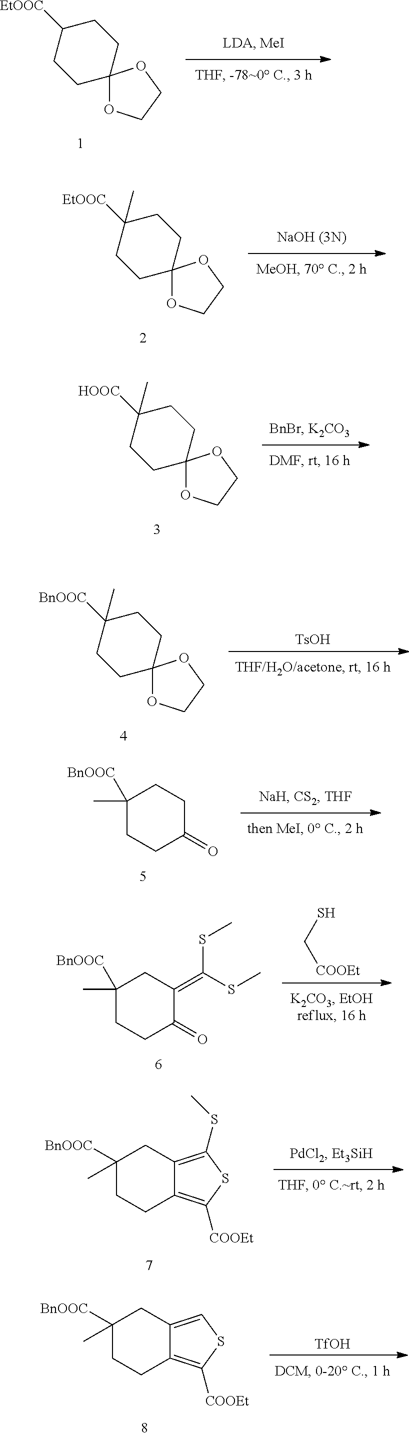

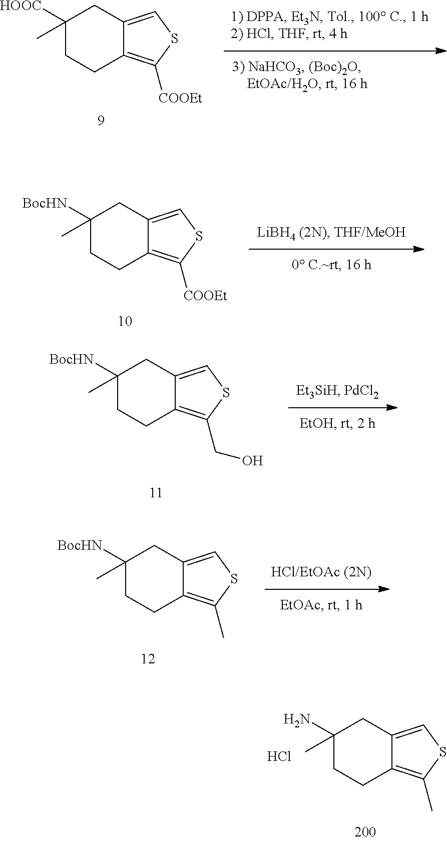 [SMILES: CCOC(=O)c1scc2c1CCC(C)(C(=O)O)C2 ; CCOC(=O)c1scc2c1CCC(C)(NC(=O)OC(C)(C)C)C2 ; CC1(NC(=O)OC(C)(C)C)CCc2c(csc2CO)C1 ; Cc1scc2c1CCC(C)(NC(=O)OC(C)(C)C)C2 ; Cl ; Cc1scc2c1CCC(C)(N)C2]

### Procedure for preparation of ethyl 8-methyl-1,4-dioxaspiro[4.5]decane-8-carboxylate (2)

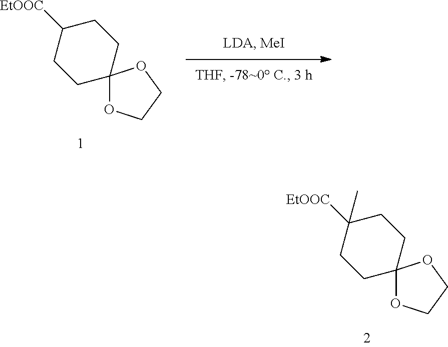 [SMILES: CCOC(=O)C1CCC2(CC1)OCCO2 ; CCOC(=O)C1(C)CCC2(CC1)OCCO2]

To a solution of LDA (2 M, 280.04 mL, 1.2 eq) in dry THF (600 mL) was added a solution of ethyl 1,4-dioxaspiro[4.5]decane-8-carboxylate (1) (100 g, 466.73 mmol, 1 eq) in dry THF (50 mL) dropwise at −78° C. under N₂. After 1 h CH₃I was added (86.12 g, 606.75 mmol, 37.77 mL, 1.3 eq) in dry THF (50 mL) dropwise. The mixture was stirred at −78° C. for 1 h, then was warmed to 0° C. for 1 h under N₂. The reaction mixture was quenched by addition aq. sat.NH₄Cl 400 mL at 0° C., and then extracted with EtOAc (600 mL*2). The combined organic layers were washed with brine (600 mL), dried over anhydrous Na₂SO₄, filtered and concentrated under reduced pressure to give ethyl 8-methyl-1,4-dioxaspiro[4.5]decane-8-carboxylate (2) (108 g, crude) as a brown oil. ¹H NMR (400 MHz, CHLOROFORM-d) δ=4.14 (q, J=7.1 Hz, 2H), 3.93 (s, 4H), 2.17-2.07 (m, 2H), 1.69-1.56 (m, 4H), 1.55-1.45 (m, 2H), 1.28-1.22 (t, 3H), 1.18 (s, 3H)

### Procedure for preparation of 8-methyl-1,4-dioxaspiro[4.5]decane-8-carboxylic acid (3)

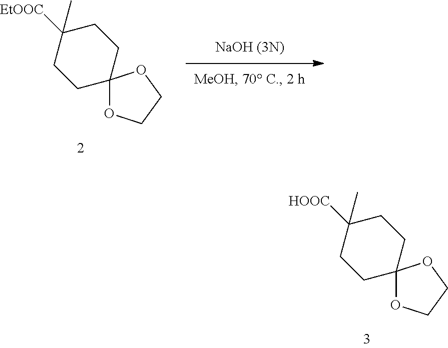 [SMILES: CCOC(=O)C1(C)CCC2(CC1)OCCO2 ; CC1(C(=O)O)CCC2(CC1)OCCO2]

To a solution of ethyl 8-methyl-1,4-dioxaspiro[4.5]decane-8-carboxylate (2) (30 g, 131.42 mmol, 1 eq) in MeOH (300 mL) was added NaOH (3 M in H₂O, 131.42 mL, 3 eq). The mixture was stirred at 70° C. for 2 hr. The reaction mixture was concentrated under reduced pressure to remove MeOH. The residue was diluted with H₂O 100 mL, pH adjusted to 4 with 1N aq. HCl at 0° C. The mixture was extracted with EtOAc (200 mL*2). The combined organic layers were washed with brine (200 mL), dried over anhydrous Na₂SO₄, filtered and concentrated under reduced pressure to give 8-methyl-1,4-dioxaspiro[4.5]decane-8-carboxylic acid (3) (25 g, crude) as a brown oil. ¹H NMR (400 MHz, CHLOROFORM-d) δ=3.95 (s, 4H), 2.19-2.09 (m, 2H), 1.72-1.64 (m, 4H), 1.61-1.49 (m, 2H), 1.26 (s, 3H).

### Procedure for preparation of benzyl 8-methyl-1,4-dioxaspiro[4.5]decane-8-carboxylate (4)

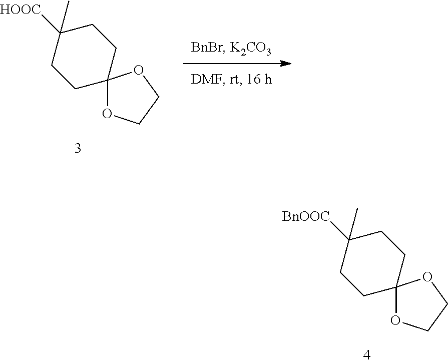

To a solution of 8-methyl-1,4-dioxaspiro[4.5]decane-8-carboxylic acid (3) (25 g, 124.86 mmol, 1 eq) in DMF (250 mL) was added K₂CO₃ (20.71 g, 149.83 mmol, 1.2 eq) and bromomethylbenzene (23.5 g, 137.34 mmol, 16.3 mL, 1.1 eq). The mixture was stirred at 20° C. for 16 hrs. The reaction mixture was filtered and diluted with H₂O 1 L, extracted with EtOAc (400 mL*2). The combined organic layers were washed with brine (400 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~15% Ethyl acetate/Petroleum ether gradient @45 mL/min) to give benzyl 8-methyl-1,4-dioxaspiro[4.5]decane-8-carboxylate (4) (34 g, 117.10 mmol, 93.79% yield) as a yellow oil. ¹H NMR (400 MHz, CHLOROFORM-d) δ=7.42-7.29 (m, 5H), 5.14 (s, 2H), 3.93 (s, 4H), 2.23-2.13 (m, 2H), 1.74-1.48 (m, 6H), 1.23 (s, 3H). LCMS: m/z [M+H]⁺=291.1

### Procedure for preparation of benzyl 1-methyl-4-oxo-cyclohexanecarboxylate (5)

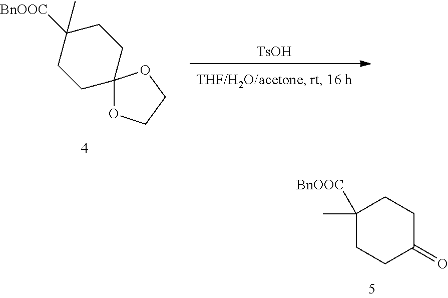

To a solution of benzyl 8-methyl-1,4-dioxaspiro[4.5]decane-8-carboxylate (4) (32 g, 110.21 mmol, 1 eq) in THF (300 mL), acetone (100 mL) and H₂O (400 mL) was added TsOH (37.96 g, 220.42 mmol, 2 eq). The mixture was stirred at 20° C. for 16 hrs. To the reaction mixture was added solid NaHCO₃ to adjust the pH to7, and the mixture was extracted with EtOAc (300 mL*2). The combined organic layers were washed with brine (300 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~10% Ethyl acetate/Petroleum ether gradient @50 mL/min) to give benzyl 1-methyl-4-oxo-cyclohexanecarboxylate (5) (27 g, 109.62 mmol, 99.47% yield) as a yellow oil. ¹H NMR (400 MHz, CHLOROFORM-d) δ=7.40-7.31 (m, 5H), 5.20 (s, 2H), 2.49-2.26 (m, 6H), 1.76-1.65 (m, 2H), 1.33 (s, 3H). LCMS: m/z [M+H]⁺=247.3

### Procedure for preparation of benzyl 3-[bis(methylsulfanyl)methylene]-1-methyl-4-oxo-cyclohexanecarboxylate (6)

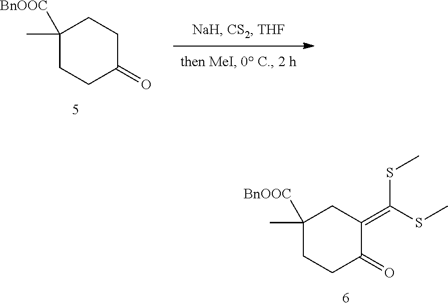

To a solution of benzyl 1-methyl-4-oxo-cyclohexanecarboxylate (5) (27 g, 109.62 mmol, 1 eq) in dry THF (300 mL) was added NaH (5.70 g, 142.51 mmol, 60% suspension in mineral oil, 1.3 eq) slowly at 0° C. The mixture was stirred for 0.5 h, then to the mixture added CS₂ (8.35 g, 109.62 mmol, 6.61 mL, 1 eq) dropwise at 0° C. After 0.5 h, MeI (34.2 g, 241.2 mmol, 15.0 mL, 2.2 eq) was then added to the mixture. The mixture was stirred at 0° C. for 1 h and then quenched with aq. sat.NH₄Cl 400 mL at 0° C., and extracted with EtOAc (300 mL*2). The combined organic layers were washed with brine (200 mL), dried over anhydrous Na₂SO₄, filtered and concentrated under reduced pressure to give a residue. The residue was purified by flash silica gel chromatography (ISCO®; 180 g SepaFlash® Silica Flash Column, eluent 0~25% Ethyl acetate/Petroleum ether gradient @80 mL/min) to give benzyl 3-[bis(methylsulfanyl)methylene]-1-methyl-4-oxo-cyclohexanecarboxylate (6) (29 g, 41.37 mmol, 37.7% yield) as a yellow oil. LCMS: m/z [M+H]⁺=350.9.

### Procedure for preparation of 5-benzyl 1-ethyl 5-methyl-3-(methylthio)-4,5,6,7-tetrahydrobenzo[c]thiophene-1,5-dicarboxylate (7)

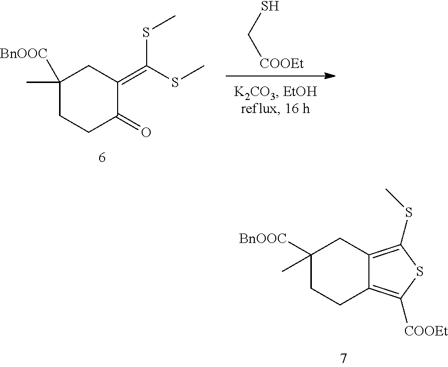

To a solution of benzyl 3-[bis(methylsulfanyl)methylene]-1-methyl-4-oxo-cyclohexanecarboxylate (6) (29 g, 82.74 mmol, 1 eq) in EtOH (300 mL) was added K₂CO₃ (17.15 g, 124.11 mmol, 1.5 eq) and ethyl 2-sulfanylacetate (11.24 g, 93.53 mmol, 10.26 mL, 1.13 eq). The mixture was stirred at 80° C. for 16 hrs. The reaction mixture was concentrated under reduced pressure to remove EtOH. The residue was concentrated under reduced pressure to give a residue. The residue was purified by flash silica gel chromatography (ISCO®; 180 g SepaFlash® Silica Flash Column, eluent 0~15% Ethyl acetate/Petroleum ether gradient @80 mL/min) to give 5-benzyl 1-ethyl 5-methyl-3-(methylthio)-4,5,6,7-tetrahydrobenzo[c]thiophene-1,5-dicarboxylate (7) (19 g, 28.18 mmol, 34.06% yield) as a yellow oil. LCMS: m/z [M+H]⁺=404.9

### Procedure for preparation of 5-benzyl 1-ethyl 5-methyl-4,5,6,7-tetrahydrobenzo[c]thiophene-1,5-dicarboxylate (8)

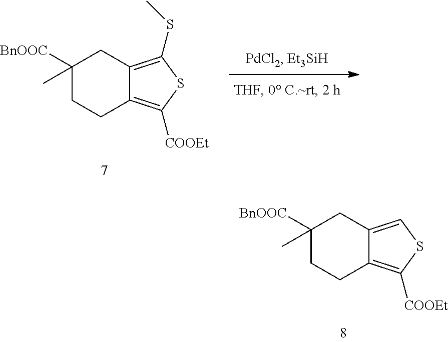

To a solution of 5-benzyl 1-ethyl 5-methyl-3-(methylthio)-4,5,6,7-tetrahydrobenzo[c]thiophene-1,5-dicarboxylate (7) (10 g, 14.83 mmol, 1 eq, 60% purity) in THF (150 mL) was added PdCl₂ (263.00 mg, 1.48 mmol, 0.1 eq) and Et₃SiH (3.45 g, 29.66 mmol, 4.74 mL, 2 eq) at 0° C. under N₂. The mixture was stirred at 20° C. for 2 hrs. The reaction mixture was filtered and concentrated under reduced pressure to give a residue. The residue was purified by flash silica gel chromatography (ISCO®; 80 g SepaFlash® Silica Flash Column, eluent 0~15% Ethyl acetate/Petroleum ether gradient @60 mL/min) to give 5-benzyl 1-ethyl 5-methyl-4,5,6,7-tetrahydrobenzo[c]thiophene-1,5-dicarboxylate (8) (6.3 g, 12.48 mmol, 84.14% yield) as a yellow oil. LCMS: m/z [M+H]⁺=358.9

### Procedure for preparation of 1-ethoxycarbonyl-5-methyl-6,7-dihydro-4H-2-benzothiophene-5-carboxylic acid (9)

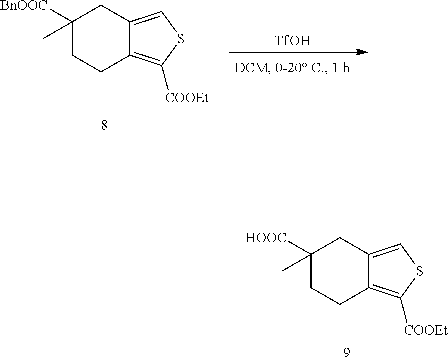

To a solution of 5-benzyl 1-ethyl 5-methyl-4,5,6,7-tetrahydrobenzo[c]thiophene-1,5-dicarboxylate (8) (6 g, 11.88 mmol, 1 eq) in DCM (80 mL) was added TfOH (8.92 g, 59.42 mmol, 5.26 mL, 5 eq) at 0° C. The mixture was stirred at 20° C. for 1 hr. The reaction mixture was concentrated under reduced pressure to remove DCM. The reaction mixture was diluted with H₂O (50 mL) and extracted with EtOAc (50 mL*2). The combined organic layers were washed with brine (50 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~25% Ethyl acetate/Petroleum ether gradient @40 mL/min) to give 1-ethoxycarbonyl-5-methyl-6,7-dihydro-4H-2-benzothiophene-5-carboxylic acid (9) (2.6 g, 9.69 mmol, 81.53% yield) as a yellow oil. ¹H NMR (400 MHz, CHLOROFORM-d) δ=7.12 (s, 1H), 4.37-4.30 (m, 2H), 3.22-3.11 (m, 3H), 2.60 (d, J=16.3 Hz, 1H), 2.23-2.15 (m, 1H), 1.86-1.78 (m, 1H), 1.37 (t, J=7.1 Hz, 3H), 1.32 (s, 3H). LCMS: m/z [M+H]⁺=268.8

### Procedure for preparation of ethyl 5-(tert-butoxycarbonylamino)-5-methyl-6,7-dihydro-4H-2-benzothiophene-1-carboxylate (10)

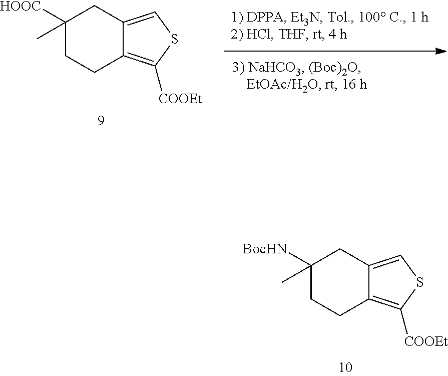 [SMILES: CCOC(=O)c1scc2c1CCC(C)(C(=O)O)C2 ; CCOC(=O)c1scc2c1CCC(C)(NC(=O)OC(C)(C)C)C2]

To a solution of 1-ethoxycarbonyl-5-methyl-6,7-dihydro-4H-2-benzothiophene-5-carboxylic acid (9) (2.6 g, 9.69 mmol, 1 eq) in Tol. (40 mL) was added DPPA (4.00 g, 14.53 mmol, 3.14 mL, 1.5 eq) and Et₃N (1.96 g, 19.38 mmol, 2.70 mL, 2 eq). The mixture was stirred at 100° C. for 1 h. To the reaction mixture it was then added aq. sat. NaHCO₃ (40 mL) and the mixture extracted with EtOAc (30 mL*2). The combined organic layers were washed with brine (30 mL), dried over anhydrous Na₂SO₄, filtered and concentrated under reduced pressure to give a residue. The residue was dissolved in THF (20 mL) and to the mixture it was added HCl (aq. 12 M, 2.42 mL, 3 eq) at 0° C. and the reaction was stirred at 20° C. for 4 h. To the reaction mixture was added NaHCO₃ (s) to adjust pH to 7, then was added EtOAc (20 mL), H₂O (20 mL), NaHCO₃ (2.44 g, 29.07 mmol 3 eq), Boc₂O (4.23 g, 19.38 mmol, 4.45 mL, 2 eq), and this mixture was stirred at 20° C. for 16 hrs. The reaction mixture was then diluted with H₂O 100 mL and extracted with EtOAc (50 mL*3). The combined organic layers were washed with brine (50 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0-10% Ethyl acetate/Petroleum ether gradient @40 mL/min) and by prep-HPLC (column: Kromasil Eternity XT 250*80 mm*10 μm; mobile phase: [water(NH4HCO3)-ACN]; gradient:55%-85% B over 20 min) to give ethyl 5-(tert-butoxycarbonylamino)-5-methyl-6,7-dihydro-4H-2-benzothiophene-1-carboxylate (10) (900 mg, 2.65 mmol, 27.36% yield) as a white solid. ¹H NMR (400 MHz, CHLOROFORM-d) δ=7.10 (s, 1H), 4.46-4.37 (m, 1H), 4.33 (q, J=7.1 Hz, 2H), 3.17 (td, J=5.7, 18.9 Hz, 1H), 3.01-2.89 (m, 2H), 2.75 (br d, J=16.3 Hz, 1H), 2.42-2.32 (m, 1H), 1.67 (ddd, J=6.3, 9.3, 13.6 Hz, 1H), 1.50-1.33 (m, 15H)

### Procedure for preparation of tert-butyl N-[1-(hydroxymethyl)-5-methyl-6,7-dihydro-4H-2-benzothiophen-5-yl]carbamate (11)

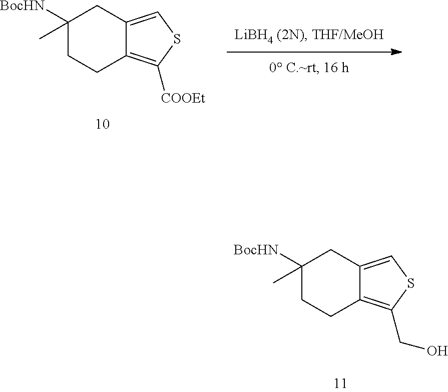 [SMILES: CCOC(=O)c1scc2c1CCC(C)(NC(=O)OC(C)(C)C)C2 ; CC1(NC(=O)OC(C)(C)C)CCc2c(csc2CO)C1]

To a solution of ethyl 5-(tert-butoxycarbonylamino)-5-methyl-6,7-dihydro-4H-2-benzothiophene-1-carboxylate (10) (400 mg, 1.18 mmol, 1 eq) in THF (5 mL) it was added LiBH₄ (2 M, 1.8 mL, 3.06 eq) and MeOH (0.5 mL) at 0° C. The mixture was stirred at 20° C. for 16 hrs. The reaction mixture was quenched by addition aq. sat.NH₄Cl (20 mL) at 0° C., and extracted with EtOAc (20 mL*3). The combined organic layers were washed with brine (20 mL), dried over anhydrous Na₂SO₄, filtered and concentrated under reduced pressure to give tert-butyl N-[1-(hydroxymethyl)-5-methyl-6,7-dihydro-4H-2-benzothiophen-5-yl]carbamate (11) (350 mg, crude) as a white solid. ¹H NMR (400 MHz, CHLOROFORM-d) δ=6.85 (s, 1H), 4.79-4.70 (m, 2H), 4.41 (br s, 1H), 2.88-2.79 (m, 1H), 2.79-2.68 (m, 2H), 2.66-2.55 (m, 1H), 2.45 (br d, J=12.5 Hz, 1H), 1.74-1.65 (m, 1H), 1.43 (br d, J=5.9 Hz, 12H). LCMS: m/z [M-tBu-OH]⁺=223.8.

### Procedure for preparation of tert-butyl N-(1,5-dimethyl-6,7-dihydro-4H-2-benzothiophen-5-yl)carbamate (12)

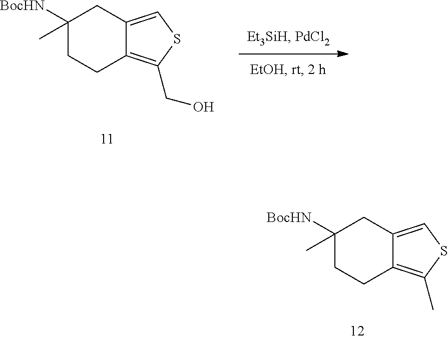 [SMILES: CC1(NC(=O)OC(C)(C)C)CCc2c(csc2CO)C1 ; Cc1scc2c1CCC(C)(NC(=O)OC(C)(C)C)C2]

The mixture was stirred at 20° C. for 2 hr. The reaction mixture was filtered, the pH of the filtrate was adjusted to 7 with NH₃·H₂O, then the resulting mixture was concentrated under reduced pressure to give a residue. The residue was purified by flash silica gel chromatography (ISCO®; 12 g SepaFlash® Silica Flash Column, eluent 0~10% Ethyl acetate/Petroleum ether gradient @30 mL/min) to give tert-butyl N-(1,5-dimethyl-6,7-dihydro-4H-2-benzothiophen-5-yl)carbamate (12) (200 mg, 710.70 μmol, 64.05% yield) as a white solid. ¹H NMR (400 MHz, CHLOROFORM-d) δ=6.65 (s, 1H), 4.41 (br s, 1H), 2.83-2.67 (m, 2H), 2.65-2.55 (m, 1H), 2.53-2.40 (m, 2H), 2.32 (s, 3H), 1.71-1.60 (m, 1H), 1.44 (d, J=2.6 Hz, 12H). LCMS: m/z [M-tBu+H]⁺=225.8.

### Procedure for preparation of 1,5-dimethyl-6,7-dihydro-4H-2-benzothiophen-5-amine (200)

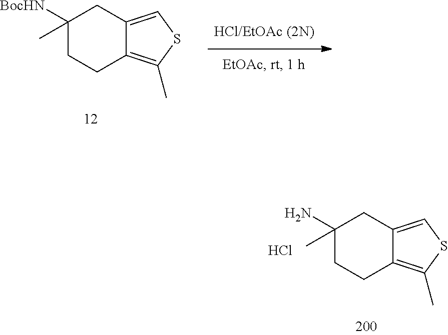 [SMILES: Cc1scc2c1CCC(C)(NC(=O)OC(C)(C)C)C2 ; Cl ; Cc1scc2c1CCC(C)(N)C2]

To a solution of tert-butyl N-(1,5-dimethyl-6,7-dihydro-4H-2-benzothiophen-5-yl)carbamate (12) (85 mg, 302.05 μmol, 1 eq) in EtOAc (1 mL) was added HCl/EtOAc (1 mL, 4N). The mixture was stirred at 20° C. for 1 hr. The reaction mixture was concentrated under reduced pressure to give a residue. The crude product was triturated with EtOAc (2 mL) at 20° C. for 5 min, filtered and dried under reduced pressure to give 1,5-dimethyl-6,7-dihydro-4H-2-benzothiophen-5-amine hydrochloride (200) (63.61 mg, 289.19 μmol, 95.74% yield) as a white solid. ¹H NMR (400 MHz, METHANOL-d4) δ=6.84 (s, 1H), 2.95-2.83 (m, 2H), 2.79-2.64 (m, 2H), 2.33 (s, 3H), 2.08-1.94 (m, 2H), 1.37 (s, 3H). LCMS: m/z [M+H]⁺=181.

### Synthetic scheme for preparation of 3-cyclopropyl-N-methyl-4,5,6,7-tetrahydrobenzothiophen-5-amine hydrochloride (201)

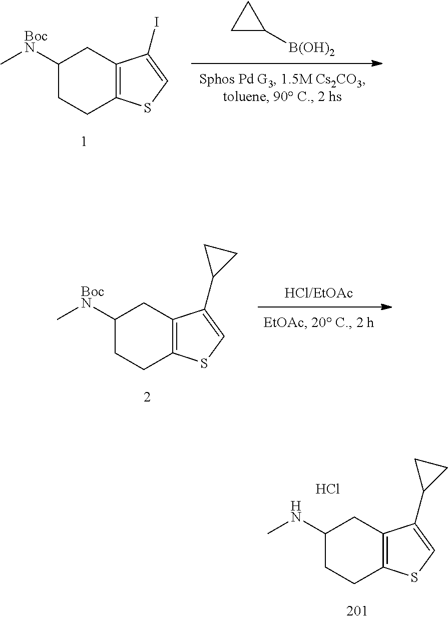 [SMILES: CN(C(=O)OC(C)(C)C)C1CCc2scc(I)c2C1 ; OB(O)C1CC1 ; CN(C(=O)OC(C)(C)C)C1CCc2scc(C3CC3)c2C1 ; CNC1CCc2scc(C3CC3)c2C1 ; Cl]

### Procedure for preparation of tert-butyl N-(3-iodo-4,5,6,7-tetrahydrobenzothiophen-5-yl)-N-methyl-carbamate (1)

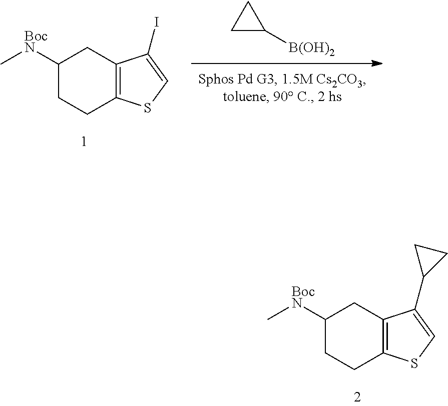 [SMILES: CN(C(=O)OC(C)(C)C)C1CCc2scc(I)c2C1 ; OB(O)C1CC1 ; CN(C(=O)OC(C)(C)C)C1CCc2scc(C3CC3)c2C1]

To a solution of tert-butyl N-(3-iodo-4,5,6,7-tetrahydrobenzothiophen-5-yl)-N-methyl-carbamate (1) (90 mg, 228.84 μmol, 1 eq) in toluene (1.5 mL) was added cyclopropylboronic acid (58.97 mg, 686.53 μmol, 3 eq), SPhos Pd G3 (17.86 mg, 22.88 μmol, 0.1 eq) and Cs₂CO₃ (1.5 M, 457.69 μL, 3 eq). The mixture was stirred at 90° C. for 2 hr. The mixture was then concentrated under reduced pressure to remove solvent. The residue was purified with prep-TLC (SiO₂, Petroleum ether/Ethyl acetate=5:1) and then withprep-HPLC (column: Phenomenex luna C18 150*25 mm*10 μm; mobile phase: [water (HCl)-ACN]; gradient:58%-88% B over 10 min). Compound tert-butyl N-(3-cyclopropyl-4,5,6,7-tetrahydrobenzothiophen-5-yl)-N-methyl-carbamate (2) (40 mg, 130.10 μmol, 56.85% yield) was obtained as a colorless oil.

### Procedure for preparation of 3-cyclopropyl-N-methyl-4,5,6,7-tetrahydrobenzothiophen-5-amine hydrochloride (201)

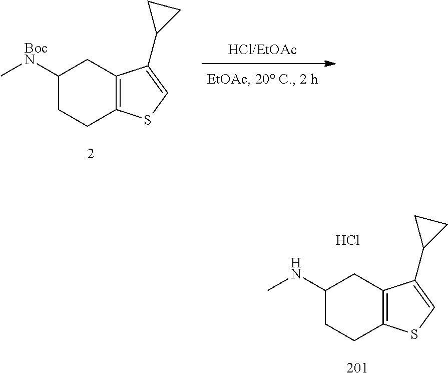 [SMILES: CN(C(=O)OC(C)(C)C)C1CCc2scc(C3CC3)c2C1 ; CNC1CCc2scc(C3CC3)c2C1 ; Cl]

To a solution of tert-butyl N-(3-cyclopropyl-4,5,6,7-tetrahydrobenzothiophen-5-yl)-N-methyl-carbamate (2) (40 mg, 130.10 μmol, 1 eq) in EtOAc (1 mL) was added HCl/EtOAc (4 M, 1 mL). The mixture was stirred at 20° C. for 2 hr and then concentrated under vacuum. Compound 3-cyclopropyl-N-methyl-4,5,6,7-tetrahydrobenzothiophen-5-amine hydrochloride (201) (25.44 mg, 117.53 μmol, 90.34% yield) was obtained as a white solid. ¹H NMR (400 MHz, METHANOL-d₄) δ=6.68 (s, 1H), 3.54 (br s, 1H), 3.23 (br dd, J=4.9, 15.8 Hz, 1H), 3.04-2.87 (m, 2H), 2.82 (s, 3H), 2.72 (br dd, J=9.1, 15.6 Hz, 1H), 2.44-2.28 (m, 1H), 2.08-1.90 (m, 1H), 1.76-1.64 (m, 1H), 0.96-0.77 (m, 2H), 0.55 (br dd, J=2.0, 4.9 Hz, 2H). LC-MS: m/z [M+H]⁺=208.1.

### Synthetic scheme for preparation of N, 2,3-trimethyl-6,7-dihydro-5H-thieno[3,2-b]pyran-6-amine hydrochloride (202)

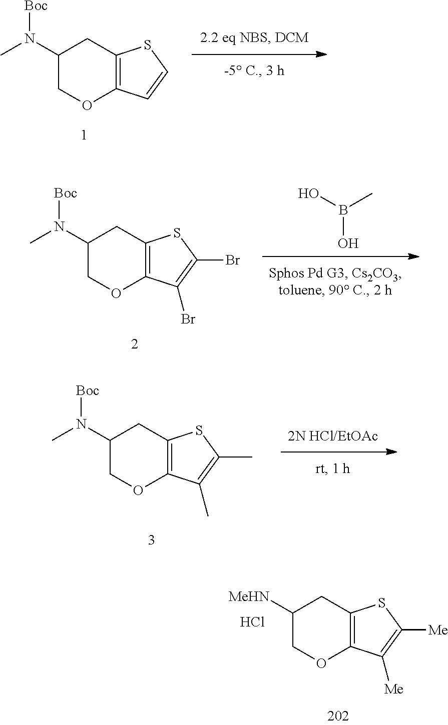

### Procedure for preparation of tert-butyl N-(2,3-dibromo-6,7-dihydro-5H-thieno[3,2-b]pyran-6-yl)-N-methyl-carbamate (2)

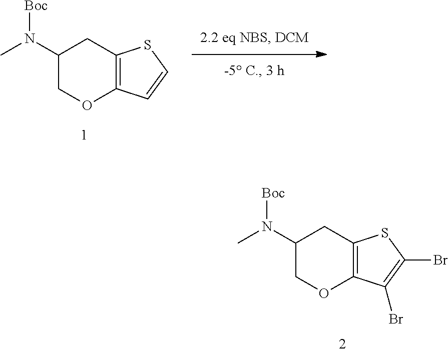 [SMILES: CN(C(=O)OC(C)(C)C)C1COc2ccsc2C1 ; CN(C(=O)OC(C)(C)C)C1COc2c(sc(Br)c2Br)C1]

To a solution of tert-butyl N-(6,7-dihydro-5H-thieno[3,2-b]pyran-6-yl)-N-methyl-carbamate (1) (2 g, 7.43 mmol, 1 eq) in DCM (20 mL) and AcOH (5 mL) was added NBS (2.91 g, 16.34 mmol, 2.2 eq). The mixture was stirred at −5° C. for 3 hr. The reaction mixture was quenched by addition aq. sat. Na₂SO₃ (100 mL) and aq sat. NaHCO₃ (40 ml) and extracted with DCM (10 mL*2). The combined organic layers were washed with aq. sat. NaCl (10 mL*2), dried over Na₂SO₄, filtered and concentrated under reduced pressure to give a residue. The residue was purified by prep-HPLC (column: Phenomenex luna C18 150*40 mm*15 μm; mobile phase: [water(FA)-ACN]; gradient:60%-90% B over 15 min). Compound tert-butyl N-(2,3-dibromo-6,7-dihydro-5H-thieno[3,2-b]pyran-6-yl)-N-methyl-carbamate (2) (500 mg, 1.17 mmol, 15.76% yield) was obtained as a white solid. LCMS: m/z [M+H-tBu]⁺=369.1

### Procedure for preparation of tert-butyl N-(2,3-dimethyl-6,7-dihydro-5H-thieno[3,2-b]pyran-6-yl)-N-methyl-carbamate (3)

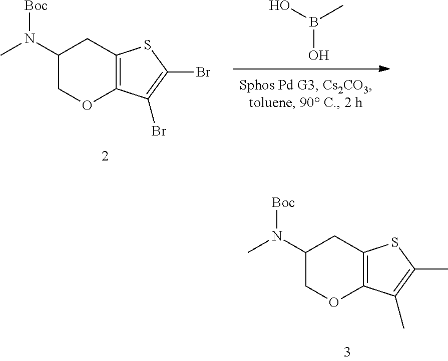

To a solution of tert-butyl N-(2,3-dibromo-6,7-dihydro-5H-thieno[3,2-b]pyran-6-yl)-N-methyl-carbamate (2) (230 mg, 538.45 μmol, 1 eq) in toluene (10 mL) was added methylboronic acid (322.32 mg, 5.38 mmol, 10 eq), Sphos Pd G3 (84.03 mg, 107.69 μmol, 0.2 eq) and cesium carbonate (1.05 g, 3.23 mmol, 6 eq) and purged with N₂ for 3 times. The mixture was stirred at 90° C. for 2 hr under N₂ atmosphere. The reaction mixture was concentrated under reduced pressure to remove toluene. The residue was purified by prep-HPLC (neutral condition:column: Waters Xbridge 150*25 mm*5 μm; mobile phase: [water(NH₄HCO₃)-ACN]; gradient:62%-92% B over 9 min). Compound tert-butyl N-(2,3-dimethyl-6,7-dihydro-5H-thieno[3,2-b]pyran-6-yl)-N-methyl-carbamate (3) (60 mg, 201.74 μmol, 37.47% yield) was obtained as a white solid. LCMS: m/z [M+H-tBu]⁺=242

### Procedure for preparation of N, 2,3-trimethyl-6,7-dihydro-5H-thieno[3,2-b]pyran-6-amine hydrochloride (202)

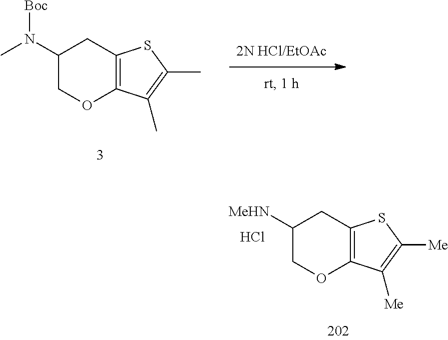

To a solution of tert-butyl N-(2,3-dimethyl-6,7-dihydro-5H-thieno[3,2-b]pyran-6-yl)-N-methyl-carbamate (3) (60 mg, 201.74 μmol, 1 eq) in EtOAc (2 mL) was added HCl/EtOAc (4 M, 2 mL). The mixture was stirred at 25° C. for 1 hr. The reaction mixture was concentrated under reduced pressure to remove EtOAc. The residue was purified by prep-HPLC (column: Phenomenex luna C18 150*25 mm*10 μm; mobile phase: [water(HCl)-ACN]; gradient:1%-30% B over 10 min). Compound N, 2,3-trimethyl-6,7-dihydro-5H-thieno[3,2-b]pyran-6-amine hydrochloride (202) (8.57 mg, 36.43 μmol, 18.06% yield) as a yellow solid. LCMS: m/z [M+H]⁺=198.1. ¹H NMR (400 MHz, METHANOL-d₄) δ=4.56-4.42 (m, 1H), 4.18 (d, J=11.9 Hz, 1H), 3.82-3.72 (m, 1H), 3.24 (br dd, J=5.6, 17.4 Hz, 1H), 2.89 (br d, J=17.8 Hz, 1H), 2.80 (s, 3H), 2.26 (s, 3H), 1.95 (s, 3H)

### Synthetic scheme for synthesis of 1-fluoro-3-methyl-4,5,6,7-tetrahydro-2-benzothiophen-5-amine hydrochloride (203)

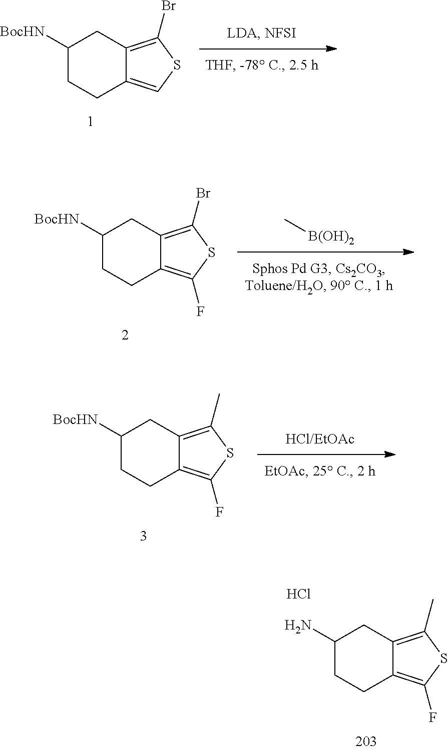

### Procedure for preparation of tert-butyl N-(3-bromo-1-fluoro-4,5,6,7-tetrahydro-2-benzothiophen-5-yl)carbamate (2)

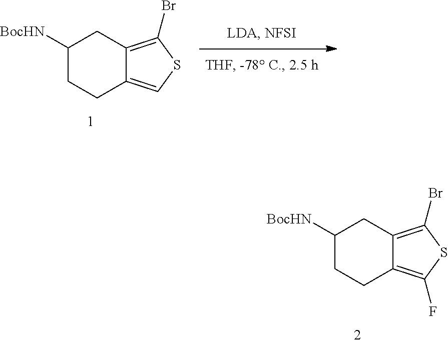 [SMILES: CC(C)(C)OC(=O)NC1CCc2csc(Br)c2C1 ; CC(C)(C)OC(=O)NC1CCc2c(F)sc(Br)c2C1]

To a solution of tert-butyl N-(3-bromo-4,5,6,7-tetrahydro-2-benzothiophen-5-yl)carbamate (1) (400 mg, 1.20 mmol, 1 eq) in THF (2 mL) was added LDA (2 M, 1.50 mL, 2.5 eq) at −78° C. The mixture was stirred at −78° C. for 0.5 h. Then to the mixture was added NFSI (383.43 mg, 1.22 mmol, 1.01 eq) at −78° C. under N₂. The mixture was stirred at −78° C. for 2 h. The mixture was quenched with saturated ammonium chloride aqueous solution (10 mL) and then the mixture was extracted with EtOAc (10 mL*3). The combined organic phases were washed with brine (10 mL), dried over Na₂SO₄, filtered and concentrated at reduced pressure to give a residue. The residue was purified by flash silica gel chromatography (ISCO®; 12 g SepaFlash® Silica Flash Column, eluent 0~30% Ethyl acetate/Petroleum ether gradient @25 mL/min). Compound tert-butyl N-(3-bromo-1-fluoro-4,5,6,7-tetrahydro-2-benzothiophen-5-yl)carbamate (2) (245 mg, 699.51 μmol, 58.10% yield) was obtained as a white solid. LCMS: m/z [M-tBu+H]⁺=295.5.

### Procedure for preparation of tert-butyl N-(1-fluoro-3-methyl-4,5,6,7-tetrahydro-2-benzothiophen-5-yl)carbamate (3)

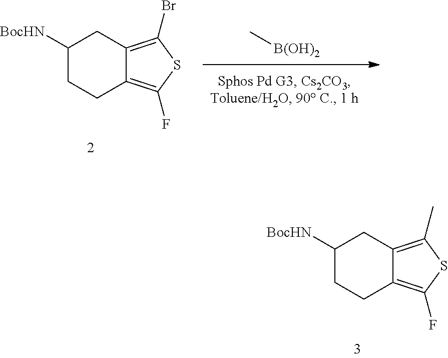

To a solution of tert-butyl N-(3-bromo-1-fluoro-4,5,6,7-tetrahydro-2-benzothiophen-5-yl)carbamate (2) (200 mg, 571.03 μmol, 1 eq), Cs₂CO₃ (372.10 mg, 1.14 mmol, 2 eq) and methylboronic acid (170.91 mg, 2.86 mmol, 5 eq) in toluene (1 mL) and H₂O (0.1 mL) was added SPhos Pd G3 (44.55 mg, 57.10 μmol, 0.1 eq). The mixture was stirred at 90° C. for 1 hr. The mixture was extracted with EtOAc (20 mL*3). The combined organic phases were washed with brine (10 mL), dried over Na₂SO₄, filtered and concentrated at reduced pressure to give a residue. The residue was purified by flash silica gel chromatography (ISCO®; 4 g SepaFlash® Silica Flash Column, eluent 0~30% Ethyl acetate/Petroleum ether gradient @25 mL/min). Compound tert-butyl N-(1-fluoro-3-methyl-4,5,6,7-tetrahydro-2-benzothiophen-5-yl)carbamate (3) (168 mg, 276.69 μmol, 48.45% yield) was obtained as a white solid. LCMS: m/z [M-tBu+H]⁺=230.0.

### Procedure for preparation of 1-fluoro-3-methyl-4,5,6,7-tetrahydro-2-benzothiophen-5-amine hydrochloride (203)

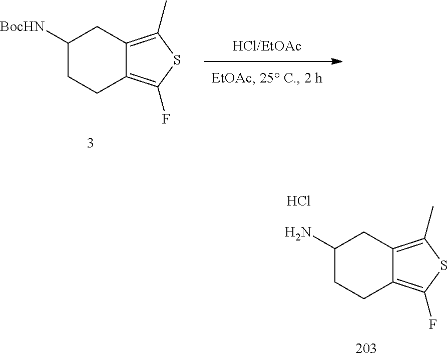 [SMILES: Cc1sc(F)c2c1CC(NC(=O)OC(C)(C)C)CC2 ; Cl ; Cc1sc(F)c2c1CC(N)CC2]

To a solution of tert-butyl N-(1-fluoro-3-methyl-4,5,6,7-tetrahydro-2-benzothiophen-5-yl)carbamate (3) (168 mg, 276.69 μmol, 1 eq) in EtOAc (1 mL) was added HCl/EtOAc (4 M, 1.00 mL, 14.46 eq). The mixture was stirred at 25° C. for 2 hr. The reaction mixture was concentrated under reduced pressure to give a residue. The residue was purified by prep-HPLC (column: Phenomenex luna C18 150*25 mm*10 μm; mobile phase: [water(HCl)-ACN]; gradient:1%-30% B over 10 min). Compound 1-fluoro-3-methyl-4,5,6,7-tetrahydro-2-benzothiophen-5-amine hydrochloride (203) (31.72 mg, 143.06 μmol, 51.71% yield) was obtained as a white solid. LCMS: m/z [M+H]⁺=186.1. ¹H NMR (400 MHz, METHANOL-d₄) δ=3.48 (ddt, J=2.9, 5.2, 10.5 Hz, 1H), 3.05 (dd, J=5.1, 15.8 Hz, 1H), 2.91-2.80 (m, 1H), 2.60 (dddd, J=2.6, 5.8, 11.1, 16.9 Hz, 1H), 2.47 (dd, J=10.1, 15.7 Hz, 1H), 2.23 (d, J=2.5 Hz, 3H), 2.19-2.09 (m, 1H), 1.76 (dtd, J=5.7, 11.1, 12.7 Hz, 1H)

### Synthetic scheme for preparation of 6-amino-4,5,6,7-tetrahydro-2-benzothiophene-1-carbonitrile (204)

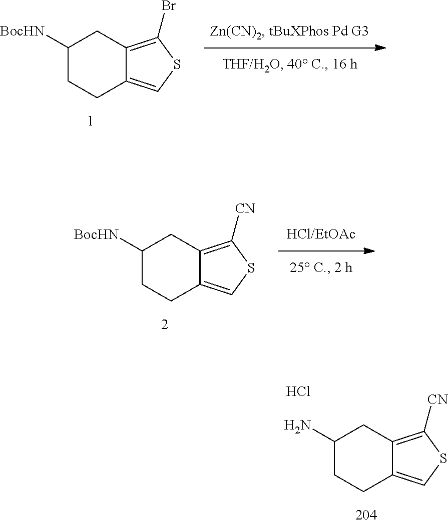 [SMILES: CC(C)(C)OC(=O)NC1CCc2csc(Br)c2C1 ; CC(C)(C)OC(=O)NC1CCc2csc(C#N)c2C1 ; Cl ; N#Cc1scc2c1CC(N)CC2]

### Procedure for preparation of tert-butyl N-(3-cyano-4,5,6,7-tetrahydro-2-benzothiophen-5-yl)carbamate (2)

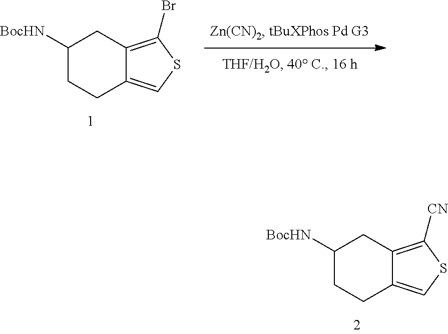 [SMILES: CC(C)(C)OC(=O)NC1CCc2csc(Br)c2C1 ; CC(C)(C)OC(=O)NC1CCc2csc(C#N)c2C1]

To a solution of tert-butyl N-(3-bromo-4,5,6,7-tetrahydro-2-benzothiophen-5-yl)carbamate (1) (100 mg, 300.97 μmol, 1 eq) in THF (1 mL) and H₂O (0.2 mL) was added Zn(CN)₂ (100 mg, 851.61 μmol, 2.83 eq) and tBuXPhos Pd G3 (23.91 mg, 30.10 μmol, 0.1 eq) under N₂. The mixture was stirred at 40° C. for 16 hrs. The reaction mixture was filtered and concentrated under reduced pressure to give a residue. The residue was purified by flash silica gel chromatography (ISCO®; 12 g SepaFlash® Silica Flash Column, eluent 0-30% Ethyl acetate/Petroleum ether gradient @35 mL/min). Compound tert-butyl N-(3-cyano-4,5,6,7-tetrahydro-2-benzothiophen-5-yl)carbamate (2) (38 mg, 136.51 μmol, 45.36% yield) was obtained as a white solid. LCMS: m/z [M-tBu+H]⁺=222.8.

### Procedure for preparation of 6-amino-4,5,6,7-tetrahydro-2-benzothiophene-1-carbonitrile hydrochloride (204)

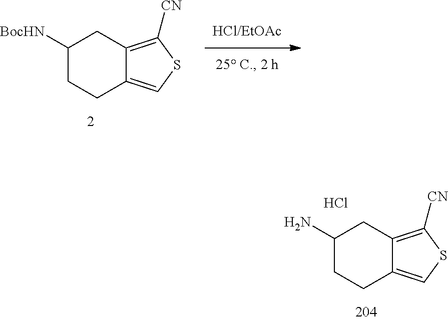 [SMILES: CC(C)(C)OC(=O)NC1CCc2csc(C#N)c2C1 ; N#Cc1scc2c1CC(N)CC2 ; Cl]

To a solution of tert-butyl N-(3-cyano-4,5,6,7-tetrahydro-2-benzothiophen-5-yl)carbamate (2) (38 mg, 136.51 μmol, 1 eq) in EtOAc (1 mL) was added HCl/EtOAc (4 M, 1 mL). The mixture was stirred at 25° C. for 2 hr. Then the reaction mixture was concentrated under reduced pressure to give a residue which was purified by prep-HPLC (column: Phenomenex luna C18 150*25 mm*10 μm; mobile phase: [water(HCl)-ACN]; gradient:1%-20% B over 10 min). Compound 6-amino-4,5,6,7-tetrahydro-2-benzothiophene-1-carbonitrile hydrochloride (204) (25.26 mg, 117.64 μmol, 86.18% yield) was obtained as a white solid. LCMS: m/z [M+H]⁺=179.1. ¹H NMR (400 MHz, METHANOL-d₄) δ=7.52 (s, 1H), 3.63 (tdd, J=2.8, 10.4, 13.1 Hz, 1H), 3.38 (dd, J=4.6, 16.8 Hz, 1H), 3.05-2.96 (m, 1H), 2.90-2.78 (m, 2H), 2.29-2.18 (m, 1H), 1.89 (dtd, J=5.6, 11.1, 12.9 Hz, 1H)

### Synthetic scheme for preparation of 3-cyclopropyl-4,5,6,7-tetrahydrobenzothiophen-5-amine hydrochloride (205)

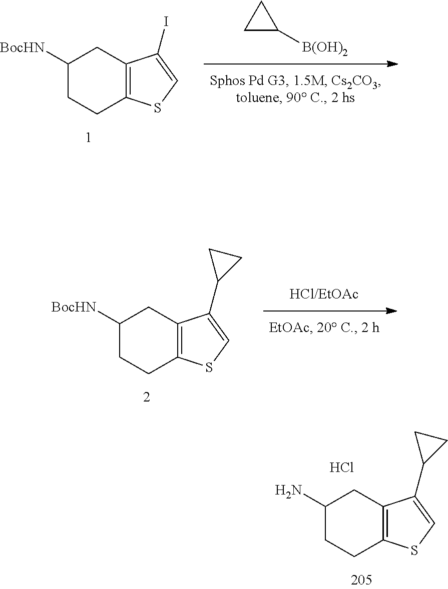 [SMILES: CC(C)(C)OC(=O)NC1CCc2scc(I)c2C1 ; OB(O)C1CC1 ; CC(C)(C)OC(=O)NC1CCc2scc(C3CC3)c2C1 ; NC1CCc2scc(C3CC3)c2C1 ; Cl]

### Procedure for preparation of tert-butyl N-(3-cyclopropyl-4,5,6,7-tetrahydrobenzothiophen-5-yl)carbamate (6)

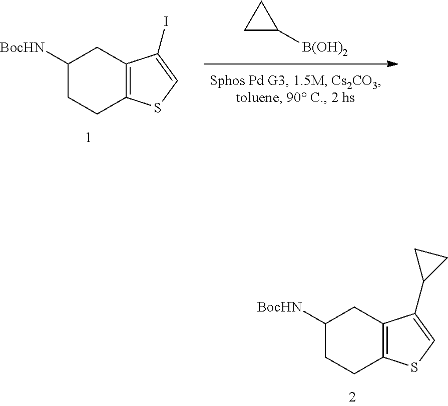 [SMILES: CC(C)(C)OC(=O)NC1CCc2scc(I)c2C1 ; OB(O)C1CC1 ; CC(C)(C)OC(=O)NC1CCc2scc(C3CC3)c2C1]

To a solution of tert-butyl N-(3-iodo-4,5,6,7-tetrahydrobenzothiophen-5-yl)carbamate (1) (0.1 g, 263.67 μmol, 1 eq) in toluene (1.5 mL) was added cyclopropyl boronic acid (67.95 mg, 791.02 μmol, 3 eq), Sphos Pd G3 (20.57 mg, 26.37 μmol, 0.1 eq) and Cs₂CO₃ (1.5 M, 527.35 μL, 3 eq). The mixture was stirred at 90° C. for 2 hr. The reaction mixture was concentrated under reduced pressure to remove solvent. The residue was diluted with EtOAc 20 mL and filtered. The filtrate was concentrated under reduced pressure to give a residue. The residue was purified by prep-HPLC (column: Phenomenex luna C18 150*25 mm*10 μm; mobile phase: [water (TFA)-ACN]; gradient: 56%-86% B over min). Compound tert-butyl N-(3-cyclopropyl-4,5,6,7-tetrahydrobenzothiophen-5-yl)carbamate (2) (58 mg, 197.67 μmol, 74.97% yield) was obtained as a yellow oil. ¹H NMR (400 MHz, CHLOROFORM-d) δ=6.55 (s, 1H), 4.74-4.57 (m, 1H), 4.11-3.98 (m, 1H), 3.02 (dd, J=4.4, 16.1 Hz, 1H), 2.84 (br s, 2H), 2.47 (br dd, J=7.0, 16.3 Hz, 1H), 2.13-2.06 (m, 1H), 1.92-1.77 (m, 1H), 1.68-1.58 (m, 1H), 1.47 (s, 9H), 0.85-0.79 (m, 2H), 0.61-0.49 (m, 2H).

### Procedure for preparation of 3-cyclopropyl-4,5,6,7-tetrahydrobenzothiophen-5-amine hydrochloride (205)

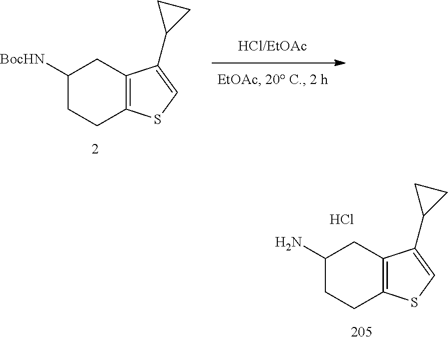 [SMILES: CC(C)(C)OC(=O)NC1CCc2scc(C3CC3)c2C1 ; NC1CCc2scc(C3CC3)c2C1 ; Cl]

To a solution of tert-butyl N-(3-cyclopropyl-4,5,6,7-tetrahydrobenzothiophen-5-yl)carbamate (2) (60 mg, 204.48 μmol, 1 eq) in EtOAc (1 mL) was added HCl/EtOAc (4 M, 1 mL). The mixture was stirred at 20° C. for 2 hr. The reaction mixture was concentrated under vacuum to give a residue. The mixture was purified with prep-HPLC (column: Phenomenex luna C18 150*25 mm*10 μm; mobile phase: [water(HCl)-ACN]; gradient:1%-30% B over 10 min). Compound 3-cyclopropyl-4,5,6,7-tetrahydrobenzothiophen-5-amine hydrochloride (205) (16 mg, 66.15 μmol, 33.47% yield) was obtained as a white solid. ¹H NMR (400 MHz, METHANOL-d₄) δ=6.70 (s, 1H), 3.68-3.55 (m, 1H), 3.22-3.09 (m, 1H), 2.93 (br t, J=5.2 Hz, 2H), 2.72-2.58 (m, 1H), 2.31-2.16 (m, 1H), 1.95 (dddd, J=7.0, 8.6, 10.7, 12.6 Hz, 1H), 1.72-1.60 (m, 1H), 0.93-0.79 (m, 2H), 0.60-0.50 (m, 2H). LC-MS: m/z [M+H]⁺=194.1.

### Synthetic scheme for preparation of 3-cyclobutyl-N-methyl-4,5,6,7-tetrahydrobenzothiophen-5-amine hydrochloride (206)

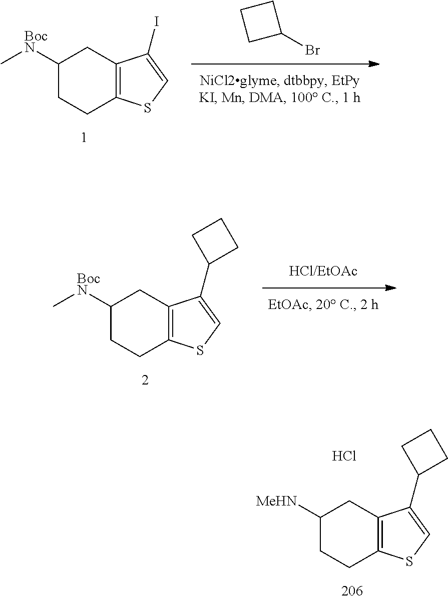 [SMILES: CN(C(=O)OC(C)(C)C)C1CCc2scc(I)c2C1 ; BrC1CCC1 ; CN(C(=O)OC(C)(C)C)C1CCc2scc(C3CCC3)c2C1 ; CNC1CCc2scc(C3CCC3)c2C1 ; Cl]

### Procedure for preparation of tert-butyl N-(3-cyclobutyl-4,5,6,7-tetrahydrobenzothiophen-5-yl)-N-methyl-carbamate (2)

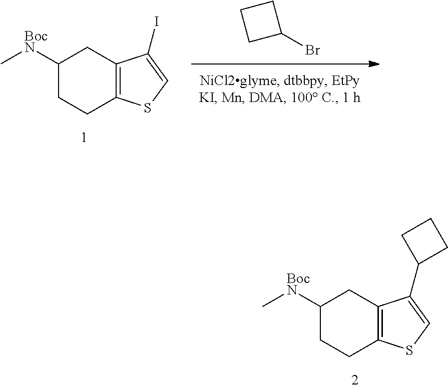 [SMILES: CN(C(=O)OC(C)(C)C)C1CCc2scc(I)c2C1 ; BrC1CCC1 ; CN(C(=O)OC(C)(C)C)C1CCc2scc(C3CCC3)c2C1]

A dried 8 mL vial equipped with a magnetic stir bar was charged with bromocyclobutane (113.28 mg, 839.09 μmol, 79.00 μL, 3 eq), tert-butyl N-(3-iodo-4,5,6,7-tetrahydrobenzothiophen-5-yl)-N-methyl-carbamate (1) (110 mg, 279.70 μmol, 1 eq), 4-tert-butyl-2-(4-tert-butyl-2-pyridyl)pyridine (7.51 mg, 27.97 μmol, 0.1 eq), KI (46.43 mg, 279.70 μmol, 1 eq) and manganese (30.73 mg, 559.39 μmol, 2 eq) in glove box. Then DMA (1.5 mL) and 4-ethylpyridine (29.97 mg, 279.70 μmol, 31.81 μL, 1 eq) was added, followed by NiCl₂.glyme (6.15 mg, 27.97 μmol, 0.1 eq). After addition, the resulting mixture was stirred at 100° C. for 1 h. The reaction mixture was diluted with H₂O (10 mL) and extracted with EtOAc (10 mL*3). The combined organic layers were washed with brine (10 mL*3), dried over anhydrous sodium sulfate, filtered and concentrated under reduced pressure to give a residue. The residue was purified by prep-HPLC (column: Phenomenex luna C18 150*25 mm*10 μm; mobile phase: [water (HCl)-ACN]; gradient: 63%-93% B over 10 min). Compound tert-butyl N-(3-cyclobutyl-4,5,6,7-tetrahydrobenzothiophen-5-yl)-N-methyl-carbamate (2) (38 mg, 98.11 μmol, 35.08% yield) was obtained as a colorless oil. LC-MS: m/z [M+H-tBu]⁺=266.2.

### Procedure for preparation of 3-cyclobutyl-N-methyl-4,5,6,7-tetrahydrobenzothiophen-5-amine hydrochloride (206)

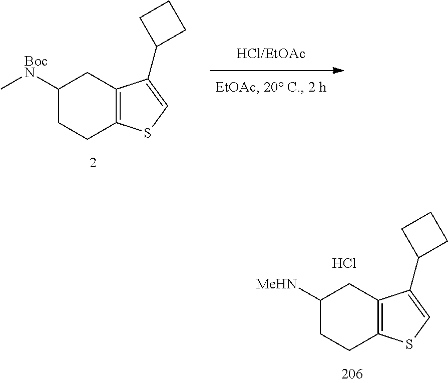 [SMILES: CN(C(=O)OC(C)(C)C)C1CCc2scc(C3CCC3)c2C1 ; CNC1CCc2scc(C3CCC3)c2C1 ; Cl]

To a solution of tert-butyl N-(3-cyclobutyl-4,5,6,7-tetrahydrobenzothiophen-5-yl)-N-methyl-carbamate (2) (38 mg, 118.20 μmol, 1 eq) in EtOAc (1 mL) was added HCl/EtOAc (4 M, 1 mL). The mixture was stirred at 20° C. for 2 hr. The reaction mixture was concentrated under vacuum to give compound 3-cyclobutyl-N-methyl-4,5,6,7-tetrahydrobenzothiophen-5-amine hydrochloride (206) (13.37 mg, 60.19 μmol, 50.92% yield) as a white solid. ¹H NMR (400 MHz, METHANOL-d₄) δ=6.88 (s, 1H), 3.56-3.39 (m, 2H), 3.01 (br dd, J=4.8, 15.7 Hz, 1H), 2.96-2.87 (m, 2H), 2.79 (s, 3H), 2.60-2.49 (m, 1H), 2.42-2.27 (m, 3H), 2.17-1.80 (m, 5H). LC-MS: m/z [M+H]⁺=222.1

### Synthetic scheme for preparation of 3-ethyl-N-methyl-4,5,6,7-tetrahydrobenzothiophen-5-amine hydrochloride (207)

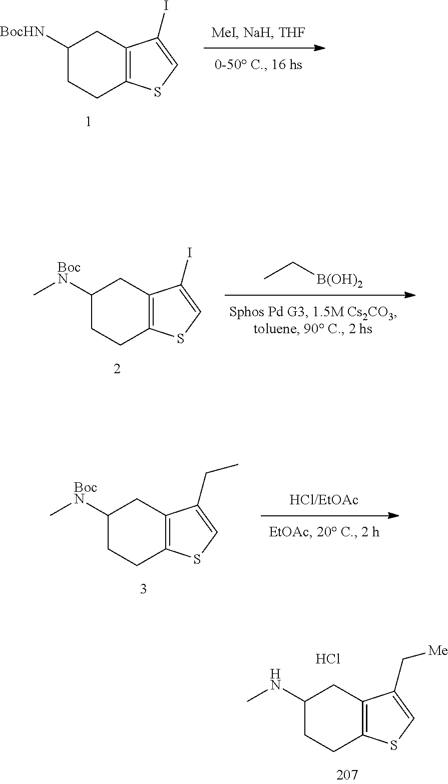

### Procedure for preparation of tert-butyl N-(3-iodo-4,5,6,7-tetrahydrobenzothiophen-5-yl)-N-methyl-carbamate (2)

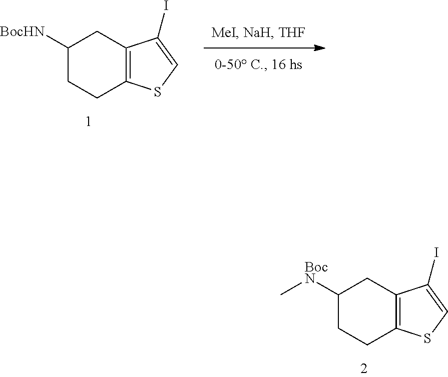 [SMILES: CC(C)(C)OC(=O)NC1CCc2scc(I)c2C1 ; CN(C(=O)OC(C)(C)C)C1CCc2scc(I)c2C1]

To a solution of tert-butyl N-(3-iodo-4,5,6,7-tetrahydrobenzothiophen-5-yl)carbamate (1) (550 mg, 1.45 mmol, 1 eq) in THF (6 mL) was added MeI (329.34 mg, 2.32 mmol, 144.45 μL, 1.6 eq) and NaH (87.00 mg, 2.18 mmol, 60% suspension in mineral oil, 1.5 eq) at 0° C. under N₂. The mixture was stirred at 50° C. for 16 hrs. The reaction mixture was poured into aq. sat. NH₄Cl (10 mL) and water (10 mL), then extracted with EtOAc (10 mL*3). The combined organic phases were washed with brine (15 mL), dried over anhydrous sodium sulfate, filtered and concentrated under vacuum to give a residue. The residue was purified by flash silica gel chromatography (ISCO®; 12 g SepaFlash® Silica Flash Column, eluent 0~20% Ethyl acetate/Petroleum ether gradient @36 mL/min). Compound tert-butyl N-(3-iodo-4,5,6,7-tetrahydrobenzothiophen-5-yl)-N-methyl-carbamate (2) (430 mg, 765.35 μmol, 52.78% yield) was obtained as a yellow oil. ¹H NMR (400 MHz, CHLOROFORM-d) δ=7.13-7.03 (m, 1H), 6.74 (br d, J=5.0 Hz, 1H), 4.62-4.16 (m, 1H), 3.06-2.79 (m, 5H), 2.77-2.59 (m, 1H), 2.56-2.42 (m, 1H), 2.01-1.80 (m, 2H), 1.54-1.45 (m, 9H). LC-MS: m/z [M-tBu+H]⁺=338.1.

### Procedure for preparation of tert-butyl N-(3-ethyl-4,5,6,7-tetrahydrobenzothiophen-5-yl)-N-methyl-carbamate (3)

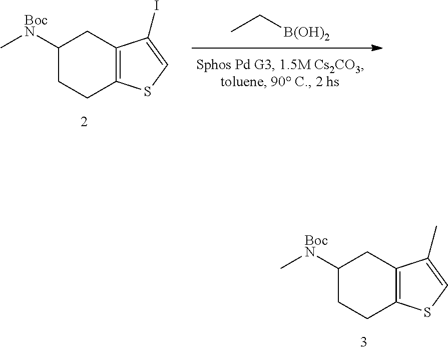

To a solution of tert-butyl N-(3-iodo-4,5,6,7-tetrahydrobenzothiophen-5-yl)-N-methyl-carbamate (2) (90 mg, 228.84 μmol, 1 eq) in toluene (1.5 mL) was added ethyl boronic acid (50.73 mg, 686.53 μmol, 3 eq), SPhos Pd G3 (17.86 mg, 22.88 μmol, 0.1 eq) and aq. Cs₂CO₃ (1.5 M, 3 eq). The mixture was stirred at 90° C. for 2 hr. The mixture was concentrated under reduced pressure to remove solvent. The residue was purified by prep-TLC (SiO2, Petroleum ether/Ethyl acetate=5:1) and then with prep-HPLC (column: Phenomenex luna C18 150*25 mm*10 μm; mobile phase: [water(HCl)-ACN]; gradient:58%-88% B over 10 min). Compound tert-butyl N-(3-ethyl-4,5,6,7-tetrahydrobenzothiophen-5-yl)-N-methyl-carbamate (3) (45 mg, 111.19 μmol, 48.59% yield) was obtained as a colorless oil.

### Procedure for preparation of 3-ethyl-N-methyl-4,5,6,7-tetrahydrobenzothiophen-5-amine hydrochloride (207)

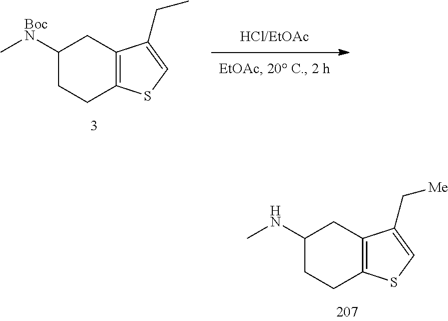 [SMILES: CCc1csc2c1CC(N(C)C(=O)OC(C)(C)C)CC2 ; CCc1csc2c1CC(NC)CC2]

To a solution of tert-butyl N-(3-ethyl-4,5,6,7-tetrahydrobenzothiophen-5-yl)-N-methyl-carbamate (3) (45 mg, 152.32 μmol, 1 eq) in EtOAc (1 mL) was added HCl/EtOAc (4 M, 1 mL). The mixture was stirred at 20° C. for 2 hr. The reaction mixture was concentrated under vacuum to give compound 3-ethyl-N-methyl-4,5,6,7-tetrahydrobenzothiophen-5-amine hydrochloride (207) (27.03 mg, 137.38 μmol, 90.19% yield) as a white solid. ¹H NMR (400 MHz, METHANOL-d₄) δ=6.84 (s, 1H), 3.57-3.46 (m, 1H), 3.07 (dd, J=5.3, 15.6 Hz, 1H), 2.97-2.88 (m, 2H), 2.81 (s, 3H), 2.67-2.57 (m, 1H), 2.55-2.44 (m, 2H), 2.40-2.29 (m, 1H), 1.98 (dddd, J=6.4, 9.6, 10.8, 12.7 Hz, 1H), 1.22 (t, J=7.5 Hz, 3H). LC-MS: m/z [M+H]⁺=196.1.

### Synthetic scheme for preparation of 3-ethyl-4,5,6,7-tetrahydrobenzothiophen-5-amine hydrochloride (208)

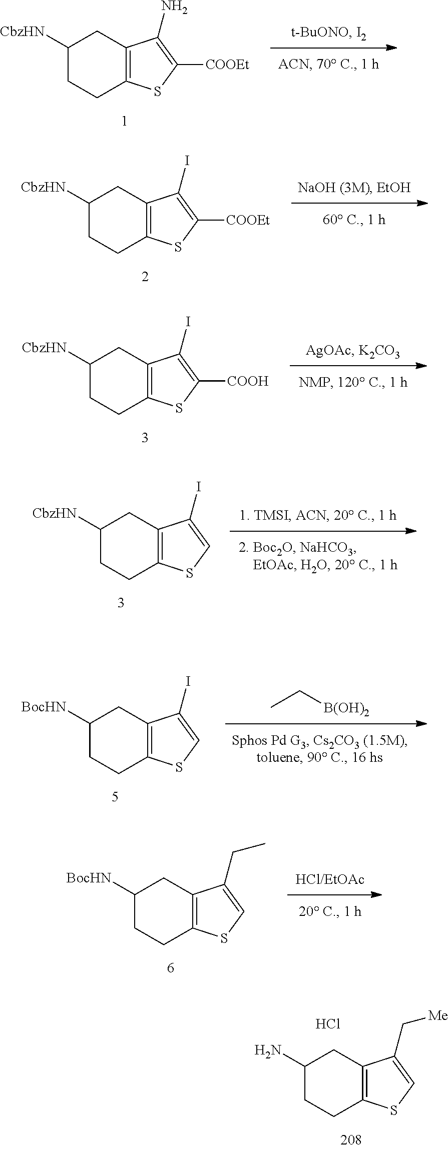

### Procedure for preparation of ethyl 5-(benzyloxycarbonylamino)-3-iodo-4,5,6,7-tetrahydrobenzothiophene-2-carboxylate (2)

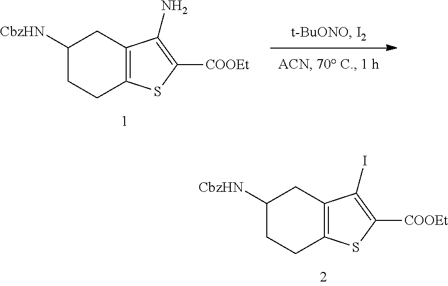 [SMILES: CCOC(=O)c1sc2c(c1N)CC(NC(=O)OCc1ccccc1)CC2 ; CCOC(=O)c1sc2c(c1I)CC(NC(=O)OCc1ccccc1)CC2]

To a solution of ethyl 3-amino-5-(benzyloxycarbonylamino)-4,5,6,7-tetrahydrobenzothiophene-2-carboxylate (1) (3 g, 8.01 mmol, 1 eq) in ACN (30 mL) was added t-BuONO (991.40 mg, 9.61 mmol, 1.14 mL, 1.2 eq) and I₂ (2.44 g, 9.61 mmol, 1.94 mL, 1.2 eq). The mixture was stirred at 70° C. for 1 h. The reaction mixture was concentrated under reduced pressure to give a residue. The residue was diluted with H₂O (90 mL) and extracted with EtOAc (40 mL*3). The combined organic layers were washed with brine (20 mL*3), dried over anhydrous sodium sulfate, filtered and concentrated under reduced pressure. Compound ethyl 5-(benzyloxycarbonylamino)-3-iodo-4,5,6,7-tetrahydrobenzothiophene-2-carboxylate (2) (3.8 g, 6.73 mmol, 84.05% yield) was obtained as a yellow solid. LC-MS: m/z [M+H]⁺=486.1

### Procedure for preparation of 5-(benzyloxycarbonylamino)-3-iodo-4,5,6,7-tetrahydrobenzothiophene-2-carboxylic acid (3)

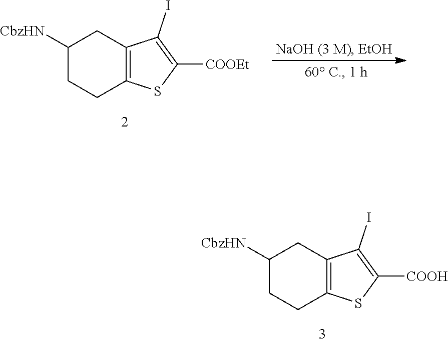 [SMILES: CCOC(=O)c1sc2c(c1I)CC(NC(=O)OCc1ccccc1)CC2 ; "NaOH (3 M), EtOH" ; O=C(NC1CCc2sc(C(=O)O)c(I)c2C1)OCc1ccccc1]

To a solution of ethyl 5-(benzyloxycarbonylamino)-3-iodo-4,5,6,7-tetrahydrobenzothiophene-2-carboxylate (2) (3.8 g, 7.83 mmol, 1 eq) in EtOH (40 mL) was added aq. NaOH (3 M, 7.83 mL, 3 eq). The mixture was stirred at 60° C. for 1 hr. The reaction mixture was concentrated under reduced pressure to remove solvent. The residue was diluted with H₂O (5 mL) and the pH was adjusted to 5-6. The solution was extracted with EtOAc (30 mL*3). The combined organic layers were washed with brine (10 mL*3), dried over anhydrous sodium sulfate, filtered and concentrated under reduced pressure to give compound 5-(benzyloxycarbonylamino)-3-iodo-4,5,6,7-tetrahydrobenzothiophene-2-carboxylic acid (3) (3.3 g, crude) as a yellow solid.

### Procedure for preparation of benzyl N-(3-iodo-4,5,6,7-tetrahydrobenzothiophen-5-yl)carbamate (4)

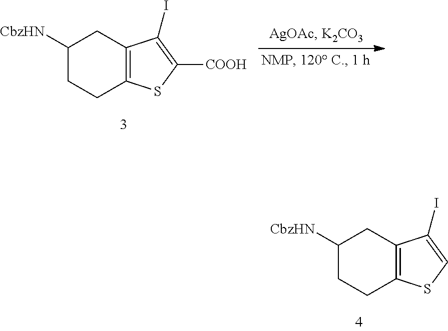 [SMILES: O=C(NC1CCc2sc(C(=O)O)c(I)c2C1)OCc1ccccc1 ; O=C(NC1CCc2scc(I)c2C1)OCc1ccccc1]

To a solution of 5-(benzyloxycarbonylamino)-3-iodo-4,5,6,7-tetrahydrobenzothiophene-2-carboxylic acid (3) (3.3 g, 7.22 mmol, 1 eq) in NMP (35 mL) was added AgOAc (301.13 mg, 1.80 mmol, 0.25 eq) and K₂CO₃ (2.99 g, 21.65 mmol, 3 eq). The mixture was stirred at 120° C. for 1 hr. The reaction mixture was diluted with EtOAc (20 mL) and H₂O and filtered through a pad diatomite earth. The filtrate was diluted with H₂O 30 mL and extracted with EtOAc (30 mL*3). The combined organic layers were washed with brine (15 mL*3), dried over anhydrous sodium sulfate, filtered and concentrated under reduced pressure to give compound benzyl N-(3-iodo-4,5,6,7-tetrahydrobenzothiophen-5-yl)carbamate (4) (2 g, 3.44 mmol, 47.61% yield) as a yellow oil. LC-MS: m/z [M+H]⁺=431.8.

### Procedure for preparation of tert-butyl N-(3-iodo-4,5,6,7-tetrahydrobenzothiophen-5-yl)carbamate (5)

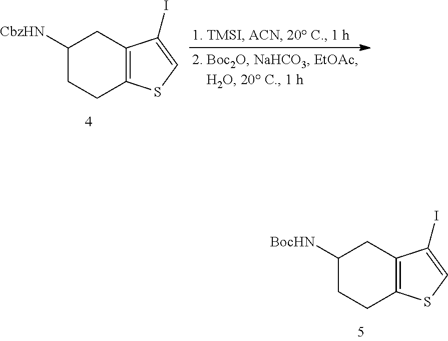 [SMILES: O=C(NC1CCc2scc(I)c2C1)OCc1ccccc1 ; CC(C)(C)OC(=O)NC1CCc2scc(I)c2C1]

To a solution of benzyl N-(3-iodo-4,5,6,7-tetrahydrobenzothiophen-5-yl)carbamate (4) (2.5 g, 6.05 mmol, 1 eq) in ACN (25 mL) was added TMSI (3.63 g, 18.15 mmol, 2.47 mL, 3 eq). The mixture was stirred at 20° C. for 1 hr. The reaction mixture was concentrated under reduced pressure to remove solvent. The residue was diluted with H₂O (10 mL), and then it was added 1 M HCl to adjust the solution pH to 5-6. Then the mixture was extracted with EtOAc (10 mL). To the aqueous phase it was added saturated aq. NaHCO₃ (1.02 g, 12.10 mmol, 470.77 μL, 2 eq) to adjust the pH to7-8 and (Boc)₂O (1.58 g, 7.26 mmol, 2 mL, 1.2 eq) in EtOAc (10 mL). The mixture was stirred at 20° C. for 1 h. The reaction mixture was extracted with EtOAc (10 mL*3). The combined organic layers were washed with brine (30 mL), dried over anhydrous sodium sulfate, filtered and concentrated under reduced pressure to give a residue. The residue was purified by flash silica gel chromatography (ISCO®; 20 g SepaFlash® Silica Flash Column, eluent 0~15% Ethyl acetate/Petroleum ether gradient @50 mL/min). Compound tert-butyl N-(3-iodo-4,5,6,7-tetrahydrobenzothiophen-5-yl)carbamate (5) (0.9 g, 1.31 mmol, 21.58% yield) was obtained as a white solid. ¹H NMR (400 MHz, CHLOROFORM-d) δ=7.30-7.27 (m, 1H), 4.61 (br dd, J=1.7, 4.8 Hz, 1H), 4.16-3.93 (m, 1H), 2.96-2.78 (m, 3H), 2.33 (dd, J=7.4, 16.3 Hz, 1H), 2.15-2.01 (m, 1H), 1.90-1.75 (m, 1H), 1.47 (s, 9H). LCMS: m/z [M-tBu+H]⁺=324.0.

### Procedure for preparation of tert-butyl N-(3-ethyl-4,5,6,7-tetrahydrobenzothiophen-5-yl)carbamate (6)

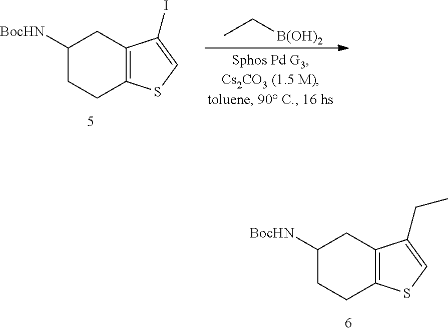

To a solution of tert-butyl N-(3-iodo-4,5,6,7-tetrahydrobenzothiophen-5-yl)carbamate (5) (0.13 g, 342.78 μmol, 1 eq) in toluene (1.5 mL) was added ethyl boronic acid (75.98 mg, 1.03 mmol, 3 eq), Sphos Pd G3 (26.75 mg, 34.28 μmol, 0.1 eq) and aq. Cs₂CO₃ (1.5 M, 3 eq). The mixture was stirred at 90° C. for 16 hrs. The reaction mixture was concentrated under reduced pressure and then diluted with EtOAc (10 mL) and filtered. The filtrate was concentrated under reduced pressure to give a residue. The residue was purified by pre-HPLC (column: Phenomenex Luna C18 150*25 mm*10 μm; mobile phase: [water (TFA)-ACN]; gradient: 56%-86% B). Compound tert-butyl N-(3-ethyl-4,5,6,7-tetrahydrobenzothiophen-5-yl)carbamate (6) (63 mg, 223.87 μmol, 65.31% yield) was obtained as a yellow oil.

### Procedure for preparation of 3-ethyl-4,5,6,7-tetrahydrobenzothiophen-5-amine hydrochloride (208)

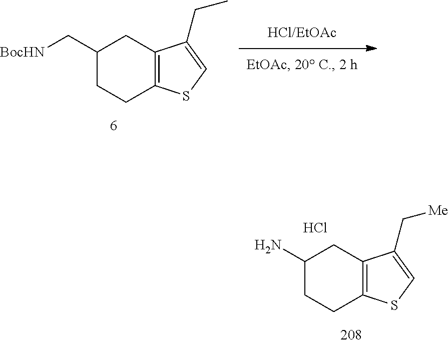 [SMILES: CCc1csc2c1CC(CNC(=O)OC(C)(C)C)CC2 ; CCc1csc2c1CC(N)CC2 ; Cl]

To a solution of tert-butyl N-(3-ethyl-4,5,6,7-tetrahydrobenzothiophen-5-yl)carbamate (6) (70 mg, 248.74 μmol, 1 eq) in EtOAc (1 mL) was added HCl/EtOAc (4 M, 1.75 mL, 28.14 eq). The mixture was stirred at 20° C. for 2 hr. The reaction mixture was concentrated under vacuum to give a residue which was purified with prep-HPLC (column: Phenomenex luna C18 150*25 mm*10 μm; mobile phase: [water(HCl)-ACN]; gradient:1%-30% B over 10 min). Compound 3-ethyl-4,5,6,7-tetrahydrobenzothiophen-5-amine hydrochloride (208) (10.37 mg, 45.72 μmol, 20.42% yield) was obtained as a white solid. ¹H NMR (400 MHz, METHANOL-d₄) δ=6.86 (s, 1H), 3.65-3.54 (m, 1H), 3.05-2.89 (m, 3H), 2.60-2.42 (m, 3H), 2.30-2.19 (m, 1H), 2.01-1.88 (m, 1H), 1.22 (t, J=7.5 Hz, 3H). LC-MS: m/z [M+H]⁺=182.0.

### Synthetic scheme for preparation of 3-fluoro-4,5,6,7-tetrahydrobenzothiophen-6-amine hydrochloride (209)

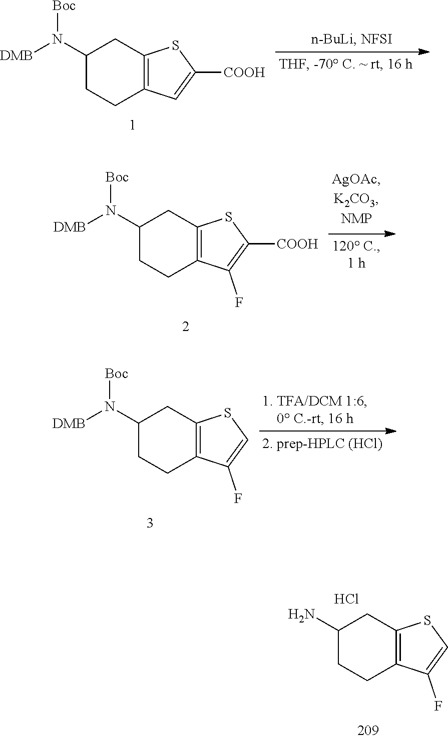

### Procedure for preparation of 6-[tert-butoxycarbonyl-[(2,4-dimethoxyphenyl)methyl]amino]-3-fluoro-4,5,6,7-tetrahydrobenzothiophene-2-carboxylic acid (2)

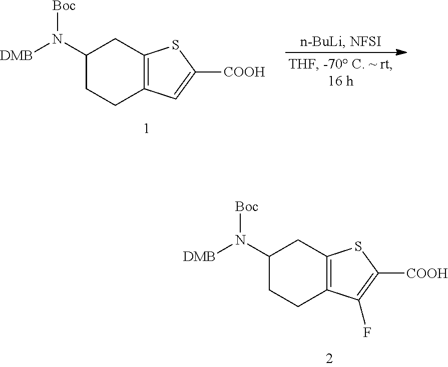

To the mixture of 6-[tert-butoxycarbonyl-[(2,4-dimethoxyphenyl)methyl]amino]-4,5,6,7-tetrahydrobenzothiophene-2-carboxylic acid (1) (1 g, 2.23 mmol, 1.00 eq) in THF (10 mL) was added n-BuLi (2.5 M, 2.2 mL, 2.40 eq) at −70° C. under nitrogen atmosphere. After stirring for 0.5 h at −70° C., NFSI (845 mg, 2.68 mmol, 1.20 eq) was added in portions and the resulting mixture was allowed to warm to 15-20° C. and stirred for 16 h. The reaction mixture was quenched by sat. NH₄Cl (20 mL), diluted with water (20 mL) and extracted with EtOAc (15 mL*3). The combined organic phases were washed with brine (30 mL), dried over anhydrous sodium sulfate, filtered and concentrated under vacuum to give 6-[tert-butoxycarbonyl-[(2,4-dimethoxyphenyl)methyl]amino]-3-fluoro-4,5,6,7-tetrahydrobenzothiophene-2-carboxylic acid (2) (1.5 g, crude) as a yellow gum which was used into next step without further purification.

### Procedure for preparation of tert-butyl N-[(2,4-dimethoxyphenyl)methyl]-N-(3-fluoro-4,5,6,7-tetrahydrobenzothiophen-6-yl)carbamate (3)

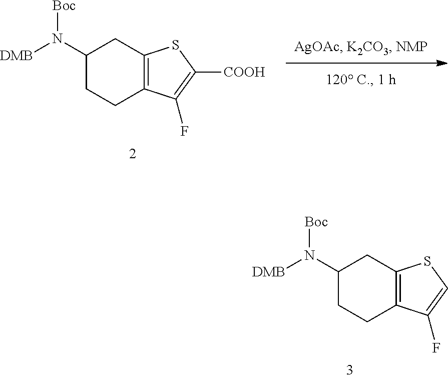

The mixture of 6-[tert-butoxycarbonyl-[(2,4-dimethoxyphenyl)methyl]amino]-3-fluoro-4,5,6,7-tetrahydrobenzothiophene-2-carboxylic acid (2) (1.3 g, 2.79 mmol, 1 eq), AgOAc (140 mg, 838 μmol, 0.30 eq) and K₂CO₃ (1.16 g, 8.38 mmol, 3.00 eq) in NMP (15 mL) was stirred at 120° C. for 1 h. The reaction mixture was diluted with EtOAc (20 mL) and filtered. The filtrate was diluted with water (80 mL) and extracted with EtOAc (20 mL*3). The combined organic phases were washed with brine (30 mL), dried over anhydrous sodium sulfate, filtered and concentrated under vacuum to give a residue. The residue was purified by flash silica gel chromatography (ISCO®; 12 g SepaFlash® Silica Flash Column, eluent 0~10% Ethyl acetate/Petroleum ether gradient @36 mL/min) to give tert-butyl N-[(2,4-dimethoxyphenyl)methyl]-N-(3-fluoro-4,5,6,7-tetrahydrobenzothiophen-6-yl)carbamate (3) (260 mg, 617 μmol, 22.1% yield) as a yellow oil. ¹H NMR (400 MHz, CDCl₃-d) δ=7.15 (br d, J=6.3 Hz, 1H), 6.53-6.39 (m, 3H), 4.52-4.31 (m, 2H), 3.82 (d, J=9.4 Hz, 6H), 2.84-2.45 (m, 5H), 1.90 (br d, J=1.0 Hz, 2H), 1.52-1.41 (m, 9H). LCMS: m/z [M-tBu+H]⁺=366.1.

### Procedure for preparation of 3-fluoro-4,5,6,7-tetrahydrobenzothiophen-6-amine hydrochloride (209)

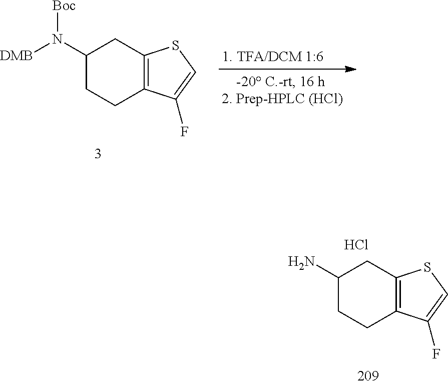

To the solution of tert-butyl N-[(2,4-dimethoxyphenyl)methyl]-N-(3-fluoro-4,5,6,7-tetrahydrobenzothiophen-6-yl)carbamate (3) (260 mg, 616.81 μmol, 1 eq) in DCM (6 mL) was added TFA (1.54 g, 13.46 mmol, 1 mL, 21.83 eq) at −20° C. After addition, the mixture was warm to 10-15° C. slowly and stirred for 16 h. The reaction mixture was poured into sat. NaHCO₃ (40 mL) and extracted with EtOAc (15 mL*3). The combined organic phases were dried over anhydrous sodium sulfate, filtered and concentrated under vacuum to give a residue. The residue was purified by prep-HPLC (column: Phenomenex luna C18 150*25 mm*10 μm; mobile phase: [water(HCl)-ACN]; gradient:1%-25% B over 10 min) to give 3-fluoro-4,5,6,7-tetrahydrobenzothiophen-6-amine hydrochloride (209) (32.31 mg, 150 μmol, 24.3% yield) as a white solid. ¹H NMR (400 MHz, MeOD-d₄) δ=6.73 (s, 1H), 3.68-3.58 (m, 1H), 3.16 (dd, J=5.1, 16.0 Hz, 1H), 2.87-2.58 (m, 3H), 2.27-2.16 (m, 1H), 1.93-1.87 (m, 1H). ¹⁹F NMR (400 MHz, MeOD-d₄) δ=134.65. LCMS: m/z [M+H]⁺=172.0.

### Synthetic scheme for preparation 1-fluoro-N, 3-dimethyl-4,5,6,7-tetrahydro-2-benzothiophen-5-amine hydrochloride enantiomer 1 (210) and enantiomer 2 (211)

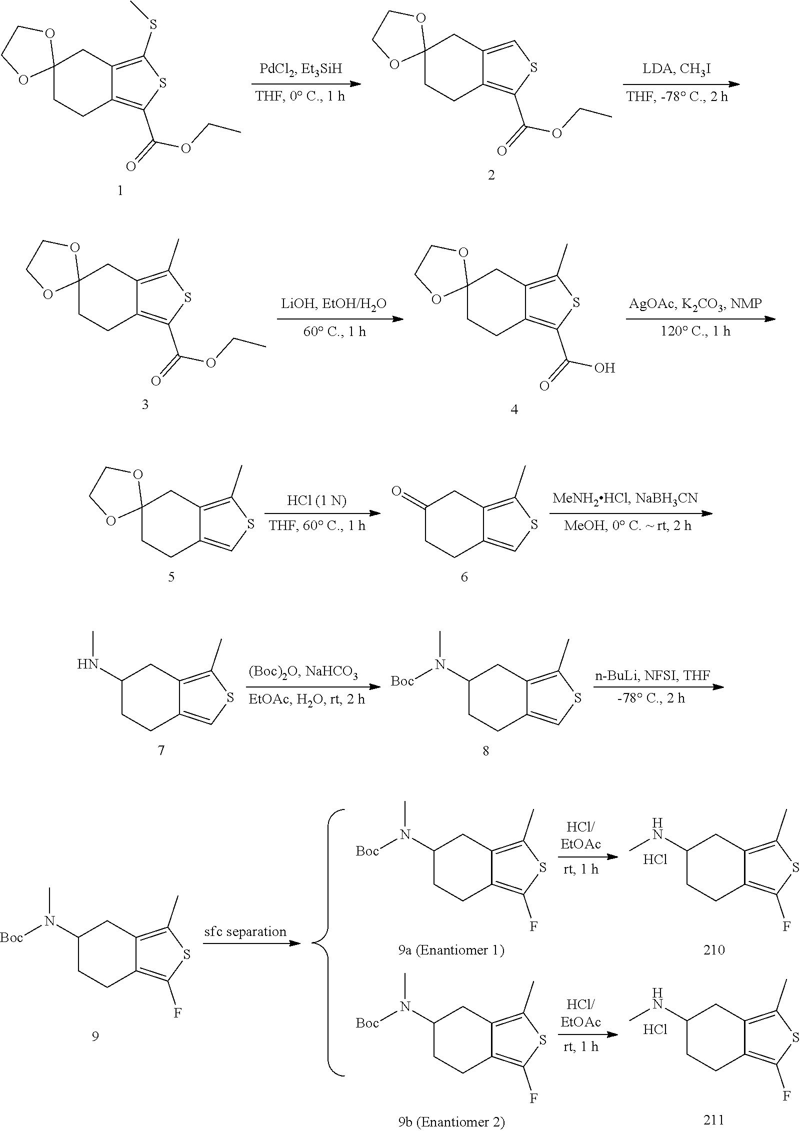

### Procedure for preparation of ethyl spiro[1,3-dioxolane-2,5′-6,7-dihydro-4H-2-benzothiophene]-1′-carboxylate(2)

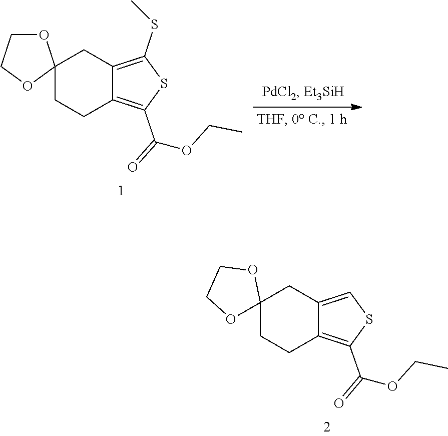 [SMILES: CCOC(=O)c1sc(SC)c2c1CCC1(C2)OCCO1 ; CCOC(=O)c1scc2c1CCC1(C2)OCCO1]

To a solution of ethyl 3′-methylsulfanylspiro[1,3-dioxolane-2,5′-6,7-dihydro-4H-2-benzothiophene]-1′-carboxylate (1) (10 g, 31.80 mmol, 1 eq) in THF (150 mL) was added PdCl₂ (451.19 mg, 2.54 mmol, 0.08 eq) and Et₃SiH (5.55 g, 47.71 mmol, 7.62 mL, 1.5 eq) at 0° C. The mixture was stirred at 0° C. for 1 hr. The reaction mixture was diluted with aq. sat. NaHCO₃ (100 mL) and extracted with EtOAc (100 mL*2). The combined organic layers were washed with aq. sat. NaCl (100 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~12% Ethyl acetate/Petroleum ether gradient @40 mL/min). This afforded a mixture of ethyl spiro[1,3-dioxolane-2,5′-6,7-dihydro-4H-2-benzothiophene]-1′-carboxylate (2) (10 g, crude) and ethyl 5-oxo-6,7-dihydro-4H-2-benzothiophene-1-carboxylate as a yellow oil. LCMS: m/z [M+H]⁺=269.2.

### Procedure for preparation of ethyl 3′-methylspiro[1,3-dioxolane-2,5′-6,7-dihydro-4H-2-benzothiophene]-1′-carboxylate (3)

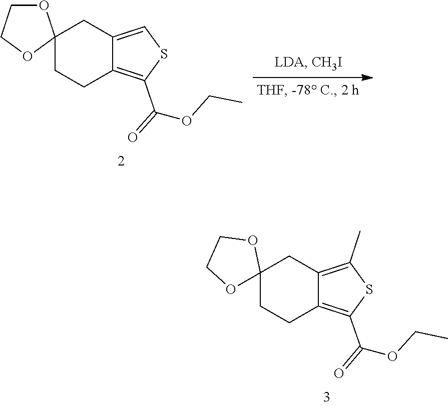 [SMILES: CCOC(=O)c1scc2c1CCC1(C2)OCCO1 ; CCOC(=O)c1sc(C)c2c1CCC1(C2)OCCO1]

To a solution of ethyl spiro[1,3-dioxolane-2,5′-6,7-dihydro-4H-2-benzothiophene]-1′-carboxylate (2) (5.5 g, 20.50 mmol, 1 eq) in THF (60 mL) was added dropwise LDA (2 M, 15.37 mL, 1.5 eq) at −78° C. After addition, the mixture was stirred at −78° C. for 0.5 hr, and then MeI (4.36 g, 30.75 mmol, 1.91 mL, 1.5 eq) was added dropwise at−78° C. The resulting mixture was stirred at −78° C. for 1.5 hr. The reaction mixture was quenched with aq. sat.NH₄Cl (50 mL) at 0° C., and extracted with EtOAc (30 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 120 g SepaFlash® Silica Flash Column, eluent 0~30% Ethyl acetate/Petroleum ether gradient @40 mL/min). Compound ethyl 3′-methylspiro[1,3-dioxolane-2,5′-6,7-dihydro-4H-2-benzothiophene]-1′-carboxylate (3) (5.1 g, 18.06 mmol, 88.12% yield) was obtained as a yellow oil. ¹H NMR (400 MHz, CHLOROFORM-d) δ=4.29 (q, J=7.0 Hz, 2H), 4.04 (s, 4H), 3.19 (t, J=6.8 Hz, 2H), 2.75 (s, 2H), 2.32 (s, 3H), 1.92 (t, J=6.8 Hz, 2H), 1.35 (t, J=7.1 Hz, 3H). LCMS: m/z [M+H]+=283.3.

### Procedure for preparation of 3′-methylspiro[1,3-dioxolane-2,5′-6,7-dihydro-4H-2-benzothiophene]-1′-carboxylic acid (4)

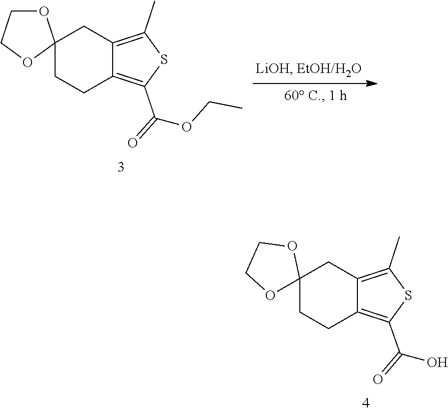 [SMILES: CCOC(=O)c1sc(C)c2c1CCC1(C2)OCCO1 ; Cc1sc(C(=O)O)c2c1CC1(CC2)OCCO1]

To a solution of ethyl 3′-methylspiro[1,3-dioxolane-2,5′-6,7-dihydro-4H-2-benzothiophene]-1′-carboxylate (3) (5.1 g, 18.06 mmol, 1 eq) in EtOH (25 mL) and H₂O (25 mL) was added LiOH·H₂O (3.03 g, 72.25 mmol, 4 eq). The mixture was stirred at 60° C. for 1 hr. The reaction mixture was concentrated under vacuum to remove EtOH, extracted with EtOAc (100 mL) and separated. The aqueous phase was adjusted pH to ~7 with HCl (1 mol/L in water), then adjusted pH to 3-4 with citric acid, filtered. The filter cake was washed with water (20 mL) and collected and then dried under vacuum to give 3′-methylspiro[1,3-dioxolane-2,5′-6,7-dihydro-4H-2-benzothiophene]-1′-carboxylic acid (4) (4.3 g, 16.91 mmol, 93.61% yield) as a white solid. ¹H NMR (400 MHz, CHLOROFORM-d) 6=7.53 (s, 1H), 4.09-4.00 (m, 4H), 3.03 (t, J=6.5 Hz, 2H), 2.88 (s, 2H), 2.02 (t, J=6.6 Hz, 2H)

### Procedure for preparation of 1′-methylspiro[1,3-dioxolane-2,6′-5,7-dihydro-4H-2-benzothiophene](5)

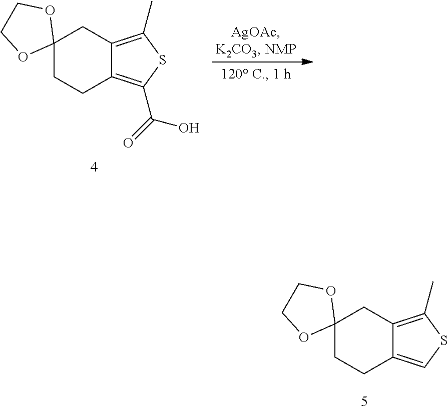 [SMILES: Cc1sc(C(=O)O)c2c1CC1(CC2)OCCO1 ; Cc1scc2c1CC1(CC2)OCCO1]

To a solution of 3′-methylspiro[1,3-dioxolane-2,5′-6,7-dihydro-4H-2-benzothiophene]-1′-carboxylic acid (4) (4.3 g, 16.91 mmol, 1 eq) in NMP (40 mL) was added K₂CO₃ (7.01 g, 50.73 mmol, 3 eq) and silver acetate (1.41 g, 8.45 mmol, 0.5 eq). The mixture was stirred at 120° C. for 1 hr. The reaction mixture was diluted with H₂O (100 mL) and extracted with EtOAc (100 mL*3). The combined organic layers were washed with aq. sat. NaCl (100 mL*3), dried over anhydrous Na₂SO₄, filtered and concentrated under reduced pressure to give a residue. The residue was purified by flash silica gel chromatography (ISCO®; 20 g SepaFlash® Silica Flash Column, eluent 0~17% Ethyl acetate/Petroleum ether gradient @40 mL/min). Compound 1′-methylspiro[1,3-dioxolane-2,6′-5,7-dihydro-4H-2-benzothiophene](5) (3 g, 14.27 mmol, 84.37% yield) was obtained as a yellow solid. LCMS: m/z [M+H]⁺=211.2. ¹H NMR (400 MHz, CHLOROFORM-d) δ=6.67 (s, 1H), 4.07-4.01 (m, 4H), 2.86 (t, J=6.7 Hz, 2H), 2.77 (s, 2H), 2.30 (s, 3H), 1.91 (t, J=6.8 Hz, 2H)

### Procedure for preparation of 3-methyl-6,7-dihydro-4H-2-benzothiophen-5-one (6)

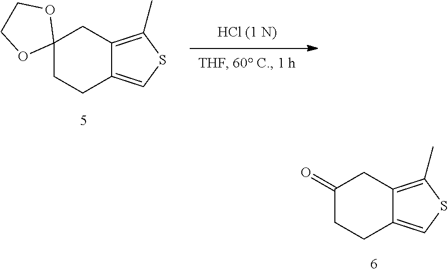

To a solution of 1′-methylspiro[1,3-dioxolane-2,6′-5,7-dihydro-4H-2-benzothiophene](5) (2 g, 9.51 mmol, 1 eq) in THF (20 mL) was added HCl (1 M, 20 mL, 2.10 eq). The mixture was stirred at 60° C. for 1 hr. The reaction mixture was diluted with aq. sat. NaHCO₃ (10 mL) and extracted with EtOAc (30 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 20 g SepaFlash® Silica Flash Column, eluent 0~7% Ethyl acetate/Petroleum ether gradient @40 mL/min) to give 3-methyl-6,7-dihydro-4H-2-benzothiophen-5-one (6) (1 g, 6.02 mmol, 63.25% yield) as a yellow oil. LCMS: m/z [M+H]⁺=167.0. ¹H NMR (400 MHz, CHLOROFORM-d) δ=6.78 (s, 1H), 3.43 (s, 2H), 3.02-2.92 (m, 2H), 2.61-2.54 (m, 2H), 2.33 (s, 3H)

### Procedure for preparation of N, 3-dimethyl-4,5,6,7-tetrahydro-2-benzothiophen-5-amine (7)

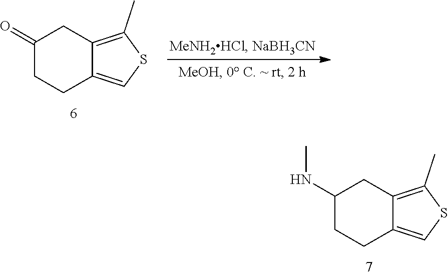

To a mixture of 3-methyl-6,7-dihydro-4H-2-benzothiophen-5-one (6) (1 g, 6.02 mmol, 1 eq) in MeOH (40 mL) was added methylamine hydrochloride (1.22 g, 18.05 mmol, 3 eq) under N₂. The mixture was stirred at 25° C. for 30 min. Then to the mixture was added sodium cyanoborohydride (1.89 g, 30.08 mmol, 5 eq) at 0° C. and stirred at 0° C. for 1.5 hours. The reaction was treated with water (5 ml). The mixture was concentrated in vacuo. Compound N, 3-dimethyl-4,5,6,7-tetrahydro-2-benzothiophen-5-amine (7) (1 g, crude) was obtained as a yellow solid. LCMS: m/z [M+H]⁺=181.9

### Procedure for preparation of tert-butyl N-methyl-N-(3-methyl-4,5,6,7-tetrahydro-2-benzothiophen-5-yl)carbamate (8)

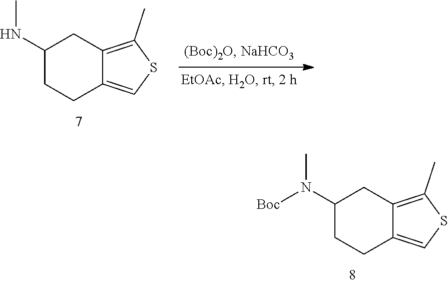 [SMILES: CNC1CCc2csc(C)c2C1 ; Cc1scc2c1CC(N(C)C(=O)OC(C)(C)C)CC2]

To a solution of N, 3-dimethyl-4,5,6,7-tetrahydro-2-benzothiophen-5-amine (7) (1 g, 5.52 mmol, 1 eq) in EtOAc (10 mL) and H₂O (10 mL) was added NaHCO₃ (926.73 mg, 11.03 mmol, 429.24 μL, 2 eq) and (Boc)₂O (1.81 g, 8.27 mmol, 1.90 mL, 1.5 eq). The mixture was stirred at 25° C. for 2 hr. The reaction mixture was diluted with H₂O (50 mL) and extracted with EtOAc (30 mL*2). The combined organic layers were washed with aq. sat.NaCl (30 mL), dried over anhydrous Na₂SO₄, filtered and concentrated under reduced pressure to give a residue. The residue was purified by flash silica gel chromatography (ISCO®; 20 g SepaFlash® Silica Flash Column, eluent 0~12% Ethyl acetate/Petroleum ether gradient @40 mL/min). Compound tert-butyl N-methyl-N-(3-methyl-4,5,6,7-tetrahydro-2-benzothiophen-5-yl)carbamate (8) (1 g, 3.55 mmol, 64.42% yield) was obtained as a colorless oil. LCMS: m/z [M+H-tBu]⁺=226.1. ¹H NMR (400 MHz, CHLOROFORM-d) δ=6.64 (s, 1H), 4.53-4.16 (m, 1H), 2.92-2.71 (m, 6H), 2.57-2.43 (m, 1H), 2.31 (s, 3H), 1.91-1.74 (m, 2H), 1.49 (s, 9H)

### Procedure for preparation of tert-butyl N-(1-fluoro-3-methyl-4,5,6,7-tetrahydro-2-benzothiophen-5-yl)-N-methyl-carbamate (9)

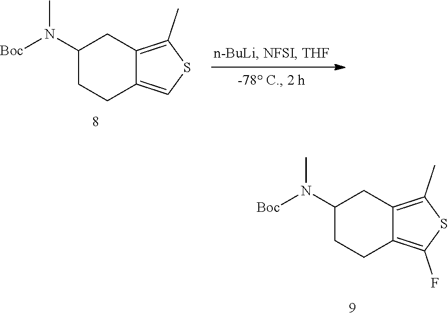 [SMILES: Cc1scc2c1CC(N(C)C(=O)OC(C)(C)C)CC2 ; Cc1sc(F)c2c1CC(N(C)C(=O)OC(C)(C)C)CC2]

To a solution of tert-butyl N-methyl-N-(3-methyl-4,5,6,7-tetrahydro-2-benzothiophen-5-yl)carbamate (8) (1 g, 3.55 mmol, 1 eq) in THF (10 mL) was added dropwise n-BuLi (2.5 M, 2.13 mL, 1.5 eq) at −78° C. After addition, the mixture was stirred at −78° C. for 1 hr, and then NFSI (1.57 g, 4.97 mmol, 1.4 eq) in THF (10 mL) was added dropwise at −78° C. The resulting mixture was stirred at −78° C. for 2 hr. The reaction mixture was diluted with H₂O (10 mL) and extracted with EtOAc (30 mL*2). The combined organic layers were washed with aq. sat.NaCl (10 mL), dried over anhydrous Na₂SO₄, filtered and concentrated under reduced pressure to give a residue. The residue was purified by prep-HPLC (column: Phenomenex luna C18 150*40 mm*15 μm; mobile phase: [water(FA)-ACN]; gradient:60%-90% B over 15 min). Compound tert-butyl N-(1-fluoro-3-methyl-4,5,6,7-tetrahydro-2-benzothiophen-5-yl)-N-methyl-carbamate (9) (200 mg, 668.00 μmol, 18.80% yield) was obtained as a white solid. LCMS: m/z [M+H-tBu]⁺=244.2

### Procedure for preparation of tert-butyl N-(1-fluoro-3-methyl-4,5,6,7-tetrahydro-2-benzothiophen-5-yl)-N-methyl-carbamate enantiomer 1 (9a) and enantiomer 2 (9b)

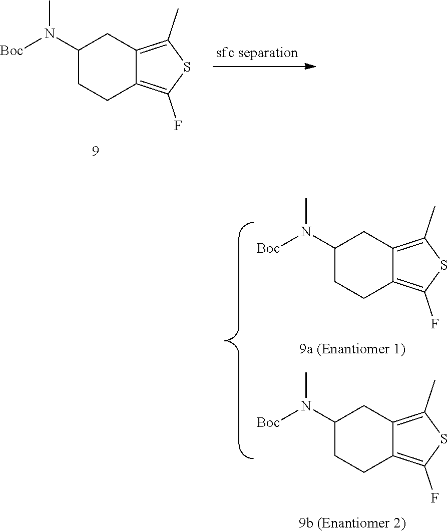 [SMILES: Cc1sc(F)c2c1CC(N(C)C(=O)OC(C)(C)C)CC2 ; Cc1sc(F)c2c1CC(N(C)C(=O)OC(C)(C)C)CC2 ; Cc1sc(F)c2c1CC(N(C)C(=O)OC(C)(C)C)CC2]

Racemic tert-butyl N-(1-fluoro-3-methyl-4,5,6,7-tetrahydro-2-benzothiophen-5-yl)-N-methyl-carbamate (9) was separated by SFC to enantiomers (column: DAICEL CHIRALPAK IG (250 mm*50 mm, 10 μm); mobile phase: [CO₂-i-PrOH (0.1% NH₃H₂O)]; B %:10%, isocratic elution mode). to afford enantiomer 1 (9a) (RT=0.793 min, 50 mg) and enantiomer 2 (9b) (RT=0.842 min, 70 mg). Compound tert-butyl N-(1-fluoro-3-methyl-4,5,6,7-tetrahydro-2-benzothiophen-5-yl)-N-methyl-carbamate enantiomer 1 (9a) (50 mg, 167.00 μmol, 25.00% yield) was obtained as a yellow solid. Compound tert-butyl N-(1-fluoro-3-methyl-4,5,6,7-tetrahydro-2-benzothiophen-5-yl)-N-methyl-carbamate enantiomer 2 (9b) (70 mg, 233.80 μmol, 35.00% yield) was obtained as a yellow solid.

### Procedure for preparation of 1-fluoro-N, 3-dimethyl-4,5,6,7-tetrahydro-2-benzothiophen-5-amine hydrochloride enantiomer 1 (210)

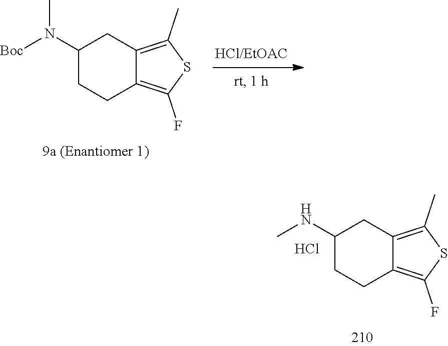 [SMILES: Cc1sc(F)c2c1CC(N(C)C(=O)OC(C)(C)C)CC2 ; CNC1CCc2c(F)sc(C)c2C1 ; Cl]

To a solution of tert-butyl N-(1-fluoro-3-methyl-4,5,6,7-tetrahydro-2-benzothiophen-5-yl)-N-methyl-carbamate enantiomer 1 (9a) (50 mg, 167.00 μmol, 1 eq) in EtOAc (2 mL) was added HCl/EtOAc (4 M, 2 mL, 47.90 eq). The mixture was stirred at 25° C. for 1 hr. The reaction mixture was filtered and the cake was washed with EtOAc (10 mL). The residue was purified by prep-HPLC (column: Phenomenex luna C18 150*25 mm*10 μm; mobile phase: [water(HCl)-ACN]; gradient:5%-35% B over 10 min). Compound 1-fluoro-N, 3-dimethyl-4,5,6,7-tetrahydro-2-benzothiophen-5-amine hydrochloride enantiomer 1 (210) (27.09 mg, 110.28 μmol, 66.04% yield, 95.97% purity, HCl) was obtained as a yellow solid. LCMS: m/z [M+H]⁺=200.1. H NMR (400 MHz, METHANOL-d4) δ=3.46-3.38 (m, 1H), 3.17-3.06 (m, 1H), 2.91-2.82 (m, 1H), 2.79 (s, 3H), 2.67-2.42 (m, 2H), 2.30-2.16 (m, 4H), 1.85-1.67 (m, 1H)

### Procedure for preparation of 1-fluoro-N, 3-dimethyl-4,5,6,7-tetrahydro-2-benzothiophen-5-amine hydrochloride enantiomer 2 (211)

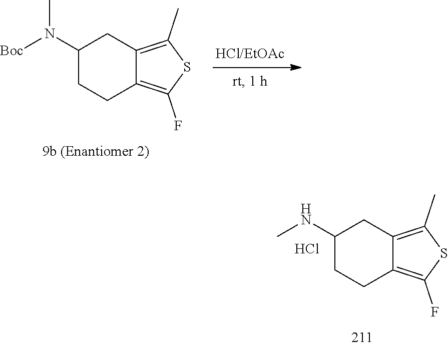 [SMILES: Cc1sc(F)c2c1CC(N(C)C(=O)OC(C)(C)C)CC2 ; CNC1CCc2c(F)sc(C)c2C1 ; Cl]

To a solution of tert-butyl N-(1-fluoro-3-methyl-4,5,6,7-tetrahydro-2-benzothiophen-5-yl)-N-methyl-carbamate enantiomer 2 (9b) (70 mg, 233.80 μmol, 1 eq) in EtOAc (2 mL) was added HCl/EtOAc (4 M, 2 mL, 34.22 eq). The mixture was stirred at 25° C. for 1 hr. The reaction mixture was then filtered and the cake was washed with EtOAc (10 mL). The residue was purified by prep-HPLC (column: Phenomenex luna C18 150*25 mm*10 μm; mobile phase: [water(HCl)-ACN]; gradient:5%-35% B over 10 min). Compound 1-fluoro-N, 3-dimethyl-4,5,6,7-tetrahydro-2-benzothiophen-5-amine hydrochloride enantiomer 2 (211) (28.49 mg, 118.83 μmol, 50.83% yield) was obtained as an off-white solid. LCMS: m/z [M+H]⁺=200.1. ¹H NMR (400 MHz, METHANOL-d4) δ=3.46-3.36 (m, 1H), 3.17-3.07 (m, 1H), 2.92-2.82 (m, 1H), 2.79 (s, 3H), 2.68-2.43 (m, 2H), 2.29-2.16 (m, 4H), 1.84-1.69 (m, 1H)

### Synthetic scheme for preparation of 4,5,6,7-tetrahydrobenzothiophen-5-amine hydrochloride enantiomer 1 (212) and enantiomer 2 (213)

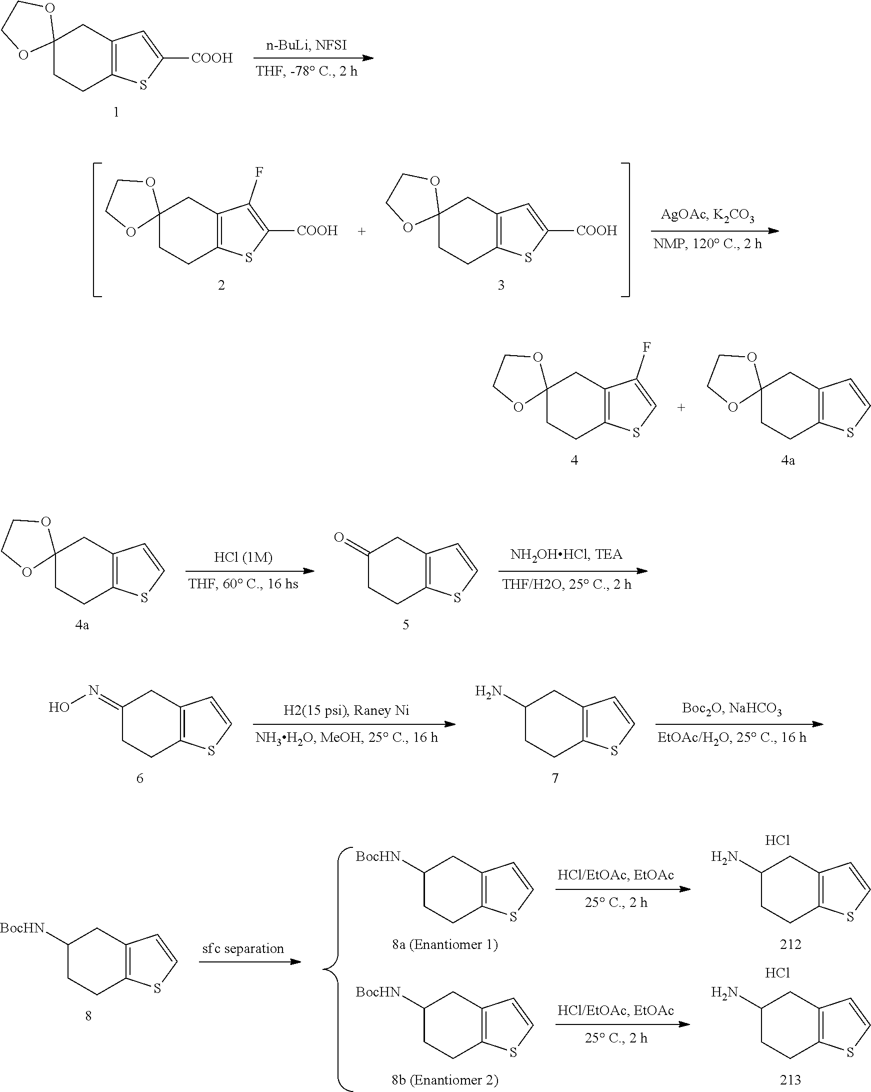

### Isolation of 6,7-dihydro-4H-spiro[benzo[b]thiophene-5,2′-[1,3]dioxolane](4a)

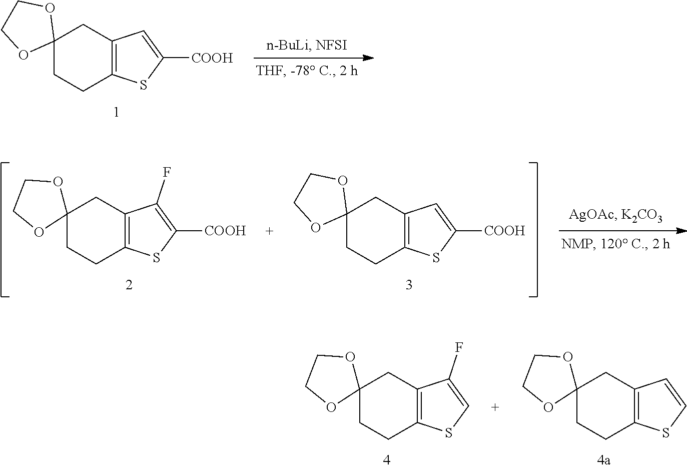

Intermediate (4a) was isolated as byproduct of the fluorination/decarboxylation reaction of spiro[1,3-dioxolane-2,5′-6,7-dihydro-4H-benzothiophene]-2′-carboxylic acid (1) as described below.

To a solution of spiro[1,3-dioxolane-2,5′-6,7-dihydro-4H-benzothiophene]-2′-carboxylic acid (10 g, 41.62 mmol, 1 eq) in THF (100 mL) was added dropwise n-BuLi (2.5 M, 41.62 mL, 2.5 eq) at −78° C. After addition, the mixture was stirred at −78° C. for 1 hr, and then NFSI (18.37 g, 58.27 mmol, 1.4 eq) was added at −78° C. The resulting mixture was stirred at −78° C. for 1 hr. The reaction mixture was quenched with aq. sat.NH₄Cl 30 mL, and diluted with water 50 mL. The pH was about 8-9. The mixture was extracted with EtOAc (50 mL*2). Then the aqueous phase pH was adjusted to 5-6 with citric acid and the water layer extracted with EtOAc (50 mL*2). The combined organic layer from this extraction was washed with aq. sat.NaCl (10 mL), dried over anhydrous Na₂SO₄, filtered and concentrated under reduced pressure to give a residue a mixture of compounds (2) and (3) (11 g, crude) as a red solid.This material was dissolved in NMP (100 mL). Followed addition of silver acetate (3.55 g, 21.30 mmol, 0.5 eq) and K₂CO₃ (17.66 g, 127.78 mmol, 3 eq). The mixture was stirred at 120° C. for 2 hr then cooled and diluted with H₂O (300 mL) and extracted with EtOAc (100 mL*2). The combined organic layers were washed with aq. sat.NaCl (100 mL), dried over anhydrous Na₂SO₄, filtered and concentrated under reduced pressure. The residue was purified by flash silica gel chromatography (ISCO®; 4 g SepaFlash® Silica Flash Column, eluent 0~8% Ethyl acetate/Petroleum ether gradient @40 mL/min) to give 3′-fluorospiro[1,3-dioxolane-2,5′-6,7-dihydro-4H-benzothiophene](4) (1.2 g, 5.60 mmol, 13.15% yield) as a colorless oil. LCMS: m/z [M+H]⁺=215.0. ¹H NMR (400 MHz, CHLOROFORM-d) δ=6.49 (s, 1H), 4.08-4.02 (m, 4H), 2.91 (t, J=6.4 Hz, 2H), 2.74 (s, 2H), 1.99 (t, J=6.5 Hz, 2H) and compound spiro[1,3-dioxolane-2,5′-6,7-dihydro-4H-benzothiophene](4a) (2.9 g, 14.78 mmol, 34.69% yield) d as a colorless oil. 1H NMR (400 MHz, CHLOROFORM-d) δ=7.10 (d, J=5.1 Hz, 1H), 6.73 (d, J=5.0 Hz, 1H), 4.04 (d, J=1.1 Hz, 4H), 2.98 (br t, J=6.4 Hz, 2H), 2.87 (s, 2H), 2.01 (t, J=6.5 Hz, 2H)

### Procedure for preparation of 6,7-dihydro-4H-benzothiophen-5-one (5)

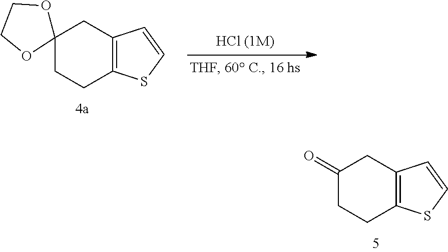 [SMILES: c1cc2c(s1)CCC1(C2)OCCO1 ; O=C1CCc2sccc2C1]

To a solution of spiro[1,3-dioxolane-2,5′-6,7-dihydro-4H-benzothiophene](4a) (1.85 g, 9.43 mmol, 1 eq) in THF (20 mL) was added HCl (1 M, 20 mL, 2.12 eq). The mixture was stirred at 60° C. for 16 hr. The mixture was quenched by addition NaHCO₃ (100 mL) extracted with EtOAc (30 mL*3). The combined organic layers were washed with aq. sat. NaCl (15 mL*2), dried over Na₂SO₄, filtered and concentrated under reduced pressure to give a residue. The residue was purified by flash silica gel chromatography (ISCO®; 12 g SepaFlash® Silica Flash Column, eluent 0~10% Ethyl acetate/Petroleum ether gradient @40 mL/min). Compound 6,7-dihydro-4H-benzothiophen-5-one (5) (1.8 g, 8.75 mmol, 92.84% yield, 74% purity) was obtained as a yellow oil. LC-MS: m/z [M+H]⁺=153.1.

### Procedure for preparation of 6,7-dihydro-4H-benzothiophen-5-one oxime (6)

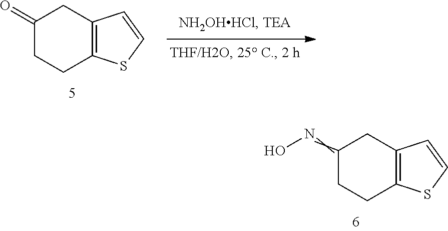 [SMILES: O=C1CCc2sccc2C1 ; ON=C1CCc2sccc2C1]

To a solution of 6,7-dihydro-4H-benzothiophen-5-one (5) (1.8 g, 11.83 mmol, 1 eq) in THF (20 mL) and H₂O (20 mL) was added hydroxylamine hydrochloride (1.64 g, 23.65 mmol, 2 eq) and TEA (2.39 g, 23.65 mmol, 3.29 mL, 2 eq). The mixture was stirred at 25° C. for 2 hr. The reaction mixture was quenched by addition of aq. NH₄Cl (30 mL), and then diluted with EtOAc (60 mL) and extracted with H₂O (30 mL). The combined organic layers were washed with brine (20 mL), dried over anhydrous sodium sulfate, filtered and concentrated under reduced pressure to give a residue. The residue was purified by flash silica gel chromatography (ISCO®; 20 g SepaFlash® Silica Flash Column, eluent 0~20% Ethyl acetate/Petroleum ether gradient @50 mL/min). Compound 6,7-dihydro-4H-benzothiophen-5-one oxime (6) (2 g, 11.00 mmol, 93.04% yield) was obtained as a yellow oil. LC-MS: m/z [M+1]⁺=168.3.

### Procedure for preparation of 4,5,6,7-tetrahydrobenzothiophen-5-amine (4)

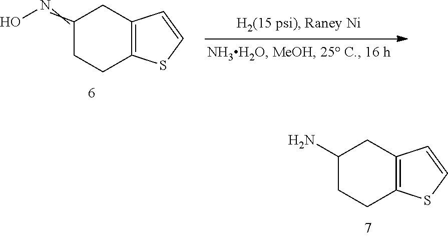 [SMILES: ON=C1CCc2sccc2C1 ; NC1CCc2sccc2C1]

To a solution of Raney-Ni (1.1 g, 12.84 mmol, 1.07 eq) in MeOH (5 mL) was added a solution of 6,7-dihydro-4H-benzothiophen-5-one oxime (6) (2 g, 11.96 mmol, 1 eq) and NH₃·H₂O (0.2 mL) under N₂. The suspension was degassed under vacuum and purged with H₂ several times. The mixture was stirred under H₂ (15 psi) at 25° C. for 16 hours. The reaction mixture was then filtered and the filtrate was concentrated and was diluted with H₂O (15 mL) and extracted with EtOAc 30 (10 mL*3). The combined organic layers were washed with brine (10 mL*3), dried over anhydrous sodium sulfate, filtered and concentrated under reduced pressure to give a residue. The residue was purified by prep-HPLC (column: Waters Xbridge 150*25 mm*5 μm; mobile phase: [water (ammonia hydroxide v/v)-ACN]; gradient:18%-48% B over min). Compound 4,5,6,7-tetrahydrobenzothiophen-5-amine (7) (1.4 g, 9.14 mmol, 76.39% yield) was obtained as a gray solid.

### Procedure for preparation of tert-butyl N-(4,5,6,7-tetrahydrobenzothiophen-5-yl)carbamate (8)

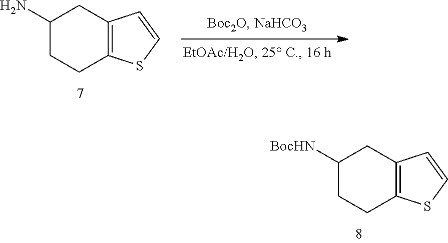 [SMILES: NC1CCc2sccc2C1 ; CC(C)(C)OC(=O)NC1CCc2sccc2C1]

To a solution of 4,5,6,7-tetrahydrobenzothiophen-5-amine (7) (200 mg, 1.31 mmol, 1 eq) in EtOAc (1 mL) was added NaHCO₃ (109.64 mg, 1.31 mmol, 1 eq) and water and (Boc)₂O (341.80 mg, 1.57 mmol, 359.79 μL, 1.2 eq). The mixture was stirred at 25° C. for 16 hr. The reaction mixture was then concentrated under vacuum to give a residue. The residue was purified by flash silica gel chromatography (ISCO®; 4 g SepaFlash® Silica Flash Column, eluent 0~15% Ethyl acetate/Petroleum ether gradient @36 mL/min). Compound tert-butyl N-(4,5,6,7-tetrahydrobenzothiophen-5-yl)carbamate (8) (135 mg, 522.18 μmol, 40.01% yield, 98% purity) was obtained as a white solid.

### Procedure for preparation of tert-butyl N-(4,5,6,7-tetrahydrobenzothiophen-5-yl)carbamate enantiomer 1 (8a) and enantiomer 2 (8b)

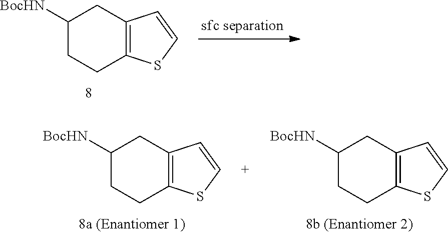 [SMILES: CC(C)(C)OC(=O)NC1CCc2sccc2C1 ; CC(C)(C)OC(=O)NC1CCc2sccc2C1 ; CC(C)(C)OC(=O)NC1CCc2sccc2C1]

Racemic tert-butyl N-(4,5,6,7-tetrahydrobenzothiophen-5-yl)carbamate (8) (135 mg, 522.18 mol) was separated by SFC (column: DAICEL CHIRALCEL OX (250 mm*30 mm, 10 μm); mobile phase: [CO₂-EtOH (0.1% NH₃H₂O)]; B %:10%, isocratic elution mode). 1^{st} peak in SFC: compound tert-butyl N-(4,5,6,7-tetrahydrobenzothiophen-5-yl)carbamate enantiomer 1 (8a) (60 mg, 229.71 μmol, 43.11% yield), obtained as a white solid. LC-MS: m/z [M+H-tBu]⁺=198.0. 2^{nd} peak in SFC: compound tert-butyl N-(4,5,6,7-tetrahydrobenzothiophen-5-yl)carbamate enantiomer 2 (8b) (64 mg, 250.08 μmol, 46.93% yield), obtained as a white solid. LC-MS: m/z [M+H-tBu]⁺=198.0

### Procedure for preparation of 4,5,6,7-tetrahydrobenzothiophen-5-amine hydrochloride enantiomer 1 (212)

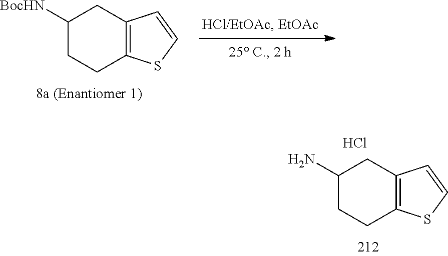 [SMILES: CC(C)(C)OC(=O)NC1CCc2sccc2C1 ; Cl ; NC1CCc2sccc2C1]

To a solution of tert-butyl N-(4,5,6,7-tetrahydrobenzothiophen-5-yl)carbamate (8a) (60 mg, 236.82 μmol, 1 eq) in EtOAc (1 mL) was added HCl/EtOAc (4 M, 2 mL, 33.78 eq). The mixture was stirred at 25° C. for 2 hr. The reaction mixture was concentrated under vacuum to give compound 4,5,6,7-tetrahydrobenzothiophen-5-amine hydrochloride (212) (23.85 mg, 149.53 μmol, 63.14% yield) as an off-white solid. LC-MS: m/z [M+H]⁺=154.3. ¹H NMR (400 MHz, METHANOL-d₄) δ=7.22 (d, J=5.3 Hz, 1H), 6.79 (d, J=5.1 Hz, 1H), 3.67-3.51 (m, 1H), 3.10 (dd, J=4.9, 15.8 Hz, 1H), 3.03-2.91 (m, 2H), 2.68 (br dd, J=9.1, 15.7 Hz, 1H), 2.30-2.19 (m, 1H), 2.01-1.89 (m, 1H)

### Procedure for preparation of 4,5,6,7-tetrahydrobenzothiophen-5-amine hydrochloride enantiomer 2 (213)

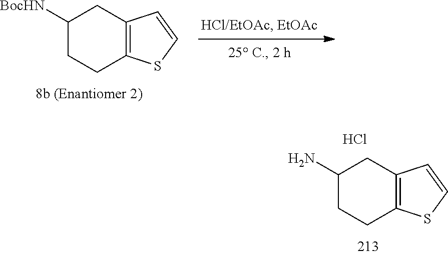 [SMILES: CC(C)(C)OC(=O)NC1CCc2sccc2C1 ; Cl ; NC1CCc2sccc2C1]

To a solution of tert-butyl N-(4,5,6,7-tetrahydrobenzothiophen-5-yl)carbamate enantiomer 2 (8b) (64 mg, 252.61 μmol, 1 eq) in EtOAc (1 mL) was added HCl/EtOAc (4 M, 2 mL, 31.67 eq). The mixture was stirred at 25° C. for 2 hr. The reaction mixture was concentrated under vacuum to compound 4,5,6,7-tetrahydrobenzothiophen-5-amine hydrochloride enantiomer 2 (213) (24.51 mg, 153.58 μmol, 60.80% yield) as an off-white solid. LC-MS: m/z [M+H]⁺=154.4. ¹H NMR (400 MHz, METHANOL-d₄) δ=7.22 (d, J=5.1 Hz, 1H), 6.79 (d, J=5.1 Hz, 1H), 3.67-3.51 (m, 1H), 3.10 (dd, J=5.4, 16.0 Hz, 1H), 3.04-2.88 (m, 2H), 2.67 (br dd, J=9.3, 15.6 Hz, 1H), 2.31-2.20 (m, 1H), 2.02-1.88 (m, 1H).

### Synthetic scheme for preparation of 3-fluoro-2-methyl-4,5,6,7-tetrahydrobenzothiophen-5-amine hydrochloride (214)

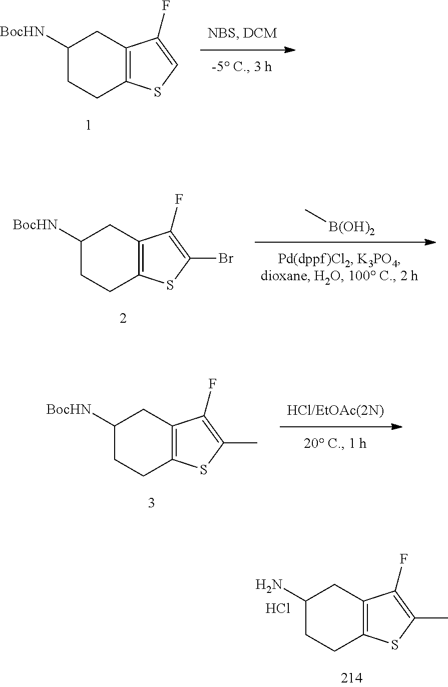

### Procedure for preparation of tert-butyl N-(2-bromo-3-fluoro-4,5,6,7-tetrahydrobenzothiophen-5-yl)carbamate (2)

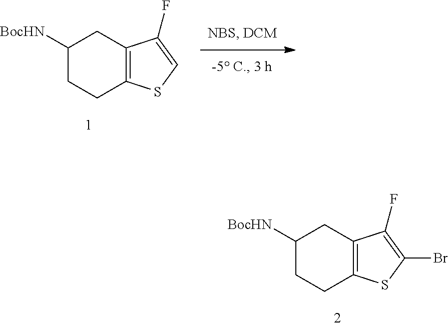 [SMILES: CC(C)(C)OC(=O)NC1CCc2scc(F)c2C1 ; CC(C)(C)OC(=O)NC1CCc2sc(Br)c(F)c2C1]

To a solution of tert-butyl N-(3-fluoro-4,5,6,7-tetrahydrobenzothiophen-5-yl)carbamate (1) (150 mg, 552.79 μmol, 1 eq) in DCM (3 mL) was added AcOH (3.15 g, 52.40 mmol, 3.00 mL, 94.80 eq) and NBS (118.07 mg, 663.35 μmol, 1.2 eq). The mixture was stirred at −5° C. for 3 hr. The reaction mixture was diluted with H₂O (10 mL) and extracted with EtOAc (10 mL*2). The combined organic layers were washed with aq. sat. NaCl (10 mL), dried over anhydrous Na₂SO₄, filtered and concentrated under reduced pressure to give a residue. The residue was purified by prep-TLC (Petroleum ether:Ethyl acetate=3:1). Compound tert-butyl N-(2-bromo-3-fluoro-4,5,6,7-tetrahydrobenzothiophen-5-yl)carbamate (2) (70 mg) was obtained as a yellow solid. LC-MS: m/z [M+H-tBu]⁺=294.1.

### Procedure for preparation of tert-butyl N-(3-fluoro-2-methyl-4,5,6,7-tetrahydrobenzothiophen-5-yl)carbamate (3)

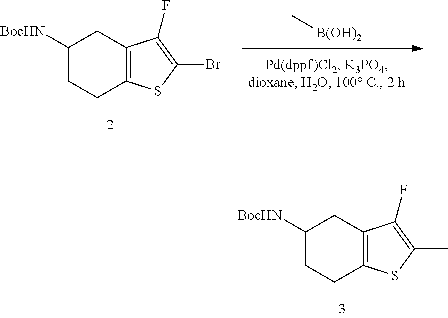

A mixture of tert-butyl N-(2-bromo-3-fluoro-4,5,6,7-tetrahydrobenzothiophen-5-yl)carbamate (2) (70 mg, 199.86 μmol, 1 eq), methylboronic acid (59.82 mg, 999.30 μmol, 5 eq), Pd(dppf)Cl₂ (7.31 mg, 9.99 μmol, 0.05 eq) and K₃PO₄ (127.27 mg, 599.58 μmol, 3 eq) in dioxane (2 mL) and H₂O (0.4 mL) was degassed and purged with N₂ for 3 times, and then the mixture was stirred at 100° C. for 2 hr under N₂ atmosphere. The reaction mixture was diluted with H₂O (5 mL) and extracted with EtOAc (5 mL*2). The combined organic layers were washed with aq. sat.NaCl (10 mL), dried over anhydrous Na₂SO₄, filtered and concentrated under reduced pressure to give a residue. The residue was purified by prep-HPLC (column: Phenomenex luna C18 150*25 mm*10 μm; mobile phase: [water(FA)-ACN]; gradient:62%-92% B over 7 min). Compound tert-butyl N-(3-fluoro-2-methyl-4,5,6,7-tetrahydrobenzothiophen-5-yl)carbamate (3) (12 mg, 42.05 μmol, 21.04% yield) was obtained as a white solid. LC-MS: m/z [M+H-Bu]⁺=230.1.

### Procedure for 3-fluoro-2-methyl-4,5,6,7-tetrahydrobenzothiophen-5-amine hydrochloride (214)

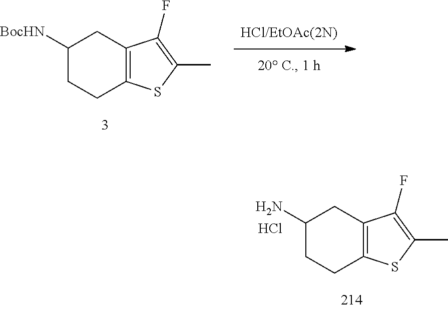 [SMILES: Cc1sc2c(c1F)CC(NC(=O)OC(C)(C)C)CC2 ; Cl ; Cc1sc2c(c1F)CC(N)CC2]

To a solution of tert-butyl N-(3-fluoro-2-methyl-4,5,6,7-tetrahydrobenzothiophen-5-yl)carbamate (3) (12 mg, 42.05 μmol, 1 eq) in EtOAc (2 mL) was added HCl/EtOAc (4 M, 2 mL, 190.25 eq). The mixture was stirred at 25° C. for 1 hr and then concentrated to give a residue. Compound 3-fluoro-2-methyl-4,5,6,7-tetrahydrobenzothiophen-5-amine hydrochloride (214) (5.13 mg, 22.24 μmol, 52.88% yield) was obtained as an off-white solid. LC-MS: m/z [M+H]⁺=186.4. ¹H NMR (400 MHz, METHANOL-d4) δ=3.63-3.53 (m, 1H), 2.94 (dd, J=5.4, 15.9 Hz, 1H), 2.83 (br t, J=6.3 Hz, 2H), 2.48 (dd, J=9.2, 15.8 Hz, 1H), 2.29-2.17 (m, 4H), 1.92 (tdd, J=7.8, 10.7, 12.9 Hz, 1H)

### Synthetic scheme for preparation of 3-chloro-6,7-dihydro-5H-thieno[3,2-b]pyran-6-amine hydrochloride (215)

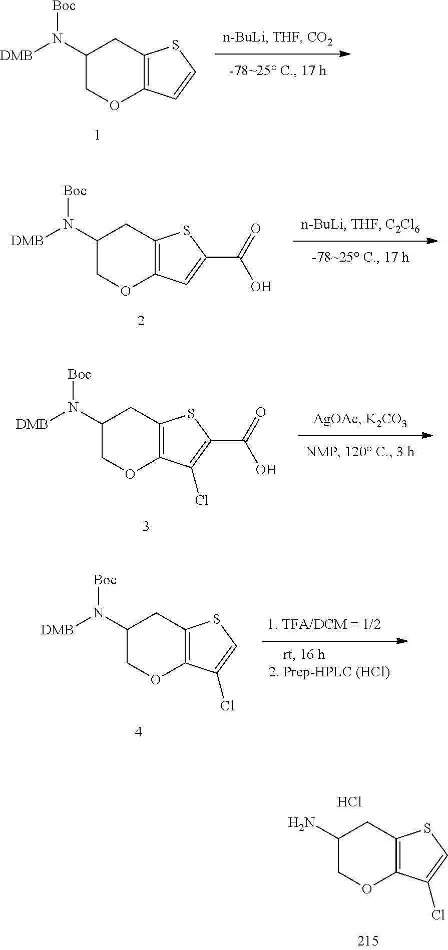

### Procedure for preparation of 6-[tert-butoxycarbonyl-[(2,4-dimethoxyphenyl)methyl]amino]-6,7-dihydro-5H-thieno[3,2-b]pyran-2-carboxylic acid (2)

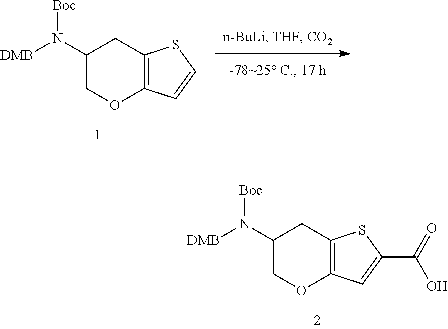

To a solution of tert-butyl N-(6,7-dihydro-5H-thieno[3,2-b]pyran-6-yl)-N-[(2,4-dimethoxyphenyl)methyl]carbamate (1) (1 g, 2.47 mmol, 1 eq) in THF (15 mL) was added dropwise n-BuLi (2.5 M, 2.37 mL, 2.4 eq) at −78° C. over 0.5 hr. After addition, the mixture was stirred at this temperature for 0.5 hr, and then CO₂ (gas) was bubbled into the solution at −78° C. The resulting mixture was allowed to warm up to 25° C. and stirred for 16 hr. The reaction mixture was poured into aq. NH₄Cl saturated solution (20 mL) and extracted with EtOAc (30 mL*3). The combined organic phases were washed with brine (10 mL), dried over anhydrous sodium sulfate, filtered and concentrated under vacuum to give a residue. The residue was purified by flash silica gel chromatography (ISCO®; 25 g SepaFlash® Silica Flash Column, eluent 0~50% Ethyl acetate/Petroleum ether gradient @60 mL/min) to afford 6-[tert-butoxycarbonyl-[(2,4-dimethoxyphenyl)methyl]amino]-6,7-dihydro-5H-thieno[3,2-b]pyran-2-carboxylic acid (2) (320 mg, 711.88 μmol, 28.87% yield) as a yellow solid. ¹H NMR (400 MHz, DMSO-d6) δ=13.21-12.84 (m, 1H), 7.16 (s, 1H), 7.05 (d, J=8.3 Hz, 1H), 6.54 (d, J=2.3 Hz, 1H), 6.52-6.46 (m, 1H), 4.41-4.21 (m, 2H), 4.21-4.02 (m, 3H), 3.74 (d, J=1.9 Hz, 6H), 3.10-2.96 (m, 1H), 2.84 (br dd, J=5.3, 16.6 Hz, 1H), 1.46-1.31 (m, 9H).

### Procedure for preparation of 6-((tert-butoxycarbonyl)(3,4-dimethylbenzyl)amino)-3-chloro-6,7-dihydro-5H-thieno[3,2-b]pyran-2-carboxylic acid (3)

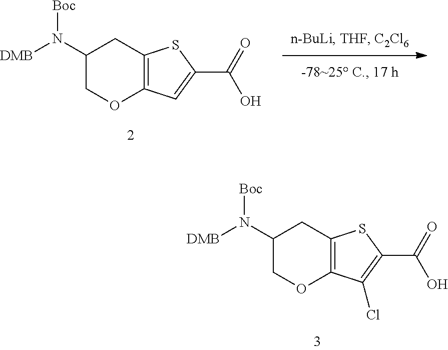

To a solution of 6-[tert-butoxycarbonyl-[(2,4-dimethoxyphenyl)methyl]amino]-6,7-dihydro-5H-thieno[3,2-b]pyran-2-carboxylic acid (2) (300 mg, 667.38 μmol, 1 eq) in THF (5 mL) was added dropwise n-BuLi (2.5 M, 640.69 μL, 2.4 eq) at −78° C. over 0.5 hr. After addition, the mixture was stirred at this temperature for 0.5 hr, and then 1,1,1,2,2,2-hexachloroethane (236.99 mg, 1.00 mmol, 113.34 μL, 1.5 eq) in THF (1 mL) was added dropwise at −78° C. The resulting mixture was warmed up slowly and stirred at 25° C. for 16 hr. The reaction mixture was poured into aq. NH₄Cl saturated solution (30 mL) and extracted with EtOAc (20 mL*3). The combined organic phases were washed with brine (10 mL), dried over anhydrous sodium sulfate, filtered and concentrated under vacuum to give a residue. The residue was purified by prep-HPLC (column: Phenomenex luna C18 150*25 mm*10 μm; mobile phase: [water(HCl)-ACN]; gradient:40%-70% B over 10 min) to afford 6-((tert-butoxycarbonyl)(3,4-dimethylbenzyl)amino)-3-chloro-6,7-dihydro-5H-thieno[3,2-b]pyran-2-carboxylic acid (3) (80 mg, 165.30 μmol, 24.77% yield) as a yellow solid. ¹H NMR (400 MHz, DMSO-d6) δ=13.20 (br d, J=2.3 Hz, 1H), 7.04 (d, J=8.3 Hz, 1H), 6.56-6.47 (m, 2H), 4.47-4.06 (m, 5H), 3.74 (d, J=2.5 Hz, 6H), 3.13-2.98 (m, 1H), 2.91-2.81 (m, 1H), 1.51-1.27 (m, 9H).

### Procedure for preparation of tert-butyl N-(3-chloro-6,7-dihydro-5H-thieno[3,2-b]pyran-6-yl)-N-[(2,4-dimethoxyphenyl)methyl]carbamate (4)

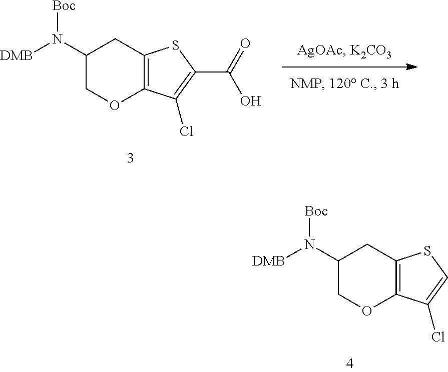

To a solution of 6-((tert-butoxycarbonyl)(3,4-dimethylbenzyl)amino)-3-chloro-6,7-dihydro-5H-thieno[3,2-b]pyran-2-carboxylic acid (3) (80 mg, 165.30 μmol, 1 eq) in NMP (1 mL) was added AgOAc (5.52 mg, 33.06 μmol, 0.2 eq) and K₂CO₃ (68.54 mg, 495.91 μmol, 3 eq). The mixture was stirred at 120° C. for 3 hr. The reaction mixture was poured into H₂O (10 mL) and extracted with EtOAc (20 mL*3). The combined organic phases were washed with brine (10 mL), dried over anhydrous sodium sulfate, filtered and concentrated under vacuum to give a residue. The residue was purified by prep-HPLC (column: Phenomenex Gemini NX C18 150×30 mm, 3 μm mobile phase: [water(FA)-ACN]; gradient:70%-100% B over 7 min) to afford tert-butyl N-(3-chloro-6,7-dihydro-5H-thieno[3,2-b]pyran-6-yl)-N-[(2,4-dimethoxyphenyl)methyl]carbamate (4) (40 mg, 90.92 μmol, 55.00% yield) as yellow oil. ¹H NMR (400 MHz, CHLOROFORM-d) δ=7.12 (br s, 1H), 6.84 (s, 1H), 6.51-6.40 (m, 2H), 4.54-4.04 (m, 5H), 3.80 (d, J=11.4 Hz, 6H), 3.11 (br s, 1H), 2.72 (br d, J=13.8 Hz, 1H), 1.48 (br s, 9H).

### Procedure for preparation of 3-chloro-6,7-dihydro-5H-thieno[3,2-b]pyran-6-amine hydrochloride (215)

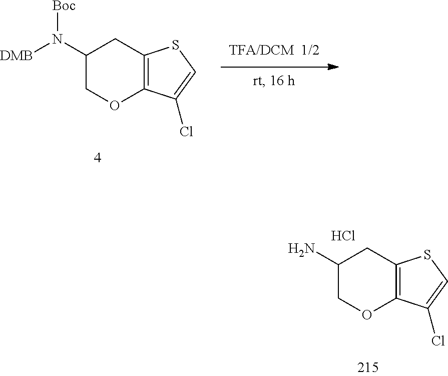

To a solution of tert-butyl N-(3-chloro-6,7-dihydro-5H-thieno[3,2-b]pyran-6-yl)-N-[(2,4-dimethoxyphenyl)methyl]carbamate (4) (40 mg, 90.92 μmol, 1 eq) in DCM (1 mL) was added TFA (767.50 mg, 6.73 mmol, 0.5 mL, 74.04 eq). The mixture was stirred at 25° C. for 16 hr. The reaction mixture was concentrated under reduced pressure to remove TFA. Sat. aq. NaHCO₃ solution was added into the mixture to adjust pH to 6. Methanol was added (2 mL). The mixture was filtered. The filtrate was purified by prep-HPLC (column: Phenomenex luna C18 150*25 mm*10 μm; mobile phase: [water(HCl)-ACN]; gradient:51%-20% B over 10 min) to afford 3-chloro-6,7-dihydro-5H-thieno[3,2-b]pyran-6-amine (5) (10 mg, 52.73 μmol, 57.99% yield) as a white solid. ¹H NMR (400 MHz, METHANOL-d4) δ=7.21 (s, 1H), 4.44-4.37 (m, 1H), 4.25 (d, J=11.9 Hz, 1H), 3.95 (br s, 1H), 3.34 (br d, J=5.6 Hz, 1H), 2.88 (br d, J=17.0 Hz, 1H). LC-MS: m/z [M+H]⁺=190.0

### Synthetic scheme for preparation of 2-chloro-4,5,6,7-tetrahydrobenzothiophen-5-amine hydrochloride (216)

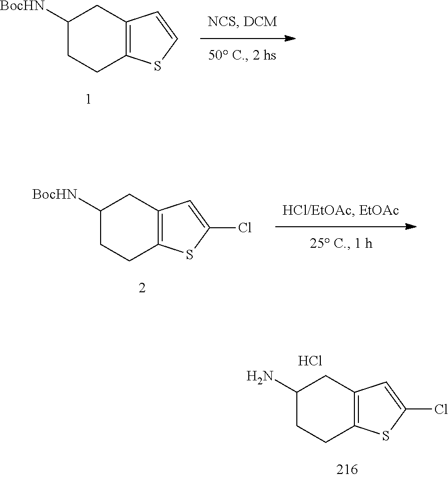 [SMILES: CC(C)(C)OC(=O)NC1CCc2sccc2C1 ; CC(C)(C)OC(=O)NC1CCc2sc(Cl)cc2C1 ; Cl ; NC1CCc2sc(Cl)cc2C1]

### Procedure for preparation of tert-butyl N-(2-chloro-4,5,6,7-tetrahydrobenzothiophen-5-yl)carbamate (2)

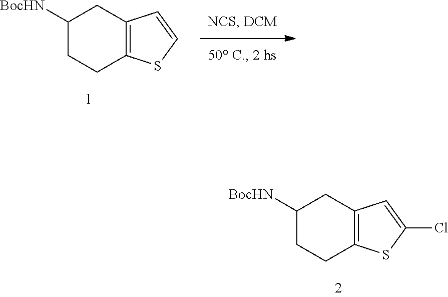 [SMILES: CC(C)(C)OC(=O)NC1CCc2sccc2C1 ; CC(C)(C)OC(=O)NC1CCc2sc(Cl)cc2C1]

To a solution of tert-butyl N-(4,5,6,7-tetrahydrobenzothiophen-5-yl)carbamate (1) (300 mg, 1.18 mmol, 1 eq) in DMF (2 mL) was added NCS (197.64 mg, 1.48 mmol, 1.25 eq). The mixture was stirred at 50° C. for 2 hr. The reaction mixture was poured into H₂O (20 mL). The reaction was diluted with aq. sat. NaHCO₃ (20 mL) and extracted with EtOAc mL (10 mL*3). The combined organic layers were washed with brine (10 mL*3), dried over anhydrous sodium sulfate, filtered and concentrated under reduced pressure to give a residue. The residue was purified by prep-HPLC (column: Phenomenex luna C18 150*25 mm*10 μm; mobile phase: [water(HCl)-ACN]; gradient:50%-80% B over 10 min). Compound tert-butyl N-(2-chloro-4,5,6,7-tetrahydrobenzothiophen-5-yl)carbamate (2) (80 mg, 272.41 μmol, 23.01% yield, 98% purity) was obtained as a white solid. LC-MS: m/z [M+H-tBu]⁺=232.1.

### Procedure for preparation of 2-chloro-4,5,6,7-tetrahydrobenzothiophen-5-amine hydrochloride (216)

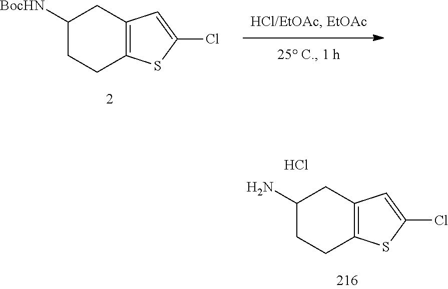 [SMILES: CC(C)(C)OC(=O)NC1CCc2sc(Cl)cc2C1 ; Cl ; NC1CCc2sc(Cl)cc2C1]

To a solution of tert-butyl N-(2-chloro-4,5,6,7-tetrahydrobenzothiophen-5-yl)carbamate (2) (80 mg, 277.97 μmol, 1 eq) in EtOAc (1 mL) was added HCl/EtOAc (4 M, 2 mL, 28.78 eq). The mixture was stirred at 25° C. for 1 hr. The reaction mixture was concentrated under vacuum to give a residue. The residue was washed with EtOAc (2 mL) and the filter cake was dried to give compound 2-chloro-4,5,6,7-tetrahydrobenzothiophen-5-amine hydrochloride (216) (50.61 mg, 266.90 μmol, 96.02% yield) as a white solid. ¹H NMR (400 MHz, METHANOL-d₄) δ=6.69 (s, 1H), 3.66-3.51 (m, 1H), 3.09-2.96 (m, 1H), 2.95-2.79 (m, 2H), 2.67-2.53 (m, 1H), 2.31-2.17 (m, 1H), 1.94 (dddd, J=7.0, 8.8, 10.8, 12.8 Hz, 1H). LC-MS: m/z [M+H]⁺=188.2.

### Synthetic scheme for preparation of 3-chloro-4,5,6,7-tetrahydrobenzothiophen-6-amine hydrochloride (217)

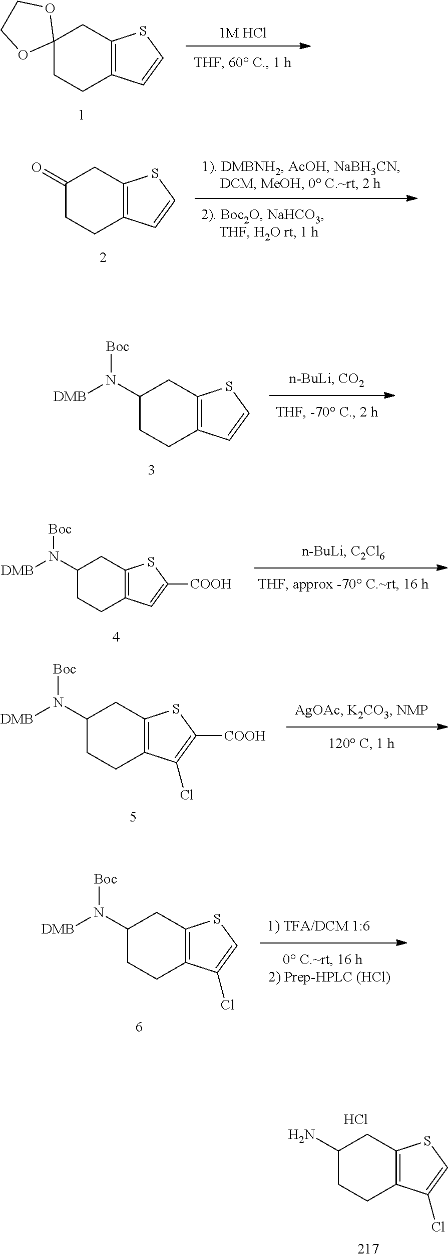

### Procedure for preparation of 5,7-dihydro-4H-benzothiophen-6-one (2)

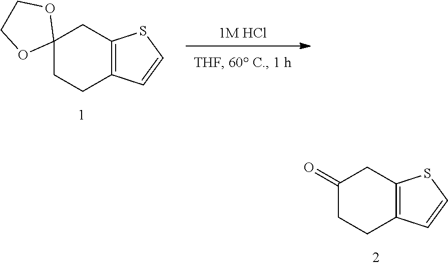

The mixture of spiro[1,3-dioxolane-2,6′-5,7-dihydro-4H-benzothiophene](1) (8 g, 40.7 mmol, 1.00 eq) in THF (40 mL), HCl (1 M, 80 mL, 1.96 eq) and acetone (4.73 g, 81.5 mmol, 6.0 mL, 2.00 eq) was stirred at 60° C. for 1 h. The reaction mixture was separated and the aqueous phase was extracted with EtOAc (30 mL*3). The combined organic layers were washed with sat. NaHCO₃ (30 mL), brine (30 mL), dried over anhydrous sodium sulfate, filtered and concentrated under vacuum to give 5,7-dihydro-4H-benzothiophen-6-one (2) (5.8 g, 38.1 mmol, 93.5% yield) as a purple oil which was used into next step without further purification. ¹H NMR (400 MHz, CDCl₃-d) 6=7.18 (d, J=5.2 Hz, 1H), 6.85 (d, J=5.2 Hz, 1H), 3.64 (s, 2H), 3.03 (t, J=6.8 Hz, 2H), 2.69 (t, J=6.8 Hz, 2H). LCMS: m/z [M+H]⁺=152.9.

### Procedure for preparation of tert-butyl N-[(2,4-dimethoxyphenyl)methyl]-N-(4,5,6,7-tetrahydrobenzothiophen-6-yl)carbamate (3)

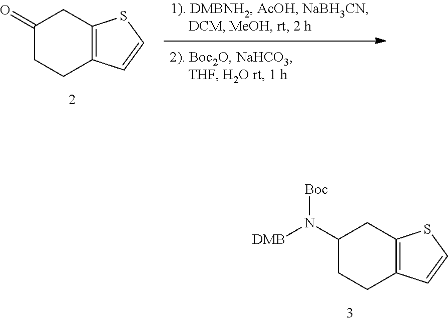

To the mixture of 5,7-dihydro-4H-benzothiophen-6-one (2) (5.8 g, 38.1 mmol, 1.00 eq), (2,4-dimethoxyphenyl)methanamine (7.65 g, 45.7 mmol, 1.20 eq) and AcOH (114 mg, 1.91 mmol, 0.05 eq) in DCM (50 mL) and MeOH (10 mL) was stirred at 15-20° C. for 0.5 h. Then NaBH₃CN (3.59 g, 57.2 mmol, 1.50 eq) was added and the resulting mixture was stirred at 15-20° C. for 2 h. Then to the mixture was added H₂O (30 mL), aq. saturated NaHCO₃ (9.60 g, 114 mmol, 4.45 mL, 3.00 eq) and Boc₂O (16.6 g, 76.2 mmol, 2.00 eq). The resulting mixture was stirred at 15-20° C. for 1 h. The reaction mixture was concentrated under vacuum to remove organic solvents. The residue was diluted with water (50 mL) and extracted with EtOAc (40 mL*3). The combined organic phase was washed with brine (50 mL), dried over anhydrous sodium sulfate, filtered and concentrated under vacuum to give a residue which was purified by flash silica gel chromatography (ISCO®; 80 g SepaFlash® Silica Flash Column, eluent 0~15% Ethyl acetate/Petroleum ether gradient @60 mL/min) to give tert-butyl N-[(2,4-dimethoxyphenyl)methyl]-N-(4,5,6,7-tetrahydrobenzothiophen-6-yl)carbamate (3) (10.2 g, 25.3 mmol, 66.3% yield) as a yellow gum. ¹H NMR (400 MHz, CDCl₃-d) 6=7.14 (br d, J=8.1 Hz, 1H), 7.04 (br d, J=4.6 Hz, 1H), 6.71 (d, J=4.9 Hz, 1H), 6.48 (dd, J=2.4, 8.4 Hz, 1H), 6.43 (d, J=2.3 Hz, 1H), 4.63-4.22 (m, 2H), 3.80 (d, J=11.3 Hz, 6H), 3.05-2.79 (m, 3H), 2.77-2.56 (m, 2H), 1.89 (br d, J=1.5 Hz, 2H), 1.55-1.33 (m, 9H) LCMS: m/z [M+Na]⁺=426.2.

### Procedure for preparation of 6-[tert-butoxycarbonyl-[(2,4-dimethoxyphenyl)methyl]amino]-4,5,6,7-tetrahydrobenzothiophene-2-carboxylic acid (4)

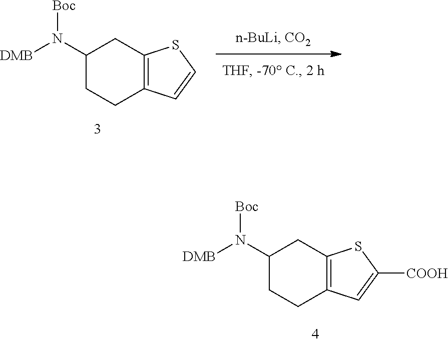

To solution of tert-butyl N-[(2,4-dimethoxyphenyl)methyl]-N-(4,5,6,7-tetrahydrobenzothiophen-6-yl)carbamate (3) (9.7 g, 24.0 mmol, 1.00 eq) in THF (150 mL) was added n-BuLi (2.5 M, 1.50 eq) at −70° C. The mixture was stirred at −70° C. for 0.5 h. Then CO₂ (10.6 g, 240 mmol, 10.0 eq) (dried via H₂SO₄) was bubbled into the reaction mixture. Then the mixture was stirred at −70° C. for 1.5 h. The reaction mixture was quenched with aq. sat. NH₄Cl (75 mL), diluted with water (75 mL) and the aqueous layer pH was adjusted to 3-4 with citric acid and extracted with EtOAc (50 mL*3). The combined organic phases were washed with brine (100 mL), dried over anhydrous sodium sulfate, filtered and concentrated under vacuum to give a residue which was purified by flash silica gel chromatography (ISCO®; 120 g SepaFlash® Silica Flash Column, eluent 0~30% Ethyl acetate/Petroleum ether gradient @70 mL/min) to give 6-[tert-butoxycarbonyl-[(2,4-dimethoxyphenyl)methyl]amino]-4,5,6,7-tetrahydrobenzothiophene-2-carboxylic acid (4) (5.9 g, 13.2 mmol, 54.8% yield) as a yellow solid. LCMS: m/z [M+Na]⁺=470.2.

### Procedure for preparation of 6-[tert-butoxycarbonyl-[(2,4-dimethoxyphenyl)methyl]amino]-3-chloro-4,5,6,7-tetrahydrobenzothiophene-2-carboxylic acid (5)

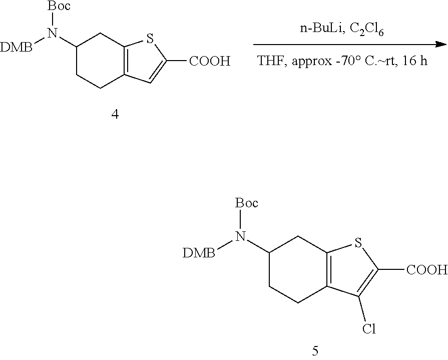

To the solution of 6-[tert-butoxycarbonyl-[(2,4-dimethoxyphenyl)methyl]amino]-4,5,6,7-tetrahydrobenzothiophene-2-carboxylic acid (4) (1.00 g, 2.23 mmol, 1.00 eq) in THF (10 mL) was added n-BuLi (2.5 M, 2.50 eq) at−70~−65° C. under N₂ atmosphere. After stirring at −70° C. for 0.5 h, a solution of 1,1,1,2,2,2-hexachloroethane (582 mg, 2.46 mmol, 1.10 eq) in THF (2 mL) was added drop-wise. Then the resulting mixture was allowed to warm to room temperature slowly and stirred for 16 h. The reaction mixture was quenched by aq. sat. NH₄Cl (20 mL), diluted with water (20 mL), and the aq layer pH was adjusted pH to 3-4 with sat. aq. citric acid and extracted with EtOAc (20 mL*3). The combined organic phases were washed with brine (30 mL), dried over anhydrous sodium sulfate, filtered and concentrated under vacuum to give a residue that was a mixture of desired material 6-[tert-butoxycarbonyl-[(2,4-dimethoxyphenyl)methyl]amino]-3-chloro-4,5,6,7-tetrahydrobenzothiophene-2-carboxylic acid (5) and 60% of starting material (4) that was isolated as as a yellow gum (1.5 g, crude) which was used into next step without further purification.

### Procedure for preparation of tert-butyl N-(3-chloro-4,5,6,7-tetrahydrobenzothiophen-6-yl)-N-[(2,4-dimethoxyphenyl)methyl]carbamate (6)

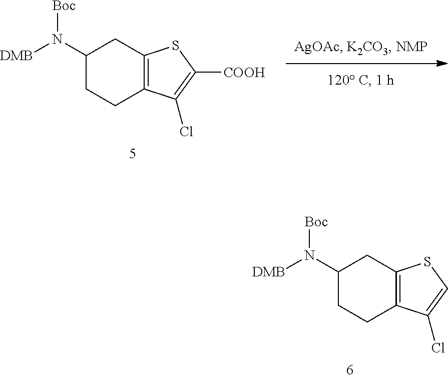

To the mixture of 6-[tert-butoxycarbonyl-[(2,4-dimethoxyphenyl)methyl]amino]-3-chloro-4,5,6,7-tetrahydrobenzothiophene-2-carboxylic acid (5) (1.3 g, 2.70 mmol, 1.00 eq) in NMP (13 mL) was added AgOAc (135 mg, 809 μmol, 0.30 eq) and K₂CO₃ (1.12 g, 8.09 mmol, 3.00 eq). The resulting mixture was stirred at 120° C. for 1 h. The reaction mixture was diluted with EtOAc (20 mL) and filtered. The filtrate was washed with water (60 mL). The aqueous phase was extracted with EtOAc (15 mL*3). The combined organic phases were washed with brine (30 mL), dried over anhydrous sodium sulfate, filtered and concentrated under vacuum to give a residue which was purified by flash silica gel chromatography (ISCO®; 12 g SepaFlash® Silica Flash Column, eluent 0~15% Ethyl acetate/Petroleum ether gradient @40 mL/min) to give tert-butyl N-(3-chloro-4,5,6,7-tetrahydrobenzothiophen-6-yl)-N-[(2,4-dimethoxyphenyl)methyl]carbamate (6) (260 mg, 594 μmol, 22.0% yield) as a colorless gum. ¹H NMR (400 MHz, CDCl₃-d) 6=7.14 (br d, J=7.0 Hz, 1H), 6.89 (s, 1H), 6.48 (dd, J=2.4, 8.4 Hz, 1H), 6.44 (d, J=2.1 Hz, 1H), 4.52-4.26 (m, 2H), 3.81 (d, J=9.8 Hz, 6H), 2.95-2.77 (m, 2H), 2.76-2.59 (m, 2H), 2.56-2.39 (m, 1H), 1.92-1.81 (m, 2H), 1.53-1.36 (m, 9H).

### Procedure for preparation of 3-chloro-4,5,6,7-tetrahydrobenzothiophen-6-amine hydrochloride (217)

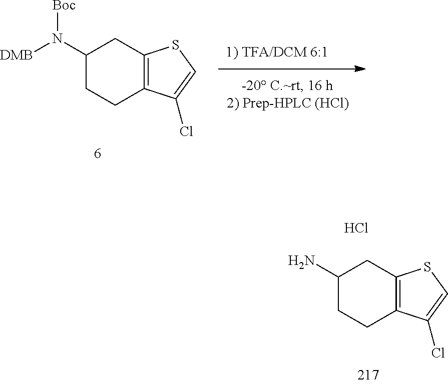

To the mixture of tert-butyl N-(3-chloro-4,5,6,7-tetrahydrobenzothiophen-6-yl)-N-[(2,4-dimethoxyphenyl)methyl]carbamate (6) (240 mg, 548 μmol, 1.00 eq) in DCM (6 mL) was added TFA (1.54 g, 13.46 mmol, 1 mL, 24.57 eq) at −20° C. Then the mixture was warmed to 10-15° C. slowly and stirred at 15-20° C. for 16 h. The reaction mixture was poured into aq. sat. NaHCO₃ (40 mL) and extracted with EtOAc (15 mL*3). The combined organic phases were dried over anhydrous sodium sulfate, filtered and concentrated under vacuum to give a residue which was purified by prep-HPLC (column: Phenomenex luna C18 150*25 mm*10 μm; mobile phase: [water(HCl)-ACN]; gradient: 1%-30% B over 10 min) to give 3-chloro-4,5,6,7-tetrahydrobenzothiophen-6-amine hydrochloride (217) (43.39 mg, 194 μmol, 35.3% yield) as a white solid. ¹H NMR (400 MHz, MeOD-d₄) δ=7.19 (s, 1H), 3.70-3.59 (m, 1H), 3.22 (dd, J=5.2, 16.1 Hz, 1H), 2.90-2.72 (m, 2H), 2.71-2.59 (m, 1H), 2.29-2.18 (m, 1H), 1.99-1.87 (m, 1H). LCMS: m/z [M+H]⁺=188.0.

### Synthetic scheme for preparation of 3-fluoro-6-methyl-5,7-dihydro-4H-benzothiophen-6-amine hydrochloride (218)

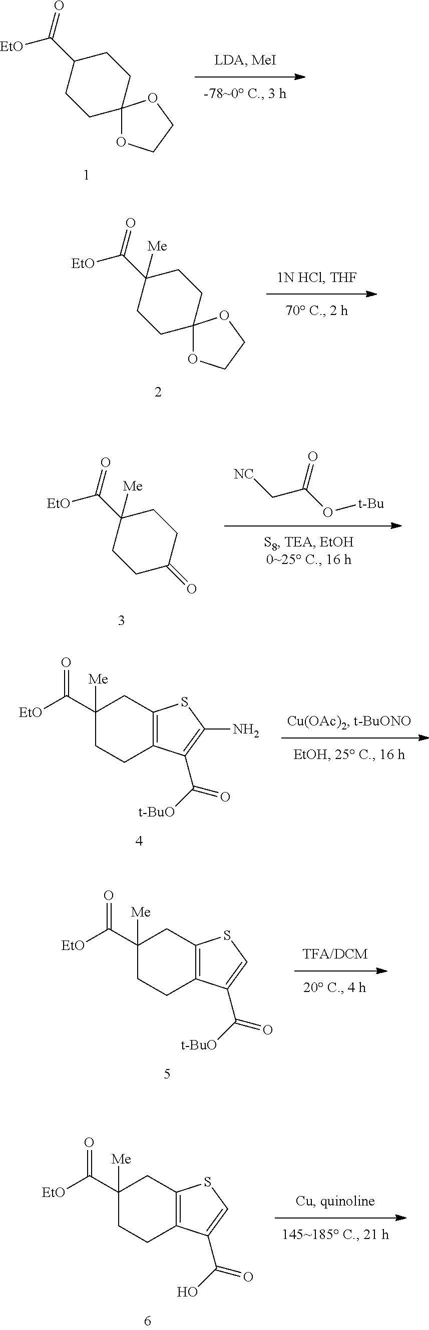

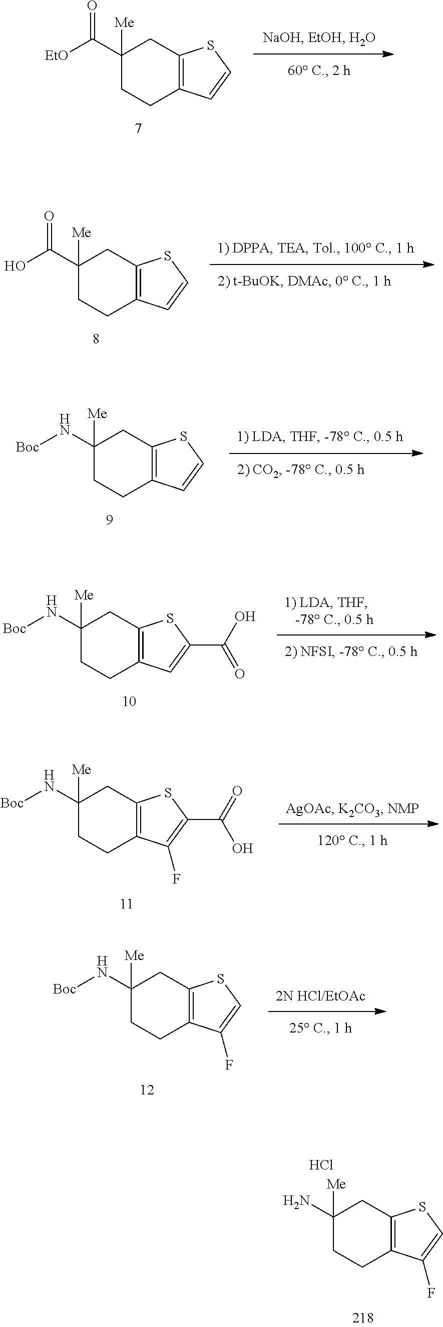

### Procedure for preparation of ethyl 8-methyl-1,4-dioxaspiro[4.5]decane-8-carboxylate (2)

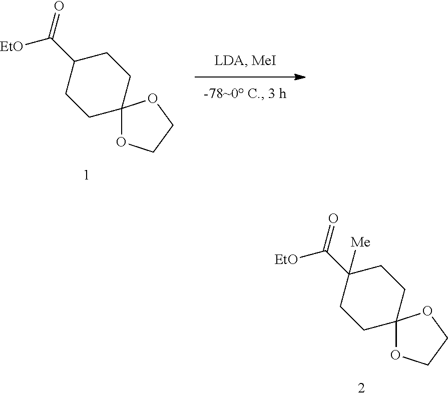 [SMILES: CCOC(=O)C1CCC2(CC1)OCCO2 ; CCOC(=O)C1(C)CCC2(CC1)OCCO2]

To a solution of LDA (2 M, 280.04 mL, 1.2 eq) in THF (600 mL) was added a solution of ethyl 1,4-dioxaspiro[4.5]decane-8-carboxylate (1) (100 g, 466.73 mmol, 1 eq) in THF (50 mL) dropwise at −78° C. under N₂. After 1 h to the reaction was added a solution of MeI (86.12 g, 606.75 mmol, 37.77 mL, 1.3 eq) in THF (50 mL) dropwise. The mixture was stirred at −78° C. for 1 h, and then was warmed to 0° C. and stirred at this temperature for 1 hr. The reaction mixture was then concentrated under reduced pressure to remove solvent. The reaction mixture was quenched by addition sat. aq. NH₄Cl 200 mL at 0° C., and then diluted with H₂O (200 mL) and extracted with EtOAc (300 mL*3). The combined organic layers were washed with brine (200 mL), dried over anhydrous Na₂SO₄, filtered and concentrated under reduced pressure to give ethyl 8-methyl-1,4-dioxaspiro[4.5]decane-8-carboxylate (2) (101 g, 442.43 mmol, 94.79% yield) as a yellow oil. ¹H NMR. ¹H NMR (400 MHz, CHLOROFORM-d) δ=4.14 (q, J=7.2 Hz, 2H), 3.92 (s, 4H), 2.18-2.08 (m, 2H), 1.69-1.55 (m, 4H), 1.55-1.45 (m, 2H), 1.24 (t, J=7.2 Hz, 3H), 1.18 (s, 3H).

### Procedure for preparation of ethyl 1-methyl-4-oxo-cyclohexanecarboxylate (3)

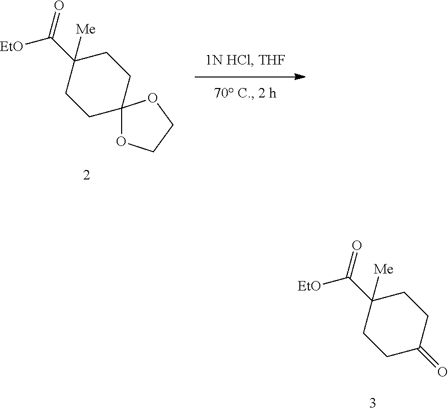 [SMILES: CCOC(=O)C1(C)CCC2(CC1)OCCO2 ; CCOC(=O)C1(C)CCC(=O)CC1]

To a solution of ethyl 8-methyl-1,4-dioxaspiro[4.5]decane-8-carboxylate (2) (65 g, 284.73 mmol, 1 eq) in THF (650 mL) was added aq. HCl (1 M, 650.00 mL, 2.28 eq), the mixture was stirred at 70° C. for 2 hours. The reaction was cooled to r.t., the pH of the mixture was adjusted to 7-8 by adding sat. aq. NaHCO₃, and then the mixture was extracted with EtOAc (200 mL*2). The combined organic layer and washed with brine (200 mL), dried over anhydrous Na₂SO₄, filtered and concentrated to afford ethyl 1-methyl-4-oxo-cyclohexanecarboxylate (3) (52 g, crude) as yellow oil.

### Procedure for preparation of 3-tert-butyl 6-ethyl 2-amino-6-methyl-5,7-dihydro-4H-benzothiophene-3,6-dicarboxylate (4)

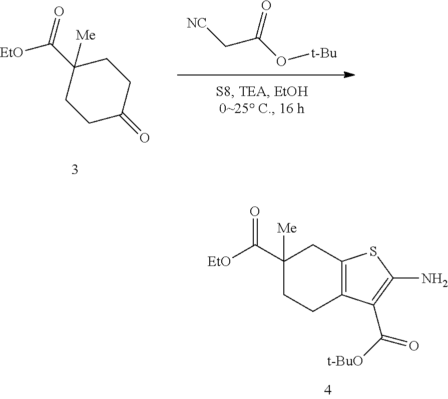 [SMILES: CCOC(=O)C1(C)CCC(=O)CC1 ; CC(C)(C)OC(=O)CC#N ; CCOC(=O)C1(C)CCc2c(sc(N)c2C(=O)OC(C)(C)C)C1]

To a solution of ethyl 1-methyl-4-oxo-cyclohexanecarboxylate (3) (52 g, 282.25 mmol, 1 eq) in EtOH (500 mL) was added S₈ (15.02 g, 468.54 mmol, 1.66 eq) at 0° C., and then tert-butyl 2-cyanoacetate (47.81 g, 338.70 mmol, 48.44 mL, 1.2 eq). The resulting suspension was treated with TEA (57.12 g, 564.51 mmol, 78.57 mL, 2 eq) (maintaining internal temperature below 10° C.) and then the reaction was stirred at 25° C. for 16 hours. The reaction was then filtered, the filtrate was concentrated to remove most of the EtOH, and then diluted with EtOAc (600 mL), washed with sat. aq. citric acid (250 mL), sat. aq. NaHCO₃ (250 mL) and brine (200 mL) and then dried over anhydrous Na₂SO₄, filtered and concentrated to afford 3-tert-butyl 6-ethyl 2-amino-6-methyl-5,7-dihydro-4H-benzothiophene-3,6-dicarboxylate (4) (100 g, crude) as yellow oil. LC-MS: m/z [M+H]⁺=340.2.

### Procedure for preparation of 3-tert-butyl 6-ethyl 6-methyl-5,7-dihydro-4H-benzothiophene-3,6-dicarboxylate (5)

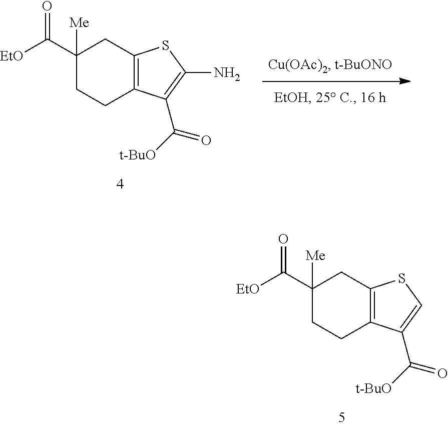 [SMILES: CCOC(=O)C1(C)CCc2c(sc(N)c2C(=O)OC(C)(C)C)C1 ; CCOC(=O)C1(C)CCc2c(C(=O)OC(C)(C)C)csc2C1]

To a solution of Cu(OAc)₂ (96.32 g, 530.27 mmol, 2 eq) and t-BuONO (27.34 g, 265.14 mmol, 31.53 mL, 1 eq) in EtOH (2000 mL) was added 3-tert-butyl 6-ethyl 2-amino-6-methyl-5,7-dihydro-4H-benzothiophene-3,6-dicarboxylate (4) (90 g, 265.14 mmol, 1 eq) at 25° C. The reaction mixture was allowed to stir at 25° C. for 16 h. The reaction was then filtered and the filtrate was concentrated to remove most of the EtOH and then diluted with EtOAc (800 mL). The resulting mixture was washed with water (500 mL*2), brine (300 mL), dried over anhydrous Na₂SO₄, filtered and concentrated to get a residue. The residue was purified by silica gel column (PE:EtOAc=50:1 to 10:1) to afford 3-tert-butyl 6-ethyl 6-methyl-5,7-dihydro-4H-benzothiophene-3,6-dicarboxylate (5) (23 g, 70.89 mmol, 26.74% yield) as yellow oil.

### Procedure for preparation of 6-ethoxycarbonyl-6-methyl-5,7-dihydro-4H-benzothiophene-3-carboxylic acid (6)

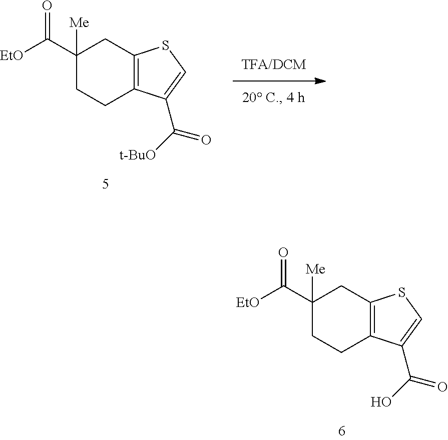 [SMILES: CCOC(=O)C1(C)CCc2c(C(=O)OC(C)(C)C)csc2C1 ; CCOC(=O)C1(C)CCc2c(C(=O)O)csc2C1]

To a solution of 3-tert-butyl 6-ethyl 6-methyl-5,7-dihydro-4H-benzothiophene-3,6-dicarboxylate (5) (23 g, 70.89 mmol, 1 eq) in DCM (200 mL) was added TFA (61.40 g, 538.49 mmol, 40 mL, 7.60 eq), the mixture was stirred at 20° C. for 4 hours. The reaction pH was adjusted to 4-5 with sat. aq. NaHCO₃ and the mixture was then extracted with DCM (200 mL*2). The organic layers were combined and washed with brine (200 mL), dried over anhydrous Na₂SO₄, filtered and concentrated to afford 6-ethoxycarbonyl-6-methyl-5,7-dihydro-4H-benzothiophene-3-carboxylic acid (6) (16 g, 59.63 mmol, 84.11% yield) as yellow solid. LC-MS: m/z [M−H]⁻=267.1.

### Procedure for preparation of ethyl 6-methyl-5,7-dihydro-4H-benzothiophene-6-carboxylate (7)

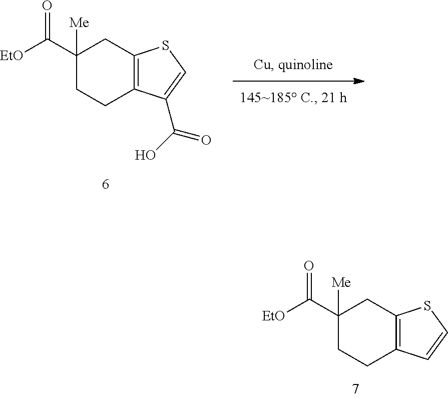 [SMILES: CCOC(=O)C1(C)CCc2c(C(=O)O)csc2C1 ; CCOC(=O)C1(C)CCc2ccsc2C1]

To a solution of 6-ethoxycarbonyl-6-methyl-5,7-dihydro-4H-benzothiophene-3-carboxylic acid (6) (6 g, 22.36 mmol, 1 eq) in quinoline (60 mL) was added Cu (4.26 g, 67.08 mmol, 3 eq) and the mixture was stirred at 140° C. for 16 hours and 185° C. for 5 hours. The reaction was cooled to r.t., filtered and diluted with EtOAc (100 mL). The mixture waswashed with 2 N aq.HCl (100 mL), brine (100 mL), dried over anhydrous Na₂SO₄ filtered and concentrated to get a residue. The residue was purified by silica gel column (PE:EtOAc=100:1 to 20:1) to afford ethyl 6-methyl-5,7-dihydro-4H-benzothiophene-6-carboxylate (7) (4 g, 17.83 mmol, 79.75% yield) as light yellow oil. ¹H NMR (400 MHz, CHLOROFORM-d) δ=7.06 (d, J=5.2 Hz, 1H), 6.74 (d, J=5.2 Hz, 1H), 4.13 (q, J=7.2 Hz, 2H), 3.29 (d, J=16.0 Hz, 1H), 2.76-2.61 (m, 3H), 2.21-2.10 (m, 1H), 1.86-1.74 (m, 1H), 1.31 (s, 3H), 1.22 (t, J=7.2 Hz, 3H).

### Procedure for preparation of 6-methyl-5,7-dihydro-4H-benzothiophene-6-carboxylic acid (8)

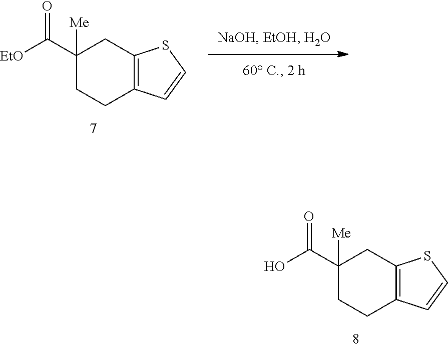 [SMILES: CCOC(=O)C1(C)CCc2ccsc2C1 ; CC1(C(=O)O)CCc2ccsc2C1]

To a solution of ethyl 6-methyl-5,7-dihydro-4H-benzothiophene-6-carboxylate (7) (3.5 g, 15.60 mmol, 1 eq) in EtOH (20 mL) was added aq. NaOH (3 M, 14.56 mL, 2.8 eq) and the mixture was stirred at 60° C. for 2 hours. The reaction mixture was then concentrated to get a residue, diluted with EtOAc (30 mL), and acidified to pH 4-5 by addition of aq. 1 N HCl. This was followed by extraction with EtOAc (30 mL). The EtOAc layer was then washed with brine (30 mL), dried over anhydrous Na₂SO₄, filtered and concentrated to afford 6-methyl-5,7-dihydro-4H-benzothiophene-6-carboxylic acid (8) (2.7 g, 13.76 mmol, 88.17% yield) as yellow solid.

### Procedure for preparation of tert-butyl N-(6-methyl-5,7-dihydro-4H-benzothiophen-6-yl)carbamate (9)

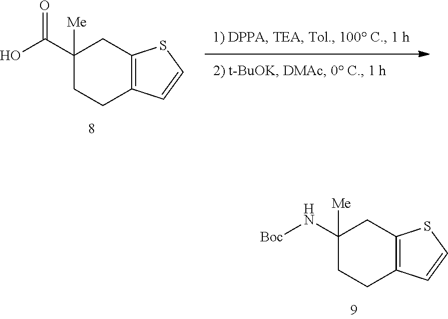 [SMILES: CC1(C(=O)O)CCc2ccsc2C1 ; CC1(NC(=O)OC(C)(C)C)CCc2ccsc2C1]

To a solution of 6-methyl-5,7-dihydro-4H-benzothiophene-6-carboxylic acid (8) (2.7 g, 13.76 mmol, 1 eq) in Tol. (25 mL) was added DPPA (3.79 g, 13.76 mmol, 2.97 mL, 1 eq) and TEA (1.53 g, 15.13 mmol, 2.11 mL, 1.1 eq). The mixture was stirred at 100° C. for 1 hour, then cooled to 20° C. and washed with H₂O (20 mL). The organic layer was then collected, dried over Na₂SO₄, filtered and concentrated to give a residue, which was dissolved in DMAc (25 mL) and cooled to 0° C. To this mixture was then added t-BuOK (1.70 g, 15.13 mmol, 1.1 eq). The reaction was stirred at 0~15° C. for 1 hour and then quenched with sat. aq. NH₄Cl (30 mL). The mixture was extracted with EtOAc (30 mL), the organic layer was washed with brine (20 mL), dried over anhydrous Na₂SO₄, filtered and concentrated. The residue was purified by silica gel column (PE:EtOAc=50:1 to 10:1) to afford tert-butyl N-(6-methyl-5,7-dihydro-4H-benzothiophen-6-yl)carbamate (9) (0.7 g, 2.62 mmol, 19.03% yield) as light yellow oil. ¹H NMR (400 MHz, CHLOROFORM-d) δ=7.04 (d, J=5.0 Hz, 1H), 6.73 (d, J=5.1 Hz, 1H), 4.46 (br s, 1H), 2.93-2.80 (m, 2H), 2.66-2.53 (m, 2H), 2.43-2.27 (m, 1H), 1.61 (ddd, J=6.3, 8.3, 13.3 Hz, 1H), 1.43 (s, 3H), 1.39 (s, 9H). LC-MS: m/z [M+H]⁺=151.0. LC-MS: m/z [M+H-tBu]⁺=212.1.

### Procedure for preparation of 6-(tert-butoxycarbonylamino)-6-methyl-5,7-dihydro-4H-benzothiophene-2-carboxylic acid (10)

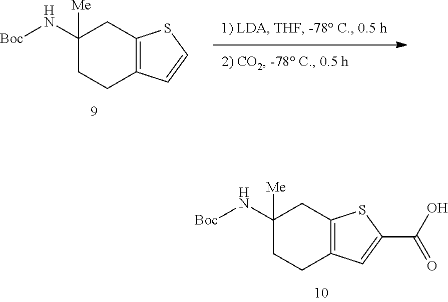 [SMILES: CC1(NC(=O)OC(C)(C)C)CCc2ccsc2C1 ; CC1(NC(=O)OC(C)(C)C)CCc2cc(C(=O)O)sc2C1]

To a solution of tert-butyl N-(6-methyl-5,7-dihydro-4H-benzothiophen-6-yl)carbamate (9) (2.26 g, 8.45 mmol, 1 eq) in THF (15 mL) was added LDA (2 M, 9.30 mL, 2.20 eq) at −78° C. The mixture was stirred for 0.5 hour, then CO₂ (balloon, 15 psi) was bubbled. The reaction was stirred at −78° C. for an additional 0.5 hour and then quenched with aq. sat. aq. NH₄Cl (50 mL). The mixture was acidified to pH 4-5 with 1N HCl aq. and extracted with EtOAc (50 mL*2). The combined organic layers were washed with brine (20 mL), dried over anhydrous Na₂SO₄, filtered and concentrated. The residue was purified by silica gel column (PE:EtOAc=50:1 to 3:1) to afford 6-(tert-butoxycarbonylamino)-6-methyl-5,7-dihydro-4H-benzothiophene-2-carboxylic acid (10) (0.8 g, crude) as yellow oil. LC-MS: m/z [M+H-tBu]⁺=256.2.

### Procedure for preparation of 6-(tert-butoxycarbonylamino)-3-fluoro-6-methyl-5,7-dihydro-4H-benzothiophene-2-carboxylic acid (11)

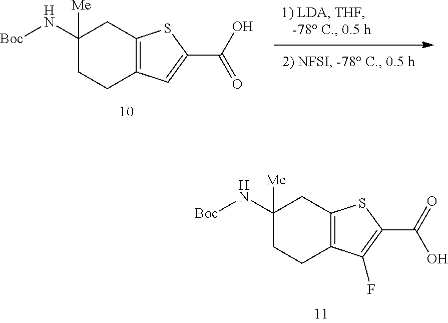 [SMILES: CC1(NC(=O)OC(C)(C)C)CCc2cc(C(=O)O)sc2C1 ; CC1(NC(=O)OC(C)(C)C)CCc2c(sc(C(=O)O)c2F)C1]

To a solution of 6-(tert-butoxycarbonylamino)-6-methyl-5,7-dihydro-4H-benzothiophene-2-carboxylic acid (10) (800.00 mg, 2.57 mmol, 1 eq) in THF (8 mL) was added n-BuLi (2.5 M, 3.39 mL, 3.3 eq) at −78° C. The mixture was stirred for 0.5 hour, and then NFSI (2.03 g, 6.42 mmol, 2.5 eq) in THF (5 mL) was added. After stirring at −78° C. for an additional 0.5 hour the reaction was quenched with sat. aq. NH₄Cl (30 mL) and acidified to pH 4-5 with aq. 1N HCl. The mixture was then extracted with EtOAc (30 mL*2). The combined organic layers were washed with brine (30 mL), dried over anhydrous Na₂SO₄, filtered and concentrated to afford 6-(tert-butoxycarbonylamino)-3-fluoro-6-methyl-5,7-dihydro-4H-benzothiophene-2-carboxylic acid (11) (0.8 g, crude) as a yellow oil. LC-MS: m/z [M+H-tBu]⁺=274.1.

### Procedure for preparation of tert-butyl N-(3-fluoro-6-methyl-5,7-dihydro-4H-benzothiophen-6-yl)carbamate (12)

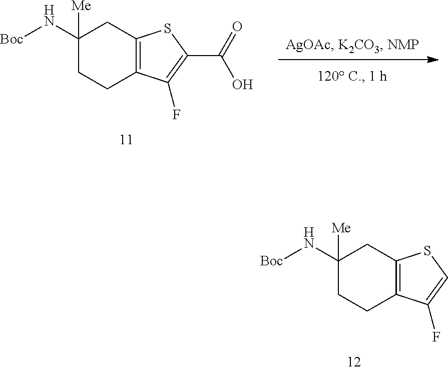 [SMILES: CC1(NC(=O)OC(C)(C)C)CCc2c(sc(C(=O)O)c2F)C1 ; CC1(NC(=O)OC(C)(C)C)CCc2c(F)csc2C1]

To a solution of 6-(tert-butoxycarbonylamino)-3-fluoro-6-methyl-5,7-dihydro-4H-benzothiophene-2-carboxylic acid (11) (0.8 g, 2.43 mmol, 1 eq) in NMP (10 mL) was added AgOAc (121.62 mg, 728.63 μmol, 0.3 eq) and K₂CO₃ (1.01 g, 7.29 mmol, 3 eq). The mixture was stirred at 120° C. for 1 hour, cooled to rt, quenched with water (20 mL), diluted with EtOAc (30 mL) and filtered. The filtrate was extracted with EtOAc (15 mL*2). The organic layers were combined and washed with brine (20 mL), dried over anhydrous Na₂SO₄, filtered and concentrated. The residue was purified by Prep-TLC (PE:EtOAc=10:1) and by Prep-HPLC (column: Phenomenex Luna C18 150*25 mm*10 μm; mobile phase: [water(TFA)-ACN]; gradient:56%-86% B over 9 min) to afford tert-butyl N-(3-fluoro-6-methyl-5,7-dihydro-4H-benzothiophen-6-yl)carbamate (12) (5 mg, 17.52 μmol, 25.00% yield) as light yellow solid. LC-MS: m/z [M+H-tBu]⁺=230.1.

### Procedure for preparation of 3-fluoro-6-methyl-5,7-dihydro-4H-benzothiophen-6-amine hydrochloride (218)

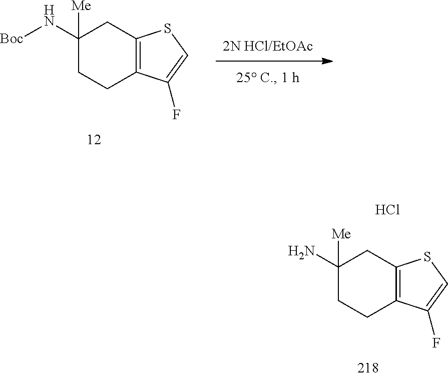 [SMILES: CC1(NC(=O)OC(C)(C)C)CCc2c(F)csc2C1 ; Cl ; CC1(N)CCc2c(F)csc2C1]

To a solution of tert-butyl N-(3-fluoro-6-methyl-5,7-dihydro-4H-benzothiophen-6-yl)carbamate (12) (5 mg, 17.52 μmol, 1 eq) in EtOAc (2 mL) was added HCl/EtOAc (4 M, 2 mL, 456.60 eq), the mixture was stirred at 20° C. for 1 hour. The reaction was concentrated to get a residue that was lyophilized to afford a light yellow solid, which was purified by Prep-HPLC (column: Phenomenex luna C18 150*25 mm*10 μm; mobile phase: [water(HCl)-ACN]; gradient:1%-25% B over 10 min) to afford 3-fluoro-6-methyl-5,7-dihydro-4H-benzothiophen-6-amine hydrochloride (218) (3.17 mg, 14.27 μmol, 81.46% yield) as white solid. LC-MS: m/z [M+H]⁺=186.0. ¹H NMR (400 MHz, METHANOL-d₄) δ=6.76 (s, 1H), 2.95 (s, 2H), 2.75-2.60 (m, 2H), 2.07-1.92 (m, 2H), 1.44 (s, 3H).

### Synthetic scheme for preparation of 2,3-dichloro-4,5,6,7-tetrahydrobenzothiophen-5-amine (219)

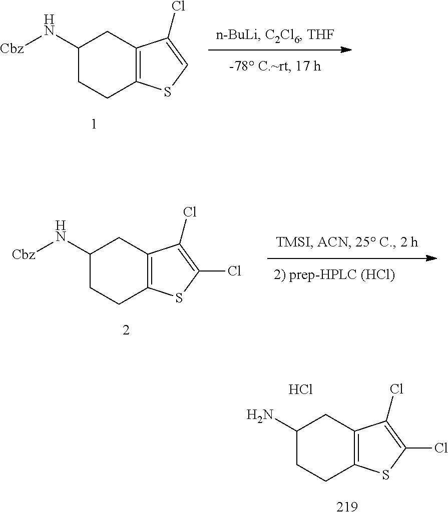 [SMILES: O=C(NC1CCc2scc(Cl)c2C1)OCc1ccccc1 ; O=C(NC1CCc2sc(Cl)c(Cl)c2C1)OCc1ccccc1 ; NC1CCc2sc(Cl)c(Cl)c2C1 ; Cl]

### Procedure for preparation of benzyl N-(2,3-dichloro-4,5,6,7-tetrahydrobenzothiophen-5-yl)carbamate (2

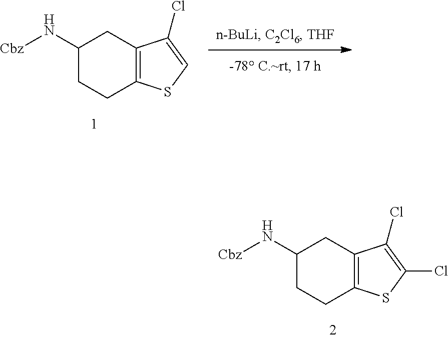 [SMILES: O=C(NC1CCc2scc(Cl)c2C1)OCc1ccccc1 ; O=C(NC1CCc2sc(Cl)c(Cl)c2C1)OCc1ccccc1]

To a solution of benzyl N-(3-chloro-4,5,6,7-tetrahydrobenzothiophen-5-yl)carbamate (1) (300 mg, 932.19 μmol, 1 eq) in THF (4 mL) was added n-BuLi (2.5 M, 932.19 μL, 2.5 eq) drop-wise at −78° C. over 0.5 hr. After addition, the mixture was stirred at this temperature for 0.5 hr, and then 1,1,1,2,2,2-hexachloroethane (264.82 mg, 1.12 mmol, 126.65 μL, 1.2 eq) in THF (1 mL) was added dropwise. The resulting mixture was stirred at −78° C. for 16 hr and then poured into NH₄Cl saturated aq.solution (20 mL). The resulting mixture was extracted with EtOAc (20 mL*2). The combined organic phases were washed with brine (10 mL), dried over anhydrous sodium sulfate, filtered and concentrated under vacuum. The residue was purified by flash silica gel chromatography (ISCO®; 4 g SepaFlash® Silica Flash Column, eluent 0~20% Ethyl acetate/Petroleum ether gradient @20 mL/min) to afford benzyl N-(2,3-dichloro-4,5,6,7-tetrahydrobenzothiophen-5-yl) carbamate (2) (210 mg, 589.45 μmol, 63.23% yield) as yellow oil. ¹H NMR (400 MHz, CHLOROFORM-d) δ=7.41-7.29 (m, 5H), 5.12 (s, 2H), 4.90-4.75 (m, 1H), 4.13-4.04 (m, 1H), 2.91 (br dd, J=4.8, 16.3 Hz, 1H), 2.84-2.70 (m, 2H), 2.38 (br dd, J=7.0, 16.5 Hz, 1H), 2.15-2.06 (m, 1H), 1.95-1.80 (m, 1H).

### Procedure for preparation of 2,3-dichloro-4,5,6,7-tetrahydrobenzothiophen-5-amine hydrochloride (219)

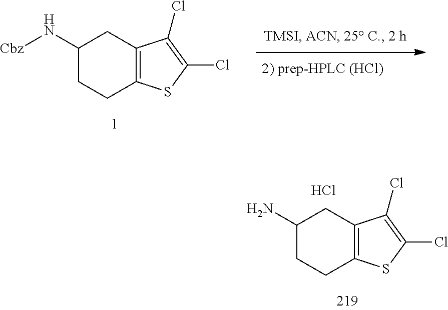 [SMILES: O=C(NC1CCc2sc(Cl)c(Cl)c2C1)OCc1ccccc1 ; NC1CCc2sc(Cl)c(Cl)c2C1 ; Cl]

To a solution of benzyl N-(2,3-dichloro-4,5,6,7-tetrahydrobenzothiophen-5-yl) carbamate (1) (80 mg, 224.55 μmol, 1 eq) in ACN (1 mL) was added TMSI (179.72 mg, 898.20 μmol, 122.26 μL, 4 eq). The mixture was stirred at 25° C. for 2 hr and then concentrated. The residue was purified by prep-HPLC (column: Phenomenex luna C18 150*25 mm*10 μm; mobile phase: [water(HCl)-ACN]; gradient:5%-35% B over 10 min) to afford 2,3-dichloro-4,5,6,7-tetrahydrobenzothiophen-5-amine (219) (22.34 mg, 84.66 μmol, 37.70% yield) as a white solid.LC-MS: m/z [M+H]⁺=222.0. ¹H NMR (400 MHz, METHANOL-d4) δ=3.63 (dddd, J=2.8, 5.7, 8.4, 11.1 Hz, 1H), 3.01 (dd, J=5.1, 15.9 Hz, 1H), 2.94-2.82 (m, 2H), 2.53 (br dd, J=9.0, 16.1 Hz, 1H), 2.26 (td, J=4.1, 8.5 Hz, 1H), 2.02-1.88 (m, 1H)

### Synthetic scheme for preparation of 2,3-dichloro-N-methyl-4,5,6,7-tetrahydrobenzothiophen-5-amine hydrochloride (220)

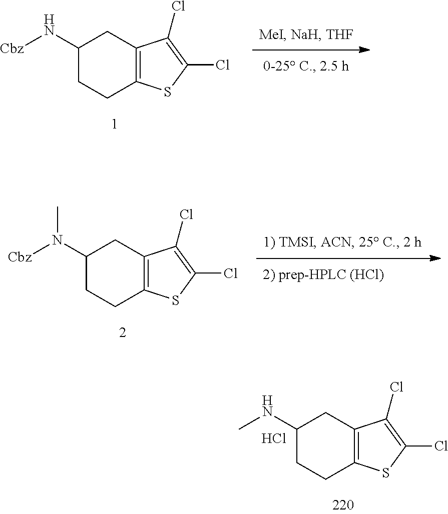 [SMILES: O=C(NC1CCc2sc(Cl)c(Cl)c2C1)OCc1ccccc1 ; CN(C(=O)OCc1ccccc1)C1CCc2sc(Cl)c(Cl)c2C1 ; CNC1CCc2sc(Cl)c(Cl)c2C1 ; Cl]

### Procedure for preparation of benzyl N-(2,3-dichloro-4,5,6,7-tetrahydrobenzothiophen-5-yl)-N-methyl-carbamate (2)

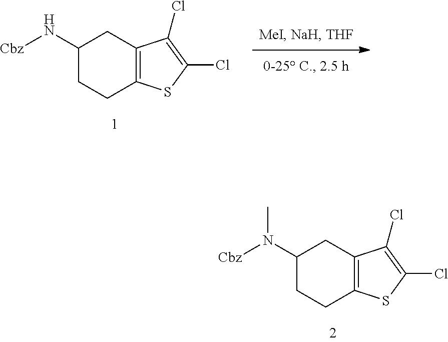 [SMILES: O=C(NC1CCc2sc(Cl)c(Cl)c2C1)OCc1ccccc1 ; CN(C(=O)OCc1ccccc1)C1CCc2sc(Cl)c(Cl)c2C1]

To a solution of benzyl N-(2,3-dichloro-4,5,6,7-tetrahydrobenzothiophen-5-yl)carbamate (1) (120 mg, 336.83 μmol, 1 eq) in DMF (1 mL) was added NaH (20.21 mg, 505.24 μmol, 60% suspension in mineral oil, 1.5 eq) at 0° C. over 15 min. After addition, the mixture was stirred at this temperature for 15 min, and then CH₃I (71.71 mg, 505.24 μmol, 31.45 μL, 1.5 eq) was added dropwise at 0° C. The resulting mixture was warmed up to 25° C. and stirred for 2 hr. The reaction mixture was then poured into NH₄Cl aq. saturated solution (10 mL) and extracted with EtOAc (20 mL*2). The combined organic phases were washed with brine (10 mL), dried over anhydrous sodium sulfate, filtered and concentrated under vacuum. The residue was purified by prep-TLC (SiO₂, Petroleum ether:Ethyl acetate=3:1) to afford benzyl N-(2,3-dichloro-4,5,6,7-tetrahydrobenzothiophen-5-yl)-N-methyl-carbamate (2) (80 mg, 216.05 μmol, 64.14% yield) as yellow oil. ¹H NMR (400 MHz, CHLOROFORM-d) δ=7.44-7.29 (m, 5H), 5.17 (s, 2H), 4.57-4.27 (m, 1H), 2.89 (s, 3H), 2.83 (br s, 2H), 2.78-2.64 (m, 1H), 2.59-2.47 (m, 1H), 2.03-1.89 (m, 2H)

### Procedure for preparation of 2,3-dichloro-N-methyl-4,5,6,7-tetrahydrobenzothiophen-5-amine hydrochloride (220)

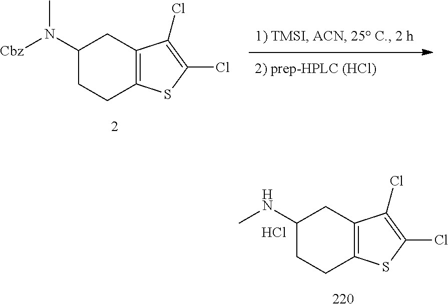 [SMILES: CN(C(=O)OCc1ccccc1)C1CCc2sc(Cl)c(Cl)c2C1 ; CNC1CCc2sc(Cl)c(Cl)c2C1 ; Cl]

To a solution of benzyl N-(2,3-dichloro-4,5,6,7-tetrahydrobenzothiophen-5-yl)-N-methyl-carbamate (2) (80 mg, 216.05 μmol, 1 eq) in ACN (1 mL) was added TMSI (172.92 mg, 864.18 μmol, 117.63 μL, 4 eq). The mixture was stirred at 25° C. for 2 hr and then concentrated. The residue was purified by prep-HPLC (column: Phenomenex luna C18 150*25 mm*10 μm; mobile phase: [water(HCl)-ACN]; gradient:5%-35% B over 10 min) to afford 2,3-dichloro-N-methyl-4,5,6,7-tetrahydrobenzothiophen-5-amine hydrochloride (220) (39.07 mg, 140.45 μmol, 65.01% yield) as a yellow solid. ¹H NMR (400 MHz, METHANOL-d4) δ=3.62-3.51 (m, 1H), 3.08 (dd, J=5.3, 15.8 Hz, 1H), 2.96-2.86 (m, 2H), 2.81 (s, 3H), 2.62-2.52 (m, 1H), 2.41-2.30 (m, 1H), 2.02-1.89 (m, 1H). LC-MS: m/z [M+H]⁺=236.0.

### Synthetic scheme for preparation of 3-(trifluoromethyl)-6,7-dihydro-5H-thieno[3,2-b]pyran-6-amine hydrochloride (221)

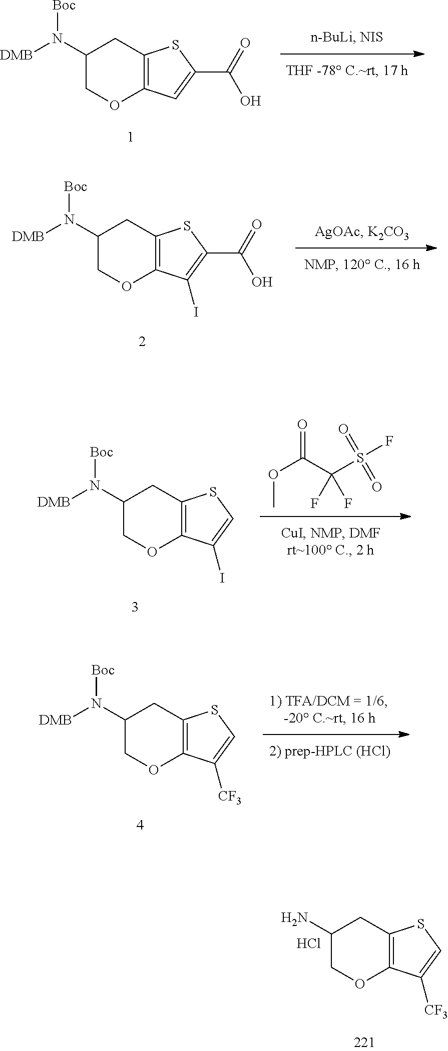

### Procedure for preparation of 6-[tert-butoxycarbonyl-[(2,4-dimethoxyphenyl)methyl]amino]-3-iodo-6,7-dihydro-5H-thieno[3,2-b]pyran-2-carboxylic acid (2)

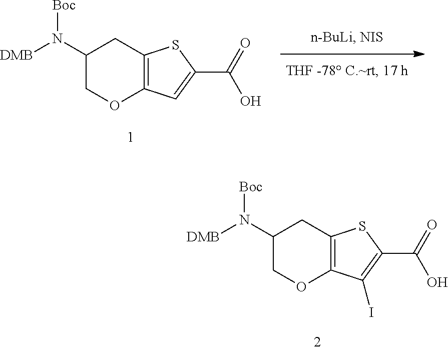

To a solution of 6-[tert-butoxycarbonyl-[(2,4-dimethoxyphenyl)methyl]amino]-6,7-dihydro-5H-thieno[3,2-b]pyran-2-carboxylic acid (1) (400 mg, 889.85 μmol, 1 eq) in THF (5 mL) was added dropwise n-BuLi (2.5 M, 854.25 μL, 2.4 eq) at −78° C. over 0.5 hr. After addition, the mixture was stirred at this temperature for 0.5 hr, and then NIS (300.30 mg, 1.33 mmol, 1.5 eq) in THF (2 mL) was added dropwise at −78° C. The resulting mixture was stirred at −78° C. for 16 hr. The reaction mixture was poured into NH₄Cl saturated aq. solution (20 mL) and extracted with EtOAc (20 mL*2). The combined organic phases were washed with brine (10 mL), dried over anhydrous sodium sulfate, filtered and concentrated under vacuum to give a residue. The crude product 6-[tert-butoxycarbonyl-[(2,4-dimethoxyphenyl)methyl]amino]-3-iodo-6,7-dihydro-5H-thieno[3,2-b]pyran-2-carboxylic acid (2) (500 mg, crude) was used into the next step without further purification.

### Procedure for preparation of tert-butyl N-[(2,4-dimethoxyphenyl)methyl]-N-(3-iodo-6,7-dihydro-5H-thieno[3,2-b]pyran-6-yl)carbamate (3)

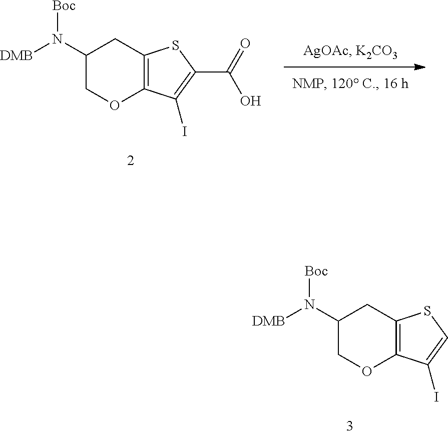

To a solution of 6-[tert-butoxycarbonyl-[(2,4-dimethoxyphenyl)methyl]amino]-3-iodo-6,7-dihydro-5H-thieno[3,2-b]pyran-2-carboxylic acid (2) (500 mg, 868.94 μmol, 1 eq) in NMP (5 mL) was added AgOAc (43.51 mg, 260.68 μmol, 0.3 eq) and K₂CO₃ (360.28 mg, 2.61 mmol, 3 eq). The mixture was stirred at 120° C. for 16 hr and then poured into H₂O (50 mL). Followed extraction with EtOAc (20 mL*2). The combined organic phases were washed with brine (10 mL), dried over anhydrous sodium sulfate, filtered and concentrated under vacuum. The residue was purified by flash silica gel chromatography (ISCO®; 12 g SepaFlash® Silica Flash Column, eluent 0~100% Ethyl acetate/Petroleum ether gradient @40 mL/min) to afford tert-butyl N-[(2,4-dimethoxyphenyl)methyl]-N-(3-iodo-6,7-dihydro-5H-thieno[3,2-b]pyran-6-yl)carbamate (3) (77 mg, 144.90 μmol, 16.68% yield) as yellow oil. ¹H NMR (400 MHz, CHLOROFORM-d) 7.15-7.05 (m, 2H), 6.49-6.41 (m, 2H), 4.52-3.99 (m, 5H), 3.80 (d, J=12.9 Hz, 6H), 3.22-3.05 (m, 1H), 2.83-2.66 (m, 1H), 1.55-1.47 (m, 9H)

### Procedure for preparation of tert-butyl N-[(2,4-dimethoxyphenyl)methyl]-N-[3-(trifluoromethyl)-6,7-dihydro-5H-thieno[3,2-b]pyran-6-yl]carbamate (4)

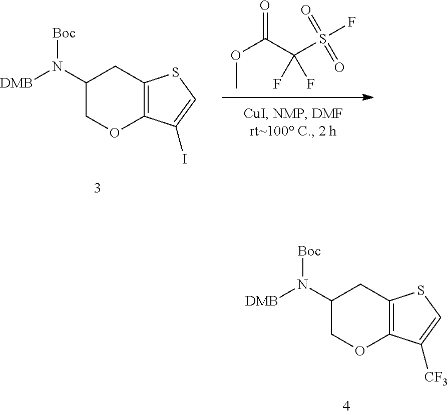

To a solution of tert-butyl N-[(2,4-dimethoxyphenyl)methyl]-N-(3-iodo-6,7-dihydro-5H-thieno[3,2-b]pyran-6-yl)carbamate (3) (77 mg, 144.90 μmol, 1 eq) in DMF (2 mL) was added CuI (30.36 mg, 159.39 μmol, 1.1 eq) and NMP (71.82 mg, 724.50 μmol, 70.41 μL, 5 eq) at 25° C. The mixture was stirred for 15 min, and then methyl 2,2-difluoro-2-fluorosulfonyl-acetate (139.19 mg, 724.50 μmol, 92.18 μL, 5 eq) was added dropwise at 25° C. The resulting mixture was then stirred at 100° C. for 2 hr then cooled, filtered and concentrated. The residue was purified by prep-HPLC (column: C18 150×30 mm; mobile phase: [water(FA)-ACN]; gradient:70%-100% B over 7 min) to afford tert-butyl N-[(2,4-dimethoxyphenyl)methyl]-N-[3-(trifluoromethyl)-6,7-dihydro-5H-thieno[3,2-b]pyran-6-yl]carbamate (4) (30 mg, 63.36 μmol, 43.73% yield) as a yellow solid. ¹H NMR (400 MHz, CHLOROFORM-d) δ=7.37 (s, 1H), 7.17-7.08 (m, 1H), 6.50-6.42 (m, 2H), 4.52-4.07 (m, 5H), 3.80 (d, J=12.9 Hz, 6H), 3.21-3.04 (m, 1H), 2.80-2.68 (m, 1H), 1.48 (br s, 9H)

### Procedure for preparation of 3-(trifluoromethyl)-6,7-dihydro-5H-thieno[3,2-b]pyran-6-amine hydrochloride (221)

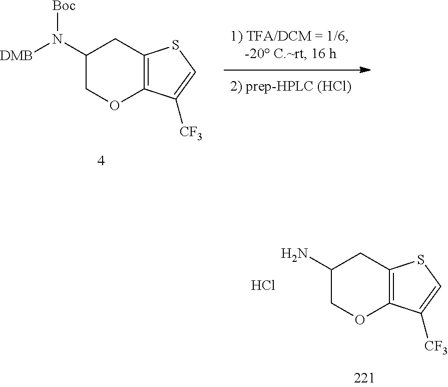

To a solution of tert-butyl N-[(2,4-dimethoxyphenyl)methyl]-N-[3-(trifluoromethyl)-6,7-dihydro-5H-thieno[3,2-b]pyran-6-yl]carbamate (4) (30 mg, 63.36 μmol, 1 eq) in DCM (0.6 mL) was added TFA (153.50 mg, 1.35 mmol, 100.00 μL, 21.25 eq) at −20° C. The mixture was stirred at −20-25° C. for 16 hr and then concentrated. The residue was purified by prep-HPLC (column: Phenomenex luna C18 150*25 mm*10 μm; mobile phase: [water(HCl)-ACN]; gradient:1%-25% B over 10 min) to afford 3-(trifluoromethyl)-6,7-dihydro-5H-thieno[3,2-b]pyran-6-amine hydrochloride (221) (14.27 mg, 53.30 μmol, 84.13% yield) as an off-white solid. ¹H NMR (400 MHz, METHANOL-d4) δ=7.81 (s, 1H), 4.46-4.38 (m, 1H), 4.32-4.25 (m, 1H), 3.98 (br d, J=1.4 Hz, 1H), 3.39-3.32 (m, 1H), 2.94 (br d, J=17.4 Hz, 1H). LC-MS: m/z [M+H]⁺=224.0.

### Synthetic scheme for preparation of 3-chloro-N-methyl-6,7-dihydro-5H-thieno[3,2-b]pyran-6-amine hydrochloride (222)

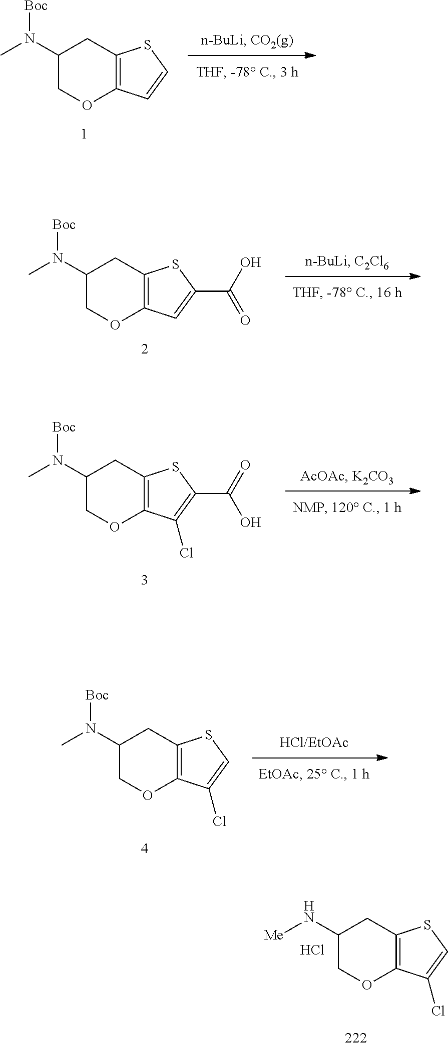 [SMILES: CN(C(=O)OC(C)(C)C)C1COc2ccsc2C1 ; CN(C(=O)OC(C)(C)C)C1COc2cc(C(=O)O)sc2C1 ; CN(C(=O)OC(C)(C)C)C1COc2c(sc(C(=O)O)c2Cl)C1 ; CN(C(=O)OC(C)(C)C)C1COc2c(Cl)csc2C1 ; CNC1COc2c(Cl)csc2C1 ; Cl]

### Procedure for preparation of 6-[tert-butoxycarbonyl(methyl)amino]-6,7-dihydro-5H-thieno[3,2-b]pyran-2-carboxylic acid (2)

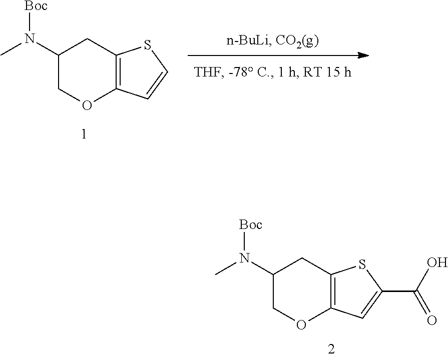 [SMILES: CN(C(=O)OC(C)(C)C)C1COc2ccsc2C1 ; CN(C(=O)OC(C)(C)C)C1COc2cc(C(=O)O)sc2C1]

To the solution of tert-butyl N-(6,7-dihydro-5H-thieno[3,2-b]pyran-6-yl)-N-methyl-carbamate (1) (2 g, 7.43 mmol, 1 eq) in THF (20 mL) was added n-BuLi (2.5 M, 7.13 mL, 2.4 eq) at −78° C. After stirring for 1 h, CO₂ (2 L) was bubbled at −78° C. and the resulting mixture was stirred for 15 h at 20° C. The reaction mixture was quenched by addition sat. aq. NH₄Cl (10 mL) and extracted with DCM 20 (10 mL*2). The combined organic layers were washed with brine 10 mL, dried over Na₂SO₄, filtered and concentrated under reduced pressure. The residue was purified by flash silica gel chromatography (ISCO®; 40 g SepaFlash® Silica Flash Column, eluent 0~60% Ethyl acetate/Petroleum ether gradient @60 mL/min). 6-[Tert-butoxycarbonyl(methyl)amino]-6,7-dihydro-5H-thieno[3,2-b]pyran-2-carboxylic acid (2) (860 mg, 2.74 mmol, 36.96% yield) was obtained as a yellow solid. LCMS: m/z [M+H-tBu]⁺=258.

### Procedure for preparation of 6-[tert-butoxycarbonyl(methyl)amino]-3-chloro-6,7-dihydro-5H-thieno[3,2-b]pyran-2-carboxylic acid (3)

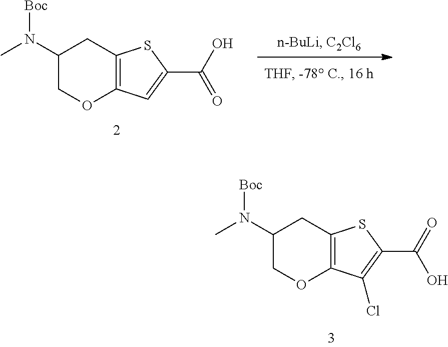 [SMILES: CN(C(=O)OC(C)(C)C)C1COc2cc(C(=O)O)sc2C1 ; CN(C(=O)OC(C)(C)C)C1COc2c(sc(C(=O)O)c2Cl)C1]

To a solution of 6-[tert-butoxycarbonyl(methyl)amino]-6,7-dihydro-5H-thieno[3,2-b]pyran-2-carboxylic acid (2) (500 mg, 1.60 mmol, 1 eq) in THF (5 mL) was added dropwise n-BuLi (2.5 M, 1.53 mL, 2.4 eq) at −78° C. over 0.5 hr. After addition, the mixture was stirred at this temperature for 0.5 hr, and then 1,1,1,2,2,2-hexachloroethane (566.60 mg, 2.39 mmol, 270.97 μL, 1.5 eq) in THF (1 mL) was added dropwise at −78° C. The resulting mixture was stirred allowed to gradually warm up to 25° C., stirred for 16 hr and then poured into aq. NH₄Cl saturated solution (20 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 40 g SepaFlash® Silica Flash Column, eluent 0~100% Ethyl acetate/Petroleum ether gradient @100 mL/min). 6-[Tert-butoxycarbonyl(methyl)amino]-3-chloro-6,7-dihydro-5H-thieno[3,2-b]pyran-2-carboxylic acid (3) (60 mg, 115.00 μmol, 7.21% yield) was obtained as yellow oil. LCMS: m/z [M+H-tBu]⁺=292.1.

### Procedure for preparation of tert-butyl N-(3-chloro-6,7-dihydro-5H-thieno[3,2-b]pyran-6-yl)-N-methyl-carbamate (4)

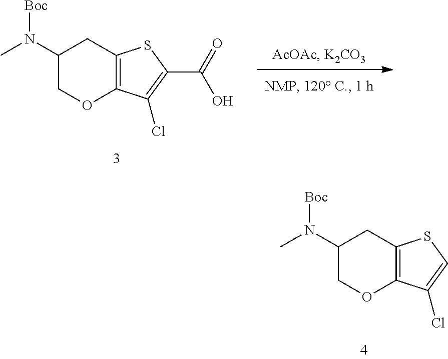 [SMILES: CN(C(=O)OC(C)(C)C)C1COc2c(sc(C(=O)O)c2Cl)C1 ; CN(C(=O)OC(C)(C)C)C1COc2c(Cl)csc2C1]

To a solution of 6-[tert-butoxycarbonyl(methyl)amino]-3-chloro-6,7-dihydro-5H-thieno[3,2-b]pyran-2-carboxylic acid (3) (60 mg, 172.51 μmol, 1 eq) in NMP (2 mL) was added AgOAc (14.40 mg, 86.25 μmol, 0.5 eq) and K₂CO₃ (71.52 mg, 517.52 μmol, 3 eq). The mixture was stirred at 120° C. for 1 hr then cooled and filtered. Then filtrate was diluted with H₂O (10 ml) and extracted with EtOAc 20 mL (10 mL*2). The combined organic layers were washed with aq. sat. NaCl (10 mL*3), dried over Na₂SO₄, filtered and concentrated under reduced pressure. The residue was purified by prep-TLC (SiO₂, Petroleum ether/Ethyl acetate=5/1). Compound tert-butyl N-(3-chloro-6,7-dihydro-5H-thieno[3,2-b]pyran-6-yl)-N-methyl-carbamate (4) (20 mg, 65.83 μmol, 38.16% yield) was obtained as an oil. LCMS: m/z [M+H-tBu]⁺=247.9.

### Procedure for preparation of 3-chloro-N-methyl-6,7-dihydro-5H-thieno[3,2-b]pyran-6-amine hydrochloride (222)

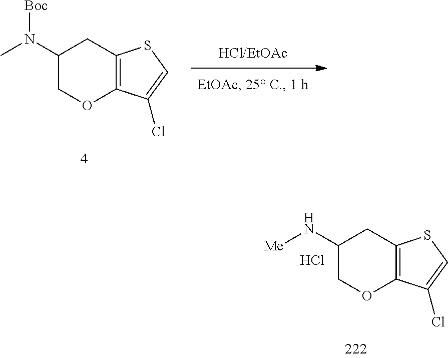 [SMILES: CN(C(=O)OC(C)(C)C)C1COc2c(Cl)csc2C1 ; CNC1COc2c(Cl)csc2C1 ; Cl]

To a solution of tert-butyl N-(3-chloro-6,7-dihydro-5H-thieno[3,2-b]pyran-6-yl)-N-methyl-carbamate (4) (20 mg, 65.83 μmol, 1 eq) in EtOAc (1 mL) was added HCl/EtOAc (4 M, 2 mL, 121.52 eq). The mixture was stirred at 25° C. for 1 hr and then concentrated to dryness. The residue was triturated with EtOAc at 25° C. for 10 min and filtered. The filter cake was collected and dried to afford the product. Compound 3-chloro-N-methyl-6,7-dihydro-5H-thieno[3,2-b]pyran-6-amine hydrochloride (222) (10.30 mg, 42.49 μmol, 64.54% yield) was obtained as an orange solid. ¹H NMR (400 MHz, METHANOL-d₄) δ=7.23 (s, 1H), 4.60 (td, J=2.7, 12.1 Hz, 1H), 4.27 (d, J=13.1 Hz, 1H), 3.86 (br d, J=1.9 Hz, 1H), 3.36 (br d, J=5.8 Hz, 1H), 3.03 (br d, J=17.8 Hz, 1H), 2.82 (s, 3H) LCMS: m/z [M+H]⁺=204.

### Synthetic scheme for preparation of N, 2-dimethyl-4,5,6,7-tetrahydrobenzothiophen-5-amine hydrochloride enantiomer 1 (223) and enantiomer 2 (224)

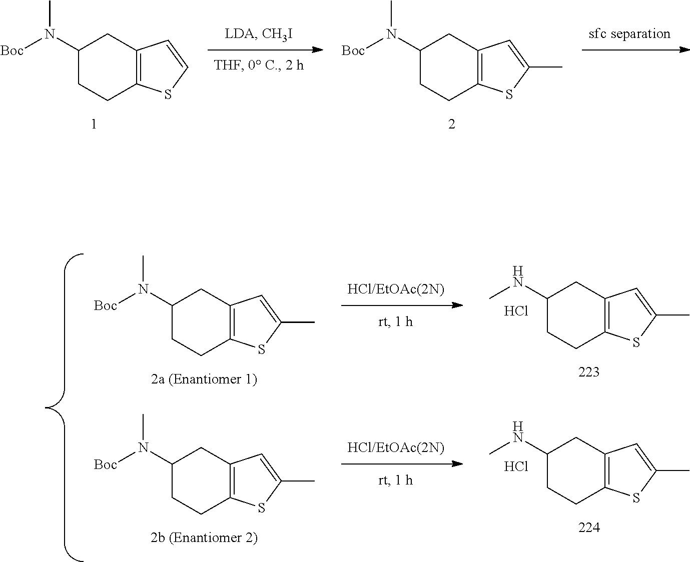

### Procedure for preparation of tert-butyl N-methyl-N-(2-methyl-4,5,6,7-tetrahydrobenzothiophen-5-yl)carbamate (2)

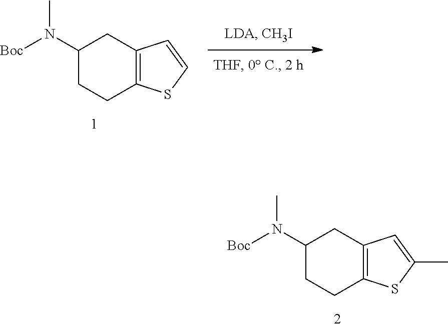 [SMILES: CN(C(=O)OC(C)(C)C)C1CCc2sccc2C1 ; Cc1cc2c(s1)CCC(N(C)C(=O)OC(C)(C)C)C2]

To a solution of tert-butyl N-methyl-N-(4,5,6,7-tetrahydrobenzothiophen-5-yl)carbamate (1) (350 mg, 1.31 mmol, 1 eq) in THF (10 mL) was added dropwise LDA (2 M, 785.38 μL, 1.2 eq) at −78° C. After addition, the mixture was warmed up to 0° C. and stirred for 30 min. Then CH₃I (241.53 mg, 1.70 mmol, 105.93 μL, 1.3 eq) was added dropwise at 0° C. The resulting mixture was stirred at 0° C. for 1.5 hr. The reaction mixture was diluted with aq. sat. NH₄Cl (10 mL) and extracted with EtOAc (30 mL*2). The combined organic layers were washed with aq. sat. NaCl (10 mL), dried over anhydrous Na₂SO₄, filtered and concentrated under reduced pressure to give a residue. The residue was purified by prep-HPLC (column: Phenomenex luna C18 150*40 mm*15 μm; mobile phase: [water(FA)-ACN]; gradient:58%-88% B over 15 min). Compound tert-butyl N-methyl-N-(2-methyl-4,5,6,7-tetrahydrobenzothiophen-5-yl)carbamate (2) (150 mg, 533.02 μmol, 40.72% yield) was obtained as a yellow solid. ¹H NMR (400 MHz, CHLOROFORM-d) δ=6.39 (s, 1H), 4.49-4.19 (m, 1H), 2.88-2.77 (m, 5H), 2.71-2.57 (m, 2H), 2.41 (s, 3H), 1.97-1.86 (m, 2H), 1.48 (s, 9H). LC-MS: m/z [M+H-tBu]⁺=226.1.

### Procedure for preparation of tert-butyl N-methyl-N-(2-methyl-4,5,6,7-tetrahydrobenzothiophen-5-yl)carbamate enantiomer 1 (2a) and enantiomer 2 (2b)

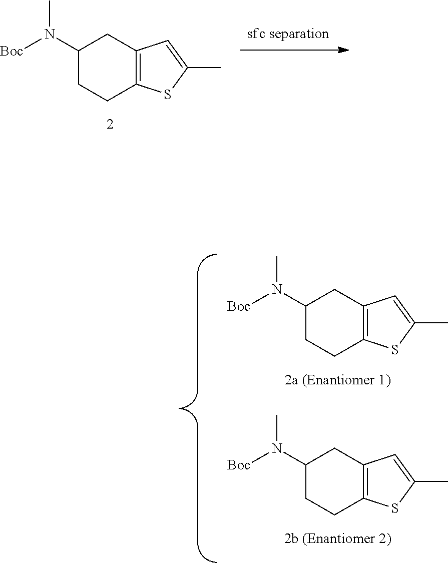 [SMILES: Cc1cc2c(s1)CCC(N(C)C(=O)OC(C)(C)C)C2 ; Cc1cc2c(s1)CCC(N(C)C(=O)OC(C)(C)C)C2 ; Cc1cc2c(s1)CCC(N(C)C(=O)OC(C)(C)C)C2]

Racemic tert-butyl N-methyl-N-(2-methyl-4,5,6,7-tetrahydrobenzothiophen-5-yl)carbamate (2) was separated to enantiomers by SFC (column: DAICEL CHIRALPAK AD (250 mm*50 mm, 10 μm); mobile phase: [CO₂-MeOH (0.1% NH₃H₂O)]; B %:10%, isocratic elution mode). Compound tert-butyl N-methyl-N-(2-methyl-4,5,6,7-tetrahydrobenzothiophen-5-yl)carbamate enantiomer 1 (2a) (65 mg, 230.98 μmol, 43.33% yield) was obtained as a yellow oil. Compound tert-butyl N-methyl-N-(2-methyl-4,5,6,7-tetrahydrobenzothiophen-5-yl)carbamate enantiomer (2b) (80 mg, 284.28 μmol, 53.33% yield) was obtained as a yellow oil.

### Procedure for preparation of N, 2-dimethyl-4,5,6,7-tetrahydrobenzothiophen-5-amine hydrochloride enantiomer 1 (223)

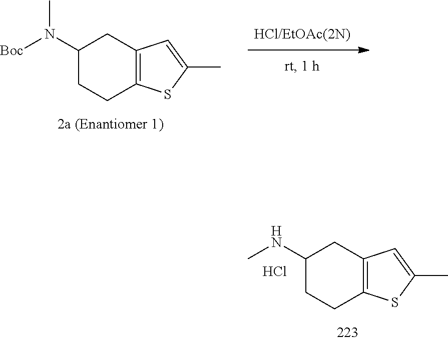

To a solution of tert-butyl N-methyl-N-(2-methyl-4,5,6,7-tetrahydrobenzothiophen-5-yl)carbamate enantiomer 1 (2a) (65 mg, 230.98 μmol, 1 eq) in EtOAc (2 mL) was added HCl/EtOAc (4 M, 2 mL, 28.14 eq). The mixture was stirred at 25° C. for 1 hr and filtered. The solid obtained was washed with EtOAc (10 mL) dried and collected. Compound N, 2-dimethyl-4,5,6,7-tetrahydrobenzothiophen-5-amine hydrochloride enantiomer 1 (223) (46.54 mg, 208.57 μmol, 90.30% yield) was obtained as a white solid. ¹H NMR (400 MHz, METHANOL-d4) δ=6.46 (s, 1H), 3.55-3.41 (m, 1H), 3.07 (br dd, J=5.0, 15.6 Hz, 1H), 2.95-2.85 (m, 2H), 2.78 (s, 3H), 2.69-2.57 (m, 1H), 2.38 (s, 3H), 2.34-2.25 (m, 1H), 2.01-1.86 (m, 1H). LC-MS: m/z [M+H]⁺=182.2

### Procedure for preparation of N, 2-dimethyl-4,5,6,7-tetrahydrobenzothiophen-5-amine hydrochloride enantiomer 2 (224)

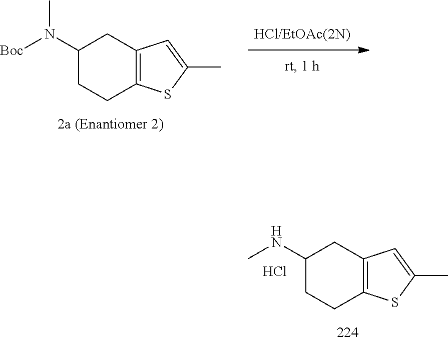

To a solution of tert-butyl N-methyl-N-(2-methyl-4,5,6,7-tetrahydrobenzothiophen-5-yl)carbamate enantiomer 2 (2b) (80 mg, 284.28 μmol, 1 eq) in EtOAc (2 mL) was added HCl/EtOAc (4 M, 2.00 mL, 28.14 eq). The mixture was stirred at 25° C. for 1 hr and then filtered. The filter cake was washed with EtOAc (10 mL) dried and collected. Compound N, 2-dimethyl-4,5,6,7-tetrahydrobenzothiophen-5-amine hydrochloride enantiomer 2 (224) (58.06 mg, 258.31 μmol, 90.86% yield) was obtained as a white solid. ¹H NMR (400 MHz, METHANOL-d4) δ=6.46 (s, 1H), 3.52-3.42 (m, 1H), 3.07 (dd, J=4.6, 15.8 Hz, 1H), 2.93-2.83 (m, 2H), 2.77 (s, 3H), 2.62 (br dd, J=9.1, 15.8 Hz, 1H), 2.38 (s, 3H), 2.34-2.25 (m, 1H), 1.93 (dddd, J=6.8, 8.9, 10.6, 12.8 Hz, 1H). LC-MS: m/z [M+H]+=182.2.

### Synthetic scheme for preparation of 3-fluoro-N-methyl-6,7-dihydro-5H-thieno[3,2-b]pyran-6-amine hydrochloride (225)

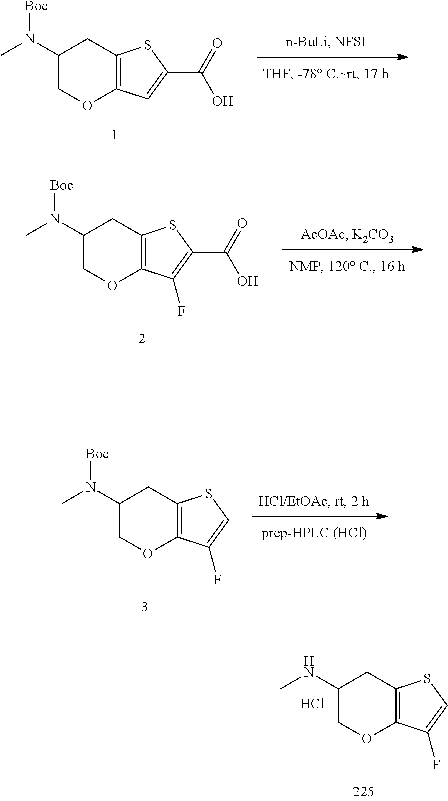 [SMILES: CN(C(=O)OC(C)(C)C)C1COc2cc(C(=O)O)sc2C1 ; CN(C(=O)OC(C)(C)C)C1COc2c(sc(C(=O)O)c2F)C1 ; CN(C(=O)OC(C)(C)C)C1COc2c(F)csc2C1 ; CNC1COc2c(F)csc2C1 ; Cl]

### Procedure for preparation of 6-[tert-butoxycarbonyl(methyl)amino]-3-fluoro-6,7-dihydro-5H-thieno[3,2-b]pyran-2-carboxylic acid (1)

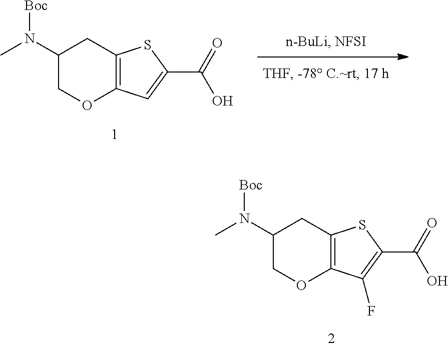 [SMILES: CN(C(=O)OC(C)(C)C)C1COc2cc(C(=O)O)sc2C1 ; CN(C(=O)OC(C)(C)C)C1COc2c(sc(C(=O)O)c2F)C1]

To a solution of 6-[tert-butoxycarbonyl(methyl)amino]-6,7-dihydro-5H-thieno[3,2-b]pyran-2-carboxylic acid (1) (400 mg, 1.28 mmol, 1 eq) in THF (5 mL) was added dropwise n-BuLi (2.5 M, 1.23 mL, 2.4 eq) at −78° C. over 0.5 hr. After addition, the mixture was stirred at this temperature for 0.5 hr and then NFSI (603.77 mg, 1.91 mmol, 1.5 eq) in THF (4 mL) was added dropwise at −78° C. The resulting mixture was gradually warmed up to 25° C. and stirred for 16 hr. The reaction mixture then was poured into aq. NH₄Cl saturated solution (20 mL) and extracted with EtOAc (20 mL*2). The combined organic phase was washed with brine (10 mL), dried over anhydrous sodium sulfate, filtered and concentrated under vacuum to give 6-[tert-butoxycarbonyl(methyl)amino]-3-fluoro-6,7-dihydro-5H-thieno[3,2-b]pyran-2-carboxylic acid (2) as a mixture with the starting material (700 mg, crude) as a brown solid.

### Procedure for preparation of tert-butyl N-(3-fluoro-6,7-dihydro-5H-thieno[3,2-b]pyran-6-yl)-N-methyl-carbamate (10)

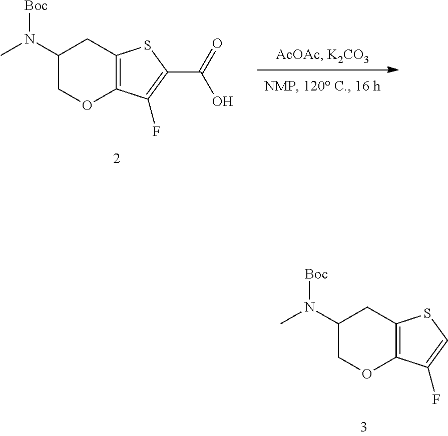 [SMILES: CN(C(=O)OC(C)(C)C)C1COc2c(sc(C(=O)O)c2F)C1 ; CN(C(=O)OC(C)(C)C)C1COc2c(F)csc2C1]

To a solution of crude 6-[tert-butoxycarbonyl(methyl)amino]-3-fluoro-6,7-dihydro-5H-thieno[3,2-b]pyran-2-carboxylic acid (2) (700 mg, 2.11 mmol, 1 eq) in NMP (8 mL) was added AgOAc (70.52 mg, 422.50 μmol, 0.2 eq) and K₂CO₃ (875.88 mg, 6.34 mmol, 3 eq). The mixture was stirred at 120° C. for 16 hr. The reaction mixture was then cooled and poured into H₂O (30 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~100% Ethyl acetate/Petroleum ether gradient @20 mL/min) and by prep-HPLC (column: Phenomenex luna C18 150*25 mm*10 μm; mobile phase: [water(FA)-ACN]; gradient:52%-82% B over 9 min) to give tert-butyl N-(3-fluoro-6,7-dihydro-5H-thieno[3,2-b]pyran-6-yl)-N-methyl-carbamate (3) (50 mg, 174.00 μmol, 8.24% yield) as yellow oil. LC-MS: m/z [M+H-tBu]⁺=232.0

### Procedure for preparation of 3-fluoro-N-methyl-6,7-dihydro-5H-thieno[3,2-b]pyran-6-amine hydrochloride (225)

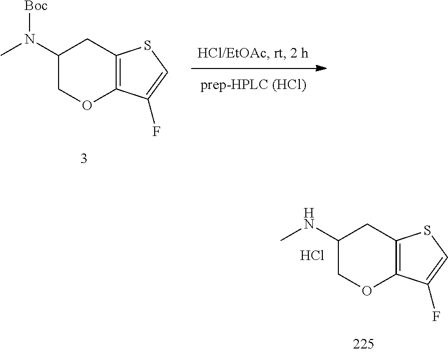 [SMILES: CN(C(=O)OC(C)(C)C)C1COc2c(F)csc2C1 ; CNC1COc2c(F)csc2C1 ; Cl]

To a solution of tert-butyl N-(3-fluoro-6,7-dihydro-5H-thieno[3,2-b]pyran-6-yl)-N-methyl-carbamate (3) (50 mg, 174.00 μmol, 1 eq) in EtOAc (1 mL) was added HCl/EtOAc (4 M, 2.50 mL, 57.47 eq). The mixture was stirred at 25° C. for 2 hr and then was concentrated. The residue was purified by prep-HPLC (column: Phenomenex luna C18 150*25 mm*10 μm; mobile phase: [water(HCl)-ACN]; gradient:1%-15% B over 10 min) to afford 3-fluoro-N-methyl-6,7-dihydro-5H-thieno[3,2-b]pyran-6-amine hydrochloride (225) (10.67 mg, 46.74 μmol, 26.86% yield) as an off-white solid. ¹H NMR (400 MHz, METHANOL-d4) δ=6.83 (s, 1H), 4.58 (br dd, J=1.2, 12.4 Hz, 1H), 4.26 (d, J=12.5 Hz, 1H), 3.86 (br s, 1H), 3.33 (br s, 1H), 3.05-2.93 (m, 1H), 2.82 (s, 3H). LC-MS: m/z [M+H]⁺=188.0

### Synthetic scheme for preparation of 3-fluoro-6,7-dihydro-5H-thieno[3,2-b]pyran-6-amine hydrochloride enantiomer 1 (226) and enantiomer 2 (227)

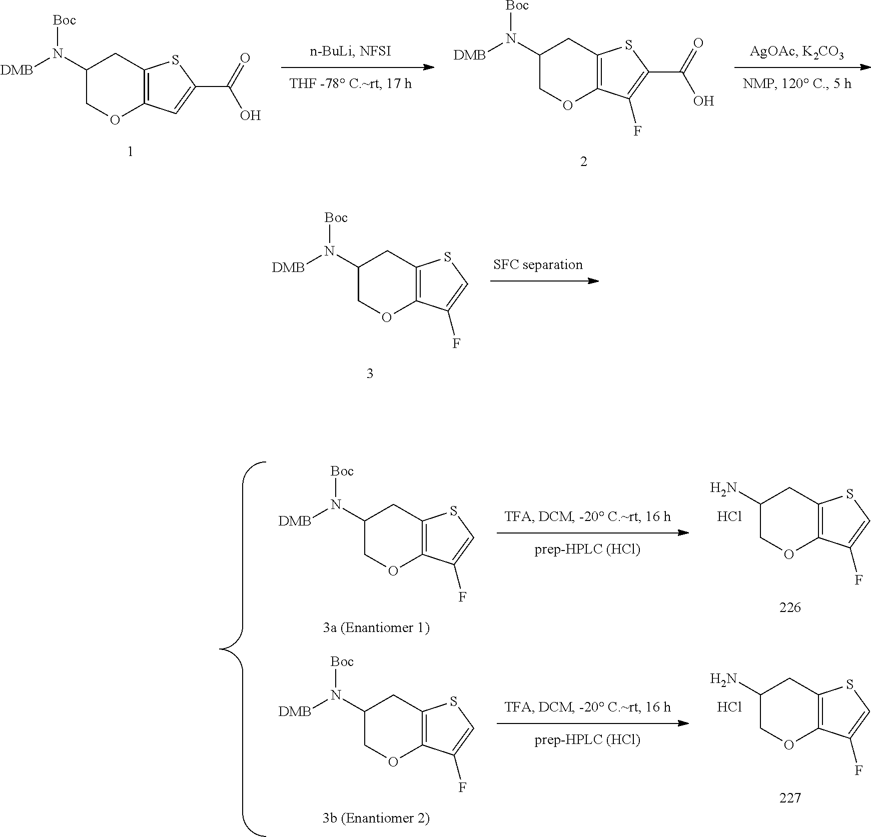

### Procedure for preparation of 6-[tert-butoxycarbonyl-[(2,4-dimethoxyphenyl)methyl]amino]-3-fluoro-6,7-dihydro-5H-thieno[3,2-b]pyran-2-carboxylic acid (2)

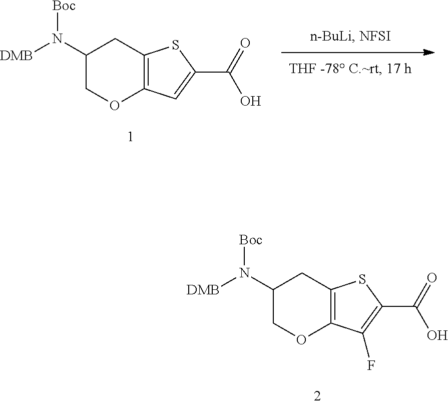

To a solution of 6-[tert-butoxycarbonyl-[(2,4-dimethoxyphenyl)methyl]amino]-6,7-dihydro-5H-thieno[3,2-b]pyran-2-carboxylic acid (1) (400 mg, 889.85 μmol, 1 eq) in THF (5 mL) was added dropwise n-BuLi (2.5 M, 854.25 μL, 2.4 eq) at −78° C. over 0.5 hr. The mixture was stirred at this temperature for 0.5 hr, and then NFSI (420.91 mg, 1.33 mmol, 1.5 eq) in THF (1 mL) was added dropwise at −78° C. The resulting mixture was stirred at −78° C. for 1 h and then at rt for 16 hr. The reaction mixture was then poured into NH₄Cl ag. saturated solution (10 mL) and extracted with EtOAc (20 mL*2). The combined organic phase was washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~20% Ethyl acetate/Petroleum ether gradient @20 mL/min) to afford a mixture of 6-[tert-butoxycarbonyl-[(2,4-dimethoxyphenyl)methyl]amino]-3-fluoro-6,7-dihydro-5H-thieno[3,2-b]pyran-2-carboxylic acid (2) and starting material (1) (360 mg)) as yellow oil.

### Procedure for preparation of tert-butyl N-[(2,4-dimethoxyphenyl)methyl]-N-(3-fluoro-6,7-dihydro-5H-thieno[3,2-b]pyran-6-yl)carbamate (3)

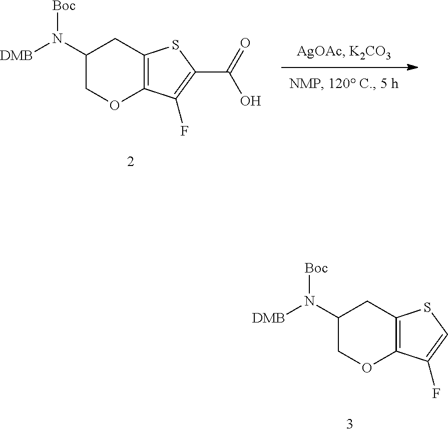

To a solution of the mixture of 6-[tert-butoxycarbonyl-[(2,4-dimethoxyphenyl)methyl]amino]-3-fluoro-6,7-dihydro-5H-thieno[3,2-b]pyran-2-carboxylic acid (2) and 6-[tert-butoxycarbonyl-[(2,4-dimethoxyphenyl)methyl]amino]-6,7-dihydro-5H-thieno[3,2-b]pyran-2-carboxylic acid (1) (300 mg, 641.70 μmol, 1 eq) in NMP (5 mL) was added AgOAc (21.42 mg, 128.34 μmol, 0.2 eq) and K₂CO₃ (266.06 mg, 1.93 mmol, 3 eq). The mixture was stirred at 120° C. for 5 hr then cooled and poured into H₂O (20 mL) and extracted with EtOAc (20 mL*2). The combined organic phases were washed with brine (10 mL), dried over anhydrous sodium sulfate, filtered and concentrated under vacuum. The residue was purified by prep-HPLC (column: C18 150×30 mm; mobile phase: [water-can (50% THF)]; gradient:68%-98% B over 7 min) to afford a mixture of desired product tert-butyl N-[(2,4-dimethoxyphenyl)methyl]-N-(3-fluoro-6,7-dihydro-5H-thieno[3,2-b]pyran-6-yl)carbamate (3) and the decarboxylated product of 6-[tert-butoxycarbonyl-[(2,4-dimethoxyphenyl)methyl]amino]-6,7-dihydro-5H-thieno[3,2-b]pyran-2-carboxylic acid (1) (110 mg) as a yellow solid.

### Procedure for preparation of tert-butyl N-[(2,4-dimethoxyphenyl)methyl]-N-(3-fluoro-6,7-dihydro-5H-thieno[3,2-b]pyran-6-yl)carbamate enantiomer 1 (3a) and enantiomer 2 (3b)

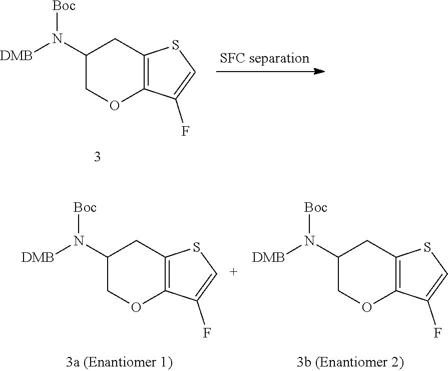

The mixture from the step above was separated by SFC (column: DAICEL CHIRALPAK IG (250 mm*50 mm, 10 μm); mobile phase: [CO₂-i-PrOH (0.1% NH₃H₂O)]; B %:25%, isocratic elution mode) to give desired compound, which was further separated by SFC (column: DAICEL CHIRALPAK IC (250 mm*30 mm, 10 μm); mobile phase: [CO₂-EtOH (0.1% NH₃H₂O)]; B %:27.5%, isocratic elution mode). Compound tert-butyl N-[(2,4-dimethoxyphenyl)methyl]-N-(3-fluoro-6,7-dihydro-5H-thieno[3,2-b]pyran-6-yl)carbamate enantiomer 1 (3a) (13 mg, 30.70 μmol, 11.82% yield) was obtained as yellow oil. Compound tert-butyl N-[(2,4-dimethoxyphenyl)methyl]-N-(3-fluoro-6,7-dihydro-5H-thieno[3,2-b]pyran-6-yl)carbamate enantiomer 2 (3b) (16 mg, 37.78 μmol, 14.55% yield) was obtained as yellow oil. LC-MS: m/z [M+Na]⁺=446.1

### Procedure for preparation of 3-fluoro-6,7-dihydro-5H-thieno[3,2-b]pyran-6-amine hydrochloride enantiomer 1 (226)

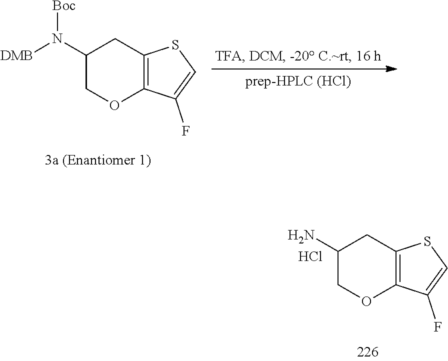

To a solution of tert-butyl N-[(2,4-dimethoxyphenyl)methyl]-N-(3-fluoro-6,7-dihydro-5H-thieno[3,2-b]pyran-6-yl)carbamate enantiomer 1 (3a) (13 mg, 30.70 μmol, 1 eq) in DCM (0.6 mL) was added TFA (199.55 mg, 1.75 mmol, 130.00 μL, 57.01 eq) at −20° C. The mixture was allowed to slowly warm up to 25° C., stirred for 16 hr and then was concentrated. The residue was purified by prep-HPLC (column: Phenomenex luna C18 150*25 mm*10 μm; mobile phase: [water(HCl)-ACN]; gradient:1%-15% B over 10 min) to afford 3-fluoro-6,7-dihydro-5H-thieno[3,2-b]pyran-6-amine hydrochloride enantiomer 1 (226) (5.93 mg, 28.00 μmol, 91.21% yield) as an off-white solid. ¹H NMR (400 MHz, METHANOL-d4) δ=6.82 (s, 1H), 4.41-4.34 (m, 1H), 4.27-4.20 (m, 1H), 3.96 (br d, J=2.6 Hz, 1H), 3.26 (br d, J=5.6 Hz, 1H), 2.84 (br d, J=17.3 Hz, 1H). LC-MS: m/z [M+H]+=174.1

### Procedure for preparation of 3-fluoro-6,7-dihydro-5H-thieno[3,2-b]pyran-6-amine hydrochloride enantiomer 2 (227)

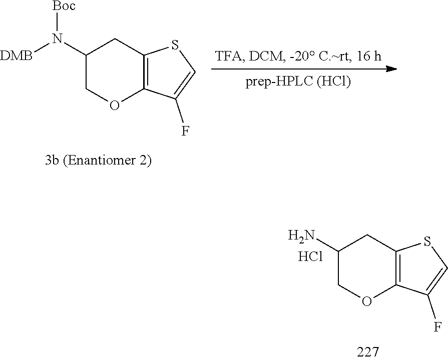

To a solution of tert-butyl N-[(2,4-dimethoxyphenyl)methyl]-N-(3-fluoro-6,7-dihydro-5H-thieno[3,2-b]pyran-6-yl)carbamate enantiomer 2 (3b) (16 mg, 37.78 μmol, 1 eq) in DCM (0.6 mL) was added TFA (153.50 mg, 1.35 mmol, 0.1 mL, 35.63 eq) at−20° C. allowed to slowly warm up to 25° C., stirred for 16 hr and then concentrated The residue was purified by prep-HPLC (column: Phenomenex luna C18 150*25 mm*10 μm; mobile phase: [water(HCl)-ACN]; gradient:1%-15% B over 10 min) to afford 3-fluoro-6,7-dihydro-5H-thieno[3,2-b]pyran-6-amine hydrochloride enantiomer 2 (227) (4.48 mg, 21.15 μmol, 55.99% yield) as an off-white solid. ¹H NMR (400 MHz, METHANOL-d4) δ=6.82 (s, 1H), 4.42-4.35 (m, 1H), 4.24 (dd, J=1.3, 12.1 Hz, 1H), 3.96 (br d, J=2.6 Hz, 1H), 3.26 (d, J=5.6 Hz, 1H), 2.85 (br d, J=17.1 Hz, 1H). LC-MS: m/z [M+H]⁺=174.1

### Synthetic scheme for preparation of 3-methyl-6,7-dihydro-5H-thieno[3,2-b]pyran-6-amine hydrochloride (228)

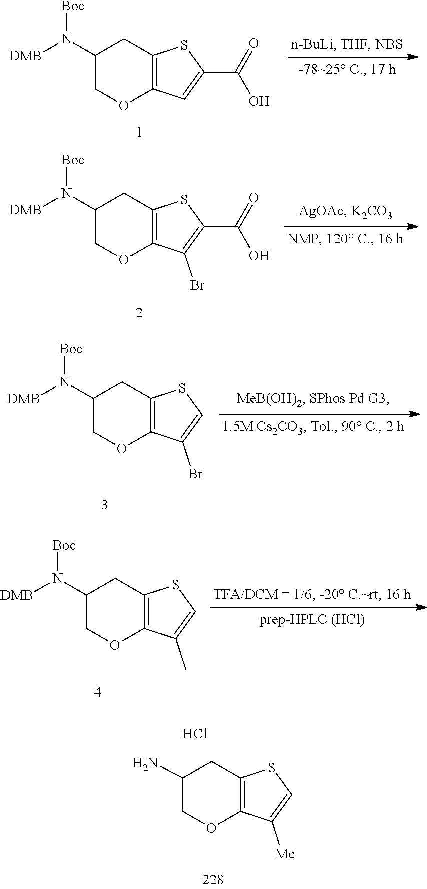

### Procedure for preparation of 3-bromo-6-[tert-butoxycarbonyl-[(2,4-dimethoxyphenyl)methyl]amino]-6,7-dihydro-5H-thieno[3,2-b]pyran-2-carboxylic acid (2)

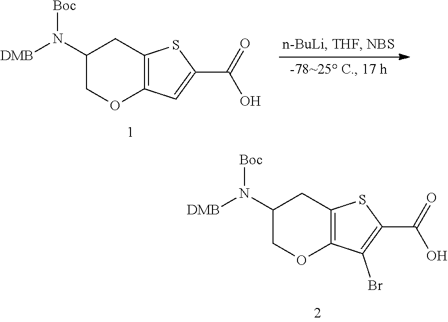

To a solution of 6-[tert-butoxycarbonyl-[(2,4-dimethoxyphenyl)methyl]amino]-6,7-dihydro-5H-thieno[3,2-b]pyran-2-carboxylic acid (1) (1.2 g, 2.67 mmol, 1 eq) in THF (10 mL) was added dropwise n-BuLi (2.5 M, 2.46 mL, 2.3 eq) at −78° C. over 0.5 hr. The mixture was stirred at this temperature for 0.5 hr, and then NBS (617.67 mg, 3.47 mmol, 1.3 eq) in THF (2 mL) was added dropwise at −78° C. The resulting mixture was stirred at −78° C. for 1 hr and then at rt for 16 hr. The reaction mixture was poured into aq. NH₄Cl saturated solution (30 mL) and extracted with EtOAc (20 mL*2). The combined organic phase was washed with brine (10 mL), dried over anhydrous sodium sulfate, filtered and concentrated under vacuum to give a residue. The residue was purified by prep-HPLC (column: Phenomenex luna C18 150*40 mm*15 μm; mobile phase: [water(FA)-ACN]; gradient:52%-82% B over 15 min) to afford 3-bromo-6-[tert-butoxycarbonyl-[(2,4-dimethoxyphenyl)methyl]amino]-6,7-dihydro-5H-thieno[3,2-b]pyran-2-carboxylic acid (2) (490 mg, 927.31 μmol, 34.74% yield) as a brown solid. ¹H NMR (400 MHz, CHLOROFORM-d) δ=7.18-7.07 (m, 1H), 6.47 (dd, J=2.3, 8.3 Hz, 1H), 6.44 (d, J=2.3 Hz, 1H), 4.49-4.36 (m, 2H), 4.36-4.15 (m, 3H), 3.80 (d, J=13.6 Hz, 6H), 3.32-3.13 (m, 1H), 2.84-2.73 (m, 1H), 1.49 (br s, 9H)

### Procedure for preparation of tert-butyl N-(3-bromo-6,7-dihydro-5H-thieno[3,2-b]pyran-6-yl)-N-[(2,4-dimethoxyphenyl)methyl]carbamate (3)

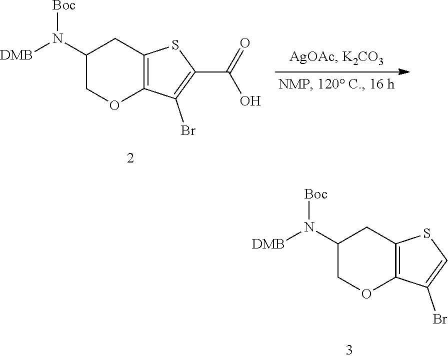

To a solution of 3-bromo-6-[tert-butoxycarbonyl-[(2,4-dimethoxyphenyl)methyl]amino]-6,7-dihydro-5H-thieno[3,2-b]pyran-2-carboxylic acid (2) (490 mg, 927.31 μmol, 1 eq) in NMP (5 mL) was added AgOAc (30.96 mg, 9.50 μL, 0.2 eq) and K₂CO₃ (384.48 mg, 2.78 mmol, 3 eq). The mixture was stirred at 120° C. for 16 hr. The reaction mixture then cooled and poured into H₂O (20 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~20% Ethyl acetate/Petroleum ether gradient @20 mL/min) to afford tert-butyl N-(3-bromo-6,7-dihydro-5H-thieno[3,2-b]pyran-6-yl)-N-[(2,4-dimethoxyphenyl)methyl]carbamate (3) (400 mg, 825.76 μmol, 89.05% yield) as yellow oil. ¹H NMR (400 MHz, CHLOROFORM-d) δ=7.12 (br dd, J=2.0, 5.3 Hz, 1H), 6.95 (s, 1H), 6.46 (dd, J=2.3, 8.3 Hz, 1H), 6.42 (d, J=2.1 Hz, 1H), 4.52-4.07 (m, 5H), 3.79 (d, J=12.3 Hz, 6H), 3.19-3.06 (m, 1H), 2.79-2.69 (m, 1H).

### Procedure for preparation of tert-butyl N-[(2,4-dimethoxyphenyl)methyl]-N-(3-methyl-6,7-dihydro-5H-thieno[3,2-b]pyran-6-yl)carbamate (12)

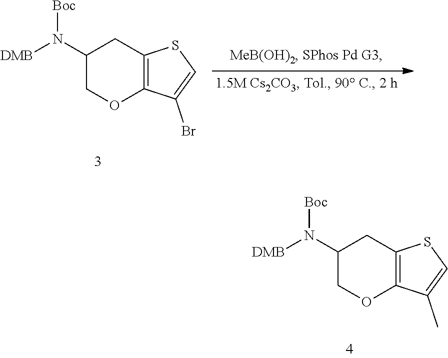

To a solution of tert-butyl N-(3-bromo-6,7-dihydro-5H-thieno[3,2-b]pyran-6-yl)-N-[(2,4-dimethoxyphenyl)methyl]carbamate (3) (200 mg, 412.88 μmol, 1 eq) in Tol. (3 mL) was added methylboronic acid (148.29 mg, 2.48 mmol, 6 eq), SPhos Pd G3 (32.22 mg, 41.29 μmol, 0.1 eq) and aq. Cs₂CO₃ (1.5 M, 825.76 μL, 3 eq). The mixture was stirred at 90° C. for 2 hr then cooled and poured into H₂O (10 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~20% Ethyl acetate/Petroleum ether gradient @20 mL/min) and by prep-HPLC (column: C18 150×30 mm; mobile phase: [water(FA)-ACN]; gradient:70%-100% B over 7 min). Compound tert-butyl N-[(2,4-dimethoxyphenyl)methyl]-N-(3-methyl-6,7-dihydro-5H-thieno[3,2-b]pyran-6-yl)carbamate (4) (110 mg, 262.20 μmol, 63.50% yield) was obtained as yellow oil. ¹H NMR (400 MHz, CHLOROFORM-d) δ=7.12 (td, J=1.8, 3.7 Hz, 1H), 6.59 (s, 1H), 6.49-6.41 (m, 2H), 4.53-4.00 (m, 5H), 3.80 (d, J=13.1 Hz, 6H), 3.05 (dd, J=10.1, 15.4 Hz, 1H), 2.81-2.64 (m, 1H), 2.02 (s, 3H), 1.48 (br s, 9H).

### Procedure for preparation of 3-methyl-6,7-dihydro-5H-thieno[3,2-b]pyran-6-amine hydrochloride (228)

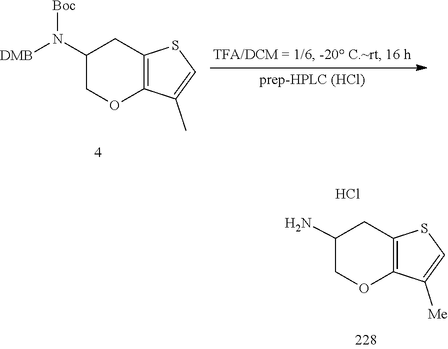

To a solution of tert-butyl N-[(2,4-dimethoxyphenyl)methyl]-N-(3-methyl-6,7-dihydro-5H-thieno[3,2-b]pyran-6-yl)carbamate (4) (110 mg, 262.20 μmol, 1 eq) in DCM (1.2 mL) was added TFA (307.00 mg, 2.69 mmol, 0.2 mL, 10.27 eq) at −20° C. The mixture was slowly warmed up to rt, stirred for 16 hr and concentrated. The residue was purified by prep-HPLC (column: Phenomenex luna C18 150*25 mm*10 μm; mobile phase: [water(HCl)-ACN]; gradient:1%-25% B over 10 min) to afford 3-methyl-6,7-dihydro-5H-thieno[3,2-b]pyran-6-amine hydrochloride (228) (11.50 mg, 55.35 μmol, 21.11% yield) as an off-white solid. ¹H NMR (400 MHz, METHANOL-d4) δ=6.84 (d, J=0.6 Hz, 1H), 4.38-4.30 (m, 1H), 4.18 (dd, J=1.4, 12.0 Hz, 1H), 3.93-3.86 (m, 1H), 3.26 (d, J=5.6 Hz, 1H), 2.85 (br d, J=17.3 Hz, 1H), 2.07 (d, J=1.0 Hz, 3H). LC-MS: m/z [M+H]⁺=170.1

### Synthetic scheme for preparation of 3-cyclopropyl-6,7-dihydro-5H-thieno[3,2-b]pyran-6-amine hydrochloride (229)

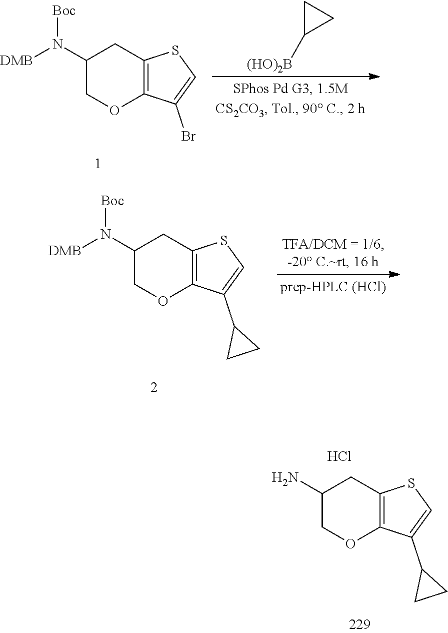

### Procedure for preparation of tert-butyl N-(3-cyclopropyl-6,7-dihydro-5H-thieno[3,2-b]pyran-6-yl)-N-[(2,4-dimethoxyphenyl)methyl]carbamate (2)

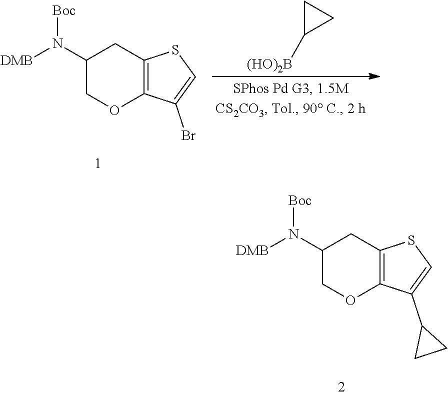

To a solution of tert-butyl N-(3-bromo-6,7-dihydro-5H-thieno[3,2-b]pyran-6-yl)-N-[(2,4-dimethoxyphenyl)methyl]carbamate (1) (200 mg, 412.88 μmol, 1 eq) in Tol. (3 mL) was added cyclopropyl boronic acid (177.33 mg, 2.06 mmol, 5 eq), SPhos Pd G3 (32.22 mg, 41.29 μmol, 0.1 eq) and aq. Cs₂CO₃ (1.5 M, 825.76 μL, 3 eq). The mixture was stirred at 90° C. for 2 hr then cooled and poured into H₂O (20 mL) and extracted with EtOAc (20 mL*2). The combined organic phases were washed with brine (10 mL), dried over anhydrous sodium sulfate, filtered and concentrated under vacuum. The residue was purified by flash silica gel chromatography (ISCO®; 4 g SepaFlash® Silica Flash Column, eluent 0~20% Ethyl acetate/Petroleum ether gradient @20 mL/min) and then by prep-HPLC (column: C18 150×30 mm; mobile phase: [water(FA)-ACN]; gradient:70%-100% B over 7 min). Compound tert-butyl N-(3-cyclopropyl-6,7-dihydro-5H-thieno[3,2-b]pyran-6-yl)-N-[(2,4-dimethoxyphenyl)methyl]carbamate (2) (120 mg, 269.32 μmol, 65.23% yield) was obtained as yellow oil. ¹H NMR (400 MHz, CHLOROFORM-d) δ=7.17-7.07 (m, 1H), 6.47 (dd, J=2.4, 8.4 Hz, 1H), 6.44-6.37 (m, 2H), 4.52-4.04 (m, 5H), 3.80 (d, J=12.4 Hz, 6H), 3.05 (dd, J=10.4, 15.2 Hz, 1H), 2.79-2.65 (m, 1H), 1.75-1.67 (m, 1H), 1.48 (br s, 9H), 0.86-0.75 (m, 2H), 0.67-0.55 (m, 2H).

### Procedure for preparation of 3-cyclopropyl-6,7-dihydro-5H-thieno[3,2-b]pyran-6-amine hydrochloride (229)

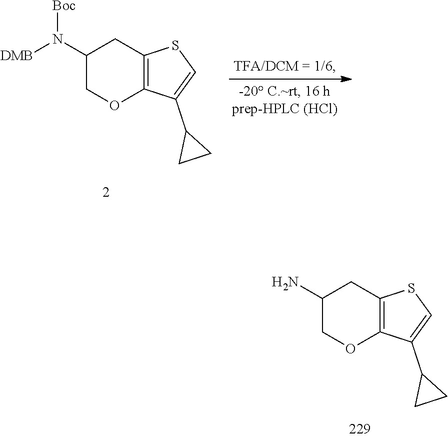

To a solution of tert-butyl N-(3-cyclopropyl-6,7-dihydro-5H-thieno[3,2-b]pyran-6-yl)-N-[(2,4-dimethoxyphenyl)methyl]carbamate (2) (120 mg, 269.32 μmol, 1 eq) in DCM (1.2 mL) was added TFA (307.00 mg, 2.69 mmol, 0.2 mL, 10.00 eq) at −20° C. The mixture was slowly warmed up to 25° C. and stirred for 16 hr and then concentrated. The residue was purified by prep-HPLC (column: Phenomenex luna C18 150*25 mm*10 μm; mobile phase: [water(HCl)-ACN]; gradient:1%-30% B over 10 min) to afford 3-cyclopropyl-6,7-dihydro-5H-thieno[3,2-b]pyran-6-amine hydrochloride (229) (18.01 mg, 76.94 μmol, 28.57% yield) as an off-white solid. 1H NMR (400 MHz, METHANOL-d4) δ=6.66 (s, 1H), 4.38-4.31 (m, 1H), 4.20 (dd, J=1.0, 11.9 Hz, 1H), 3.90 (br s, 1H), 3.29-3.20 (m, 1H), 2.83 (br d, J=17.3 Hz, 1H), 1.79 (tt, J=5.1, 8.5 Hz, 1H), 0.87-0.78 (m, 2H), 0.72-0.60 (m, 2H). LC-MS: m/z [M+H]⁺=196.2

### Synthetic scheme for preparation of N-methyl-3-(trifluoromethyl)-4,5,6,7-tetrahydrobenzothiophen-6-amine hydrochloride enantiomer 1 (230) and enantiomer 2 (231)

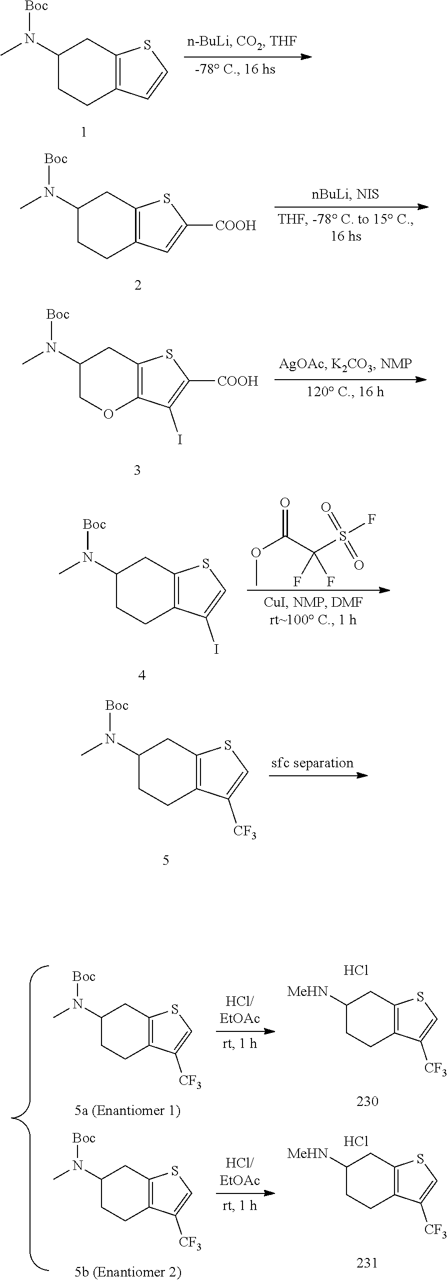

### Procedure for preparation of 6-[tert-butoxycarbonyl(methyl)amino]-4,5,6,7-tetrahydrobenzothiophene-2-carboxylic acid (2)

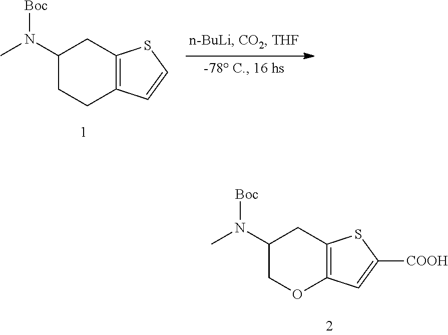 [SMILES: CN(C(=O)OC(C)(C)C)C1CCc2ccsc2C1 ; CN(C(=O)OC(C)(C)C)C1COc2cc(C(=O)O)sc2C1]

To a mixture of tert-butyl N-methyl-N-(4,5,6,7-tetrahydrobenzothiophen-6-yl) carbamate (1) (5 g, 18.70 mmol, 1 eq) in THF (40 mL) was added n-BuLi (2.5 M, 8.98 mL, 1.2 eq) at −78° C. under N₂. The mixture was stirred at −78° C. for 0.5 h. Then CO₂ (822.96 mg, 18.70 mmol, 1 eq) was bubbled into the mixture and the reaction vessel was purged with CO₂ (822.96 mg, 18.70 mmol, 1 eq) several times. The mixture was stirred under CO₂ (822.96 mg, 18.70 mmol, 1 eq) at −78° C. for 15.5 h. and then was quenched by addition of aq. sat.NH₄Cl (40 mL) at 0° C. Followed dilution with H₂O (30 mL) and extraction with EtOAc (60 mL*3). The combined organic layers were washed with aq. sat.NaCl (40 mL), dried over anhydrous Na₂SO₄, filtered and concentrated under reduced pressure. The residue was purified by flash silica gel chromatography (ISCO®; 40 g SepaFlash® Silica Flash Column, eluent 0~30% Ethyl acetate/Petroleum ether gradient @50 mL/min). Compound 6-[tert-butoxycarbonyl(methyl)amino]-4,5,6,7-tetrahydrobenzothiophene-2-carboxylic acid (2) (2.8 g, 8.22 mmol, 43.94% yield) was obtained as a yellow solid. LC-MS: m/z [M+H-tBu]⁺=256.0.

### Procedure for preparation of 6-[tert-butoxycarbonyl(methyl)amino]-3-iodo-4,5,6,7-tetrahydrobenzothiophene-2-carboxylic acid (3)

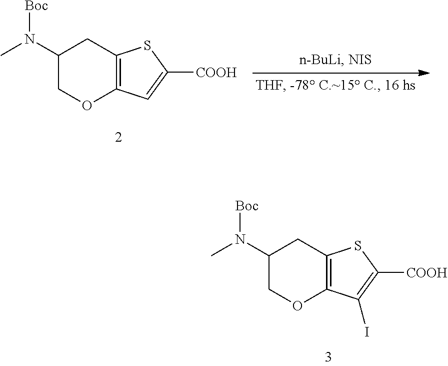 [SMILES: CN(C(=O)OC(C)(C)C)C1COc2cc(C(=O)O)sc2C1 ; CN(C(=O)OC(C)(C)C)C1COc2c(sc(C(=O)O)c2I)C1]

To a solution of 6-[tert-butoxycarbonyl(methyl)amino]-4,5,6,7-tetrahydrobenzothiophene-2-carboxylic acid (2) (2.8 g, 8.99 mmol, 1 eq) in THF (30 mL) was added n-BuLi (2.5 M, 8.63 mL, 2.4 eq) at −78° C. under N₂ atmosphere. After stirring for 1 h, a solution of NIS (3.64 g, 16.19 mmol, 1.8 eq) in THF (15 mL) was added drop-wise at −70° C. and the resulting mixture was stirred for 1 h at −78° C., Then the mixture was warm to −15° C. slowly and stirred for 16 h. The reaction mixture was quenched by addition aq. sat. NH₄Cl 40 mL at 0° C., and then diluted with H₂O (40 mL) and extracted with EtOAc (60 mL*3). The combined organic layers were washed with aq. sat. NaCl (40 mL), dried over anhydrous Na₂SO₄, filtered and concentrated under reduced pressure to give a residue. The residue was purified by flash silica gel chromatography (ISCO®; 20 g SepaFlash® Silica Flash Column, eluent 0~30% Ethyl acetate/Petroleum ether gradient @60 mL/min). Compound 6-[tert-butoxycarbonyl (methyl)amino]-3-iodo-4,5,6,7-tetrahydrobenzothiophene-2-carboxylic acid (3) (2.55 g, 4.37 mmol, 48.64% yield) was obtained as a yellow solid. LC-MS: m/z [M+H-tBu]⁺=381.9.

### Procedure for preparation of tert-butyl N-(3-iodo-4,5,6,7-tetrahydrobenzothiophen-6-yl)-N-methyl-carbamate (4)

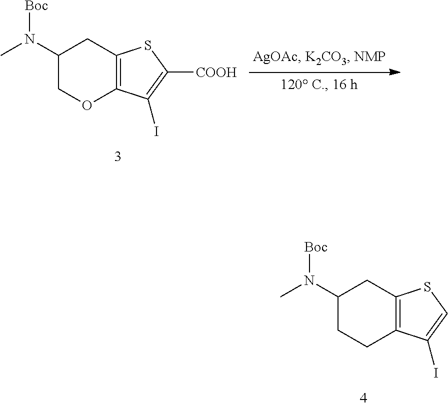 [SMILES: CN(C(=O)OC(C)(C)C)C1COc2c(sc(C(=O)O)c2I)C1 ; CN(C(=O)OC(C)(C)C)C1CCc2c(I)csc2C1]

To a solution of 6-[tert-butoxycarbonyl(methyl)amino]-3-iodo-4,5,6,7-tetrahydrobenzothiophene-2-carboxylic acid (3) (2.45 g, 5.60 mmol, 1 eq) in NMP (25 mL) was added AgOAc (280.55 mg, 1.68 mmol, 0.3 eq) and K₂CO₃ (2.32 g, 16.81 mmol, 3 eq). The mixture was stirred at 120° C. for 16 hr. The reaction mixture was diluted with EtOAc (20 mL) and filtered through a pad of diatomite earth. To the filtrate was added H₂O (30 mL) and the mixture was extracted with EtOAc (10 mL*3). The combined organic layers were washed with brine (10 mL*3), dried over anhydrous sodium sulfate, filtered and concentrated under reduced pressure. The residue was purified by flash silica gel chromatography (ISCO®; 12 g SepaFlash® Silica Flash Column, eluent 0~10% Ethyl acetate/Petroleum ether gradient @36 mL/min). Compound tert-butyl N-(3-iodo-4,5,6,7-tetrahydrobenzothiophen-6-yl)-N-methyl-carbamate (4) (1.35 g, 3.22 mmol, 57.47% yield) was obtained as a white solid. LC-MS: m/z [M+H-tBu]⁺=337.9.

### Procedure for preparation of tert-butyl N-methyl-N-[3-(trifluoromethyl)-4,5,6,7-tetrahydrobenzothiophen-6-yl]carbamate enantiomer 1 (5a) and enantiomer 2 (5b)

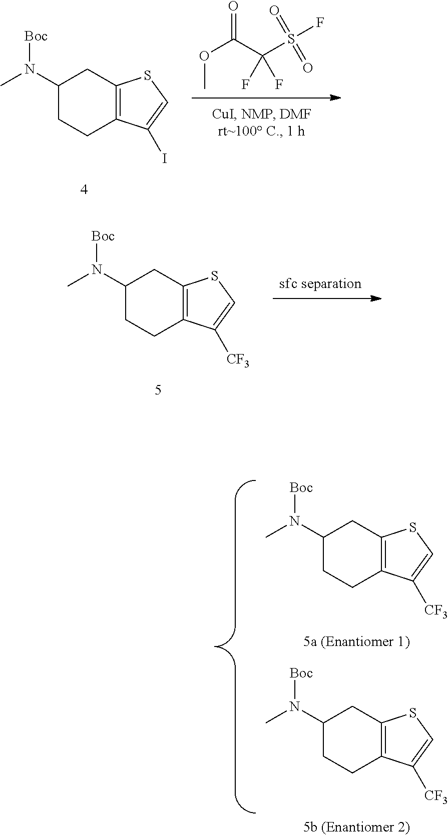

To a solution of tert-butyl N-(3-iodo-4,5,6,7-tetrahydrobenzothiophen-6-yl)-N-methyl-carbamate (4) (500 mg, 1.27 mmol, 1 eq) in DMF (5 mL) was added methyl 2,2-difluoro-2-fluorosulfonyl-acetate (1.22 g, 6.36 mmol, 808.76 μL, 5 eq), CuI (1.21 g, 6.36 mmol, 5 eq) and NMP (189.05 mg, 1.91 mmol, 185.34 μL, 1.5 eq). The mixture was stirred at 100° C. for 1 h then cooled and poured into H₂O (10 mL). The mixture was diluted with NH₃·H₂O (5 mL) and extracted with EtOAc (5 mL*3). The combined organic layers were washed with brine (5 mL*3), dried over anhydrous sodium sulfate, filtered and concentrated under reduced pressure to give a residue. The residue was purified by flash silica gel chromatography (ISCO®; 12 g SepaFlash® Silica Flash Column, eluent 0~12% Ethyl acetate/Petroleum ether gradient @36 mL/min) and by prep-HPLC (column: Waters Xbridge Prep OBD C18 150*40 mm*10 μm; mobile phase: [water(NH₄HCO₃)-ACN]; gradient:55%-85% B over 15 min) to afford the racemic product (0.3 g).

The racemic material was separated to its enantiomers by SFC (column: DAICEL CHIRALPAK IG (250 mm*50 mm, 10 μm); mobile phase: [CO₂-MeOH (0.1% NH₃H₂O)]; B %:10%, isocratic elution mode).and. Compound tert-butyl N-methyl-N-[3-(trifluoromethyl)-4,5,6,7-tetrahydrobenzothiophen-6-yl]carbamate enantiomer 1 (5a) (1st peak in SFC, Rt=0.590 min) (80 mg, 233.76 μmol, 21.15% yield) obtained as a white solid. LC-MS: m/z [M+H-tBu]⁺=280.0. Compound tert-butyl N-methyl-N-[3-(trifluoromethyl)-4,5,6,7-tetrahydrobenzothiophen-6-yl]carbamate enantiomer 2 (5b) (2nd peak in SFC, Rt=0.726 min) (45 mg, 128.81 μmol, 11.66% yield) was obtained as a white solid. LC-MS: m/z [M+H-tBu]⁺=280.0.

### Procedure for preparation of N-methyl-3-(trifluoromethyl)-4,5,6,7-tetrahydrobenzothiophen-6-amine hydrochloride enantiomer 1 (230)

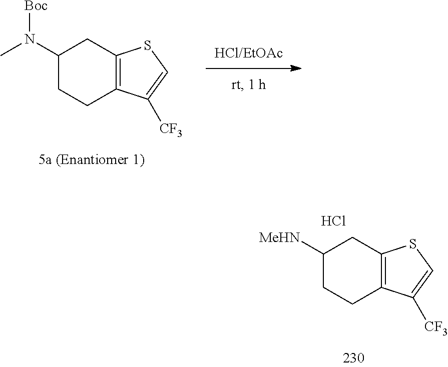 [SMILES: CN(C(=O)OC(C)(C)C)C1CCc2c(C(F)(F)F)csc2C1 ; Cl ; CNC1CCc2c(C(F)(F)F)csc2C1]

To a solution of tert-butyl N-methyl-N-[3-(trifluoromethyl)-4,5,6,7-tetrahydrobenzothiophen-6-yl]carbamate (5a) (80 mg, 238.53 μmol, 1 eq) in EtOAc (1 mL) was added HCl/EtOAc (4 M, 5.33 mL, 89.44 eq). The mixture was stirred at rt for 1 hr andconcentrated under vacuum to give the product. Compound N-methyl-3-(trifluoromethyl)-4,5,6,7-tetrahydrobenzothiophen-6-amine hydrochloride enantiomer 1 (230) (39.74 mg, 167.83 μmol, 70.36% yield) was obtained as a white solid. ¹H NMR (400 MHz, METHANOL-d₄) δ=7.85 (s, 1H), 3.66-3.54 (m, 1H), 3.36 (br dd, J=5.4, 16.3 Hz, 2H), 3.01-2.74 (m, 6H), 2.38-2.26 (m, 1H), 2.04-1.89 (m, 1H). LC-MS: m/z [M+H]⁺=236.1.

### Procedure for preparation of N-methyl-3-(trifluoromethyl)-4,5,6,7-tetrahydrobenzothiophen-6-amine hydrochloride enantiomer 2 (231)

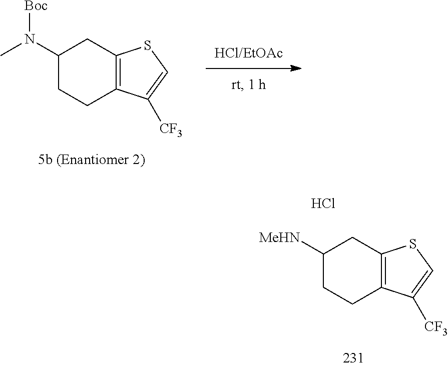 [SMILES: CN(C(=O)OC(C)(C)C)C1CCc2c(C(F)(F)F)csc2C1 ; Cl ; CNC1CCc2c(C(F)(F)F)csc2C1]

To a solution of tert-butyl N-methyl-N-[3-(trifluoromethyl)-4,5,6,7-tetrahydrobenzothiophen-6-yl]carbamate enantiomer 2 (5b) (45 mg, 134.17 μmol, 1 eq) in EtOAc (1 mL) was added HCl/EtOAc (4 M, 3.00 mL, 89.44 eq). The mixture was stirred at rt for 1 hr and then concentrated under vacuum to give the product. Compound N-methyl-3-(trifluoromethyl)-4,5,6,7-tetrahydrobenzothiophen-6-amine hydrochloride enantiomer 2 (231) (23.09 mg, 97.19 μmol, 72.44% yield) was obtained as a white solid. ¹H NMR (400 MHz, METHANOL-d₄) δ=7.85 (s, 1H), 3.67-3.52 (m, 1H), 3.44-3.34 (m, 2H), 3.03-2.73 (m, 6H), 2.41-2.23 (m, 1H), 1.95 (dtd, J=5.9, 10.2, 12.9 Hz, 1H)). LC-MS: m/z [M+H]⁺=236.0.

### Synthetic Scheme for preparation of N-methyl-3-phenyl-4,5,6,7-tetrahydrobenzothiophen-5-amine hydrochloride enantiomer 1 (232) and enantiomer2 (233)

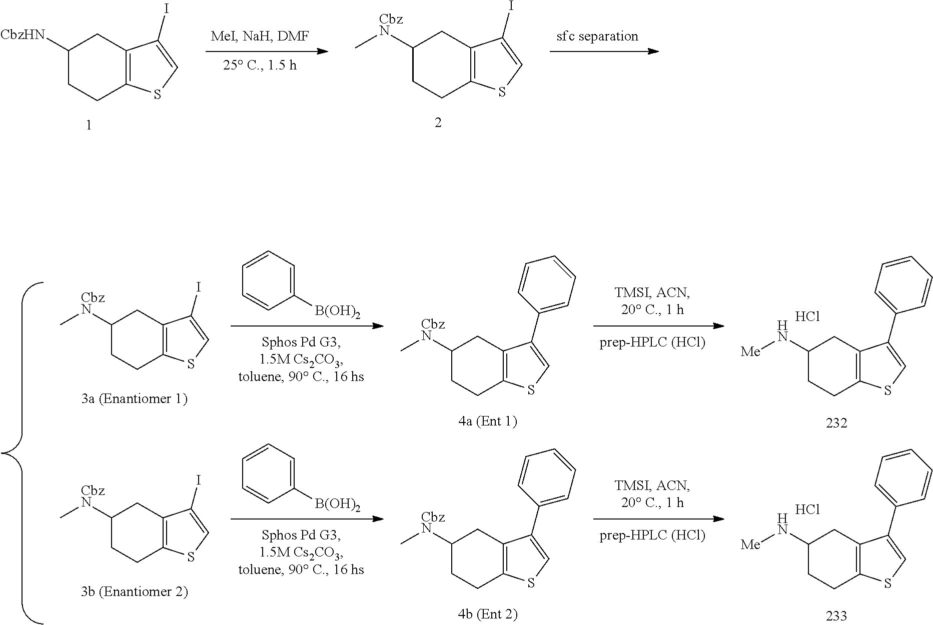

### Procedure for preparation of benzyl N-(3-iodo-4,5,6,7-tetrahydrobenzothiophen-5-yl)-N-methyl-carbamate (2)

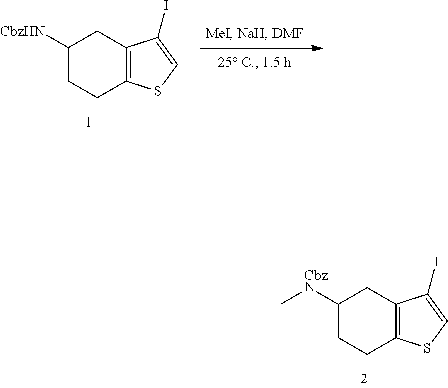 [SMILES: O=C(NC1CCc2scc(I)c2C1)OCc1ccccc1 ; CN(C(=O)OCc1ccccc1)C1CCc2scc(I)c2C1]

To a solution of NaH (69.68 mg, 1.74 mmol, 60% suspension in mineral oil, 1.2 eq) in DMF (6 mL) was added benzyl N-(3-iodo-4,5,6,7-tetrahydrobenzothiophen-5-yl)carbamate (1) (600 mg, 1.45 mmol, 1 eq). The mixture was stirred at 0° C. for 0.5 h, then MeI (309.10 mg, 2.18 mmol, 135.57 μL, 1.5 eq) was added and mixture the mixture was warmed up to 25° C. and stirred at this temperature for 1 h. The reaction mixture was quenched by aq. sat. NH₄Cl (20 mL), diluted with water (20 mL), and extracted with EtOAc (20 mL*3). The combined organic phases were washed with brine (30 mL), dried over anhydrous sodium sulfate, filtered and concentrated under vacuum to give a residue. The residue was purified by flash silica gel chromatography (ISCO®; 20 g SepaFlash® Silica Flash Column, eluent 0~20% Ethyl acetate/Petroleum ether gradient @50 mL/min). Compound benzyl N-(3-iodo-4,5,6,7-tetrahydrobenzothiophen-5-yl)-N-methyl-carbamate (2) (490 mg, 1.15 mmol, 78.99% yield) was obtained as a white solid. LCMS: m/z [M+H]⁺=328.1.

### Procedure for preparation of benzyl N-(3-iodo-4,5,6,7-tetrahydrobenzothiophen-5-yl)-N-methyl-carbamate enantiomer 1 (3a) and enantiomer 2 (3b)

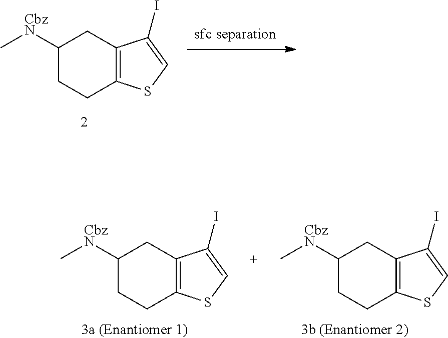 [SMILES: CN(C(=O)OCc1ccccc1)C1CCc2scc(I)c2C1 ; CN(C(=O)OCc1ccccc1)C1CCc2scc(I)c2C1 ; CN(C(=O)OCc1ccccc1)C1CCc2scc(I)c2C1]

Racemic benzyl N-(3-iodo-4,5,6,7-tetrahydrobenzothiophen-5-yl)-N-methyl-carbamate (2) (490 mg) was separated by SFC (column: DAICEL CHIRALPAK AD (250 mm*30 mm, 10 μm); mobile phase: [CO₂-i-PrOH (0.1% NH₃H₂O)]; B %:30%, isocratic elution mode) to afford benzyl N-(3-iodo-4,5,6,7-tetrahydrobenzothiophen-5-yl)-N-methyl-carbamate enantiomer 1 (3a) (258 mg, 603.79 μmol, 51.60% yield) as a white solid and benzyl N-(3-iodo-4,5,6,7-tetrahydrobenzothiophen-5-yl)-N-methyl-carbamate enantiomer 2 (3b) (132 mg, 308.92 μmol, 26.40% yield) a white solid.

### Procedure for preparation of benzyl N-methyl-N-(3-phenyl-4,5,6,7-tetrahydrobenzothiophen-5-yl)carbamate enantiomer 1 (4a)

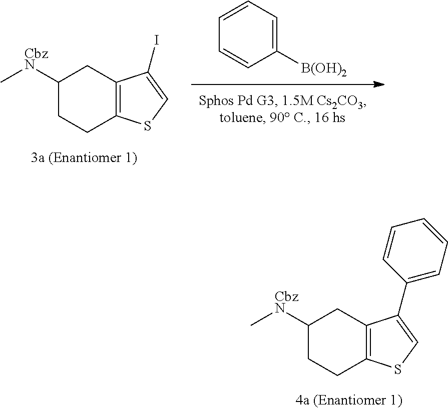 [SMILES: CN(C(=O)OCc1ccccc1)C1CCc2scc(I)c2C1 ; OB(O)c1ccccc1 ; CN(C(=O)OCc1ccccc1)C1CCc2scc(-c3ccccc3)c2C1]

To a solution of benzyl N-(3-iodo-4,5,6,7-tetrahydrobenzothiophen-5-yl)-N-methyl-carbamate enantiomer 1 (3a) (100 mg, 234.03 μmol, 1 eq) in Tol. (1 mL) was added phenylboronic acid (142.67 mg, 1.17 mmol, 5 eq), Sphos Pd G3 (18.26 mg, 23.40 μmol, 0.1 eq), Cs₂CO₃ (1.5 M aq., 468.06 μL, 3 eq) and then the mixture was purged with N₂ for 3 times. The mixture was stirred at 90° C. for 16 hr under N₂ atmosphere. The reaction mixture was concentrated under reduced pressure to remove toluene. The residue was diluted with water (10 mL) and extracted with EtOAc (5 mL*3). The combined organic layers were washed with brine (5 mL*2), dried over Na₂SO₄, filtered and concentrated to give a residue. The residue was purified by column chromatography (SiO2, Petroleum ether/Ethyl acetate=5/1). Compound benzyl N-methyl-N-(3-phenyl-4,5,6,7-tetrahydrobenzothiophen-5-yl)carbamate enantiomer 1 (4a) (40 mg, 105.96 μmol, 45.28% yield) was obtained as a white solid. LCMS: m/z [M+H]⁺=378.

### Procedure for preparation of benzyl N-methyl-N-(3-phenyl-4,5,6,7-tetrahydrobenzothiophen-5-yl)carbamate enantiomer 2 (4b)

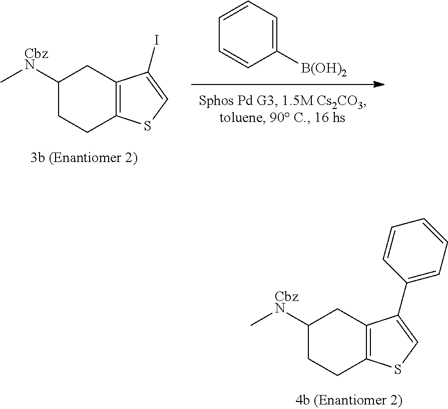 [SMILES: CN(C(=O)OCc1ccccc1)C1CCc2scc(I)c2C1 ; OB(O)c1ccccc1 ; CN(C(=O)OCc1ccccc1)C1CCc2scc(-c3ccccc3)c2C1]

To a solution of benzyl N-(3-iodo-4,5,6,7-tetrahydrobenzothiophen-5-yl)-N-methyl-carbamate enantiomer 2 (3b) (132 mg, 308.92 μmol, 1 eq) in Tol. (2 mL) was added phenylboronic acid (188.33 mg, 1.54 mmol, 5 eq), Sphos Pd G3 (24.10 mg, 30.89 μmol, 0.1 eq) Cs₂CO₃ (1.5 M aq., 617.83 μL, 3 eq) and then the mixture was purged with N₂ for 3 times. The mixture was stirred at 90° C. for 16 hr under N₂ atmosphere. The reaction mixture was concentrated under reduced pressure to remove toluene. The residue was diluted with water (10 mL) and extracted with EtOAc (5 mL*3). The combined organic layers were washed with brine (5 mL*2), dried over anhydrous sodium sulfate, filtered and concentrated under reduced pressure to give a residue. The residue was purified by column chromatography (SiO2, Petroleum ether/Ethyl acetate=5/1). Compound benzyl N-methyl-N-(3-phenyl-4,5,6,7-tetrahydrobenzothiophen-5-yl)carbamate enantiomer 2 (4b) (70 mg, 185.43 μmol, 60.03% yield) was obtained as a white solid. LCMS: m/z [M+H]⁺=378.

### Procedure for preparation of N-methyl-3-phenyl-4,5,6,7-tetrahydrobenzothiophen-5-amine hydrochloride enantiomer 1 (232)

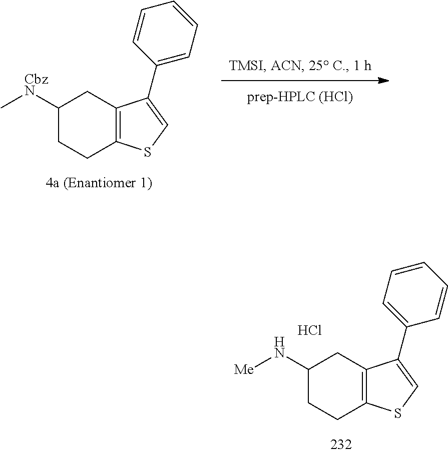 [SMILES: CN(C(=O)OCc1ccccc1)C1CCc2scc(-c3ccccc3)c2C1 ; CNC1CCc2scc(-c3ccccc3)c2C1 ; Cl]

To a solution of benzyl N-methyl-N-(3-phenyl-4,5,6,7-tetrahydrobenzothiophen-5-yl)carbamate enantiomer 1 (4a) (40 mg, 105.96 μmol, 1 eq) in ACN (1 mL) was added iodo(trimethyl)silane (63.61 mg, 317.88 μmol, 43.27 μL, 3 eq). The mixture was stirred at 25° C. for 1 h. The reaction mixture was concentrated under reduced pressure to remove ACN. The residue was purified by prep-HPLC (HCl condition:column: Phenomenex luna C18 150*25 mm*10 μm; mobile phase: [water(HCl)-ACN]; gradient:10%-40% B over 10 min). Compound N-methyl-3-phenyl-4,5,6,7-tetrahydrobenzothiophen-5-amine hydrochloride enantiomer 1 (232) (6.53 mg, 26.83 μmol, 25.32% yield) was obtained as a white solid. ¹H NMR (400 MHz, METHANOL-d₄) δ=7.49-7.31 (m, 5H), 7.29-7.19 (m, 1H), 3.57-3.48 (m, 1H), 3.19-2.99 (m, 3H), 2.86-2.68 (m, 4H), 2.46-2.35 (m, 1H), 2.14-2.02 (m, 1H). LCMS: m/z [M+H]⁺=244.2

### Procedure for preparation of N-methyl-3-phenyl-4,5,6,7-tetrahydrobenzothiophen-5-amine hydrochloride enantiomer 2 (233)

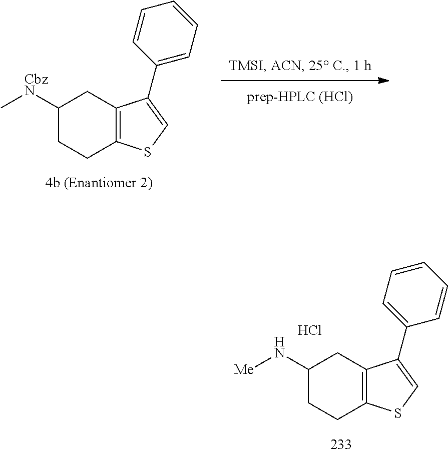 [SMILES: CN(C(=O)OCc1ccccc1)C1CCc2scc(-c3ccccc3)c2C1 ; CNC1CCc2scc(-c3ccccc3)c2C1 ; Cl]

To a solution of benzyl N-methyl-N-(3-phenyl-4,5,6,7-tetrahydrobenzothiophen-5-yl)carbamate enantiomer 2 (4b) (70 mg, 185.43 μmol, 1 eq) in ACN (1 mL) was added iodo(trimethyl)silane (95.41 mg, 476.82 μmol, 64.90 μL, 3 eq). The mixture was stirred at 25° C. for 1 h. The reaction mixture was concentrated under reduced pressure to remove ACN. The residue was purified by prep-HPLC (HCl condition:column: Phenomenex luna C18 150*25 mm*10 μm; mobile phase: [water(HCl)-ACN]; gradient:10%-40% B over 10 min). Compound N-methyl-3-phenyl-4,5,6,7-tetrahydrobenzothiophen-5-amine hydrochloride enantiomer 2 (233) (48.26 mg, 172.46 μmol, 93.01% yield) was obtained as a white solid. ¹H NMR (400 MHz, METHANOL-d₄) δ=7.46-7.28 (m, 5H), 7.22 (s, 1H), 3.55-3.43 (m, 1H), 3.14-2.96 (m, 3H), 2.80-2.71 (m, 4H), 2.42-2.34 (m, 1H), 2.11-1.96 (m, 1H). LCMS: m/z [M+H]⁺=244.2

### Synthetic scheme for preparation of N-methyl-3,4-dihydro-2H-thieno[2,3-b]pyran-3-amine hydrochloride (234)

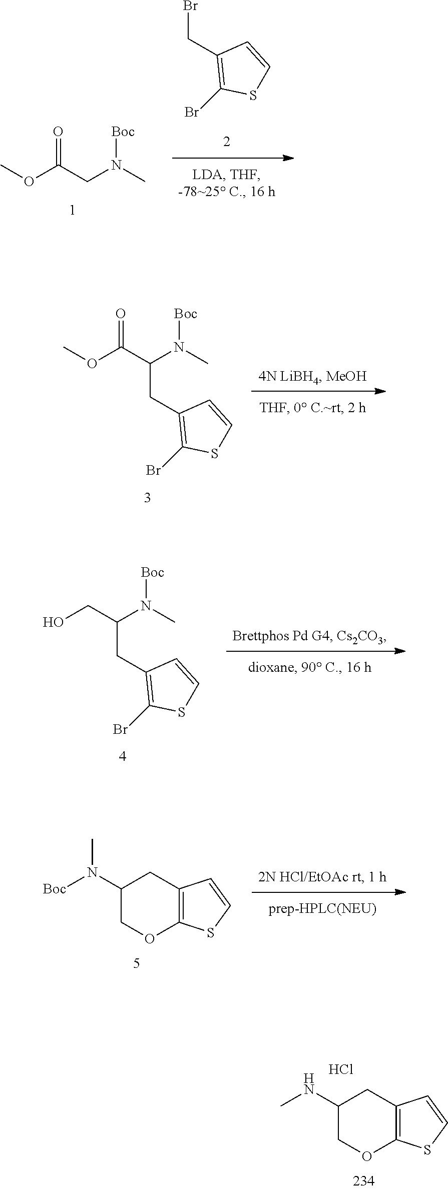

### Procedure for preparation of methyl 3-(2-bromo-3-thienyl)-2-[tert-butoxycarbonyl(methyl)amino]propanoate (3)

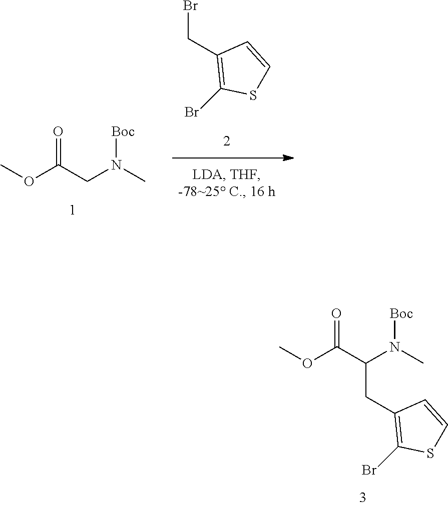

To a solution of LDA (2 M, 49.20 mL, 1.5 eq) in THF (300 mL) was added methyl 2-[tert-butoxycarbonyl(methyl)amino]acetate (1) (20 g, 98.41 mmol, 1.5 eq) dropwise at −78° C., the mixture was stirred for 1 hr, and then 2-bromo-3-(bromomethyl)thiophene (16.79 g, 65.61 mmol, 1 eq) was added at −78° C. The resulting mixture was stirred at −78° C. for 1 h and then at 25° C. for 15 hr under N₂. The reaction mixture was quenched with aq. sat.NH₄Cl (300 mL) and the mixture was extracted with EtOAc (100 mL*2). The combined organic layers were washed with aq. sat.NaCl (50 mL), dried over anhydrous Na₂SO₄, filtered and concentrated under reduced pressure. The residue was purified by flash silica gel chromatography (ISCO®; 120 g SepaFlash® Silica Flash Column, eluent 0~12% Ethyl acetate/Petroleum ether gradient @60 mL/min). Compound methyl 3-(2-bromo-3-thienyl)-2-[tert-butoxycarbonyl(methyl)amino]propanoate (3) (20 g, 52.87 mmol, 80.59% yield) was obtained as a colorless oil. 1H NMR (400 MHz, CHLOROFORM-d) δ=7.21 (br d, J=5.6 Hz, 1H), 6.93-6.75 (m, 1H), 4.98-4.44 (m, 1H), 3.77 (br s, 3H), 3.32-3.03 (m, 2H), 2.86-2.65 (m, 3H), 1.47-1.37 (m, 9H). LC-MS: m/z [M+H]⁺=380.0

### Procedure for preparation of tert-butyl N-[1-[(2-bromo-3-thienyl)methyl]-2-hydroxy-ethyl]-N-methyl-carbamate (4)

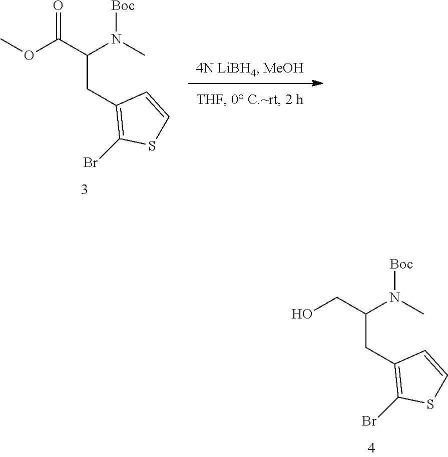 [SMILES: COC(=O)C(Cc1ccsc1Br)N(C)C(=O)OC(C)(C)C ; CN(C(=O)OC(C)(C)C)C(CO)Cc1ccsc1Br]

To a solution of methyl 3-(2-bromo-3-thienyl)-2-[tert-butoxycarbonyl(methyl)amino]propanoate (3) (20 g, 52.87 mmol, 1 eq) in THF (200 mL) was added LiBH₄ (4 M in THF, 66.09 mL, 5 eq), then it was added MeOH (5 mL) at 0° C. Then mixture was allowed to warm up naturally and slowly to 25° C. and stirred for 2 hr under N₂. The reaction mixture was the diluted with aq, sat.NH₄Cl 1000 mL and extracted with EtOAc (100 mL*2). The combined organic layers were washed with aq. sat. NaCl (100 mL), dried over anhydrous Na₂SO₄, filtered and concentrated under reduced pressure. The residue was purified by flash silica gel chromatography (ISCO®; 80 g SepaFlash® Silica Flash Column, eluent 0~35% Ethyl acetate/Petroleum ether gradient @50 mL/min). Compound tert-butyl N-[1-[(2-bromo-3-thienyl)methyl]-2-hydroxy-ethyl]-N-methyl-carbamate (4) (18.3 g, 52.25 mmol, 98.82% yield) was obtained as a yellow oil. ¹H NMR (400 MHz, CHLOROFORM-d) δ=7.20 (d, J=5.6 Hz, 1H), 6.94-6.71 (m, 1H), 4.43-4.10 (m, 1H), 3.73 (br s, 2H), 2.99-2.64 (m, 6H), 1.40 (br d, J=17.9 Hz, 9H). LC-MS: m/z [M+H-tBu]⁺=294.0

### Procedure for preparation of tert-butyl N-(3,4-dihydro-2H-thieno[2,3-b]pyran-3-yl)-N-methyl-carbamate (5)

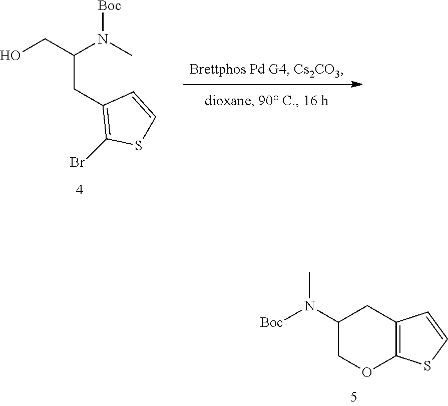 [SMILES: CN(C(=O)OC(C)(C)C)C(CO)Cc1ccsc1Br ; CN(C(=O)OC(C)(C)C)C1COc2sccc2C1]

A mixture of tert-butyl N-[1-[(2-bromo-3-thienyl)methyl]-2-hydroxy-ethyl]-N-methyl-carbamate (4) (3 g, 8.56 mmol, 1 eq), Brettphos Pd G4 (788 mg, 856 μmol, 0.1 eq) and Cs₂CO₃ (8.37 g, 25.6 mmol, 3 eq) in dioxane (250 mL) was degassed and purged with N₂ for 3 times, and then the mixture was stirred at 90° C. for 16 hr under N₂ atmosphere. The reaction mixture was diluted with H₂O (300 mL) and extracted with EtOAc (200 mL*2). The combined organic layers were washed with aq, sat.NaCl (100 mL), dried over anhydrous Na₂SO₄, filtered and concentrated under reduced pressure to give a residue. The residue was purified by flash silica gel chromatography (ISCO®; 120 g SepaFlash® Silica Flash Column, eluent 0~12% Ethyl acetate/Petroleum ether gradient @50 mL/min). Compound tert-butyl N-(3,4-dihydro-2H-thieno[2,3-b]pyran-3-yl)-N-methyl-carbamate (5) (460 mg, 1.71 mmol) was obtained as a yellow oil. ¹H NMR (400 MHz, CHLOROFORM-d) δ=6.57 (d, J=5.7 Hz, 1H), 6.48 (d, J=5.7 Hz, 1H), 4.67-4.34 (m, 1H), 4.25-4.08 (m, 2H), 2.90-2.71 (m, 5H), 1.49 (s, 9H). LC-MS: m/z [M+H-Bu]⁺=214.1

### Procedure for preparation of N-methyl-3,4-dihydro-2H-thieno[2,3-b]pyran-3-amine hydrochloride (234)

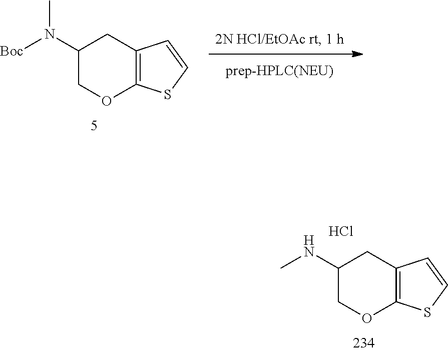 [SMILES: CN(C(=O)OC(C)(C)C)C1COc2sccc2C1 ; Cl ; CNC1COc2sccc2C1]

To a solution of tert-butyl N-(3,4-dihydro-2H-thieno[2,3-b]pyran-3-yl)-N-methyl-carbamate (100 mg, 371.25 μmol, 1 eq) in EtOAc (2 mL) was added HCl/EtOAc (4 M, 2 mL, 21.55 eq). The mixture was stirred at 25° C. for 1 hr. The mixture was concentrated under reduced pressure to give a residue. The residue was treated with aq, sat. NaHCO₃to pH 8, and then purified by prep-HPLC (column: Waters Xbridge 150*25 mm*5 μm; mobile phase: [water(NH₄HCO₃)-ACN]; gradient:14%-44% B over 9 min). Compound N-methyl-3,4-dihydro-2H-thieno[2,3-b]pyran-3-amine hydrochloride (234) (19.25 mg, 92.05 μmol, 24.79% yield) was obtained as an orange solid. ¹H NMR (400 MHz, METHANOL-d4) δ=6.66 (d, J=0.9 Hz, 2H), 4.58-4.49 (m, 1H), 4.28 (d, J=12.1 Hz, 1H), 3.74 (br d, J=2.3 Hz, 1H), 3.15 (dd, J=5.8, 17.4 Hz, 1H), 2.90-2.76 (m, 4H). ¹H NMR (400 MHz, DMSO-d6) δ=9.70-8.37 (m, 2H), 6.74 (d, J=5.8 Hz, 1H), 6.67 (d, J=5.8 Hz, 1H), 4.45-4.37 (m, 1H), 4.36-4.29 (m, 1H), 3.65 (dq, J=2.4, 5.3 Hz, 1H), 3.01 (dd, J=5.8, 16.6 Hz, 1H), 2.81 (dd, J=4.6, 16.6 Hz, 1H), 2.61 (s, 3H). LC-MS: m/z [M+H]⁺=170.2

### Synthetic scheme for the synthesis of 3-cyclopropyl-N-methyl-6,7-dihydro-5H-thieno[3,2-b]pyran-6-amine hydrochloride (235)

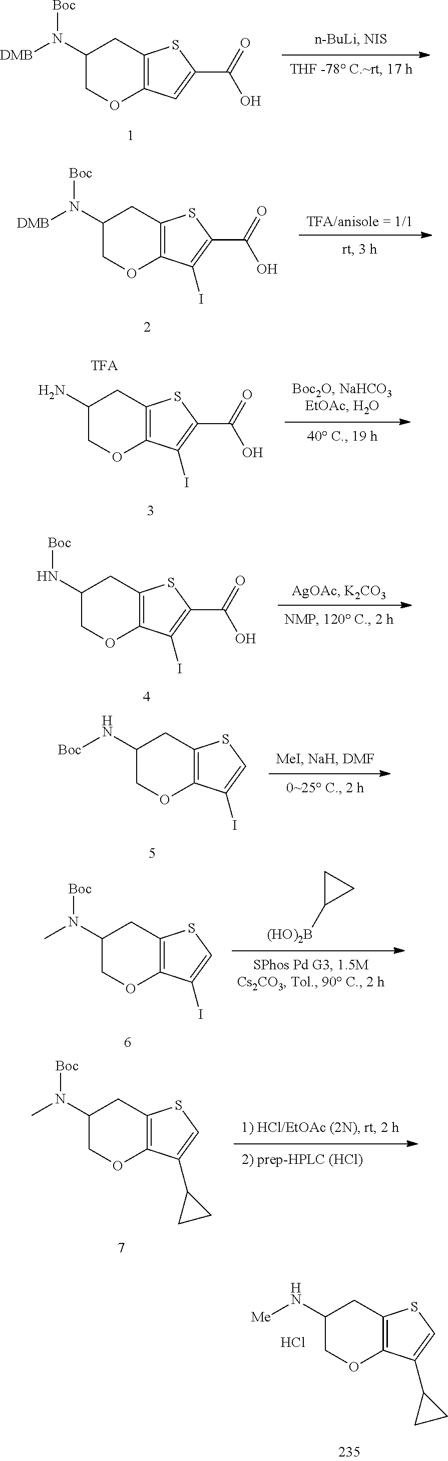

### Procedure for preparation of 6-[tert-butoxycarbonyl-[(2,4-dimethoxyphenyl)methyl]amino]-3-iodo-6,7-dihydro-5H-thieno[3,2-b]pyran-2-carboxylic acid (2)

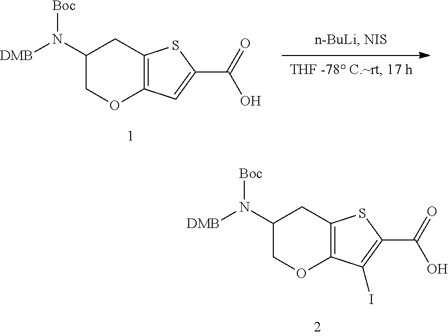

To a solution of 6-[tert-butoxycarbonyl-[(2,4-dimethoxyphenyl)methyl]amino]-6,7-dihydro-5H-thieno[3,2-b]pyran-2-carboxylic acid (1) (1.5 g, 3.34 mmol, 1 eq) in THF (10 mL) was added dropwise n-BuLi (2.5 M, 3.20 mL, 2.4 eq) at −78° C. over 0.5 hr. After addition, the mixture was stirred at this temperature for 0.5 hr, and then NIS (1.13 g, 5.01 mmol, 1.5 eq) in THF (5 mL) was added dropwise at −78° C. The mixture was allowed to warm up to rt slowly by itself and then stirred at rt for 16 hr. The mixture was then poured into aq. NH₄Cl saturated solution (50 mL) and extracted with EtOAc (20 mL*2). The combined organic phases were washed with brine (30 mL), dried over anhydrous sodium sulfate, filtered and concentrated under vacuum to give a residue. The residue was purified by prep-HPLC (column: Phenomenex luna C18 150*40 mm*15 μm; mobile phase: [water(FA)-ACN]; gradient:55%-85% B over 15 min) to afford 6-[tert-butoxycarbonyl-[(2,4-dimethoxyphenyl)methyl]amino]-3-iodo-6,7-dihydro-5H-thieno[3,2-b]pyran-2-carboxylic acid (2) (560 mg, 973.22 μmol, 29.17% yield) as a yellow solid. LC-MS: m/z [M+Na]⁺=598.0

### Procedure for preparation of 6-amino-3-iodo-6,7-dihydro-5H-thieno[3,2-b]pyran-2-carboxylic acid (3)

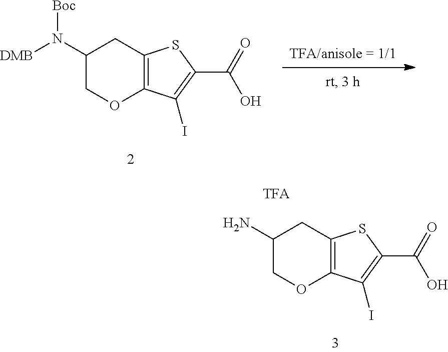

To a solution of 6-[tert-butoxycarbonyl-[(2,4-dimethoxyphenyl)methyl]amino]-3-iodo-6,7-dihydro-5H-thieno[3,2-b]pyran-2-carboxylic acid (2) (500 mg, 868.94 μmol, 1 eq) in TFA (4 mL) was added anisole (3.98 g, 36.80 mmol, 4 mL, 42.36 eq). The mixture was stirred at 25° C. for 3 hr and then poured into H₂O (5 mL) and extracted with PE (10 mL*2). The aqueous phase was collected. The organic phase was washed with H₂O (5 mL*3). The aqueous phases were combined and lyophilized to give a residue. The crude 6-amino-3-iodo-6,7-dihydro-5H-thieno[3,2-b]pyran-2-carboxylic acid TFAsalt (3) (330 mg, crude) was obtained as a yellow solid. ¹H NMR (400 MHz, METHANOL-d4) δ=4.48-4.40 (m, 1H), 4.32-4.24 (m, 1H), 3.98 (br d, J=1.8 Hz, 1H), 3.40 (br dd, J=5.8, 18.0 Hz, 1H), 2.98-2.89 (m, 1H). LC-MS: m/z [M+H]⁺=325.9

### Procedure for preparation of 6-(tert-butoxycarbonylamino)-3-iodo-6,7-dihydro-5H-thieno[3,2-b]pyran-2-carboxylic acid (4)

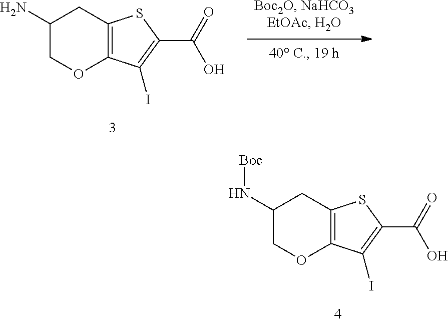

To a solution of 6-amino-3-iodo-6,7-dihydro-5H-thieno[3,2-b]pyran-2-carboxylic acid TFA salt (3) (330 mg, 1.02 mmol, 1 eq) in EtOAc (3 mL) and H₂O (1.5 mL) was added sat aq. NaHCO₃ (426.33 mg, 5.08 mmol, 197.47 μL, 5 eq) and (Boc)₂O (332.28 mg, 1.52 mmol, 349.77 μL, 1.5 eq). The mixture was stirred at 40° C. for 19 hr. Then 1 M HCl aqueous solution was added into the mixture to adjust the pH to 5 and the mixture was then extracted with EtOAc (20 mL*2). The combined organic phases were washed with brine (10 mL), dried over anhydrous sodium sulfate, filtered and concentrated under vacuum to give a residue. The residue was purified by prep-HPLC (column: Phenomenex luna C18 150*25 mm*10 μm; mobile phase: [water(TFA)-ACN]; gradient:40%-70% B over min) to afford 6-(tert-butoxycarbonylamino)-3-iodo-6,7-dihydro-5H-thieno[3,2-b]pyran-2-carboxylic acid (4) (85 mg, 199.89 μmol, 19.69% yield) as a white solid. ¹H NMR (400 MHz, CHLOROFORM-d) δ=4.91-4.82 (m, 1H), 4.30-4.18 (m, 2H), 4.13-4.04 (m, 1H), 3.06 (dd, J=5.0, 17.5 Hz, 1H), 2.77 (br dd, J=2.6, 18.8 Hz, 1H), 1.38 (s, 9H). LC-MS: m/z [M+Na]⁺=447.9

### Procedure for preparation of tert-butyl N-(3-iodo-6,7-dihydro-5H-thieno[3,2-b]pyran-6-yl)carbamate (5)

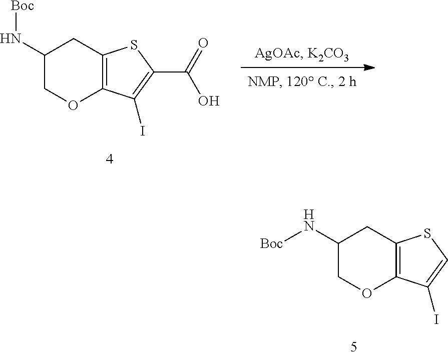

To a solution of 6-(tert-butoxycarbonylamino)-3-iodo-6,7-dihydro-5H-thieno[3,2-b]pyran-2-carboxylic acid (4) (85 mg, 199.89 μmol, 1 eq) in NMP (2 mL) was added AgOAc (6.67 mg, 39.98 μmol, 0.2 eq) and K₂CO₃ (82.88 mg, 599.66 μmol, 3 eq). The mixture was stirred at 120° C. for 2 hr. The reaction mixture was then cooled and poured into H₂O (10 mL) and extracted with EtOAc (10 mL*2). The combined organic phases were washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~20% Ethyl acetate/Petroleum ether gradient @20 mL/min) to afford tert-butyl N-(3-iodo-6,7-dihydro-5H-thieno[3,2-b]pyran-6-yl)carbamate (5) (50 mg, 131.15 μmol, 65.61% yield) as yellow oil. LC-MS: m/z [M+H-tBu]⁺=325.9

### Procedure for preparation of tert-butyl N-(3-iodo-6,7-dihydro-5H-thieno[3,2-b]pyran-6-yl)-N-methyl-carbamate (6)

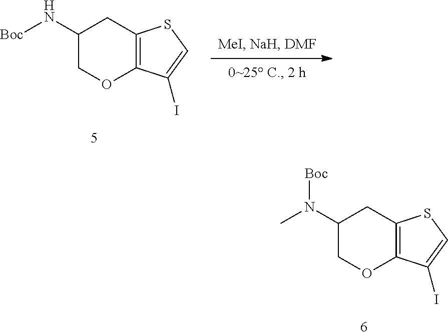

To a solution of tert-butyl N-(3-iodo-6,7-dihydro-5H-thieno[3,2-b]pyran-6-yl)carbamate (5) (50 mg, 131.15 μmol, 1 eq) in DMF (1 mL) was added dropwise NaH (10.49 mg, 262.31 μmol, 60% suspension in mineral oil, 2 eq) at 0° C. After addition, the mixture was stirred at this temperature for 0.5 hr, and then CH₃I (37.23 mg, 262.31 μmol, 16.33 μL, 2 eq) was added dropwise at 0° C. The resulting mixture was allowed to warm up to −25° C. and stirred for 1.5 hr. The reaction mixture was then poured into aq. NH₄Cl saturated solution (10 mL) and extracted with EtOAc (10 mL*2). The combined organic phases were washed with brine (10 mL), dried over anhydrous sodium sulfate, filtered and concentrated under vacuum to give a residue. The residue was purified by prep-TLC (SiO₂, Petroleum ether:Ethyl acetate=3:1) to afford tert-butyl N-(3-iodo-6,7-dihydro-5H-thieno[3,2-b]pyran-6-yl)-N-methyl-carbamate (6) (40 mg, 101.20 μmol, 77.16% yield) as yellow oil. LC-MS: m/z [M+H-tBu]⁺=339.8

### Procedure for preparation of tert-butyl N-(3-cyclopropyl-6,7-dihydro-5H-thieno[3,2-b]pyran-6-yl)-N-methyl-carbamate (7)

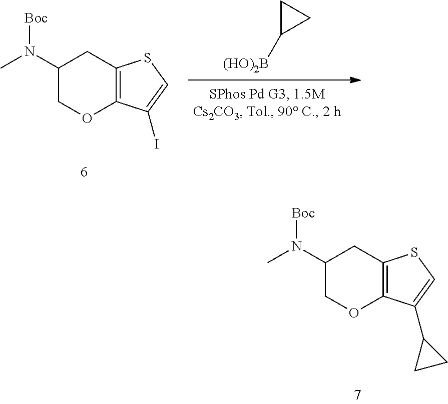 [SMILES: CN(C(=O)OC(C)(C)C)C1COc2c(I)csc2C1 ; OB(O)C1CC1 ; CN(C(=O)OC(C)(C)C)C1COc2c(C3CC3)csc2C1]

A mixture of tert-butyl N-(3-iodo-6,7-dihydro-5H-thieno[3,2-b]pyran-6-yl)-N-methyl-carbamate (6) (40 mg, 101.20 μmol, 1 eq), cyclopropylboronic acid (43.46 mg, 506.00 μmol, 5 eq), SPhos Pd G3 (7.90 mg, 10.12 μmol, 0.1 eq) and Cs₂CO₃ (1.5 M, 202.40 L, 3 eq) in Tol. (2 mL) was degassed and purged with N₂ for 3 times, and then the mixture was stirred at 90° C. for 2 hr under N₂ atmosphere. The reaction mixture was poured into H₂O (5 mL) and extracted with EtOAc (5 mL*2). The combined organic phases were washed with brine (5 mL), dried over anhydrous sodium sulfate, filtered and concentrated under vacuum to give a residue. The residue was purified by prep-TLC (SiO₂, Petroleum ether:Ethyl acetate=5:1) to afford tert-butyl N-(3-cyclopropyl-6,7-dihydro-5H-thieno[3,2-b]pyran-6-yl)-N-methyl-carbamate (7) (24 mg, 77.56 μmol, 76.64% yield) as yellow oil. LC-MS: m/z [M+H-tBu]⁺=254.0

### Procedure for preparation of 3-cyclopropyl-N-methyl-6,7-dihydro-5H-thieno[3,2-b]pyran-6-amine hydrochloride (235)

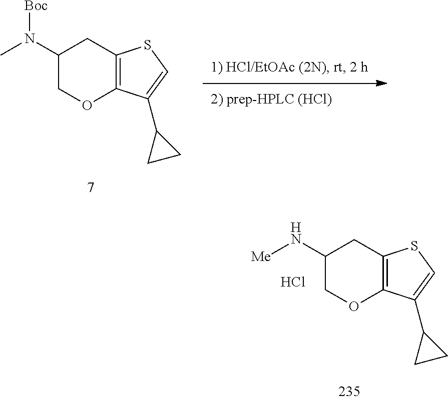

To a solution of tert-butyl N-(3-cyclopropyl-6,7-dihydro-5H-thieno[3,2-b]pyran-6-yl)-N-methyl-carbamate (7) (24 mg, 77.56 μmol, 1 eq) in EtOAc (1 mL) was added HCl/EtOAc (4 M, 1 mL, 51.57 eq). The mixture was stirred at 25° C. for 2 hr. The mixture was then concentrated and the residue was purified by prep-HPLC (column: Phenomenex luna C18 150*25 mm*10 μm; mobile phase: [water(HCl)-ACN]; gradient:1%-25% B over 10 min) to afford 3-cyclopropyl-N-methyl-6,7-dihydro-5H-thieno[3,2-b]pyran-6-amine hydrochloride (235) (16.03 mg, 63.92 μmol, 82.41% yield) as a white solid. ¹H NMR (400 MHz, METHANOL-d4) δ=6.67 (s, 1H), 4.57-4.50 (m, 1H), 4.22 (dd, J=0.6, 12.5 Hz, 1H), 3.83-3.77 (m, 1H), 3.28 (br d, J=5.6 Hz, 1H), 2.97 (br d, J=17.5 Hz, 1H), 2.82 (s, 3H), 1.83-1.75 (m, 1H), 0.88-0.79 (m, 2H), 0.70-0.62 (m, 2H). LC-MS: m/z [M+H]⁺=210.0

### Synthetic scheme for the synthesis of 3-(aminomethyl)-N-methyl-4,5,6,7-tetrahydrobenzothiophen-5-amine dihydrochloride enantiomer 1 (236) and enantiomer 2 (237)

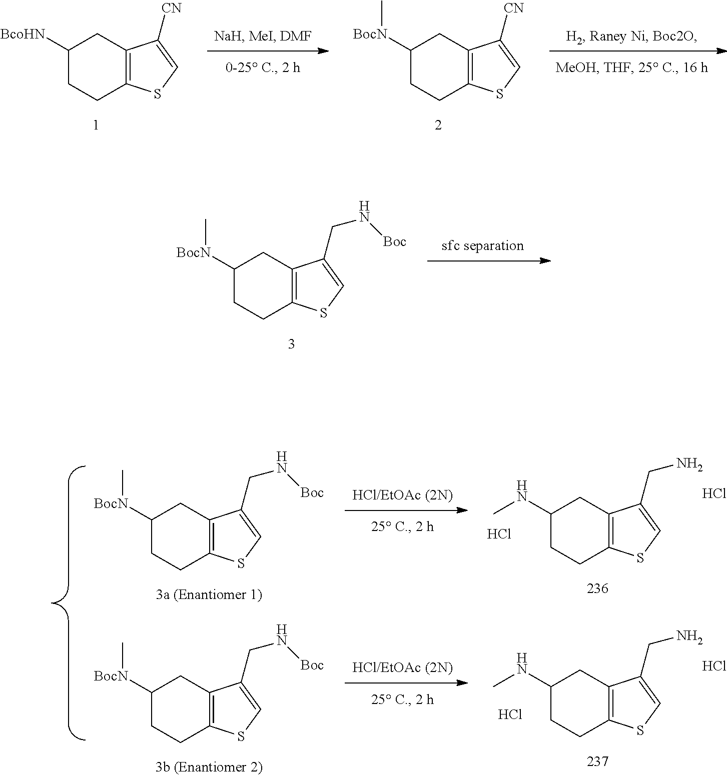

### Procedure for preparation of tert-butyl N-(3-cyano-4,5,6,7-tetrahydrobenzothiophen-5-yl)-N-methyl-carbamate (2)

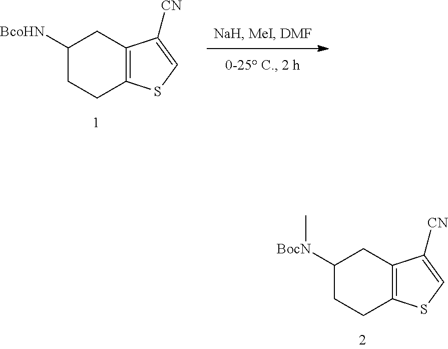

To a solution of tert-butyl N-(3-cyano-4,5,6,7-tetrahydrobenzothiophen-5-yl)carbamate (1) (500 mg, 1.80 mmol, 1 eq) in DMF (5 mL) was added NaH (143.68 mg, 3.59 mmol, 60% suspension in mineral oil, 2 eq) at 0° C. The mixture was stirred at this temperature for 0.5 hr, and then CH₃I (382.42 mg, 2.69 mmol, 167.73 μL, 1.5 eq) was added dropwise at 0° C. The resulting mixture was warmed to 25° C. and stirred for 1.5 hr. The reaction mixture was then poured into aq. NH₄Cl saturated solution (10 mL) and extracted with EtOAc (10 mL*2). The combined organic phases were washed with brine (10 mL), dried over anhydrous sodium sulfate, filtered and concentrated under vacuum to give a residue. The crude product was suspended in PE(5 mL) at 25° C. for 30 min. The mixture was then filtered and, the filter cake was dried to give tert-butyl N-(3-cyano-4,5,6,7-tetrahydrobenzothiophen-5-yl)-N-methyl-carbamate (2) (460 mg, 1.57 mmol, 87.59% yield) as a yellow solid. ¹H NMR (400 MHz, CHLOROFORM-d) δ=7.73 (s, 1H), 4.61-4.21 (m, 1H), 3.03-2.65 (m, 7H), 2.07-1.86 (m, 2H), 1.49 (s, 9H). LC-MS: m/z [M+H-tBu]⁺=237.0

### Procedure for preparation of tert-butyl N-[3-[(tert-butoxycarbonylamino)methyl]-4,5,6,7-tetrahydrobenzothiophen-5-yl]-N-methyl-carbamate (3)

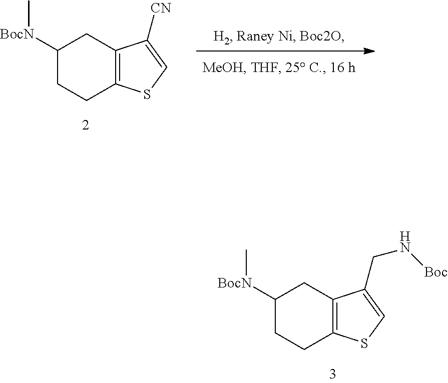 [SMILES: CN(C(=O)OC(C)(C)C)C1CCc2scc(C#N)c2C1 ; CN(C(=O)OC(C)(C)C)C1CCc2scc(CNC(=O)OC(C)(C)C)c2C1]

To a solution of tert-butyl N-(3-cyano-4,5,6,7-tetrahydrobenzothiophen-5-yl)-N-methyl-carbamate (2) (200 mg, 684.00 μmol, 1 eq) in MeOH (5 mL) was added (Boc)₂O (298.56 mg, 1.37 mmol, 314.28 μL, 2 eq) and Raney-Ni (70.32 mg, 820.81 μmol, 1.2 eq) under N₂ atmosphere. The suspension was degassed and purged with H₂ for 3 times. The mixture was stirred under H₂ (15 Psi) at 25° C. for 16 hr. The mixture was followed by filtration, the filtrate was concentrated. The residue was purified by prep-HPLC (column: Waters Xbridge Prep OBD C18 150*40 mm*10 μm; mobile phase: [water(NH₄HCO₃)-ACN]; gradient:50%-80% B over 15 min) to give tert-butyl N-[3-[(tert-butoxycarbonylamino)methyl]-4,5,6,7-tetrahydrobenzothiophen-5-yl]-N-methyl-carbamate (3) (170 mg, 428.71 μmol, 62.68% yield) as a red solid. ¹H NMR (400 MHz, CHLOROFORM-d) δ=6.92 (s, 1H), 4.73-4.55 (m, 1H), 4.50-4.28 (m, 1H), 4.25-4.10 (m, 2H), 2.91 (br d, J=5.6 Hz, 2H), 2.80 (s, 3H), 2.70-2.62 (m, 1H), 2.60-2.50 (m, 1H), 2.00-1.85 (m, 2H), 1.47 (d, J=7.8 Hz, 18H)

### Procedure for preparation of tert-butyl N-[3-[(tert-butoxycarbonylamino)methyl]-4,5,6,7-tetrahydrobenzothiophen-5-yl]-N-methyl-carbamate enantiomer 1 (3a) and enantiomer 2 (3b)

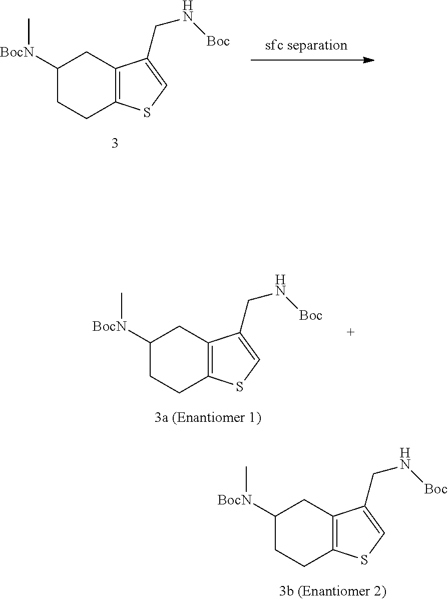 [SMILES: CN(C(=O)OC(C)(C)C)C1CCc2scc(CNC(=O)OC(C)(C)C)c2C1 ; CN(C(=O)OC(C)(C)C)C1CCc2scc(CNC(=O)OC(C)(C)C)c2C1 ; CN(C(=O)OC(C)(C)C)C1CCc2scc(CNC(=O)OC(C)(C)C)c2C1]

Racemic tert-butyl N-[3-[(tert-butoxycarbonylamino)methyl]-4,5,6,7-tetrahydrobenzothiophen-5-yl]-N-methyl-carbamate (3) was separated to enantiomers by SFC (column: DAICEL CHIRALPAK IG (250 mm*50 mm, 10 μm); mobile phase: [CO₂-MeOH (0.1% NH₃H₂O)]; B %:20%, isocratic elution mode) to afford tert-butyl N-[3-[(tert-butoxycarbonylamino)methyl]-4,5,6,7-tetrahydrobenzothiophen-5-yl]-N-methyl-carbamate enantiomer 1 (3a) (85 mg, 214.35 μmol, 50.00% yield) as yellow oil and tert-butyl N-[3-[(tert-butoxycarbonylamino)methyl]-4,5,6,7-tetrahydrobenzothiophen-5-yl]-N-methyl-carbamate enantiomer 2 (3b) (85 mg, 214.35 μmol, 50.00% yield) as yellow oil.

### Procedure for preparation of 3-(aminomethyl)-N-methyl-4,5,6,7-tetrahydrobenzothiophen-5-amine dihydrochloride enantiomer 1 (236)

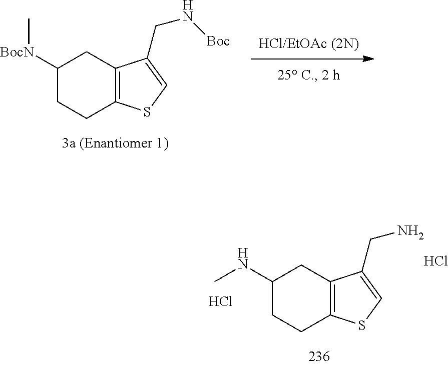 [SMILES: CN(C(=O)OC(C)(C)C)C1CCc2scc(CNC(=O)OC(C)(C)C)c2C1 ; Cl ; CNC1CCc2scc(CN)c2C1 ; Cl]

To a solution of tert-butyl N-[3-[(tert-butoxycarbonylamino)methyl]-4,5,6,7-tetrahydrobenzothiophen-5-yl]-N-methyl-carbamate enantiomer 1 (3a) (85 mg, 214.35 μmol, 1 eq) in EtOAc (1 mL) was added HCl/EtOAc (4 M, 1 mL, 18.66 eq). The mixture was stirred at 25° C. for 2 hr and then it was concentrated. The residue was triturated with EtOAc (1 mL) at 25° C. for 10 min then collected by filtration and dried to afford 3-(aminomethyl)-N-methyl-4,5,6,7-tetrahydrobenzothiophen-5-amine dihydrochloride enantiomer 1 (236) (30.52 mg, 112.23 μmol, 52.36% yield) as an off-white solid. ¹H NMR (400 MHz, METHANOL-d4) δ=7.41 (s, 1H), 4.04-3.97 (m, 4H), 2.96 (t, J=6.6 Hz, 2H), 2.82 (s, 2H), 1.98 (t, J=6.6 Hz, 2H). LC-MS: m/z [M+H]⁺=197.2

### Procedure for preparation of 3-(aminomethyl)-N-methyl-4,5,6,7-tetrahydrobenzothiophen-5-amine dihydrochloride enantiomer 2 (237)

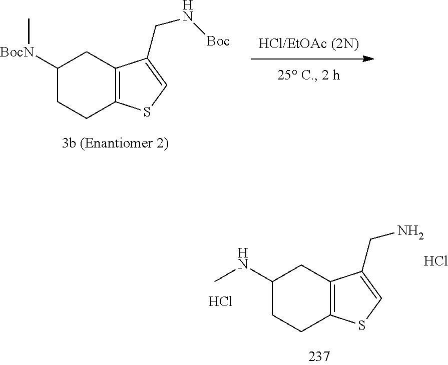 [SMILES: CN(C(=O)OC(C)(C)C)C1CCc2scc(CNC(=O)OC(C)(C)C)c2C1 ; Cl ; CNC1CCc2scc(CN)c2C1 ; Cl]

To a solution of tert-butyl N-[3-[(tert-butoxycarbonylamino)methyl]-4,5,6,7-tetrahydrobenzothiophen-5-yl]-N-methyl-carbamate enantiomer 2 (3b) (85 mg, 214.35 μmol, 1 eq) in EtOAc (1 mL) was added HCl/EtOAc (4 M, 1 mL, 18.66 eq). The mixture was stirred at 25° C. for 2 hr and then concentrated. The residue was triturated with EtOAc (1 mL) at 25° C. for 10 min then collected by filtration and dried to afford 3-(aminomethyl)-N-methyl-4,5,6,7-tetrahydrobenzothiophen-5-amine dihydrochloride enantiomer 2 (237) (38.43 mg, 141.31 μmol, 65.92% yield) as an orange solid. ¹H NMR (400 MHz, DMSO-d6) δ=9.49-9.20 (m, 2H), 8.65-8.28 (m, 3H), 7.44 (s, 1H), 3.97-3.80 (m, 2H), 3.42-3.35 (m, 1H), 3.13 (br dd, J=5.1, 15.5 Hz, 1H), 2.98-2.87 (m, 1H), 2.85-2.73 (m, 1H), 2.72-2.65 (m, 1H), 2.63 (s, 3H), 2.34-2.23 (m, 1H), 1.88 (dq, J=5.7, 11.5 Hz, 1H). LC-MS: m/z [M+H]⁺=197.2

### Synthetic scheme for preparation of N, 3,6-trimethyl-6,7-dihydro-5H-thieno[3,2-b]pyran-6-amine hydrochloride racemate(238), enantiomer 1 (239) and enantiomer 2 (240)

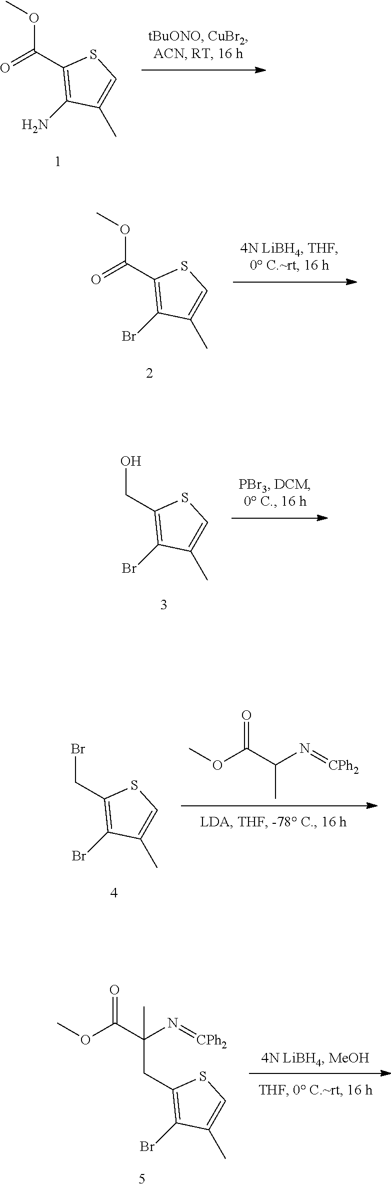

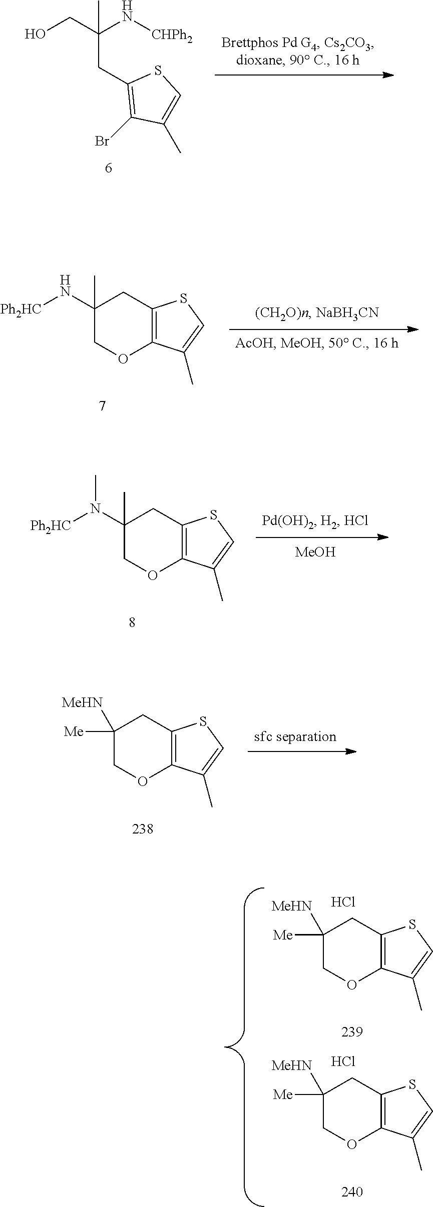

### Procedure for preparation of methyl 3-bromo-4-methylthiophene-2-carboxylate (2)

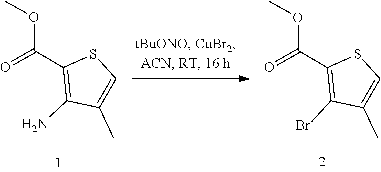

To a stirred suspension of CuBr₂ (15 g, 67.16 mmol, 1.15 eq) in ACN (70 mL) was added tert-butyl nitrite (7.80 g, 75.67 mmol, 9 mL, 1.30 eq) under N₂, then methyl 3-amino-4-methyl-thiophene-2-carboxylate (1) (10 g, 58.41 mmol, 1 eq) in ACN (30 mL) was added. The reaction mixture was stirred at 25° C. for 16 hr. The reaction mixture was then filtered and concentrated under reduced pressure to give a residue. The residue was purified by flash silica gel chromatography (ISCO®; 80 g SepaFlash® Silica Flash Column, eluent 0~6% Ethyl acetate/Petroleum ether gradient @100 mL/min). Compound methyl 3-bromo-4-methyl-thiophene-2-carboxylate (2) (9 g, 38.28 mmol, 65.54% yield) was obtained as a white solid. ¹H NMR (400 MHz, METHANOL-d4) δ=7.48 (s, 1H), 3.86 (s, 3H), 2.25 (s, 3H).

### Procedure for preparation of (3-bromo-4-methylthiophen-2-yl)methanol (3)

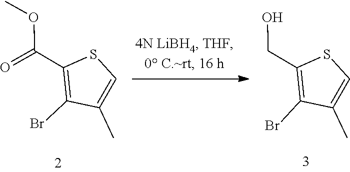

To a solution of methyl 3-bromo-4-methyl-thiophene-2-carboxylate (2) (2 g, 8.51 mmol, 1 eq) in THF (15 mL) was added LiBH₄ (4 M, 6 mL, 2.82 eq) at 0° C., then MeOH (0.5 mL) was added. Then the mixture was warmed to 25° C. and stirred for 16 hr under N₂. The reaction mixture was quenched by addition aq. sat NH₄Cl (60 mL) at 0° C., and then extracted with EtOAc (30 mL*2). The combined organic layers were washed with aq. saturated NaCl (30 mL*2), dried over anhydrous Na₂SO₄, filtered and concentrated under reduced pressure to give a residue. The residue was purified by flash silica gel chromatography (ISCO®; 12 g SepaFlash® Silica Flash Column, eluent 0~12% Ethyl acetate/Petroleum ether gradient @40 mL/min). Compound (3-bromo-4-methyl-2-thienyl)methanol (3) (1.6 g, 7.73 mmol, 90.82% yield) was obtained as a white solid. ¹H NMR (400 MHz, DMSO-d6) δ=7.27 (d, J=0.6 Hz, 1H), 5.61 (t, J=5.6 Hz, 1H), 4.56 (d, J=5.6 Hz, 2H), 2.14 (s, 3H).

### Procedure for preparation of 3-bromo-2-(bromomethyl)-4-methylthiophene (4)

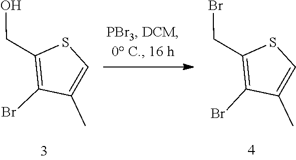

To a solution of (3-bromo-4-methyl-2-thienyl) methanol (3) (1.6 g, 7.73 mmol, 1 eq) in DCM (15 mL) was added PBr₃ (2.5 g, 9.24 mmol, 1.20 eq) at 0° C. The mixture was warmed up gradually to 25° C. and stirred for 16 hr. Then the mixture was quenched by addition of saturated NaHCO₃ (aq, 50 mL), and extracted with DCM (20 mL*3). The combined organic layers were washed with brine (30 mL*2), dried over anhydrous Na₂SO₄, filtered and concentrated under reduced pressure to give a residue. The residue was purified by flash silica gel chromatography (ISCO®; 20 g SepaFlash® Silica Flash Column, eluent 0~5% Ethyl acetate/Petroleum ether gradient @40 mL/min).Compound 3-bromo-2-(bromomethyl)-4-methyl-thiophene (4) (1.4 g, 5.19 mmol, 67.12% yield) was obtained as a light yellow solid. ¹H NMR (400 MHz, DMSO-d6) δ=7.47 (s, 1H), 4.88 (s, 2H), 2.16 (d, J=0.7 Hz, 3H)

### Procedure for preparation of methyl 3-(3-bromo-4-methylthiophen-2-yl)-2-((diphenylmethylene)amino)-2-methylpropanoate(5)

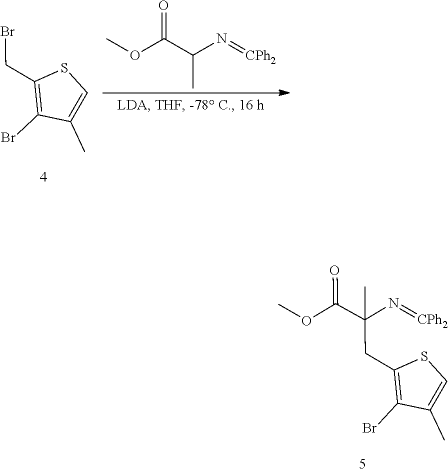

To a solution of LDA (2 M, 2.50 mL, 1.50 eq) in THF (15 mL) was added methyl 2-(benzhydrylideneamino)propanoate (4) (1.34 g, 5.00 mmol, 1.5 eq) at −78° C. dropwise, the mixture was stirred at this temperature for 1 hr, and then 3-bromo-2-(bromomethyl)-4-methyl-thiophene (900 mg, 3.33 mmol, 1 eq) was added. Then the mixture was allowed to naturally and gradually warm up to 20° C. and stirred at that temperature for 15 hr under N₂. The reaction mixture was quenched by addition of sat. NH₄Cl (aq, 60 mL) at 0° C., and then extracted with EtOAc (30 mL*2). The combined organic layers were washed with aq. saturated NaCl (30 mL*2), dried over anhydrous Na₂SO₄, filtered and concentrated under reduced pressure to give a residue. The residue was purified by flash silica gel chromatography (ISCO®; 20 g SepaFlash® Silica Flash Column, eluent 0~3% Ethyl acetate/Petroleum ether gradient @40 mL/min). Compound methyl 2-(benzhydrylideneamino)-3-(3-bromo-4-methyl-2-thienyl)-2-methyl-propanoate (5) (1.4 g, 2.56 mmol, 76.65% yield) was obtained as a light yellow oil. ¹H NMR (400 MHz, METHANOL-d4) δ=7.61 (d, J=7.6 Hz, 2H), 7.45-7.37 (m, 4H), 7.36-7.29 (m, 2H), 7.16-7.09 (m, 2H), 7.07 (s, 1H), 3.55-3.49 (m, 1H), 3.45-3.40 (m, 1H), 3.35 (s, 3H), 2.20 (s, 3H), 1.32 (s, 3H). LC-MS: m/z [M+H]⁺=458.0

### Procedure for preparation of 2-(benzhydrylamino)-3-(3-bromo-4-methylthiophen-2-yl)-2-methylpropan-1-ol (6)

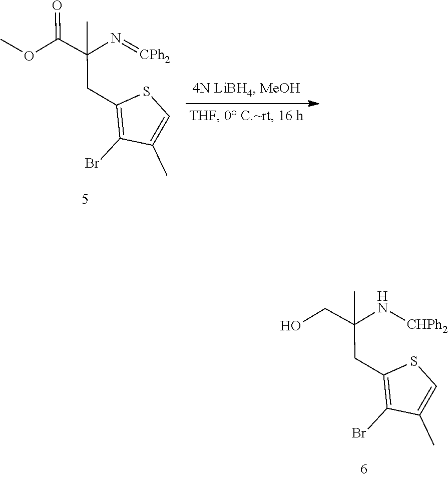

To a solution of methyl 2-(benzhydrylideneamino)-3-(3-bromo-4-methyl-2-thienyl)-2-methyl-propanoate (5) (1.4 g, 3.07 mmol, 1 eq) in THF (15 mL) was added LiBH₄ (4 M, 4 mL, 5.22 eq) at 0° C. and then MeOH (0.5 mL) under N₂. Then the mixture was allowed to naturally rise to 25° C. and stirred for 16 hr under N₂. The reaction mixture was then quenched by addition of aq. sat. NH₄Cl 50 mL at 0° C., and then extracted with EtOAc (20 mL*3). The combined organic layers were washed with saturated aq. NaCl (30 mL*2), dried over anhydrous Na₂SO₄, filtered and concentrated under reduced pressure to give a residue. The residue was purified by flash silica gel chromatography (ISCO®; 20 g SepaFlash® Silica Flash Column, eluent 0~12% Ethyl acetate/Petroleum ether gradient @60 mL/min). Compound 2-(benzhydrylamino)-3-(3-bromo-4-methyl-2-thienyl)-2-methyl-propan-1-ol (6) (1.2 g, 2.70 mmol, 87.89% yield) was obtained as a light yellow oil. ¹H NMR (400 MHz, METHANOL-d4) δ=7.44 (d, J=8.1 Hz, 4H), 7.30-7.18 (m, 4H), 7.18-7.09 (m, 2H), 7.04 (d, J=1.1 Hz, 1H), 5.08 (s, 1H), 3.52-3.37 (m, 2H), 3.06-3.00 (m, 1H), 2.96-2.91 (m, 1H), 2.19 (d, J=1.0 Hz, 3H), 0.86 (s, 3H). LC-MS: m/z [M+H]⁺=432.2

### Preparation of N-benzhydryl-3,6-dimethyl-6,7-dihydro-5H-thieno[3,2-b]pyran-6-amine (7)

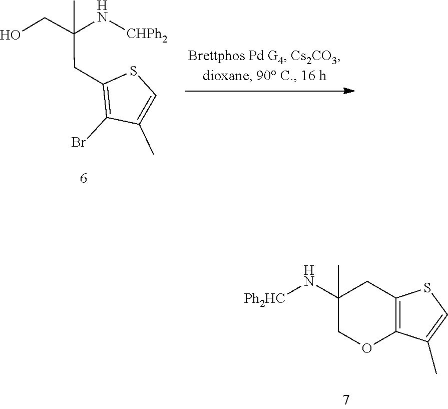

A mixture of 2-(benzhydrylamino)-3-(3-bromo-4-methyl-2-thienyl)-2-methyl-propan-1-ol (6) (1.2 g, 2.79 mmol, 1 eq), BrettPhos Pd, G4 (256.65 mg, 278.81 μmol, 0.1 eq), Cs₂CO₃ (2.27 g, 6.97 mmol, 2.5 eq) in dioxane (50 mL) was degassed and purged with N₂ for 3 times, and stirred at 90° C. for 4 hr under N₂ atmosphere. The reaction mixture was then concentrated under reduced pressure to give a residue. The residue was purified by flash silica gel chromatography (ISCO®; 12 g SepaFlash® Silica Flash Column, eluent 0-12% Ethyl acetate/Petroleum ether gradient @40 mL/min). Compound N-benzhydryl-3,6-dimethyl-5,7-dihydrothieno[3,2-b]pyran-6-amine (7) (860 mg, 2.34 mmol, 83.85% yield) was obtained as a light yellow oil. ¹H NMR (400 MHz, METHANOL-d4) δ=7.38 (d, J=7.5 Hz, 2H), 7.32-7.12 (m, 8H), 6.67 (s, 1H), 5.12 (s, 1H), 3.99 (dd, J=1.5, 10.8 Hz, 1H), 3.73 (d, J=10.6 Hz, 1H), 2.82 (d, J=16.0 Hz, 1H), 2.57 (d, J=15.9 Hz, 1H), 1.99 (s, 3H), 1.04 (s, 3H). LC-MS: m/z [M+H]⁺=350.3

### Preparation of N-benzhydryl-N, 3,6-trimethyl-6,7-dihydro-5H-thieno[3,2-b]pyran-6-amine (8)

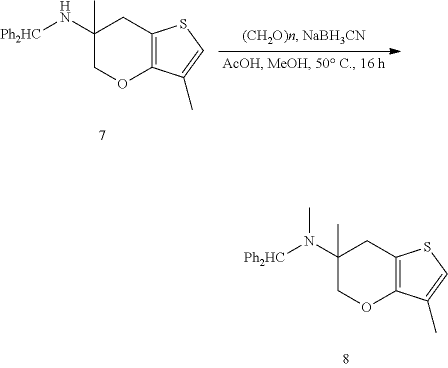

To a mixture of N-benzhydryl-3,6-dimethyl-5,7-dihydrothieno[3,2-b]pyran-6-amine (7) (860 mg, 2.46 mmol, 1 eq), aq. HCHO (400 mg, 13.32 mmol, 366.97 μL, 5.41 eq) and AcOH (1.05 g, 17.47 mmol, 1 mL, 7.10 eq) in MeOH (10 mL), was added NaBH₃CN (300 mg, 4.77 mmol, 1.94 eq). The mixture was purged with N₂, and then stirred at 50° C. for 16 hr under N₂ atmosphere. The reaction mixture was quenched by addition of aq. sat. NaHCO₃ (20 mL) at 0° C., then concentrated under reduced pressure to remove MeOH, and then extracted with EtOAc (15 mL*3). The combined organic layers were washed with saturated aq. NaCl (30 mL*2), dried over anhydrous Na₂SO₄, filtered and concentrated under reduced pressure to give a residue that was purified by reverse-phase HPLC (0.1% FA condition). Compound N-benzhydryl-N, 3,6-trimethyl-5,7-dihydrothieno[3,2-b]pyran-6-amine (8) (400 mg, 1.05 mmol, 42.84% yield) was obtained as a white solid. ¹H NMR (400 MHz, METHANOL-d4) δ=7.26-7.14 (m, 10H), 6.67 (d, J=0.8 Hz, 1H), 5.51 (s, 1H), 4.25 (dd, J=1.9, 11.1 Hz, 1H), 3.93 (d, J=11.1 Hz, 1H), 3.07 (dd, J=1.4, 15.9 Hz, 1H), 2.62 (d, J=16.0 Hz, 1H), 2.30 (s, 3H), 2.01 (d, J=1.0 Hz, 3H), 1.00 (s, 3H). LC-MS: m/z [M+H]⁺=364.2.

### N, 3,6-trimethyl-6,7-dihydro-5H-thieno[3,2-b]pyran-6-amine hydrochloride racemate (238)

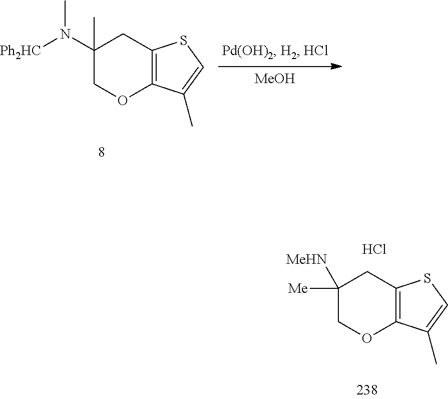

To a solution of N-benzhydryl-N, 3,6-trimethyl-5,7-dihydrothieno[3,2-b]pyran-6-amine (8) (350 mg, 962.82 μmol, 1 eq) in MeOH (15 mL) was added Pd(OH)₂ (70.00 mg, 99.69 μmol, 20% purity, 1.04e-1 eq) and aq. HCl (12 M, 0.5 mL, 6.23 eq) under Ar. The suspension was degassed under vacuum and purged with H₂ several times. The mixture was stirred under H₂ 50 Psi at 25° C. for 16 hours. The reaction mixture was then filtered and filtrate was concentrated under reduced pressure to give a residue. The residue was purified by prep-HPLC (column: Phenomenex luna C18 150*25 mm*10 μm; mobile phase: [water(HCl)-ACN]; gradient:1%-25% B over 10 min). Compound N, 3,6-trimethyl-5,7-dihydrothieno[3,2-b]pyran-6-amine hydrochloride racemate (238) (60.92 mg, 257.59 μmol, 26.75% yield) was obtained as a brown solid. ¹H NMR (400 MHz, METHANOL-d4) δ=6.88 (s, 1H), 4.36 (dd, J=2.0, 12.3 Hz, 1H), 4.03 (s, 1H), 3.11-2.99 (m, 2H), 2.71 (s, 3H), 2.08 (d, J=1.0 Hz, 3H), 1.45 (s, 3H). LC-MS: m/z [M+H]⁺=198.1

### N, 3,6-trimethyl-6,7-dihydro-5H-thieno[3,2-b]pyran-6-amine hydroachloride enantiomer 1 (239) and enantiomer 2 (240)

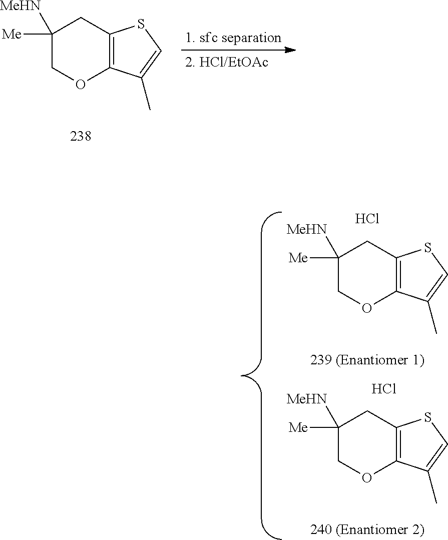

Racemic N, 3,6-trimethyl-5,7-dihydrothieno[3,2-b]pyran-6-amine free base (238) separated to its enantiomers by SFC (column: DAICEL CHIRALPAK IG (250 mm*50 mm, 10 μm); mobile phase: [CO₂-i-PrOH HEXANE (0.1% NH3H2O)]; B %:17%, isocratic elution mode). Enantiomer1, 1^{st} peak in SFC (Rt=1.115 min) and enantiomer 2, 2nd peak in SFC, (Rt=1.182 min). To each of the enantiomers isolated was added HCl/EtOAc (1 mL) and after this addition each mixture formed concentrated to give the HCl salt of each enantiomer Compound N, 3,6-trimethyl-5,7-dihydrothieno[3,2-b]pyran-6-amine hydrochloride enantiomer 1 (239) (13.61 mg, 57.48 μmol, 28.35% yield) was obtained as a brown solid. ¹H NMR (400 MHz, METHANOL-d4) δ=6.88 (s, 1H), 4.36 (dd, J=2.0, 12.3 Hz, 1H), 4.02 (d, J=12.4 Hz, 1H), 3.11-2.99 (m, 2H), 2.71 (s, 3H), 2.08 (d, J=0.9 Hz, 3H), 1.45 (s, 3H). LC-MS: m/z [M+H]⁺=198.1. Compound N, 3,6-trimethyl-5,7-dihydrothieno[3,2-b]pyran-6-amine hydrochloride enantiomer 2 (240) (10.86 mg, 45.75 μmol, 22.57% yield) was obtained as off-white solid. ¹H NMR (400 MHz, METHANOL-d4) δ=6.87 (s, 1H), 4.36 (dd, J=2.0, 12.3 Hz, 1H), 4.02 (d, J=12.3 Hz, 1H), 3.11-2.99 (m, 2H), 2.71 (s, 3H), 2.08 (d, J=0.9 Hz, 3H), 1.45 (s, 3H). LC-MS: m/z [M+H]⁺=198.1.

### Synthetic scheme for preparation of 6-methyl-3-(trifluoromethyl)-5,7-dihydro-4H-benzothiophen-6-amine hydrochloride enantiomer 1 (241) and enantiomer 2 (242)

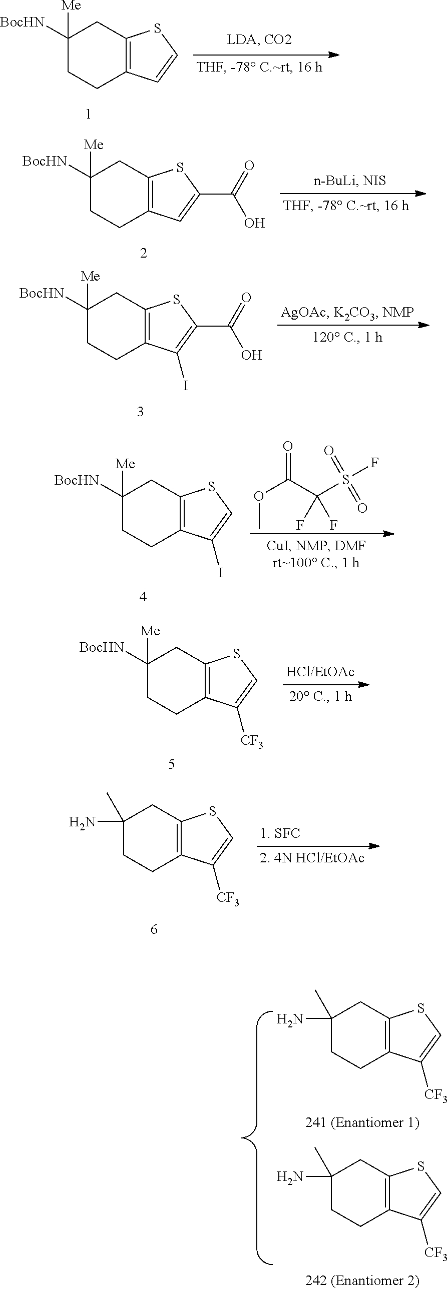

### Procedure for preparation of 6-(tert-butoxycarbonylamino)-6-methyl-5,7-dihydro-4H-benzothiophene-2-carboxylic acid (2)

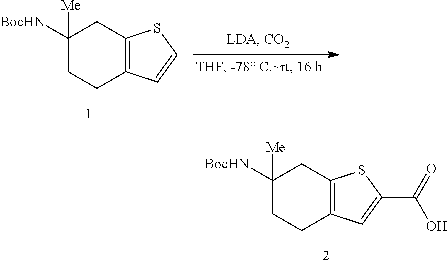 [SMILES: CC1(NC(=O)OC(C)(C)C)CCc2ccsc2C1 ; CC1(NC(=O)OC(C)(C)C)CCc2cc(C(=O)O)sc2C1]

To a mixture of tert-butyl N-(6-methyl-5,7-dihydro-4H-benzothiophen-6-yl)carbamate (1) (7.2 g, 26.93 mmol, 1 eq) in THF (90 mL) was added LDA (2 M, 30.97 mL, 2.3 eq) at −78° C. under N₂. The mixture was stirred at 78° C. for 0.5 h. Then CO₂ was bubbled into the mixture and the reaction was purged with CO₂ several times. The mixture was stirred under CO₂ atmosphere at−RT for 15.5 h. The reaction mixture was then quenched by addition H₂O 200 mL at 0° C., and then extracted with EtOAc 150 mL. The combined organic layers were washed with aq. sat. NaCl (40 mL), dried over anhydrous Na₂SO₄, filtered and concentrated under reduced pressure to recover any unreacted tert-butyl N-(6-methyl-5,7-dihydro-4H-benzothiophen-6-yl)carbamate (1). The aqueous phase pH was adjusted to 5-6 by 1N aq HCl and then extracted with EtOAc 150 mL (50*3 mL). The combined organic layers were washed with aq. sat. NaCl (40 mL), dried over anhydrous Na₂SO₄, filtered and concentrated under reduced pressure to give a residue that was purified by flash silica gel chromatography (ISCO®; 20 g SepaFlash® Silica Flash Column, eluent 0~8% Ethyl acetate/Petroleum ether gradient @50 mL/min). Compound 6-(tert-butoxycarbonylamino)-6-methyl-5,7-dihydro-4H-benzothiophene-2-carboxylic acid (2) (1.95 g, 6.26 mmol, 23.26% yield) was obtained as a white solid.

### Procedure for preparation of 6-(tert-butoxycarbonylamino)-3-iodo-6-methyl-5,7-dihydro-4H-benzothiophene-2-carboxylic acid (3)

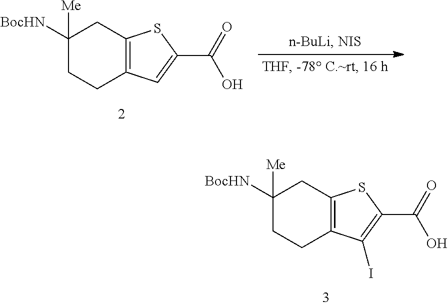 [SMILES: CC1(NC(=O)OC(C)(C)C)CCc2cc(C(=O)O)sc2C1 ; CC1(NC(=O)OC(C)(C)C)CCc2c(sc(C(=O)O)c2I)C1]

To a solution of 6-(tert-butoxycarbonylamino)-6-methyl-5,7-dihydro-4H-benzothiophene-2-carboxylic acid (2) (4.45 g, 14.29 mmol, 1 eq) in THF (50 mL) was added n-BuLi (2.5 M, 18.86 mL, 3.3 eq). Mixture was stirred at −78° C. for 1 h and then NIS (4.18 g, 18.58 mmol, 1.3 eq) dissolved in THF (5 mL) was added in the mixture at −78° C. The mixture was allowed to warm up naturally to rt and stirred for 16 hr. The reaction mixture was then quenched by addition aq. sat. NH₄Cl (400 mL) at 0° C., diluted with H₂O and extracted with EtOAc (50 mL*2). The combined organic layers were washed with aq. sat. NaCl (40 mL), dried over anhydrous Na₂SO₄, filtered and concentrated under reduced pressure. Compound 6-(tert-butoxycarbonylamino)-3-iodo-6-methyl-5,7-dihydro-4H-benzothiophene-2-carboxylic acid (3) (6.2 g, 14.18 mmol, 99.21% yield) was obtained as a yellow solid.

### Procedure for preparation of tert-butyl N-(3-iodo-6-methyl-5,7-dihydro-4H-benzothiophen-6-yl)carbamate (12)

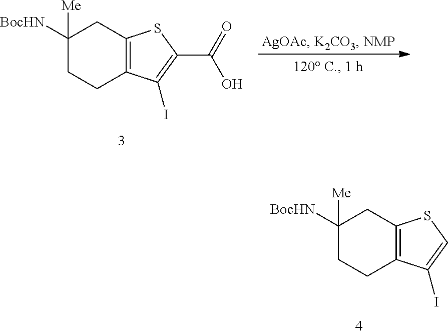 [SMILES: CC1(NC(=O)OC(C)(C)C)CCc2c(sc(C(=O)O)c2I)C1 ; CC1(NC(=O)OC(C)(C)C)CCc2c(I)csc2C1]

To a solution of 6-(tert-butoxycarbonylamino)-3-iodo-6-methyl-5,7-dihydro-4H-benzothiophene-2-carboxylic acid (3) (6.2 g, 14.18 mmol, 1 eq) in NMP (65 mL) was added AgOAc (591.62 mg, 3.54 mmol, 0.25 eq) and K₂CO₃ (5.88 g, 42.53 mmol, 3 eq). The mixture was stirred at 120° C. for 1 hr. The reaction mixture was concentrated under reduced pressure diluted with EtOAc (20 mL) and filtered through a pad of diatomite. The filtrate was diluted with H₂O 30 mL and extracted with EtOAc (10 mL*3). The combined organic layers were washed with brine (10 mL*3), dried over anhydrous sodium sulfate, filtered and concentrated under reduced pressure and the residue was purified by flash silica gel chromatography (ISCO®; 20 g SepaFlash® Silica Flash Column, eluent 0~10% Ethyl acetate/Petroleum ether gradient @40 mL/min). Compound tert-butyl N-(3-iodo-6-methyl-5,7-dihydro-4H-benzothiophen-6-yl)carbamate (4) (0.64 g, 1.26 mmol, 8.88% yield) was obtained as a yellow oil. LC-MS: m/z [M+H]⁺=338.0.

### Procedure for preparation of tert-butyl N-[6-methyl-3-(trifluoromethyl)-5,7-dihydro-4H-benzothiophen-6-yl]carbamate (5)

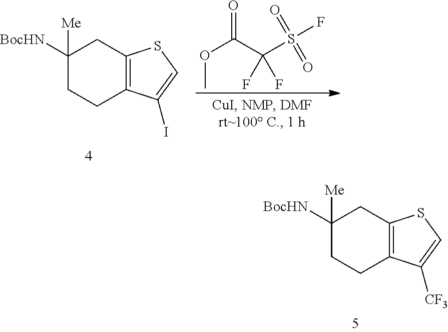

To a solution of tert-butyl N-(3-iodo-6-methyl-5,7-dihydro-4H-benzothiophen-6-yl)carbamate (4) (0.64 g, 1.63 mmol, 1 eq) in DMF (8 mL) was added methyl 2,2-difluoro-2-fluorosulfonyl-acetate (1.56 g, 8.14 mmol, 1.04 mL, 5 eq) CuI (1.55 g, 8.14 mmol, 5 eq) and NMP (241.98 mg, 2.44 mmol, 237.23 μL, 1.5 eq). The mixture was stirred at 100° C. for 1 hr. The reaction mixture was then cooled, concentrated under reduced pressure diluted with EtOAc (20 mL) and filtered through a pad of diatomite. The filtrate was diluted with H₂O (30 mL) and extracted with EtOAc (10 mL*3). The combined organic layers were washed with brine (10 mL*3), dried over anhydrous sodium sulfate, filtered and concentrated under reduced pressure. The residue was purified by flash silica gel chromatography (ISCO®; 20 g SepaFlash® Silica Flash Column, eluent 0~10% Ethyl acetate/Petroleum ether gradient @50 mL/min) and by prep-HPLC (column: Waters Xbridge 150*25 mm*5 μm; mobile phase: [water(NH₄HCO₃)-ACN]; gradient:60%-90% B over 9 min). Compound tert-butyl N-[6-methyl-3-(trifluoromethyl)-5,7-dihydro-4H-benzothiophen-6-yl]carbamate (5) (330 mg, 983.95 μmol, 60.46% yield) was obtained as a white solid. LC-MS: m/z [M+H-tBu]⁺=280.0.

### Procedure for preparation of 6-methyl-3-(trifluoromethyl)-5,7-dihydro-4H-benzothiophen-6-amine (6)

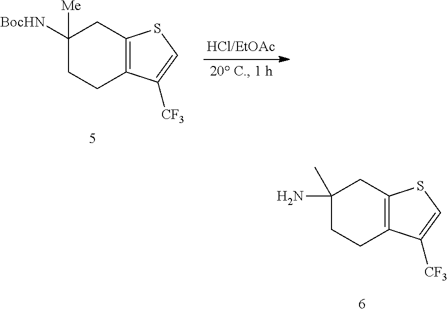 [SMILES: CC1(NC(=O)OC(C)(C)C)CCc2c(C(F)(F)F)csc2C1 ; CC1(N)CCc2c(C(F)(F)F)csc2C1]

To a solution of tert-butyl N-[6-methyl-3-(trifluoromethyl)-5,7-dihydro-4H-benzothiophen-6-yl]carbamate (5) (250 mg, 745.41 μmol, 1 eq) in EtOAc (2 mL) was added HCl/EtOAc (4 M, 4 mL, 21.46 eq). The mixture was stirred at 20° C. for 1 hr. The reaction mixture was concentrated under vacuum. The residue was diluted with 10 mL MeOH and anion exchange resin (AMBERLITE(R)IRA-400 (CL, cas: 122560-63-8) was added in the solution. The solution was filtered and the filtrate was concentrated under reduced pressure to afford racemic 6-methyl-3-(trifluoromethyl)-5,7-dihydro-4H-benzothiophen-6-amine (6) (170 mg, 625.62 μmol, 83.93% yield).

### Procedure for preparation of 6-methyl-3-(trifluoromethyl)-5,7-dihydro-4H-benzothiophen-6-amine hydrochloride enantiomer 1 (241) and enantiomer 2 (242)

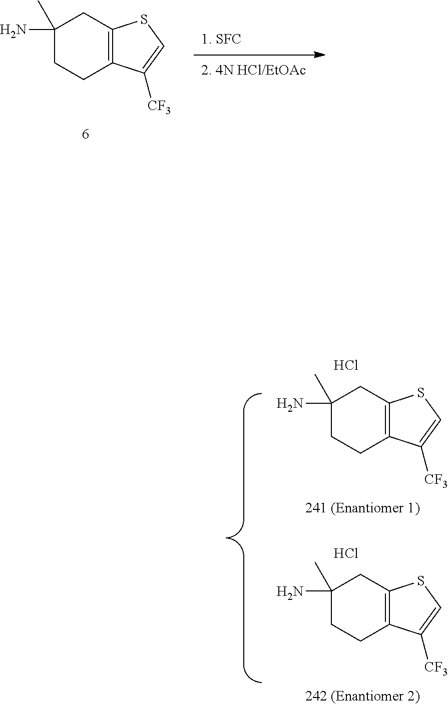

Racemic 6-methyl-3-(trifluoromethyl)-5,7-dihydro-4H-benzothiophen-6-amine (6) was separated to its enantiomers by SFC (column: DAICEL CHIRALPAK IC (250 mm*50 mm, 10 μm); mobile phase: [CO₂-i-PrOH (0.1% NH₃H₂O)]; B %:25%, isocratic elution mode). Enantiomer 1 was 1^{st} peak in SFC (Rt=1.210 min), and enantiomer 2 was 2nd peak in SFC (Rt=1.422 min). The 6-methyl-3-(trifluoromethyl)-5,7-dihydro-4H-benzothiophen-6-amine enantiomer 2 was further purified by prep-HPLC (column: Waters Xbridge 150*25 mm*5 μm; mobile phase: [water(NH₄HCO₃)-ACN]; gradient:28%-58% B over 9 min). The 6-methyl-3-(trifluoromethyl)-5,7-dihydro-4H-benzothiophen-6-amine enantiomer 1 was purified again by SFC (column: DAICEL CHIRALPAK IC (250 mm*50 mm, 10 μm); mobile phase: [CO₂-i-PrOH (0.1% NH₃H₂O)]; B %:25%, isocratic elution mode). Both purified enantiomers were then treated with 4N HCl in EtOAc to obtain the corresponding HCl salts. Compound 6-methyl-3-(trifluoromethyl)-5,7-dihydro-4H-benzothiophen-6-amine hydrochloride enantiomer 1 (241) (63.41 mg, 233.15 μmol, 32.27% yield) was obtained as a white solid. LC-MS: m/z [M+H]⁺=236.0. ¹H NMR (400 MHz, METHANOL-d₄) δ=7.87 (s, 1H), 3.04 (d, J=6.5 Hz, 2H), 2.87 (t, J=6.6 Hz, 2H), 2.04 (dt, J=3.6, 6.7 Hz, 2H), 1.45 (s, 3H). Compound 6-methyl-3-(trifluoromethyl)-5,7-dihydro-4H-benzothiophen-6-amine hydrochloride enantiomer 2 (242) (12.74 mg, 46.71 μmol, 6.46% yield, 99.62% purity, HCl) was obtained as a white solid. LC-MS: m/z [M+H]+=236.0. ¹H NMR (400 MHz, METHANOL-d₄) δ=7.87 (s, 1H), 3.04 (br d, J=6.8 Hz, 2H), 2.86 (br t, J=6.9 Hz, 2H), 2.09-1.99 (m, 2H), 1.44 (s, 3H).

### Synthetic scheme for preparation of 3-(trifluoromethyl)-4,5,6,7-tetrahydrobenzothiophen-6-amine hydrochloride enantiomer 1 (243) and enantiomer 2 (244)

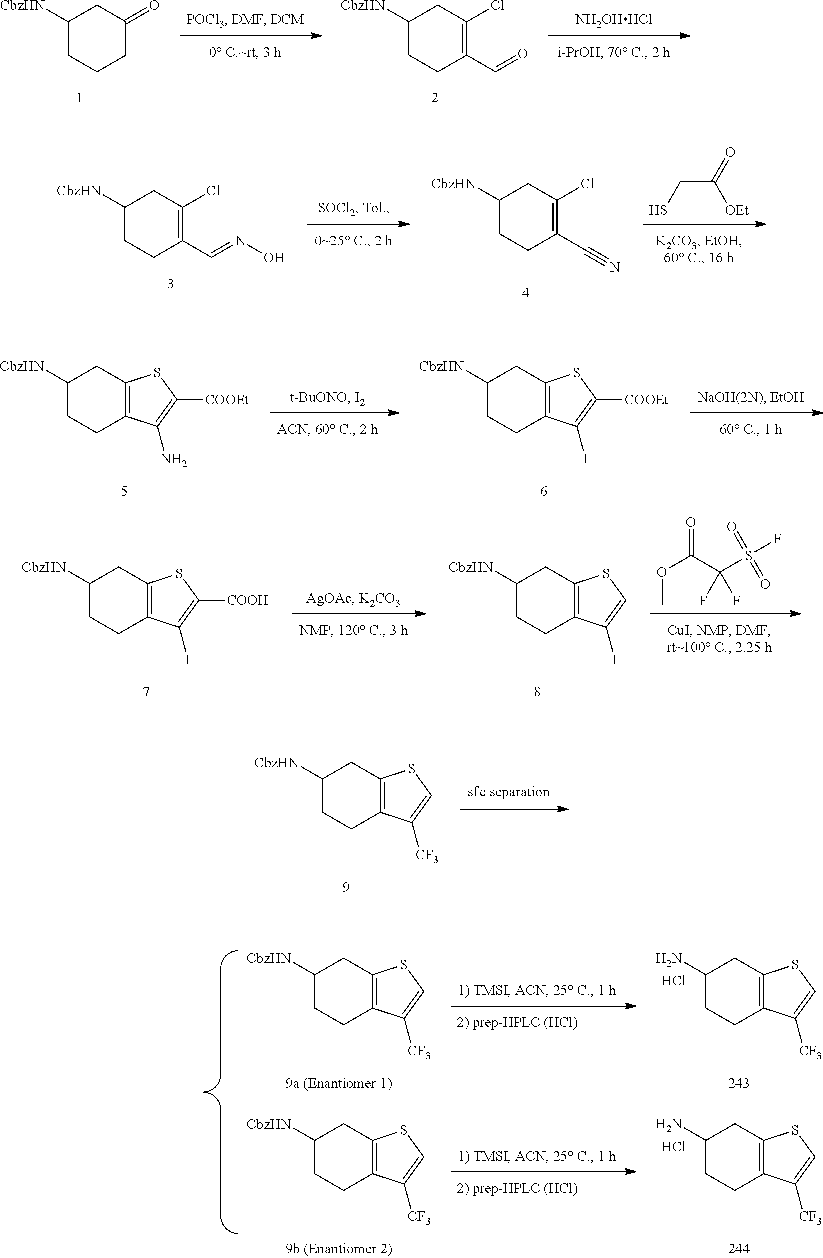

### Procedure for preparation of benzyl N-(3-chloro-4-formyl-cyclohex-3-en-1-yl)carbamate (2)

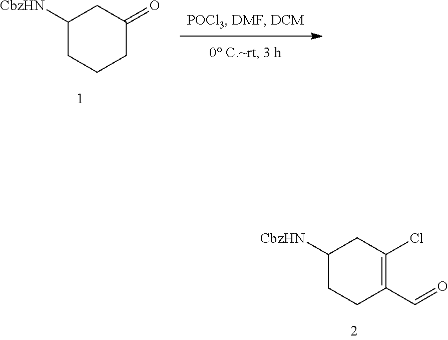 [SMILES: O=C1CCCC(NC(=O)OCc2ccccc2)C1 ; O=CC1=C(Cl)CC(NC(=O)OCc2ccccc2)CC1]

To a solution of DMF (1.48 g, 20.22 mmol, 1.56 mL, 5 eq) in DCM (10 mL) was added POCl₃ (2.48 g, 16.18 mmol, 1.51 mL, 4 eq) slowly at 0° C., the mixture was stirred at 0° C. for 1 hour, then benzyl N-(3-oxocyclohexyl)carbamate (1) (1 g, 4.04 mmol, 1 eq) in DCM (10 mL) was added, the allowed to naturally warm up to 20° C. and stirred for 2 hours. The reaction solution was slowly added to aq. sat. NaHCO₃ while maintaining the pH to 7-8. the mixture was extracted with EtOAc (20 mL*2), the combined organic layer washed with brine (20 mL), dried over anhydrous Na₂SO₄, filtered and concentrated. The residue was purified by flash silica gel chromatography (ISCO®; 12 g SepaFlash® Silica Flash Column, eluent 0~27% Ethyl acetate/Petroleum ether gradient @40 mL/min) to afford benzyl N-(3-chloro-4-formyl-cyclohex-3-en-1-yl)carbamate (2) (460 mg, 1.57 mmol, 38.73% yield) as yellow oil. ¹H NMR (400 MHz, CHLOROFORM-d) δ=10.20-10.16 (m, 1H), 7.41-7.30 (m, 5H), 5.12 (s, 2H), 4.85-4.73 (m, 1H), 4.06-3.93 (m, 1H), 3.01 (br dd, J=4.3, 18.6 Hz, 1H), 2.67-2.42 (m, 2H), 2.40-2.25 (m, 1H), 2.02-1.89 (m, 1H), 1.63 (br s, 1H).

### Procedure for preparation of benzyl N-[3-chloro-4-[(E)-hydroxyiminomethyl]cyclohex-3-en-1-yl]carbamate (3)

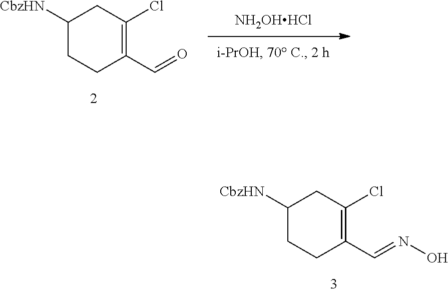 [SMILES: O=CC1=C(Cl)CC(NC(=O)OCc2ccccc2)CC1 ; "NH2OH·HCl" ; O=C(NC1CCC(/C=N/O)=C(Cl)C1)OCc1ccccc1]

To a solution of benzyl N-(3-chloro-4-formyl-cyclohex-3-en-1-yl)carbamate (2) (460 mg, 1.57 mmol, 1 eq) in i-PrOH (5 mL) was added NH₂OH·HCl (119.70 mg, 1.72 mmol, 1.1 eq), the mixture was stirred at 70° C. for 2 hr and then cooled and concentrated to give benzyl N-[3-chloro-4-[(E)-hydroxyiminomethyl]cyclohex-3-en-1-yl]carbamate (3) (550 mg, crude) as a yellow solid.

### Procedure for preparation of benzyl N-(3-chloro-4-cyano-cyclohex-3-en-1-yl)carbamate (4)

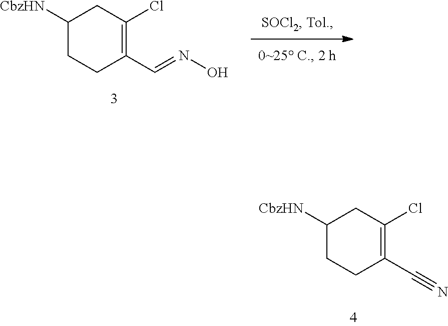 [SMILES: O=C(NC1CCC(/C=N/O)=C(Cl)C1)OCc1ccccc1 ; "SOCl2, Tol.," ; N#CC1=C(Cl)CC(NC(=O)OCc2ccccc2)CC1]

To a solution of benzyl N-[3-chloro-4-[(E)-hydroxyiminomethyl]cyclohex-3-en-1-yl]carbamate (3) (550 mg, 1.78 mmol, 1 eq) in Tol. (5 mL) was added SOCl₂ (635.77 mg, 5.34 mmol, 388.14 μL, 3 eq) at 0° C. The mixture was stirred at 0-25° C. for 2 hr. The reaction solution was slowly added to aq. sat.NaHCO₃ and the the pH was adjusted to 6-7. The mixture was then extracted with EtOAc (10 mL*2), the combined organic layer was washed with brine (10 mL), dried over anhydrous Na₂SO₄, filtered and concentrated to give benzyl N-(3-chloro-4-cyano-cyclohex-3-en-1-yl)carbamate (4) (450 mg, crude) as a yellow solid. LC-MS: m/z [M+Na]⁺=313.0

### Procedure for preparation of ethyl 3-amino-6-(benzyloxycarbonylamino)-4,5,6,7-tetrahydrobenzothiophene-2-carboxylate (5)

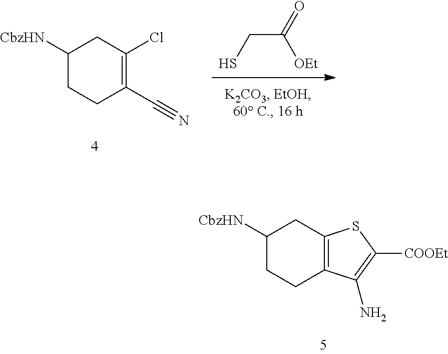 [SMILES: N#CC1=C(Cl)CC(NC(=O)OCc2ccccc2)CC1 ; CCOC(=O)CS ; CCOC(=O)c1sc2c(c1N)CCC(NC(=O)OCc1ccccc1)C2]

To a solution of benzyl N-(3-chloro-4-cyano-cyclohex-3-en-1-yl)carbamate (4) (450 mg, 1.55 mmol, 1 eq) in EtOH (8 mL) was added K₂CO₃ (641.72 mg, 4.64 mmol, 3 eq) and ethyl 2-sulfanylacetate (223.19 mg, 1.86 mmol, 203.64 μL, 1.2 eq). The mixture was stirred at 60° C. for 16 hr. The reaction mixture was concentrated under reduced pressure to remove EtOH. The residue was purified by flash silica gel chromatography (ISCO®; 4 g SepaFlash® Silica Flash Column, eluent 0~25% Ethyl acetate/Petroleum ether gradient @20 mL/min) to afford ethyl 3-amino-6-(benzyloxycarbonylamino)-4,5,6,7-tetrahydrobenzothiophene-2-carboxylate (5) (420 mg, 1.12 mmol, 72.47% yield) as yellow oil. ¹H NMR (400 MHz, CHLOROFORM-d) δ=7.40-7.34 (m, 5H), 5.35 (br s, 2H), 5.17-5.06 (m, 2H), 4.90-4.77 (m, 1H), 4.28 (q, J=7.0 Hz, 2H), 3.13-3.00 (m, 1H), 2.62 (br dd, J=6.8, 16.8 Hz, 1H), 2.50-2.37 (m, 2H), 2.05-1.99 (m, 1H), 1.94-1.83 (m, 1H), 1.37-1.31 (m, 3H). LC-MS: m/z [M+Na]⁺=375.1

### Procedure for preparation of 6-(benzyloxycarbonylamino)-3-iodo-4,5,6,7-tetrahydrobenzothiophene-2-carboxylate (6)

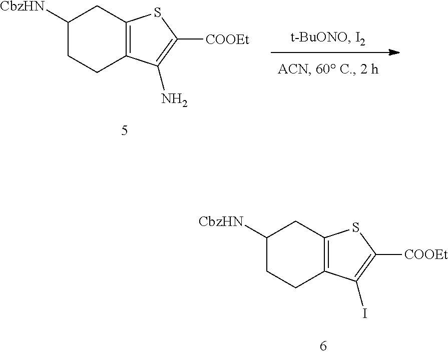 [SMILES: CCOC(=O)c1sc2c(c1N)CCC(NC(=O)OCc1ccccc1)C2 ; CCOC(=O)c1sc2c(c1I)CCC(NC(=O)OCc1ccccc1)C2]

To a solution of ethyl 3-amino-6-(benzyloxycarbonylamino)-4,5,6,7-tetrahydrobenzothiophene-2-carboxylate (5) (420 mg, 1.12 mmol, 1 eq) in ACN (10 mL) was added tert-butyl nitrite (173.49 mg, 1.68 mmol, 200.11 μL, 1.5 eq) and I₂ (341.62 mg, 1.35 mmol, 271.12 μL, 1.2 eq). The mixture was stirred at 60° C. for 2 hr. The reaction mixture was then cooled and poured into H₂O (20 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~20% Ethyl acetate/Petroleum ether gradient @20 mL/min) to afford ethyl 6-(benzyloxycarbonylamino)-3-iodo-4,5,6,7-tetrahydrobenzothiophene-2-carboxylate (6) (340 mg, 700.55 μmol, 62.46% yield) as a yellow solid. ¹H NMR (400 MHz, CHLOROFORM-d) δ=7.44-7.30 (m, 5H), 5.11 (s, 2H), 4.89-4.78 (m, 1H), 4.41-4.30 (m, 2H), 4.21-4.14 (m, 1H), 3.26-3.14 (m, 1H), 2.78-2.63 (m, 3H), 2.11-2.05 (m, 1H), 1.96-1.82 (m, 1H), 1.42-1.35 (m, 3H). LC-MS: m/z [M+Na]⁺=507.9.

### Procedure for preparation of 6-(benzyloxycarbonylamino)-3-iodo-4,5,6,7-tetrahydrobenzothiophene-2-carboxylic acid (7)

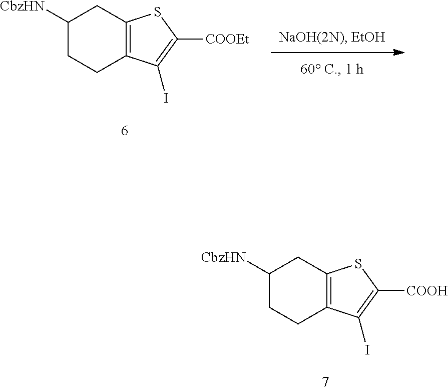 [SMILES: CCOC(=O)c1sc2c(c1I)CCC(NC(=O)OCc1ccccc1)C2 ; O=C(NC1CCc2c(sc(C(=O)O)c2I)C1)OCc1ccccc1]

To a solution of ethyl 6-(benzyloxycarbonylamino)-3-iodo-4,5,6,7-tetrahydrobenzothiophene-2-carboxylate (6) (340 mg, 700.55 μmol, 1 eq) in EtOH (4 mL) was added NaOH (112.08 mg, 2.80 mmol, 1.40 mL, 4 eq) and H₂O (3 mL). The mixture was stirred at 60° C. for 1 hr. The reaction mixture was then cooled and concentrated under reduced pressure to remove EtOH. 1 M HCl aqueous solution was added into the mixture to adjust the pH to 5. The solid formed was filtered and, the filter cake was dried to afford 6-(benzyloxycarbonylamino)-3-iodo-4,5,6,7-tetrahydrobenzothiophene-2-carboxylic acid (7) (260 mg, 568.58 μmol, 81.16% yield) as a yellow solid. ¹H NMR (400 MHz, CHLOROFORM-d) δ=7.45-7.30 (m, 5H), 5.18-5.07 (m, 2H), 4.94-4.80 (m, 1H), 4.23-4.10 (m, 1H), 3.26-3.17 (m, 1H), 2.81-2.74 (m, 1H), 2.71-2.63 (m, 2H), 2.11-2.02 (m, 1H), 1.94-1.84 (m, 1H). LC-MS: m/z [M+Na]⁺=479.9

### Procedure for preparation of benzyl N-(3-iodo-4,5,6,7-tetrahydrobenzothiophen-6-yl)carbamate (8)

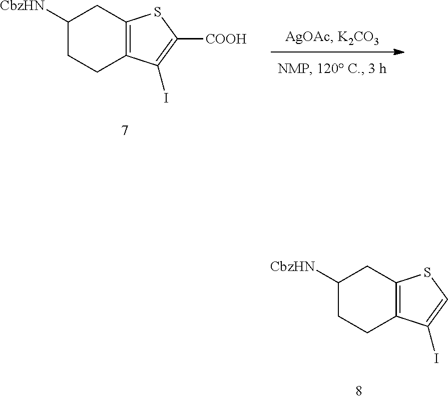 [SMILES: O=C(NC1CCc2c(sc(C(=O)O)c2I)C1)OCc1ccccc1 ; O=C(NC1CCc2c(I)csc2C1)OCc1ccccc1]

To a solution of 6-(benzyloxycarbonylamino)-3-iodo-4,5,6,7-tetrahydrobenzothiophene-2-carboxylic acid (7) (260 mg, 568.58 μmol, 1 eq) in NMP (4 mL) was added AgOAc (18.98 mg, 113.72 μmol, 0.2 eq) and K₂CO₃ (235.74 mg, 1.71 mmol, 3 eq). The mixture was stirred at 120° C. for 3 hr. The reaction mixture was poured into H₂O (10 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~18% Ethyl acetate/Petroleum ether gradient @20 mL/min) to afford benzyl N-(3-iodo-4,5,6,7-tetrahydrobenzothiophen-6-yl)carbamate (8) (200 mg, 483.94 μmol, 85.11% yield) as yellow oil. ¹H NMR (400 MHz, CHLOROFORM-d) δ=7.43-7.33 (m, 6H), 5.19-5.07 (m, 2H), 4.22-4.11 (m, 1H), 3.22-3.12 (m, 1H), 2.72 (dt, J=5.4, 11.3 Hz, 1H), 2.63-2.54 (m, 2H), 1.96-1.84 (m, 1H), 1.81-1.71 (m, 2H)

### Procedure for preparation of benzyl N-[3-(trifluoromethyl)-4,5,6,7-tetrahydrobenzothiophen-6-yl]carbamate (9)

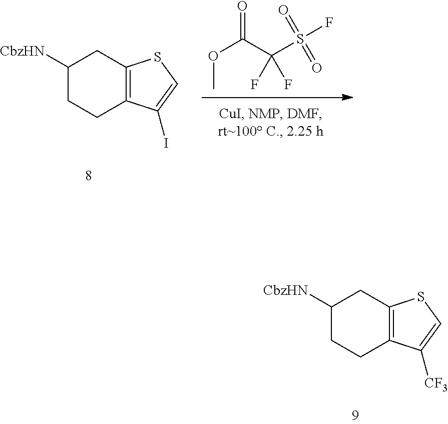

To a solution of benzyl N-(3-iodo-4,5,6,7-tetrahydrobenzothiophen-6-yl)carbamate (8) (200 mg, 483.94 μmol, 1 eq) in DMF (5 mL) was added CuI (101.38 mg, 532.34 μmol, 1.1 eq) and NMP (239.87 mg, 2.42 mmol, 235.17 μL, 5 eq) at 25° C. The mixture was stirred at this temperature for 15 min, and then methyl 2,2-difluoro-2-fluorosulfonyl-acetate (464.86 mg, 2.42 mmol, 307.85 μL, 5 eq) was added dropwise at 25° C. The resulting mixture was stirred at 100° C. for 2 hr. The reaction mixture was then cooled and poured into H₂O (10 mL) and extracted with EtOAc (20 mL*2). The combined organic phases were washed with brine (10 mL), dried over anhydrous sodium sulfate, filtered and concentrated under vacuum to give a residue. The residue was purified by prep-HPLC (column: Phenomenex luna C18 150*25 mm*10 μm; mobile phase: [water(TFA)-ACN]; gradient:60%-90% B over min) to afford benzyl N-[3-(trifluoromethyl)-4,5,6,7-tetrahydrobenzothiophen-6-yl]carbamate (9) (100 mg, 281.39 μmol, 58.15% yield) as a yellow solid. ¹H NMR (400 MHz, METHANOL-d4) δ=7.72 (s, 1H), 7.40-7.26 (m, 5H), 5.09 (s, 2H), 3.99-3.87 (m, 1H), 3.12 (br dd, J=4.7, 16.1 Hz, 1H), 2.91-2.62 (m, 3H), 2.12-2.03 (m, 1H), 1.87-1.73 (m, 1H)

### Procedure for preparation of benzyl N-[3-(trifluoromethyl)-4,5,6,7-tetrahydrobenzothiophen-6-yl]carbamate enantiomer 1 (9a) and enantiomer 2 (9b)

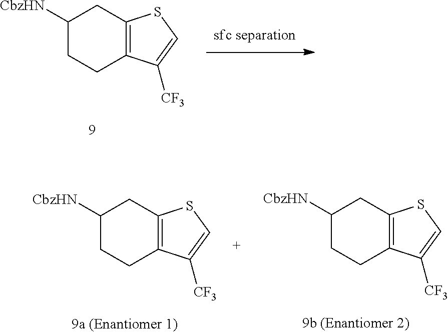 [SMILES: O=C(NC1CCc2c(C(F)(F)F)csc2C1)OCc1ccccc1 ; O=C(NC1CCc2c(C(F)(F)F)csc2C1)OCc1ccccc1 ; O=C(NC1CCc2c(C(F)(F)F)csc2C1)OCc1ccccc1]

Racemic benzyl N-[3-(trifluoromethyl)-4,5,6,7-tetrahydrobenzothiophen-6-yl]carbamate (9) was separated by SFC (column: DAICEL CHIRALPAK AD (250 mm*30 mm, 10 μm); mobile phase: [CO₂-EtOH (0.1% NH₃H₂O)]; B %:15%, isocratic elution mode) to afford benzyl N-[3-(trifluoromethyl)-4,5,6,7-tetrahydrobenzothiophen-6-yl]carbamate enantiomer 1 (9a) (45 mg, 126.63 μmol, 45.00% yield) as a white solid and benzyl N-[3-(trifluoromethyl)-4,5,6,7-tetrahydrobenzothiophen-6-yl]carbamate enantiomer 2 (9b) (45 mg, 126.63 μmol, 45.00% yield) as a white solid.

### Procedure for preparation of 3-(trifluoromethyl)-4,5,6,7-tetrahydrobenzothiophen-6-amine hydrochloride enantiomer 1 (243)

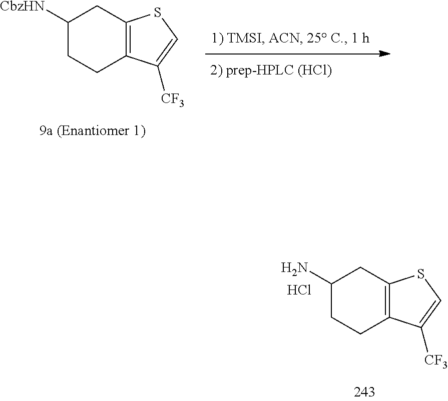 [SMILES: O=C(NC1CCc2c(C(F)(F)F)csc2C1)OCc1ccccc1 ; Cl ; NC1CCc2c(C(F)(F)F)csc2C1]

To a solution of benzyl benzyl N-[3-(trifluoromethyl)-4,5,6,7-tetrahydrobenzothiophen-6-yl]carbamate enantiomer 1 (9a) (45 mg, 126.63 μmol, 1 eq) in ACN (1 mL) was added TMSI (101.35 mg, 506.51 μmol, 68.94 μL, 4 eq). The mixture was stirred at 25° C. for 1 hr. The reaction mixture was then concentrated. The residue was purified by prep-HPLC (column: Phenomenex luna C18 150*25 mm*10 μm; mobile phase: [water(HCl)-ACN]; gradient:1%-30% B over 10 min) to afford 3-(trifluoromethyl)-4,5,6,7-tetrahydrobenzothiophen-6-amine hydrochloride enantiomer 1 (243) (26.79 mg, 102.92 μmol, 81.28% yield) as a white solid. ¹H NMR (400 MHz, METHANOL-d4) δ=7.84 (s, 1H), 3.75-3.60 (m, 1H), 3.25 (br s, 1H), 2.99-2.77 (m, 3H), 2.30-2.18 (m, 1H), 2.03-1.86 (m, 1H). LC-MS: m/z [M+Na]⁺=222.0

### Procedure for preparation of 3-(trifluoromethyl)-4,5,6,7-tetrahydrobenzothiophen-6-amine hydrochloride enantiomer 2 (244)

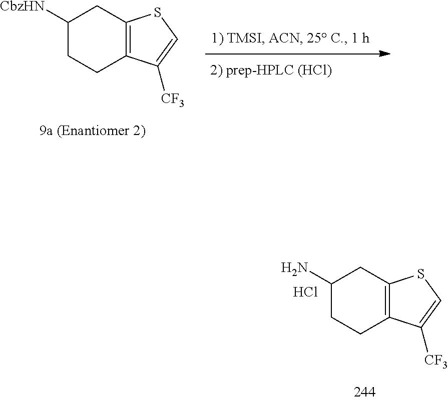 [SMILES: O=C(NC1CCc2c(C(F)(F)F)csc2C1)OCc1ccccc1 ; Cl ; NC1CCc2c(C(F)(F)F)csc2C1]

To a solution of benzyl N-[3-(trifluoromethyl)-4,5,6,7-tetrahydrobenzothiophen-6-yl]carbamate enantiomer 2 (9b) (45 mg, 126.63 μmol, 1 eq) in ACN (1 mL) was added TMSI (101.35 mg, 506.51 μmol, 68.94 μL, 4 eq). The mixture was stirred at 25° C. for 1 hr. The mixture was then concentrated. The residue was purified by prep-HPLC (column: Phenomenex luna C18 150*25 mm*10 μm; mobile phase: [water(HCl)-ACN]; gradient:1%-30% B over 10 min) to afford 3-(trifluoromethyl)-4,5,6,7-tetrahydrobenzothiophen-6-amine hydrochloride enantiomer 2 (244) (29.16 mg, 112.02 μmol, 88.47% yield) as a white solid. ¹H NMR (400 MHz, METHANOL-d4) δ=7.84 (s, 1H), 3.73-3.61 (m, 1H), 3.25 (br s, 1H), 2.98-2.77 (m, 3H), 2.31-2.18 (m, 1H), 2.01-1.87 (m, 1H). LC-MS: m/z [M+Na]⁺=222.0

### Synthetic scheme for synthesis of N, 6-dimethyl-2-(trifluoromethyl)-5,7-dihydro-4H-benzothiophen-6-amine hydrochloride enantiomer 1 (245) and enantiomer 2 (246)

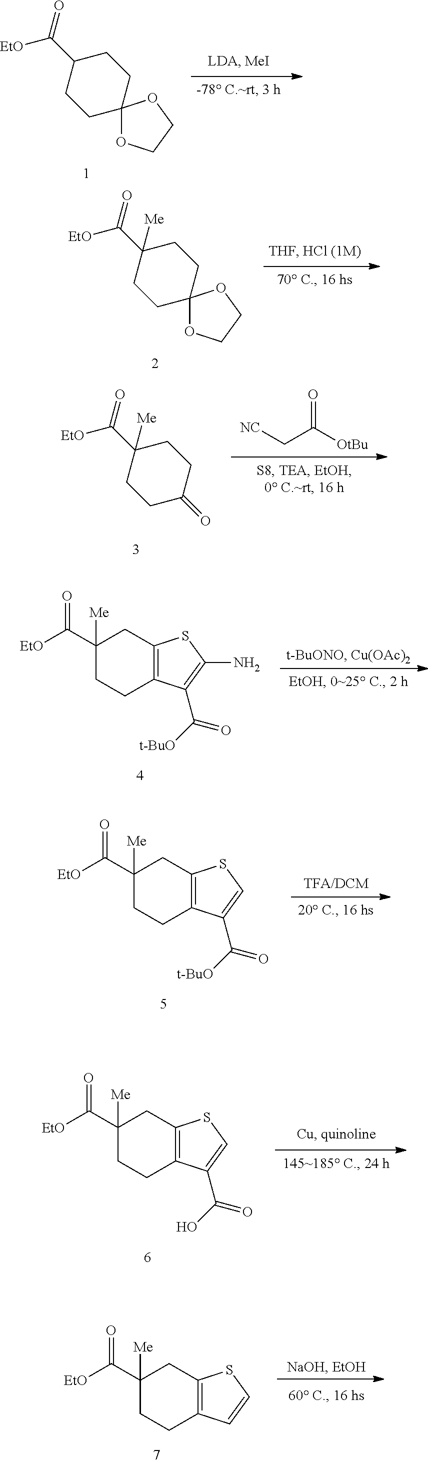

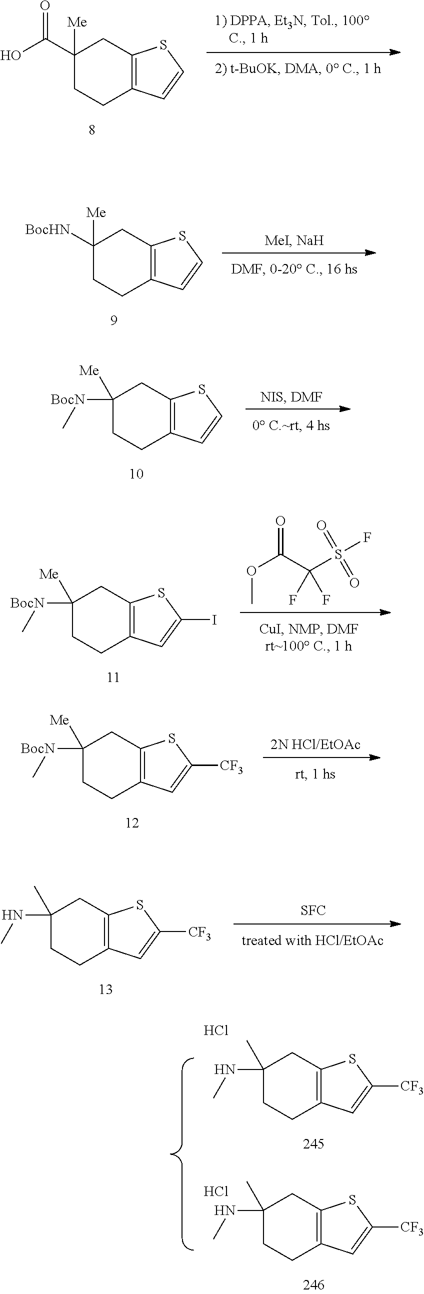

### Procedure for preparation of ethyl 8-methyl-1,4-dioxaspiro[4.5]decane-8-carboxylate (2)

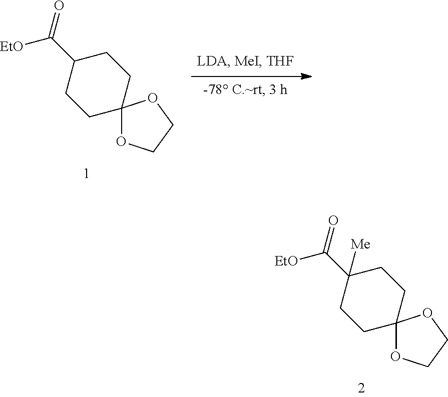

To a solution of LDA (2 M, 280.04 mL, 1.2 eq) in THF (600 mL) was added a solution of ethyl 1,4-dioxaspiro[4.5]decane-8-carboxylate (1) (100 g, 466.73 mmol, 1 eq) in THF (50 mL) dropwise at −78° C. under N₂ and the mixture was stirred at −78° C. for 1 h. A solution of MeI (86.12 g, 606.75 mmol, 37.77 mL, 1.3 eq) in THF (50 mL) was added dropwise next. The mixture was stirred at −78° C. for 1 h, then was warmed to 0° C. and stirred for 1 hr more under N₂. The reaction mixture was then quenched by addition of sat. aq.NH₄Cl 200 mL at 0° C., and diluted with H₂O (200 mL) and extracted with EtOAc (100 mL*3). The combined organic layers were washed with aq. sat. NaCl (180 mL), dried over anhydrous Na₂SO₄, filtered and concentrated under reduced pressure. Compound ethyl 8-methyl-1,4-dioxaspiro[4.5]decane-8-carboxylate (2) (101 g, 442.43 mmol, 94.79% yield) was obtained as a yellow oil. ¹H NMR (400 MHz, CHLOROFORM-d) δ=4.14 (q, J=7.1 Hz, 2H), 3.92 (s, 4H), 2.18-2.08 (m, 2H), 1.69-1.55 (m, 4H), 1.55-1.45 (m, 2H), 1.27-1.21 (m, 3H), 1.18 (s, 3H).

### Procedure for preparation of ethyl 1-methyl-4-oxo-cyclohexanecarboxylate (3)

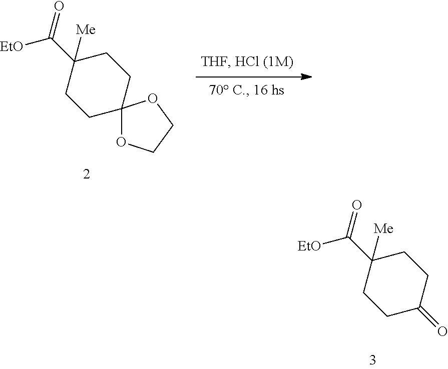 [SMILES: CCOC(=O)C1(C)CCC2(CC1)OCCO2 ; "70° C., 16 hs" ; "THF, HCl (1M)" ; CCOC(=O)C1(C)CCC(=O)CC1]

To a solution of ethyl 8-methyl-1,4-dioxaspiro[4.5]decane-8-carboxylate (2) (101 g, 442.43 mmol, 1 eq) in THF (750 mL) was added aq. HCl (1 M, 1010 mL, 2.28 eq) and the mixture was stirred at 70° C. for 16 hr. The reaction was cooled to r.t., basified by sat. aq. NaHCO₃ to pH 7-8, and extracted with EtOAc (300 mL*2). The organic layers were combined and washed with brine (300 mL), dried over anhydrous Na₂SO₄, filtered and concentrated. Compound ethyl 1-methyl-4-oxo-cyclohexanecarboxylate (3) (78 g, 423.38 mmol, 95.69% yield) was obtained as a yellow oil. ¹H NMR (400 MHz, CHLOROFORM-d) δ=4.22 (q, J=7.1 Hz, 2H), 2.52-2.23 (m, 6H), 1.77-1.57 (m, 2H), 1.37-1.16 (m, 6H).

### Procedure for preparation of 3-tert-butyl 6-ethyl 2-amino-6-methyl-5,7-dihydro-4H-benzothiophene-3,6-dicarboxylate (4)

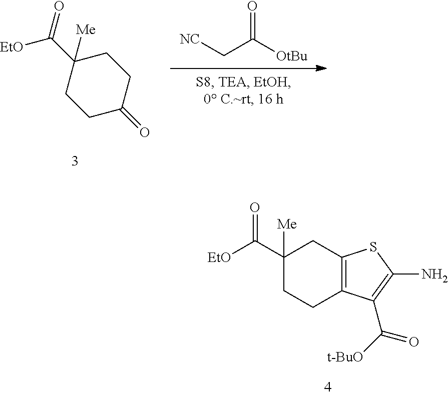

To a solution of ethyl 1-methyl-4-oxo-cyclohexanecarboxylate (3) (78 g, 423.38 mmol, 1 eq) in EtOH (800 mL) was added S8 (24.88 g, 775.92 mmol, 1.83 eq) at 0° C. and tert-butyl 2-cyanoacetate (71.72 g, 508.06 mmol, 72.67 mL, 1.2 eq). The resulting suspension was treated with TEA (85.68 g, 846.76 mmol, 117.86 mL, 2 eq) (maintaining internal temperature below 10° C.) and the reaction was stirred at 25° C. for 16 hours. The reaction was then filtered, the filtrate was concentrated to remove most of the EtOH and treated with EtOAc (600 mL). The mixture was washed with sat. aq. citric acid (250 mL), sat. aq. NaHCO₃ (250 mL), brine (200 mL), dried over anhydrous Na₂SO₄, filtered and concentrated. Compound 3-tert-butyl 6-ethyl 2-amino-6-methyl-5,7-dihydro-4H-benzothiophene-3,6-dicarboxylate (4) (135 g, 397.70 mmol, 93.94% yield) was obtained as a yellow solid. ¹H NMR (400 MHz, CHLOROFORM-d) δ=4.20-4.08 (m, 2H), 3.02 (br d, J=15.9 Hz, 1H), 2.81-2.66 (m, 2H), 2.40 (br d, J=16.0 Hz, 1H), 2.07 (td, J=6.3, 13.1 Hz, 1H), 1.72 (td, J=6.6, 13.3 Hz, 1H), 1.55 (s, 9H), 1.32-1.18 (m, 6H)

### Procedure for preparation of 3-tert-butyl 6-ethyl 6-methyl-5,7-dihydro-4H-benzothiophene-3,6-dicarboxylate (5)

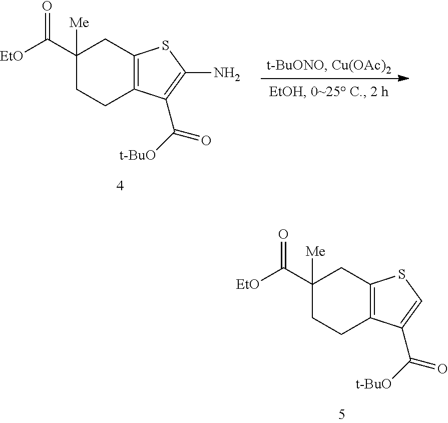

To a solution of 3-tert-butyl 6-ethyl 2-amino-6-methyl-5,7-dihydro-4H-benzothiophene-3,6-dicarboxylate (4) (70 g, 206.22 mmol, 1 eq) in EtOH (1300 mL) was added Cu(OAc)₂ (74.91 g, 412.43 mmol, 2 eq) and t-BuONO (21.27 g, 206.22 mmol, 24.53 mL, 1 eq). The mixture was stirred at 25° C. for 16 hr. The reaction was then filtered, the filtrate was concentrated to remove most of the EtOH, diluted with EtOAc (800 mL) washed with water (500 mL*2), brine (300 mL), dried over anhydrous Na₂SO₄, filtered and concentrated. The residue was purified by column chromatography (SiO₂, Petroleum ether/Ethyl acetate=30/1). Compound 3-tert-butyl 6-ethyl 6-methyl-5,7-dihydro-4H-benzothiophene-3,6-dicarboxylate (5) (62 g, 191.10 mmol, 46.34% yield) was obtained as a yellow oil. ¹H NMR (400 MHz, CHLOROFORM-d) δ=7.84 (s, 1H), 4.13 (q, J=7.1 Hz, 2H), 3.27 (d, J=16.3 Hz, 1H), 2.92 (t, J=6.5 Hz, 2H), 2.63 (d, J=16.3 Hz, 1H), 2.22-2.09 (m, 1H), 1.79 (td, J=6.7, 13.4 Hz, 1H), 1.56 (s, 9H), 1.29 (s, 3H), 1.23 (t, J=7.1 Hz, 3H)

### Procedure for preparation of 6-ethoxycarbonyl-6-methyl-5,7-dihydro-4H-benzothiophene-3-carboxylic acid (6)

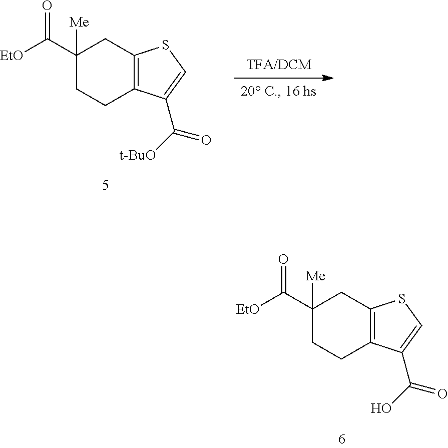 [SMILES: CCOC(=O)C1(C)CCc2c(C(=O)OC(C)(C)C)csc2C1 ; "20° C., 16 hs" ; "TFA/DCM" ; CCOC(=O)C1(C)CCc2c(C(=O)O)csc2C1]

To a solution of 3-tert-butyl 6-ethyl 6-methyl-5,7-dihydro-4H-benzothiophene-3,6-dicarboxylate (5) (62 g, 191.10 mmol, 1 eq) in DCM (1300 mL) was added TFA (199.55 g, 1.75 μmol, 130 mL, 9.16 eq). The mixture was stirred at 20° C. for 16 hr. The reaction was quenched with sat. aq. NaHCO₃ and the pH was maintained to 4-5, the mixture was extracted with DCM (200 mL*2), the organic layers were combined and washed with brine (200 mL), dried over anhydrous Na₂SO₄, filtered and concentrated to get a yellow solid. The crude product was triturated with PE at 20° C. for 30 min. Compound 6-ethoxycarbonyl-6-methyl-5,7-dihydro-4H-benzothiophene-3-carboxylic acid (6) (43.5 g, 162.11 mmol, 84.83% yield) was obtained as a yellow solid. ¹H NMR (400 MHz, CHLOROFORM-d) δ=8.07 (s, 1H), 4.14 (q, J=7.1 Hz, 2H), 3.39-3.21 (m, 1H), 3.05-2.82 (m, 2H), 2.65 (br d, J=16.3 Hz, 1H), 2.24-2.10 (m, 1H), 1.80 (td, J=6.8, 13.6 Hz, 1H), 1.31 (s, 3H), 1.23 (t, J=7.1 Hz, 3H).

### Procedure for preparation of ethyl 6-methyl-5,7-dihydro-4H-benzothiophene-6-carboxylate (7)

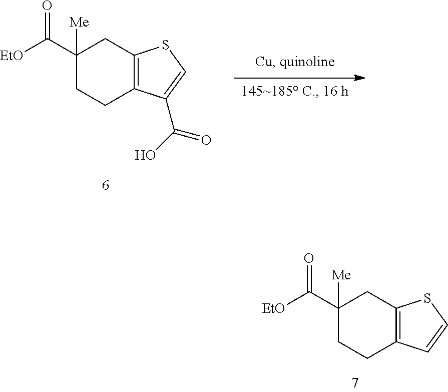

To a solution of 6-ethoxycarbonyl-6-methyl-5,7-dihydro-4H-benzothiophene-3-carboxylic acid (6) (20 g, 74.54 mmol, 1 eq) in quinoline (160 mL) was added Cu (14.21 g, 223.61 mmol, 1.59 mL, 3 eq). The mixture was stirred at 145-185° C. for 16 hr. The reaction was cooled to r.t., filtered and diluted with EtOAc (200 mL), washed with 2 N HCl (200 mL), brine (200 mL), dried over anhydrous Na₂SO₄ filtered and concentrated to get a residue. The residue was purified by column chromatography (SiO₂, Petroleum ether/Ethyl acetate=100 to 1). Compound ethyl 6-methyl-5,7-dihydro-4H-benzothiophene-6-carboxylate (7) (12.5 g, 55.72 mmol, 74.76% yield) was obtained as a yellow oil.

### Procedure for preparation of 6-methyl-5,7-dihydro-4H-benzothiophene-6-carboxylic acid (8)

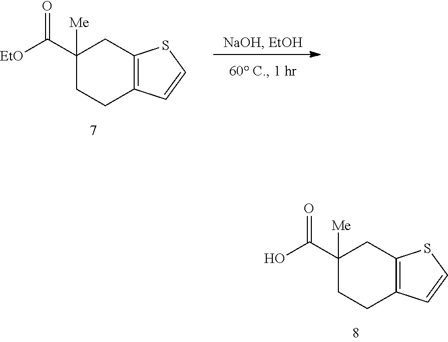

To a solution of ethyl 6-methyl-5,7-dihydro-4H-benzothiophene-6-carboxylate (7) (12.5 g, 55.72 mmol, 1 eq) in EtOH (130 mL) was added NaOH (3 M, 55.72 mL, 3 eq). The mixture was stirred at 60° C. for 1 hr. The reaction was concentrated, diluted with EtOAc (30 mL), acidified with 1 N aq. HCl to pH4~5 and extracted with EtOAc (30 mL). The organic layer was washed with brine (30 mL), dried over anhydrous Na₂SO₄, filtered and concentrated. Compound 6-methyl-5,7-dihydro-4H-benzothiophene-6-carboxylic acid (8) (10.9 g, 51.65 mmol, 92.69% yield) was obtained as a light yellow solid. LC-MS: m/z [M+H]⁺=197.1.

### Procedure for preparation of tert-butyl N-(6-methyl-5,7-dihydro-4H-benzothiophen-6-yl)carbamate (9)

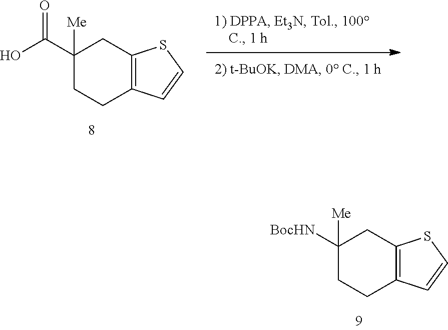

To a solution of 6-methyl-5,7-dihydro-4H-benzothiophene-6-carboxylic acid (8) (9 g, 45.86 mmol, 1 eq) in Tol. (90 mL) was added DPPA (13.88 g, 50.44 mmol, 10.89 mL, 1.1 eq) and TEA (6.96 g, 68.78 mmol, 9.57 mL, 1.5 eq). The mixture was stirred at 100° C. for 1 hr. The reaction mixture was concentrated under vacuum. The residue was dissolved in DMA (90 mL) and treated with t-BuOK (5.66 g, 50.44 mmol, 1.1 eq) at 0° C. Reaction was stirred at 0° C. for 1 hr, quenched by addition of aq. sat.NH₄Cl (80 mL) at 0° C., and then diluted with H₂O (50 mL) and extracted with EtOAc (50 mL*2). The combined organic layers were washed with aq. sat. NaCl (40 mL), dried over anhydrous Na₂SO₄, filtered and concentrated under reduced pressure to give a residue. The residue was purified by flash silica gel chromatography (ISCO®; 20 g SepaFlash® Silica Flash Column, eluent 0~2% Ethyl acetate/Petroleum ether gradient @60 mL/min). Compound tert-butyl N-(6-methyl-5,7-dihydro-4H-benzothiophen-6-yl)carbamate (9) (11.3 g, 42.26 mmol, 92.16% yield) was obtained as a yellow oil. ¹H NMR (400 MHz, CHLOROFORM-d) δ=7.09 (d, J=5.0 Hz, 1H), 6.78 (d, J=5.1 Hz, 1H), 4.51 (br s, 1H), 3.00-2.84 (m, 2H), 2.73-2.56 (m, 2H), 2.46-2.33 (m, 1H), 1.72-1.60 (m, 1H), 1.51-1.39 (m, 12H)

### Procedure for preparation of tert-butyl N-methyl-N-(6-methyl-5,7-dihydro-4H-benzothiophen-6-yl)carbamate (10)

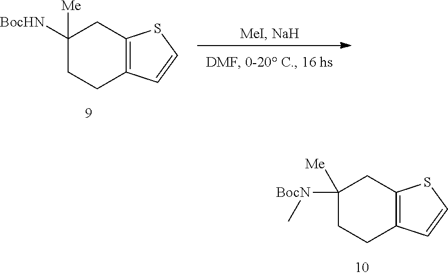

To a solution of tert-butyl N-(6-methyl-5,7-dihydro-4H-benzothiophen-6-yl)carbamate (9) (1 g, 3.74 mmol, 1 eq) in DMF (10 mL) was added NaH (448.75 mg, 11.22 mmol, 60% suspension in mineral oil, 3 eq) and MeI (1.59 g, 11.22 mmol, 698.47 μL, 3 eq) at 0° C. The mixture was allowed to naturally warm up to rt and stirred for 16 hr. The reaction mixture was then quenched with aq. sat.NH₄Cl (40 mL) at 0° C., diluted with H₂O (50 mL) and extracted with EtOAc (50 mL*2). The combined organic layers were washed with aq. sat.NaCl (40 mL), dried over anhydrous Na₂SO₄, filtered and concentrated under reduced pressure. The residue was purified by flash silica gel chromatography (ISCO®; 12 g SepaFlash® Silica Flash Column, eluent 6% Ethyl acetate/Petroleum ether gradient @36 mL/min). Compound tert-butyl N-methyl-N-(6-methyl-5,7-dihydro-4H-benzothiophen-6-yl)carbamate (10) (0.95 g, 3.34 mmol, 89.36% yield) was obtained as a white solid. LC-MS: m/z [M+H-tBu]⁺=182.0.

### Procedure for preparation of tert-butyl N-(2-iodo-6-methyl-5,7-dihydro-4H-benzothiophen-6-yl)-N-methyl-carbamate (11)

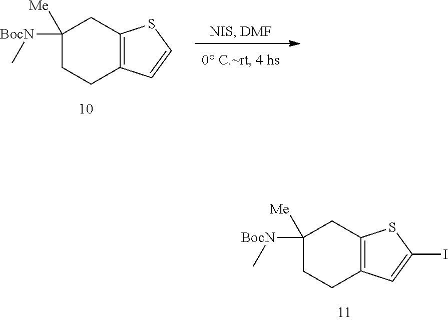 [SMILES: CN(C(=O)OC(C)(C)C)C1(C)CCc2ccsc2C1 ; CN(C(=O)OC(C)(C)C)C1(C)CCc2cc(I)sc2C1]

To a solution of tert-butyl N-methyl-N-(6-methyl-5,7-dihydro-4H-benzothiophen-6-yl)carbamate (10) (500 mg, 1.78 mmol, 1 eq) in DMF (8 mL) was added NIS (519.66 mg, 2.31 mmol, 1.3 eq). The mixture was stirred at rt for 16 hr and then poured into water (100 mL) and extracted with EtOAc (50 mL*3). The combined organic phase was washed with brine (50 mL), dried over anhydrous sodium sulfate, filtered and concentrated under vacuum. The residue was purified by flash silica gel chromatography (ISCO®; 12 g SepaFlash® Silica Flash Column, eluent 0~8% Ethyl acetate/Petroleum ether gradient @50 mL/min). Compound tert-butyl N-(2-iodo-6-methyl-5,7-dihydro-4H-benzothiophen-6-yl)-N-methyl-carbamate (11) (0.6 g, 1.47 mmol, 82.91% yield) was obtained as a white solid. ¹H NMR (400 MHz, CHLOROFORM-d) δ=6.91 (s, 1H), 3.18 (d, J=17.0 Hz, 1H), 2.89-2.71 (m, 5H), 2.58 (br t, J=6.1 Hz, 2H), 1.78-1.68 (m, 1H), 1.46 (s, 9H), 1.41 (s, 3H). LC-MS: m/z [M+H-tBu]⁺=308.1.

### Procedure for preparation of tert-butyl N-methyl-N-[6-methyl-2-(trifluoromethyl)-5,7-dihydro-4H-benzothiophen-6-yl]carbamate (12)

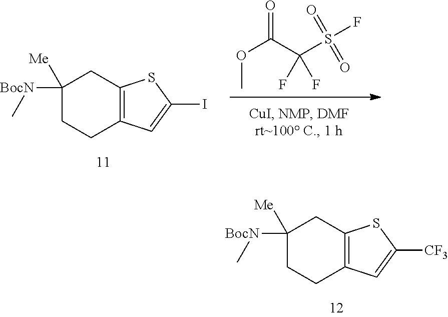

To a solution of tert-butyl N-(2-iodo-6-methyl-5,7-dihydro-4H-benzothiophen-6-yl)-N-methyl-carbamate (11) (0.6 g, 1.47 mmol, 1 eq) in DMF (7 mL) was added methyl 2,2-difluoro-2-fluorosulfonyl-acetate (1.42 g, 7.37 mmol, 937.09 μL, 5 eq), CuI (1.40 g, 7.37 mmol, 5 eq) and NMP (219.04 mg, 2.21 mmol, 214.75 μL, 1.5 eq). The mixture was stirred at 100° C. for 1 hr. The reaction mixture was then concentrated under reduced pressure diluted with EtOAc 20 mL and filtered through a pad of diatomite. The filtrate was diluted with H₂O (30 mL) and extracted with EtOAc (10 mL*3). The combined organic layers were washed with brine (10 mL*3), dried over anhydrous sodium sulfate, filtered and concentrated under reduced pressure to give a residue. The residue was purified by flash silica gel chromatography (ISCO®; 12 g SepaFlash® Silica Flash Column, eluent 0~10% Ethyl acetate/Petroleum ether gradient @36 mL/min). The residue was purified by prep-HPLC (column: Waters Xbridge 150*25 mm*5 μm; mobile phase: [water(NH₄HCO₃)-ACN]; gradient:70%-100% B over 9 min). Compound tert-butyl N-methyl-N-[6-methyl-2-(trifluoromethyl)-5,7-dihydro-4H-benzothiophen-6-yl]carbamate (12) (230 mg, 658.25 μmol, 44.69% yield) was obtained as a yellow oil. LC-MS: m/z [M+H-tBu]⁺=293.9.

### Procedure for preparation of N, 6-dimethyl-2-(trifluoromethyl)-5,7-dihydro-4H-benzothiophen-6-amine (13)

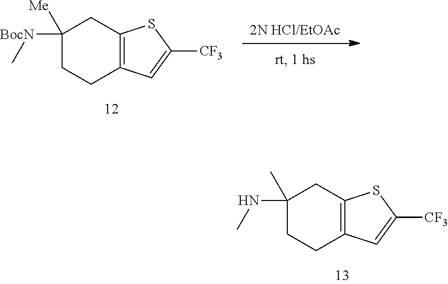 [SMILES: CN(C(=O)OC(C)(C)C)C1(C)CCc2cc(C(F)(F)F)sc2C1 ; "2N HCl/EtOAc" ; CNC1(C)CCc2cc(C(F)(F)F)sc2C1]

To a solution of tert-butyl N-methyl-N-[6-methyl-2-(trifluoromethyl)-5,7-dihydro-4H-benzothiophen-6-yl]carbamate (12) (210 mg, 601.01 μmol, 1 eq) in EtOAc (2 mL) was added HCl/EtOAc (4 M, 4 mL, 26.62 eq). The mixture was stirred at rt for 1 hr and then concentrated under vacuum to give a residue. The residue was diluted with 10 mL MeOH and anion exchange resin (AMBERLITE(R)IRA-400 (CL, cas: 122560-63-8) (1 g) was added in the solution. The solution was stirred and filtered and the filtrate was concentrated under reduced pressure. Compound N, 6-dimethyl-2-(trifluoromethyl)-5,7-dihydro-4H-benzothiophen-6-amine free base (13) (130 mg, 521.47 μmol, 86.77% yield) was obtained as a white solid.

### Procedure for preparation of N, 6-dimethyl-2-(trifluoromethyl)-5,7-dihydro-4H-benzothiophen-6-amine hydrochloride enantiomer 1 (245) and enantiomer 2 (246)

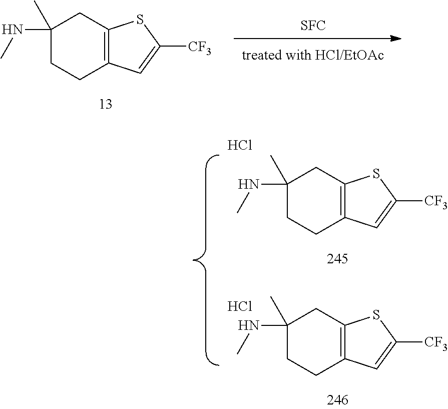 [SMILES: CNC1(C)CCc2cc(C(F)(F)F)sc2C1 ; "SFC" ; Cl ; CNC1(C)CCc2cc(C(F)(F)F)sc2C1 ; Cl ; CNC1(C)CCc2cc(C(F)(F)F)sc2C1]

Racemic N, 6-dimethyl-2-(trifluoromethyl)-5,7-dihydro-4H-benzothiophen-6-amine (13) (130 mg) was separated to its enantiomers by SFC (column: Phenomenex-Cellulose-2 (250 mm*50 mm, 10 μm); mobile phase: [CO₂-EtOH (0.1% NH₃H₂O)]; B %:15%, isocratic elution mode). Enantiomer 1 was1^{st} peak in SFC (Rt=1.257 min) and enantiomer 2 was 2nd peak in SFC, (Rt=1.335 min). To each of the enantiomers was added 4 M HCl/EtOAc (1 mL). Each of the mixtures formed was stirred and then evaporated to afford the corresponding HCl salts. Compound N, 6-dimethyl-2-(trifluoromethyl)-5,7-dihydro-4H-benzothiophen-6-amine hydrochloride enantiomer 1 (245) (50.87 mg, 176.17 μmol, 33.78% yield) was obtained as an orange solid. ¹H NMR (400 MHz, METHANOL-d₄) δ=7.31 (s, 1H), 3.10 (s, 2H), 2.84 (br t, J=6.3 Hz, 2H), 2.72 (s, 3H), 2.06 (t, J=6.6 Hz, 2H), 1.42 (s, 3H). LC-MS: m/z [M+H]⁺=250.2. Compound N, 6-dimethyl-2-(trifluoromethyl)-5,7-dihydro-4H-benzothiophen-6-amine hydrochloride enantiomer 2 (246) (57.80 mg, 202.27 μmol, 38.79% yield) was obtained as an orange solid. ¹H NMR (400 MHz, METHANOL-d₄) δ=7.31 (s, 1H), 3.10 (s, 2H), 2.84 (br t, J=6.2 Hz, 2H), 2.73 (s, 3H), 2.11-2.03 (m, 2H), 1.43 (s, 3H). LC-MS: m/z [M+H]⁺=250.2.

### Synthetic scheme for preparation of N, 3-dimethyl-6,7-dihydro-5H-thieno[3,2-b]pyran-6-amine hydrochloride (247)

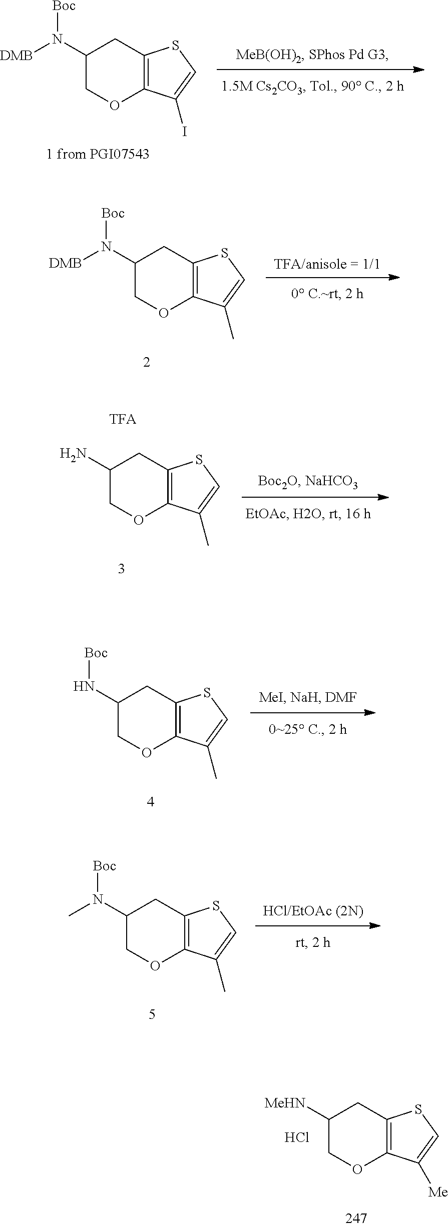

### Procedure for preparation of tert-butyl N-[(2,4-dimethoxyphenyl)methyl]-N-(3-methyl-6,7-dihydro-5H-thieno[3,2-b]pyran-6-yl)carbamate (2)

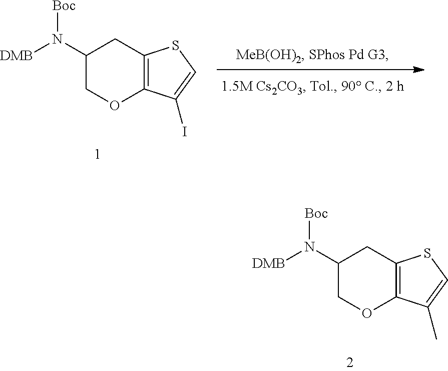

A mixture of tert-butyl N-[(2,4-dimethoxyphenyl)methyl]-N-(3-iodo-6,7-dihydro-5H-thieno[3,2-b]pyran-6-yl)carbamate (1) (200 mg, 376.36 μmol, 1 eq), methylboronic acid (135.17 mg, 2.26 mmol, 6 eq), SPhos Pd G3 (29.37 mg, 37.64 μmol, 0.1 eq), Cs₂CO₃ (1.5 M, 752.72 μL, 3 eq) in Tol. (4 mL) was degassed and purged with N₂ for 3 times, and then the mixture was stirred at 90° C. for 2 hr under N₂ atmosphere. The reaction mixture was poured into H₂O (20 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~20% Ethyl acetate/Petroleum ether gradient @40 mL/min) to afford tert-butyl N-[(2,4-dimethoxyphenyl)methyl]-N-(3-methyl-6,7-dihydro-5H-thieno[3,2-b]pyran-6-yl)carbamate (2) (140 mg, 333.70 μmol, 88.67% yield) as a white solid. LC-MS: m/z [M+Na]⁺=442.1

### Procedure for preparation of 3-methyl-6,7-dihydro-5H-thieno[3,2-b]pyran-6-amine (3)

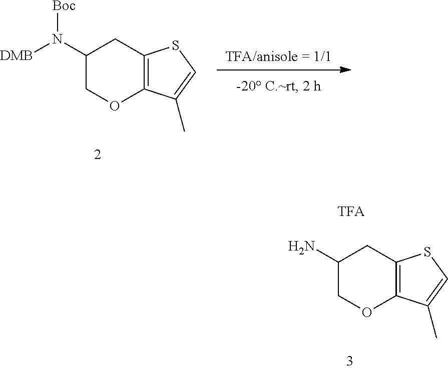

To a solution of tert-butyl N-[(2,4-dimethoxyphenyl)methyl]-N-(3-methyl-6,7-dihydro-5H-thieno[3,2-b]pyran-6-yl)carbamate (2) (140 mg, 333.70 μmol, 1 eq) in anisole (995.00 mg, 9.20 mmol, 1 mL, 27.57 eq) was added TFA (1 mL) at −20° C. The mixture was stirred at 25° C. for 2 hr. The reaction mixture was poured into H₂O (10 mL) and extracted with PE (10 mL*2). The organic phase was separated, washed with H₂O (10 mL*3). The aqueous phase was combined and lyophilized to give 3-methyl-6,7-dihydro-5H-thieno[3,2-b]pyran-6-amine TFA salt (3) (60 mg, crude) as yellow oil.

### Procedure for preparation of tert-butyl N-(3-methyl-6,7-dihydro-5H-thieno[3,2-b]pyran-6-yl)carbamate (4)

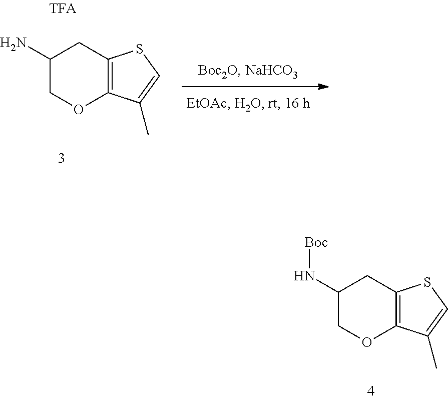

To a solution of 3-methyl-6,7-dihydro-5H-thieno[3,2-b]pyran-6-amine TFA salt (3) (60 mg, 354.52 μmol, 1 eq) in EtOAc (3 mL) and H₂O (3 mL) was added aq. sat NaHCO₃ (297.82 mg, 3.55 mmol, 137.94 μL, 10 eq) and (Boc)₂O (77.37 mg, 354.52 μmol, 81.44 μL, 1 eq). The mixture was stirred at 25° C. for 16 hr. The reaction mixture was poured into H₂O (10 mL) and extracted with EtOAc (10 mL*2). The combined organic phases were washed with brine (10 mL), dried over anhydrous sodium sulfate, filtered and concentrated under vacuum to give a residue that was purified by flash silica gel chromatography (ISCO®; 20 g SepaFlash® Silica Flash Column, eluent 0~9% Ethyl acetate/Petroleum ether gradient @40 mL/min), and then by prep-HPLC (column: Phenomenex Gemini NX C18 150*30 mm*3 μm; mobile phase: [water(FA)-ACN]; gradient:55%-85% B over 7 min) to afford tert-butyl N-(3-methyl-6,7-dihydro-5H-thieno[3,2-b]pyran-6-yl)carbamate (4) (30 mg, 111.38 μmol, 31.42% yield) as yellow oil. LC-MS: m/z [M+H-tBu]⁺=214.0

### Procedure for preparation of tert-butyl N-methyl-N-(3-methyl-6,7-dihydro-5H-thieno[3,2-b]pyran-6-yl)carbamate (5)

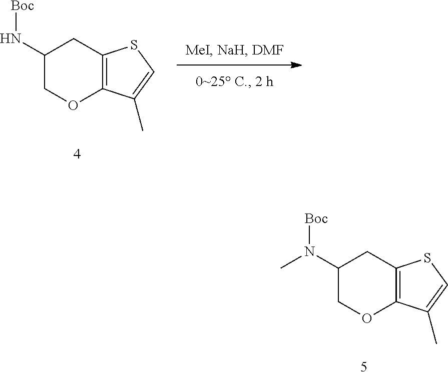

To a solution of tert-butyl N-(3-methyl-6,7-dihydro-5H-thieno[3,2-b]pyran-6-yl)carbamate (4) (30 mg, 111.38 μmol, 1 eq) in DMF (1 mL) was added NaH (8.91 mg, 222.75 μmol, 60% suspension in mineral oil, 2 eq) at 0° C. After addition, the mixture was stirred at this temperature for 0.5 hr, and then CH₃I (23.71 mg, 167.06 μmol, 10.40 μL, 1.5 eq) was added dropwise at 0° C. The resulting mixture was allowed to naturally warm up to room temperature and stirred for 1.5 hr. The reaction mixture was then poured into NH₄Cl aq. saturated solution (5 mL) and extracted with EtOAc (5 mL*3). The combined organic phases were washed with brine (10 mL), dried over anhydrous sodium sulfate, filtered and concentrated under vacuum to give tert-butyl N-methyl-N-(3-methyl-6,7-dihydro-5H-thieno[3,2-b]pyran-6-yl)carbamate (5) (25 mg, crude) as yellow oil. LC-MS: m/z [M+H-tBu]⁺=228.1.

### Procedure for preparation of N, 3-dimethyl-6,7-dihydro-5H-thieno[3,2-b]pyran-6-amine hydrochloride (247)

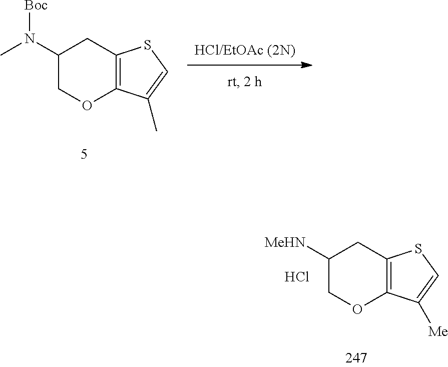 [SMILES: Cc1csc2c1OCC(N(C)C(=O)OC(C)(C)C)C2 ; "HCl/EtOAc (2N)" ; Cl ; CNC1COc2c(C)csc2C1]

To a solution of tert-butyl N-methyl-N-(3-methyl-6,7-dihydro-5H-thieno[3,2-b]pyran-6-yl)carbamate (5) (25 mg, 88.22 μmol, 1 eq) in EtOAc (1 mL) was added HCl/EtOAc (4 M, 1 mL, 45.34 eq). The mixture was stirred at 25° C. for 2 hr. The mixture was concentrated. The residue was purified by prep-HPLC (column: Phenomenex luna C18 150*25 mm*10 μm; mobile phase: [water(HCl)-ACN]; gradient:1%-20% B over 10 min) to afford N, 3-dimethyl-6,7-dihydro-5H-thieno[3,2-b]pyran-6-amine hydrochloride (247) (16.95 mg, 76.37 μmol, 86.57% yield) as a white solid. ¹H NMR (400 MHz, METHANOL-d4) δ=6.85 (s, 1H), 4.64-4.49 (m, 2H), 4.20 (br d, J=12.4 Hz, 1H), 3.83-3.74 (m, 1H), 3.03-2.93 (m, 1H), 2.80 (s, 3H), 2.07 (s, 3H). LC-MS: m/z [M+H]⁺=184.

### Synthetic scheme for preparation of N-methyl-3-(trifluoromethyl)-6,7-dihydro-5H-thieno[3,2-b]pyran-6-amine hydrochloride (248)

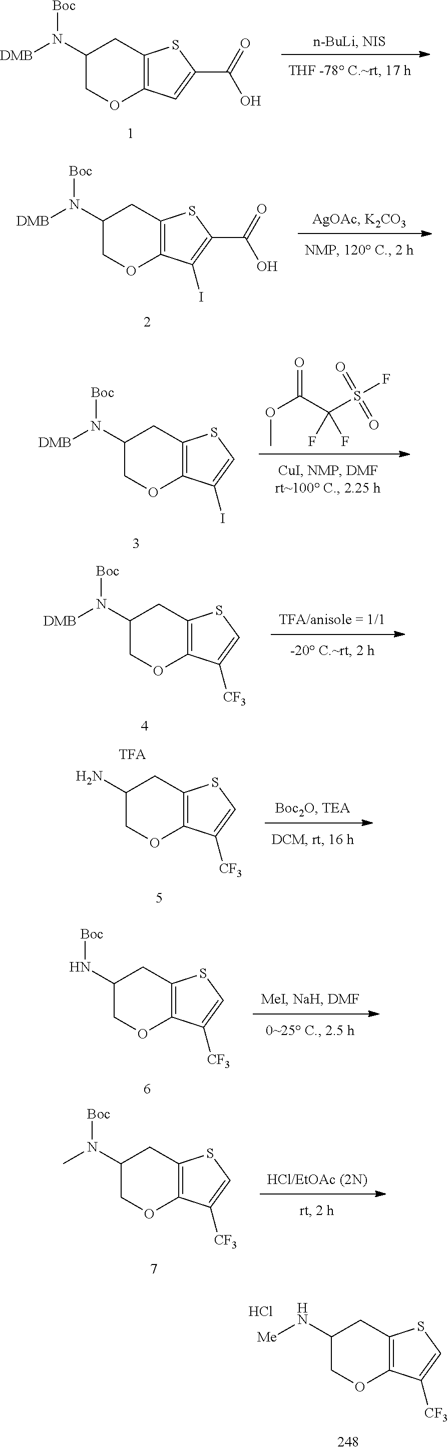

### Procedure for preparation of 6-[tert-butoxycarbonyl-[(2,4-dimethoxyphenyl)methyl]amino]-3-iodo-6,7-dihydro-5H-thieno[3,2-b]pyran-2-carboxylic acid (2)

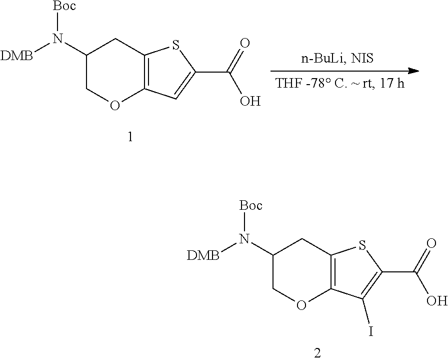

To a solution of 6-[tert-butoxycarbonyl-[(2,4-dimethoxyphenyl)methyl]amino]-6,7-dihydro-5H-thieno[3,2-b]pyran-2-carboxylic acid (1) (1.37 g, 3.05 mmol, 1 eq) in THF (15 mL) was added dropwise n-BuLi (2.5 M, 2.93 mL, 2.4 eq) at −78° C. over 0.5 hr. After addition, the mixture was stirred at this temperature for 0.5 hr, and then NIS (1.03 g, 4.57 mmol, 1.5 eq) in THF (5 mL) was added dropwise at −78° C. The resulting mixture was stirred at −78° C. for 0.5 h and then at rt for 16 hr. The reaction mixture was then poured into NH₄Cl aq. saturated solution (50 mL) and extracted with EtOAc (20 mL*2). The combined organic phases were washed with brine (10 mL), dried over anhydrous sodium sulfate, filtered and concentrated under vacuum to give a residue. The residue was purified by prep-HPLC (column: Phenomenex luna C18 150*40 mm*15 μm; mobile phase: [water(FA)-ACN]; gradient:50%-80% B over 15 min) to afford 6-[tert-butoxycarbonyl-[(2,4-dimethoxyphenyl)methyl]amino]-3-iodo-6,7-dihydro-5H-thieno[3,2-b]pyran-2-carboxylic acid (2) (550 mg, 955.84 μmol, 31.36% yield) as a yellow solid. LC-MS: m/z [M+Na]⁺=598.0

### Procedure for preparation of tert-butyl N-[(2,4-dimethoxyphenyl)methyl]-N-(3-iodo-6,7-dihydro-5H-thieno[3,2-b]pyran-6-yl)carbamate (3)

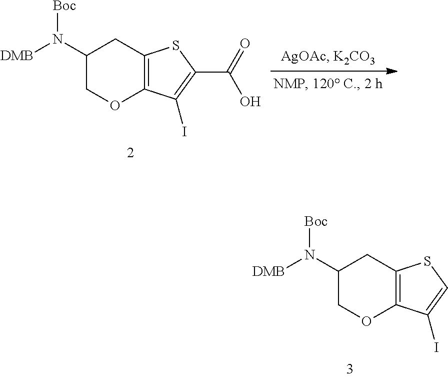

To a solution of 6-[tert-butoxycarbonyl-[(2,4-dimethoxyphenyl)methyl]amino]-3-iodo-6,7-dihydro-5H-thieno[3,2-b]pyran-2-carboxylic acid (2) (550 mg, 955.84 μmol, 1 eq) in NMP (7 mL) was added AgOAc (31.91 mg, 191.17 μmol, 0.2 eq) and K₂CO₃ (396.31 mg, 2.87 mmol, 3 eq). The mixture was stirred at 120° C. for 2 hr. The reaction mixture was then cooled and poured into H₂O (30 mL) and extracted with EtOAc (30 mL*2). The combined organic phases were washed with brine (30 mL), dried over anhydrous sodium sulfate, filtered and concentrated under vacuum to give a residue. The residue was purified by prep-HPLC (column: Phenomenex luna C18 150*40 mm*15 μm; mobile phase: [water(FA)-ACN]; gradient:65%-95% B over 15 min) to afford tert-butyl N-[(2,4-dimethoxyphenyl)methyl]-N-(3-iodo-6,7-dihydro-5H-thieno[3,2-b]pyran-6-yl)carbamate (3) (400 mg, 752.72 μmol, 78.75% yield) as a red solid. LC-MS: m/z [M+Na]⁺=554.1

### Procedure for preparation of tert-butyl N-[(2,4-dimethoxyphenyl)methyl]-N-[3-(trifluoromethyl)-6,7-dihydro-5H-thieno[3,2-b]pyran-6-yl]carbamate (4)

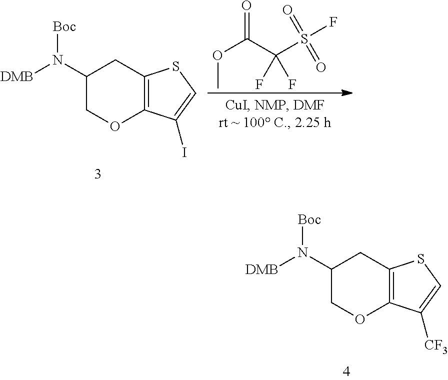

To a solution of tert-butyl N-[(2,4-dimethoxyphenyl)methyl]-N-(3-iodo-6,7-dihydro-5H-thieno[3,2-b]pyran-6-yl)carbamate (3) (200 mg, 376.36 μmol, 1 eq) in DMF (5 mL) was added CuI (78.85 mg, 414.00 μmol, 1.1 eq) and NMP (186.55 mg, 1.88 mmol, 182.89 μL, 5 eq) at 25° C. After addition, the mixture was stirred at this temperature for 15 min, and then methyl 2,2-difluoro-2-fluorosulfonyl-acetate (361.52 mg, 1.88 mmol, 239.42 L, 5 eq) was added dropwise at 25° C. The resulting mixture was stirred at 100° C. for 2 hr. The reaction mixture was then cooled and poured into H₂O (10 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~20% Ethyl acetate/Petroleum ether gradient @40 mL/min) to afford tert-butyl N-[(2,4-dimethoxyphenyl)methyl]-N-[3-(trifluoromethyl)-6,7-dihydro-5H-thieno[3,2-b]pyran-6-yl]carbamate (4) (150 mg, 316.79 μmol, 84.17% yield) as yellow oil. LC-MS: m/z [M+Na]⁺=496.1

### Procedure for preparation of 3-(trifluoromethyl)-6,7-dihydro-5H-thieno[3,2-b]pyran-6-amine (5)

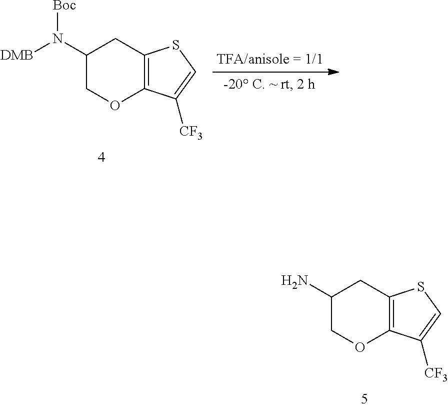

To a solution of tert-butyl N-[(2,4-dimethoxyphenyl)methyl]-N-[3-(trifluoromethyl)-6,7-dihydro-5H-thieno[3,2-b]pyran-6-yl]carbamate (4) (150 mg, 316.79 μmol, 1 eq) in anisole (995.00 mg, 9.20 mmol, 1 mL, 29.05 eq) was added TFA (1 mL) at−20° C. The mixture was allowed to naturally warm up and stirred at 25° C. for 2 hr. The reaction mixture was poured into H₂O (10 mL) and extracted with PE (10 mL*2). The organic phase was separated, washed with H₂O 30 mL (10 mL*3). The aqueous phase was combined and lyophilized to give 3-(trifluoromethyl)-6,7-dihydro-5H-thieno[3,2-b]pyran-6-amine (5) (70 mg, crude) as yellow oil. LC-MS: m/z [M+H]⁺=224.1

### Procedure for preparation of tert-butyl N-[3-(trifluoromethyl)-6,7-dihydro-5H-thieno[3,2-b]pyran-6-yl]carbamate (6)

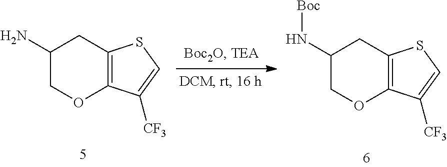 [SMILES: NC1COc2c(C(F)(F)F)csc2C1 ; CC(C)(C)OC(=O)NC1COc2c(C(F)(F)F)csc2C1]

To a solution of 3-(trifluoromethyl)-6,7-dihydro-5H-thieno[3,2-b]pyran-6-amine (5) (70 mg, 313.60 μmol, 1 eq) in H₂O (5 mL) and EtOAc (5 mL) was added aq. saturated NaHCO₃ (131.72 mg, 1.57 mmol, 61.01 μL, 5 eq) and (Boc)₂O (136.88 mg, 627.20 μmol, 144.09 μL, 2 eq). The mixture was stirred at 25° C. for 16 hr. The reaction mixture was then poured into H₂O (5 mL) and extracted with EtOAc (10 mL*2). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~30% Ethyl acetate/Petroleum ether gradient @40 mL/min) to afford tert-butyl N-[3-(trifluoromethyl)-6,7-dihydro-5H-thieno[3,2-b]pyran-6-yl]carbamate (6) (100 mg, 309.28 μmol, 98.62% yield) as yellow oil. ¹H NMR (400 MHz, CHLOROFORM-d) δ=7.46 (s, 1H), 5.03-4.90 (m, 1H), 4.33-4.19 (m, 2H), 4.13-4.08 (m, 1H), 3.07 (br dd, J=4.6, 16.3 Hz, 1H), 2.84-2.70 (m, 1H), 1.46 (s, 9H)

### Procedure for preparation of tert-butyl N-methyl-N-[3-(trifluoromethyl)-6,7-dihydro-5H-thieno[3,2-b]pyran-6-yl]carbamate (7)

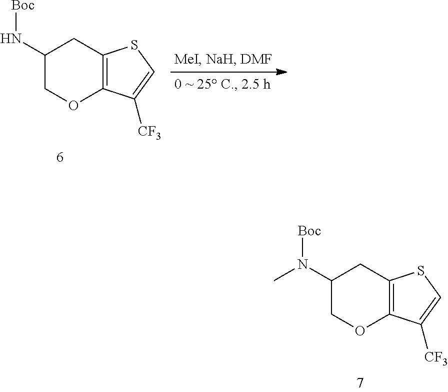 [SMILES: CC(C)(C)OC(=O)NC1COc2c(C(F)(F)F)csc2C1 ; CN(C(=O)OC(C)(C)C)C1COc2c(C(F)(F)F)csc2C1]

To a solution of tert-butyl N-[3-(trifluoromethyl)-6,7-dihydro-5H-thieno[3,2-b]pyran-6-yl]carbamate (6) (100 mg, 309.28 μmol, 1 eq) in DMF (2 mL) was added dropwise NaH (18.56 mg, 463.92 μmol, 60% suspension in mineral oil, 1.5 eq) at 0° C. After addition, the mixture was stirred at this temperature for 0.5 hr, and then CH₃I (65.85 mg, 463.92 μmol, 28.88 μL, 1.5 eq) was added dropwise at 0° C. The resulting mixture was stirred at 25° C. for 2 hr. The reaction mixture was poured into NH₄Cl aq. saturated solution (10 mL) slowly and extracted with EtOAc (10 mL*3). The combined organic phases were washed with brine (10 mL), dried over anhydrous sodium sulfate, filtered and concentrated under vacuum to give a residue. The residue was purified by flash silica gel chromatography (ISCO®; 4 g SepaFlash® Silica Flash Column, eluent 0~14% Ethyl acetate/Petroleum ether gradient @20 mL/min) to afford tert-butyl N-methyl-N-[3-(trifluoromethyl)-6,7-dihydro-5H-thieno[3,2-b]pyran-6-yl]carbamate (7) (80 mg, 237.14 μmol, 76.67% yield) as a yellow solid. LC-MS: m/z [M+H-tBu]⁺=282.0

### Procedure for preparation of N-methyl-3-(trifluoromethyl)-6,7-dihydro-5H-thieno[3,2-b]pyran-6-amine hydrochloride (248)

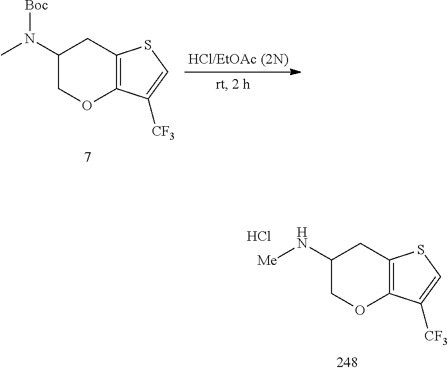 [SMILES: CN(C(=O)OC(C)(C)C)C1COc2c(C(F)(F)F)csc2C1 ; Cl ; CNC1COc2c(C(F)(F)F)csc2C1]

To a solution of tert-butyl N-methyl-N-[3-(trifluoromethyl)-6,7-dihydro-5H-thieno[3,2-b]pyran-6-yl]carbamate (7) (80 mg, 237.14 μmol, 1 eq) in EtOAc (1 mL) was added HCl/EtOAc (4 M, 1 mL, 16.87 eq). The mixture was stirred at 25° C. for 2 hr. The mixture was concentrated. The residue was purified by prep-HPLC (column: Phenomenex luna C18 150*25 mm*10 μm; mobile phase: [water(HCl)-ACN]; gradient:1%-25% B over 10 min) to afford N-methyl-3-(trifluoromethyl)-6,7-dihydro-5H-thieno[3,2-b]pyran-6-amine hydrochloride (248) (43.24 mg, 151.66 μmol, 63.96% yield) as a white solid. ¹H NMR (400 MHz, METHANOL-d4) δ=7.83 (s, 1H), 4.63-4.55 (m, 1H), 4.31 (d, J=12.8 Hz, 1H), 3.87 (br s, 1H), 3.40-3.33 (m, 1H), 3.12-3.03 (m, 1H), 2.83 (s, 3H). LC-MS: m/z [M+H]⁺=238.0

### Synthetic scheme for preparation of 3-cyclopropyl-N-methyl-4,5,6,7-tetrahydrobenzothiophen-5-amine hydrochloride enantiomer 1 (249) and enantiomer 2 (250)

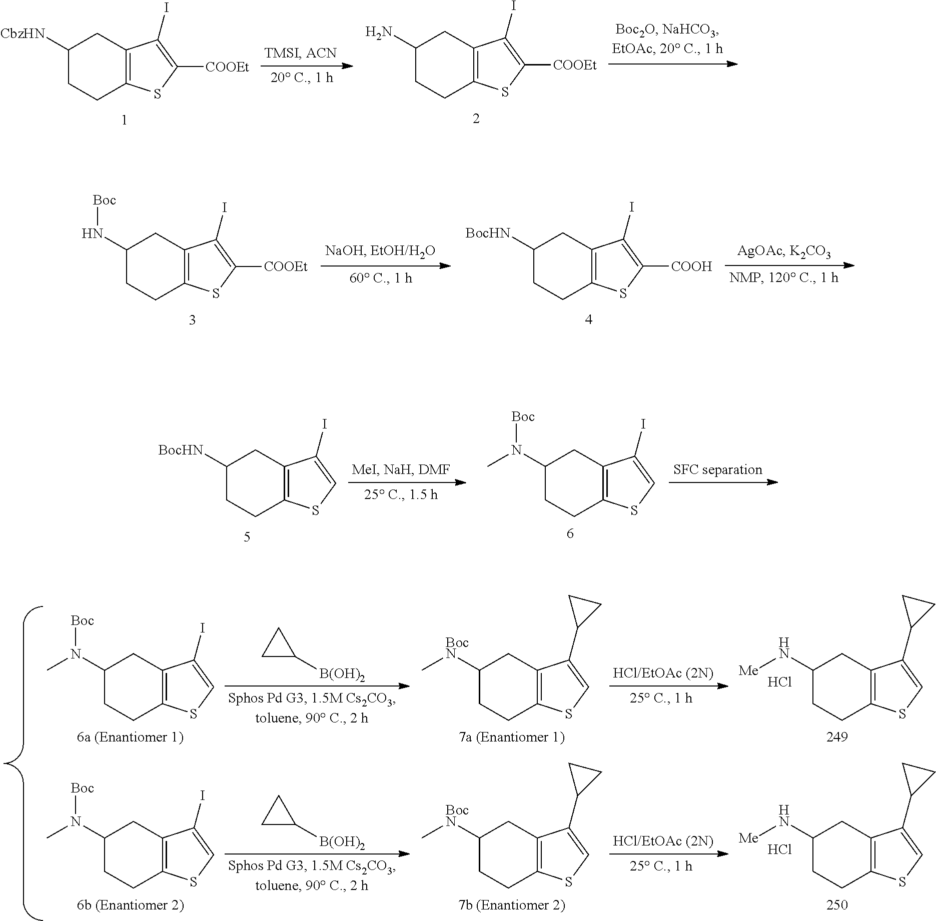

### Synthesis of ethyl 5-amino-3-iodo-4,5,6,7-tetrahydrobenzo[b]thiophene-2-carboxylate (2)

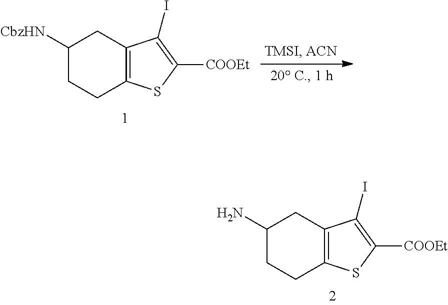 [SMILES: CCOC(=O)c1sc2c(c1I)CC(NC(=O)OCc1ccccc1)CC2 ; CCOC(=O)c1sc2c(c1I)CC(N)CC2]

To a solution of ethyl 5-(benzyloxycarbonylamino)-3-iodo-4,5,6,7-tetrahydrobenzothiophene-2-carboxylate (1) (8.4 g, 17.31 mmol, 1 eq) in ACN (80 mL) was added iodo(trimethyl)silane (10.39 g, 51.92 mmol, 7.07 mL, 3 eq). The mixture was stirred at 20° C. for 1 h. Then the reaction mixture was concentrated under reduced pressure to remove ACN. The crude compound ethyl 5-amino-3-iodo-4,5,6,7-tetrahydrobenzo[b]thiophene-2-carboxylate (2) was obtained as a yellow solid. LC-MS: m/z [M+H]⁺=352.0

### Preparation of ethyl 5-(tert-butoxycarbonylamino)-3-iodo-4,5,6,7-tetrahydrobenzothiophene-2-carboxylate (3)

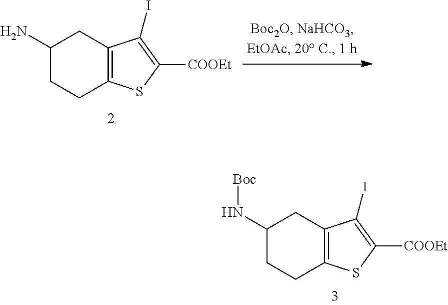 [SMILES: CCOC(=O)c1sc2c(c1I)CC(N)CC2 ; CCOC(=O)c1sc2c(c1I)CC(NC(=O)OC(C)(C)C)CC2]

To a solution of ethyl 5-amino-3-iodo-4,5,6,7-tetrahydrobenzo[b]thiophene-2-carboxylate (2) in EtOAC (80 mL) was added Boc₂O (4.53 g, 20.77 mmol, 4.77 mL, 1.2 eq) and aqueous NaHCO₃ (7.27 g, 86.54 mmol, 3.37 mL, 5 eq) at 20° C. and the mixture was stirred for 1 hr. The reaction mixture was quenched with H₂O (100 mL) at 20° C. and extracted with EtOAc (20 mL*2). The combined organic layers were washed with NaCl aq. saturated (25 mL*2), dried over Na₂SO₄, filtered and concentrated under reduced pressure to give a residue. The residue was purified by flash silica gel chromatography (ISCO®; 80 g SepaFlash® Silica Flash Column, eluent 0~25% Ethyl acetate/Petroleum ether gradient @60 mL/min). Compound ethyl 5-(tert-butoxycarbonylamino)-3-iodo-4,5,6,7-tetrahydrobenzothiophene-2-carboxylate (3) (6.2 g, 13.74 mmol, 79.37% yield) was obtained as a white solid. LC-MS: m/z [M+H-tBu]⁺=396.0

### Preparation of 5-(tert-butoxycarbonylamino)-3-iodo-4,5,6,7-tetrahydrobenzothiophene-2-carboxylic acid (4)

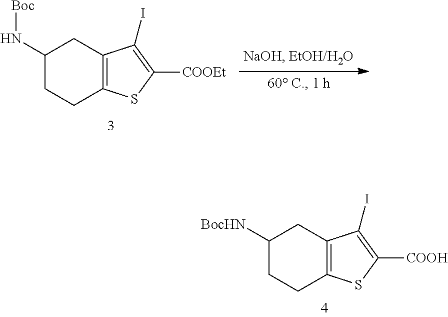 [SMILES: CCOC(=O)c1sc2c(c1I)CC(NC(=O)OC(C)(C)C)CC2 ; CC(C)(C)OC(=O)NC1CCc2sc(C(=O)O)c(I)c2C1]

To a solution of ethyl 5-(tert-butoxycarbonylamino)-3-iodo-4,5,6,7-tetrahydrobenzothiophene-2-carboxylate (3) (6.2 g, 13.74 mmol, 1 eq) in EtOH (60 mL) and H₂O (60 mL) was added NaOH (2.20 g, 54.95 mmol, 4 eq). The mixture was stirred at 60° C. for 1 hr. The reaction mixture was then cooled and concentrated under reduced pressure to remove EtOH. HCl (1M) was added and the pH was adjusted to 6,and then the suspension formed was filtered and the filter cake was collected. Compound 5-(tert-butoxycarbonylamino)-3-iodo-4,5,6,7-tetrahydrobenzothiophene-2-carboxylic acid (4) (5 g, 11.81 mmol, 85.99% yield) was obtained as a white solid. LC-MS: m/z [M+H-tBu]⁺=367.8 Preparation of tert-butyl N-(3-iodo-4,5,6,7-tetrahydrobenzothiophen-5-yl)carbamate (5)

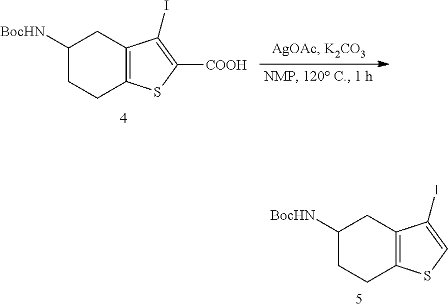 [SMILES: CC(C)(C)OC(=O)NC1CCc2sc(C(=O)O)c(I)c2C1 ; CC(C)(C)OC(=O)NC1CCc2scc(I)c2C1]

To a solution of 5-(tert-butoxycarbonylamino)-3-iodo-4,5,6,7-tetrahydrobenzothiophene-2-carboxylic acid (4) (5 g, 11.81 mmol, 1 eq) in NMP (40 mL) was added AgOAc (985.86 mg, 5.91 mmol, 0.5 eq) and K₂CO₃ (4.90 g, 35.44 mmol, 3 eq). The mixture was stirred at 120° C. for 1 hr. The mixture was then cooled and filtered and the filtrate was extracted with EtOAc (30 mL*3) and H₂O (40 ml). The combined organic layers were washed with aq. sat. NaCl (20 mL*3), dried over Na₂SO₄, filtered and concentrated under reduced pressure to give a residue. The residue was purified by flash silica gel chromatography (ISCO®; 80 g SepaFlash® Silica Flash Column, eluent 0~20% Ethyl acetate/Petroleum ether gradient @60 mL/min).Compound tert-butyl N-(3-iodo-4,5,6,7-tetrahydrobenzothiophen-5-yl)carbamate (5)(4 g, 10.55 mmol, 89.28% yield) was obtained as a white solid. LC-MS: m/z [M+H-tBu]⁺=324.0

### Preparation of tert-butyl N-(3-iodo-4,5,6,7-tetrahydrobenzothiophen-5-yl)-N-methyl-carbamate (6)

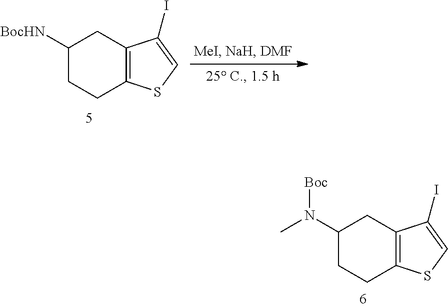 [SMILES: CC(C)(C)OC(=O)NC1CCc2scc(I)c2C1 ; CN(C(=O)OC(C)(C)C)C1CCc2scc(I)c2C1]

To a solution of tert-butyl N-(3-iodo-4,5,6,7-tetrahydrobenzothiophen-5-yl)carbamate (5) (4 g, 10.55 mmol, 1 eq) in DMF (40 mL) was added NaH (506.21 mg, 12.66 mmol, 60% suspension in mineral oil, 1.2 eq). The mixture was stirred at 0° C. for 0.5 h, then MeI (2.25 g, 15.82 mmol, 984.88 μL, 1.5 eq) was added and the mixture was stirred and allowed to naturally warm to 25° C. After stirring for 1 h the reaction mixture was quenched by addition aq. sat. NH₄Cl (100 mL) at 0° C. and extracted with EtOAc (50 mL*2). The combined organic layers were washed with aq. NaCl 100 mL (50 mL*2), dried over Na₂SO₄, filtered and concentrated under reduced pressure to give a residue. The residue was purified by flash silica gel chromatography (ISCO®; 80 g SepaFlash® Silica Flash Column, eluent 0~20% Ethyl acetate/Petroleum ether gradient @60 mL/min). Compound tert-butyl N-(3-iodo-4,5,6,7-tetrahydrobenzothiophen-5-yl)-N-methyl-carbamate (6) (2.5 g, 6.36 mmol, 60.27% yield) was obtained as a white solid. LC-MS: m/z [M+H]⁺=338.0 Preparation of tert-butyl N-(3-iodo-4,5,6,7-tetrahydrobenzothiophen-5-yl)-N-methyl-carbamate enantiomer 1 (6a) and enantiomer 2 (6b)

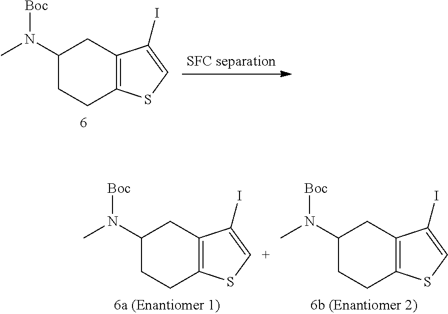 [SMILES: CN(C(=O)OC(C)(C)C)C1CCc2scc(I)c2C1 ; CN(C(=O)OC(C)(C)C)C1CCC2=C(C1)C(I)=[C+]S2 ; CN(C(=O)OC(C)(C)C)C1CCc2scc(I)c2C1]

Racemic tert-butyl N-(3-iodo-4,5,6,7-tetrahydrobenzothiophen-5-yl)-N-methyl-carbamate (6) (2.5 g) was separated to its enantiomers by SFC (condition:column: DAICEL CHIRALPAK IG (250 mm*50 mm, 10 μm); mobile phase: [CO₂-i-PrOH (0.1% NH₃H₂O)]; B %:12%, isocratic elution mode). Compound tert-butyl N-(3-iodo-4,5,6,7-tetrahydrobenzothiophen-5-yl)-N-methyl-carbamate enantiomer 1 (6a) (1 g, 2.54 mmol, 40.00% yield) was obtained as a white solid. Compound tert-butyl N-(3-iodo-4,5,6,7-tetrahydrobenzothiophen-5-yl)-N-methyl-carbamate enantiomer 2 (6b) (1 g, 2.54 mmol, 40.00% yield) was obtained as a white solid.

### Preparation of tert-butyl N-(3-cyclopropyl-4,5,6,7-tetrahydrobenzothiophen-5-yl)-N-methyl-carbamate enantiomer 1 (7a)

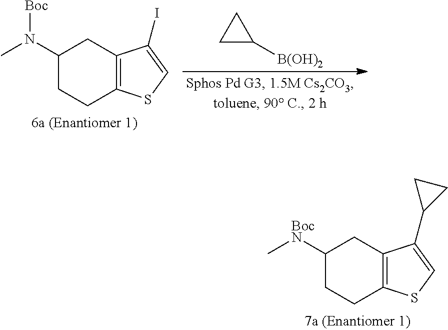 [SMILES: CN(C(=O)OC(C)(C)C)C1CCc2scc(I)c2C1 ; OB(O)C1CC1 ; CN(C(=O)OC(C)(C)C)C1CCc2scc(C3CC3)c2C1]

To a solution of tert-butyl N-(3-iodo-4,5,6,7-tetrahydrobenzothiophen-5-yl)-N-methyl-carbamate enantiomer 1 (6a) (150 mg, 381.41 μmol, 1 eq) in Tol. (1 mL) was added Sphos Pd G3 (29.76 mg, 38.14 μmol, 0.1 eq), cyclopropylboronic acid (163.81 mg, 1.91 mmol, 5 eq), and aq. cesium carbonate (1.5 M, 762.81 μL, 3 eq) and the mixture formed was purged with N₂ for 3 times. The mixture was stirred at 90° C. for 2 hr under N₂ atmosphere then cooled and concentrated under reduced pressure to remove toluene. The residue was purified by column chromatography (SiO2, Petroleum ether/Ethyl acetate=5/1). Compound tert-butyl N-(3-cyclopropyl-4,5,6,7-tetrahydrobenzothiophen-5-yl)-N-methyl-carbamate enantiomer 1 (7a) (46 mg, 149.62 μmol, 39.23% yield) was obtained as a white solid. LC-MS: m/z [M+H]⁺=252.0 Preparation of tert-butyl N-(3-cyclopropyl-4,5,6,7-tetrahydrobenzothiophen-5-yl)-N-methyl-carbamate enantiomer 2 (7b)

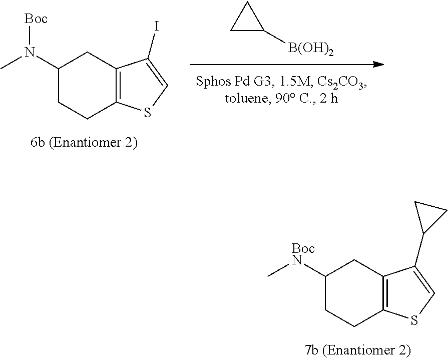 [SMILES: CN(C(=O)OC(C)(C)C)C1CCc2scc(I)c2C1 ; OB(O)C1CC1 ; CN(C(=O)OC(C)(C)C)C1CCc2scc(C3CC3)c2C1]

To a solution of tert-butyl N-(3-iodo-4,5,6,7-tetrahydrobenzothiophen-5-yl)-N-methyl-carbamate enantiomer 2 (6b) (150 mg, 381.41 μmol, 1 eq) in Tol. (1 mL) was added Sphos Pd G3 (29.76 mg, 38.14 μmol, 0.1 eq), cyclopropylboronic acid (163.81 mg, 1.91 mmol, 5 eq) and aq. cesium carbonate (1.5 M, 762.81 μL, 3 eq). The mixture was stirred at 90° C. for 2 hr under N₂ atmosphere. The reaction mixture was then cooled and concentrated under reduced pressure to remove toluene. The residue was purified by column chromatography (SiO2, Petroleum ether/Ethyl acetate=5/1). Compound tert-butyl N-(3-cyclopropyl-4,5,6,7-tetrahydrobenzothiophen-5-yl)-N-methyl-carbamate enantiomer 2 (7b) (50 mg, 162.63 μmol, 42.64% yield) was obtained as a white solid. LC-MS: m/z [M+H]⁺=252.0

### Preparation of 3-cyclopropyl-N-methyl-4,5,6,7-tetrahydrobenzothiophen-5-amine hydrochloride enantiomer 1 (249)

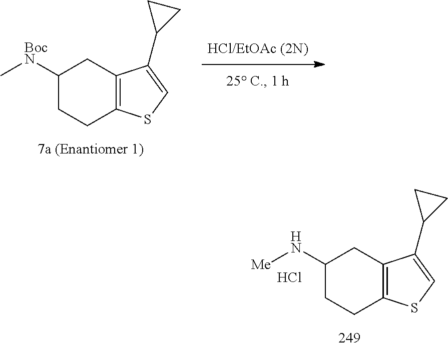 [SMILES: CN(C(=O)OC(C)(C)C)C1CCc2scc(C3CC3)c2C1 ; CNC1CCc2scc(C3CC3)c2C1 ; Cl]

To a solution of tert-butyl N-(3-cyclopropyl-4,5,6,7-tetrahydrobenzothiophen-5-yl)-N-methyl-carbamate enantiomer 1 (7a) (46 mg, 149.62 μmol, 1 eq) in EtOAc (2 mL) was added HCl/EtOAc (4 M, 2 mL, 53.47 eq). The mixture was stirred at 25° C. for 1 hr. The reaction mixture was then concentrated under reduced pressure to remove EtOAc. The residue was suspended in EtOAc at 25° C. for 10 min then filtered collected and dried. Compound 3-cyclopropyl-N-methyl-4,5,6,7-tetrahydrobenzothiophen-5-amine hydrochloride enantiomer 1 (249) (36.18 mg, 145.66 μmol, 97.35% yield) was obtained as a white solid. LC-MS: m/z [M+H]⁺=208.0. ¹H NMR (400 MHz, METHANOL-d4) δ=6.72 (s, 1H), 3.56 (dddd, J=2.9, 5.7, 8.4, 11.0 Hz, 1H), 3.30-3.18 (m, 1H), 3.01-2.91 (m, 2H), 2.84 (s, 3H), 2.70 (br dd, J=9.0, 15.8 Hz, 1H), 2.35 (br dd, J=3.4, 9.2 Hz, 1H), 2.06-1.91 (m, 1H), 1.79-1.66 (m, 1H), 0.93-0.83 (m, 2H), 0.65-0.51 (m, 2H)

### Preparation of 3-cyclopropyl-N-methyl-4,5,6,7-tetrahydrobenzothiophen-5-amine hydrochloride enantiomer 2 (250)

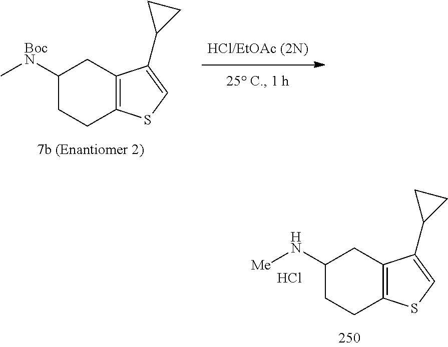 [SMILES: CN(C(=O)OC(C)(C)C)C1CCc2scc(C3CC3)c2C1 ; CNC1CCc2scc(C3CC3)c2C1 ; Cl]

To a solution of tert-butyl N-(3-cyclopropyl-4,5,6,7-tetrahydrobenzothiophen-5-yl)-N-methyl-carbamate enantiomer 2 (7b) (50 mg, 162.63 μmol, 1 eq) in EtOAc (2 mL) was added HCl/EtOAc (4 M, 2 mL, 49.19 eq). The mixture was stirred at 25° C. for 1 hr. The reaction mixture was then concentrated under reduced pressure to remove EtOAc. with the residue was suspended in EtOAc at 25° C. for 10 min. The mixture was then filtered, collected and dried. Compound 3-cyclopropyl-N-methyl-4,5,6,7-tetrahydrobenzothiophen-5-amine enantiomer 2 (250) (36.97 mg, 150.49 μmol, 92.54% yield) was obtained as a white solid. LC-MS: m/z [M+H]⁺=208.0. ¹H NMR (400 MHz, METHANOL-d4) δ=6.72 (s, 1H), 3.61-3.51 (m, 1H), 3.24 (br dd, J=5.3, 15.6 Hz, 1H), 3.01-2.91 (m, 2H), 2.84 (s, 3H), 2.70 (br dd, J=8.9, 15.7 Hz, 1H), 2.38-2.31 (m, 1H), 2.07-1.92 (m, 1H), 1.77-1.67 (m, 1H), 0.94-0.82 (m, 2H), 0.62-0.54 (m, 2H)

### Synthetic scheme for preparation of 3-ethyl-N-methyl-4,5,6,7-tetrahydrobenzothiophen-5-amine hydrochloride enantiomer 1 (251) and enantiomer 2 (252)

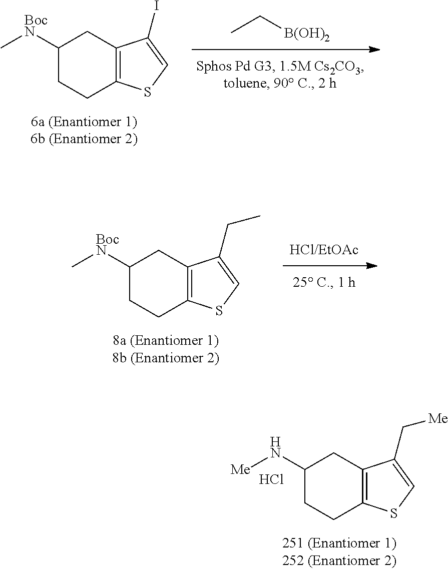

### Preparation of tert-butyl N-(3-ethyl-4,5,6,7-tetrahydrobenzothiophen-5-yl)-N-methyl-carbamate (8a)

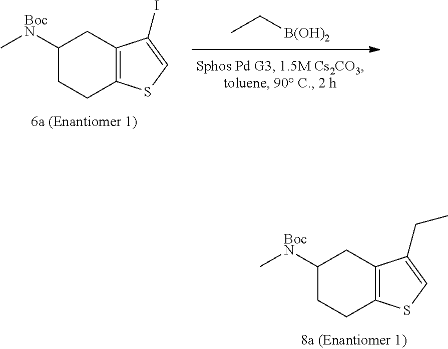

To a solution of tert-butyl N-(3-iodo-4,5,6,7-tetrahydrobenzothiophen-5-yl)-N-methyl-carbamate enantiomer 1 (6a) (150 mg, 381.41 μmol, 1 eq) in Tol. (1 mL) was added Sphos Pd G3 (29.76 mg, 38.14 μmol, 0.1 eq), ethylboronic acid (140.90 mg, 1.91 mmol, 5 eq) and aq. cesium carbonate (1.5 M, 762.81 μL, 3 eq) and the mixture purged with N₂ for 3 times. The mixture was stirred at 90° C. for 2 hr under N₂ atmosphere then cooled and concentrated under reduced pressure to remove toluene. The residue was purified by column chromatography (SiO₂, Petroleum ether/Ethyl acetate=5/1). Compound tert-butyl N-(3-ethyl-4,5,6,7-tetrahydrobenzothiophen-5-yl)-N-methyl-carbamate enantiomer 1 (8a) (50 mg, 169.24 μmol, 44.37% yield) was obtained as a white solid. LC-MS: m/z [M+H-tBu]⁺=240.0

### Preparation of tert-butyl N-(3-ethyl-4,5,6,7-tetrahydrobenzothiophen-5-yl)-N-methyl-carbamate enantiomer 2 (8b)

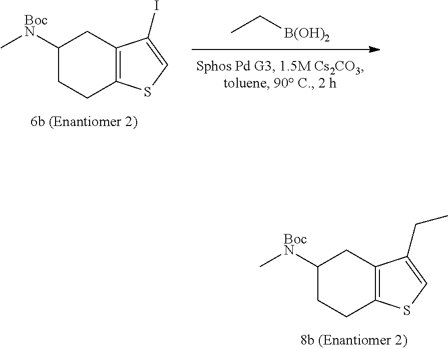

To a solution of tert-butyl N-(3-iodo-4,5,6,7-tetrahydrobenzothiophen-5-yl)-N-methyl-carbamate (150 mg, 381.41 μmol, 1 eq) in Tol. (1 mL) was added Sphos Pd G3 (29.76 mg, 38.14 μmol, 0.1 eq), ethylboronic acid (140.90 mg, 1.91 mmol, 5 eq), cesium carbonate aq. (1.5 M, 762.81 μL, 3 eq) and then the mixture was purged with N₂ for 3 times. The mixture was stirred at 90° C. for 2 hr under N₂ atmosphere. The reaction mixture was then cooled and concentrated under reduced pressure to remove toluene. The residue was purified by column chromatography (SiO2, Petroleum ether/Ethyl acetate=5/1). Compound tert-butyl N-(3-ethyl-4,5,6,7-tetrahydrobenzothiophen-5-yl)-N-methyl-carbamate enantiomer 2 (8b) (70 mg, 236.94 μmol, 62.12% yield) was obtained as a white solid. LC-MS: m/z [M+H-tBu]⁺=240.0

### Preparation of 3-ethyl-N-methyl-4,5,6,7-tetrahydrobenzothiophen-5-amine hydrochloride enantiomer 1 (251)

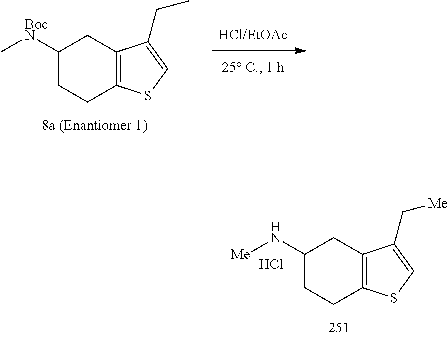 [SMILES: CCc1csc2c1CC(N(C)C(=O)OC(C)(C)C)CC2 ; CCc1csc2c1CC(NC)CC2 ; Cl]

To a solution of tert-butyl N-(3-ethyl-4,5,6,7-tetrahydrobenzothiophen-5-yl)-N-methyl-carbamate enantiomer 1 (8a) (50 mg, 169.24 μmol, 1 eq) in EtOAc (1 mL) was added HCl/EtOAc (4 M, 2 mL, 49.19 eq). The mixture was stirred at 25° C. for 1 hr and then concentrated under reduced pressure to remove EtOAc. The residue was suspended in EtOAc at 25° C. for 10 min and then the suspension was filtered, collected and dried. Compound 3-ethyl-N-methyl-4,5,6,7-tetrahydrobenzothiophen-5-amine hydrochloride enantiomer 1 (251) (36.73 mg, 154.68 μmol, 91.40% yield) was obtained as a white solid. LC-MS: m/z [M+H]⁺=196.0. ¹H NMR (400 MHz, METHANOL-d₄) δ=6.88 (s, 1H), 3.58-3.49 (m, 1H), 3.12-3.04 (m, 1H), 3.00-2.93 (m, 2H), 2.83 (s, 3H), 2.63-2.46 (m, 3H), 2.38-2.29 (m, 1H), 2.03-1.91 (m, 1H), 1.25 (t, J=7.4 Hz, 3H)

### Preparation of 3-ethyl-N-methyl-4,5,6,7-tetrahydrobenzothiophen-5-amine hydrochloride enantiomer 2 (252)

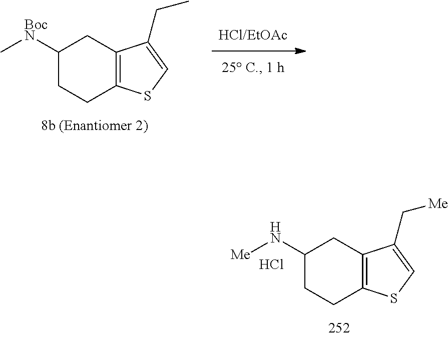 [SMILES: CCc1csc2c1CC(N(C)C(=O)OC(C)(C)C)CC2 ; CCc1csc2c1CC(NC)CC2 ; Cl]

To a solution of tert-butyl N-(3-ethyl-4,5,6,7-tetrahydrobenzothiophen-5-yl)-N-methyl-carbamate enantiomer 2 (8b) (70 mg, 236.94 μmol, 1 eq) in EtOAc (1 mL) was added HCl/EtOAc (4 M, 2 mL, 33.76 eq). The mixture was stirred at 25° C. for 1 hr. The reaction mixture was then concentrated under reduced pressure to remove EtOAC. The residue was purified by prep-HPLC (HCl condition:column: Phenomenex luna C18 150*25 mm*10 μm; mobile phase: [water(HCl)-ACN]; gradient:5%-35% B over 10 min).Compound 3-ethyl-N-methyl-4,5,6,7-tetrahydrobenzothiophen-5-amine hydrochloride enantiomer 2 (252) (17.79 mg, 75.70 μmol, 31.95% yield) was obtained as a white solid. LC-MS: m/z [M+H⁺=196.0. ¹H NMR (400 MHz, METHANOL-d₄) δ=6.88 (s, 1H), 3.59-3.49 (m, 1H), 3.12-3.04 (m, 1H), 3.01-2.92 (m, 2H), 2.83 (s, 3H), 2.64-2.46 (m, 3H), 2.38-2.29 (m, 1H), 2.03-1.92 (m, 1H), 1.25 (t, J=7.5 Hz, 3H)

### Synthetic scheme for preparation of 3-(trifluoromethyl)-4,5,6,7-tetrahydrobenzothiophen-5-amine hydrochloride enantiomer 1 (254) and enantiomer 2 (255)

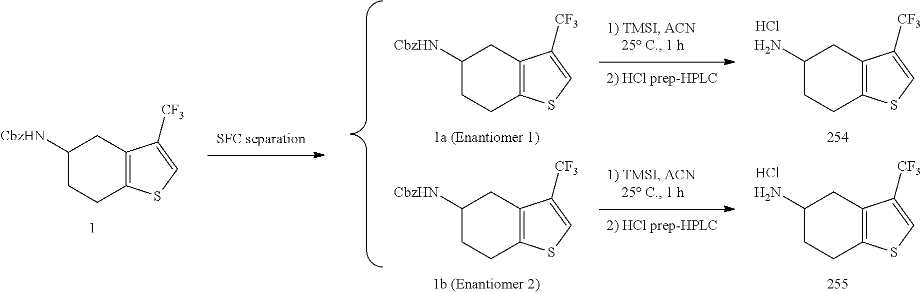

### Procedure for preparation of benzyl N-[3-(trifluoromethyl)-4,5,6,7-tetrahydrobenzothiophen-5-yl]carbamate enantiomer 1 (1a) and enantiomer 2 (1b)

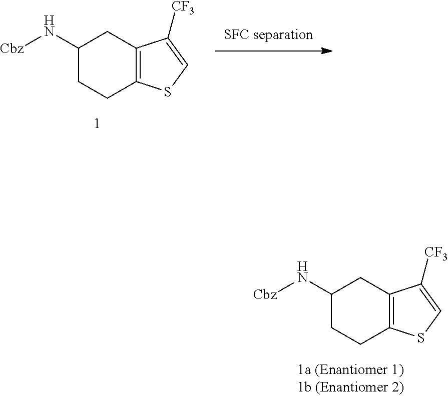 [SMILES: O=C(NC1CCc2scc(C(F)(F)F)c2C1)OCc1ccccc1 ; O=C(NC1CCc2scc(C(F)(F)F)c2C1)OCc1ccccc1]

Racemic benzyl N-[3-(trifluoromethyl)-4,5,6,7-tetrahydrobenzothiophen-5-yl]carbamate (300 mg) was separated to its enantiomers by SFC (column: DAICEL CHIRALCEL OX (250 mm*50 mm, 10 μm); mobile phase: [CO₂-MeOH (0.1% NH₃H₂O)]; B %:10%, isocratic elution mode). Compound benzyl N-[3-(trifluoromethyl)-4,5,6,7-tetrahydrobenzothiophen-5-yl]carbamate enantiomer 1 (1a) (130 mg, 365.81 mol) was obtained as a white solid. Compound benzyl N-[3-(trifluoromethyl)-4,5,6,7-tetrahydrobenzothiophen-5-yl]carbamate enantiomer 2 (1b) (120 mg, 337.67 mol) was obtained as a white solid.

### Procedure for preparation of 3-(trifluoromethyl)-4,5,6,7-tetrahydrobenzothiophen-5-amine hydrochloride enantiomer 1 (254)

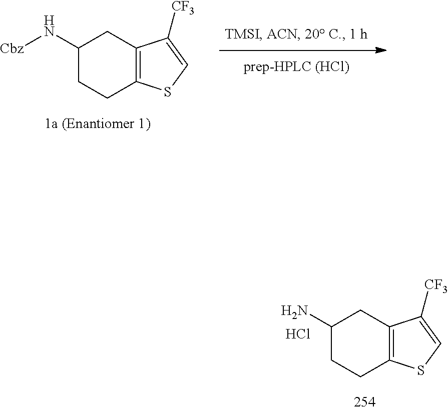 [SMILES: O=C(NC1CCc2scc(C(F)(F)F)c2C1)OCc1ccccc1 ; Cl ; NC1CCc2scc(C(F)(F)F)c2C1]

To a solution of benzyl N-[3-(trifluoromethyl)-4,5,6,7-tetrahydrobenzothiophen-5-yl]carbamate enantiomer 1 (1a) (130 mg, 365.81 μmol, 1 eq) in ACN (2 mL) was added TMSI (146.39 mg, 731.62 μmol, 99.59 μL, 2 eq). The mixture was stirred at 25° C. for 1 hr. Solvent was removed and the residue was purified by prep-HPLC (column: Phenomenex luna C18 150*25 mm*10 μm; mobile phase: [water(HCl)-ACN]; gradient:1%-30% B over 10 min). Compound 3-(trifluoromethyl)-4,5,6,7-tetrahydrobenzothiophen-5-amine hydrochloride enantiomer 1 (254) (50.66 mg, 193.83 μmol, 52.99% yield) was obtained as a white solid. ¹H NMR (400 MHz, METHANOL-d4) δ=7.88-7.82 (m, 1H), 3.71-3.59 (m, 1H), 3.24-3.15 (m, 1H), 3.10-2.93 (m, 2H), 2.80-2.67 (m, 1H), 2.37-2.20 (m, 1H), 2.08-1.88 (m, 1H). LCMS: m/z [M+H]⁺=222.1

### Procedure for preparation of 3-(trifluoromethyl)-4,5,6,7-tetrahydrobenzothiophen-5-amine hydrochloride enantiomer 2 (255)

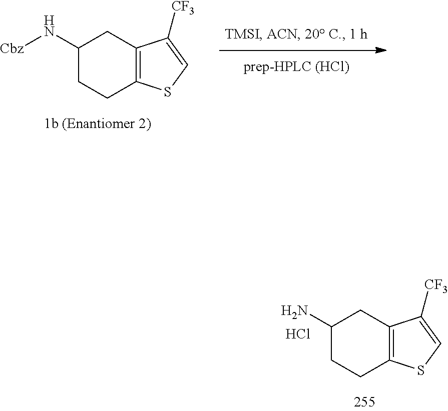 [SMILES: O=C(NC1CCc2scc(C(F)(F)F)c2C1)OCc1ccccc1 ; Cl ; NC1CCc2scc(C(F)(F)F)c2C1]

To a solution of benzyl N-[3-(trifluoromethyl)-4,5,6,7-tetrahydrobenzothiophen-5-yl]carbamate enantiomer 2 (1b) (120 mg, 337.67 μmol, 1 eq) in ACN (2 mL) was added TMSI (135.13 mg, 675.34 μmol, 91.93 μL, 2 eq). The mixture was stirred at 25° C. for 1 hr. Remove the solvent on a rotary evaporator. The residue was purified by prep-HPLC (column: Phenomenex luna C18 150*25 mm*10 μm; mobile phase: [water(HCl)-ACN]; gradient:1%-30% B over 10 min). Compound 3-(trifluoromethyl)-4,5,6,7-tetrahydrobenzothiophen-5-amine (58.31 mg, 221.06 μmol, 65.47% yield) was obtained as a white solid. ¹H NMR (400 MHz, METHANOL-d4) δ=7.84 (s, 1H), 3.74-3.58 (m, 1H), 3.20 (br dd, J=5.1, 16.2 Hz, 1H), 3.09-2.91 (m, 2H), 2.80-2.66 (m, 1H), 2.37-2.21 (m, 1H), 2.07-1.90 (m, 1H). LCMS: m/z [M+H]⁺=222.1

### Synthetic scheme for preparation of N-methyl-2-phenyl-4,5,6,7-tetrahydrobenzothiophen-5-amine hydrochloride enantiomer 1 (256) and enantiomer2 (257)

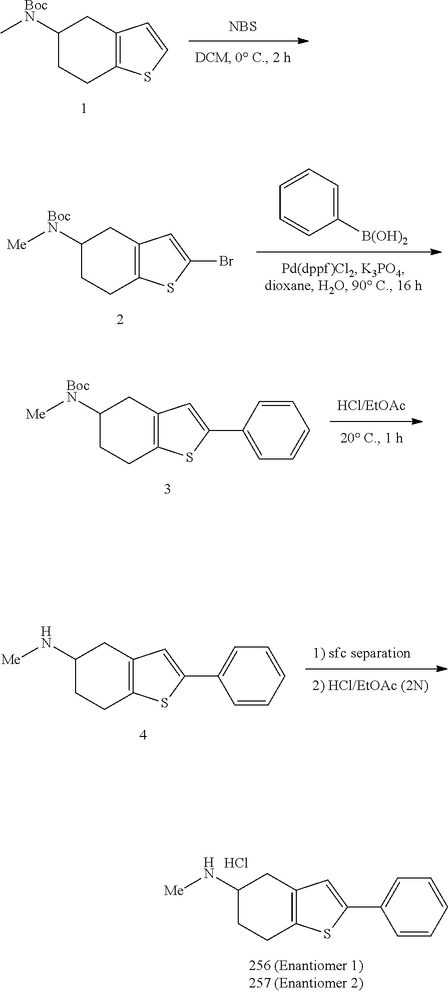

### Procedure for preparation of tert-butyl N-(2-bromo-4,5,6,7-tetrahydrobenzothiophen-5-yl)-N-methyl-carbamate (2)

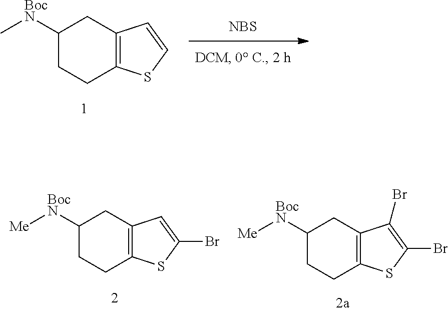 [SMILES: CN(C(=O)OC(C)(C)C)C1CCc2sccc2C1 ; "NBS" ; CN(C(=O)OC(C)(C)C)C1CCc2sc(Br)cc2C1 ; CN(C(=O)OC(C)(C)C)C1CCc2sc(Br)c(Br)c2C1]

To a solution of tert-butyl N-methyl-N-(4,5,6,7-tetrahydrobenzothiophen-5-yl)carbamate (1) (800 mg, 2.99 mmol, 1 eq) in DCM (10 mL) was added NBS (639.02 mg, 3.59 mmol, 1.2 eq). The mixture was stirred at 0° C. for 2 hr. The reaction mixture was diluted with H₂O (10 mL) and extracted with EtOAc (30 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~12% Ethyl acetate/Petroleum ether gradient @40 mL/min). Compound tert-butyl N-(2-bromo-4,5,6,7-tetrahydrobenzothiophen-5-yl)-N-methyl-carbamate (2) (900 mg) was obtained as a colorless oil that contained ~15% of dibromo derivative tert-butyl (2,3-dibromo-4,5,6,7-tetrahydrobenzo[b]thiophen-5-yl)(methyl)carbamate(2a). LCMS: m/z [M+H-Bu]⁺=290.0

### Procedure for preparation of tert-butyl N-methyl-N-(2-phenyl-4,5,6,7-tetrahydrobenzothiophen-5-yl)carbamate (3)

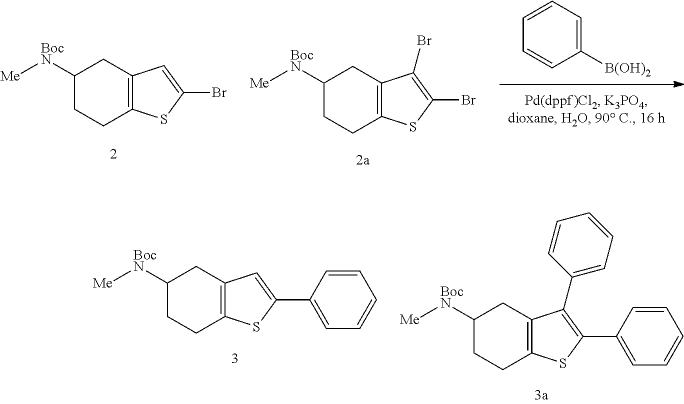

A mixture of tert-butyl N-(2-bromo-4,5,6,7-tetrahydrobenzothiophen-5-yl)-N-methyl-carbamate (2) and tert-butyl (2,3-dibromo-4,5,6,7-tetrahydrobenzo[b]thiophen-5-yl)(methyl)carbamate (2a) (900 mg), phenylboronic acid (1.58 g, 13.00 mmol, 5 eq), Pd(dppf)Cl₂ (95.09 mg, 129.95 μmol, 0.05 eq) and K₃PO₄ (1.66 g, 7.80 mmol, 3 eq) in dioxane (15 mL) and H₂O (1 mL) was degassed and purged with N₂ 3 times, and then the mixture was stirred at 90° C. for 16 hr under N₂ atmosphere. The reaction mixture was then cooled and diluted with H₂O (10 mL) and extracted with EtOAc (10 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 4 g SepaFlash® Silica Flash Column, eluent 0-15% Ethyl acetate/Petroleum ether gradient @40 mL/min). The residue was purified by prep-HPLC (column: Welch Ultimate XB-CN 250*50*10 μm; mobile phase: [Hexane-EtOH]; B %:1%, isocratic elution mode). Compound tert-butyl N-methyl-N-(2-phenyl-4,5,6,7-tetrahydrobenzothiophen-5-yl)carbamate (3) (620 mg) was obtained as a white solid that contained ~15% of tert-butyl (2,3-diphenyl-4,5,6,7-tetrahydrobenzo[b]thiophen-5-yl)(methyl)carbamate (3a). LCMS: m/z [M+H-Bu]⁺=288.1

### Procedure for preparation of N-methyl-2-phenyl-4,5,6,7-tetrahydrobenzothiophen-5-amine (4)

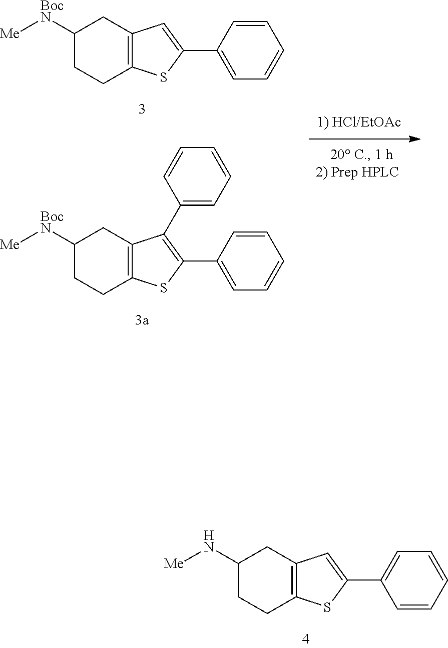 [SMILES: CN(C(=O)OC(C)(C)C)C1CCc2sc(-c3ccccc3)cc2C1 ; CN(C(=O)OC(C)(C)C)C1CCc2sc(-c3ccccc3)c(-c3ccccc3)c2C1 ; CNC1CCc2sc(-c3ccccc3)cc2C1]

To a solution of tert-butyl N-methyl-N-(2-phenyl-4,5,6,7-tetrahydrobenzothiophen-5-yl)carbamate (3) and tert-butyl (2,3-diphenyl-4,5,6,7-tetrahydrobenzo[b]thiophen-5-yl)(methyl)carbamate (3a) (620 mg) in EtOAc (5 mL) was added HCl/EtOAc (4 M, 5 mL). The mixture was stirred at 20° C. for 1 hr. The solvent was then removed and the residue was purified by prep-HPLC (column: Phenomenex luna C18 150*25 mm*10 μm; mobile phase: [water(HCl)-ACN]; gradient:13%-43% B over 10 min). To the compound obtained from this purification was added MeOH (2 mL). Followed addition of 2 g of anion exchange resin (AMBERLITE(R)IRA-400 (CL, cas: 122560-63-8). The mixture was stirred and then filtered and the filtrate was concentrated to give the product. N-methyl-2-phenyl-4,5,6,7-tetrahydrobenzothiophen-5-amine (4) (270 mg, 1.11 mmol, 61.46% yield) as a white solid. LCMS: m/z [M+H]⁺=244.2

### Procedure for preparation of N-methyl-2-phenyl-4,5,6,7-tetrahydrobenzothiophen-5-amine hydrochloride enantiomer 1 (256) and enantiomer 2 (257)

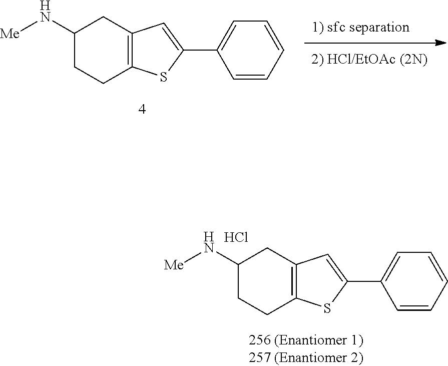 [SMILES: CNC1CCc2sc(-c3ccccc3)cc2C1 ; Cl ; CNC1CCc2sc(-c3ccccc3)cc2C1]

Racemic N-methyl-2-phenyl-4,5,6,7-tetrahydrobenzothiophen-5-amine (4) was separated to its enatiomers by SFC (column: DAICEL CHIRALCEL OD (250 mm*30 mm, 10 μm); mobile phase: [CO₂-MeOH (0.1% NH₃H₂O)]; B %:30%, isocratic elution mode). Each separated enantiomer was dissolved in EtOAc (5 ml). Each solution was treated with HCl/EtOAc (5 ml) to form a mixture of the HCl salt. Each mixture was filtered and the filter cake was washed with EtOAc (10 mL) to afford the HCl salt of each enantiomer. N-methyl-2-phenyl-4,5,6,7-tetrahydrobenzothiophen-5-amine hydrochloride enantiomer 1 (256) (123.37 mg, 440.88 μmol, 39.74% yield) was isolated as an off-white solid. ¹H NMR (400 MHz, METHANOL-d4) δ=7.56 (d, J=7.4 Hz, 2H), 7.35 (t, J=7.7 Hz, 2H), 7.29-7.23 (m, 1H), 7.11 (s, 1H), 3.60-3.50 (m, 1H), 3.19 (br dd, J=4.8, 15.8 Hz, 1H), 3.05-2.91 (m, 2H), 2.81 (s, 3H), 2.73 (br dd, J=9.0, 15.8 Hz, 1H), 2.42-2.29 (m, 1H), 2.06-1.93 (m, 1H). LCMS: m/z [M+H]⁺=244.2. Compound N-methyl-2-phenyl-4,5,6,7-tetrahydrobenzothiophen-5-amine hydrochloride enantiomer 2 (257) (131.16 mg, 468.72 μmol, 42.25% yield) was obtained as an off-white solid. ¹H NMR (400 MHz, METHANOL-d4) δ=7.58 (d, J=8.0 Hz, 2H), 7.37 (t, J=7.7 Hz, 2H), 7.31-7.24 (m, 1H), 7.13 (s, 1H), 3.62-3.51 (m, 1H), 3.21 (br dd, J=4.9, 15.8 Hz, 1H), 3.07-2.94 (m, 2H), 2.83 (s, 3H), 2.75 (br dd, J=9.1, 15.8 Hz, 1H), 2.45-2.33 (m, 1H), 2.09-1.94 (m, 1H). LCMS: m/z [M+H]⁺=244.2

### Synthetic scheme for preparation of N-methyl-5-(trifluoromethyl)-3,4-dihydro-2H-thieno[3,4-b]pyran-3-amine hydrochloride enantiomer 1 (258), and enantiomer 2 (259)

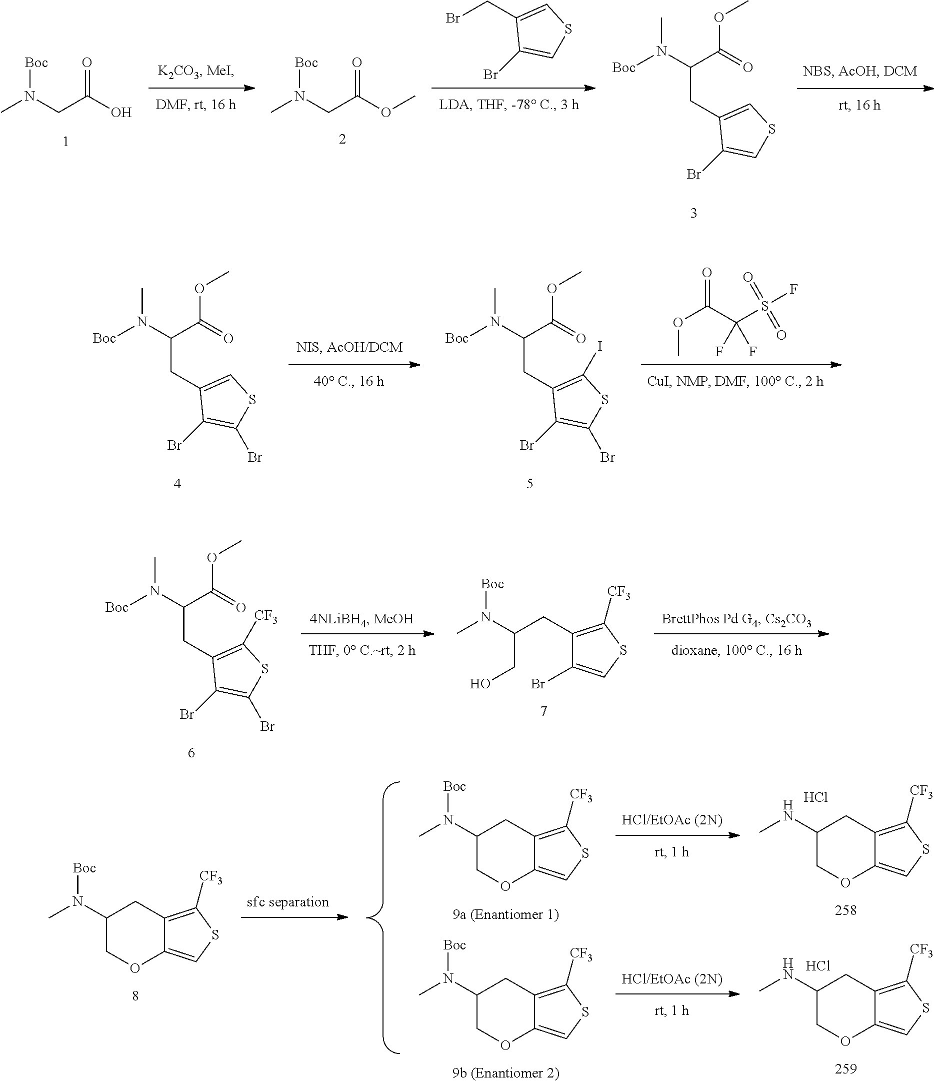

### Procedure for preparation of methyl 2-[tert-butoxycarbonyl(methyl)amino]acetate 2

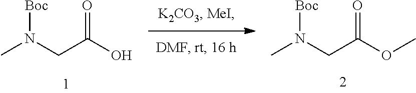

To a solution of 2-[tert-butoxycarbonyl(methyl)amino]acetic acid (1) (50 g, 264.26 mmol, 1 eq) in DMF (300 mL) was added K₂CO₃ (44 g, 317.11 mmol, 1.2 eq) and iodomethane (41.3 g, 290.68 mmol, 18.10 mL, 1.1 eq). The mixture was stirred at room temperature for 16 hrs. The reaction mixture was then filtered and the filtrate was diluted with H₂O 1 L and extracted with EtOAc (200 mL*3). The combined organic layers were washed with brine (500 mL), dried over anhydrous Na₂SO₄, filtered and concentrated under reduced pressure to give a residue. The residue was purified by column chromatography (SiO2, Petroleum ether/Ethyl acetate=1/0 to 5/1) to give methyl 2-[tert-butoxycarbonyl(methyl)amino]acetate (2) (52 g, 255.86 mmol, 96.82% yield) as a yellow oil. ¹H NMR (400 MHz, CHLOROFORM-d) δ=4.02-3.86 (m, 2H), 3.74 (d, J=2.6 Hz, 3H), 2.93 (d, J=6.4 Hz, 3H), 1.45 (d, J=18.5 Hz, 9H)

### Procedure for preparation of methyl 3-(4-bromo-3-thienyl)-2-[tert-butoxycarbonyl(methyl)amino]propanoate (3)

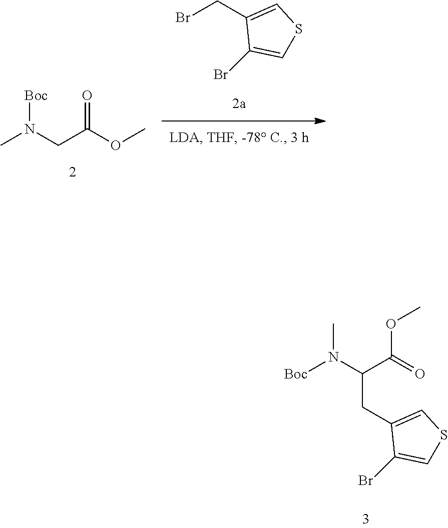

To a solution of LDA (2 M, 54.2 mL, 1.1 eq) in THF (150 mL) was added a solution of methyl 2-[tert-butoxycarbonyl(methyl)amino]acetate (2) (20 g, 98.41 mmol, 1 eq) in THF (30 mL) at −78° C. dropwise, after 1 h a solution of 3-bromo-4-(bromomethyl)thiophene (26.5 g, 103.33 mmol, 1.05 eq) in THF (20 mL) was added dropwise. The mixture was stirred at −78° C. for 2 h under N₂. The reaction mixture was then quenched with aq. sat.NH₄Cl (300 mL) at 0° C., and then extracted with EtOAc (200 mL*3). The combined organic layers were washed with brine (200 mL), dried over anhydrous Na₂SO₄, filtered and concentrated under reduced pressure to give a residue. The residue was purified by flash silica gel chromatography (ISCO®; 120 g SepaFlash® Silica Flash Column, eluent 0~15% Ethyl acetate/Petroleum ether gradient @70 mL/min) to give methyl 3-(4-bromo-3-thienyl)-2-[tert-butoxycarbonyl(methyl)amino]propanoate (3) (20 g, 52.87 mmol, 53.73% yield) as a yellow oil. ¹H NMR (400 MHz, CHLOROFORM-d) δ=7.27-7.23 (m, 1H), 7.11-7.00 (m, 1H), 4.92 (br dd, J=4.6, 10.9 Hz, 0.4H), 4.63 (br dd, J=4.5, 10.6 Hz, 0.5H), 3.77 (s, 3H), 3.35-3.23 (m, 1H), 3.20-3.01 (m, 1H), 2.80-2.64 (m, 3H), 1.43-1.33 (m, 9H). LCMS: m/z [M-Boc+H]⁺=277.9, 379.9

### Procedure for preparation of methyl 2-[tert-butoxycarbonyl(methyl)amino]-3-(4,5-dibromo-3-thienyl)propanoate (4)

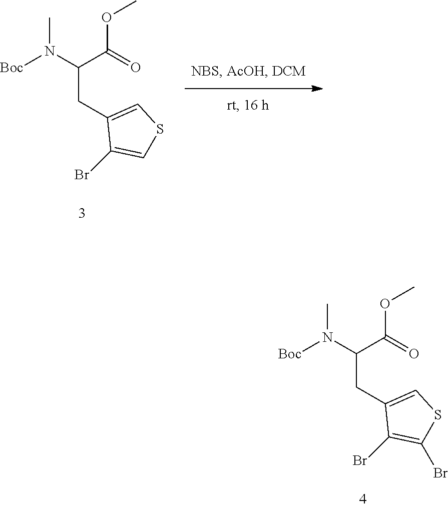 [SMILES: COC(=O)C(Cc1cscc1Br)N(C)C(=O)OC(C)(C)C ; COC(=O)C(Cc1csc(Br)c1Br)N(C)C(=O)OC(C)(C)C]

To a solution of methyl 3-(4-bromo-3-thienyl)-2-[tert-butoxycarbonyl(methyl)amino]propanoate (3) (5 g, 13.22 mmol, 1 eq) and AcOH (1.6 g, 26.44 mmol, 1.51 mL, 2 eq) in DCM (100 mL) was added NBS (2.35 g, 13.22 mmol, 1 eq) slowly at 0° C. The mixture was then stirred at rt for 16 hrs. The reaction mixture was then concentrated under reduced pressure to give a residue. The crude product was purified by reversed-phase HPLC (0.1% FA condition) to give methyl 2-[tert-butoxycarbonyl(methyl)amino]-3-(4,5-dibromo-3-thienyl)propanoate (4) (3 g, 6.04 mmol, 45.67% yield) as a light brown oil. LCMS: m/z [M-Boc+H]⁺=355.8

### Procedure for preparation of methyl 2-[tert-butoxycarbonyl(methyl)amino]-3-(4,5-dibromo-2-iodo-3-thienyl)propanoate (5)

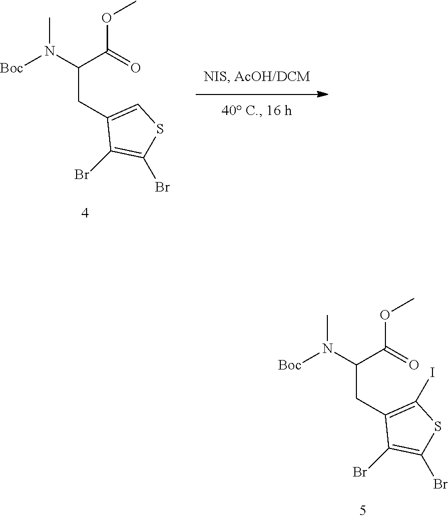 [SMILES: COC(=O)C(Cc1csc(Br)c1Br)N(C)C(=O)OC(C)(C)C ; COC(=O)C(Cc1c(I)sc(Br)c1Br)N(C)C(=O)OC(C)(C)C]

To a solution of methyl 2-[tert-butoxycarbonyl(methyl)amino]-3-(4,5-dibromo-3-thienyl)propanoate (4) (3 g, 6.56 mmol, 1 eq) in DCM (15 mL) and AcOH (15 mL) was added NIS (2.95 g, 13.12 mmol, 2 eq). The mixture was stirred at 40° C. for 16 hrs. The reaction mixture was then cooled and quenched with aq. saturated NaHCO₃ (100 mL), and extracted with EtOAc (50 mL*3). The combined organic layers were washed with brine (100 mL), dried over anhydrous Na₂SO₄, filtered and concentrated under reduced pressure. The residue was purified by flash silica gel chromatography (ISCO®; 40 g SepaFlash® Silica Flash Column, eluent 0~15% Ethyl acetate/Petroleum ether gradient @30 mL/min) to give methyl 2-[tert-butoxycarbonyl(methyl)amino]-3-(4,5-dibromo-2-iodo-3-thienyl)propanoate (5) (3.4 g, 5.83 mmol, 88.86% yield) as a light yellow oil. ¹H NMR (400 MHz, CHLOROFORM-d) δ=4.67-4.55 (m, 1H), 3.83-3.73 (m, 3H), 3.37-3.07 (m, 2H), 2.85-2.74 (m, 3H), 1.46-1.32 (m, 9H). LCMS: m/z [M-Boc+H]⁺=481.7

### Procedure for preparation of methyl 2-[tert-butoxycarbonyl(methyl)amino]-3-[4,5-dibromo-2-(trifluoromethyl)-3-thienyl]propanoate (6)

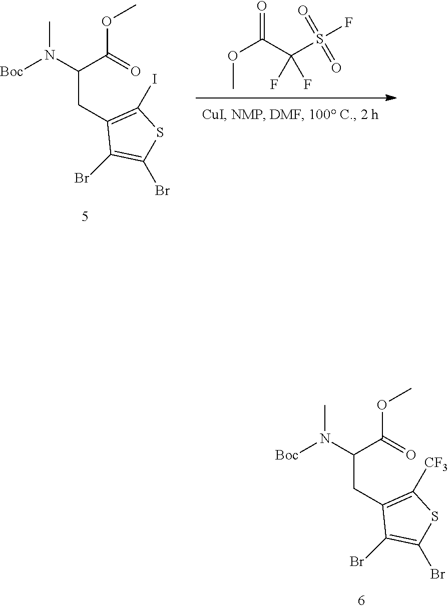 [SMILES: COC(=O)C(Cc1c(I)sc(Br)c1Br)N(C)C(=O)OC(C)(C)C ; COC(=O)C(F)(F)S(=O)(=O)F ; COC(=O)C(Cc1c(C(F)(F)F)sc(Br)c1Br)N(C)C(=O)OC(C)(C)C]

To a solution of methyl 2-[tert-butoxycarbonyl(methyl)amino]-3-(4,5-dibromo-2-iodo-3-thienyl)propanoate (5) (3.4 g, 5.83 mmol, 1 eq) and methyl 2,2-difluoro-2-fluorosulfonyl-acetate (4.5 g, 23.32 mmol, 3 mL, 4 eq) in DMF (30 mL) was added CuI (1.22 g, 6.41 mmol, 1.1 eq) and NMP (2.89 g, 29.16 mmol, 2.83 mL, 5 eq). The mixture was stirred at 100° C. for 2 hr under N₂. The reaction mixture was then cooled and diluted with H₂O (100 mL) and extracted with EtOAc (100 mL*2). The combined organic layers were washed with brine (100 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~15% Ethyl acetate/Petroleum ether gradient @45 mL/min) to give methyl 2-[tert-butoxycarbonyl(methyl)amino]-3-[4,5-dibromo-2-(trifluoromethyl)-3-thienyl]propanoate (6) (2.8 g, 5.33 mmol, 91.43% yield) as a yellow oil. ¹H NMR (400 MHz, CHLOROFORM-d) δ=4.73-4.54 (m, 1H), 3.84-3.70 (m, 3H), 3.50-3.25 (m, 2H), 2.86-2.70 (m, 3H), 1.48-1.32 (m, 9H). LCMS: m/z [M-Boc+H]⁺=423.8

### Procedure for preparation of tert-butyl N-[1-[[4-bromo-2-(trifluoromethyl)-3-thienyl]methyl]-2-hydroxy-ethyl]-N-methyl-carbamate (7)

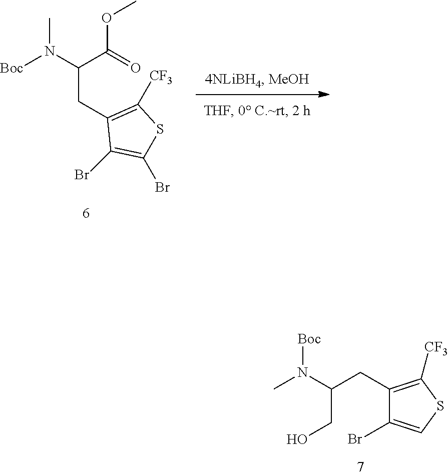 [SMILES: COC(=O)C(Cc1c(C(F)(F)F)sc(Br)c1Br)N(C)C(=O)OC(C)(C)C ; CN(C(=O)OC(C)(C)C)C(CO)Cc1c(Br)csc1C(F)(F)F]

To a solution of methyl 2-[tert-butoxycarbonyl(methyl)amino]-3-[4,5-dibromo-2-(trifluoromethyl)-3-thienyl]propanoate (6) (1 g, 1.90 mmol, 1 eq) in THF (10 mL) was added LiBH₄ (4 M in THF, 1.90 mL, 4 eq) dropwise at 0° C. under N₂. Then MeOH (0.5 mL) was added dropwise at 0° C. The mixture was stirred at rt for 2 hrs. The reaction mixture was quenched by addition aq. sat. NH₄Cl (20 mL) at 0° C., and extracted with EtOAc (10 mL*3). The combined organic layers were washed with brine (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~25% Ethyl acetate/Petroleum ether gradient @30 mL/min) to give tert-butyl N-[1-[[4-bromo-2-(trifluoromethyl)-3-thienyl]methyl]-2-hydroxy-ethyl]-N-methyl-carbamate (7) (660 mg, 915.20 μmol, 48.06% yield) as a yellow solid. LCMS: m/z [M-Boc+H]⁺=318.0, 320.0

### Procedure for preparation of tert-butyl N-methyl-N-[5-(trifluoromethyl)-3,4-dihydro-2H-thieno[3,4-b]pyran-3-yl]carbamate enantiomer 1 (9a) and enantiomer 2 (9b)

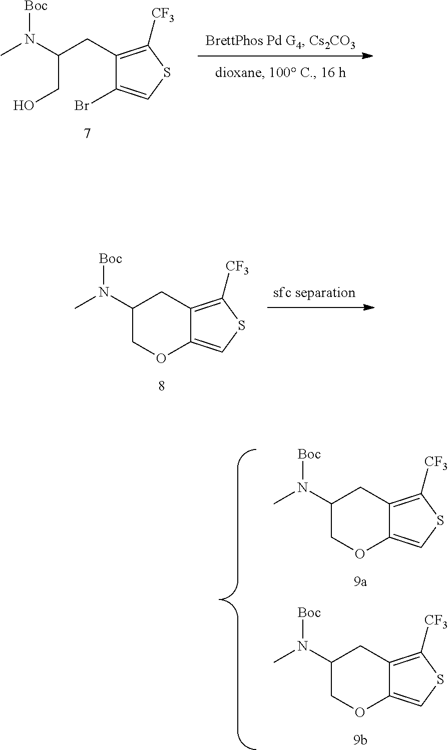 [SMILES: CN(C(=O)OC(C)(C)C)C(CO)Cc1c(Br)csc1C(F)(F)F ; CN(C(=O)OC(C)(C)C)C1COc2csc(C(F)(F)F)c2C1 ; CN(C(=O)OC(C)(C)C)C1COc2csc(C(F)(F)F)c2C1 ; CN(C(=O)OC(C)(C)C)C1COc2csc(C(F)(F)F)c2C1]

To a solution of tert-butyl N-[1-[[4-bromo-2-(trifluoromethyl)-3-thienyl]methyl]-2-hydroxy-ethyl]-N-methyl-carbamate (7) (560 mg, 776.53 μmol, 1 eq) in dioxane (50 mL) was added BrettPhos Pd G4 (71 mg, 77.65 μmol, 0.1 eq) and Cs₂CO₃ (253 mg, 776.53 μmol, 1 eq) under N₂. The mixture was stirred at 100° C. for 16 hrs. The reaction mixture was then cooled and filtered and the filtrate was concentrated under reduced pressure to give a residue. The residue was purified by flash silica gel chromatography (ISCO®; 12 g SepaFlash® Silica Flash Column, eluent 0~10% Ethyl acetate/Petroleum ether gradient @30 mL/min) to give a yellow oil, then the oil was purified by prep-HPLC (column: Waters Xbridge 150*25 mm*5 μm; mobile phase: [water (ammonia hydroxide v/v)-ACN]; gradient:50%-80% B over min) to give racemic tert-butyl N-methyl-N-[5-(trifluoromethyl)-3,4-dihydro-2H-thieno[3,4-b]pyran-3-yl]carbamate (8) (70 mg, 207.50 μmol, 26.72% yield) as a yellowish oil. ¹H NMR (400 MHz, CHLOROFORM-d) δ=6.52 (s, 1H), 4.63-4.30 (m, 1H), 4.13 (br d, J=3.6 Hz, 2H), 3.19-3.08 (m, 1H), 3.06-2.94 (m, 1H), 2.80 (s, 3H), 1.49 (s, 9H). LCMS: m/z [M-tBu+H]⁺=281.9 The enantiomers were obtained from the racemate by SFC separation (Column: DAICEL CHIRALPAK IG (250 mm*50 mm, 10 μm); mobile phase: [CO₂-i-PrOH HEXANE (0.1% NH₃H₂O)]; B %:10%, isocratic elution mode). tert-Butyl N-methyl-N-[5-(trifluoromethyl)-3,4-dihydro-2H-thieno[3,4-b]pyran-3-yl]carbamate enantiomer 1 (9a) (30 mg, 88.93 μmol, 42.86% yield) obtained as a yellow oil. tert-Butyl N-methyl-N-[5-(trifluoromethyl)-3,4-dihydro-2H-thieno[3,4-b]pyran-3-yl]carbamate enantiomer 2 (9b) (25 mg, 74.11 μmol, 35.71% yield) obtained as a yellow oil.

### Procedure for preparation of N-methyl-5-(trifluoromethyl)-3,4-dihydro-2H-thieno[3,4-b]pyran-3-amine hydrochloride enantiomer 1 (258)

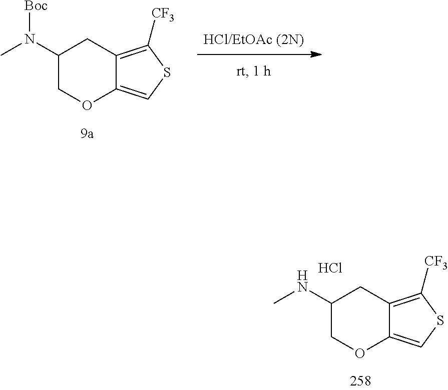 [SMILES: CN(C(=O)OC(C)(C)C)C1COc2csc(C(F)(F)F)c2C1 ; CNC1COc2csc(C(F)(F)F)c2C1 ; Cl]

To a solution of tert-butyl N-methyl-N-[5-(trifluoromethyl)-3,4-dihydro-2H-thieno[3,4-b]pyran-3-yl]carbamate enantiomer 1 (9a) (30 mg, 88.93 μmol, 1 eq) in EtOAc (0.5 mL) was added HCl/EtOAc (0.5 mL, 4N). The mixture was stirred at rt for 1 hr and then concentrated under reduced pressure. The crude product was triturated with EtOAc (0.5 mL) at 20° C. for 5 min, filtered and dried under reduced pressure to give N-methyl-5-(trifluoromethyl)-3,4-dihydro-2H-thieno[3,4-b]pyran-3-amine hydrochloride enantiomer 1 (258) as a white solid. ¹H NMR (400 MHz, METHANOL-d4) δ=6.94 (s, 1H), 4.53 (td, J=2.7, 12.7 Hz, 1H), 4.26 (dd, J=1.1, 12.7 Hz, 1H), 3.79 (br d, J=2.0 Hz, 1H), 3.46-3.36 (m, 1H), 3.28-3.16 (m, 1H), 2.82 (s, 3H). LCMS: m/z [M+H]⁺=282

### Procedure for preparation of N-methyl-5-(trifluoromethyl)-3,4-dihydro-2H-thieno[3,4-b]pyran-3-amine hydrochloride enantiomer 2 (259)

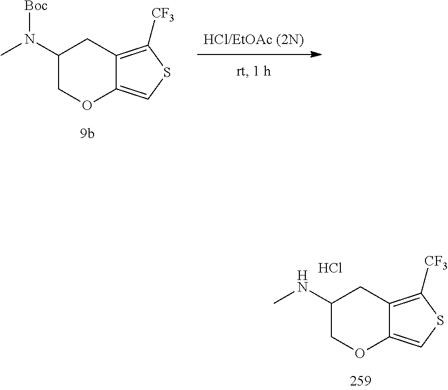 [SMILES: CN(C(=O)OC(C)(C)C)C1COc2csc(C(F)(F)F)c2C1 ; CNC1COc2csc(C(F)(F)F)c2C1 ; Cl]

To a solution of tert-butyl N-methyl-N-[5-(trifluoromethyl)-3,4-dihydro-2H-thieno[3,4-b]pyran-3-yl]carbamate enantiomer 2 (9b) (30 mg, 88.93 μmol, 1 eq) in EtOAc (0.5 mL) was added HCl/EtOAc (0.5 Ml, 4N). The mixture was stirred at rt for 1 hr. The reaction mixture was then concentrated under reduced pressure and the residue was triturated with EtOAc (0.5 mL) at 20° C. for 5 min, filtered and dried under reduced pressure to give N-methyl-5-(trifluoromethyl)-3,4-dihydro-2H-thieno[3,4-b]pyran-3-amine hydrochloride enantiomer 2 (259) as a white solid. ¹H NMR (400 MHz, METHANOL-d4) δ=6.94 (s, 1H), 4.54 (td, J=2.6, 12.6 Hz, 1H), 4.26 (d, J=12.6 Hz, 1H), 3.80 (br d, J=1.6 Hz, 1H), 3.46-3.37 (m, 1H), 3.27-3.19 (m, 1H), 2.82 (s, 3H) LCMS: m/z [M+H]⁺=282

### Synthetic scheme for preparation of N-methyl-7-(trifluoromethyl)-3,4-dihydro-2H-thieno[3,4-b]pyran-3-amine hydrochloride (260)

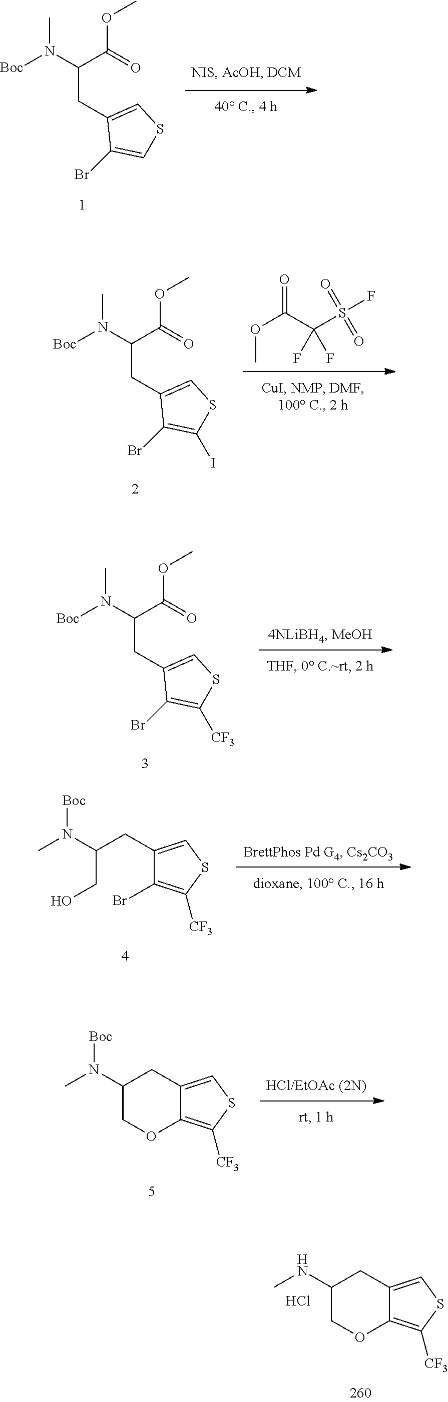

### Procedure for preparation of methyl 3-(4-bromo-5-iodo-3-thienyl)-2-[tert-butoxycarbonyl(methyl)amino]propanoate (2)

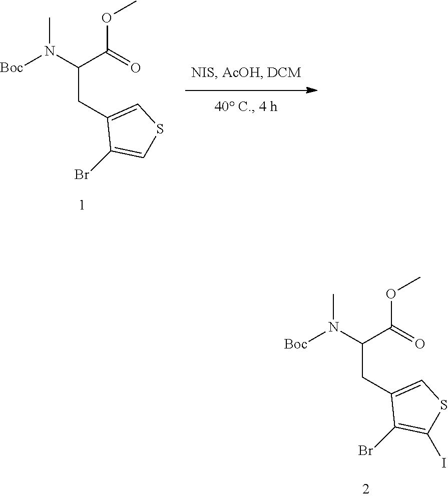 [SMILES: COC(=O)C(Cc1cscc1Br)N(C)C(=O)OC(C)(C)C ; COC(=O)C(Cc1csc(I)c1Br)N(C)C(=O)OC(C)(C)C]

To a solution of methyl 3-(4-bromo-3-thienyl)-2-[tert-butoxycarbonyl(methyl)amino]propanoate (1) (500 mg, 1.32 mmol, 1 eq) and AcOH (158.75 mg, 2.64 mmol, 151.33 μL, 2 eq) in DCM (10 mL) was added NIS (297.38 mg, 1.32 mmol, 1 eq). The mixture was stirred at 40° C. for 4 hrs. The reaction mixture was then cooled and quenched by addition aq. sat. NaHCO₃ (5 mL), and extracted with EtOAc (5 mL*2). The combined organic layers were washed with brine (5 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~20% Ethyl acetate/Petroleum ether gradient @30 mL/min) to give methyl 3-(4-bromo-5-iodo-3-thienyl)-2-[tert-butoxycarbonyl(methyl)amino]propanoate (2) (270 mg, 535.53 μmol, 40.52% yield) as a light brown oil. LCMS: m/z [M-Boc+H]⁺=403.8

### Procedure for preparation of methyl 3-[4-bromo-5-(trifluoromethyl)-3-thienyl]-2-[tert-butoxycarbonyl(methyl)amino]propanoate (3)

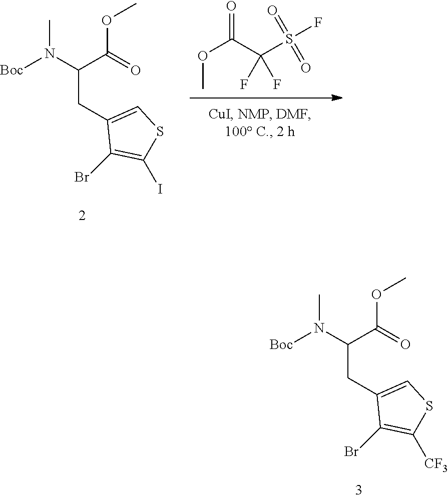 [SMILES: COC(=O)C(Cc1csc(I)c1Br)N(C)C(=O)OC(C)(C)C ; COC(=O)C(F)(F)S(=O)(=O)F ; COC(=O)C(Cc1csc(C(F)(F)F)c1Br)N(C)C(=O)OC(C)(C)C]

To a solution of methyl 3-(4-bromo-5-iodo-3-thienyl)-2-[tert-butoxycarbonyl(methyl)amino]propanoate (2) (270.00 mg, 535.53 μmol, 1 eq) and methyl 2,2-difluoro-2-fluorosulfonyl-acetate (514 mg, 2.68 mmol, 340.67 μL, 5 eq) in DMF (8 mL) was added CuI (112 mg, 589.08 μmol, 1.1 eq) and NMP (265 mg, 2.68 mmol, 260.23 μL, 5 eq). The mixture was stirred at 100° C. for 2 hrs under N₂. The reaction mixture was then cooled and diluted with H₂O (20 mL) and extracted with EtOAc (10 mL*2). The combined organic layers were washed with brine (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~15% Ethyl acetate/Petroleum ether gradient @30 mL/min) to give methyl 3-[4-bromo-5-(trifluoromethyl)-3-thienyl]-2-[tert-butoxycarbonyl(methyl)amino]propanoate (3) (130 mg, 267.99 μmol, 50.04% yield) as a yellow oil. LCMS: m/z [M-Boc+H]⁺=345.9

### Procedure for preparation of tert-butyl N-[1-[[4-bromo-5-(trifluoromethyl)-3-thienyl]methyl]-2-hydroxy-ethyl]-N-methyl-carbamate (4)

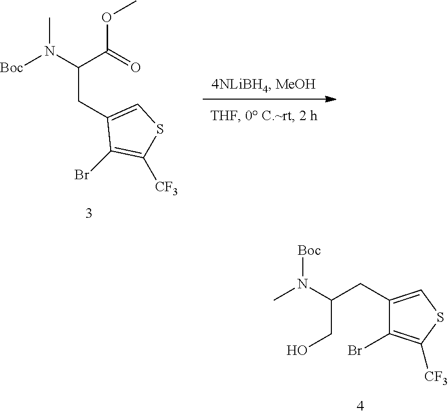 [SMILES: COC(=O)C(Cc1csc(C(F)(F)F)c1Br)N(C)C(=O)OC(C)(C)C ; CN(C(=O)OC(C)(C)C)C(CO)Cc1csc(C(F)(F)F)c1Br]

To a solution of methyl 3-[4-bromo-5-(trifluoromethyl)-3-thienyl]-2-[tert-butoxycarbonyl(methyl)amino]propanoate (3) (110.00 mg, 226.76 μmol, 1 eq) in THF (3 mL) was added LiBH₄ (4 M in THF, 0.34 mL, 6.0 eq) dropwise at 0° C. under N₂, and then MeOH (0.1 mL) also dropwise. The mixture was stirred at 25° C. for 2 hrs and then quenched by adding aq. sat.NH₄Cl (10 mL) at 0° C. dropwise. The mixture was then extracted with EtOAc (5 mL*3). The combined organic layers were washed with brine (10 mL), dried over anhydrous Na₂SO₄, filtered and concentrated under reduced pressure to give tert-butyl N-[1-[[4-bromo-5-(trifluoromethyl)-3-thienyl]methyl]-2-hydroxy-ethyl]-N-methyl-carbamate (4) (100 mg, crude) as a yellow oil. LCMS: m/z [M-Boc+H]⁺=317.9

### Procedure for preparation of tert-butyl N-methyl-N-[7-(trifluoromethyl)-3,4-dihydro-2H-thieno[3,4-b]pyran-3-yl]carbamate (5)

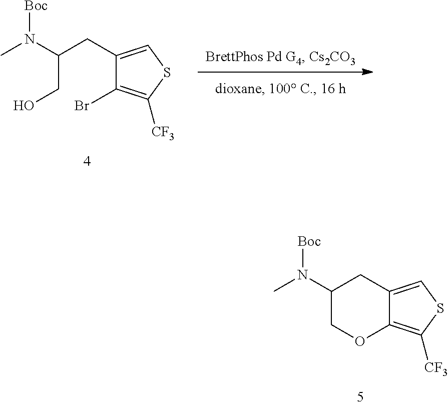 [SMILES: CN(C(=O)OC(C)(C)C)C(CO)Cc1csc(C(F)(F)F)c1Br ; CN(C(=O)OC(C)(C)C)C1COc2c(csc2C(F)(F)F)C1]

To a solution of tert-butyl N-[1-[[4-bromo-5-(trifluoromethyl)-3-thienyl]methyl]-2-hydroxy-ethyl]-N-methyl-carbamate (4) (100.00 mg, 239.08 μmol, 1 eq) in dioxane (10 mL) was added BrettPhos Pd G4 (22 mg, 23.91 μmol, 0.1 eq) and Cs₂CO₃ (156 mg, 478.16 μmol, 2 eq) under N₂. The mixture was stirred at 100° C. for 16 hrs under N₂. The reaction mixture was then cooled, filtered and concentrated under reduced pressure to give a residue. The residue was purified by flash silica gel chromatography (ISCO®; 12 g SepaFlash® Silica Flash Column, eluent 0~10% Ethyl acetate/Petroleum ether gradient @30 mL/min) to give tert-butyl N-methyl-N-[7-(trifluoromethyl)-3,4-dihydro-2H-thieno[3,4-b]pyran-3-yl]carbamate (5) (45 mg, 133.39 μmol, 55.79% yield) as a yellow oil. ¹H NMR (400 MHz, CHLOROFORM-d) δ=7.00 (s, 1H), 4.42 (br s, 1H), 4.28-4.14 (m, 2H), 3.04-2.90 (m, 2H), 2.80 (s, 3H), 1.48 (s, 9H). LCMS: m/z [M-Boc+H]⁺=282.0

### Procedure for preparation of N-methyl-7-(trifluoromethyl)-3,4-dihydro-2H-thieno[3,4-b]pyran-3-amine hydrochloride (260)

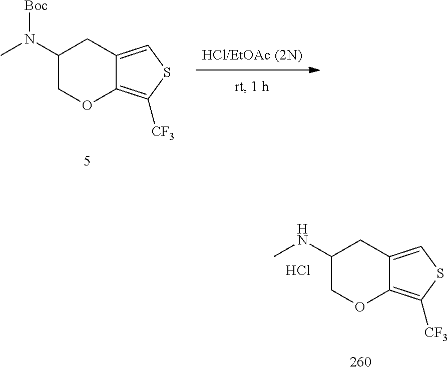 [SMILES: CN(C(=O)OC(C)(C)C)C1COc2c(csc2C(F)(F)F)C1 ; CNC1COc2c(csc2C(F)(F)F)C1 ; Cl]

To a solution of tert-butyl N-methyl-N-[7-(trifluoromethyl)-3,4-dihydro-2H-thieno[3,4-b]pyran-3-yl]carbamate (5) (30.00 mg, 88.93 μmol, 1 eq) in EtOAc (1 mL) was added HCl/EtOAc (1 mL, 4N). The mixture was stirred at 25° C. for 1 hr and then concentrated under reduced pressure to give a residue. The crude product was triturated with EtOAc (1 mL) at 25° C. for 10 min, filtered, collected and dried under reduced pressure to give N-methyl-7-(trifluoromethyl)-3,4-dihydro-2H-thieno[3,4-b]pyran-3-amine hydrochloride (260) (16.67 mg, 59.08 μmol, 66.44% yield) as an off-white solid. ¹H NMR (400 MHz, METHANOL-d₄) δ=7.47 (s, 1H), 4.64-4.53 (m, 1H), 4.36 (dd, J=1.5, 12.6 Hz, 1H), 3.78 (br dd, J=1.6, 3.5 Hz, 1H), 3.30-3.26 (m, 1H), 3.18-3.10 (m, 1H), 2.81 (s, 3H) LCMS: m/z [M+H]⁺=282.0

### Synthetic scheme for preparation of N, 6-dimethyl-3-(trifluoromethyl)-5,7-dihydro-4H-benzothiophen-6-amine hydrochloride enantiomer 1 (261) and enantiomer 2 (262)

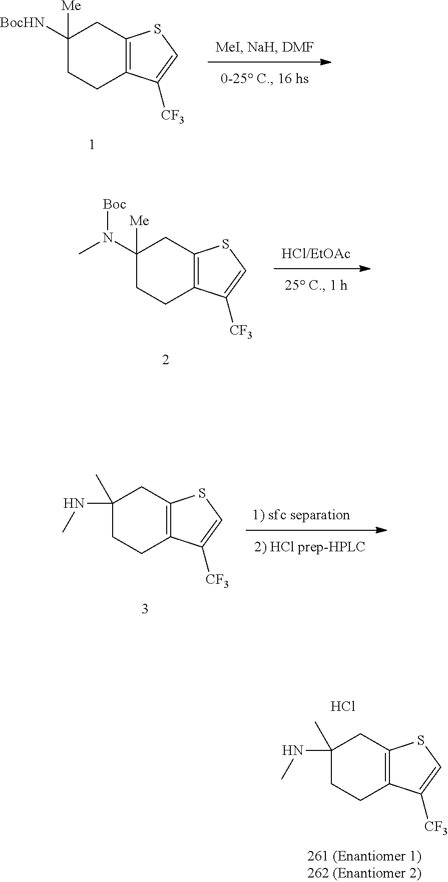 [SMILES: CC1(NC(=O)OC(C)(C)C)CCc2c(C(F)(F)F)csc2C1 ; CN(C(=O)OC(C)(C)C)C1(C)CCc2c(C(F)(F)F)csc2C1 ; CNC1(C)CCc2c(C(F)(F)F)csc2C1 ; Cl ; CNC1(C)CCc2c(C(F)(F)F)csc2C1]

### Procedure for preparation of tert-butyl N-methyl-N-[6-methyl-3-(trifluoromethyl)-5,7-dihydro-4H-benzothiophen-6-yl]carbamate (2)

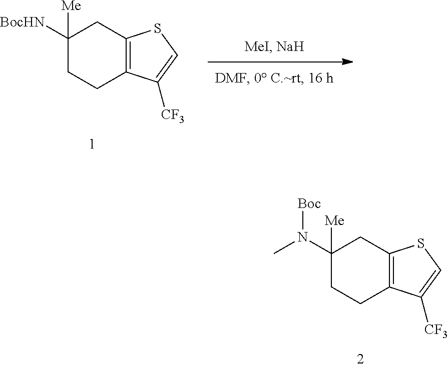 [SMILES: CC1(NC(=O)OC(C)(C)C)CCc2c(C(F)(F)F)csc2C1 ; CN(C(=O)OC(C)(C)C)C1(C)CCc2c(C(F)(F)F)csc2C1]

To a solution of tert-butyl N-[6-methyl-3-(trifluoromethyl)-5,7-dihydro-4H-benzothiophen-6-yl]carbamate (1) (120 mg, 358 μmol, 1.00 eq) in DMF (2 mL) was added NaH (43 mg, 1.07 mmol, 60% suspension in mineral oil, 3.00 eq) at 0° C. The resulting mixture was stirred for 30 min. Then followed addition of MeI (152 mg, 1.07 mmol, 3.00 eq). The mixture was then stirred at room temperature for 15.5 h and then poured into sat. aq. NH₄Cl (10 mL) and water (10 mL). Followed extraction with EtOAc (10 mL*3). The combined organic phase was washed with brine (15 mL), dried over anhydrous sodium sulfate, filtered and concentrated under vacuum to give a residue. The residue was purified by flash silica gel chromatography (ISCO®; 20 g SepaFlash® Silica Flash Column, eluent 0-10% Ethyl acetate/Petroleum ether gradient @50 mL/min) to give tert-butyl N-methyl-N-[6-methyl-3-(trifluoromethyl)-5,7-dihydro-4H-benzothiophen-6-yl]carbamate (112 mg, 321 μmol, 89.6% yield) as a colorless oil. ¹H NMR (400 MHz, CDCl₃-d) 6=7.48 (s, 1H), 3.29 (d, J=16.4 Hz, 1H), 2.95 (br d, J=16.8 Hz, 1H), 2.86 (s, 1H), 2.83-2.66 (m, 6H), 1.80 (td, J=6.4, 13.2 Hz, 1H), 1.47 (s, 9H), 1.43 (s, 3H)). LCMS: m/z [M-tBu+H]⁺=294.1.

### Procedure for preparation of N, 6-dimethyl-3-(trifluoromethyl)-5,7-dihydro-4H-benzothiophen-6-amine hydrochloride enantiomer 1 (261) and enantiomer 2 (262)

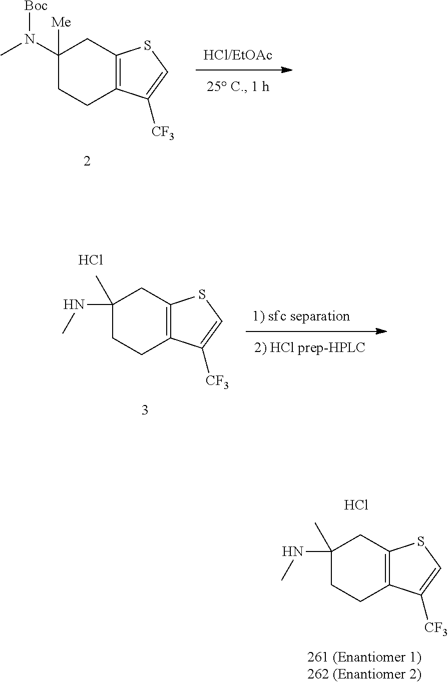 [SMILES: CN(C(=O)OC(C)(C)C)C1(C)CCc2c(C(F)(F)F)csc2C1 ; Cl ; CNC1(C)CCc2c(C(F)(F)F)csc2C1 ; Cl ; CNC1(C)CCc2c(C(F)(F)F)csc2C1]

To a solution of tert-butyl N-methyl-N-[6-methyl-3-(trifluoromethyl)-5,7-dihydro-4H-benzothiophen-6-yl]carbamate (110 mg, 314.82 μmol, 1 eq) in EtOAc (2 mL) was added HCl/EtOAc (4 M, 2 mL). The mixture was stirred at room temperature for 1 h. The reaction mixture was then concentrated under vacuum to give a residue. The crude material was suspended in EtOAc (3 mL), stirred at room temperature for 5 min and then filtered. The filter cake was dried over vacuum to give the crude racemic N, 6-dimethyl-3-(trifluoromethyl)-5,7-dihydro-4H-benzothiophen-6-amine hydrochloride (3). The racemate was separated to its enantiomers by SFC (column: DAICEL CHIRALPAK AD (250 mm*30 mm, 10 μm); mobile phase: [CO₂-EtOH (0.1% NH₃·H₂O)]; B %: 15%, isocratic elution mode) and each enantiomer was further purified by prep-HPLC (column: Phenomenex luna C18 150*25 mm*10 μm; mobile phase: [water(HCl)-ACN]; gradient:5%-35% B over 10 min) to give the products. N, 6-dimethyl-3-(trifluoromethyl)-5,7-dihydro-4H-benzothiophen-6-amine hydrochloride enantiomer 1 (261) (15.41 mg, 53.83 μmol, 25.64% yield, the 1st peak in SFC) was isolated as a white solid. ¹H NMR (400 MHz, MeOD-d₄) δ=7.90 (s, 1H), 3.09 (s, 2H), 2.97-2.80 (m, 2H), 2.84 (s, 3H), 2.11-2.08 (m, 2H), 1.44 (s, 3H). LCMS: m/z [M+H]⁺=250.0. N, 6-dimethyl-3-(trifluoromethyl)-5,7-dihydro-4H-benzothiophen-6-amine hydrochloride enantiomer 2 (262) (17.85 mg, 62.12 μmol, 29.58% yield, the 2nd peak in SFC) was isolated as a white solid. ¹H NMR (400 MHz, MeOD-d₄) δ=7.88 (s, 1H), 3.10 (s, 2H), 2.97-2.80 (m, 2H), 2.85 (s, 3H), 2.11-2.08 (m, 2H), 1.44 (s, 3H). LCMS: m/z [M+H]⁺=250.0.

### Synthetic scheme for preparation of N, 6-dimethyl-5,7-dihydrothieno[3,2-b]pyran-6-amine formate salt (266)

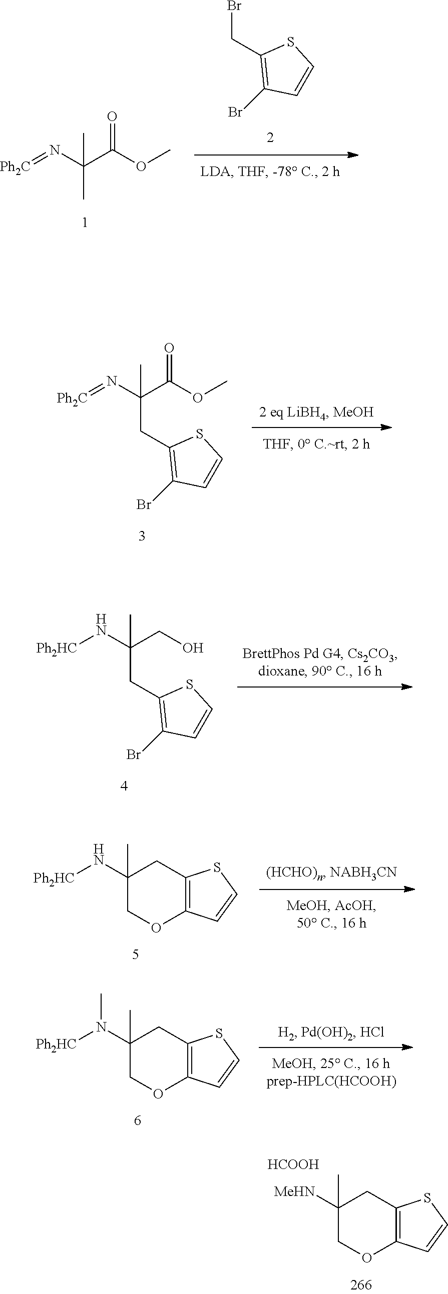

### Procedure for preparation of methyl 2-(benzhydrylideneamino)-3-(3-bromo-2-thienyl)-2-methyl-propanoate (3)

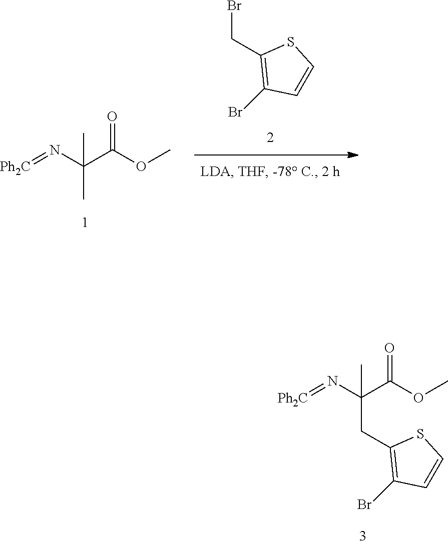

To a solution of LDA (2 M, 37.41 mL, 1.2 eq) in THF (50 mL) was added a solution of methyl 2-(benzhydrylideneamino) propanoate (1) (20 g, 74.82 mmol, 1.2 eq) in THF (100 mL) at −78° C. after stirring for 0.5 h a solution of 3-bromo-2-(bromomethyl)thiophene (15.96 g, 62.35 mmol, 1 eq) in THF (100 mL) was added. The mixture was stirred at −78° C. for 2 hr and under N₂. The reaction mixture was poured in aq. sat. NH₄Cl (100 mL). Followed extraction with EtOAc (50 mL*2). The combined organic layers were washed with aq. sat. NaCl (25 mL*2), dried over Na₂SO₄, filtered and concentrated under reduced pressure to give a residue. The residue was purified by flash silica gel chromatography (ISCO®; 40 g SepaFlash® Silica Flash Column, eluent 0~20% Ethyl acetate/Petroleum ether gradient @60 mL/min). Compound methyl 2-(benzhydrylideneamino)-3-(3-bromo-2-thienyl)-2-methyl-propanoate (3) (27 g, 61.04 mmol, 97.90% yield) was obtained as a yellow oil. LC-MS: m/z [M+H]⁺=442.0 Procedure for synthesis of 2-(benzhydrylamino)-3-(3-bromo-2-thienyl)-2-methyl-propan-1-ol (4)

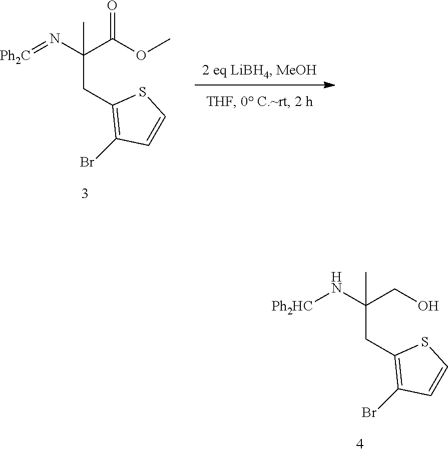

To a solution of methyl 2-(benzhydrylideneamino)-3-(3-bromo-2-thienyl)-2-methyl-propanoate (3) (27 g, 61.04 mmol, 1 eq) in THF (300 mL) was added LiBH₄ (4 M, 61.04 mL, 4 eq) at 0° C. and then MeOH (10 mL). Then the mixture was allowed to naturally warm up to 25° C. and stirred for 16 hr under N₂. The reaction mixture was then quenched with aq. sat. NH₄Cl (200 mL) at 25° C. and extracted with EtOAc (50 mL*4). The combined organic layers were washed with aq. sat. NaCl (50 mL*2), dried over Na₂SO₄, filtered and concentrated under reduced pressure to give a residue. The residue was purified by flash silica gel chromatography (ISCO®; 80 g SepaFlash® Silica Flash Column, eluent 0-25% Ethyl acetate/Petroleum ether gradient @80 mL/min). Compound 2-(benzhydrylamino)-3-(3-bromo-2-thienyl)-2-methyl-propan-1-ol (4) (20 g, 48.03 mmol, 78.70% yield) was obtained as a yellow oil. LC-MS: m/z [M+H]⁺=416.2

### Procedure for preparation of N-benzhydryl-6-methyl-5,7-dihydrothieno[3,2-b]pyran-6-amine (5)

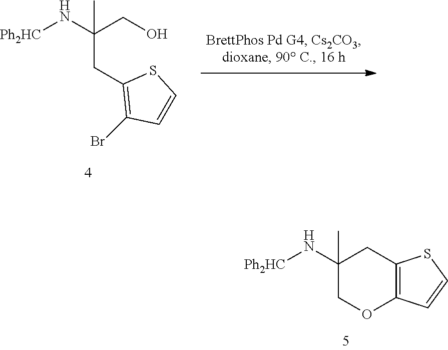

To a solution of 2-(benzhydrylamino)-3-(3-bromo-2-thienyl)-2-methyl-propan-1-ol (4) (2 g, 4.80 mmol, 1 eq) in dioxane (10 mL) was added Brettphos Pd G4 (442.16 mg, 480.34 μmol, 0.1 eq) and cesium carbonate (4.70 g, 14.41 mmol, 3 eq). The mixture was purged with N₂ for 3 times and stirred at 90° C. for 16 hr under N₂ atmosphere. The reaction mixture was then cooled and concentrated under reduced pressure to remove dioxane. The residue was purified by prep-HPLC (neutral condition:column: Waters Xbridge Prep OBD C18 150*40 mm*10 μm; mobile phase: [water(NH₄HCO₃)-ACN]; gradient:55%-85% B over 15 min). Compound N-benzhydryl-6-methyl-5,7-dihydrothieno[3,2-b]pyran-6-amine (5) (1.8 g, 5.37 mmol, 55.85% yield) was obtained as a yellow solid. LC-MS: m/z [M+H]⁺=336.2

### Procedure for preparation of N-benzhydryl-N, 6-dimethyl-5,7-dihydrothieno[3,2-b]pyran-6-amine (6)

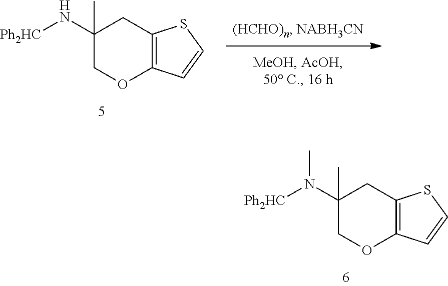

To a solution of N-benzhydryl-6-methyl-5,7-dihydrothieno[3,2-b]pyran-6-amine (5) (1.8 g, 5.37 mmol, 1 eq) in MeOH (20 mL) and AcOH (2 mL) was added formaldehyde (716.05 mg, 23.85 mmol, 656.93 μL, 4 eq) and sodium cyanoborohydride (749.32 mg, 11.92 mmol, 2 eq). The mixture was stirred at 50° C. for 16 hr.

The reaction mixture was then cooled to rt, quenched with aq. sat. NH₄Cl (20 mL) and the mixture, extracted with EtOAc (15 mL*2). The combined organic layers were washed with aq. sat. NaCl (10 mL*2), dried over Na₂SO₄, filtered and concentrated under reduced pressure. The residue was purified by column chromatography (SiO2, Petroleum ether/Ethyl acetate=5/1). Compound N-benzhydryl-N, 6-dimethyl-5,7-dihydrothieno[3,2-b]pyran-6-amine (6) (800 mg, 2.29 mmol, 38.39% yield) was obtained as a white solid. LC-MS: m/z [M+H⁺=350.0

### Procedure for the preparation of N, 6-dimethyl-5,7-dihydrothieno[3,2-b]pyran-6-amine formate salt (266)

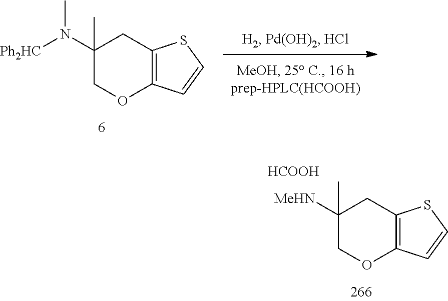

To a solution of N-benzhydryl-N, 6-dimethyl-5,7-dihydrothieno[3,2-b]pyran-6-amine (6) (200 mg, 572.27 μmol, 1 eq) in MeOH (4 mL) was added Pd(OH)₂ (72.65 mg, 258.66 μmol, 50% purity) and HCl (0.1 mL, 36%) under N₂. The suspension was degassed under vacuum and purged with H₂ several times and the mixture was stirred under H₂ (50 psi) at 25° C. for 16 hours. The reaction mixture was then filtered and concentrated under reduced pressure to remove MeOH. The residue was purified by prep-HPLC (column: Waters Atlantis T3 150*30 mm*5 μm; mobile phase: [water(FA)-ACN]; gradient:1%-20% B over 9 min). Compound N, 6-dimethyl-5,7-dihydrothieno[3,2-b]pyran-6-amine formic acid salt (266) (7.88 mg, 42.69 μmol, 7.46% yield) was obtained as a white solid. LC-MS: m/z [M+H+=184.1. ¹H NMR (400 MHz, METHANOL-d₄) δ=8.53 (br s, 1H), 7.24 (d, J=5.5 Hz, 1H), 6.69 (d, J=5.5 Hz, 1H), 4.31 (d, J=12.0 Hz, 1H), 4.01 (d, J=12.1 Hz, 1H), 3.06 (s, 2H), 2.69 (s, 3H), 1.45 (s, 3H)

### Synthetic Scheme for preparation of 3-cyclobutyl-N-methyl-4,5,6,7-tetrahydrobenzothiophen-5-amine hydrochloride enantiomer 1 (267) and enantiomer 2 (268)

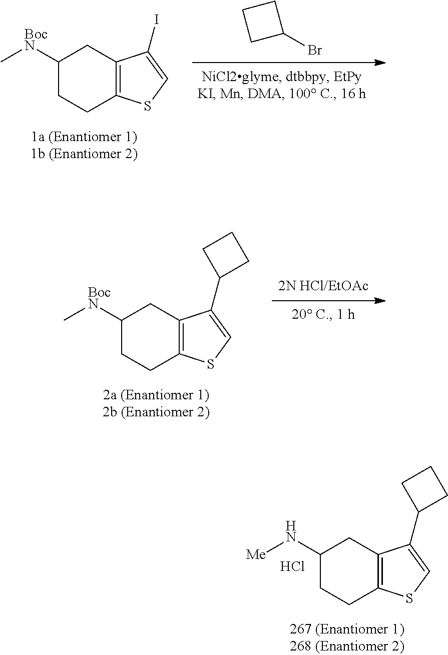 [SMILES: CN(C(=O)OC(C)(C)C)C1CCc2scc(I)c2C1 ; BrC1CCC1 ; CN(C(=O)OC(C)(C)C)C1CCc2scc(C3CCC3)c2C1 ; CNC1CCc2scc(C3CCC3)c2C1 ; Cl]

### Preparation of tert-butyl N-(3-cyclobutyl-4,5,6,7-tetrahydrobenzothiophen-5-yl)-N-methyl-carbamate (2a)

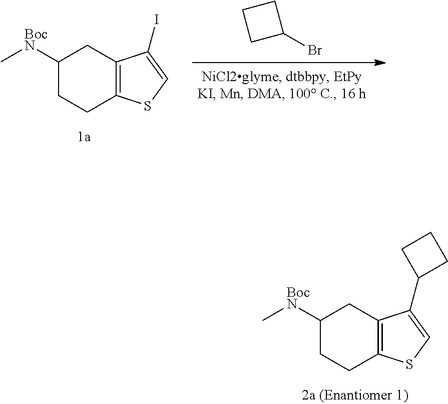 [SMILES: CN(C(=O)OC(C)(C)C)C1CCc2scc(I)c2C1 ; BrC1CCC1 ; CN(C(=O)OC(C)(C)C)C1CCc2scc(C3CCC3)c2C1]

A dried 8 mL vial equipped with a magnetic stirring bar was charged with bromocyclobutane (205.96 mg, 1.53 mmol, 143.63 μL, 3 eq), dichloronickel; 1,2-dimethoxyethane (11.17 mg, 50.85 μmol, 0.1 eq), 4-tert-butyl-2-(4-tert-butyl-2-pyridyl)pyridine (13.65 mg, 50.85 μmol, 0.1 eq), potassium iodide (84.42 mg, 508.54 μmol, 1 eq) and Manganese (55.88 mg, 1.02 mmol, 55.38 μL, 2 eq). Then DMA (2 mL) and 4-ethylpyridine (54.49 mg, 508.54 μmol, 57.85 μL, 1 eq) was added and then tert-butyl N-(3-iodo-4,5,6,7-tetrahydrobenzothiophen-5-yl)-N-methyl-carbamate enantiomer 1 (1a) (200 mg, 508.54 μmol, 1 eq). The resulting mixture was stirred at 100° C. for 16 h. The reaction mixture was then cooled filtered and concentrated under reduced pressure. The residue was purified by prep-HPLC (column: Waters Xbridge 150*25 mm*5 μm; mobile phase: [water(NH₄HCO₃)-ACN]; gradient:70%-100% B over 9 min).Compound tert-butyl N-(3-cyclobutyl-4,5,6,7-tetrahydrobenzothiophen-5-yl)-N-methyl-carbamate enantiomer 1 (2a) (50 mg, 155.53 μmol, 30.58% yield) was obtained as a white solid. LC-MS: m/z [M+H-tBu]⁺=266.0

### Procedure for preparation of tert-butyl N-(3-cyclobutyl-4,5,6,7-tetrahydrobenzothiophen-5-yl)-N-methyl-carbamate (2b)

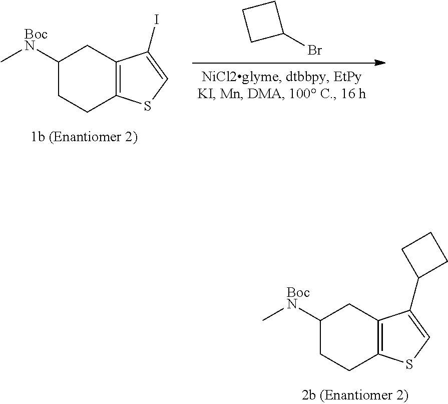 [SMILES: CN(C(=O)OC(C)(C)C)C1CCc2scc(I)c2C1 ; BrC1CCC1 ; CN(C(=O)OC(C)(C)C)C1CCc2scc(C3CCC3)c2C1]

A dried 8 mL vial equipped with a magnetic stirring bar was charged with bromocyclobutane (205.96 mg, 1.53 mmol, 143.63 μL, 3 eq), dichloronickel;1,2-dimethoxyethane (11.17 mg, 50.85 μmol, 0.1 eq), 4-tert-butyl-2-(4-tert-butyl-2-pyridyl)pyridine (13.65 mg, 50.85 μmol, 0.1 eq), potassium iodide (84.42 mg, 508.54 μmol, 1 eq) and Manganese (55.88 mg, 1.02 mmol, 55.38 μL, 2 eq). Then DMA (2 mL) and 4-ethylpyridine (54.49 mg, 508.54 μmol, 57.85 μL, 1 eq) was added and then tert-butyl N-(3-iodo-4,5,6,7-tetrahydrobenzothiophen-5-yl)-N-methyl-carbamate (200 mg, 508.54 μmol, 1 eq). The resulting mixture was stirred at 100° C. for 16 h. The reaction mixture was then cooled, filtered and concentrated under reduced pressure. The residue was purified by prep-HPLC (column: Waters Xbridge 150*25 mm*5 μm; mobile phase: [water(NH₄HCO₃)-ACN]; gradient:70%-100% B over 9 min). Compound tert-butyl N-(3-cyclobutyl-4,5,6,7-tetrahydrobenzothiophen-5-yl)-N-methyl-carbamate enantiomer 2 (2b) (50 mg, 155.53 μmol, 30.58% yield) was obtained as a white solid. LC-MS: m/z [M+H-tBu]⁺=266.0

### Procedure for preparation of 3-cyclobutyl-N-methyl-4,5,6,7-tetrahydrobenzothiophen-5-amine hydrochloride enantiomer 1 (267)

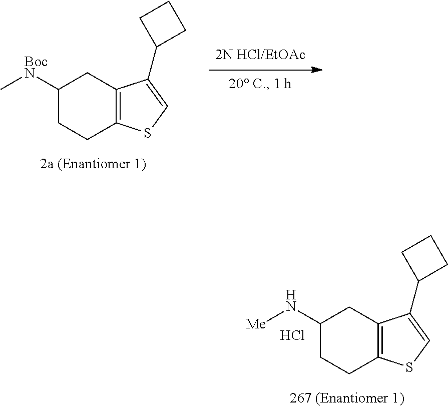 [SMILES: CN(C(=O)OC(C)(C)C)C1CCc2scc(C3CCC3)c2C1 ; CNC1CCc2scc(C3CCC3)c2C1 ; Cl]

To a solution of tert-butyl N-(3-cyclobutyl-4,5,6,7-tetrahydrobenzothiophen-5-yl)-N-methyl-carbamate enantiomer 1 (2a) (40 mg, 124.43 μmol, 1 eq) in EtOAc (2 mL) was added HCl/EtOAc (4 M, 2 mL, 64.30 eq). The mixture was stirred at 20° C. for 1 h and then concentrated under reduced pressure to remove EtOAc. The residue was purified by SFC (condition: column: Phenomenex-Cellulose-2 (250 mm*30 mm, 10 μm); mobile phase: [CO₂-EtOH (0.1% NH₃H₂O)]; B %:40%, isocratic elution mode) and prep-HPLC (HCl condition:column: Phenomenex luna C18 150*25 mm*10 μm; mobile phase: [water(HCl)-ACN]; gradient:10%-40% B over 10 min).Compound 3-cyclobutyl-N-methyl-4,5,6,7-tetrahydrobenzothiophen-5-amine hydrochloride enantiomer 1 (267) (6.10 mg, 22.77 μmol, 14.64% yield) was obtained as a white solid. ¹H NMR (400 MHz, METHANOL-d₄) δ=6.92 (s, 1H), 3.57-3.41 (m, 2H), 3.06-2.91 (m, 3H), 2.81 (s, 3H), 2.53 (br dd, J=9.2, 15.6 Hz, 1H), 2.42-2.28 (m, 3H), 2.20-1.87 (m, 5H). LC-MS: m/z [M+H]⁺=222.1

### Procedure for preparation of 3-cyclobutyl-N-methyl-4,5,6,7-tetrahydrobenzothiophen-5-amine hydrochloride enantiomer 2 (268)

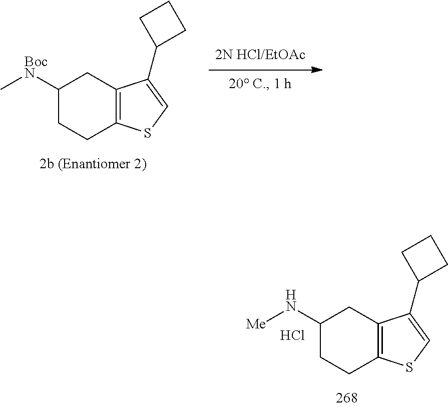 [SMILES: CN(C(=O)OC(C)(C)C)C1CCc2scc(C3CCC3)c2C1 ; CNC1CCc2scc(C3CCC3)c2C1 ; Cl]

To a solution of tert-butyl N-(3-cyclobutyl-4,5,6,7-tetrahydrobenzothiophen-5-yl)-N-methyl-carbamate enantiomer 2 (2b) (40 mg, 124.43 μmol, 1 eq) in EtOAc (2 mL) was added HCl/EtOAc (4 M, 2 mL, 51.44 eq). The mixture was stirred at 20° C. for 1 h and then was concentrated under reduced pressure to remove EtOAc. The residue was purified by prep-HPLC (HCl condition:column: Phenomenex luna C18 150*25 mm*10 μm; mobile phase: [water(HCl)-ACN]; gradient:8%-38% B over 10 min). Compound 3-cyclobutyl-N-methyl-4,5,6,7-tetrahydrobenzothiophen-5-amine hydrochloride enantiomer 2 (268) (17.75 mg, 68.66 μmol, 44.15% yield) was obtained as a white solid. ¹H NMR (400 MHz, METHANOL-d₄) δ=6.91 (s, 1H), 3.56-3.41 (m, 2H), 3.06-2.91 (m, 3H), 2.81 (s, 3H), 2.60-2.51 (m, 1H), 2.43-2.29 (m, 3H), 2.20-1.85 (m, 5H). LC-MS: m/z [M+H+=222.1

### Synthetic scheme for preparation of 1-(6-methyl-5,7-dihydro-4H-benzothiophen-6-yl)pyrrolidine hydrochloride enantiomer 1 (275) and enantiomer 2 (276)

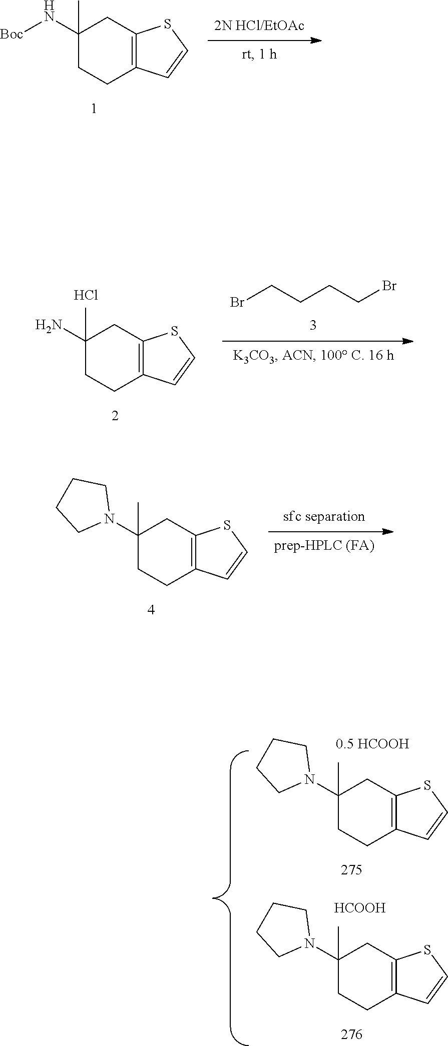

### Preparation of 6-methyl-5,7-dihydro-4H-benzothiophen-6-amine hydrochloride (2)

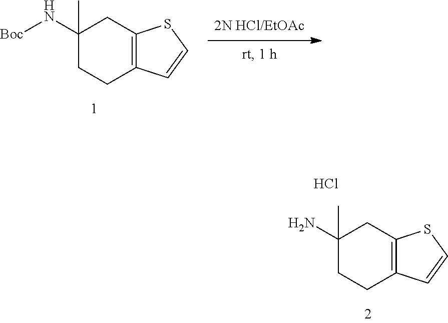 [SMILES: CC1(NC(=O)OC(C)(C)C)CCc2ccsc2C1 ; Cl ; CC1(N)CCc2ccsc2C1]

To a solution of tert-butyl N-(6-methyl-5,7-dihydro-4H-benzothiophen-6-yl)carbamate (1) (500 mg, 1.87 mmol, 1 eq) in EtOAc (1 mL) was added HCl/EtOAc (4 M, 1 mL, 2.14 eq). The mixture was stirred at 25° C. for 1 hr and then was concentrated under reduced pressure to give a residue. The crude product was triturated with ethyl acetate (20 mL) at 25° C. for 30 min and then filtered, dried and collected. Compound 6-methyl-5,7-dihydro-4H-benzothiophen-6-amine hydrochloride (2) (350 mg, 1.72 mmol) was obtained as a white solid. LC-MS: m/z [M-NH2]⁺=150.9

### Procedure for preparation of 1-(6-methyl-4,5,6,7-tetrahydrobenzo[b]thiophen-6-yl)pyrrolidine enantiomer 1 hemiformate salt (275) and enantiomer 2 formate salt (276)

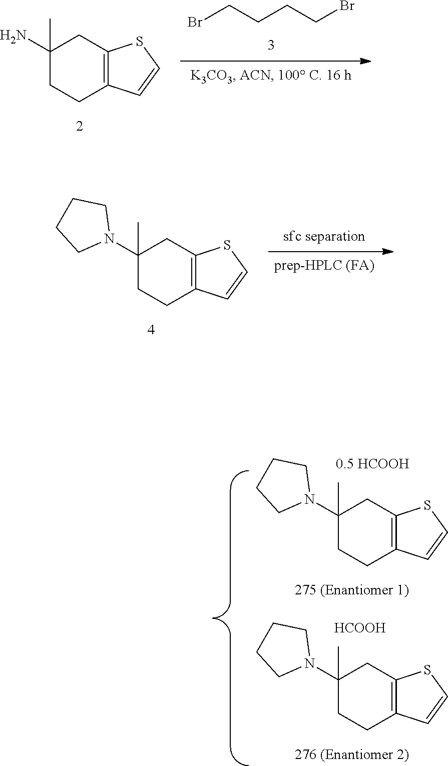

To a solution of racemic 6-methyl-5,7-dihydro-4H-benzothiophen-6-amine hydrochloride (2) (300 mg, 1.47 mmol, 1 eq) and 1,4-dibromobutane (349.73 mg, 1.62 mmol, 195.49 μL, 1.1 eq) in ACN (6 mL) was added K₂CO₃ (610.53 mg, 4.42 mmol, 3 eq) and the mixture was stirred at 100° C. for 16 hr. The reaction mixture was then filtered and concentrated under reduced pressure to give a residue. The residue was purified by flash silica gel chromatography (ISCO®; 40 g SepaFlash® Silica Flash Column, eluent 0~90% Ethyl acetate/Petroleum ether gradient @60 mL/min) to give 1-(6-methyl-4,5,6,7-tetrahydrobenzo[b]thiophen-6-yl)pyrrolidine (4) (300 mg) as a yellow solid. The racemate was separated to its enantiomers by SFC (column: DAICEL CHIRALPAK AD (250 mm*30 mm, 10 μm); mobile phase: [CO₂-i-PrOH (0.1% NH3H2O)]; B %:20%, isocratic elution mode). Enantiomer 1 (Rt=1.089 min in SFC) was further purified by prep-HPLC (column: Phenomenex luna C18 150*25 mm*10 μm; mobile phase: [water(FA)-ACN]; gradient:2%-32% B over 9 min) to provide 1-(6-methyl-4,5,6,7-tetrahydrobenzo[b]thiophen-6-yl)pyrrolidine enantiomer 1 hemiformate salt (275) (43.75 mg, 197.64 μmol, 13.42% yield) as an orange oil. LC-MS: m/z [M+H]⁺=222. ¹H NMR (400 MHz, METHANOL-d4) δ=8.55 (s, 1H), 7.18 (d, J=5.1 Hz, 1H), 6.78 (d, J=5.1 Hz, 1H), 3.17 (br d, J=8.8 Hz, 4H), 3.07-2.90 (m, 2H), 2.88-2.79 (m, 1H), 2.74-2.62 (m, 1H), 2.06-1.90 (m, 6H), 1.26 (s, 3H).Enantiomer 2 (Rt=1.294 min in SFC) was further purified by prep-HPLC (column: YMC-Actus Triart C18 150*30 mm*7 μm; mobile phase: [water(FA)-ACN]; gradient:5%-35% B over 10 min) to provide 1-(6-methyl-4,5,6,7-tetrahydrobenzo[b]thiophen-6-yl)pyrrolidine enantiomer 2 formate salt (276) (86.47 mg, 390.63 μmol, 26.53% yield) as an orange oil. LC-MS: m/z [M+H]⁺=222.1. ¹H NMR (400 MHz, METHANOL-d4) δ=8.53 (s, 1H), 7.23 (d, J=5.1 Hz, 1H), 6.81 (d, J=5.1 Hz, 1H), 3.49-3.35 (m, 4H), 3.17-3.02 (m, 2H), 2.94-2.84 (m, 1H), 2.79-2.68 (m, 1H), 2.16-2.00 (m, 6H), 1.37 (s, 3H).

### Synthetic scheme for preparation of 3-cyclopropyl-4,5,6,7-tetrahydrobenzothiophen-5-amine hydrochloride enantiomer 1 (277) and enantiomer 2 (278)

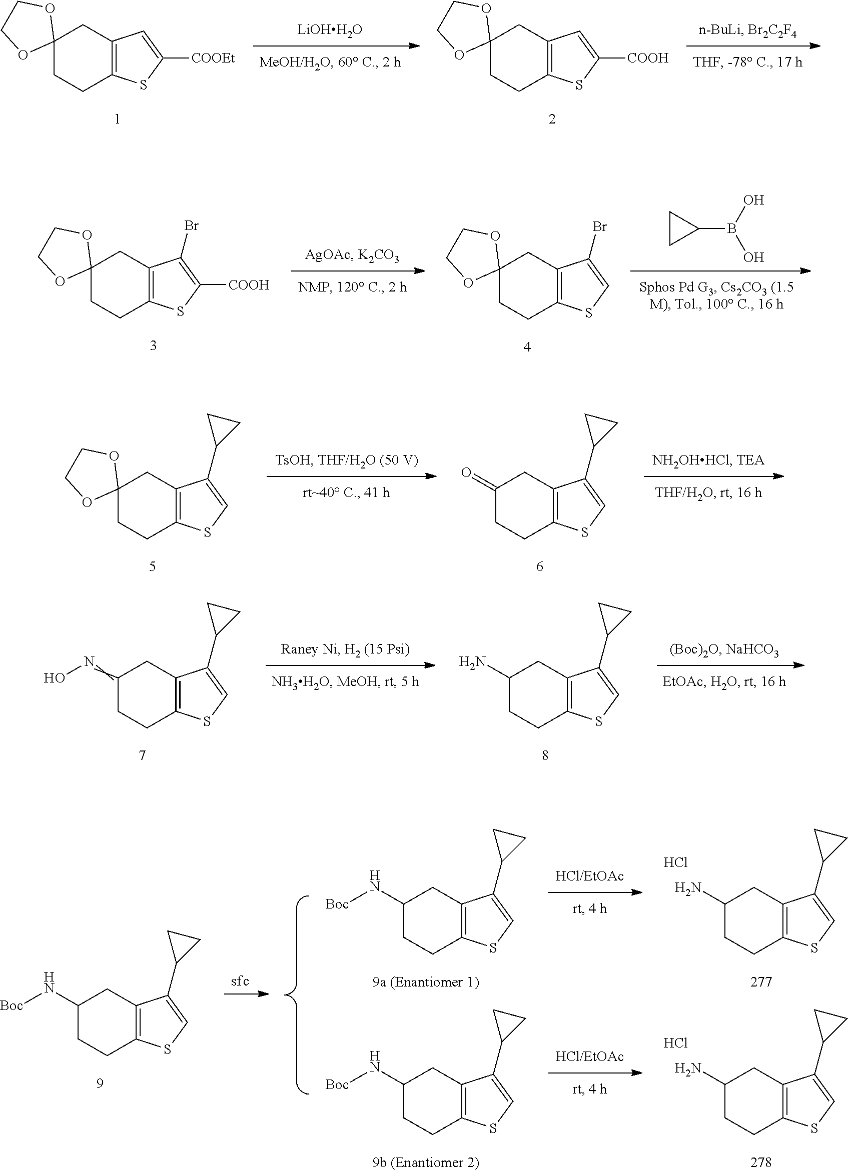

### Procedure for preparation of spiro[1,3-dioxolane-2,5′-6,7-dihydro-4H-benzothiophene]-2′-carboxylic acid (2)

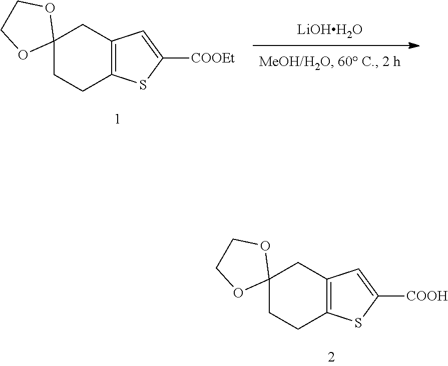 [SMILES: CCOC(=O)c1cc2c(s1)CCC1(C2)OCCO1 ; "LiOH·H2O" ; O=C(O)c1cc2c(s1)CCC1(C2)OCCO1]

To a solution of ethyl spiro[1,3-dioxolane-2,5′-6,7-dihydro-4H-benzothiophene]-2′-carboxylate (1) (5 g, 18.63 mmol, 1 eq) in MeOH (30 mL) was added LiOH·H₂O (2.35 g, 55.90 mmol, 3 eq) and H₂O (30 mL). The mixture was stirred at 60° C. for 2 hr then cooled and 1 M HCl aqueous solution was added into the mixture to adjust the pH to 5. Followed filtration and the filter cake was collected and dried to afford spiro[1,3-dioxolane-2,5′-6,7-dihydro-4H-benzothiophene]-2′-carboxylic acid (2) (3.5 g, 14.57 mmol, 78.17% yield) as a white solid. ¹H NMR (400 MHz, METHANOL-d4) δ=7.41 (s, 1H), 4.05-3.96 (m, 4H), 2.96 (t, J=6.5 Hz, 2H), 2.82 (s, 2H), 1.98 (t, J=6.5 Hz, 2H). LC-MS: m/z [M+H]⁺=241.0

### Procedure for preparation of 3′-bromospiro[1,3-dioxolane-2,5′-6,7-dihydro-4H-benzothiophene]-2′-carboxylic acid (3)

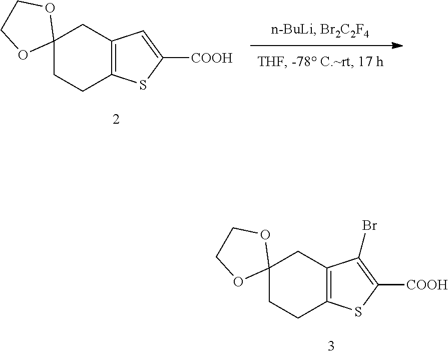

To a solution of spiro[1,3-dioxolane-2,5′-6,7-dihydro-4H-benzothiophene]-2′-carboxylic acid (2) (4.5 g, 18.73 mmol, 1 eq) in THF (80 mL) was added dropwise n-BuLi (2.5 M, 16.48 mL, 2.2 eq) at −78° C. over 0.5 hr. After addition, the mixture was stirred at this temperature for 0.5 hr, and then 1,2-dibromo-1,1,2,2-tetrafluoro-ethane (7.30 g, 28.09 mmol, 1.5 eq) was added dropwise at −78° C. The resulting mixture was allowed to warm up to 25° C., stirred for 16 hr and then quenched slowly by addition NH₄Cl aq. saturated solution (100 mL) at 0° C. and extracted with EtOAc (100 mL*3). The combined organic phases were washed with brine (100 mL), dried over anhydrous sodium sulfate, filtered and concentrated under vacuum to give a residue. The residue was purified by flash silica gel chromatography (ISCO®; 40 g SepaFlash® Silica Flash Column, eluent 0~30% Ethyl acetate/Petroleum ether gradient @60 mL/min) to afford 3′-bromospiro[1,3-dioxolane-2,5′-6,7-dihydro-4H-benzothiophene]-2′-carboxylic acid (3) (9.6 g, crude) as a yellow solid. ¹H NMR (400 MHz, CHLOROFORM-d) δ=4.12-4.00 (m, 4H), 3.02 (t, J=6.6 Hz, 2H), 2.79 (s, 2H), 2.01 (t, J=6.5 Hz, 2H). LC-MS: m/z [M+H]⁺=321.0

### Procedure for preparation of 3′-bromospiro[1,3-dioxolane-2,5′-6,7-dihydro-4H-benzothiophene](4)

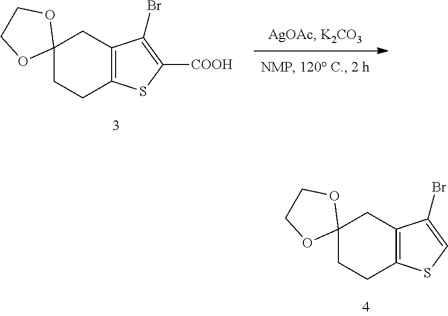

To a solution of 3′-bromospiro[1,3-dioxolane-2,5′-6,7-dihydro-4H-benzothiophene]-2′-carboxylic acid (3) (9.6 g, 30.08 mmol, 1 eq) in NMP (100 mL) was added AgOAc (1.00 g, 6.02 mmol, 0.2 eq) and K₂CO₃ (12.47 g, 90.23 mmol, 3 eq). The mixture was stirred at 120° C. for 2 hr then cooled filtered and the filtrate was poured in H₂O (100 mL), The pH of the mixture was adjusted to ~8 using 1 M HCl aqueous solution. and the mixture was then extracted with EtOAc (100 mL*2). The combined organic phases were washed with brine (50 mL), dried over anhydrous sodium sulfate, filtered and concentrated under vacuum to give a residue. The residue was purified by flash silica gel chromatography (ISCO®; 80 g SepaFlash® Silica Flash Column, eluent 0~10% Ethyl acetate/Petroleum ether gradient @100 mL/min) to afford 3′-bromospiro[1,3-dioxolane-2,5′-6,7-dihydro-4H-benzothiophene](4) (3 g, 10.90 mmol, 36.25% yield) as a colorless liquid. ¹H NMR (400 MHz, CHLOROFORM-d) δ=7.09 (s, 1H), 4.10-4.00 (m, 4H), 2.97 (t, J=6.4 Hz, 2H), 2.75 (s, 2H), 1.98 (t, J=6.5 Hz, 2H)

### Procedure for preparation of 3′-cyclopropylspiro[1,3-dioxolane-2,5′-6,7-dihydro-4H-benzothiophene](5)

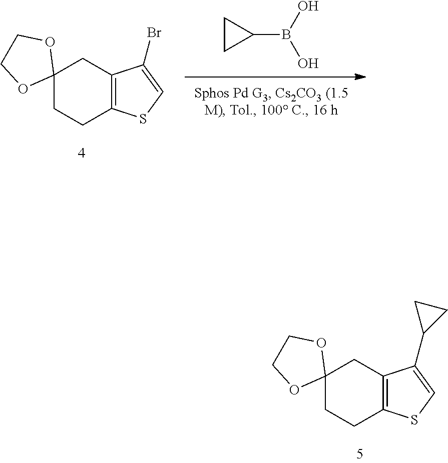 [SMILES: Brc1csc2c1CC1(CC2)OCCO1 ; OB(O)C1CC1 ; c1sc2c(c1C1CC1)CC1(CC2)OCCO1]

A mixture of 3′-bromospiro[1,3-dioxolane-2,5′-6,7-dihydro-4H-benzothiophene](4) (1 g, 3.63 mmol, 1 eq), cyclopropylboronic acid (1.56 g, 18.17 mmol, 5 eq), SPhos Pd G3 (283.56 mg, 363.42 μmol, 0.1 eq), Cs₂CO₃ (1.5 M, 7.27 mL, 3 eq) in Tol. (10 mL) was degassed and purged with N₂ for 3 times, and then the mixture was stirred at 100° C. for 16 hr under N₂ atmosphere. The reaction mixture was then cooled and poured into H₂O (30 mL) and the resulting mixture extracted with EtOAc (20 mL*3). The combined organic phases were washed with brine (20 mL), dried over anhydrous sodium sulfate, filtered and concentrated under vacuum to give a residue. The residue was purified by flash silica gel chromatography (ISCO®; 20 g SepaFlash® Silica Flash Column, eluent 0~10% Ethyl acetate/Petroleum ether gradient @50 mL/min) and by prep-HPLC (column: Phenomenex luna C18 150*40 mm*15 mm; mobile phase: [water(FA)-ACN]; gradient:45%-75% B over 15 min) to afford 3′-cyclopropylspiro[1,3-dioxolane-2,5′-6,7-dihydro-4H-benzothiophene](5) (740 mg, 3.13 mmol, 86.16% yield) as yellow oil. ¹H NMR (400 MHz, CHLOROFORM-d) δ=6.56 (s, 1H), 4.11-4.02 (m, 4H), 2.94 (t, J=6.4 Hz, 2H), 2.87 (s, 2H), 2.00 (t, J=6.5 Hz, 2H), 1.66-1.57 (m, 1H), 0.85-0.76 (m, 2H), 0.60-0.52 (m, 2H). LC-MS: m/z [M+H]⁺=237.2

### Procedure for preparation of 3-cyclopropyl-6,7-dihydro-4H-benzothiophen-5-one (6)

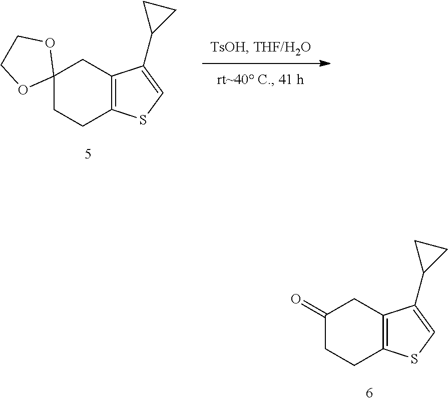 [SMILES: c1sc2c(c1C1CC1)CC1(CC2)OCCO1 ; "TsOH, THF/H2O" ; O=C1CCc2scc(C3CC3)c2C1]

To a solution of 3′-cyclopropylspiro[1,3-dioxolane-2,5′-6,7-dihydro-4H-benzothiophene](5) (740 mg, 3.13 mmol, 1 eq) in THF (20 mL) was added TsOH (1.08 g, 6.26 mmol, 2 eq) and H₂O (20 mL). The mixture was stirred at 25-40° C. for 41 hr. The reaction mixture was then poured into NaHCO₃ aq. saturated solution (50 mL) and extracted with EtOAc (50 mL*3). The combined organic phases were washed with brine (30 mL), dried over anhydrous sodium sulfate, filtered and concentrated under vacuum to give 3-cyclopropyl-6,7-dihydro-4H-benzothiophen-5-one (6) (620 mg, crude) as yellow oil. ¹H NMR (400 MHz, CHLOROFORM-d) δ=6.63 (s, 1H), 3.51 (s, 2H), 3.14 (t, J=6.8 Hz, 2H), 2.73 (t, J=6.8 Hz, 2H), 1.61-1.55 (m, 1H), 0.90-0.80 (m, 2H), 0.62-0.53 (m, 2H). LC-MS: m/z [M+H]⁺=193.0

### Procedure for preparation of 3-cyclopropyl-6,7-dihydro-4H-benzothiophen-5-one oxime (7)

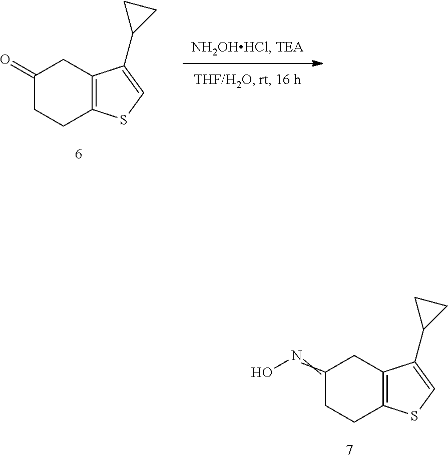 [SMILES: O=C1CCc2scc(C3CC3)c2C1 ; "NH2OH·HCl, TEA" ; ON=C1CCc2scc(C3CC3)c2C1]

To a solution of 3-cyclopropyl-6,7-dihydro-4H-benzothiophen-5-one (6) (620 mg, 3.22 mmol, 1 eq) in THF (8 mL) was added TEA (978.86 mg, 9.67 mmol, 1.35 mL, 3 eq) and NH₂OH·HCl (448.15 mg, 6.45 mmol, 2 eq). The mixture was stirred at 25° C. for 16 hr. The reaction mixture was then poured into H₂O (5 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~18% Ethyl acetate/Petroleum ether gradient @40 mL/min) to afford 3-cyclopropyl-6,7-dihydro-4H-benzothiophen-5-one oxime (7) (600 mg, 2.89 mmol, 89.76% yield) as a pink solid. LC-MS: m/z [M+H]⁺=208.1

### Procedure for preparation of 3-cyclopropyl-4,5,6,7-tetrahydrobenzothiophen-5-amine (8)

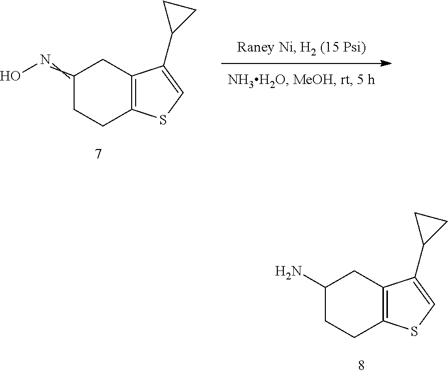 [SMILES: ON=C1CCc2scc(C3CC3)c2C1 ; "Raney Ni, H2 (15 Psi)" ; NC1CCc2scc(C3CC3)c2C1]

To a solution of 3-cyclopropyl-6,7-dihydro-4H-benzothiophen-5-one oxime (7) (550 mg, 2.65 mmol, 1 eq) in MeOH (10 mL) was added aq. NH₃ (34%, 27.35 mg, 265.33 μmol, 30.05 μL, 0.1 eq) and Raney-Ni (56.83 mg, 663.32 μmol, 0.25 eq) under N₂ atmosphere. The suspension was degassed and purged with H₂ for 3 times. The mixture was then stirred under H₂ (15 Psi) at 25° C. for 5 hr and then filtered and the filtrate was concentrated. The residue was purified by prep-HPLC (column: Waters Xbridge Prep OBD C18 150*40 mm*10 μm; mobile phase: [water(NH₄HCO₃)-ACN]; gradient:15%-45% B over 20 min) to afford 3-cyclopropyl-4,5,6,7-tetrahydrobenzothiophen-5-amine (8) (450 mg, 2.33 mmol, 87.74% yield) as yellow oil. LC-MS: m/z [M+H]⁺=194.0

### Procedure for preparation of tert-butyl N-(3-cyclopropyl-4,5,6,7-tetrahydrobenzothiophen-5-yl)carbamate (9)

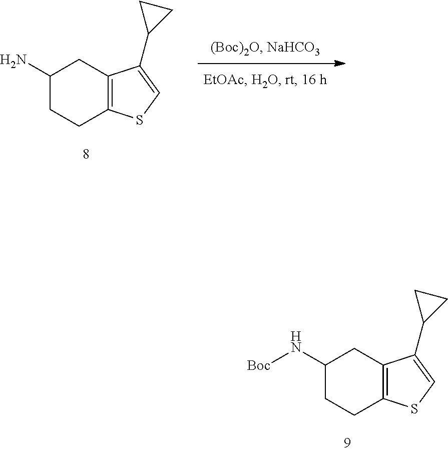

To a solution of 3-cyclopropyl-4,5,6,7-tetrahydrobenzothiophen-5-amine (8) (450 mg, 2.33 mmol, 1 eq) in EtOAc (10 mL) and H₂O (10 mL) was added aq. sat. NaHCO₃ (977.79 mg, 11.64 mmol, 452.89 μL, 5 eq) and (Boc)₂O (762.08 mg, 3.49 mmol, 802.19 μL, 1.5 eq). The mixture was stirred at 25° C. for 16 hr and then extracted with EtOAc (30 mL*2). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~10% Ethyl acetate/Petroleum ether gradient @40 mL/min) to afford tert-butyl N-(3-cyclopropyl-4,5,6,7-tetrahydrobenzothiophen-5-yl)carbamate (9) (440 mg, 1.50 mmol, 64.42% yield) as a white solid. LC-MS: m/z [M+H-tBu]⁺=238.1

### Procedure for preparation of tert-butyl N-(3-cyclopropyl-4,5,6,7-tetrahydrobenzothiophen-5-yl)carbamate enantiomer 1 (9a) and enantiomer 2 (9b)

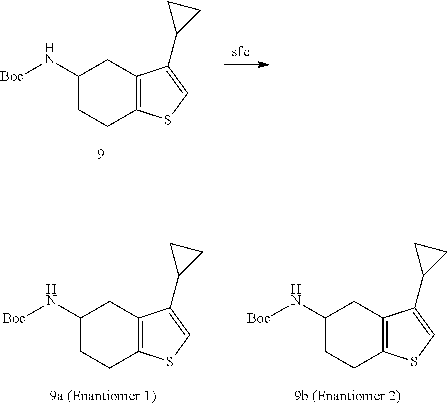 [SMILES: CC(C)(C)OC(=O)NC1CCc2scc(C3CC3)c2C1 ; "sfc" ; CC(C)(C)OC(=O)NC1CCc2scc(C3CC3)c2C1 ; CC(C)(C)OC(=O)NC1CCc2scc(C3CC3)c2C1]

Racemic tert-butyl N-(3-cyclopropyl-4,5,6,7-tetrahydrobenzothiophen-5-yl)carbamate (9) was separated to enantiomers by SFC (column: DAICEL CHIRALPAK IG (250 mm*50 mm, 10 μm); mobile phase: [CO₂-MeOH (0.1% NH₃H₂O)]; B %:15%, isocratic elution mode) to afford tert-butyl N-(3-cyclopropyl-4,5,6,7-tetrahydrobenzothiophen-5-yl)carbamate enantiomer 1 (9a) (200 mg, 681.61 μmol, 45.45% yield) as a white solid and tert-butyl N-(3-cyclopropyl-4,5,6,7-tetrahydrobenzothiophen-5-yl)carbamate enantiomer 2 (9b) (200 mg, 681.61 μmol, 45.45% yield) as a white solid.

### Procedure for preparation of 3-cyclopropyl-4,5,6,7-tetrahydrobenzothiophen-5-amine hydrochloride enantiomer 1 (277)

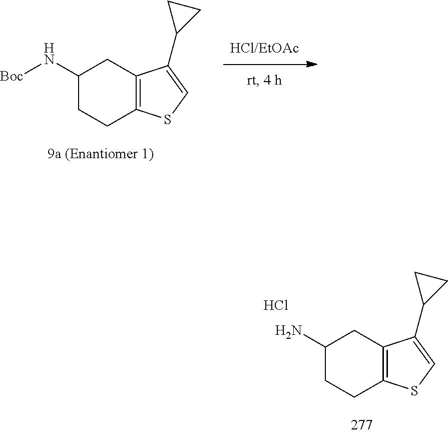 [SMILES: CC(C)(C)OC(=O)NC1CCc2scc(C3CC3)c2C1 ; Cl ; NC1CCc2scc(C3CC3)c2C1]

To a solution of tert-butyl N-(3-cyclopropyl-4,5,6,7-tetrahydrobenzothiophen-5-yl)carbamate enantiomer 1 (9a) (200 mg, 681.61 μmol, 1 eq) in EtOAc (2 mL) was added HCl/EtOAc (4 M, 2 mL, 11.74 eq). The mixture was stirred at 25° C. for 4 hr and thenconcentrated. The residue was suspended in EtOAc at 25° C. for 10 min. The mixture was then filtered and the filter cake was collected and dried to afford 3-cyclopropyl-4,5,6,7-tetrahydrobenzothiophen-5-amine hydrochloride enantiomer 1 (277) (145.50 mg, 626.91 μmol, 91.98% yield) as a white solid. ¹H NMR (400 MHz, METHANOL-d4) δ=6.69 (s, 1H), 3.62 (dddd, J=3.0, 5.6, 8.8, 10.8 Hz, 1H), 3.16 (ddd, J=0.9, 5.3, 15.9 Hz, 1H), 2.93 (br t, J=5.4 Hz, 2H), 2.65 (tdd, J=1.7, 9.1, 15.9 Hz, 1H), 2.29-2.20 (m, 1H), 1.95 (s, 1H), 1.70-1.62 (m, 1H), 0.88-0.80 (m, 2H), 0.58-0.52 (m, 2H). LC-MS: m/z [M+H]⁺=194.1

### Procedure for preparation of 3-cyclopropyl-4,5,6,7-tetrahydrobenzothiophen-5-amine hydrochloride enantiomer 2 (278)

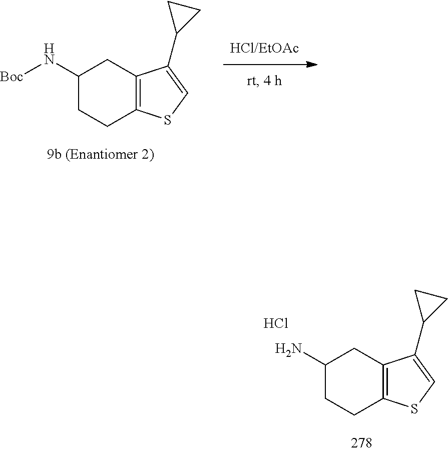 [SMILES: CC(C)(C)OC(=O)NC1CCc2scc(C3CC3)c2C1 ; Cl ; NC1CCc2scc(C3CC3)c2C1]

To a solution of tert-butyl N-(3-cyclopropyl-4,5,6,7-tetrahydrobenzothiophen-5-yl)carbamate enantiomer 2 (9b) (200 mg, 681.61 μmol, 1 eq) in EtOAc (2 mL) was added HCl/EtOAc (4 M, 2 mL, 11.74 eq). The mixture was stirred at 25° C. for 4 hr. The mixture was then concentrated and the residue was suspended in EtOAc at 25° C. for 10 min The suspension was then filtered, the filter cake was collected and dried to afford 3-cyclopropyl-4,5,6,7-tetrahydrobenzothiophen-5-amine hydrochloride enantiomer 2 (278) (147.11 mg, 633.85 μmol, 92.99% yield) as a white solid. ¹H NMR (400 MHz, METHANOL-d4) δ=6.69 (s, 1H), 3.66-3.57 (m, 1H), 3.16 (ddd, J=1.0, 5.4, 15.9 Hz, 1H), 2.93 (br t, J=5.4 Hz, 2H), 2.65 (tdd, J=1.8, 9.0, 15.9 Hz, 1H), 2.29-2.20 (m, 1H), 1.95 (dddd, J=7.1, 8.7, 10.7, 12.6 Hz, 1H), 1.71-1.62 (m, 1H), 0.90-0.79 (m, 2H), 0.59-0.51 (m, 2H). LC-MS: m/z [M+H]⁺=194.1

### Synthetic scheme for preparation of 3-ethyl-4,5,6,7-tetrahydrobenzothiophen-5-amine hydrochloride enantiomer 1 (279) and enantiomer 2 (280)

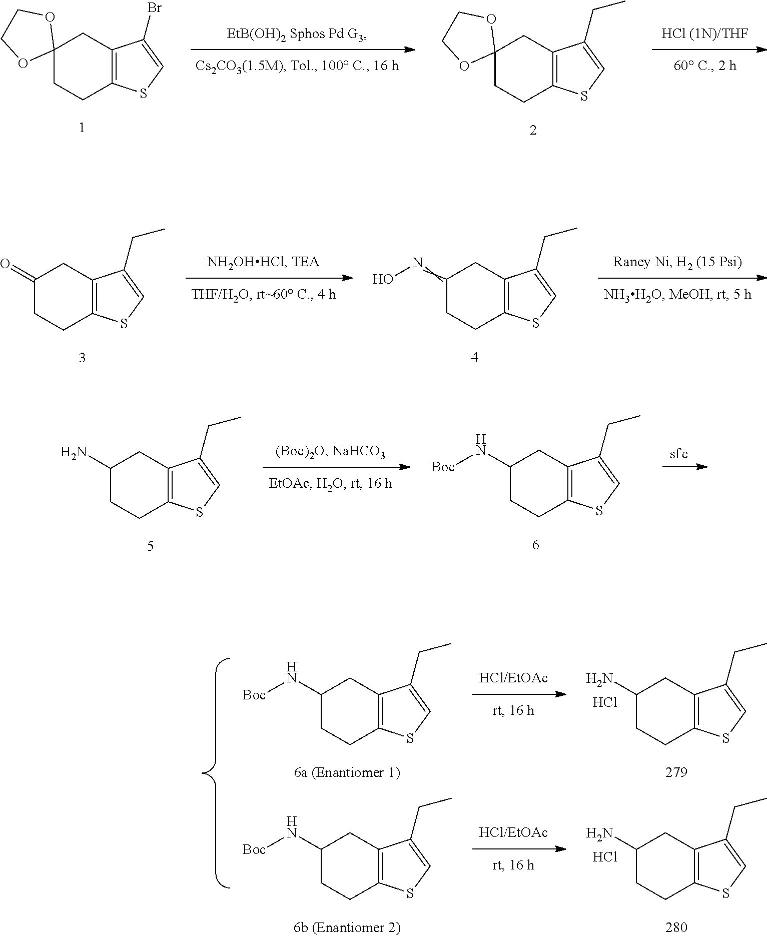

### Procedure for preparation of 3′-ethylspiro[1,3-dioxolane-2,5′-6,7-dihydro-4H-benzothiophene](2)

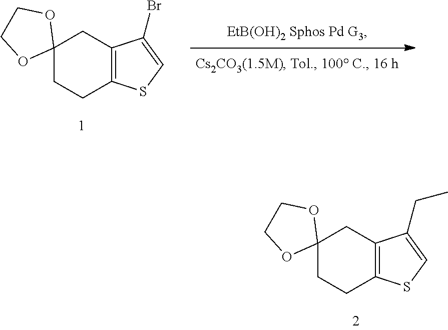

A mixture of 3′-bromospiro[1,3-dioxolane-2,5′-6,7-dihydro-4H-benzothiophene](1) (1 g, 3.63 mmol, 1 eq), ethylboronic acid (1.34 g, 18.17 mmol, 5 eq), SPhos Pd G₃ (283.56 mg, 363.42 μmol, 0.1 eq), Cs₂CO₃ (1.5 M, 7.27 mL, 3 eq) in Tol. (5 mL) was degassed and purged with N₂ for 3 times, and then the mixture was stirred at 100° C. for 16 hr under N₂ atmosphere. The reaction mixture was poured into H₂O (10 mL) and extracted with EtOAc (20 mL*3). The combined organic phases were washed with brine (10 mL), dried over anhydrous sodium sulfate, filtered and concentrated under vacuum to give a residue. The residue was purified by flash silica gel chromatography (ISCO®; 20 g SepaFlash® Silica Flash Column, eluent 0~10% Ethyl acetate/Petroleum ether gradient @40 mL/min) and was purified by prep-HPLC (column: Phenomenex luna C18 150*40 mm*15 μm; mobile phase: [water(FA)-ACN]; gradient:42%-72% B over 15 min) to afford 3′-ethylspiro[1,3-dioxolane-2,5′-6,7-dihydro-4H-benzothiophene](2) (760 mg, 3.39 mmol, 93.23% yield) as a white solid. ¹H NMR (400 MHz, CHLOROFORM-d) δ=6.74 (s, 1H), 4.11-3.98 (m, 4H), 2.96 (t, J=6.4 Hz, 2H), 2.73 (s, 2H), 2.44 (q, J=7.4 Hz, 2H), 1.99 (t, J=6.5 Hz, 2H), 1.22 (t, J=7.4 Hz, 3H). LC-MS: m/z [M+H]⁺=225.2

### Procedure for preparation of 3-ethyl-6,7-dihydro-4H-benzothiophen-5-one (3)

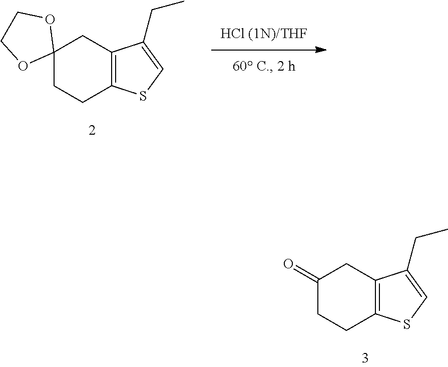

To a solution of 3′-ethylspiro[1,3-dioxolane-2,5′-6,7-dihydro-4H-benzothiophene](2) (760 mg, 3.39 mmol, 1 eq) in THF (10 mL) was added aq. HCl (1 M, 10.13 mL, 2.99 eq). The mixture was stirred at 60° C. for 2 hr. The reaction mixture was then cooled and poured into NaHCO₃ aq. saturated solution (20 mL) and extracted with EtOAc (20 mL*2). The combined organic phases were washed with brine (10 mL), dried over anhydrous sodium sulfate, filtered and concentrated under vacuum to give 3-ethyl-6,7-dihydro-4H-benzothiophen-5-one (3) (660 mg, crude) as yellow oil. ¹H NMR (400 MHz, CHLOROFORM-d) δ=6.81 (s, 1H), 3.37 (s, 2H), 3.15 (br t, J=6.8 Hz, 2H), 2.72 (t, J=6.8 Hz, 2H), 2.46 (q, J=7.5 Hz, 2H), 1.28-1.16 (m, 3H). LC-MS: m/z [M+H]⁺=181.1

### Procedure for preparation of 3-ethyl-6,7-dihydro-4H-benzothiophen-5-one oxime (4)

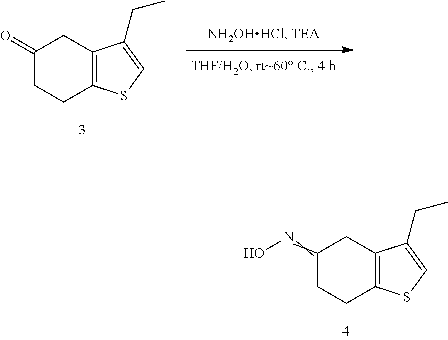

To a solution of 3-ethyl-6,7-dihydro-4H-benzothiophen-5-one (3) (660 mg, 3.66 mmol, 1 eq) in THF (8 mL) was added TEA (1.11 g, 10.98 mmol, 1.53 mL, 3 eq) and NH₂OH·HCl (508.85 mg, 7.32 mmol, 2 eq) at rt. The mixture was then stirred 60° C. for 4 hr. The reaction mixture was then cooled and poured into H₂O (5 mL) and extracted with EtOAc (20 mL*2). The combined organic phases were washed with brine (10 mL), dried over anhydrous sodium sulfate, filtered and concentrated under vacuum to give a residue. The residue was purified by flash silica gel chromatography (ISCO®; 20 g SepaFlash® Silica Flash Column, eluent 0~17% Ethyl acetate/Petroleum ether gradient @40 mL/min) to afford 3-ethyl-6,7-dihydro-4H-benzothiophen-5-one oxime (4) (620 mg, 3.17 mmol, 86.72% yield) as a yellow solid. LC-MS: m/z [M+H]⁺=196.1

### Procedure for preparation of 3-ethyl-4,5,6,7-tetrahydrobenzothiophen-5-amine (4)

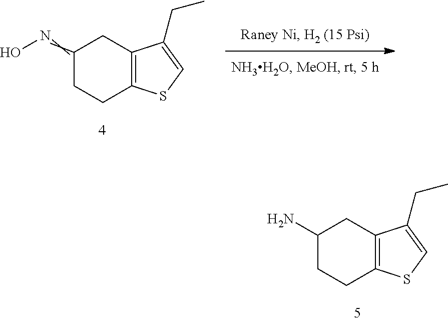

To a solution of 3-ethyl-6,7-dihydro-4H-benzothiophen-5-one oxime (4) (570 mg, 2.92 mmol, 1 eq) and NH₃·H₂O (34%, 30.09 mg, 291.89 μmol, 33.06 μL, 0.1 eq) in MeOH (10 mL) was added Raney-Ni (50.01 mg, 583.77 μmol, 0.2 eq) under N₂ atmosphere. The suspension was degassed and purged with H₂ for 3 times and stirred under H₂ (15 Psi) at 25° C. for 5 hr. Followed filtration and the filtrate was concentrated to give a residue that was purified by prep-HPLC (column: Waters Xbridge Prep OBD C18 150*40 mm*10 μm; mobile phase: [water(NH₄HCO₃)-ACN]; gradient:12%-42% B over 20 min) to afford 3-ethyl-4,5,6,7-tetrahydrobenzothiophen-5-amine (5) (500 mg, 2.76 mmol, 94.49% yield) as a yellow solid. LC-MS: m/z [M-NH₂]⁺=165.1

### Procedure for preparation of tert-butyl N-(3-ethyl-4,5,6,7-tetrahydrobenzothiohen-5-yl)carbamate (6)

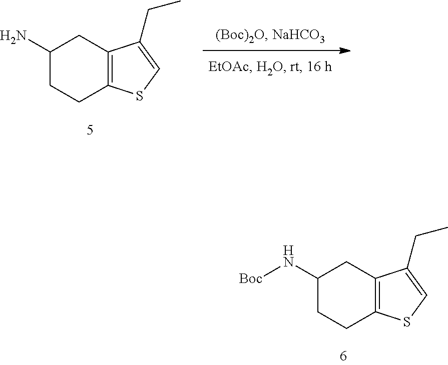

To a solution of 3-ethyl-4,5,6,7-tetrahydrobenzothiophen-5-amine (5) (500 mg, 2.76 mmol, 1 eq) in EtOAc (10 mL) and H₂O (10 mL) was added NaHCO₃ (1.16 g, 13.79 mmol, 5 eq) and (Boc)₂O (902.85 mg, 4.14 mmol, 950.37 μL, 1.5 eq). The mixture was stirred at 25° C. for 16 hr and then extracted with EtOAc (30 mL*2). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~9% Ethyl acetate/Petroleum ether gradient @40 mL/min) to afford tert-butyl N-(3-ethyl-4,5,6,7-tetrahydrobenzothiophen-5-yl)carbamate (6) (590 mg, 2.10 mmol, 76.02% yield) as a yellow solid. LC-MS: m/z [M+H-tBu]⁺=226.0

### Procedure for preparation of tert-butyl N-(3-ethyl-4,5,6,7-tetrahydrobenzothiophen-5-yl)carbamate enantiomer 1 (6a) and enantiomer 2 (6b)

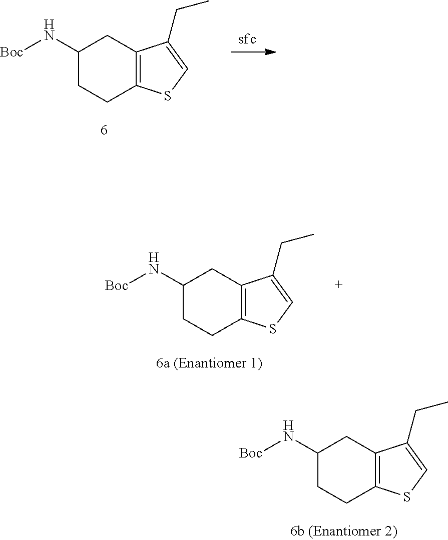 [SMILES: CCc1csc2c1CC(NC(=O)OC(C)(C)C)CC2 ; "sfc" ; CCc1csc2c1CC(NC(=O)OC(C)(C)C)CC2 ; CCc1csc2c1CC(NC(=O)OC(C)(C)C)CC2]

Racemic tert-butyl N-(3-ethyl-4,5,6,7-tetrahydrobenzothiophen-5-yl)carbamate (6) was separated to its enantiomers by SFC (column: DAICEL CHIRALPAK IG (250 mm*50 mm, 10 μm); mobile phase: [CO₂-MeOH (0.1% NH₃H₂O)]; B %:11%, isocratic elution mode) to afford tert-butyl N-(3-ethyl-4,5,6,7-tetrahydrobenzothiophen-5-yl)carbamate enantiomer 1 (6a) (250 mg, 888.37 μmol, 42.37% yield) as a white solid and tert-butyl N-(3-ethyl-4,5,6,7-tetrahydrobenzothiophen-5-yl)carbamate enantiomer 2 (6b) (280 mg, 994.98 μmol, 47.46% yield) as a white solid.

### Procedure for preparation of 3-ethyl-4,5,6,7-tetrahydrobenzothiophen-5-amine hydrochloride enantiomer 1 (279)

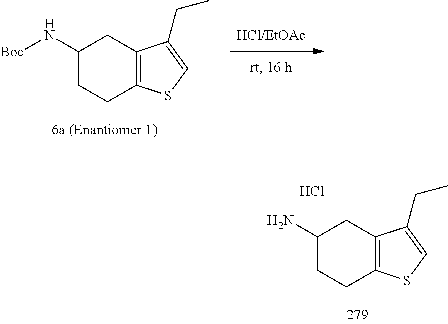 [SMILES: CCc1csc2c1CC(NC(=O)OC(C)(C)C)CC2 ; CCc1csc2c1CC(N)CC2 ; Cl]

To a solution of tert-butyl N-(3-ethyl-4,5,6,7-tetrahydrobenzothiophen-5-yl)carbamate enantiomer 1 (6a) (250 mg, 888.37 μmol, 1 eq) in EtOAc (3 mL) was added HCl/EtOAc (4 M, 2 mL, 9.01 eq). The mixture was stirred at 25° C. for 16 h and then concentrated. The solid residue was suspended in EtOAc at 25° C. for 10 min. The mixture was then filtered, the filter cake was collected and dried to afford 3-ethyl-4,5,6,7-tetrahydrobenzothiophen-5-amine hydrochloride enantiomer 1 (279) (182.48 mg, 829.61 μmol, 93.39% yield) as a white solid. ¹H NMR (400 MHz, METHANOL-d4) δ=6.85 (s, 1H), 3.59 (dddd, J=3.0, 5.5, 8.8, 10.8 Hz, 1H), 3.05-2.85 (m, 3H), 2.59-2.41 (m, 3H), 2.29-2.18 (m, 1H), 2.03-1.87 (m, 1H), 1.22 (t, J=7.5 Hz, 3H). LC-MS: m/z [M+H]⁺=182.0

### Procedure for preparation of 3-ethyl-4,5,6,7-tetrahydrobenzothiophen-5-amine hydrochloride enantiomer 2 (280)

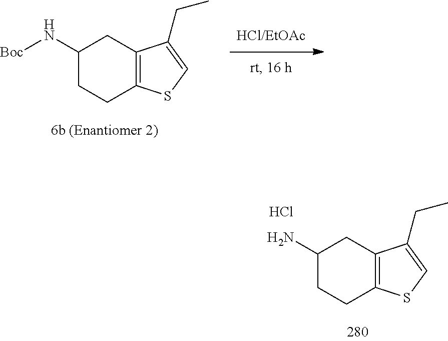 [SMILES: CCc1csc2c1CC(NC(=O)OC(C)(C)C)CC2 ; Cl ; CCc1csc2c1CC(N)CC2]

To a solution of tert-butyl N-(3-ethyl-4,5,6,7-tetrahydrobenzothiophen-5-yl)carbamate enantiomer 2 (6b) (280 mg, 994.98 μmol, 1 eq) in EtOAc (3 mL) was added HCl/EtOAc (4 M, 2.24 mL, 9.01 eq). The mixture was stirred at 25° C. for 16 h and then concentrated. The solid residue was suspended in EtOAc at 25° C. for 10 min. The mixture was filtered, the filter cake was collected and dried to afford 3-ethyl-4,5,6,7-tetrahydrobenzothiophen-5-amine hydrochloride enantiomer 2 (280) (183.15 mg, 832.66 μmol, 83.69% yield) as a white solid. ¹H NMR (400 MHz, METHANOL-d4) δ=6.85 (s, 1H), 3.64-3.54 (m, 1H), 3.05-2.85 (m, 3H), 2.60-2.42 (m, 3H), 2.24 (td, J=4.0, 8.5 Hz, 1H), 1.94 (dddd, J=6.8, 9.0, 10.7, 12.6 Hz, 1H), 1.22 (t, J=7.5 Hz, 3H) LC-MS: m/z [M+H]⁺=182.1

### Synthetic scheme for preparation of 5-methyl-4,5,6,7-tetrahydrobenzo[b]thiophen-5-amine hydrochloride enantiomer 1 (281) and enantiomer 2 (282)

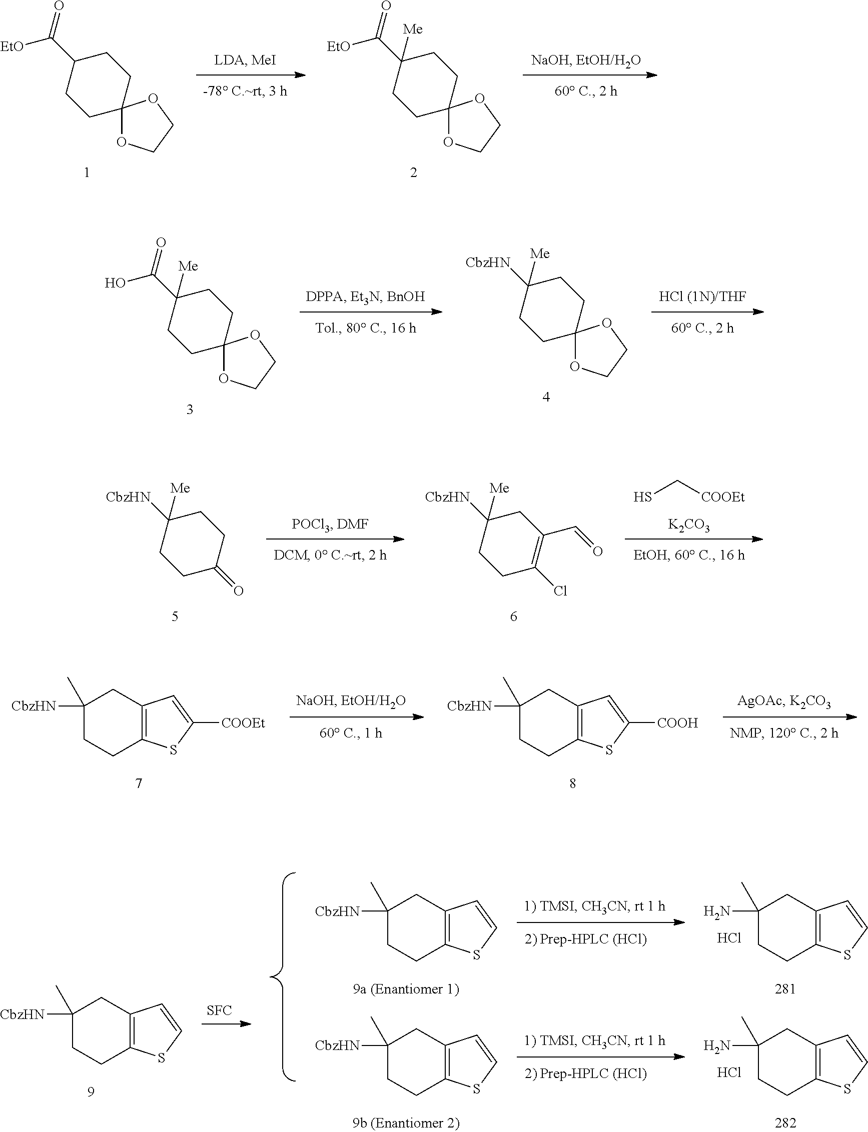

### Procedure for preparation of ethyl 8-methyl-1,4-dioxaspiro[4.5]decane-8-carboxylate (2)

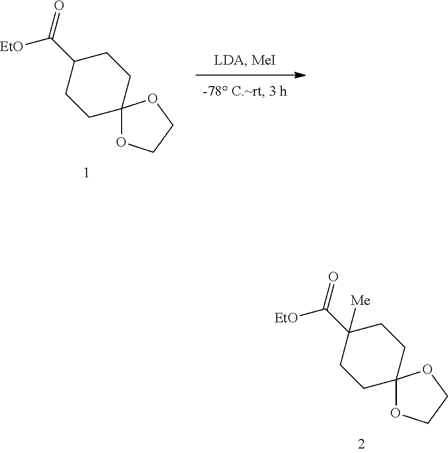 [SMILES: CCOC(=O)C1CCC2(CC1)OCCO2 ; "LDA, MeI" ; CCOC(=O)C1(C)CCC2(CC1)OCCO2]

To a solution of LDA (2 M, 140.02 mL, 1.2 eq) in THF (300 mL) was added a solution of ethyl 1,4-dioxaspiro[4.5]decane-8-carboxylate (50 g, 233.36 mmol, 1 eq) in THF (50 mL) dropwise at −78° C. under N₂. After stirring for 1 h a solution of MeI (43.06 g, 303.37 mmol, 18.89 mL, 1.3 eq) in THF (50 mL) was added dropwise under N₂. The mixture was stirred at −78° C. for 1 h, then was warmed to 0° C. and stirred for 1 hr more. The reaction mixture was then quenched by addition of aq. sat.NH₄Cl 1000 mL at 0° C., and then the mixture was extracted with EtOAc (200 mL*2). The combined organic layers were washed with brine (200 mL*2), dried over Na₂SO₄, filtered and concentrated under reduced pressure to give the crude product ethyl 8-methyl-1,4-dioxaspiro[4.5]decane-8-carboxylate (2) (60 g, crude) as a yellow oil, that was used without further purification. ¹H NMR (400 MHz, METHANOL-d4) δ=4.15 (q, J=7.1 Hz, 2H), 3.93-3.89 (m, 4H), 2.13-2.05 (m, 2H), 1.62 (s, 2H), 1.59-1.47 (m, 4H), 1.28-1.24 (m, 3H), 1.17 (s, 3H)

### Procedure for preparation of 8-methyl-1,4-dioxaspiro[4.5]decane-8-carboxylic acid (3)

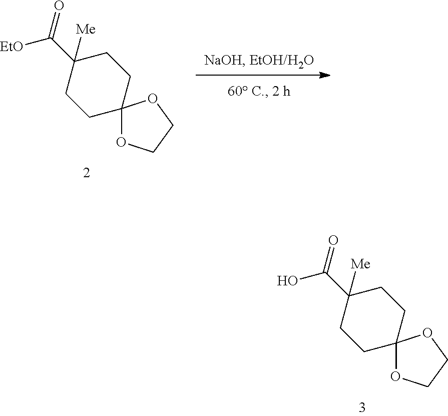 [SMILES: CCOC(=O)C1(C)CCC2(CC1)OCCO2 ; "NaOH, EtOH/H2O" ; CC1(C(=O)O)CCC2(CC1)OCCO2]

To a solution of ethyl 8-methyl-1,4-dioxaspiro[4.5]decane-8-carboxylate (2) (20 g, 87.61 mmol, 1 eq) in EtOH (150 mL) was added NaOH (10.51 g, 262.83 mmol, 3 eq) in H₂O (50 mL). The mixture was stirred at 60° C. for 2 hr. The reaction mixture cooled to room temperature, it was acidified with aq. 1 N HCl to pH=5-6, and then extracted with EtOAc (100 mL*3). The combined organic layers were washed with brine (100 mL*2), dried over Na₂SO₄, filtered and concentrated under reduced pressure to give a residue. The crude product 8-methyl-1,4-dioxaspiro[4.5]decane-8-carboxylic acid (3) (16 g, crude) was obtained as a yellow oil, and was used into the next step without further purification. ¹H NMR (400 MHz, CHLOROFORM-d) δ=3.95 (s, 4H), 2.14 (br dd, J=4.9, 13.7 Hz, 2H), 1.72-1.64 (m, 4H), 1.59-1.50 (m, 2H), 1.27 (s, 3H)

### Procedure for preparation of benzyl (8-methyl-1,4-dioxaspiro[4.5]decan-8-yl)carbamate (4)

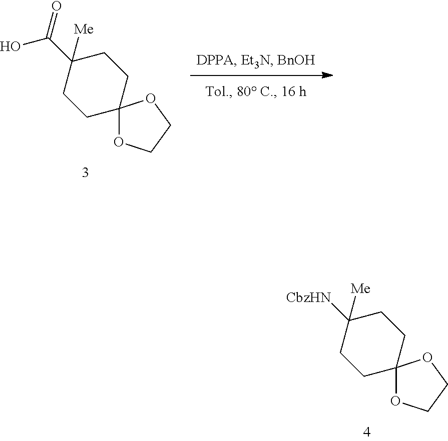

To a solution of 8-methyl-1,4-dioxaspiro[4.5]decane-8-carboxylic acid (3) (14 g, 69.92 mmol, 1 eq) in Tol. (200 mL) was added DPPA (23.09 g, 83.90 mmol, 18.11 mL, 1.2 eq), TEA (8.49 g, 83.90 mmol, 11.68 mL, 1.2 eq) and BnOH (22.68 g, 209.76 mmol, 21.73 mL, 3 eq). The mixture was degassed and purged with N₂ for 3 times, and then stirred at 80° C. for 16 hr under N₂ atmosphere. The reaction mixture was then cooled and poured into 300 mL of H₂O, and then extracted with EtOAc (50 mL*3). The combined organic layers were washed with brine (100 mL*2), dried over Na₂SO₄, filtered and concentrated under reduced pressure to give a residue. The residue was purified by flash silica gel chromatography (ISCO®; 120 g SepaFlash® Silica Flash Column, eluent 0~15% Ethyl acetate/Petroleum ether gradient @80 mL/min). Compound benzyl N-(8-methyl-1,4-dioxaspiro[4.5]decan-8-yl)carbamate (4) (28 g, 22.01 mmol, 31.47% yield) was obtained as a yellow oil. LC-MS: m/z [M+Na]⁺=328.1

### Procedure for preparation of benzyl (1-methyl-4-oxocyclohexyl)carbamate (5)

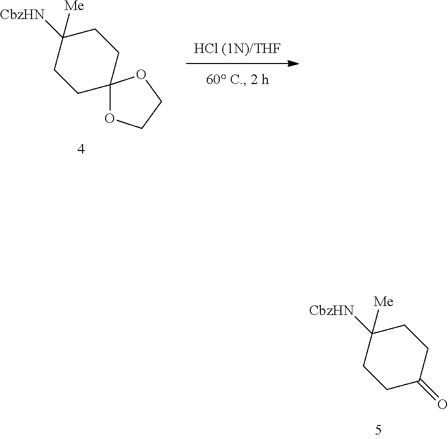 [SMILES: CC1(NC(=O)OCc2ccccc2)CCC2(CC1)OCCO2 ; "HCl (1N)/THF" ; CC1(NC(=O)OCc2ccccc2)CCC(=O)CC1]

To a solution of benzyl N-(8-methyl-1,4-dioxaspiro[4.5]decan-8-yl)carbamate (4) (30 g, 98.24 mmol, 1 eq) in THF (400 mL) was added HCl (2 M, 250.00 mL, 5.09 eq). The mixture was stirred at 60° C. for 2 hr then cooled and concentrated to remove THF. The aqueous layer was extracted with EtOAc (200 mL*3). The combined organic layers were washed with aq. sat. NaHCO₃ (200 mL), and brine (200 mL), dried over Na₂SO₄, filtered and concentrated under reduced pressure to give a residue. The residue was purified with flash silica gel chromatography (ISCO®; 120 g SepaFlash® Silica Flash Column, eluent 0-25% Ethyl acetate/Petroleum ether gradient @100 mL/min). Compound benzyl N-(1-methyl-4-oxo-cyclohexyl)carbamate (5) (9.5 g, 22.90 mmol, 23.31% yield) was obtained as a light yellow solid. ¹H NMR (400 MHz, CHLOROFORM-d) δ=7.41-7.33 (m, 5H), 5.09 (s, 2H), 4.79 (br s, 1H), 2.49-2.37 (m, 4H), 2.33-2.27 (m, 2H), 1.87-1.79 (m, 2H), 1.46 (s, 3H)

### Procedure for preparation of benzyl (4-chloro-3-formyl-1-methylcyclohex-3-en-1-yl)carbamate (6)

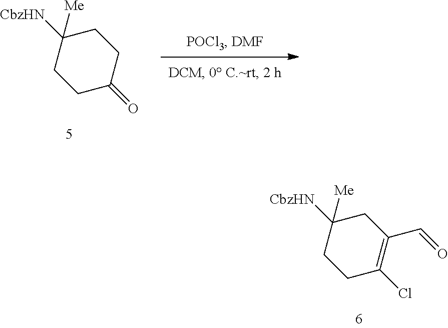

To a solution of DMF (15.94 g, 218.13 mmol, 16.78 mL, 6 eq) in DCM (200 mL) was added POCl₃ (27.87 g, 181.77 mmol, 16.94 mL, 5 eq) at 0° C. The mixture was warmed up to room temperature and stirred for 1 h. Then followed dropwise addition of benzyl N-(1-methyl-4-oxo-cyclohexyl)carbamate (5) (9.5 g, 36.35 mmol, 1 eq) in DCM (50 mL) at 0° C. The resulting mixture was warmed up and stirred at 20° C. for 2 h and then quenched by addition of aq. sat. NaHCO₃ (500 mL) at 0° C. Followed extraction with DCM (200 mL*3). The combined organic layers were washed with saturated aq.NaCl (200 mL*2), dried over anhydrous Na₂SO₄, filtered and concentrated under reduced pressure to give a residue. The residue was purified by flash silica gel chromatography (ISCO®; 80 g SepaFlash® Silica Flash Column, eluent 0~13% Ethyl acetate/Petroleum ether gradient @80 mL/min). Compound benzyl N-(4-chloro-3-formyl-1-methyl-cyclohex-3-en-1-yl)carbamate (6) (7 g, 17.74 mmol, 48.80% yield) was obtained as a white solid. LC-MS: m/z [M+H]⁺=308.0 Procedure for the preparation of ethyl 5-(((benzyloxy)carbonyl)amino)-5-methyl-4,5,6,7-tetrahydrobenzo[b]thiophene-2-carboxylate (7)

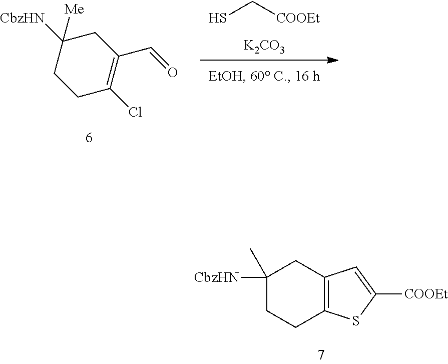

To a solution of benzyl N-(4-chloro-3-formyl-1-methyl-cyclohex-3-en-1-yl)carbamate (6) (7 g, 22.74 mmol, 1 eq) and K₂CO₃ (9.43 g, 68.23 mmol, 3 eq) in EtOH (100 mL) was added ethyl 2-sulfanylacetate (2.94 g, 24.47 mmol, 2.68 mL, 1.08 eq). The mixture was stirred at 60° C. for 16 hr. The reaction mixture was then cooled and concentrated under reduced pressure to remove EtOH. The residue was diluted with H₂O (100 mL) and extracted with EtOAc (30 mL*3). The combined organic layers were washed with saturated NaCl (50 mL*2), dried over anhydrous Na₂SO₄, filtered and concentrated under reduced pressure to give a residue. The residue was purified by flash silica gel chromatography (ISCO®; 80 g SepaFlash® Silica Flash Column, eluent 0~10% Ethyl acetate/Petroleum ether gradient @60 mL/min). Compound ethyl 5-(benzyloxycarbonylamino)-5-methyl-6,7-dihydro-4H-benzothiophene-2-carboxylate (7) (6 g, 14.62 mmol, 64.28% yield) was obtained as a light yellow oil. ¹H NMR (400 MHz, METHANOL-d4) δ=7.43 (s, 1H), 7.35-7.23 (m, 5H), 5.01 (br s, 2H), 4.28 (q, J=7.1 Hz, 2H), 3.00 (br d, J=16.0 Hz, 1H), 2.79 (br t, J=6.2 Hz, 2H), 2.64 (br d, J=16.3 Hz, 1H), 2.51-2.42 (m, 1H), 1.81-1.74 (m, 1H), 1.41 (s, 3H), 1.33 (t, J=7.1 Hz, 3H).

### Procedure for preparation of 5-(((benzyloxy)carbonyl)amino)-5-methyl-4,5,6,7-tetrahydrobenzo[b]thiophene-2-carboxylic acid (8)

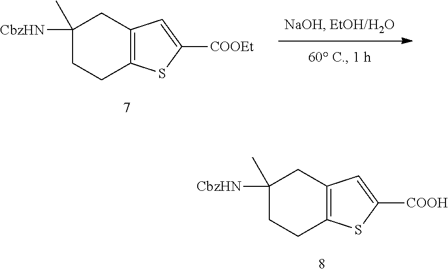 [SMILES: CCOC(=O)c1cc2c(s1)CCC(C)(NC(=O)OCc1ccccc1)C2 ; "NaOH, EtOH/H2O" ; CC1(NC(=O)OCc2ccccc2)CCc2sc(C(=O)O)cc2C1]

To a solution of ethyl 5-(benzyloxycarbonylamino)-5-methyl-6,7-dihydro-4H-benzothiophene-2-carboxylate (7) (3 g, 8.03 mmol, 1 eq) in EtOH (25 mL) was added NaOH (1 g, 25.00 mmol, 3.11 eq) in H₂O (5 mL). The mixture was stirred at 60° C. for 1 hr then cooled and concentrated under vacuum to remove EtOH. Followed dilution with H₂O (20 ml) and the resulting mixture was acidified with 1N aq. HCl to pH=3-4. A white suspension formed that was filtered. The filter cake was washed with H₂O (10 ml) and dried under high vacuum. Compound 5-(benzyloxycarbonylamino)-5-methyl-6,7-dihydro-4H-benzothiophene-2-carboxylic acid (8) (2.2 g, 5.67 mmol, 70.57% yield) was obtained as an off-white solid. ¹H NMR (400 MHz, METHANOL-d4) δ=7.38 (s, 1H), 7.36-7.24 (m, 5H), 6.78 (br s, 1H), 5.13-4.93 (m, 2H), 2.98 (br d, J=16.3 Hz, 1H), 2.79 (br t, J=6.3 Hz, 2H), 2.65 (d, J=16.3 Hz, 1H), 2.55-2.40 (m, 1H), 1.78 (td, J=7.3, 13.4 Hz, 1H), 1.41 (s, 3H)

### Procedure for preparation of benzyl (5-methyl-4,5,6,7-tetrahydrobenzo[b]thiophen-5-yl)carbamate (9)

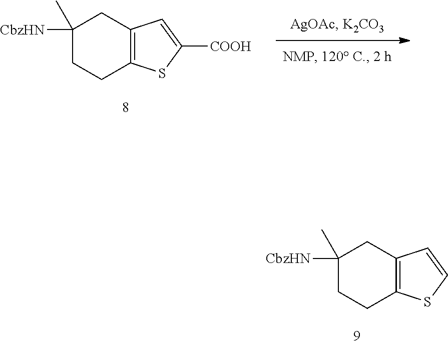

To a solution of 5-(benzyloxycarbonylamino)-5-methyl-6,7-dihydro-4H-benzothiophene-2-carboxylic acid (8) (2 g, 5.79 mmol, 1 eq) in NMP (20 mL) was added AgOAc (483.22 mg, 2.90 mmol, 0.5 eq) and K₂CO₃ (2.40 g, 17.37 mmol, 3 eq). The mixture was stirred at 120° C. for 2 hr. The reaction mixture was poured into 100 mL of H₂O, and then extracted with EtOAc (30 mL*3). The combined organic layers were washed with brine (50 mL*2), dried over Na₂SO₄, filtered and concentrated under reduced pressure to give a residue. The residue was purified by flash silica gel chromatography (ISCO®; 20 g SepaFlash® Silica Flash Column, eluent 0~10% Ethyl acetate/Petroleum ether gradient @40 mL/min). Compound benzyl N-(5-methyl-6,7-dihydro-4H-benzothiophen-5-yl)carbamate (9) (1.6 g, 4.88 mmol, 84.35% yield, 92% purity) was obtained as a colorless oil. ¹H NMR (400 MHz, CHLOROFORM-d) δ=7.40-7.29 (m, 5H), 7.09 (d, J=5.0 Hz, 1H), 6.72 (d, J=5.0 Hz, 1H), 5.06 (s, 2H), 4.73 (br s, 1H), 2.85-2.72 (m, 4H), 2.57-2.43 (m, 1H), 1.80-1.72 (m, 1H), 1.51 (s, 3H).

### Preparation of benzyl (5-methyl-4,5,6,7-tetrahydrobenzo[b]thiophen-5-yl)carbamate enantiomer 1 (9a) and enantiomer 2 (9b)

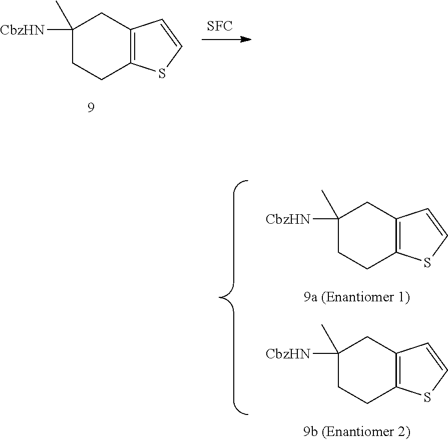

Racemic benzyl N-(5-methyl-6,7-dihydro-4H-benzothiophen-5-yl)carbamate (9) was separated to its enantiomers by SFC (condition: column: DAICEL CHIRALCEL OD (250 mm*50 mm, 10 μm); mobile phase: [CO₂-i-PrOH (0.1% NH₃H₂O)]; B %:25%, isocratic elution mode). Compound benzyl N-(5-methyl-6,7-dihydro-4H-benzothiophen-5-yl)carbamate enantiomer 1 (9a) (400 mg, 1.31 mmol, 49.50% yield) was obtained as a colorless oil. LC-MS: m/z [M+H]⁺=302.0. Compound benzyl N-(5-methyl-6,7-dihydro-4H-benzothiophen-5-yl)carbamate enantiomer 2 (9b) (384 mg, 1.26 mmol, 47.50% yield) was obtained as a colorless oil. LC-MS: m/z [M+H]⁺=302.0

### Procedure for preparation of 5-methyl-4,5,6,7-tetrahydrobenzo[b]thiophen-5-amine hydrochloride enantiomer 1 (281)

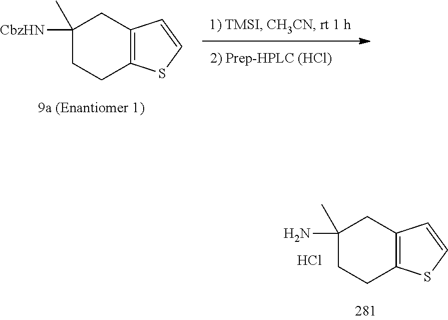 [SMILES: CC1(NC(=O)OCc2ccccc2)CCc2sccc2C1 ; Cl ; CC1(N)CCc2sccc2C1]

To a solution of benzyl N-(5-methyl-6,7-dihydro-4H-benzothiophen-5-yl)carbamate enantiomer 1 (9a) (400 mg, 1.33 mmol, 1 eq) in CH₃CN (10 mL) was added TMSI (796.65 mg, 3.98 mmol, 541.94 μL, 3 eq). The mixture was stirred at 25° C. for 1 hr. The reaction mixture was poured into 50 mL of 1 N HCl (aq.), and then aqueous phase was washed with MTBE (30 mL*3) and freeze dried to give a residue. The residue was purified by prep-HPLC (HCl condition; column: Phenomenex luna C18 150*25 mm*10 μm; mobile phase: [water(HCl)-MeOH]; gradient:1%-25% B over 10 min). Compound 5-methyl-6,7-dihydro-4H-benzothiophen-5-amine hydrochloride (281) (203.85 mg, 995.98 μmol, 75.05% yield) was obtained as a yellow solid. 1H NMR (400 MHz, METHANOL-d4) δ=7.25 (d, J=5.1 Hz, 1H), 6.79 (d, J=5.3 Hz, 1H), 2.95 (t, J=6.5 Hz, 2H), 2.91-2.81 (m, 2H), 2.11-1.99 (m, 2H), 1.42 (s, 3H). LC-MS: m/z [M+H]⁺=168.0

### Procedure for preparation of 5-methyl-4,5,6,7-tetrahydrobenzo[b]thiophen-5-amine hydrochloride enantiomer 2 (282)

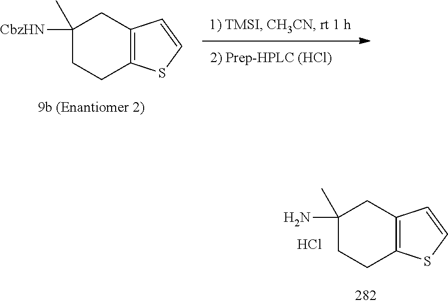 [SMILES: CC1(NC(=O)OCc2ccccc2)CCc2sccc2C1 ; Cl ; CC1(N)CCc2sccc2C1]

To a solution of benzyl N-(5-methyl-6,7-dihydro-4H-benzothiophen-5-yl)carbamate enantiomer 2 (9b) (384 mg, 1.26 mmol, 1 eq) in CH₃CN (10 mL) was added TMSI (796.65 mg, 3.98 mmol, 541.94 μL, 3 eq). The mixture was stirred at 25° C. for 1 hr and then poured into 50 mL of 1 N HCl (aq). The aqueous phase was washed with MTBE (30 mL*3), and then freeze dried to a residue that was purified by prep-HPLC (HCl condition; column: Phenomenex luna C18 150*25 mm*10 μm; mobile phase: [water(HCl)-ACN]; gradient:1%-25% B over 10 min). Compound 5-methyl-6,7-dihydro-4H-benzothiophen-5-amine enantiomer 2 (282) (136.11 mg, 668.08 μmol, 50.34% yield) was obtained as a yellow solid. ¹H NMR (400 MHz, METHANOL-d4) δ=7.25 (d, J=5.3 Hz, 1H), 6.79 (d, J=5.1 Hz, 1H), 2.95 (t, J=6.6 Hz, 2H), 2.91-2.81 (m, 2H), 2.05 (sxtd, J=6.8, 14.0 Hz, 2H), 1.42 (s, 3H). LC-MS: m/z [M+H]⁺=168.0

### Synthetic scheme for preparation of 1-(5-methyl-4,5,6,7-tetrahydrobenzo[b]thiophen-5-yl)pyrrolidine hydrochloride enantiomer 1 (283) and enantiomer 2 (284)

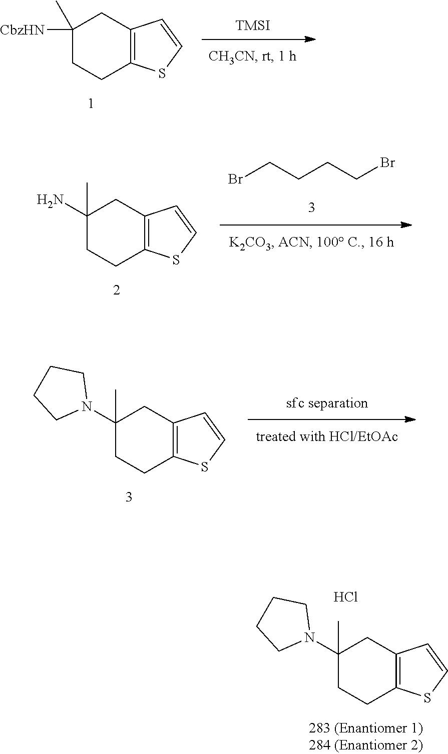

### Procedure for preparation of 5-methyl-4,5,6,7-tetrahydrobenzo[b]thiophen-5-amine (2)

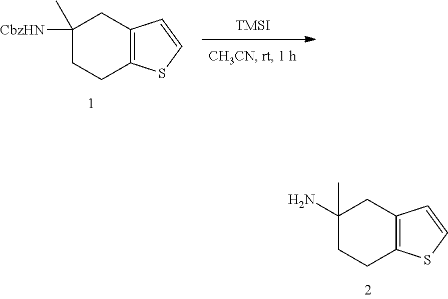 [SMILES: "TMSI" ; CC1(NC(=O)OCc2ccccc2)CCc2sccc2C1 ; CC1(N)CCc2sccc2C1]

To a solution of racemic benzyl N-(5-methyl-6,7-dihydro-4H-benzothiophen-5-yl)carbamate (1) (800 mg, 2.65 mmol, 1 eq) in CH₃CN (15 mL) was added TMSI (1.59 g, 7.96 mmol, 1.08 mL, 3 eq). The mixture was stirred at 25° C. for 1 hr and then poured into 50 mL of aq. 1 N HCl. The aqueous phase was extracted with MTBE (30 mL*3), and then freeze dried to afford 5-methyl-6,7-dihydro-4H-benzothiophen-5-amine (2) (400 mg, crude) as a colorless oil. LC-MS: m/z [M+H]⁺=168.0

### Procedure for preparation of 1-(5-methyl-4,5,6,7-tetrahydrobenzo[b]thiophen-5-yl)pyrrolidine formate salt (3)

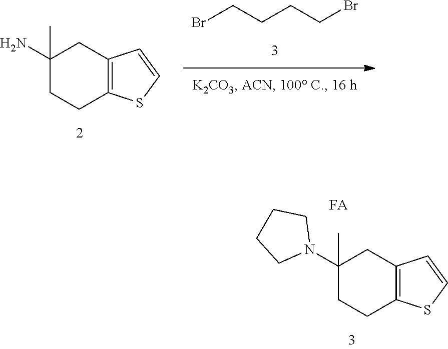

To a solution of 5-methyl-6,7-dihydro-4H-benzothiophen-5-amine (2) (150 mg, 896.75 μmol, 1 eq) and 1,4-dibromobutane (212.98 mg, 986.42 μmol, 119.05 μL, 1.1 eq) in ACN (2 mL) was added K₂CO₃ (371.81 mg, 2.69 mmol, 3 eq). The mixture was stirred at 100° C. for 16 hr then cooled filtered and concentrated under reduced pressure to give a residue which was p purified by reverse-phase HPLC (C18 30-40% ACN in water (0.1% FA condition). Compound 1-(5-methyl-6,7-dihydro-4H-benzothiophen-5-yl)pyrrolidine formate salt (3) (150 mg, 677.63 μmol, 75.56% yield) was obtained as a white solid. LC-MS: m/z [M+H]⁺=222.0

### Procedure for preparation of 1-(5-methyl-4,5,6,7-tetrahydrobenzo[b]thiophen-5-yl)pyrrolidine hydrochloride enantiomer 1 (283) and enantiomer 2 (284)

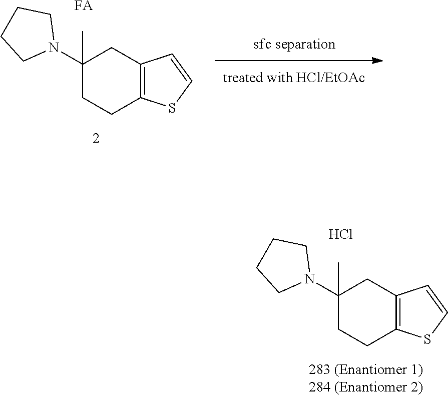

Racemic 1-(5-methyl-4,5,6,7-tetrahydrobenzo[b]thiophen-5-yl)pyrrolidine formate salt (2) (150 mg) was separated to its enantiomers by SFC (column: DAICEL CHIRALPAK AD (250 mm*50 mm, 10 μm); mobile phase: [CO₂-EtOH (0.1% NH₃H₂O)]; B %:17%, isocratic elution mode). Each enantiomer was treated with 4N HCl/EtOAc solution to provide the corresponding HCl salts that were collected, triturated with EtOAc and then dried. Compound 1-(5-methyl-6,7-dihydro-4H-benzothiophen-5-yl)pyrrolidine hydrochloride enantiomer 1 (283) (36.88 mg, 142.76 μmol, 21.07% yield) was obtained as an off-white solid. ¹H NMR (400 MHz, METHANOL-d₄) δ=7.25 (d, J=5.1 Hz, 1H), 6.80 (d, J=5.1 Hz, 1H), 3.56-3.37 (m, 4H), 3.09-2.96 (m, 3H), 2.95-2.85 (m, 1H), 2.22-2.01 (m, 6H), 1.39 (s, 3H). LC-MS: m/z [M+H]⁺=222.1. Compound 1-(5-methyl-6,7-dihydro-4H-benzothiophen-5-yl)pyrrolidine hydrochloride enantiomer 2 (284) (32.12 mg, 122.22 μmol, 18.04% yield) was obtained as an orange solid. ¹H NMR (400 MHz, METHANOL-d₄) δ=7.25 (d, J=5.1 Hz, 1H), 6.80 (d, J=5.3 Hz, 1H), 3.57-3.37 (m, 4H), 3.09-2.95 (m, 3H), 2.94-2.86 (m, 1H), 2.22-2.01 (m, 6H), 1.39 (s, 3H). LC-MS: m/z [M+H]⁺=222.1

### Synthetic scheme for preparation of N, 2,6-trimethyl-5,7-dihydrothieno[3,2-b]pyran-6-amine hydrochloride (285)

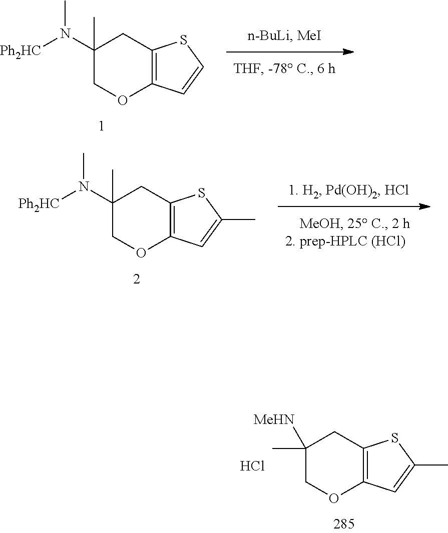

### Procedure for preparation of N-benzhydryl-N, 2,6-trimethyl-5,7-dihydrothieno[3,2-b]pyran-6-amine (2)

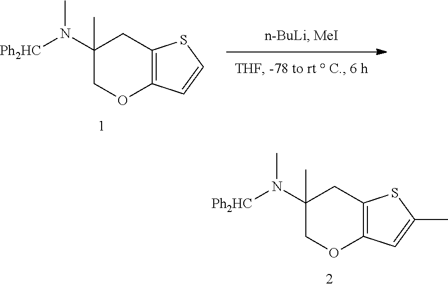

To a solution of N-benzhydryl-N, 6-dimethyl-5,7-dihydrothieno[3,2-b]pyran-6-amine (1) (800.00 mg, 2.29 mmol, 1 eq) in THF (10 mL) was added butyllithium (2.5 M, 1.37 mL, 1.5 eq) at −78° C. The mixture was stirred at −78° C. for 0.5 hr, then MeI (389.89 mg, 2.75 mmol, 171.00 μL, 1.2 eq) was added to the mixture. The mixture was allowed to warm up slowly to rt and stirred for 5.5 hr. The reaction mixture was quenched by addition aq. sat. NH₄Cl (60 mL) at 25° C. and extracted with EtOAc (40 mL*2). The combined organic layers were washed with aq. sat. NaCl (50 mL*2), dried over Na₂SO₄, filtered and concentrated under reduced pressure to give a residue. The residue was purified by prep-HPLC (neutral condition: column: Waters Xbridge 150*25 mm*5 μm; mobile phase: [water(NH₄HCO₃)-ACN]; gradient:75%-100% B over 9 min). Compound N-benzhydryl-N, 2,6-trimethyl-5,7-dihydrothieno[3,2-b]pyran-6-amine (2) (430 mg, 1.18 mmol, 51.68% yield) was obtained as a white solid. LC-MS: m/z [M+H]⁺=364.0

### Procedure for preparation of N, 2,6-trimethyl-5,7-dihydrothieno[3,2-b]pyran-6-amine hydrochloride (285)

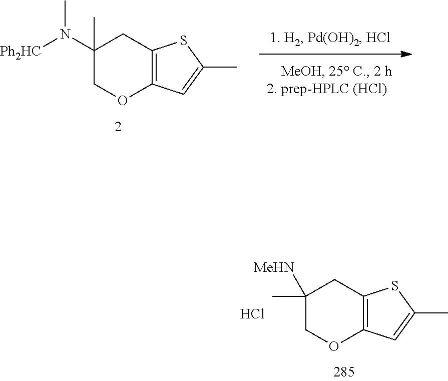

To a solution of N-benzhydryl-N, 2,6-trimethyl-5,7-dihydrothieno[3,2-b]pyran-6-amine (2) (200 mg, 550.18 μmol, 1 eq) in MeOH (5 mL) was added Pd(OH)₂ on carbon (20% wt, 50% H₂O, 69.85 mg, 248.68 μmol) and aq. HCl (0.1 mL, 12M) under N₂. The suspension was degassed under vacuum and purged with H₂ several times. The mixture was stirred under H₂ (50 psi) at 25° C. for 2 hours then filtered and concentrated under reduced pressure to remove MeOH. The residue was purified by prep-HPLC (HCl condition:column: Phenomenex luna C18 150*25 mm*10 μm; mobile phase: [water(HCl)-ACN]; gradient:1%-25% B over 10 min). Compound N, 2,6-trimethyl-5,7-dihydrothieno[3,2-b]pyran-6-amine hydrochloride (285) (52.94 mg, 226.47 μmol, 41.16% yield) was obtained as a white solid. LC-MS: m/z [M+H]⁺=198.0. ¹H NMR (400 MHz, METHANOL-d₄) δ=6.43 (s, 1H), 4.29 (dd, J=1.9, 12.3 Hz, 1H), 4.00 (d, J=12.3 Hz, 1H), 3.07-2.93 (m, 2H), 2.72 (s, 3H), 2.40 (s, 3H), 1.45 (s, 3H)

### Synthetic scheme for synthesis of N-methyl-5-(trifluoromethyl)-3,4-dihydro-2H-thieno[2,3-b]pyran-3-amine hydrochloride racemate (288) enantiomer 1 (289) and enantiomer 2 (290)

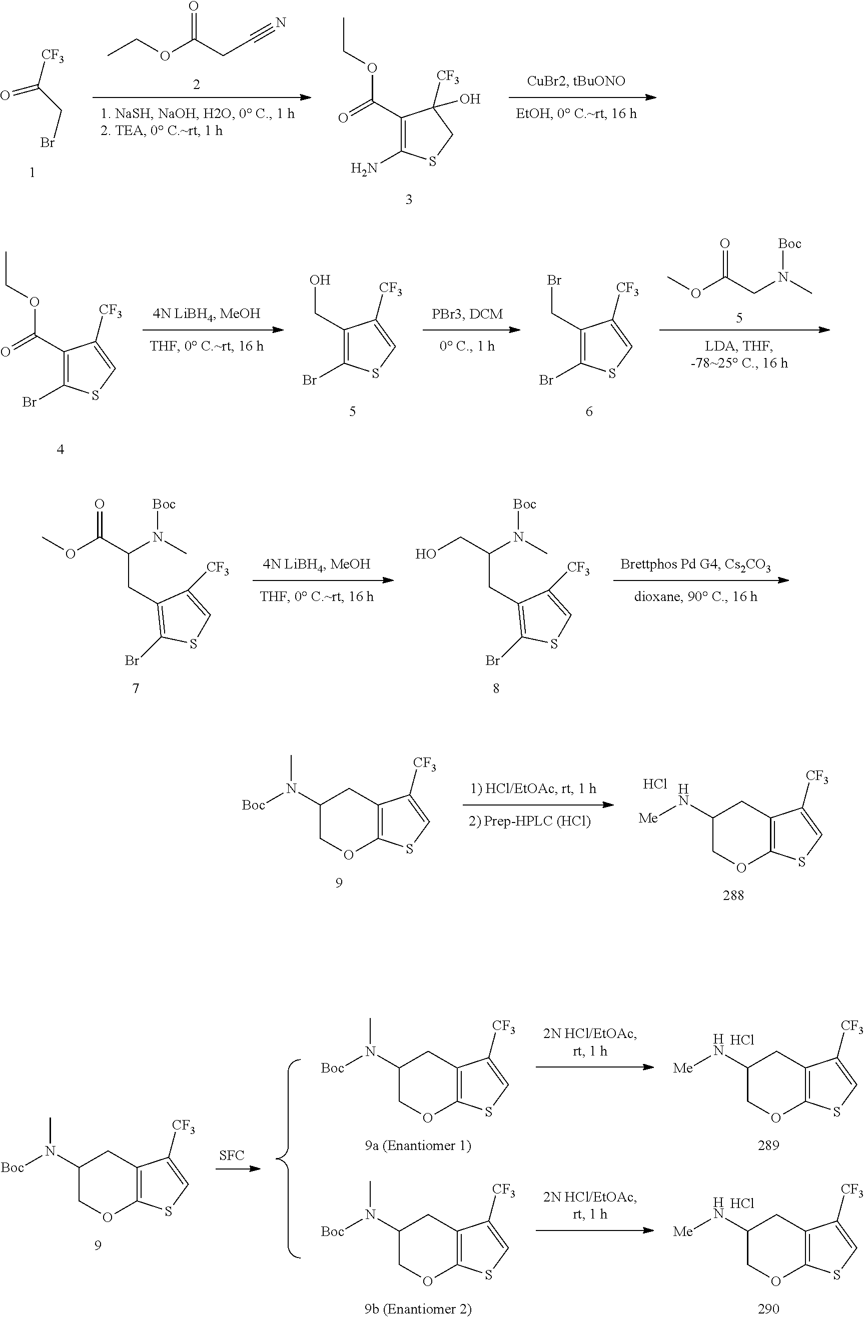

### Procedure for preparation of ethyl 5-amino-3-hydroxy-3-(trifluoromethyl)-2H-thiophene-4-carboxylate (3)

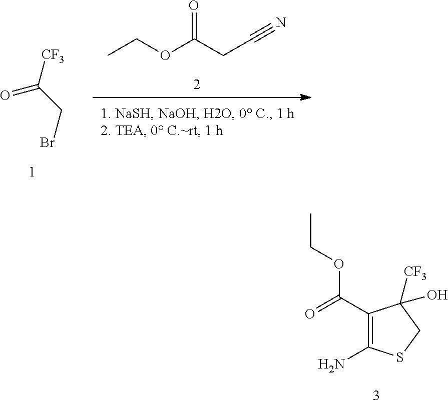

To a solution of sulfanylsodium; hydrate (38.80 g, 366.59 mmol, 1.4 eq) in H₂O (150 mL) at 0° C. was added NaOH (1 M, 150 mL, 5.73e-1 eq) dropwise, followed by a solution of 3-bromo-1,1,1-trifluoro-propan-2-one (50 g, 261.85 mmol, 27.19 mL, 1 eq). The mixture was stirred at 0° C. for 1 h. To the mixture was then added ethyl 2-cyanoacetate (29.62 g, 261.85 mmol, 27.92 mL, 1 eq) at 0° C. and TEA (26.50 g, 261.85 mmol, 36.45 mL, 1 eq). The mixture was stirred at 0° C. for 1 h. The solid formed collected by filtration and was washed with 100 mL of cold water. Compound ethyl 5-amino-3-hydroxy-3-(trifluoromethyl)-2H-thiophene-4-carboxylate (3) (19 g, 73.86 mmol, 28.21% yield) was obtained as a white solid. ¹H NMR (400 MHz, CHLOROFORM-d) δ=7.05-5.48 (m, 1H), 5.42-5.02 (m, 1H), 4.39-4.19 (m, 2H), 3.61-3.51 (m, 1H), 3.48-3.40 (m, 1H), 1.33 (t, J=7.1 Hz, 3H)

### Procedure for preparation of ethyl 2-bromo-4-(trifluoromethyl)thiophene-3-carboxylate (4)

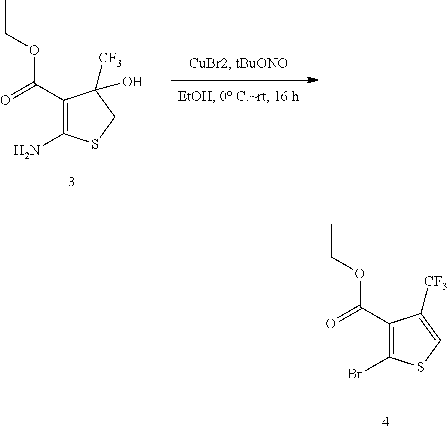 [SMILES: CCOC(=O)C1=C(N)SCC1(O)C(F)(F)F ; "CuBr2, tBuONO" ; CCOC(=O)c1c(C(F)(F)F)csc1Br]

To a solution of ethyl 5-amino-3-hydroxy-3-(trifluoromethyl)-2H-thiophene-4-carboxylate (3) (19 g, 73.86 mmol, 1 eq) in EtOH (200 mL) was added CuBr₂ (19.80 g, 88.64 mmol, 4.15 mL, 1.2 eq) and tert-butyl nitrite (11.43 g, 110.80 mmol, 13.18 mL, 1.5 eq) at 0° C. The mixture was slowly warmed to 25° C. and stirred for 16 hr. The solvent was then removed in vacuo and the residue was purified by flash silica gel chromatography (ISCO®; 120 g SepaFlash® Silica Flash Column, eluent 0~9% Ethyl acetate/Petroleum ether gradient @40 mL/min). Compound ethyl 2-bromo-4-(trifluoromethyl)thiophene-3-carboxylate (4) (9.2 g, 30.35 mmol, 41.09% yield) was obtained as a colorless oil. ¹H NMR (400 MHz, DMSO-d6) δ=8.42-8.38 (m, 1H), 4.36-4.22 (m, 2H), 1.34-1.23 (m, 3H)

### Procedure for preparation of [2-bromo-4-(trifluoromethyl)-3-thienyl]methanol (5)

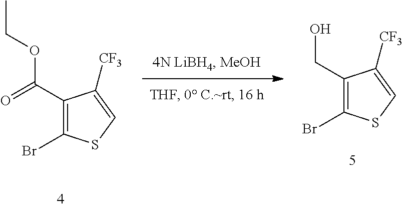

To a solution of ethyl 2-bromo-4-(trifluoromethyl)thiophene-3-carboxylate (3) (9 g, 29.69 mmol, 1 eq) in THF (90 mL) was added LiBH₄ (4 M, 37.12 mL, 5 eq) and then MeOH (5 mL) at 0° C. The mixture temperature was allowed to naturally rise to 25° C. and stirred for 16 hr. The reaction mixture was then quenched with aq. sat. NH₄C₁ (100 mL) and extracted with EtOAc (50 mL*2). The combined organic layers were washed with sat. NaCl (50 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~19% Ethyl acetate/Petroleum ether gradient @40 mL/min). Compound [2-bromo-4-(trifluoromethyl)-3-thienyl]methanol (5) (4.3 g, 16.47 mmol, 55.47% yield) was obtained as a colorless oil. ¹H NMR (400 MHz, CHLOROFORM-d) 6=7.72 (d, J=0.6 Hz, 1H), 4.71 (d, J=6.1 Hz, 2H), 1.80 (br t, J=6.3 Hz, 1H).

### Procedure for preparation of 2-bromo-3-(bromomethyl)-4-(trifluoromethyl)thiophene (6)

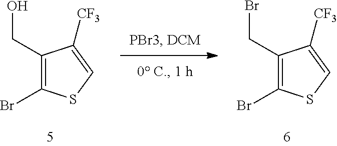 [SMILES: OCc1c(C(F)(F)F)csc1Br ; "PBr3, DCM" ; FC(F)(F)c1csc(Br)c1CBr]

To a solution of [2-bromo-4-(trifluoromethyl)-3-thienyl]methanol (5) (4.3 g, 16.47 mmol, 1 eq) in DCM (40 mL) was added PBr₃ (6.69 g, 24.71 mmol, 1.5 eq). The mixture was stirred at 0° C. for 1 hr and then quenched with aq. sat.NaHCO₃ (50 mL) and extracted with DCM (50 mL*2). The combined organic layers were washed with aq. sat.NaCl (50 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~5% Ethyl acetate/Petroleum ether gradient @40 mL/min). Compound 2-bromo-3-(bromomethyl)-4-(trifluoromethyl)thiophene (6) (2 g, 6.17 mmol, 37.48% yield) was obtained as a colorless oil. ¹H NMR (400 MHz, CHLOROFORM-d) δ=7.72 (d, J=0.6 Hz, 1H), 4.50 (s, 2H)

### Procedure for preparation of methyl 3-[2-bromo-4-(trifluoromethyl)-3-thienyl]-2-[tert-butoxycarbonyl(methyl)amino]propanoate (7)

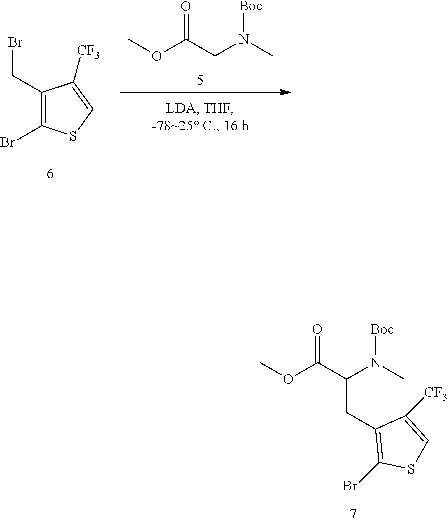

To a solution of LDA (2 M in THF, 4.63 mL, 1.5 eq) in THF (20 mL) was added dropwise methyl 2-[tert-butoxycarbonyl(methyl)amino]acetate (1.88 g, 9.26 mmol, 1.5 eq) at −78° C. under N₂. The mixture was stirred at this temperature for 1 hr, and then 2-bromo-3-(bromomethyl)-4-(trifluoromethyl)thiophene (6) (2 g, 6.17 mmol, 1 eq) was added at −78° C. The resulting mixture was allowed to slowly warm up to 25° C. and stirred for 15 hr and then quenched with aq. sat.NH₄Cl (50 mL) and extracted with EtOAc (50 mL*2). The combined organic layers were washed with aq. sat.NaCl (50 mL), dried over anhydrous Na₂SO₄, filtered and concentrated under reduced pressure to give a residue. The residue was purified by flash silica gel chromatography (ISCO®; 20 g SepaFlash® Silica Flash Column, eluent 0~13% Ethyl acetate/Petroleum ether gradient @40 mL/min). Compound methyl 3-[2-bromo-4-(trifluoromethyl)-3-thienyl]-2-[tert-butoxycarbonyl(methyl)amino]propanoate (7) (1.4 g, 3.14 mmol, 50.81% yield) was obtained as a colorless oil. ¹H NMR (400 MHz, CHLOROFORM-d) δ=7.77-7.66 (m, 1H), 4.58-4.42 (m, 1H), 3.81-3.74 (m, 3H), 3.41-3.17 (m, 2H), 2.81-2.71 (m, 3H), 1.42-1.32 (m, 9H).

### Procedure for preparation of tert-butyl N-[1-[[2-bromo-4-(trifluoromethyl)-3-thienyl]methyl]-2-hydroxy-ethyl]-N-methyl-carbamate (8)

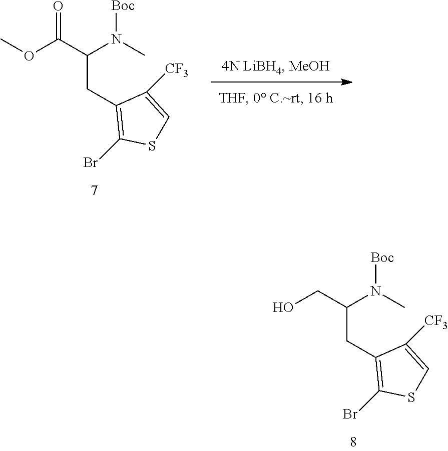

To a solution of methyl 3-[2-bromo-4-(trifluoromethyl)-3-thienyl]-2-[tert-butoxycarbonyl(methyl)amino]propanoate (7) (1.4 g, 3.14 mmol, 1 eq) in THF (20 mL) was added LiBH₄ (4 M, 3.92 mL, 5 eq) at 0° cand then MeOH (2 mL). The temperature of the mixture was allowed to naturally rise to 25° C. and stirred for 16 hr under N₂. The reaction mixture was then quenched with aq. sat.NH₄Cl 10 mL and extracted with EtOAc (30 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~30% Ethyl acetate/Petroleum ether gradient @40 mL/min). Compound tert-butyl N-[1-[[2-bromo-4-(trifluoromethyl)-3-thienyl]methyl]-2-hydroxy-ethyl]-N-methyl-carbamate (8) (1 g, 2.39 mmol, 76.21% yield) was obtained as a colorless oil. ¹H NMR (400 MHz, CHLOROFORM-d) δ=7.71 (s, 1H), 4.47-4.19 (m, 1H), 3.85-3.58 (m, 2H), 3.16-2.63 (m, 6H), 1.49-1.31 (m, 9H).

### Procedure for preparation of tert-butyl N-methyl-N-[5-(trifluoromethyl)-3,4-dihydro-2H-thieno[2,3-b]pyran-3-yl]carbamate (9)

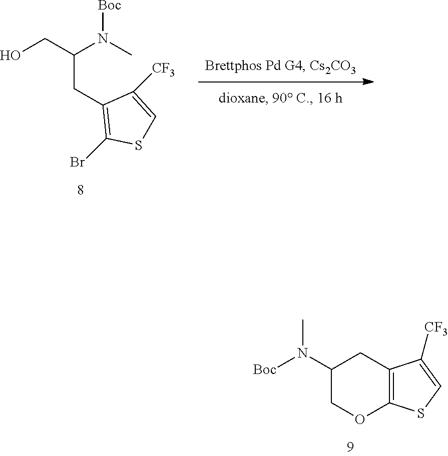 [SMILES: CN(C(=O)OC(C)(C)C)C(CO)Cc1c(C(F)(F)F)csc1Br ; "Brettphos Pd G4, Cs2CO3" ; CN(C(=O)OC(C)(C)C)C1COc2scc(C(F)(F)F)c2C1]

A mixture of tert-butyl N-[1-[[2-bromo-4-(trifluoromethyl)-3-thienyl]methyl]-2-hydroxy-ethyl]-N-methyl-carbamate (8) (1 g, 2.39 mmol, 1 eq), Brettphos Pd G4 (220.08 mg, 239.08 μmol, 0.1 eq) and Cs₂CO₃ (2.34 g, 7.17 mmol, 3 eq) in dioxane (100 mL) was degassed and purged with N₂ for 3 times. The mixture was stirred at 90° C. for 16 hr under N₂ atmosphere, cooled and filtered through a pad of Celite. The filter cake was washed with EtOAc (50 ml*3). The combined filtrate was concentrated to dryness. The residue was purified by flash silica gel chromatography (ISCO®; 40 g SepaFlash® Silica Flash Column, eluent 0~7% Ethyl acetate/Petroleum ether gradient @40 mL/min). Compound tert-butyl N-methyl-N-[5-(trifluoromethyl)-3,4-dihydro-2H-thieno[2,3-b]pyran-3-yl]carbamate (9) (400 mg, 1.19 mmol, 49.59% yield) was obtained as a colorless oil. ¹H NMR (400 MHz, CHLOROFORM-d) δ=6.94 (d, J=1.1 Hz, 1H), 4.66-4.40 (m, 1H), 4.28-4.12 (m, 2H), 2.98-2.76 (m, 5H), 1.49 (s, 9H).

### Procedure for preparation of racemic N-methyl-5-(trifluoromethyl)-3,4-dihydro-2H-thieno[2,3-b]pyran-3-amine hydrochloride (288)

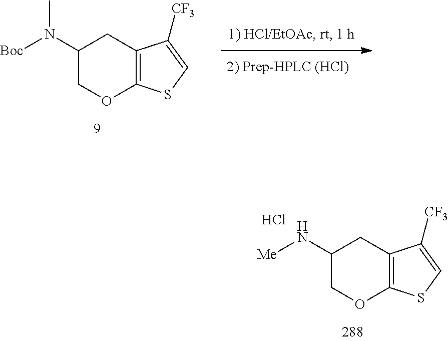

To a solution of tert-butyl N-methyl-N-[5-(trifluoromethyl)-3,4-dihydro-2H-thieno[2,3-b]pyran-3-yl]carbamate (9) (100 mg, 296.42 μmol, 1 eq) in EtOAc (2 mL) was added HCl/EtOAc (2 M, 5 mL, 33.74 eq). The mixture was stirred at 25° C. for 2 hr. and then concentrated. The residue was purified by prep-HPLC (column: Phenomenex luna C18 150*25 mm*10 μm; mobile phase: [water(HCl)-ACN]; gradient:1%-25% B over 10 min). Compound N-methyl-5-(trifluoromethyl)-3,4-dihydro-2H-thieno[2,3-b]pyran-3-amine hydrochloride (288) (30.16 mg, 110.19 μmol, 37.17% yield) was obtained as a white solid. ¹H NMR (400 MHz, METHANOL-d4) δ=7.33 (s, 1H), 4.67 (br dd, J=1.7, 12.3 Hz, 1H), 4.37 (d, J=12.4 Hz, 1H), 3.87 (br s, 1H), 3.25 (dd, J=5.8, 17.6 Hz, 1H), 2.95 (br d, J=17.5 Hz, 1H), 2.83 (s, 3H). LCMS: m/z [M+H]⁺=238.0.

### Procedure for preparation of tert-butyl N-methyl-N-[5-(trifluoromethyl)-3,4-dihydro-2H-thieno[2,3-b]pyran-3-yl]carbamate enantiomer 1 (9a) and enantiomer 2 (9b)

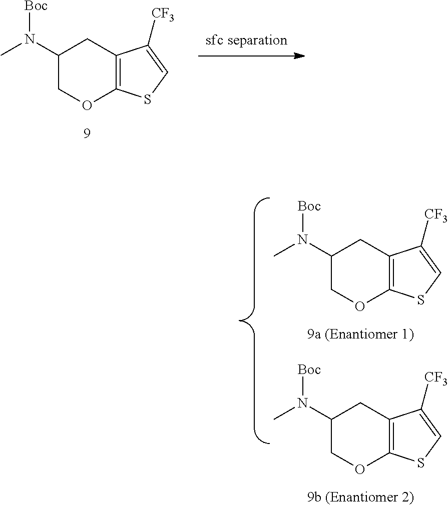 [SMILES: CN(C(=O)OC(C)(C)C)C1COc2scc(C(F)(F)F)c2C1 ; "sfc separation" ; CN(C(=O)OC(C)(C)C)C1COc2scc(C(F)(F)F)c2C1 ; CN(C(=O)OC(C)(C)C)C1COc2scc(C(F)(F)F)c2C1]

Racemic tert-butyl N-methyl-N-[5-(trifluoromethyl)-3,4-dihydro-2H-thieno[2,3-b]pyran-3-yl]carbamate (9) was separated to its enantiomers by SFC (column: DAICEL CHIRALPAK IG (250 mm*50 mm, 10 μm); mobile phase: [CO₂—Heptane-EtOH (0.1% NH₃H₂O)]; B %:10%, isocratic elution mode). tert-Butyl N-methyl-N-[5-(trifluoromethyl)-3,4-dihydro-2H-thieno[2,3-b]pyran-3-yl]carbamate enantiomer 1 (9a) (RT=0.477 min, 100 mg, 296.42 μmol, 50.00% yield) and enantiomer 2 (9b) (RT=0.583 min, 70 mg, 207.50 μmol, 35.00% yield), both were isolated as colorless oils.

### Procedure for preparation of N-methyl-5-(trifluoromethyl)-3,4-dihydro-2H-thieno[2,3-b]pyran-3-amine hydrochloride enantiomer 1 (289)

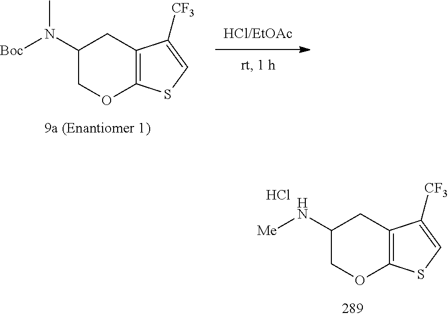 [SMILES: CN(C(=O)OC(C)(C)C)C1COc2scc(C(F)(F)F)c2C1 ; Cl ; CNC1COc2scc(C(F)(F)F)c2C1]

To a solution of tert-butyl N-methyl-N-[5-(trifluoromethyl)-3,4-dihydro-2H-thieno[2,3-b]pyran-3-yl]carbamate enantiomer 1 (9a) (100 mg, 296.42 μmol, 1 eq) in EtOAc (2 mL) was added HCl/EtOAc (2 M, 2 mL, 13.49 eq). The mixture was stirred at rt for 1 hr and then filtered. The cake was washed with EtOAc (2 mL) dried and collected to afford the product. Compound N-methyl-5-(trifluoromethyl)-3,4-dihydro-2H-thieno[2,3-b]pyran-3-amine hydrochloride enantiomer 1 (289) (55.89 mg, 204.20 μmol, 68.89% yield) was obtained as a white solid. ¹H NMR (400 MHz, METHANOL-d4) δ=7.33 (s, 1H), 4.67 (br dd, J=1.7, 12.3 Hz, 1H), 4.37 (d, J=12.4 Hz, 1H), 3.87 (br s, 1H), 3.25 (dd, J=5.8, 17.6 Hz, 1H), 2.95 (br d, J=17.5 Hz, 1H), 2.83 (s, 3H). LCMS: m/z [M+H]⁺=238.0.

### Procedure of preparation of N-methyl-5-(trifluoromethyl)-3,4-dihydro-2H-thieno[2,3-b]pyran-3-amine hydrochloride enantiomer 2 (290)

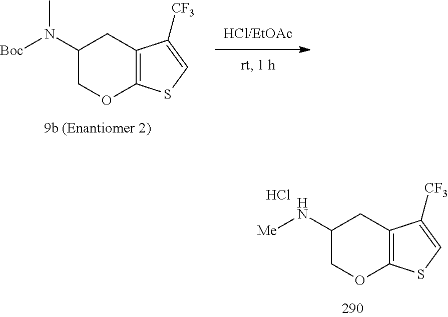 [SMILES: CN(C(=O)OC(C)(C)C)C1COc2scc(C(F)(F)F)c2C1 ; Cl ; CNC1COc2scc(C(F)(F)F)c2C1]

To a solution of tert-butyl N-methyl-N-[5-(trifluoromethyl)-3,4-dihydro-2H-thieno[2,3-b]pyran-3-yl]carbamate enantiomer 2 (9b) (70 mg, 207.50 μmol, 1 eq) in EtOAc (2 mL) was added HCl/EtOAc (2 M, 2 mL, 19.28 eq). The mixture was stirred at rt for 1 hr. The reaction mixture was then filtered and the cake was washed with EtOAc (2 mL), then dried and collected to afford the product. Compound N-methyl-5-(trifluoromethyl)-3,4-dihydro-2H-thieno[2,3-b]pyran-3-amine hydrochloride enantiomer 2 (290) (45.87 mg, 167.59 μmol, 80.77% yield) was obtained as a white solid. ¹H NMR (400 MHz, METHANOL-d4) δ=7.33 (s, 1H), 4.67 (br dd, J=1.7, 12.3 Hz, 1H), 4.37 (d, J=12.4 Hz, 1H), 3.87 (br s, 1H), 3.25 (dd, J=5.8, 17.6 Hz, 1H), 2.95 (br d, J=17.5 Hz, 1H), 2.83 (s, 3H). LCMS: m/z [M+H]⁺=238.0.

### Synthetic scheme for preparation of 5-ethyl-N-methyl-6,7-dihydro-4H-benzothiophen-5-amine hydrochloride enantiomer 1 (291) and enantiomer 2 (292)

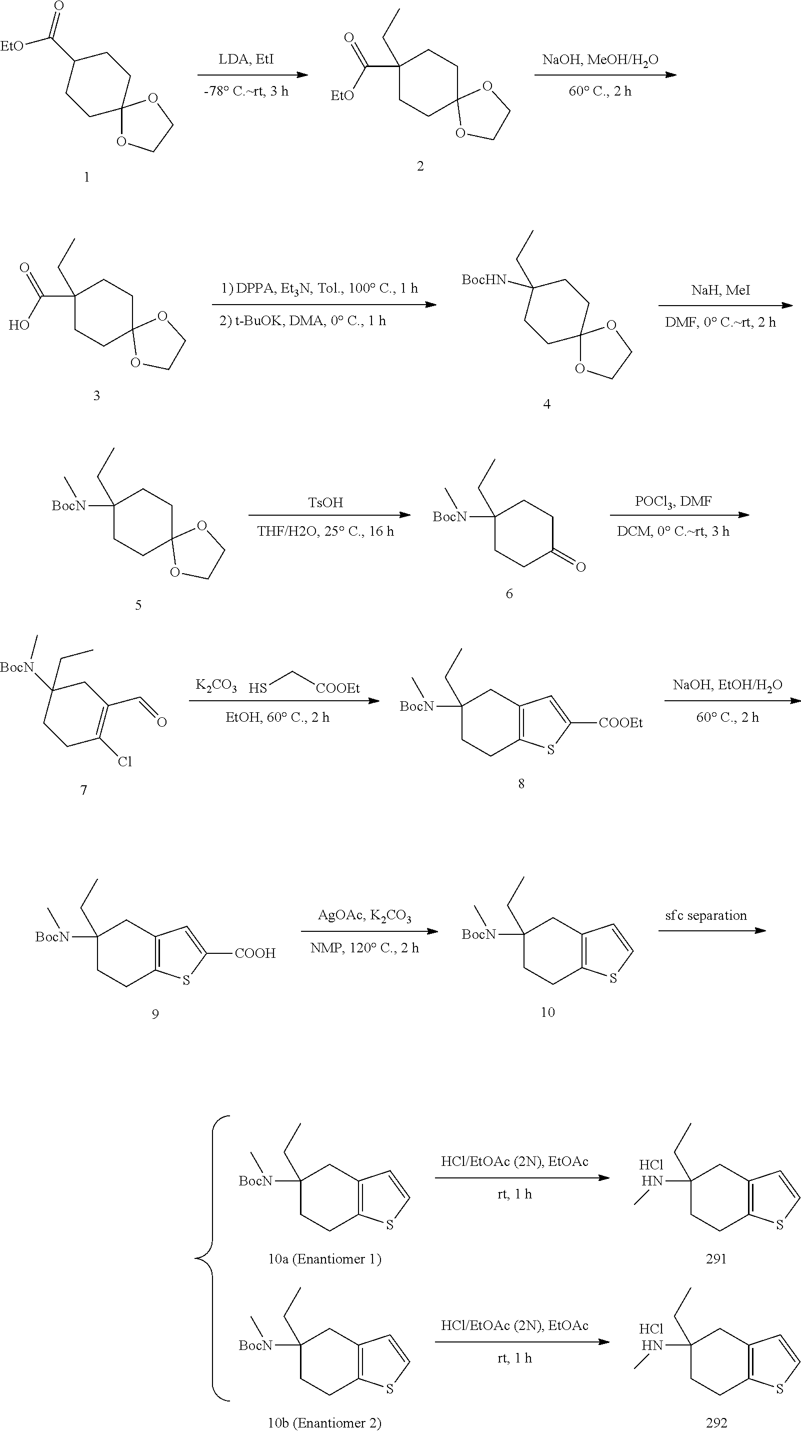

### Procedure for preparation of ethyl 8-ethyl-1,4-dioxaspiro[4.5]decane-8-carboxylate (2)

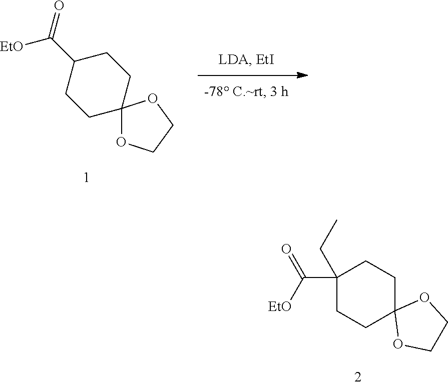 [SMILES: CCOC(=O)C1CCC2(CC1)OCCO2 ; "LDA, EtI" ; CCOC(=O)C1(CC)CCC2(CC1)OCCO2]

To a solution of LDA (2 M in THF, 56 mL, 1.2 eq) in THF (100 mL) was added ethyl 1,4-dioxaspiro[4.5]decane-8-carboxylate (1) (20 g, 93.35 mmol, 1 eq) in THF (50 mL) at −78° C. dropwise under N₂. After 1 hour a solution of EtI (17.47 g, 112 mmol, 8.96 mL, 1.2 eq) in THF (10 mL) was added at −78° C. The mixture was stirred at −78° C. for 2 hr then quenched with sat. aq. NH₄Cl (200 mL) at 0° C., diluted with H₂O (100 mL) and extracted with EtOAc (200 mL*2). The combined organic layers were washed with brine, dried over anhydrous Na₂SO₄, filtered and concentrated under reduced pressure to give the product. Compound ethyl 8-ethyl-1,4-dioxaspiro[4.5]decane-8-carboxylate (2) (22.6 g, crude) was obtained as a yellow oil. ¹H NMR (400 MHz, CHLOROFORM-d) δ=4.15 (q, J=7.0 Hz, 2H), 3.93 (s, 4H), 2.15 (br d, J=13.0 Hz, 2H), 1.66-1.52 (m, 6H), 1.51-1.41 (m, 2H), 1.26 (t, J=7.1 Hz, 3H), 0.82 (t, J=7.5 Hz, 3H).

### Procedure for preparation of 8-ethyl-1,4-dioxaspiro[4.5]decane-8-carboxylic acid (3)

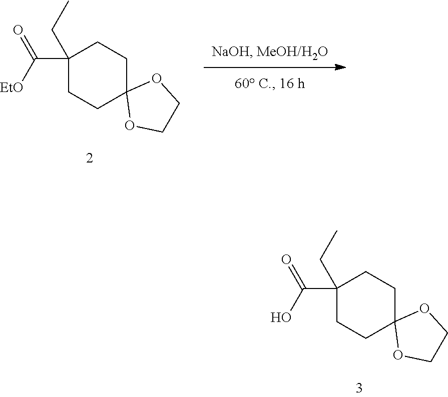 [SMILES: CCOC(=O)C1(CC)CCC2(CC1)OCCO2 ; "NaOH, MeOH/H2O" ; CCC1(C(=O)O)CCC2(CC1)OCCO2]

To a solution of ethyl 8-ethyl-1,4-dioxaspiro[4.5]decane-8-carboxylate (2) (10 g, 41.27 mmol, 1 eq) in MeOH (100 mL) was added a solution of NaOH (6.60 g, 165.08 mmol, 4 eq) in H₂O (40 mL). The mixture was stirred at 60° C. for 16 h, then cooled and concentrated under reduced pressure to give a residue. The residue was diluted with H₂O (50 mL). The pH was adjusted to 5-6 with 3 M HCl (aqueous solution), and then mixture was extracted with EtOAc (100 mL*2). The combined organic layers were washed with brine (50 mL), dried over anhydrous Na₂SO₄, filtered and concentrated under reduced pressure to give the product. Compound 8-ethyl-1,4-dioxaspiro[4.5]decane-8-carboxylic acid (3) (8.4 g, crude) was obtained as a brown solid. ¹H NMR (400 MHz, CHLOROFORM-d) δ=12.06-10.69 (m, 1H), 3.99-3.91 (m, 4H), 2.20-2.10 (m, 2H), 1.74-1.58 (m, 6H), 1.56-1.43 (m, 2H), 0.89 (t, J=7.5 Hz, 3H).

### Procedure for synthesis of tert-butyl N-(8-ethyl-1,4-dioxaspiro[4.5]decan-8-yl)carbamate (4)

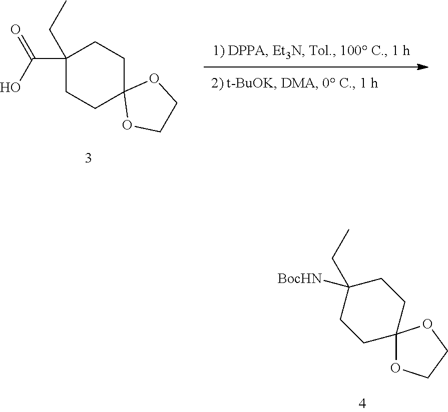

To a solution of 8-ethyl-1,4-dioxaspiro[4.5]decane-8-carboxylic acid (3) (7.4 g, 34.54 mmol, 1 eq) in Tol. (15 mL) was added DPPA (9.50 g, 34.54 mmol, 7.45 mL, 1 eq) and TEA (3.84 g, 37.99 mmol, 5.29 mL, 1.1 eq). The mixture was stirred at 100° C. for 1 hour, then cooled to 20° C., washed with H₂O (20 mL), dried over Na₂SO₄, filtered and concentrated. The residue was dissolved in DMA (15 mL) and cooled to 0° C. tBuOK (4.26 g, 37.99 mmol, 1.1 eq) was then added and the mixture was stirred at 0~15° C. for 1 hour then diluted with aq. sat.NH₄Cl (50 mL) and extracted with EtOAc (30 mL*2). The combined organic layers were washed with sat. aq. NaCl (50 mL), dried over anhydrous Na₂SO₄, filtered and concentrated under reduced pressure to give a residue. The residue was purified by flash silica gel chromatography (ISCO®; 40 g SepaFlash® Silica Flash Column, lenslet 0~13% Ethyl acetate/Petroleum ether gradient @40 mL/min). Compound tert-butyl N-(8-ethyl-1,4-dioxaspiro[4.5]decan-8-yl)carbamate (4) (6.9 g, 24.18 mmol, 70.01% yield) was obtained as a yellow oil. ¹H NMR (400 MHz, CHLOROFORM-d) δ=4.27 (br s, 1H), 3.99-3.90 (m, 4H), 2.05-1.99 (m, 2H), 1.74-1.55 (m, 8H), 1.43 (s, 9H), 0.86-0.81 (m, 3H).

### Procedure for preparation of tert-butyl N-(8-ethyl-1,4-dioxaspiro[4.5]decan-8-yl)-N-methyl-carbamate (5)

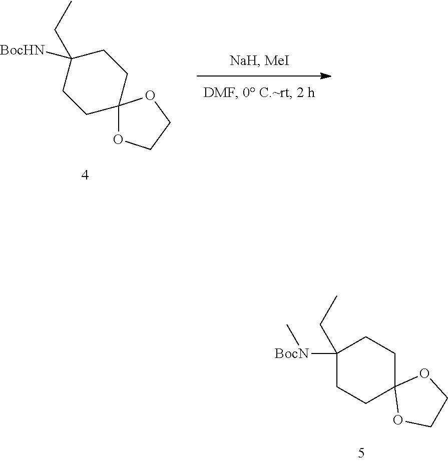

To a solution of tert-butyl N-(8-ethyl-1,4-dioxaspiro[4.5]decan-8-yl)carbamate (4) (6.9 g, 24.18 mmol, 1 eq) in DMF (70 mL) was added in portions NaH (1.26 g, 31.43 mmol, 60% suspension in mineral oil, 1.3 eq) at 0° C. The mixture was stirred at 0° C. for 30 min, and then CH₃I (4.12 g, 29.01 mmol, 1.81 mL, 1.2 eq) was added dropwise at 0° C. The resulting mixture was warmed up slowly to 25° C., stirred for 1.5 h quenched with aq. sat.NH₄Cl (300 mL) and extracted with EtOAc (100 mL*2). The combined organic layers were washed with aq. sat.NaCl (100 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~23% Ethyl acetate/Petroleum ether gradient @40 mL/min). Compound tert-butyl N-(8-ethyl-1,4-dioxaspiro[4.5]decan-8-yl)-N-methyl-carbamate (5) (7 g, 23.38 mmol, 96.70% yield) was obtained as a colorless oil. ¹H NMR (400 MHz, CHLOROFORM-d) δ=3.96-3.93 (m, 4H), 2.85 (s, 3H), 2.43 (br d, J=4.6 Hz, 2H), 1.71-1.60 (m, 8H), 1.47-1.43 (m, 9H), 0.87-0.84 (m, 3H).

### Procedure for preparation of tert-butyl N-(1-ethyl-4-oxo-cyclohexyl)-N-methyl-carbamate (6)

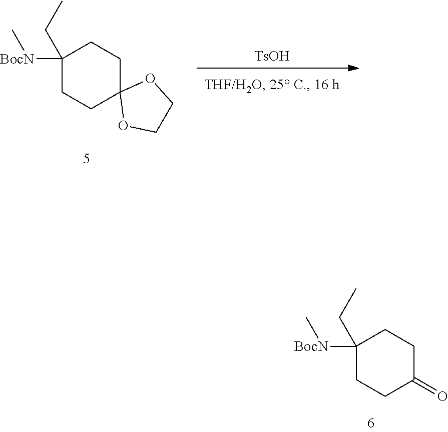 [SMILES: CCC1(N(C)C(=O)OC(C)(C)C)CCC2(CC1)OCCO2 ; "TsOH" ; CCC1(N(C)C(=O)OC(C)(C)C)CCC(=O)CC1]

To a solution of tert-butyl N-(8-ethyl-1,4-dioxaspiro[4.5]decan-8-yl)-N-methyl-carbamate (5) (7 g, 23.38 mmol, 1 eq) in THF (300 mL) and H₂O (200 mL) was added TsOH (8.05 g, 46.76 mmol, 2 eq). The mixture was stirred at 25° C. for 16 hr then diluted with aq. sat.NaHCO₃ (100 mL) and extracted with EtOAc (100 mL*2). The combined organic layers were washed with aq. sat. NaCl (100 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0-35% Ethyl acetate/Petroleum ether gradient @40 mL/min). Compound tert-butyl N-(1-ethyl-4-oxo-cyclohexyl)-N-methyl-carbamate (6) (5.8 g, 22.71 mmol, 97.15% yield) was obtained as a colorless oil.

### Procedure for preparation of tert-butyl N-(4-chloro-1-ethyl-3-formyl-cyclohex-3-en-1-yl)-N-methyl-carbamate (7)

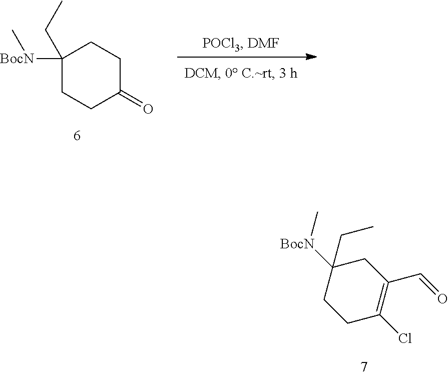

To a solution of DMF (7.47 g, 102.21 mmol, 7.86 mL, 4.5 eq) in DCM (60 mL) was added POCl₃ (13.93 g, 90.85 mmol, 8.47 mL, 4 eq) slowly at 0° C. The mixture was stirred at 0° C. for 1 hour, then tert-butyl N-(1-ethyl-4-oxo-cyclohexyl)-N-methyl-carbamate (6) (5.8 g, 22.71 mmol, 1 eq) in DCM (60 mL) was added, and then the mixture was warmed up slowly to room temperature and stirred for 2 hours. Followed addition of aq. sat.NaHCO₃ (100 mL). The resulting mixture extracted with DCM (100 mL*2) and the combined organic layers were washed with aq. sat.NaCl (50 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~22% Ethyl acetate/Petroleum ether gradient @40 mL/min). Compound tert-butyl N-(4-chloro-1-ethyl-3-formyl-cyclohex-3-en-1-yl)-N-methyl-carbamate (7) (2.9 g, 9.61 mmol, 42.30% yield) was obtained as a yellow oil. ¹H NMR (400 MHz, CHLOROFORM-d) 6=10.19 (s, 1H), 3.06 (br d, J=17.9 Hz, 1H), 2.93-2.82 (m, 1H), 2.72 (s, 3H), 2.68-2.49 (m, 2H), 2.22-2.06 (m, 2H), 1.71 (ddd, J=5.9, 10.8, 13.4 Hz, 1H), 1.55-1.50 (m, 1H), 1.47 (s, 9H), 0.91 (t, J=7.6 Hz, 3H).

### Procedure for preparation of ethyl 5-[tert-butoxycarbonyl(methyl)amino]-5-ethyl-6,7-dihydro-4H-benzothiophene-2-carboxylate (8)

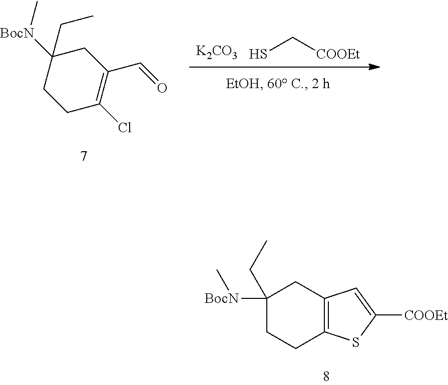

To a solution of tert-butyl N-(4-chloro-1-ethyl-3-formyl-cyclohex-3-en-1-yl)-N-methyl-carbamate (7) (2.9 g, 9.61 mmol, 1 eq) in EtOH (30 mL) was added K₂CO₃ (1.99 g, 14.41 mmol, 1.5 eq) and ethyl 2-sulfanylacetate (1.62 g, 13.45 mmol, 1.47 mL, 1.4 eq). The mixture was stirred at 60° C. for 2 hr, cooled and concentrated. The residue was purified by flash silica gel chromatography (ISCO®; 40 g SepaFlash® Silica Flash Column, eluent 0-15% Ethyl acetate/Petroleum ether gradient @40 mL/min). Compound ethyl 5-[tert-butoxycarbonyl(methyl)amino]-5-ethyl-6,7-dihydro-4H-benzothiophene-2-carboxylate (8) (2 g, 5.44 mmol, 56.64% yield) was obtained as a colorless oil. LCMS: m/z [M+H-Bu]⁺=312.0.

### Procedure for preparation of 5-[tert-butoxycarbonyl(methyl)amino]-5-ethyl-6,7-dihydro-4H-benzothiophene-2-carboxylic acid (9)

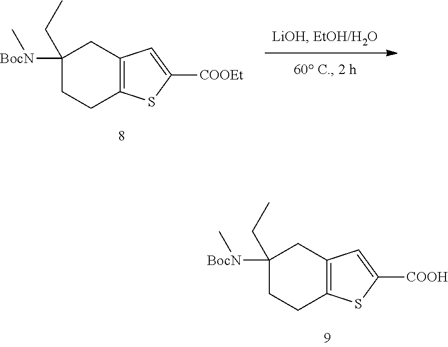 [SMILES: CCOC(=O)c1cc2c(s1)CCC(CC)(N(C)C(=O)OC(C)(C)C)C2 ; "LiOH, EtOH/H2O" ; CCC1(N(C)C(=O)OC(C)(C)C)CCc2sc(C(=O)O)cc2C1]

To a solution of ethyl 5-[tert-butoxycarbonyl(methyl)amino]-5-ethyl-6,7-dihydro-4H-benzothiophene-2-carboxylate (8) (2 g, 5.44 mmol, 1 eq) in MeOH (10 mL) and H₂O (10 mL) was added LiOH·H₂O (913.49 mg, 21.77 mmol, 4 eq). The mixture was stirred at 60° C. for 2 hr. The reaction mixture was then concentrated under vacuum to remove MeOH, diluted with water (20 mL) and extracted with EtOAc (50 mL). The pH of the aqueous phase was adjusted to 3-4 with sat. citric acid aqueous solution. The mixture was extracted with EtOAc (50 mL*3). The combined organic phase was washed with brine (30 mL), dried over anhydrous sodium sulfate, filtered and concentrated under vacuum to afford 5-[tert-butoxycarbonyl(methyl)amino]-5-ethyl-6,7-dihydro-4H-benzothiophene-2-carboxylic acid (9) (1.75 g, 5.16 mmol, 94.73% yield) as a white solid. LCMS: m/z [M+H-Bu]⁺=284.0.

### Procedure for preparation of tert-butyl N-(5-ethyl-6,7-dihydro-4H-benzothiophen-5-yl)-N-methyl-carbamate (10)

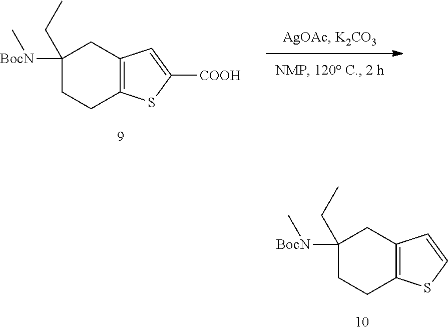

To a solution of 5-[tert-butoxycarbonyl(methyl)amino]-5-ethyl-6,7-dihydro-4H-benzothiophene-2-carboxylic acid (9) (1.75 g, 5.16 mmol, 1 eq) in NMP (30 mL) was added AgOAc (430.25 mg, 2.58 mmol, 0.5 eq) and K₂CO₃ (2.14 g, 15.47 mmol, 3 eq). The mixture was stirred at 120° C. for 2 hr. The reaction mixture was then cooled to rt and diluted with H₂O (200 mL) and extracted with EtOAc (50 mL*2). The combined organic layers were washed with aq. sat.NaCl (50 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~13% Ethyl acetate/Petroleum ether gradient @40 mL/min) to afford tert-butyl N-(5-ethyl-6,7-dihydro-4H-benzothiophen-5-yl)-N-methyl-carbamate (10) (1.3 g, 4.40 mmol, 85.35% yield) as a colorless oil. ¹H NMR (400 MHz, CHLOROFORM-d) δ=7.08 (d, J=5.0 Hz, 1H), 6.73 (d, J=5.0 Hz, 1H), 3.17 (br d, J=17.0 Hz, 1H), 2.89-2.63 (m, 7H), 2.34-2.22 (m, 1H), 1.82 (ddd, J=6.8, 9.0, 13.0 Hz, 1H), 1.61 (dd, J=7.4, 14.1 Hz, 1H), 1.47 (s, 9H), 0.93 (t, J=7.5 Hz, 3H). LCMS: m/z [M-Bu+H]⁺=196.1.

### Procedure for preparation of tert-butyl N-(5-ethyl-6,7-dihydro-4H-benzothiophen-5-yl)-N-methyl-carbamate enantiomer 1 (10a) enantiomer 2 (10b)

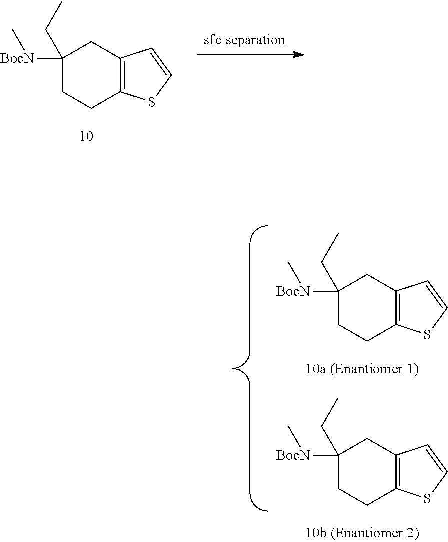 [SMILES: CCC1(N(C)C(=O)OC(C)(C)C)CCc2sccc2C1 ; "sfc separation" ; CCC1(N(C)C(=O)OC(C)(C)C)CCc2sccc2C1 ; CCC1(N(C)C(=O)OC(C)(C)C)CCc2sccc2C1]

Racemic tert-butyl N-(5-ethyl-6,7-dihydro-4H-benzothiophen-5-yl)-N-methyl-carbamate (10) was separated to its enantiomers by SFC (column: DAICEL CHIRALCEL OD (250 mm*30 mm, 10 μm); mobile phase: [Heptane-EtOH]; B %:8%, isocratic elution mode). tert-Butyl N-(5-ethyl-6,7-dihydro-4H-benzothiophen-5-yl)-N-methyl-carbamate enantiomer 1 (10a) (RT=0.657 min, 120 mg, 406.17 mol) and enantiomer 2 (10b) (RT=0.711 min, 140 mg, 473.87 mol) were both obtained as colorless oils.

### Procedure for preparation of 5-ethyl-N-methyl-6,7-dihydro-4H-benzothiophen-5-amine hydrochloride enantiomer 1 (291)

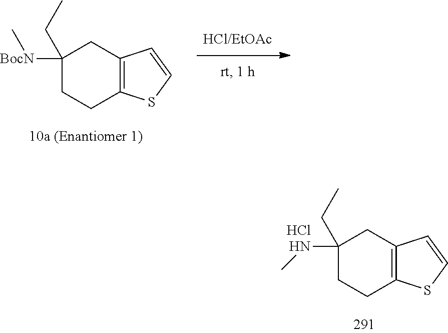 [SMILES: CCC1(N(C)C(=O)OC(C)(C)C)CCc2sccc2C1 ; CCC1(NC)CCc2sccc2C1 ; Cl]

To a solution of tert-butyl N-(5-ethyl-6,7-dihydro-4H-benzothiophen-5-yl)-N-methyl-carbamate enantiomer 1 (10a) (120 mg, 406.17 μmol, 1 eq) in EtOAc (2 mL) was added HCl/EtOAc (2 M, 2 mL, 9.85 eq). The mixture was stirred at 25° C. for 1 hr. The reaction mixture was then filtered and the cake was washed with EtOAc (2 mL) dried and collected. Compound 5-ethyl-N-methyl-6,7-dihydro-4H-benzothiophen-5-amine hydrochloride enantiomer 1 (291) (49.54 mg, 213.73 μmol, 52.62% yield) was obtained as a white solid. ¹H NMR (400 MHz, METHANOL-d4) δ=7.26 (d, J=5.3 Hz, 1H), 6.81 (d, J=5.1 Hz, 1H), 3.00-2.82 (m, 4H), 2.68 (s, 3H), 2.23-2.05 (m, 2H), 1.90-1.73 (m, 2H), 1.03 (t, J=7.6 Hz, 3H). LCMS: m/z [M+H]⁺=196.1.

### Procedure for preparation of 5-ethyl-N-methyl-6,7-dihydro-4H-benzothiophen-5-amine hydrochloride enantiomer 2 (292)

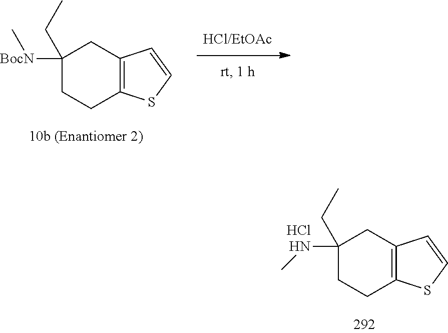 [SMILES: CCC1(N(C)C(=O)OC(C)(C)C)CCc2sccc2C1 ; CCC1(NC)CCc2sccc2C1 ; Cl]

To a solution of tert-butyl N-(5-ethyl-6,7-dihydro-4H-benzothiophen-5-yl)-N-methyl-carbamate enantiomer 2 (10b) (140 mg, 473.87 μmol, 1 eq) in EtOAc (2 mL) was added HCl/EtOAc (2 M, 2 mL, 8.44 eq). The mixture was stirred at 25° C. for 1 hr. The reaction mixture was then filtered and the cake was washed with EtOAc (2 mL) dried and collected. Compound 5-ethyl-N-methyl-6,7-dihydro-4H-benzothiophen-5-amine hydrochloride enantiomer 2 (292) (65.19 mg, 333.75 μmol, 70.43% yield) was obtained as a white solid. ¹H NMR (400 MHz, METHANOL-d4) δ=7.26 (d, J=5.1 Hz, 1H), 6.81 (d, J=5.3 Hz, 1H), 3.01-2.81 (m, 4H), 2.68 (s, 3H), 2.22-2.04 (m, 2H), 1.88-1.73 (m, 2H), 1.03 (t, J=7.6 Hz, 3H). LCMS: m/z [M+H]⁺=196.1.

### Synthetic scheme for preparation of N-methyl-3,4-dihydro-2H-thieno[2,3-b]pyran-3-amine formate salt enantiomer 1 (293) and enantiomer 2 (294)

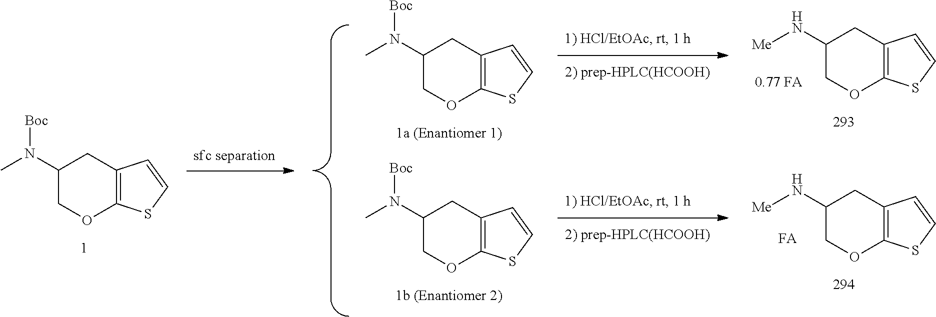

### Procedure for preparation of tert-butyl N-(3,4-dihydro-2H-thieno[2,3-b]pyran-3-yl)-N-methyl-carbamate enantiomer 1 (1a) and enantiomer 2 (1b)

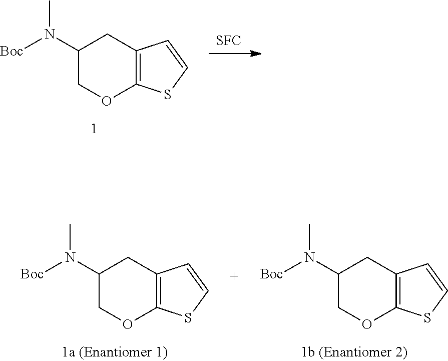

Racemic tert-butyl N-(3,4-dihydro-2H-thieno[2,3-b]pyran-3-yl)-N-methyl-carbamate (1) (600 mg) was separated to its enantiomers by SFC (column: DAICEL CHIRALPAK IG (250 mm*50 mm, 10 μm); mobile phase: [CO₂-EtOH (0.1% NH₃H₂O)]; B %:20%, isocratic elution mode) to afford tert-butyl N-(3,4-dihydro-2H-thieno[2,3-b]pyran-3-yl)-N-methyl-carbamate enantiomer 1 (1a) (RT=1.029 min, 190 mg) and enantiomer 2 (1b) (RT=1.274 min, 200 mg), both as colorless oils.

### Procedure for preparation of N-methyl-3,4-dihydro-2H-thieno[2,3-b]pyran-3-amine formate salt enantiomer 1 (293)

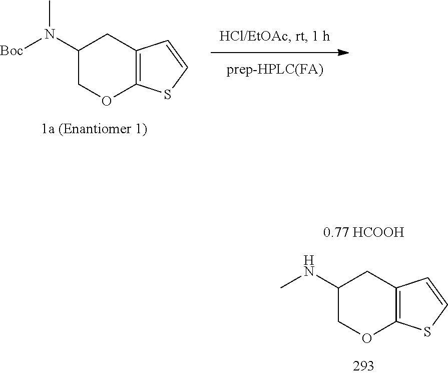 [SMILES: CN(C(=O)OC(C)(C)C)C1COc2sccc2C1 ; CNC1COc2sccc2C1]

To a solution of tert-butyl N-(3,4-dihydro-2H-thieno[2,3-b]pyran-3-yl)-N-methyl-carbamate enantiomer 1 (1a) (100 mg, 371.25 μmol, 1 eq) in EtOAc (2 mL) was added HCl/EtOAc (2 M, 2 mL, 10.77 eq). The mixture was stirred at 25° C. for 1 hr. Then the solvent was removed. The residue was purified by prep-HPLC (column: C18 150×mm; mobile phase: [water(FA)-ACN]; gradient:0%-20% B over 7 min). Compound N-methyl-3,4-dihydro-2H-thieno[2,3-b]pyran-3-amine enantiomer 1 formate salt (293) (20.48 mg, 93.00 μmol, 25.05% yield) was obtained as a gray solid. ¹H NMR (400 MHz, METHANOL-d4) δ=8.61-8.40 (m, 1H), 6.76-6.58 (m, 2H), 4.51 (br d, J=10.0 Hz, 1H), 4.27 (br d, J=12.1 Hz, 1H), 3.80-3.61 (m, 1H), 3.21-3.06 (m, 1H), 2.94-2.71 (m, 4H). LC-MS: m/z [M+H]⁺=170.2

### Procedure for preparation of N-methyl-3,4-dihydro-2H-thieno[2,3-b]pyran-3-amine formate salt enantiomer 2 (294)

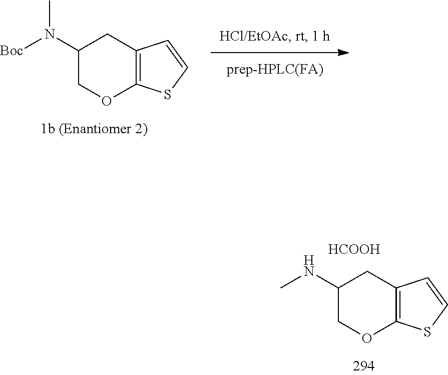 [SMILES: CN(C(=O)OC(C)(C)C)C1COc2sccc2C1 ; "HCOOH" ; CNC1COc2sccc2C1]

To a solution of tert-butyl N-(3,4-dihydro-2H-thieno[2,3-b]pyran-3-yl)-N-methyl-carbamate enantiomer 2 (1b) (100 mg, 371.25 μmol, 1 eq) in EtOAc (2 mL) was added HCl/EtOAc (2 M, 2 mL, 10.77 eq). The mixture was stirred at 25° C. for 1 hr then concentrated. The residue was purified by prep-HPLC (column: C18 150×30 mm; mobile phase: [water-ACN (50% THF)]; gradient:0%-20% B over 7 min). Compound N-methyl-3,4-dihydro-2H-thieno[2,3-b]pyran-3-amine formate salt enantiomer 2 (294) (10.49 mg, 47.55 μmol, 12.81% yield) was obtained as a yellow solid. ¹H NMR (400 MHz, METHANOL-d4) δ=8.48 (s, 1H), 6.64 (s, 2H), 4.48 (br dd, J=2.3, 12.1 Hz, 1H), 4.27 (d, J=11.8 Hz, 1H), 3.66 (br d, J=3.4 Hz, 1H), 3.12 (dd, J=5.7, 17.1 Hz, 1H), 2.87-2.72 (m, 4H). LC-MS: m/z [M+H]⁺=170.2

### Synthetic scheme for preparation of 3-methyl-6,7-dihydro-5H-thieno[3,2-b]pyran-6-amine hydrochloride enantiomer 1 (295) and enantiomer 2 (296)

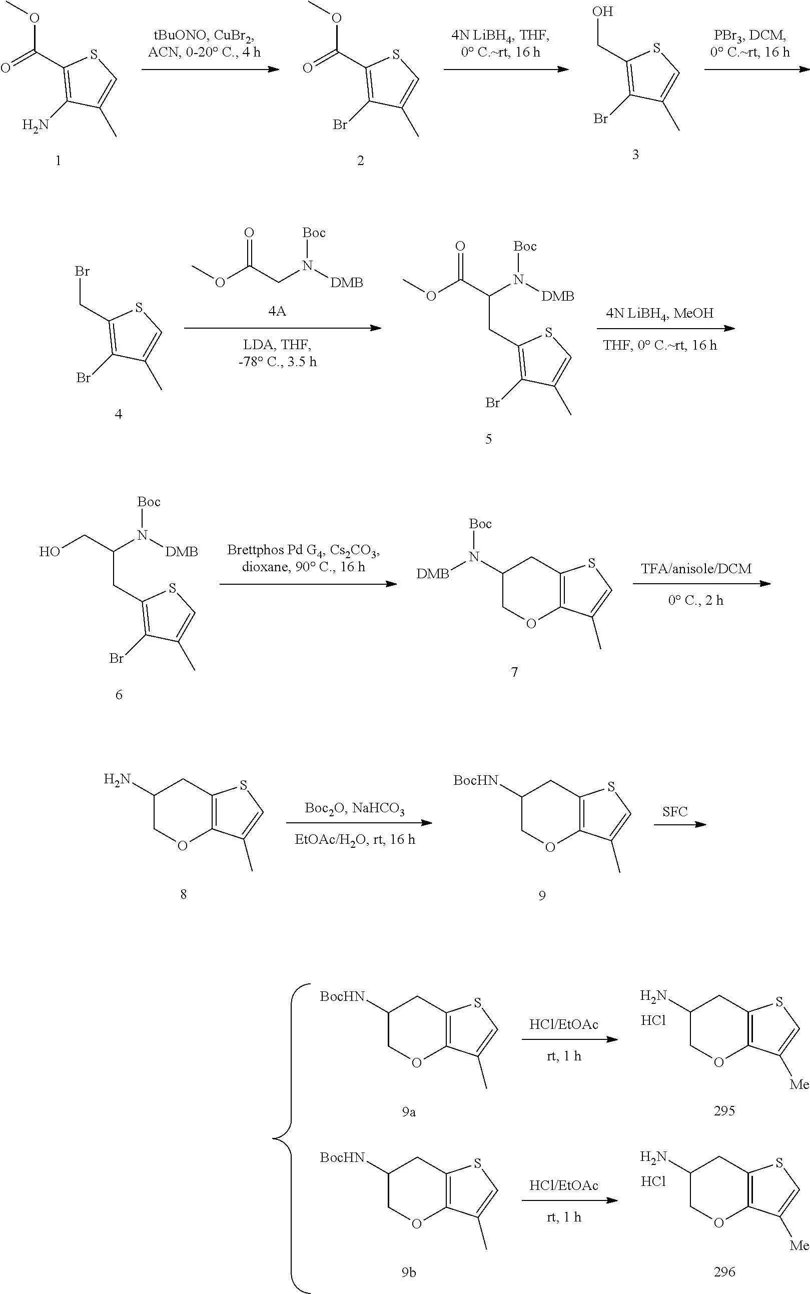

### Procedure for preparation of methyl 3-bromo-4-methyl-thiophene-2-carboxylate (2)

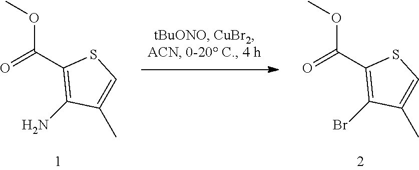

To a solution of CuBr₂ (39.14 g, 175.22 mmol, 8.20 mL, 1.2 eq) in CH₃CN (200 mL) was added t-BuONO (19.57 g, 189.82 mmol, 22.58 mL, 1.3 eq) at 0° C. Then it was added a solution of methyl 3-amino-4-methyl-thiophene-2-carboxylate (1) (25 g, 146.01 mmol, 1 eq) in CH₃CN (100 mL) dropwise over 0.5 h at 0° C. The mixture was then warmed up and stirred at 25° C. for 3.5 h. The reaction mixture was then filtered and the filtrate was concentrated under reduced pressure to give a residue. The residue was purified by flash silica gel chromatography (ISCO®; 120 g SepaFlash® Silica Flash Column, eluent 0~13% Ethyl acetate/Petroleum ether gradient @60 mL/min). Compound methyl 3-bromo-4-methyl-thiophene-2-carboxylate (2) (26 g, 110.59 mmol, 75.74% yield) was obtained as a white solid. ¹H NMR (400 MHz, CHLOROFORM-d) δ=7.23 (s, 1H), 3.90 (s, 3H), 2.28 (s, 3H)

### Procedure for preparation of (3-bromo-4-methyl-2-thienyl)methanol (3)

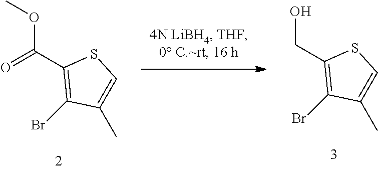

To a solution of methyl 3-bromo-4-methyl-thiophene-2-carboxylate (2) (26 g, 110.59 mmol, 1 eq) in THF (300 mL) was added LiBH₄ (4 M in THF, 55.30 mL, 2 eq) first and then MeOH (5 mL) dropwise at 0° C. The mixture was then stirred at 25° C. for 16 hr and quenched by addition of aq. sat. NH₄Cl (300 mL) at 0° C. under N₂, and extracted with EtOAc (300 mL*2). The combined organic layers were washed with aq. sat.NaCl (300 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~10% Ethyl acetate/Petroleum ether gradient @50 mL/min). Compound (3-bromo-4-methyl-2-thienyl)methanol (3) (21.3 g, 102.85 mmol, 93.00% yield) was obtained as a yellowish oil.

### Procedure for preparation of 3-bromo-2-(bromomethyl)-4-methyl-thiophene (4)

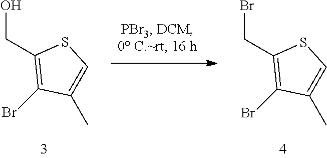

To a solution of (3-bromo-4-methyl-2-thienyl)methanol (3) (11 g, 53.12 mmol, 1 eq) in DCM (150 mL) was added PBr₃ (28.76 g, 106.24 mmol, 2 eq) dropwise at 0° C. The mixture was then stirred at 25° C. for 16 hr and then quenched by addition of aq. sat.NaHCO₃ (400 mL) at 0° C. The mixture was then extracted with EtOAc (200 mL*2). The combined organic layers were washed with brine (200 mL), dried over anhydrous Na₂SO₄, filtered and concentrated. Compound 3-bromo-2-(bromomethyl)-4-methyl-thiophene (4) (12.3 g, crude) was obtained as a yellow oil. ¹H NMR (400 MHz, CHLOROFORM-d) δ=7.05 (s, 1H), 4.69 (s, 2H), 2.23 (s, 3H)

### Procedure for preparation of methyl 3-(3-bromo-4-methyl-2-thienyl)-2-[tert-butoxycarbonyl-[(2,4-dimethoxyphenyl)methyl]amino]propanoate (5)

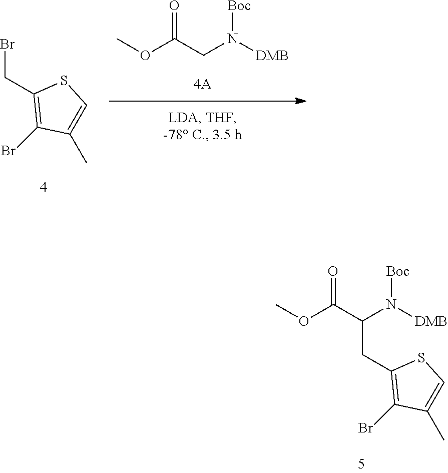

To a solution of LDA (2 M in THF, 19.45 mL, 1.2 eq) in THF (100 mL) at −78° C. was added a solution of methyl 2-[tert-butoxycarbonyl-[(2,4-dimethoxyphenyl)methyl]amino]acetate (11 g, 32.41 mmol, 1 eq) in THF (30 mL) slowly under N₂. After 1 h, a solution of 3-bromo-2-(bromomethyl)-4-methyl-thiophene (4) (10.50 g, 38.89 mmol, 1.2 eq) in THF (20 mL) was added. The mixture was stirred at −78° C. for 2.5 hr, warmed up to 0° C., quenched with aq. sat.NH₄Cl (100 mL), and extracted with EtOAc (100 mL*2). The combined organic layers were washed with aq. sat.NaCl (100 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~12% Ethyl acetate/Petroleum ether gradient @60 mL/min). Compound methyl 3-(3-bromo-4-methyl-2-thienyl)-2-[tert-butoxycarbonyl-[(2,4-dimethoxyphenyl)methyl]amino]propanoate (5) (13.8 g, 26.11 mmol, 80.57% yield) was obtained as a pink oil.

### Procedure for preparation of tert-butyl N-[1-[(3-bromo-4-methyl-2-thienyl)methyl]-2-hydroxy-ethyl]-N-[(2,4-dimethoxyphenyl)methyl]carbamate (6)

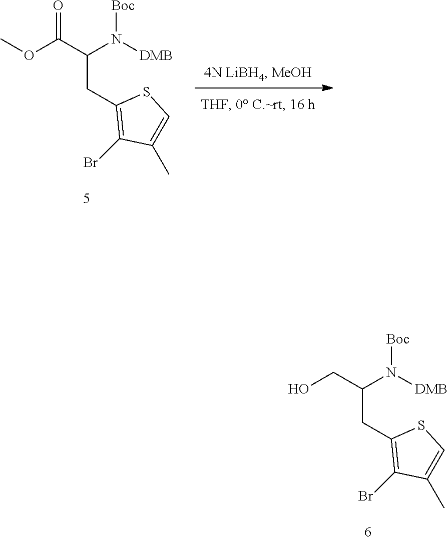

To a solution of methyl 3-(3-bromo-4-methyl-2-thienyl)-2-[tert-butoxycarbonyl-[(2,4-dimethoxyphenyl)methyl]amino]propanoate (5) (13.8 g, 26.11 mmol, 1 eq) in THF (150 mL) was added LiBH₄ (4 M, 26.11 mL, 4 eq) and then MeOH (5 mL) at 0° C. under nitrogen. Then the mixture was allowed to naturally warm up to rt and then stirred for 16 h under N₂. The reaction mixture was then quenched with aq. sat. NH₄Cl 500 mL and extracted with EtOAc (200 mL*2). The combined organic layers were washed with aq. sat.NaCl (100 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0~30% Ethyl acetate/Petroleum ether gradient @40 mL/min). Compound tert-butyl N-[1-[(3-bromo-4-methyl-2-thienyl)methyl]-2-hydroxy-ethyl]-N-[(2,4-dimethoxyphenyl)methyl]carbamate (6) (8 g, 15.99 mmol, 61.22% yield) was obtained as a colorless oil. LC-MS: m/z [M+H-tBu]⁺=446.0

### Procedure for preparation of tert-butyl N-[(2,4-dimethoxyphenyl)methyl]-N-(3-methyl-6,7-dihydro-5H-thieno[3,2-b]pyran-6-yl)carbamate (7)

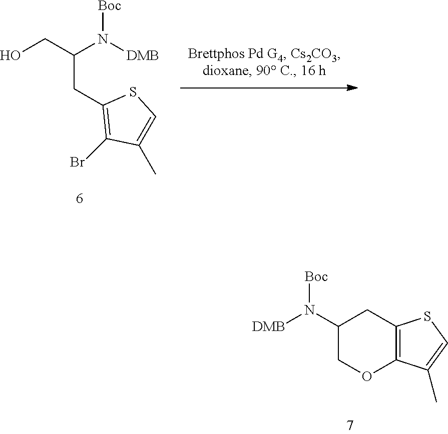

A mixture of tert-butyl N-[1-[(3-bromo-4-methyl-2-thienyl)methyl]-2-hydroxy-ethyl]-N-[(2,4-dimethoxyphenyl)methyl]carbamate (6) (8 g, 15.99 mmol, 1 eq), Brettphos Pd G4 (1.18 g, 1.28 mmol, 0.08 eq) and Cs₂CO₃ (15.63 g, 47.96 mmol, 3 eq) in dioxane (800 mL) was degassed and purged with N₂ and then stirred at 90° C. for 16 hr. The mixture was then filtered and the filtrate was concentrated under reduced pressure. The residue was purified by flash silica gel chromatography (ISCO®; 80 g SepaFlash® Silica Flash Column, eluent 0~12% Ethyl acetate/Petroleum ether gradient @40 mL/min). Compound tert-butyl N-[(2,4-dimethoxyphenyl)methyl]-N-(3-methyl-6,7-dihydro-5H-thieno[3,2-b]pyran-6-yl)carbamate (7) (5.4 g, 12.87 mmol, 80.52% yield) was obtained as a yellow oil. ¹H NMR (400 MHz, CHLOROFORM-d) δ=7.18-7.08 (m, 1H), 6.59 (s, 1H), 6.47 (dd, J=2.3, 8.3 Hz, 1H), 6.43 (d, J=2.4 Hz, 1H), 4.53-4.17 (m, 3H), 4.13-4.06 (m, 2H), 3.80 (d, J=13.0 Hz, 6H), 3.05 (dd, J=10.4, 15.3 Hz, 1H), 2.83-2.62 (m, 1H), 2.01 (s, 3H), 1.54-1.41 (m, 9H). LC-MS: m/z [M+Na]⁺=442.0

### Procedure for preparation of 3-methyl-6,7-dihydro-5H-thieno[3,2-b]pyran-6-amine trifluoracetic acid salt (8)

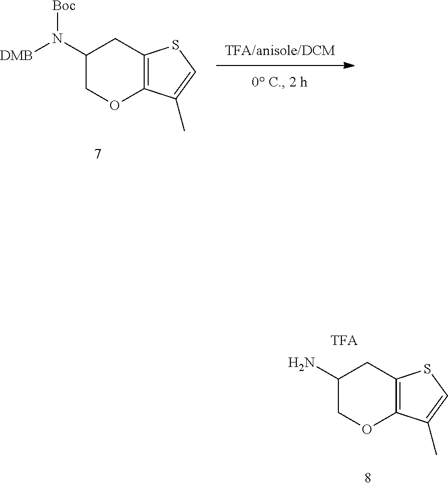

To a solution of tert-butyl N-[(2,4-dimethoxyphenyl)methyl]-N-(3-methyl-6,7-dihydro-5H-thieno[3,2-b]pyran-6-yl)carbamate (7) (5.4 g, 12.87 mmol, 1 eq) in anisole (5.97 g, 55.21 mmol, 6 mL, 4.29 eq) and DCM (10 mL) was added TFA (6 mL) at 0° C. The mixture was stirred at 0° C. for 2 h and then poured into 100 mL of ice-water carefully. The aqueous phase was washed with PE (30 ml). The product 3-methyl-6,7-dihydro-5H-thieno[3,2-b]pyran-6-amine trifluoroacetic acid salt (2.1 g, crude) (8) was obtained as a solution in H₂O (100 ml) LC-MS: m/z [M+H]⁺=170.1.

### Procedure for preparation of tert-butyl N-(3-methyl-6,7-dihydro-5H-thieno[3,2-b]pyran-6-yl)carbamate (9)

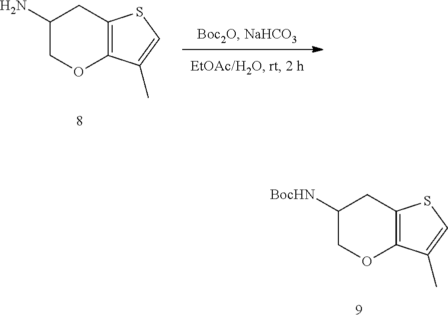 [SMILES: Cc1csc2c1OCC(N)C2 ; Cc1csc2c1OCC(NC(=O)OC(C)(C)C)C2]

To a mixture of 3-methyl-6,7-dihydro-5H-thieno[3,2-b]pyran-6-amine (8) (2.1 g, 12.41 mmol, 1 eq) in EtOAc (50 mL) and H₂O was added NaHCO₃ (1.04 g, 12.41 mmol, 1 eq) and (Boc)₂O (5.42 g, 24.82 mmol, 5.70 mL, 2 eq). The mixture was stirred at rt for 2 hr and then extracted with EtOAc (30 mL*2). The combined organic layers were washed with aq. sat.NaCl (50 mL), dried over anhydrous Na₂SO₄, filtered and concentrated under reduced pressure. The residue was purified by flash silica gel chromatography (ISCO®; 40 g SepaFlash® Silica Flash Column, eluent 0~12% Ethyl acetate/Petroleum ether gradient @40 mL/min). Compound tert-butyl N-(3-methyl-6,7-dihydro-5H-thieno[3,2-b]pyran-6-yl)carbamate (9) (1.3 g, 4.83 mmol, 38.90% yield) was obtained as a colorless oil. ¹H NMR (400 MHz, CHLOROFORM-d) δ=6.67 (s, 1H), 4.99 (br d, J=6.8 Hz, 1H), 4.21 (br d, J=18.0 Hz, 2H), 4.07-4.00 (m, 1H), 3.07 (br dd, J=5.2, 16.3 Hz, 1H), 2.70 (br d, J=16.3 Hz, 1H), 2.08 (s, 3H), 1.45 (s, 9H). LC-MS: m/z [M-tBu+H]⁺=214.0

### Procedure for preparation of tert-butyl N-(3-methyl-6,7-dihydro-5H-thieno[3,2-b]pyran-6-yl)carbamate enantiomer 1 (9a) and enantiomer 2 (9b)

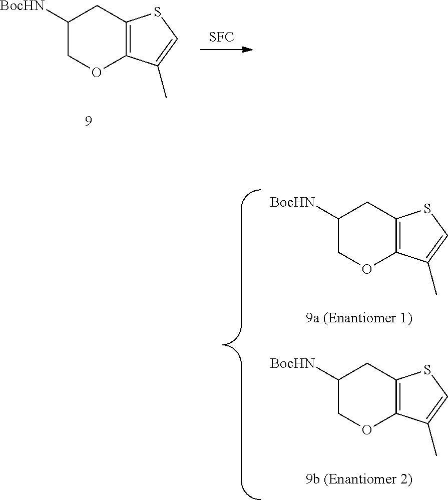

Racemic tert-butyl N-(3-methyl-6,7-dihydro-5H-thieno[3,2-b]pyran-6-yl)carbamate (9) was separated to its enantiomers by SFC (column: DAICEL CHIRALPAK IG (250 mm*30 mm, 10 um); mobile phase: [CO₂-MeOH (0.1% NH₃H₂O)]; B %:12%, isocratic elution mode) to afford the 1^{st} enantiomer (9a) (RT=0.978 min, 130 mg) and the 2nd enantiomer (9b) (RT=1.214 min, 140 mg) both as colorless oils.

### Procedure for preparation of 3-methyl-6,7-dihydro-5H-thieno[3,2-b]pyran-6-amine hydrochloride enantiomer 1 (295)

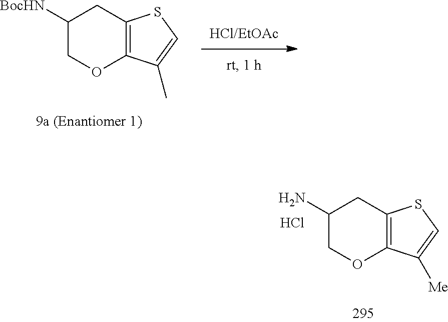 [SMILES: Cc1csc2c1OCC(NC(=O)OC(C)(C)C)C2 ; Cl ; Cc1csc2c1OCC(N)C2]

To a solution of tert-butyl N-(3-methyl-6,7-dihydro-5H-thieno[3,2-b]pyran-6-yl)carbamate enantiomer 1 (9a) (40 mg, 148.50 μmol, 1 eq) in EtOAc (1 mL) was added HCl/EtOAc (2 M, 2 mL, 26.94 eq). The mixture was stirred at rt for 1 hr and then concentrated. Compound 3-methyl-6,7-dihydro-5H-thieno[3,2-b]pyran-6-amine hydrochloride enantiomer 1 (295) (26.17 mg, 124.99 μmol, 84.17% yield) was obtained as a white solid. ¹H NMR (400 MHz, METHANOL-d4) δ=6.84 (s, 1H), 4.38-4.28 (m, 1H), 4.18 (d, J=12.0 Hz, 1H), 3.90 (br s, 1H), 3.26 (d, J=5.5 Hz, 1H), 2.85 (br d, J=17.4 Hz, 1H), 2.07 (d, J=1.0 Hz, 3H). LC-MS: m/z [M+H]⁺=170.1

### Procedure for preparation of 3-methyl-6,7-dihydro-5H-thieno[3,2-b]pyran-6-amine hydrochloride enantiomer 2 (296)

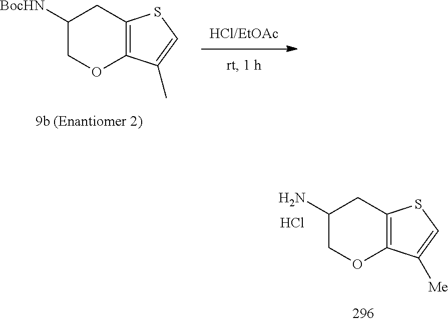 [SMILES: Cc1csc2c1OCC(NC(=O)OC(C)(C)C)C2 ; Cl ; Cc1csc2c1OCC(N)C2]

To a solution of tert-butyl N-(3-methyl-6,7-dihydro-5H-thieno[3,2-b]pyran-6-yl)carbamate enantiomer 2 (9b) (40 mg, 148.50 μmol, 1 eq) in EtOAc (1 mL) was added HCl/EtOAc (2 M, 2 mL, 26.94 eq). The mixture was stirred at rt for 1 hr and then concentrated. Compound 3-methyl-6,7-dihydro-5H-thieno[3,2-b]pyran-6-amine hydrochloride enantiomer 2 (296) (27.56 mg, 131.96 μmol, 88.86% yield) was obtained as a white solid. ¹H NMR (400 MHz, METHANOL-d4) δ=6.86 (s, 1H), 4.42-4.28 (m, 1H), 4.20 (d, J=12.0 Hz, 1H), 3.92 (br d, J=2.6 Hz, 1H), 3.28 (d, J=5.6 Hz, 1H), 2.87 (br d, J=17.0 Hz, 1H), 2.09 (d, J=0.9 Hz, 3H). LC-MS: m/z [M+H]⁺=170.1

### Synthetic scheme for preparation of N, 3-dimethyl-6,7-dihydro-5H-thieno[3,2-b]pyran-6-amine hydrochloride enantiomer 1 (297) and enantiomer 2 (298)

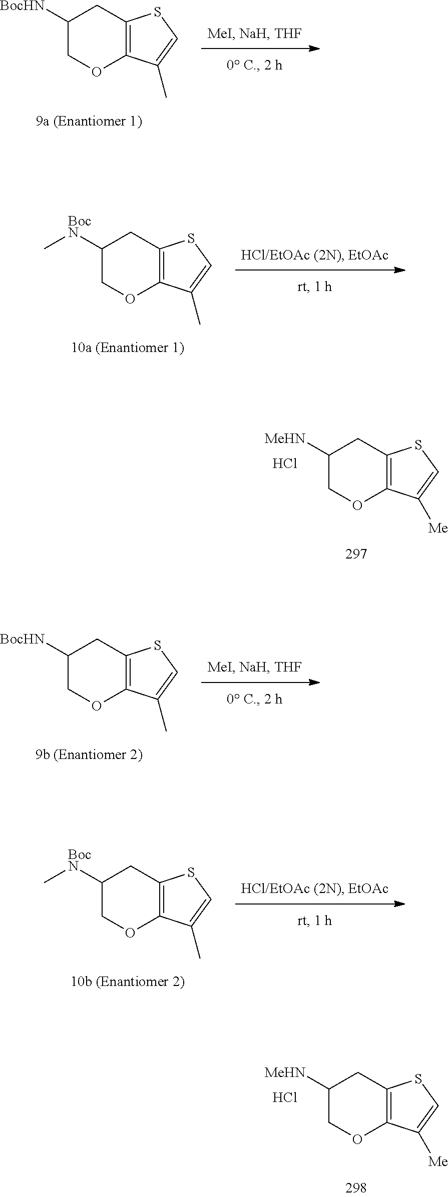

### Procedure for preparation of tert-butyl N-methyl-N-(3-methyl-6,7-dihydro-5H-thieno[3,2-b]pyran-6-yl)carbamate enantiomer 1 (10a)

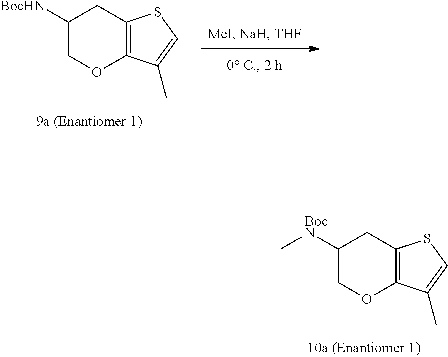 [SMILES: Cc1csc2c1OCC(NC(=O)OC(C)(C)C)C2 ; Cc1csc2c1OCC(N(C)C(=O)OC(C)(C)C)C2]

To a solution of tert-butyl N-(3-methyl-6,7-dihydro-5H-thieno[3,2-b]pyran-6-yl)carbamate enantiomer 1 (9a) (90 mg, 334.13 μmol, 1 eq) in DMF (2 mL) was added in portions NaH (17.37 mg, 434.36 μmol, 60% suspension in mineral oil, 1.3 eq) at 0° C. The mixture was stirred for 0.5 hr, and then CH₃I (56.91 mg, 400.95 μmol, 24.96 μL, 1.2 eq) was added. After stirring at 0° C. for 1.5 hr the mixture was quenched with aq. sat.NH₄Cl (10 mL) and extracted with EtOAc (10 mL*2). The combined organic layers were washed with aq. sat.NaCl (10 mL), dried over anhydrous Na₂SO₄, filtered and concentrated. The residue was purified by prep-TLC (Petroleum ether:Ethyl acetate=3:1). Compound tert-butyl N-methyl-N-(3-methyl-6,7-dihydro-5H-thieno[3,2-b]pyran-6-yl)carbamate enantiomer 1 (10a) (70 mg, 247.01 μmol, 73.93% yield) was obtained as a colorless oil. LC-MS: m/z [M-Bu+1]⁺=228.0

### Procedure for preparation of N, 3-dimethyl-6,7-dihydro-5H-thieno[3,2-b]pyran-6-amine hydrochloride enantiomer 1 (297)

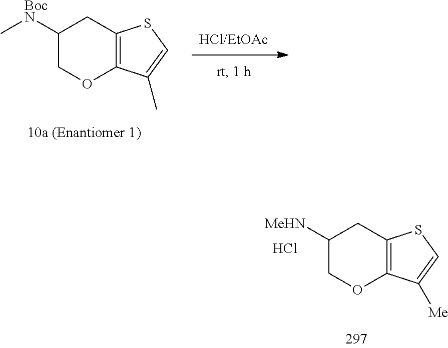 [SMILES: Cc1csc2c1OCC(N(C)C(=O)OC(C)(C)C)C2 ; Cl ; CNC1COc2c(C)csc2C1]

To a solution of tert-butyl N-methyl-N-(3-methyl-6,7-dihydro-5H-thieno[3,2-b]pyran-6-yl)carbamate enantiomer 1 (10a) (70 mg, 247.01 μmol, 1 eq) in EtOAc (1 mL) was added HCl/EtOAc (2 M, 2 mL, 16.19 eq). The mixture was stirred at rt for 1 hr and then concentrated. Compound N, 3-dimethyl-6,7-dihydro-5H-thieno[3,2-b]pyran-6-amine hydrochloride enantiomer 1 (297) (49.47 mg, 223.20 μmol, 90.36% yield) was obtained as a white solid. ¹H NMR (400 MHz, METHANOL-d4) δ=6.87 (s, 1H), 4.61-4.51 (m, 1H), 4.22 (d, J=12.4 Hz, 1H), 3.83 (br s, 1H), 3.09-2.96 (m, 1H), 2.83 (s, 3H), 2.09 (d, J=0.9 Hz, 3H). LC-MS: m/z [M-Bu+H]⁺=184.0

### Procedure for preparation of tert-butyl N-methyl-N-(3-methyl-6,7-dihydro-5H-thieno[3,2-b]pyran-6-yl)carbamate enantiomer 2 (10b)

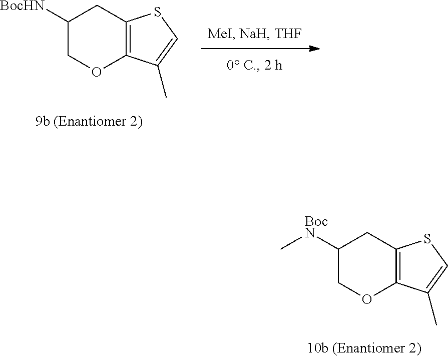 [SMILES: Cc1csc2c1OCC(NC(=O)OC(C)(C)C)C2 ; Cc1csc2c1OCC(N(C)C(=O)OC(C)(C)C)C2]

To a solution of tert-butyl N-(3-methyl-6,7-dihydro-5H-thieno[3,2-b]pyran-6-yl)carbamate enantiomer 2 (9b) (100 mg, 371.25 μmol, 1 eq) in DMF (2 mL) was added in portions NaH (19.30 mg, 482.63 μmol, 60% suspension in mineral oil, 1.3 eq) at 0° C. The mixture was stirred at 0° C. for 0.5 hr, then CH₃I (63.23 mg, 445.50 μmol, 27.73 μL, 1.2 eq) was added. After stirring at 0° C. for 1.5 hr the reaction mixture was quenched with aq. sat.NH₄Cl (10 mL) and extracted with EtOAc (10 mL*2). The combined organic layers were washed with aq. sat.NaCl (10 mL), dried over anhydrous Na₂SO₄, filtered and concentrated under reduced pressure. The residue was purified by prep-TLC (Petroleum ether:Ethyl acetate=3:1). Compound tert-butyl N-methyl-N-(3-methyl-6,7-dihydro-5H-thieno[3,2-b]pyran-6-yl)carbamate enantiomer 2 (10b) (85 mg, 299.94 μmol, 80.79% yield) was obtained as a colorless oil. LC-MS: m/z [M-Bu+1]⁺=228.0

### Procedure for preparation of N, 3-dimethyl-6,7-dihydro-5H-thieno[3,2-b]pyran-6-amine hydrochloride enantiomer 2 (298)

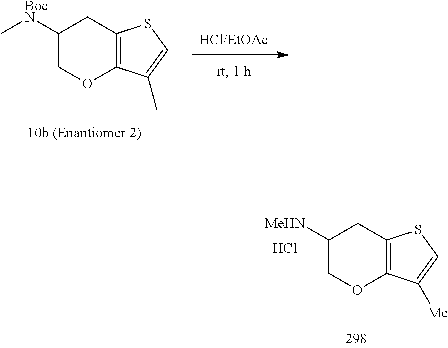 [SMILES: Cc1csc2c1OCC(N(C)C(=O)OC(C)(C)C)C2 ; Cl ; CNC1COc2c(C)csc2C1]

To a solution of tert-butyl N-methyl-N-(3-methyl-6,7-dihydro-5H-thieno[3,2-b]pyran-6-yl)carbamate enantiomer 2 (10b) (85 mg, 299.94 μmol, 1 eq) in EtOAc (1 mL) was added HCl/EtOAc (2 M, 149.97 μL, 1 eq). The mixture was stirred at rt for 1 hr and then evaporated. Compound N, 3-dimethyl-6,7-dihydro-5H-thieno[3,2-b]pyran-6-amine hydrochloride enantiomer 2 (298) (62.13 mg, 280.86 μmol, 93.64% yield) was obtained as a white solid. ¹H NMR (400 MHz, METHANOL-d4) δ=6.87 (s, 1H), 4.62-4.50 (m, 1H), 4.22 (br d, J=12.5 Hz, 1H), 3.89-3.78 (m, 1H), 3.01 (br d, J=18.5 Hz, 1H), 2.92-2.80 (m, 3H), 2.09 (s, 3H). LC-MS: m/z [M-Bu+H]⁺=184.0

### Synthetic scheme for preparation of N, 5-dimethyl-3,4-dihydro-2H-thieno[2,3-b]pyran-3-amine hydrochloride (308)

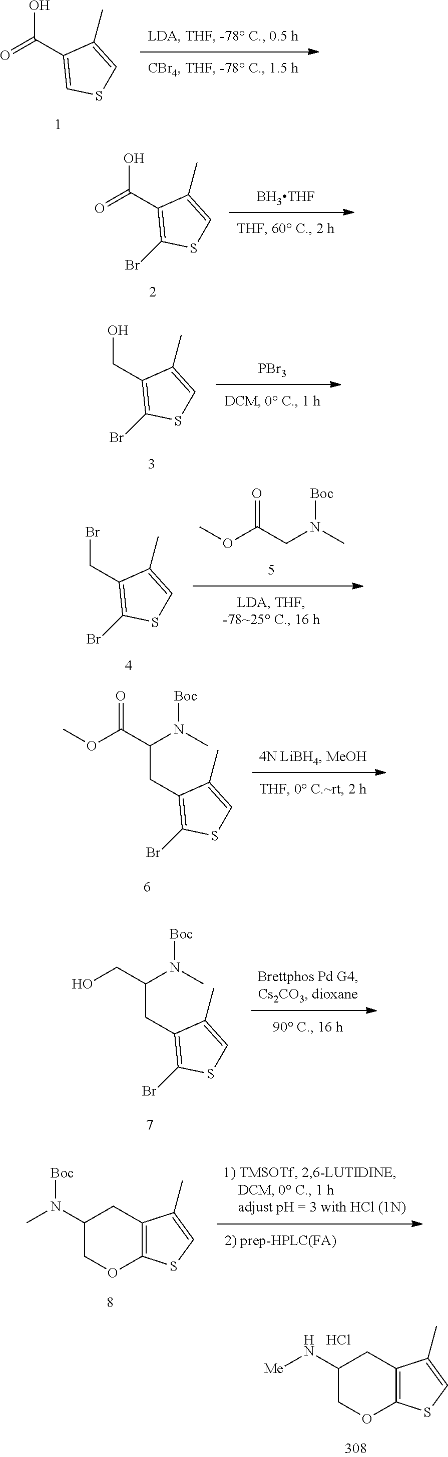

### Procedure for preparation of 2-bromo-4-methyl-thiophene-3-carboxylic acid (2)

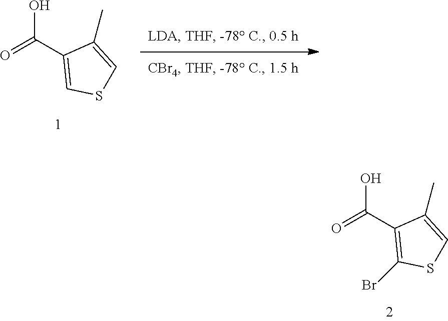

To a solution of LDA (2 M, 63.30 mL, 2 eq) at −78° C. was added a solution of 4-methylthiophene-3-carboxylic acid (1) (9 g, 63.30 mmol, 1 eq) in THF (150 mL), and then the mixture was stirred at −78° C. for 0.5 h. Then added a solution of CBr₄ (23.09 g, 69.63 mmol, 1.1 eq) in THF (150 mL). The resulting mixture was then stirred at −78° C. for 1.5 h and quenched with aq. sat.NH₄Cl (200 mL). The mixture extracted with EtOAc (100 mL*2) and the combined organic layers were washed with aq. sat.NaCl (100 mL), dried over anhydrous Na₂SO₄, filtered and concentrated under reduced pressure to give a residue. The residue was purified by flash silica gel chromatography (ISCO®; 80 g SepaFlash® Silica Flash Column, eluent 0-20% Ethyl acetate/Petroleum ether gradient @40 mL/min). Compound 2-bromo-4-methyl-thiophene-3-carboxylic acid (2) crude (11 g as mixture containing 50% of starting acid 1)) was obtained as a yellow solid. LCMS: m/z [M+H]⁺=222.9.

### Procedure for preparation of (2-bromo-4-methyl-3-thienyl)methanol (3)

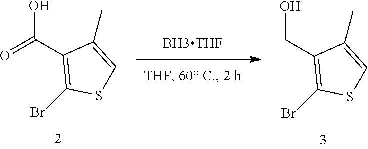

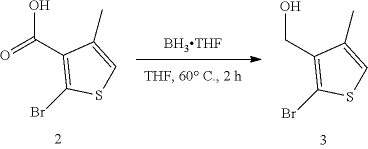

To a solution of crude 2-bromo-4-methyl-thiophene-3-carboxylic acid (2) (11 g) in THF (150 mL) was added BH₃-Me₂S (10 M, 10.86 mL, 2 eq) at 0° C. The mixture was stirred at 60° C. for 2 hr. The reaction was then cooled, 60 ml of methanol added and the resulting mixture was stirred at 70° C. for 1 hr, then cooled and concentrated under vacuum. The residue was purified by flash silica gel chromatography (ISCO®; 40 g SepaFlash® Silica Flash Column, eluent 0~35% Ethyl acetate/Petroleum ether gradient @40 mL/min). Compound (2-bromo-4-methyl-3-thienyl)methanol (3) (9.2 g) was obtained as a colorless oil. ¹H NMR (400 MHz, CHLOROFORM-d) δ=6.91 (s, 1H), 4.61 (s, 2H), 2.32 (d, J=1.0 Hz, 3H), 1.50 (br s, 1H)

### Procedure for preparation of 2-bromo-3-(bromomethyl)-4-methyl-thiophene (4)

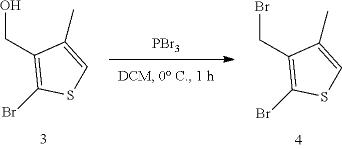

To a solution of (2-bromo-4-methyl-3-thienyl)methanol (3) (9.2 g) in DCM (80 mL) was added PBr₃ (18.04 g, 66.64 mmol, 1.5 eq). The mixture was stirred at 0° C. for 1 hr then diluted with aq. sat.NaHCO₃ (200 mL) and extracted with DCM (100 mL*2). The combined organic layers were washed with aq. sat.NaCl (100 mL), dried over anhydrous Na₂SO₄, filtered and concentrated under reduced pressure. Compound 2-bromo-3-(bromomethyl)-4-methyl-thiophene (4) (12 g, crude) was obtained as a colorless oil. ¹H NMR (400 MHz, CHLOROFORM-d) δ=6.92 (s, 1H), 4.44 (s, 2H), 2.31 (d, J=1.0 Hz, 3H)

### Procedure for preparation of methyl 3-(2-bromo-4-methyl-3-thienyl)-2-[tert-butoxycarbonyl(methyl)amino]propanoate (6)

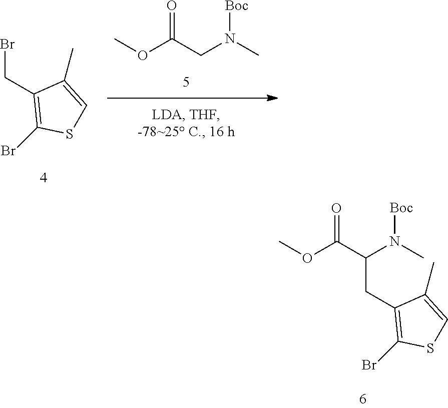

To a solution of LDA (2 M in THF, 33.34 mL, 1.5 eq) in THF (150 mL) under nitrogen was added dropwise methyl 2-[tert-butoxycarbonyl(methyl)amino]acetate (13.55 g, 66.67 mmol, 1.5 eq) as solution in THF (50 mL) at −78° C., The mixture was stirred at −78° C. for 1 hr, and then 2-bromo-3-(bromomethyl)-4-methyl-thiophene (4) (12 g, 44.45 mmol, 1 eq) as solution in THF (50 mL) was added at −78° C. The resulting mixture was stirred at −78° C. for 2 h, and then allowed to gradually warm up to 25° C. and stirred for 13 hr. The mixture was then diluted with aq. sat.NH₄Cl (100 mL) and extracted with EtOAc (100 mL*2). The combined organic layers were washed with aq. sat.NaCl (100 mL), dried over anhydrous Na₂SO₄, filtered and concentrated under reduced pressure. The residue was purified by flash silica gel chromatography (ISCO®; 80 g SepaFlash® Silica Flash Column, eluent 0~7% Ethyl acetate/Petroleum ether gradient @40 mL/min). Compound methyl 3-(2-bromo-4-methyl-3-thienyl)-2-[tert-butoxycarbonyl(methyl)amino]propanoate (6) (10.2 g, 26.00 mmol, 58.50% yield) was obtained as a colorless oil. ¹H NMR (400 MHz, CHLOROFORM-d) δ=6.91-6.86 (m, 1H), 4.60-4.37 (m, 1H), 3.76 (br d, J=5.8 Hz, 3H), 3.21-3.15 (m, 1H), 3.12-2.90 (m, 1H), 2.83-2.69 (m, 3H), 2.27-2.17 (m, 3H), 1.47-1.33 (m, 9H). LCMS: m/z [M-Boc+H]⁺=293.9.

### Procedure for preparation of tert-butyl N-[1-[(2-bromo-4-methyl-3-thienyl)methyl]-2-hydroxy-ethyl]-N-methyl-carbamate (7)

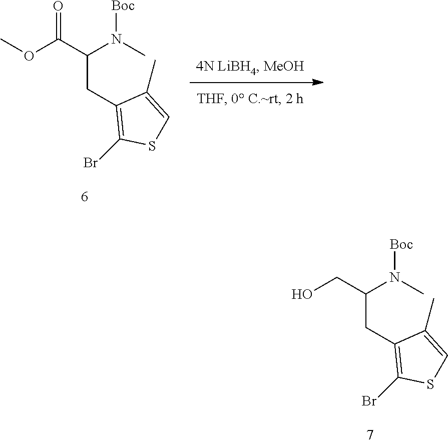 [SMILES: COC(=O)C(Cc1c(C)csc1Br)N(C)C(=O)OC(C)(C)C ; Cc1csc(Br)c1CC(CO)N(C)C(=O)OC(C)(C)C]

To a solution of methyl 3-(2-bromo-4-methyl-3-thienyl)-2-[tert-butoxycarbonyl(methyl)amino]propanoate (6) (10.2 g, 26.00 mmol, 1 eq) in THF (100 mL) under nitrogen at 0° C. was added LiBH₄ (4 M, 26.00 mL, 4 eq) and then MeOH (5 mL) at 0° C. The mixture was allowed to naturally rise to 25° C. and stirred for 2 hr under N₂. The mixture was then diluted with aq. sat.NH₄Cl (200 mL) and extracted with EtOAc (200 mL*2). The combined organic layers were washed with aq. sat.NaCl (100 mL), dried over anhydrous Na₂SO₄, filtered and concentrated under reduced pressure. The residue was purified by flash silica gel chromatography (ISCO®; 80 g SepaFlash® Silica Flash Column, eluent 0~30% Ethyl acetate/Petroleum ether gradient @40 mL/min). Compound tert-butyl N-[1-[(2-bromo-4-methyl-3-thienyl)methyl]-2-hydroxy-ethyl]-N-methyl-carbamate (7) (7.5 g, 20.59 mmol, 79.18% yield) was obtained as a colorless oil. ¹H NMR (400 MHz, CHLOROFORM-d) δ=6.81 (s, 1H), 4.29-4.11 (m, 1H), 3.75-3.62 (m, 2H), 2.81-2.59 (m, 6H), 2.16 (br s, 3H), 1.36 (br s, 9H). LCMS: m/z [M-tBu+H]⁺=309.9.

### Procedure for preparation of tert-butyl N-methyl-N-(5-methyl-3,4-dihydro-2H-thieno[2,3-b]pyran-3-yl)carbamate (8)

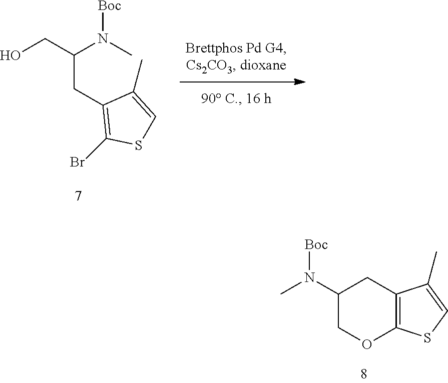 [SMILES: Cc1csc(Br)c1CC(CO)N(C)C(=O)OC(C)(C)C ; Cc1csc2c1CC(N(C)C(=O)OC(C)(C)C)CO2]

A mixture of tert-butyl N-[1-[(2-bromo-4-methyl-3-thienyl)methyl]-2-hydroxy-ethyl]-N-methyl-carbamate (7) (2 g, 5.49 mmol, 1 eq), Brettphos Pd G4 (505.37 mg, 549.00 μmol, 0.1 eq) and Cs₂CO₃ (5.37 g, 16.47 mmol, 3 eq) in dioxane (200 mL), was degassed, and stirred at 90° C. for 16 hr under N₂ atmosphere. The reaction mixture was diluted with H₂O (100 mL) and extracted with EtOAc (200 mL*2). The combined organic layers were washed with aq. sat. NaCl (100 mL), dried over anhydrous Na₂SO₄, filtered and concentrated under reduced pressure. The residue was purified by flash silica gel chromatography (ISCO®; 80 g SepaFlash® Silica Flash Column, eluent 0-7% Ethyl acetate/Petroleum ether gradient @40 mL/min). Compound tert-butyl N-methyl-N-(5-methyl-3,4-dihydro-2H-thieno[2,3-b]pyran-3-yl)carbamate (8) (600 mg, 2.12 mmol, 38.57% yield) was obtained as a yellow solid. ¹H NMR (400 MHz, CHLOROFORM-d) δ=6.13 (s, 1H), 4.64-4.35 (m, 1H), 4.24-4.03 (m, 2H), 2.82 (s, 3H), 2.74-2.58 (m, 2H), 2.07 (s, 3H), 1.49 (s, 9H). LCMS: m/z [M-tBu+H]⁺=228.0.

### Procedure for preparation of N, 5-dimethyl-3,4-dihydro-2H-thieno[2,3-b]pyran-3-amine hydrochloride (308)

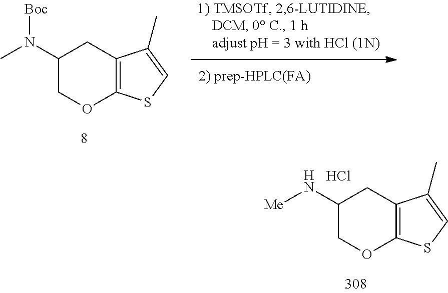 [SMILES: Cc1csc2c1CC(N(C)C(=O)OC(C)(C)C)CO2 ; CNC1COc2scc(C)c2C1 ; Cl]

To a solution of tert-butyl N-methyl-N-(5-methyl-3,4-dihydro-2H-thieno[2,3-b]pyran-3-yl)carbamate (8) (20 mg, 70.58 μmol, 1 eq) in DCM (1 mL) was added 2,6-lutidine (37.81 mg, 352.88 μmol, 41.10 μL, 5 eq) at 0° C. The resulting mixture was stirred at 0° C. for 0.5 h. Then TMSOTf (62.74 mg, 282.30 μmol, 51.01 μL, 4 eq) was added and the resulting mixture was stirred for 0.5 h. The reaction mixture was then quenched with H₂O (2 mL), its pH was adjusted to 3-4 with HCl (1N aq.) and then concentrated under vacuum at 25° C. The residue was purified by prep-HPLC (column: C18 150×30 mm; mobile phase: [water(FA)-ACN]; gradient:0%-30% B over 7 min). The product was isolated as the HCl salt. Compound N, 5-dimethyl-3,4-dihydro-2H-thieno[2,3-b]pyran-3-amine hydrochloride (308) (10.06 mg, 45.78 μmol, 64.87% yield) was isolated as an off-white solid. ¹H NMR (400 MHz, DMSO-d6) δ=8.64 (br d, J=0.9 Hz, 2H), 6.41 (d, J=1.1 Hz, 1H), 4.40 (br dd, J=4.1, 11.6 Hz, 1H), 4.30-4.20 (m, 1H), 3.75 (br d, J=2.4 Hz, 1H), 2.91 (dd, J=6.0, 16.9 Hz, 1H), 2.72-2.58 (m, 4H), 2.04 (d, J=1.1 Hz, 3H). LCMS: m/z [M+H]⁺=184.0

### Synthetic scheme for preparation of N-methyl-3-(trifluoromethyl)-6,7-dihydro-5H-thieno[3,2-b]pyran-6-amine hydrochloride enantiomer 1 (309) and enantiomer 2 (310)

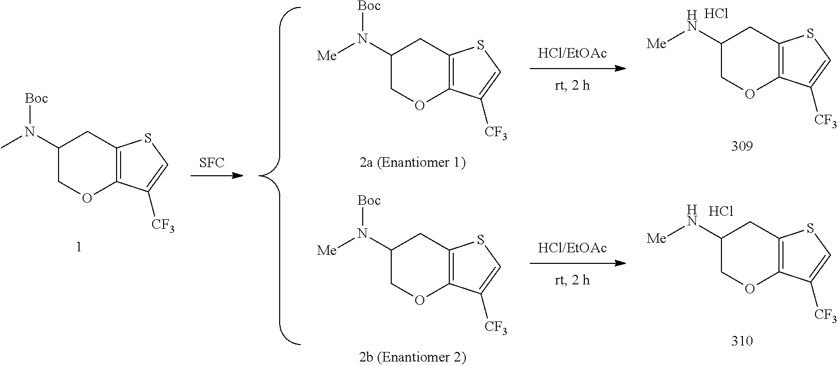

### Procedure for preparation of tert-butyl N-methyl-N-[3-(trifluoromethyl)-6,7-dihydro-5H-thieno[3,2-b]pyran-6-yl]carbamate enantiomer 1 (1a) and enantiomer 2 (1b)

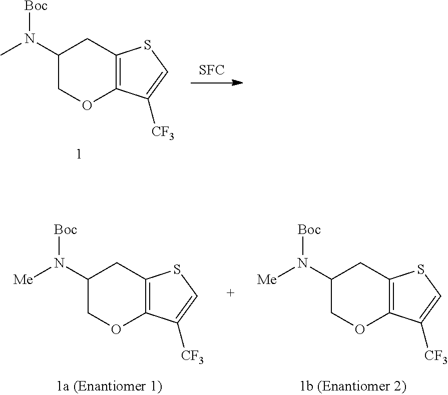

Racemic tert-butyl N-methyl-N-[3-(trifluoromethyl)-6,7-dihydro-5H-thieno[3,2-b]pyran-6-yl]carbamate (1) (120 mg) was separated by SFC (column: DAICEL CHIRALPAK AD (250 mm*50 mm, 10 μm); mobile phase: [CO₂-EtOH (0.1% NH₃H₂O)]; B %:11%, isocratic elution mode) to give enantiomers. tert-Butyl N-methyl-N-[3-(trifluoromethyl)-6,7-dihydro-5H-thieno[3,2-b]pyran-6-yl]carbamate enantiomer 1 (1a) (50 mg, 148.21 μmol, 41.67% yield) and tert-butyl N-methyl-N-[3-(trifluoromethyl)-6,7-dihydro-5H-thieno[3,2-b]pyran-6-yl]carbamate enantiomer 2 (1b) (50 mg, 148.21 μmol, 41.67% yield) both obtained as yellow oils.

### Procedure for preparation of N-methyl-3-(trifluoromethyl)-6,7-dihydro-5H-thieno[3,2-b]pyran-6-amine hydrochloride enantiomer 1 (309)

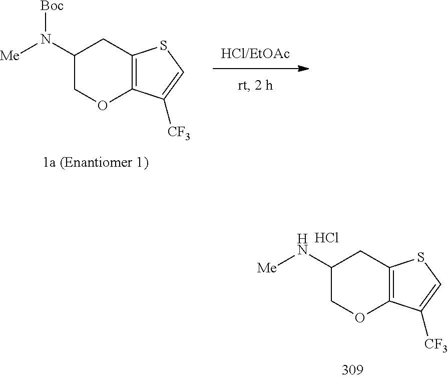 [SMILES: CN(C(=O)OC(C)(C)C)C1COc2c(C(F)(F)F)csc2C1 ; Cl ; CNC1COc2c(C(F)(F)F)csc2C1]

To a solution of tert-butyl N-methyl-N-[3-(trifluoromethyl)-6,7-dihydro-5H-thieno[3,2-b]pyran-6-yl]carbamate enantiomer 1 (1a) (50 mg, 148.21 μmol, 1 eq) in EtOAc (1 mL) was added HCl/EtOAc (2 M, 2 mL, 26.99 eq). The mixture was stirred at 25° C. for 2 hr and then concentrated. The residue was purified by prep-HPLC (column: Phenomenex luna C18 150*25 mm*10 μm; mobile phase: [water(HCl)-ACN]; gradient:1%-25% B over 10 min) to afford N-methyl-3-(trifluoromethyl)-6,7-dihydro-5H-thieno[3,2-b]pyran-6-amine hydrochloride enantiomer 1 (309) (21.28 mg, 76.97 μmol, 51.93% yield) as a white solid. ¹H NMR (400 MHz, METHANOL-d4) δ=7.82 (s, 1H), 4.59 (br d, J=12.3 Hz, 1H), 4.31 (br d, J=12.5 Hz, 1H), 3.88 (br s, 1H), 3.36 (br dd, J=5.7, 17.6 Hz, 1H), 3.09 (br d, J=17.4 Hz, 1H), 2.83 (s, 3H).LC-MS: m/z [M+H]⁺=238.0

### Procedure for preparation of N-methyl-3-(trifluoromethyl)-6,7-dihydro-5H-thieno[3,2-b]pyran-6-amine hydrochloride (310)

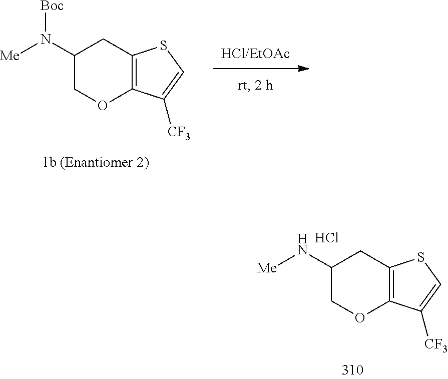 [SMILES: CN(C(=O)OC(C)(C)C)C1COc2c(C(F)(F)F)csc2C1 ; Cl ; CNC1COc2c(C(F)(F)F)csc2C1]

To a solution of tert-butyl N-methyl-N-[3-(trifluoromethyl)-6,7-dihydro-5H-thieno[3,2-b]pyran-6-yl]carbamate enantiomer 2 (1b) (50 mg, 148.21 μmol, 1 eq) in EtOAc (1 mL) was added HCl/EtOAc (2 M, 2 mL, 26.99 eq). The mixture was stirred at 25° C. for 2 hr and then concentrated. The residue was purified by prep-HPLC (column: Phenomenex luna C18 150*25 mm*10 μm; mobile phase: [water(HCl)-ACN]; gradient:1%-25% B over 10 min) to afford N-methyl-3-(trifluoromethyl)-6,7-dihydro-5H-thieno[3,2-b]pyran-6-amine hydrochloride enantiomer 2 (310) (21.99 mg, 79.54 μmol, 53.67% yield) as a white solid. ¹H NMR (400 MHz, METHANOL-d4) δ=7.82 (s, 1H), 4.59 (br d, J=12.6 Hz, 1H), 4.31 (br d, J=12.5 Hz, 1H), 3.88 (br s, 1H), 3.36 (br dd, J=5.6, 17.8 Hz, 1H), 3.09 (br d, J=17.9 Hz, 1H), 2.83 (s, 3H). LC-MS: m/z [M+H]⁺=237.9

### Synthetic scheme for preparation of 5,6-dihydro-4H-cyclopenta[b]thiophen-5-amine hydrochloride (311)

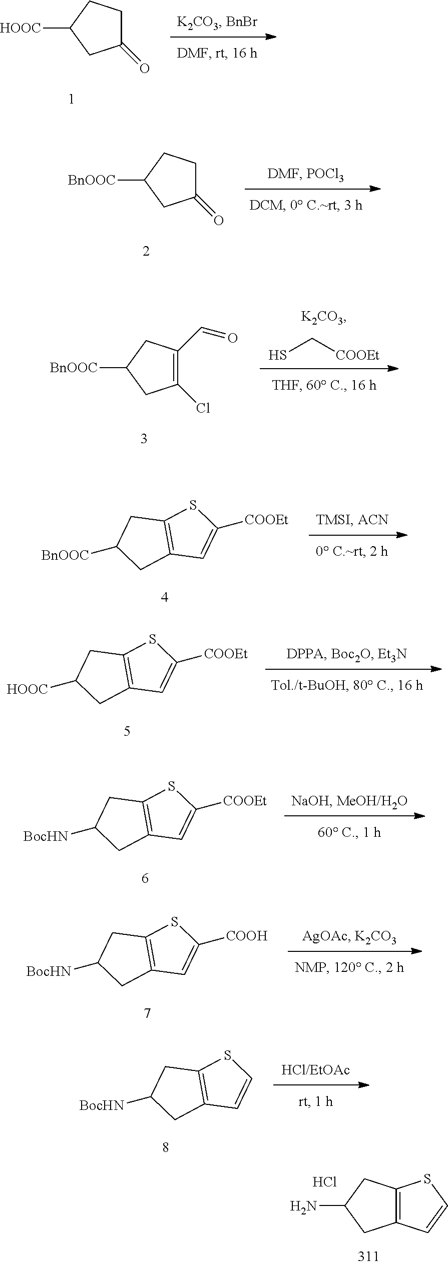

### Procedure for preparation of benzyl 3-oxocyclopentanecarboxylate (2)

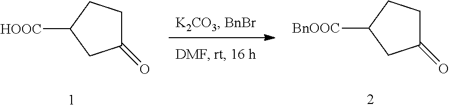

To a solution of 3-oxocyclopentanecarboxylic acid (1) (50 g, 390.24 mmol, 1 eq) in DMF (300 mL) was added K₂CO₃ (107.8 g, 780.48 mmol, 2 eq) and bromomethylbenzene (80 g, 468.29 mmol, 55.62 mL, 1.2 eq). The mixture was stirred at 25° C. for 16 hrs then filtered and the filtrate was diluted with H₂O 600 mL and extracted with EtOAc (100 mL*3). The combined organic layers were washed with brine (100 mL), dried over anhydrous Na₂SO₄, filtered and concentrated under reduced pressure to give benzyl 3-oxocyclopentanecarboxylate (2) (80 g, crude) as a yellow oil. ¹H NMR (400 MHz, CHLOROFORM-d) δ=7.45-7.30 (m, 5H), 5.17 (s, 2H), 3.26-3.12 (m, 1H), 2.59-2.43 (m, 2H), 2.43-2.28 (m, 2H), 2.27-2.10 (m, 2H).

### Procedure for preparation of 3-chloro-4-formyl-cyclopent-3-ene-1-carboxylate (3)

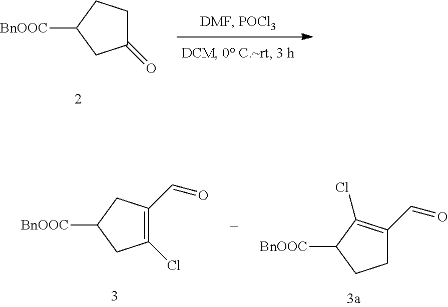

To a solution of DMF (160.7 g, 2.20 μmol, 169.22 mL, 6 eq) in DCM (600 mL) was added POCl₃ (281.0 g, 1.83 μmol, 170.84 mL, 5 eq) dropwise at 0° C. The resulting mixture was stirred at 25° C. for 1 h. To this mixture it was added a solution of benzyl 3-oxocyclopentanecarboxylate (2) (80 g, 366.56 mmol, 1 eq) in DCM (100 mL) dropwise at 0° C. The mixture was slowly warmed up to rt, stirred for 2 hrs and then quenched by addition of aq. sat.NaHCO₃ (2 L) at 0° C. The mixture extracted with EtOAc (500 mL*2). The combined organic layers were washed with brine (500 mL), dried over anhydrous Na₂SO₄, filtered and concentrated under reduced pressure to give a residue. The residue was purified by column chromatography (SiO₂, Petroleum ether/Ethyl acetate=5/1) to give benzyl 3-chloro-4-formyl-cyclopent-3-ene-1-carboxylate (3), that contained ~30% of benzyl 2-chloro-3-formylcyclopent-2-ene-1-carboxylate (3a) as impurity (55 g, 207.78 mmol, 56.68% yield) as a brown oil.

### Procedure for preparation of 5-benzyl 2-ethyl 5,6-dihydro-4H-cyclopenta[b]thiophene-2,5-dicarboxylate (4)

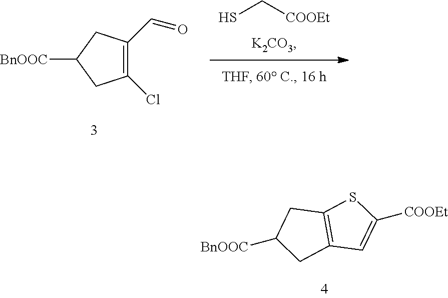

To a solution of benzyl 3-chloro-4-formyl-cyclopent-3-ene-1-carboxylate (3) from the previous step (40 g, 151.11 mmol, 1 eq) in THF (400 mL) was added K₂CO₃ (31.33 g, 226.67 mmol, 1.5 eq) and ethyl 2-sulfanylacetate (23.5 g, 195.06 mmol, 21.39 mL, 1.3 eq). The mixture was stirred at 60° C. for 16 hrs. The reaction mixture was then cooled and concentrated under reduced pressure. The residue was diluted with H₂O (200 mL) and extracted with EtOAc (200 mL*2). The combined organic layers were washed with brine (200 mL), dried over anhydrous Na₂SO₄, filtered and concentrated under reduced pressure to give a residue. The residue was purified by flash silica gel chromatography (ISCO®; 220 g SepaFlash® Silica Flash Column, eluent 0~15% Ethyl acetate/Petroleum ether gradient @60 mL/min) to give 5-benzyl 2-ethyl 5,6-dihydro-4H-cyclopenta[b]thiophene-2,5-dicarboxylate (4) (19.4 g, 42.86 mmol, 28.37% yield, 73% purity) as a yellow oil. LCMS: m/z [M+H]⁺=331.0.

### Procedure for preparation of 2-ethoxycarbonyl-5,6-dihydro-4H-cyclopenta[b]thiophene-5-carboxylic acid (5)

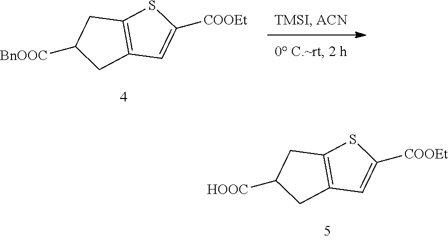

To a solution of 5-benzyl 2-ethyl 5,6-dihydro-4H-cyclopenta[b]thiophene-2,5-dicarboxylate (4) from the previous step) (10 g, 30.27 mmol, 1 eq) in CH₃CN (120 mL) was added TMSI (18.2 g, 90.80 mmol, 12.36 mL, 3 eq) at 0° C. The mixture was stirred at 25° C. for 2 hrs, and concentrated under reduced pressure. The residue was purified by flash silica gel chromatography (ISCO®; 24 g SepaFlash® Silica Flash Column, eluent 0-25% Ethyl acetate/Petroleum ether gradient @30 mL/min) to give 2-ethoxycarbonyl-5,6-dihydro-4H-cyclopenta[b]thiophene-5-carboxylic acid (5) (with ~30% regioisomer impurity) (4.4 g, 18.31 mmol, 60.50% yield) as a yellow solid. ¹H NMR (400 MHz, CHLOROFORM-d) δ=7.52 (s, 1H), 4.33 (q, J=7.2 Hz, 2H), 3.84-3.72 (m, 1H), 3.38-3.21 (m, 2H), 3.16-3.10 (m, 2H), 1.42-1.31 (t, 3H). LCMS: m/z [M+H]⁺=241.0.

### Procedure for preparation of ethyl 5-(tert-butoxycarbonylamino)-5,6-dihydro-4H-cyclopenta[b]thiophene-2 carboxylate (6)

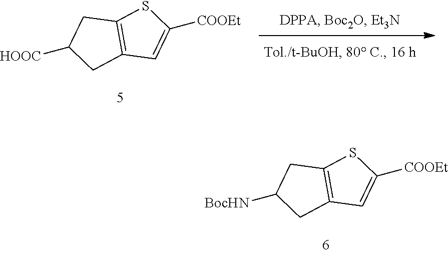 [SMILES: CCOC(=O)c1cc2c(s1)CC(C(=O)O)C2 ; CCOC(=O)c1cc2c(s1)CC(NC(=O)OC(C)(C)C)C2]

To a solution of 2-ethoxycarbonyl-5,6-dihydro-4H-cyclopenta[b]thiophene-5-carboxylic acid (5) from the previous step (4.4 g, 18.31 mmol, 1 eq) in Tol. (60 mL) and t-BuOH (15 mL) was added DPPA (7.5 g, 27.47 mmol, 5.93 mL, 1.5 eq), Et₃N (3.7 g, 36.62 mmol, 5.10 mL, 2 eq) and Boc₂O (8.0 g, 36.62 mmol, 8.41 mL, 2 eq). The mixture was stirred at 80° C. for 16 hrs under N₂ then cooled, quenched with aq. sat.NaHCO₃ (60 mL), and extracted with EtOAc (50 mL*3). The combined organic layers were washed with brine (80 mL), dried over anhydrous Na₂SO₄, filtered and concentrated under reduced pressure. The residue was purified by flash silica gel chromatography (ISCO®; 40 g SepaFlash® Silica Flash Column, eluent 0~15% Ethyl acetate/Petroleum ether gradient @40 mL/min) to give ethyl 5-(tert-butoxycarbonylamino)-5,6-dihydro-4H-cyclopenta[b]thiophene-2 carboxylate (6), (3.1 g, 9.96 mmol, 54.36% yield) as a yellow solid. ¹H NMR (400 MHz, CHLOROFORM-d) δ=7.53 (s, 1H), 4.99-4.66 (m, 2H), 4.33 (q, J=7.1 Hz, 2H), 3.35 (dd, J=7.3, 16.6 Hz, 1H), 3.17 (dd, J=7.5, 15.5 Hz, 1H), 2.79 (dd, J=5.0, 16.7 Hz, 1H), 2.61 (dd, J=4.9, 15.4 Hz, 1H), 1.46 (s, 9H), 1.36 (t, J=7.1 Hz, 3H). LCMS: m/z [M-tBu+H]⁺=256.0.

### Procedure for preparation of 5-(tert-butoxycarbonylamino)-5,6-dihydro-4H-cyclopenta[b]thiophene-2-carboxylic acid (7)

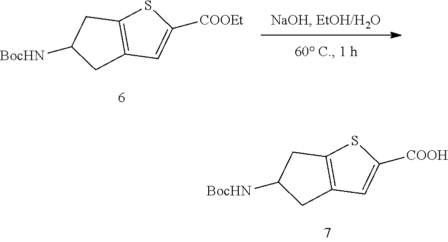 [SMILES: CCOC(=O)c1cc2c(s1)CC(NC(=O)OC(C)(C)C)C2 ; CC(C)(C)OC(=O)NC1Cc2cc(C(=O)O)sc2C1]

To a solution of ethyl 5-(tert-butoxycarbonylamino)-5,6-dihydro-4H-cyclopenta[b]thiophene-2 carboxylate (6) (1 g, 3.21 mmol, 1 eq) in EtOH (10 mL) and H₂O (2 mL) was added NaOH (385.33 mg, 9.63 mmol, 3 eq). The mixture was stirred at 60° C. for 1 hr. The reaction mixture was then cooled and concentrated under reduced pressure to remove EtOH. and diluted with H₂O (50 mL). HCl (1N aq.) was added to pH=6. The solid formed was filtered and dried under reduced pressure to give 5-(tert-butoxycarbonylamino)-5,6-dihydro-4H-cyclopenta[b]thiophene-2-carboxylic acid (7) (900 mg, crude) as a white solid.

### Procedure for preparation of tert-butyl N-(5,6-dihydro-4H-cyclopenta[b]thiophen-5-yl)carbamate (8)

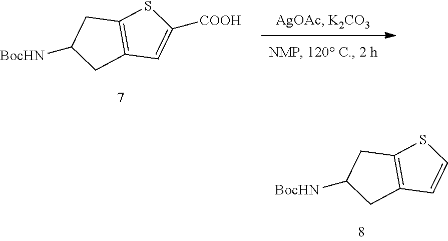 [SMILES: CC(C)(C)OC(=O)NC1Cc2cc(C(=O)O)sc2C1 ; CC(C)(C)OC(=O)NC1Cc2ccsc2C1]

To a solution of 5-(tert-butoxycarbonylamino)-5,6-dihydro-4H-cyclopenta[b]thiophene-2-carboxylic acid (7) (900 mg, 3.18 mmol, 1 eq) in NMP (10 mL) was added AgOAc (53.02 mg, 317.64 μmol, 0.1 eq) and K₂CO₃ (1.32 g, 9.53 mmol, 3 eq). The mixture was stirred at 120° C. for 2 hr under N₂. Then the reaction was cooled to rt and diluted with H₂O (50 mL) and extracted with EtOAc (30 mL*2). The combined organic layers were washed with brine (30 mL), dried over anhydrous Na₂SO₄, filtered and concentrated under reduced pressure. The residue was purified by flash silica gel chromatography (ISCO®; 20 g SepaFlash® Silica Flash Column, eluent 0~10% Ethyl acetate/Petroleum ether gradient @30 mL/min) to give tert-butyl N-(5,6-dihydro-4H-cyclopenta[b]thiophen-5-yl)carbamate (8) (230 mg, 951.39 μmol, 29.95% yield) as a white solid. ¹H NMR (400 MHz, CHLOROFORM-d) δ=7.18 (d, J=4.9 Hz, 1H), 6.81 (d, J=4.9 Hz, 1H), 4.98-4.75 (m, 2H), 3.32 (br dd, J=7.3, 15.9 Hz, 1H), 3.17 (dd, J=7.3, 15.3 Hz, 1H), 2.74 (dd, J=4.3, 15.8 Hz, 1H), 2.58 (dd, J=4.7, 15.3 Hz, 1H), 1.47 (s, 9H). LCMS: m/z [M-tBu+H]⁺=184.0, m/z [M-NHBoc]⁺=123.0

### Procedure for preparation of 5,6-dihydro-4H-cyclopenta[b]thiophen-5-amine hydrochloride (311)

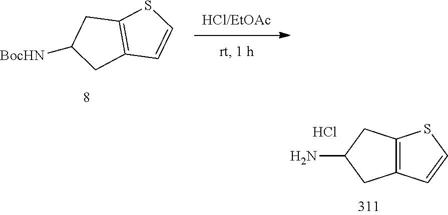 [SMILES: CC(C)(C)OC(=O)NC1Cc2ccsc2C1 ; NC1Cc2ccsc2C1 ; Cl]

A solution of tert-butyl N-(5,6-dihydro-4H-cyclopenta[b]thiophen-5-yl)carbamate (8) (70 mg, 292.48 μmol, 1 eq) in HCl/EtOAc (2 mL) was stirred at 25° C. for 1 hr. The reaction mixture was concentrated under reduced pressure. The residue was triturated with EtOAc 2 mL at 25° C. for 5 min, filtered and dried under reduced pressure to give 5,6-dihydro-4H-cyclopenta[b]thiophen-5-amine hydrochloride (311) (41 mg, 291.56 μmol, 99.68% yield) as a white solid. ¹H NMR (400 MHz, METHANOL-d4) δ=7.34 (d, J=5.0 Hz, 1H), 6.87 (d, J=5.0 Hz, 1H), 4.41 (tt, J=3.9, 7.6 Hz, 1H), 3.42 (dd, J=7.6, 16.4 Hz, 1H), 3.30-3.22 (m, 1H), 2.98 (br dd, J=2.8, 16.4 Hz, 1H), 2.83 (br dd, J=3.4, 16.1 Hz, 1H). LCMS: m/z [M+H]⁺=140.0.

### Synthetic scheme for synthesis of 6-amino-4,5,6,7-tetrahydro-2-benzothiophene-1-carbonitrile hydrochloride enantiomer 1 (312) and enantiomer 2 (313)

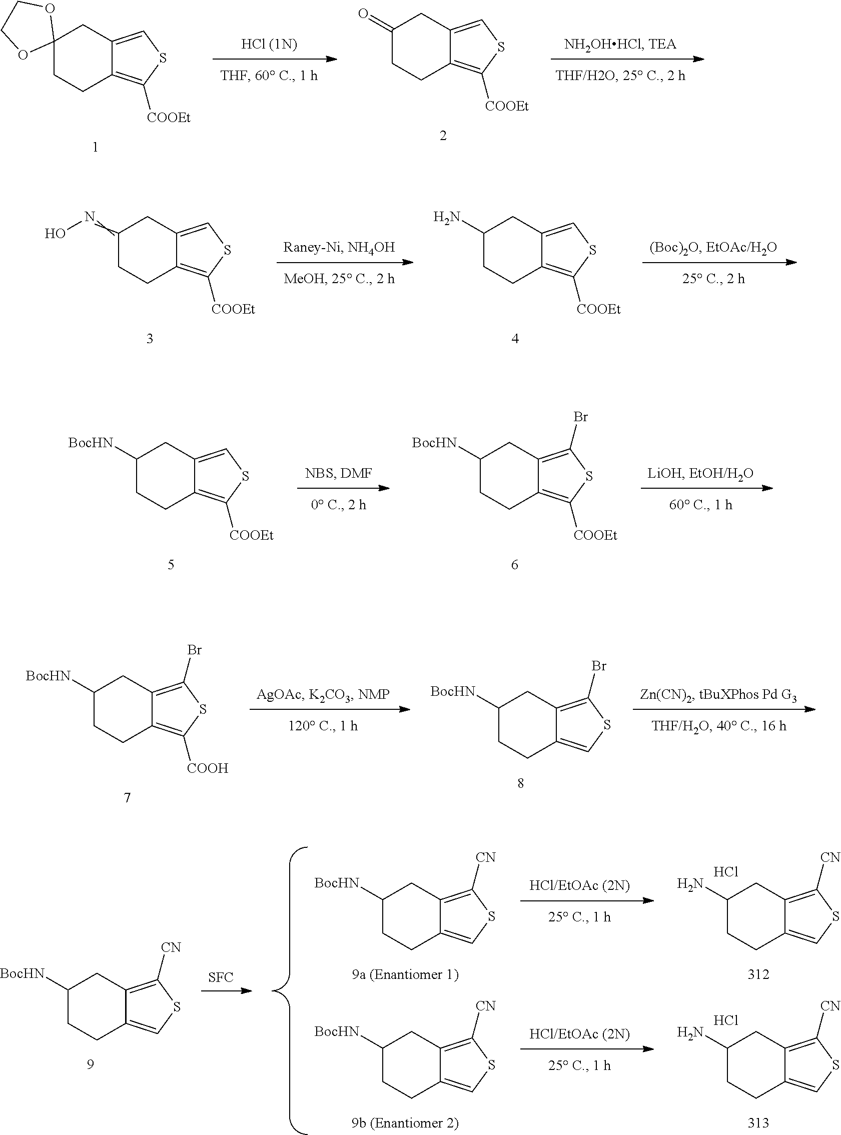

### Procedure for preparation of ethyl 5-oxo-6,7-dihydro-4H-2-benzothiophene-1-carboxylate (5)

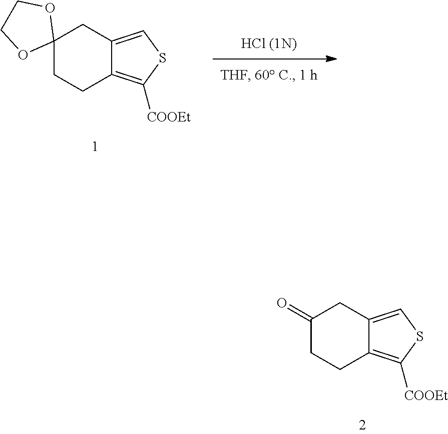 [SMILES: CCOC(=O)c1scc2c1CCC1(C2)OCCO1 ; CCOC(=O)c1scc2c1CCC(=O)C2]

To a solution of ethyl spiro[1,3-dioxolane-2,5′-6,7-dihydro-4H-2-benzothiophene]-1′-carboxylate (1) (14 g, 52.17 mmol, 1 eq) was added 1N aq HCl (200 mL) and THF (200 mL). The mixture was stirred at 60° C. for 1 hr. The reaction was then quenched with aq sat. NaHCO₃ (100 mL) at 25° C. and the resulting mixture was extracted with EtOAc 100 mL (50 mL*2). The combined organic layers were washed with NaCl aq. sat. (50 mL*2), dried over Na₂SO₄, filtered and concentrated under reduced pressure. The residue was purified by flash silica gel chromatography (ISCO®; 80 g SepaFlash® Silica Flash Column, eluent 0-25% Ethyl acetate/Petroleum ether gradient @60 mL/min). Compound ethyl 5-oxo-6,7-dihydro-4H-2-benzothiophene-1-carboxylate (2) (9.1 g, 40.58 mmol, 77.77% yield) was obtained as a yellow oil. LCMS: m/z [M+H]⁺=225.0

### Procedure for preparation of ethyl 5-hydroxyimino-6,7-dihydro-4H-2-benzothiophene-1-carboxylate (3)

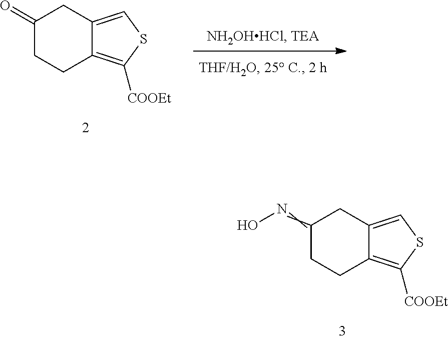 [SMILES: CCOC(=O)c1scc2c1CCC(=O)C2 ; CCOC(=O)c1scc2c1CCC(=NO)C2]

To a solution of ethyl 5-oxo-6,7-dihydro-4H-2-benzothiophene-1-carboxylate (2) (9.1 g, 40.58 mmol, 1 eq) in THF (50 mL) was added TEA (10.26 g, 101.44 mmol, 14.12 mL, 2.5 eq) and NH₂OH·HCl (5.64 g, 81.15 mmol, 2 eq). The mixture was stirred at 25° C. for 2 hr then diluted with H₂O (100 mL), and extracted with EtOAc (100 mL*2). The combined organic layers were washed with aq. sat. NaCl (50 mL*2), dried over Na₂SO₄, filtered and concentrated under reduced pressure. The residue was purified by flash silica gel chromatography (ISCO®; 80 g SepaFlash® Silica Flash Column, eluent 0~50% Ethyl acetate/Petroleum ether gradient @60 mL/min). Compound ethyl 5-hydroxyimino-6,7-dihydro-4H-2-benzothiophene-1-carboxylate (3) (8 g, 33.43 mmol, 82.40% yield) was obtained as a yellow solid. LCMS: m/z [M+H]⁺=240.0

### Procedure for preparation of ethyl 5-amino-4,5,6,7-tetrahydro-2-benzothiophene-1-carboxylate (4)

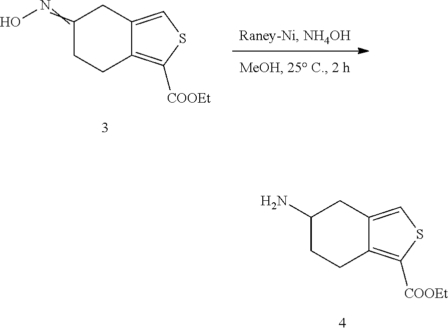 [SMILES: CCOC(=O)c1scc2c1CCC(=NO)C2 ; CCOC(=O)c1scc2c1CCC(N)C2]

To a solution of ethyl 5-hydroxyimino-6,7-dihydro-4H-2-benzothiophene-1-carboxylate (3) (7.9 g, 33.01 mmol, 1 eq) in MeOH (50 mL) was added Raney-Ni (2.83 g, 33.01 mmol, 1 eq) and NH₃·H₂O (34%, 340.30 mg, 3.30 mmol, 373.95 μL, 0.1 eq). The mixture was stirred at 25° C. for 2 hr under H₂ (15 psi). The reaction mixture was then filtered and concentrated under reduced pressure to remove MeOH. The crude product ethyl 5-amino-4,5,6,7-tetrahydro-2-benzothiophene-1-carboxylate (4) (7 g, 31.07 mmol, 94.11% yield) was obtained as a yellow solid and used into the next step without further purification. LCMS: m/z [M+H]⁺=226.0

### Procedure for preparation of ethyl 5-(tert-butoxycarbonylamino)-4,5,6,7-tetrahydro-2-benzothiophene-1-carboxylate (5)

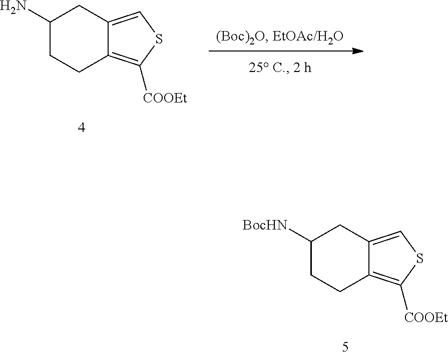 [SMILES: CCOC(=O)c1scc2c1CCC(N)C2 ; CCOC(=O)c1scc2c1CCC(NC(=O)OC(C)(C)C)C2]

To a solution of ethyl 5-amino-4,5,6,7-tetrahydro-2-benzothiophene-1-carboxylate (4) (7 g, 31.07 mmol, 1 eq) in EtOAc (50 mL) and H₂O (50 mL) was added aq. sat. sodium;hydrogen carbonate (13.05 g, 155.34 mmol, 6.04 mL, 5 eq) and tert-butoxycarbonyl tert-butyl carbonate (13.56 g, 62.14 mmol, 14.28 mL, 2 eq). The mixture was stirred at 25° C. for 2 hr. The reaction mixture was extracted with EtOAc (150 mL*2). The combined organic layers were washed with aq. sat. NaCl (50 mL*2), dried over Na₂SO₄, filtered and concentrated under reduced pressure. The residue was purified by flash silica gel chromatography (ISCO®; 80 g SepaFlash® Silica Flash Column, eluent 0~15% Ethyl acetate/Petroleum ether gradient @60 mL/min). Compound ethyl 5-(tert-butoxycarbonylamino)-4,5,6,7-tetrahydro-2-benzothiophene-1-carboxylate (5) (4 g, 12.29 mmol, 39.56% yield) was obtained as a yellow solid. LCMS: m/z [M+H-tBu]⁺=270.0

### Procedure for preparation of ethyl 3-bromo-5-(tert-butoxycarbonylamino)-4,5,6,7-tetrahydro-2-benzothiophene-1-carboxylate (6)

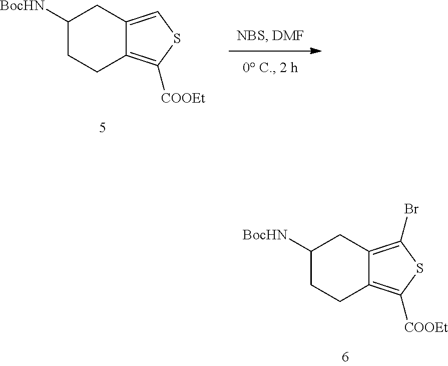 [SMILES: CCOC(=O)c1scc2c1CCC(NC(=O)OC(C)(C)C)C2 ; CCOC(=O)c1sc(Br)c2c1CCC(NC(=O)OC(C)(C)C)C2]

To a solution of ethyl 5-(tert-butoxycarbonylamino)-4,5,6,7-tetrahydro-2-benzothiophene-1-carboxylate (5) (3.9 g, 11.98 mmol, 1 eq) in DMF (40 mL) was added NBS (2.77 g, 15.58 mmol, 1.3 eq). The mixture was stirred at 0° C. for 2 hr and then quenched with aq. Na₂SO₃ (50 mL) at 25° C. and then extracted with EtOAc (50 mL*2). The combined organic layers were washed with aq. sat. NaCl (30 mL*2), dried over Na₂SO₄, filtered and concentrated under reduced pressure. The residue was purified by flash silica gel chromatography (ISCO®; 40 g SepaFlash® Silica Flash Column, eluent 0~15% Ethyl acetate/Petroleum ether gradient @60 mL/min). Compound ethyl 3-bromo-5-(tert-butoxycarbonylamino)-4,5,6,7-tetrahydro-2-benzothiophene-1-carboxylate (6) (2.5 g, 6.18 mmol, 51.59% yield) was obtained as a yellow solid. LCMS: m/z [M+H-tBu]⁺=348.0

### Procedure for preparation of 3-bromo-5-(tert-butoxycarbonylamino)-4,5,6,7-tetrahydro-2-benzothiophene-1-carboxylic acid (7)

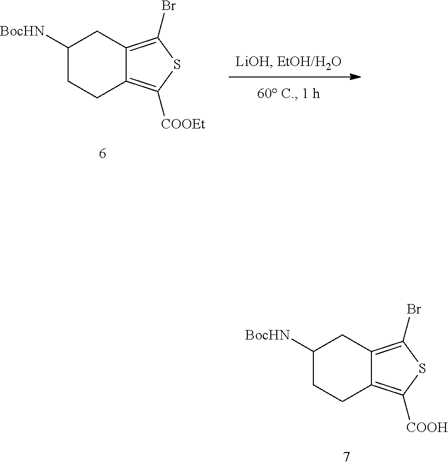 [SMILES: CCOC(=O)c1sc(Br)c2c1CCC(NC(=O)OC(C)(C)C)C2 ; CC(C)(C)OC(=O)NC1CCc2c(C(=O)O)sc(Br)c2C1]

To a solution of ethyl 3-bromo-5-(tert-butoxycarbonylamino)-4,5,6,7-tetrahydro-2-benzothiophene-1-carboxylate (6) (2.5 g, 6.18 mmol, 1 eq) in EtOH (30 mL) and H₂O (30 mL) was added LiOH·H₂O (1.04 g, 24.73 mmol, 4 eq). The mixture was stirred at 60° C. for 1 hr and then concentrated under reduced pressure to remove EtOH. To the mixture was added aq. HCl (1M, 50 ml) to pH=6. The solid formed was filtered collected and dried. Compound 3-bromo-5-(tert-butoxycarbonylamino)-4,5,6,7-tetrahydro-2-benzothiophene-1-carboxylic acid (7) (2.2 g, 5.85 mmol, 94.56% yield) was obtained as a white solid. LCMS: m/z [M+H]⁺=320.0

### Procedure for preparation of tert-butyl N-(3-bromo-4,5,6,7-tetrahydro-2-benzothiophen-5-yl)carbamate (8)

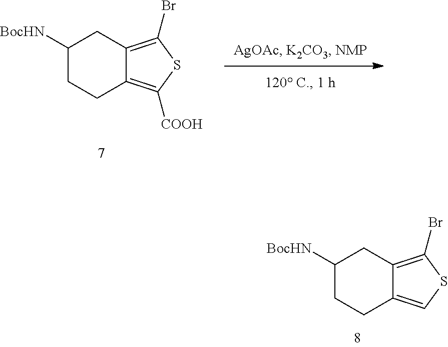

To a solution of 3-bromo-5-(tert-butoxycarbonylamino)-4,5,6,7-tetrahydro-2-benzothiophene-1-carboxylic acid (7) (2.2 g, 5.85 mmol, 1 eq) in NMP (30 mL) was added AgOAc (487.96 mg, 2.92 mmol, 0.5 eq) and K₂CO₃ (2.42 g, 17.54 mmol, 3 eq). The resulting mixture was stirred at 120° C. for 1 hr then cooled and filtered. The filtrate was diluted with H₂O (40 ml) and extracted with EtOAc (30 mL*2). The combined organic layers were washed with aq. sat. NaCl (15 mL*2), dried over Na₂SO₄, filtered and concentrated under reduced pressure. The residue was purified by flash silica gel chromatography (ISCO®; 40 g SepaFlash® Silica Flash Column, eluent 0~15% Ethyl acetate/Petroleum ether gradient @50 mL/min). Compound tert-butyl N-(3-bromo-4,5,6,7-tetrahydro-2-benzothiophen-5-yl)carbamate (8) (1.3 g, 3.91 mmol, 66.92% yield) was obtained as a white solid. LCMS: m/z [M+H-tBu]⁺=277.0

### Procedure for preparation of tert-butyl N-(3-cyano-4,5,6,7-tetrahydro-2-benzothiophen-5-yl)carbamate (9)

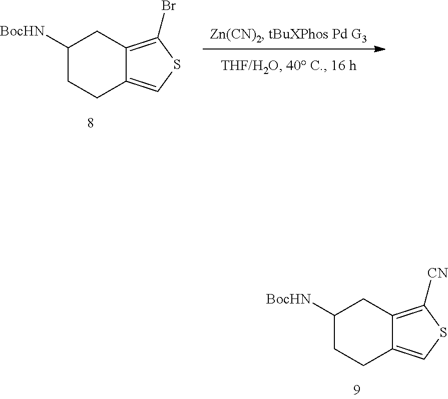 [SMILES: CC(C)(C)OC(=O)NC1CCc2csc(Br)c2C1 ; CC(C)(C)OC(=O)NC1CCc2csc(C#N)c2C1]

To a solution of tert-butyl N-(3-bromo-4,5,6,7-tetrahydro-2-benzothiophen-5-yl)carbamate (8) (500 mg, 1.50 mmol, 1 eq) in THF (5 mL) and H₂O (5 mL) was added tBuXPhos Pd G3 (119.54 mg, 150.49 μmol, 0.1 eq) and Zn(CN)₂ (229.72 mg, 1.96 mmol, 124.17 μL, 1.3 eq). The mixture was stirred at 40° C. for 16 hr then cooled to rt quenched with aq. sat. NaHCO₃ (50 mL) and extracted with EtOAc 30 mL (15 mL*2). The combined organic layers were washed with aq. sat. NaCl (10 mL*2), dried over Na₂SO₄, filtered and concentrated under reduced pressure. The residue was purified by prep-HPLC (neutral condition:column: Waters Xbridge 150*25 mm*5 μm; mobile phase: [water(NH₄HCO₃)-ACN]; gradient:44%-74% B over 9 min). Compound tert-butyl N-(3-cyano-4,5,6,7-tetrahydro-2-benzothiophen-5-yl)carbamate (9) (140 mg, 502.93 μmol, 33.42% yield) was obtained as a white solid. LCMS: m/z [M+H-tBu]⁺=224.1

### Procedure for preparation of tert-butyl N-(3-cyano-4,5,6,7-tetrahydro-2-benzothiophen-5-yl)carbamate enantiomer 1 (9a) and enantiomer 2 (9b)

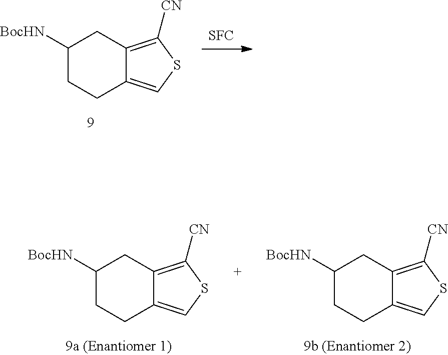

Racemic tert-butyl N-(3-cyano-4,5,6,7-tetrahydro-2-benzothiophen-5-yl)carbamate (9) (140 mg) was separated to its enantiomers by SFC (column: DAICEL CHIRALPAK IG (250 mm*50 mm, 10 μm); mobile phase: [CO₂-EtOH (0.1% NH₃H₂O)]; B %:21.5%, isocratic elution mode). Compound tert-butyl N-(3-cyano-4,5,6,7-tetrahydro-2-benzothiophen-5-yl)carbamate enantiomer 1 (9a) (70 mg, 251.46 mol, 50.00% yield) was obtained as a white solid. Compound tert-butyl N-(3-cyano-4,5,6,7-tetrahydro-2-benzothiophen-5-yl)carbamate (9b) (70 mg, 251.46 μmol, 50.00% yield) was obtained as a white solid.

### Procedure for preparation of 6-amino-4,5,6,7-tetrahydro-2-benzothiophene-1-carbonitrile hydrochloride enantiomer 1 (312)

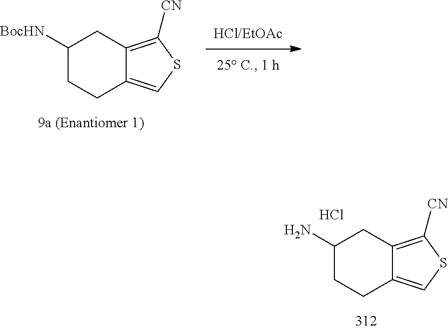 [SMILES: CC(C)(C)OC(=O)NC1CCc2csc(C#N)c2C1 ; N#Cc1scc2c1CC(N)CC2 ; Cl]

To a solution of tert-butyl N-(3-cyano-4,5,6,7-tetrahydro-2-benzothiophen-5-yl)carbamate enantiomer 1 (9a) (70 mg, 251.46 μmol, 1 eq) in EtOAc (1 mL) was added HCl/EtOAc (2 M, 2.80 mL, 22.27 eq). The mixture was stirred at 25° C. for 1 hr and then concentrated under reduced pressure to remove EtOAc. The residue was suspended in EtOAc (2 mL) at 25° C. for 10 min. Followed filtration and the filter cake was collected and dried to afford the product. Compound 6-amino-4,5,6,7-tetrahydro-2-benzothiophene-1-carbonitrile hydrochloride enantiomer 1 (312) (41.04 mg, 187.75 μmol, 74.66% yield) was obtained as a white solid. ¹H NMR (400 MHz, METHANOL-d₄) δ=7.54 (s, 1H), 3.70-3.59 (m, 1H), 3.43-3.37 (m, 1H), 3.08-2.98 (m, 1H), 2.91-2.78 (m, 2H), 2.30-2.19 (m, 1H), 1.89 (dtd, J=5.4, 11.0, 12.9 Hz, 1H). LCMS: m/z [M+H]⁺=179.0

### Procedure for preparation of 6-amino-4,5,6,7-tetrahydro-2-benzothiophene-1-carbonitrile hydrochloride enantiomer 2 (313)

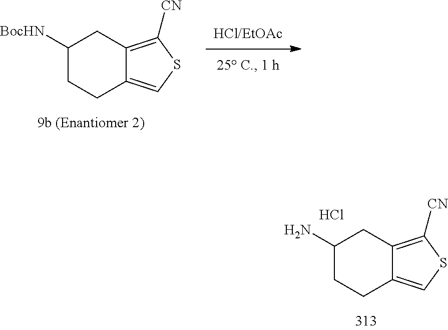 [SMILES: CC(C)(C)OC(=O)NC1CCc2csc(C#N)c2C1 ; N#Cc1scc2c1CC(N)CC2 ; Cl]

To a solution of tert-butyl N-(3-cyano-4,5,6,7-tetrahydro-2-benzothiophen-5-yl)carbamate enantiomer 2 (9b) (70 mg, 251.46 μmol, 1 eq) in EtOAc (1 mL) was added HCl/EtOAc (2 M, 2.80 mL, 22.27 eq). The mixture was stirred at 25° C. for 1 hr and then concentrated under reduced pressure. The residue was suspended in EtOAc (2 mL) at 25° C. for 10 min. The mixture was then filtred and, the filter cake was then collected and dried to afford the product. Compound 6-amino-4,5,6,7-tetrahydro-2-benzothiophene-1-carbonitrile hydrochloride enantiomer 2 (313) (38.63 mg, 175.11 μmol, 69.64% yield) was obtained as a white solid. ¹H NMR (400 MHz, METHANOL-d₄) δ=7.55-7.54 (m, 1H), 3.69-3.59 (m, 1H), 3.43-3.36 (m, 1H), 3.08-2.97 (m, 1H), 2.90-2.79 (m, 2H), 2.29-2.20 (m, 1H), 1.95 (br d, J=1.0 Hz, 1H). LCMS: m/z [M+H]⁺=179.0

### Synthetic Scheme for preparation of N-methyl-3-(trifluoromethyl)-5,6-dihydro-4H-cyclopenta[b]thiophen-5-amine hydrochloride enantiomer 1 (314) and enantiomer 2 (315)

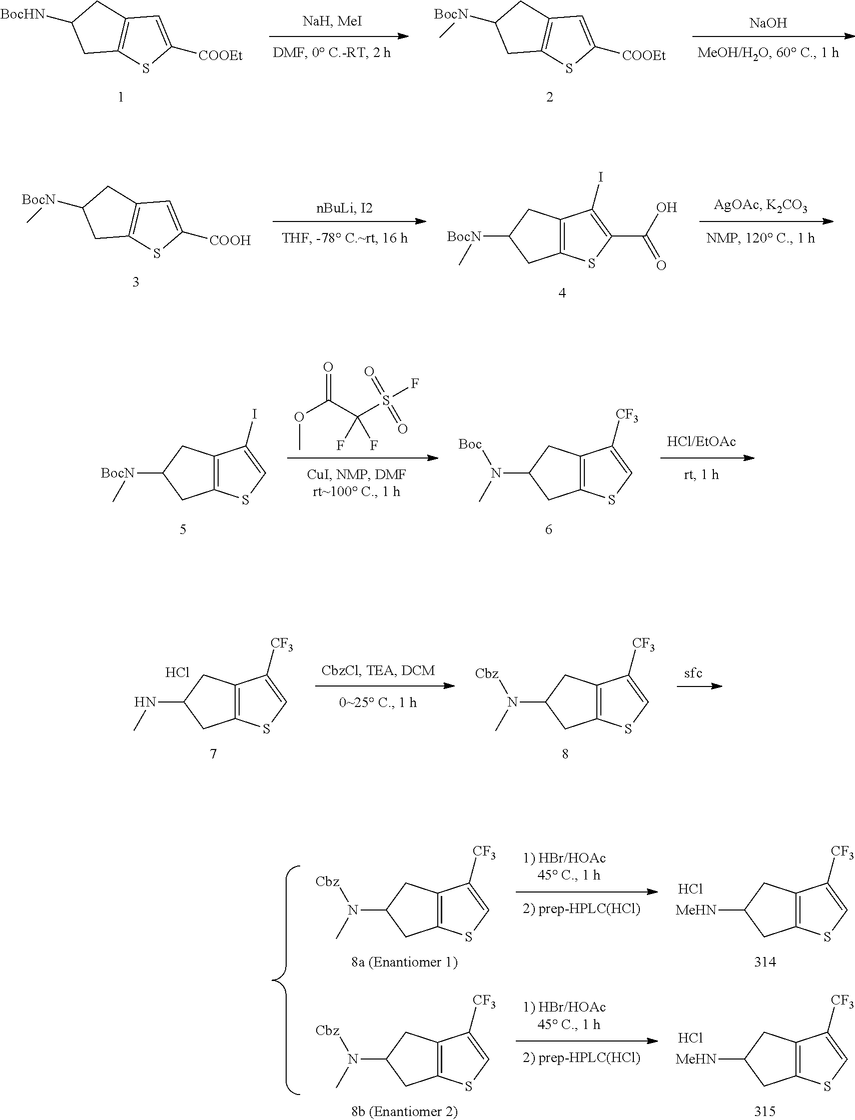

### Procedure for preparation of ethyl 5-[tert-butoxycarbonyl(methyl)amino]J-5,6-dihydro-4H-cyclopenta[b]thiophene-2-carboxylate (2)

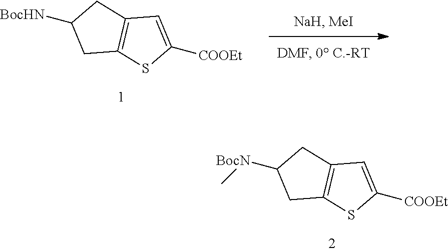 [SMILES: CCOC(=O)c1cc2c(s1)CC(NC(=O)OC(C)(C)C)C2 ; CCOC(=O)c1cc2c(s1)CC(N(C)C(=O)OC(C)(C)C)C2]

To a solution of ethyl 5-(tert-butoxycarbonylamino)-5,6-dihydro-4H-cyclopenta[b]thiophene-2-carboxylate (1) (3.3 g, 10.60 mmol, 1 eq) in DMF (40 ml) was added NaH (551.01 mg, 13.78 mmol, 60% mineral oil suspension, 1.3 eq) at 0° C. The mixture was stirred for 0.5 h, then it was added MeI (1.96 g, 13.78 mmol, 857.65 μL, 1.3 eq). The mixture was allowed to warm up to rt and stirred for 1.5 hr. The reaction mixture was then quenched by addition aq. sat.NH₄Cl (50 mL) at 0° C., and extracted with EtOAc (50 mL*2). The combined organic layers were washed with brine (50 mL), dried over anhydrous Na₂SO₄, filtered and concentrated under reduced pressure. The residue was purified by flash silica gel chromatography (ISCO®; 20 g SepaFlash® Silica Flash Column, eluent 0~10% Ethyl acetate/Petroleum ether gradient @40 mL/min). Compound ethyl 5-[tert-butoxycarbonyl(methyl)amino]-5,6-dihydro-4H-cyclopenta[b]thiophene-2-carboxylate (2) (2.6 g, 7.99 mmol, 75.39% yield) was obtained as a yellow oil. LC-MS: m/z [M-tBu+H]⁺=270.0

### Procedure for preparation of 5-[tert-butoxycarbonyl(methyl)amino]-5,6-dihydro-4H-cyclopenta[b]thiophene-2-carboxylic acid (3)

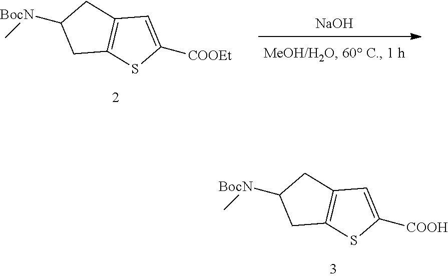 [SMILES: CCOC(=O)c1cc2c(s1)CC(N(C)C(=O)OC(C)(C)C)C2 ; "NaOH" ; CN(C(=O)OC(C)(C)C)C1Cc2cc(C(=O)O)sc2C1]

To a solution of ethyl 5-[tert-butoxycarbonyl(methyl)amino]-5,6-dihydro-4H-cyclopenta[b]thiophene-2-carboxylate (2) (2.6 g, 7.99 mmol, 1 eq) in MeOH (30 mL) and H₂O (10 mL) was added NaOH (639.12 mg, 15.98 mmol, 2 eq). The mixture was stirred at 60° C. for 1 hr. The reaction mixture was then cooled and concentrated under reduced pressure to remove MeOH and then diluted with H₂O (30 mL) and acidified to pH=6 with 1 M HCl aqueous solution. The white precipitate that was formed was collected by filtration and air-dried to afford the product. Compound 5-[tert-butoxycarbonyl(methyl)amino]-5,6-dihydro-4H-cyclopenta[b]thiophene-2-carboxylic acid (3) (2.3 g, 7.73 mmol, 96.81% yield) was obtained as a white solid. ¹H NMR (400 MHz, CHLOROFORM-d) δ=7.61 (s, 1H), 5.55-5.17 (m, 1H), 3.23 (dd, J=8.8, 17.1 Hz, 1H), 3.11-2.91 (m, 2H), 2.87-2.70 (m, 4H), 1.47 (s, 9H). LC-MS: m/z [M-tBu+H]⁺=242.0

### Procedure for preparation of 5-[tert-butoxycarbonyl(methyl)amino]-3-iodo-5,6-dihydro-4H-cyclopenta[b]thiophene-2-carboxylic acid (4)

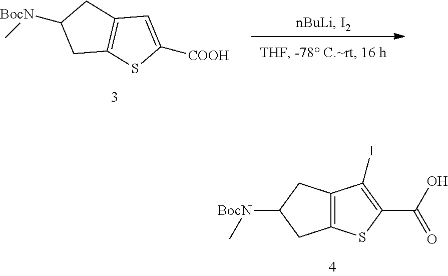 [SMILES: CN(C(=O)OC(C)(C)C)C1Cc2cc(C(=O)O)sc2C1 ; "nBuLi, I2" ; CN(C(=O)OC(C)(C)C)C1Cc2sc(C(=O)O)c(I)c2C1]

To a solution of 5-[tert-butoxycarbonyl(methyl)amino]-5,6-dihydro-4H-cyclopenta[b]thiophene-2-carboxylic acid (3) (1.8 g, 6.05 mmol, 1 eq) in THF (20 mL) was added n-BuLi (2.5 M, 6.05 mL, 2.5 eq) dropwise at −78° C. under N₂. The mixture was stirred for 1 h. Then I₂ (3.07 g, 12.11 mmol, 2.44 mL, 2 eq) in THF (10 mL) was added dropwise under N₂. The resulting mixture was stirred at −78° C. for 1 hr and at 25° C. for 14 hr and then quenched with aq. sat.NH₄Cl (50 mL) and extracted with EtOAc (50 mL*2). The combined organic layers were washed with aq. sat.NaCl (50 mL), dried over anhydrous Na₂SO₄, filtered and concentrated under reduced pressure to give a residue. The residue was purified by prep-HPLC (column: Phenomenex luna C18 150*40 mm*15 μm; mobile phase: [water(FA)-ACN]; gradient:40%-70% B over 15 min). Compound 5-[tert-butoxycarbonyl(methyl)amino]-3-iodo-5,6-dihydro-4H-cyclopenta[b]thiophene-2-carboxylic acid (4) (1.7 g, 4.02 mmol, 66.35% yield) was obtained as a yellow solid. ¹H NMR (400 MHz, CHLOROFORM-d) δ=5.54-5.14 (m, 1H), 3.37 (dd, J=8.8, 17.2 Hz, 1H), 3.16-2.96 (m, 2H), 2.80 (br s, 4H), 1.47 (s, 9H). LC-MS: m/z [M-Boc+H]⁺=323.9

### Procedure for preparation of tert-butyl N-(3-iodo-5,6-dihydro-4H-cyclopenta[b]thiophen-5-yl)-N-methyl-carbamate (5)

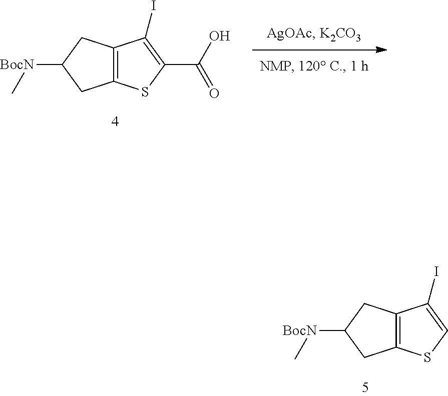

To a solution of 5-[tert-butoxycarbonyl(methyl)amino]-3-iodo-5,6-dihydro-4H-cyclopenta[b]thiophene-2-carboxylic acid (4) (1.7 g, 4.02 mmol, 1 eq) in NMP (20 mL) was added AgOAc (67.04 mg, 401.64 μmol, 0.1 eq) and K₂CO₃ (1.67 g, 12.05 mmol, 3 eq). The mixture was stirred at 120° C. for 1 hr. The reaction mixture was then cooled and diluted with H₂O (50 mL) and extracted with EtOAc (50 mL*2). The combined organic layers were washed with aq. sat.NaCl (10 mL), dried over anhydrous Na₂SO₄, filtered and concentrated under reduced pressure to give a residue. The residue was purified by flash silica gel chromatography (ISCO®; 12 g SepaFlash® Silica Flash Column, eluent 0~10% Ethyl acetate/Petroleum ether gradient @40 mL/min). Compound tert-butyl N-(3-iodo-5,6-dihydro-4H-cyclopenta[b]thiophen-5-yl)-N-methyl-carbamate (5) (1.37 g, 3.61 mmol, 89.94% yield) was obtained as a colorless oil. ¹H NMR (400 MHz, CHLOROFORM-d) δ=7.24 (s, 1H), 5.64-5.10 (m, 1H), 3.30 (br dd, J=8.9, 16.0 Hz, 1H), 3.10-2.90 (m, 2H), 2.87-2.63 (m, 4H), 1.47 (s, 9H). LC-MS: m/z [M-tBu+H]⁺=323.9

### Procedure for preparation of tert-butyl N-methyl-N-[3-(trifluoromethyl)-5,6-dihydro-4H-cyclopenta[b]thiophen-5-yl]carbamate (6)

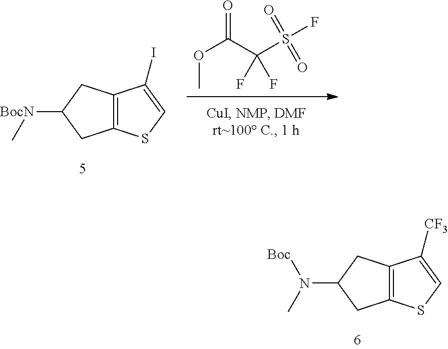

To a solution of tert-butyl N-(3-iodo-5,6-dihydro-4H-cyclopenta[b]thiophen-5-yl)-N-methyl-carbamate (5) (400 mg, 1.05 mmol, 1 eq) in DMF (10 mL) was added CuI (220.95 mg, 1.16 mmol, 1.1 eq) and NMP (522.76 mg, 5.27 mmol, 512.51 μL, 5 eq). The mixture was stirred for 10 min. Then methyl 2,2-difluoro-2-fluorosulfonyl-acetate (1.01 g, 5.27 mmol, 670.93 μL, 5 eq) was added. and the mixture was stirred at 100° C. for 1 hr. The mixture was then cooled and diluted with H₂O (50 mL) and extracted with EtOAc (30 mL*2). The combined organic layers were washed with aq. sat.NaCl (20 mL), dried over anhydrous Na₂SO₄, filtered and concentrated under reduced pressure. The residue was purified by flash silica gel chromatography (ISCO®; 12 g SepaFlash® Silica Flash Column, eluent 0~10% Ethyl acetate/Petroleum ether gradient @40 mL/min). Compound tert-butyl N-methyl-N-[3-(trifluoromethyl)-5,6-dihydro-4H-cyclopenta[b]thiophen-5-yl]carbamate (6) (300 mg, 933.54 μmol, 88.51% yield) was obtained as a colorless oil. ¹H NMR (400 MHz, CHLOROFORM-d) δ=7.55 (d, J=0.6 Hz, 1H), 5.05 (s, 1H), 3.25-3.05 (m, 2H), 2.99-2.69 (m, 5H), 1.47 (s, 9H). LC-MS: m/z [M-Boc+H]⁺=222.0

### Procedure for preparation of N-methyl-3-(trifluoromethyl)-5,6-dihydro-4H-cyclopenta[b]thiophen-5-amine hydrochloride (7)

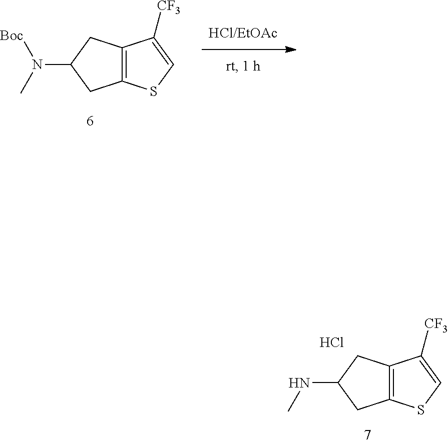 [SMILES: CN(C(=O)OC(C)(C)C)C1Cc2scc(C(F)(F)F)c2C1 ; CNC1Cc2scc(C(F)(F)F)c2C1 ; Cl]

To a solution of tert-butyl N-methyl-N-[3-(trifluoromethyl)-5,6-dihydro-4H-cyclopenta[b]thiophen-5-yl]carbamate (6) (300 mg, 933.54 μmol, 1 eq) in EtOAc (2 mL) was added HCl/EtOAc (2 M, 2 mL, 4.28 eq). The mixture was stirred at 25° C. for 1 hr. and then concentrated under vacuum to afford the product. Compound racemic N-methyl-3-(trifluoromethyl)-5,6-dihydro-4H-cyclopenta[b]thiophen-5-amine hydrochloride (7) (230 mg, 892.50 μmol, 95.60% yield) was obtained as a white solid. LC-MS: m/z [M+H]⁺=222.0

### Procedure for preparation of benzyl N-methyl-N-[3-(trifluoromethyl)-5,6-dihydro-4H-cyclopenta[b]thiophen-5-yl]carbamate (8)

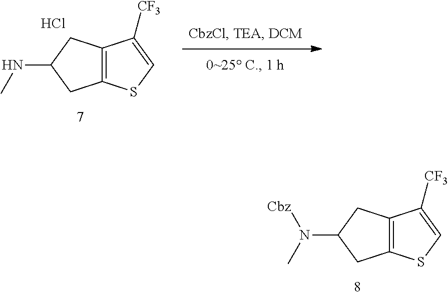 [SMILES: CNC1Cc2scc(C(F)(F)F)c2C1 ; Cl ; CN(C(=O)OCc1ccccc1)C1Cc2scc(C(F)(F)F)c2C1]

To a solution of N-methyl-3-(trifluoromethyl)-5,6-dihydro-4H-cyclopenta[b]thiophen-5-amine hydrochloride (7) (230 mg, 892.50 μmol, 1 eq) in DCM (5 mL) was added dropwise TEA (270.93 mg, 2.68 mmol, 372.67 μL, 3 eq) at 0° cand then dropwise CbzCl (182.71 mg, 1.07 mmol, 152.89 μL, 1.2 eq). The resulting mixture was stirred at 25° C. for 1 hr. Followed removal of the solvent under vacuum. The residue was purified by flash silica gel chromatography (ISCO®; 4 g SepaFlash® Silica Flash Column, eluent 0~10% Ethyl acetate/Petroleum ether gradient @40 mL/min). Compound benzyl N-methyl-N-[3-(trifluoromethyl)-5,6-dihydro-4H-cyclopenta[b]thiophen-5-yl]carbamate (8) (260 mg, 731.62 μmol, 81.97% yield) was obtained as a colorless oil. LC-MS: m/z [M+H]+=356.0

### Procedure for preparation of benzyl N-methyl-N-[3-(trifluoromethyl)-5,6-dihydro-4H-cyclopenta[b]thiophen-5-yl]carbamate enantiomer 1 (8a) and enantiomer2 (8b)

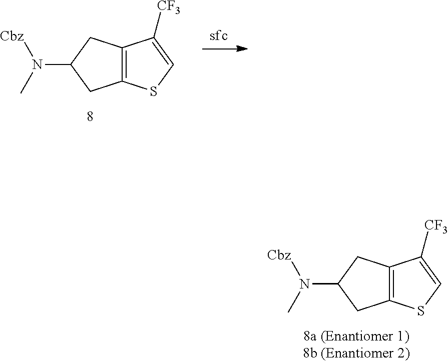 [SMILES: CN(C(=O)OCc1ccccc1)C1Cc2scc(C(F)(F)F)c2C1 ; "sfc" ; CN(C(=O)OCc1ccccc1)C1Cc2scc(C(F)(F)F)c2C1]

Racemic benzyl N-methyl-N-[3-(trifluoromethyl)-5,6-dihydro-4H-cyclopenta[b]thiophen-5-yl]carbamate (8) was separated to its enatiomers by SFC (column: DAICEL CHIRALPAK IG (250 mm*50 mm, 10 μm); mobile phase: [CO₂-EtOH (0.1% NH₃H₂O)]; B %:12%, isocratic elution mode) to afford enantiomer 1 (8a) (RT=1.001 min, 100 mg, 281.39 μmol, 38.46% yield) and enantiomer 2 (8b)(RT=1.057 min, 90 mg, 253.25 μmol, 34.62% yield) as colorless oils.

### Procedure for preparation of N-methyl-3-(trifluoromethyl)-5,6-dihydro-4H-cyclopenta[b]thiophen-5-amine hydrochloride enantiomer 1 (314)

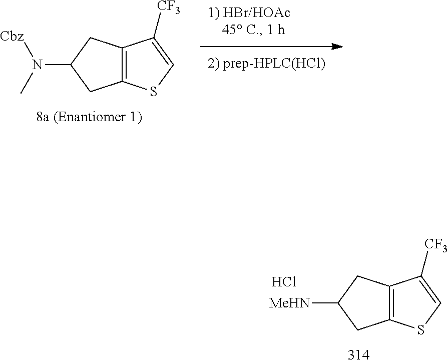 [SMILES: CN(C(=O)OCc1ccccc1)C1Cc2scc(C(F)(F)F)c2C1 ; Cl ; CNC1Cc2scc(C(F)(F)F)c2C1]

To a solution of benzyl N-methyl-N-[3-(trifluoromethyl)-5,6-dihydro-4H-cyclopenta[b]thiophen-5-yl]carbamate enantiomer 1 (8a) (100 mg, 281.39 μmol, 1 eq) in AcOH (1 mL) was added 33% HBr in acetic acid (1.49 g, 18.05 mmol, 1 mL, 64.13 eq). The mixture was stirred at 45° C. for 1 hr cooled and concentrated under reduced pressure to remove AcOH. Followed addition of MeOH and then TEA to neutrality. The mixture filtered and passed through prep-HPLC (column: Phenomenex luna C18 150*25 mm*10 μm; mobile phase: [water(HCl)-ACN]; gradient:1%-30% B over 10 min) to afford the product. Compound N-methyl-3-(trifluoromethyl)-5,6-dihydro-4H-cyclopenta[b]thiophen-5-amine hydrochloride enantiomer 1 (314) (40.62 mg, 157.62 μmol, 56.02% yield) was obtained as a white solid. ¹H NMR (400 MHz, METHANOL-d4) δ=7.92 (s, 1H), 4.48-4.38 (m, 1H), 3.49 (dd, J=7.9, 16.7 Hz, 1H), 3.38 (dd, J=8.0, 16.3 Hz, 1H), 3.13 (br dd, J=4.8, 16.6 Hz, 1H), 2.99 (dd, J=4.7, 16.2 Hz, 1H), 2.78 (s, 3H). LC-MS: m/z [M+H]⁺=222.0

### Procedure for preparation of N-methyl-3-(trifluoromethyl)-5,6-dihydro-4H-cyclopenta[b]thiophen-5-amine hydrochloride enantiomer 2 (315)

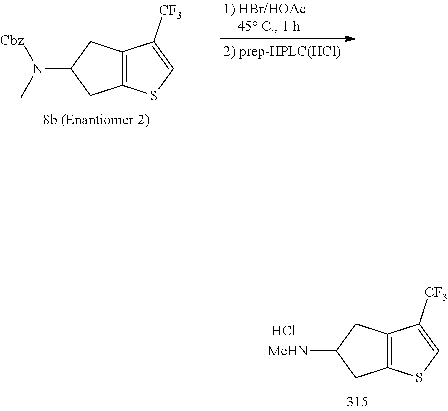 [SMILES: CN(C(=O)OCc1ccccc1)C1Cc2scc(C(F)(F)F)c2C1 ; Cl ; CNC1Cc2scc(C(F)(F)F)c2C1]

To a solution of benzyl N-methyl-N-[3-(trifluoromethyl)-5,6-dihydro-4H-cyclopenta[b]thiophen-5-yl]carbamate enantiomer 2 (8b) (90 mg, 253.25 μmol, 1 eq) in AcOH (1 mL) was added 33% HBr in acetic acid (1.49 g, 18.05 mmol, 1 mL, 71.26 eq). The mixture was stirred at 45° C. for 1 hr. The reaction mixture was concentrated under reduced pressure to remove AcOH. Followed addition of MeOH and TEA to neutrality. The mixture filtered and purified by prep-HPLC (column: Phenomenex luna C18 150*25 mm*10 μm; mobile phase: [water(HCl)-ACN]; gradient:1%-30% B over 10 min). Compound N-methyl-3-(trifluoromethyl)-5,6-dihydro-4H-cyclopenta[b]thiophen-5-amine hydrochloride enantiomer 2 (315) (43.05 mg, 167.05 μmol, 65.96% yield) was obtained as a white solid. ¹H NMR (400 MHz, METHANOL-d4) δ=7.92 (s, 1H), 4.48-4.38 (m, 1H), 3.49 (dd, J=8.0, 16.6 Hz, 1H), 3.38 (dd, J=8.0, 16.3 Hz, 1H), 3.12 (dd, J=4.8, 16.7 Hz, 1H), 2.99 (dd, J=4.8, 16.3 Hz, 1H), 2.78 (s, 3H). LC-MS: m/z [M+H]⁺=222.0

### Synthetic scheme for preparation of N, 3-dimethyl-5,6-dihydro-4H-cyclopenta[b]thiophen-5-amine hydrochloride enantiomer 1 (316) and enantiomer 2 (317)

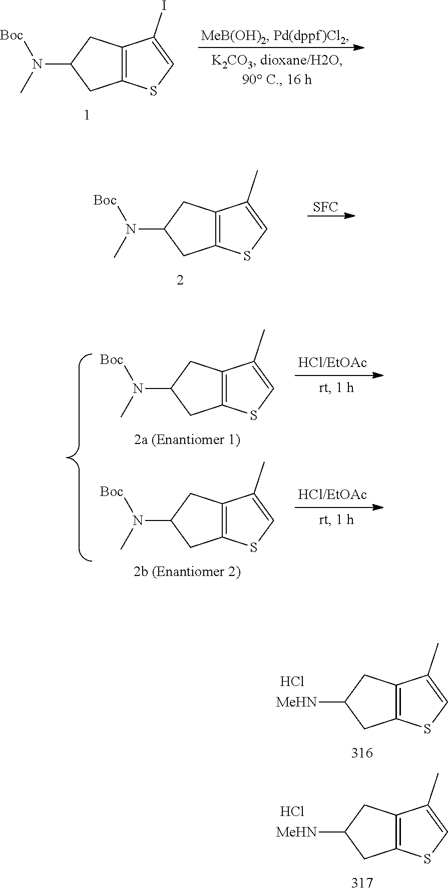

### Procedure for preparation of tert-butyl N-methyl-N-(3-methyl-5,6-dihydro-4H-cyclopenta[b]thiophen-5-yl)carbamate (2)

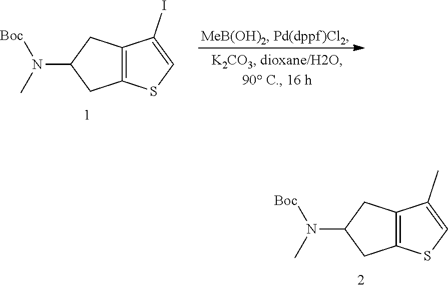

A mixture of tert-butyl N-(3-iodo-5,6-dihydro-4H-cyclopenta[b]thiophen-5-yl)-N-methyl-carbamate (1) (470 mg, 1.24 mmol, 1 eq), MeB(OH)₂ (741.83 mg, 12.39 mmol, 10 eq), Pd(dppf)Cl₂ (45.34 mg, 61.96 μmol, 0.05 eq) and K₂CO₃ (513.82 mg, 3.72 mmol, 3 eq) in dioxane (10 mL) and H₂O (1 mL) was degassed and purged with N₂. The mixture was then stirred at 90° C. for 16 h under N₂ atmosphere then cooled and diluted with H₂O (10 mL) and extracted with EtOAc (30 mL*2). The combined organic layers were washed with aq. sat.NaCl (10 mL), dried over anhydrous Na₂SO₄, filtered and concentrated under reduced pressure. The residue was purified by flash silica gel chromatography (ISCO®; 12 g SepaFlash® Silica Flash Column, eluent 0~10% Ethyl acetate/Petroleum ether gradient @40 mL/min). Compound tert-butyl N-methyl-N-(3-methyl-5,6-dihydro-4H-cyclopenta[b]thiophen-5-yl)carbamate (2) (150 mg, 560.99 μmol, 45.27% yield) was obtained as a colorless oil. ¹H NMR (400 MHz, CHLOROFORM-d) δ=6.76 (s, 1H), 5.66-5.20 (m, 1H), 3.25-3.07 (m, 1H), 2.96-2.84 (m, 2H), 2.78 (br s, 3H), 2.69-2.59 (m, 1H), 2.14 (s, 3H), 1.48 (s, 9H). LC-MS: m/z [M-tBu]⁺=212.0

### Procedure for preparation of tert-butyl N-methyl-N-(3-methyl-5,6-dihydro-4H-cyclopenta[b]thiophen-5-yl)carbamate enantiomer 1 (2a) and enantiomer 2 (2b)

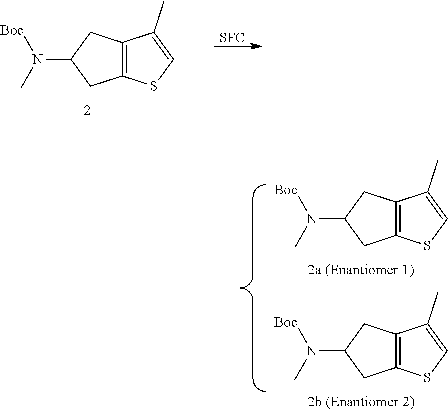

Racemic tert-butyl N-methyl-N-(3-methyl-5,6-dihydro-4H-cyclopenta[b]thiophen-5-yl)carbamate (2) was separated to its enantiomers by SFC column: DAICEL CHIRALPAK IG (250 mm*50 mm, 10 μm); mobile phase: [CO₂-EtOH (0.1% NH₃H₂O)]; B %:10%, isocratic elution mode). 1^{st} enantiomer 2a (RT=0.805 min, 50 mg, 187.00 μmol, 33.33% yield), and 2nd enantiomer 2b (RT=0.959 min, 60 mg, 224.39 μmol, 40.00% yield) were both isolated as colorless oils.

### Procedure for preparation of N, 3-dimethyl-5,6-dihydro-4H-cyclopenta[b]thiophen-5-amine hydrochloride enantiomer 1 (316)

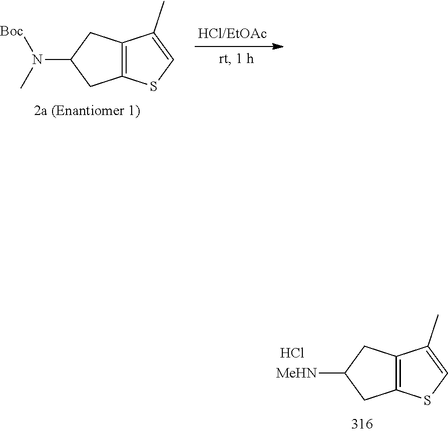 [SMILES: Cc1csc2c1CC(N(C)C(=O)OC(C)(C)C)C2 ; Cl ; CNC1Cc2scc(C)c2C1]

To a solution of tert-butyl N-methyl-N-(3-methyl-5,6-dihydro-4H-cyclopenta[b]thiophen-5-yl)carbamate enantiomer 1 (2a) (50 mg, 187.00 μmol, 1 eq) in EtOAc (2 mL) was added HCl/EtOAc (2 M, 2 mL, 21.39 eq). The mixture was stirred at 25° C. for 1 hr then filtered and the cake was washed with EtOAc (2 mL) dried and collected. Compound N, 3-dimethyl-5,6-dihydro-4H-cyclopenta[b]thiophen-5-amine enantiomer 1 (316) (24.74 mg, 119.21 μmol, 63.75% yield) was obtained as a white solid. ¹H NMR (400 MHz, METHANOL-d4) δ=6.92 (s, 1H), 4.39-4.29 (m, 1H), 3.41 (dd, J=7.8, 16.3 Hz, 1H), 3.19 (dd, J=7.7, 15.9 Hz, 1H), 3.04 (dd, J=5.0, 16.5 Hz, 1H), 2.85-2.72 (m, 4H), 2.16 (s, 3H). LC-MS: m/z [M+H]⁺=168.1

### Procedure for preparation of N, 3-dimethyl-5,6-dihydro-4H-cyclopenta[b]thiophen-5-amine hydrochloride enantiomer 2 (317)

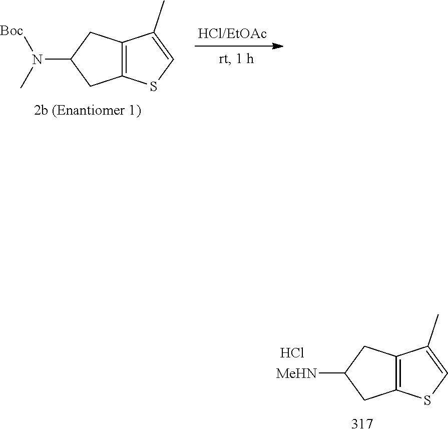 [SMILES: Cc1csc2c1CC(N(C)C(=O)OC(C)(C)C)C2 ; Cl ; CNC1Cc2scc(C)c2C1]

To a solution of tert-butyl N-methyl-N-(3-methyl-5,6-dihydro-4H-cyclopenta[b]thiophen-5-yl)carbamate enantiomer 2 (2b) (60 mg, 224.39 μmol, 1 eq) in EtOAc (2 mL) was added HCl/EtOAc (2 M, 2 mL, 17.83 eq). The mixture was stirred at 25° C. for 1 hr then filtered and the cake was washed with EtOAc (2 mL), filtered and collected. Compound N, 3-dimethyl-5,6-dihydro-4H-cyclopenta[b]thiophen-5-amine hydrochloride enantiomer 2 (317) (11.84 mg, 57.76 μmol, 25.74% yield) was obtained as a white solid. ¹H NMR (400 MHz, METHANOL-d4) δ=6.92 (s, 1H), 4.40-4.28 (m, 1H), 3.41 (dd, J=7.6, 16.5 Hz, 1H), 3.19 (dd, J=7.9, 15.9 Hz, 1H), 3.04 (br dd, J=5.0, 16.3 Hz, 1H), 2.84-2.74 (m, 4H), 2.16 (s, 3H). LC-MS: m/z [M+H]⁺=168.1

### Synthetic scheme for preparation of N, 2-dimethyl-5,6-dihydro-4H-cyclopenta[b]thiophen-5-amine hydrochloride enantiomer 1 (318) and enantiomer 2 (319)

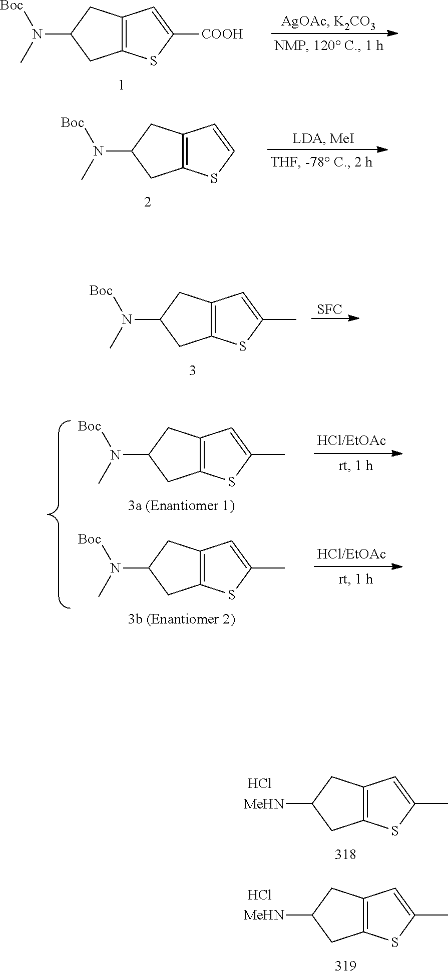

### Procedure for preparation of tert-butyl N-(5,6-dihydro-4H-cyclopenta[b]thiophen-5-yl)-N-methyl-carbamate (2)

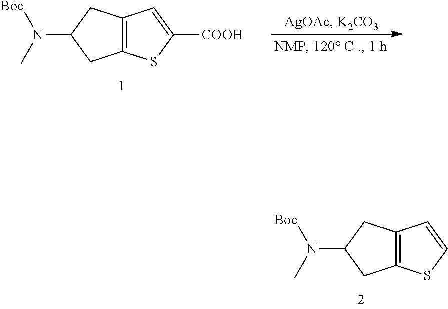

To a solution of 5-[tert-butoxycarbonyl(methyl)amino]-5,6-dihydro-4H-cyclopenta[b]thiophene-2-carboxylic acid (1) (740 mg, 2.49 mmol, 1 eq) in NMP (10 mL) was added AgOAc (41.54 mg, 248.85 μmol, 0.1 eq) and K₂CO₃ (1.03 g, 7.47 mmol, 3 eq). The mixture was stirred at 120° C. for 1 hr. The reaction mixture was then cooled, diluted with H₂O (50 mL) and extracted with EtOAc (30 mL*2). The combined organic layers were washed with aq. sat.NaCl (50 mL), dried over anhydrous Na₂SO₄, filtered and concentrated under reduced pressure. The residue was purified by flash silica gel chromatography (ISCO®; 12 g SepaFlash® Silica Flash Column, eluent 0~10% Ethyl acetate/Petroleum ether gradient @40 mL/min). Compound tert-butyl N-(5,6-dihydro-4H-cyclopenta[b]thiophen-5-yl)-N-methyl-carbamate (2) (470 mg, 1.86 mmol, 74.55% yield) was obtained as a colorless oil. ¹H NMR (400 MHz, CHLOROFORM-d) δ=7.18 (d, J=4.9 Hz, 1H), 6.81 (d, J=4.9 Hz, 1H), 5.71-5.11 (m, 1H), 3.17 (br dd, J=8.3, 15.7 Hz, 1H), 3.02 (br dd, J=8.7, 15.2 Hz, 1H), 2.92-2.71 (m, 5H), 1.48 (s, 9H). LC-MS: m/z [M-tBu+H]⁺=198.0

### Procedure for preparation of tert-butyl N-methyl-N-(2-methyl-5,6-dihydro-4H-cyclopenta[b]thiophen-5-yl)carbamate (3)

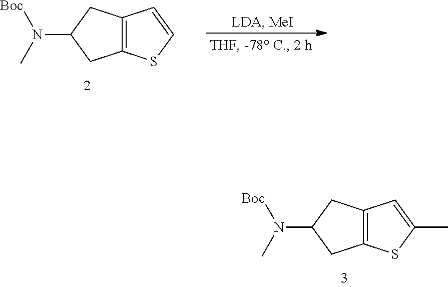

To a solution of tert-butyl N-(5,6-dihydro-4H-cyclopenta[b]thiophen-5-yl)-N-methyl-carbamate (2) (470 mg, 1.86 mmol, 1 eq) in THF (10 mL) at −78° C. was added dropwise LDA (2 M, 1.11 mL, 1.2 eq) The mixture was stirred at −78° C. for 1 hr. Then followed dropwise addition of CH₃I (315.97 mg, 2.23 mmol, 138.58 μL, 1.2 eq) at −78° C. The resulting mixture was stirred at −78° C. for 1 hr more and then quenched with aq. sat.NH₄Cl (20 mL) and extracted with EtOAc (30 mL*2). The combined organic layers were washed with aq. sat.NaCl (20 mL), dried over anhydrous Na₂SO₄, filtered and concentrated under reduced pressure to give a residue. The residue was purified by flash silica gel chromatography (ISCO®; 4 g SepaFlash® Silica Flash Column, eluent 0~10% Ethyl acetate/Petroleum ether gradient @40 mL/min). The residue was purified by prep-HPLC (column: Phenomenex luna C18 150*25 mm*10 μm; mobile phase: [water(FA)-ACN]; gradient:64%-94% B over 15 min). Compound tert-butyl N-methyl-N-(2-methyl-5,6-dihydro-4H-cyclopenta[b]thiophen-5-yl)carbamate (3) (110 mg, 411.39 μmol, 22.18% yield) was obtained as a colorless oil. LC-MS: m/z [M-tBu+H]⁺=212.0

### Procedure for preparation of tert-butyl N-methyl-N-(2-methyl-5,6-dihydro-4H-cyclopenta[b]thiophen-5-yl)carbamate enantiomer 1 (3a) and enantiomer 2 (3b)

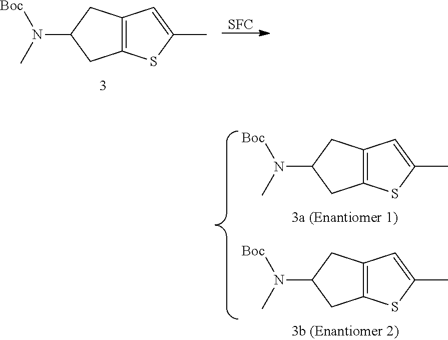

Racemic tert-butyl N-methyl-N-(2-methyl-5,6-dihydro-4H-cyclopenta[b]thiophen-5-yl)carbamate (3) was separated to its enantiomers by SFC column: DAICEL CHIRALPAK IG (column: DAICEL CHIRALPAK IC (250 mm*50 mm, 10 μm); mobile phase: [Hexane-IPA]; B %:10%, isocratic elution mode) to afford the 1^{st} enantiomer (3a) (RT=1.008 min, 35 mg, 130.90 μmol, 35.00% yield) and the 2nd enantiomer (3b) (RT=1.061 min, 30 mg, 112.20 μmol, 30.00% yield) as colorless oils.

### Procedure for preparation of N, 2-dimethyl-5,6-dihydro-4H-cyclopenta[b]thiophen-5-amine hydrochloride enantiomer 1 (318)

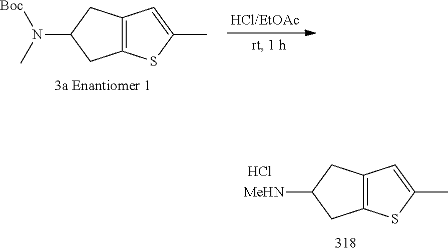

To a solution of tert-butyl N-methyl-N-(2-methyl-5,6-dihydro-4H-cyclopenta[b]thiophen-5-yl)carbamate enantiomer 1 (3a) (35 mg, 130.90 μmol, 1 eq) in EtOAc (2 mL) was added HCl/EtOAc (2 M, 2.33 mL, 35.65 eq). The mixture was stirred at 25° C. for 1 hr and then concentrated in vacuo to afford the product. Compound N, 2-dimethyl-5,6-dihydro-4H-cyclopenta[b]thiophen-5-amine hydrochloride enantiomer 1 (318) (23.08 mg, 112.15 μmol, 85.68% yield) was obtained as a white solid. ¹H NMR (400 MHz, METHANOL-d4) δ=6.56 (s, 1H), 4.34-4.23 (m, 1H), 3.41-3.33 (m, 1H), 3.20 (dd, J=7.8, 15.8 Hz, 1H), 3.05-2.95 (m, 1H), 2.83 (br dd, J=4.1, 15.8 Hz, 1H), 2.74 (s, 3H), 2.44 (s, 3H). LC-MS: m/z [M+H]⁺=168.1

### Procedure for preparation of N, 2-dimethyl-5,6-dihydro-4H-cyclopenta[b]thiophen-5-amine hydrochloride enantiomer 2 (319)

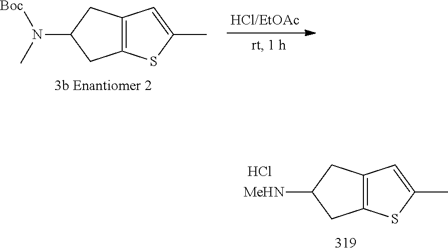

To a solution of tert-butyl N-methyl-N-(2-methyl-5,6-dihydro-4H-cyclopenta[b]thiophen-5-yl)carbamate enantiomer 2 (3b) (30 mg, 112.20 μmol, 1 eq) in EtOAc (2 mL) was added HCl/EtOAc (2 M, 1.71 mL, 30.56 eq). The mixture was stirred at 25° C. for 1 hr and then concentrated in vacuo to afford the product. Compound N, 2-dimethyl-5,6-dihydro-4H-cyclopenta[b]thiophen-5-amine hydrochloride enantiomer 2 (319) (16.66 mg, 81.42 μmol, 72.57% yield) was obtained as a white solid. ¹H NMR (400 MHz, METHANOL-d4) δ=6.56 (s, 1H), 4.36-4.23 (m, 1H), 3.44-3.33 (m, 1H), 3.20 (dd, J=7.8, 15.9 Hz, 1H), 3.04-2.94 (m, 1H), 2.84 (br dd, J=4.0, 15.5 Hz, 1H), 2.74 (s, 3H), 2.44 (s, 3H). LC-MS: m/z [M+H]⁺=168.1

### Synthetic scheme for preparation of 3-cyclopropyl-N-methyl-5,6-dihydro-4H-cyclopenta[b]thiophen-5-amine hydrochloride enantiomer 1 (320) and enantiomer 2 (321)

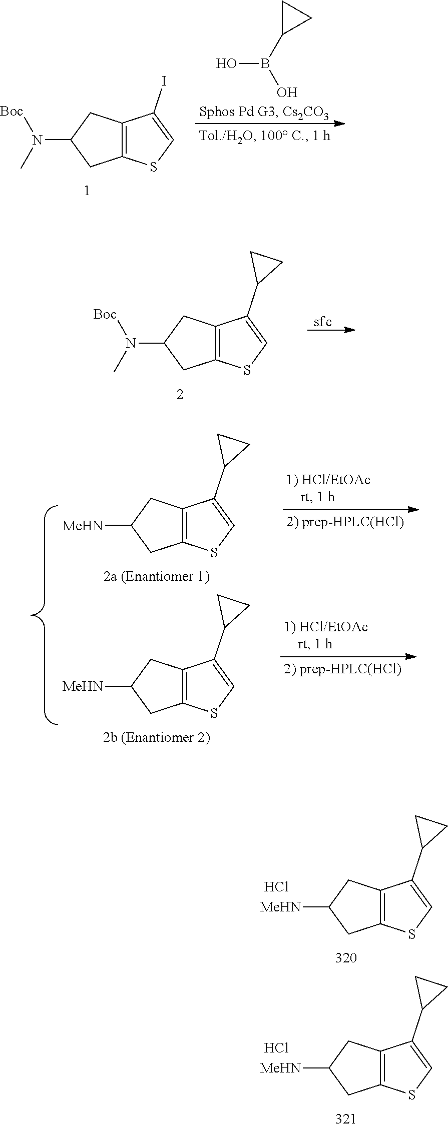

### Procedure for preparation of tert-butyl N-(3-cyclopropyl-5,6-dihydro-4H-cyclopenta[b]thiophen-5-yl)-N-methyl-carbamate (2)

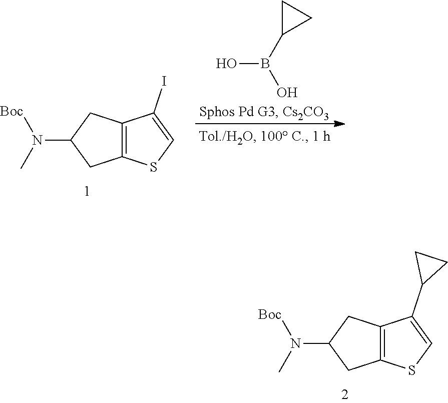

A mixture of tert-butyl N-(3-iodo-5,6-dihydro-4H-cyclopenta[b]thiophen-5-yl)-N-methyl-carbamate (1) (240 mg, 632.82 μmol, 1 eq), cyclopropylboronic acid (271.79 mg, 3.16 mmol, 5 eq), Sphos Pd G3 (49.38 mg, 63.28 μmol, 0.1 eq) and Cs₂CO₃ (618.55 mg, 1.90 mmol, 3 eq) in Tol. (2 mL) and H₂O (0.5 mL) was degassed and purged with N₂ for 3 times, and then the mixture was stirred at 100° C. for 2 hr. The reaction mixture was then cooled and diluted with H₂O (5 mL) and extracted with EtOAc (5 mL*2). The combined organic layers were washed with aq. sat.NaCl (5 mL), dried over anhydrous Na₂SO₄, filtered and concentrated under reduced pressure. The residue was purified by flash silica gel chromatography (ISCO®; 12 g SepaFlash® Silica Flash Column, eluent 0~9% Ethyl acetate/Petroleum ether gradient @40 mL/min) and then by prep-HPLC (column: Phenomenex luna C18 150*25 mm*10 μm; mobile phase: [water(FA)-ACN]; gradient:68%-98% B over 15 min). Compound tert-butyl N-(3-cyclopropyl-5,6-dihydro-4H-cyclopenta[b]thiophen-5-yl)-N-methyl-carbamate (2) (80 mg, 272.64 μmol, 43.08% yield) was obtained as a colorless oil. LC-MS: m/z [M+H]⁺=294.1

### Procedure for preparation of tert-butyl N-(3-cyclopropyl-5,6-dihydro-4H-cyclopenta[b]thiophen-5-yl)-N-methyl-carbamate enantiomer 1 (2a) and enantiomer 2 (2b)

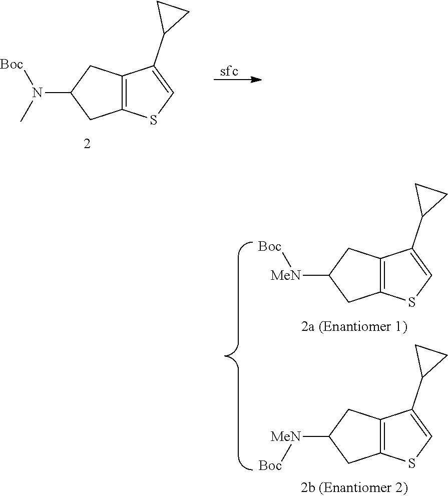

Racemic tert-butyl N-(3-cyclopropyl-5,6-dihydro-4H-cyclopenta[b]thiophen-5-yl)-N-methyl-carbamate (2) was separated to its enantiomers by SFC column: DAICEL CHIRALPAK IG (column: DAICEL CHIRALPAK IG (250 mm*30 mm, 10 μm); mobile phase: [Hexane-EtOH]; B %:10%, isocratic elution mode). The 1^{st} enantiomer 2a (RT=0.925 min, 35 mg, 119.28 μmol, 43.75% yield) and the 2nd enantiomer 2b (RT=1.005 min, 45 mg, 153.36 μmol, 56.25% yield) were obtained both as colorless oils.

### Procedure for preparation of 3-cyclopropyl-N-methyl-5,6-dihydro-4H-cyclopenta[b]thiophen-5-amine hydrochloride enantiomer 1 (320)

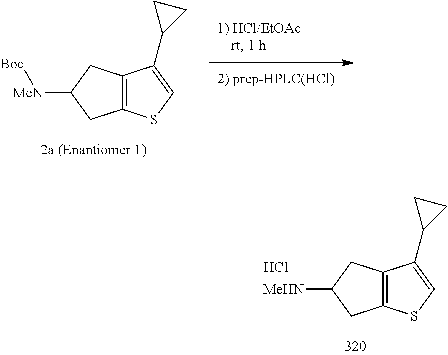 [SMILES: CN(C(=O)OC(C)(C)C)C1Cc2scc(C3CC3)c2C1 ; Cl ; CNC1Cc2scc(C3CC3)c2C1]

To a solution of tert-butyl N-(3-cyclopropyl-5,6-dihydro-4H-cyclopenta[b]thiophen-5-yl)-N-methyl-carbamate enantiomer 1 (2a) (35 mg, 119.28 μmol, 1 eq) in EtOAc (2 mL) was added HCl/EtOAc (2 M, 2 mL, 33.53 eq). The mixture was stirred at 25° C. for 1 hr and then the solvent was removed under vacuum. The residue was purified by prep-HPLC (column: Phenomenex luna C18 150*25 mm*10 μm; mobile phase: [water(HCl)-ACN]; gradient:3%-33% B over 10 min). Compound 3-cyclopropyl-N-methyl-5,6-dihydro-4H-cyclopenta[b]thiophen-5-amine hydrochloride enantiomer 1 (320) (10.65 mg, 46.19 μmol, 38.73% yield) was obtained as a white solid. ¹H NMR (400 MHz, METHANOL-d4) δ=6.82 (s, 1H), 4.35 (br dd, J=3.9, 7.9 Hz, 1H), 3.39 (br dd, J=7.7, 16.3 Hz, 1H), 3.29-3.22 (m, 1H), 3.11-2.97 (m, 1H), 2.91-2.83 (m, 1H), 2.77 (s, 3H), 1.85-1.71 (m, 1H), 0.92-0.82 (m, 2H), 0.69-0.57 (m, 2H). LC-MS: m/z [M+H]⁺=194.0

### Procedure for preparation of 3-cyclopropyl-N-methyl-5,6-dihydro-4H-cyclopenta[b]thiophen-5-amine hydrochloride enantiomer 2 (321)

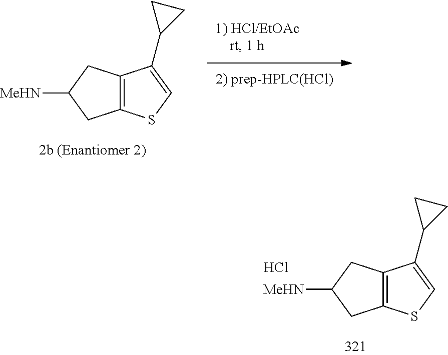 [SMILES: CNC1Cc2scc(C3CC3)c2C1 ; Cl ; CNC1Cc2scc(C3CC3)c2C1]

To a solution of tert-butyl N-(3-cyclopropyl-5,6-dihydro-4H-cyclopenta[b]thiophen-5-yl)-N-methyl-carbamate enantiomer 2 (2b) (45 mg, 153.36 μmol, 1 eq) in EtOAc (2 mL) was added HCl/EtOAc (2 M, 2 mL, 26.08 eq). The mixture was stirred at 25° C. for 1 hr and then the solvent was removed under vacuum. The residue was purified by prep-HPLC (column: Phenomenex luna C18 150*25 mm*10 μm; mobile phase: [water(HCl)-ACN]; gradient:3%-33% B over 10 min). Compound 3-cyclopropyl-N-methyl-5,6-dihydro-4H-cyclopenta[b]thiophen-5-amine hydrochloride enantiomer 2 (321) (8.16 mg, 35.34 μmol, 23.04% yield, 99.51%) was obtained as a white solid. ¹H NMR (400 MHz, METHANOL-d4) δ=6.82 (s, 1H), 4.39-4.29 (m, 1H), 3.39 (br dd, J=7.9, 16.3 Hz, 1H), 3.29-3.22 (m, 1H), 3.08-2.98 (m, 1H), 2.86 (br dd, J=4.1, 15.9 Hz, 1H), 2.76 (s, 3H), 1.86-1.70 (m, 1H), 0.91-0.80 (m, 2H), 0.68-0.52 (m, 2H). LC-MS: m/z [M+H]⁺=194.0

### Synthetic scheme for preparation of 3-(methylamino)-3,4-dihydro-2H-thieno[2,3-b]pyran-5-carbonitrile hydrochloride (322)

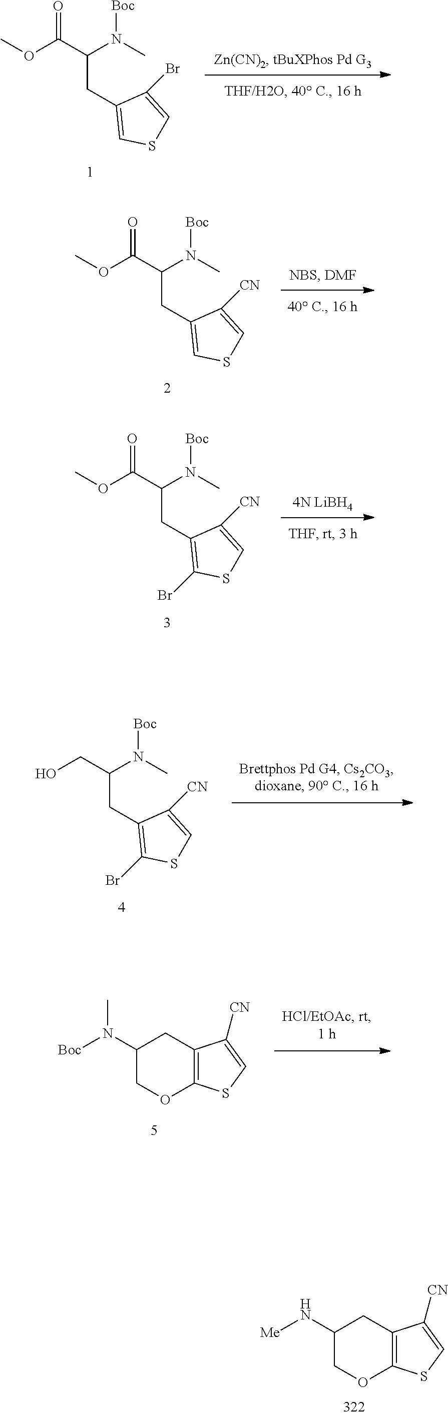

### Procedure for preparation of methyl 2-((tert-butoxycarbonyl)(methyl)amino)-3-(4-cyanothiophen-3-yl)propanoate (2)

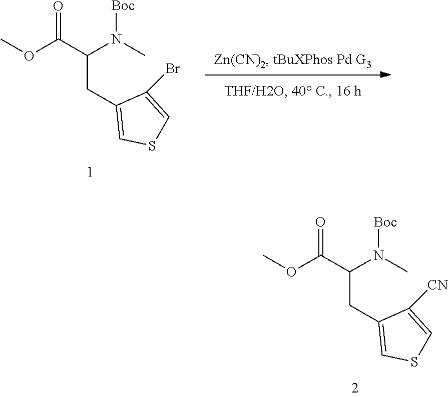 [SMILES: COC(=O)C(Cc1cscc1Br)N(C)C(=O)OC(C)(C)C ; "Zn(CN)2, tBuXPhos Pd G3" ; COC(=O)C(Cc1cscc1C#N)N(C)C(=O)OC(C)(C)C]

To a solution of methyl 3-(4-bromo-3-thienyl)-2-[tert-butoxycarbonyl(methyl)amino]propanoate (1) (4 g, 10.57 mmol, 1 eq) in THF (20 mL), H₂O (20 mL) was added tBuXPhos Pd G3 (839.98 mg, 1.06 mmol, 0.1 eq) and Zn(CN)₂ (1.75 g, 14.90 mmol, 945.95 μL, 1.41 eq). The mixture was stirred at 40° C. for 16 hr and then quenched by addition of aq. sat. NaHCO₃ (30 mL) at 25° C. The resulting mixture was extracted with EtOAc (30 mL*2) and the combined organic layers were washed with aq. sat. NaCl (30 mL*2), dried over Na₂SO₄, filtered and concentrated under reduced pressure. The residue was purified by flash silica gel chromatography (ISCO®; 20 g SepaFlash® Silica Flash Column, eluent 0~13% Ethyl acetate/Petroleum ether gradient @60 mL/min). Compound methyl 2-[tert-butoxycarbonyl(methyl)amino]-3-(4-cyano-3-thienyl)propanoate (2) (2 g, 6.04 mmol, 57.14% yield) was obtained as a light yellow oil. ¹H NMR (400 MHz, CHLOROFORM-d) δ=7.91 (br d, J=10.1 Hz, 1H), 7.23-7.10 (m, 1H), 5.00 (br dd, J=4.9, 10.4 Hz, 0.5H), 4.65 (br dd, J=4.4, 10.6 Hz, 0.5H), 3.77 (s, 3H), 3.42 (dd, J=5.0, 15.2 Hz, 1H), 3.28-3.13 (m, 1H), 2.77 (br d, J=9.3 Hz, 3H), 1.40 (br d, J=11.2 Hz, 9H).

### Procedure for preparation of methyl 3-(2-bromo-4-cyanothiophen-3-yl)-2-((tert-butoxycarbonyl)(methyl)amino)propanoate (3)

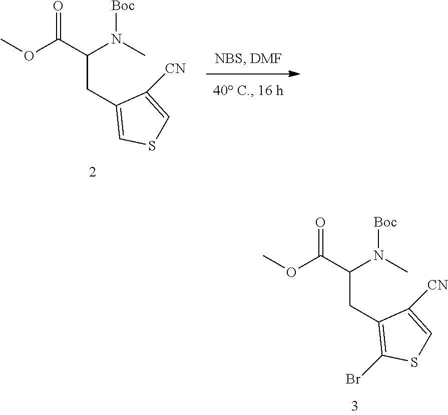

To a solution of methyl 2-[tert-butoxycarbonyl(methyl)amino]-3-(4-cyano-3-thienyl)propanoate (2) (2 g, 6.17 mmol, 1 eq) in DMF (20 mL) was added NBS (1.65 g, 9.25 mmol, 1.5 eq). The mixture was stirred at 40° C. for 16 hr then cooled and poured into H₂O (80 mL). The resulting mixture was extracted with EtOAc (30 mL*3) and the combined organic layers were washed with aq. saturated NaCl (50 mL*2), dried over anhydrous Na₂SO₄, filtered and concentrated under reduced pressure. The residue was purified by flash silica gel chromatography (ISCO®; 20 g SepaFlash® Silica Flash Column, eluent 0~9% Ethyl acetate/Petroleum ether gradient @60 mL/min). Compound methyl 3-(2-bromo-4-cyano-3-thienyl)-2-[tert-butoxycarbonyl(methyl)amino]propanoate (3) (1.7 g, 3.88 mmol, 62.90% yield) was obtained as a light yellow oil. ¹H NMR (400 MHz, CHLOROFORM-d) δ=7.89-7.82 (m, 1H), 4.95-4.60 (m, 1H), 3.79 (s, 3H), 3.39-3.18 (m, 2H), 2.83 (s, 3H), 1.39 (s, 9H).

### Procedure for preparation of tert-butyl (1-(2-bromo-4-cyanothiophen-3-yl)-3-hydroxypropan-2-yl)(methyl)carbamate (4)

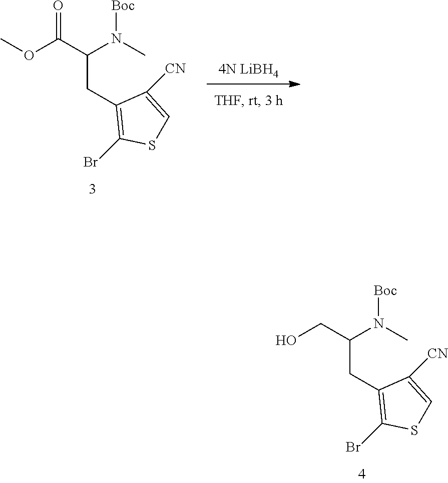

To a solution of methyl 3-(2-bromo-4-cyano-3-thienyl)-2-[tert-butoxycarbonyl(methyl)amino]propanoate (3) (1.7 g, 4.22 mmol, 1 eq) in THF (15 mL) was added LiBH₄ (4 M in THF, 1.6 mL, 1.52 eq) at 0° C. The mixture was then stirred at 25° C. for 3 hr and then quenched with aq. sat NH₄Cl (40 mL) at 0° C. The mixture was then extracted with EtOAc (30 mL*2). The combined organic layers were washed with aq. saturated NaCl (30 mL*2), dried over anhydrous Na₂SO₄, filtered and concentrated under reduced pressure. The residue was purified by flash silica gel chromatography (ISCO®; 20 g SepaFlash® Silica Flash Column, eluent 0~30% Ethyl acetate/Petroleum ether gradient @60 mL/min). Compound tert-butyl N-[1-[(2-bromo-4-cyano-3-thienyl)methyl]-2-hydroxy-ethyl]-N-methyl-carbamate (4) (0.6 g, 1.57 mmol, 37.17% yield) was obtained as a colorless oil. ¹H NMR (400 MHz, CHLOROFORM-d) δ=7.86 (br s, 1H), 4.54-4.38 (m, 0.5H), 4.26 (br d, J=2.9 Hz, 0.5H), 3.84-3.65 (m, 2H), 3.04-2.77 (m, 5H), 1.44-1.30 (m, 9H)

### Procedure for preparation of tert-butyl (5-cyano-3,4-dihydro-2H-thieno[2,3-b]pyran-3-yl)(methyl)carbamate (5)

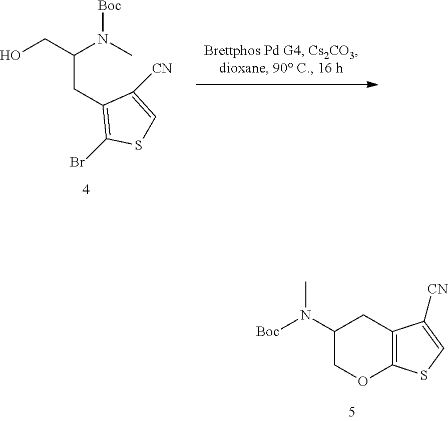 [SMILES: CN(C(=O)OC(C)(C)C)C(CO)Cc1c(C#N)csc1Br ; CN(C(=O)OC(C)(C)C)C1COc2scc(C#N)c2C1]

A mixture of tert-butyl N-[1-[(2-bromo-4-cyano-3-thienyl)methyl]-2-hydroxy-ethyl]-N-methyl-carbamate (4) (0.6 g, 1.60 mmol, 1 eq), BrettPhos Pd, G4 (147.17 mg, 159.88 μmol, 0.1 eq), Cs₂CO₃ (1.56 g, 4.80 mmol, 3 eq) in dioxane (40 mL) was degassed and purged with N₂ and stirred at 90° C. under N₂ atmosphere for 16 hr. The reaction mixture was then cooled, filtered and concentrated under reduced pressure. The residue was purified by flash silica gel chromatography (ISCO®; 12 g SepaFlash® Silica Flash Column, eluent 0~6% Ethyl acetate/Petroleum ether gradient @40 mL/min). Compound tert-butyl N-(5-cyano-3,4-dihydro-2H-thieno[2,3-b]pyran-3-yl)-N-methyl-carbamate (5) (280 mg, 913.14 μmol, 57.11% yield) was obtained as a light yellow oil. ¹H NMR (400 MHz, CHLOROFORM-d) δ=7.16 (s, 1H), 4.70-4.37 (m, 1H), 4.30-4.14 (m, 2H), 2.96-2.87 (m, 2H), 2.83 (s, 3H), 1.49 (s, 9H).

### Procedure for preparation of 3-(methylamino)-3,4-dihydro-2H-thieno[2,3-b]pyran-5-carbonitrile (322)

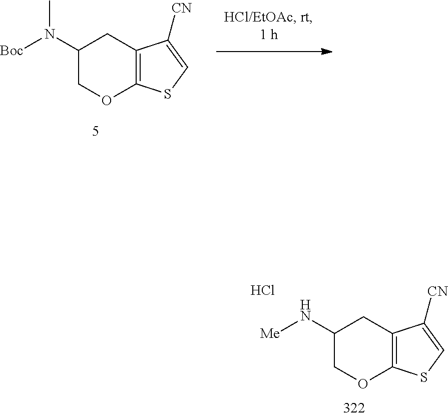 [SMILES: CN(C(=O)OC(C)(C)C)C1COc2scc(C#N)c2C1 ; Cl ; CNC1COc2scc(C#N)c2C1]

To a solution of tert-butyl N-(5-cyano-3,4-dihydro-2H-thieno[2,3-b]pyran-3-yl)-N-methyl-carbamate (5) (30 mg, 101.91 μmol, 1 eq) in EtOAc (0.5 mL) was added HCl/EtOAc (2 M, 2 mL, 39.25 eq). The mixture was stirred at 25° C. for 1 hr and then concentrated. The residue was triturated with EtOAc (1 mL) at 25° C. for 3 min then collected and dried to afford the product. Compound 3-(methylamino)-3,4-dihydro-2H-thieno[2,3-b]pyran-5-carbonitrile hydrochloride (322) (19.21 mg, 82.85 μmol, 81.29% yield) was obtained as a white solid. ¹H NMR (400 MHz, METHANOL-d₄) δ=7.62 (s, 1H), 4.70-4.64 (m, 1H), 4.37 (d, J=12.5 Hz, 1H), 3.89 (br d, J=2.3 Hz, 1H), 3.26 (dd, J=5.8, 17.4 Hz, 1H), 2.95 (td, J=2.0, 17.5 Hz, 1H), 2.85 (s, 3H). LC-MS: m/z [M+H]⁺=195.0

### Synthetic scheme for preparation of 5,6,7,8-tetrahydro-4H-cyclohepta[b]thiophen-7-amine hydrochloride (323)

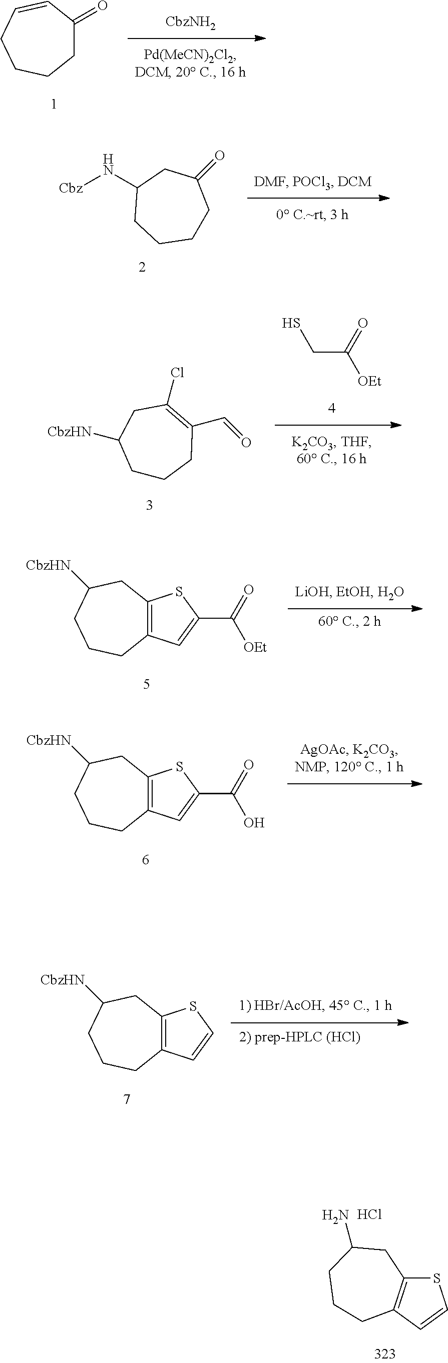

### Procedure for preparation of benzyl N-(3-oxocycloheptyl)carbamate (2)

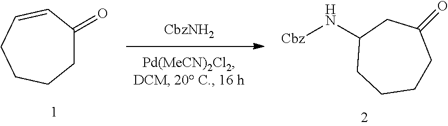 [SMILES: "CbzNH2" ; O=C1C=CCCCC1 ; O=C1CCCCC(NC(=O)OCc2ccccc2)C1]

To a solution of acetonitrile;dichloropalladium complex (693.38 mg, 2.67 mmol) in DCM (50 mL) was added cyclohept-2-en-1-one (3 g, 27.23 mmol, 3.00 mL, 1 eq) and CbzNH₂ (5.97 g, 39.52 mmol, 1.45 eq). The mixture was stirred at 20° C. for 16 hr. The reaction mixture was concentrated under reduced pressure to remove DCM. The residue was purified by flash silica gel chromatography (ISCO®; 40 g SepaFlash® Silica Flash Column, eluent 0~25% Ethyl acetate/Petroleum ether gradient @60 mL/min). Compound benzyl N-(3-oxocycloheptyl)carbamate (2) (5 g, 19.13 mmol, 70.26% yield) was obtained as a white solid. ¹H NMR (400 MHz, CHLOROFORM-d) δ=7.38-7.34 (m, 5H), 5.11 (br s, 2H), 4.02 (br d, J=2.4 Hz, 1H), 2.88 (br d, J=13.6 Hz, 1H), 2.68-2.43 (m, 3H), 2.04-1.94 (m, 1H), 1.91-1.70 (m, 5H). LC-MS: m/z [M+H]⁺=262.1

### Procedure for preparation of benzyl N-(3-chloro-4-formyl-cyclohept-3-en-1-yl)carbamate (3)

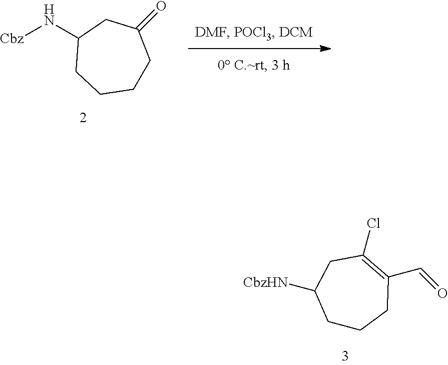 [SMILES: O=C1CCCCC(NC(=O)OCc2ccccc2)C1 ; O=CC1=C(Cl)CC(NC(=O)OCc2ccccc2)CCC1]

To a solution of DMF (2.80 g, 38.27 mmol, 2.94 mL, 5 eq) in DCM (10 mL) was added POCl₃ (7.04 g, 45.92 mmol, 4.28 mL, 6 eq) dropwise at 0° C. The mixture then was stirred at 25° C. for 1 h and then a solution of benzyl N-(3-oxocycloheptyl)carbamate (2) (2 g, 7.65 mmol, 1 eq) in DCM (10 mL) was added dropwise at 0° C. The resulting mixture was stirred at 25° C. for 2 h and then quenched by addition of aq. saturated NaHCO₃ (200 mL) at 25° C. and extracted with EtOAc (15 mL*2). The combined organic layers were washed with aq. sat. NaCl (10 mL*2), dried over Na₂SO₄, filtered and concentrated under reduced pressure. The residue was purified by flash silica gel chromatography (ISCO®; 40 g SepaFlash® Silica Flash Column, eluent 0~20% Ethyl acetate/Petroleum ether gradient @60 mL/min). Compound benzyl N-(3-chloro-4-formyl-cyclohept-3-en-1-yl)carbamate (3) (1.3 g, 4.22 mmol, 55.19% yield) was obtained as a yellow liquid. ¹H NMR (400 MHz, CHLOROFORM-d) δ=10.04 (s, 1H), 7.33-7.27 (m, 5H), 5.08-4.99 (m, 2H), 3.89-3.76 (m, 1H), 3.06-2.93 (m, 2H), 2.52-2.30 (m, 2H), 1.70 (br s, 2H), 1.56-1.35 (m, 2H).

### Procedure for preparation of ethyl 7-(benzyloxycarbonylamino)-5,6,7,8-tetrahydro-4H-cyclohepta[b]thiophene-2-carboxylate (5)

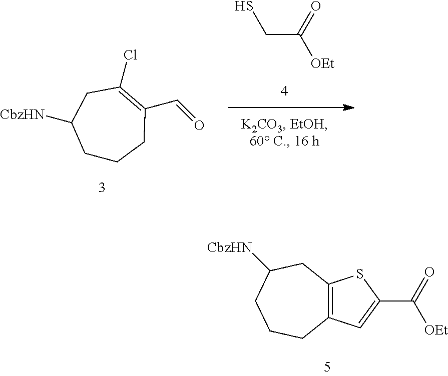 [SMILES: O=CC1=C(Cl)CC(NC(=O)OCc2ccccc2)CCC1 ; CCOC(=O)CS ; CCOC(=O)c1cc2c(s1)CC(NC(=O)OCc1ccccc1)CCC2]

To a solution of benzyl N-(3-chloro-4-formyl-cyclohept-3-en-1-yl)carbamate (3) (1.3 g, 4.22 mmol, 1 eq) in EtOH (20 mL) was added K₂CO₃ (875.65 mg, 6.34 mmol, 1.5 eq) and ethyl 2-sulfanylacetate (609.10 mg, 5.07 mmol, 555.75 μL, 1.2 eq). The mixture was stirred at 60° C. for 16 hr and then cooled and concentrated under reduced pressure to remove EtOH. The residue was purified by flash silica gel chromatography (ISCO®; 40 g SepaFlash® Silica Flash Column, eluent 0~20% Ethyl acetate/Petroleum ether gradient @60 mL/min). Compound ethyl 7-(benzyloxycarbonylamino)-5,6,7,8-tetrahydro-4H-cyclohepta[b]thiophene-2-carboxylate (5) (900 mg, 2.41 mmol, 57.05% yield) was obtained as a white solid. LC-MS: m/z [M+H]⁺=374.1

### Procedure for preparation of 7-(benzyloxycarbonylamino)-5,6,7,8-tetrahydro-4H-cyclohepta[b]thiophene-2-carboxylic acid (6)

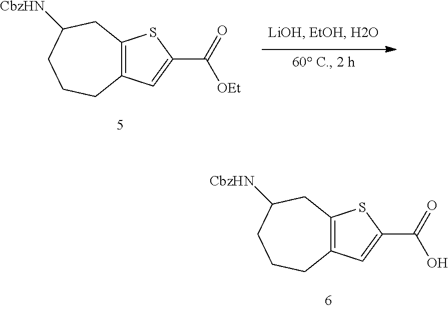 [SMILES: CCOC(=O)c1cc2c(s1)CC(NC(=O)OCc1ccccc1)CCC2 ; O=C(NC1CCCc2cc(C(=O)O)sc2C1)OCc1ccccc1]

To a solution of ethyl 7-(benzyloxycarbonylamino)-5,6,7,8-tetrahydro-4H-cyclohepta[b]thiophene-2-carboxylate (5) (900 mg, 2.41 mmol, 1 eq) in H₂O (10 mL) and EtOH (10 mL) was added LiOH·H₂O (404.51 mg, 9.64 mmol, 4 eq). The mixture was stirred at 60° C. for 2 hr then cooled and concentrated under reduced pressure to remove EtOH and the resulting mixture pH was adjusted to 6 with aq. 1N HCl. The solid formed was filtered collected and dried. Compound 7-(benzyloxycarbonylamino)-5,6,7,8-tetrahydro-4H-cyclohepta[b]thiophene-2-carboxylic acid (6) (600 mg, 1.74 mmol, 72.08% yield) was obtained as a white solid.

### Procedure for preparation of benzyl N-(5,6,7,8-tetrahydro-4H-cyclohepta[b]thiophen-7-yl)carbamate (7)

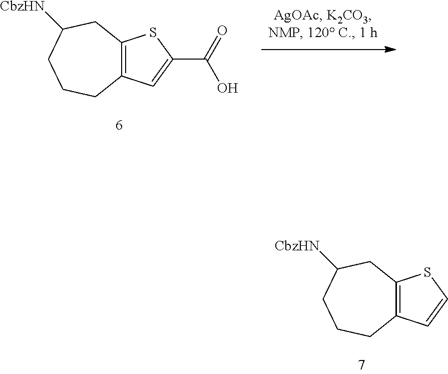 [SMILES: O=C(NC1CCCc2cc(C(=O)O)sc2C1)OCc1ccccc1 ; O=C(NC1CCCc2ccsc2C1)OCc1ccccc1]

To a solution of 7-(benzyloxycarbonylamino)-5,6,7,8-tetrahydro-4H-cyclohepta[b]thiophene-2-carboxylic acid (6) (600 mg, 1.74 mmol, 1 eq) in NMP (10 mL) was added AgOAc (144.97 mg, 868.53 μmol, 0.5 eq) and K₂CO₃ (720.21 mg, 5.21 mmol, 3 eq). The mixture was stirred at 120° C. for 1 hr then cooled and filtered. To the filtrate was added H₂O (200 ml) and the mixture was extracted with EtOAc (50 mL*2). The combined organic layers were washed with aq. sat. NaCl (50 mL*2), dried over Na₂SO₄, filtered and concentrated under reduced pressure. The residue was purified by flash silica gel chromatography (ISCO®; 20 g SepaFlash® Silica Flash Column, eluent 0~20% Ethyl acetate/Petroleum ether gradient @40 mL/min). Compound benzyl N-(5,6,7,8-tetrahydro-4H-cyclohepta[b]thiophen-7-yl)carbamate (7) (300 mg, 995.35 μmol, 57.30% yield) was obtained as a white solid. LC-MS: m/z [M+H]⁺=302.1

### Procedure for preparation of 5,6,7,8-tetrahydro-4H-cyclohepta[b]thiophen-7-amine hydrochloride (323)

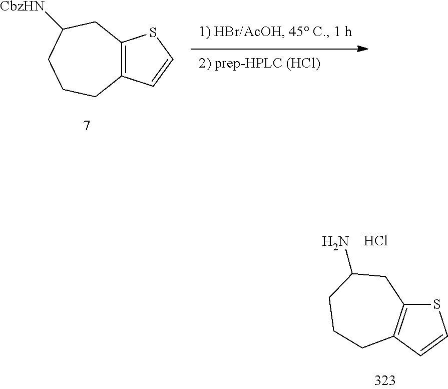 [SMILES: O=C(NC1CCCc2ccsc2C1)OCc1ccccc1 ; Cl ; NC1CCCc2ccsc2C1]

To a solution of benzyl N-(5,6,7,8-tetrahydro-4H-cyclohepta[b]thiophen-7-yl)carbamate (7) (50 mg, 165.89 μmol, 1 eq) in AcOH (1 mL) was added HBr in acetic acid (1.49 g, 6.08 mmol, 1 mL, 33%, 36.63 eq). The mixture was stirred at 45° C. for 1 hr then cooled and concentrated The residue was dissolved in MeOH. The mixture was treated with TEA to neutralize the acid and then filtered Followed prep-HPLC (HCl condition:column: Phenomenex luna C18 150*25 mm*10 μm; mobile phase: [water(HCl)-ACN]; gradient:1%-25% B over 10 min).Compound 5,6,7,8-tetrahydro-4H-cyclohepta[b]thiophen-7-amine hydrochloride (323) (14.63 mg, 71.04 μmol, 42.82% yield) was obtained as a white solid. ¹H NMR (400 MHz, METHANOL-d₄) δ=7.08 (d, J=5.1 Hz, 1H), 6.89-6.80 (m, 1H), 3.32-3.28 (m, 1H), 3.28-3.05 (m, 2H), 2.96-2.84 (m, 1H), 2.68 (ddd, J=1.9, 11.0, 15.1 Hz, 1H), 2.36 (s, 1H), 2.04-1.88 (m, 2H), 1.68-1.53 (m, 1H). LC-MS: m/z [M+H]⁺=168.1

### Synthetic scheme for preparation of 3-cyclopropyl-N-methyl-6,7-dihydro-5H-thieno[3,2-b]pyran-6-amine hydrochloride enantiomer 1 (324) and enantiomer 2 (325)

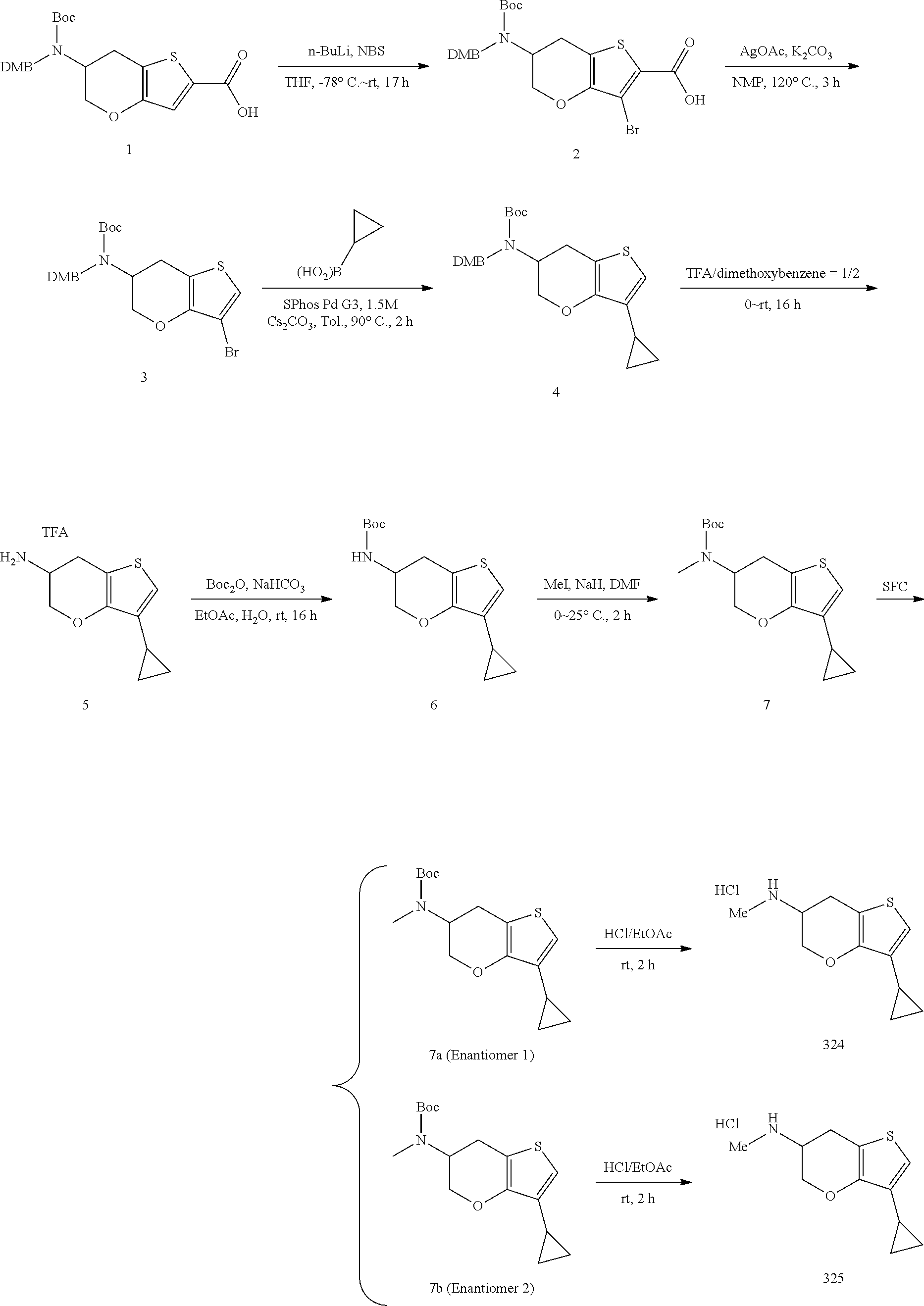

### Procedure for preparation of 3-bromo-6-[tert-butoxycarbonyl-[(2,4-dimethoxyphenyl)methyl]amino]-6,7-dihydro-5H-thieno[3,2-b]pyran-2-carboxylic acid (2)

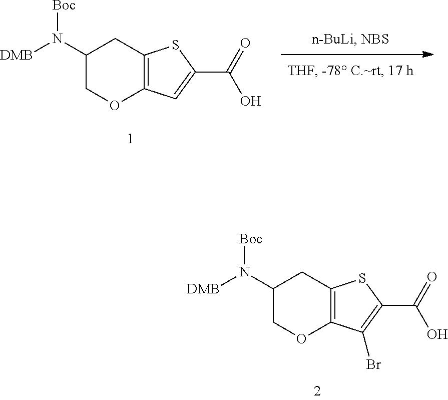

To a solution of 6-[tert-butoxycarbonyl-[(2,4-dimethoxyphenyl)methyl]amino]-6,7-dihydro-5H-thieno[3,2-b]pyran-2-carboxylic acid (1) (2.1 g, 4.67 mmol, 1 eq) in THF (20 mL) was added dropwise n-BuLi (2.5 M, 4.30 mL, 2.3 eq) at −78° C. over 0.5 hr. The mixture was stirred at this temperature for 0.5 hr, and then NBS (1.25 g, 7.01 mmol, 1.5 eq) in THF (5 mL) was added dropwise at −78° C. The resulting mixture was allowed to gradually warm up to rt, stirred for 16 hr and then poured into NH₄Cl aq. saturated solution (50 mL). This mixture was then extracted with EtOAc (50 mL*2). The combined organic phases were washed with brine (30 mL), dried over anhydrous sodium sulfate, filtered and concentrated under vacuum to give a residue. The residue was purified by prep-HPLC (column: Phenomenex luna C18 150*40 mm*15 μm; mobile phase: [water(FA)-ACN]; gradient:52%-82% B over 15 min) to afford 3-bromo-6-[tert-butoxycarbonyl-[(2,4-dimethoxyphenyl)methyl]amino]-6,7-dihydro-5H-thieno[3,2-b]pyran-2-carboxylic acid (2) (680 mg, 1.29 mmol, 27.55% yield) as yellow oil. ¹H NMR (400 MHz, CHLOROFORM-d) δ=7.21-7.06 (m, 1H), 6.52-6.37 (m, 2H), 4.54-4.04 (m, 5H), 3.80 (d, J=13.4 Hz, 6H), 3.38-3.10 (m, 1H), 2.88-2.71 (m, 1H), 1.49 (br s, 9H)

### Procedure for preparation of tert-butyl N-(3-bromo-6,7-dihydro-5H-thieno[3,2-b]pyran-6-yl)-N-[(2,4-dimethoxyphenyl)methyl]carbamate (3)

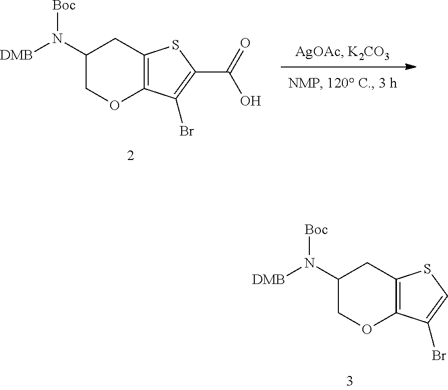

A mixture of 3-bromo-6-[tert-butoxycarbonyl-[(2,4-dimethoxyphenyl)methyl]amino]-6,7-dihydro-5H-thieno[3,2-b]pyran-2-carboxylic acid (2) (680 mg, 1.29 mmol, 1 eq), K₂CO₃ (533.56 mg, 3.86 mmol, 3 eq), AgOAc (42.96 mg, 257.37 μmol, 0.2 eq) in 1-methylpyrrolidin-2-one (8 mL) was degassed and purged with N₂ for 3 times and stirred at 120° C. for 3 hr. The reaction mixture was then cooled and poured into H₂O (30 mL) and extracted with EtOAc (30 mL*2). The combined organic phases were washed with brine (10 mL), dried over anhydrous sodium sulfate, filtered and concentrated under vacuum to give a residue. The residue was purified by flash silica gel chromatography (ISCO®; 12 g SepaFlash® Silica Flash Column, eluent 0~20% Ethyl acetate/Petroleum ether gradient @40 mL/min) to afford tert-butyl N-(3-bromo-6,7-dihydro-5H-thieno[3,2-b]pyran-6-yl)-N-[(2,4-dimethoxyphenyl)methyl]carbamate (3) (540 mg, 1.11 mmol, 86.63% yield) as yellow oil. LC-MS: m/z [M+Na]⁺=508.0

### Procedure for preparation of tert-butyl N-(3-cyclopropyl-6,7-dihydro-5H-thieno[3,2-b]pyran-6-yl)-N-[(2,4-dimethoxyphenyl)methyl]carbamate (4)

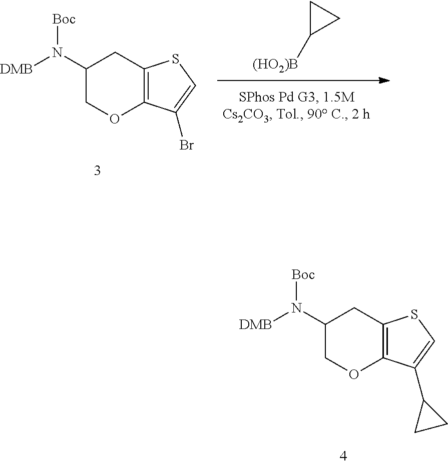

A mixture of tert-butyl N-(3-bromo-6,7-dihydro-5H-thieno[3,2-b]pyran-6-yl)-N-[(2,4-dimethoxyphenyl)methyl]carbamate (3) (540 mg, 1.11 mmol, 1 eq), cyclopropylboronic acid (478.78 mg, 5.57 mmol, 5 eq), SPhos Pd G3 (86.98 mg, 111.48 μmol, 0.1 eq), Cs₂CO₃ (1.5 M, 2.23 mL, 3 eq) in Tol. (6 mL) was degassed and purged with N₂ for 3 times and stirred at 90° C. for 2 hr. The reaction mixture was then cooled and poured into H₂O (10 mL). The resulting mixture was extracted with EtOAc (20 mL*2). The combined organic phases were washed with brine (10 mL), dried over anhydrous sodium sulfate, filtered and concentrated under vacuum. The residue was purified by flash silica gel chromatography (ISCO®; 4 g SepaFlash® Silica Flash Column, eluent 0~20% Ethyl acetate/Petroleum ether gradient @20 mL/min) to afford tert-butyl N-(3-cyclopropyl-6,7-dihydro-5H-thieno[3,2-b]pyran-6-yl)-N-[(2,4-dimethoxyphenyl)methyl]carbamate (4) (500 mg, 785.51 μmol, 70.46% yield) as yellow oil. LC-MS: m/z [M+Na]⁺=468.1

### Procedure for preparation of 3-cyclopropyl-6,7-dihydro-5H-thieno[3,2-b]pyran-6-amine TFA salt (5)

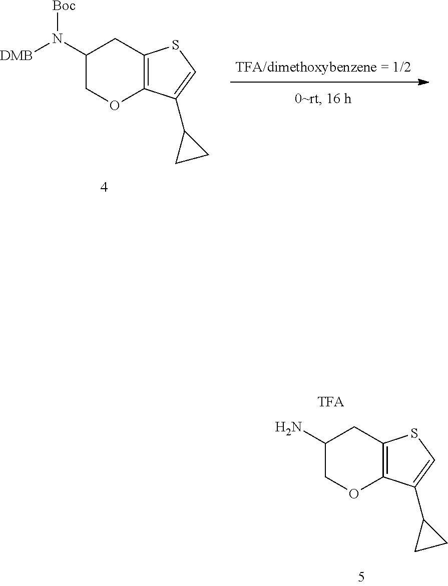

To a solution of tert-butyl N-(3-cyclopropyl-6,7-dihydro-5H-thieno[3,2-b]pyran-6-yl)-N-[(2,4-dimethoxyphenyl)methyl]carbamate (480 mg, 754.09 μmol, 1 eq) in 1,2-dimethoxybenzene (6 mL) was added TFA (4.61 g, 40.39 mmol, 3 mL, 53.56 eq) at 0° C. The mixture was stirred at 25° C. for 16 hr. The reaction mixture was poured into H₂O (20 mL) and extracted with PE (10 mL*2). The organic phase was separated, washed with H₂O 10 mL (10 mL*3). The combined water layer was lyophilized to afford 3-cyclopropyl-6,7-dihydro-5H-thieno[3,2-b]pyran-6-amine TFA salt (140 mg, crude) as a white solid. LC-MS: m/z [M+H]⁺=196.1

### Procedure for preparation of tert-butyl N-(3-cyclopropyl-6,7-dihydro-5H-thieno[3,2-b]pyran-6-yl)carbamate (6)

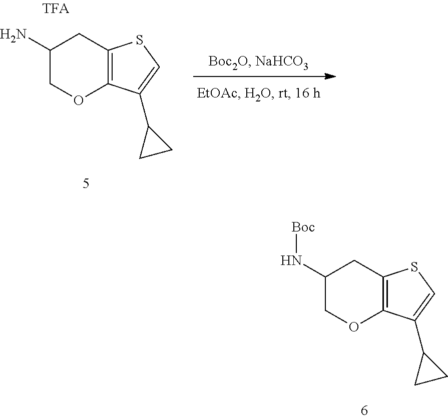 [SMILES: NC1COc2c(C3CC3)csc2C1 ; CC(C)(C)OC(=O)NC1COc2c(C3CC3)csc2C1]

To a solution of 3-cyclopropyl-6,7-dihydro-5H-thieno[3,2-b]pyran-6-amine TFA salt (5) (140 mg, 716.92 μmol, 1 eq) in EtOAc (5 mL) and H₂O (5 mL) was added aq. sat NaHCO₃ (301.13 mg, 3.58 mmol, 139.48 μL, 5 eq) and (Boc)₂O (312.93 mg, 1.43 mmol, 329.40 μL, 2 eq). The mixture was stirred at 25° C. for 16 hr then poured into H₂O (20 mL) and extracted with EtOAc (30 mL*2). The combined organic phases were washed with brine (10 mL), dried over anhydrous sodium sulfate, filtered and concentrated. The residue was purified by flash silica gel chromatography (ISCO®; 4 g SepaFlash® Silica Flash Column, eluent 0~100% Ethyl acetate/Petroleum ether gradient @20 mL/min) and prep-HPLC (column: Waters Xbridge 150*25 mm*5 μm; mobile phase: [water(NH₄HCO₃)-ACN]; gradient:50%-80% B over 9 min) to afford tert-butyl N-(3-cyclopropyl-6,7-dihydro-5H-thieno[3,2-b]pyran-6-yl)carbamate (6) (180 mg, 609.35 μmol, 85.00% yield) as yellow oil. ¹H NMR (400 MHz, CHLOROFORM-d) δ=6.46 (s, 1H), 5.07-4.96 (m, 1H), 4.33-4.03 (m, 3H), 3.07 (dd, J=5.4, 16.3 Hz, 1H), 2.69 (br d, J=15.6 Hz, 1H), 1.84-1.73 (m, 1H), 1.53-1.42 (m, 9H), 0.94-0.81 (m, 2H), 0.73-0.61 (m, 2H).

### Procedure for preparation of tert-butyl N-(3-cyclopropyl-6,7-dihydro-5H-thieno[3,2-b]pyran-6-yl)-N-methyl-carbamate (7)

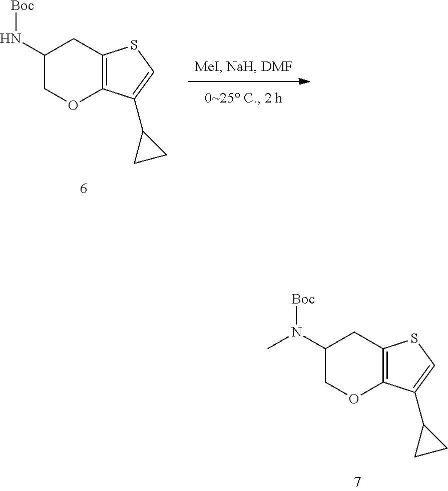 [SMILES: CC(C)(C)OC(=O)NC1COc2c(C3CC3)csc2C1 ; CN(C(=O)OC(C)(C)C)C1COc2c(C3CC3)csc2C1]

To a solution of tert-butyl N-(3-cyclopropyl-6,7-dihydro-5H-thieno[3,2-b]pyran-6-yl)carbamate (6) (120 mg, 406.23 μmol, 1 eq) in DMF (2 mL) was added NaH (32.50 mg, 812.47 μmol, 60% suspension in mineral oil, 2 eq) at 0° C. The mixture was stirred at this temperature for 0.5 hr. Then CH₃I (86.49 mg, 609.35 μmol, 37.93 μL, 1.5 eq) was added dropwise at 0° C. and the resulting mixture was then stirred at 25° C. for 1.5 hr and poured into NH₄Cl aq. saturated solution (10 mL) and extracted with EtOAc (20 mL*2). The combined organic phases were washed with brine (10 mL), dried over anhydrous sodium sulfate, filtered and concentrated. The residue was purified by flash silica gel chromatography (ISCO®; 4 g SepaFlash® Silica Flash Column, eluent 0~17% Ethyl acetate/Petroleum ether gradient @20 mL/min) to afford tert-butyl N-(3-cyclopropyl-6,7-dihydro-5H-thieno[3,2-b]pyran-6-yl)-N-methyl-carbamate (7) (100 mg, 323.18 μmol, 79.56% yield) as a yellow oil. ¹H NMR (400 MHz, CHLOROFORM-d) δ=6.45 (s, 1H), 4.66-4.37 (m, 1H), 4.24-4.06 (m, 2H), 3.01-2.88 (m, 2H), 2.87-2.75 (m, 3H), 1.81-1.71 (m, 1H), 1.49 (s, 9H), 0.93-0.77 (m, 2H), 0.72-0.59 (m, 2H).

### Procedure for preparation of tert-butyl N-(3-cyclopropyl-6,7-dihydro-5H-thieno[3,2-b]pyran-6-yl)-N-methyl-carbamate enantiomer 1 (7a) and enantiomer 2 (7b)

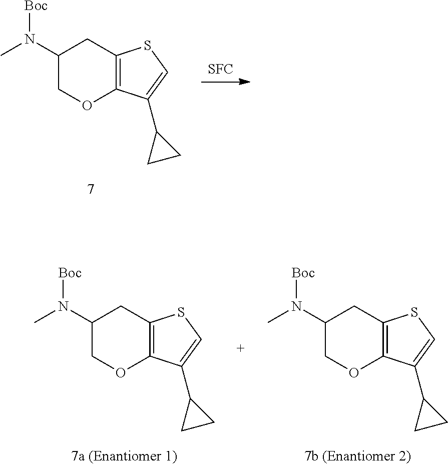

Racemic tert-butyl N-(3-cyclopropyl-6,7-dihydro-5H-thieno[3,2-b]pyran-6-yl)-N-methyl-carbamate (7) was separated to its enantiomers by SFC (column: DAICEL CHIRALPAK IG (250 mm*50 mm, 10 μm); mobile phase: [CO₂-i-PrOH (0.1% NH₃H₂O)]; B %:26%, isocratic elution mode). tert-Butyl N-(3-cyclopropyl-6,7-dihydro-5H-thieno[3,2-b]pyran-6-yl)-N-methyl-carbamate enantiomer 1 (7a) (40 mg, 129.27 μmol, 40.00% yield) and tert-butyl N-(3-cyclopropyl-6,7-dihydro-5H-thieno[3,2-b]pyran-6-yl)-N-methyl-carbamate enantiomer 2 (7b) (40 mg, 129.27 μmol, 40.00% yield) were both isolated as a yellow oil.

### Procedure for preparation of 3-cyclopropyl-N-methyl-6,7-dihydro-5H-thieno[3,2-b]pyran-6-amine hydrochloride enantiomer 1 (324)

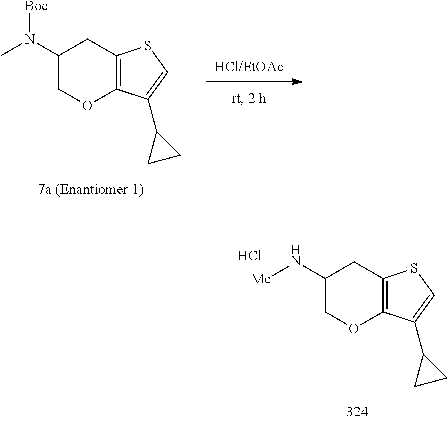 [SMILES: CN(C(=O)OC(C)(C)C)C1COc2c(C3CC3)csc2C1 ; Cl ; CNC1COc2c(C3CC3)csc2C1]

To a solution of tert-butyl N-(3-cyclopropyl-6,7-dihydro-5H-thieno[3,2-b]pyran-6-yl)-N-methyl-carbamate enantiomer 1 (7a) (40 mg, 129.27 μmol, 1 eq) in EtOAc (0.5 mL) was added HCl/EtOAc (2 M, 1.5 mL, 26.52 eq). The mixture was stirred at 25° C. for 2 hr and then concentrated. The crude product was triturated with EtOAc at 25° C. for 10 min then collected and dried to afford 3-cyclopropyl-N-methyl-6,7-dihydro-5H-thieno[3,2-b]pyran-6-amine hydrochloride enantiomer 1 (324) (26.90 mg, 108.36 μmol, 83.82% yield) as a white solid. ¹H NMR (400 MHz, METHANOL-d4) δ=6.67 (s, 1H), 4.56-4.46 (m, 1H), 4.22 (d, J=13.0 Hz, 1H), 3.80 (br s, 1H), 3.28 (br d, J=6.3 Hz, 1H), 2.97 (br d, J=17.6 Hz, 1H), 2.81 (s, 3H), 1.85-1.74 (m, 1H), 0.87-0.79 (m, 2H), 0.70-0.61 (m, 2H). LC-MS: m/z [M+H]⁺=210.0

### Procedure for preparation of 3-cyclopropyl-N-methyl-6,7-dihydro-5H-thieno[3,2-b]pyran-6-amine hydrochloride enantiomer 2 (325)

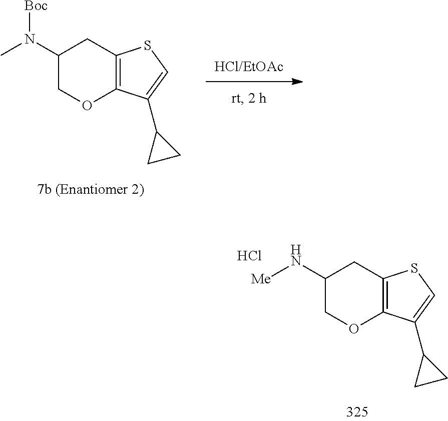 [SMILES: CN(C(=O)OC(C)(C)C)C1COc2c(C3CC3)csc2C1 ; Cl ; CNC1COc2c(C3CC3)csc2C1]

To a solution of tert-butyl N-(3-cyclopropyl-6,7-dihydro-5H-thieno[3,2-b]pyran-6-yl)-N-methyl-carbamate enantiomer 2 (7b)(40 mg, 129.27 μmol, 1 eq) in EtOAc (0.5 mL) was added HCl/EtOAc (2 M, 1.5 mL, 23.21 eq). The mixture was stirred at 25° C. for 2 hr and thenconcentrated. The crude product was triturated with EtOAc at 25° C. for 10 min then collected and dried to afford 3-cyclopropyl-N-methyl-6,7-dihydro-5H-thieno[3,2-b]pyran-6-amine hydrochloride enantiomer 2 (325) (27.86 mg, 112.23 μmol, 86.81% yield) as a white solid. ¹H NMR (400 MHz, METHANOL-d4) δ=6.67 (s, 1H), 4.57-4.49 (m, 1H), 4.22 (d, J=12.4 Hz, 1H), 3.80 (br d, J=1.9 Hz, 1H), 3.28 (br d, J=6.1 Hz, 1H), 3.05-2.92 (m, 1H), 2.81 (s, 3H), 1.87-1.73 (m, 1H), 0.95-0.79 (m, 2H), 0.74-0.62 (m, 2H). LC-MS: m/z [M+H]⁺=210.0

### Synthetic Scheme for preparation of N-methyl-5,6,7,8-tetrahydro-4H-cyclohepta[b]thiophen-5-amine hydrochloride (326) and N-methyl-5,6,7,8-tetrahydro-4H-cyclohepta[b]thiophen-6-amine hydrochloride (327)

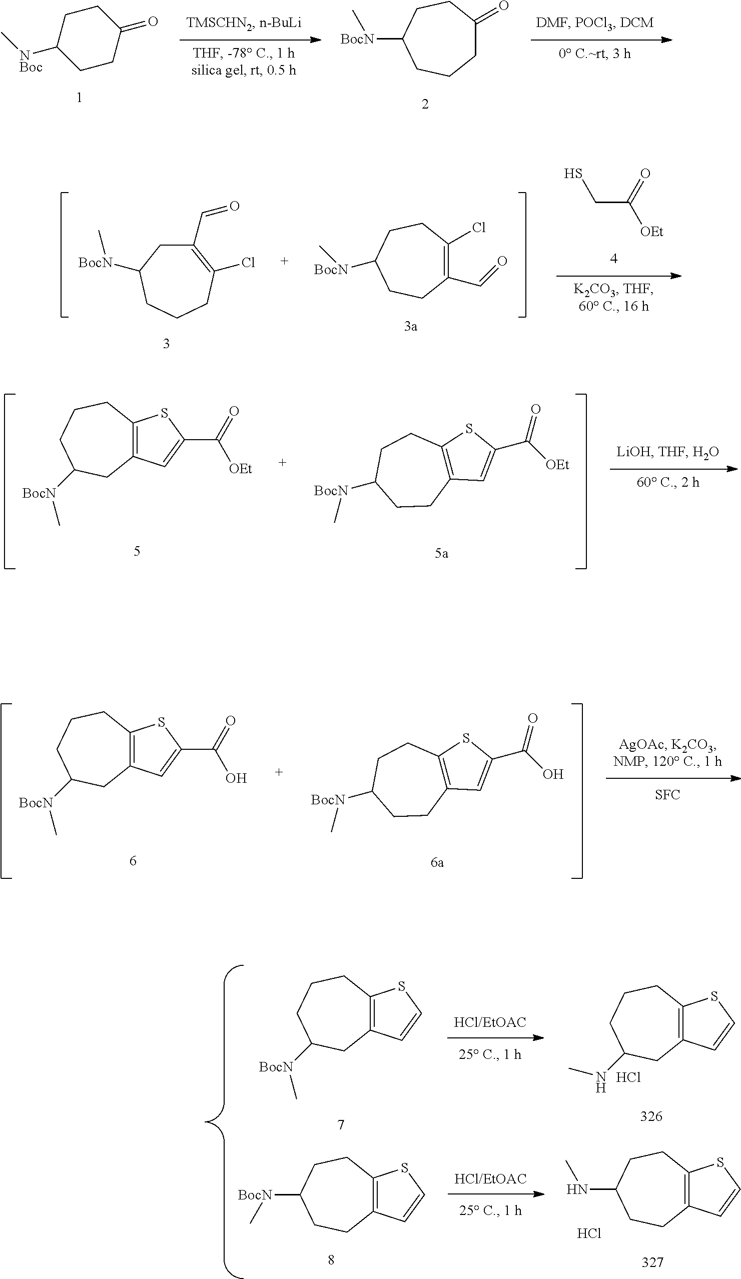

### Procedure for preparation of tert-butyl N-(4-oxocycloheptyl)carbamate (2)

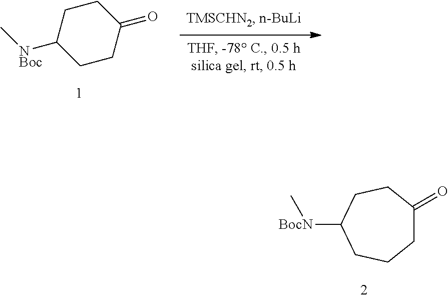 [SMILES: CN(C(=O)OC(C)(C)C)C1CCC(=O)CC1 ; "THF, -78° C., 0.5 hsilica gel, rt, 0.5 h" ; CN(C(=O)OC(C)(C)C)C1CCCC(=O)CC1]

To a solution of n-BuLi (2.5 M, 24.38 mL, 1.3 eq) in THF (50 mL) was added TMSCHN₂ (2 M, 30.48 mL, 1.3 eq). The mixture was stirred at −78° C. for 0.1 h. Then tert-butyl N-(4-oxocyclohexyl)carbamate (1) (10 g, 46.89 mmol, 10.00 mL, 1 eq) in THF (50 mL) was added to the mixture. The mixture was stirred at −78° C. for 0.5 h. The reaction mixture was quenched by addition MeOH (0.4 m)L at 25° C., extracted with MTBE 200 mL (100 mL*2). The combined organic layers were washed with NaCl aq. sat. (100 mL*2), dried over Na₂SO₄ and filtered. To the filtrate was added 20 g silica gel and 20 g Na₂SO₄. The mixture was stirred at 25° C. for 0.5 h then filtered and concentrated under reduced pressure. The residue was purified by flash silica gel chromatography (ISCO®; 120 g SepaFlash® Silica Flash Column, eluent 0~20% Ethyl acetate/Petroleum ether gradient @60 mL/min). Compound tert-butyl N-(4-oxocycloheptyl)carbamate (2) (8.3 g, 36.52 mmol, 77.88% yield) was obtained as a white solid. ¹H NMR (400 MHz, CHLOROFORM-d) δ=4.55 (br s, 1H), 3.96-3.58 (m, 1H), 2.56-2.39 (m, 4H), 2.28-2.07 (m, 2H), 1.93-1.79 (m, 1H), 1.70-1.53 (m, 2H), 1.45 (d, J=5.6 Hz, 9H).

### Procedure for preparation of tert-butyl N-(4-chloro-3-formyl-cyclohept-3-en-1-yl)-N-methyl-carbamate (3) and tert-butyl N-(4-chloro-5-formyl-cyclohept-4-en-1-yl)-N-methyl-carbamate (3a)

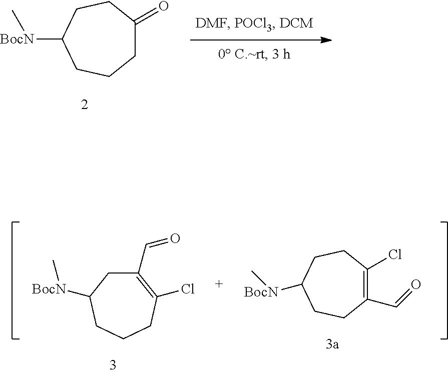

To a solution of DMF (1.82 g, 24.86 mmol, 1.91 mL, 6 eq) in DCM (10 mL) was added POCl₃ (3.18 g, 20.72 mmol, 1.93 mL, 5 eq) dropwise at 0° C. The mixture was stirred at 25° C. for 1 h, and then a solution of tert-butyl N-methyl-N-(4-oxocycloheptyl)carbamate (1 g, 4.14 mmol, 1 eq) in DCM (10 mL) was added dropwise at 0° C. The resulting mixture was stirred at 25° C. for 2 h quenched with aq. sat. NaHCO₃ (100 mL) and extracted with EtOAc (50 mL*2). The combined organic layers were washed with aq. sat. NaCl (10 mL*2), dried over Na₂SO₄, filtered and concentrated under reduced pressure. The residue was purified by flash silica gel chromatography (ISCO®; 40 g SepaFlash® Silica Flash Column, eluent 0~20% Ethyl acetate/Petroleum ether gradient @50 mL/min) to afford a mixture of tert-butyl N-(4-chloro-3-formyl-cyclohept-3-en-1-yl)-N-methyl-carbamate (3) and tert-butyl N-(4-chloro-5-formyl-cyclohept-4-en-1-yl)-N-methyl-carbamate (3A) (1 g, 3.47 mmol, 83.86% yield) as a white solid.

### Procedure for preparation of ethyl 5-[tert-butoxycarbonyl(methyl)amino]-5,6,7,8-tetrahydro-4H-cyclohepta[b]thiophene-2-carboxylate (5) and ethyl 6-[tert-butoxycarbonyl(methyl)amino]-5,6,7,8-tetrahydro-4H-cyclohepta[b]thiophene-2-carboxylate (5a)

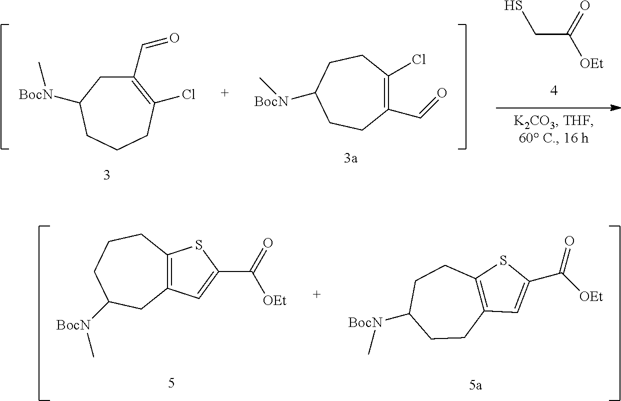

To a mixture of tert-butyl N-(4-chloro-3-formyl-cyclohept-3-en-1-yl)-N-methyl-carbamate (3) and tert-butyl N-(4-chloro-5-formyl-cyclohept-4-en-1-yl)-N-methyl-carbamate (3a) (1.00 g, 3.47 mmol, 1 eq) in EtOH (20 mL) was added K₂CO₃ (720.39 mg, 5.21 mmol, 1.5 eq) and ethyl 2-sulfanylacetate (501.09 mg, 4.17 mmol, 457.20 μL, 1.2 eq). The resulting mixture was stirred at 60° C. for 16 hr then cooled and concentrated under reduced pressure. Followed dilution with H₂O (300 mL) and extraction with EtOAc (200 mL*2). The combined organic layers were washed with brine (200 mL), dried over anhydrous Na₂SO₄, filtered and concentrated under reduced pressure to give a residue that was purified by flash silica gel chromatography (ISCO®; 40 g SepaFlash® Silica Flash Column, eluent 0~20% Ethyl acetate/Petroleum ether gradient @60 mL/min). This afforded a mixture of ethyl 5-[tert-butoxycarbonyl(methyl)amino]-5,6,7,8-tetrahydro-4H-cyclohepta[b]thiophene-2-carboxylate (5) and ethyl 6-[tert-butoxycarbonyl(methyl)amino]-5,6,7,8-tetrahydro-4H-cyclohepta[b]thiophene-2-carboxylate (5a) (1.2 g, 3.39 mmol, 97.70% yield) as a white solid. LC-MS: m/z [M+H-tBu]⁺=298.1

### Procedure for preparation of 5-[tert-butoxycarbonyl(methyl)amino]-5,6,7,8-tetrahydro-4H-cyclohepta[b]thiophene-2-carboxylic acid (6) and 6-[tert-butoxycarbonyl(methyl)amino]-5,6,7,8-tetrahydro-4H-cyclohepta[b]thiophene-2-carboxylic acid (6a)

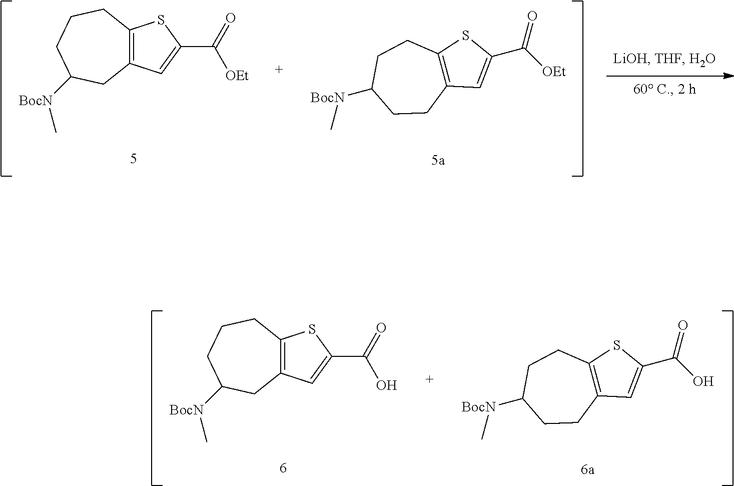

A solution of ethyl 6-[tert-butoxycarbonyl(methyl)amino]-5,6,7,8-tetrahydro-4H-cyclohepta[b]thiophene-2-carboxylate (5) and ethyl 5-[tert-butoxycarbonyl(methyl)amino]-5,6,7,8-tetrahydro-4H-cyclohepta[b]thiophene-2-carboxylate (5a) (1.20 g, 3.39 mmol, 1 eq) in THF (20 mL) and H₂O (20 mL) was treated with LiOH·H₂O (284.92 mg, 6.79 mmol, 2 eq). The resulting mixture was stirred at 60° C. for 2 hr then cooled and concentrated under reduced pressure to remove THF. The mixture was then acidified to pH=6 with 1 M HCl aqueous solution. The precipitate formed was collected by filtration and air-dried.to afford a mixture of 5-[tert-butoxycarbonyl(methyl)amino]-5,6,7,8-tetrahydro-4H-cyclohepta[b]thiophene-2-carboxylic acid (6) and 6-[tert-butoxycarbonyl(methyl)amino]-5,6,7,8-tetrahydro-4H-cyclohepta[b]thiophene-2-carboxylic acid (6a) (700 mg, 2.15 mmol, 63.36% yield) was obtained as a white solid. LC-MS: m/z [M+H-tBu]⁺=270.0

### Procedure for preparation of tert-butyl N-methyl-N-(5,6,7,8-tetrahydro-4H-cyclohepta[b]thiophen-5-yl)carbamate (7) and tert-butyl N-methyl-N-(5,6,7,8-tetrahydro-4H-cyclohepta[b]thiophen-6-yl)carbamate (8)

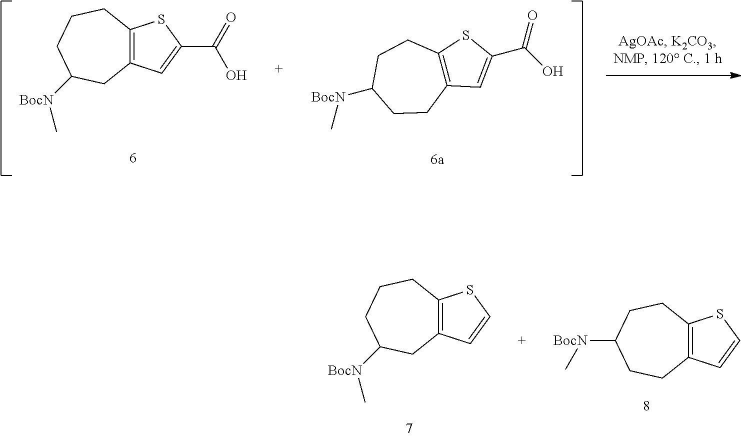

To a solution of 5-[tert-butoxycarbonyl(methyl)amino]-5,6,7,8-tetrahydro-4H-cyclohepta[b]thiophene-2-carboxylic acid (6) and 6-[tert-butoxycarbonyl(methyl)amino]-5,6,7,8-tetrahydro-4H-cyclohepta[b]thiophene-2-carboxylic acid (6a) (700 mg, 2.15 mmol, 1 eq) in NMP (6 mL) was added AgOAc (179.52 mg, 1.08 mmol, 0.5 eq) and K₂CO₃ (891.86 mg, 6.45 mmol, 3 eq). The mixture was stirred at 120° C. for 2 hr then cooled, filtered and the filtrate was diluted with H₂O (200 ml) and extracted with EtOAc (50 mL*2). The combined organic layers were washed with aq. sat. NaCl (50 mL*2), dried over Na₂SO₄, filtered and concentrated under reduced pressure. The residue was purified by prep-HPLC (FA condition:column: Phenomenex luna C18 150*40 mm*15 μm; mobile phase: [water(FA)-ACN]; gradient:54%-84% B over 15 min) and by SFC (column: DAICEL CHIRALPAK AD (250 mm*50 mm, 10 μm); mobile phase: [Hexane-IPA (0.1% NH₃)]; B %:10%, isocratic elution mode) to afford the desired compounds as racemates. Compound tert-butyl N-methyl-N-(5,6,7,8-tetrahydro-4H-cyclohepta[b]thiophen-5-yl)carbamate (7) (ca. 200 mg, 700 μmol, 30% yield) was obtained as a white solid. Compound tert-butyl N-methyl-N-(5,6,7,8-tetrahydro-4H-cyclohepta[b]thiophen-6-yl)carbamate (8) (ca. 200 mg, 700 μmol, 30% yield) was obtained as a white solid.

### Procedure for preparation of N-methyl-5,6,7,8-tetrahydro-4H-cyclohepta[b]thiophen-5-amine hydrochloride (326)

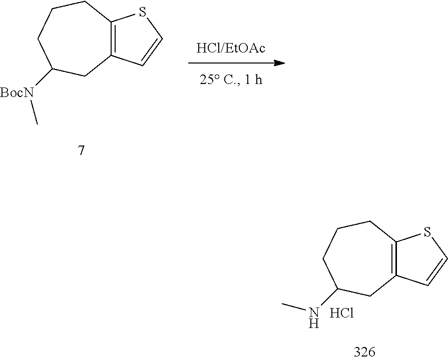 [SMILES: CN(C(=O)OC(C)(C)C)C1CCCc2sccc2C1 ; CNC1CCCc2sccc2C1 ; Cl]

To a solution of tert-butyl N-methyl-N-(5,6,7,8-tetrahydro-4H-cyclohepta[b]thiophen-5-yl)carbamate (7) (100 mg, 355.35 μmol, 1 eq) in EtOAc (2 mL) was added HCl/EtOAc (2 M, 4 mL, 22.51 eq). The mixture was stirred at 25° C. for 1 hr and then concentrated. The residue was purified by prep-HPLC (HCl condition:column: Phenomenex luna C18 150*25 mm*10 μm; mobile phase: [water(HCl)-ACN]; gradient:1%-30% B over 10 min). Compound N-methyl-5,6,7,8-tetrahydro-4H-cyclohepta[b]thiophen-5-amine hydrochloride (326) (38.45 mg, 175.94 μmol, 49.51% yield) was obtained as a white solid. ¹H NMR (400 MHz, METHANOL-d₄) δ=7.07 (d, J=5.1 Hz, 1H), 6.89 (d, J=5.1 Hz, 1H), 3.28-3.16 (m, 2H), 3.06-2.83 (m, 3H), 2.74 (s, 3H), 2.37-2.28 (m, 1H), 2.10-1.92 (m, 2H), 1.77-1.66 (m, 1H). LC-MS: m/z [M+H]⁺=182.1.

### Procedure for preparation of N-methyl-5,6,7,8-tetrahydro-4H-cyclohepta[b]thiophen-6-amine hydrochloride (327)

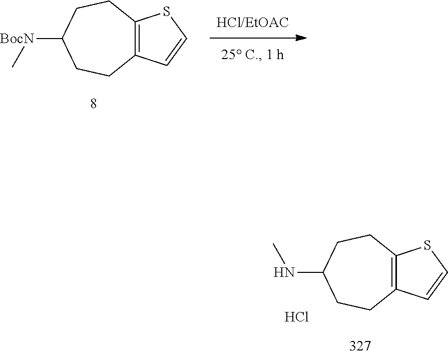 [SMILES: CN(C(=O)OC(C)(C)C)C1CCc2ccsc2CC1 ; CNC1CCc2ccsc2CC1 ; Cl]

To a solution of tert-butyl N-methyl-N-(5,6,7,8-tetrahydro-4H-cyclohepta[b]thiophen-6-yl)carbamate (8) (100.00 mg, 355.35 μmol, 1 eq) in EtOAc (2 mL) was added HCl/EtOAc (2 M, 4 mL, 22.51 eq). The mixture was stirred at 25° C. for 1 hr and then concentrated under reduced pressure to remove EtOAc. The residue was purified by prep-HPLC (HCl condition:column: Phenomenex luna C18 150*25 mm*10 μm; mobile phase: [water(HCl)-ACN]; gradient:1%-30% B over 10 min). Compound N-methyl-5,6,7,8-tetrahydro-4H-cyclohepta[b]thiophen-6-amine hydrochloride (327) (19.09 mg, 87.67 μmol, 24.67% yield was obtained as a white solid. ¹H NMR (400 MHz, METHANOL-d₄) δ=7.03 (d, J=5.1 Hz, 1H), 6.83 (d, J=5.1 Hz, 1H), 3.42-3.36 (m, 1H), 3.06 (dddd, J=2.1, 7.0, 13.3, 15.6 Hz, 2H), 2.87-2.79 (m, 1H), 2.76 (s, 3H), 2.69-2.60 (m, 1H), 2.36-2.23 (m, 2H), 1.67-1.50 (m, 2H). LC-MS: m/z [M+H]⁺=182.1

### Synthetic scheme for preparation of N, 5-dimethyl-5,6-dihydro-4H-cyclopenta[b]thiophen-5-amine hydrochloride (328)

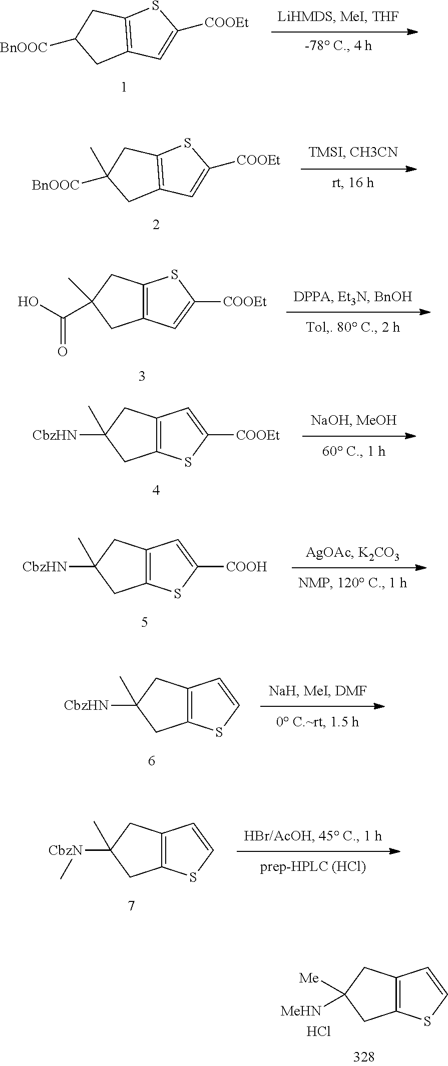

### Procedure for preparation of 5-benzyl 2-ethyl 5-methyl-5,6-dihydro-4H-cyclopenta[b]thiophene-2,5-dicarboxylate (2)

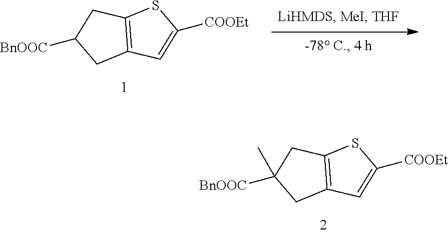

To a solution of LiHMDS (1 M, 39.35 mL, 1.3 eq) in THF (60 mL) was added 5-benzyl 2-ethyl 5,6-dihydro-4H-cyclopenta[b]thiophene-2,5-dicarboxylate (1) (10 g, 30.27 mmol, 1 eq) in THF (20 mL) dropwise at −78° C. and then after 1 h MeI (5.58 g, 39.35 mmol, 2.45 mL, 1.3 eq). The mixture was stirred at −78° C. for 2 hr under N₂. The reaction mixture was quenched by addition of sat. aq. NH₄Cl 200 mL at 0° C., and then extracted with EtOAc (100 mL*2). The combined organic layers were washed with brine (100 mL), dried over anhydrous Na₂SO₄, filtered and concentrated under reduced pressure. The residue was purified by flash silica gel chromatography (ISCO®; 20 g SepaFlash® Silica Flash Column, eluent 0~10% Ethyl acetate/Petroleum ether gradient @40 mL/min). Compound 5-benzyl 2-ethyl 5-methyl-4,6-dihydrocyclopenta[b]thiophene-2,5-dicarboxylate (2) (8.5 g, 24.68 mmol, 81.54% yield) was obtained as a yellow oil. ¹H NMR (400 MHz, CHLOROFORM-d) δ=7.51 (s, 1H), 7.37-7.32 (m, 5H), 5.18 (s, 2H), 4.33 (q, J=7.1 Hz, 2H), 3.54 (d, J=16.6 Hz, 1H), 3.34 (d, J=15.4 Hz, 1H), 2.84 (d, J=16.6 Hz, 1H), 2.69 (d, J=15.5 Hz, 1H), 1.49 (s, 3H), 1.38-1.34 (m, 3H)

### Procedure for preparation of 2-(ethoxycarbonyl)-5-methyl-5,6-dihydro-4H-cyclopenta[b]thiophene-5-carboxylic acid (3)

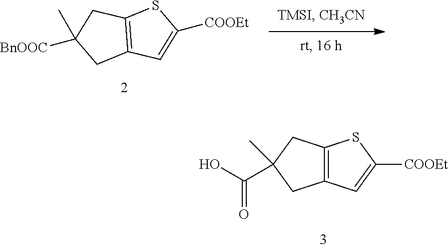

To a solution of 5-benzyl 2-ethyl 5-methyl-4,6-dihydrocyclopenta[b]thiophene-2,5-dicarboxylate (2) (8 g, 23.23 mmol, 1 eq) in ACN (80 mL) was added TMSI (23.24 g, 116.14 mmol, 15.81 mL, 5 eq) at 0° C. The mixture was stirred at 25° C. for 16 hr and then concentrated under reduced pressure. The residue was purified by flash silica gel chromatography (ISCO®; 40 g SepaFlash® Silica Flash Column, eluent 0-20% Ethyl acetate/Petroleum ether gradient @40 mL/min). Compound 2-ethoxycarbonyl-5-methyl-4,6-dihydrocyclopenta[b]thiophene-5-carboxylic acid (3) (5 g, 19.66 mmol, 84.65% yield) was obtained as a brown oil.

### Procedure for preparation of ethyl 5-(((benzyloxy)carbonyl)amino)-5-methyl-5,6-dihydro-4H-cyclopenta[b]thiophene-2-carboxylate (4)

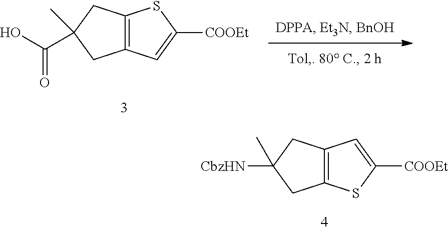 [SMILES: CCOC(=O)c1cc2c(s1)CC(C)(C(=O)O)C2 ; CCOC(=O)c1cc2c(s1)CC(C)(NC(=O)OCc1ccccc1)C2]

To a solution of 2-ethoxycarbonyl-5-methyl-4,6-dihydrocyclopenta[b]thiophene-5-carboxylic acid (3) (5 g, 19.66 mmol, 1 eq) and BnOH (4.25 g, 39.32 mmol, 4.07 mL, 2 eq) in Tol. (60 mL) was added DPPA (6.49 g, 23.59 mmol, 5.09 mL, 1.2 eq) and Et₃N (3.98 g, 39.32 mmol, 5.47 mL, 2 eq). The mixture was stirred at 80° C. for 2 h under N₂ then cooled and washed with aq. sat. NaHCO₃ (100 mL). The aq. layer was then extracted with EtOAc (100 mL*2) and the combined organic layers were washed with aq. sat. NaCl (100 mL), dried over anhydrous Na₂SO₄, filtered and concentrated under reduced pressure to give a residue. The residue was purified by flash silica gel chromatography (ISCO®; 40 g SepaFlash® Silica Flash Column, eluent 0~20% Ethyl acetate/Petroleum ether gradient @40 mL/min). Compound ethyl 5-(benzyloxycarbonylamino)-5-methyl-4,6-dihydrocyclopenta[b]thiophene-2-carboxylate (4) (6 g, 16.69 mmol, 84.90% yield) was obtained as a yellow oil. ¹H NMR (400 MHz, CHLOROFORM-d) δ=7.51 (s, 1H), 7.38 (d, J=4.4 Hz, 5H), 5.08 (s, 2H), 4.36-4.30 (m, 2H), 3.43 (br d, J=16.3 Hz, 1H), 3.20 (br d, J=15.0 Hz, 1H), 2.94 (d, J=16.4 Hz, 1H), 2.77 (d, J=15.3 Hz, 1H), 1.54 (s, 3H), 1.38-1.34 (m, 3H).

### Procedure for preparation of 5-(benzyloxycarbonylamino)-5-methyl-5,6-dihydro-4H-cyclopenta[b]thiophene-2-carboxylic acid (5)

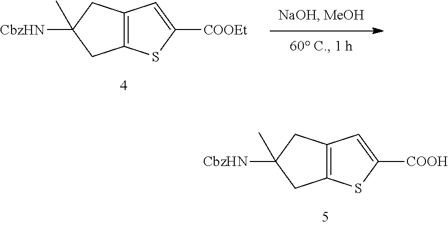 [SMILES: CCOC(=O)c1cc2c(s1)CC(C)(NC(=O)OCc1ccccc1)C2 ; CC1(NC(=O)OCc2ccccc2)Cc2cc(C(=O)O)sc2C1]

To a solution of ethyl 5-(benzyloxycarbonylamino)-5-methyl-4,6-dihydrocyclopenta[b]thiophene-2-carboxylate (4) (5 g, 13.91 mmol, 1 eq) in EtOH (40 mL) was added NaOH (1.67 g, 41.73 mmol, 3 eq) in H₂O (10 mL). The mixture was stirred at 60° C. for 1 hr. The reaction mixture was then concentrated under reduced pressure to remove EtOH then added H₂O (20 mL) and the mixture pH was adjusted to 4 with 1M HCl. The resulting mixture was extracted with EtOAc (30 mL*3). The combined organic layers were washed with saturated aq. NaCl (50 mL), dried over anhydrous Na₂SO₄, filtered and concentrated under reduced pressure. The residue was purified by flash silica gel chromatography (ISCO®; 80 g SepaFlash® Silica Flash Column, eluent 0-29% Ethylacetate/Petroleum ether gradient @100 mL/min). Compound 5-(benzyloxycarbonylamino)-5-methyl-4,6-dihydrocyclopenta[b]thiophene-2-carboxylic acid (5) (3.4 g, 9.85 mmol, 70.81% yield) was obtained as a light yellow solid. ¹H NMR (400 MHz, CHLOROFORM-d) δ=7.59 (s, 1H), 7.35 (br d, J=3.8 Hz, 5H), 5.09 (br s, 2H), 3.48 (br d, J=16.9 Hz, 1H), 3.31-3.19 (m, 1H), 3.17-3.06 (m, 1H), 2.96 (d, J=16.6 Hz, 1H), 2.79 (d, J=15.2 Hz, 1H), 2.52 (td, J=6.8, 13.5 Hz, 1H), 1.55 (s, 3H)

### Procedure for preparation of benzyl (5-methyl-5,6-dihydro-4H-cyclopenta[b]thiophen-5-yl)carbamate (6)

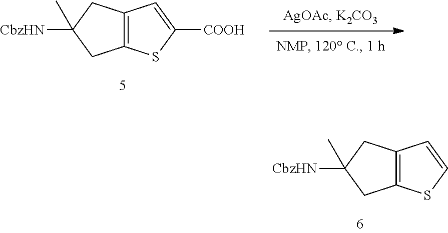 [SMILES: CC1(NC(=O)OCc2ccccc2)Cc2cc(C(=O)O)sc2C1 ; CC1(NC(=O)OCc2ccccc2)Cc2ccsc2C1]

To a solution of 5-(benzyloxycarbonylamino)-5-methyl-4,6-dihydrocyclopenta[b]thiophene-2-carboxylic acid (5) (3.4 g, 10.26 mmol, 1 eq) in NMP (40 mL) was added AgOAc (171.25 mg, 1.03 mmol, 0.1 eq) and K₂CO₃ (4.25 g, 30.78 mmol, 3 eq). The mixture was stirred at 120° C. for 1 hr under N₂ atmosphere, then cooled and poured into H₂O (100 mL). The pH of the mixture was adjusted to 3~4 with 1M aq. HCl. Followed extraction with EtOAc (30 mL*3). The combined organic layers were washed with brine (50 mL*2), dried over anhydrous Na₂SO₄, filtered and concentrated under reduced pressure. The residue was purified by flash silica gel chromatography (ISCO®; 40 g SepaFlash® Silica Flash Column, eluent 0~15% Ethyl acetate/Petroleum ether gradient @60 mL/min). Compound benzyl N-(5-methyl-4,6-dihydrocyclopenta[b]thiophen-5-yl)carbamate (6) (1.6 g, 5.40 mmol, 52.64% yield) was obtained as a colorless oil. ¹H NMR (400 MHz, CHLOROFORM-d) δ=7.38-7.29 (m, 5H), 7.20-7.13 (m, 1H), 6.79 (d, J=4.9 Hz, 1H), 5.09 (s, 2H), 3.33 (br d, J=15.6 Hz, 1H), 3.14 (br d, J=15.0 Hz, 1H), 2.95 (d, J=15.6 Hz, 1H), 2.81 (d, J=15.1 Hz, 1H), 1.59 (d, J=6.6 Hz, 3H).

### Procedure for the preparation of benzyl methyl(5-methyl-5,6-dihydro-4H-cyclopenta[b]thiophen-5-yl)carbamate (7)

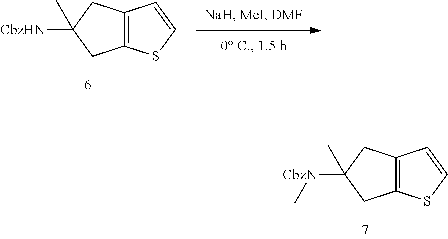 [SMILES: CC1(NC(=O)OCc2ccccc2)Cc2ccsc2C1 ; CN(C(=O)OCc1ccccc1)C1(C)Cc2ccsc2C1]

To a solution of benzyl N-(5-methyl-4,6-dihydrocyclopenta[b]thiophen-5-yl)carbamate (800 mg, 2.78 mmol, 1 eq) in DMF (10 mL) was added in portions NaH (145.00 mg, 3.63 mmol, 60% suspension in mineral oil, 1.3 eq) at 0° C. The mixture was stirred for 30 min, and then MeI (513.00 mg, 3.61 mmol, 225.00 μL, 1.3 eq) in DMF (0.5 mL) was added dropwise at 0° C. After stirring at 0° C. for 1 hr, the reaction mixture was quenched with aq. sat NH₄Cl (50 mL) and then extracted with EtOAc (20 mL*2). The combined organic layers were washed with brine (30 mL*2), dried over anhydrous Na₂SO₄, filtered and concentrated under reduced pressure. The residue was purified by prep-HPLC (neutral condition: column: Waters Xbridge 150*25 mm*5 μm; mobile phase: [water(NH₄HCO₃)-ACN]; gradient:58%-88% B over 9 min). Compound benzyl N-methyl-N-(5-methyl-4,6-dihydrocyclopenta[b]thiophen-5-yl)carbamate (7) (350 mg, 1.16 mmol, 41.71% yield, 100% purity) was obtained as a colorless oil. ¹H NMR (400 MHz, CHLOROFORM-d) 6=7.39-7.30 (m, 5H), 7.14 (d, J=4.9 Hz, 1H), 6.79 (d, J=4.9 Hz, 1H), 5.15 (s, 2H), 3.33 (br d, J=15.3 Hz, 1H), 3.23-3.08 (m, 2H), 3.03 (s, 3H), 2.92 (d, J=14.6 Hz, 1H), 1.43 (s, 3H)

### Procedure for preparation of N, 5-dimethyl-5,6-dihydro-4H-cyclopenta[b]thiophen-5-amine hydrochloride (328)

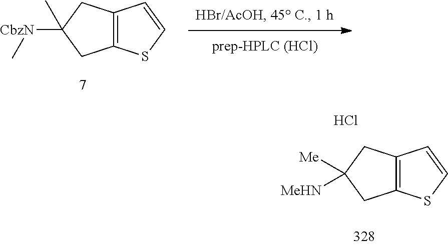 [SMILES: CN(C(=O)OCc1ccccc1)C1(C)Cc2ccsc2C1 ; Cl ; CNC1(C)Cc2ccsc2C1]

A mixture of benzyl N-methyl-N-(5-methyl-4,6-dihydrocyclopenta[b]thiophen-5-yl)carbamate (7) (100 mg, 331.78 μmol, 1 eq) in AcOH (1 mL) and 33% HBr in acetic acid (745.00 mg, 3.22 mmol, 0.5 mL, 9.71 eq) was stirred at 45° C. for 1 h and then concentrated under vacuum. The residue was dissolved with MeOH (2 mL) and the mixture was neutralized with solid NaHCO₃ and then filtered and purified by prep-HPLC (HCl condition; column: Phenomenex luna C18 150*25 mm*10 μm; mobile phase: [water(HCl)-ACN]; gradient:1%-20% B over 1 min). Compound N, 5-dimethyl-4,6-dihydrocyclopenta[b]thiophen-5-amine hydrochloride (328) (11.80 mg, 57.92 μmol, 17.46% yield) was obtained as a white solid. ¹H NMR (400 MHz, METHANOL-d₄) δ=7.36 (d, J=5.0 Hz, 1H), 6.87 (d, J=5.0 Hz, 1H), 3.29-3.23 (m, 1H), 3.16-3.08 (m, 2H), 3.03-2.95 (m, 1H), 2.69 (s, 3H), 1.62 (s, 3H). LC-MS: m/z [M+H]⁺=168.1

### Synthetic scheme for preparation of 3-cyclopropyl-6,7-dihydro-5H-thieno[3,2-b]pyran-6-amine hydrochloride enantiomer 1 (329) and enantiomer 2 (330)

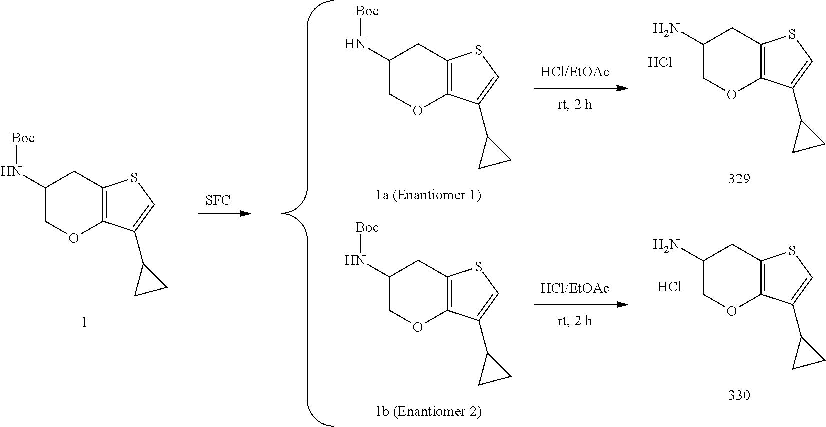

### Procedure for preparation of tert-butyl N-(3-cyclopropyl-6,7-dihydro-5H-thieno[3,2-b]pyran-6-yl)carbamate enantiomer 1 (1a) and enantiomer 2 (1b)

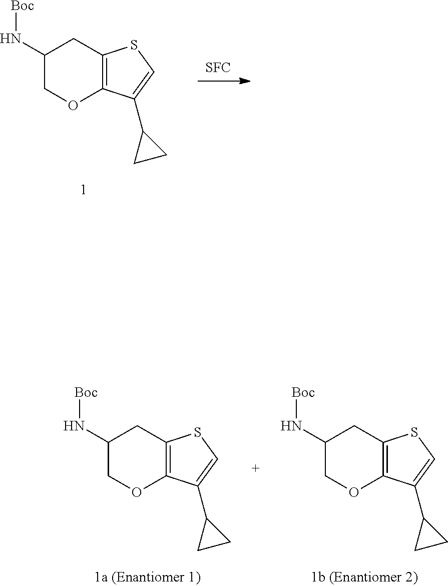

Racemic tert-butyl N-(3-cyclopropyl-6,7-dihydro-5H-thieno[3,2-b]pyran-6-yl)carbamate (1) (60 mg) was separated by SFC (column: DAICEL CHIRALPAK AD (250 mm*30 mm, 10 μm); mobile phase: [CO₂-MeOH (0.1% NH₃H₂O)]; B %:30%, isocratic elution mode) to afford tert-butyl N-(3-cyclopropyl-6,7-dihydro-5H-thieno[3,2-b]pyran-6-yl)carbamate enantiomer 1 (1a) (25 mg, 84.63 μmol, 41.67% yield) and tert-butyl N-(3-cyclopropyl-6,7-dihydro-5H-thieno[3,2-b]pyran-6-yl)carbamate enantiomer 2 (1b) (25 mg, 84.63 μmol, 41.67% yield) both as yellow oils.

### Procedure for preparation of 3-cyclopropyl-6,7-dihydro-5H-thieno[3,2-b]pyran-6-amine hydrochloride enantiomer 1 (329)

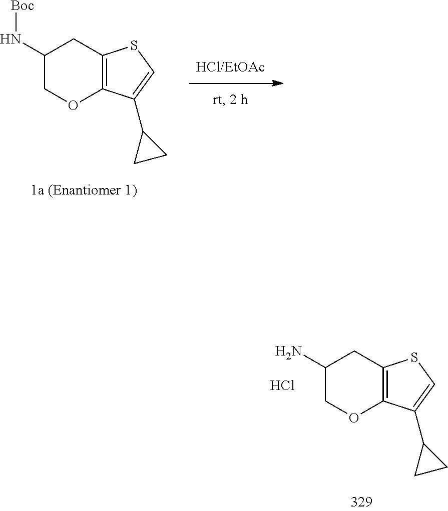 [SMILES: CC(C)(C)OC(=O)NC1COc2c(C3CC3)csc2C1 ; Cl ; NC1COc2c(C3CC3)csc2C1]

To a solution of tert-butyl N-(3-cyclopropyl-6,7-dihydro-5H-thieno[3,2-b]pyran-6-yl)carbamate enantiomer 1 (1a) (25 mg, 84.63 μmol, 1 eq) in EtOAc (0.5 mL) was added HCl/EtOAc (2 M, 1.5 mL, 35.45 eq). The mixture was stirred at 25° C. for 2 hr and then concentrated. The residue was purified by prep-HPLC (column: Phenomenex luna C18 150*25 mm*10 μm; mobile phase: [water(HCl)-ACN]; gradient:1%-30% B over 10 min) to afford 3-cyclopropyl-6,7-dihydro-5H-thieno[3,2-b]pyran-6-amine hydrochloride enantiomer 1 (329) (9.31 mg, 39.37 μmol, 46.52% yield) as a white solid. 1H NMR (400 MHz, METHANOL-d4) δ=6.66 (s, 1H), 4.41-4.30 (m, 1H), 4.26-4.16 (m, 1H), 3.97-3.85 (m, 1H), 3.29-3.24 (m, 1H), 2.83 (br d, J=17.1 Hz, 1H), 1.79 (tt, J=5.2, 8.4 Hz, 1H), 0.91-0.79 (m, 2H), 0.67 (dtd, J=1.7, 3.7, 6.9 Hz, 2H). LC-MS: m/z [M+H]⁺=196.0.

### Procedure for preparation of 3-cyclopropyl-6,7-dihydro-5H-thieno[3,2-b]pyran-6-amine hydrochloride enantiomer 2 (330)

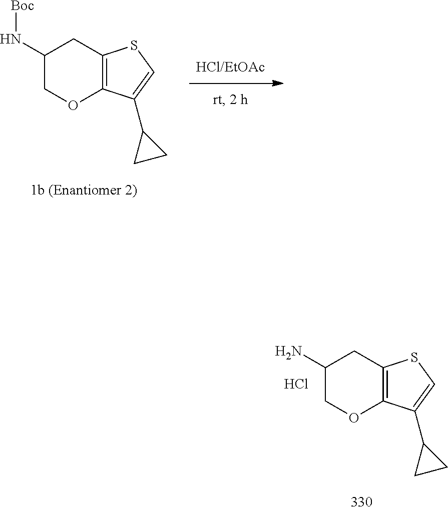 [SMILES: CC(C)(C)OC(=O)NC1COc2c(C3CC3)csc2C1 ; Cl ; NC1COc2c(C3CC3)csc2C1]

To a solution of tert-butyl N-(3-cyclopropyl-6,7-dihydro-5H-thieno[3,2-b]pyran-6-yl)carbamate enantiomer 2 (1b) (25 mg, 84.63 μmol, 1 eq) in EtOAc (0.5 mL) was added HCl/EtOAc (2 M, 1.5 mL, 35.45 eq). The mixture was stirred at 25° C. for 2 hr and then concentrated. The residue was purified by prep-HPLC (column: Phenomenex luna C18 150*25 mm*10 μm; mobile phase: [water(HCl)-ACN]; gradient:1%-30% B over 10 min) to afford 3-cyclopropyl-6,7-dihydro-5H-thieno[3,2-b]pyran-6-amine hydrochloride enantiomer 2 (330) (11.54 mg, 48.80 μmol, 57.66% yield) as a white solid. ¹H NMR (400 MHz, METHANOL-d4) δ=6.66 (s, 1H), 4.38-4.29 (m, 1H), 4.20 (dd, J=1.3, 12.0 Hz, 1H), 3.93-3.86 (m, 1H), 3.26 (d, J=6.0 Hz, 1H), 2.83 (br d, J=17.4 Hz, 1H), 1.79 (tt, J=5.2, 8.4 Hz, 1H), 0.89-0.78 (m, 2H), 0.72-0.58 (m, 2H). LC-MS: m/z [M+H]⁺=196.0

### Biological Activity Assays

### Neuropharmacological Assay (SmartCube™)

To demonstrate the utility of the provided compounds to treat neurological and psychiatric diseases and disorders, exemplary compounds are evaluated using the neuropharmacological screen described in S. L. Roberds et al. Front. Neurosci. 2011 Sep. 9; 5: 103 (doi: 10.3389/fnins.2011.00103) (“Roberds”). As reported in Roberds, because psychiatric diseases generally result from disorders of cell-cell communication or circuitry, intact systems are useful in detecting improvement in disease-relevant endpoints. These endpoints are typically behavioral in nature, often requiring human observation and interpretation. To facilitate testing of multiple compounds for behavioral effects relevant to psychiatric disease, PsychoGenics, Inc. (Tarrytown, NY, “PGI”) developed SmartCube™ an automated system in which behaviors of compound-treated mice are captured by digital video and analyzed with computer algorithms. (D. Brunner et al. Drug Discov. Today 2002, 7:S107-S112). PGI Analytical Systems uses data from SmartCube™ to compare the behavioral signature of a test compound to a database of behavioral signatures obtained using a large set of diverse reference compounds. (The composition of the database as well as validation of the method is further described in Roberds). In this way, the neuropharmacological effects of a test compound can be predicted by similarity to major classes of compounds, such as antipsychotics, anxiolytics and antidepressants.

The SmartCube™ system produces an activity signature indicating the probability that the activity of the test compound at the administered dose matches a given class of neuropharmacological agents. (See, e.g., Roberds, FIGS. 2 and 3). The test compound is simultaneously compared against multiple classes of agents; thus, a separate probability is generated for each behavioral effect measured (e.g., anxiolytic activity, analgesic activity, etc.). In table 2 below, these probabilities are reported for each behavioral effect measured as described in Table 1 below.

TABLE 1

| Code | Class |
|---|---|
| AD | Antidepressant |
| AX | Anxiolytic |
| SH | Sedative-Hypnotic |
| AP | Antipsychotic |
| MS | Mood Stabilizer |
| PS | Psychostimulant |
| CE | Cognitive Enhancer |
| AN | Analgesic |
| AG | Anxiogenic |
| HL | Hallucinogen-Psychedelic |
| UK | Unknown |
| MED | Minimum Effective Dose (≥45% total activity) |
| ++++ | ≥74% |
| +++ | ≥49% |
| ++ | ≥24% |
| + | ≥4% |
| ND | <4% |

Provided compounds were dissolved in a mixture of Pharmasolve™ (N-methyl-2-pyrrolidone), polyethylene glycol and propylene glycol, and were injected i.p. 15 min. before the behavioral test. For each compound, injections were administered at 3 different doses. For each behavioral effect measured, results for the 3.0 mg/kg dose are presented. The potency of many of the compounds in the table was also determined in the SmartCube™ system. Test compounds were routinely examined at dose levels of 0.3, 1, 3 and 10 mg per kg (mpk), although the dose range was increased or decreased, if necessary, to obtain a full dose response curve. A compound's minimal effective dose (MED) is a measure of the compound's potency. The MED was defined as the dose (in mpk) having 45% or more total activity in SmartCube. The potencies of the compounds are shown in Table 2 below. If only one dose was tested, that dose is shown in the MED column of Table 2 even though the dose is not known to be the MED.

TABLE 2

| Compound | AD | AX | SH | AP | MS | PS | CE | AN | AG | HL | UK | MED |
|---|---|---|---|---|---|---|---|---|---|---|---|---|
| 9 | ++ | + | | ++ | ND | + | + | ND | ND | + | ND | 0.1 |
| 10 | ++ | ND | ND | ++ | ND | ND | ND | ND | ND | ND | ND | 0.3 |
| 11 | ND | ND | ND | ++++ | ND | ND | ND | ND | ND | ND | ND | 0.3 |
| 12 | ++ | ++ | ND | + | ND | ND | ND | ND | ND | ND | ND | 1.0 |
| 13 | ND | + | ND | +++ | ND | ND | + | ND | ND | ND | ND | 0.3 |
| 14 | ++ | + | ND | + | ND | ND | ND | ND | ND | ND | ND | 0.3 |
| 15 | ND | +++ | ND | + | ND | ND | ++ | ND | ND | ND | ND | 0.3 |
| 16 | ND | ++++ | ND | ND | ND | ND | ND | ND | ND | ND | ND | 1.0 |
| 17 | ND | ++++ | ND | +++ | ND | ND | ND | ND | ND | ND | ND | 1.0 |
| 18 | ++++ | ND | ND | + | ND | ND | ND | ND | ND | ND | ND | 0.3 |
| 19 | ++ | ++ | ND | + | ND | ND | ND | ND | ND | ND | + | 3.0 |
| 20 | ++++ | + | ND | + | ND | ND | + | ND | ND | ND | ND | 1.0 |
| 21 | ND | ND | ND | ND | ND | ND | ND | ND | ND | ND | +++ | 3.0 |
| 22 | ND | ND | ND | ND | ND | ND | ND | ND | ND | ND | + | >10 |
| 23 | ND | ND | ND | ND | ND | ND | ND | ND | ND | ND | ND | >10 |
| 24 | ND | ND | +++ | ND | ND | ND | ND | ND | ND | ND | ND | 3.0 |
| 25 | ND | ND | ND | ++++ | ND | ND | ND | ND | ND | ND | ND | 1.0 |
| 26 | ND | ND | + | ND | ND | ND | + | ND | ND | ND | ++ | 3.0 |
| 28 | +++ | ++ | ND | ND | ND | ND | ND | ND | ND | ND | ND | 3.0 |
| 29 | ND | ++ | ND | +++ | ND | ND | ND | ND | ND | ND | + | 1.0 |
| 30 | ND | ND | + | ++++ | ND | ND | ND | ND | ND | ND | ND | 0.3 |
| 31 | ND | ++ | ND | +++ | ND | ND | ND | ND | ND | ND | ND | 3.0 |
| 32 | +++ | ++ | ND | + | ND | ND | ND | ND | ND | ND | ND | 1.0 |
| 33 | +++ | ND | ND | ++ | ND | ND | ND | ND | ND | ND | ND | 0.3 |
| 34 | ND | ND | ND | ND | ND | ND | ND | ND | ND | ND | ++ | 10 |
| 35 | ND | ND | ND | ND | ND | ND | ND | ND | ND | ND | ++ | 10 |
| 36 | ND | ND | ND | +++ | ND | ND | ND | ND | ND | ND | ++ | 3.0 |
| 37 | ND | ND | ND | ND | ND | ND | ND | ND | ND | ND | +++ | 3.0 |
| 38 | ND | ND | ND | ND | ND | ND | ND | ND | ND | ND | ++ | 3.0 |
| 39 | +++ | ND | ND | ++ | ND | ND | ND | ND | ND | ND | ND | 3.0 |
| 40 | ND | +++ | ND | ++ | ND | ND | ND | ND | ND | ND | ND | 1.0 |
| 41 | +++ | ND | ND | ++ | ND | ND | ND | ND | ND | ND | ND | 1.0 |
| 42 | ++++ | ND | ND | ND | ND | ND | ND | ND | ND | ND | ND | 3.0 |
| 43 | ++++ | ND | ND | ND | ND | ND | ND | ND | ND | + | ND | 3.0 |
| 44 | ++ | ND | ND | +++ | ND | ND | ND | ND | ND | ND | ND | 1.0 |
| 45 | ++++ | ND | ND | + | ND | ND | ND | ND | ND | ND | ND | 0.1 |
| 46 | ND | + | ND | ND | ND | ND | ND | ND | ND | ND | ++ | 10 |
| 47 | ++++ | ND | ND | ND | ND | ND | ND | ND | ND | ND | ND | 0.3 |
| 49 | ++ | ++ | ND | ND | + | ND | + | ND | ND | ND | ND | 1.0 |
| 50 | +++ | ND | ND | ND | ND | ND | ND | ND | ND | ND | ND | 3.0 |
| 51 | ND | ND | ND | ND | ND | ND | ND | ND | ND | ND | + | >10 |
| 52 | ND | ++ | ND | +++ | ND | ND | ND | ND | ND | ND | + | 3.0 |
| 55 | ++ | + | ND | ++ | ND | ND | ND | ND | ND | ND | ND | 1.0 |
| 56 | ND | ++ | ND | ++ | ND | ND | ND | + | ND | ND | ND | 1.0 |
| 57 | ++ | ND | ND | ++ | ND | ND | ND | ND | ND | ND | + | 1.0 |
| 58 | + | ND | ND | +++ | + | ND | ND | ND | ND | ND | ND | 1.0 |
| 59 | ND | ++ | ND | +++ | ND | ND | ND | ND | ND | ND | ND | 3.0 |
| 60 | ND | +++ | ND | + | ND | ND | ND | ND | ND | ND | ND | 3.0 |
| 61 | + | ND | ND | +++ | ND | ND | ND | ND | ND | ND | ND | 3.0 |
| 62 | ++++ | ND | ND | + | ND | ND | ND | ND | ND | ND | ND | 1 |
| 63 | + | +++ | ND | ND | + | ND | ND | ND | ND | ND | ND | 1.0 |
| 64 | + | ND | ND | ND | ND | ND | ND | ND | ND | ND | ++ | 10 |
| 65 | +++ | ND | ND | ND | ND | + | ND | ND | ND | + | ND | 3 |
| 66 | ND | ND | ND | ++++ | ND | ND | ND | ND | ND | ND | ND | 1 |
| 67 | +++ | ND | ND | ND | ND | ND | ND | ND | ND | ND | ND | 3 |
| 68 | ++ | ND | ND | ND | ND | ND | ND | ND | ND | + | ND | 3 |
| 69 | ND | ND | ND | ND | ND | ND | ND | ND | ND | ND | +++ | 3 |
| 70 | ND | + | ND | +++ | ND | ND | ND | ND | ND | ND | ND | 1 |
| 73 | ND | ++++ | ND | ND | ND | ND | ND | ND | ND | ND | ND | 1 |
| 74 | + | ND | ND | ++++ | ND | ND | ND | ND | ND | ND | ND | 3 |
| 75 | ND | ND | ND | ++++ | ND | ND | ND | ND | ND | ND | ND | 1 |
| 76 | ND | ++ | ND | ++ | ND | ND | ND | ND | ND | ND | ND | 3 |
| 77 | ++ | + | ND | ++ | ND | ND | ND | ND | ND | ND | ND | 0.3 |
| 78 | ND | ++ | ND | ++ | ND | ND | ND | ND | ND | ND | + | 1 |
| 79 | ++ | ++ | ND | + | ND | ND | ND | ND | ND | ND | ND | 1 |
| 80 | ++ | + | ND | + | ND | ND | ND | ND | ND | ND | ND | 3 |
| 81 | ND | ND | ND | ++++ | ND | ND | ND | ND | ND | ND | ND | 1 |
| 82 | ++ | ND | ND | + | ND | ND | ND | ND | ND | ND | ++ | 3 |
| 83 | ND | + | ND | +++ | ND | ND | + | ND | ND | ND | ND | 1 |
| 84 | + | ND | +++ | ND | ND | ND | ND | ND | ND | ND | ND | 0.1 |
| 87 | ND | ND | ND | +++ | ND | ND | ND | ND | ND | ND | ND | 3 |
| 88 | ND | ND | ND | ++++ | ND | ND | ND | ND | ND | ND | ND | 3 |
| 89 | ND | + | ND | ND | ++ | ND | ND | ND | ND | ND | ND | 3 |
| 90 | ND | ND | ND | +++ | ND | ND | ND | ND | ND | ND | ND | 3 |
| 91 | ND | ND | ND | ND | ND | ND | ND | ND | ND | ND | ++ | >3 |
| 92 | ++++ | ND | ND | ND | ND | ND | ND | ND | ND | ND | ND | 3 |
| 93 | ND | ND | ND | ++++ | ND | ND | ND | ND | ND | ND | ND | 0.1 |
| 94 | ND | ND | ND | ++++ | ND | ND | ND | ND | ND | ND | ND | 3 |
| 95 | +++ | ND | ND | ++ | ND | ND | ND | ND | ND | ND | ND | 1 |
| 96 | ND | ND | ND | +++ | ND | ND | ND | ND | ND | ND | ND | 3 |
| 97 | ++++ | ND | ND | ND | ND | ND | ND | ND | ND | ND | ND | 1 |
| 98 | ND | + | ND | ++ | ND | ND | ND | ND | ND | ND | ++ | 3 |
| 99 | ND | ND | ND | ND | ND | ND | ND | ND | ND | ND | +++ | 3 |
| 100 | ND | ND | ND | ++++ | ND | ND | ND | ND | ND | ND | ND | 1 |
| 101 | ++++ | ND | ND | ND | ND | ND | ND | ND | ND | ND | ND | 3 |
| 102 | ++++ | ND | ND | ND | ND | + | ND | ND | ND | ND | ND | 1 |
| 103 | ND | ND | ND | ND | ND | ND | ND | ND | ND | ND | +++ | 1 |
| 104 | ND | ND | ND | ++ | ND | ND | ND | ND | ND | ND | ++ | 3 |
| 105 | ND | ND | ND | ND | ND | ND | ND | ND | ND | ND | ND | >3 |
| 106 | + | ND | ND | ND | ND | ND | ND | ND | ND | ND | ++ | 3 |
| 107 | ND | ND | ND | ND | ND | ND | ND | ND | ND | ND | ND | 10 |
| 108 | ND | ND | ND | ++++ | ND | ND | ND | ND | ND | ND | ND | <3 |
| 109 | ++++ | ND | ND | ND | ND | ND | ND | ND | ND | ND | ND | <3 |
| 111 | ++ | ++ | ND | ND | ND | + | ND | ND | ND | ND | ND | 3 |
| 112 | + | + | ND | ND | ND | ND | ND | ND | ND | ND | ND | >3 |
| 113 | + | ND | ND | ++++ | ND | ND | ND | ND | ND | ND | ND | 0.3 |
| 114 | +++ | ND | ND | + | ND | ND | ND | ND | ND | ND | ND | 3 |
| 115 | ND | ND | ND | ++++ | ND | ND | ND | ND | ND | ND | ND | 3 |
| 116 | ND | ND | ND | ND | ND | ND | ND | ND | ND | ND | ND | <3 |
| 117 | ND | ND | ND | ND | ND | ND | ND | ND | ND | ND | ND | >3 |
| 118 | ND | ND | ND | ND | ND | ND | ND | ND | ND | ND | + | >3 |
| 119 | ND | ++++ | ND | ND | ND | ND | ND | ND | ND | ND | ND | 3 |
| 120 | ND | ND | ND | ++++ | ND | ND | ND | ND | ND | ND | ND | 3 |
| 121 | ND | + | ND | + | ND | ND | ND | ND | ND | ND | +++ | 3 |
| 122 | +++ | + | ND | ND | ND | ND | ND | ND | ND | ND | ND | 1 |
| 123 | + | ND | ND | ND | ND | ++ | ND | ND | ND | ++ | ND | 1 |
| 124 | ND | ND | ND | ND | ND | ND | ND | ND | ND | ND | +++ | 3 |
| 125 | +++ | ND | ND | ND | ND | ND | ND | ND | ND | ND | ND | 0.3 |
| 126 | ND | ND | ND | ND | ND | ND | ND | ND | ND | ND | ND | >30 |
| 127 | ND | ND | ND | ND | ND | ND | ND | ND | ND | ND | ND | 30 |
| 128 | ND | ND | ND | ND | ND | ND | ND | ND | ND | ND | + | 10 |
| 129 | ND | ND | ND | ND | ND | ND | ND | ND | ND | ND | + | 10 |
| 130 | ND | ND | ND | ++++ | ND | ND | ND | ND | ND | ND | ND | <3 |
| 131 | + | ND | ND | ++ | + | ND | ND | ND | ND | ND | ND | 3 |
| 132 | ND | ND | ND | + | ND | + | ND | ND | ND | ND | ++ | 3 |
| 133 | +++ | ND | ND | + | ND | ND | ND | ND | ND | ND | ND | <1 |
| 134 | ND | ND | ND | ND | ND | ND | ND | ND | ND | ND | +++ | 3 |
| 135 | ND | ND | ++ | + | ND | ND | ND | ND | ND | ND | ND | 3 |
| 136 | ND | ND | ND | ND | ND | ND | ND | ND | ND | ND | + | >3 |
| 137 | ND | ND | ND | ++++ | ND | ND | ND | ND | ND | ND | ND | 1 |
| 138 | ND | ++ | ND | +++ | ND | ND | ND | ND | ND | ND | ND | 1 |
| 139 | ++ | ND | ND | ND | ND | ND | ND | ND | ND | ND | ++ | 0.3 |
| 140 | ND | ND | ND | + | ND | ND | ND | ND | ND | ND | +++ | 3 |
| 141 | + | +++ | ND | ND | ND | ND | ND | ND | ND | ND | ND | 3 |
| 142 | + | ND | ND | + | ND | + | ND | ++ | ND | ND | + | 1 |
| 143 | + | ND | + | ND | ND | ++ | ND | ND | ND | + | ND | 1 |
| 144 | ND | ND | ND | ND | ND | ND | ND | ND | ND | ND | ++++ | 1 |
| 145 | ++++ | ND | ND | ND | ND | ND | ND | ND | ND | ND | ND | 3 |
| 146 | ND | ND | ND | ND | ND | ND | ND | ND | ND | ND | +++ | 3 |
| 147 | ND | ND | ND | ND | ND | ND | ND | + | ND | + | +++ | 3 |
| 148 | +++ | ++ | ND | + | ND | ND | ND | ND | ND | ND | ND | 3 |
| 149 | ND | ND | ND | ND | ND | ++++ | ND | ND | ND | + | ND | 3 |
| 150 | ND | ND | ND | ND | ND | ++ | ND | ND | ND | +++ | ND | 3 |
| 151 | ND | ND | ND | ND | ND | ND | ND | ND | ND | ND | +++ | 3 |
| 152 | ND | ND | ND | ND | ND | ND | ND | ND | ND | ND | ++ | 3 |
| 153 | ++++ | ND | ND | ND | ND | ND | ND | ND | ND | ND | ND | 3 |
| 154 | ND | ND | ND | ND | ND | ND | ND | ND | ND | ND | ND | >1 |
| 155 | + | ND | ++ | ND | + | ND | + | ND | ND | ND | ND | 0.3 |
| 156 | ND | ++ | ND | ND | ND | ND | ND | ND | ND | ND | ++ | 3 |
| 157 | ND | +++ | ND | ND | ND | ND | ND | ND | ND | ND | ND | 3 |
| 158 | ND | ++++ | ND | ND | ND | ND | ND | ND | ND | ND | ND | 1 |
| 159 | ND | ND | ND | ND | ND | ND | ND | ND | ND | ND | + | >3 |
| 160 | ND | ND | ND | ND | ND | ND | ND | ND | ND | ND | + | >3 |
| 161 | ND | ND | ND | ND | ND | ND | ND | ND | ND | ND | ND | >3 |
| 162 | ND | +++ | ND | ND | ND | ND | ND | ND | ND | ND | + | 3 |
| 163 | ND | ND | ND | ND | ND | ND | ND | ND | ND | ND | + | >3 |
| 164 | +++ | ND | ND | ND | ND | ND | ND | ND | ND | ND | ND | 3 |
| 165 | ND | ND | ND | ND | ND | ND | ND | ND | ND | ++++ | ND | 3 |
| 166 | + | +++ | ND | ++ | ND | ND | ND | ND | ND | ND | ND | 1 |
| 167 | ++ | +++ | ND | ND | ND | + | ND | ND | ND | ND | ND | 1 |
| 168 | ND | ND | ND | +++ | ND | ND | ND | ND | ND | ND | ND | 3 |
| 169 | ND | ND | ND | ND | ND | ND | ND | ND | ND | ND | ++ | 3 |
| 170 | ND | ++ | ND | +++ | ND | ND | ND | ND | ND | ND | ND | 1 |
| 171 | ND | ND | ND | ++++ | ND | ND | ND | ND | ND | ND | ND | 3 |
| 172 | ND | ++++ | ND | ND | ND | ND | ND | ND | ND | ND | ND | 1 |
| 173 | ND | ND | ND | ND | ND | ND | ND | ND | ND | ND | ++ | >3 |
| 174 | ND | ND | + | ND | ND | ND | ND | ND | ND | ND | ND | 1 |
| 175 | ND | +++ | + | ND | ND | ND | ND | ND | ND | ND | ND | 3 |
| 176 | ND | ND | ND | ND | ND | ND | ND | ND | ND | ND | ND | >3 |
| 177 | ND | ND | ND | ND | ND | ND | ND | ND | ND | ND | ++++ | 3 |
| 178 | + | ND | ND | ND | ND | ++++ | ND | ND | ND | ND | ND | 1 |
| 179 | +++ | ND | ND | ND | ND | + | ND | + | ND | ND | ND | 3 |
| 180 | ND | ND | ND | +++ | ND | ND | ND | ND | ND | ND | ND | 3 |
| 181 | ND | ND | ND | + | ND | ND | ND | + | ND | ND | ND | >3 |
| 182 | ND | ND | + | ND | ND | + | ND | ND | ND | ND | ++ | 3 |
| 183 | ND | ND | ND | ND | ND | ND | ND | ND | ND | ND | ++ | 3 |
| 184 | ND | ND | ND | ND | ND | ND | ND | ND | ND | ND | ++ | >3 |
| 185 | ND | ND | ND | ND | ND | ND | ND | ND | ND | ND | ++ | |
| 186 | ND | ND | ND | ND | ND | ND | ND | ND | ND | ND | + | >3 |
| 187 | ND | ND | ND | ++ | ND | ND | ND | ND | ND | ND | ++ | 3 |
| 188 | ND | + | ND | ND | ND | ND | ND | ND | ND | ND | ++ | >3 |
| 189 | ND | ND | ND | ND | ND | ND | ND | ND | ND | ND | ++ | >3 |
| 190 | ND | ND | ND | ND | ND | ND | ND | ND | ND | ND | + | >3 |
| 191 | ++ | ND | ND | + | ND | + | + | + | + | ND | ND | 1 |
| 192 | ND | ND | ND | ND | ND | ND | ND | ND | ND | ND | +++ | 3 |
| 193 | ND | ND | ND | ND | ND | ND | ND | + | ND | ND | ND | >3 |
| 194 | ND | ND | ND | ++++ | ND | ND | ND | ND | ND | ND | ND | 3 |
| 195 | ND | ND | ND | ND | ND | ND | ND | ND | ND | ND | + | >3 |
| 196 | ND | ND | ND | ND | ND | ND | ND | ND | ND | ND | + | 3 |
| 197 | ND | ND | ND | ND | ND | ND | ND | ND | ND | ND | +++ | 3 |
| 198 | ND | ND | ND | ++++ | ND | ND | ND | ND | ND | ND | ND | <3 |
| 199 | ND | ND | ND | ND | ND | ND | ND | ND | ND | + | ND | <3 |
| 200 | ND | ND | ND | ND | ND | ++ | ND | ++ | ND | + | ND | 3 |
| 201 | ND | + | ND | ND | ND | ND | ND | ND | ND | ND | ++ | 3 |
| 202 | + | ++ | ND | ++ | ND | ND | ND | ND | ND | ND | ND | 3 |
| 203 | ND | ND | ND | ND | ND | ND | ND | ND | ND | ND | +++ | 3 |
| 204 | ND | ND | ND | ++++ | ND | ND | ND | ND | ND | ND | ND | 3 |
| 205 | ND | ND | ND | ND | ND | ND | ND | ND | ND | ND | ++ | >3 |
| 206 | ND | ND | ND | ND | ND | ND | ND | ND | ND | ND | ++ | 3 |
| 207 | ND | ND | ND | ND | ND | ND | ND | ND | ND | ND | + | >3 |
| 208 | ND | ND | ND | ND | ND | ND | ND | ND | ND | ND | + | >3 |
| 209 | ++ | +++ | ND | + | ND | ND | + | ND | ND | ND | ND | <3 |
| 210 | ND | ND | ND | + | ND | ND | ND | ND | ND | ND | +++ | 3 |
| 211 | ND | ND | ND | ++++ | ND | ND | ND | ND | ND | ND | ND | <3 |
| 212 | +++ | + | ND | + | ND | ND | + | ND | ND | ND | ND | 1 |
| 213 | + | +++ | + | + | ND | ND | ND | ND | ND | ND | ND | 1 |
| 214 | ND | ND | ND | ND | ND | ND | ND | ND | ND | ND | + | >3 |
| 215 | ND | +++ | ND | + | ND | ND | ND | ND | ND | ND | ND | 3 |
| 216 | ND | ++ | ND | ND | ND | ND | ND | ND | ND | ND | + | >3 |
| 217 | ND | ++ | ND | + | ND | ++ | ND | ND | ND | + | + | <3 |
| 219 | ND | ND | ND | ND | ND | ND | ND | ND | ND | ND | ++ | >3 |
| 220 | ND | ND | ND | ND | ND | ND | ND | ND | ND | ND | ++ | >3 |
| 221 | ND | ++ | ND | ND | ND | ND | ND | + | ND | + | ++ | 3 |
| 222 | ND | ND | ND | ++++ | ND | ND | ND | ND | ND | ND | ND | <3 |
| 223 | ND | ND | ND | ND | ND | ND | ND | ND | ND | ND | ++ | >3 |
| 224 | ND | ND | ND | ++++ | ND | ND | ND | ND | ND | ND | ND | 3 |
| 225 | ND | ND | ND | ++++ | ND | ND | ND | ND | ND | ND | ND | <3 |
| 228 | ND | ND | ND | ND | ND | ND | ND | ND | ND | ND | ND | 1 |
| 229 | N/A | ++++ | ND | ND | ND | ND | ND | ND | ND | + | ND | 3 |
| 230 | + | ND | ND | ND | ND | ND | ND | ND | ND | ND | ND | 3 |
| 231 | ND | ND | ND | ND | ND | ND | ND | ND | ND | ND | +++ | 3 |
| 232 | ND | ND | ND | ND | ND | ND | ND | ND | ND | ND | + | >3 |
| 233 | ND | ND | ND | ND | ND | ND | ND | ND | ND | ND | ND | >3 |
| 234 | ND | + | ND | ++++ | ND | ND | ND | ND | ND | ND | ND | 1 |
| 235 | ++++ | ND | ND | ND | ND | ND | ND | ND | ND | ND | ND | 3 |
| 236 | ND | ND | ND | ND | ND | ND | ND | ND | ND | ND | ND | >3 |
| 237 | ND | ND | ND | ND | ND | ND | ND | ND | ND | ND | + | >3 |
| 238 | ND | ND | ND | ND | ND | ND | ND | ND | ND | ND | ++ | >3 |
| 239 | ND | ND | ND | ND | ND | ND | ND | ND | ND | ND | ++ | >3 |
| 240 | + | ND | ND | ND | ND | ND | ND | ND | ND | ND | +++ | 3 |
| 241 | + | ND | ND | ND | ND | ND | ND | ND | ND | ND | ++++ | 3 |
| 242 | +++ | ND | ND | ND | ND | ND | ND | ND | ND | ND | ND | 3 |
| 243 | ++ | ND | ND | ND | ND | ND | ND | ND | ND | ND | ND | 3 |
| 244 | ++++ | ND | ND | ND | ND | ND | ND | ND | ND | ND | ND | <3 |
| 245 | ++ | ND | ND | ND | ND | ND | ND | ND | ND | ND | + | 3 |
| 246 | + | ND | ND | ++ | ND | ND | ND | ND | ND | ND | ++ | 3 |
| 247 | ND | ND | ND | ++++ | ND | ND | ND | ND | ND | ND | ND | 3 |
| 248 | +++ | ND | ND | ND | ND | ND | ND | ND | DN | ND | ND | 1 |
| 249 | ND | ND | ND | ND | ND | ND | ND | ND | ND | ND | ++ | 3 |
| 250 | ND | ++++ | ND | + | ND | ND | ND | ND | ND | ND | ND | 3 |
| 251 | ND | ND | ND | ND | ND | DN | DN | ND | ND | ND | ++ | >3 |
| 252 | ND | ND | ND | + | ND | ND | ND | ND | ND | ND | +++ | 3 |
| 254 | ND | ND | ND | ND | ND | ND | ND | ND | ND | ND | + | >3 |
| 255 | ND | ND | ND | ND | ND | ND | ND | ND | ND | ND | ND | >3 |
| 256 | ND | ND | ND | ND | ND | ND | ND | ND | ND | ND | + | >3 |
| 257 | ND | ND | ND | ND | ND | ND | ND | ND | ND | ND | ++ | >3 |
| 258 | ND | ND | ND | ND | ND | ND | ND | ND | ND | ND | + | >3 |
| 259 | ND | ND | ND | ND | ND | ND | ND | ND | ND | ND | ++ | >3 |
| 260 | + | ND | ND | ND | ND | ND | ND | ND | ND | ND | +++ | 3 |
| 261 | +++ | ND | ND | ND | ND | ND | ND | ND | ND | ND | + | 3 |
| 262 | ++ | ND | ND | ND | ND | ND | ND | ND | ND | ND | ND | >3 |
| 266 | ND | ND | ND | ND | ND | ND | ND | ND | ND | DN | + | >3 |
| 267 | ND | ND | ND | ND | ND | ND | ND | ND | ND | ND | + | >3 |
| 268 | ND | ND | ND | ND | ND | ND | ND | ND | ND | ND | ND | >3 |
| 275 | ND | ND | ND | ND | ND | ++++ | ND | ND | ND | ND | ND | 3 |
| 276 | ND | ++++ | ND | ND | ND | ND | ND | ND | ND | ND | ND | 3 |
| 277 | ND | ND | ND | ND | ND | ND | ND | ND | ND | ND | + | >3 |
| 278 | ND | ND | ND | ND | ND | ND | ND | ND | ND | ND | + | >3 |
| 279 | ND | ND | ND | ND | ND | ND | ND | ND | ND | ND | ++ | 3 |
| 280 | ND | ND | ND | ND | ND | ND | ND | ND | ND | ND | +++ | 3 |
| 281 | ++++ | + | ND | + | ND | ND | ND | ND | ND | ND | ND | 3 |
| 282 | ++++ | ND | ND | ND | ND | ND | ND | ND | ND | ND | ND | 3 |
| 283 | ND | ND | ND | ND | ND | ND | ND | ND | ND | ND | + | 3 |
| 284 | ND | ND | ND | ND | ND | ND | ND | ND | ND | ND | ++ | 3 |
| 285 | ND | ND | ND | ND | ND | ND | ND | ND | ND | ND | +++ | 3 |
| 288 | ND | ND | ND | ND | ND | ND | ND | ND | ND | ND | ND | >3 |
| 289 | ND | ND | ND | ND | ND | ND | ND | ND | ND | ND | + | >3 |
| 290 | ND | ND | ND | ND | ND | ND | ND | ND | ND | ND | + | >3 |
| 291 | ND | ND | ND | ND | ND | ND | ND | ND | ND | ND | + | >3 |
| 292 | ND | ND | ND | ND | ND | ND | ND | ND | ND | ND | ++ | >3 |
| 293 | ND | ND | ND | ++++ | ND | ND | ND | ND | ND | ND | ND | <3 |
| 294 | ND | ND | ND | ND | ND | ND | ND | ND | ND | ND | + | >3 |
| 295 | ND | ND | ND | +++ | ND | ND | ND | ND | ND | ND | ND | 3 |
| 296 | ND | ND | ND | ND | ND | ND | ND | ND | ND | ND | + | >3 |
| 297 | ND | ND | ND | ND | ND | ND | ND | ND | ND | ND | ND | 3 |
| 298 | ND | +++ | ND | ND | ND | ND | ND | ND | ND | ND | ND | 3 |
| 308 | ND | ND | ND | ++++ | ND | ND | ND | ND | ND | ND | ND | <3 |
| 309 | ND | ND | ND | ND | ND | ND | ND | ND | ND | ND | ND | 1 |
| 310 | ND | ND | ND | ND | ND | ND | ND | ND | ND | ND | ++ | >3 |
| 311 | ++ | ND | ND | ND | ND | ND | ND | ND | ND | ND | +++ | 3 |
| 312 | ND | ND | ND | ND | ND | ND | ND | ND | ND | ND | +++ | 3 |
| 313 | ND | ND | ND | ND | ND | ND | ND | ND | ND | ND | + | >3 |
| 314 | ND | ND | ND | ND | ND | ND | ND | ND | ND | ND | + | >3 |
| 315 | ND | ++++ | ND | + | ND | ND | ND | ND | ND | ND | ND | 3 |
| 316 | ND | ND | ND | ++++ | ND | ND | ND | ND | ND | ND | ND | 3 |
| 317 | ND | +++ | ND | + | ND | ND | + | ND | ND | ND | ND | 3 |
| 318 | ND | + | ND | + | ND | ND | ND | ND | ND | ND | +++ | 3 |
| 319 | ND | ++++ | ND | ND | ND | ND | + | ND | ND | ND | ND | 3 |
| 320 | ND | ND | ND | ND | ND | ND | ND | ND | ND | ND | ++ | >3 |
| 321 | ND | ++++ | ND | + | ND | ND | ND | ND | ND | ND | ND | 3 |
| 322 | ND | ND | ND | +++ | ND | ND | ND | ND | ND | ND | ++ | 3 |
| 323 | ++ | + | ND | ++ | ND | ND | ND | ND | ND | ND | + | 3 |
| 324 | + | ND | ND | ND | ND | ND | ND | ND | ND | ND | ND | 3 |
| 325 | ND | ND | ND | ND | ND | ND | ND | ND | ND | ND | ++ | 3 |
| 326 | ND | ++ | ND | ND | ND | ND | ND | ND | ND | ND | ++ | 3 |
| 327 | ND | ++ | ND | ND | ND | ND | ND | ND | ND | ND | +++ | 3 |
| 328 | ++ | + | ND | ND | ND | ++ | ND | ND | ND | + | + | 3 |
| 329 | ++++ | ND | ND | ND | ND | ND | ND | ND | ND | ND | ND | <3 |
| 330 | ND | ND | ND | ND | ND | ND | ND | ND | ND | ND | + | >3 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

What is claimed is:

1. A compound of Formula I: 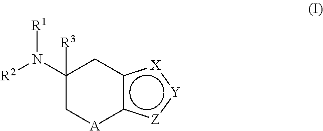
or a pharmaceutically acceptable salt thereof, wherein:
X is S, Y is CR⁵, and Z is CR⁶; or X is CR⁴, Y is S, and Z is CR⁶; or X is CR⁴, Y is CR⁵, and Z is S;
A is chosen from a single bond, —CH₂—, —O—, —CH₂—CH₂—, and —CH═CH—;
R¹ is chosen from H, methyl, and ethyl, and R² is chosen from H, C₁-C₃ alkyl optionally substituted by one C₃-C₅ saturated cycloalkyl, 3-5 membered saturated heterocycloalkyl, and C₃-C₅ saturated cycloalkyl optionally substituted by one or more halogen, or R¹ and R², taken together with the intervening nitrogen atom, form a 3-7 membered saturated heterocyclic ring;
R³ is H or C₁-C₃ alkyl;
R⁴, R⁵, and R⁶ are independently chosen from H, C₁-C₄ alkyl optionally substituted by one NR¹⁰R¹¹, C₁-C₄ haloalkyl, halogen, CN, phenyl, and C₃-C₅ saturated cycloalkyl; and
R¹⁰ and R¹¹ are independently chosen from H and C₁-C₄ alkyl;
provided that:
a) R² is H only if R¹ is H;
b) if A is —CH₂—, X is S, and R¹, R², R³ and R⁵ are H, then R⁶ is not H, methyl, CN, cyclopentyl, isobutyl, t-butyl, phenyl, CF₃, or CH₂CH₂N(CH₃)₂;
c) if A is —CH₂—, Y is S, and R¹, R², R³ and R⁶ are H, then R⁴ is not H or Br;
d) if A is —CH₂—, Z is S, and R¹, R², R³ and R⁴ are H, then R⁵ is not H, CN, Cl, or Br;
e) if A is —CH₂—, Z is S, and R¹, R³ and R⁴ are H, and R⁵ is H, Cl, or Br, then R² is not methyl; and
f) the compound of Formula I is not

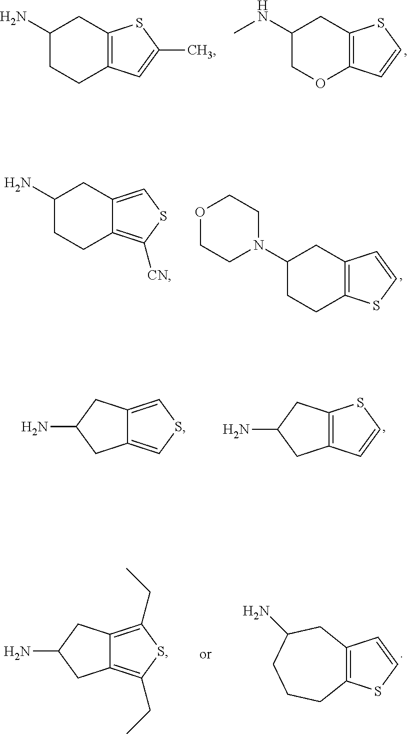

2. The compound of claim 1, or a pharmaceutically acceptable salt thereof, wherein
a) if A is —CH₂—, X is S, and R² is C₁-C₃ alkyl optionally substituted by one C₃-C₅ saturated cycloalkyl, then at least one of R¹, R³, R⁵ and R⁶ is not H; and
b) if A is —CH₂—, Z is S, and R² is C₁-C₃ alkyl optionally substituted by one C₃-C₅ saturated cycloalkyl, then at least one of R¹, R³, R⁴ and R⁵ is not H.

3. The compound of claim 1, or a pharmaceutically acceptable salt thereof, wherein A is —CH₂— or —O—.

4. The compound of claim 1, or a pharmaceutically acceptable salt thereof, wherein R¹ is chosen from H, methyl, and ethyl, and R² is chosen from H, C₃-C₅ saturated cycloalkyl, and C₁-C₂ alkyl optionally substituted by one C₃-C₅ saturated cycloalkyl, or R¹ and R², taken together with the intervening nitrogen atom, form a 3-7 membered saturated heterocyclic ring.

5. The compound of claim 1, or a pharmaceutically acceptable salt thereof, wherein R³ is H or methyl.

6. The compound of claim 1, or a pharmaceutically acceptable salt thereof, wherein R⁴, R⁵, and R⁶ are independently chosen from H, C₁-C₃ alkyl, C₁-C₃ haloalkyl, halogen, CN, and C₃-C₅ saturated cycloalkyl.

7. The compound of claim 1, or a pharmaceutically acceptable salt thereof, wherein Y is S.

8. The compound of claim 1, or a pharmaceutically acceptable salt thereof, wherein X is S.

9. The compound of claim 1, or a pharmaceutically acceptable salt thereof, wherein Z is S.

10. The compound of claim 8, or a pharmaceutically acceptable salt thereof, wherein R⁶ is not H.

11. The compound of claim 9, or a pharmaceutically acceptable salt thereof, wherein R⁴ is not H.

12. The compound of claim 1, or a pharmaceutically acceptable salt thereof, wherein R⁵ is H.

13. The compound of claim 1, chosen from: 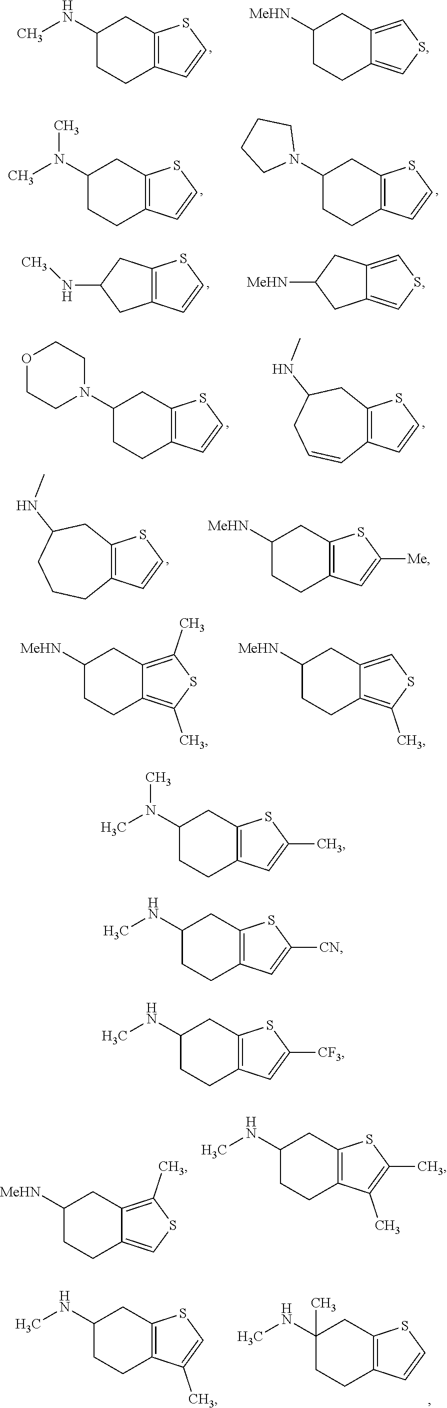 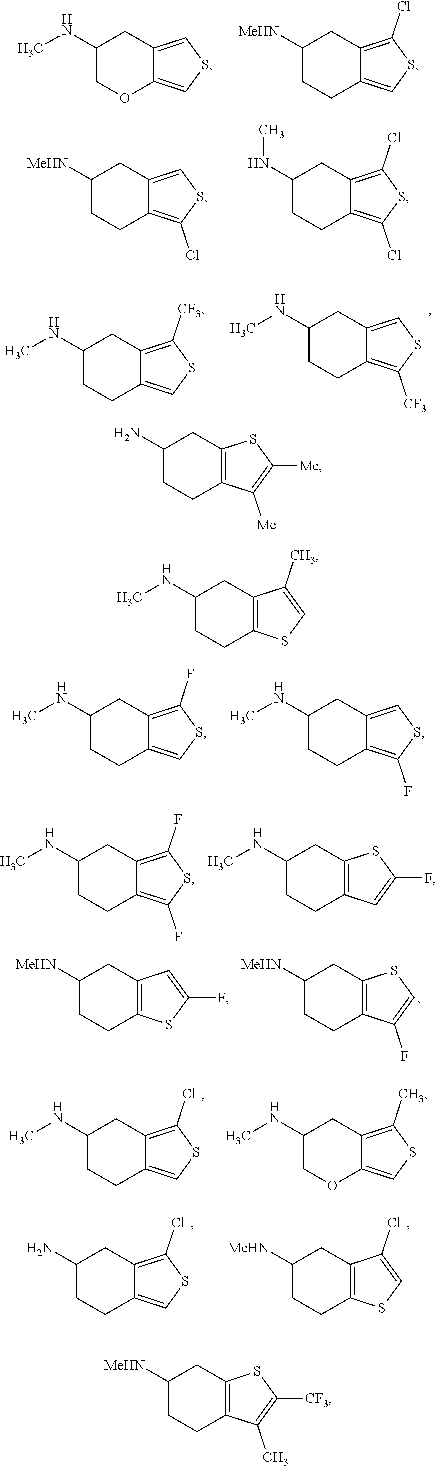 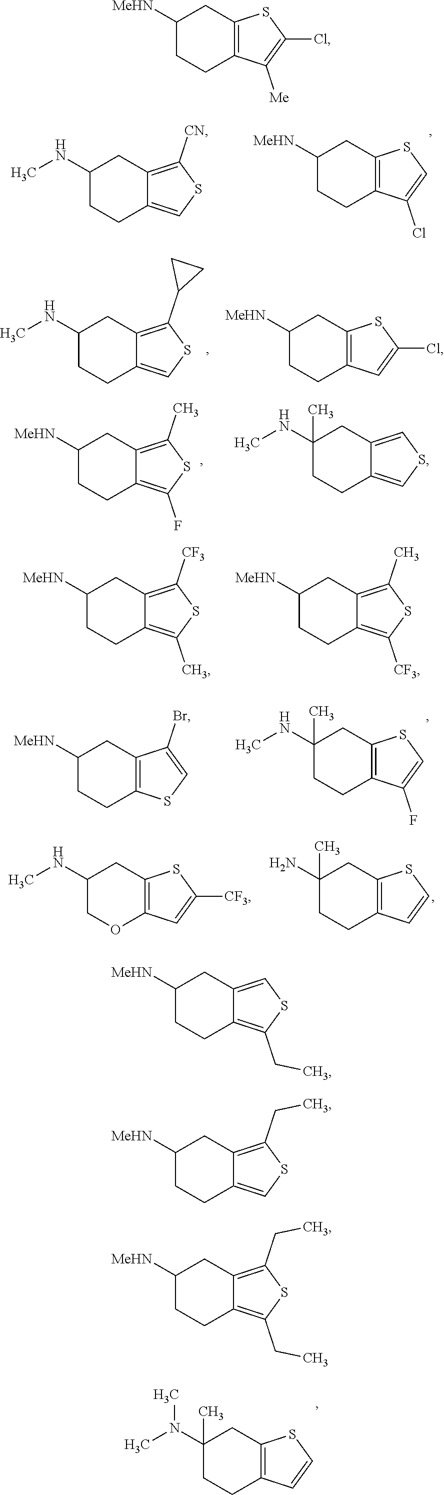 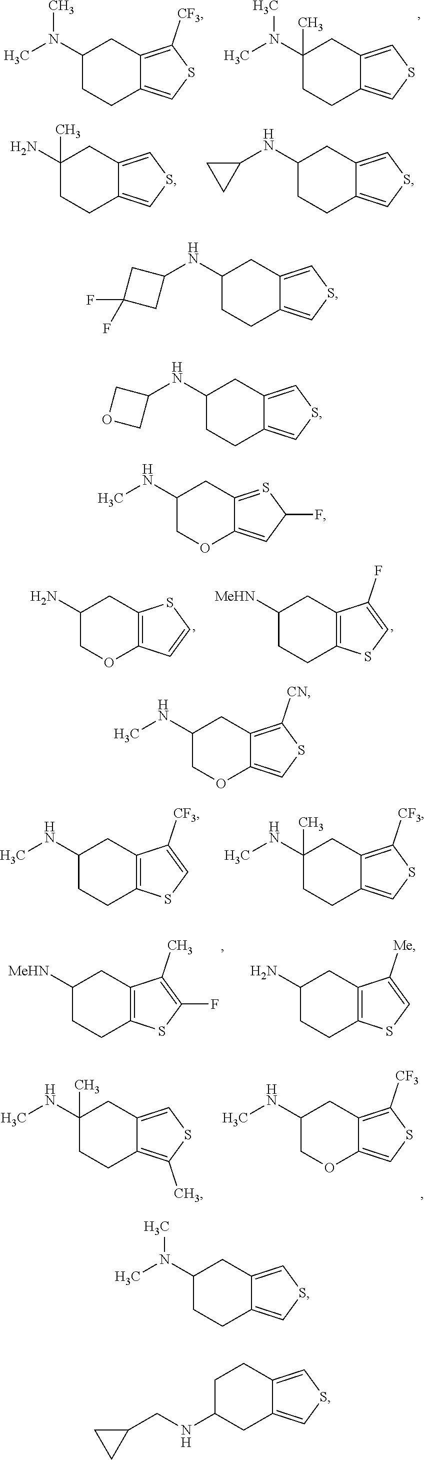 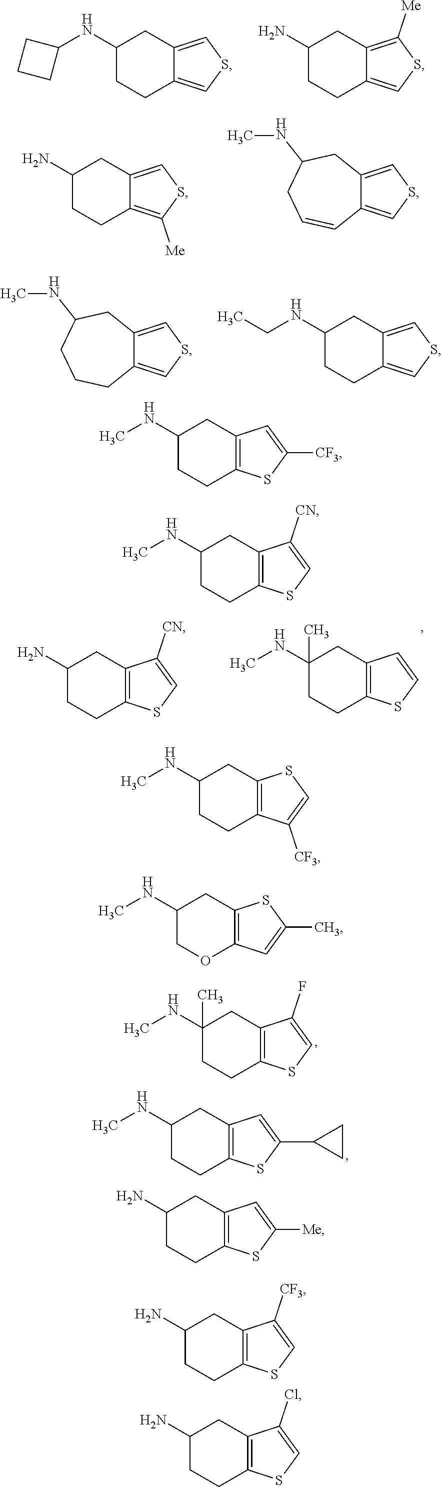 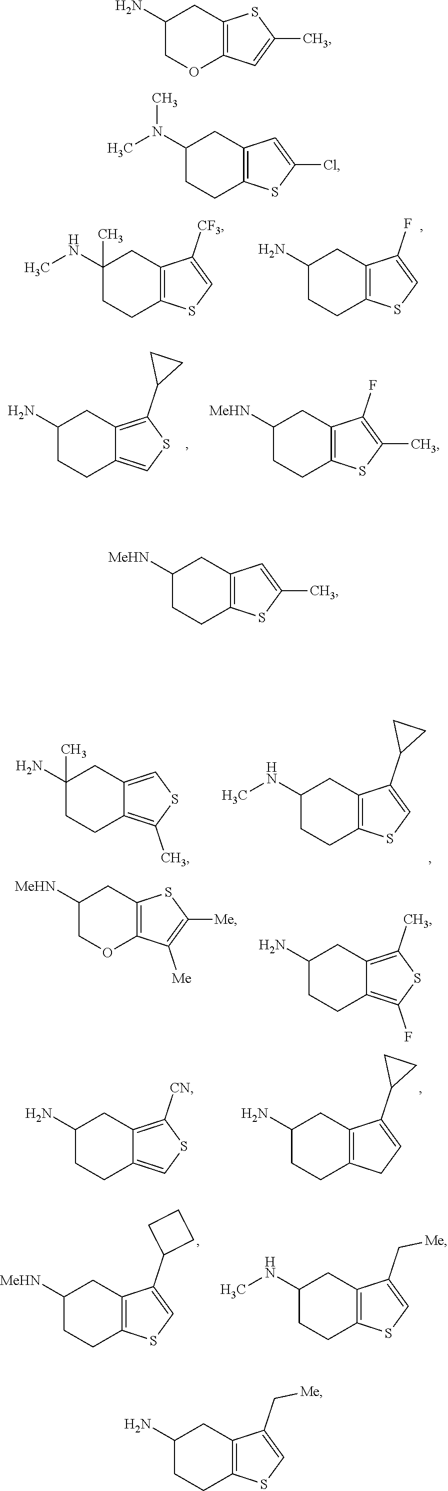 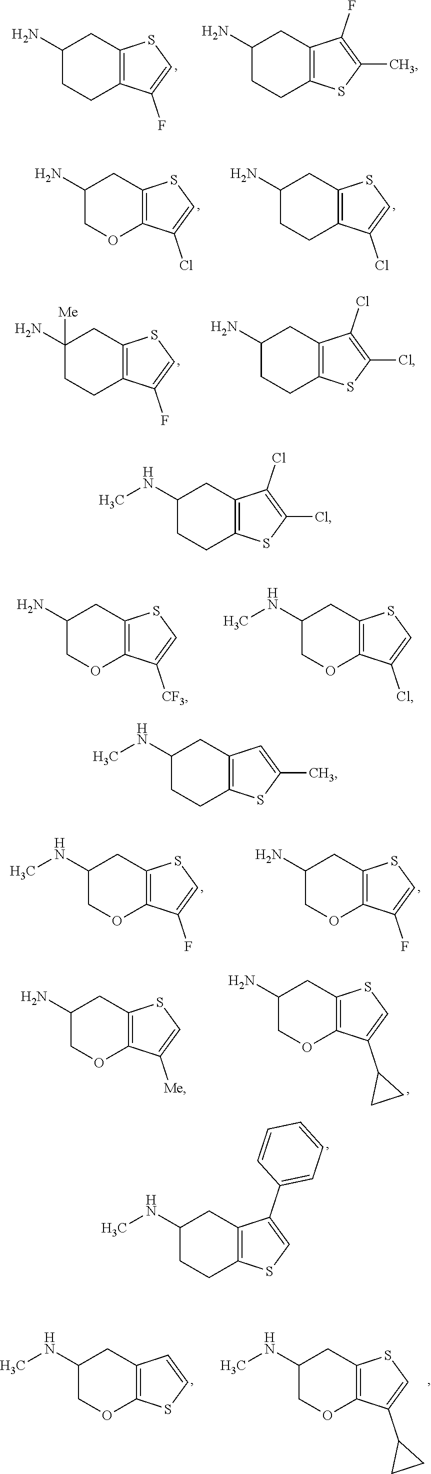 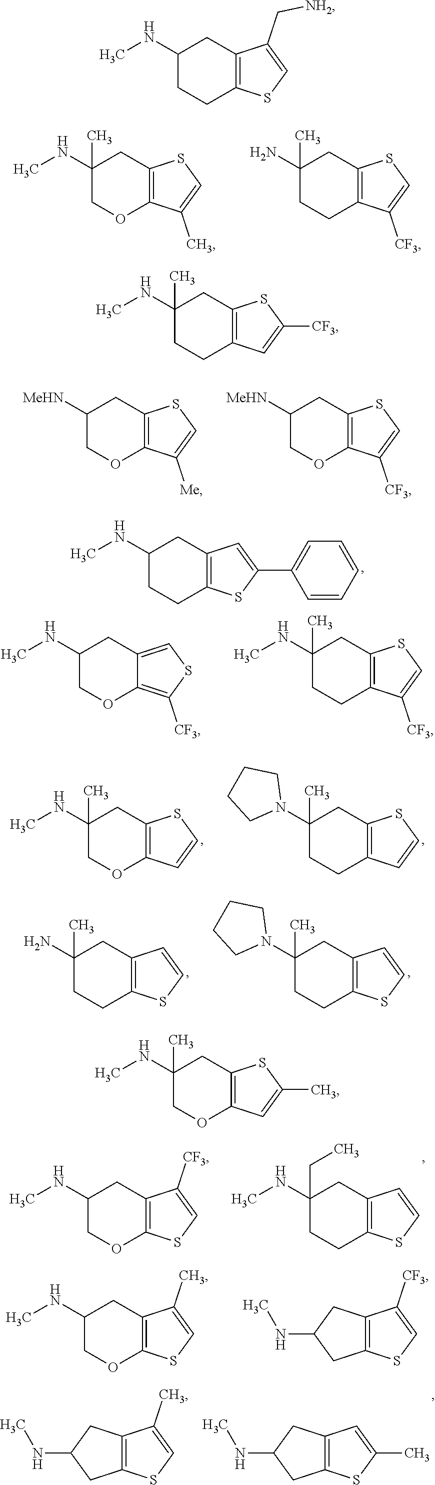 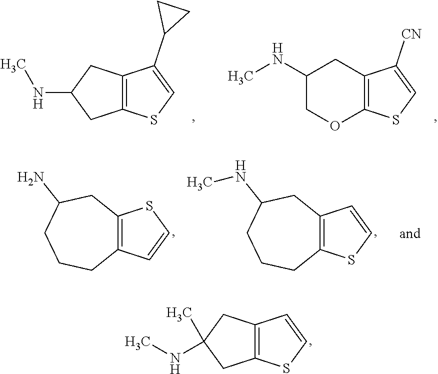
or a pharmaceutically acceptable salt thereof.

14. The compound of claim 1, or a pharmaceutically acceptable salt thereof, wherein the compound is at least 90% enantiomerically pure.

15. A compound chosen from N-methyl-4,5,6,7-tetrahydrobenzothiophen-5-amine, 3-methyl-4,5,6,7-tetrahydrobenzothiophen-6-amine, 2-chloro-N-methyl-4,5,6,7-tetrahydrobenzothiophen-5-amine, 4,5,6,7-tetrahydro-2-benzothiophen-5-amine, 4,5,6,7-tetrahydrobenzothiophen-5-amine, 2-chloro-4,5,6,7-tetrahydrobenzothiophen-5-amine, 3-(trifluoromethyl)-4,5,6,7-tetrahydrobenzothiophen-6-amine, 5,6-dihydro-4H-cyclopenta[b]thiophen-5-amine, and N-methyl-5,6,7,8-tetrahydro-4H-cyclohepta[b]thiophen-6-amine, or a pharmaceutically acceptable salt thereof, wherein the compound is at least 90% enantiomerically pure.

16. A pharmaceutical composition comprising a compound of claim 1, or pharmaceutically acceptable salt thereof, together with one or more pharmaceutically acceptable excipients.

17. A pharmaceutical composition comprising a compound chosen from N-methyl-4,5,6,7-tetrahydrobenzothiophen-5-amine, 3-methyl-4,5,6,7-tetrahydrobenzothiophen-6-amine, 2-chloro-N-methyl-4,5,6,7-tetrahydrobenzothiophen-5-amine, 4,5,6,7-tetrahydro-2-benzothiophen-5-amine, 4,5,6,7-tetrahydrobenzothiophen-5-amine, 2-chloro-4,5,6,7-tetrahydrobenzothiophen-5-amine, 3-(trifluoromethyl)-4,5,6,7-tetrahydrobenzothiophen-6-amine, 5,6-dihydro-4H-cyclopenta[b]thiophen-5-amine, and N-methyl-5,6,7,8-tetrahydro-4H-cyclohepta[b]thiophen-6-amine, or a pharmaceutically acceptable salt thereof, together with one or more pharmaceutically acceptable excipients.

18. A method of treating a central nervous system disease or disorder in a patient in need thereof, comprising administering to the patient a therapeutically effective amount of a compound of claim 1, or pharmaceutically acceptable salt thereof, wherein the central nervous system disease or disorder is depression, schizophrenia, or an anxiety disorder.

19. A method of treating a central nervous system disease or disorder in a patient in need thereof, comprising administering to the patient a therapeutically effective amount of a compound of claim 14, or pharmaceutically acceptable salt thereof, wherein the central nervous system disease or disorder is depression, schizophrenia, or an anxiety disorder.

20. A method of treating a central nervous system disease or disorder in a patient in need thereof, comprising administering to the patient a therapeutically effective amount of a compound of claim 15, or pharmaceutically acceptable salt thereof, wherein the central nervous system disease or disorder is depression, schizophrenia, or an anxiety disorder.
